(19) United States
(12) Patent Application Publication
(10) Pub. No.: US 2026/0014147 A1
(43) Pub. Date: Jan. 15, 2026

(54) STAT6 DEGRADERS

(71) Applicant: Gilead Sciences, Inc., Foster City, CA (US)

(72) Inventors: Patrick Stephen Johnson, Valby (DK); Geanna Min, Herlev (DK); Thomas Holmstrøm, København S (DK); Sebastian Clementson, Malmö (SE); Pablo Martín-Gago, Frederiksberg (DK); Kristoffer Månsson, Hjärup (SE); Morten Jørgensen, Herlev (DK); Kevin Neil Dack, Kongens Lyngby (DK); Marcel John de Groot, Værløse (DK)

(21) Appl. No.: 19/091,645

(22) Filed: Mar. 26, 2025

(30) Foreign Application Priority Data
Mar. 27, 2024 (EP) EP24167017.3

(51) Int. Cl. A61K 31/4985; A61K 31/4545; A61K 31/496; A61K 31/501; A61K 31/5025; A61K 31/506; A61K 31/513; C07D 471/04; C07D 519/00

(52) U.S. Cl. CPC A61K 31/4985; A61K 31/4545; A61K 31/496; A61K 31/501; A61K 31/5025; A61K 31/506; A61K 31/513; C07D 471/04; C07D 519/00

(57) ABSTRACT

The present disclosure relates to a compound according to formula (1):

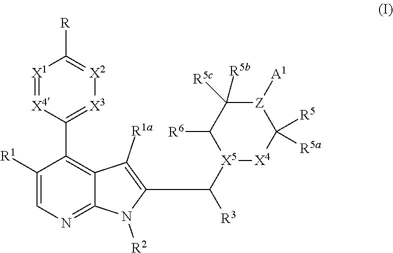

and pharmaceutically acceptable salts, hydrates, or solvates thereof. The disclosure further relates to said compounds for use in therapy, to pharmaceutical compositions comprising said compounds, to methods of treating diseases, e.g. dermal diseases, with said compounds, and to the use of said compounds in the manufacture of medicaments.

### CROSS-REFERENCE TO RELATED APPLICATIONS

This application claims priority to European Patent Application No. EP24167017.3, filed Mar. 27, 2024, which is incorporated herein in its entirety for all purposes.

### FIELD

Provided herein are novel azaindole compounds and pharmaceutically acceptable salts thereof that modulate STAT6 and pharmaceutical compositions comprising said compounds for use in therapy (e.g., for treating STAT6 associated diseases in a subject in need thereof).

### BACKGROUND

The disclosure relates generally to methods and compounds, and pharmaceutically acceptable salts thereof, for modulating a Signal transducer and activator of transcription 6 (STAT6) protein activity and treating STAT6associated diseases. The following description sets forth exemplary methods, parameters and the like. It should be recognized, however, that such description is not intended as a limitation on the scope of the present disclosure but is instead provided as a description of exemplary embodiments.

The present disclosure relates novel bifunctional compounds and pharmaceutically acceptable salts thereof, which may function to recruit STAT6proteins to E3ubiquitin ligase for degradation, compositions containing such compounds and methods and uses thereof. In particular, the present disclosure provides bifunctional compounds and pharmaceutically acceptable salts thereof, which find utility as modulators of targeted ubiquitination of STAT6proteins, which are then degraded and/or otherwise inhibited by the bifunctional compounds as described herein.

Ubiquitin-Proteasome Pathway (UPP) is a pathway that regulates key regulator proteins and degrades proteins, such as misfolded or abnormal proteins. UPP is central to multiple cellular processes, and if defective or imbalanced, it can lead to pathogenesis of a variety of diseases. The attachment of ubiquitin to specific protein substrates can be achieved through the action of E3ubiquitin ligases.

There are over 600 E3ubiquitin ligases which facilitate the ubiquitination of different proteins in vivo, which can be generally divided into four families: HECT-domain E3s, U-box E3s, monomeric RING E3s and multi-subunit E3s. See generally Li et al. (PLOS One, 2008, 3, 1487) titled “Genome-wide and functional annotation of human E3ubiquitin ligases identifies MULAN, a mitochondrial E3that regulates the organelle's dynamics and signaling.”; Bemdsen et al. (Nat. Struct. Mol. Biol., 2014, 21, 301-307) titled “New insights into ubiquitin E3ligase mechanism”; Deshaies et al. (Ann. Rev. Biochem., 2009, 78, 399-434) titled “RING domain E3ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer., 2014, 14, 233-347) titled “Roles of F-box proteins in cancer.”

UPP plays a role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair and biogenesis of organelles.

Aberrations in the process have recently been implicated in the pathogenesis of several diseases, both inherited and acquired. These diseases fall into two major groups: (a) those that result from loss of function with the resultant stabilization of certain proteins, and (b) those that result from gain of function, i.e. abnormal or accelerated degradation of the protein target.

The UPP can be used to induce selective protein degradation, including via use of fusion proteins to ubiquitinate target proteins and synthetic small-molecule probes to induce proteasome-dependent degradation. Bifunctional compounds composed of a target protein-binding ligand and an E3ubiquitin ligase ligand, can induce proteasome-mediated degradation of selected proteins via their recruitment to E3ubiquitin ligase and subsequent ubiquitination. These drug-like molecules offer the possibility of temporal control over protein expression. Such compounds can be capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17 (6): 551-555; Schnnekloth J S Jr., Chembiochem, 2005, 6 (1): 40-46).

The signal Transducer and Activator of Transcription 6 (STAT6) belongs to a family of transcription factors (STAT1, STAT2, STAT3, STAT4, STAT5a, STAT5b and STAT6) which may be structurally and/or functionally related, and which may be involved in mediating signalling from multiple cytokine and/or growth factor receptors.

Without being bound by theory, STAT6can selectively mediate signaling from IL-4 and IL-13 via the IL-4Ra subunit complexing with either the common gamma chain (γc) to form the type I receptor or with the IL-13Rα1 subunit to form a type II receptor. When IL-4 or IL-13 activates the receptor complex, the Janus Kinases (Jak) associated with the cytoplasmic tail of IL-4Ra can be activated and phosphorylate tyrosine residues on the intracellular part of the receptor. This phosphorylation can generate docking site(s) for STAT6, which can bind to the phosphorylated receptor via its Src homology-2 (SH2) domain. This may allow Jak kinases to phosphorylate tyrosine (Y)-641 on STAT6, potentially leading to activation. Activated STAT6may form a homodimer and relocate to the nucleus and activate gene transcription. The genes transcribed by activated STAT6can be cell specific and could in general induce Th2immune responses (Walford and Taylor 2013: STAT6and lung inflammation. JAK-STAT 2:4, e25301; October/November/December 2013; @ 2013 Landes Bioscience).

STAT6 is expressed in numerous cell types including epithelial cells, fibroblasts, and immune cells. Without being bound by theory, inhibition of STAT6activity can inhibit the IL-4 and IL-13 mediated effects in cells, including the differentiation of T-cells into Th2 cells and B-cell class shift into IgE and IgG1 producing cells (Walford). In epidermal keratinocytes, a STAT6inhibitor could inhibit the secretion of pro-inflammatory chemokines and revert the cytokine-induced inhibition of barrier function proteins such as filaggrin (Tollenaire et al 2017: Skin Barrier and Inflammation Genes Associated with Atopic Dermatitis are Regulated by Interleukin-13 and Modulated by Tralokinumab In vitro. Acta Derm Venereol 2021; 101: adv00447.

Antibodies targeting Th2immune responses, such as the IL-4Ra (dupilumab) or IL-13 (tralokinumab, lebrikizumab), have shown efficacy in a number of Th2-driven diseases. Targeting STAT6with a small molecule inhibitor allows for targeting the same pathway by an oral or dermal administration route and may have efficacy in diseases where dupilumab has shown effect. A compound antagonizing STAT6could, therefore, have utility in treating conditions characterized by Th2-mediated inflammation such as atopic dermatitis, prurigo nodularis, Bullous phemphigoid, asthma, chronic rhinosinusitis with nasal polyposis, urticaria (such as chronic spontaneous urticaria), rhinitis, eosinophilic esophagitis, food allergy, diffuse cutaneous systemic sclerosis, alopecia areata and/or COPD.

STAT6 is also involved in differentiation and activity of M2 macrophages, including the tumor-associated macrophages (TAMs) in solid tumors. TAMs protect the tumor from immune attack by inducing a pro-tumor immunosuppressive environment. TAMs may inhibit T-cell proliferation, block migration of CD8 T-cells into the tumor and recruit Tregs into the tumor microenvironment (Karpathiou et al 2021: STAT6: A review of a signaling pathway implicated in various diseases with a special emphasis in its usefulness in pathology. Pathology-Research and Practice 223 (2021) 153477).

In addition, IL-13 may act as a growth factor for some tumors and for some tumors gain-of-function mutations in STAT6have been described as oncogenes (Karpathiou et al 2021: STAT6: A review of a signaling pathway implicated in various diseases with a special emphasis in its usefulness in pathology. Pathology-Research and Practice 223 (2021) 153477).

Together these data suggests that a STAT6 degraders may treat different cancers such as lymphomas. non-small cell lung cancer and solid fibrous cancers either as a stand-alone treatment or in combination with other anticancer drugs such as check point inhibitors.

Although various antibodies against IL-4R or IL-13 are approved for medical use, there are currently no approved, orally available degraders of STAT6.

Therefore, there remains a continuous need to develop degraders of STAT-6, particularly small molecules suitable for oral administration.

In addition, some patients may be treated by topical application of degraders of STAT-6. This can be particularly suitable, for example, for patients with skin lesions that are readily accessible and limited to areas on the body surface. Topical treatment may also be prescribed for certain patients who could benefit from avoiding systemic modulation of the STAT-6 pathway, for example when undergoing treatment for infections or gastrointestinal problems.

### SUMMARY

The inventors have surprisingly found that the novel compounds and salts thereof as described in the present disclosure exhibit modulating effects on the STAT-6 signalling pathway.

For example, the compounds and pharmaceutically acceptable salts thereof as described herein may be beneficial in preventing, treating or ameliorating a variety of diseases which involve up-regulation or de-regulation of STAT-6.

Furthermore, the compounds and pharmaceutically acceptable salts thereof as described herein have advantageous properties such as high metabolic stability, membrane permeability and/or solubility that make them particularly suitable for oral administration.

Moreover, some patients may be treated by topical application of degraders of STAT-6. This can be particularly suitable, for example, for patients with skin lesions that are readily accessible and limited to areas on the body surface. Topical treatment may also be prescribed for certain patients who could benefit from avoiding systemic modulation of the STAT-6 pathway, for example when undergoing treatment for infections or gastrointestinal problems. Thus, some aspects of the present disclosure relate to methods for the topical application of the compounds and salts thereof as described herein.

Accordingly, in some embodiments, provided herein are compounds and pharmaceutically acceptable salts thereof according to formula (I):

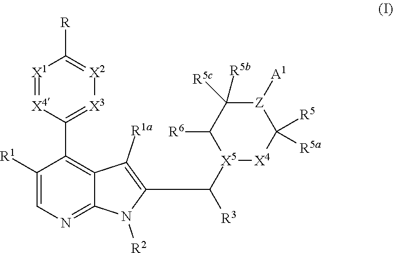

A′ is selected from

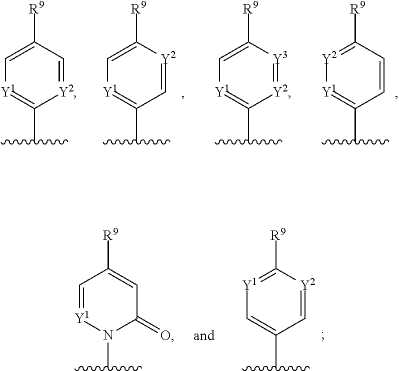

X¹ is selected from CR¹¹ and N; X² is selected from CR¹² and N; X³ is selected from CR¹³ and N, and X⁴ is selected from CR¹⁴ and N, and wherein when X⁴ is N the N may be an N-oxide, or X¹ is CR¹¹ and taken together with R forms a 5-membered heteroaryl ring which is optionally substituted with C(O)CF₃;
X⁵ is selected from CR⁴R⁴ and CO₃
X^{a} is selected from N and oxidized N;
Y¹ is selected from N and CR⁷;
Y² is selected from N and CR⁸;
Y³ is selected from N and CR¹⁷;
Z is selected from N and CR¹⁰;
R is selected from NHR⁰ and —CONHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—R′, —(CH₂)ₙ—CO—C₁₋₄alkyl and —(CH₂)ₙ—CO₂R′, wherein said phenyl is optionally substituted one or more times with substituents independently selected from halogen, C₁₋₄alkyl, halo-C₁₋₄alkyl, C₃₋₄ cycloalkyl, hydroxy, C₁₋₄alkoxy, cyano, —NR′R″, —CONR′R″ and —CO₂R′, wherein R′ and R″ are each independently selected from hydrogen and C₁₋₄alkyl and n is 0, 1 or 2;
R¹ and R^{1a} are independently selected from hydrogen, fluoro and C₁₋₄alkyl;
R² is selected from hydrogen, C₁₋₅alkyl, —SO₂—C₁₋₄alkyl and deuterated C₁₋₄alkyl, wherein said C₁₋₅alkyl may optionally be substituted one or more times with fluoro;
R³ is independently selected from hydrogen, C₁₋₄alkyl, and deuterated C₁₋₄alkyl;
each R⁴ and R⁶ are independently selected from hydrogen, C₁₋₄alkyl, and C₁₋₄alkoxy, wherein said C₁₋₄alkyl and C₁₋₄alkoxy may optionally be substituted with one or more halogen;
R⁵ is selected from hydrogen, halogen, C₁₋₄alkyl and C₁₋₄alkoxy, wherein said C₁₋₄alkyl and C₁₋₄alkoxy may optionally be substituted with one or more halogen;
R¹⁰ is selected from hydrogen and fluoro; or
R⁵ and R¹⁰ together form a bond between the two carbon atoms to which they are attached;
R^{5a} is hydrogen;
or R⁵ and R^{5a} are both fluoro or R⁵ and R^{5a} together with the atom attached thereto form a CO group;
R^{5b} and R^{5c} are each independently selected from hydrogen and fluoro;

R⁷, R⁸, and R¹⁷ are each independently selected from hydrogen, halogen, cyano, C₁₋₄alkyl and C₁₋₄alkoxy, and C₃₋₄cycloalkyl, wherein said C₁₋₄alkyl and C₁₋₄alkoxy may optionally be substituted with one or more halogen;
R⁹ is -A-L-LBM;
R¹¹, R¹², R¹³ and R¹⁴ are each independently selected from hydrogen, halogen, C₁₋₄alkyl, C₃₋₄cycloalkyl, C₁₋₄alkoxy, hydroxy, cyano, —NR′R″, —CONR′R″, —CO₂R′, —CO—CO₂R′, —CO—(CH₂)ₘOH, tetrazolyl and —SO₂—C₁₋₄alkyl, wherein said C₁₋₄alkyl and C₁₋₄alkoxy are optionally substituted one or more times with a substituent independently selected from halogen, hydroxy and C₁₋₄alkoxy, wherein m is 1, 2 or 3; or
R¹¹ and R⁰ together with the atoms attached thereto form a 5-6 membered heteroaromatic or heterocyclic ring containing one or two N atoms, wherein said 5-6 membered heteroaromatic ring or heterocyclic ring containing one or two N atoms is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —COF₃, —NR′R″, —CONR′R″ and —CO₂R′, wherein said C₁₋₄alkyl may optionally be substituted one or more times with a substituent independently selected from halogen and C₁₋₄alkoxy;
A is CO or

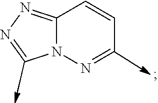

L is selected from:

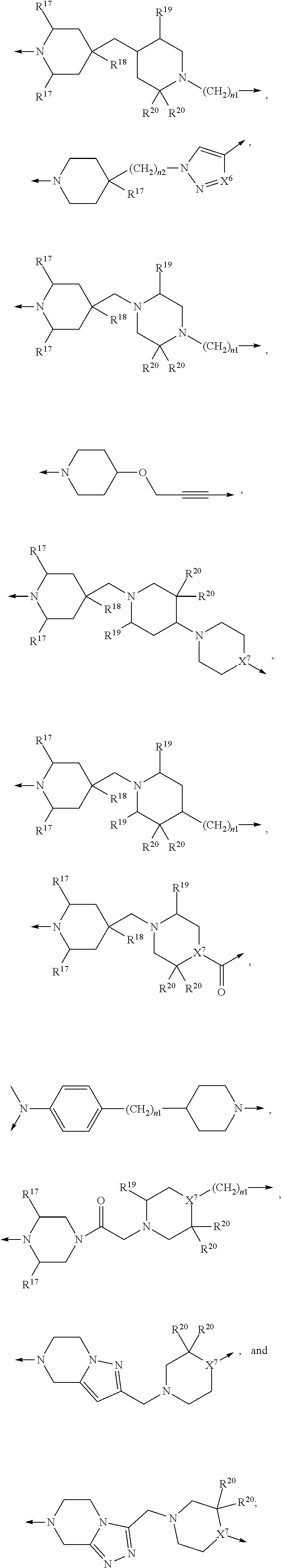

wherein
R¹⁷ is selected from hydrogen and fluoro;
R¹⁸ is selected from hydrogen and fluoro;
R¹⁹ is selected from hydrogen and C₁₋₄alkyl, wherein said C₁₋₄alkyl may optionally be substituted one or more times with fluoro;
R²⁰ is independently selected from hydrogen and fluoro;
n1 is selected from 0 and 1;
n2 is selected from 1 and 2;
X⁶ is CH or N;
X⁷ is selected from N and CH;
and
LBM is selected from

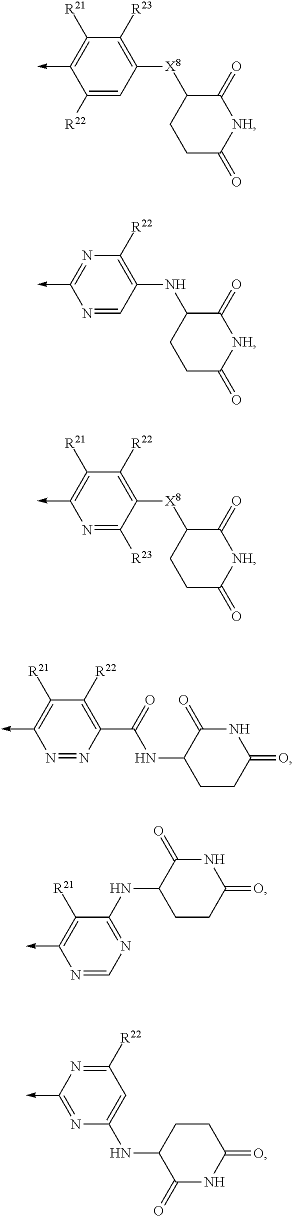

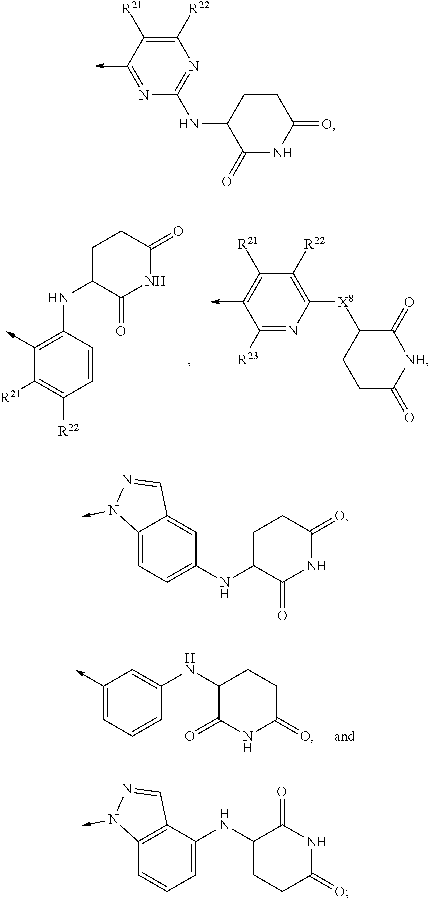

wherein
X⁸ is selected from O, NH, and triazolyl;
R²¹ is selected from hydrogen, fluoro, chloro, cyano, C₁₋₄alkyl and trifluoromethyl;
R²² is selected from hydrogen and fluoro;
R²³ is selected from hydrogen, fluoro and C₁₋₄alkoxy;
or a pharmaceutically acceptable salt or stereoisomer thereof.

Accordingly, in some embodiments, provided herein are compounds and pharmaceutically acceptable salts thereof according to formula (I′):

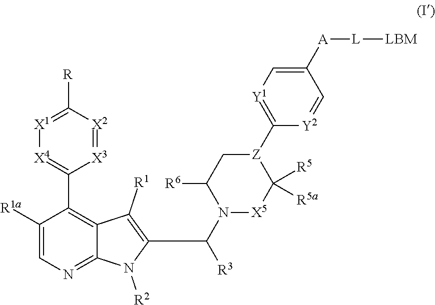

wherein:
X¹ is selected from CR¹¹ and N; X² is selected from CR¹² and N; X³ is selected from CR¹³ and N, and X⁴ is selected from CR¹⁴ and N, and wherein when X⁴ is N the N may be oxidized to a N-oxide;
X⁵ is CO or CR⁴R⁴;
Y¹ is selected from N and CR⁷;
Y² is selected from N and CR⁸;
Z is selected from N and CR¹⁰;
R is NHR⁰;
R⁰ is selected from hydrogen, C₁₋₄alkyl, C₃₋₄cycloalkyl, C₃₋₄cycloalkyl-C₁₋₄alkyl, phenyl-C₁₋₄alkyl, halo-C₁₋₄alkyl, —(CH₂)ₙ—O—R′, —(CH₂), —CO—C₁₋₄alkyl and —(CH₂)ₙ—CO₂R′,
wherein said phenyl is optionally substituted one or more times with substituents independently selected from halogen, C₁₋₄alkyl, halo-C₁₋₄alkyl, C₃₋₄ cycloalkyl, hydroxy, C₁₋₄alkoxy, cyano, —NR′R″, —CON R′R″, and —CO₂R′, wherein R′ and R″ are each independently selected from hydrogen and C₁₋₄alkyl and n is 0, 1, or 2;
R¹ and R^{1a} are independently selected from hydrogen, fluoro, and C₁₋₄alkyl;
R² is selected from hydrogen, C₁₋₅alkyl, and deuterated C₁₋₄alkyl, wherein said C₁₋₅alkyl may optionally be substituted one or more times with fluoro;
R³ is selected from hydrogen, C₁₋₄alkyl and deuterated C₁₋₄alkyl;
each of R⁴ and R⁶ are independently selected from hydrogen, C₁₋₄alkyl, and C₁₋₄alkoxy, wherein said C₁₋₄alkyl and C₁₋₄alkoxy may optionally be substituted with one or more halogen;
R⁵ is selected from hydrogen, fluoro, and C₁₋₄alkyl; wherein said C₁₋₄alkyl may optionally be substituted with one or more halogen, and R^{5a} and R¹⁰ is hydrogen; or
R⁵ and R^{5a} are both selected from fluoro and R¹⁰ is hydrogen; or
R⁵ and R¹⁰ together form a bond between the two carbon atoms to which they are attached, and R^{5a} is hydrogen;
R⁷ and R⁸ are independently selected from hydrogen, halogen, C₁₋₄alkyl, and C₁₋₄alkoxy, wherein said C₁₋₄alkyl and C₁₋₄alkoxy may optionally be substituted with one or more halogen;
R¹¹, R¹², R¹³ and R¹⁴ are independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′, —CO—(CH₂)ₘOH, tetrazolyl and —SO₂—C₁₋₄alkyl, wherein said C₁₋₄alkyl and C₁₋₄alkoxy is optionally substituted one or more times with a substituent independently selected from halogen, hydroxy, and C₁₋₄alkoxy, wherein m is 1, 2 or 3;
R¹¹ and R⁰ may together form a 5-6 membered heteroaromatic ring or 5-6 membered heterocyclic ring containing one or two N atoms, wherein said 5-6 membered heteroaromatic ring or 5-6 membered heterocyclic ring containing one or two N atoms is optionally be substituted one or more times with a substituent independently selected from halogen, C₁₋₄alkyl, halo-C₁₋₄alkyl, C₃₋₄ cycloalkyl, hydroxy, C₁₋₄alkoxy, cyano, —COF₃, —NR′R″, —CONR′R″, and —CO₂R′, wherein said C₁₋₄alkyl may optionally be substituted one or more times with a substituent independently selected from halogen and C₁₋₄alkoxy;
A is CO or

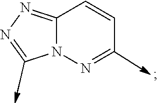

L is selected from:

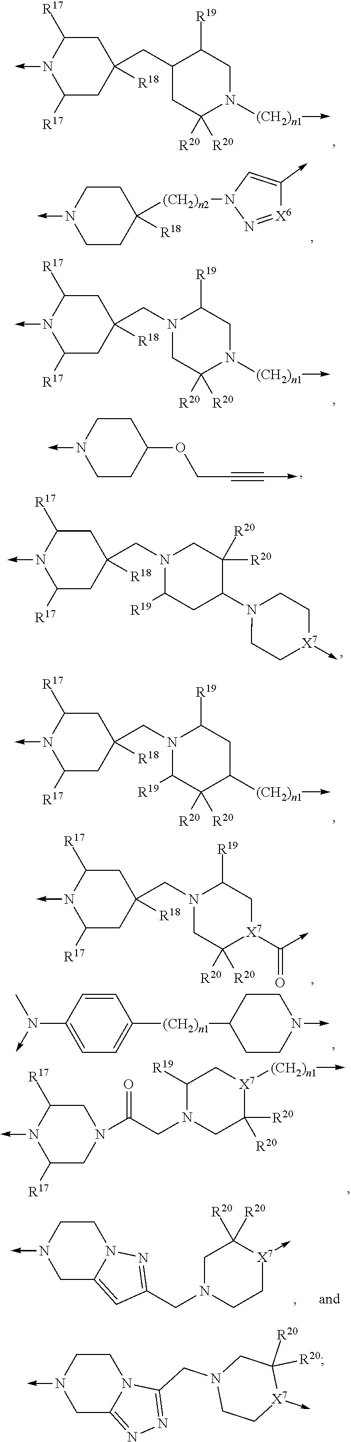

wherein
R¹⁷ is selected from hydrogen and fluoro;
R¹⁸ is selected from hydrogen and fluoro;
R¹⁹ is selected from hydrogen and C₁₋₄alkyl, wherein said C₁₋₄alkyl may optionally be substituted one or more times with fluoro;
R²⁰ is independently selected from hydrogen and fluoro;
n1 is selected from 0 and 1;
n2 is selected from 1 and 2;
X⁶ is CH or N;
X⁷ is selected from N and CH;
and
LBM is selected from

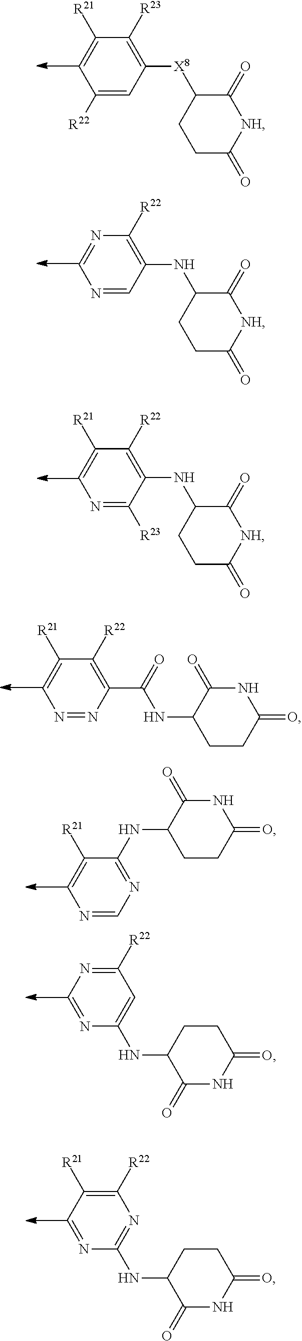

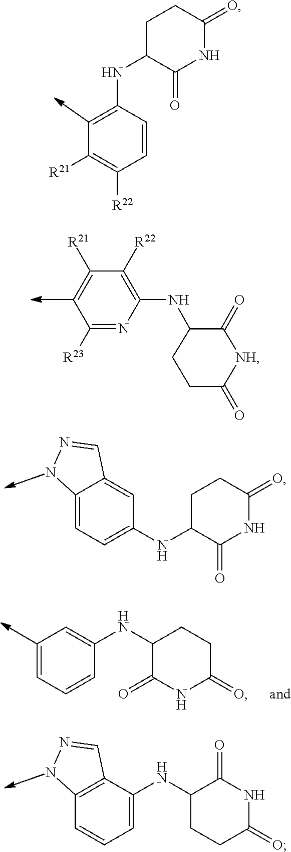

wherein
X⁸ is selected from O, NH, and triazolyl;
R²¹ is selected from hydrogen, fluoro, chloro, cyano, C₁₋₄alkyl and trifluoromethyl;
R²² is selected from hydrogen and fluoro;
R²³ is selected from hydrogen, fluoro and C₁₋₄alkoxy;
or a pharmaceutically acceptable salt or stereoisomer thereof.

In another embodiment, the present disclosure provides a pharmaceutical composition comprising a compound of the present disclosure, or a pharmaceutically acceptable salt or stereoisomer thereof, and a pharmaceutically acceptable excipient.

In another embodiment, the present disclosure provides a method of treating an immune mediated disease or condition in a subject in need thereof, the method comprising administering to the subject a therapeutically effective amount of a compound of the present disclosure, or a pharmaceutically acceptable salt or stereoisomer thereof, or a pharmaceutical composition of the present disclosure.

In another embodiment, the present disclosure provides a method of modulating a signal transducer and activator of transcription 6 (STAT6) protein activity in a subject in need thereof, the method comprising administering to the subject a therapeutically effective amount of a compound of the present disclosure, or a pharmaceutically acceptable salt or stereoisomer thereof, or a pharmaceutical composition of the present disclosure.

In another embodiment, the present disclosure provides a method of treating a disease or condition mediated by a signal transducer and activator of transcription 6 (STAT6) protein activity in a subject in need thereof, the method comprising administering to the subject a therapeutically effective amount of a compound of the present disclosure, or a pharmaceutically acceptable salt or stereoisomer thereof, or a pharmaceutical composition of the present disclosure.

In another embodiment, the present disclosure provides a method of treating a disease or condition mediated by interleukin 4 (IL-4) or interleukin 3 (IL-3) in a subject in need thereof, the method comprising administering to the subject a therapeutically effective amount of a compound of the present disclosure, or a pharmaceutically acceptable salt thereof or stereoisomer, or a pharmaceutical composition of the present disclosure.

In an embodiment the disclosure provides a method of preventing, treating or ameliorating a diseases characterized by Th2-mediated inflammation.

In another embodiment, the present disclosure provides a method for manufacturing a medicament for treating a disease or condition mediated by a signal transducer and activator of transcription 6 (STAT6) protein activity in a subject in need thereof, characterized in that a compound of the present disclosure, or a pharmaceutically acceptable salt or stereoisomer thereof, is used.

In another embodiment, the present disclosure provides a method for manufacturing a medicament for the treatment of a disease or condition mediated by a signal transducer and activator of transcription 6 (STAT6) protein in a subject.

In some embodiments, the present disclosure provides a compound of the present disclosure, or a pharmaceutically acceptable salt or stereoisomer thereof, for use in treating a disease or condition mediated by a signal transducer and activator of transcription 6 (STAT6) protein in a subject in need thereof.

In some embodiments, the present disclosure provides the compound of the present disclosure, or a pharmaceutically acceptable salt thereof or stereoisomer, for use in therapy.

### BRIEF DESCRIPTION OF THE DRAWINGS

FIG. 1 shows the sensorgrams for Examples 2a1, 2t1, 2m1, and 2a2 are shown below (where RU is plotted vs. time in seconds; dotted lines/points were excluded from the analysis due to effects from non-specific binding)

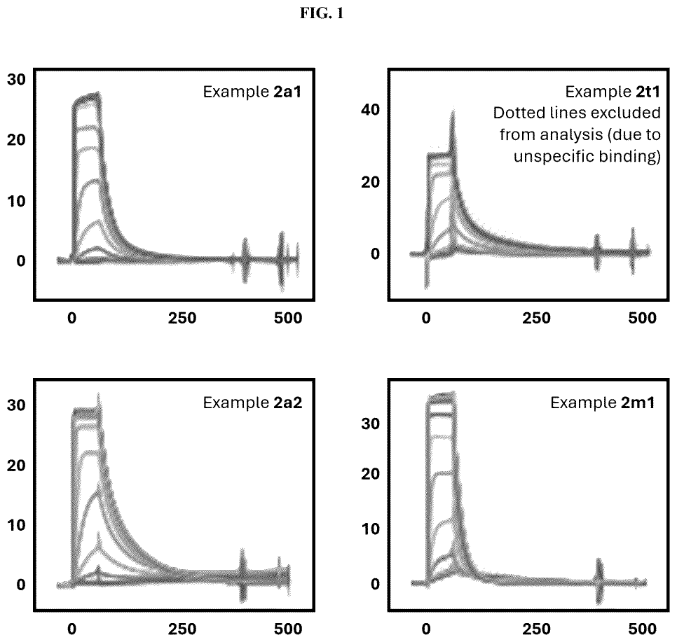

FIG. 2 shows the dose-response curves for Examples 2a2, 2t1, 2 h3, and 2r1 are shown below (where percent degradation is plotted vs. test compound concentration in M on a log-scale).

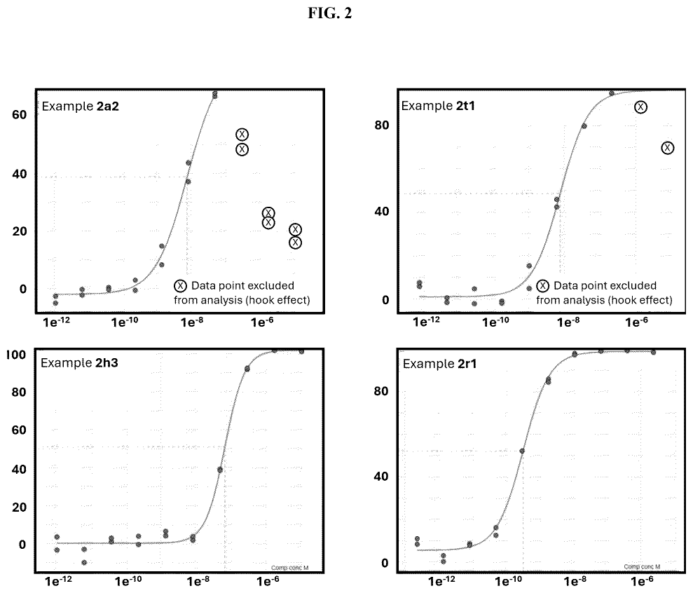

FIG. 3 shows the data from the human whole blood eotaxin-3 assay is shown below for Examples 2q11, 2110, and 2c49 (where the effect in % is plotted against the test concentration in M on a log-scale).

### DEFINITIONS

Whenever the compound of formula (I) is mentioned herein it should be understood that the compound of formula (I′), (II), (III), (I-b), (II-b), and (III-b), and other subformulas described herein, are subgroups of the compound of formula (I) and that a statement related to the compound of formula (I) relates equally well to its subgroups.

The prefix “Cᵤ₋ᵥ” indicates that the following group has from u to v carbon atoms. For example, “C14 alkyl” indicates that the alkyl group has from 1 to 4 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

The term “C₁₋₄alkyl” is used herein to refer to hydrocarbon radical obtained when one hydrogen atom is removed from a branched or linear hydrocarbon. Said alkyl comprises (1-4) carbon atoms, 1-3 carbon atoms, 2-3 carbon atoms or 1-2 carbon atoms. The term includes the subclasses normal alkyl (n-alkyl), secondary and tertiary alkyl, such as methyl, ethyl, n-propyl, isopropyl, n-butyl, isobutyl, sec-butyl, and tert-butyl.

The term “(C₁-C₄) alkoxy” is used herein to refer to a radical of the formula —OR^{a}, wherein R^{a} is (C₁-C₄) alkyl as indicated herein, wherein the (C₁-C₄) alkyl group is appended to the parent molecular moiety through an oxygen atom, e.g. methoxy (—OCH₃), and ethoxy (—OCH₂CH₃).

The term “cyano” is used herein to refer to a-CN group attached to the parent molecular moiety through the carbon atom.

The term “(C₃-C₄) cycloalkyl” is used herein to refer to a saturated (C₃-C₄) cycloalkane hydrocarbon radical, comprising 3-4 carbon atoms, e.g. cyclopropyl or cyclobutyl.

The term “halogen” or “halo” is used herein to refer to chloro, bromo, fluoro, or iodo. In some embodiments, halogen is chloro, bromo, or fluoro.

“Aromatic ring” or “aryl” refers to an aromatic carbocyclic group having a single ring (e.g., monocyclic) or multiple rings (e.g., bicyclic or tricyclic) including fused systems. An aryl may have 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Heteroaromatic ring” or “heteroaryl” refers to an aromatic group having a single ring, multiple rings or multiple fused rings, with one or more ring heteroatoms independently selected from nitrogen, oxygen, and sulfur unless specified otherwise. A heteroaryl may include 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6] imidazo [1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, thiophenyl (i.e., thienyl), triazolyl, tetrazolyl, and triazinyl. Examples of the fused-heteroaryl rings include, but are not limited to, benzo[d] thiazolyl, quinolinyl, isoquinolinyl, benzo[b]thiophenyl, indazolyl, benzo[d] imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclic ring” or “heterocyclyl” refers to a saturated or partially unsaturated cyclic alkyl group, with one or more ring heteroatoms independently selected from nitrogen, oxygen, and sulfur, unless specified otherwise. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (═O) or N-oxide (—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. A heterocyclyl may have 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b] [1,4] dioxepinyl, 1,4-benzodioxanyl, benzopyranyl, benzodioxinyl, benzopyranonyl, benzofuranonyl, dioxolanyl, dihydropyranyl, hydropyranyl, thienyl [1,3] dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiomorpholinyl, thiamorpholinyl, 1-oxo-thiomorpholinyl, and 1,1-dioxo-thiomorpholinyl.

The term “halo-C₁₋₄alkyl” or “halo-C₁₋₄alkoxy” is used herein to refer to an “C₁₋₄alkyl” or “C₁₋₄alkoxy” group respectively as defined above in which one or more hydrogen atoms have been replaced by halogen (e.g. fluorine). Examples of “halo-C₁₋₄alkyl” or “halo-C₁₋₄alkoxy” include —CHF₂, —CF₃, —CH₂CF₃, —CF₂CF₃ or —OCF₃.

The term “deuterated C₁₋₄alkyl” is used herein to refer to an “C₁₋₄alkyl” group in which one or more hydrogen atoms have been replaced by deuterium. Examples of “deuterated C₁₋₄alkyl” include —CH₂D₂, -CHD₂ or -CD₃.

The term “C₁₋₄alkoxyl-C₁₋₄alkyl” is used herein the refer to “C₁₋₄alkyl” group in which one hydrogen atom have been replaced by (C₁-C₄) alkoxy. Examples of “C₁₋₄alkoxyl-C₁₋₄alkyl” include methoxymethyl or methoxyethyl.

The term “(C₃-C₄) cycloalkyl-C₁₋₄alkyl” is used herein the refer to “C₁₋₄alkyl” group in which one hydrogen atom have been replaced by (C₃-C₄) cycloalkyl. Examples of “(C₃-C₄) cycloalkyl-C₁₋₄alkyl” include cyclopropylmethyl.

The term “phenyl-C₁₋₄alkyl” is used herein the refer to “C₁₋₄alkyl” group in which one hydrogen atom have been replaced by phenyl. Examples of “phenyl-C₁₋₄alkyl” include benzyl.

If substituents are described as being independently selected from a group, each substituent is selected independent of the other. Each substituent may therefore be identical or different from the other substituent(s).

The term “optionally substituted” means “unsubstituted or substituted.” In some embodiments, formulas described herein encompasses compounds containing the specified optional substituent(s) as well as compounds that do not contain the optional substituent(s).

In certain embodiments, as used herein, the phrase “one or more” refers to one to five. In certain embodiments, as used herein, the phrase “one or more” refers to one to three.

As used herein whenever a molecular drawing of a substituent contains an arrow—the arrow indicates the bond attaching the substituent to the rest of the molecule.

The term “pharmaceutically acceptable salt” is intended to indicate non-toxic salts including a free base form of a compound that possesses the desired pharmacological activity of the free base. These salts may be derived from an appropriate basic moiety, with a suitable inorganic or organic acid, such as hydrochloric, hydrobromic, hydroiodic, sulfuric, nitric, phosphoric, formic, acetic, 2,2-dichloroacetic, adipic, ascorbic, L-aspartic, L-glutamic, galactaric, lactic, maleic, L-malic, phthalic, citric, propionic, benzoic, glutaric, gluconic, D-glucuronic, methanesulfonic, salicylic, succinic, malonic, tartaric, benzenesulfonic, ethane-1,2-disulfonic, 2-hydroxyethanesulfonic acid, toluenesulfonic, sulfamic or fumaric acid.

Pharmaceutically acceptable salts of compounds of formula (I) comprising an acidic moiety may also be prepared by reaction with a suitable base such as sodium hydroxide, potassium hydroxide, magnesium hydroxide, calcium hydroxide, zinc hydroxide, barium hydroxide, ammonia or the like, or suitable non-toxic amines, such as lower alkylamines (such as diethylamine, tetraalkylammonium hydroxide), hydroxy-lower alkylamines (such as diethanolamine, 2-(diethylamino)-ethanol, ethanolamine, triethanolamine, tromethamine, deanol), cycloalkylamines, ethylene diamine, or benzylamines, (such as benethamine and benzathine), betaine, choline hydroxide, N-methyl-glucamine, hydrabamine, 1H-imidazole, 4-(2-hydroxyethyl)-morpholine, piperazine, 1-(2-hydroxyethyl)-pyrrolidine, L-arginine or L-lysine. Further examples of pharmaceutical acceptable salts are listed in Berge, S. M.; J. Pharm. Sci.; (1977), 66 (1), 1-19, and Stahl, P. H. and in Wermuth, C. G, Handbook of Pharmaceutical Salts, Properties, Selection and Use, 2nd Edition, Wiley-VCH, 2011 both of which are incorporated herein by reference.

Compounds of the disclosure containing an amine function may also form N-oxides.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

The term “solvate” is intended to indicate a species formed by interaction between a compound, e.g. a compound of formula (I) or a pharmaceutically acceptable salt or stereoisomer thereof, and a solvent, e.g. alcohol, glycerol or water, wherein said species are in a crystalline form. When water is the solvent, said species is referred to as a hydrate.

The term “treatment” as used herein means the management and care of a patient for the purpose of combating a disease, disorder or condition. The term is intended to include the delaying of the progression of the disease, disorder or condition, the amelioration, alleviation or relief of symptoms and complications, and/or the cure or elimination of the disease, disorder or condition. The term may also include prevention of the condition, wherein prevention is to be understood as the management and care of a patient for the purpose of combating the disease, condition or disorder and includes the administration of the active compounds to prevent the onset of the symptoms or complications. Nonetheless, prophylactic (preventive) and therapeutic (curative) treatments are two separate aspects.

“Administering” refers to oral administration, administration as a suppository, topical contact (e.g., transdermal), parenteral, intravenous, intraperitoneal, intramuscular, intralesional, intranasal, inhaled, intradermal, and/or subcutaneous administration, intrathecal administration, and/or the implantation of a slow-release device e.g., a mini-osmotic pump, to the subject. The administration can be carried out according to a schedule specifying frequency of administration, dose for administration, and other factors.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

### Compounds

Provided herein are compounds that modulate the activity of STAT6.

In one embodiments, provided is a compound of formula (I):
A′ is selected from

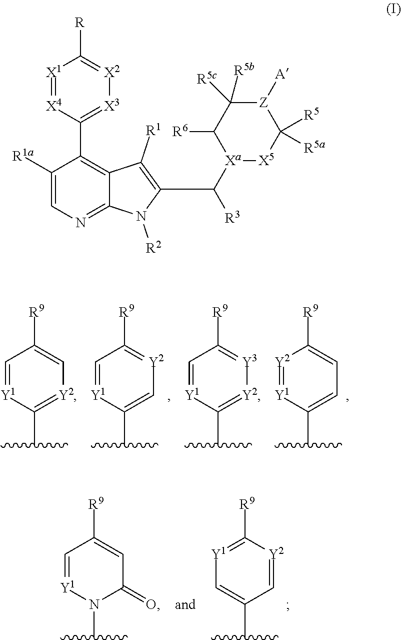

X¹ is selected from CR¹¹ and N; X² is selected from CR¹² and N; X³ is selected from CR¹³ and N, and X⁴ is selected from CR¹⁴ and N, and wherein when X⁴ is N the N may be an N-oxide, or X¹ is CR¹¹ and taken together with R forms a 5-membered heteroaryl ring which is optionally substituted with C(O)CF₃;
X⁵ is selected from CR⁴R⁴ and CO₃
X^{a} is selected from N and oxidized N;
Y¹ is selected from N and CR⁷;
Y² is selected from N and CR⁸;
Y³ is selected from N and CR¹⁷;
Z is selected from N and CR¹⁰;
R is selected from NHR⁰ and —CONHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—R′, —(CH₂)ₙ—CO—C₁₋₄alkyl and —(CH₂)—CO₂R′, wherein said phenyl is optionally substituted one or more times with substituents independently selected from halogen, C₁₋₄alkyl, halo-C₁₋₄alkyl, C₃₋₄ cycloalkyl, hydroxy, C₁₋₄alkoxy, cyano, —NR′R″, —CONR′R″ and —CO₂R′, wherein R′ and R″ are each independently selected from hydrogen and C₁₋₄alkyl and n is 0, 1 or 2;
R¹ and R^{1a} are independently selected from hydrogen, fluoro and C₁₋₄alkyl;
R² is selected from hydrogen, C₁₋₅alkyl, —SO₂—C₁₋₄alkyl and deuterated C₁₋₄alkyl, wherein said C₁₋₅alkyl may optionally be substituted one or more times with fluoro;
R³ is independently selected from hydrogen, C₁₋₄alkyl, and deuterated C₁₋₄alkyl;
each R⁴ and R⁶ are independently selected from hydrogen, C₁₋₄alkyl, and C₁₋₄alkoxy, wherein said C₁₋₄alkyl and C₁₋₄alkoxy may optionally be substituted with one or more halogen;
R⁵ is selected from hydrogen, halogen, C₁₋₄alkyl and C₁₋₄alkoxy, wherein said C₁₋₄alkyl and C₁₋₄alkoxy may optionally be substituted with one or more halogen;
R¹⁰ is selected from hydrogen and fluoro; or
R⁵ and R¹⁰ together form a bond between the two carbon atoms to which they are attached;
R^{5a} is hydrogen;
or R⁵ and R^{5a} are both fluoro or R⁵ and R^{5a} together with the atom attached thereto form a CO group;
R^{5b} and R^{5c} are each independently selected from hydrogen and fluoro;
R⁷, R⁸, and R¹⁷ are each independently selected from hydrogen, halogen, cyano, C₁₋₄alkyl and C₁₋₄alkoxy, and C₃₋₄cycloalkyl, wherein said C₁₋₄alkyl and C₁₋₄alkoxy may optionally be substituted with one or more halogen;
R⁹ is -A-L-LBM;
R¹¹, R¹², R¹³ and R¹⁴ are each independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′, —CO—(CH₂)ₘOH, tetrazolyl and —SO₂—C₁₋₄alkyl, wherein said C₁₋₄alkyl and C₁₋₄alkoxy are optionally substituted one or more times with a substituent independently selected from halogen, hydroxy and C₁₋₄alkoxy, wherein m is 1, 2 or 3; or
R¹¹ and R⁰ together with the atoms attached thereto form a 5-6 membered heteroaromatic or heterocyclic ring containing one or two N atoms, wherein said 5-6 membered heteroaromatic ring or heterocyclic ring containing one or two N atoms is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —COF₃, —NR′R″, —CONR′R″ and —CO₂R′, wherein said C₁₋₄alkyl may optionally be substituted one or more times with a substituent independently selected from halogen and C₁₋₄alkoxy;
A is CO or

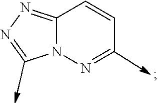

L is selected from:

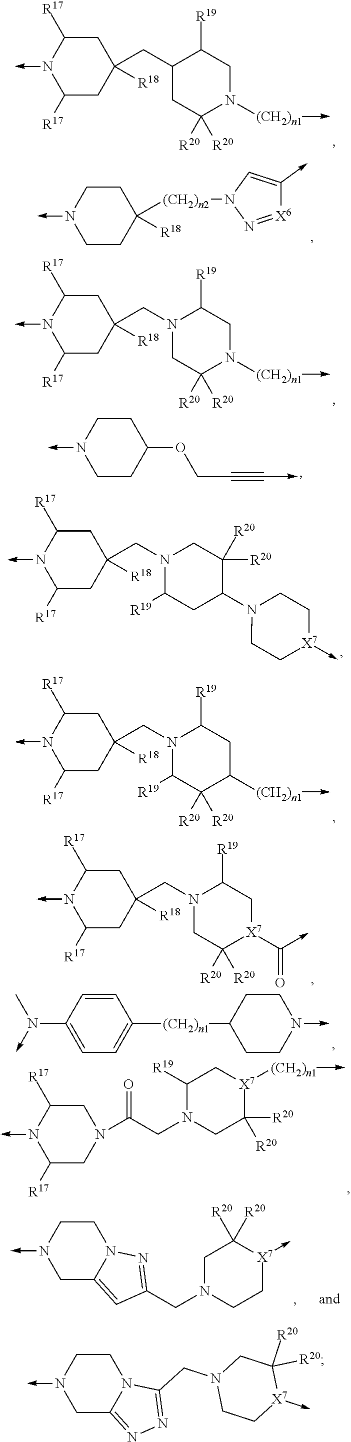

wherein
R¹⁷ is selected from hydrogen and fluoro;
R¹⁸ is selected from hydrogen and fluoro;
R¹⁹ is selected from hydrogen and C₁₋₄alkyl, wherein said C₁₋₄alkyl may optionally be substituted one or more times with fluoro;
R²⁰ is independently selected from hydrogen and fluoro;
n1 is selected from 0 and 1;
n2 is selected from 1 and 2;
X⁶ is CH or N;
X⁷ is selected from N and CH;
and
LBM is selected from

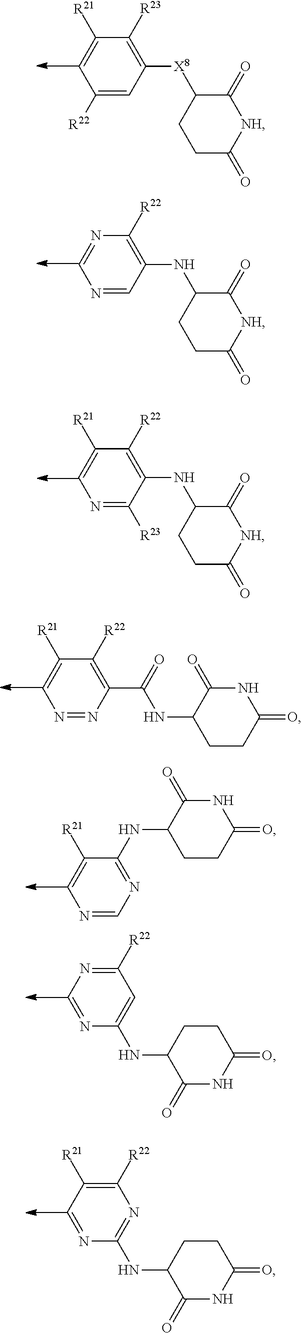

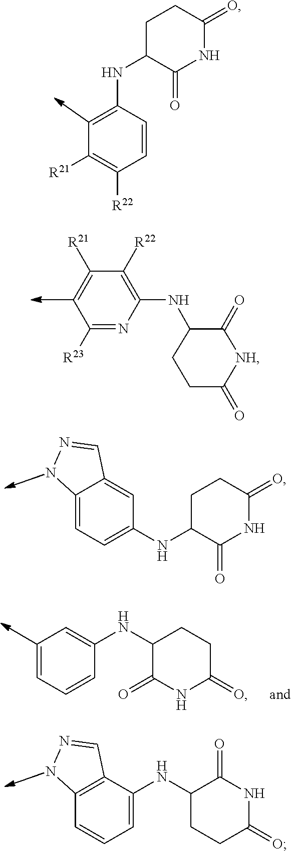

wherein
X⁸ is selected from O, NH, and triazolyl;
R²¹ is selected from hydrogen, fluoro, chloro, cyano, C₁₋₄alkyl and trifluoromethyl;
R² is selected from hydrogen and fluoro;
R²³ is selected from hydrogen, fluoro and C₁₋₄alkoxy;
or a pharmaceutically acceptable salt or stereoisomer thereof.

In one embodiment, provided is a compound of formula (I′):

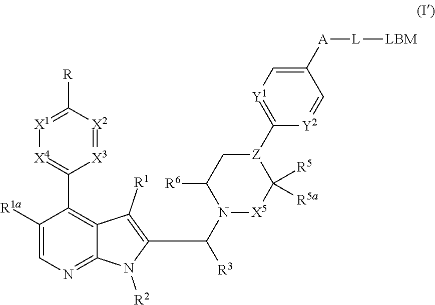

wherein:
X¹ is selected from CR¹¹ and N; X² is selected from CR¹² and N; X³ is selected from CR¹³ and N, and X⁴ is selected from CR¹⁴ and N, and wherein when X⁴ is N the N may be oxidized to a N-oxide;
X⁵ is CO or CR⁴R⁴;
Y¹ is selected from N and CR⁷;
Y² is selected from N and CR⁸;
Z is selected from N and CR¹⁰;
R is NHR⁰;
R⁰ is selected from hydrogen, C₁₋₄alkyl, C₃₋₄cycloalkyl, C₃₋₄cycloalkyl-C₁₋₄alkyl, phenyl-C₁₋₄alkyl, halo-C₁₋₄alkyl, —(CH₂)ₙ—O—R′, —(CH₂)ₙ—CO—C₁₋₄alkyl and —(CH₂)ₙ—CO₂R′,
wherein said phenyl is optionally substituted one or more times with substituents independently selected from halogen, C₁₋₄alkyl, halo-C₁₋₄alkyl, C₃₋₄ cycloalkyl, hydroxy, C₁₋₄alkoxy, cyano, —NR′R″, —CON R′R″, and —CO₂R′, wherein R′ and R″ are each independently selected from hydrogen and C₁₋₄alkyl and n is 0, 1, or 2;
R¹ and R^{1a} are independently selected from hydrogen, fluoro, and C₁₋₄alkyl;
R² is selected from hydrogen, C₁₋₅alkyl, and deuterated C₁₋₄alkyl, wherein said C₁₋₅alkyl may optionally be substituted one or more times with fluoro;
R³ is selected from hydrogen, C₁₋₄alkyl and deuterated C₁₋₄alkyl;
each of R⁴ and R⁶ are independently selected from hydrogen, C₁₋₄alkyl, and C₁₋₄alkoxy, wherein said C₁₋₄alkyl and C₁₋₄alkoxy may optionally be substituted with one or more halogen;
R⁵ is selected from hydrogen, fluoro, and C₁₋₄alkyl; wherein said C₁₋₄alkyl may optionally be substituted with one or more halogen, and R^{5a} and R¹⁰ is hydrogen; or
R⁵ and R^{5a} are both selected from fluoro and R¹⁰ is hydrogen; or
R⁵ and R¹⁰ together form a bond between the two carbon atoms to which they are attached, and R^{5a} is hydrogen;
R⁷ and R⁸ are independently selected from hydrogen, halogen, C₁₋₄alkyl, and C₁₋₄alkoxy, wherein said C₁₋₄alkyl and C₁₋₄alkoxy may optionally be substituted with one or more halogen;
R¹¹, R¹², R¹³ and R¹⁴ are independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′, —CO—(CH₂)ₘOH, tetrazolyl and —SO₂—C₁₋₄alkyl, wherein said C₁₋₄alkyl and C₁₋₄alkoxy is optionally substituted one or more times with a substituent independently selected from halogen, hydroxy, and C₁₋₄alkoxy, wherein m is 1, 2 or 3;
R¹¹ and R⁰ may together form a 5-6 membered heteroaromatic ring or 5-6 membered heterocyclic ring containing one or two N atoms, wherein said 5-6 membered heteroaromatic ring or 5-6 membered heterocyclic ring containing one or two N atoms is optionally be substituted one or more times with a substituent independently selected from halogen, C₁₋₄alkyl, halo-C₁₋₄alkyl, C₃₋₄ cycloalkyl, hydroxy, C₁₋₄alkoxy, cyano, —COF₃, —NR′R″, —CONR′R″, and —CO₂R′, wherein said C₁₋₄alkyl may optionally be substituted one or more times with a substituent independently selected from halogen and C₁₋₄alkoxy;
A is CO or

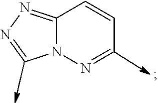

L is selected from:

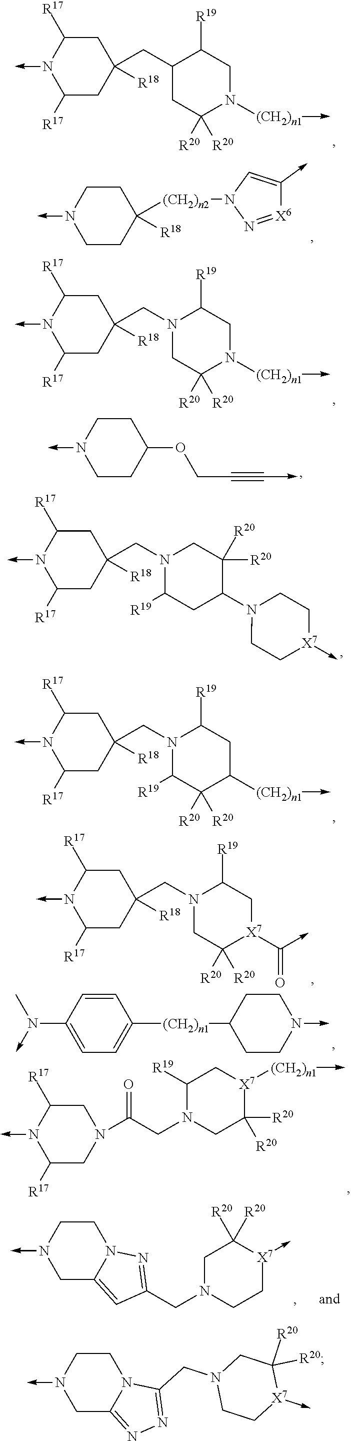

wherein
R¹⁷ is selected from hydrogen and fluoro;
R¹⁸ is selected from hydrogen and fluoro;
R¹⁹ is selected from hydrogen and C₁₋₄alkyl, wherein said C₁₋₄alkyl may optionally be substituted one or more times with fluoro;
R²⁰ is independently selected from hydrogen and fluoro;
n1 is selected from 0 and 1;
n2 is selected from 1 and 2;
X⁶ is CH or N;
X⁷ is selected from N and CH;
and
LBM is selected from

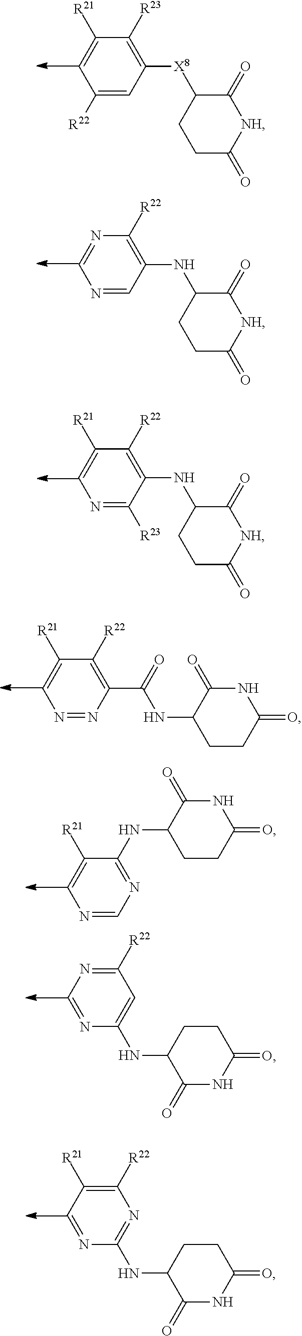

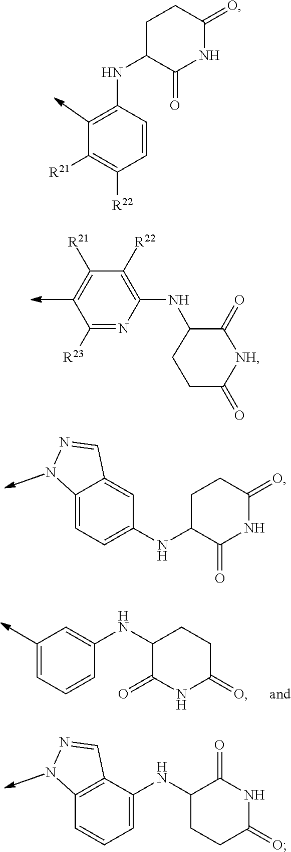

wherein
X⁸ is selected from O, NH, and triazolyl;
R²¹ is selected from hydrogen, fluoro, chloro, cyano, C₁₋₄alkyl and trifluoromethyl;
R²² is selected from hydrogen and fluoro;
R²³ is selected from hydrogen, fluoro and C₁₋₄alkoxy;
or a pharmaceutically acceptable salt or stereoisomer thereof.

In one embodiment is provided a compound of formula (II):

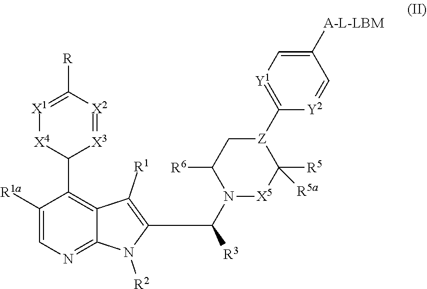

wherein X¹, X², X³, X⁴, X⁵, Y¹, Y², Z, R, R¹, R¹, R², R⁵, R^{5a}, R⁶, A, L and LBM are as defined herein and R³ is C₁₋₄alkyl, or a pharmaceutically acceptable salt or stereoisomer thereof.

In one embodiment L is:

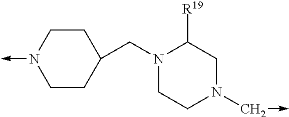

and wherein R¹⁹ is as defined herein. In one embodiment R¹⁹ is hydrogen.

In one embodiment L is:

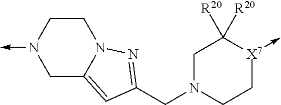

wherein R²⁰ and X⁷ are as defined herein. In some embodiments, X⁷ is CH. In some embodiments each R²⁰ is hydrogen.

In one embodiment LBM is:

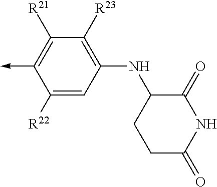

wherein R²¹, R²² and R²³ are as defined above.

In one embodiment, A-L-LBM is

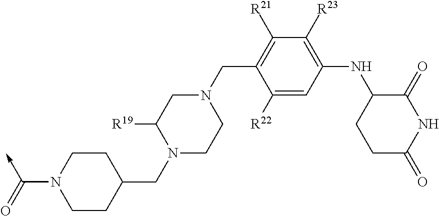

wherein R¹⁹, R²¹, R²² and R²³ are as defined above.

In one embodiment, R²¹ is fluoro and R²² and R²³ is hydrogen.

In one embodiment, X¹ is N, X² is CR¹², X³ is CR¹³ and X⁴ is CR¹⁴.

In one embodiment, R is NHR⁰ and R⁰ is selected from hydrogen and C₁₋₄alkyl.

In one embodiment, X¹ is CH, X² is CH, X³ is CH and X⁴ is CH and R⁰ and R¹¹ together form a pyrazole ring or imidazole ring; where said pyrazole or imidazole ring may optionally be substituted by a substituent independently selected from halogen, C₁₋₄alkyl, halo-C₁₋₄alkyl, C₃₋₄ cycloalkyl, hydroxy, C₁₋₄alkoxy, cyano, —COF₃, —NR′R″, —CONR′R″ and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl. In one embodiment R⁰ and R¹¹, together with the atoms attached thereto a imidazolidinone ring optionally substituted with C₁₄ alkyl.

In a further embodiment the disclosure provides a compound as above wherein said pyrazole or imidazole is either unsubstituted or substituted by C₁₋₄alkoxy-C₁₋₄alkyl.

In a further embodiment the disclosure provides to a compound as above wherein Z is CR¹⁰ and R¹⁰ is hydrogen.

In a further embodiment the disclosure provides to a compound as above wherein Y¹ is CR⁷ and Y² is CR⁸.

In a further embodiment the disclosure provides to a compound as above wherein or both of R⁷ and R⁸ is hydrogen.

In a further embodiment the disclosure provides to a compound as above wherein R¹ is hydrogen an R² and R³ is methyl.

In some embodiments, at least one of X¹, X², X³, and X⁴ is N.

In some embodiments, one and only one of X¹, X², X³, and X⁴ is N.

In some embodiments, X¹ is N, X² is CR¹², X³ is CR¹³, and X⁴ is CR¹⁴.

In some embodiments, X¹ is N, X² is CR¹², X³ is CR¹³, and X⁴ is N.

In some embodiments, each of R¹², R¹³, and R¹⁴, when present, is independently selected from hydrogen, halogen, C₁₋₄alkyl, C₁₋₄alkoxy, and cyano.

In some embodiments, R¹² is hydrogen, R¹³ is hydrogen, and R¹⁴ is hydrogen.

In some embodiments, X¹ is CR¹¹ or N; X² is CR¹²; X³ is CR¹³; X⁴ is CR¹⁴; and R¹¹, R¹², R¹³, and R¹⁴ are each independently selected from hydrogen, halogen, C₁₋₄alkyl, C₁₋₄alkoxy, and cyano.

In some embodiments, X¹ is N; X² is CR¹²; X³ is CR¹³; X⁴ is CR¹⁴; and R¹², R¹³, and R¹⁴ are each independently selected from hydrogen, halogen, C₁₋₄alkyl, and cyano.

In some embodiments, X¹ is CR¹¹ or N; X² is CR¹²; X³ is CR¹³; X⁴ is CR¹⁴; and R¹¹, R¹², R¹³, and R¹⁴ are each independently selected from hydrogen, halogen, C₁₋₄alkyl, C₁₋₄alkoxy, and cyano; or R⁰ and R¹¹ together with the atoms attached thereto form a 5-6 membered heteroaromatic or heterocyclic ring containing one or two N atoms, wherein said 5-6 membered heteroaromatic or heterocyclic ring containing one or two N atoms may optionally be substituted one or more times with a substituent independently selected from halogen, C₁₋₄alkyl, halo-C₁₋₄alkyl, C₃₋₄ cycloalkyl, hydroxy, C₁₋₄alkoxy, cyano, —NR′R″, —CONR′R″, and —CO₂R′; wherein said C₁₋₄alkyl may optionally be substituted one or more times with a substituent independently selected from halogen and C₁₋₄alkoxy; and R′ and R″ are each independently selected from hydrogen and C₁₋₄alkyl.

In some embodiments, X¹ is CR¹¹; X² is CR¹²; X³ is CR¹³; X⁴ is CR¹⁴; and R⁰ and R¹¹ together with the atoms attached thereto form a 5-6 membered heteroaromatic or heterocyclic ring containing one or two N atoms, wherein said 5-6 membered heteroaromatic or heterocyclic ring containing one or two N atoms may optionally be substituted one or more times with a substituent independently selected from halogen, C₁₋₄alkyl, halo-C₁₋₄alkyl, C₃₋₄ cycloalkyl, hydroxy, C₁₋₄alkoxy, cyano, —NR′R″, —CONR′R″, and —CO₂R′; wherein said C₁₋₄alkyl may optionally be substituted one or more times with a substituent independently selected from halogen and C₁₋₄alkoxy; and R′ and R″ are each independently selected from hydrogen and C₁₋₄alkyl.

In some embodiments, R¹¹ and R⁰ together with the atoms attached thereto form a 5-6 membered heterocyclic ring containing one or two N atoms, wherein said 5-6 membered heterocyclic ring containing one or two N atoms is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano,—COF₃, —NR′R″, —CONR′R″ and —CO₂R′, wherein said C₁₋₄alkyl may optionally be substituted one or more times with a substituent independently selected from halogen and C₁₋₄alkoxy.

In some embodiments, R¹¹ and R⁰ together with the atoms attached thereto form a 5-6 membered heteroaromatic ring containing one or two N atoms, wherein said 5-6 membered heteroaromatic ring containing one or two N atoms is optionally substituted one or more times with a substituent independently selected from halogen, C₁₋₄alkyl, halo-C₁₋₄alkyl, C₃₋₄cycloalkyl, hydroxy, C₁₋₄alkoxy, cyano,—COF₃, —NR′R″, —CONR′R″ and —CO₂R′, wherein said C₁₋₄alkyl may optionally be substituted one or more times with a substituent independently selected from halogen and C₁₋₄alkoxy.

In some embodiments, the 5-6 membered heteroaromatic ring containing one or two N atoms is a pyrazole ring or an imidazole ring.

In some embodiments, the 5-6 membered heterocyclic ring containing one or two N atoms is a 1,3-dihydro-2H-imidazol-2-one ring.

In some embodiments, X¹ is CR¹¹, X² is CH, X³ is CH, and X⁴ is CH, and R⁰ and R¹¹ together with the atoms attached thereto form a pyrazole ring, an imidazole ring, or a 1,3-dihydro-2H-imidazol-2-one ring; where said pyrazole, imidazole, or 1.3-dihydro-2H-imidazol-2-one ring may optionally be substituted by a substituent independently selected from halogen, C₁₋₄alkyl, halo-C₁₋₄alkyl, C₃₋₄ cycloalkyl, hydroxy, C₁₋₄alkoxy, cyano, —NR′R″, —CONR′R″, and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl.

In some embodiments, X¹ is CR¹¹, X² is CH, X³ is CH, and X⁴ is CH, and R⁰ and R¹¹ together with the atoms attached thereto form a pyrazole ring or imidazole ring; where said pyrazole or imidazole ring may optionally be substituted by a substituent independently selected from halogen, C₁₋₄alkyl, halo-C₁₋₄alkyl, C₃₋₄ cycloalkyl, hydroxy, C₁₋₄alkoxy, cyano, —NR′R″, —CONR′R″, and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl.

In some embodiments, the pyrazole, imidazole, or a 1,3-dihydro-2H-imidazol-2-one ring is either unsubstituted or substituted with C₁₋₄alkyl or C₁₋₄alkoxy-C₁₋₄alkyl.

In some embodiments, the pyrazole or imidazole ring is either unsubstituted or substituted with C₁₋₄alkyl or C₁₋₄alkoxy-C₁₋₄alkyl.

In some embodiments, R is NHR⁰ and R⁰ is hydrogen or C₁₋₄alkyl (e.g., methyl).

In some embodiments, R is NHR⁰ and R⁰ is C₁₋₄alkyl (e.g., methyl).

In some embodiments, R is NHR⁰ and R⁰ is hydrogen.

In some embodiments, R is NHR⁰ and R⁰ is selected from C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—R′, —(CH₂)ₙ—CO—C₁₋₄alkyl, and —(CH₂)ₙ—CO₂R′; wherein said phenyl is optionally substituted one or more times with substituents independently selected from halogen, C₁₋₄alkyl, halo-C₁₋₄alkyl, C₃₋₄ cycloalkyl, hydroxy, C₁₋₄alkoxy, cyano, —NR′R″, —CONR′R″, and —CO₂R′; wherein R′ and R″ are each independently selected from hydrogen and C₁₋₄alkyl, and n is 0, 1 or 2.

In some embodiments, the

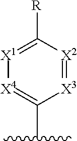

moiety is

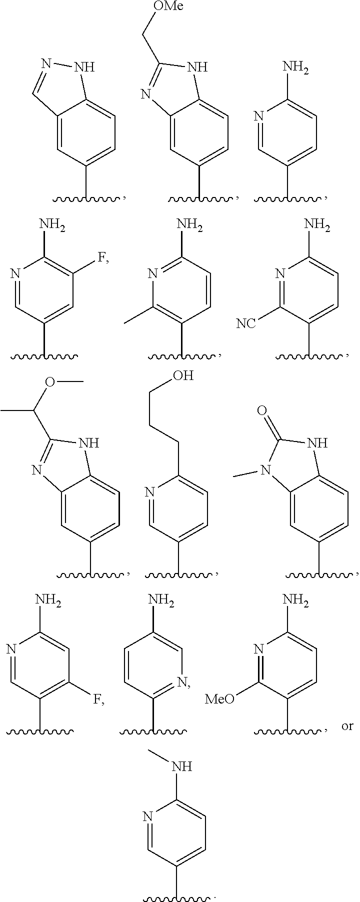

In some embodiments, R^{1a} is hydrogen.

In some embodiments, R¹ is hydrogen.

In some embodiments, R² is C₁₋₅alkyl.

In some embodiments, R² is methyl.

In some embodiments, R³ is hydrogen or C₁₋₄alkyl.

In some embodiments, R³ is hydrogen or methyl.

In some embodiments, R³ is hydrogen.

In some embodiments, R³ is C₁₋₄alkyl.

In some embodiments, R³ is methyl.

In some embodiments, R^{1a} is hydrogen, R¹ is hydrogen, R² is methyl, and R³ is hydrogen or methyl.

In some embodiments, R^{1a} and R¹ are both hydrogen, and R² and R³ are both methyl.

In some embodiments, Z is CR¹⁰ and R¹⁰ is hydrogen.

In some embodiments, X⁵ is CR⁴R⁴.

In some embodiments, X⁵ is CR⁴R⁴ and both R⁴ are hydrogen.

In some embodiments, X⁵ is N.

In some embodiments, X⁵ is N or CR⁴R⁴ and both R⁴ are hydrogen.

In some embodiments, R⁵ is hydrogen and R^{5a} is hydrogen.

In some embodiments, Z is CR¹⁰, and R⁵ and R¹⁰ together form a bond between the two carbons to which they are attached.

In some embodiments, the

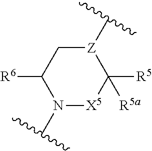

moiety is

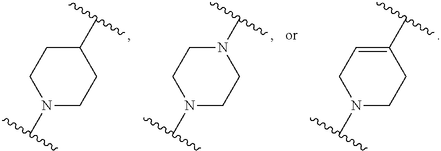 [SMILES: *C1CCN(*)CC1 ; *N1CCN(*)CC1 ; *C1=CCN(*)CC1]

In some embodiments, Y¹ is CR⁷ and Y² is N or CR⁸.

In some embodiments, Y¹ is CR⁷ and Y² is CR⁸.

In some embodiments, R⁷ and R⁸ are independently selected from hydrogen, halogen and C₁₋₄alkyl.

In some embodiments, R⁷ and R⁸ are independently selected from halogen (e.g., fluoro) and C₁₋₄alkyl (e.g., methyl).

In some embodiments, Y¹ is CR⁷; Y² is N or CR⁸; and R⁷ and R⁸ are independently selected from halogen (e.g., fluoro) and C₁₋₄alkyl (e.g., methyl).

In some embodiments, both of R⁷ and R⁸ are hydrogen.

In some embodiments, the

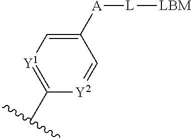

moiety is

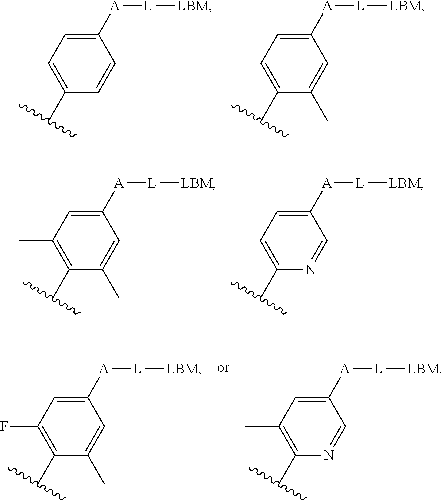

In some embodiments, provided is a compound of formula (I-b), (II-b), or (III-b):

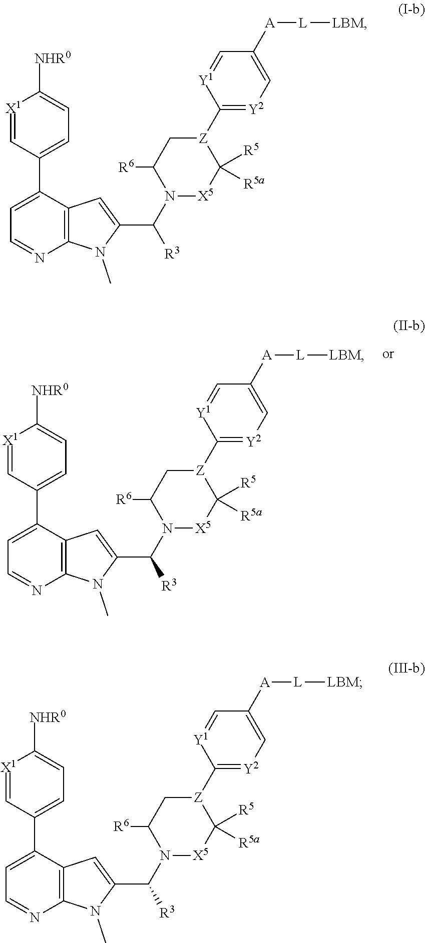

wherein R³ is C₁₋₄alkyl;
or a pharmaceutically acceptable salt or stereoisomer thereof.

### Precursor

In some embodiments, compounds of formula (I) are prepared from precursors in the following table, and subsequently derivatized at the appropriate position to achieve compounds of formula (I) (i.e, wherein R⁹ is -A-L-LBM).

| Ex- | |
|---|---|
| am- | |
| ple | Structure |
| 1aaa2 | 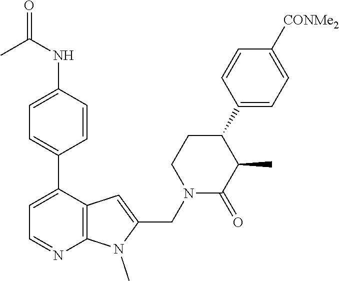 |
| 1aa2′ | 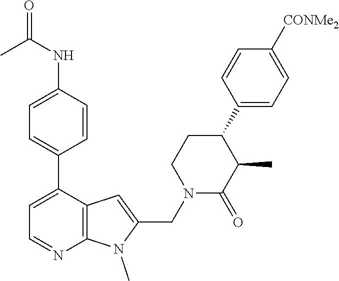 |
| 1ad1 | 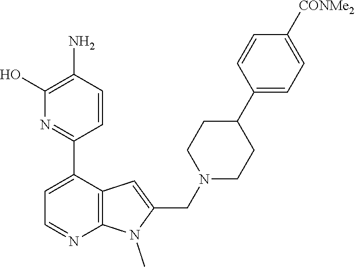 |
| 1ad2 | 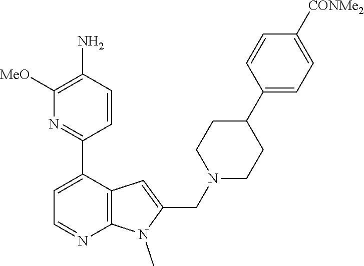 |
| 1ad3 | 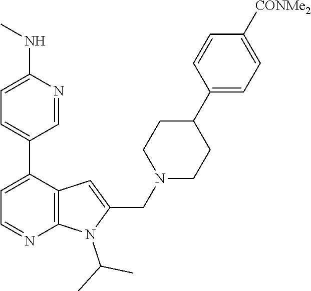 |
| 1ae4 | 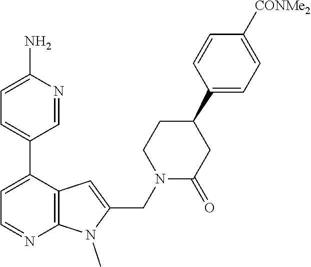 |
| 1ae5 | 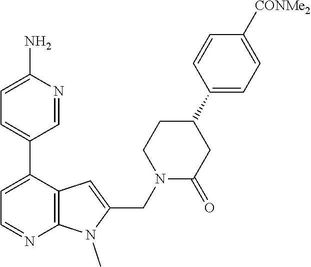 |
| 1ae7 | 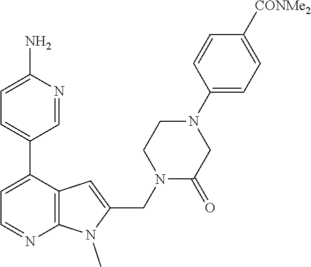 |
| 1ae10 | 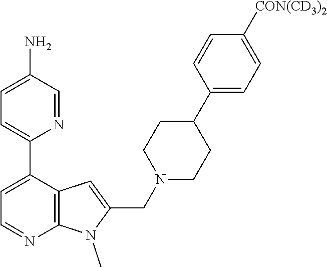 |
| 1ae14 | 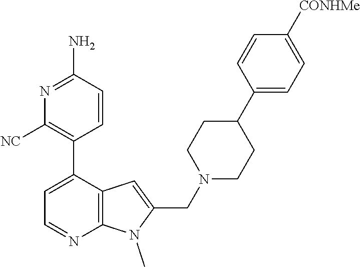 |
| 1ae15 | 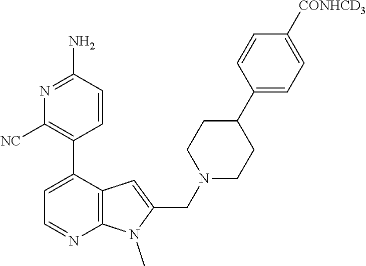 |
| 1ae19 | 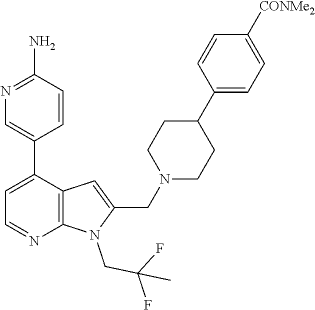 |
| 1ae22 | 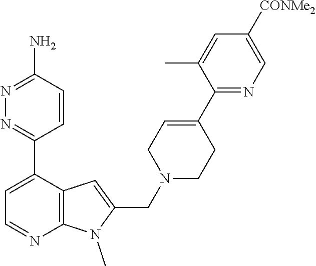 |
| 1af7 | 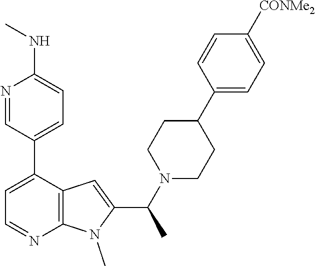 |
| 1af8 | 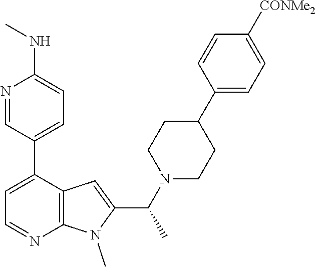 |
| 1af11 | 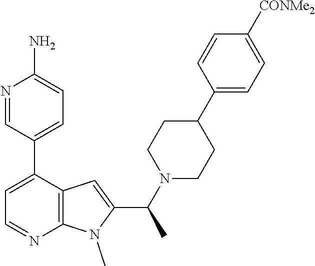 |
| 1af12 | 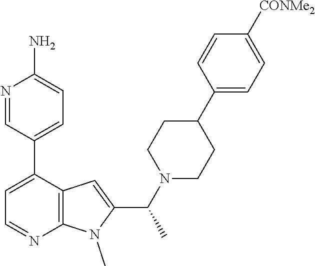 |
| 1af13 | 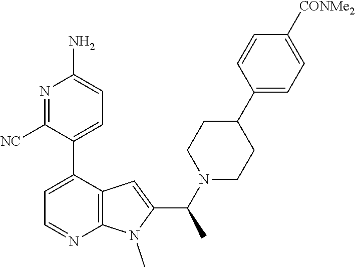 |
| 1af14 | 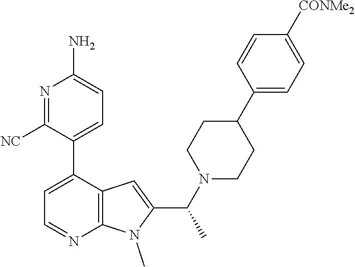 |
| 1af15 | 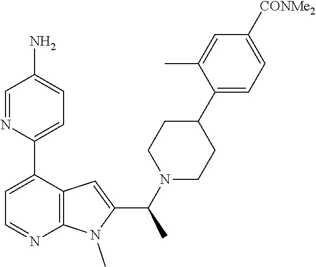 |
| 1af16 | 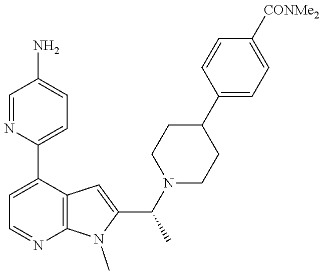 |
| 1af17 | 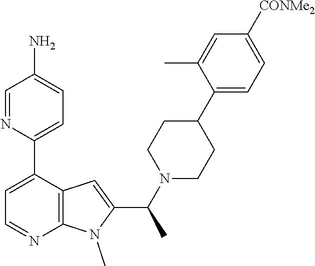 |
| 1af18 | 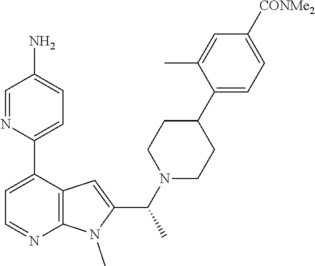 |
| 1af19 | 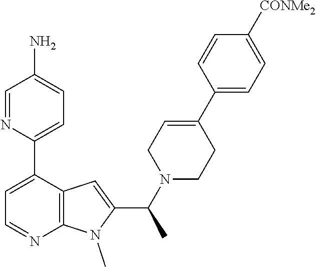 |
| 1af20 | 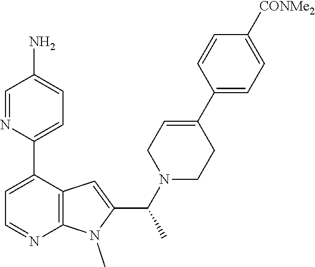 |
| 1af21 | 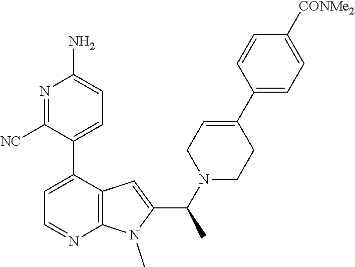 |
| 1af23 | 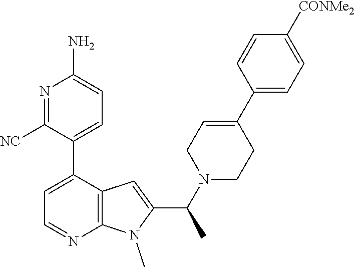 |
| 1af22 | 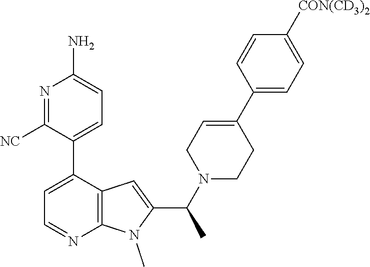 |
| 1af24 | 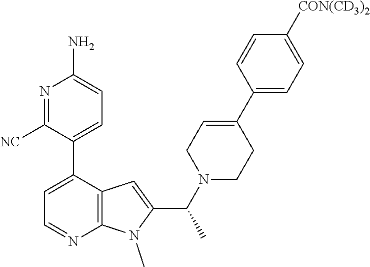 |
| 1af25 | 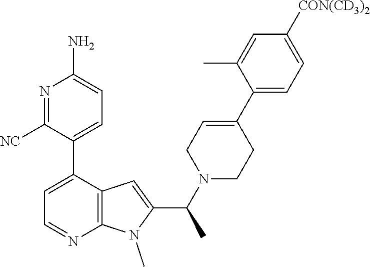 |
| 1af26 | 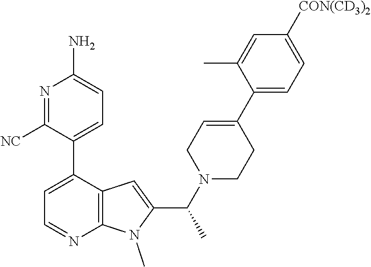 |
| 1af27 | 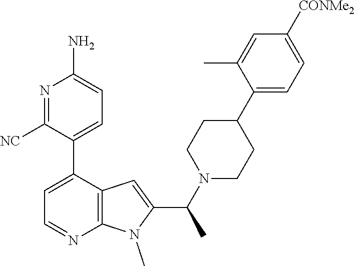 |
| 1af28 | 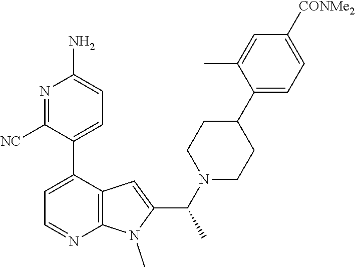 |
| 1af29 | 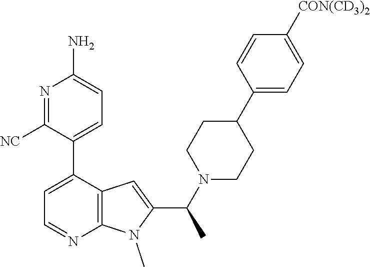 |
| 1af30 | 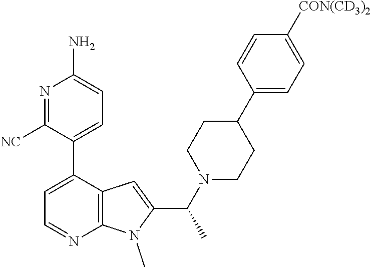 |
| 1af31 | 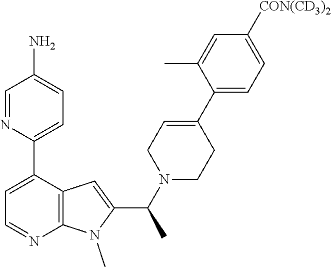 |
| 1af33 | 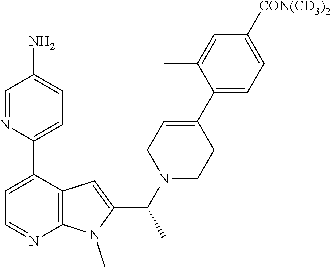 |
| 1af32 | 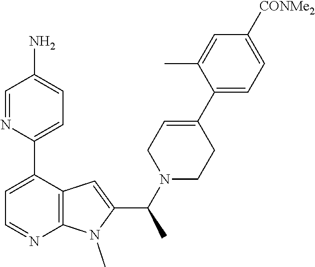 |
| 1af34 | 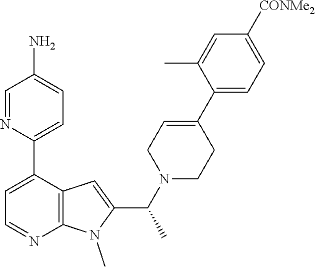 |
| 1af35 | 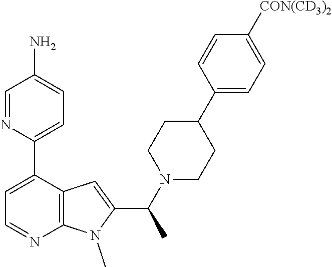 |
| 1af36 | 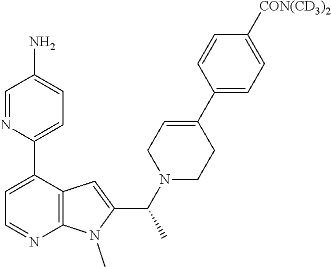 |
| 1af37 | 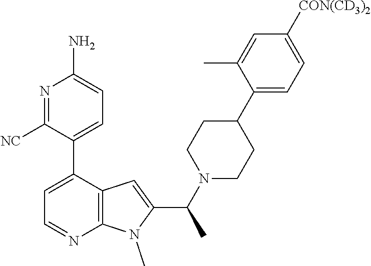 |
| 1af38 | 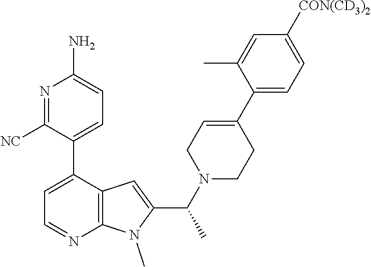 |
| 1af39 | 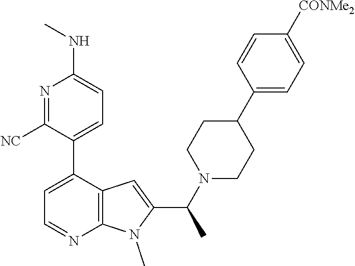 |
| 1af40 | 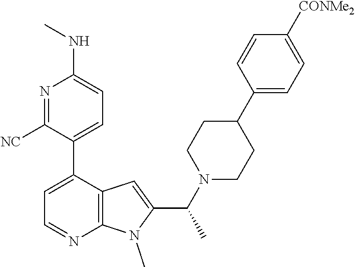 |
| 1af41 | 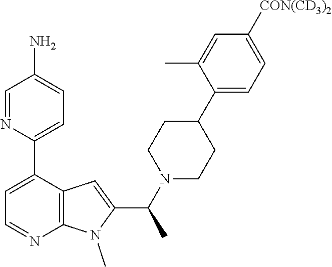 |
| 1af42 | 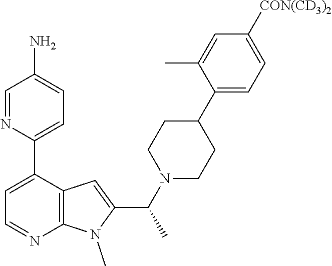 |
| 1af43 | 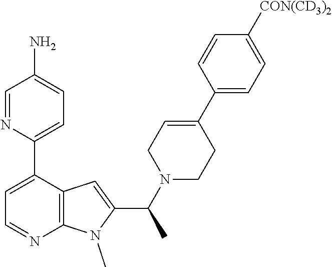 |
| 1af46 | 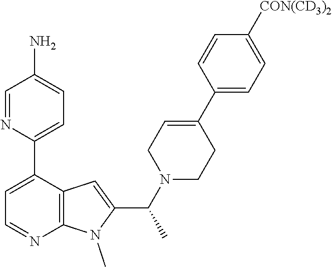 |
| 1af44 | 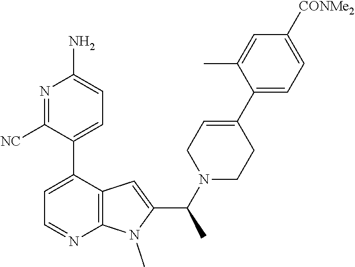 |
| 1af45 | 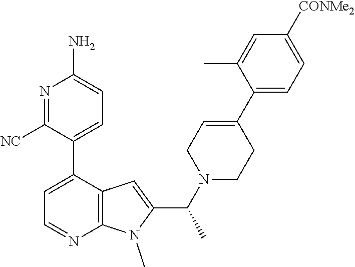 |
| 1af50 | 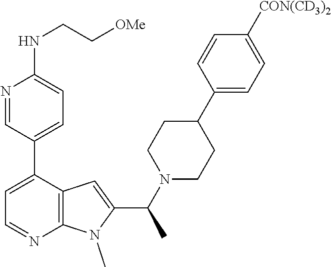 |
| 1af51 | 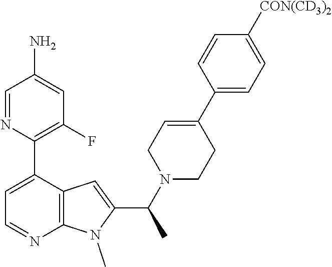 |
| 1af52 | 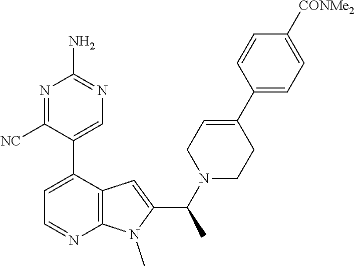 |
| 1af53 | 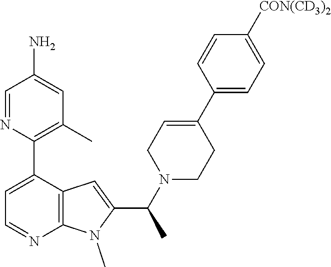 |
| 1af54 | 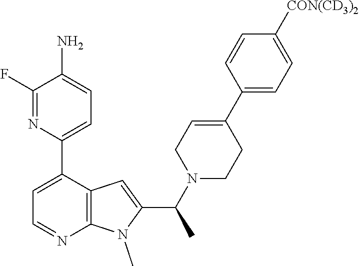 |
| 1af55 | 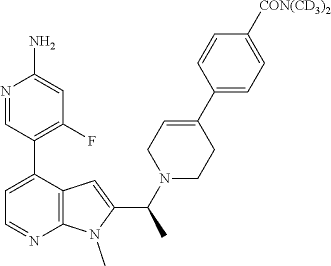 |
| 1af56 | 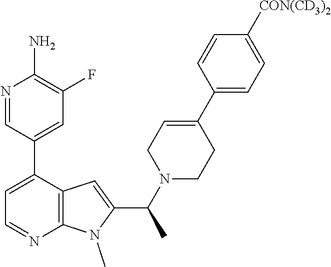 |
| 1af57 | 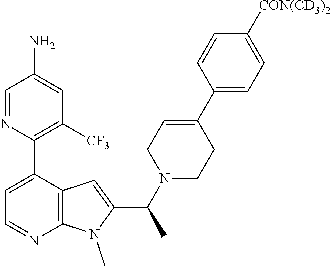 |
| 1af58 | 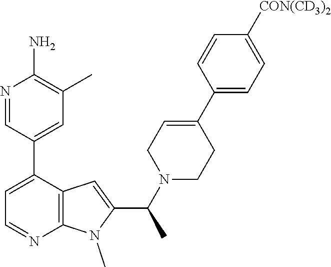 |
| 1af59 | 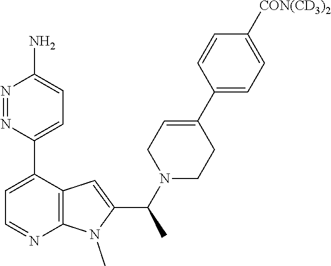 |
| 1af60 | 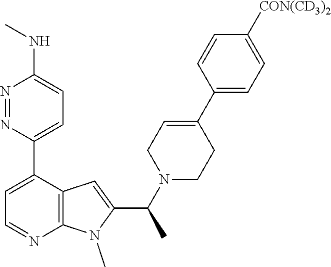 |
| 1af64 | 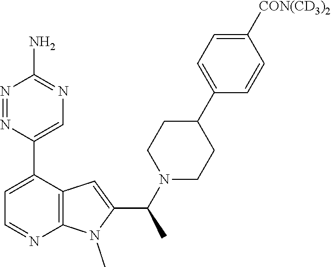 |
| 1af66 | 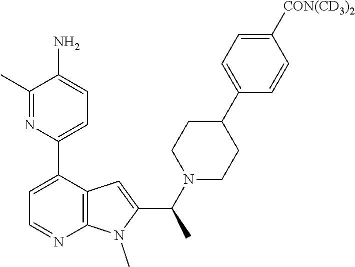 |
| 1af67 | 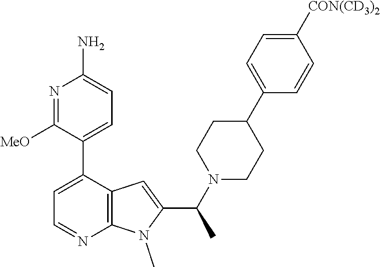 |
| 1af68 | 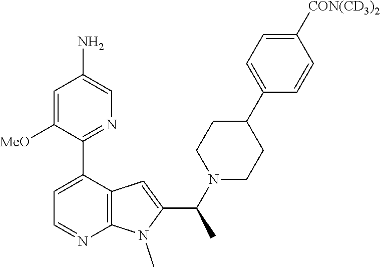 |
| 1af69 | 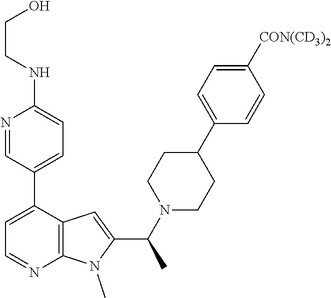 |
| 1af70 | 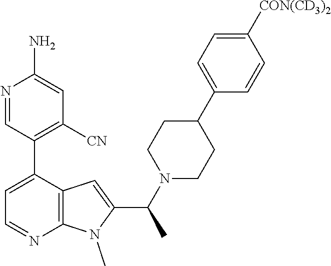 |
| 1af72 | 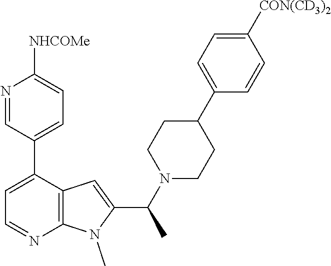 |
| 1af73 | 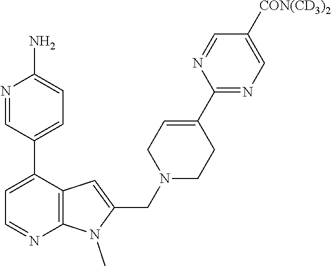 |
| 1af75 | 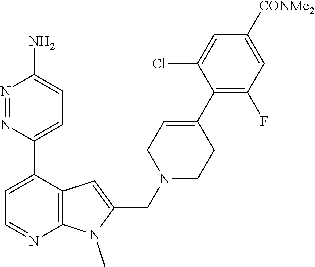 |
| 1af76 | 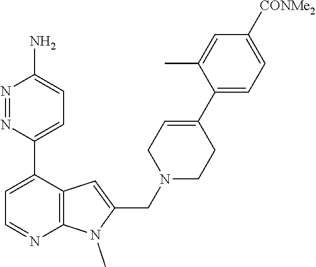 |
| 1af77 | 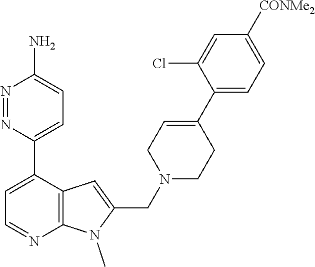 |
| 1af78 | 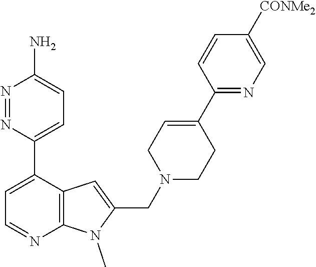 |
| 1af79 | 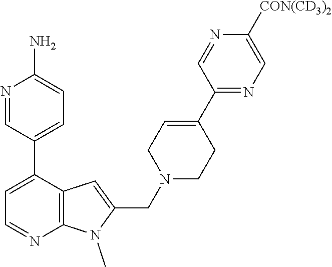 |
| 1af80 | 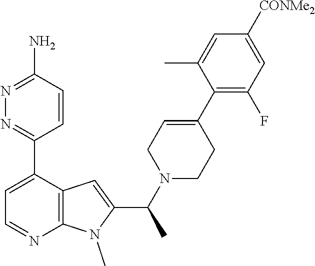 |
| 1af81 | 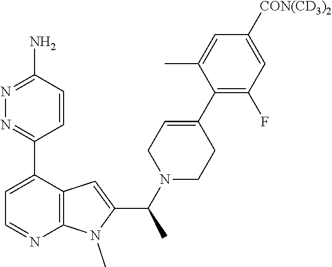 |
| 1j6 | 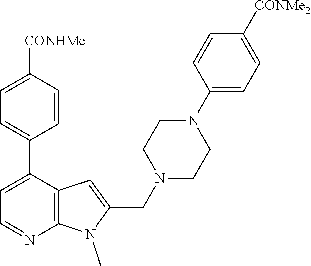 |
| 1j7 | 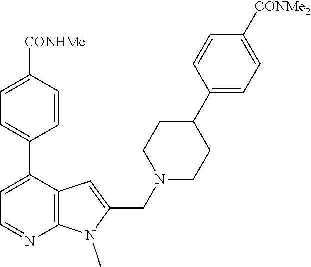 |
| 1j13 | 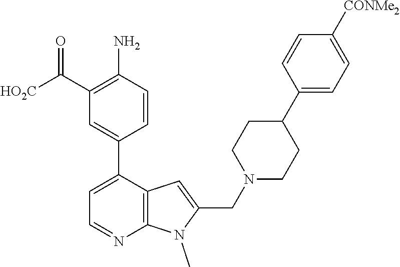 |
| 1k2 | 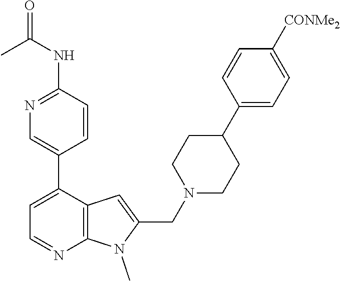 |
| 1k3 | 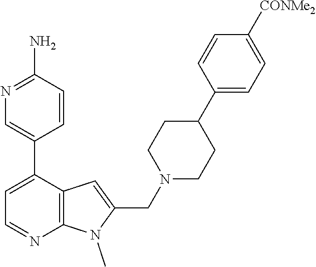 |
| 1k6 | 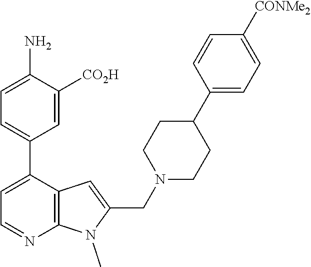 |
| 1k7 | 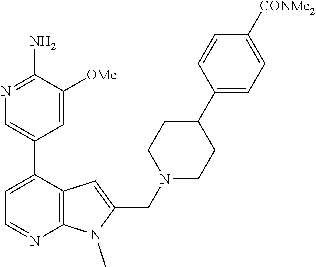 |
| 1k8 | 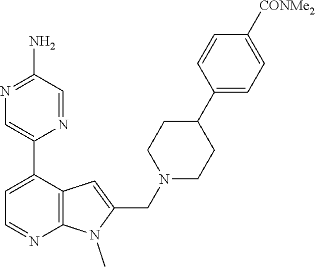 |
| 1k9 | 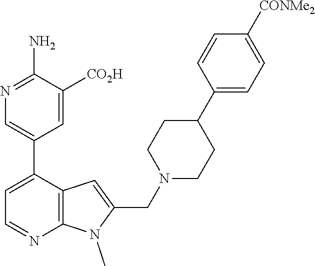 |
| 1k10 | 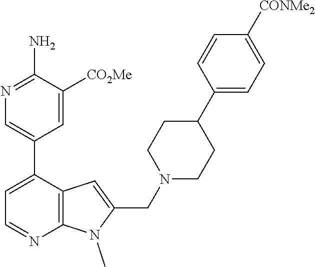 |
| 1k11 | 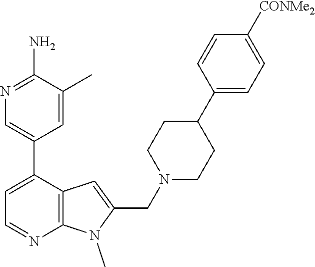 |
| 1k12 | 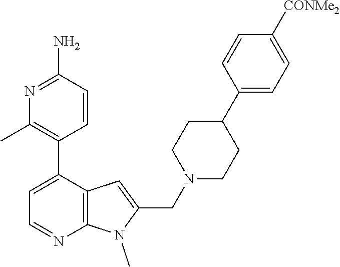 |
| 1k13 | 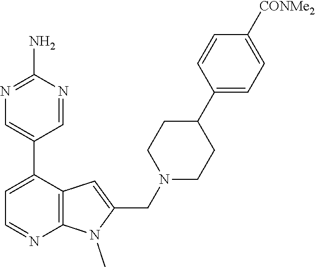 |
| 1k14 | 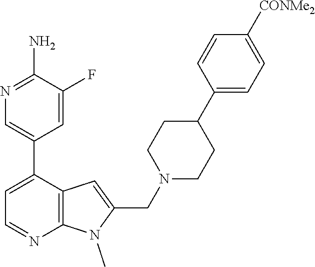 |
| 1k15 | 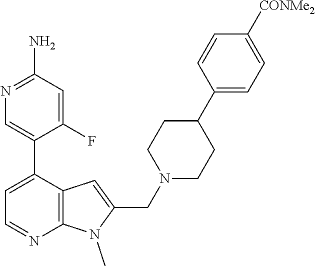 |
| 1k16 | 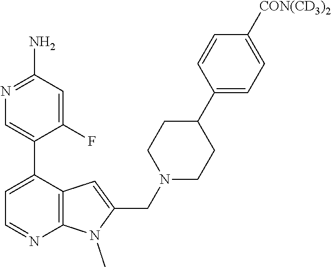 |
| 1k17 | 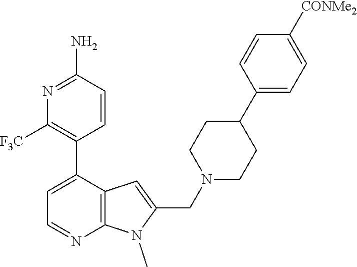 |
| 1k18 | 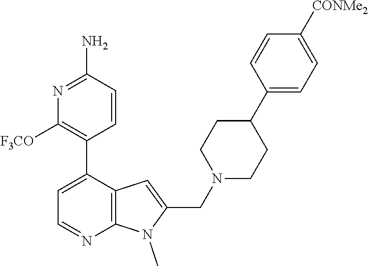 |
| 1k19 | 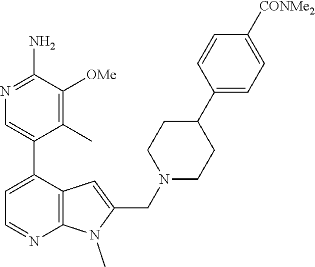 |
| 1k20 | 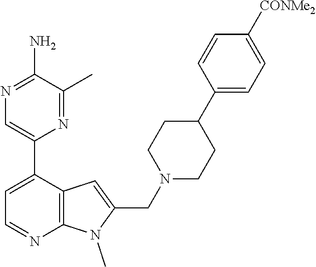 |
| 1k21 | 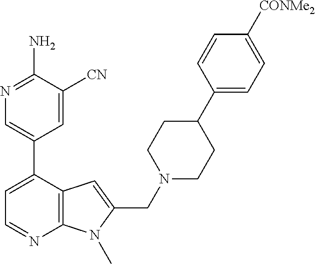 |
| 1k22 | 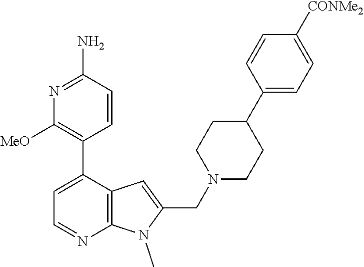 |
| 1k23 | 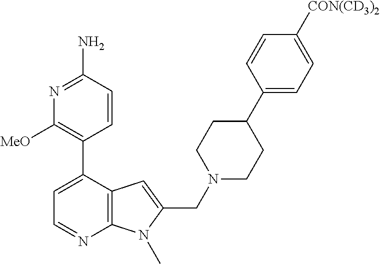 |
| 1k24 | 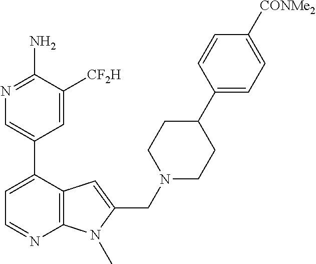 |
| 1k25 | 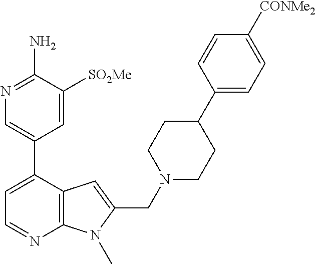 |
| 1k26 | 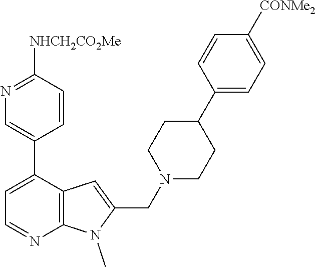 |
| 1k27 | 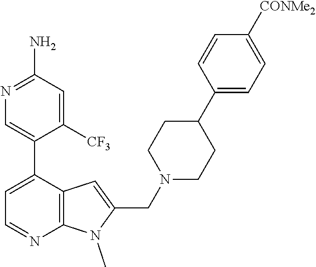 |
| 1k28 | 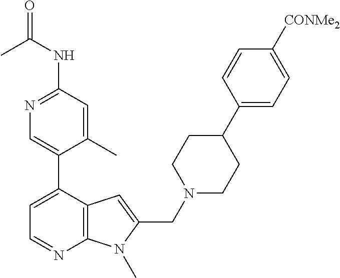 |
| 1k30 | 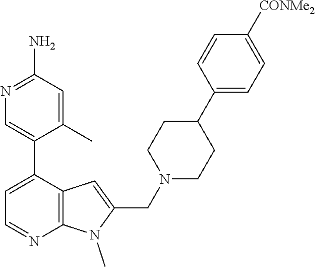 |
| 1k31 | 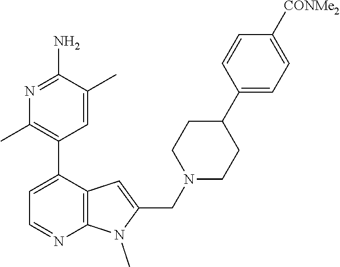 |
| 1k32 | 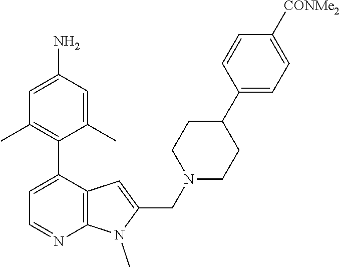 |
| 1k33 | 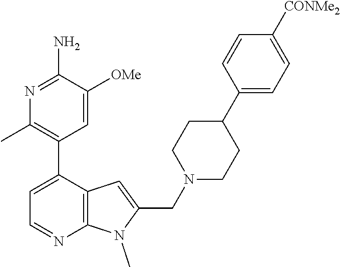 |
| 1k34 | 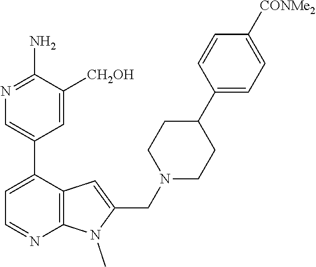 |
| 1k35 | 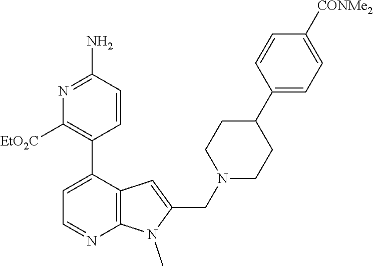 |
| 1k36 | 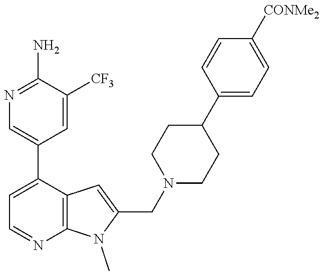 |
| 1k37 | 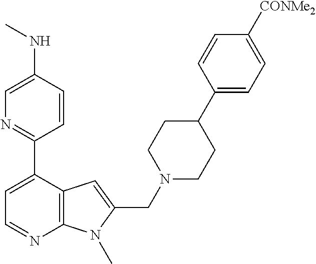 |
| 1k38 | 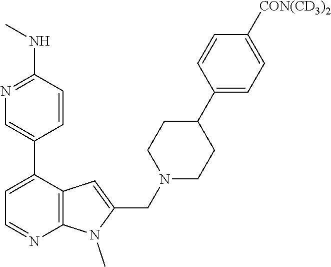 |
| 1k39 | 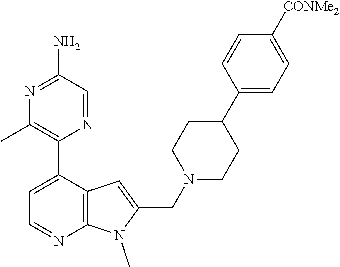 |
| 1k40 | 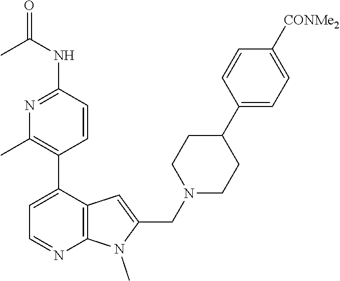 |
| 1k41 | 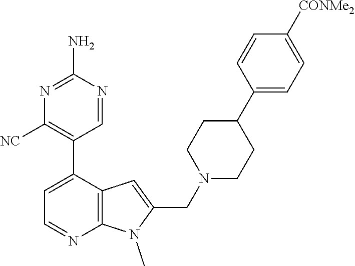 |
| 1k42 | 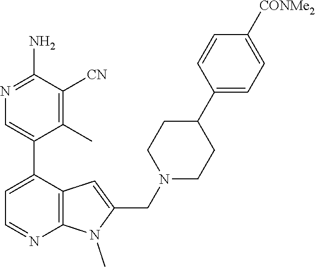 |
| 1k43 | 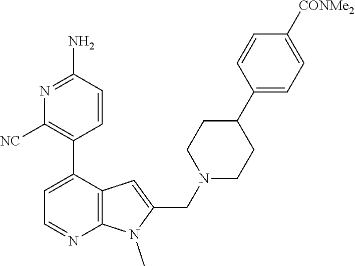 |
| 1k44 | 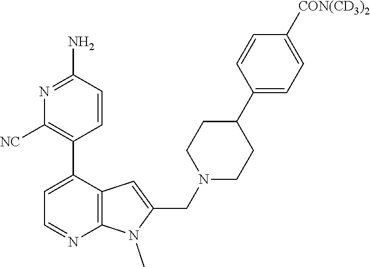 |
| 1k45 | 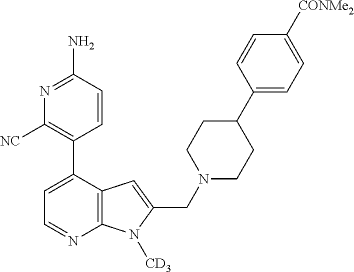 |
| 1k47 | 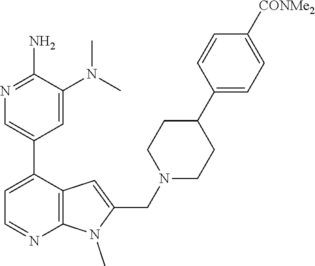 |
| 1k48 | 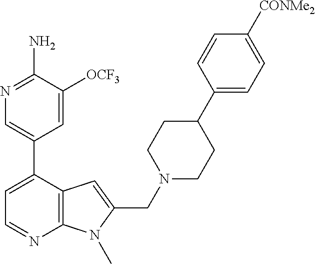 |
| 1k49 | 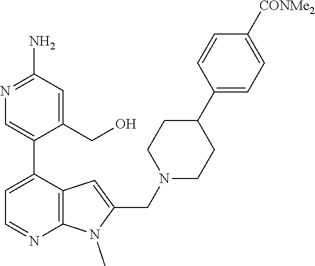 |
| 1k50 | 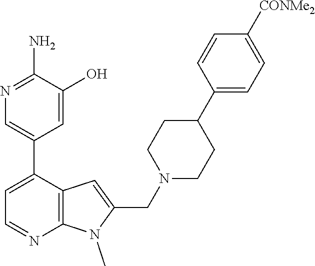 |
| 1k51 | 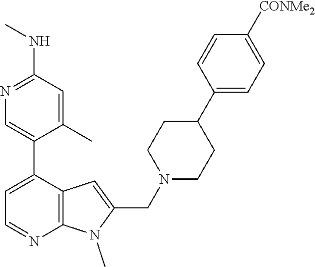 |
| 1k52 | 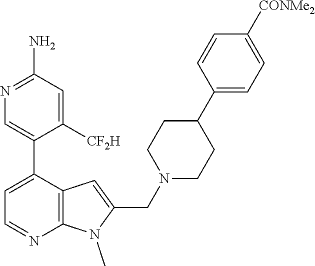 |
| 1k53 | 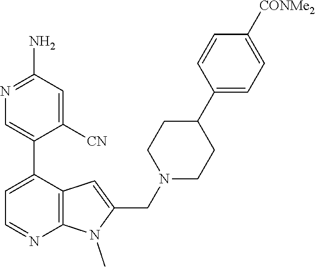 |
| 1k54 | 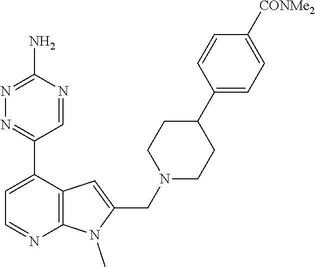 |
| 1k55 | 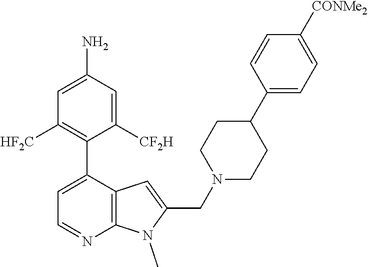 |
| 1k56 | 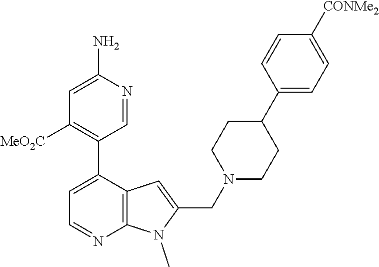 |
| 1k57 | 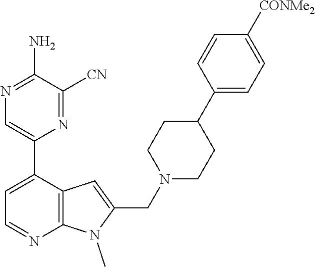 |
| 1k58 | 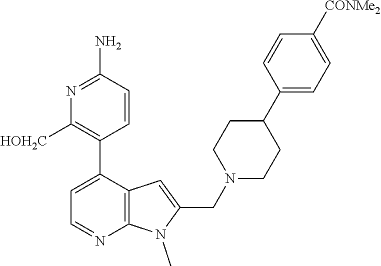 |
| 1k59 | 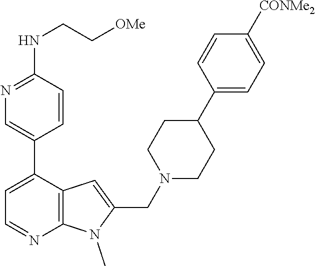 |
| 1k60 | 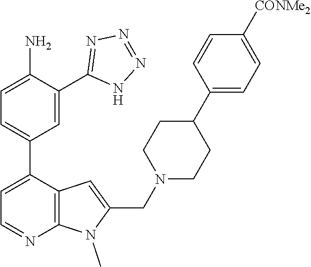 |
| 1k61 | 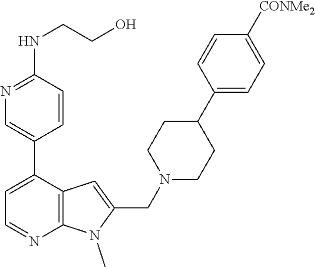 |
| 1k63 | 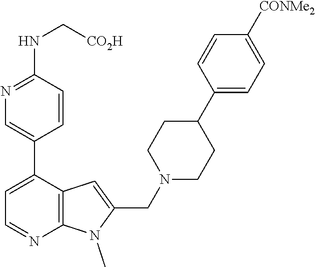 |
| 1k64 | 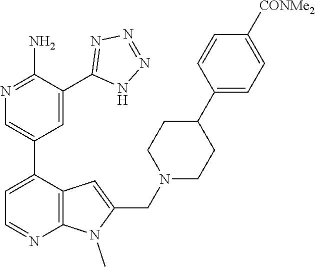 |
| 1k65 | 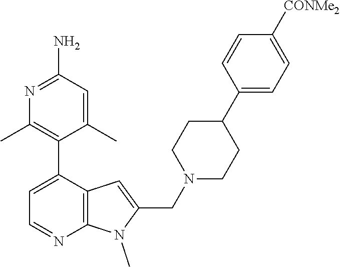 |
| 1k66 | 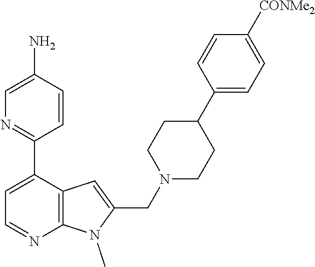 |
| 1k67 | 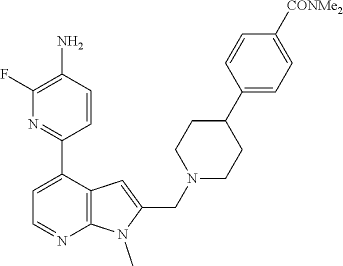 |
| 1k68 | 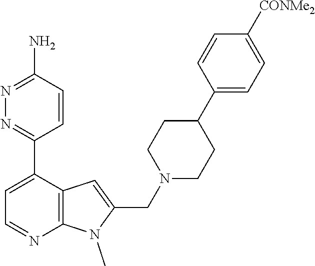 |
| 1k69 | 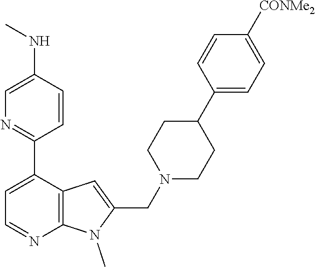 |
| 1k70 | 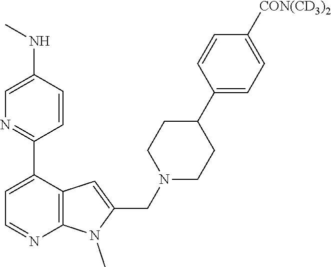 |
| 1k71 | 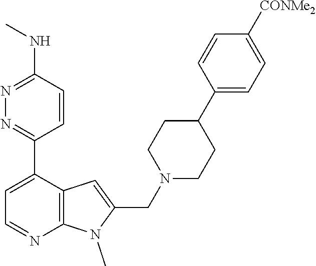 |
| 1k72 | 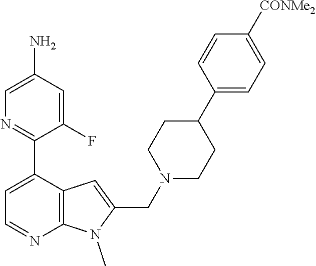 |
| 1k73 | 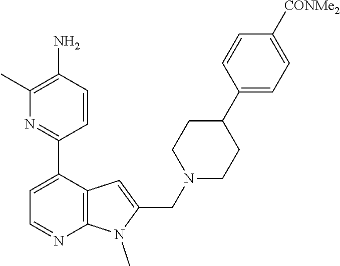 |
| 1k74 | 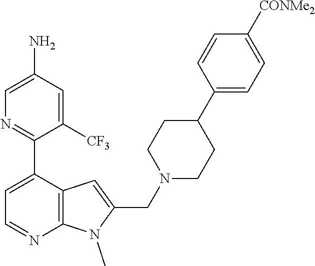 |
| 1k75 | 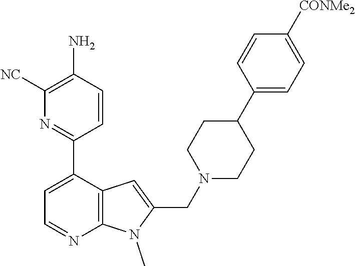 |
| 1k76 | 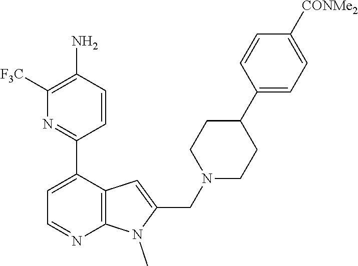 |
| 1k77 | 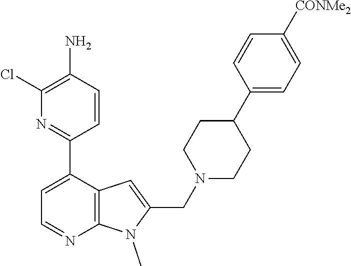 |
| 1k78 | 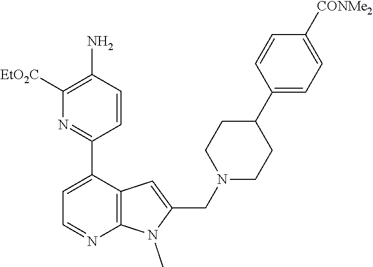 |
| 1k79 | 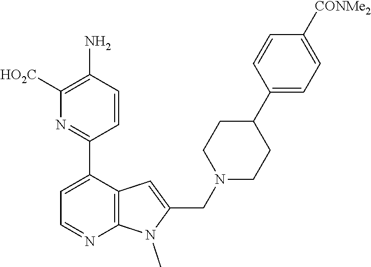 |
| 1k80 | 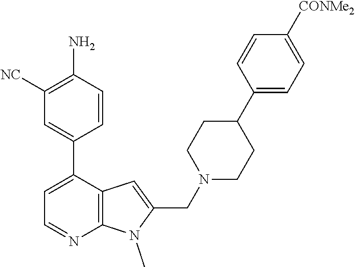 |
| 1m145 | 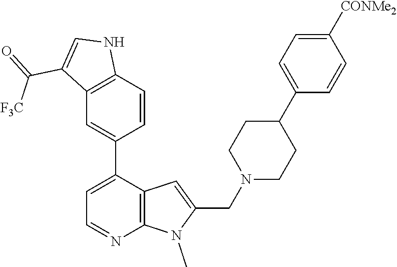 |
| 1n4 | 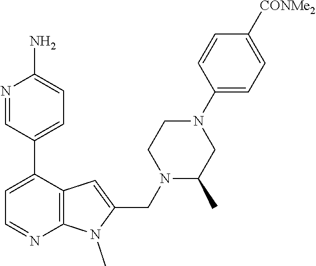 |
| 1n5 | 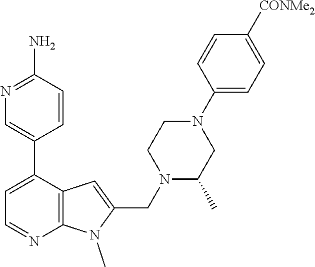 |
| 1n6 | 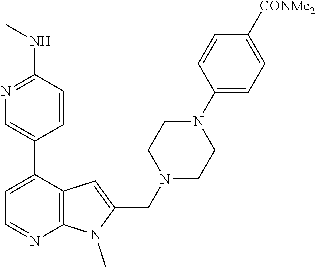 |
| 1n7 | 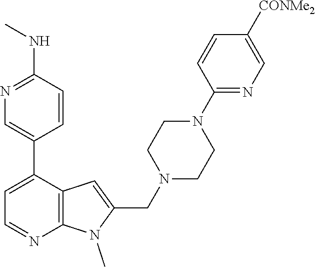 |
| 1n8 | 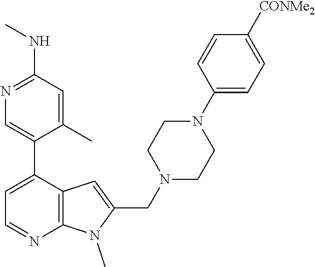 |
| 1n9 | 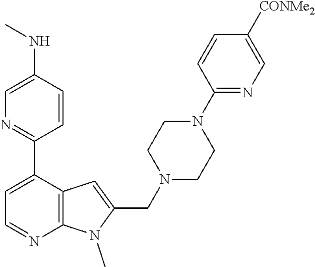 |
| 1o1 | 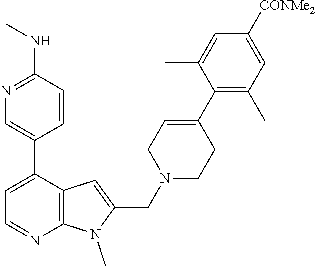 |
| 1o2 | 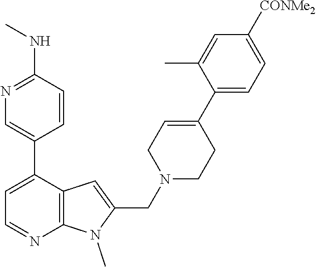 |
| 1o3 | 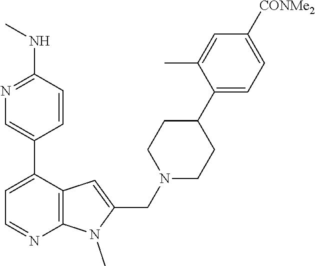 |
| 1o4 | 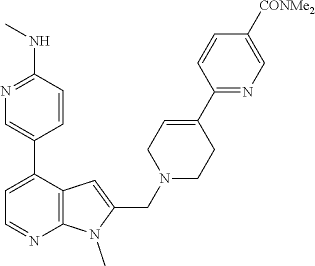 |
| 1o5 | 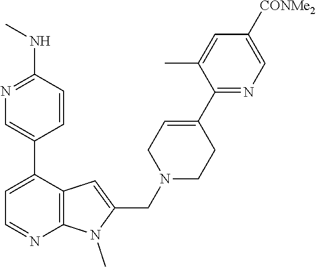 |
| 1o6 | 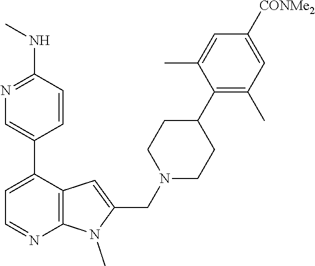 |
| 1o7 | 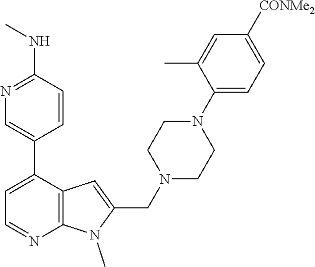 |
| 1o8 | 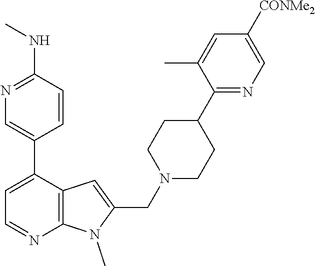 |
| 1o17 1o18 | 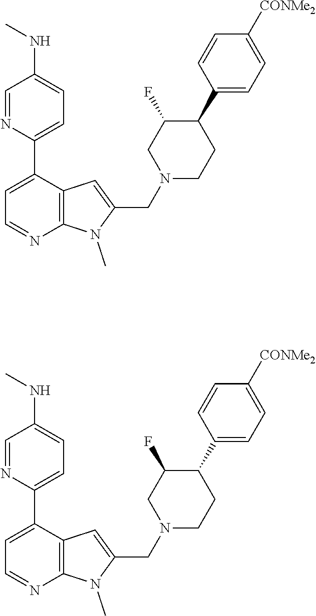 |
| 1o19 | 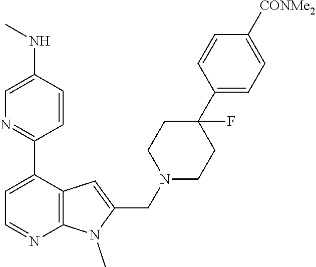 |
| 1o20 | 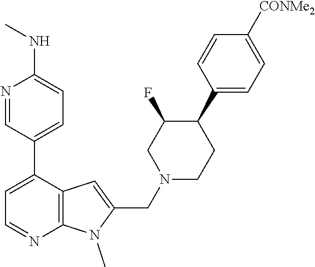 |
| 1o21 | 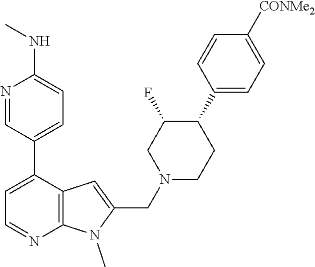 |
| 1o23 | 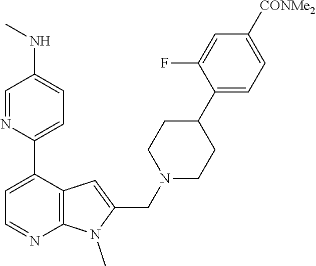 |
| 1o24 | 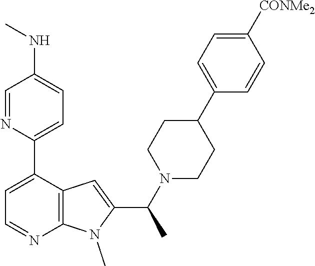 |
| 1o25 | 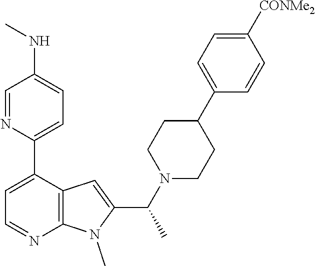 |
| 1026 | 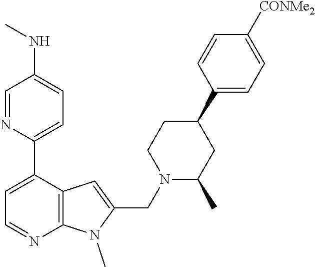 |
| 1o27 | 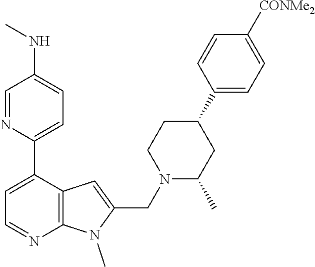 |
| 1o28 | 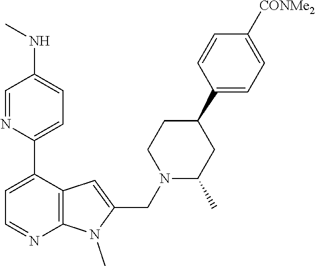 |
| 1o29 | 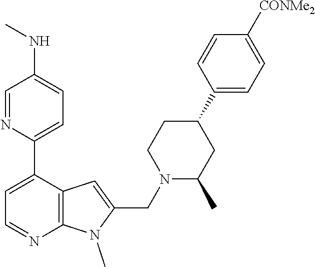 |
| 1p1 | 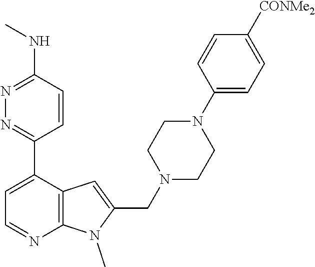 |
| 1p2 | 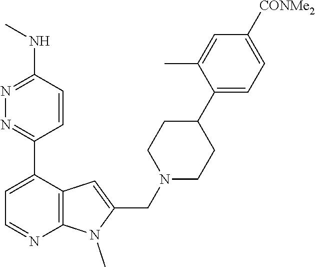 |
| 1p3 | 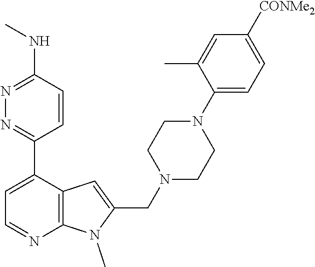 |
| 1p4 | 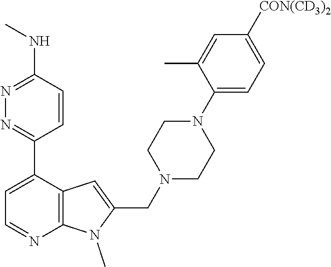 |
| 1p5 | 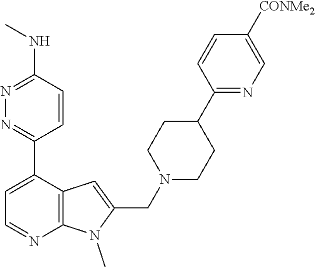 |
| 1p6 | 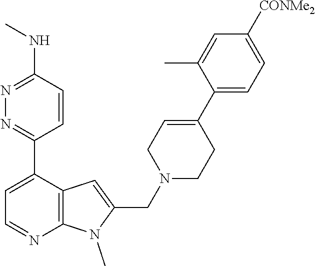 |
| 1p7 | 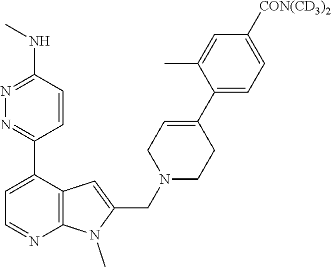 |
| 1p8 | 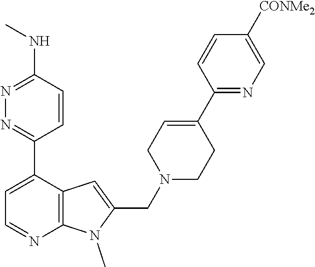 |
| 1p9 | 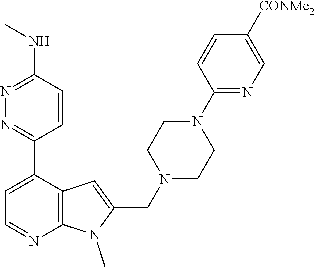 |
| 1p10 | 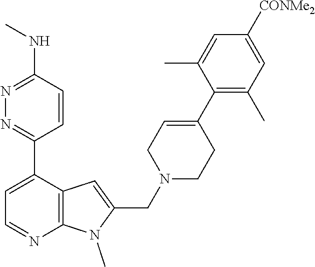 |
| 1p11 | 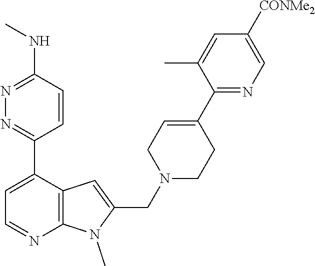 |
| 1p14 | 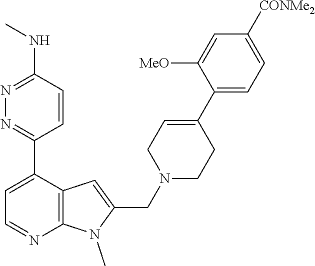 |
| 1p15 | 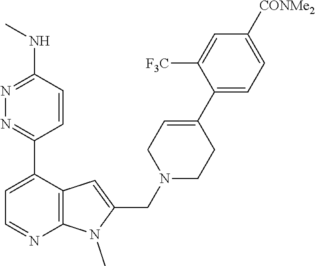 |
| 1p17 | 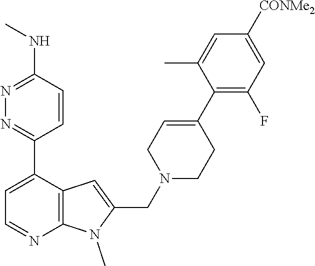 |
| 1p18 | 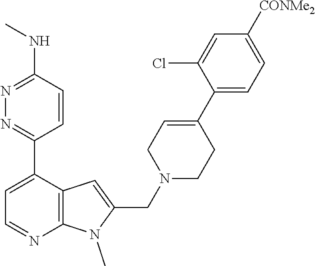 |
| 1p20 | 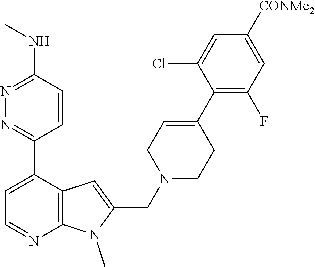 |
| 1q1 | 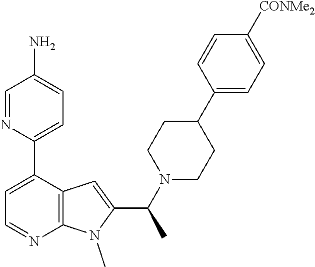 |
| 1q6 | 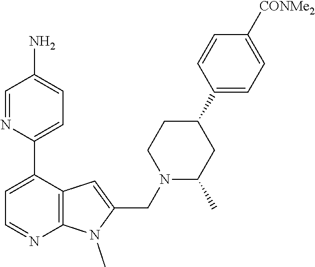 |
| 1q2 | 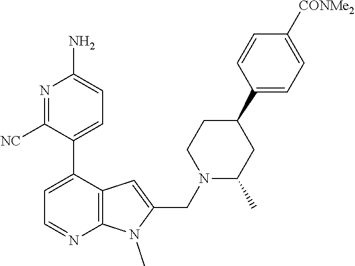 |
| 1q3 | 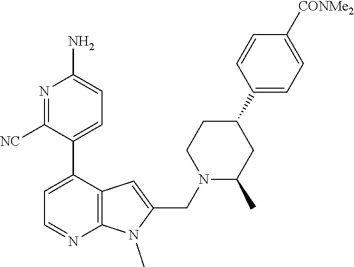 |
| 1q4 | 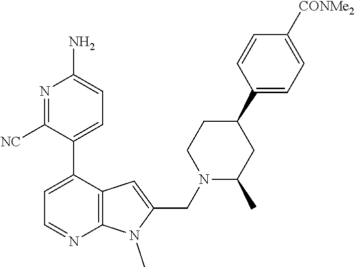 |
| 1q5 | 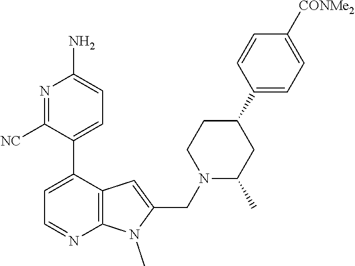 |
| 1q7 | 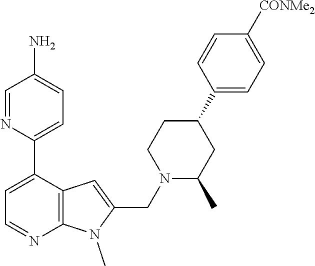 |
| 1q8 | 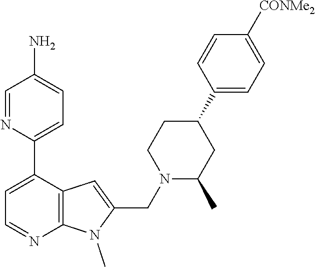 |
| 1r5 | 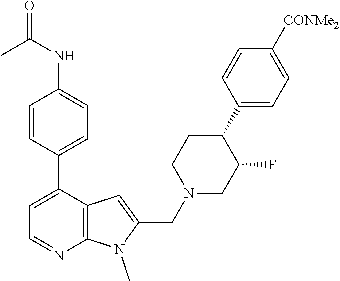 |
| 1r6 | 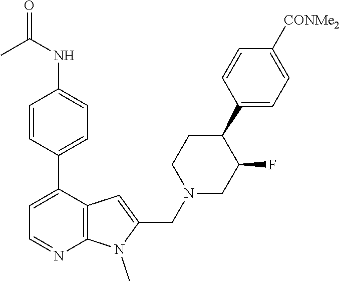 |
| 1s1 | 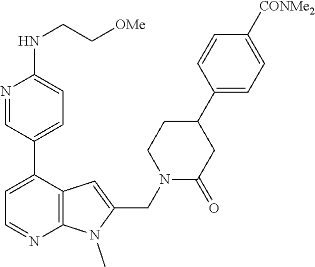 |
| 1s2 | 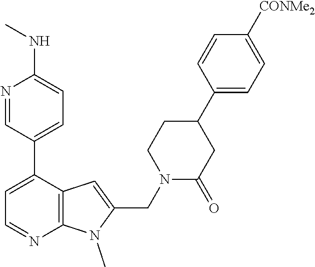 |
| 1s3 | 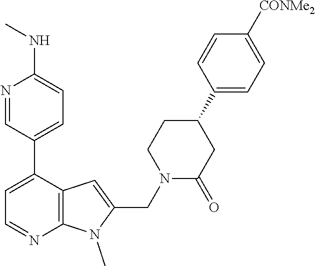 |
| 1s4 | 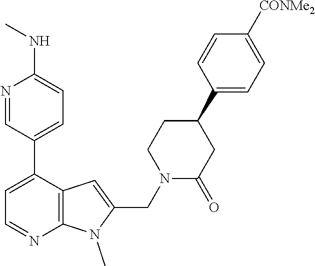 |
| 1s5 | 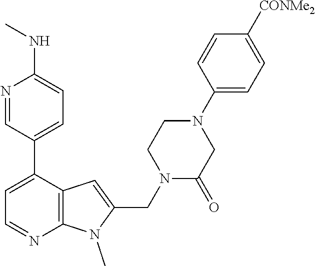 |
| 1s6 | 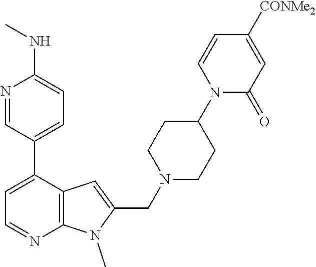 |
| 1s8 | 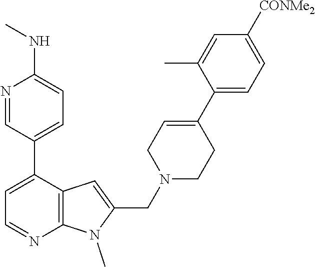 |
| 1s9 | 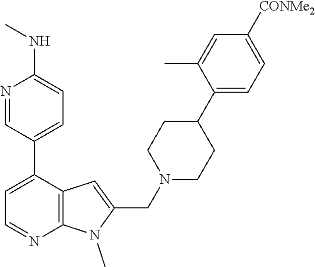 |
| 1s10 | 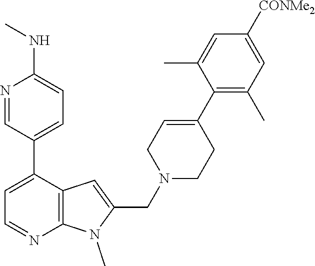 |
| 1s11 | 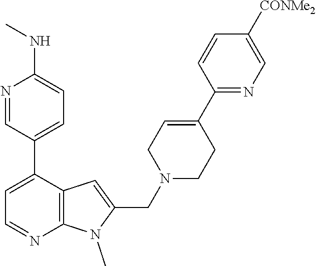 |
| 1s12 | 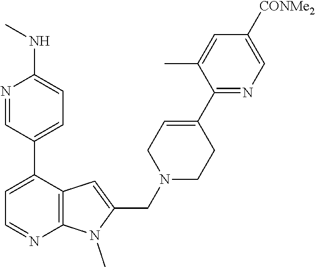 |
| 1s13 | 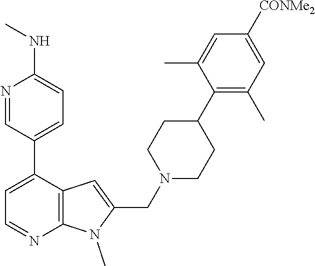 |
| 1s14 | 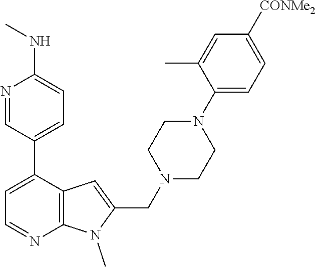 |
| 1s15 | 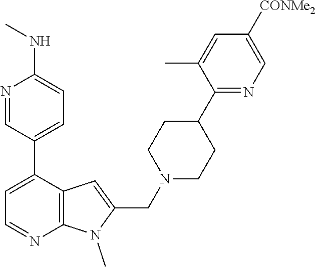 |
| 1s24 | 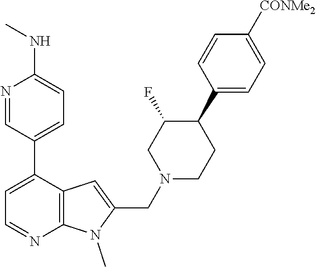 |
| 1s25 | 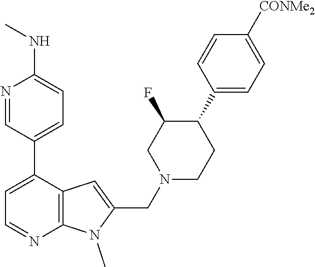 |
| 1s26 | 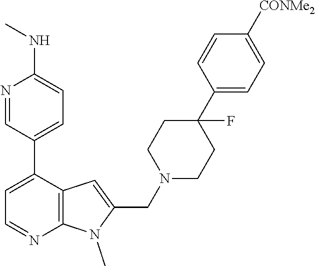 |
| 1s27 | 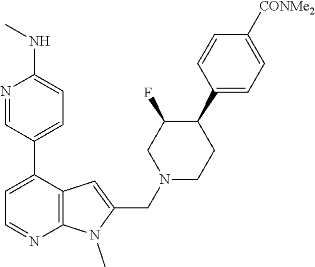 |
| 1s28 | 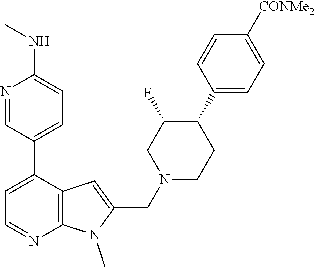 |
| 1s30 | 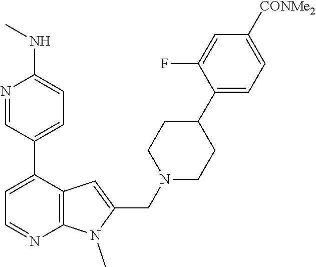 |
| 1u2 | 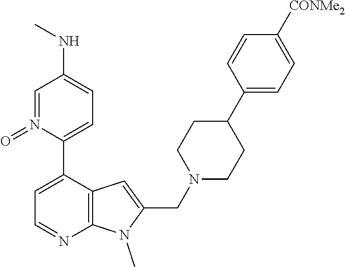 |
| | OR |
| | 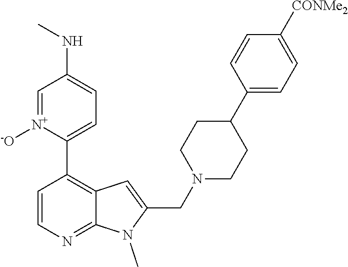 |
| 1u3 | 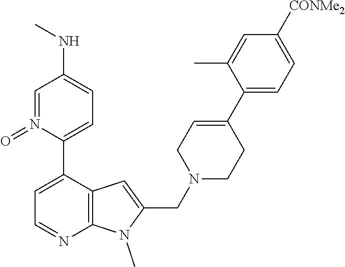 |
| | OR |
| | 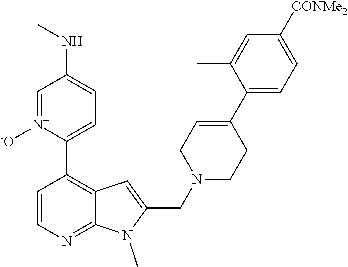 |
| 1u5 | 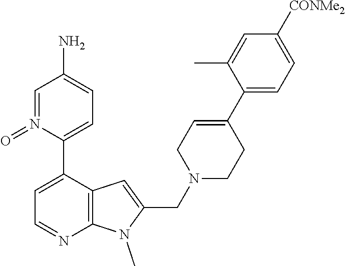 |
| | OR |
| | 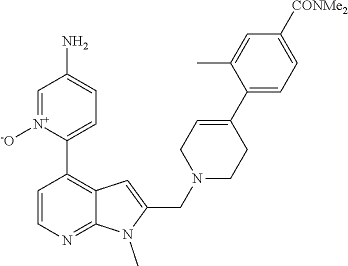 |
| 1x1 | 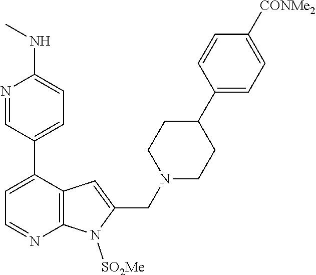 |
| 1x2 | 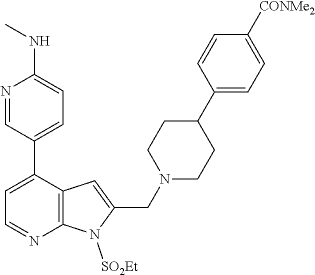 |
| 1x3 | 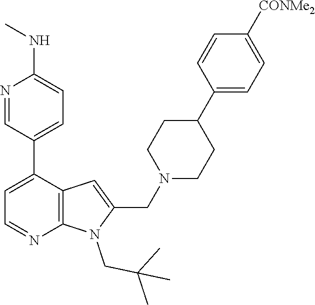 |
| 1y1 | 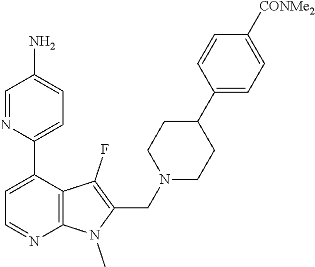 |
| 1z1 | 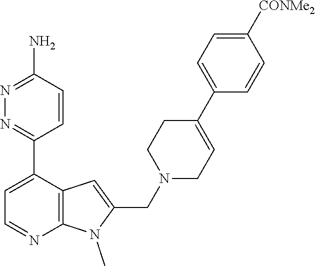 |
| 1z2 | 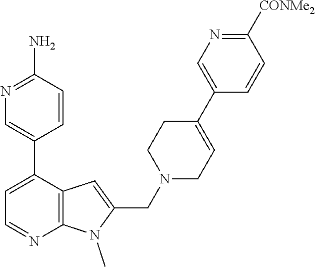 |
| 1z3 | 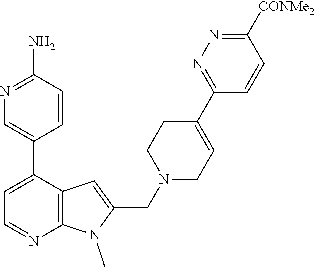 |
| 1z4 | 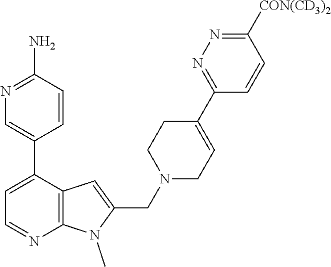 |
| 1z5 | 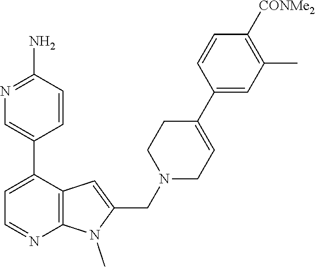 |
| 1z6 | 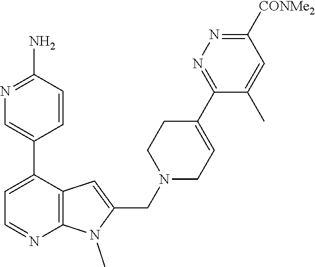 |
| 1z7 | 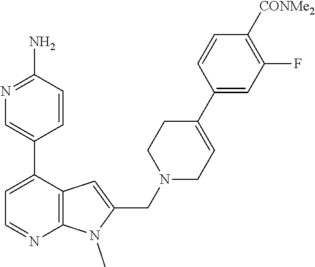 |
| 1z9 | 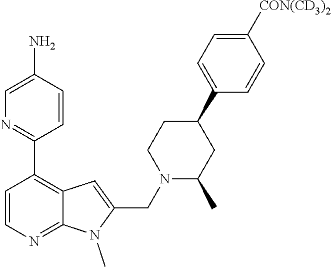 |
| 1z10 | 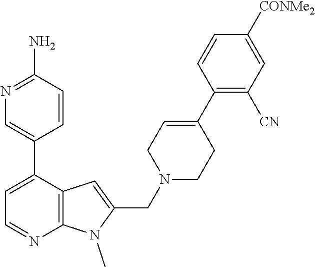 |
| 1z11 | 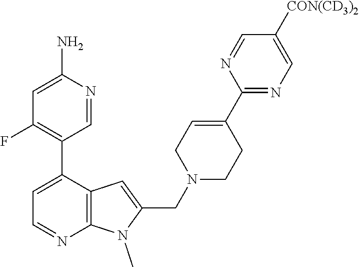 |
| 1z12 | 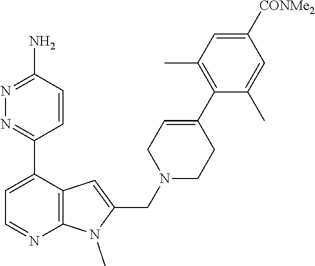 |
| 1z13 | 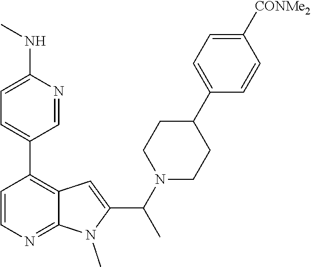 |
| 1z14 | 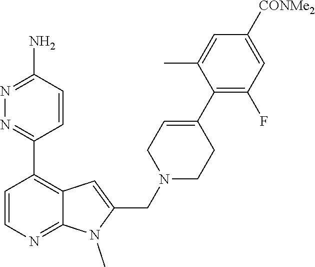 |
| 1z15 | 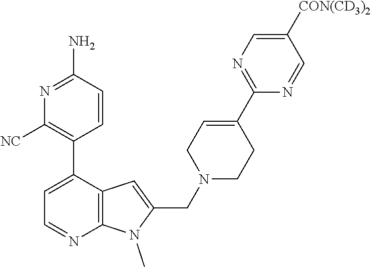 |
| 1z16 | 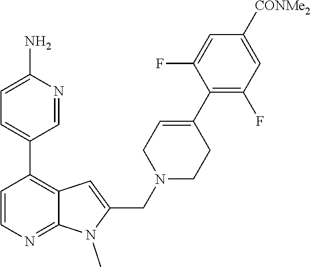 |
| 1z17 | 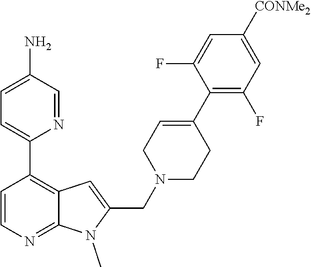 |
| 1z18 | 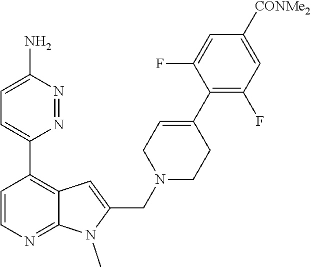 |
| 1z19 | 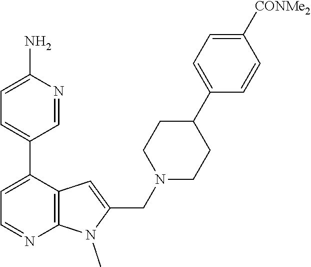 |
| 1z20 | 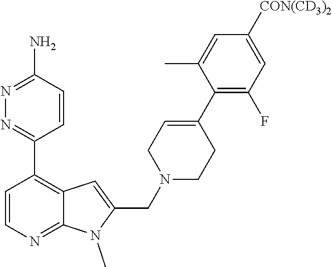 |
| 1z21 | 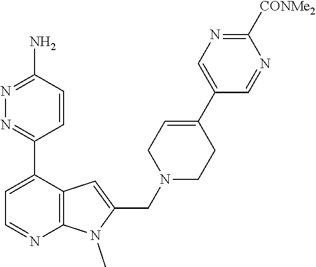 |
| 1z22 | 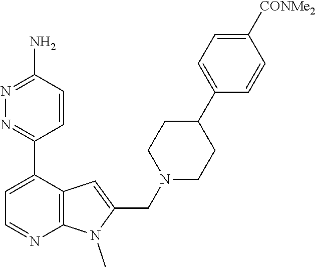 |
| 1z23 | 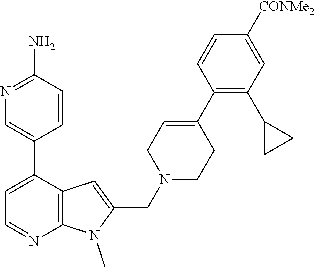 |
| 1z24 | 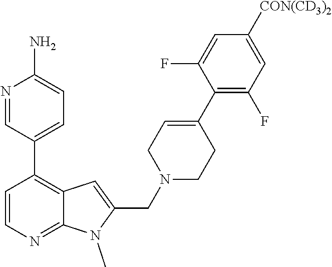 |
| 1z25 | 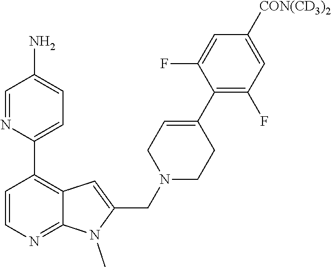 |
| 1z27 | 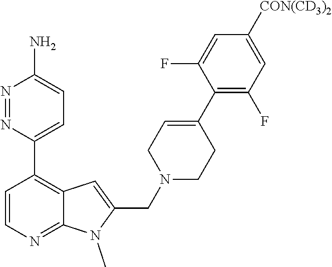 |
| 1z28 | 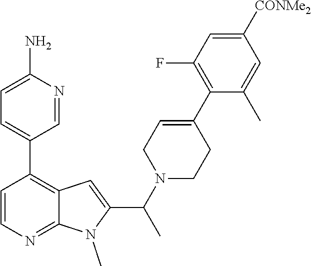 |
| 1z29 | 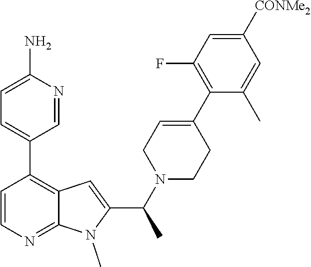 |
| 1z30 | 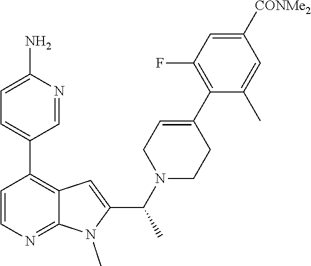 |
| 1z31 | 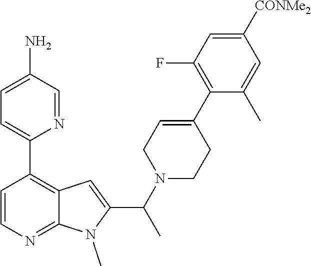 |
| 1z32 | 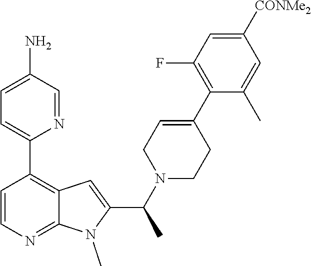 |
| 1z33 | 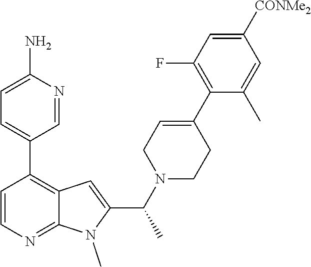 |
| 1z34 | 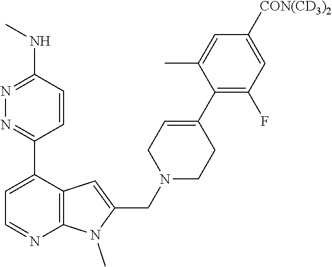 |
| 1z35 | 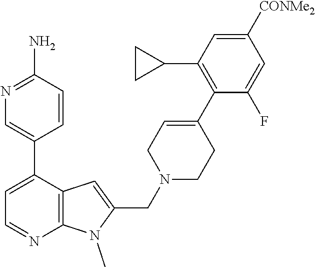 |
| 1z36 | 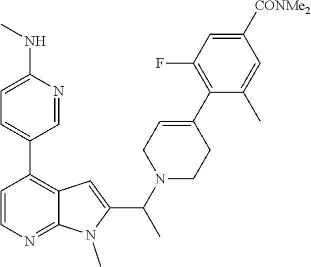 |
| 1z37 | 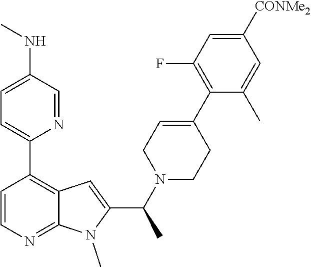 |
| 1z38 | 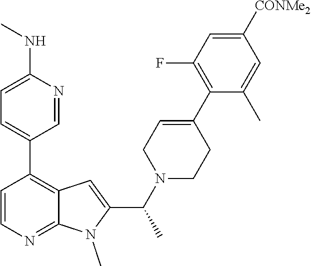 |
| 1z39 | 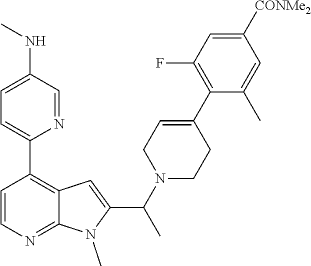 |
| 1z40 | 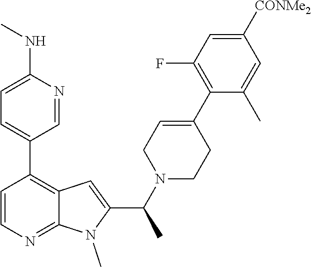 |
| 1z41 | 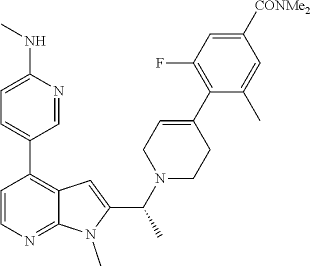 |
| 1z42 | 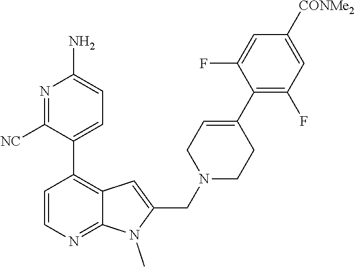 |
| 1z43 | 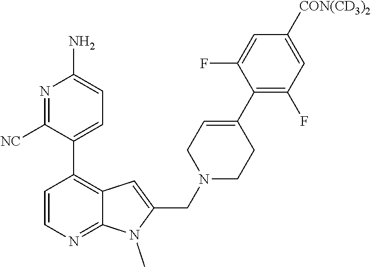 |
| 1z44 | 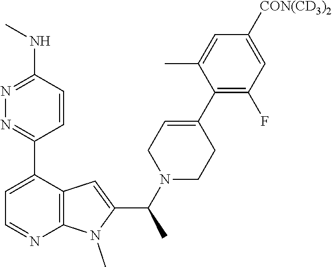 |
| 1z45 | 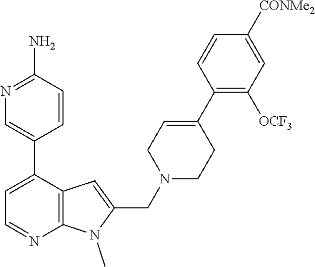 |

### Moiety A and Moiety L

In some embodiments, A is CO.

In some embodiments, A is

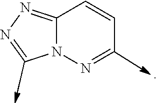

In some embodiments, L is selected from:

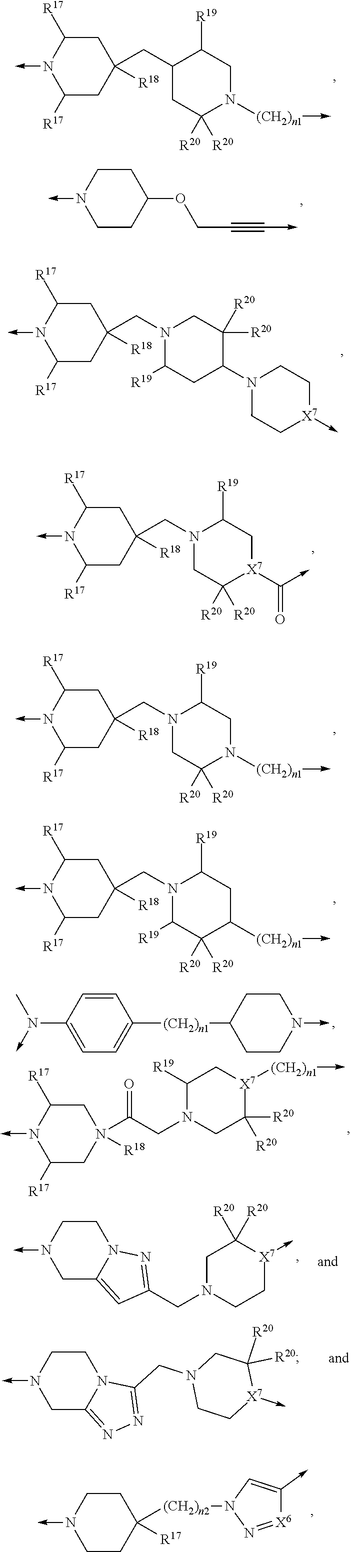

wherein each n1 is independently selected from 0 and 1;
n2 is selected from 1 and 2;
R¹⁷ is selected from hydrogen and fluoro;
R¹⁸ is hydrogen;
each R¹⁹ is independently selected from hydrogen, methyl, —CF₃, and fluoro;
each R²⁰ is independently selected from hydrogen and fluoro; and
X⁶ is CH or N.

In some embodiments, L is selected from:

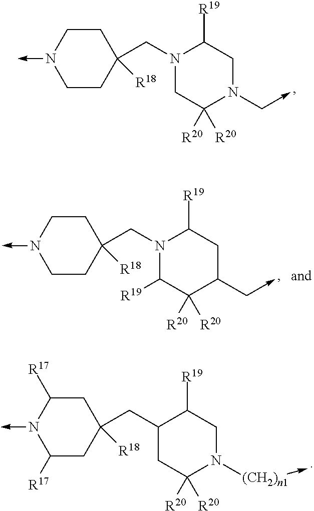

In some embodiments, L is selected from:

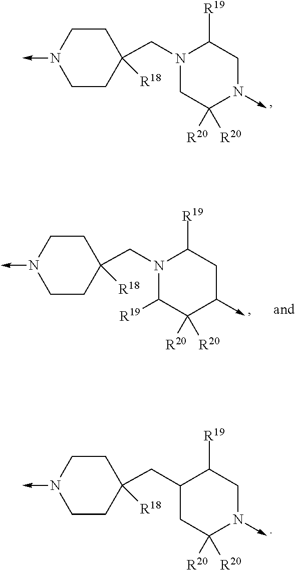

In some embodiments, L is

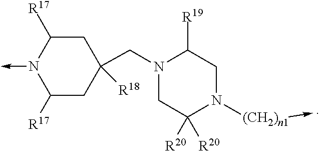

In some embodiments, L is

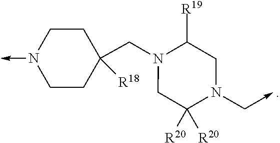

In some embodiments, L is

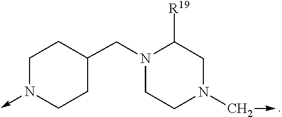

In some embodiments, L is

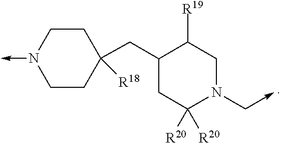

In some embodiments, L is

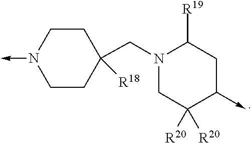

In some embodiments, L is

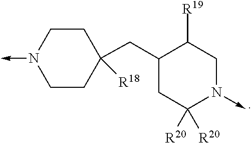

In some embodiments, L is

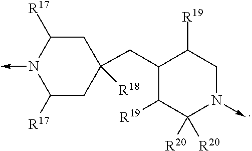

In some embodiments, L is

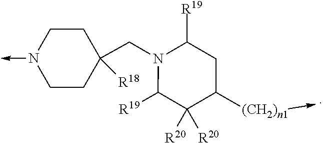

In some embodiments, L is

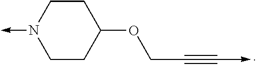

In some embodiments, L is

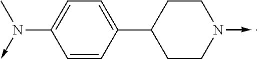

In some embodiments, at least one of the R¹⁷, R¹⁸, R¹⁹, and R²⁰ of L moiety is not hydrogen.

In some embodiments, R¹⁷ is hydrogen and at least one of the R¹⁸, R¹⁹, and R²⁰ of L moiety is not hydrogen.

In some embodiments, A-L is selected from:

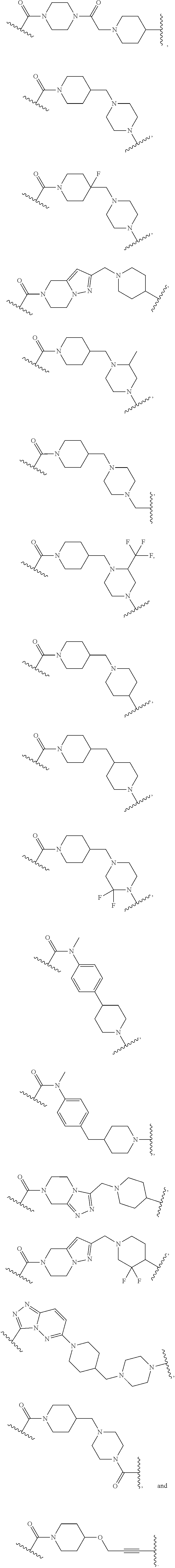

In some embodiments, L is:

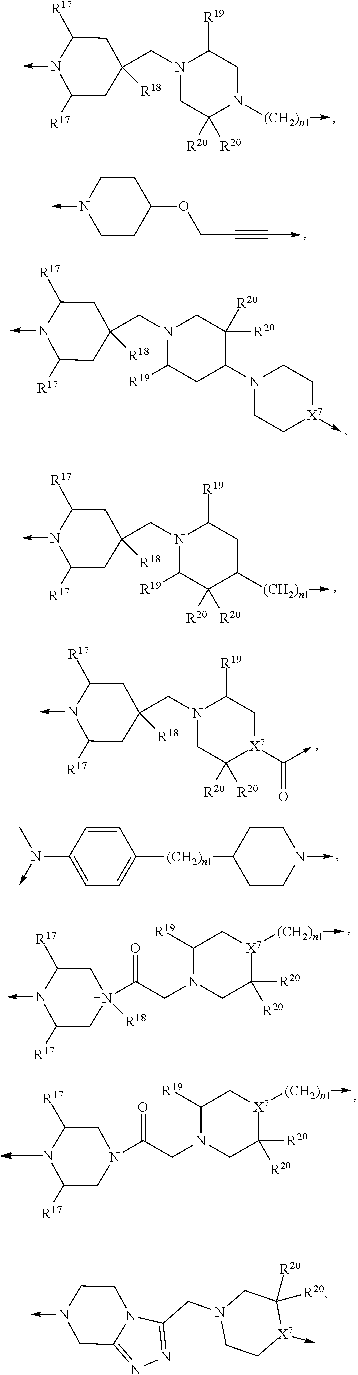

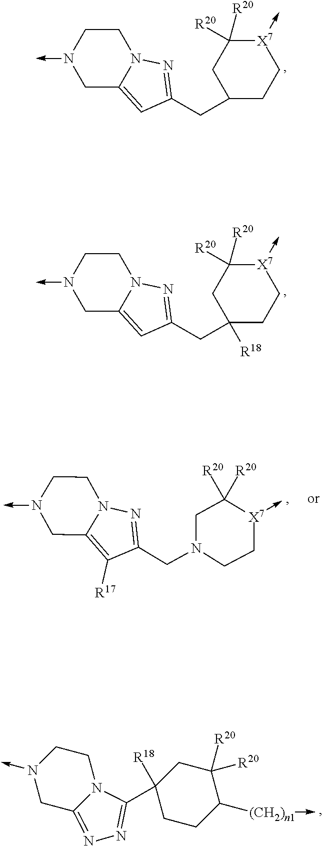

wherein
   R¹⁷ is selected from hydrogen, chloro, and fluoro;
   R¹⁸ is selected from hydrogen, fluoro, and hydroxy;
   R¹⁹ is selected from hydrogen and C₁₋₄alkyl, wherein said C₁₋₄alkyl may optionally be substituted one or more times with fluoro;
   R²⁰ is independently selected from hydrogen and fluoro;
   n1 is selected from 0 and 1; and
   X⁷ is selected from N and CH.

In some embodiments, L is:

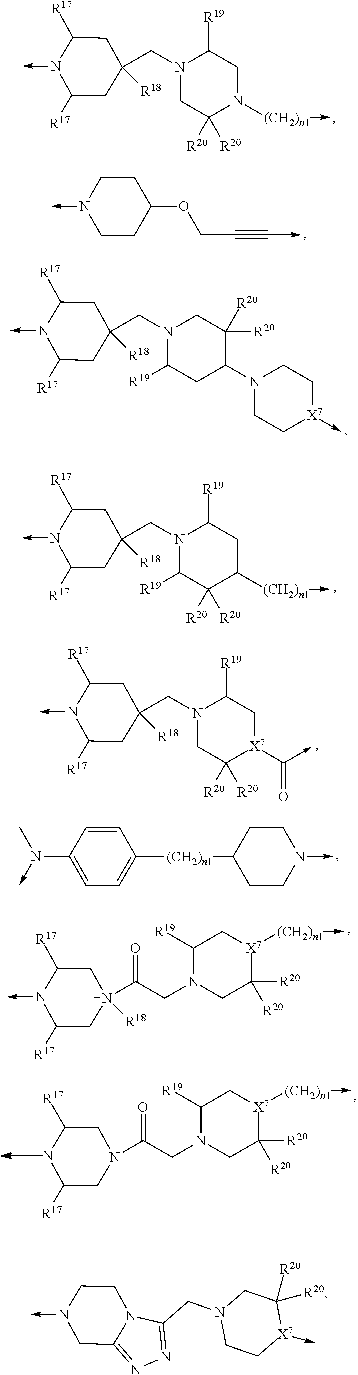

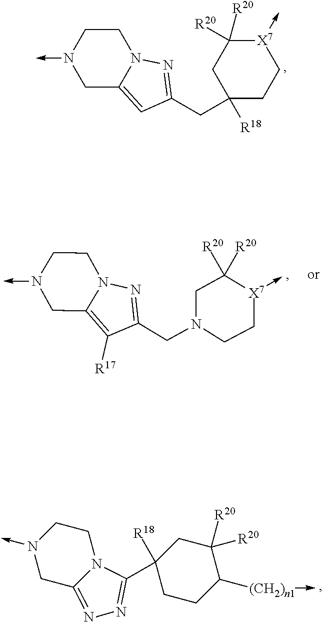

wherein
   R¹⁷ is selected from hydrogen, chloro, and fluoro;
   R¹⁸ is selected from hydrogen, fluoro, and hydroxy;
   R¹⁹ is selected from hydrogen and C₁₋₄alkyl, wherein said C₁₋₄alkyl may optionally be substituted one or more times with fluoro;
   R²⁰ is independently selected from hydrogen and fluoro;
   n1 is selected from 0 and 1; and
   X⁷ is selected from N and CH.

In some embodiments, L is

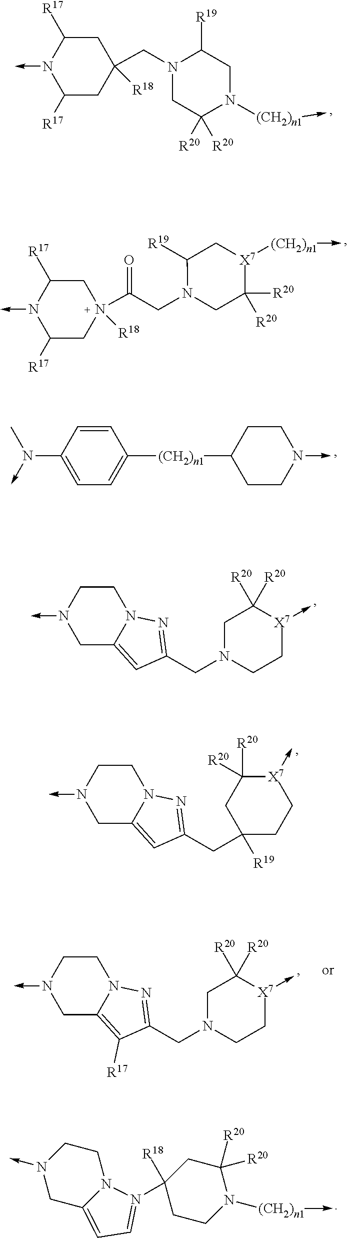

In some embodiments, L is

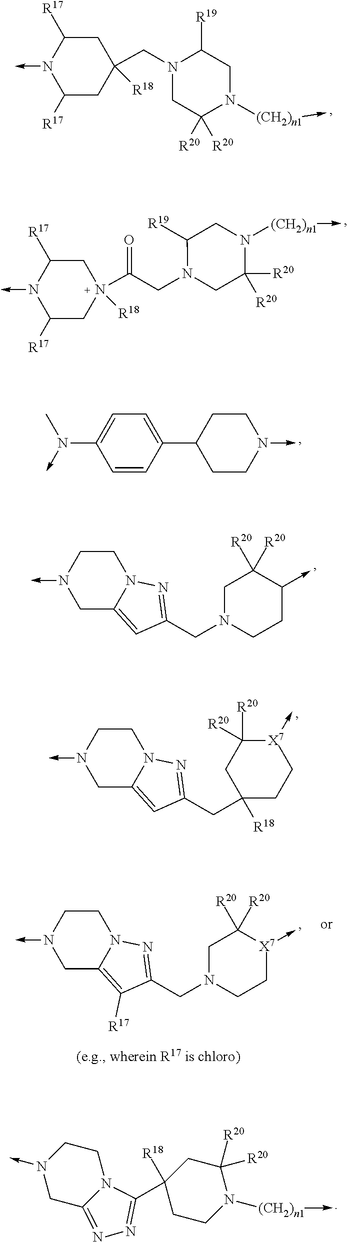

In some embodiments, L is selected from:

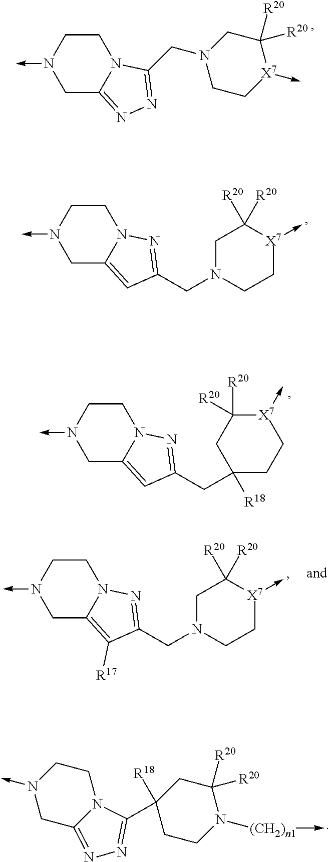

In some embodiments, L is a moiety as described herein,
R¹⁷ is selected from hydrogen and chloro;
R¹⁸ is selected from hydrogen and hydroxy;
R¹⁹ is selected from hydrogen and C₁₋₄alkyl;
R²⁰ is hydrogen;
n1 is 0; and
X⁷ is selected from N and CH.

### Moisty LBM

In some embodiments, LBM is selected from:

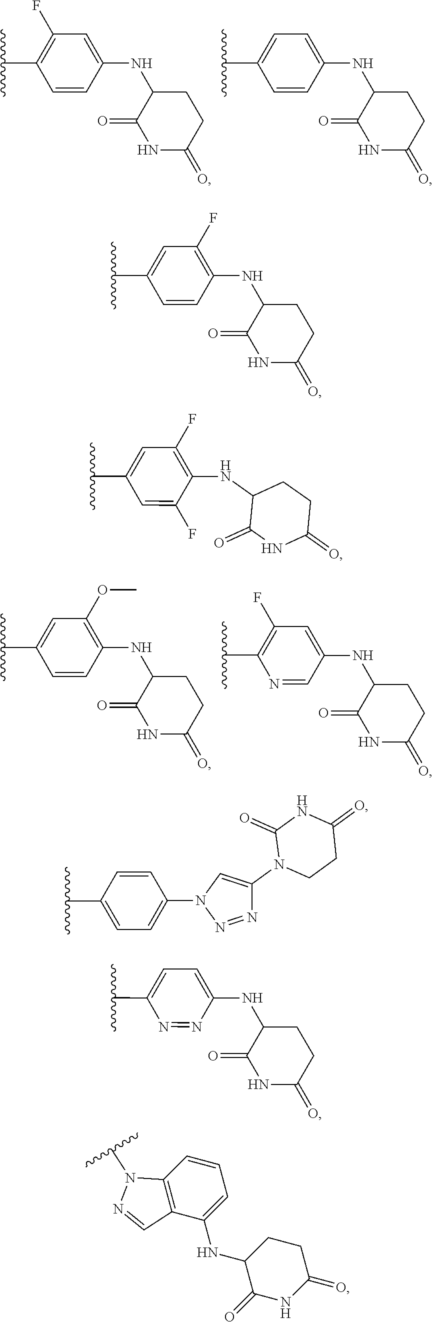

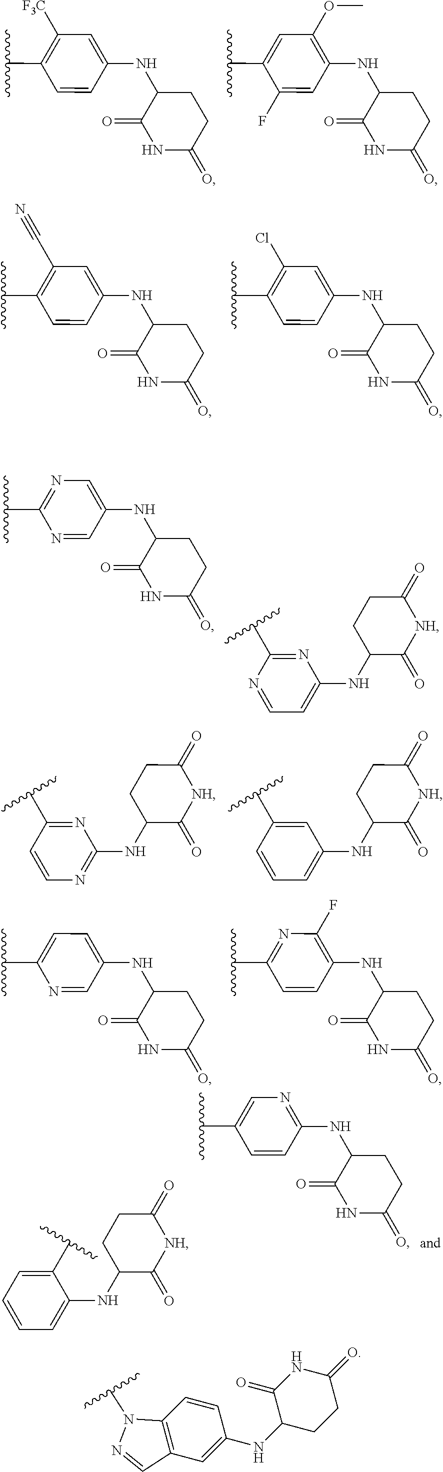

In some embodiments, LBM is selected from

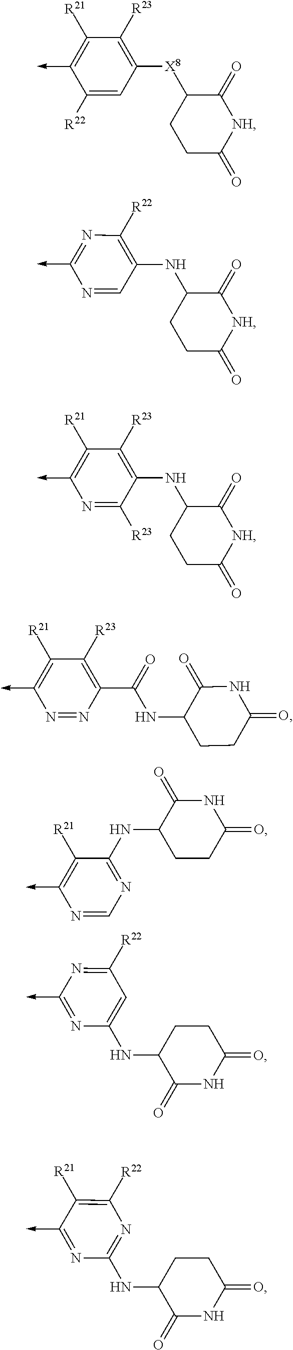

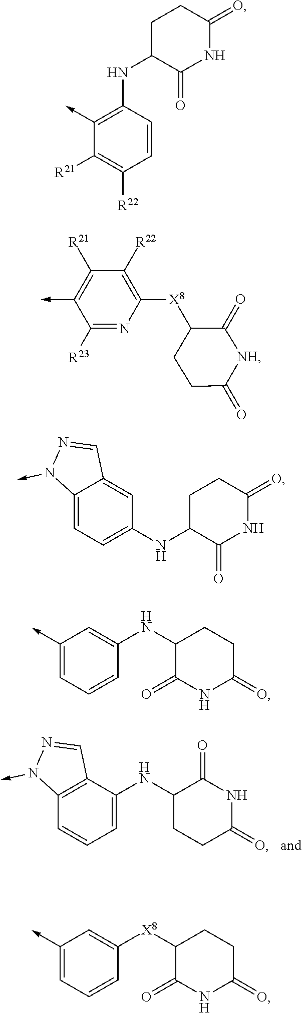

wherein
X⁸ is selected from O, C (O) NH, and NH;
R²¹ is selected from hydrogen, fluoro, chloro, cyano, C₁₋₄alkyl and trifluoromethyl;
R²² is selected from hydrogen and fluoro; and
R²³ is selected from hydrogen, halogen, C₁₋₄alkyl, and C₁₋₄alkoxy.

In some embodiments, LBM is

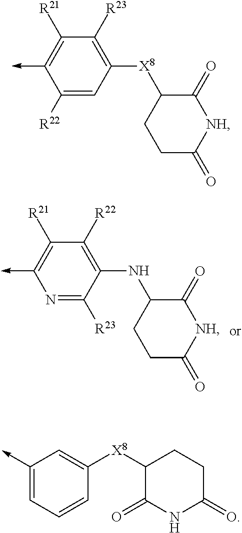

In some embodiments, LBM is

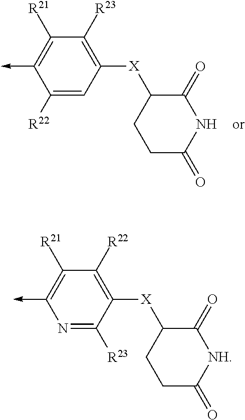

In some embodiments, LBM is

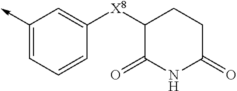

wherein X⁸ is selected from C (O) NH and NH.

In some embodiments, LBM is

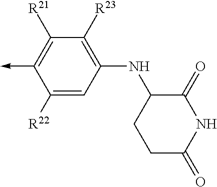

In some embodiments, LBM is a moiety as described herein,
X⁸ is NH;
R²¹ is selected from hydrogen and fluoro;
R²² is selected from hydrogen and fluoro; and
R²³ is selected from hydrogen and C₁₋₄alkoxy.

### Moiety A-L-LBM

In some embodiments,
A is CO₃
L is:

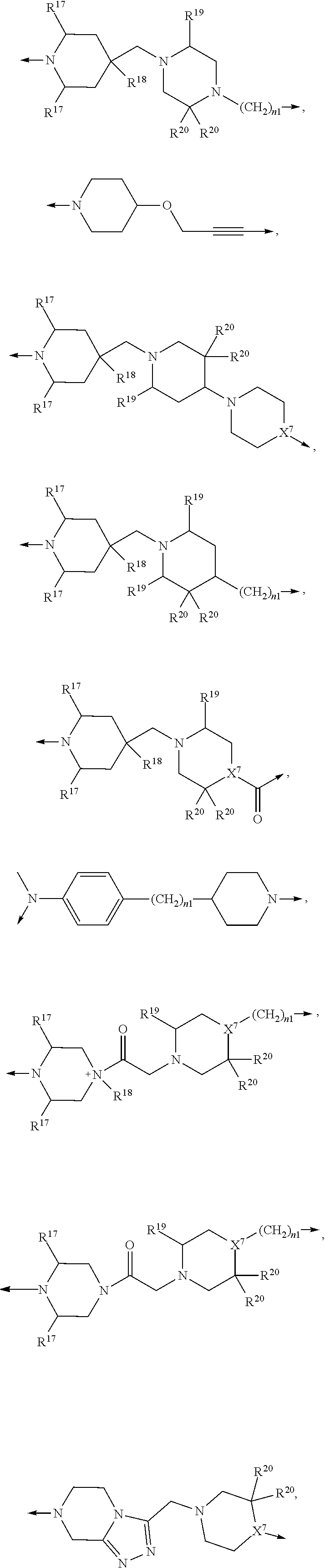

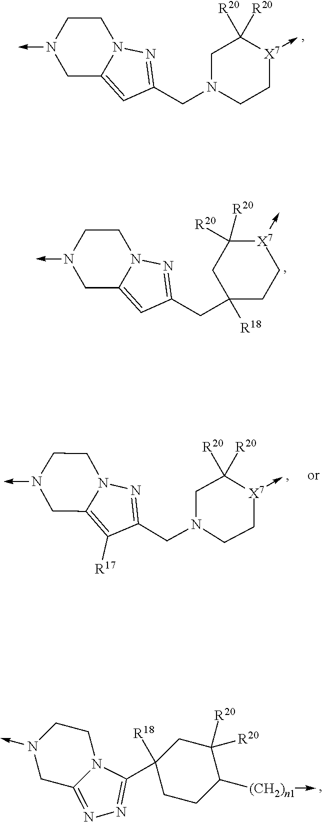

wherein
   R¹⁷ is selected from hydrogen, chloro, and fluoro;
   R¹⁸ is selected from hydrogen, fluoro, and hydroxy;
   R¹⁹ is selected from hydrogen and C₁₋₄alkyl, wherein said C₁₋₄alkyl may optionally be substituted one or more times with fluoro;
   R²⁰ is independently selected from hydrogen and fluoro;
   n1 is selected from 0 and 1;
   X⁷ is selected from N and CH; and
   LBM is selected from

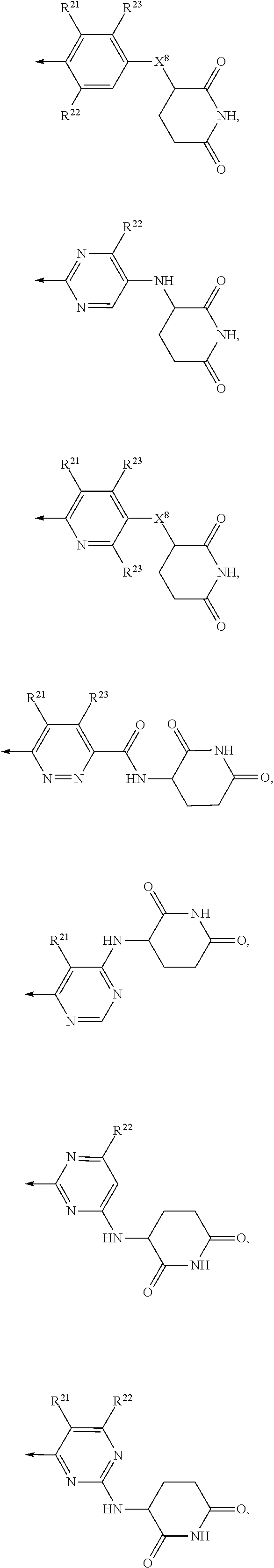

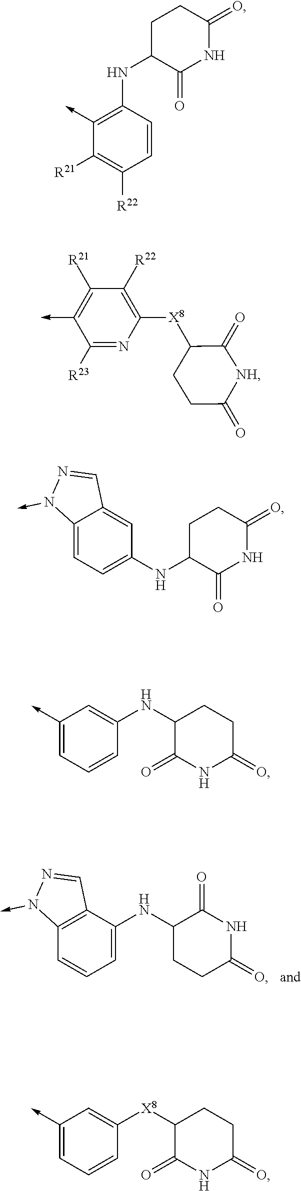

wherein
   X⁸ is selected from O, C (O) NH, and NH;
   R²¹ is selected from hydrogen, fluoro, chloro, cyano, C₁₋₄alkyl and trifluoromethyl;
   R²² is selected from hydrogen and fluoro;
   R²³ is selected from hydrogen, halogen, C₁₋₄alkyl, and C₁₋₄alkoxy.

In a further embodiment the disclosure provides a compound as above wherein A-L-LBM is

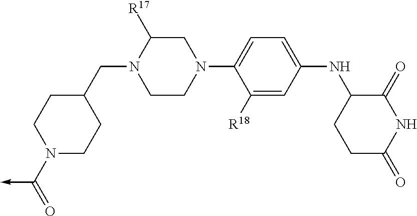

wherein R¹⁷ and R¹⁸ are as defined above.

In some embodiments, -A-L-LBM is

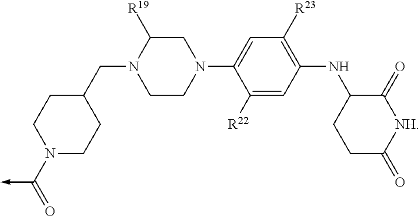

In some embodiments, -A-L-LBM is

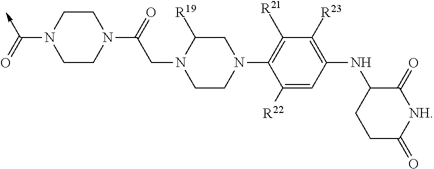

In some embodiments, -A-L-LBM is

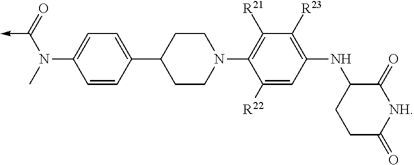

In some embodiments, -A-L-LBM is

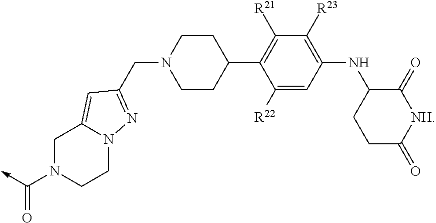

In some embodiments, -A-L-LBM is

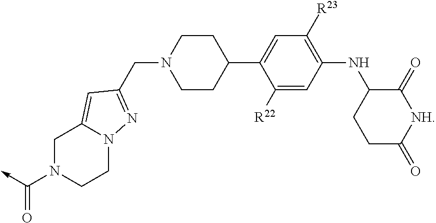

In certain embodiments, provided is a compound selected from Table 1, or a pharmaceutically acceptable salt or stereoisomer thereof:

TABLE 1

| Example | Structure |
|---|---|
| 2a1 | 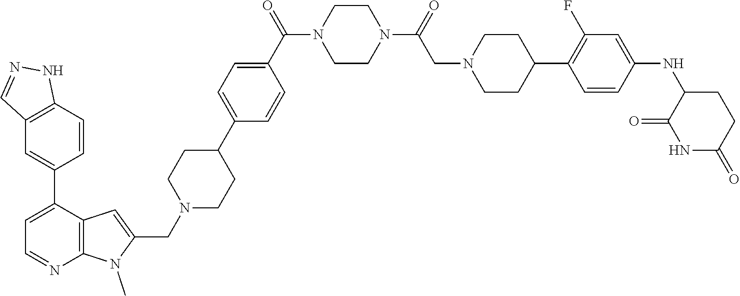 [SMILES: Cn1c(CN2CCC(c3ccc(C(=O)N4CCN(C(=O)CN5CCC(c6ccc(NC7CCC(=O)NC7=O)cc6F)CC5)CC4)cc3)CC2)cc2c(-c3ccc4[nH]ncc4c3)ccnc21] |
| 2a2 | 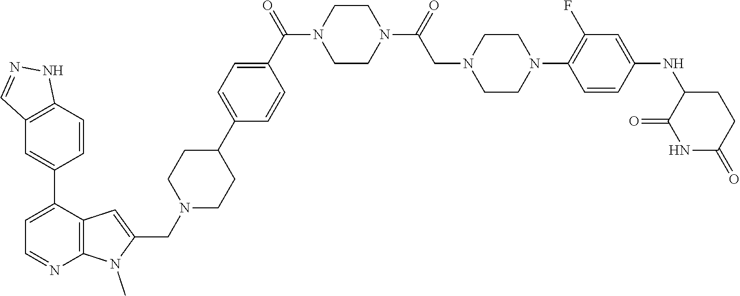 [SMILES: Cn1c(CN2CCC(c3ccc(C(=O)N4CCN(C(=O)CN5CCN(c6ccc(NC7CCC(=O)NC7=O)cc6F)CC5)CC4)cc3)CC2)cc2c(-c3ccc4[nH]ncc4c3)ccnc21] |
| 2b1 | 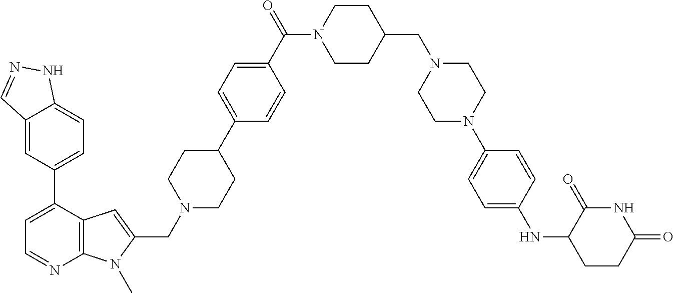 [SMILES: Cn1c(CN2CCC(c3ccc(C(=O)N4CCC(CN5CCN(c6ccc(NC7CCC(=O)NC7=O)cc6)CC5)CC4)cc3)CC2)cc2c(-c3ccc4[nH]ncc4c3)ccnc21] |
| 2b2 | 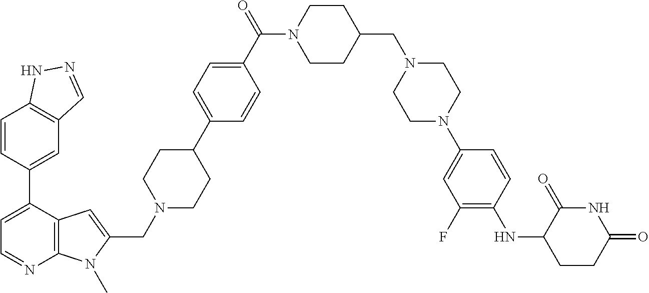 [SMILES: Cn1c(CN2CCC(c3ccc(C(=O)N4CCC(CN5CCN(c6ccc(NC7CCC(=O)NC7=O)c(F)c6)CC5)CC4)cc3)CC2)cc2c(-c3ccc4[nH]ncc4c3)ccnc21] |
| 2b3 | 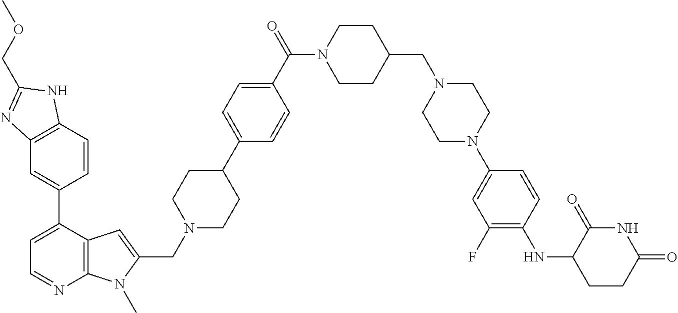 [SMILES: COCc1nc2cc(-c3ccnc4c3cc(CN3CCC(c5ccc(C(=O)N6CCC(CN7CCN(c8ccc(NC9CCC(=O)NC9=O)c(F)c8)CC7)CC6)cc5)CC3)n4C)ccc2[nH]1] |
| 2c1 | 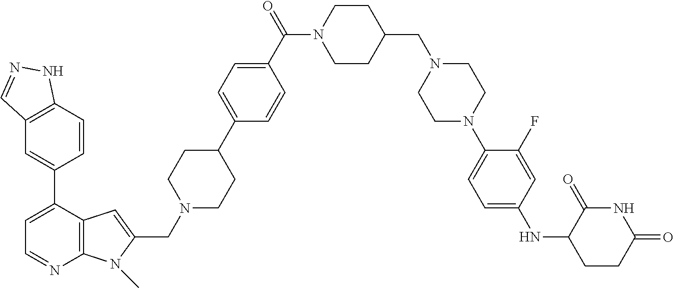 [SMILES: Cn1c(CN2CCC(c3ccc(C(=O)N4CCC(CN5CCN(c6ccc(NC7CCC(=O)NC7=O)cc6F)CC5)CC4)cc3)CC2)cc2c(-c3ccc4[nH]ncc4c3)ccnc21] |
| 2c2 | 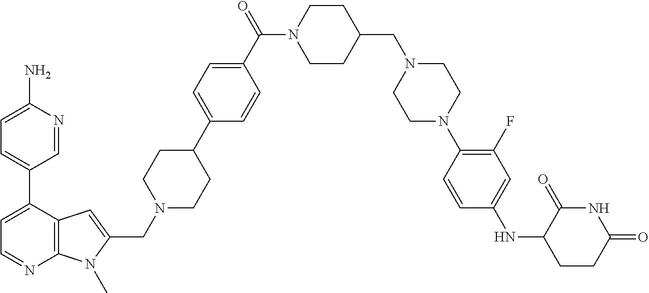 [SMILES: Cn1c(CN2CCC(c3ccc(C(=O)N4CCC(CN5CCN(c6ccc(NC7CCC(=O)NC7=O)cc6F)CC5)CC4)cc3)CC2)cc2c(-c3ccc(N)nc3)ccnc21] |
| 2c3 | 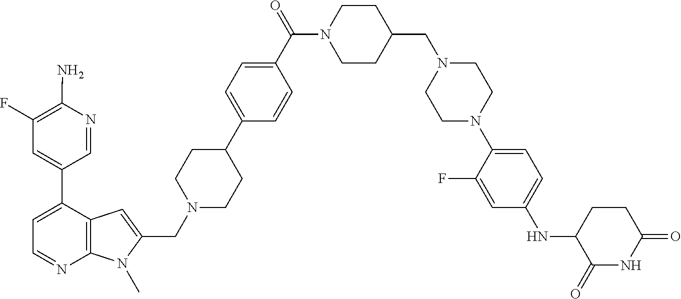 [SMILES: Cn1c(CN2CCC(c3ccc(C(=O)N4CCC(CN5CCN(c6ccc(NC7CCC(=O)NC7=O)cc6F)CC5)CC4)cc3)CC2)cc2c(-c3cnc(N)c(F)c3)ccnc21] |
| 2c4 | 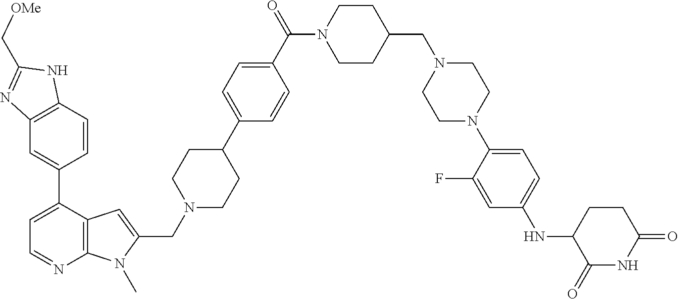 [SMILES: COCc1nc2cc(-c3ccnc4c3cc(CN3CCC(c5ccc(C(=O)N6CCC(CN7CCN(c8ccc(NC9CCC(=O)NC9=O)cc8F)CC7)CC6)cc5)CC3)n4C)ccc2[nH]1] |
| 2c5 | 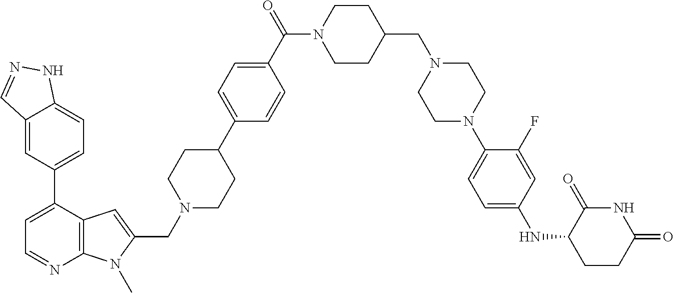 [SMILES: Cn1c(CN2CCC(c3ccc(C(=O)N4CCC(CN5CCN(c6ccc(N[C@H]7CCC(=O)NC7=O)cc6F)CC5)CC4)cc3)CC2)cc2c(-c3ccc4[nH]ncc4c3)ccnc21] |
| 2c6 | 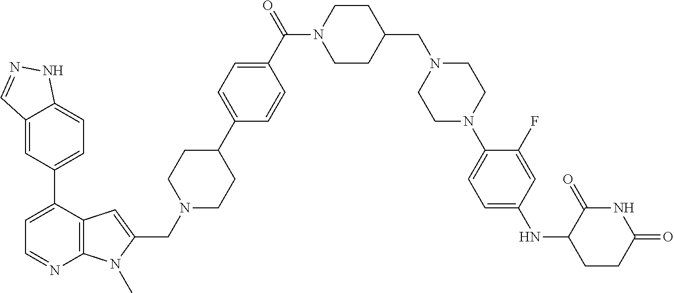 [SMILES: Cn1c(CN2CCC(c3ccc(C(=O)N4CCC(CN5CCN(c6ccc(NC7CCC(=O)NC7=O)cc6F)CC5)CC4)cc3)CC2)cc2c(-c3ccc4[nH]ncc4c3)ccnc21] |
| 2c7 | 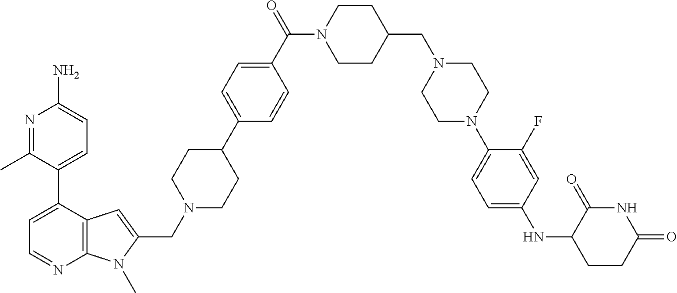 [SMILES: Cc1nc(N)ccc1-c1ccnc2c1cc(CN1CCC(c3ccc(C(=O)N4CCC(CN5CCN(c6ccc(NC7CCC(=O)NC7=O)cc6F)CC5)CC4)cc3)CC1)n2C] |
| 2c8 | 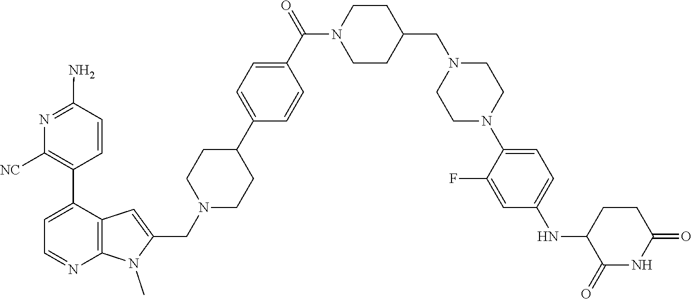 [SMILES: Cn1c(CN2CCC(c3ccc(C(=O)N4CCC(CN5CCN(c6ccc(NC7CCC(=O)NC7=O)cc6F)CC5)CC4)cc3)CC2)cc2c(-c3ccc(N)nc3C#N)ccnc21] |
| 2c9 | 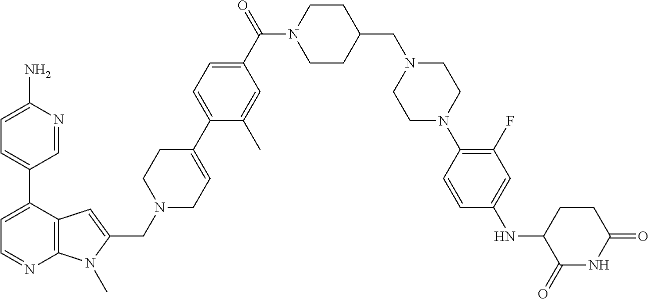 [SMILES: Cc1cc(C(=O)N2CCC(CN3CCN(c4ccc(NC5CCC(=O)NC5=O)cc4F)CC3)CC2)ccc1C1=CCN(Cc2cc3c(-c4ccc(N)nc4)ccnc3n2C)CC1] |
| 2c10 | 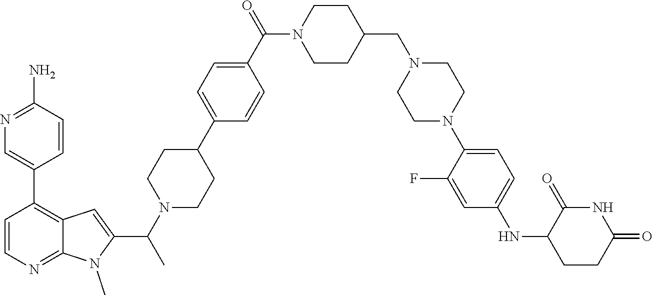 [SMILES: CC(c1cc2c(-c3ccc(N)nc3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c11 | 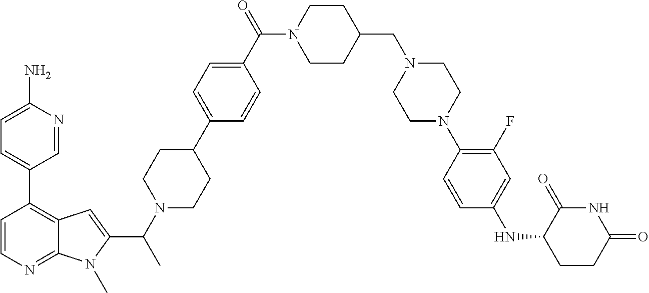 [SMILES: CC(c1cc2c(-c3ccc(N)nc3)ccnc2n1C)N1CCC(c2ccc(C(=O)N3CCC(CN4CCN(c5ccc(N[C@H]6CCC(=O)NC6=O)cc5F)CC4)CC3)cc2)CC1] |
| 2c12 | 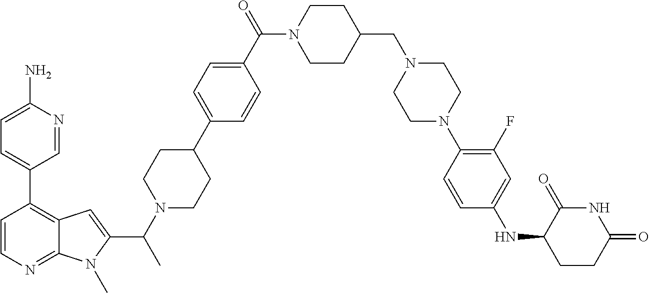 [SMILES: CC(c1cc2c(-c3ccc(N)nc3)ccnc2n1C)N1CCC(c2ccc(C(=O)N3CCC(CN4CCN(c5ccc(N[C@@H]6CCC(=O)NC6=O)cc5F)CC4)CC3)cc2)CC1] |
| 2c13 | 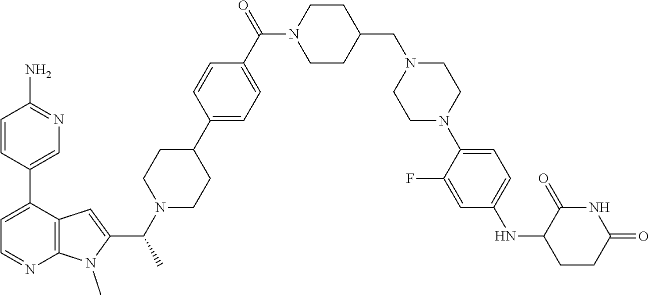 [SMILES: C[C@H](c1cc2c(-c3ccc(N)nc3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c14 | 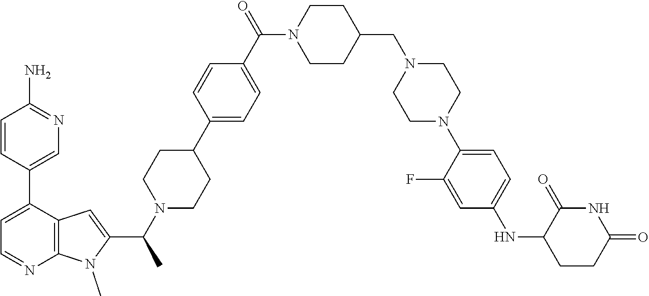 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c15 | 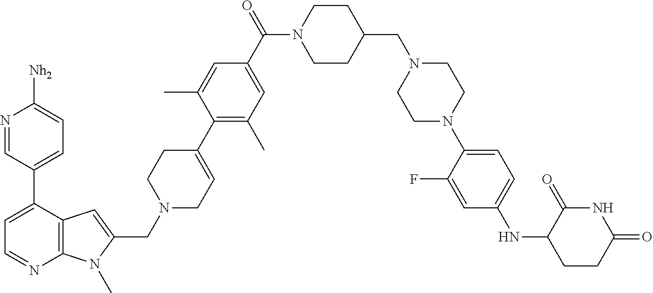 |
| 2c16 | 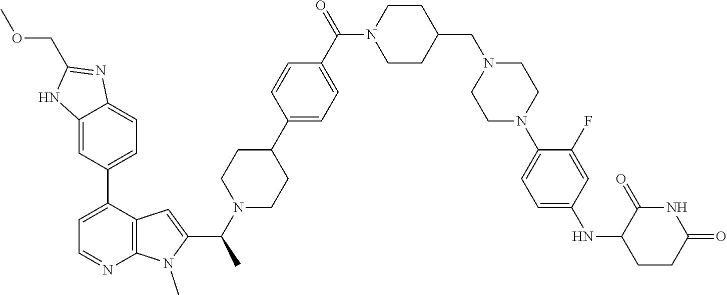 [SMILES: COCc1nc2ccc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)cc2[nH]1] |
| 2c17 | 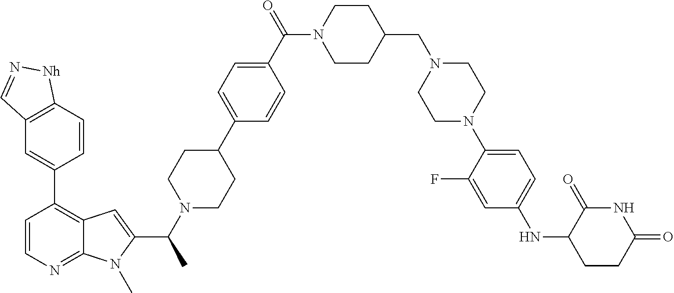 |
| 2c18 | 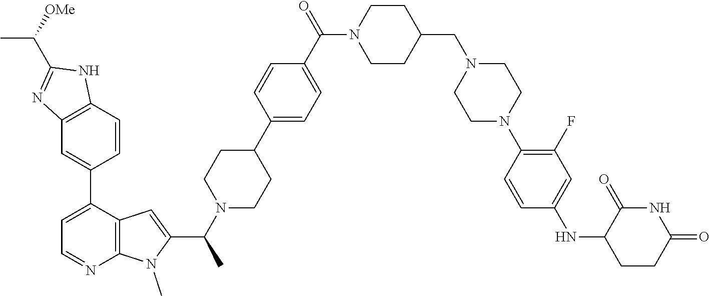 [SMILES: CO[C@@H](C)c1nc2cc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)ccc2[nH]1] |
| 2c19 | 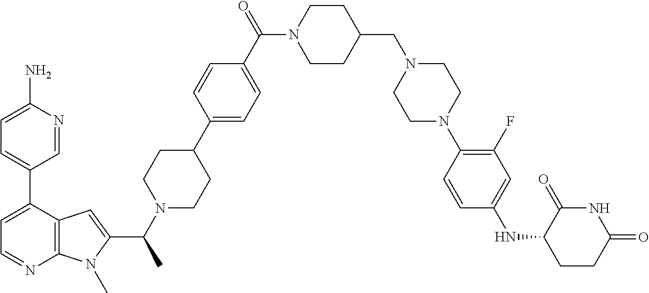 [SMILES: C[C@@H](c1cc2c(-c3ccc(N)nc3)ccnc2n1C)N1CCC(c2ccc(C(=O)N3CCC(CN4CCN(c5ccc(N[C@H]6CCC(=O)NC6=O)cc5F)CC4)CC3)cc2)CC1] |
| 2c20 | 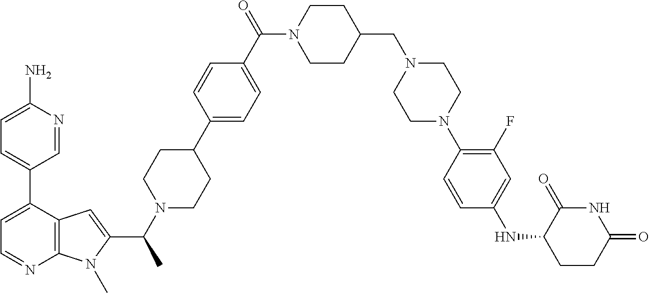 [SMILES: C[C@@H](c1cc2c(-c3ccc(N)nc3)ccnc2n1C)N1CCC(c2ccc(C(=O)N3CCC(CN4CCN(c5ccc(N[C@H]6CCC(=O)NC6=O)cc5F)CC4)CC3)cc2)CC1] |
| 2c21 | 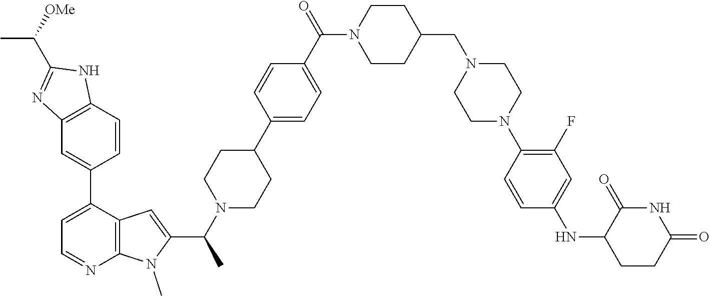 [SMILES: CO[C@@H](C)c1nc2cc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)ccc2[nH]1] |
| 2c22 | 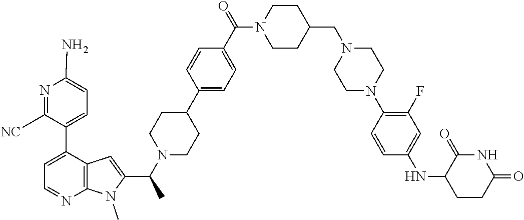 [SMILES: C[C@@H](c1cc2c(-c3ccc(N)nc3C#N)ccnc2n1C)N1CCC(c2ccc(C(=O)N3CCC(CN4CCN(c5ccc(NC6CCC(=O)NC6=O)cc5F)CC4)CC3)cc2)CC1] |
| 2c23 | 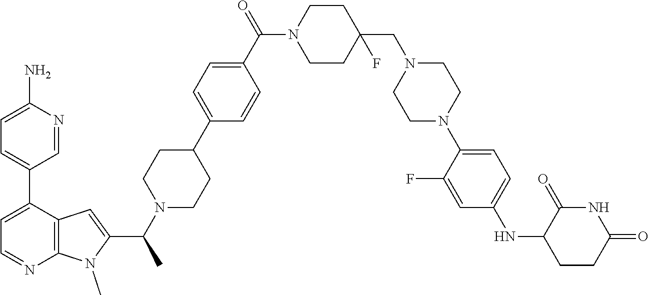 [SMILES: C[C@@H](c1cc2c(-c3ccc(N)nc3)ccnc2n1C)N1CCC(c2ccc(C(=O)N3CCC(F)(CN4CCN(c5ccc(NC6CCC(=O)NC6=O)cc5F)CC4)CC3)cc2)CC1] |
| 2c24 | 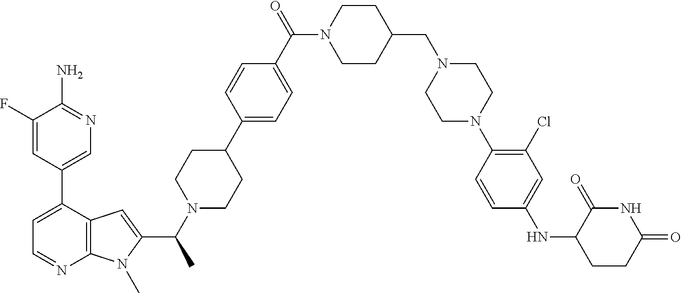 [SMILES: C[C@@H](c1cc2c(-c3cnc(N)c(F)c3)ccnc2n1C)N1CCC(c2ccc(C(=O)N3CCC(CN4CCN(c5ccc(NC6CCC(=O)NC6=O)cc5Cl)CC4)CC3)cc2)CC1] |
| 2c26 | 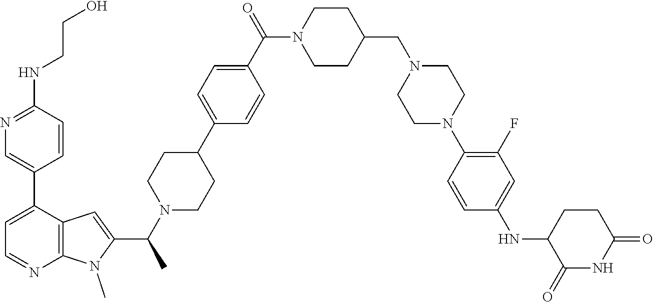 [SMILES: C[C@@H](c1cc2c(-c3ccc(NCCO)nc3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c27 | 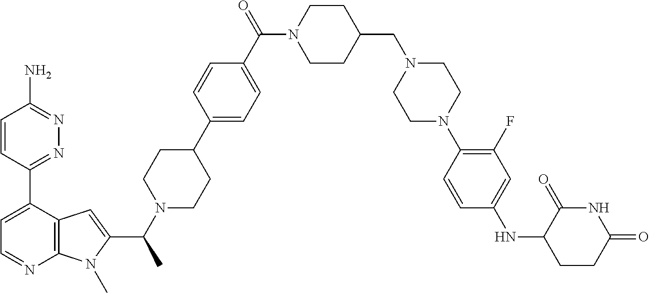 [SMILES: C[C@@H](c1cc2c(-c3ccc(N)nn3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c28 | 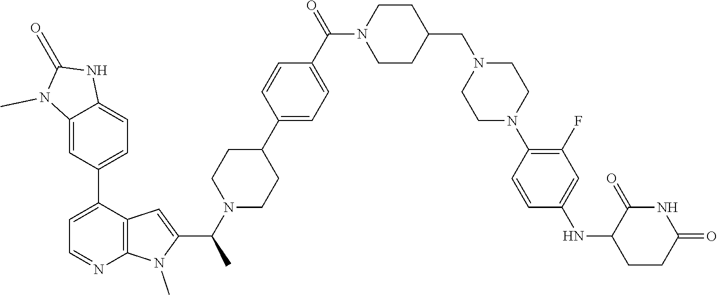 [SMILES: C[C@@H](c1cc2c(-c3ccc4[nH]c(=O)n(C)c4c3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c30 | 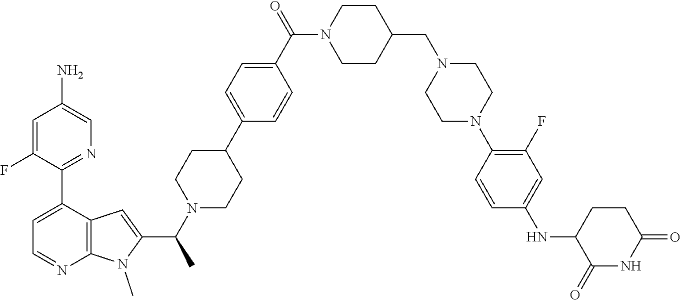 [SMILES: C[C@@H](c1cc2c(-c3ncc(N)cc3F)ccnc2n1C)N1CCC(c2ccc(C(=O)N3CCC(CN4CCN(c5ccc(NC6CCC(=O)NC6=O)cc5F)CC4)CC3)cc2)CC1] |
| 2c32 | 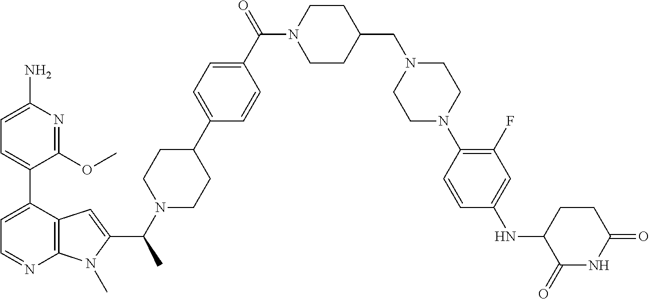 [SMILES: COc1nc(N)ccc1-c1ccnc2c1cc([C@H](C)N1CCC(c3ccc(C(=O)N4CCC(CN5CCN(c6ccc(NC7CCC(=O)NC7=O)cc6F)CC5)CC4)cc3)CC1)n2C] |
| 2c33 | 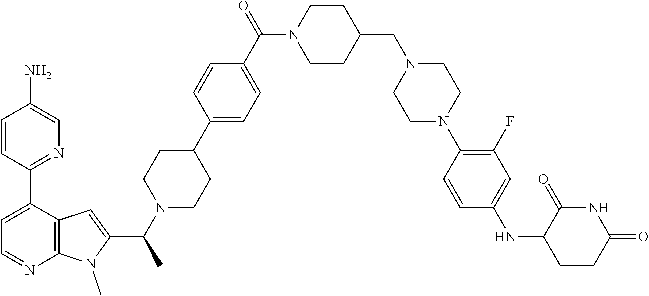 [SMILES: C[C@@H](c1cc2c(-c3ccc(N)cn3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c34 | 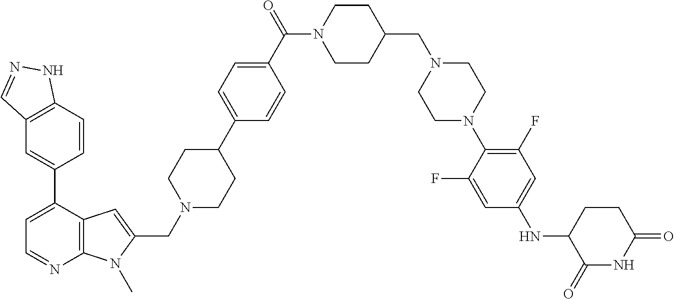 [SMILES: Cn1c(CN2CCC(c3ccc(C(=O)N4CCC(CN5CCN(c6c(F)cc(NC7CCC(=O)NC7=O)cc6F)CC5)CC4)cc3)CC2)cc2c(-c3ccc4[nH]ncc4c3)ccnc21] |
| 2c37 | 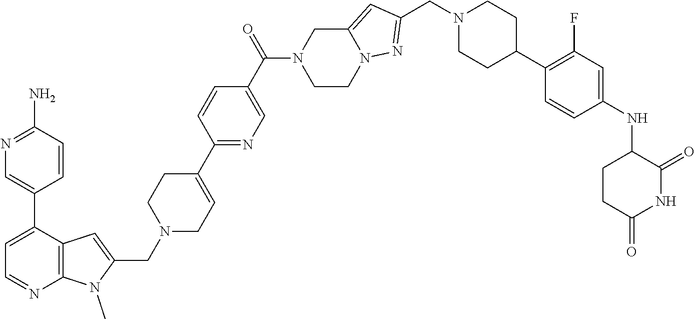 [SMILES: Cn1c(CN2CC=C(c3ccc(C(=O)N4CCn5nc(CN6CCC(c7ccc(NC8CCC(=O)NC8=O)cc7F)CC6)cc5C4)cn3)CC2)cc2c(-c3ccc(N)nc3)ccnc21] |
| 2c38 | 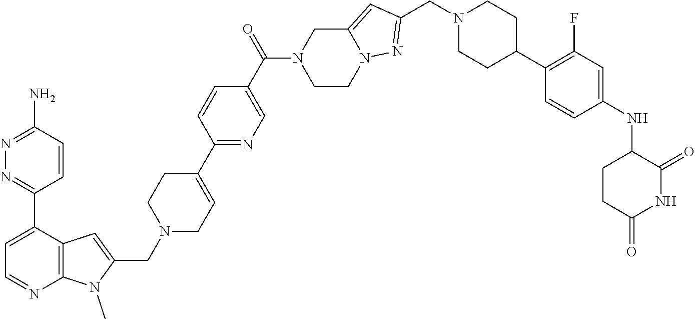 [SMILES: Cn1c(CN2CC=C(c3ccc(C(=O)N4CCn5nc(CN6CCC(c7ccc(NC8CCC(=O)NC8=O)cc7F)CC6)cc5C4)cn3)CC2)cc2c(-c3ccc(N)nn3)ccnc21] |
| 2c39 | 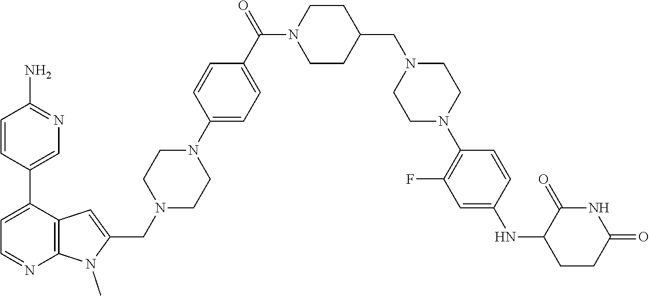 [SMILES: Cn1c(CN2CCN(c3ccc(C(=O)N4CCC(CN5CCN(c6ccc(NC7CCC(=O)NC7=O)cc6F)CC5)CC4)cc3)CC2)cc2c(-c3ccc(N)nc3)ccnc21] |
| 2c40 | 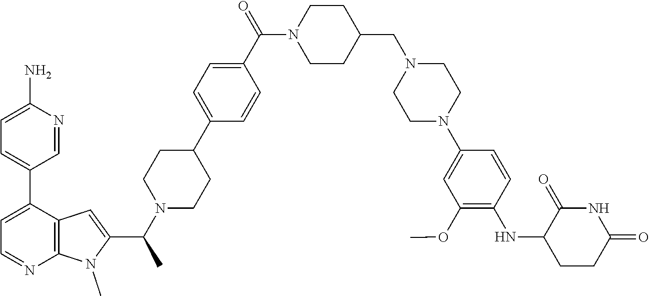 [SMILES: COc1cc(N2CCN(CC3CCN(C(=O)c4ccc(C5CCN([C@@H](C)c6cc7c(-c8ccc(N)nc8)ccnc7n6C)CC5)cc4)CC3)CC2)ccc1NC1CCC(=O)NC1=O] |
| 2c41 | 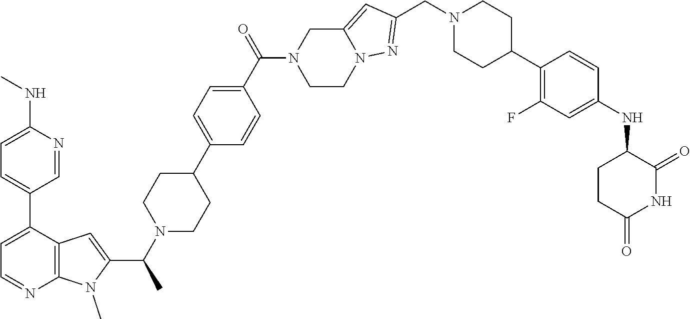 [SMILES: CNc1ccc(-c2ccnc3c2cc([C@H](C)N2CCC(c4ccc(C(=O)N5CCn6nc(CN7CCC(c8ccc(N[C@@H]9CCC(=O)NC9=O)cc8F)CC7)cc6C5)cc4)CC2)n3C)cn1] |
| 2c42 | 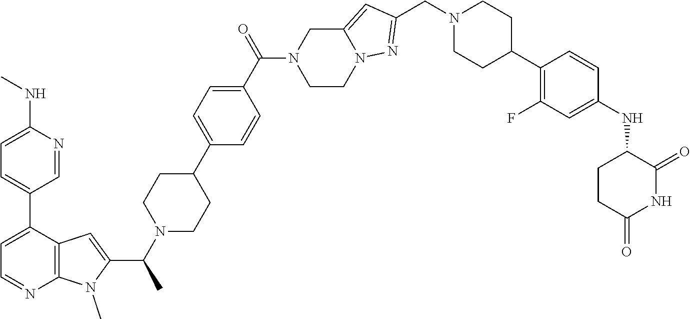 [SMILES: CNc1ccc(-c2ccnc3c2cc([C@H](C)N2CCC(c4ccc(C(=O)N5CCn6nc(CN7CCC(c8ccc(N[C@H]9CCC(=O)NC9=O)cc8F)CC7)cc6C5)cc4)CC2)n3C)cn1] |
| 2c43 | 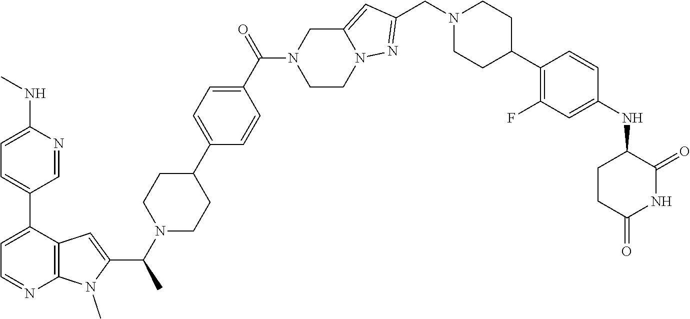 [SMILES: CNc1ccc(-c2ccnc3c2cc([C@H](C)N2CCC(c4ccc(C(=O)N5CCn6nc(CN7CCC(c8ccc(N[C@@H]9CCC(=O)NC9=O)cc8F)CC7)cc6C5)cc4)CC2)n3C)cn1] |
| 2c44 | 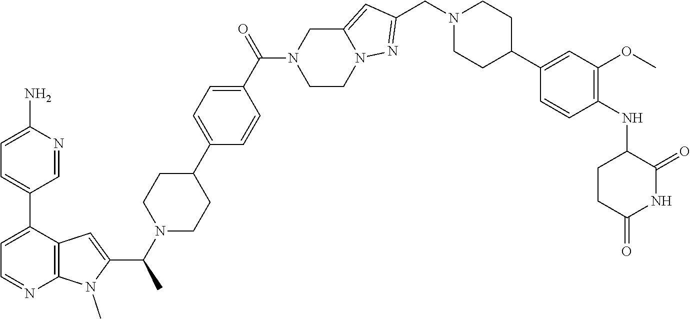 [SMILES: COc1cc(C2CCN(Cc3cc4n(n3)CCN(C(=O)c3ccc(C5CCN([C@@H](C)c6cc7c(-c8ccc(N)nc8)ccnc7n6C)CC5)cc3)C4)CC2)ccc1NC1CCC(=O)NC1=O] |
| 2c45 | 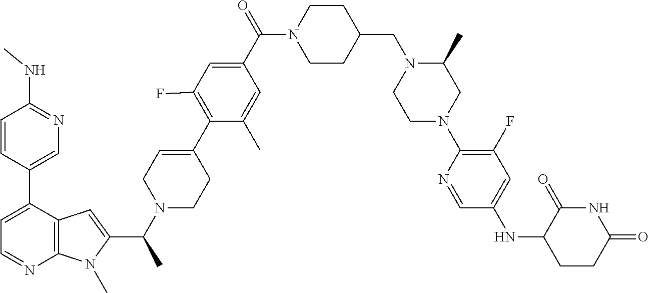 [SMILES: CNc1ccc(-c2ccnc3c2cc([C@H](C)N2CC=C(c4c(C)cc(C(=O)N5CCC(CN6CCN(c7ncc(NC8CCC(=O)NC8=O)cc7F)C[C@@H]6C)CC5)cc4F)CC2)n3C)cn1] |
| 2c46 | 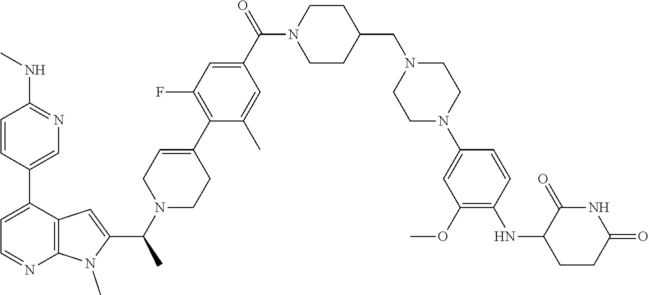 [SMILES: CNc1ccc(-c2ccnc3c2cc([C@H](C)N2CC=C(c4c(C)cc(C(=O)N5CCC(CN6CCN(c7ccc(NC8CCC(=O)NC8=O)c(OC)c7)CC6)CC5)cc4F)CC2)n3C)cn1] |
| 2c47 | 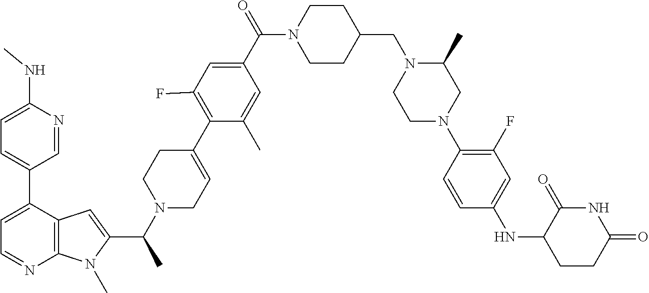 [SMILES: CNc1ccc(-c2ccnc3c2cc([C@H](C)N2CC=C(c4c(C)cc(C(=O)N5CCC(CN6CCN(c7ccc(NC8CCC(=O)NC8=O)cc7F)C[C@@H]6C)CC5)cc4F)CC2)n3C)cn1] |
| 2c48 | 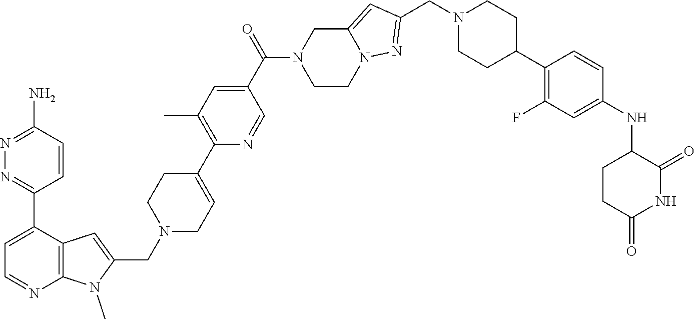 [SMILES: Cc1cc(C(=O)N2CCn3nc(CN4CCC(c5ccc(NC6CCC(=O)NC6=O)cc5F)CC4)cc3C2)cnc1C1=CCN(Cc2cc3c(-c4ccc(N)nn4)ccnc3n2C)CC1] |
| 2c49 | 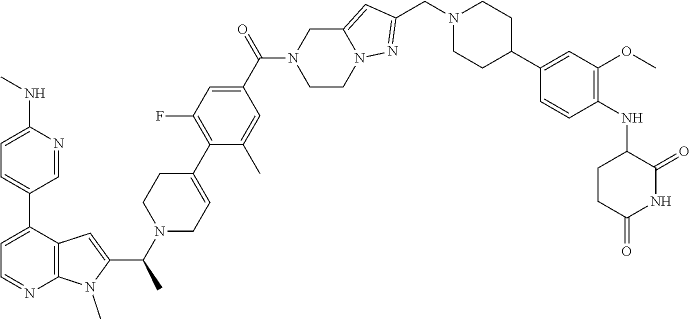 [SMILES: CNc1ccc(-c2ccnc3c2cc([C@H](C)N2CC=C(c4c(C)cc(C(=O)N5CCn6nc(CN7CCC(c8ccc(NC9CCC(=O)NC9=O)c(OC)c8)CC7)cc6C5)cc4F)CC2)n3C)cn1] |
| 2c52 | 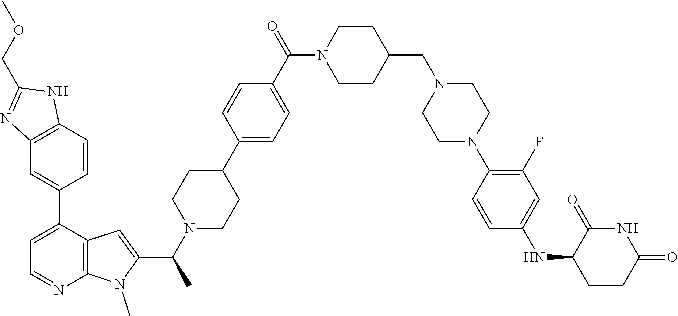 [SMILES: COCc1nc2cc(-c3ccnc4c3cc([C@H](C)N3CCC(c5ccc(C(=O)N6CCC(CN7CCN(c8ccc(N[C@@H]9CCC(=O)NC9=O)cc8F)CC7)CC6)cc5)CC3)n4C)ccc2[nH]1] |
| 2c53 | 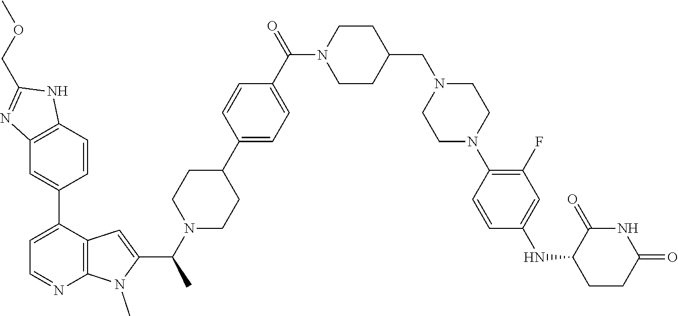 [SMILES: COCc1nc2cc(-c3ccnc4c3cc([C@H](C)N3CCC(c5ccc(C(=O)N6CCC(CN7CCN(c8ccc(N[C@H]9CCC(=O)NC9=O)cc8F)CC7)CC6)cc5)CC3)n4C)ccc2[nH]1] |
| 2c54 | 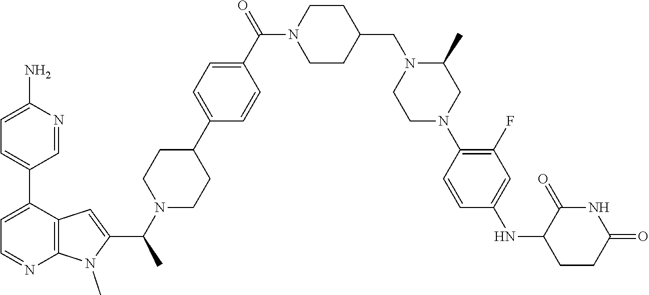 [SMILES: C[C@H]1CN(c2ccc(NC3CCC(=O)NC3=O)cc2F)CCN1CC1CCN(C(=O)c2ccc(C3CCN([C@@H](C)c4cc5c(-c6ccc(N)nc6)ccnc5n4C)CC3)cc2)CC1] |
| 2c55 | 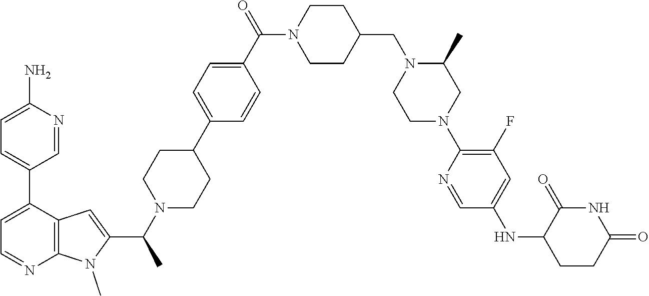 [SMILES: C[C@H]1CN(c2ncc(NC3CCC(=O)NC3=O)cc2F)CCN1CC1CCN(C(=O)c2ccc(C3CCN([C@@H](C)c4cc5c(-c6ccc(N)nc6)ccnc5n4C)CC3)cc2)CC1] |
| 2c56 | 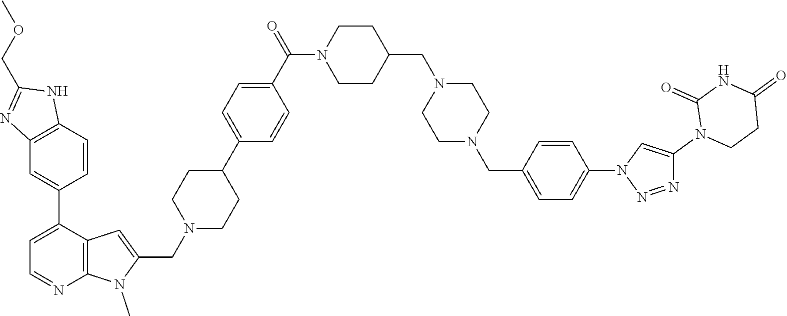 [SMILES: COCc1nc2cc(-c3ccnc4c3cc(CN3CCC(c5ccc(C(=O)N6CCC(CN7CCN(Cc8ccc(-n9cc(N%10CCC(=O)NC%10=O)nn9)cc8)CC7)CC6)cc5)CC3)n4C)ccc2[nH]1] |
| 2c57 | 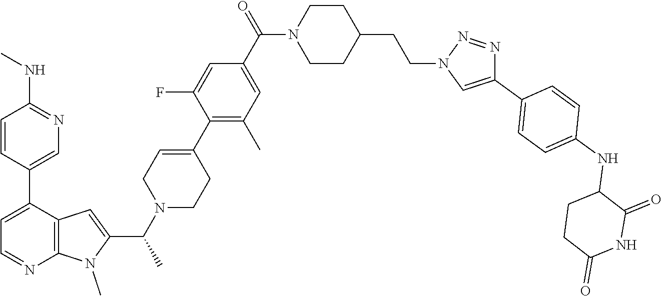 [SMILES: CNc1ccc(-c2ccnc3c2cc([C@@H](C)N2CC=C(c4c(C)cc(C(=O)N5CCC(CCn6cc(-c7ccc(NC8CCC(=O)NC8=O)cc7)nn6)CC5)cc4F)CC2)n3C)cn1] |
| 2d1 | 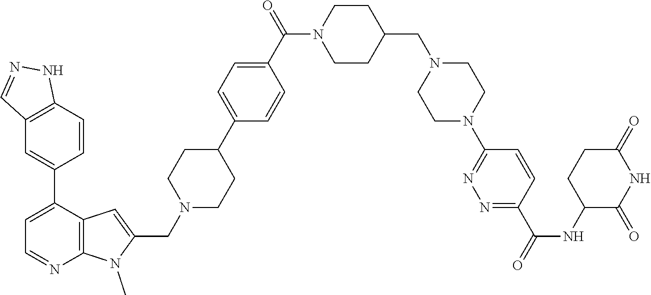 [SMILES: Cn1c(CN2CCC(c3ccc(C(=O)N4CCC(CN5CCN(c6ccc(C(=O)NC7CCC(=O)NC7=O)nn6)CC5)CC4)cc3)CC2)cc2c(-c3ccc4[nH]ncc4c3)ccnc21] |
| 2e1 | 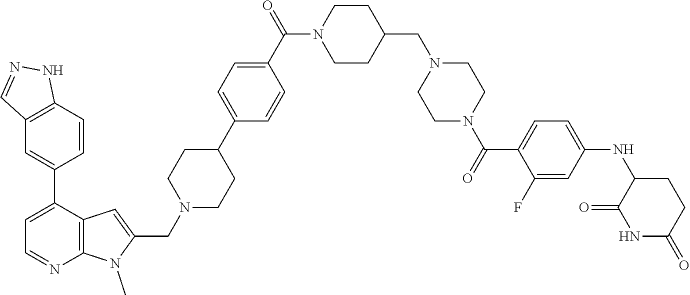 [SMILES: Cn1c(CN2CCC(c3ccc(C(=O)N4CCC(CN5CCN(C(=O)c6ccc(NC7CCC(=O)NC7=O)cc6F)CC5)CC4)cc3)CC2)cc2c(-c3ccc4[nH]ncc4c3)ccnc21] |
| 2e2 | 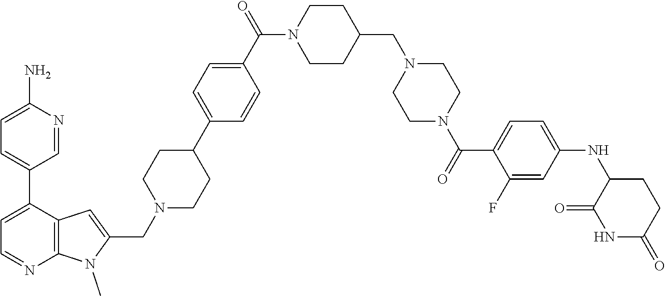 [SMILES: Cn1c(CN2CCC(c3ccc(C(=O)N4CCC(CN5CCN(C(=O)c6ccc(NC7CCC(=O)NC7=O)cc6F)CC5)CC4)cc3)CC2)cc2c(-c3ccc(N)nc3)ccnc21] |
| 2f1 | 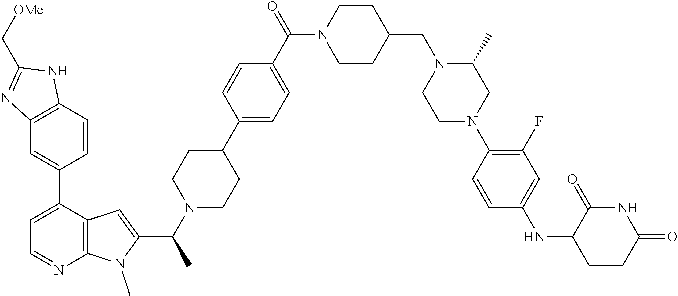 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8F)C[C@H]7C)CC6)cc5)CC3)n4C)ccc2[nH]1] |
| 2f2 | 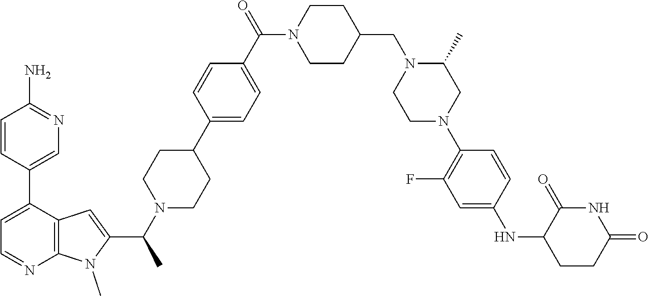 [SMILES: C[C@@H]1CN(c2ccc(NC3CCC(=O)NC3=O)cc2F)CCN1CC1CCN(C(=O)c2ccc(C3CCN([C@@H](C)c4cc5c(-c6ccc(N)nc6)ccnc5n4C)CC3)cc2)CC1] |
| 2f3 | 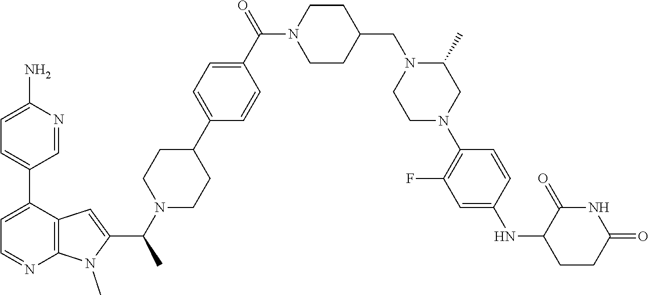 [SMILES: C[C@@H]1CN(c2ccc(NC3CCC(=O)NC3=O)cc2F)CCN1CC1CCN(C(=O)c2ccc(C3CCN([C@@H](C)c4cc5c(-c6ccc(N)nc6)ccnc5n4C)CC3)cc2)CC1] |
| 2f4 | 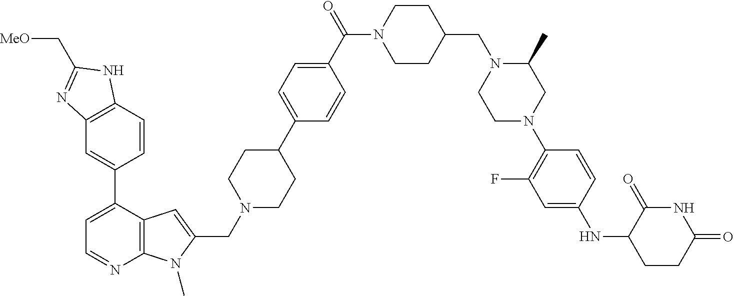 [SMILES: COCc1nc2cc(-c3ccnc4c3cc(CN3CCC(c5ccc(C(=O)N6CCC(CN7CCN(c8ccc(NC9CCC(=O)NC9=O)cc8F)C[C@@H]7C)CC6)cc5)CC3)n4C)ccc2[nH]1] |
| 2f5 | 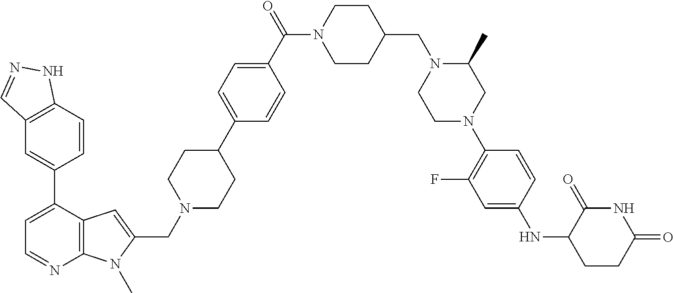 [SMILES: C[C@H]1CN(c2ccc(NC3CCC(=O)NC3=O)cc2F)CCN1CC1CCN(C(=O)c2ccc(C3CCN(Cc4cc5c(-c6ccc7[nH]ncc7c6)ccnc5n4C)CC3)cc2)CC1] |
| 2f6 | 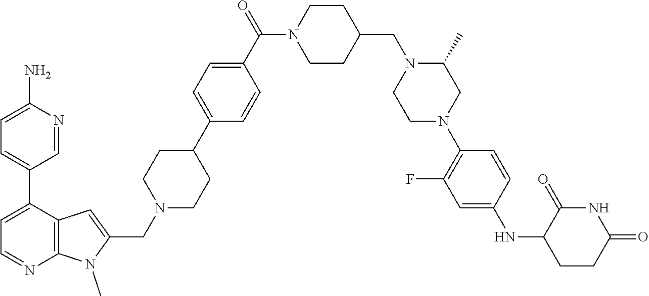 [SMILES: C[C@@H]1CN(c2ccc(NC3CCC(=O)NC3=O)cc2F)CCN1CC1CCN(C(=O)c2ccc(C3CCN(Cc4cc5c(-c6ccc(N)nc6)ccnc5n4C)CC3)cc2)CC1] |
| 2f7 | 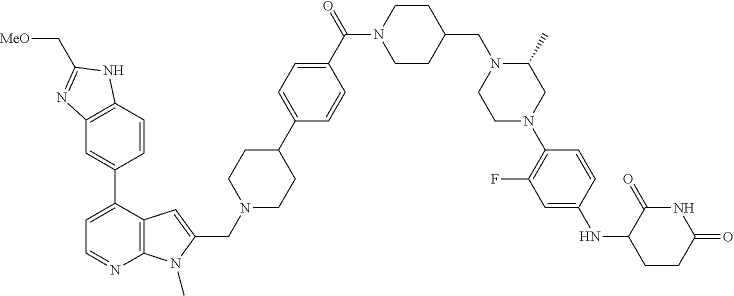 [SMILES: COCc1nc2cc(-c3ccnc4c3cc(CN3CCC(c5ccc(C(=O)N6CCC(CN7CCN(c8ccc(NC9CCC(=O)NC9=O)cc8F)C[C@H]7C)CC6)cc5)CC3)n4C)ccc2[nH]1] |
| 2f8 | 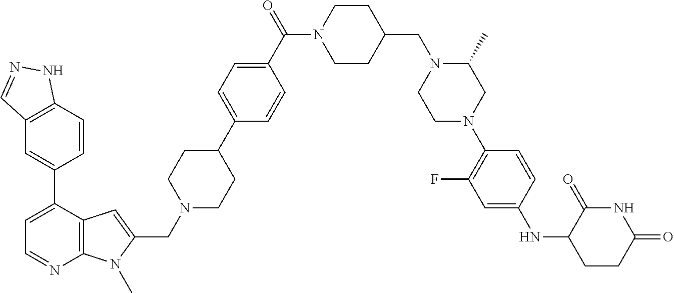 [SMILES: C[C@@H]1CN(c2ccc(NC3CCC(=O)NC3=O)cc2F)CCN1CC1CCN(C(=O)c2ccc(C3CCN(Cc4cc5c(-c6ccc7[nH]ncc7c6)ccnc5n4C)CC3)cc2)CC1] |
| 2f9 | 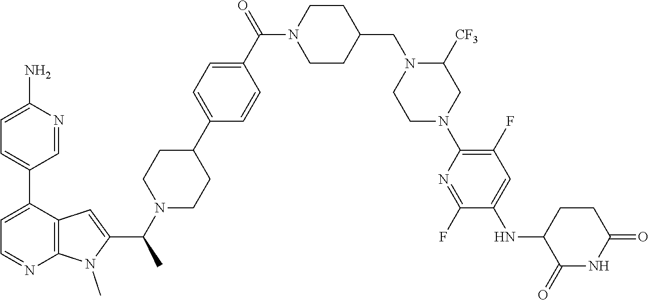 [SMILES: C[C@@H](c1cc2c(-c3ccc(N)nc3)ccnc2n1C)N1CCC(c2ccc(C(=O)N3CCC(CN4CCN(c5nc(F)c(NC6CCC(=O)NC6=O)cc5F)CC4C(F)(F)F)CC3)cc2)CC1] |
| 2f10 | 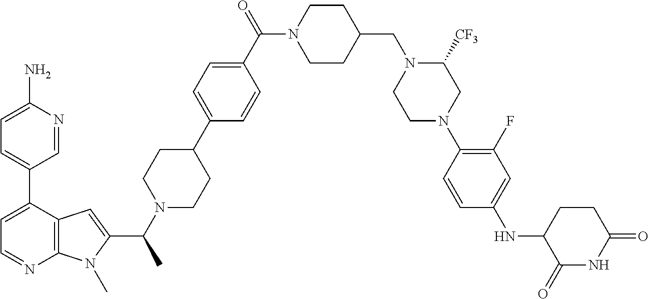 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5F)C[C@H]4C(F)(F)F)CC3)cc2)CC1] |
| 2f11 | 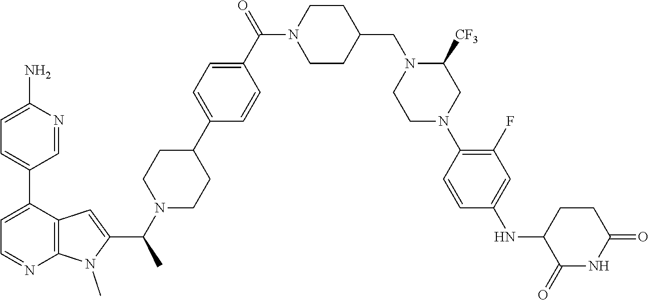 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5F)C[C@@H]4C(F)(F)F)CC3)cc2)CC1] |
| 2f12 | 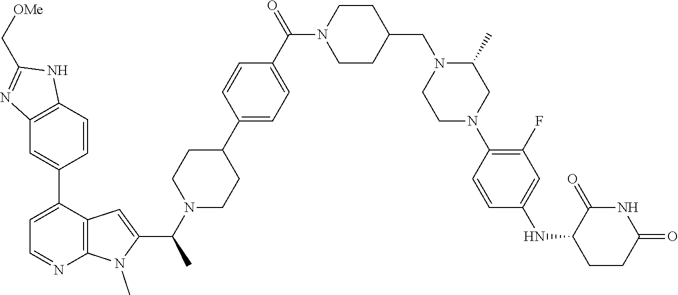 [SMILES: COCc1nc2cc(-c3ccnc4c3cc([C@H](C)N3CCC(c5ccc(C(=O)N6CCC(CN7CCN(c8ccc(N[C@H]9CCC(=O)NC9=O)cc8F)C[C@H]7C)CC6)cc5)CC3)n4C)ccc2[nH]1] |
| 2f13 | 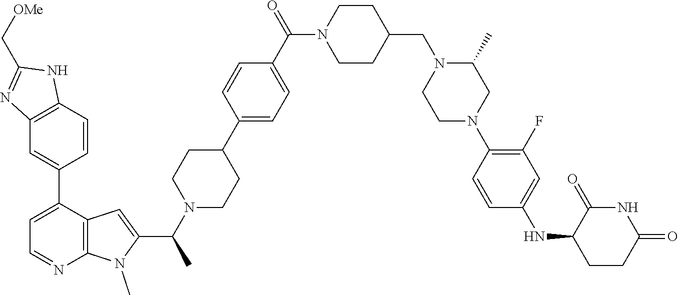 [SMILES: COCc1nc2cc(-c3ccnc4c3cc([C@H](C)N3CCC(c5ccc(C(=O)N6CCC(CN7CCN(c8ccc(N[C@@H]9CCC(=O)NC9=O)cc8F)C[C@H]7C)CC6)cc5)CC3)n4C)ccc2[nH]1] |
| 2g1 | 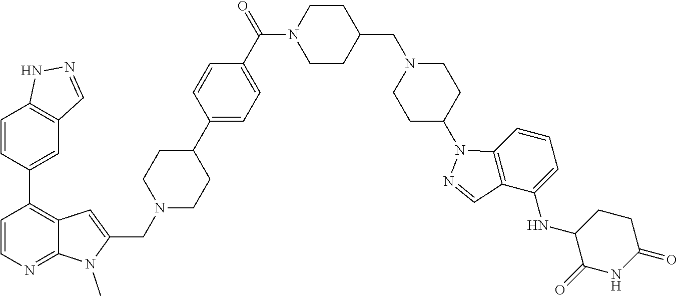 [SMILES: Cn1c(CN2CCC(c3ccc(C(=O)N4CCC(CN5CCC(n6ncc7c(NC8CCC(=O)NC8=O)cccc76)CC5)CC4)cc3)CC2)cc2c(-c3ccc4[nH]ncc4c3)ccnc21] |
| 2g63 | 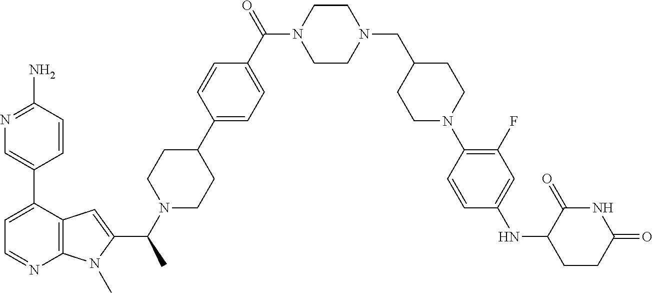 [SMILES: C[C@@H](c1cc2c(-c3ccc(N)nc3)ccnc2n1C)N1CCC(c2ccc(C(=O)N3CCN(CC4CCN(c5ccc(NC6CCC(=O)NC6=O)cc5F)CC4)CC3)cc2)CC1] |
| 2h1 | 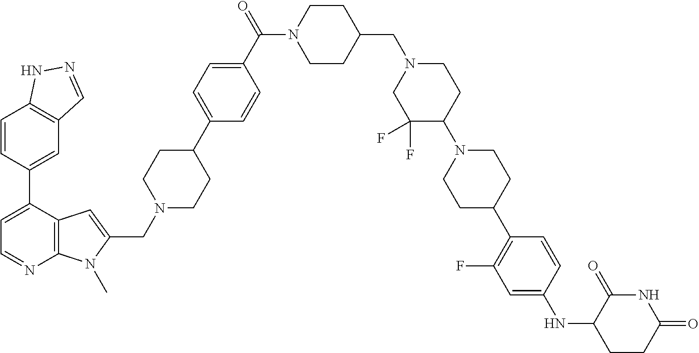 [SMILES: Cn1c(CN2CCC(c3ccc(C(=O)N4CCC(CN5CCC(N6CCC(c7ccc(NC8CCC(=O)NC8=O)cc7F)CC6)C(F)(F)C5)CC4)cc3)CC2)cc2c(-c3ccc4[nH]ncc4c3)ccnc21] |
| 2h2 | 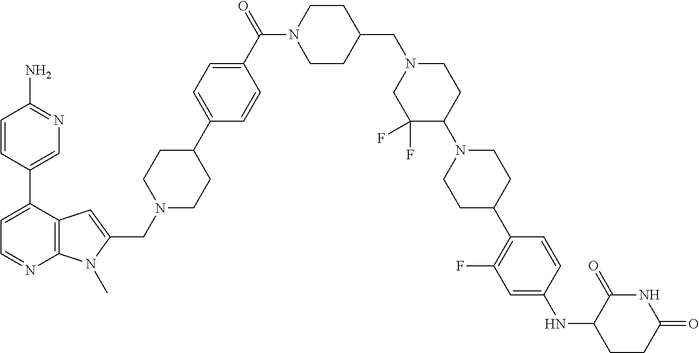 [SMILES: Cn1c(CN2CCC(c3ccc(C(=O)N4CCC(CN5CCC(N6CCC(c7ccc(NC8CCC(=O)NC8=O)cc7F)CC6)C(F)(F)C5)CC4)cc3)CC2)cc2c(-c3ccc(N)nc3)ccnc21] |
| 2h3 | 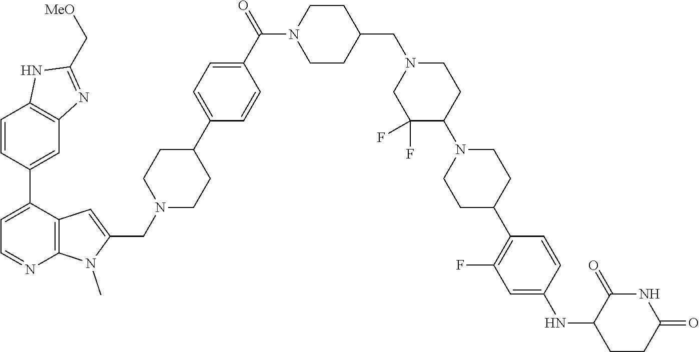 [SMILES: COCc1nc2cc(-c3ccnc4c3cc(CN3CCC(c5ccc(C(=O)N6CCC(CN7CCC(N8CCC(c9ccc(NC%10CCC(=O)NC%10=O)cc9F)CC8)C(F)(F)C7)CC6)cc5)CC3)n4C)ccc2[nH]1] |
| 2i1 | 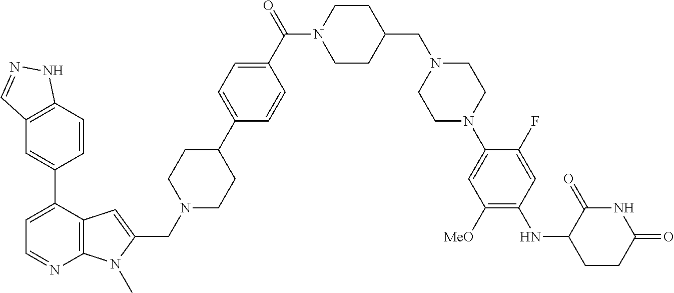 [SMILES: COc1cc(N2CCN(CC3CCN(C(=O)c4ccc(C5CCN(Cc6cc7c(-c8ccc9[nH]ncc9c8)ccnc7n6C)CC5)cc4)CC3)CC2)c(F)cc1NC1CCC(=O)NC1=O] |
| 2i2 | 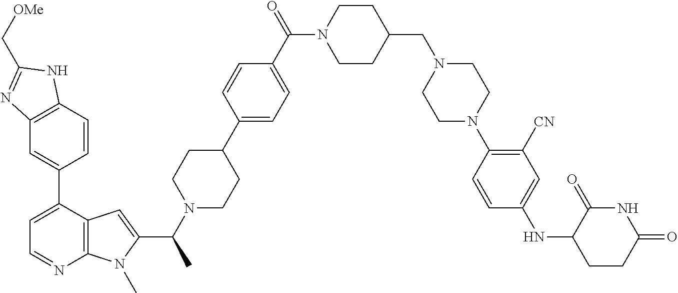 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8C#N)CC7)CC6)cc5)CC3)n4C)ccc2[nH]1] |
| 2i3 | 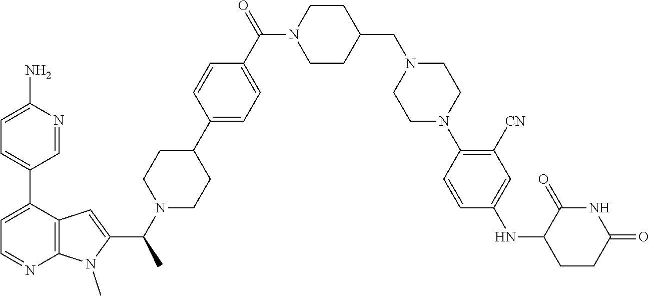 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5C#N)CC4)CC3)cc2)CC1] |
| 2i4 | 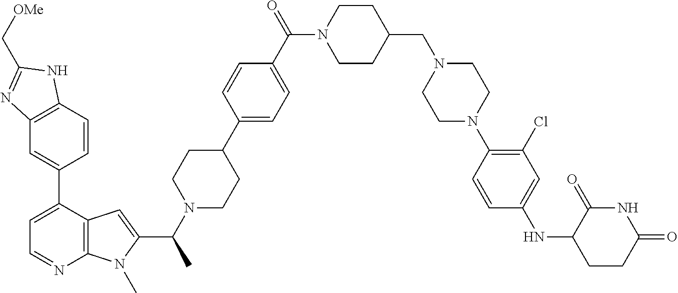 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8Cl)CC7)CC6)cc5)CC3)n4C)ccc2[nH]1] |
| 2i5 | 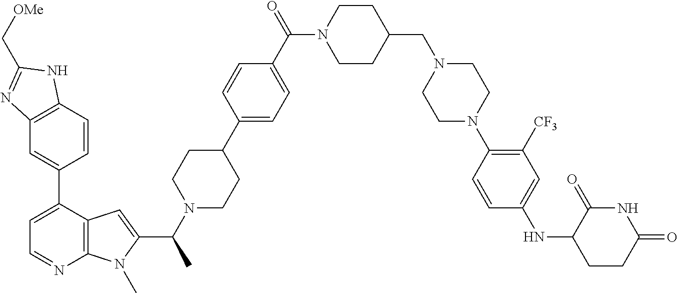 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8C(F)(F)F)CC7)CC6)cc5)CC3)n4C)ccc2[nH]1] |
| 2i6 | 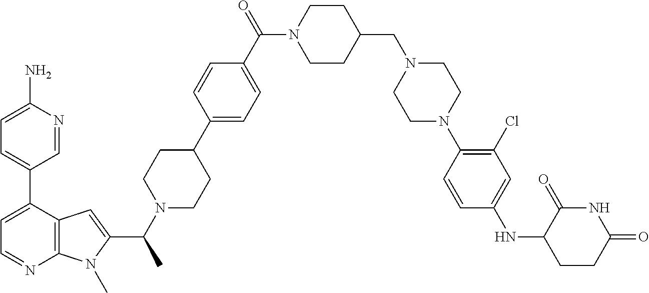 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5Cl)CC4)CC3)cc2)CC1] |
| 2i7 | 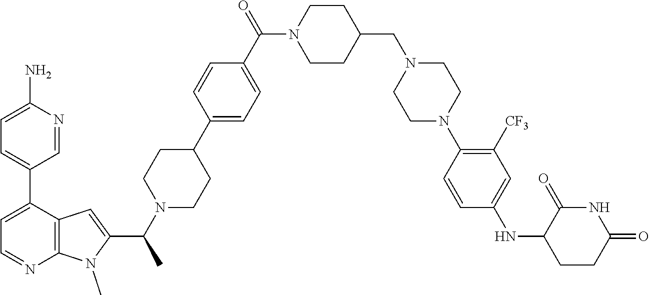 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5C(F)(F)F)CC4)CC3)cc2)CC1] |
| 2j1 | 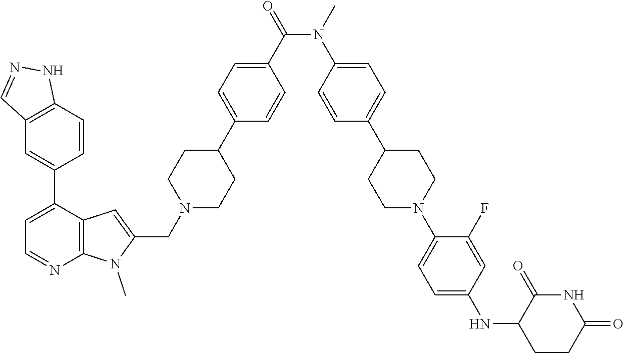 [SMILES: CN(C(=O)c1ccc(C2CCN(Cc3cc4c(-c5ccc6[nH]ncc6c5)ccnc4n3C)CC2)cc1)c1ccc(C2CCN(c3ccc(NC4CCC(=O)NC4=O)cc3F)CC2)cc1] |
| 2j2 | 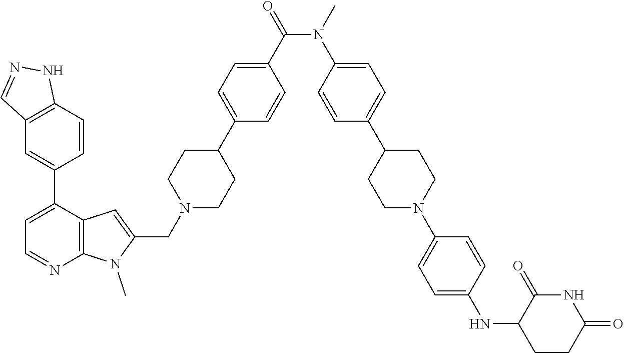 [SMILES: CN(C(=O)c1ccc(C2CCN(Cc3cc4c(-c5ccc6[nH]ncc6c5)ccnc4n3C)CC2)cc1)c1ccc(C2CCN(c3ccc(NC4CCC(=O)NC4=O)cc3)CC2)cc1] |
| 2k1 | 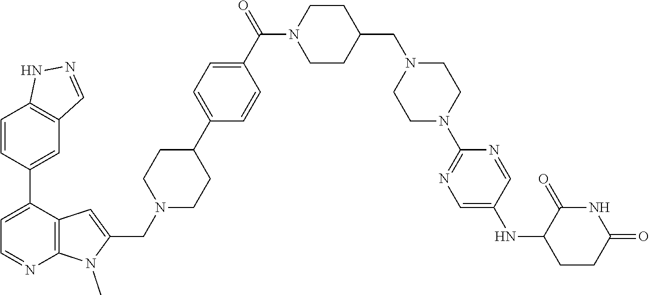 [SMILES: Cn1c(CN2CCC(c3ccc(C(=O)N4CCC(CN5CCN(c6ncc(NC7CCC(=O)NC7=O)cn6)CC5)CC4)cc3)CC2)cc2c(-c3ccc4[nH]ncc4c3)ccnc21] |
| 2k2 | 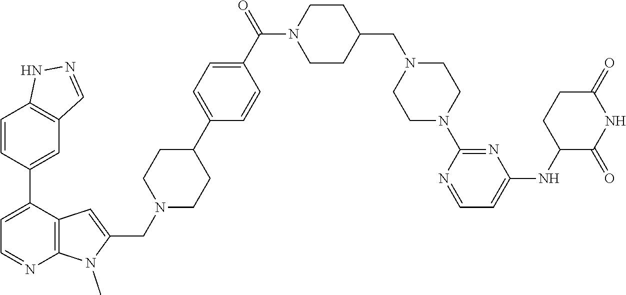 [SMILES: Cn1c(CN2CCC(c3ccc(C(=O)N4CCC(CN5CCN(c6nccc(NC7CCC(=O)NC7=O)n6)CC5)CC4)cc3)CC2)cc2c(-c3ccc4[nH]ncc4c3)ccnc21] |
| 2k3 | 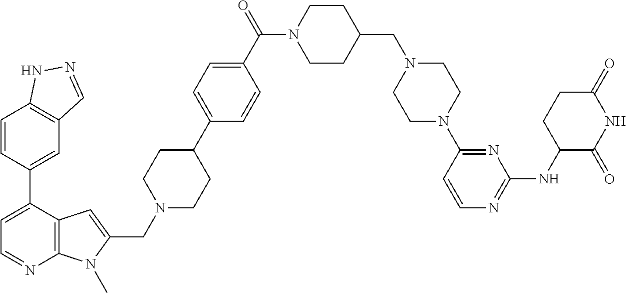 [SMILES: Cn1c(CN2CCC(c3ccc(C(=O)N4CCC(CN5CCN(c6ccnc(NC7CCC(=O)NC7=O)n6)CC5)CC4)cc3)CC2)cc2c(-c3ccc4[nH]ncc4c3)ccnc21] |
| 2l1 | 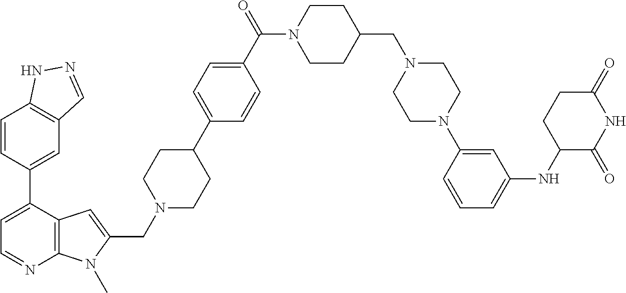 [SMILES: Cn1c(CN2CCC(c3ccc(C(=O)N4CCC(CN5CCN(c6cccc(NC7CCC(=O)NC7=O)c6)CC5)CC4)cc3)CC2)cc2c(-c3ccc4[nH]ncc4c3)ccnc21] |
| 2m1 | 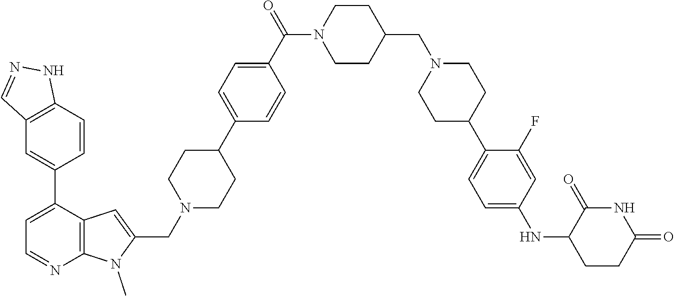 [SMILES: Cn1c(CN2CCC(c3ccc(C(=O)N4CCC(CN5CCC(c6ccc(NC7CCC(=O)NC7=O)cc6F)CC5)CC4)cc3)CC2)cc2c(-c3ccc4[nH]ncc4c3)ccnc21] |
| 2m2 | 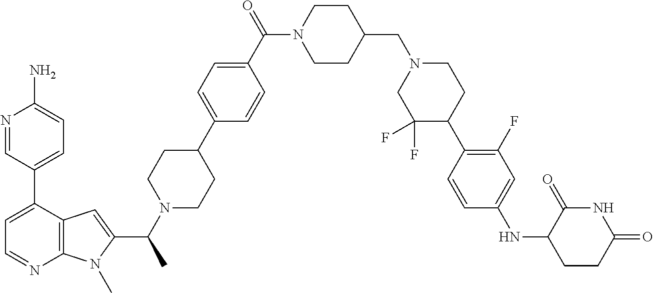 [SMILES: C[C@@H](c1cc2c(-c3ccc(N)nc3)ccnc2n1C)N1CCC(c2ccc(C(=O)N3CCC(CN4CCC(c5ccc(NC6CCC(=O)NC6=O)cc5F)C(F)(F)C4)CC3)cc2)CC1] |
| 2m3 | 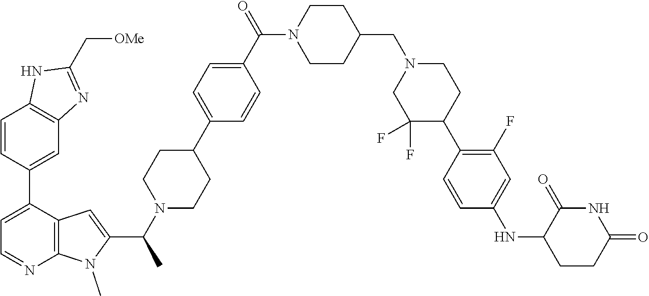 [SMILES: COCc1nc2cc(-c3ccnc4c3cc([C@H](C)N3CCC(c5ccc(C(=O)N6CCC(CN7CCC(c8ccc(NC9CCC(=O)NC9=O)cc8F)C(F)(F)C7)CC6)cc5)CC3)n4C)ccc2[nH]1] |
| 2n1 | 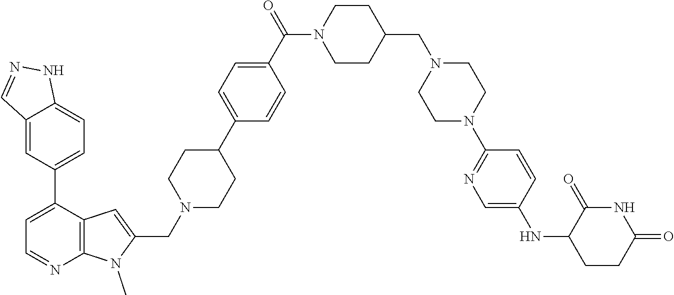 [SMILES: Cn1c(CN2CCC(c3ccc(C(=O)N4CCC(CN5CCN(c6ccc(NC7CCC(=O)NC7=O)cn6)CC5)CC4)cc3)CC2)cc2c(-c3ccc4[nH]ncc4c3)ccnc21] |
| 2n10 | 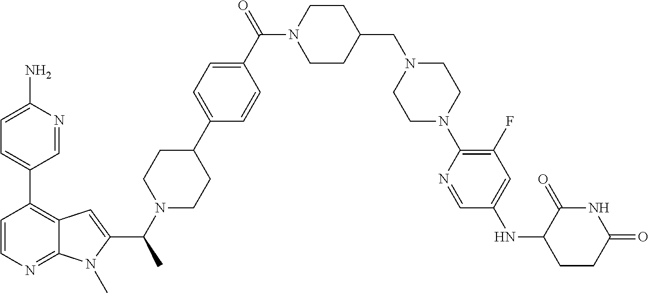 [SMILES: C[C@@H](c1cc2c(-c3ccc(N)nc3)ccnc2n1C)N1CCC(c2ccc(C(=O)N3CCC(CN4CCN(c5ncc(NC6CCC(=O)NC6=O)cc5F)CC4)CC3)cc2)CC1] |
| 2n11 | 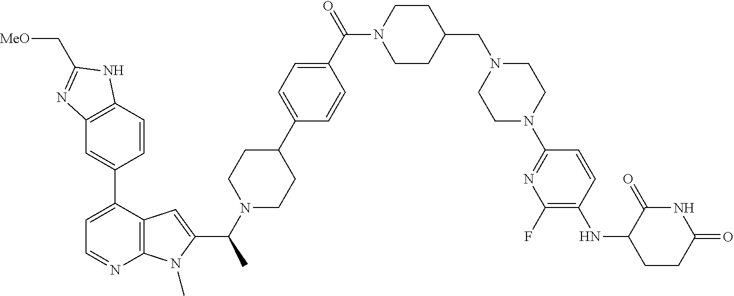 [SMILES: COCc1nc2cc(-c3ccnc4c3cc([C@H](C)N3CCC(c5ccc(C(=O)N6CCC(CN7CCN(c8ccc(NC9CCC(=O)NC9=O)c(F)n8)CC7)CC6)cc5)CC3)n4C)ccc2[nH]1] |
| 2n12 | 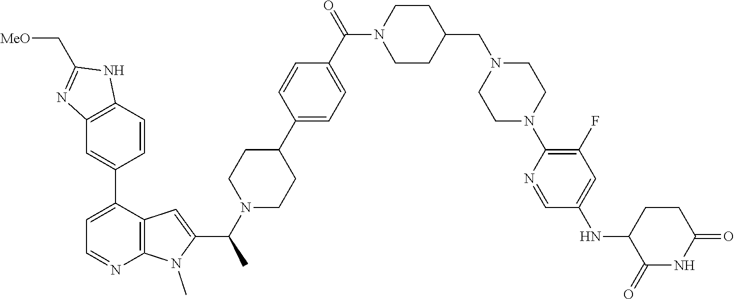 [SMILES: COCc1nc2cc(-c3ccnc4c3cc([C@H](C)N3CCC(c5ccc(C(=O)N6CCC(CN7CCN(c8ncc(NC9CCC(=O)NC9=O)cc8F)CC7)CC6)cc5)CC3)n4C)ccc2[nH]1] |
| 2n13 | 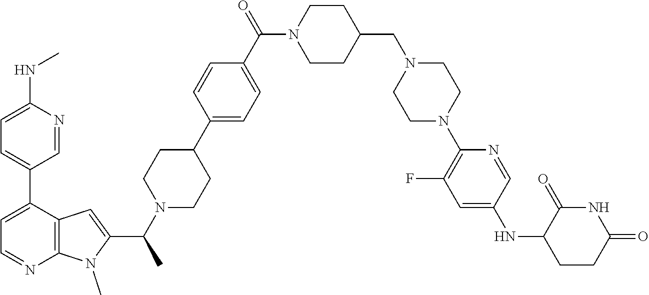 [SMILES: CNc1ccc(-c2ccnc3c2cc([C@H](C)N2CCC(c4ccc(C(=O)N5CCC(CN6CCN(c7ncc(NC8CCC(=O)NC8=O)cc7F)CC6)CC5)cc4)CC2)n3C)cn1] |
| 2n14 | 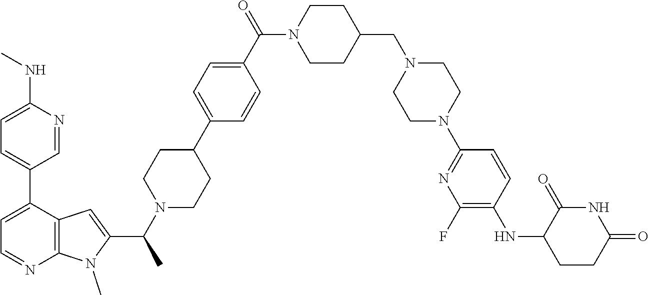 [SMILES: CNc1ccc(-c2ccnc3c2cc([C@H](C)N2CCC(c4ccc(C(=O)N5CCC(CN6CCN(c7ccc(NC8CCC(=O)NC8=O)c(F)n7)CC6)CC5)cc4)CC2)n3C)cn1] |
| 2n2 | 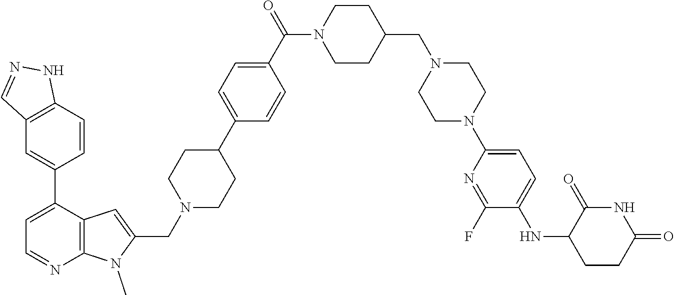 [SMILES: Cn1c(CN2CCC(c3ccc(C(=O)N4CCC(CN5CCN(c6ccc(NC7CCC(=O)NC7=O)c(F)n6)CC5)CC4)cc3)CC2)cc2c(-c3ccc4[nH]ncc4c3)ccnc21] |
| 2n3 | 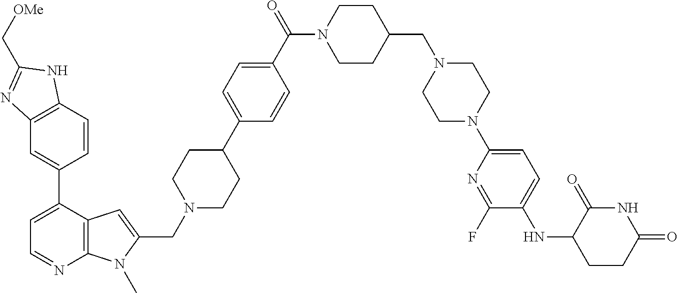 [SMILES: COCc1nc2cc(-c3ccnc4c3cc(CN3CCC(c5ccc(C(=O)N6CCC(CN7CCN(c8ccc(NC9CCC(=O)NC9=O)c(F)n8)CC7)CC6)cc5)CC3)n4C)ccc2[nH]1] |
| 2n4 | 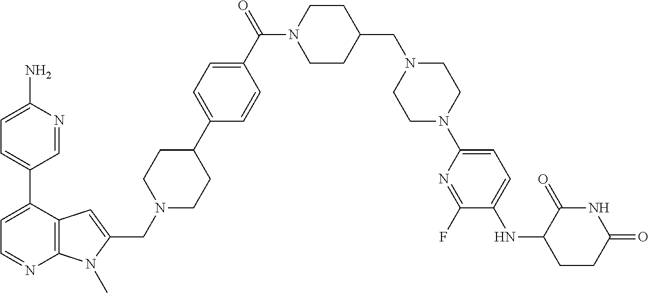 [SMILES: Cn1c(CN2CCC(c3ccc(C(=O)N4CCC(CN5CCN(c6ccc(NC7CCC(=O)NC7=O)c(F)n6)CC5)CC4)cc3)CC2)cc2c(-c3ccc(N)nc3)ccnc21] |
| 2n5 | 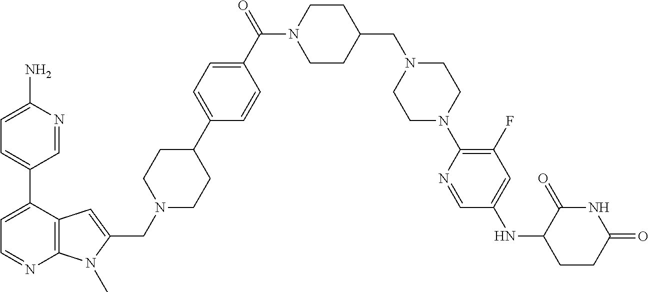 [SMILES: Cn1c(CN2CCC(c3ccc(C(=O)N4CCC(CN5CCN(c6ncc(NC7CCC(=O)NC7=O)cc6F)CC5)CC4)cc3)CC2)cc2c(-c3ccc(N)nc3)ccnc21] |
| 2n6 | 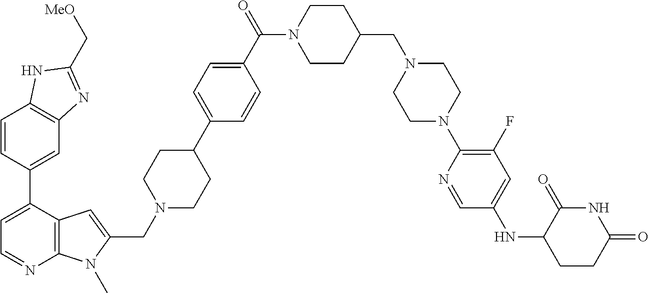 [SMILES: COCc1nc2cc(-c3ccnc4c3cc(CN3CCC(c5ccc(C(=O)N6CCC(CN7CCN(c8ncc(NC9CCC(=O)NC9=O)cc8F)CC7)CC6)cc5)CC3)n4C)ccc2[nH]1] |
| 2n7 | 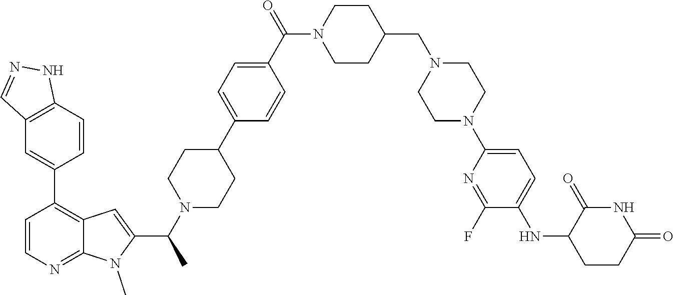 [SMILES: C[C@@H](c1cc2c(-c3ccc4[nH]ncc4c3)ccnc2n1C)N1CCC(c2ccc(C(=O)N3CCC(CN4CCN(c5ccc(NC6CCC(=O)NC6=O)c(F)n5)CC4)CC3)cc2)CC1] |
| 2n8 | 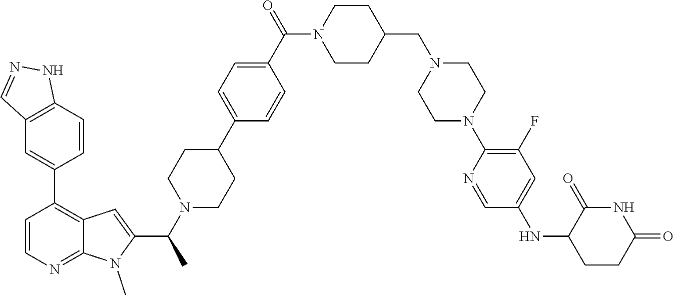 [SMILES: C[C@@H](c1cc2c(-c3ccc4[nH]ncc4c3)ccnc2n1C)N1CCC(c2ccc(C(=O)N3CCC(CN4CCN(c5ncc(NC6CCC(=O)NC6=O)cc5F)CC4)CC3)cc2)CC1] |
| 2n9 | 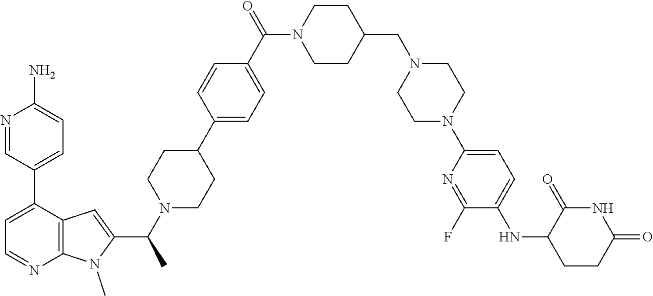 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)c(F)n5)CC4)CC3)cc2)CC1] |
| 2o1 | 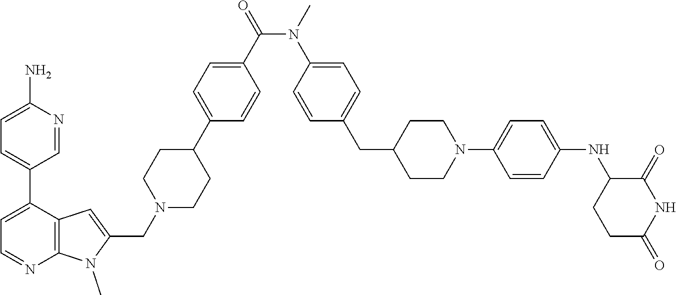 [SMILES: CN(C(=O)c1ccc(C2CCN(Cc3cc4c(-c5ccc(N)nc5)ccnc4n3C)CC2)cc1)c1ccc(CC2CCN(c3ccc(NC4CCC(=O)NC4=O)cc3)CC2)cc1] |
| 2o2 | 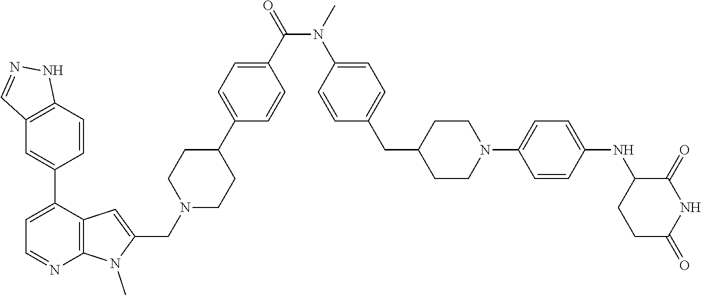 [SMILES: CN(C(=O)c1ccc(C2CCN(Cc3cc4c(-c5ccc6[nH]ncc6c5)ccnc4n3C)CC2)cc1)c1ccc(CC2CCN(c3ccc(NC4CCC(=O)NC4=O)cc3)CC2)cc1] |
| 2p1 | 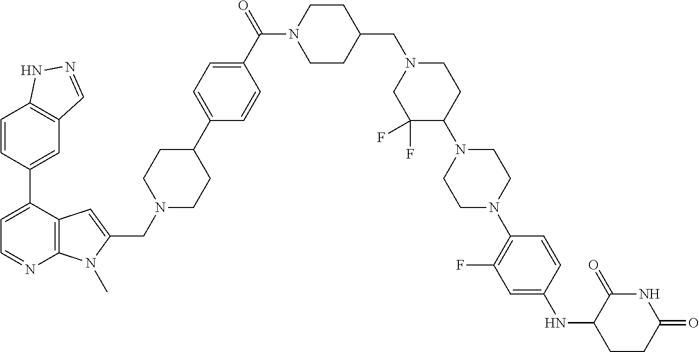 [SMILES: Cn1c(CN2CCC(c3ccc(C(=O)N4CCC(CN5CCC(N6CCN(c7ccc(NC8CCC(=O)NC8=O)cc7F)CC6)C(F)(F)C5)CC4)cc3)CC2)cc2c(-c3ccc4[nH]ncc4c3)ccnc21] |
| 2p2 | 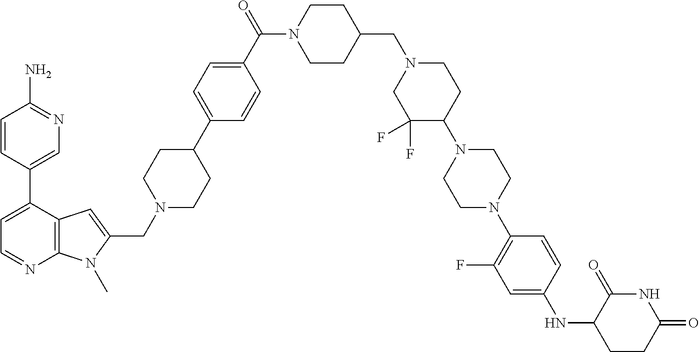 [SMILES: Cn1c(CN2CCC(c3ccc(C(=O)N4CCC(CN5CCC(N6CCN(c7ccc(NC8CCC(=O)NC8=O)cc7F)CC6)C(F)(F)C5)CC4)cc3)CC2)cc2c(-c3ccc(N)nc3)ccnc21] |
| 2p3 | 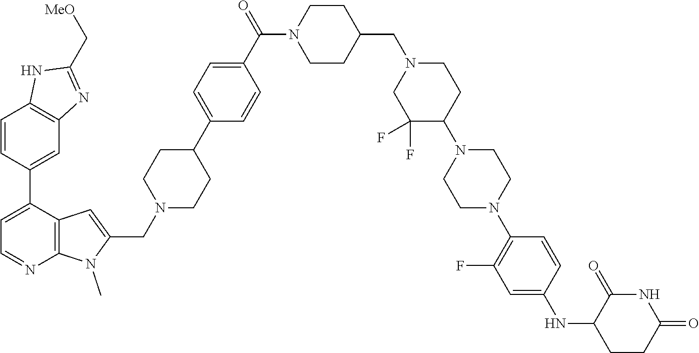 [SMILES: COCc1nc2cc(-c3ccnc4c3cc(CN3CCC(c5ccc(C(=O)N6CCC(CN7CCC(N8CCN(c9ccc(NC%10CCC(=O)NC%10=O)cc9F)CC8)C(F)(F)C7)CC6)cc5)CC3)n4C)ccc2[nH]1] |
| 2q1 | 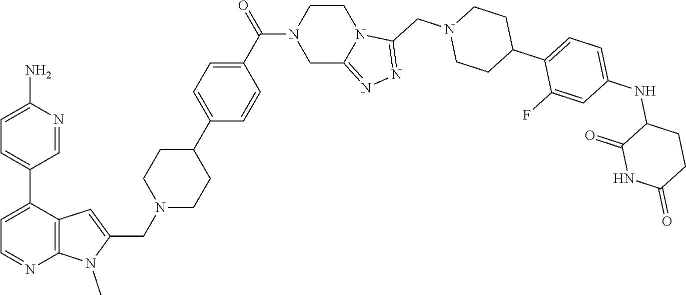 [SMILES: Cn1c(CN2CCC(c3ccc(C(=O)N4CCn5c(CN6CCC(c7ccc(NC8CCC(=O)NC8=O)cc7F)CC6)nnc5C4)cc3)CC2)cc2c(-c3ccc(N)nc3)ccnc21] |
| 2q2 | 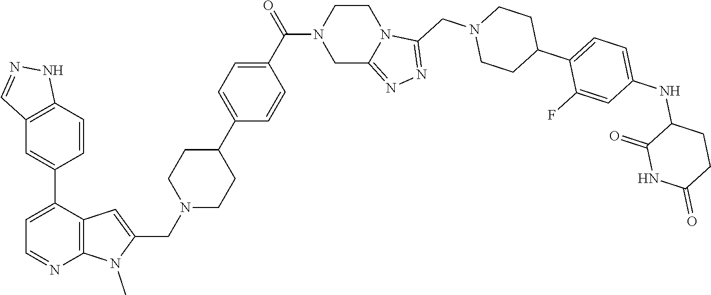 [SMILES: Cn1c(CN2CCC(c3ccc(C(=O)N4CCn5c(CN6CCC(c7ccc(NC8CCC(=O)NC8=O)cc7F)CC6)nnc5C4)cc3)CC2)cc2c(-c3ccc4[nH]ncc4c3)ccnc21] |
| 2q3 | 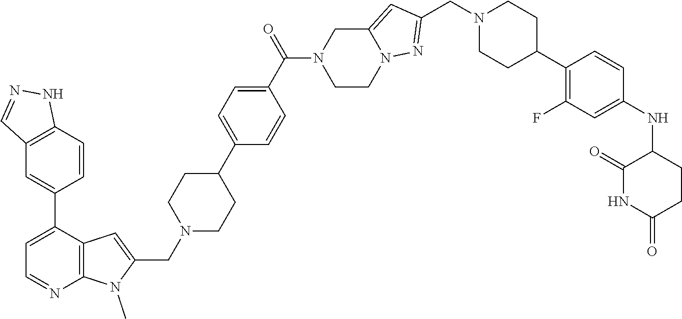 [SMILES: Cn1c(CN2CCC(c3ccc(C(=O)N4CCn5nc(CN6CCC(c7ccc(NC8CCC(=O)NC8=O)cc7F)CC6)cc5C4)cc3)CC2)cc2c(-c3ccc4[nH]ncc4c3)ccnc21] |
| 2q4 | 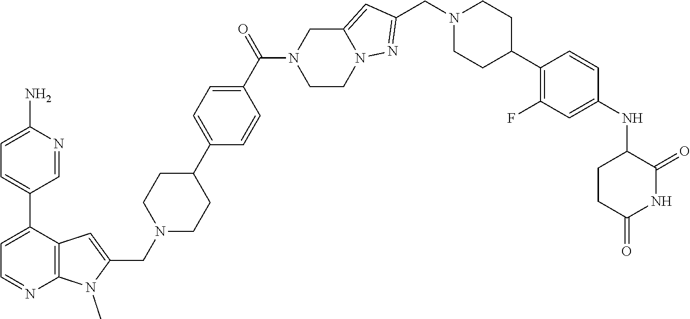 [SMILES: Cn1c(CN2CCC(c3ccc(C(=O)N4CCn5nc(CN6CCC(c7ccc(NC8CCC(=O)NC8=O)cc7F)CC6)cc5C4)cc3)CC2)cc2c(-c3ccc(N)nc3)ccnc21] |
| 2q5 | 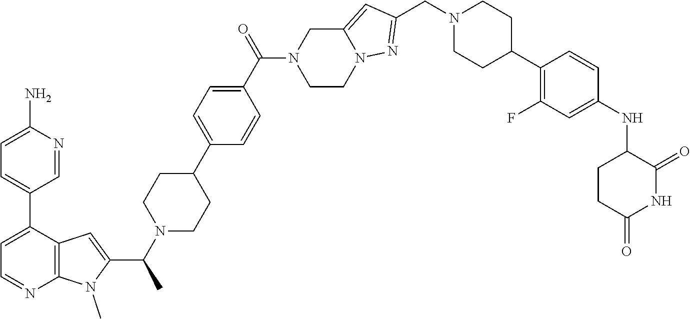 [SMILES: C[C@@H](c1cc2c(-c3ccc(N)nc3)ccnc2n1C)N1CCC(c2ccc(C(=O)N3CCn4nc(CN5CCC(c6ccc(NC7CCC(=O)NC7=O)cc6F)CC5)cc4C3)cc2)CC1] |
| 2q6 | 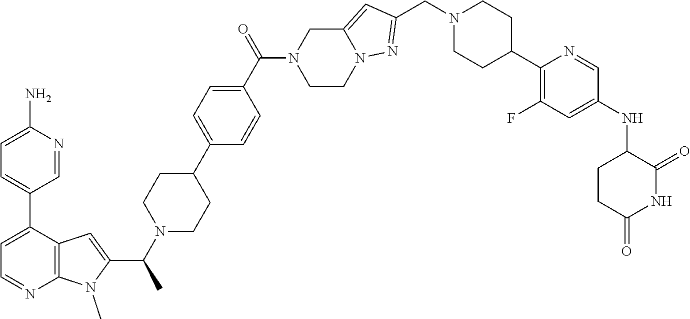 [SMILES: C[C@@H](c1cc2c(-c3ccc(N)nc3)ccnc2n1C)N1CCC(c2ccc(C(=O)N3CCn4nc(CN5CCC(c6ncc(NC7CCC(=O)NC7=O)cc6F)CC5)cc4C3)cc2)CC1] |
| 2q7 | 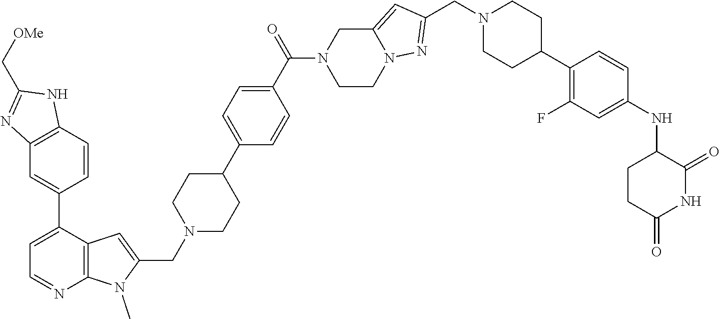 [SMILES: COCc1nc2cc(-c3ccnc4c3cc(CN3CCC(c5ccc(C(=O)N6CCn7nc(CN8CCC(c9ccc(NC%10CCC(=O)NC%10=O)cc9F)CC8)cc7C6)cc5)CC3)n4C)ccc2[nH]1] |
| 2q8 | 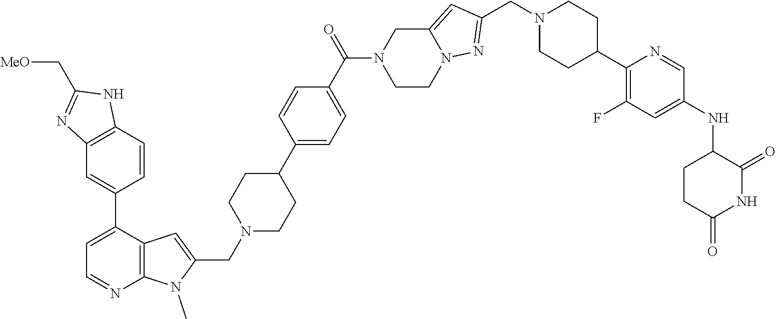 [SMILES: COCc1nc2cc(-c3ccnc4c3cc(CN3CCC(c5ccc(C(=O)N6CCn7nc(CN8CCC(c9ncc(NC%10CCC(=O)NC%10=O)cc9F)CC8)cc7C6)cc5)CC3)n4C)ccc2[nH]1] |
| 2q9 | 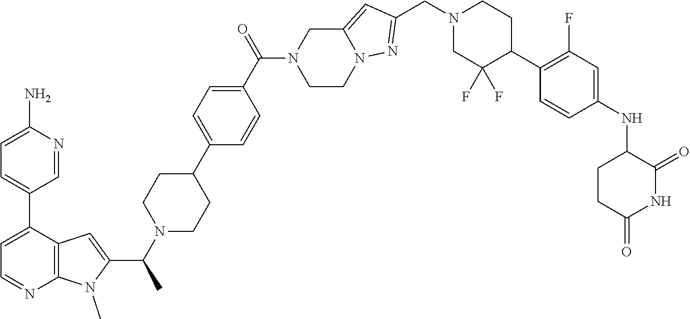 [SMILES: C[C@@H](c1cc2c(-c3ccc(N)nc3)ccnc2n1C)N1CCC(c2ccc(C(=O)N3CCn4nc(CN5CCC(c6ccc(NC7CCC(=O)NC7=O)cc6F)C(F)(F)C5)cc4C3)cc2)CC1] |
| 2q10 | 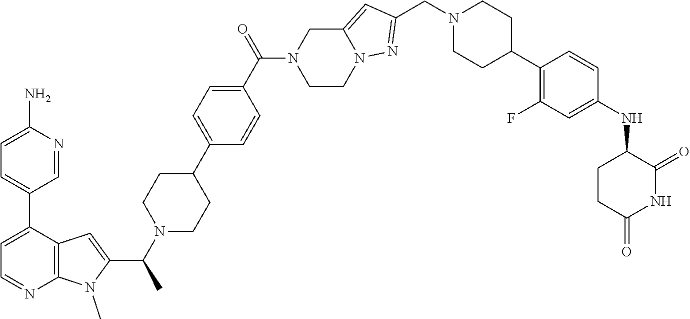 [SMILES: C[C@@H](c1cc2c(-c3ccc(N)nc3)ccnc2n1C)N1CCC(c2ccc(C(=O)N3CCn4nc(CN5CCC(c6ccc(N[C@@H]7CCC(=O)NC7=O)cc6F)CC5)cc4C3)cc2)CC1] |
| 2q11 | 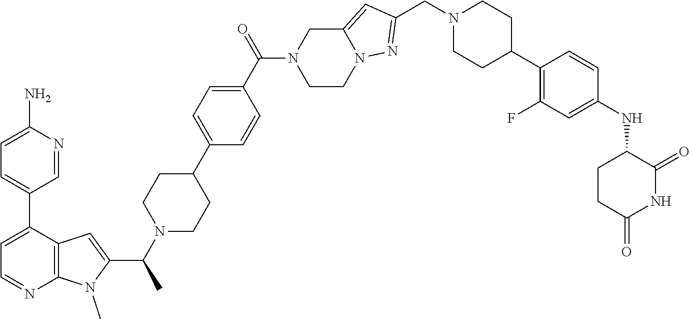 [SMILES: C[C@@H](c1cc2c(-c3ccc(N)nc3)ccnc2n1C)N1CCC(c2ccc(C(=O)N3CCn4nc(CN5CCC(c6ccc(N[C@H]7CCC(=O)NC7=O)cc6F)CC5)cc4C3)cc2)CC1] |
| 2q12 | 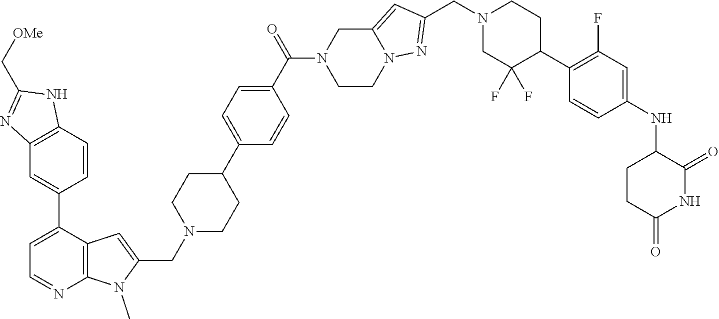 [SMILES: COCc1nc2cc(-c3ccnc4c3cc(CN3CCC(c5ccc(C(=O)N6CCn7nc(CN8CCC(c9ccc(NC%10CCC(=O)NC%10=O)cc9F)C(F)(F)C8)cc7C6)cc5)CC3)n4C)ccc2[nH]1] |
| 2q14 | 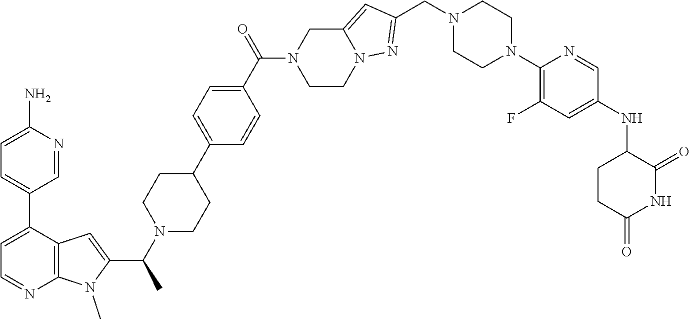 [SMILES: C[C@@H](c1cc2c(-c3ccc(N)nc3)ccnc2n1C)N1CCC(c2ccc(C(=O)N3CCn4nc(CN5CCN(c6ncc(NC7CCC(=O)NC7=O)cc6F)CC5)cc4C3)cc2)CC1] |
| 2q15 | 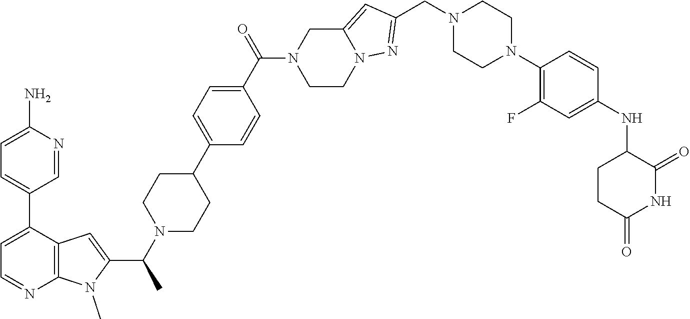 [SMILES: C[C@@H](c1cc2c(-c3ccc(N)nc3)ccnc2n1C)N1CCC(c2ccc(C(=O)N3CCn4nc(CN5CCN(c6ccc(NC7CCC(=O)NC7=O)cc6F)CC5)cc4C3)cc2)CC1] |
| 2r1 | 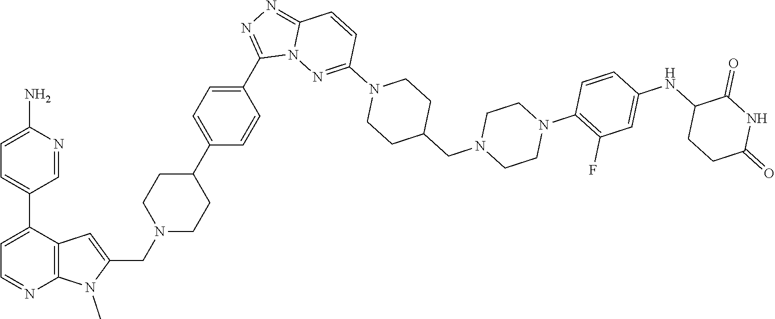 [SMILES: Cn1c(CN2CCC(c3ccc(-c4nnc5ccc(N6CCC(CN7CCN(c8ccc(NC9CCC(=O)NC9=O)cc8F)CC7)CC6)nn45)cc3)CC2)cc2c(-c3ccc(N)nc3)ccnc21] |
| 2s1 | 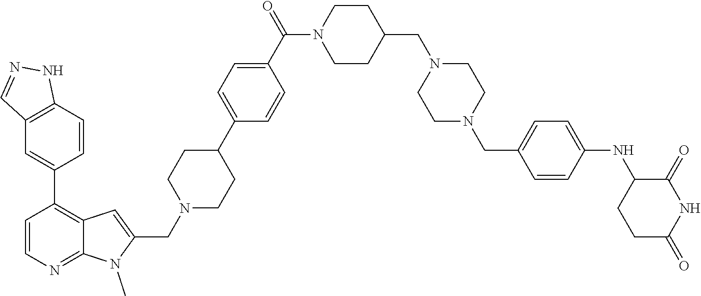 [SMILES: Cn1c(CN2CCC(c3ccc(C(=O)N4CCC(CN5CCN(Cc6ccc(NC7CCC(=O)NC7=O)cc6)CC5)CC4)cc3)CC2)cc2c(-c3ccc4[nH]ncc4c3)ccnc21] |
| 2s2 | 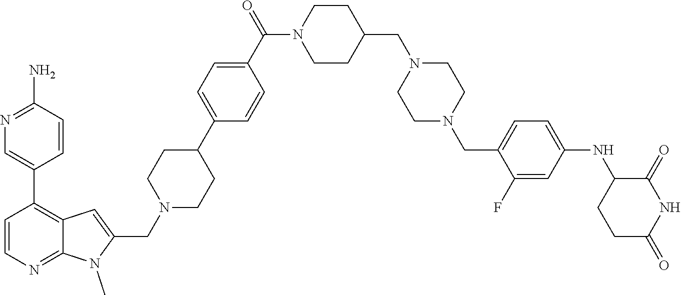 [SMILES: Cn1c(CN2CCC(c3ccc(C(=O)N4CCC(CN5CCN(Cc6ccc(NC7CCC(=O)NC7=O)cc6F)CC5)CC4)cc3)CC2)cc2c(-c3ccc(N)nc3)ccnc21] |
| 2t1 | 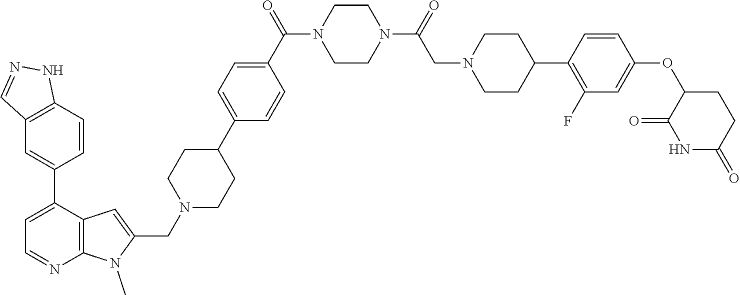 [SMILES: Cn1c(CN2CCC(c3ccc(C(=O)N4CCN(C(=O)CN5CCC(c6ccc(OC7CCC(=O)NC7=O)cc6F)CC5)CC4)cc3)CC2)cc2c(-c3ccc4[nH]ncc4c3)ccnc21] |
| 2t2 | 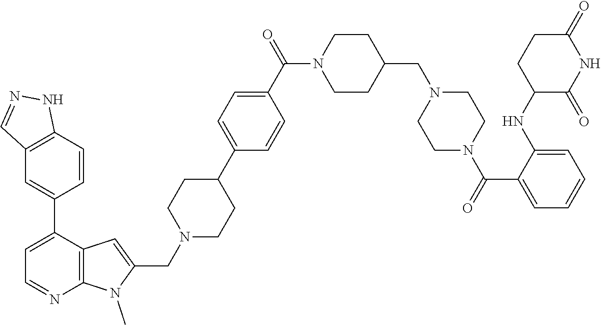 [SMILES: Cn1c(CN2CCC(c3ccc(C(=O)N4CCC(CN5CCN(C(=O)c6ccccc6NC6CCC(=O)NC6=O)CC5)CC4)cc3)CC2)cc2c(-c3ccc4[nH]ncc4c3)ccnc21] |
| 2t3 | 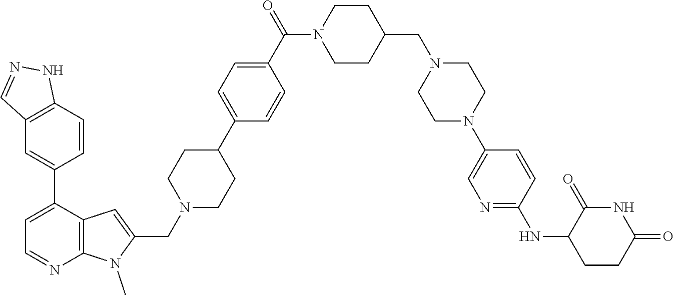 [SMILES: Cn1c(CN2CCC(c3ccc(C(=O)N4CCC(CN5CCN(c6ccc(NC7CCC(=O)NC7=O)nc6)CC5)CC4)cc3)CC2)cc2c(-c3ccc4[nH]ncc4c3)ccnc21] |
| 2t4 | 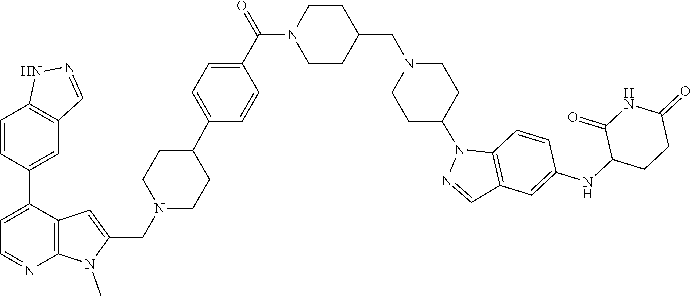 [SMILES: Cn1c(CN2CCC(c3ccc(C(=O)N4CCC(CN5CCC(n6ncc7cc(NC8CCC(=O)NC8=O)ccc76)CC5)CC4)cc3)CC2)cc2c(-c3ccc4[nH]ncc4c3)ccnc21] |
| 2t5 | 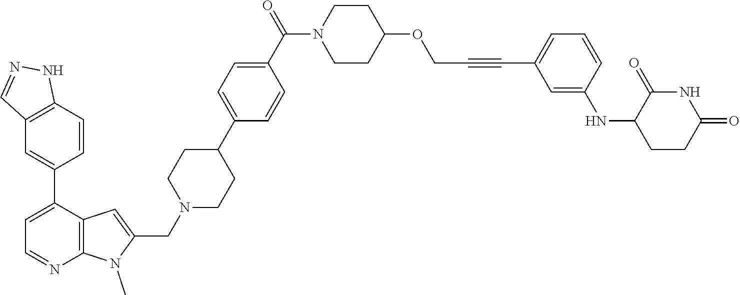 [SMILES: Cn1c(CN2CCC(c3ccc(C(=O)N4CCC(OCC#Cc5cccc(NC6CCC(=O)NC6=O)c5)CC4)cc3)CC2)cc2c(-c3ccc4[nH]ncc4c3)ccnc21] |
| 3t5 | 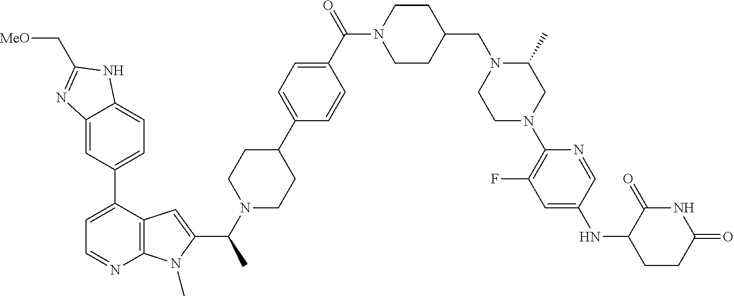 [SMILES: COCc1nc2cc(-c3ccnc4c3cc([C@H](C)N3CCC(c5ccc(C(=O)N6CCC(CN7CCN(c8ncc(NC9CCC(=O)NC9=O)cc8F)C[C@H]7C)CC6)cc5)CC3)n4C)ccc2[nH]1] |

| Example | Name |
|---|---|
| 2a1 | 3-((4-(1-(2-(4-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperazin-1-yl)-2- |
| | oxoethyl)piperidin-4-yl)-3- |
| | fluorophenyl)amino)piperidine- |
| | 2,6-dione |
| 2a2 | 3-((4-(4-(2-(4-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperazin-1-yl)-2- |
| | oxoethyl)piperazin-1-yl)-3- |
| | fluorophenyl)amino)piperidine- |
| | 2,6-dione |
| 2b1 | 3-((4-(4-((1-(4-(1-((4-(1H- |
| | indazol-5-yl)-1-methyl-1H- |
| | pyrrolo[2,3-b]pyridin-2- |
| | yl)methyl)piperidin-4- |
| | yl)benzoyl)piperidin-4- |
| | yl)methyl)piperazin-1- |
| | yl)phenyl)amino)piperidine-2,6- |
| | dione |
| 2b2 | . 3-((4-(4-((1-(4-(1-((4-(1H- |
| | indazol-5-yl)-1-methyl-1H- |
| | pyrrolo[2,3-b]pyridin-2- |
| | yl)methyl)piperidin-4- |
| | yl)benzoyl)piperidin-4- |
| | yl)methyl)piperazin-1-yl)-2- |
| | fluorophenyl)amino)piperidine- |
| | 2,6-dione |
| 2b3 | 3-((2-fluoro-4-(4-((1-(4-(1-((4- |
| | (2-(methoxymethyl)-1H- |
| | benzo[d]imidazol-5-yl)-1- |
| | methyl-1H-pyrrolo[2,3- |
| | b]pyridin-2-yl)methyl)piperidin- |
| | 4-yl)benzoyl)piperidin-4- |
| | yl)methyl)piperazin-1- |
| | yl)phenyl)amino)piperidine-2,6- |
| | dione |
| 2c1 | 3-((4-(4-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperazin-1-yl)-3- |
| | fluorophenyl)amino)piperidine- |
| | 2,6-dione |
| 2c2 | 3-((4-(4-((1-(4-(1-((4-(6- |
| | aminopyridin-3-yl)-1-methyl- |
| | 1H-pyrrolo[2,3-b]pyridin-2- |
| | yl)methyl)-piperidin-4- |
| | yl)benzoyl)piperidin-4- |
| | yl)methyl)piperazin-1-yl)-3- |
| | fluorophenyl)amino)piperidine- |
| | 2,6-dione |
| 2c3 | 3-((4-(4-((1-(4-(1-((4-(6-amino- |
| | 5-fluoropyridin-3-yl)-1-methyl- |
| | 1H-pyrrolo[2,3-b]pyridin-2-yl)- |
| | methyl)piperidin-4- |
| | yl)benzoyl)piperidin-4- |
| | yl)methyl)piperazin-1-yl)-3- |
| | fluorophenyl)amino)piperidine- |
| | 2,6-dione |
| 2c4 | 3-((3-fluoro-4-(4-((1-(4-(1-((4- |
| | (2-(methoxymethyl)-1H- |
| | benzo[d]imidazol-5-yl)-1- |
| | methyl-1H-pyrrolo[2,3- |
| | b]pyridin-2-yl)methyl)piperidin- |
| | 4-yl)benzoyl)piperidin-4- |
| | yl)methyl)piperazin-1- |
| | yl)phenyl)-amino)piperidine-2,6- |
| | dione |
| 2c5 | (S)-3-((4-(4-((1-(4-(1-((4-(1H- |
| | indazol-5-yl)-1-methyl-1H- |
| | pyrrolo[2,3-b]pyridin-2- |
| | yl)methyl)piperidin-4- |
| | yl)benzoyl)piperidin-4-yl)- |
| | methyl)piperazin-1-yl)-3- |
| | fluorophenyl)amino)piperidine- |
| | 2,6-dione |
| 2c6 | (R)-3-((4-(4-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperazin-1-yl)-3- |
| | fluorophenyl)amino)piperidine- |
| | 2,6-dione |
| 2c7 | 3-((4-(4-((1-(4-(1-((4-(6-amino- |
| | 2-methylpyridin-3-yl)-1-methyl- |
| | 1H-pyrrolo[2,3-b]pyridin-2- |
| | yl)methyl)piperidin-4- |
| | yl)benzoyl)piperidin-4- |
| | yl)methyl)piperazin-1-yl)-3- |
| | fluorophenyl)amino)piperidine- |
| | 2,6-dione |
| 2c8 | 6-amino-3-(2-((4-(4-(4-((4-(4- |
| | ((2,6-dioxopiperidin-3- |
| | yl)amino)-2- |
| | fluorophenyl)piperazin-1-yl)- |
| | methyl)piperidine-1- |
| | carbonyl)phenyl)piperidin-1- |
| | yl)methyl)-1-methyl-1H- |
| | pyrrolo[2,3-b]pyridin-4-yl)- |
| | picolinonitrile |
| 2c9 | 3-((4-(4-((1-(4-(1-((4-(6- |
| | aminopyridin-3-yl)-1-methyl- |
| | 1H-pyrrolo[2,3-b]pyridin-2- |
| | yl)methyl)-1,2,3,6- |
| | tetrahydropyridin-4-yl)-3- |
| | methylbenzoyl)piperidin-4- |
| | yl)methyl)piperazin-1-yl)-3- |
| | fluorophenyl)-amino)piperidine- |
| | 2,6-dione |
| 2c10 | 3-((4-(4-((1-(4-(1-(1-(4-(6- |
| | aminopyridin-3-yl)-1-methyl- |
| | 1H-pyrrolo[2,3-b]pyridin-2-yl)- |
| | ethyl)-piperidin-4- |
| | yl)benzoyl)piperidin-4- |
| | yl)methyl)piperazin-1-yl)-3- |
| | fluorophenyl)amino)piperidine- |
| | 2,6-dione |
| 2c11 | (3R)-3-((4-(4-((1-(4-(1-(1-(4-(6- |
| | aminopyridin-3-yl)-1-methyl- |
| | 1H-pyrrolo[2,3-b]pyridin-2- |
| | yl)ethyl)piperidin-4- |
| | yl)benzoyl)piperidin-4- |
| | yl)methyl)piperazin-1-yl)-3- |
| | fluorophenyl)amino)piperidine- |
| | 2,6-dione |
| 2c12 | (3S)-3-((4-(4-((1-(4-(1-(1-(4-(6- |
| | aminopyridin-3-yl)-1-methyl- |
| | 1H-pyrrolo[2,3-b]pyridin-2-yl)- |
| | ethyl)piperidin-4- |
| | yl)benzoyl)piperidin-4- |
| | yl)methyl)piperazin-1-yl)-3- |
| | fluorophenyl)amino)piperidine- |
| | 2,6-dione |
| 2c13 | 3-((4-(4-((1-(4-(1-((R)-1-(4-(6- |
| | aminopyridin-3-yl)-1-methyl- |
| | 1H-pyrrolo[2,3-b]pyridin-2- |
| | yl)ethyl)-piperidin-4- |
| | yl)benzoyl)piperidin-4- |
| | yl)methyl)piperazin-1-yl)-3- |
| | fluorophenyl)amino)piperidine- |
| | 2,6-dione |
| 2c14 | 3-((4-(4-((1-(4-(1-((S)-1-(4-(6- |
| | aminopyridin-3-yl)-1-methyl- |
| | 1H-pyrrolo[2,3-b]pyridin-2- |
| | yl)ethyl)-piperidin-4- |
| | yl)benzoyl)piperidin-4- |
| | yl)methyl)piperazin-1-yl)-3- |
| | fluorophenyl)amino)piperidine- |
| | 2,6-dione |
| 2c15 | 3-((4-(4-((1-(4-(1-((4-(6- |
| | aminopyridin-3-yl)-1-methyl- |
| | 1H-pyrrolo[2,3-b]pyridin-2- |
| | yl)methyl)-1,2,3,6- |
| | tetrahydropyridin-4-yl)-3,5- |
| | dimethylbenzoyl)piperidin-4- |
| | yl)methyl)piperazin-1-yl)-3- |
| | fluorophenyl)amino)piperidine- |
| | 2,6-dione |
| 2c16 | 3-((3-fluoro-4-(4-((1-(4-(1-((S)- |
| | 1-(4-(2-(methoxymethyl)-1H- |
| | benzo[d]imidazol-6-yl)-1- |
| | methyl-1H-pyrrolo[2,3- |
| | b]pyridin-2-yl)ethyl)piperidin-4- |
| | yl)benzoyl)piperidin-4- |
| | yl)methyl)piperazin-1- |
| | yl)phenyl)amino)piperidine-2,6- |
| | dione |
| 2c17 | 3-((4-(4-((1-(4-(1-((S)-1-(4-(1H- |
| | indazol-5-yl)-1-methyl-1H- |
| | pyrrolo[2,3-b]pyridin-2-yl)- |
| | ethyl)piperidin-4- |
| | yl)benzoyl)piperidin-4- |
| | yl)methyl)piperazin-1-yl)-3- |
| | fluorophenyl)amino)piperidine- |
| | 2,6-dione |
| 2c18 | 3-((3-fluoro-4-(4-((1-(4-(1-((S)- |
| | 1-(4-(2-((S)-1-methoxyethyl)- |
| | 1H-benzo[d]imidazol-5-yl)-1- |
| | methyl-1H-pyrrolo[2,3- |
| | b]pyridin-2-yl)ethyl)piperidin-4- |
| | yl)benzoyl)piperidin-4- |
| | yl)methyl)piperazin-1- |
| | yl)phenyl)amino)piperidine-2,6- |
| | dione |
| 2c19 | (S)-3-((4-(4-((1-(4-(1-((S)-1-(4- |
| | (6-aminopyridin-3-yl)-1-methyl- |
| | 1H-pyrrolo[2,3-b]pyridin-2- |
| | yl)ethyl)piperidin-4- |
| | yl)benzoyl)piperidin-4- |
| | yl)methyl)piperazin-1-yl)-3- |
| | fluorophenyl)-amino)- |
| | piperidine-2,6-dione |
| 2c20 | (R)-3-((4-(4-((1-(4-(1-((S)-1-(4- |
| | (6-aminopyridin-3-yl)-1-methyl- |
| | 1H-pyrrolo[2,3-b]pyridin-2- |
| | yl)ethyl)-piperidin-4- |
| | yl)benzoyl)piperidin-4- |
| | yl)methyl)piperazin-1-yl)-3- |
| | fluorophenyl)amino)-piperidine- |
| | 2,6-dione |
| 2c21 | 3-((3-fluoro-4-(4-((1-(4-(1-((S)- |
| | 1-(4-(2-((R)-1-methoxyethyl)- |
| | 1H-benzo[d]imidazol-5-yl)-1- |
| | methyl-1H-pyrrolo[2,3- |
| | b]pyridin-2-yl)ethyl)piperidin-4- |
| | yl)benzoyl)piperidin-4- |
| | yl)methyl)piperazin-1- |
| | yl)phenyl)amino)piperidine-2,6- |
| | dione |
| 2c22 | 6-amino-3-(2-((1S)-1-(4-(4-(4- |
| | ((4-(4-((2,6-dioxopiperidin-3- |
| | yl)amino)-2-fluorophenyl)- |
| | piperazin-1- |
| | yl)methyl)piperidine-1- |
| | carbonyl)phenyl)piperidin-1- |
| | yl)ethyl)-1-methyl-1H- |
| | pyrrolo[2,3-b]pyridin-4- |
| | yl)picolinonitrile |
| 2c23 | 3-((4-(4-((1-(4-(1-((S)-1-(4-(6- |
| | aminopyridin-3-yl)-1-methyl- |
| | 1H-pyrrolo[2,3-b]pyridin-2- |
| | yl)ethyl)-piperidin-4- |
| | yl)benzoyl)-4-fluoropiperidin-4- |
| | yl)methyl)piperazin-1-yl)-3- |
| | fluorophenyl)amino)-piperidine- |
| | 2,6-dione |
| 2c24 | 3-((4-(4-((1-(4-(1-((S)-1-(4-(6- |
| | amino-5-fluoropyridin-3-yl)-1- |
| | methyl-1H-pyrrolo[2,3- |
| | b]pyridin-2-yl)ethyl)piperidin-4- |
| | yl)benzoyl)piperidin-4- |
| | yl)methyl)piperazin-1-yl)-3- |
| | fluorophenyl)-amino)piperidine- |
| | 2,6-dione |
| 2c26 | 3-((3-fluoro-4-(4-((1-(4-(1-((S)- |
| | 1-(4-(6-((2- |
| | hydroxyethyl)amino)pyridin-3- |
| | yl)-1-methyl-1H-pyrrolo[2,3- |
| | b]pyridin-2-yl)ethyl)piperidin-4- |
| | yl)benzoyl)piperidin-4- |
| | yl)methyl)piperazin-1- |
| | yl)phenyl)-amino)piperidine-2,6- |
| | dione |
| 2c27 | 3-((4-(4-((1-(4-(1-((S)-1-(4-(6- |
| | aminopyridazin-3-yl)-1-methyl- |
| | 1H-pyrrolo[2,3-b]pyridin-2- |
| | yl)ethyl)piperidin-4- |
| | yl)benzoyl)piperidin-4- |
| | yl)methyl)piperazin-1-yl)-3- |
| | fluorophenyl)amino)piperidine- |
| | 2,6-dione |
| 2c28 | 3-((3-fluoro-4-(4-((1-(4-(1-((S)- |
| | 1-(1-methyl-4-(3-methyl-2-oxo- |
| | 2,3-dihydro-1H- |
| | benzo[d]imidazol-5-yl)-1H- |
| | pyrrolo[2,3-b]pyridin-2- |
| | yl)ethyl)piperidin-4- |
| | yl)benzoyl)piperidin-4- |
| | yl)methyl)piperazin-1- |
| | yl)phenyl)amino)piperidine-2,6- |
| | dione |
| 2c30 | 3-((4-(4-((1-(4-(1-((S)-1-(4-(5- |
| | amino-3-fluoropyridin-2-yl)-1- |
| | methyl-1H-pyrrolo[2,3- |
| | b]pyridin-2-yl)ethyl)piperidin-4- |
| | yl)benzoyl)piperidin-4- |
| | yl)methyl)piperazin-1-yl)-3- |
| | fluorophenyl)-amino)piperidine- |
| | 2,6-dione |
| 2c32 | 3-((4-(4-((1-(4-(1-((S)-1-(4-(6- |
| | amino-2-methoxypyridin-3-yl)- |
| | 1-methyl-1H-pyrrolo[2,3- |
| | b]pyridin-2-yl)ethyl)piperidin-4- |
| | yl)benzoyl)piperidin-4- |
| | yl)methyl)piperazin-1-yl)-3- |
| | fluorophenyl)amino)-piperidine- |
| | 2,6-dione |
| 2c33 | 3-((4-(4-((1-(4-(1-((S)-1-(4-(5- |
| | aminopyridin-2-yl)-1-methyl- |
| | 1H-pyrrolo[2,3-b]pyridin-2- |
| | yl)ethyl)-piperidin-4- |
| | yl)benzoyl)piperidin-4- |
| | yl)methyl)piperazin-1-yl)-3- |
| | fluorophenyl)amino)piperidine- |
| | 2,6-dione |
| 2c34 | 3-((4-(4-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperazin-1-yl)-3,5- |
| | difluorophenyl)amino)piperidine- |
| | 2,6-dione |
| 2c37 | 3-((4-(1-((5-(1′-((4-(6- |
| | aminopyridin-3-yl)-1-methyl- |
| | 1H-pyrrolo[2,3-b]pyridin-2- |
| | yl)methyl)-1′,2′,3′,6′-tetrahydro- |
| | [2,4′-bipyridine]-5-carbonyl)- |
| | 4,5,6,7-tetrahydropyrazolo[1,5- |
| | a]pyrazin-2-yl)methyl)piperidin- |
| | 4-yl)-3- |
| | fluorophenyl)amino)piperidine- |
| | 2,6-dione |
| 2c38 | 3-((4-(1-((5-(1′-((4-(6- |
| | aminopyridazin-3-yl)-1-methyl- |
| | 1H-pyrrolo[2,3-b]pyridin-2- |
| | yl)methyl)-1′,2′,3′,6′-tetrahydro- |
| | [2,4′-bipyridine]-5-carbonyl)- |
| | 4,5,6,7-tetrahydropyrazolo[1,5- |
| | a]pyrazin-2-yl)methyl)piperidin- |
| | 4-yl)-3- |
| | fluorophenyl)amino)piperidine- |
| | 2,6-dione |
| 2c39 | 3-((4-(4-((1-(4-(4-((4-(6- |
| | aminopyridin-3-yl)-1-methyl- |
| | 1H-pyrrolo[2,3-b]pyridin-2- |
| | yl)methyl)piperazin-1- |
| | yl)benzoyl)piperidin-4- |
| | yl)methyl)piperazin-1-yl)-3- |
| | fluorophenyl)amino)piperidine- |
| | 2,6-dione |
| 2c40 | 3-((4-(4-((1-(4-(1-((S)-1-(4-(6- |
| | aminopyridin-3-yl)-1-methyl- |
| | 1H-pyrrolo[2,3-b]pyridin-2- |
| | yl)ethyl)piperidin-4- |
| | yl)benzoyl)piperidin-4- |
| | yl)methyl)piperazin-1-yl)-2- |
| | methoxyphenyl)amino)piperidine- |
| | 2,6-dione |
| 2c41 | 3-((3-fluoro-4-(1-((5-(4-(1-((S)- |
| | 1-(1-methyl-4-(6- |
| | (methylamino)pyridin-3-yl)-1H- |
| | pyrrolo[2,3-b]pyridin-2- |
| | yl)ethyl)piperidin-4-yl)benzoyl)- |
| | 4,5,6,7-tetrahydropyrazolo[1,5- |
| | a]pyrazin-2-yl)methyl)piperidin- |
| | 4-yl)phenyl)amino)piperidine- |
| | 2,6-dione |
| 2c42 | (S)-3-((3-fluoro-4-(1-((5-(4-(1- |
| | ((S)-1-(1-methyl-4-(6- |
| | (methylamino)pyridin-3-yl)-1H- |
| | pyrrolo[2,3-b]pyridin-2- |
| | yl)ethyl)piperidin-4-yl)benzoyl)- |
| | 4,5,6,7-tetrahydropyrazolo[1,5- |
| | a]pyrazin-2-yl)methyl)piperidin- |
| | 4-yl)phenyl)amino)piperidine- |
| | 2,6-dione |
| 2c43 | (R)-3-((3-fluoro-4-(1-((5-(4-(1- |
| | ((S)-1-(1-methyl-4-(6- |
| | (methylamino)pyridin-3-yl)-1H- |
| | pyrrolo[2,3-b]pyridin-2- |
| | yl)ethyl)piperidin-4-yl)benzoyl)- |
| | 4,5,6,7-tetrahydropyrazolo[1,5- |
| | a]pyrazin-2-yl)methyl)piperidin- |
| | 4-yl)phenyl)amino)piperidine- |
| | 2,6-dione |
| 2c44 | 3-((4-(1-((5-(4-(1-((S)-1-(4-(6- |
| | aminopyridin-3-yl)-1-methyl- |
| | 1H-pyrrolo[2,3-b]pyridin-2- |
| | yl)ethyl)piperidin-4-yl)benzoyl)- |
| | 4,5,6,7-tetrahydropyrazolo[1,5- |
| | a]pyrazin-2-yl)methyl)piperidin- |
| | 4-yl)-2- |
| | methoxyphenyl)amino)piperidine- |
| | 2,6-dione |
| 2c45 | 3-((5-fluoro-6-((S)-4-((1-(3- |
| | fluoro-5-methyl-4-(1-((S)-1-(1- |
| | methyl-4-(6-(methyl- |
| | amino)pyridin-3-yl)-1H- |
| | pyrrolo[2,3-b]pyridin-2- |
| | yl)ethyl)-1,2,3,6- |
| | tetrahydropyridin-4-yl)benzoyl)- |
| | piperidin-4-yl)methyl)-3- |
| | methylpiperazin-1-yl)pyridin-3- |
| | yl)amino)piperidine-2,6-dione |
| 2c46 | 3-((4-(4-((1-(3-fluoro-5-methyl- |
| | 4-(1-((S)-1-(1-methyl-4-(6- |
| | (methylamino)pyridin-3-yl)-1H- |
| | pyrrolo[2,3-b]pyridin-2- |
| | yl)ethyl)-1,2,3,6- |
| | tetrahydropyridin-4- |
| | yl)benzoyl)piperidin-4- |
| | yl)methyl)piperazin-1-yl)-2- |
| | methoxyphenyl)amino)piperidine- |
| | 2,6-dione |
| 2c47 | 3-((3-fluoro-4-((S)-4-((1-(3- |
| | fluoro-5-methyl-4-(1-((S)-1-(1- |
| | methyl-4-(6- |
| | (methylamino)pyridin-3-yl)-1H- |
| | pyrrolo[2,3-b]pyridin-2- |
| | yl)ethyl)-1,2,3,6- |
| | tetrahydropyridin-4- |
| | yl)benzoyl)piperidin-4- |
| | yl)methyl)-3-methylpiperazin-1- |
| | yl)phenyl)amino)piperidine-2,6- |
| | dione |
| 2c48 | 3-((4-(1-((5-(1′-((4-(6- |
| | aminopyridazin-3-yl)-1-methyl- |
| | 1H-pyrrolo[2,3-b]pyridin-2- |
| | yl)methyl)-3-methyl-1′,2′,3′,6′- |
| | tetrahydro-[2,4′-bipyridine]-5- |
| | carbonyl)-4,5,6,7- |
| | tetrahydropyrazolo[1,5- |
| | a]pyrazin-2-yl)methyl)piperidin- |
| | 4-yl)-3- |
| | fluorophenyl)amino)piperidine- |
| | 2,6-dione |
| 2c49 | 3-((4-(1-((5-(3-fluoro-5-methyl- |
| | 4-(1-((S)-1-(1-methyl-4-(6- |
| | (methylamino)pyridin-3-yl)-1H- |
| | pyrrolo[2,3-b]pyridin-2- |
| | yl)ethyl)-1,2,3,6- |
| | tetrahydropyridin-4-yl)benzoyl)- |
| | 4,5,6,7-tetrahydropyrazolo[1,5- |
| | a]pyrazin-2-yl)methyl)piperidin- |
| | 4-yl)-2- |
| | methoxyphenyl)amino)piperidine- |
| | 2,6-dione |
| 2c52 | (R)-3-((3-fluoro-4-(4-((1-(4-(1- |
| | ((S)-1-(4-(2-(methoxymethyl)- |
| | 1H-benzo[d]imidazol-5-yl)-1- |
| | methyl-1H-pyrrolo[2,3- |
| | b]pyridin-2-yl)ethyl)piperidin-4- |
| | yl)benzoyl)piperidin-4- |
| | yl)methyl)piperazin-1- |
| | yl)phenyl)amino)piperidine-2,6- |
| | dione |
| 2c53 | (S)-3-((3-fluoro-4-(4-((1-(4-(1- |
| | ((S)-1-(4-(2-(methoxymethyl)- |
| | 1H-benzo[d]imidazol-5-yl)-1- |
| | methyl-1H-pyrrolo[2,3- |
| | b]pyridin-2-yl)ethyl)piperidin-4- |
| | yl)benzoyl)piperidin-4- |
| | yl)methyl)piperazin-1- |
| | yl)phenyl)amino)piperidine-2,6- |
| | dione |
| 2c54 | 3-((4-((S)-4-((1-(4-(1-((S)-1-(4- |
| | (6-aminopyridin-3-yl)-1-methyl- |
| | 1H-pyrrolo[2,3-b]pyridin-2- |
| | yl)ethyl)piperidin-4- |
| | yl)benzoyl)piperidin-4- |
| | yl)methyl)-3-methylpiperazin-1- |
| | yl)-3- |
| | fluorophenyl)amino)piperidine- |
| | 2,6-dione |
| 2c55 | 3-((6-((S)-4-((1-(4-(1-((S)-1-(4- |
| | (6-aminopyridin-3-yl)-1-methyl- |
| | 1H-pyrrolo[2,3-b]pyridin-2- |
| | yl)ethyl)piperidin-4- |
| | yl)benzoyl)piperidin-4- |
| | yl)methyl)-3-methylpiperazin-1- |
| | yl)-5-fluoropyridin-3- |
| | yl)amino)piperidine-2,6-dione |
| 2c56 | 1-(1-(4-((4-((1-(4-(1-((4-(2- |
| | (methoxymethyl)-1H- |
| | benzo[d]imidazol-5-yl)-1- |
| | methyl-1H-pyrrolo[2,3- |
| | b]pyridin-2-yl)methyl)piperidin- |
| | 4-yl)benzoyl)piperidin-4- |
| | yl)methyl)piperazin-1- |
| | yl)methyl)-phenyl)-1H-1,2,3- |
| | triazol-4-yl)dihydropyrimidine- |
| | 2,4(1H,3H)-dione |
| 2c57 | 3-((4-(1-(2-(1-(3-fluoro-5- |
| | methyl-4-(1-((R)-1-(1-methyl-4- |
| | (6-(methylamino)pyridin-3-yl)- |
| | 1H-pyrrolo[2,3-b]pyridin-2- |
| | yl)ethyl)-1,2,3,6- |
| | tetrahydropyridin-4- |
| | yl)benzoyl)piperidin-4-yl)ethyl)- |
| | 1H-1,2,3-triazol-4- |
| | yl)phenyl)amino)piperidine-2,6- |
| | dione |
| 2d1 | 6-(4-((1-(4-(1-((4-(1H-indazol-5- |
| | yl)-1-methyl-1H-pyrrolo[2,3- |
| | b]pyridin-2-yl)methyl)- |
| | piperidin-4-yl)benzoyl)piperidin- |
| | 4-yl)methyl)piperazin-1-yl)-N- |
| | (2,6-dioxopiperidin-3- |
| | yl)pyridazine-3-carboxamide |
| 2e1 | 3-((4-(4-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperazine-1- |
| | carbonyl)-3- |
| | fluorophenyl)amino)piperidine- |
| | 2,6-dione |
| 2e2 | 3-((4-(4-((1-(4-(1-((4-(6- |
| | aminopyridin-3-yl)-1-methyl- |
| | 1H-pyrrolo[2,3-b]pyridin-2- |
| | yl)methyl)-piperidin-4- |
| | yl)benzoyl)piperidin-4- |
| | yl)methyl)piperazine-1- |
| | carbonyl)-3- |
| | fluorophenyl)amino)piperidine- |
| | 2,6-dione |
| 2f1 | 3-((3-fluoro-4-((R)-4-((1-(4-(1- |
| | ((S)-1-(4-(2-(methoxymethyl)- |
| | 1H-benzo[d]imidazol-5-yl)-1- |
| | methyl-1H-pyrrolo[2,3- |
| | b]pyridin-2-yl)ethyl)piperidin-4- |
| | yl)benzoyl)piperidin-4- |
| | yl)methyl)-3-methylpiperazin-1- |
| | yl)phenyl)amino)piperidine-2,6- |
| | dione |
| 2f2 | 3-((4-((R)-4-((1-(4-(1-((S)-1-(4- |
| | (6-aminopyridin-3-yl)-1-methyl- |
| | 1H-pyrrolo[2,3-b]pyridin-2- |
| | yl)ethyl)piperidin-4- |
| | yl)benzoyl)piperidin-4- |
| | yl)methyl)-3-methylpiperazin-1- |
| | yl)-3-fluorophenyl)amino)- |
| | piperidine-2,6-dione |
| 2f3 | 3-((4-((S)-4-((1-(4-(1-((4-(6- |
| | aminopyridin-3-yl)-1-methyl- |
| | 1H-pyrrolo[2,3-b]pyridin-2- |
| | yl)methyl)piperidin-4- |
| | yl)benzoyl)piperidin-4- |
| | yl)methyl)-3-methylpiperazin-1- |
| | yl)-3-fluorophenyl)amino)- |
| | piperidine-2,6-dione |
| 2f4 | 3-((3-fluoro-4-((S)-4-((1-(4-(1- |
| | ((4-(2-(methoxymethyl)-1H- |
| | benzo[d]imidazol-5-yl)-1- |
| | methyl-1H-pyrrolo[2,3- |
| | b]pyridin-2-yl)methyl)piperidin- |
| | 4-yl)benzoyl)piperidin-4- |
| | yl)methyl)-3-methylpiperazin-1- |
| | yl)phenyl)-amino)piperidine-2,6- |
| | dione |
| 2f5 | 3-((4-((S)-4-((1-(4-(1-((4-(1H- |
| | indazol-5-yl)-1-methyl-1H- |
| | pyrrolo[2,3-b]pyridin-2- |
| | yl)methyl)piperidin-4- |
| | yl)benzoyl)piperidin-4- |
| | yl)methyl)-3-methylpiperazin-1- |
| | yl)-3- |
| | fluorophenyl)amino)piperidine- |
| | 2,6-dione |
| 2f6 | 3-((4-((R)-4-((1-(4-(1-((4-(6- |
| | aminopyridin-3-yl)-1-methyl- |
| | 1H-pyrrolo[2,3-b]pyridin-2-yl)- |
| | methyl)-piperidin-4- |
| | yl)benzoyl)piperidin-4- |
| | yl)methyl)-3-methylpiperazin-1- |
| | yl)-3-fluorophenyl)amino)- |
| | piperidine-2,6-dione |
| 2f7 | 3-((3-fluoro-4-((R)-4-((1-(4-(1- |
| | ((4-(2-(methoxymethyl)-1H- |
| | benzo[d]imidazol-5-yl)-1- |
| | methyl-1H-pyrrolo[2,3- |
| | b]pyridin-2-yl)methyl)piperidin- |
| | 4-yl)benzoyl)piperidin-4- |
| | yl)methyl)-3-methylpiperazin-1- |
| | yl)phenyl)-amino)piperidine-2,6- |
| | dione |
| 2f8 | 3-((4-((R)-4-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)-3-methylpiperazin-1- |
| | yl)-3- |
| | fluorophenyl)amino)piperidine- |
| | 2,6-dione |
| 2f9 | 3-((4-(4-((1-(4-(1-((S)-1-(4-(6- |
| | aminopyridin-3-yl)-1-methyl- |
| | 1H-pyrrolo[2,3-b]pyridin-2-yl)- |
| | ethyl)piperidin-4- |
| | yl)benzoyl)piperidin-4- |
| | yl)methyl)-3-(trifluoromethyl)- |
| | piperazin-1-yl)-3-fluorophenyl)- |
| | amino)piperidine-2,6-dione |
| 2f10 | 3-((4-((S)-4-((1-(4-(1-((S)-1-(4- |
| | (6-aminopyridin-3-yl)-1-methyl- |
| | 1H-pyrrolo[2,3-b]pyridin-2- |
| | yl)ethyl)piperidin-4- |
| | yl)benzoyl)piperidin-4- |
| | yl)methyl)-3- |
| | (trifluoromethyl)piperazin-1-yl)- |
| | 3-fluorophenyl)- |
| | amino)piperidine-2,6-dione |
| 2f11 | 3-((4-((R)-4-((1-(4-(1-((S)-1-(4- |
| | (6-aminopyridin-3-yl)-1-methyl- |
| | 1H-pyrrolo[2,3-b]pyridin-2- |
| | yl)ethyl)piperidin-4- |
| | yl)benzoyl)piperidin-4- |
| | yl)methyl)-3- |
| | (trifluoromethyl)piperazin-1-yl)- |
| | 3-fluoro- |
| | phenyl)amino)piperidine-2,6- |
| | dione |
| 2f12 | (S)-3-((3-fluoro-4-((R)-4-((1-(4- |
| | (1-((S)-1-(4-(2- |
| | (methoxymethyl)-1H-benzo[d]- |
| | imidazol-5-yl)-1-methyl-1H- |
| | pyrrolo[2,3-b]pyridin-2- |
| | yl)ethyl)piperidin-4- |
| | yl)benzoyl)piperidin-4-yl)- |
| | methyl)-3-methylpiperazin-1- |
| | yl)phenyl)amino)piperidine-2,6- |
| | dione |
| 2f13 | (R)-3-((3-fluoro-4-((R)-4-((1-(4- |
| | (1-((S)-1-(4-(2- |
| | (methoxymethyl)-1H- |
| | benzo[d]imidazol-5-yl)-1- |
| | methyl-1H-pyrrolo[2,3- |
| | b]pyridin-2-yl)-ethyl)piperidin- |
| | 4-yl)benzoyl)piperidin-4- |
| | yl)methyl)-3-methylpiperazin-1- |
| | yl)phenyl)amino)piperidine-2,6- |
| | dione |
| 2g1 | 3-((1-(1-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperidin-4-yl)-1H- |
| | indazol-4-yl)amino)piperidine- |
| | 2,6-dione |
| 2g63 | 3-((4-(4-((4-(4-(1-((S)-1-(4-(6- |
| | aminopyridin-3-yl)-1-methyl- |
| | 1H-pyrrolo[2,3-b]pyridin-2- |
| | yl)ethyl)piperidin-4- |
| | yl)benzoyl)piperazin-1- |
| | yl)methyl)piperidin-1-yl)-3- |
| | fluorophenyl)amino)piperidine- |
| | 2,6-dione |
| 2h1 | 3-((4-(1′-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)-3′,3′-difluoro-[1,4′- |
| | bipiperidin]-4-yl)-3- |
| | fluorophenyl)-amino)piperidine- |
| | 2,6-dione |
| 2h2 | 3-((4-(1′-((1-(4-(1-((4-(6- |
| | aminopyridin-3-yl)-1-methyl- |
| | 1H-pyrrolo[2,3-b]pyridin-2-yl)- |
| | methyl)piperidin-4- |
| | yl)benzoyl)piperidin-4- |
| | yl)methyl)-3′,3′-difluoro-[1,4′- |
| | bipiperidin]-4-yl)-3-fluoro- |
| | phenyl)amino)piperidine-2,6- |
| | dione |
| 2h3 | 3-((4-(3′,3′-difluoro-1′-((1-(4-(1- |
| | ((4-(2-(methoxymethyl)-1H- |
| | benzo[d]imidazol-5-yl)-1- |
| | methyl-1H-pyrrolo[2,3- |
| | b]pyridin-2-yl)methyl)piperidin- |
| | 4-yl)benzoyl)piperidin-4- |
| | yl)methyl)-[1,4′-bipiperidin]-4- |
| | yl)-3- |
| | fluorophenyl)amino)piperidine- |
| | 2,6-dione |
| 2i1 | 3-((4-(4-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperazin-1-yl)-5- |
| | fluoro-2-methoxyphenyl)amino)- |
| | piperidine-2,6-dione |
| 2i2 | 5-((2,6-dioxopiperidin-3- |
| | yl)amino)-2-(4-((1-(4-(1-((S)-1- |
| | (4-(2-(methoxymethyl)-1H- |
| | benzo[d]-imidazol-5-yl)-1- |
| | methyl-1H-pyrrolo[2,3- |
| | b]pyridin-2-yl)ethyl)piperidin-4- |
| | yl)benzoyl)piperidin-4- |
| | yl)methyl)-piperazin-1- |
| | yl)benzonitrile |
| 2i3 | 2-(4-((1-(4-(1-((S)-1-(4-(6- |
| | aminopyridin-3-yl)-1-methyl- |
| | 1H-pyrrolo[2,3-b]pyridin-2-yl)- |
| | ethyl)-piperidin-4- |
| | yl)benzoyl)piperidin-4- |
| | yl)methyl)piperazin-1-yl)-5- |
| | ((2,6-dioxopiperidin-3- |
| | yl)amino)-benzonitrile |
| 2i4 | 3-((3-chloro-4-(4-((1-(4-(1-((S)- |
| | 1-(4-(2-(methoxymethyl)-1H- |
| | benzo[d]imidazol-5-yl)-1- |
| | methyl-1H-pyrrolo[2,3- |
| | b]pyridin-2-yl)ethyl)piperidin-4- |
| | yl)benzoyl)piperidin-4- |
| | yl)methyl)piperazin-1- |
| | yl)phenyl)amino)piperidine-2,6- |
| | dione |
| 2i5 | 3-((4-(4-((1-(4-(1-((S)-1-(4-(2- |
| | (methoxymethyl)-1H- |
| | benzo[d]imidazol-5-yl)-1- |
| | methyl-1H-pyrrolo[2,3- |
| | b]pyridin-2-yl)ethyl)piperidin-4- |
| | yl)benzoyl)piperidin-4- |
| | yl)methyl)piperazin-1-yl)-3- |
| | (trifluoromethyl)phenyl)amino)p |
| | iperidine-2,6-dione |
| 2i6 | 3-((4-(4-((1-(4-(1-((S)-1-(4-(6- |
| | aminopyridin-3-yl)-1-methyl- |
| | 1H-pyrrolo[2,3-b]pyridin-2-yl)- |
| | ethyl)piperidin-4- |
| | yl)benzoyl)piperidin-4- |
| | yl)methyl)piperazin-1-yl)-3- |
| | chlorophenyl)amino)piperidine- |
| | 2,6-dione |
| 2i7 | 3-((4-(4-((1-(4-(1-((S)-1-(4-(6- |
| | aminopyridin-3-yl)-1-methyl- |
| | 1H-pyrrolo[2,3-b]pyridin-2-yl)- |
| | ethyl)-piperidin-4- |
| | yl)benzoyl)piperidin-4- |
| | yl)methyl)piperazin-1-yl)-3- |
| | (trifluoromethyl)phenyl)amino)- |
| | piperidine-2,6-dione |
| 2j1 | 4-(1-((4-(1H-indazol-5-yl)-1- |
| | methyl-1H-pyrrolo[2,3- |
| | b]pyridin-2-yl)methyl)piperidin- |
| | 4-yl)-N-(4-(1-(4-((2,6- |
| | dioxopiperidin-3-yl)amino)-2- |
| | fluorophenyl)piperidin-4- |
| | yl)phenyl)-N-methylbenzamide |
| 2j2 | 4-(1-((4-(1H-indazol-5-yl)-1- |
| | methyl-1H-pyrrolo[2,3- |
| | b]pyridin-2-yl)methyl)piperidin- |
| | 4-yl)-N-(4-(1-(4-((2,6- |
| | dioxopiperidin-3- |
| | yl)amino)phenyl)piperidin-4- |
| | yl)phenyl)-N-methylbenzamide |
| 2k1 | 3-((2-(4-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperazin-1- |
| | yl)pyrimidin-5- |
| | yl)amino)piperidine-2,6-dione |
| 2k2 | 3-((2-(4-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperazin-1- |
| | yl)pyrimidin-4- |
| | yl)amino)piperidine-2,6-dione |
| 2k3 | 3-((4-(4-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperazin-1- |
| | yl)pyrimidin-2- |
| | yl)amino)piperidine-2,6-dione |
| 2l1 | 3-((3-(4-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperazin-1- |
| | yl)phenyl)amino)piperidine-2,6- |
| | dione |
| 2m1 | 3-((4-(1-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperidin-4-yl)-3- |
| | fluorophenyl)amino)piperidine- |
| | 2,6-dione |
| 2m2 | 3-((4-(1-((1-(4-(1-((S)-1-(4-(6- |
| | aminopyridin-3-yl)-1-methyl- |
| | 1H-pyrrolo[2,3-b]pyridin-2-yl)- |
| | ethyl)piperidin-4- |
| | yl)benzoyl)piperidin-4- |
| | yl)methyl)-3,3-difluoropiperidin- |
| | 4-yl)-3-fluorophenyl)amino)- |
| | piperidine-2,6-dione |
| 2m3 | 3-((4-(3,3-difluoro-1-((1-(4-(1- |
| | ((S)-1-(4-(2-(methoxymethyl)- |
| | 1H-benzo[d]imidazol-5-yl)-1- |
| | methyl-1H-pyrrolo[2,3- |
| | b]pyridin-2-yl)ethyl)piperidin-4- |
| | yl)benzoyl)piperidin-4- |
| | yl)methyl)piperidin-4-yl)-3- |
| | fluorophenyl)amino)piperidine- |
| | 2,6-dione |
| 2n1 | 3-((6-(4-((1-(4-(1-((4-(1H- |
| | indazol-5-yl)-1-methyl-1H- |
| | pyrrolo[2,3-b]pyridin-2- |
| | yl)methyl)piperidin-4- |
| | yl)benzoyl)piperidin-4- |
| | yl)methyl)piperazin-1- |
| | yl)pyridin-3- |
| | yl)amino)piperidine-2,6-dione |
| 2n10 | 3-((6-(4-((1-(4-(1-((S)-1-(4-(6- |
| | aminopyridin-3-yl)-1-methyl- |
| | 1H-pyrrolo[2,3-b]pyridin-2- |
| | yl)ethyl)-piperidin-4- |
| | yl)benzoyl)piperidin-4- |
| | yl)methyl)piperazin-1-yl)-5- |
| | fluoropyridin-3-yl)amino)- |
| | piperidine-2,6-dione |
| 2n11 | 3-((2-fluoro-6-(4-((1-(4-(1-((S)- |
| | 1-(4-(2-(methoxymethyl)-1H- |
| | benzo[d]-imidazol-5-yl)-1- |
| | methyl-1H-pyrrolo[2,3- |
| | b]pyridin-2-yl)ethyl)piperidin-4- |
| | yl)benzoyl)-piperidin-4- |
| | yl)methyl)piperazin-1- |
| | yl)pyridin-3- |
| | yl)amino)piperidine-2,6-dione |
| 2n12 | 3-((5-fluoro-6-(4-((1-(4-(1-((S)- |
| | 1-(4-(2-(methoxymethyl)-1H- |
| | benzo[d]imidazol-5-yl)-1- |
| | methyl-1H-pyrrolo[2,3- |
| | b]pyridin-2-yl)ethyl)piperidin-4- |
| | yl)benzoyl)piperidin-4- |
| | yl)methyl)piperazin-1- |
| | yl)pyridin-3- |
| | yl)amino)piperidine-2,6-dione |
| 2n13 | 3-((5-fluoro-6-(4-((1-(4-(1-((S)- |
| | 1-(1-methyl-4-(6- |
| | (methylamino)pyridin-3-yl)-1H- |
| | pyrrolo[2,3-b]pyridin-2- |
| | yl)ethyl)piperidin-4- |
| | yl)benzoyl)piperidin-4- |
| | yl)methyl)-piperazin-1- |
| | yl)pyridin-3- |
| | yl)amino)piperidine-2,6-dione |
| 2n14 | 3-((2-fluoro-6-(4-((1-(4-(1-((S)- |
| | 1-(1-methyl-4-(6- |
| | (methylamino)pyridin-3-yl)-1H- |
| | pyrrolo[2,3-b]pyridin-2- |
| | yl)ethyl)piperidin-4- |
| | yl)benzoyl)piperidin-4- |
| | yl)methyl)piperazin-1- |
| | yl)pyridin-3- |
| | yl)amino)piperidine-2,6-dione |
| 2n2 | 3-((6-(4-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperazin-1-yl)-2- |
| | fluoropyridin-3- |
| | yl)amino)piperidine-2,6-dione |
| 2n3 | 3-((2-fluoro-6-(4-((1-(4-(1-((4- |
| | (2-(methoxymethyl)-1H- |
| | benzo[d]imidazol-5-yl)-1- |
| | methyl-1H-pyrrolo[2,3- |
| | b]pyridin-2-yl)methyl)piperidin- |
| | 4-yl)benzoyl)piperidin-4- |
| | yl)methyl)piperazin-1- |
| | yl)pyridin-3- |
| | yl)amino)piperidine-2,6-dione |
| 2n4 | 3-((6-(4-((1-(4-(1-((4-(6- |
| | aminopyridin-3-yl)-1-methyl- |
| | 1H-pyrrolo[2,3-b]pyridin-2- |
| | yl)methyl)-piperidin-4- |
| | yl)benzoyl)piperidin-4- |
| | yl)methyl)piperazin-1-yl)-2- |
| | fluoropyridin-3- |
| | yl)amino)piperidine-2,6-dione |
| 2n5 | 3-((6-(4-((1-(4-(1-((4-(6- |
| | aminopyridin-3-yl)-1-methyl- |
| | 1H-pyrrolo[2,3-b]pyridin-2- |
| | yl)methyl)-piperidin-4- |
| | yl)benzoyl)piperidin-4- |
| | yl)methyl)piperazin-1-yl)-5- |
| | fluoropyridin-3- |
| | yl)amino)piperidine-2,6-dione |
| 2n6 | 3-((5-fluoro-6-(4-((1-(4-(1-((4- |
| | (2-(methoxymethyl)-1H- |
| | benzo[d]imidazol-5-yl)-1- |
| | methyl-1H-pyrrolo[2,3- |
| | b]pyridin-2-yl)methyl)piperidin- |
| | 4-yl)benzoyl)piperidin-4- |
| | yl)methyl)piperazin-1- |
| | yl)pyridin-3- |
| | yl)amino)piperidine-2,6-dione |
| 2n7 | 3-((6-(4-((1-(4-(1-((S)-1-(4-(1H- |
| | indazol-5-yl)-1-methyl-1H- |
| | pyrrolo[2,3-b]pyridin-2- |
| | yl)ethyl)-piperidin-4- |
| | yl)benzoyl)piperidin-4- |
| | yl)methyl)piperazin-1-yl)-2- |
| | fluoropyridin-3- |
| | yl)amino)piperidine-2,6-dione |
| 2n8 | 3-((6-(4-((1-(4-(1-((S)-1-(4-(1H- |
| | indazol-5-yl)-1-methyl-1H- |
| | pyrrolo[2,3-b]pyridin-2- |
| | yl)ethyl)-piperidin-4- |
| | yl)benzoyl)piperidin-4- |
| | yl)methyl)piperazin-1-yl)-5- |
| | fluoropyridin-3- |
| | yl)amino)piperidine-2,6-dione |
| 2n9 | 3-((6-(4-((1-(4-(1-((S)-1-(4-(6- |
| | aminopyridin-3-yl)-1-methyl- |
| | 1H-pyrrolo[2,3-b]pyridin-2-yl)- |
| | ethyl)-piperidin-4- |
| | yl)benzoyl)piperidin-4- |
| | yl)methyl)piperazin-1-yl)-2- |
| | fluoropyridin-3- |
| | yl)amino)piperidine-2,6-dione |
| 2o1 | 4-(1-((4-(6-aminopyridin-3-yl)- |
| | 1-methyl-1H-pyrrolo[2,3- |
| | b]pyridin-2-yl)- |
| | methyl)piperidin-4-yl)-N-(4-((1- |
| | (4-((2,6-dioxopiperidin-3- |
| | yl)amino)phenyl)piperidin-4- |
| | yl)methyl)phenyl)-N- |
| | methylbenzamide |
| 2o2 | 4-(1-((4-(1H-indazol-5-yl)-1- |
| | methyl-1H-pyrrolo[2,3- |
| | b]pyridin-2-yl)methyl)- |
| | piperidin-4-yl)-N-(4-((1-(4-((2,6- |
| | dioxopiperidin-3- |
| | yl)amino)phenyl)piperidin-4- |
| | yl)methyl)-phenyl)-N- |
| | methylbenzamide |
| 2p1 | 3-((4-(4-(1-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)-3,3-difluoropiperidin- |
| | 4-yl)piperazin-1-yl)-3- |
| | fluorophenyl)-amino)piperidine- |
| | 2,6-dione |
| 2p2 | 3-((4-(4-(1-((1-(4-(1-((4-(6- |
| | aminopyridin-3-yl)-1-methyl- |
| | 1H-pyrrolo[2,3-b]pyridin-2-yl)- |
| | methyl)piperidin-4- |
| | yl)benzoyl)piperidin-4- |
| | yl)methyl)-3,3-difluoropiperidin- |
| | 4-yl)piperazin-1-yl)-3- |
| | fluorophenyl)amino)piperidine- |
| | 2,6-dione |
| 2p3 | 3-((4-(4-(3,3-difluoro-1-((1-(4- |
| | (1-((4-(2-(methoxymethyl)-1H- |
| | benzo[d]imidazol-5-yl)-1- |
| | methyl-1H-pyrrolo[2,3- |
| | b]pyridin-2-yl)methyl)piperidin- |
| | 4-yl)benzoyl)piperidin-4- |
| | yl)methyl)piperidin-4- |
| | yl)piperazin-1-yl)-3- |
| | fluorophenyl)amino)piperidine- |
| | 2,6-dione |
| 2q1 | 3-((4-(1-((7-(4-(1-((4-(6- |
| | aminopyridin-3-yl)-1-methyl- |
| | 1H-pyrrolo[2,3-b]pyridin-2- |
| | yl)methyl)piperidin-4- |
| | yl)benzoyl)-5,6,7,8-tetrahydro- |
| | [1,2,4]triazolo[4,3-a]pyrazin-3- |
| | yl)methyl)piperidin-4-yl)-3- |
| | fluorophenyl)amino)piperidine- |
| | 2,6-dione |
| 2q2 | 3-((4-(1-((7-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)-5,6,7,8-tetrahydro- |
| | [1,2,4]triazolo[4,3-a]pyrazin-3- |
| | yl)methyl)piperidin-4-yl)-3- |
| | fluorophenyl)amino)piperidine- |
| | 2,6-dione |
| 2q3 | 3-((4-(1-((5-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)-4,5,6,7- |
| | tetrahydropyrazolo[1,5- |
| | a]pyrazin-2-yl)methyl)piperidin- |
| | 4-yl)-3- |
| | fluorophenyl)amino)piperidine- |
| | 2,6-dione |
| 2q4 | 3-((4-(1-((5-(4-(1-((4-(6- |
| | aminopyridin-3-yl)-1-methyl- |
| | 1H-pyrrolo[2,3-b]pyridin-2- |
| | yl)methyl)-piperidin-4- |
| | yl)benzoyl)-4,5,6,7- |
| | tetrahydropyrazolo[1,5- |
| | a]pyrazin-2-yl)methyl)piperidin- |
| | 4-yl)-3-fluoro- |
| | phenyl)amino)piperidine-2,6- |
| | dione |
| 2q5 | 3-((4-(1-((5-(4-(1-((S)-1-(4-(6- |
| | aminopyridin-3-yl)-1-methyl- |
| | 1H-pyrrolo[2,3-b]pyridin-2- |
| | yl)ethyl)-piperidin-4- |
| | yl)benzoyl)-4,5,6,7- |
| | tetrahydropyrazolo[1,5- |
| | a]pyrazin-2-yl)methyl)piperidin- |
| | 4-yl)-3-fluorophenyl)- |
| | amino)piperidine-2,6-dione |
| 2q6 | 3-((6-(1-((5-(4-(1-((S)-1-(4-(6- |
| | aminopyridin-3-yl)-1-methyl- |
| | 1H-pyrrolo[2,3-b]pyridin-2- |
| | yl)ethyl)-piperidin-4- |
| | yl)benzoyl)-4,5,6,7- |
| | tetrahydropyrazolo[1,5- |
| | a]pyrazin-2-yl)methyl)piperidin- |
| | 4-yl)-5-fluoropyridin-3- |
| | yl)amino)piperidine-2,6-dione |
| 2q7 | 3-((3-fluoro-4-(1-((5-(4-(1-((4- |
| | (2-(methoxymethyl)-1H- |
| | benzo[d]imidazol-5-yl)-1- |
| | methyl-1H-pyrrolo[2,3- |
| | b]pyridin-2-yl)methyl)piperidin- |
| | 4-yl)benzoyl)-4,5,6,7- |
| | tetrahydropyrazolo[1,5- |
| | a]pyrazin-2-yl)- |
| | methyl)piperidin-4- |
| | yl)phenyl)amino)piperidine-2,6- |
| | dione |
| 2q8 | 3-((5-fluoro-6-(1-((5-(4-(1-((4- |
| | (2-(methoxymethyl)-1H- |
| | benzo[d]imidazol-5-yl)-1- |
| | methyl-1H-pyrrolo[2,3- |
| | b]pyridin-2-yl)methyl)piperidin- |
| | 4-yl)benzoyl)-4,5,6,7- |
| | tetrahydropyrazolo[1,5- |
| | a]pyrazin-2-yl)methyl)piperidin- |
| | 4-yl)pyridin-3- |
| | yl)amino)piperidine-2,6-dione |
| 2q9 | 3-((4-(1-((5-(4-(1-((S)-1-(4-(6- |
| | aminopyridin-3-yl)-1-methyl- |
| | 1H-pyrrolo[2,3-b]pyridin-2-yl)- |
| | ethyl)piperidin-4-yl)benzoyl)- |
| | 4,5,6,7-tetrahydropyrazolo[1,5- |
| | a]pyrazin-2-yl)methyl)-3,3- |
| | difluoro-piperidin-4-yl)-3- |
| | fluorophenyl)amino)piperidine- |
| | 2,6-dione |
| 2q10 | (R)-3-((4-(1-((5-(4-(1-((S)-1-(4- |
| | (6-aminopyridin-3-yl)-1-methyl- |
| | 1H-pyrrolo[2,3-b]pyridin-2- |
| | yl)ethyl)piperidin-4-yl)benzoyl)- |
| | 4,5,6,7-tetrahydropyrazolo[1,5- |
| | a]pyrazin-2-yl)methyl)piperidin- |
| | 4-yl)-3- |
| | fluorophenyl)amino)piperidine- |
| | 2,6-dione |
| 2q11 | (S)-3-((4-(1-((5-(4-(1-((S)-1-(4- |
| | (6-aminopyridin-3-yl)-1-methyl- |
| | 1H-pyrrolo[2,3-b]pyridin-2- |
| | yl)ethyl)piperidin-4-yl)benzoyl)- |
| | 4,5,6,7-tetrahydropyrazolo[1,5- |
| | a]pyrazin-2-yl)methyl)piperidin- |
| | 4-yl)-3- |
| | fluorophenyl)amino)piperidine- |
| | 2,6-dione |
| 2q12 | 3-((4-(3,3-difluoro-1-((5-(4-(1- |
| | ((4-(2-(methoxymethyl)-1H- |
| | benzo[d]imidazol-5-yl)-1- |
| | methyl-1H-pyrrolo[2,3- |
| | b]pyridin-2-yl)methyl)piperidin- |
| | 4-yl)benzoyl)-4,5,6,7- |
| | tetrahydropyrazolo[1,5- |
| | a]pyrazin-2-yl)methyl)piperidin- |
| | 4-yl)-3- |
| | fluorophenyl)amino)piperidine- |
| | 2,6-dione |
| 2q14 | 3-((6-(4-((5-(4-(1-((S)-1-(4-(6- |
| | aminopyridin-3-yl)-1-methyl- |
| | 1H-pyrrolo[2,3-b]pyridin-2- |
| | yl)ethyl)-piperidin-4- |
| | yl)benzoyl)-4,5,6,7- |
| | tetrahydropyrazolo[1,5- |
| | a]pyrazin-2-yl)methyl)piperazin- |
| | 1-yl)-5-fluoropyridin-3- |
| | yl)amino)piperidine-2,6-dione |
| 2q15 | 3-((4-(4-((5-(4-(1-((S)-1-(4-(6- |
| | aminopyridin-3-yl)-1-methyl- |
| | 1H-pyrrolo[2,3-b]pyridin-2-yl)- |
| | ethyl)piperidin-4-yl)benzoyl)- |
| | 4,5,6,7-tetrahydropyrazolo[1,5- |
| | a]pyrazin-2-yl)methyl)piperazin- |
| | 1-yl)-3-fluoro- |
| | phenyl)amino)piperidine-2,6- |
| | dione |
| 2r1 | 3-((4-(4-((1-(3-(4-(1-((4-(6- |
| | aminopyridin-3-yl)-1-methyl- |
| | 1H-pyrrolo[2,3-b]pyridin-2- |
| | yl)methyl)-piperidin-4- |
| | yl)phenyl)-[1,2,4]triazolo[4,3- |
| | b]pyridazin-6-yl)piperidin-4- |
| | yl)methyl)piperazin-1-yl)-3- |
| | fluorophenyl)-amino)piperidine- |
| | 2,6-dione |
| 2s1 | 3-((4-((4-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperazin-1- |
| | yl)methyl)phenyl)amino)piperidine- |
| | 2,6-dione |
| 2s2 | 3-((4-((4-((1-(4-(1-((4-(6- |
| | aminopyridin-3-yl)-1-methyl- |
| | 1H-pyrrolo[2,3-b]pyridin-2- |
| | yl)methyl)piperidin-4- |
| | yl)benzoyl)piperidin-4- |
| | yl)methyl)piperazin-1- |
| | yl)methyl)-3- |
| | fluorophenyl)amino)piperidine- |
| | 2,6-dione |
| 2t1 | 3-(4-(1-(2-(4-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperazin-1-yl)-2- |
| | oxoethyl)piperidin-4-yl)-3- |
| | fluorophenoxy)piperidine-2,6- |
| | dione |
| 2t2 | 3-((2-(4-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperazine-1- |
| | carbonyl)phenyl)amino)piperidine- |
| | 2,6-dione |
| 2t3 | 3-((5-(4-((1-(4-(1-((4-(1H- |
| | indazol-5-yl)-1-methyl-1H- |
| | pyrrolo[2,3-b]pyridin-2- |
| | yl)methyl)piperidin-4- |
| | yl)benzoyl)piperidin-4- |
| | yl)methyl)piperazin-1- |
| | yl)pyridin-2- |
| | yl)amino)piperidine-2,6-dione |
| 2t4 | 3-((1-(1-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)methyl)piperidin-4-yl)-1H- |
| | indazol-5-yl)amino)piperidine- |
| | 2,6-dione |
| 2t5 | 3-((3-(3-((1-(4-(1-((4-(1H- |
| | indazol-5-yl)-1-methyl-1H- |
| | pyrrolo[2,3-b]pyridin-2- |
| | yl)methyl)-piperidin-4- |
| | yl)benzoyl)piperidin-4- |
| | yl)oxy)prop-1-yn-1- |
| | yl)phenyl)amino)-piperidine-2,6- |
| | dione |
| 3t5 | ((S)-1-(4-(2-(methoxymethyl)- |
| | 1H-benzo[d]imidazol-5-yl)-1- |
| | methyl-1H-3-((5-fluoro-6-((S)-4- |
| | ((1-(4-(1-((S)-1-(4-(2- |
| | (methoxymethyl)-1H- |
| | benzo[d]imidazol-5-yl)-1- |
| | methyl-1H-pyrrolo[2,3- |
| | b]pyridin-2-yl)ethyl)piperidin-4- |
| | yl)benzoyl)piperidin-4- |
| | yl)methyl)-3-methylpiperazin-1- |
| | yl)pyridin-3- |
| | yl)amino)piperidine-2,6-dione |

In one or more embodiments of the present disclosure, the compounds of formula (I) have an (EC₅₀) value in a Eotaxin-3 release assay of less than 100 micromolar, or of less than 10 micromolar, or less than 100 nanomolar, or less than 10 nanomolar.

In one or more embodiments of the present disclosure, the compounds of formula (I) have an (K_{D}) value in a Surface plasmon resonance (SPR) assay of less than 10 micromolar, or of less than 1 micromolar, or less than 100 nanomolar.

The compounds of formula (I) may be obtained in crystalline form either directly by concentration from an organic solvent or by crystallisation or recrystallisation from an organic solvent or mixture of said solvent and a co-solvent that may be organic or inorganic, such as water. The crystals may be isolated in essentially solvent-free form or as a solvate, such as a hydrate. The disclosure also provides crystalline forms of compounds of the disclosure, such as polymorphs and pseudopolymorphs, and also mixtures thereof.

Compounds of formula (I) may comprise asymmetrically substituted (chiral) carbon atoms which give rise to the existence of isomeric forms, e.g. enantiomers, diastereomers, and other stereoisomeric forms that can be defined, in terms of absolute stereochemistry, as (R)- or (S)-. The present disclosure provides all such isomers, either in optically pure form or as mixtures thereof (e.g. racemic mixtures or partially purified optical mixtures).

Pure stereoisomeric forms of the compounds and the intermediates of this disclosure may be obtained by the application of procedures known in the art. The various isomeric forms may be separated by physical separation methods such as selective crystallization and chromatographic techniques, e.g. high pressure liquid chromatography using chiral stationary phases. Enantiomers may be separated from each other by selective crystallization of their diastereomeric salts which may be formed with optically active amines, or with optically active acids. Optically purified compounds may subsequently be liberated from said purified diastereomeric salts. Enantiomers may also be resolved by the formation of diastereomeric derivatives. Alternatively, enantiomers may be separated by chromatographic techniques using chiral stationary phases. Pure stereoisomeric forms may also be derived from the corresponding pure stereoisomeric forms of the appropriate starting materials, provided that the reaction occur stereoselectively or stereospecifically. Preferably, if a specific stereoisomer is desired, said compound will be synthesized by stereoselective or stereospecific methods of preparation. These methods will advantageously employ chiral pure starting materials.

Furthermore, when a double bond or a fully or partially saturated ring system is present in the molecule geometric isomers may be formed. Any geometric isomer, as separated, pure or partially purified geometric isomers or mixtures thereof are included within the scope of the disclosure. When the compounds described herein contain olefinic double bonds or other centers of geometric asymmetry, and un1 ess specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

“Racemates” refers to a mixture of enantiomers. The mixture can comprise equal or unequal amounts of each enantiomer.

“STAT6 modulator” refers to compounds of the present disclosure that inhibit or reduce some or all of the activity of the Signal transducer and activator of transcription 6 (STAT6), including STAT6 degraders.

“STAT6 degrader” refers to compounds of the present disclosure that bind to and/or inhibit both STAT6 protein and an E3 ligase with measurable affinity, resulting in the ubiquitination and subsequent degradation of the STAT6 protein. In certain embodiments, a STAT6 degrader has an DC₅₀ of less than about 50 pM, less than about 1 pM, less than about 500 nM, less than about 100 nM, less than about 10 nM, or less than about 1 nM. As used herein, the term “monovalent” refers to a degrader compound without an appended E3 ligase binding moiety.

STAT6-mediated disorders, diseases, and/or conditions refers to any disease or other deleterious condition in which STAT6 or a mutant thereof, are known to play a role.

A “subject” or “patient” is meant to describe a human or vertebrate animal, including a dog, cat, horse, cow, mouse, or the like.

“Treatment” or “treating” is an approach for obtaining beneficial or desired results including clinical results, such as inhibiting the disease or condition (e.g., decreasing one or more symptoms resulting from the disease or condition, and/or diminishing the extent of the disease or condition), slowing or arresting the development of one or more clinical symptoms associated with the disease or condition, and/or relieving the disease, enhancing the effect of another medication, delaying the progression of the disease, increasing quality of life, and/or prolonging survival.

In the compounds of formula (I), the atoms may exhibit their natural isotopic abundances, or one or more of the atoms may be artificially enriched in a particular isotope having the same atomic number, but an atomic mass or mass number different from the atomic mass or mass number found in nature. The present disclosure includes all suitable isotopic variations of the compounds of formula (I). For example, different isotopic forms of hydrogen include 1H. 2H and 3H, different isotopic forms of carbon include 12C, 13C and 14C and different isotopic forms of nitrogen include 14N and 15N. Enriching for deuterium (2H) may for example increase in-vivo half-life or reduce dosage regimens, or may provide a compound useful as a standard for characterization of biological samples. Isotopically enriched compounds within formula (I) can be prepared by conventional techniques well known to a person skilled in the art or by processes analogous to those described in the general procedures and examples herein using appropriate isotopically enriched reagents and/or intermediates.

In some embodiments, compounds described herein, or pharmaceutically acceptable salts, isomers, or a mixture thereof, have from 1 to n hydrogen atoms attached to a carbon atom replaced by a deuterium atom or D, in which n is the number of hydrogen atoms in the molecule. As known in the art, the deuterium atom is a non-radioactive isotope of the hydrogen atom. Such compounds can increase resistance to metabolism, and thus can be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” TRENDS PHARMACOL. SCI., 5 (12): 524-527 (1984). Such compounds are synthesized by means well known in the art, for example by employing starting materials in which one or more hydrogen atoms have been replaced by deuterium.

In some embodiments, a compound of the disclosure is a solvate or a hydrate.

In one embodiment the disclosure provides a compound as above for use in therapy.

In a further embodiment the disclosure provides a compound as above for use in the treatment of a disease, disorder or condition, which disease, disorder or condition is responsive of modulation of STAT-6.

In a further embodiment the disclosure provides a compound as above for use in the treatment of autoimmune diseases.

The compounds of the present disclosure may be useful for preventing, treating or ameliorating diseases characterized by Th2-mediated inflammation such as atopic dermatitis, asthma, chronic rhinosinusitis with nasal polyposis, urticaria, rhinitis, cosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors.

In an embodiment the disclosure provides the use of a compound of formula (I) as defined above, in the manufacture of a medicament for the prophylaxis, treatment or amelioration of any of the following diseases characterized by Th2-mediated inflammation such as atopic dermatitis, asthma, chronic rhinosinusitis with nasal polyposis, urticaria, rhinitis, cosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors.

In an embodiment the disclosure provides a method of preventing, treating or ameliorating diseases characterized by Th2-mediated inflammation such as atopic dermatitis, asthma, chronic rhinosinusitis with nasal polyposis, urticaria, rhinitis, cosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors, the method comprising administering to a person suffering from at least one of said diseases an effective amount of one or more compounds according to formula (I), optionally together with a pharmaceutically acceptable carrier or one or more excipients, optionally in combination with other therapeutically active compounds.

In an embodiment the disclosure provides a method of preventing, treating or ameliorating autoimmune diseases, conditions characterized by Th2-mediated inflammation such as atopic dermatitis, asthma, chronic rhinosinusitis with nasal polyposis, urticaria, rhinitis, cosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors the method comprising administering to a person suffering from at least one of said diseases an effective amount of one or more compounds according to formula (I), optionally together with a pharmaceutically acceptable carrier or one or more excipients, optionally in combination with other therapeutically active compounds.

Besides being useful for human treatment, the compounds of the present disclosure may also be useful for veterinary treatment of animals including mammals such as horses, cattle, sheep, pigs, dogs, and cats.

### Pharmaceutical Compositions and Modes of Administration

For use in therapy, compounds of the present disclosure are typically in the form of a pharmaceutical composition. In some embodiments, the disclosure provides a pharmaceutical composition comprising a compound of Formula (I), optionally together with one or more other therapeutically active compound(s), together with a pharmaceutically acceptable excipient, vehicle or carrier(s). In some embodiments, the excipient is “acceptable” in the sense of being compatible with the other ingredients of the composition and not deleterious to the recipient thereof.

In some embodiments, the compound of the disclosure is provided in an amount of 0.0001-99.9% by weight of a formulation.

In the form of a dosage unit, a compound of the disclosure may be administered one or more times a day at appropriate intervals. In one embodiments, a dosage unit of a formulation contains between 0.001 mg and 1000 mg, such as between 0.01 mg and 300 mg of a compound of Formula (I).

A suitable dosage of the compound of the disclosure may depend, inter alia, on the age and condition of the patient, the severity of the disease to be treated and other factors well known to the practising physician. The compound may be administered either orally, parenterally, topically, transdermally or intradermally and other routes according to different dosing schedules, e.g. daily, weekly or with monthly intervals. In some embodiments, a single dose comprising a compound of the disclosure may provide an amount of the compound in the range from 0.001 to 400 mg/kg body weight.

In some embodiments, a compound of the disclosure is provided in combination with one or more therapeutically active compounds. the treatment involves administration of another therapeutically active compound it is recommended to consult Goodman & Gilman's The Pharmacological Basis of Therapeutics, 9th Ed., J. G. Hardman and L. E. Limbird (Eds.), McGraw-Hill 1995, for useful dosages of said compounds.

The administration of a compound of the present disclosure with one or more other active compounds may be either concomitantly or sequentially.

The formulations include e.g. those in a form suitable for oral, rectal, parenteral transdermal, intradermal, ophthalmic, topical, nasal, sublingual or buccal administration.

The formulations may conveniently be presented in dosage unit form and may be prepared by but not restricted to any of the methods well known in the art of pharmacy, e.g. as disclosed in Remington, The Science and Practice of Pharmacy, 21ed ed., 2005. All methods include the step of bringing the active ingredient into association with the carrier, which constitutes one or more accessory ingredients. In general, the formulations are prepared by uniformly and intimately bringing the active ingredient into association with a liquid carrier, semisolid carrier or a finely divided solid carrier or combinations of these, and then, if necessary, shaping the product into the desired formulation.

Formulations of the present disclosure suitable for oral and buccal administration may be in the form of discrete units as capsules, sachets, tablets, chewing gum or lozenges, each containing a predetermined amount of the active ingredient.

A tablet may be made by compressing, moulding or freeze drying the active ingredient optionally with one or more accessory ingredients. Compressed tablets may be prepared by compressing, in a suitable machine, the active ingredient(s) in a free-flowing form; for example with a lubricant; a disintegrating agent or a dispersing agent. Moulded tablets may be made by moulding, in a suitable machine, a mixture of the powdered active ingredient and suitable carrier. Freeze dried tablets may be formed in a freeze-dryer from a solution of the drug substance.

Formulations suitable for parenteral administration conveniently comprise a sterile oily or aqueous preparation of the active ingredients, which is preferably isotonic with the blood of the recipient, e.g. isotonic saline, isotonic glucose solution or buffer solution. Liposomal formulations are also suitable for parenteral administration.

Transdermal formulations may be in the form of a plaster, patch, microneedles, liposomal or nanoparticulate delivery systems or other cutaneous formulations applied to the skin.

Formulations suitable for ophthalmic administration may be in the form of a sterile aqueous preparation of the active ingredients. Liposomal formulations or biodegradable polymer systems may also be used to present the active ingredient for ophthalmic administration.

Formulations suitable for topical, such as dermal, intradermal or ophthalmic administration include liquid or semi-solid preparations, solutions or suspensions.

Formulations suitable for nasal or buccal administration include powder, self-propelling and spray formulations, such as aerosols and atomisers.

In some embodiments, administration can be topical (including transdermal, epidermal, ophthalmic and to mucous membranes including intranasal, vaginal and rectal delivery).

In some embodiments, pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient.

In some embodiments, one or more compounds as disclosed herein or a pharmaceutical composition thereof is formulated for topical administration to the skin or mucosa (e.g., dermally or transdermally).

In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating and non-sensitizing.

All references, including publications, patent applications and patents, cited herein are hereby incorporated by reference in their entirety and to the same extent as if each reference were individually and specifically indicated to be incorporated by reference, regardless of any separately provided incorporation of particular documents made elsewhere herein.

### Combination Therapies

In one embodiment, the compounds disclosed herein may be used in combination with one or more additional therapeutic agents. In some embodiments, a compound of the present disclosure, or a pharmaceutically acceptable salt or stereoisomer thereof, can be combined with the therapeutically effective amount of one or more (e.g., one, two, three, four, one or two, one to three, or one to four) additional therapeutic agents.

In some embodiments, a compound of the disclosure, or a pharmaceutically acceptable salt or stereoisomer thereof, is co-administered with one or more agents useful for the treatment and/or prophylaxis of an inflammatory, and/or dermatologic disease or disorder, such as atopic dermatitis (AD). Non-limiting examples of such agents include topical corticosteroids (TCS) (e.g., desonid, hydrocortisone, fluocinolone, triamcinolone, betamethasone diproprionate), topical calcineurin inhibitors (TCI) (e.g., tacrolimus, pimecrolimus), cyclosporine, methotrexate, mycophenolate mofetil, azathioprine, interferon gamma, phosphodiesterase 4 (PDE4) inhibitor such as crisaborole, JAK inhibitor (e.g., ruxolitinib, upadacitinib, abrocitinib, baricitinib), dupilumab, and anti-IL-13 antibody (e.g., tralokinumab).

In some embodiments, a compound of the disclosure, or a pharmaceutically acceptable salt or stereoisomer thereof, is co-administered with one or more agents useful for the treatment and/or prophylaxis of a dermatologic condition, such as acne. Non-limiting examples of such agents include topical therapies such as benzoyl peroxide, topical retinoids, topical antibiotic, clascoterone, salicylic acid and azelaic acid; and systemic therapies such as doxycycline, minocycline, sarecycline, combined oral contraceptives, spironolactone, and isotretinoin.

In some embodiments, a compound of the disclosure, or a pharmaceutically acceptable salt or stereoisomer thereof, is co-administered with one or more agents useful for the treatment and/or prophylaxis of a dermatologic condition, such as alopecia areata. Non-limiting examples of such agents include topical therapies such as systemic corticosteroids (such as prednisolone), cyclosporine, azathioprine, methotrexate, sulfasalazine, simvastatin/exetimibe, inosiplex, antihistamines (such as fexofenadine), and oral JAK inhibitors (such as ritlecitinib or brepocitinib).

In some embodiments, a compound of the disclosure, or a pharmaceutically acceptable salt or stereoisomer thereof, is co-administered with one or more agents useful for the treatment and/or prophylaxis of a dermatologic condition, such as asthma. Non-limiting examples of such agents include inhaled ICS-formoterol (such as budesonide-formoterol), short-acting beta2 agonists (such as albuterol sulfate), leukotriene receptor antagonists (such as montelukast), immunoglobulin E antibodies (such as omalizumab) and long-acting muscarinic antagonists (such as tiotropium).

In some embodiments, a compound of the disclosure, or a pharmaceutically acceptable salt or stereoisomer thereof, is co-administered with one or more agents useful for the treatment and/or prophylaxis of a dermatologic condition, such as chronic obstructive pulmonary disease (COPD). Non-limiting examples of such agents include short-acting beta2 agonists (such as albuterol sulfate), short-acting muscarinic antagonists (such as aiprtropium), long-acting beta2 agonists (such as olodaterol), and long-acting muscarinic antagonists (such as tiotropium).

In some embodiments, a compound of the disclosure, or a pharmaceutically acceptable salt or stereoisomer thereof, is co-administered with one or more agents useful for the treatment and/or prophylaxis of a dermatologic condition, such as chronic rhinosinusitis with polyps. Non-limiting examples of such agents include intranasal corticosteroid, or biologics such as benralizumab (targets IL-5), dupilumab (targets IL-13), omalizumab (targets IgE).

In some embodiments, a compound of the disclosure, or a pharmaceutically acceptable salt or stereoisomer thereof, is co-administered with one or more agents useful for the treatment and/or prophylaxis of a dermatologic condition, such as contact dermatitis. Non-limiting examples of such agents include topical corticosteroids, topical calcineurin inhibitors (such as pimecrolimus, tacrolimus), topical phosphodiesterase 4 inhibitors, such as crisaborole, systemic immunosuppressants and modulators, such as systemic corticosteroids, methotrexate, azathioprine, mycophenolate mofetil, cyclosporine or dupilumab.

In some embodiments, a compound of the disclosure, or a pharmaceutically acceptable salt or stereoisomer thereof, is co-administered with one or more agents useful for the treatment and/or prophylaxis of a dermatologic condition, such as dermatomyositis. Non-limiting examples of such agents include topical corticosteroids, topical calcineurin inhibitors (such as pimecrolimus, tacrolimus), systemic corticosteroids (such as prednisone), antimalarials (such as hydroxychloroquine, cholorquine, quinacrine), methotrexate, mycophenolate mofetil, intravenous immunoglobulin, rituximab, and JAK inhibitors (such as tofacitinib)

In some embodiments, a compound of the disclosure, or a pharmaceutically acceptable salt or stereoisomer thereof, is co-administered with one or more agents useful for the treatment and/or prophylaxis of a dermatologic condition, such as esophageal eosinophilia. Non-limiting examples of such agents include systemic corticosteroids (such as budesonide, fluticasone, prednisone), topical corticosteroids, and proton pump inhibitors (such as omeprazole, esomeprazole, pantoprazole and lansoprazole).

In some embodiments, a compound of the disclosure, or a pharmaceutically acceptable salt or stereoisomer thereof, is co-administered with one or more agents useful for the treatment and/or prophylaxis of a dermatologic condition, such as psoriasis. Non-limiting examples of such agents include topical treatments such as topical corticosteroids (such as betamethasone dipropionate, clobetasol propionate, desoximetasone, diflorasone diacetate, fluocinonide, flurandrenolide, halobetasol propionate, amcinonide, mometasone furoate, triamcinolone acetonide, fluticasone propionate, hydrocortisone valerate, clocortolone pivalate), topical calcineurin inhibitors (such as pimecrolimus, tacrolimus), topical vitamin D analogues (such as calcipotriene, calcitriol, tacalcitol or mazacalcitol), topical retinoids (such as tazarotene); systemic nonbiologic therapies such as methotrexate, phosphodiesterase 4 inhibitors (such as apremilast), immunosuppressants (such as cyclosporine), oral retinoids (such as acitretin), oral Janus kinase inhibitors (such as tofacitinib), fumaric acid esters (such as dimethyl fumarate), systemic immunosuppressants and antimetabolites (such as hydroxyurca, mycophenolate mofetil, azathioprine, leflunomide, tacrolimus and thioguanine); and biologic therapies such as TNF-a inhibitors (such as etanercept, infliximab, adalimumab, certolizumab), IL-12/IL-23 inhibitors (such as ustekinumab), IL-17 inhibitors (such as secukinumab, ixckizumab, brodalumab), and IL-23 inhibitors (such as guselkumab, tildrakizumab, risankizumab).

In some embodiments, a compound of the disclosure, or a pharmaceutically acceptable salt or stereoisomer thereof, is co-administered with one or more agents useful for the treatment and/or prophylaxis of a dermatologic condition, such as scleroderma. Non-limiting examples of such agents include immunosuppressive treatments (such as methotrexate, mycophenolate mofetil, cyclophosphamide, tocilizumab, and rituximab), and autologous haematopoictic stem cell transplantation.

In some embodiments, a compound of the disclosure, or a pharmaceutically acceptable salt or stereoisomer thereof, is co-administered with one or more agents useful for the treatment and/or prophylaxis of a dermatologic condition, such as vitiligo. Non-limiting examples of such agents include topical treatments such as topical corticosteroids, topical calcineurin inhibitors (such as pimecrolimus, tacrolimus), topical vitamin D analogues (such as calcipotriene); and systemic therapies such as oral corticosteroids (such as betamethasone).

In some embodiments, an additional therapeutic agent includes one or more of 608, 610, 611, clindamycin phosphate+benzoyl peroxide, 101BHG-D01, 1-H-11, 4P-022, 5-OXO-ETE receptor antagonists, 9MW-1911, AB-1000, AB-101a, abatacept, ABBV-712, ABCL-575, Ab-IPL-IL-17, ABM-125, abrocitinib, ABY-035/AFO2, ABY-062, AC-201, ACE-1334, acitretin, aclidinium bromide, aclidinium bromide+formoterol fumarate, acumapimod, AD-17002, adakitug, adalimumab, adapalene, adapalene+benzoyl peroxide, adapalene+clindamycin hydrochloride, adapalene+clindamycin phosphate, aderamastat, Adi. ADi-100. Adipocell, adipose tissue-derived mesenchymal stem cell-derived exosomes, adipose-derived stem cell therapy. AD-MSC-CM. ADSTEM, ADX-246, aerosolized hydroxychloroquine, afamelanotide, AJ-101, AJ-303, AK-101, AK-119, AKP-08, albuterol sulfate, ALD-R⁴⁹¹, alefacept, Allergovac depot, allogeneic adipose-derived mesenchymal stem cell therapy, allogeneic adult pluripotent stem cells, allogeneic mesenchymal stem cell therapy, allogeneic UC-MSC therapy, allogeneic umbilical cord mesenchymal stem cell therapy. AlloRx, alprazolam. AM-1476, ambroxol hydrochloride, AMG-0101. Amilo-5MER, aminolevulinic acid, aminolevulinic acid hydrochloride, aminopterin, amlitelimab, AMTX-100, AMTX-100 CF, Anapsos, ANB-032, ANB-101, anifrolumab, anti-CD19 CAR T cell therapy, anti-CD7 CAR T-cell therapy, anti-EMAP II fully humanized antibodies, anti-IL-4/IL-13 vaccine, anti-P2×7 monoclonal antibody humanized, anti-PAR2 therapeutics, antroquinonol, APD-588, APG-222, APG-777, APG-808, APG-990, APGT-001, APIRx-1603, apremilast, aprepitant, APT-101, AQ-001S, AQ-280, AR-100DP1, AR-110, arformoterol, ARG-201, ARGX-118, ARN-4079, ARO-MUC5AC, ARO-RAGE, ARO-TSLP, ARQ-234, arsenic trioxide, ARTS-011, AS-012, asengeprast, asivatrep, ASN-008, astegolimab, AT-004, AT-005, AT-0287, AT-193, ATB-1606, ATI-2138, ATL-105, ATR-006, ATR-01, ATTO-002, ATTO-1310, atuliflapon, AUR-101, auremolimab, autologous leukocyte cell therapy, avenciguat, AVI-3307, AVID-200, AVX-001, AWEPO-003, AX-158, AX-202, AZD-0284, AZD-0449, AZD-8630, azclaic acid, azclastine, azithromycin, B-244, Bacmune, bambuterol, baricitinib, barzolvolimab, BAT-6026, bazlitoran, BB-1511, BBACN, BBI-03, BBI-6000, BCG polysaccharide+nucleic acid, BC₁-332, beclometasone dipropionate+formoterol fumarate, beclomethasone dipropionate, beclomethasone dipropionate+formoterol fumarate+glycopyrronium bromide, bedoradrine, begelomab, belimumab, belumosudil, bempikibart, bencycloquidium bromide, benralizumab, benzoyl peroxide, benzoyl peroxide+tretinoin, berdazimer sodium, bermekimab, bersiporocin dihydrochloride, bertilimumab, betamethasone, betamethasone dipropionate, betamethasone valerate, bexotegrast, BFP-002, BFP-102, BGB-23339, BI-1291583, BI-1323495, BI-765250, bilastine, bimekizumab, bimiralisib, BIO-11006 Inhalation Solution, BioLexa, BITT-CD4D11, BITT-CD4F10, BLR-200, BLU-808, BMS-986313, BMS-986322, BMS-986326, BMX-010, boningmycin, BOS-475, Bosakitug, bosentan, bovhyaluronidase azoximer, Box-5, BR-201, branebrutinib, BRE-AD01, brensocatib, brentuximab vedotin, brepocitinib, brilacidin, brilaroxazine hydrochloride, briquilimab, brodalumab, BSI-056T, BSI-502, BTX-1204, BTX-1308, BTX-1503, budesonide, budesonide+arformoterol, budesonide+formoterol, budesonide+formoterol fumarate, budesonide+procaterol hydrochloride, budesonide+salbutamol, budesonide+salmeterol, buloxibutid, BV-200 series, BVX-20, BZ-371, BZ-371B, C₄X-6746, C-867, CABA-201, CAL-4, calcipotriol, calcipotriol+betamethasone, calcipotriol+betamethasone dipropionate, calcipotriol+cortisone, calcitriol, CALY-002, camoteskimab, CAN-10, cannabidiol, cannabidiol+dronabinol, cannabinoid CB2 receptor agonist antibody, carbon dioxide+perfluorooctyl bromide, cavosonstat, CB-06-01, CB2 receptor agonists, CB5138-3, CC-90006, CC-92252, CC₁-15106, CCX-624, CD19-CAR-DNT, CEE-321, cendakimab, certolizumab pegol, CG-459, Chan1 lergen, CHF-6333, CHF-6366, CHF-6550, ciclesonide, ciclosporin, ciprofloxacin hydrochloride, CIT-013, CJRB-402, CKBA, clascoterone, CLBS-03, clindamycin, clindamycin phosphate+benzoyl peroxide, clindamycin phosphate+tretinoin, clobetasol propionate, clobetasol propionate+tretinoin, CM-101, CM-326, CMK-389, CMR-316, CMS-D001, ColiFin, COPD vaccine, cord blood derived stem cells, corticotropin, COYA-204, CPL-409116, crisaborole, CS-12192, CS-32582, CS-43001, CSJ-117, CSPCHA-115, CT-05, CT-303, CT-P55, CTX-101, CTXT-102, cudetaxestat sodium, CUR-N399, Cutaquig, CVXL-0074, CXF-11, CXG-86, CXG-87, cyproterone acetate+ethinyl estradiol, D-2570, D4-103-01, D4-103-02, D4-103-03, D4-103-04, daniluromer, dapansutrile, dapsone, daridorexant hydrochloride, daxdilimab, dazukibart, DB-007-4, DBI-001, DBM-1152A, DC-806, DC-853, deflazacort, delgocitinib, denifanstat, depemokimab, dersimelagon, desloratadine, desogestrel+ethinylestradiol, desonide, deucravacitinib, deuruxolitinib phosphate, dexamethasone sodium phosphate, dexpramipexole, difamilast, dimethyl fumarate, dimethyl fumarate+ethyl hydrogen fumarate calcium+ethyl hydrogen fumarate magnesium+ethyl hydrogen fumarate zinc, diroleuton, dithranol cream, divozilimab, DLQ-02, DLX-105, DLX-2323, DMT-210, DMT-310, DMX-700, DMXD-011, DNX-114, doxofylline, doxofylline (bronchiectasis), Alitair Pharmaceuticals, doxycycline hyclate, doxycycline hyclate (delayed release), Mayne, doxycycline hyclate (easy-to-swallow, acne, bacterial infection), Aqua Pharmaceuticals, DPT-0218, drospirenone+ethinylestradiol, dual alpha-V/beta-1 and alpha-5/beta-1 integrin inhibitors, dual AMCase/CHIT1 inhibitors, dual anti-CD19/anti-BAFF CAR T-cell therapy, dual JAK3/TEC inhibitor, dupilumab, dust mite vaccine, DW-2008S, DYV-024, DZ-2002, EB-005, EB-06, EBI-H, cblasakimab, cfzofitimod, EI-001, clapegademasc, clarekibep, emedastine, empasiprubart, ENA-002, ENB-109, endonuclease modulators, ENERGI-F708, enpatoran, ensifentrine, ensifentrine+glycopyrrolate, EP-104-GI, EP-262, epeleuton, Epi-13, cpinastine hydrochloride, epinephrine, EpiTight, EPM-301, EQ-101, erdosteine, erlotinib, ESK-001, etanercept, EtanerRel, ETD-001, ETH-47, ctrasimod, ctrinabdione, EVX-B4, EYD-001, F-200, F-528, factor D inhibitor, farudodstat, FB-102, FB-401, FB-704A, FB-825, FB-918, FCR-001, FCX-013, fevipiprant, filgotinib maleate, fipaxalparant, flunisolide, fluocinonide, fluticasone, fluticasone+formoterol, fluticasone furoate, fluticasone furoate+umeclidinium+vilanterol, fluticasone furoate+vilanterol trifenatate, fluticasone propionate, fluticasone propionate+formoterol fumarate, fluticasone propionate+salbutamol sulfate, fluticasone propionate+salmeterol, fluticasone propionate+salmeterol xinafoate, formoterol, formoterol fumarate, formoterol fumarate+fluticasone propionate, formoterol fumarate+glycopyrronium bromide, FPP-003, FPP-005, froniglutide, FRTX-02, FTC-001, FWB-1313, FZ-007, FZJ-003, GABAA receptor agonists, Gamunex, GB-001, GB-0895, GD-134, GD-iExo-001, gefurulimab, GEN-501, GL-7190, GLPG-3667, glutathione+ascorbic acid+bicarbonate, glycopyrrolate+formoterol fumarate+budesonide, glycopyrronium+formoterol fumarate+fluticasone propionate, glycopyrronium+vilanterol, glycopyrronium bromide, glycopyrronium bromide+indacaterol malcate, GMDP, GM-XANTHO, GN-037, GNKS-356, GNR-068, GPCR antagonists, GR-010, GR-1501, GR-1802, GR-2002, GR-2301, Grastek, GRC-39815, GRT-6015, GSK-1070806, GSK-2831781, GSK-3862995B, GSK-3923868, GT-20029, gumokimab, gusacitinib, guselkumab, GZ-21T, H-018, halobetasol propionate, halobetasol propionate+tazarotene, halogenated xanthene, halometasone, HB00-17, HB-0034, HB-0043, HB-1734, HBM-9001, HBM-9378, HCW-9302, HDM-3010, HECB-1800301, HEMP-001, HI-1640V, histamine human immunoglobulin, Hizentra, HJ-787, HL-231, HLA-open conformer-specific monoclonal antibody, HLK-6002, house dust mite allergen, house dust mites immunotherapy, HP-1901, Hpb glutamate dehydrogenase modulator, HPP-737, HpVac-R¹³, HRG-2005, HRS-9821, HS-10374, HS-401, HT-004, HuL-001, human adipose-derived mesenchymal stem cells, human umbilical cord-derived mesenchymal stem cell therapy, HY-07170702, HY-072808, HY-1770, HY-209, hypericin, hypochlorous acid, HZ-J001, IBI-3002, IBI-356, IBIO-100, IBL-101, icanbelimod, ICP-332, ICP-488, iCP-NI, ifetroban, IFNalpha kinoide, IgE inhibitors, IHL-675A, IL-17 NanoAb, IL-25 targeted therapeutic, IL-4R alpha antagonist, IL-4R^{a} targeted therapeutic, ILB-2107, iloprost, IMB-101, IMG-007, IMG-008, IMG-036, immune globulin intravenous, imsidolimab, IMX-120, IN-A002, inaticabtagene autoleucel, INCB-054707, indacaterol, indacaterol acetate+glycopyrronium bromide+mometasone furoate, Indamet, inebilizumab, infliximab, Integrin alpha-2/beta-1 inhibitor, Integrin alpha-5/beta-1 inhibitor, Interleukin IL-17A inhibitor, INV-007, INV-103, INV-17, IPG-1094, IPG-7236, ipratropium+fenoterol, ipratropium bromide, ipratropium bromide+salbutamol sulfate, IR-444, IRL-201104, IRX-4204, isotretinoin, itepekimab, itolizumab, ivacaftor, ivarmacitinib sulfate, ivermectin, ixekizumab, izokibep, JadiCell therapy, JAK inhibitors, JAK-989, jaktinib dihydrochloride monohydrate, jaktinib hydrochloride, JK-0001, JK-0002, JNJ-1459, JNJ-2113, JNJ-3534, JNJ-67484703, JRF-106, JRF-401, JRP-878, JS-005, JTE-051, JTE-451, JW-1601, JW-202232, JW-2202, JYB-1904, JYP-0061, JYP-0066, K-1032, KB-5XX, KBL-693, KBL-697, KI-696, KINE-201, KITCL-27, KN-002, KP-470, KT-294, KT-474, KT-621, KX-826, KYV-101, L-608, LABA+LAMA therapy, Langopept, larsucosterol, LAS-200019, LBG-1600M, LCK inhibitor, lebrikizumab, lepzacitinib, levalbuterol, levalbuterol hydrochloride, levonorgestrel+ethinylestradiol, LG-283, LGM-1506, LGM-2605, LH-8, LIT-00505, lithium succinate, LMY-920, LNK-01001, LNK-01004, LNP-1955, LNR-653,1, londamocitinib, long acting beta agonist/long acting muscarinic agonist, long-acting aerosolized peptide-based therapy, lonodelestat acetate, Ip-003, LP-0200, LQ-036, LQ-041, LQ-043, LR-19019, LR-20016, LT-002-158, lucinactant, lunsckimig, LUT-014, LW-104, LY-3509754, LY-3872386, LY-3972406, LYS-006, lysophospholipase inhibitor, LZM-012, M-119102, M3 muscarinic receptor antagonists, M-605110, M-610101, manfidokimab, masitinib, MAX-40070, maxacalcitol, maxacalcitol+betamethasone, MCM-001, MDI-1228, MDNA-413, MDPK-67b, ME-3183, Melgain, mepolizumab, mesalazine, Mesenchymal stem/stromal cell therapy, mesenchymoangioblast-derived mesenchymal stem cell therapy, metenkefalin acetate+tridecactide acetate, methotrexate, methyl aminolevulinate hydrochloride, methylprednisolone suleptanate, MG-01, MG-K10, MG-S-2525, MGY-1838, MG-ZG122, MH-004, MH-080, minocycline, minocycline+adapalene, minocycline hydrochloride, MIT-001, mitiperstat, Mitizax, MM-09, mometasone, mometasone+formoterol, mometasone furoate, mometasone furoate+indacaterol acetate, mon1 unabant, montelukast, montelukast sodium, montelukast sodium+levocetirizine dihydrochloride, mosedipimod, mouse monoclonal antibody against human interleukin-8, MP-1032, MSB-01, MSB-03, MSB-3163, MSM-605, MT-5562, MTC-896, mucosa-associated lymphoid tissue lymphoma translocation protein 1 inhibitors, mufemilast, MufroSyn, mugwort pollen allergen vaccine, muscarinic M3 receptor antagonist, MYJ-1633, nacystelyn, nadifloxacin, nadolol, nalfurafine, NBL-012, NCP-111, NCP-112, ND-003, NDX-3315, NDX-3324, nedocromil, nemolizumab, netakimab, nibrozetone, niclosamide, NIK inhibitors, nitric oxide, nitroglycerin, NLP-91, NM26-2198, nomacopan, norethindrone acetate+ethinylestradiol, noscapine/noscapine analogs, NP-339, nrf2 activator, NS-402, NTR-441, NVS-451, NX-73, OATD-01, OB-756, obefazimod, OC-701, OCR-4715, Octagam 10%, olodaterol, olodaterol hydrochloride+tiotropium bromide monohydrate 1, olopatadine, OLX-103, OM-001, omalizumab, omiganan pentahydrochloride, omilancor, OMN-71, ONO-4685, OP-2101, opinercept, OpSCF, ordesekimab, ORI-001, orismilast, ORKA-001, ORKA-002, orticumab, ozagrel hydrochloride, ozenoxacin, PA-9159, paridiprubart, PBF-680, PBI-100, PC-114, PDC-APB, PDE4 inhibitor, pegtarazimod, pemirolast, peresolimab, PF-07264660, PF-07275315, PG-011, PG-102, Viromed, phimelanotide, PHP-1212, PI3K-delta inhibitor, picankibart, piclidenoson, pimecrolimus, PIPE-791, pirfenidone, pitavastatin, PKC theta inhibitors, PLM-301, PNV-5032, POLB-002, ponesimod, potassium dobesilate, PR-023, pran1 ukast, pran1 ukast hydrate, PRCL-02, PrEP-001, prostaglandin D2 synthase inhibitors, Prozumab, PRP-PBMC autologous cellular therapy, PS-35, psoriasis therapeutics, PT-101, P-TET, PUL-042, PUR-0110, PUR-1800, PX-128, PX-130, PZ-07/2024, Q-1804, Q-301, QBKPN, QLM-3003, QN-02, QP-CO1, QRX-008, quisovalimab, QX-002-N, QX-004-N, QX-005-N, QX-007-N, QX-008-N, QX-009-N, QX-010-N, QY-101, QY-201, QY-211, R-187, R-552, rademikibart, rare phytocannabinoids, ravulizumab, RAY-121, RB-1000, RBM-009, RBN-012759, RBO-0987, RC-1416, RCD-405, recombinant midismase, reformulated calcipotriol+betamethasone, REGEND-001, REGN-1908-1909, remetinostat, remibrutinib, renzapride, repirinast, repurposed aldesleukin, Repurposed azeliragon, repurposed lenabasum, reslizumab, RESP-1000 series, RESP-2000 series, RESP-X, retinoic acid, revefenacin, REX-7117, rezpegaldesleukin, RG-6151, RG-6244, RG-6314, RG-6315, RG-6341, RG-6421, RGRN-305, rilzabrutinib, riociguat, risankizumab, ritlecitinib, rituximab, RLS-1496, RLV-102, rocatin1 imab, roflumilast, ropsacitinib, ROR gamma T inverse agonists, ROR-gamma inverse agonists, rose bengal sodium, RP-3128, RSBT-001, RSS-0393, R-TPR-022, rupatadine+montelukast, RUTI, ruxolitinib, RYSW-01, S1P1 agonist, salbutamol, salmeterol, salmeterol xinafoate+fluticasone propionate, SAMiRNA program, SAR-441566, SAR-443726, sarecycline, SB-010, SB-011, SCD-044, SCD-153, SCT-640A, SCT-650-C, SDC-1801, sccukinumab, SEGRA, seletalisib, SEL-K2, selnoflast, seratrodast, SFA-002, SFA-004, SG-100, SGT-510, SH2 domain inhibitor program targeting STAT6, SHR-1703, SHR-1819, SHR-1905, SHR-4597, si-544, SIG-1322, SIG-1451, SIG-1456, SIM-0278, SIM-335, sitaxentan, SKI-O-703, SKL-XYZ, SLS-008, SM-17, small mobile stem cell therapy, SMET-D1, SNC-103, SNG-001, SNG-100, SNK-01, sodium chromoglycate, sodium pyruvate, sonclokimab, soquelitinib, sovleplenib, spesolimab, SPL84-23, SSGJ-621, SSS-07, ST-1830, stapokibart, STAR-0310, STAT3 inhibitor, STMC-103H, STS-01, STSA-1201, SUDO-286, SUL-238, SuperMApo, suplatast tosilate, SYHX-1901, SYX-5219, T-517, tacalcitol, tacrolimus, TAF-001, tagraxofusp, TAGX-0003, TAKC-02, tanimilast, TAP-1502, TAP-1503, tapinarof, Tavo-101, tazarotenc, tazarotene+betamethasone dipropionate, tazarotene+clindamycin, TD-8236, TDM-180935, TDM-Atop01, TDM-Psor01, TDM-Scar01, telazorlimab, Tempol, temtokibart, teprotumumab, TER-101, terbutalin, terguride, tesnatilimab, TEV-48574, TEV-53275, tezepelumab, TFF-HMW-HA, Thalassophryne nattereri peptide, THB-001, theophylline, THOR-809, TI-520, TI-620, tibulizumab, tildrakizumab, timolumab, tiotropium, tiotropium bromide, tipelukast, tirbanibulin, TLL-018, TLY-012, TO-210, tofacitinib, tofacitinib +fingolimod, tofacitinib citrate, tonabacase, TOP-N44, TOP-N53, torudokimab, tosufloxacin, tozorakimab, TP-317, TQC-2731, TQC-2938, TQC-3564, TQC-3721, TQC-3927, TQH-2722, TQH-2929, TQH-3906, TQH-3910, trabikibart, tralokinumab, tranilast, transcription factor pathway inhibitor, tregalizumab, treprostinil, treprostinil diolamine, tretinoin, tretinoin+benzoyl peroxide, trifarotene, TRIV-509, TRN-157, TRPA1 antagonists, TS-0001, TT-01, TT-01688, tulinercept, tulobuterol, TVB-3567, UA-021, UB-221, UCB-1381, UCB-9741, ucenprubart, UHE-101, UHE-105, UI-009, UI-010, UI-031, UI-033, UI-034, ulobetasol, umbilical cord blood-derived stem cell therapy, UMC119-06, umeclidinium bromide, umeclidinium bromide+vilanterol trifenatate, upadacitinib, USP-4 inhibitors, ustekinumab, UTAA-09, VALERGEN-DS, vamorolone, vapendavir, vardenafil, VB-1953, VC-005, VDAA, VDAD, VDJ-006, VEGFR targeted DK4/10, venanprubart, VENT-03, verekitug, vilanterol+fluticasone furoate+glycopyrronium bromide, vipoglanstat hydrogensulfate, VISTA agonist, vixarclimab, VLRX-001, VM-AD, VNLG-152, vonifimod, voriconazole, votucalis, VRN-04, VS-105, VSG-158, VSTM-1 agonist, VT-014, VTH-212, vunakizumab, VYN-201, VYN-202, W16P-0576, WD-890, WM-1R3, WNT inhibitor, WNT pathway agonist antibodies, wnt pathway stimulator, WXFL-10203614, WXSH-0150, XCUR-17, XKH-001, XmAb-564, XT-0528, XZ,700, YH-25487, YH-35324, YKRH-00020, YR-001, Yso3, zabedosertib, zafirlukast, zasocitinib, ZB-168, Zemaira, ZeP-3, zetomipzomib, zevaquenabant, ZHB-107-108, zibotentan, zileuton, zirconium zr 89 crefmirlimab berdoxam, ZL-1102, ZPL-521, and/or bi-specific antibodies targeting one or more targets referenced herein.

In some embodiments a compound of the disclosure provided herein, or pharmaceutically acceptable salt or stereoisomer thereof, is administered with one or more therapeutic agents selected from a PPARd inhibitor, IRAK4 inhibitor, TPL2 inhibitor, α4β7 inhibitor, BTLA agonist, PD1 agonist, or an FXR agonist.

In some embodiments a compound of the disclosure provided herein, or pharmaceutically acceptable salt or stereoisomer thereof, is administered with one or more therapeutic agents selected from seladelpar. edecesertib, tilpisertib fosmecarbil. GS-1427. GS-0272. GS-0151, or cilofexor.

The benefit of combination may be increased efficacy and/or reduced side effects for a component as the dose of that component may be adjusted down to reduce its side effects while benefiting from its efficacy augmented by the efficacy of the compound of the present disclosure.

In some embodiments, the additional therapeutic agent includes an agent useful for modulating, treating, or preventing inflammation, such as a 4-1BB ligand, 5-Alpha-reductase inhibitor, 5-HT 1a receptor antagonist, 5-HT 1a receptor partial agonist, 5-HT 2a receptor antagonist, 5-HT 2a receptor partial agonist, 5-HT 2b receptor antagonist, 5-HT 3 receptor antagonist, 5-HT 4 receptor agonist, 5-HT 6 receptor antagonist, 5-HT 7 receptor antagonist, 5-Lipoxygenase activating protein inhibitor, 5-Lipoxygenase inhibitor, Accessory gene regulator A inhibitor, Acetaldehyde dehydrogenase modulator, Acetylcholine receptor agonist, Acetylcholine receptor antagonist, Acetylcholinesterase inhibitor, Acidic mammalian chitinase inhibitor, ACTH receptor agonist, Activity-dependent neuroprotector modulator, ADAM-33 inhibitor, ADAM-9 inhibitor, Adenosine A1 receptor antagonist, Adenosine A1 receptor modulator, Adenosine A2b receptor antagonist, Adenosine A3 receptor agonist, Adenosine A3 receptor antagonist, Adenosine deaminase stimulator, Adenosylhomocysteinase inhibitor, Adenylate cyclase stimulator, Adrenergic receptor agonist, Adrenocorticotrophic hormone ligand, Advanced glycosylation product receptor antagonist, AGER gene inhibitor, AIMP multisynthetase complex protein 1 inhibitor, Albumin antagonist, Alcohol dehydrogenase 5 inhibitor, Aldose reductase inhibitor, Alk-5 protein kinase inhibitor, Alpha 1 antitrypsin modulator, Alpha 1 antitrypsin stimulator, Alpha 1 proteinase inhibitor, Alpha 2 adrenoceptor agonist, Amiloride sensitive sodium channel inhibitor, AMP activated protein kinase alpha 2 stimulator, AMP activated protein kinase stimulator, Amyloid protein deposition inhibitor, Androgen receptor antagonist, Angiotensin II AT-1 receptor antagonist, Angiotensin II AT-2 receptor agonist, Anoctamin 1 stimulator, Aortic smooth muscle actin inhibitor, AP1 transcription factor modulator, Apelin receptor agonist, Apolipoprotein A antagonist, Apolipoprotein A5 stimulator, Apolipoprotein B modulator, Apolipoprotein E modulator, Apoptosis regulator Bcl X inhibitor, Apoptosis regulator Bcl w inhibitor, APRIL receptor modulator, Aryl hydrocarbon receptor agonist, Aryl hydrocarbon receptor modulator, B and T lymphocyte attenuator stimulator, B-lymphocyte antigen CD19 inhibitor, B-lymphocyte antigen CD19 modulator, B-lymphocyte antigen CD20 inhibitor, B-lymphocyte stimulator ligand inhibitor, B-lymphocyte stimulator ligand modulator, Bcl-2 protein inhibitor, Beta 1 adrenoceptor antagonist, Beta 2 adrenoceptor agonist, Beta 2 adrenoceptor antagonist, Beta 2 adrenoceptor modulator, Beta adrenoceptor agonist, Beta amyloid antagonist, Beta-catenin inhibitor, Beta-catenin modulator, Bifunctional aminoacyl tRNA synthetase inhibitor, BMP10 gene inhibitor, BMP15 gene inhibitor, Bone marrow proteoglycan modulator, Botulinum toxin A stimulator, Bromodomain containing protein 1 inhibitor, Bromodomain containing protein inhibitor, Btk tyrosine kinase inhibitor, C-myc binding protein inhibitor, C-type lectin domain protein 4C inhibitor, Ca2+release activated Ca2+channel 1 inhibitor, Calcineurin inhibitor, Calcium channel inhibitor, Cannabinoid CB1 receptor antagonist, Cannabinoid CB1 receptor inverse agonist, Cannabinoid CB2 receptor agonist, Cannabinoid CB2 receptor modulator, Cannabinoid receptor agonist, Cannabinoid receptor antagonist, Cannabinoid receptor modulator, Catalase stimulator, CCL26 gene inhibitor, CCR3 chemokine modulator, CCR5 chemokine antagonist, CCR6 chemokine antagonist, CCR8 chemokine antagonist, CD11b antagonist, CD122 agonist, CD122 modulator, CD19 modulator, CD2 antagonist, CD223 modulator, CD3 modulator, CD30 modulator, CD4 antagonist, CD40 ligand receptor antagonist, CD47 antagonist, CDw123 modulator, Cell adhesion molecule inhibitor, Cell surface glycoprotein CD200R agonist, Cell surface glycoprotein MUC18 inhibitor, CFTR modulator, CFTR stimulator, Chaperonin stimulator, Chemokine receptor antagonist, Chitinase inhibitor, Collagen I agonist, Collagen I antagonist, Collagen modulator, Collagen VII antagonist, Complement C₁q subcomponent inhibitor, Complement C₁s subcomponent inhibitor, Complement C₅ factor inhibitor, Complement factor C₂ inhibitor, Complement factor D inhibitor, COVID19 spike glycoprotein modulator, CSF-1 antagonist, CXC10 chemokine ligand inhibitor, CXCR2 chemokine antagonist, cyclic GMP AMP synthase inhibitor, Cyclooxygenase inhibitor, Cytokine receptor agonist, Cytokine receptor antagonist, Cytokine receptor common beta chain inhibitor, Cytoplasmic protein NCK inhibitor, Cytosolic phospholipase A2 inhibitor, Cytotoxic T-lymphocyte protein-4 stimulator, Deoxyribonuclease gamma stimulator, DHFR inhibitor, Diacylglycerol O acyltransferase 1 inhibitor, Dihydroorotate dehydrogenase inhibitor, Dipeptidyl peptidase I inhibitor, Dipeptidyl peptidase IV inhibitor, DNA gyrase inhibitor, DNA methyltransferase inhibitor, Dopamine D2 receptor partial agonist, Dopamine D3 receptor agonist, Dopamine D3 receptor partial agonist, Dopamine D4 receptor partial agonist, DYRK-1 alpha protein kinase inhibitor, Ectonucleotide pyrophosphatase-PDE-2 inhibitor, EGF like module receptor 1 antagonist, EGFR family tyrosine kinase receptor inhibitor, Elastase inhibitor, Endonuclease modulator, Endostatin modulator, Endothelin ET-A receptor antagonist, Endothelin ET-B receptor antagonist, Enolase 1 inhibitor, Eosinophil peroxidase inhibitor, Eotaxin 2 ligand inhibitor, Eotaxin ligand inhibitor, EP4 prostanoid receptor antagonist, Epidermal growth factor receptor antagonist, Epidermal growth factor receptor modulator, Estradiol agonist, Estrogen receptor agonist, Extracellular signal related kinase-2 inhibitor, Facilitated glucose transporter-1 modulator, Fatty acid synthase inhibitor, FGF receptor antagonist, FGF-2 ligand, FGF-4 ligand, FGF3 receptor antagonist, Filaggrin stimulator, Flt3 tyrosine kinase inhibitor, FMLP related receptor I agonist, FMLP related receptor II agonist, Free fatty acid receptor 2 agonist, Frec fatty acid receptor 3 agonist, Fyn tyrosine kinase inhibitor, FXR agonist, G-protein coupled bile acid receptor 1 agonist, G-protein coupled receptor-44 antagonist, G-protein coupled receptor-44 modulator, GABA A receptor agonist, GABA A receptor alpha-2 subunit modulator, GABA A receptor alpha-3 subunit modulator, GABA A receptor alpha-5 subunit modulator, Galectin-10 modulator, GATA 3 transcription factor inhibitor, Glucagon-like peptide 1 receptor agonist, Glucocorticoid receptor agonist, Glutamate dehydrogenase modulator, Glutamate receptor modulator, Glutathione dependent PGD synthase inhibitor, Glutathione independent PGD synthase inhibitor, Glutathione reductase inhibitor, GroEL protein 2 inhibitor, Guanylate cyclase stimulator, H+ K+ ATPase inhibitor, Heat shock protein inhibitor, Heme oxygenase 1 modulator, Heparin agonist, High mobility group protein B1 inhibitor, Histamine H1 receptor antagonist, Histamine H4 receptor antagonist, Histamine receptor antagonist, Histone deacetylase-1 inhibitor, Histone deacetylase-2 inhibitor, Histone deacetylase-2 stimulator, Histone deacetylase-3 inhibitor, Histone deacetylase-6 inhibitor, Histone H2A modulator, Histone H4 modulator, HMG CoA reductase inhibitor, Hsp 90 inhibitor, Hsp70 binding protein 1 inhibitor, Hyaluronidase stimulator, Hypoxia inducible factor stimulator, I-kappa B kinase beta inhibitor, I-kappa B kinase epsilon inhibitor, IgG receptor FcRn large subunit p51 modulator, IL-1 receptor accessory protein inhibitor, IL-1 receptor antagonist, IL-10 receptor agonist, IL-10 receptor antagonist, IL-12 receptor antagonist, IL-13 receptor antagonist, IL-13 receptor modulator, IL-15 receptor antagonist, IL-17 antagonist, IL-18 antagonist, IL-2 receptor alpha subunit stimulator, IL-2 receptor antagonist, IL-2 receptor modulator, IL-22 antagonist, IL-23 antagonist, IL-3 receptor modulator, IL-4 receptor antagonist, IL-4 receptor modulator, IL-5 receptor antagonist, IL-6 receptor antagonist, IL-7 receptor antagonist, IL-8 receptor antagonist, IL17RA gene inhibitor, IL2 gene stimulator, Immunoglobulin E antagonist, Immunoglobulin E modulator, Immunoglobulin G agonist, Immunoglobulin G1 modulator, Immunoglobulin agonist, Immunoglobulin kappa modulator, Immunoglobulin modulator, Inducible nitric oxide synthase inhibitor, Insulin receptor substrate-1 inhibitor, Insulin-like growth factor 1 receptor antagonist, Integrin alpha-2/beta-1 antagonist, Integrin alpha-4/beta-1 antagonist, Integrin alpha-4/beta-7 antagonist, Integrin alpha-5/beta-1 antagonist, Integrin alpha-5/beta-3 modulator, Integrin alpha-V/beta-1 antagonist, Integrin beta 1 binding protein modulator, Interferon alpha 14 ligand, Interferon alpha ligand inhibitor, Interferon beta ligand, Interferon beta ligand inhibitor, Interferon gamma ligand inhibitor, Interferon gamma receptor antagonist, Interferon type I receptor antagonist, Interleukin 1 delta ligand inhibitor, Interleukin 1 like receptor (IL33R) antagonist, Interleukin 1 like receptor 1 modulator, Interleukin 1 like receptor 2 inhibitor, Interleukin 13 ligand inhibitor, Interleukin 13 receptor alpha 1 antagonist, Interleukin 15 ligand inhibitor, Interleukin 17 ligand inhibitor, Interleukin 17A ligand inhibitor, Interleukin 17A ligand modulator, Interleukin 17E ligand inhibitor, Interleukin 17E ligand modulator, Interleukin 17F ligand inhibitor, Interleukin 17F ligand modulator, Interleukin 18 ligand inhibitor, Interleukin 23A inhibitor, Interleukin 31 ligand inhibitor, Interleukin 31 ligand modulator, Interleukin 33 ligand inhibitor, Interleukin 33 ligand modulator, Interleukin receptor 17A antagonist, Interleukin receptor 17B antagonist, Interleukin-1 alpha ligand inhibitor, Interleukin-1 beta ligand modulator, Interleukin-1 ligand inhibitor, Interleukin-2 ligand, Interleukin-2 ligand inhibitor, Interleukin-31 receptor modulator, Interleukin-4 ligand inhibitor, Interleukin-5 ligand inhibitor, Interleukin-6 ligand inhibitor, Interleukin-8 ligand inhibitor, Interleukin-9 ligand inhibitor, IRAK-4 protein kinase inhibitor, IRAK-4 protein kinase degrader, Itk tyrosine kinase inhibitor, JAK tyrosine kinase inhibitor, Jak1 tyrosine kinase inhibitor, Jak2 tyrosine kinase inhibitor, Jak3 tyrosine kinase inhibitor, Jun N terminal kinase inhibitor, Kallikrein 2 inhibitor, Kallikrein 5 modulator, Kallikrein 7 inhibitor, Kallikrein 7 modulator, Kallikrein inhibitor, KCNA voltage-gated potassium channel-3 inhibitor, Kelch like ECH associated protein 1 inhibitor, Kelch like ECH associated protein 1 modulator, Kit tyrosine kinase inhibitor, LanC like protein 2 stimulator, Lanosterol-14 demethylase inhibitor, Lck tyrosine kinase inhibitor, Lectin mannose binding protein inhibitor, Leukocyte Ig like receptor A4 modulator, Leukocyte elastase inhibitor, Leukotriene A4 hydrolase inhibitor, Leukotriene BLT receptor antagonist, Leukotriene C₄ antagonist, Leukotriene C₄ synthase inhibitor, Leukotriene D4 agonist, Leukotriene D4 antagonist, Leukotriene E4 antagonist, Leukotriene receptor antagonist, Liver X receptor agonist, LOXL2 gene inhibitor, Lung surfactant associated protein D stimulator, Lyn tyrosine kinase inhibitor, Lysophosphatidate-1 receptor antagonist, Lysophospholipase inhibitor, Macrophage migration inhibitory factor inhibitor, Major allergen I polypeptide chain 2 inhibitor, Major allergen inhibitor, MALT protein 1 inhibitor, Mannan-binding lectin serine protease inhibitor, MAP kinase modulator, MAPKAPK2 inhibitor, MARCKS protein inhibitor, Mas-related G-protein receptor X² antagonist, Mas-related G-protein receptor X² inhibitor, MEK protein kinase inhibitor, MEK-1 protein kinase inhibitor, Melanocortin MC1 receptor agonist, Melanocortin MC5 receptor antagonist, Melanocortin receptor agonist, Melanocyte stimulating hormone ligand, Membrane copper amine oxidase inhibitor, Metalloprotease-12 inhibitor, MEX3B gene inhibitor, Mineralocorticoid receptor antagonist, MIP 3 alpha ligand inhibitor, Mite allergen modulator, Mitochondrial 10 kDa heat shock protein stimulator, MKL myocardin like protein inhibitor, MMP1 gene stimulator, MNK protein kinase inhibitor, Monocyte chemotactic protein 1 ligand inhibitor, MS4A2 gene modulator, mTOR complex 1 inhibitor, mTOR complex 2 inhibitor, MUC5AC gene inhibitor, Muscarinic M1 receptor antagonist, Muscarinic M2 receptor antagonist, Muscarinic M3 receptor antagonist, Muscarinic M4 receptor antagonist, Muscarinic M5 receptor antagonist, Muscarinic receptor agonist, Muscarinic receptor antagonist, Muscarinic receptor modulator, Myeloperoxidase inhibitor, Myosin heavy chain inhibitor, NACHT LRR PYD domain protein 3 inhibitor, NEDD4 family interacting protein 1 stimulator, Neuropilin 2 modulator, Neutral endopeptidase inhibitor, NFE2L2 gene stimulator, Nicotinic ACh receptor alpha 7 subunit stimulator, Nicotinic acetylcholine receptor agonist, NK1 receptor antagonist, NKG2 D activating NK receptor antagonist, NLR family member X¹ stimulator, Non receptor tyrosine kinase TYK2 antagonist, Nuclear erythroid 2-related factor 1 stimulator, Nuclear erythroid 2-related factor 2 stimulator, Nuclear erythroid 2-related factor inhibitor, Nuclear factor kappa B gene inhibitor, Nuclear factor kappa B inducing kinase inhibitor, Nuclear factor kappa B inhibitor, Nuclear factor kappa B modulator, Oncostatin M receptor subunit beta inhibitor, Opioid growth factor receptor agonist, Opioid receptor delta antagonist, Opioid receptor kappa agonist, Orexin 1 receptor antagonist, Orexin 2 receptor antagonist, Orphan nuclear receptor antagonist, Outer membrane protein modulator, OX-40 receptor agonist, OX-40 receptor antagonist, OX40 ligand inhibitor, OX40 ligand modulator, Oxocicosanoid receptor 1 antagonist, P-Glycoprotein inhibitor, P-selectin glycoprotein ligand-1 inhibitor, P2×2 purinoceptor antagonist, P2×3 purinoceptor antagonist, P2×7 purinoceptor modulator, P2Y⁶ purinoceptor modulator, p38 MAP kinase inhibitor, p53 tumor suppressor protein stimulator, PerV protein type III inhibitor, PDE 3 inhibitor, PDE 4 inhibitor, PDE 4b inhibitor, PDE 4d inhibitor, PDE 5 inhibitor, PDGF receptor antagonist, Peptidase 1 inhibitor, PGD2 antagonist, PGI2 agonist, Phosphatidylinositol 4 kinase beta inhibitor, Phosphoinositide 3-kinase inhibitor, Phosphoinositide-3 kinase delta inhibitor, Phospholipase A2 inhibitor, Phospholipase C inhibitor, PIM-1 protein kinase inhibitor, PIM-2 protein kinase inhibitor, PIM-3 protein kinase inhibitor, Placenta growth factor ligand inhibitor, Plasminogen activator inhibitor 1 inhibitor, Platelet activating factor receptor antagonist, Poly ADP ribose polymerase 14 inhibitor, PPAR gamma agonist, PPAR gene modulator, Progesterone receptor agonist, Programmed cell death ligand 1 modulator, Programmed cell death protein 1 modulator, Programmed cell death protein 1 stimulator, Prostaglandin E synthase inhibitor, Prostaglandin E synthase-1 inhibitor, Protease inhibitor, Protease-activated receptor-2 antagonist, Proteasome beta-8 subunit modulator, Proteasome inhibitor, Protein kinase C theta inhibitor, Protein kinase inhibitor, Protein NOV homolog modulator, Protein tyrosine kinase inhibitor, Protoporphyrinogen oxidase modulator, Pyruvate kinase muscle isozyme stimulator, Raf B protein kinase inhibitor, Ras gene inhibitor, Reactive oxygen species modulator inhibitor, Ret tyrosine kinase receptor inhibitor, Retinoic acid receptor agonist, Retinoic acid receptor antagonist, Retinoic acid receptor gamma agonist, Retinoic acid receptor gamma antagonist, Retinoic acid receptor gamma inverse agonist, Retinoic acid receptor modulator, Retinoid receptor agonist, Retinoid X receptor agonist, Retinoid X receptor modulator, Retinoid Z receptor gamma antagonist, Retinoid Z receptor gamma inverse agonist, Rev protein modulator, Rho associated protein kinase 1 inhibitor, Rho associated protein kinase 2 inhibitor, Ribonuclease P inhibitor, Ribonuclease stimulator, RIP-1 kinase inhibitor, S100 calcium binding protein A4 inhibitor, S100A8 gene inhibitor, S100A9 gene inhibitor, SARS coronavirus 3C protease like inhibitor, Secretory phospholipase A2 receptor antagonist, Serine protease inhibitor, Serine threonine protein kinase TBK1 inhibitor, Serum amyloid A protein modulator, SH2 domain containing protein inhibitor, Sialic acid-binding Ig-like lectin 8 inhibitor, SIRT3 gene stimulator, SMAD inhibitor, SNAI1 transcription factor inhibitor, SOD3 gene stimulator, Sodium channel inhibitor, Sphingosine 1 phosphate phosphatase 1 stimulator, Sphingosine-1-phosphate receptor-1 agonist, Sphingosine-1-phosphate receptor-1 antagonist, Sphingosine-1-phosphate receptor-1 modulator, Sphingosine-1-phosphate receptor-3 modulator, Sphingosine-1-phosphate receptor-4 antagonist, Sphingosine-1-phosphate receptor-4 modulator, Sphingosine-1-phosphate receptor-5 modulator, Sphingosylphosphorylcholine receptor antagonist, Src tyrosine kinase inhibitor, STAT inhibitor, STAT-1 modulator, STAT-3 inhibitor, STAT-5 inhibitor, STAT-6 inhibitor, STAT-6 degrader, Stearoyl CoA desaturase-1 inhibitor, Stress induced secreted protein 1 stimulator, Superoxide dismutase modulator, Superoxide dismutase stimulator, Syk tyrosine kinase inhibitor, Synuclein alpha inhibitor, T cell receptor antagonist, T cell receptor modulator, T cell surface glycoprotein CD28 inhibitor, T-cell antigen CD7 modulator, T-cell differentiation antigen CD6 inhibitor, T-cell surface glycoprotein CD8 inhibitor, T-cell transcription factor NFAT modulator, Tau aggregation inhibitor, Tau deposition inhibitor, Tec tyrosine kinase inhibitor, TGF beta 1 ligand inhibitor, TGF beta 3 ligand inhibitor, TGF beta 3 ligand modulator, TGF beta ligand inhibitor, TGF beta receptor agonist, TGF beta receptor antagonist, TGF-beta activated kinase-1 inhibitor, TGF-beta type II receptor antagonist, TGF-beta type II receptor modulator, TGF-beta type III receptor antagonist, Thromboxane A2 antagonist, Thromboxane synthetase inhibitor, Thymic stromal lymphopoietin ligand, Thymic stromal lymphopoietin ligand inhibitor, Thymic stromal lymphopoietin ligand modulator, Thymic stromal lymphopoietin receptor antagonist, Thymic stromal lymphopoietin receptor modulator, TLR agonist, TLR modulator, TLR-2 agonist, TLR-2 antagonist, TLR-4 antagonist, TLR-6 agonist, TLR-7 antagonist, TLR-8 antagonist, TLR-9 agonist, TLR-9 antagonist, TNF agonist, TNF alpha ligand inhibitor, TNF alpha ligand modulator, TNF antagonist, TNF binding agent, TNF related apoptosis inducing ligand, TNF-like receptor-2 modulator, Tumor necrosis factor 15 ligand inhibitor, Topoisomerase IV inhibitor, TRAIL receptor agonist, Transcription factor inhibitor, Transcription factor modulator, Transthyretin modulator, Trk tyrosine kinase receptor inhibitor, TRP cation channel A1 inhibitor, TRP cation channel A1 modulator, TRP cation channel C₁ inhibitor, TRP cation channel V1 antagonist, TRP cation channel V1 modulator, TRP cation channel V2 modulator, Ts1 protein kinase inhibitor, Tubulin binding agent, Tubulin receptor antagonist, Tumor necrosis factor 13B receptor modulator, Tumor necrosis factor 13C receptor modulator, Tumor necrosis factor 14 ligand inhibitor, Tumor necrosis factor 15 ligand modulator, Tumor necrosis factor ligand inhibitor, Txk tyrosine kinase inhibitor, Tyk2 tyrosine kinase inhibitor, Tyk2 tyrosine kinase modulator, Type I IL-1 receptor antagonist, Type I TNF receptor antagonist, Type II TNF receptor modulator, Tyrosine phosphatase substrate 1 inhibitor, Ubiquitin inhibitor, Ubiquitin ligase modulator, Ubiquitin ligase stimulator, Ubiquitin thioesterase-4 inhibitor, Unspecified GPCR antagonist, Unspecified GPCR modulator, Unspecified ion channel inhibitor, Uteroglobin stimulator, V-set transmembrane domain protein 1 stimulator, Vascular cell adhesion protein 1 antagonist, VEGF ligand inhibitor, VEGF receptor modulator, VEGF-2 receptor modulator, Vimentin inhibitor, Vitamin D3 receptor agonist, Wnt 5A ligand inhibitor, Wnt ligand modulator or YSK-4 protein kinase inhibitor.

### Kits

Provided herein are also kits that include a compound described herein, or a pharmaceutically acceptable salt, tautomer, stereoisomer, mixture of stereoisomers, prodrug, or deuterated analog thereof, and suitable packaging. In one embodiment, a kit further includes instructions for use. In one aspect, a kit includes a compound of Formula I (or any other Formula described herein), or a pharmaceutically acceptable salt, tautomer, stereoisomer, mixture of stereoisomers, prodrug, or deuterated analog thereof, and a label and/or instructions for use of the compounds in the treatment of the indications, including the diseases or conditions, described herein.

Provided herein are also articles of manufacture that include a compound described herein or a pharmaceutically acceptable salt, tautomer, stereoisomer, mixture of stereoisomers, prodrug, or deuterated analog thereof in a suitable container. The container may be a vial, jar, ampoule, preloaded syringe, and intravenous bag.

### EXAMPLES

### Experimental Procedures

Intermediates were obtained sufficiently pure for the next step from the procedures outlined in the specification.

Reactions were performed at room temperature un1 ess stated otherwise. When a compound whose synthesis is described herein was used at a larger scale than its reported synthesis, it is to be implicitly understood that more material had been prepared similarly.

Microwave reactions were performed in dedicated MW instruments in closed vials.

1H NMR spectra were recorded at 250-600 MHz in d6-DMSO un1 ess stated otherwise.

Reactions performed above the boiling point of the solvent took place in sealed tubes or screw-cap vials.

Some of the final compounds (Examples) were obtained in pure form and the yield provided accordingly while others were obtained as DMSO solutions for which the concentration and volume are specified.

“Dried” refer to drying an organic solution over Na₂SO₄, MgSO₄, or CaCl₂) and filtering off the drying agent.

“Degassed” refers to air-sensitive reactions being flushed with inert atmosphere via evacuation and back-filling or by bubbling inert atmosphere through the mixture for several minutes.

NaH was used as a 60% oil dispersion.

“Filtration” of mixtures refers to the removal/isolation of solid material from mixture by filtration through a paper filter, PFTE septum, or through a pad of celite. Additional solvent was used to wash the solid.

Unless noted otherwise hydrogenations were performed at 1 bar from a balloon.

‘Purification by SCX’ refers to the loading of the material onto a SCX column, washing with MeOH, eluting with NH₃ in MeOH, and concentrating the fractions containing the product.

Catalytic hydrogenations were performed using a catalyst such as 10% Pd/C and a H2 balloon un1 ess stated otherwise. The mixture after the reaction was typically filtered through celite to remove the catalyst.

‘Partitioned (A/B)’ refers to the partitioning of a mixture between solvents A and B.

OL (A/B) refers to the organic layer after partitioning the mixture between solvents A and B.

AL (A/B) refers to the aq. layer after partitioning the mixture between solvents A and B.

Crude reaction mixtures after reductions with iron/zinc power and AcOH/NH₄Cl were typically filtered through celite before work-up of the filtrate.

### HPLC Methods

Several Intermediates and final compounds were purified using the HPLC methods listed below.

| Column | Eluent | Flow rate |
|---|---|---|
| XBridge Prep C18 OBD, | A: 50 mM aq. NH₄HCO₂, B: ACN, 10-100% ACN | 30 mL/min |
| 150 × 19 mm 5 μm | | |
| XTerra ® RP-18 OBD, | A: 0.1% aq. NH₄HCO₂, B: ACN, 10-100% ACN | 30 mL/min |
| 150 × 19 mm 5 μm | | |
| PRINCETONE | A: 0.05% aq. HCO₂H, B: ACN | 16 mL/min |
| ULTIMA C18 250 × 20 | 0 min 20% B, 1 min 20% B, 10 min 70% B, 10.1 | |
| mm, 5 μm | min 99% B, 12 min 99% B, 12.1 min, 20% B, 15 | |
| | min 20% B 12.1/20, 15/20 | |
| PRINCETONE | A: 0.1% aq. HCO₂H, B: ACN | 16 mL/min |
| ULTIMA C18 250 × 20 | 0 min 20% B, 2 min 20% B, 10 min 50% B | |
| mm, 5 μm | | |
| LUNA OMEGA C18 | A: 10 mM aq. NH₄HCO₂, B: ACN | 18 mL/min |
| 250 × 21.2 mm, 5 μm | 0 min 40% B, 1 min 40% B, 10 min 85% B, 10.1 | |
| | min 99% B, 12 min 99% B, 12.1 min 40% B, | |
| | 15 min 40% B | |
| COGENT C18 250 × 21.2 | A: 10 mM aq. NH₄HCO₂, B: ACN | 16 mL/min |
| mm, 5 μm | 0 min 30% B, 2 min 30% B, 10 min 50% B | |
| COGENT C18 250 × 21.2 | A: 10 mM aq. NH₄HCO₂, B: ACN | 20 mL/min |
| mm, 5 μm | 0 min 30% B, 1 min 30% B, 10 min 80% B, 10.1 | |
| | min 99% B, 12 min 99% B, 12.1 min 30% B, 15 | |
| | min 30% B | |
| LUNA C18 150 × 21.2 | A: 10 mM aq. NH₄HCO₂, B: ACN | 18 mL/min |
| mm, 5 μm | 0 min 40% B, 1 min 40% B, 10 min 80% B, 10.1 | |
| | min 99% B, 12 min 99% B, 12.1 min 40% B, 15 | |
| | min 40% B | |
| X-SELECT PHENYL | A: 10 mM aq. NH₄HCO₂, B: ACN | 16 mL/min |
| HEXYL 250 × 19 mm, 5 | 0 min 30% B, 1 min 30% B, 10 min 55% B | |
| μm | | |
| X-SELECT PHENYL | A: 10 mM aq. NH₄HCO₂, B: ACN | 18 mL/min |
| HEXYL 250 × 19 mm, 5 | 0 min 55% B, 1 min 55% B, 10 min 70% B, 15 | |
| μm | min 90% B, 15.1 min 100% B, 19 min 100% B, | |
| | 19.1 min 55% B | |
| AZZOTA 250 × 20 mm, 10 | A: 10 mM aq. NH₄HCO₂, B: ACN | 18 mL/min |
| μm | 0 min 40% B, 1 min 40% B, 10 min 85% B, 10.1 | |
| | min 99% B, 12 min 99% B, 12.1 min 40% B, 15 | |
| | min 40% B | |
| AZZOTA 250 × 20 mm, 5 | A: 10 mM aq. ABC, B: ACN | 17 mL/min |
| μm | 0 min 30% B 1 min 30% B, 10 min 85% B | |
| PRINCETONE | A: 0.1% aq. HCO₂H, B: ACN | 17 mL/min |
| ULTIMA C18 250 × 20 | 0 min 10% B, 1 min 10% B, 10 min 90% B, 10.1 | |
| mm, 10 μm | min 99% B, 13 min 99% B, 13.1 min 10% B, 16 | |
| | min 10% B | |
| XSELECT CSH C18 | A: 0.1% aq. HCO₂H, B: ACN | 17 mL/min |
| 250 × 19 mm 5 μm | 0 min 10% B, 1 min 10% B, 10 min 90% B, 10.1 | |
| | min 99% B, 13 min 99% B, 13.1 min 10% B, 16 | |
| | min 10% B | |
| YMC-PACK-ODS-AQ | A: 0.1% aq. HCO₂H, B: ACN | 15 mL/min |
| C18 250 × 20 mm 5 μm | 0 min 25% B, 2 min 25% B, 10 min 35% B | |
| HICHROME C18 | A: 10 mM aq. ABC, B: ACN | 18 mL/min |
| 250 × 20 mm 5 μm | 0 min 40% B, 2 min 40% B, 10 min 80% B | |
| X SELECTC18 250 × 19 | A: 10 mM aq. ABC, B: ACN | 16 mL/min |
| mm 5 μm | 0 min 40% B, 1 min 40% B, 10 min 80% B, 11 | |
| | min 80% B, 11.1 min 99% B, 15 min 99% B, | |
| | 15.1 min 40% B, 19 min 40% B | |

### LC/MS Method

The compounds of disclosure were characterized by the LC/MS methods listed below.

| Method | Column and eluent | Gradient | Flow rate | |
|---|---|---|---|---|
| 1 | Agilent Poroshell 120 SB-C18 4.6 × 30 | 0 min 1% B, 1.5 min | 3 | mL/min |
| | mm 2.7 μm operated at 60° C. | 100% B, 1.73 min | | |
| | A: 0.1% aq. HCO₂H | 100% B | | |
| | B: 0.1% HCO₂H in ACN | | | |
| 2 | Acquity BEH C18 50 × 2.1 mm 1.7 μm | 0 min 3% B, 0.4 min | 0.6 | mL/min |
| | operated at 35° C. | 3% B, 2.5 min 98% B, | | |
| | A: 0.05% aq. HCO₂H | 3.5 min 98%, 3.6 min | | |
| | B: 0.05% HCO₂H in ACN | 3% B, 4 min 3% B | | |
| 3 | Acquity BEH C18 50 × 2.1 mm 1.7 μm | 0 min 3% B, 2.5 min | 0.6 | mL/min |
| | operated at 35° C. | 3% B, 7.5 min 98% B, | | |
| | A: 0.05% aq. HCO₂H | 9.6 min 3% B, 10 min | | |
| | B: 0.05% HCO₂H in ACN | 3% B | | |
| 4 | Acquity BEH C18 100 × 2.1 mm 1.7 μm | 0 min 3% B, 8.5 min | 0.55 | mL/min |
| | operated at 50° C. | 100% B, 9 min 100% | | |
| | A: 0.05% aq. HCO₂H | B, 9.5 min 3% B, 10 | | |
| | B: 0.05% HCO₂H in ACN | min 3% B | | |
| 5 | Acquity BEH C18 50 × 2.1 mm 1.7 μm | 0 min 3% B, 0.4 min | 0.6 | mL/min |
| | operated at 35° C. | 3% B, 2.5 min 98% B, | | |
| | A: 0.05% aq. TFA | 3.5 min 98% B, 3.6 | | |
| | B: 0.05% TFA in ACN | min 3% B, 4 min 3% B | | |
| 6 | Acquity BEH C18 50 × 2.1 mm 1.7 μm | 0 min 3% B, 2.5 min | 0.6 | mL/min |
| | operated at 35° C. | 3% B, 7.5 min 98% B, | | |
| | A: 0.05% aq. TFA | 9.5 min 98% B, 9.6 | | |
| | B: 0.05% TFA in ACN | min 3% B, 10 min 3% | | |
| | | B | | |
| 7 | Acquity BEH C18 100 × 2.1 mm 1.7 μm | 0 min 3% B, 16 min | 0.45 | mL/min |
| | operated at 50° C. | 100% B, 18 min 100% | | |
| | A: 0.05% aq. TFA | | | |
| | B: 0.05% TFA in ACN | B, 18.5 min 3% B, 20 | | |
| | | min 3% B | | |
| 8 | Xbridge C18 75 × 4.6 mm 3.5 μm | 0 min 5% B, 0.5 min | 1.3 | mL/min |
| | operated at 35° C. | 5% B, 1 min 15% B, 4 | | |
| | A: 10 mM aq. NH₄HCO₃ | min 98% B, 7 min 98% | | |
| | B: ACN | B, 7.5 min 5% B, 8 | | |
| | | min 5% B | | |
| 9 | X Bridge C18 150 × 4.6 mm 3.5 μm | 0 min 5% B, 1 min 5% | 1.0 | mL/min |
| | operated at 35° C. | B, 3 min 15% B, 7 min | | |
| | A: 10 mM aq. NH₄HCO₃ | 55% B, 11 min 98% B, | | |
| | B: ACN | 16 min 98% B, 16.1 | | |
| | | min 5% B, 20 min 5% | | |
| | | B | | |
| 10 | X SELECT C18 150 × 4.6 mm 3.5 μm | 0 min 5% B, 1 min 5% | 1.0 | mL/min |
| | operated at RT | B, 3 min 15% B, 7 min | | |
| | A: 10 mM aq. NH₄HCO₃ | 55% B, 11 min 98% B, | | |
| | B: ACN | 16 min 98% B, 16.1 | | |
| | | min 5% B, 20 min 5% | | |
| | | B | | |
| 11 | Acquity UPLC HSS T3 50 × 2.1 mm 1.8 | 0 min 1% B, 0.5 min | 0.7 | mL/min |
| | μm operated at 30° C. | 6% B, 1 min 6% B, 2.6 | | |
| | A: 10 mM aq. NH₄OAc + 0.1% HCO₂H | min 95% B, 3.8 min | | |
| | B: 0.1% HCO₂H in ACN | 95% B, 3.81 min 1% | | |
| | | B, 4.8 min 1% B | | |
| 12 | Acquity UPLC HSS T3 50 × 2.1 mm 1.8 | 0 min 5% B, 0.9 min | 1.2-1.3 | mL/min |
| | μm operated at 60° C. | 95% B, 1.2 min 95% | | |
| | A: 10 mM aq. NH₄OAc + 0.1% HCO₂H | B, 1.4 min 5% B | | |
| | B: 0.1% HCO₂H in ACN | | | |
| 13 | X Brigde BEH C18 (3 × 100) mm, 2.5 μm | 0 min 5% A, 5 min | 1 | mL/min |
| | operated at 35° C. | 98% A, 9 min 98% A, | | |
| | A: 0.05% TFA in ACN | 9.01 min 5% A, 12 min | | |
| | B: 0.05% aq. TFA | 5% A | | |
| 14 | ACQUITY UPLC BEH C18 (2.1 × 100) | 0 min 10% A, 2.5 min | 0.4 | mL/min |
| | mm, 1.7 μm operated at 60° C. | 10% A, 7.5 min 98% | | |
| | A: 0.05% TFA in ACN | A, 9.5 min 98% A, 9.6 | | |
| | B: 0.05% aq. TFA | min 10% A, 10 min | | |
| | | 10% A | | |
| 15 | YMC-Triart C18 ExRS (75 × 4.6 mm, | 0 min 5% B, 0.8 min | 1.0 | mL/min |
| | 3 μm) operated at 45° C. | 5% B, 2 min 25% B, 5 | | |
| | A: 10 mM aq. NH₄HCO₃ | min 90% B, 7 min 95% | | |
| | B: 100% ACN | B, 8.5 min 95% B, 8.6 | | |
| | | min 5% B, 10 min 5% | | |
| | | B | | |
| 16 | X-BRIDGE C8 (4.6 × 75 mm) 3.5 μm | 0 min 5% B, 3 min | 1.0 | mL/min |
| | operated at 50° C. | 98% B, 5 min 98% B, | | |
| | A: 0.05% aq. HCO₂H | 5.5 min 5% B, 6 min | | |
| | B: 0.05% HCO₂H in ACN | 5% B | | |
| 17 | Xbridge C18 (75 × 4.6 mm, 3 μm) operated | 0 min 5% B, 0.5 min | 1.0 | mL/min |
| | at 50° C. | 5% B, 1 min 15% B, 4 | | |
| | A: 10 mM aq. NH₄HCO₃ | min 98% B, 7 min 98% | | |
| | B: ACN | B, 7.5 min 5% B, 8 | | |
| | | min 5% B | | |
| 18 | Acquity BEH C18 (50 mm × 2.1 mm, | 0 min 5% B, 5 min | 0.6 | mL/min |
| | 1.7 um) operated at 35° C. | 98% B, 9 min 98% B, | | |
| | A: 0.05% aq. HCO₂H | 9.01 min 5% D, 12 min | | |
| | B: 0.05% TFA in ACN | 5% B | | |
| 19 | ACQUITY UPLC BEH C18 (2.1 × 100) | 0 min 10% A, 2.5 min | 0.4 | mL/min |
| | mm, 1.7 μm operated at 45° C. | 10% A, 7.5 min 98% | | |
| | A: 0.05% TFA in ACN | A, 9.5 min 98% A, 9.6 | | |
| | B: 0.05% aq. TFA | min 10% A, 10 min | | |
| | | 10% A | | |
| 20 | ACQUITY UPLC BEH C18 (2.1 × 100) | 0 min 10% A, 2.5 min | 0.4 | mL/min |
| | mm, 1.7 μm operated at 35° C. | 10% A, 7.5 min 98% | | |
| | A: 0.05% TFA in ACN | A, 9.5 min 98% A, 9.6 | | |
| | B: 0.05% aq. TFA | min 10% A, 10 min | | |
| | | 10% A | | |
| 21 | ACQUITY UPLC BEH C18 (2.1 × 100) | 0 min 10% A, 2.5 min | 0.55 | mL/min |
| | mm, 1.7 μm operated at 50° C. | 10% A, 7.5 min 98% | | |
| | A: 0.05% TFA in ACN | A, 9.5 min 98% A, 9.6 | | |
| | B: 0.05% aq. TFA | min 10% A, 10 min | | |
| | | 10% A | | |
| 22 | ACQUITY UPLC BEH C18 (2.1 × 100) | 0 min 10% A, 2.5 min | 0.65 | mL/min |
| | mm, 1.7 μm operated at 35° C. | 10% A, 7.5 min 98% | | |
| | A: 0.05% TFA in ACN | A, 9.5 min 98% A, 9.6 | | |
| | B: 0.05% aq. TFA | min 10% A, 10 min | | |
| | | 10% A | | |
| 23 | X Bridge C18 (50 mm × 4.6 mm, 3.5 μm) | 0 min 5% B, 0.5 min | 0.8 | mL/min |
| | operated at 45° C. | 5% B , 5 min 98% B, | | |
| | A: 10 mM aq. NH₄HCO₃ | 7.5 min 98% B, 7.6 | | |
| | B: ACN | min 5% B, 9 min 5% B | | |
| 24 | YMC-Triart C18 ExRS (50 × 2.1 mm, | 0 min 5% B, 5 min | 0.6 | mL/min |
| | 1.9 μm) operated at 35° C. | 98% B, 9 min 98% B, | | |
| | A: 0.05% aq. TFA | 9.01 min 5% B, 12 min | | |
| | B: 0.05% TFA in ACN | 5% B | | |
| 25 | X Bridge C18 50 × 4.6 mm 3.5 μm | 0 min 2% B, 0.5 min | 0.8 | mL/min |
| | operated at 35° C. | 2% B, 3 min 98% B, 6 | | |
| | A: 10 mM aq. NH₄HCO₃ | min 98% B, 8 min 2% | | |
| | B: ACN | B | | |
| 26 | X Bridge C18 50 × 4.6 mm 3.5 μm | 0 min 5% B, 0.5 min | 1.3 | mL/min |
| | operated at 30° C. | 5% B, 1.0 min 15% B, | | |
| | A: 10 mM aq. NH₄HCO₃ | 4.0 min 98% B, 7.0 | | |
| | B: ACN | min 98% B, 7.5 min | | |
| | | 5% B, 8.0 min 5% B | | |
| 27 | X Bridge C18 50 × 4.6 mm 3.5 μm | 0 min 2% B, 0.5 min | 1.0 | mL/min |
| | operated at 45° C. | 2% B, 2.5 min 98% B, | | |
| | A: 10 mM aq. NH₄HCO₃ | 5 min 98% B, 5.1 min | | |
| | B: ACN | 2% B, 6 min 2% B | | |
| 28 | YMC Triart C18 (75 × 4.6 mm, 3 um) | 0 min 5% B, 0.5 min | 1.3 | mL/min |
| | operated at 45° C. | 5% B, 1 min 15% B, 6 | | |
| | A: 5 mM aq. NH₄HCO₃ | min 55% B, 9 min 95% | | |
| | B: ACN | B, 12 min 95% B, 13 | | |
| | | min 5% B, 14 min 5% | | |
| | | B | | |
| 29 | YMC Triart C18 (75 × 2.1 mm, 1.9 um) | 0 min 3% B, 0.4 min | 0.6 | mL/min |
| | operated at 35° C. | 3% B, 2.5 min 98% B, | | |
| | A: 0.05% aq. TFA | 3.5 min 98% B, 3.6 | | |
| | B: 0.05% TFA in ACN | min 3% B, 4 min 3% B | | |
| 30 | YMC Triart C18 (50 × 2.1 mm, 1.9 um) | 0 min 3% A, 2.5 min | 0.6 | mL/min |
| | operated at 60° C. | 3% A, 7.5 min 98% A, | | |
| | A: 0.05% aq. TFA | 9.5 min 98% A, 9.6 | | |
| | B: 0.05% TFA in ACN | min 3% A, 10 min 3% | | |
| | | A | | |
| 31 | YMC Triart C18 (50 × 2.1 mm, 1.9 um) | 0 min 3% A, 0.4 min | 0.6 | mL/min |
| | operated at 60° C. | 3% A, 2.5 min 98% A, | | |
| | A: 0.05% aq. TFA | 3.5 min 98% A, 3.6 | | |
| | B: 0.05% TFA in ACN | min 3% A, 4 min 3% A | | |
| 32 | YMC Triart C18 (50 × 4.6 mm, 1.9 um) | 0 min 5% A, 0.5 min | 1.0 | mL/min |
| | operated at 60° C. | 5% A, 3 min 95% A, 6 | | |
| | A: 0.05% TFA in ACN | min 95% A, 6.1 min | | |
| | B: 0.05% aq. TFA | 5% A, 8 min 5% A | | |
| 33 | X Bridge C18 75 × 4.6 mm 3.5 μm | 0 min 5% B, 0.5 min | 1.0 | mL/min |
| | operated at 45° C. | 5% B, 1 min 15% B, 6 | | |
| | A: 5 mM aq. NH₄HCO₃ | min 55% B, 9 min 95% | | |
| | B: ACN | B, 12 min 95% B, 13 | | |
| | | min 5% B, 14 min 5% | | |
| | | B | | |
| 34 | X Bridge C18 75 × 4.6 mm 3.5 μm | 0 min 5% B, 0.5 min 5 | 1.3 | mL/min |
| | operated at 35° C. | % B, 1.0 min 15% B, | | |
| | A: 10 mM aq. NH₄HCO₃ | 4.0 min 98% B, 7.0 | | |
| | B: ACN | min 98% B, 7.5 min | | |
| | | 5% B, 8.0 min 5% B | | |
| 35 | X Brigde BEH C18 (3 × 100) mm, 2.5 μm | 0 min 3% A, 4 min | 1 | mL/min |
| | operated at 35° C. | 98% A, 5 min 98% A, | | |
| | A: 0.05% TFA in ACN | 5.01 min 3% A, 6 min | | |
| | B: 0.05% aq. TFA | 3% A | | |
| 36 | X Bridge C18 (100 mm × 3 mm, 2.5 μm) | 0 min 3% A, 4 min | 1.0 | mL/min |
| | operated at 50° C. | 98% A, 5 min 98% A, | | |
| | A: 0.05% aq. TFA | 5.01 min 3% A, 6 min | | |
| | B: 0.05% TFA in ACN | 3% A | | |
| 37 | X Bridge C18 (75 mm × 4.6 mm, 3.5 μm) | 0 min 5% B, 0.8 min | 1.0 | mL/min |
| | operated at 45° C. | 5% B, 2 min 25% B, 5 | | |
| | A: 10 mM aq. NH₄HCO₃ | min 90% B, 7 min 95% | | |
| | B: 100% ACN | B, 8.6 min 5% B, 10 | | |
| | | min 5% B | | |
| 38 | Acquity BEH C18 100 × 2.1 mm 1.7 μm | 0 min 3% A, 1 min | 0.55 | mL/min |
| | operated at 60° C. | 10% A, 8.5 min 100% | | |
| | A: 0.05% TFA in ACN | A, 9 min 100% A, 9.5 | | |
| | B: 0.05% aq. TFA | min 3% A, 10 min 3% | | |
| | | A | | |
| 39 | Acquity BEH C18 100 × 2.1 mm 1.7 μm | 0 min 3% A, 8 min | 0.45 | mL/min |
| | operated at 60° C. | 100% A, 9 min 100% | | |
| | A: 0.05% aq. TFA | A, 9.5 min 3% A, 10 | | |
| | B: 0.05% TFA in ACN | min 3% A | | |
| 40 | Acquity BEH C18 100 × 2.1 mm 1.7 μm | 0 min 10% B, 2.5 min | 0.4 | mL/min |
| | operated at 45° C. | 10% B, 7.5 min 98% | | |
| | A: 0.05% aq. TFA | B, 9.5 min 98% B, 9.6 | | |
| | B: 0.05% TFA in ACN | min 10% B, 10 min | | |
| | | 10% B | | |
| 41 | Agilent Poroshell 120 SB-C18 | 0 min 1% B, 1.5 min | 3 | mL/min |
| | 4.6 × 30 mm 2.7 μm operated at 45° C. | 100% B, 2.2 min 100% | | |
| | A: 0.1% aq HCO₂H | B | | |
| | B 0.1% HCO₂H in ACN | | | |
| 42 | Waters HSS T3 1.8 μm, 1.0 × 50 mm | 0 min 10% B, 0.1 min | 0.475 | mL/min |

| | |
|---|---|
| column operated at 50° C. | 10% B, 0.6 min 95% |
| A: 0.01% aq HCO₂H | B, 1.5 min 95% B, 1.51 |
| B 0.01% HCO₂H in AC | min 10% B |

### Abbreviations

ABPR=automated back pressure regulator
ACN=acetonitrile
AcOH=acetic acid
AdBrettPhos Pd G3=[2-(di-1-adamantyl-phosphino)-2′,4′,6′-tri-iso-propyl-3,6-dimethoxybiphenyl] [2-(2′-amino-1,1′-biphenyl)]palladium(II) methanesulfonate
aq=aqueous
Boc=tert-butoxycarbonyl
Boc₂O=di-tert-butyl dicarbonate
BOP=(benzotriazol-1-yloxy) tris(dimethylamino)-phosphonium hexafluorophosphate
B₂Pin₂=4,4,4′, 4′, 5,5,5′, 5′-octamethyl-2,2′-bi (1,3,2-dioxaborolane) Brettphos Pd G3=[(2-Di-cyclo-hexylphosphino-3,6-dimethoxy-2′,4′,6′-tri-iso-propyl-1,1′-biphenyl)-2-(2′-amino-1,1′-biphenyl)]palladium(II) methanesulfonate methanesulfonate
BrettPhos Pd G4=[2′-(methylamino)-[1,1′-biphenyl]-2-yl] palladio methanesulfonate di-cyclo-hexyl [3,6-dimethoxy-2′,4′,6′-tris (propan-2-yl)-[1,1′-biphenyl]-2-yl] phosphane
brine=saturated aqueous sodium chloride
cataCXium Pd G4=[di (adamantan-1-yl) (butyl) phosphine] (methanesulfonato-KO) [2′-(methylamino)-2-biphenylyl] palladium
CBz=benzyloxycarbonyl
CBz-Cl=benzyl chloroformate
CDI=1,l′-carbonyldiimidazole
DAST=diethylaminosulfur trifluoride
dba=dibenzylideneacetone
DCC=di-cyclo-hexylcarbodiimide
DCE=1,2-dichloroethane
DCM=dichloromethane
DEA=diethylamine
DEAD=diethyl azodicarboxylate
Deoxo-Fluor=bis(2-methoxyethyl)aminosulfur trifluoride
DIAD=di-iso-propyl azodicarboxylate
DIPEA=di-iso-propyl ethyl amine
DMAP=4-(dimethylamino)pyridine
DME=1,2-dimethoxyethane
DMF=dimethyl formamide
DMDCH=trans-N,N′-dimethyl-cyclo-hexane-1,2-diamine
DMEDA=N1,N2-dimethylethane-1,2-diamine
DMP=Dess-Martin periodinane
DMS=dimethyl sulfide
DMSO=dimethyl sulfoxide
DPPF=1,1′-bis(diphenylphosphino)ferrocene
EDC=N-(3-dimethylaminopropyl)-N′-ethylcarbodi-imide
EtOAc=ethyl acetate
EtOH=ethanol
FC=flash chromatography on silica gel un1 ess stated otherwise from the eluent described in the brackets
h=hour(s)
HATU=(1-[bis(dimethylamino)methylene]-1H-1,2,3-triazolo[4,5-b]pyridinium 3-oxide hexafluorophosphate
HBTU=N,N,N′,N′-tetramethyl-O-(1H-benzotriazol-1-yl) uronium hexafluorophosphate
HFIP=1,1,1,3,3,3-hexafluoro-2-propanol
HOBt=hydroxybenzotriazole
HPLC=high performance liquid chromatography
Int./Ints.=intermediate/intermediates
IPA=iso-propyl alcohol
KOAc=potassium acetate
KOtBu=potassium tert-butoxide
LCMS=liquid chromatography-mass spectrometry
LDA=lithium di-iso-propylamide
LiHMDS=lithium bis(trimethylsilyl) amide
mCPBA=m-chloro-perbenzoic acid
MeOH=methanol
2MeTHF=2-methyl-tetrahydrofuran
MHz=megahertz
MS=molecular sieves
MsCl=methanesulfonyl chloride (mesyl chloride)
MTBE=methyl tert-butyl ether
MW=microwave
NaOtBu=sodium tert-butoxide
NBS=N-bromosuccinimide
NMP=N-methyl-2-pyrrolidon
NMR=nuclear magnetic resonance
ON=overnight
Pd₂dba₃=tris(dibenzylideneacetone)dipalladium(0)
PdDPPFCl₂-DCM=[1,1′-bis(diphenylphosphino)-ferrocene]dichloropalladium(II) DCM complex
Pd G3 SPhos=(2-Di-cyclo-hexylphosphino-2′,6′-dimethoxybiphenyl) [2-(2′-amino-1,1′-biphenyl)]palladium(II) methanesulfonate
Pd G3 tert-butyl-Xphos=methanesulfonato (di-tert-butyl)phenylphosphino (2′-amino-1,1′-biphenyl-2-yl) palladium(II)
ppm=parts per million
PyBOP=(benzotriazol-1-yloxy)tripyrrolidino-phosphonium hexafluorophosphate RT=room temperature
RU=response units
RuPhos=2-di-cyclo-hexylphosphino-2′,6′-di-iso-propoxybiphenyl
Ruphos Pd-G2=Chloro (2-dicyclohexylphosphino-2′,6′-diisopropoxy-1, 1′-biphenyl) [2-(2′-amino-1,1′-biphenyl)]palladium(II)
sat. =saturated
SCX=strong cation exchange
SEM=2-(trimethylsilyl) ethoxymethyl
SFC=supercritical fluid chromatography
SPR=Surface plasmon resonance
STAB=sodium triacetoxy borohydride
TBAF=tetra n-butyl ammonium fluoride
TBAI=tetra n-butyl ammonium iodide
TFA=trifluoro acetic acid
Tf2O=trifluoromethanesulfonic anhydride
THF=tetrahydrofuran
TLC=thin layer chromatography
TMEDA=tetramethylethylenediamine
Ts=tosyl
TsOH=p-toluenesulfonic acid
T3P=propanephosphonic acid anhydride
VCD=Vibrational circular dichroism
XantPhos=4,5-bis(diphenylphosphino)-9,9-dimethylxanthene
XPhos=2-di-cyclo-hexylphosphino-2′,4′,6′-tri-iso-propylbiphenyl

### Synthesis Methods

The compounds of the present disclosure can be prepared in a number of ways well known to those skilled in the art of synthesis. The compounds of the disclosure could for example be prepared using the reactions and techniques outlined below together with methods known in the art of synthetic organic chemistry, or variations thereof as appreciated by those skilled in the art. Preferred methods include, but are not limited to, those described below. The reactions are carried out in solvents appropriate to the reagents and materials employed and suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognized by one skilled in the art. Not all compounds falling into a given class may be compatible with some of the reaction conditions required in some of the methods described. Such restrictions to the substituents which are compatible with the reaction conditions will be readily apparent to one skilled in the art and alternative methods can be used.

The compounds of the present disclosure or any intermediate could be purified, if required, using standard methods well known to a synthetic organist chemist, e.g, methods described in “Purification of Laboratory Chemicals”, 6th ed. 2009, W. Amarego and C. Chai, Butterworth-Heinemann.

Starting materials are either known or commercially available compounds, or may be prepared by routine synthetic methods well known to a person skilled in the art.

Unless otherwise noted, reagents and solvents were used as received from commercial suppliers. The organic solvents used were usually anhydrous. The solvent ratios indicated refer to vol: vol un1 ess otherwise noted. Thin layer chromatography was performed using Merck 6OF254 silica-gel TLC plates. Visualization of TLC plates was performed using UV light (254 nm) or by an appropriate staining technique.

The compounds of the disclosure can be prepared according to the key bond formations outlined below.

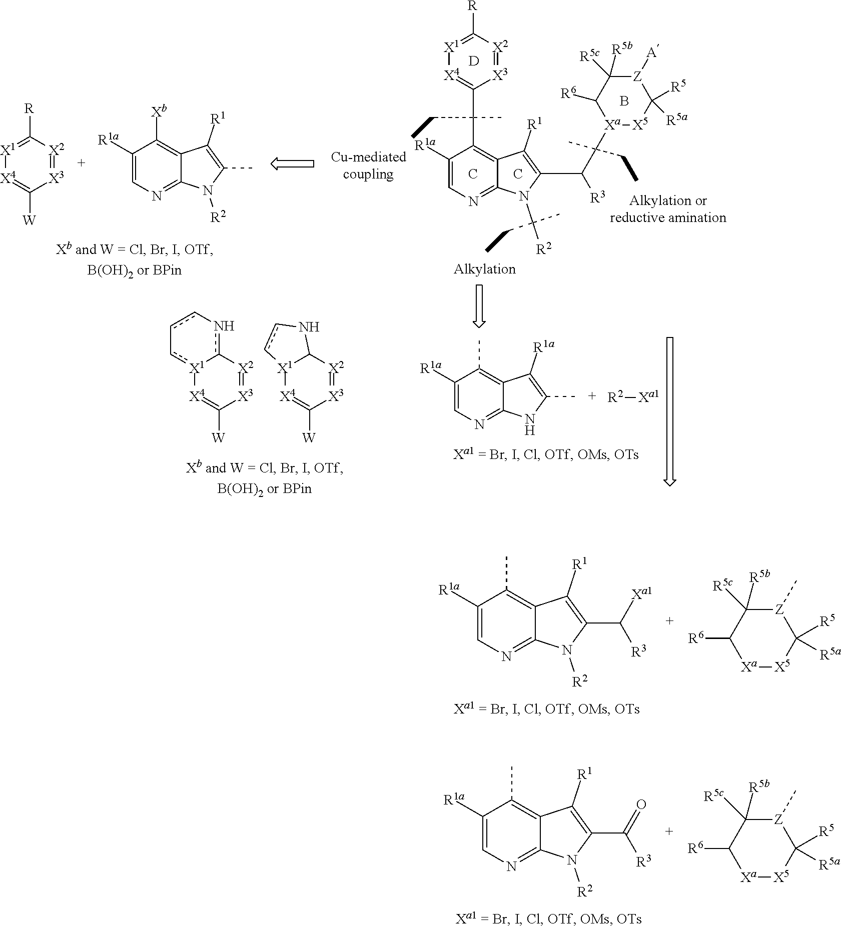

Linking-A-L-LBM can be achieved according to conditions as described below.

Linking the A′ and B rings can be achieved in palladium-mediated couplings reactions wherein either an amine (Z=N) is reacted with an aryl (pseudo) halide based on ring A′ or by coupling ring B in which Z is a carbon atom linked to a boronic acid or boronate to aryl (pseudo) halide based on ring A′. Such reactions can be performed as described for the syntheses of Ints. 1M51 and 1S3.

The bond connecting the B ring to the azaindole core (C—C) can be formed either by alkylation of the B ring amine or lactam with an alkyl (pseudo) halide liked to the azaindole core (C—C) or by reductive amination of the B ring amine to the ketone/aldehyde liked to the azaindole core (C—C). Such reactions can be performed as described for the synthesis of Ints. 1AB2/1AB3, Example 1n7 from Int. 104, Example 1q2 from Int. 1Q1.

R² can be attached to the parent azaindole core (C—C) by alkylation with a suitable alkylation agent. Such reactions can be performed as described for the syntheses of Int. 1K3. Compounds wherein R¹ is not hydrogen can be prepared from precursors like Int. 1H2 or 1Y³.

Attaching ring D to the azaindole core (C—C) can be done either by copper-mediated coupling of a boronic acid or boronate linked to the azaindole (C—C) core and the heteroaromatic ring D bearing a (pseudo) halogen or by the inverted set of Suzuki reaction partners. Such reactions can be performed as described for the synthesis of Example 1af50 from Int. 1AF75 or Example 1af52 from 1AF76′. Compounds in which ring D is a part of a bicyclic system can be prepared similarly as described for the synthesis of Example 2f4 from Int. 3E261 via Int. 3E8 or Example 2c7 from Int. 2C38.

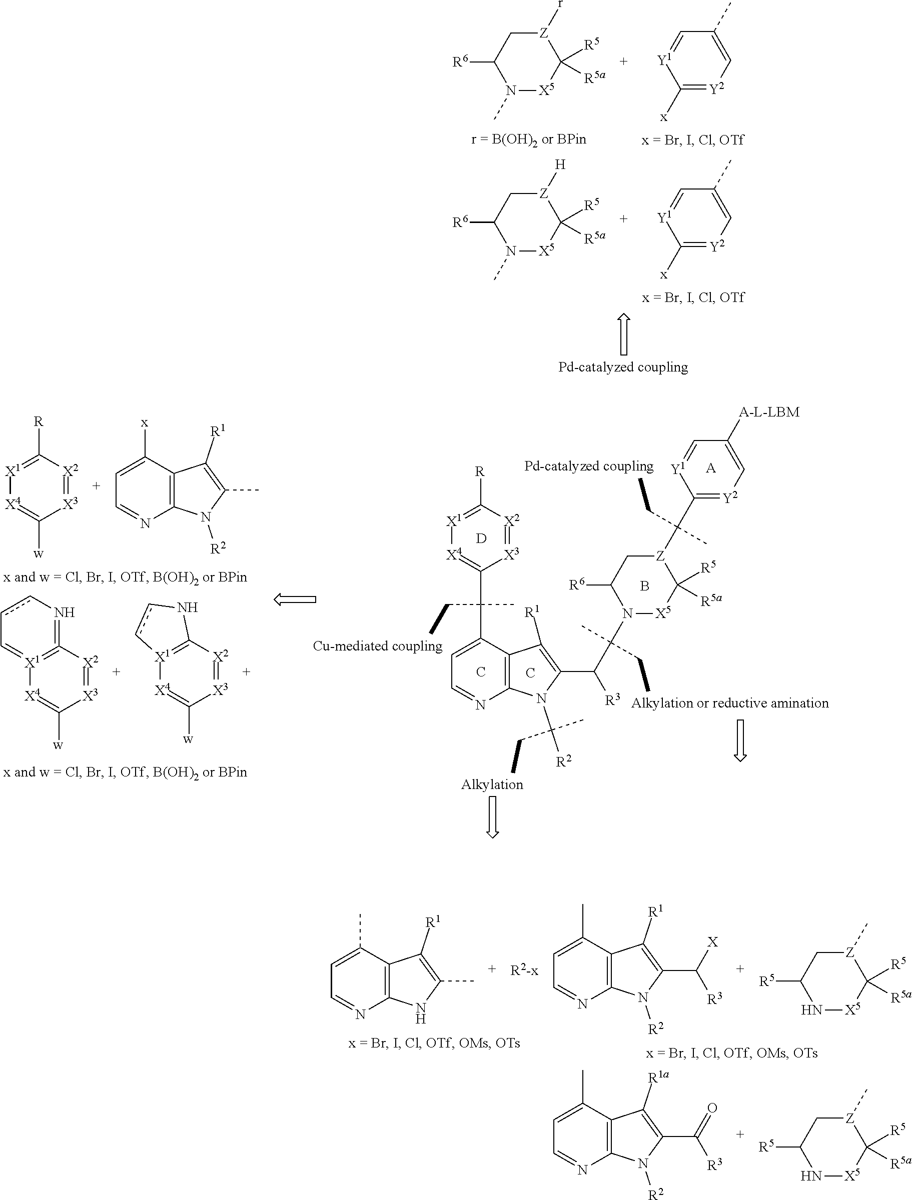

Linking the A and B rings can be achieved in palladium-mediated couplings reactions wherein either an amine (Z=N) is reacted with an aryl (pseudo) halide based on ring A or by coupling ring B in which Z is a carbon atom linked to a boronic acid or boronate to aryl (pseudo) halide based on ring A. Such reactions can be performed as described for the syntheses of Ints. 1M51 and 1S3.

The bond connecting the Bring to the azaindole core (C—C) can be formed either by alkylation of the B ring amine or lactam with an alkyl (pseudo) halide liked to the azaindole core (C—C) or by reductive amination of the B ring amine to the ketone/aldehyde liked to the azaindole core (C—C). Such reactions can be performed as described for the synthesis of Ints. 1AB2/1AB3, Example 1n7 from Int. 104, Example 1q2 from Int. 1Q1.

R² can be attached to the parent azaindole core (C—C) by alkylation with a suitable alkylation agent. Such reactions can be performed as described for the syntheses of Int. 1K3. Compounds wherein R¹ is not hydrogen can be prepared from precursors like Int. 1H2 or 1Y³.

Attaching ring D to the azaindole core (C—C) can be done either by copper-mediated coupling of a boronic acid or boronate linked to the azaindole (C—C) core and the heteroaromatic ring D bearing a (pseudo) halogen or by the inverted set of Suzuki reaction partners. Such reactions can be performed as described for the synthesis of Example 1af50 from Int. 1AF75 or Example 1af52 from 1AF76′. Compounds in which ring D is a part of a bicyclic system can be prepared similarly as described for the synthesis of Example 2f4 from Int. 3E261 via Int. 3E8 or Example 2c7 from Int. 2C38.

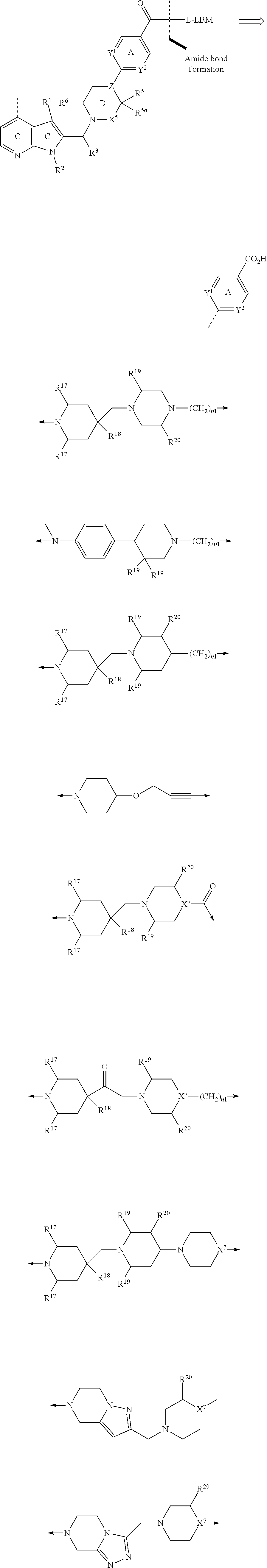

Compounds in which A=CO and the -L-fragment can be introduced via amide bond formation. The prerequisite acids can for example be prepared as described herein for Int. 3E83. The specification exemplifies L-LBM fragments such as Int. 3E146 and their coupling to give the compounds of the disclosure such as described for the reaction of Ints. 3E83/2Q16 to give Example 2q6.

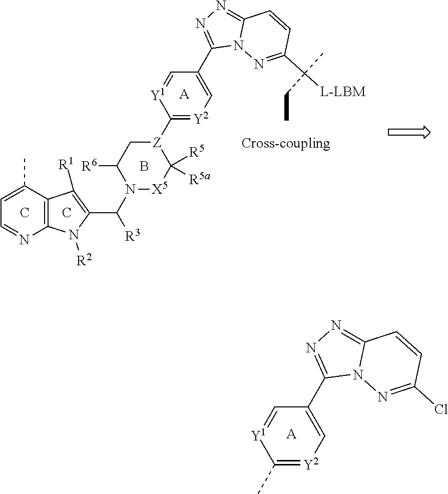

Compounds in which A is a [1,2,4]triazolo[4,3-b]pyridazine unit can be prepared from intermediates like Int. 3E297 by cross-coupling to install the L-LBM fragment as described herein for the synthesis of Example 2r1 via Ints. 3E297/2C15.

Compounds in which the L-LBM motif contains a triple bond can be prepared via alkylation reactions from precursors like Ints. 3A1/2T12 to give Example 2t5. The aromatics rings in the L linker can be functionalized with CN, F, or OMe substituents such as in Examples 2i3, 2i1, and 2i7 which can be prepared as described herein from Ints. 2I17, 2I1, and 2I29. Compounds in which the L contains a six-membered heteroaromatic ring like pyrimidine or pyridine are exemplified herein in Examples 2k1 and 2n3 which can be prepared as described herein from Ints. 2K2 and 2N14.

The LBM piperidine-2,6-dione unit can be introduced by alkylation with 3-bromopiperidine-2,6-dione under the conditions outlined herein for the synthesis of Int. 2F16 en route to Example 2b1.

### Example 1. Preparation of Intermediates

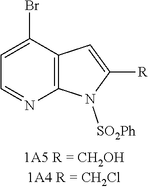

4-Bromo-1-(phenyl-sulfonyl)-1H-pyrrolo[2,3-b]pyridine-2-carbaldehyde (0.27 g) and NaBH₄ (33 mg) were stirred 0.5 h in THF/MeOH (9:1, 5 mL) at 0° C. The OL (water/EtOAc) was washed with brine, dried, and concentrated to give Int. 1A5(4-bromo-1-(phenylsulfonyl)-1H-pyrrolo[2,3-b]pyridin-2-yl) methanol (281 mg). Int. 1A5 (0.24 g) and MsCl (0.052 mL) was stirred 0.5 h in DCM/Et₃N (29:1, 5.2 mL). MsCl (0.014 mL) was added and stirring continued 0.5 h. The OL (water/DCM) was washed with brine, dried, concentrated, and purified by FC (heptane/EtOAc 1:0 to 1:1) to give Int. 1A4 4-bromo-2-(chloromethyl)-1-(phenylsulfonyl)-1H-pyrrolo[2,3-b]pyridine (0.15 g).

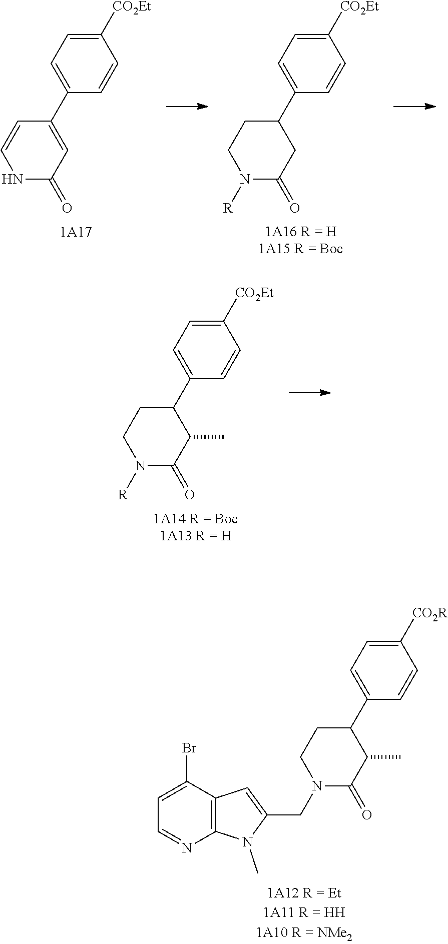

4-Bromopyridin-2 (1H)-one (1.0 g), ethyl 4-(4,4,5,5-tetramethyl-1,3,2-dioxaborolan-2-yl)benzoate (3.17 g), PdDPPFCl₂-DCM (0.42 g), and K₃PO₄ (3.66 g) were degassed in dioxane/water (10:1, 11 mL) and stirred 2 h at 110° C. under MW conditions. The mixture was filtered, concentrated, and purified by FC (DCM/MeOH 95:5) to give Int. 1A17 ethyl 4-(2-oxo-1H-pyridin-4-yl)benzoate (0.50 g). Int. 1A17 (3.5 g) was hydrogenated 48 h using 10% Pd/C (1.5 g) in EtOH/AcOH (4:1, 50 mL), filtered, concentrated, and purified by FC (hexane/EtOAc 7:3) to give Int. 1A16 ethyl 4-(2-oxopiperidin-4-yl)-benzoate (2.5 g). Int. 1A16 (0.50 g), Boc₂O (0.61 g), and DMAP (23 mg) were stirred ON in DCM/Et₃N (25:1, 21 mL) at 0° C. to RT ON. The OL (EtOAc/water) was washed with brine, dried, concentrated, and purified by FC (hexane/EtOAc 7:3) to give Int. 1A15 tert-butyl 4-(4-ethoxy-carbonylphenyl)-2-oxo-piperidine-1-carboxylate (0.40 g). Int. 1A15 (0.30 g) was stirred 0.5 h in 0.1M LiHMDS in THF (11 mL) at −78° C. CH₃I (0.16 mL) was added and stirring continued 2 h at −25 to −30° C. The OL (sat. aq. NH₄Cl/EtOAc) was washed with water and brine, dried, concentrated, and purified by FC (hexane/EtOAc 9:1) to give Int. 1A14 tert-butyl trans-4-(4-ethoxycarbonylphenyl)-3-methyl-2-oxo-piperidine-1-carboxylate (0.18 g). Int. 1A14 (0.60 g) was stirred ON in DCM/TFA (32:1, 20.6 mL) at 0° C. to RT and concentrated. The OL (10% MeOH in DCM/sat. aq. NaHCO₃) was dried, and concentrated to give Int. 1A13 ethyl 4-[trans-3-methyl-2-oxo-4-piperidyl]benzoate (0.38 g). Int. 1A13 (0.20 g) and NaH (28 mg) were stirred 0.5 h in THF (10 mL) at 0° C. to RT. Int. 1J1 (0.22 g) and TBAI (25 mg) were added and stirring continued ON. The OL (aq. citric acid/EtOAc) was dried and concentrated to give Ints. 1A12/1A11 ethyl 4-[trans-1-[(4-bromo-1-methyl-pyrrolo[2,3-b]pyridin-2-yl)methyl]-3-methyl-2-oxo-4-piperidyl]benzoate and 4-[trans-1-[(4-bromo-1-methyl-pyrrolo[2,3-b]pyridin-2-yl)methyl]-3-methyl-2-oxo-4-piperidyl]benzoic acid (0.30 g). 20 mg of this mixture, HNMe₂ (0.11 g, HCl salt), and HATU (0.25 g) were stirred ON in DMF/DIPEA (28.6:1, 10.4 mL). The mixture was combined with another batch prepared similarly on 30 mg scale, concentrated, and purified by FC (hexane/EtOAc 1:4) to give Int. 1A10 N,N-Dimethyl-4-[trans-1-[(4-bromo-1-methyl-pyrrolo[2,3-b]pyridin-2-yl)methyl]-3-methyl-2-oxo-4-piperidyl] benzamide (0.20 g).

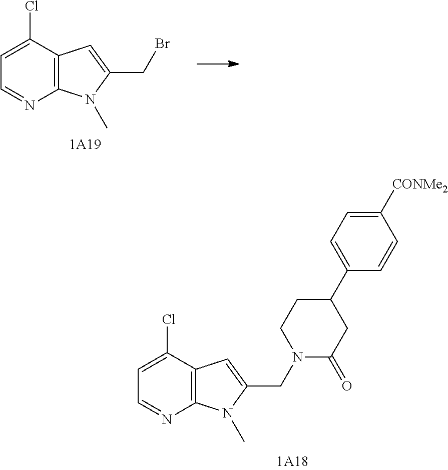

Int. 1A18 4-[1-[(4-Chloro-1-methyl-pyrrolo[2,3-b]pyridin-2-yl)methyl]-2-oxo-4-piperidyl]-N,N-dimethyl-benzamide was prepared similarly to Int. 1A10 from Int. 1A16 and 4-chloro-2-(chloromethyl)-1-methyl-1H-pyrrolo[2,3-b]pyridine (prepared similarly to Int. 1J1).

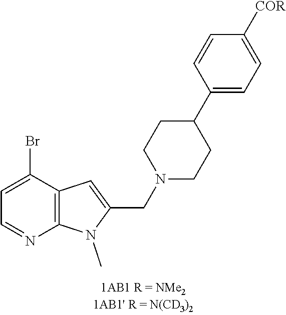

Int. 1J1 (8 g) and N,N-dimethyl-4-(4-piperidyl)benzamide (9.9 g, HCl salt) were stirred ON in DMF/DIPEA (0.7:1, 47 mL) at 0° C. to RT. The OL (water/EtOAc) was washed with brine, dried, concentrated, and purified by FC (hexane/EtOAc 1:3) to give Int. 1AB1 4-(1-((4-Bromo-1-methyl-1H-pyrrolo[2,3-b]pyridin-2-yl)methyl) piperidin-4-yl)-N,N-dimethylbenzamide (9 g). Int. 1AB1′ 4-(1-((4-bromo-1-methyl-1H-pyrrolo[2,3-b]pyridin-2-yl)methyl) piperidin-4-yl)-N,N-bis(methyl-d₃) benzamide was prepared similarly from HN(CD₃)₂.

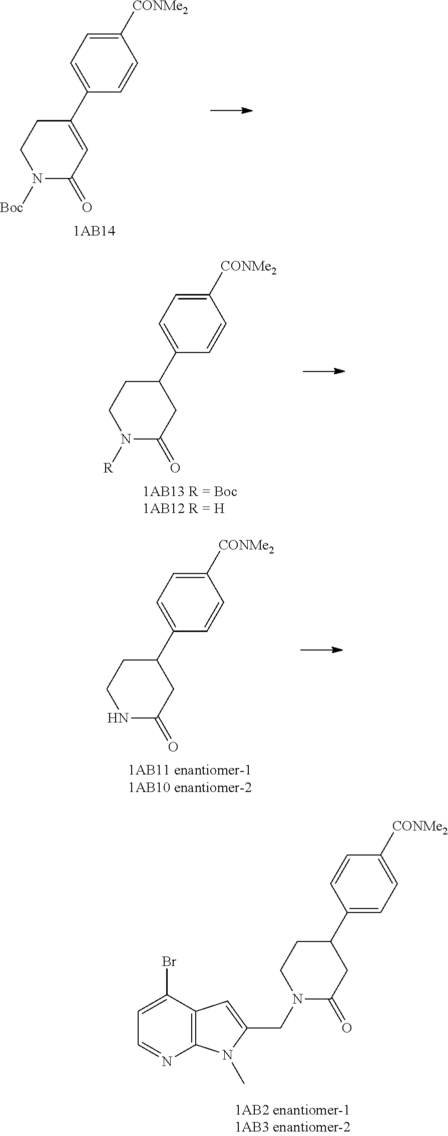

Na₂CO₃ (9.2 g), 1,1,1-trifluoro-N-phenyl-N-((trifluoromethyl) sulfonyl) methane-sulfonamide (10 g), and (4-(dimethylcarbamoyl)phenyl) boronic acid (5.5 g) were degassed in toluene/water (4:1, 250 mL) and stirred 3 h at 100° C. The mixture was filtered, concentrated, and purified by FC (hexane/EtOAc 2:3) to give Int. 1AB14 tert-butyl 4-[4-(dimethylcarbamoyl)phenyl]-6-oxo-2,3-dihydropyridine-1-carboxylate (5.1 g). Int. 1AB14 (4.4 g) was hydrogenated ON using 10% Pd/C (0.45 g) in MeOH (100 mL), filtered, and concentrated to give Int. 1AB13 tert-butyl 4-(4-(dimethylcarbamoyl)-phenyl)-2-oxopiperidine-1-carboxylate (3.9 g). Int. 1AB13 (8.1 g) was stirred ON in DCM/4M HCl in dioxane (4.2:1, 124 mL) and concentrated. The OL (sat. aq. NaHCO₃/10% MeOH in DCM) was dried and concentrated to give Int. 1AB12 N,N-dimethyl-4-(2-oxo-piperidin-4-yl)-benzamide (2.9 g). This material was resolved by SFC using a Chiralpak IG 250×25 5 μm column operated at 30° C. and an eluent of 50% CO₂ and 50% MeOH (100 g/min) and a back pressure of 100 bar to give Int. 1AB11 (0.93 g, first peak) and Int. 1AB12 (0.91 g, second peak). The absolute configurations of these compounds were not determined. Int. 1AB12 (95 mg) and NaH (20 mg) were stirred 0.25 h in DMF (1 mL). Int. 1J1 (0.10 g) was added and stirring continued 0.5 h. The OL (DCM/water) was washed with water and brine, dried, concentrated, and triturated in ACN to give Int. 1AB3 (S)-4-(1-((4-Bromo-1-methyl-1H-pyrrolo[2,3-b]pyridin-2-yl)methyl)-2-oxopiperidin-4-yl)-N,N-dimethylbenzamide or (R)-4-(1-((4-bromo-1-methyl-1H-pyrrolo[2,3-b]pyridin-2-yl)methyl)-2-oxopiperidin-4-yl)-N,N-dimethyl-benzamide (31 mg). Int. 1AB2
(S)-4-(1-((4-Bromo-1-methyl-1H-pyrrolo[2,3-b]pyridin-2-yl)methyl)-2-oxopiperidin-4-yl)-N,N-dimethylbenzamide or (R)-4-(1-((4-bromo-1-methyl-1H-pyrrolo[2,3-b]pyridin-2-yl)methyl)-2-oxopiperidin-4-yl)-N,N-dimethyl-benzamide (35 mg) was prepared similarly from Ints. 1AB11/1J1 (95 mg/0.10 g). The absolute configurations of these compounds were not determined.

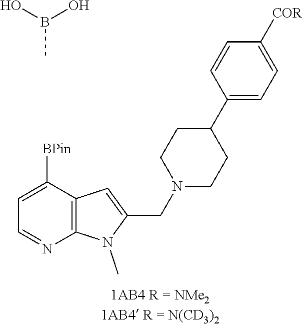

Int. 1AB1 (0.15 g), B₂Pin₂ (0.13 g), KOAc (81 mg), and PdDPPFCl₂-DCM (27 mg) were degassed in dioxane (3 mL) and stirred at 90° C., filtered and concentrated to give Ints. 1AB4/1AB4′ N,N-Dimethyl-4-[1-[1-methyl-4-(4,4,5,5-tetramethyl-1,3,2-dioxaborolan-2-yl) pyrrolo[2,3-b]pyridin-2-yl] methyl]-4-piperidyl] benzamide and [2-[4-[4-(dimethylcarbamoyl)phenyl]-1-piperidyl]methyl]-1-methyl-pyrrolo[2,3-b]pyridin-4-yl]boronic acid and N,N-bis(methyl-d₃)-4-(1-((1-methyl-4-(4,4,5,5-tetramethyl-1,3,2-dioxaborolan-2-yl)-1H-pyrrolo[2,3-b]pyridin-2-yl)methyl) piperidin-4-yl)benzamide and (2-((4-(4-(bis-(methyl-d₃)carbamoyl)phenyl) piperidin-1-yl)methyl)-1-methyl-1H-pyrrolo[2,3-b]pyridin-4-yl) boronic acid.

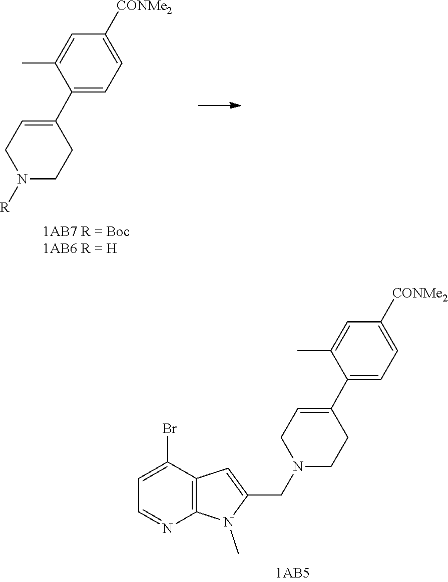

4-Bromo-N,N,3-trimethylbenzamide (9.0 g), tert-butyl 4-(4,4,5,5-tetramethyl-1,3,2-dioxaborolan-2-yl)-3,6-dihydro-2H-pyridine-1-carboxylate (15.2 g), PdDPPFCl₂-DCM (0.67 g), and Na₂CO₃ (9.62 g) were degassed in dioxane (180 mL) and stirred ON at 90° C. The mixture was filtered, concentrated, and purified by FC (hexane/EtOAc 2:3) to give Int. 1AB7 tert-butyl 4-[4-(dimethylcarbamoyl)-2-methyl-phenyl]-3,6-dihydro-2H-pyridine-1-carboxylate (11.0 g). Int. 1AB7 (0.6 g) was stirred ON in DCM/TFA (30:1, 15.5 mL), concentrated, and triturated in Et₂O to give Int. 1AB6 N,N,3-trimethyl-4-(1,2,3,6-tetrahydropyridin-4-yl)benzamide (TFA salt). Ints. 1AB6/1AB8 (0.23 g, TFA salt/0.1 g) were stirred ON in DCE/DIPEA (11.4:1, 5.4 mL) at 0° C. to RT. STAB (26 mg) was added and stirring continued 2 h at 0° C. to RT. The OL (water/DCM) was dried, concentrated, and purified by FC (MeOH/DCM 1:9) to give Int. 1AB5 4-(1-((4-Bromo-1-methyl-1H-pyrrolo[2,3-b]pyridin-2-yl)methyl)-1,2,3,6-tetrahydropyridin-4-yl)-N,N,3-trimethylbenzamide (0.10 g).

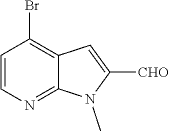

4-Bromo-1-methyl-1H-pyrrolo[2,3-b]pyridine (5.0 g) was stirred 2 h in 1.1M LDA in THF (33 mL) at −78° C. DMF (3.46 g in THF (20 mL)) was added and stirring continued ON at −78° C. to RT ON. The OL (sat. aq. NH₄Cl/EtOAc) was washed with water, brine, dried, concentrated, and triturated in EtOAc/pentane to give Int. 1AB8 4-Bromo-1-methyl-1H-pyrrolo[2,3-b]pyridine-2-carbaldehyde (3.1 g).

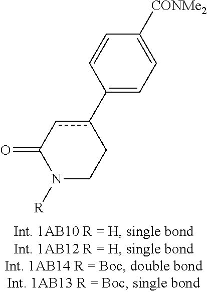

PdDPPFCl₂-DCM (1.1 g), tert-Butyl 6-oxo-4-(((trifluoromethyl) sulfonyl)oxy)-3,6-dihydropyridine-1(2H)-carboxylate (10.2 g), (4-(dimethylcarbamoyl)phenyl) boronic acid (5.7 g), and Na₂CO₃ (9.4 g) were stirred 3 h in toluene/water (150/40 mL) at 100° C. The mixture was filtered. The filtrate was concentrated and purified by FC (EtOAc/Hexane 3:2) to give Int. 1AB14 tert-butyl 4-(4-(dimethyl-carbamoyl)-phenyl)-6-oxo-3,6-dihydropyridine-1(2H)-carboxylate (5.2 g). Int. 1AB14 (5 g) and 10% Pd/C (2.5 g) were hydrogenated 48 h under a hydrogen pressure of 60 psi in EtOAc (100 mL). The mixture was filtered and concentration to give Int. 1AB13 tert-butyl 4-(4-(dimethylcarbamoyl)phenyl)-2-oxopiperidine-1-carboxylate (4.0 g). Int. 1AB13 (0.1 g) was stirred 12 h in DCM/4M HCl in dioxane (5/0.4 mL). The residue after concentration was triturated in Et₂O to give Int. 1AB10/1AB12 N,N-dimethyl-4-(2-oxopiperidin-4-yl)benzamide (78 mg).

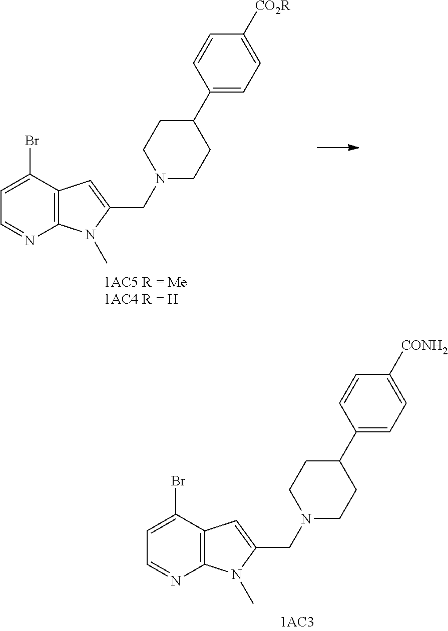

Int. 1AB8 (6.0 g) and methyl 4-(1-piperidin-4-yl)benzoate (7.1 g, HCl salt) were stirred 4 h in DCE/DIPEA (7.7:1, 113 mL). STAB (11.0 g) was added and stirring continued ON at 0° C. to RT. The OL (water/EtOAc) was washed with brine, dried, concentrated, and triturated in EtOAc/MeOH give Int. 1AC5 methyl 4-(1-((4-bromo-1-methyl-1H-pyrrolo[2,3-b]pyridin-2-yl)methyl) piperidin-4-yl)benzoate. Int. 1AC5 (0.4 g) and NaOH (0.18 g) were stirred ON in THE/MeOH (2:1, 9 mL), concentrated, and triturated in dilute aq. HCl to give Int. 1AC4 4-[1-[(4-bromo-1-methyl-pyrrolo[2,3-b]pyridin-2-yl)methyl]-4-piperidyl]-benzoic acid (0.38 g). Int. 1AC4 (0.5 g) and CDI (0.7 g) were stirred 2 h in DMF/Et₃N (30:1, 7.2 mL). 25% aq. NH₃ (1.51 mL) was added followed by water to precipitate a solid that was stirred ON in 1,1-dimethoxy-N,N-dimethylmethanamine (10 mL) to afford Int. 1AC3 4-(1-((4-bromo-1-methyl-1H-pyrrolo[2,3-b]pyridin-2-yl)methyl) piperidin-4-yl)benzamide (0.35 g).

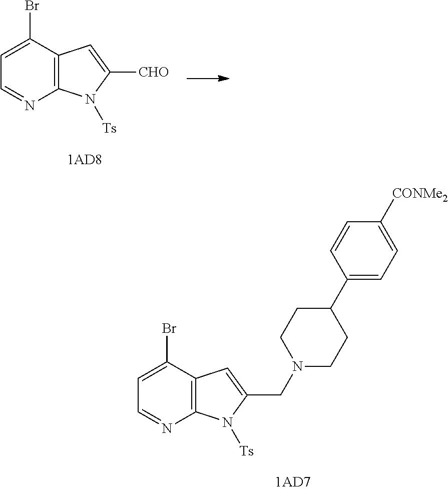

4-Bromo-1-tosyl-1/-pyrrolo[2,3-b]pyridine (1.0 g) was stirred 1 h in 0.6M LDA in THE (14.2 mL) at −78° C. DMF (1.3 mL) was added and stirring continued 2 h at −78° C. The OL (sat. aq. NH₄Cl/E10Ac) was dried, concentrated, and purified by FC (hexane/EtOAc 1:0 to 9:1) to give Int. 1AD8 4-bromo-1-(p-tolylsulfonyl)-pyrrolo[2,3-b]-pyridine-2-carbaldehyde (0.6 g). N,N-Dimethyl-4-(4-piperidinyl)-benzamide (1.4 g, HCl salt) and Int. 1AD8 (1.3 g) were stirred ON in DCE/DIPEA (20:1, 21 mL) at 0° C. to RT. STAB (1.1 g) was stirring continued 2 h at 0° C. to RT. The OL (water/DCM) was dried, concentrated, and purified by FC (DCM/MeOH 9:1) to give Int. 1AD7 4-[1-[4-bromo-1-(p-tolyl-sulfonyl)-pyrrolo[2,3-b]pyridin-2-yl]methyl]-4-piperidyl]-N,N-dimethyl-benzamide (2.3 g).

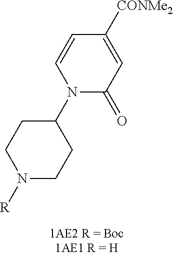

KOtBu (37 mg), tert-butyl 4-(p-tolylsulfonyloxy) piperidine-1-carboxylate (0.16 g), K₂CO₃ (83 mg), and N,N-dimethyl-2-oxo-1H-pyridine-4-carboxamide (50 mg) were degassed in DME (5 mL) and stirred ON at 120° C. The OL (water/MeOH/DCM) was dried and concentrated to give Int. 1AE2 tert-butyl 4-[4-(dimethyl-carbamoyl)-2-oxo-1-pyridyl]-piperidine-1-carboxylate. Int. 1AE2 (0.25 g) was stirred in DCM/TFA (8.6:1, 5.6 mL). The mixture was concentrated to give Int. 1AE1 N,N-Dimethyl-2-oxo-1-(piperidin-4-yl)-1,2-dihydropyridine-4-carboxamide (0.15 mg, TFA salt).

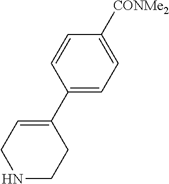

4-Bromo-N,N-dimethylbenzamide (25 g), tert-butyl 4-(4,4,5,5-tetramethyl-1,3,2-dioxaborolan-2-yl)-3,6-dihydropyridine-1(2H)-carboxylate (40.7 g), Na₂CO₃ (34.5 g), and PdDPPFCl₂-DCM (2.24 g) were degassed in dioxane/water (7.7:1, 260 mL) and stirred at 90° C. ON and filtered. The OL (water/EtOAc) was washed with brine, dried, concentrated, and purified by FC (heptane/EtOAc 55:45) to give tert-butyl 4-(4-(dimethyl-carbamoyl)-phenyl)-3,6-dihydropyridine-1(2H)-carboxylate (36.0 g). 5.0 g of this material was stirred ON in DCM/TFA (6.9:1, 46 mL) and concentrated. The OL (sat. aq. NaHCO/10% MeOH in DCM) was washed with brine, dried, concentrated, and triturated in Et₂O to give Int. 1AE7 4-(3,6-dihydro-2H-pyridin-4-yl)-N,N-dimethyl-benzamide (2.80 g).

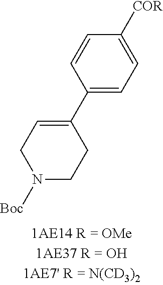

Methyl 4-bromobenzoic acid ester (10 g) tert-butyl 4-(4,4,5,5-tetramethyl-1,3,2-dioxaborolan-2-yl)-3,6-dihydropyridine-1(2H)-carboxylate (14.4 g), NaHCO₃ (11.7 g), and PdDPPFCl₂-DCM (1.2 g) were degassed in dioxane (300 mL) and stirred at 100° C. ON. The OL (water/EtOAc) was dried, concentrated, and purified by FC (hexane/EtOAc 9:1) to give Int. 1AE14 tert-butyl 4-(4-(methoxycarbonyl)phenyl)-3,6-dihydropyridine-1(2H)-carboxylate (11 g). 4.5 g of this material and LiOH—H₂O (3.0 g) were stirred 4 h in MeOH/THF/water (4:2:1, 35 mL) at 0° C. to RT. The mixture was partially concentrated and diluted with aq. citric acid to precipitate Int. 1AE37 4-(1-(tert-butoxycarbonyl)-1,2,3,6-tetrahydropyridin-4-yl)benzoic acid (3.9 g). Int. 1AE37 (4.0 g), HATU 7.52 g), and HN(CD₃)₂ (1.73 g, HCl salt) were stirred ON in DMF/DIPEA (2.5:1, 42 mL). The OL (EtOAc/water) was washed with brine, dried, concentrated, and purified by FC (hexane/EtOAc 1:4) to give tert-butyl 4-[4-[bis-(trideuteriomethyl)-carbamoyl]phenyl]-3,6-dihydro-2H-pyridine-1-carboxylate (3.5 g). 2.5 g of this material was stirred ON in DCM/TFA (1:1, 30 mL) at 0° C. to RT and concentrated. The OL (aq. NaHCO//10% MeOH in DCM) was dried, concentrated, and triturated in Et₂O/pentane to give Int. 1AE7′ N,N-Bis (methyl-d₃)-4-(1,2,3,6-tetrahydropyridin-4-yl)benzamide (1.7 g).

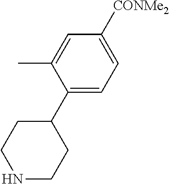

tert-Butyl 4-(4,4,5,5-tetramethyl-1,3,2-dioxaborolan-2-yl)-3,6-dihydropyridine-1(2H)-carboxylate (33.7 g), 4-bromo-N,N,3-trimethylbenzamide (20.0 g), Na₂CO₃ (21.4 g), and PdDPPFCl₂-DCM (1.48 g) were degassed in dioxane/water (9:1, 200 mL) and stirred ON at 110° C. The mixture was filtered, concentrated, and purified by FC (heptane/EtOAc 1:1) to give tert-butyl 4-[4-(dimethylcarbamoyl)-2-methyl-phenyl]-3,6-dihydro-2H-pyridine-1-carboxylate (25 g). 15 g of this material was hydrogenated ON using 10% Pd/C (5.0 g) in MeOH (200 mL), filtered, and concentrated to give tert-butyl 4-[4-(dimethylcarbamoyl)-2-methyl-phenyl]-piperidine-1-carboxylate (14.0 g). 10 g of this material was stirred ON in DCM/TFA (1:1, 200 mL) at 0° C. to RT, concentrated, and triturated in Et₂O to give Int. 1AE8 N,N,3-trimethyl-(4-piperidin-4-yl)benzamide (9.2 g, TFA salt).

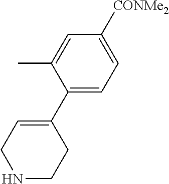

tert-Butyl 4-[4-(dimethylcarbamoyl)-2-methyl-phenyl]-3,6-dihydro-2H-pyridine-1-carboxylate (4.0 g) was stirred 0.5 h in 4M HCl in dioxane (40 mL), concentrated, and triturated in Et₂O to give Int. 1AE8′ N,N,3-trimethyl-4-(1,2,3,6-tetrahydropyridin-4-yl)benzamide (1.06 g, HCl salt).

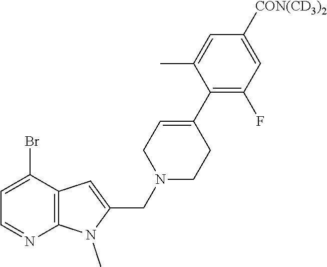

Ints. 1J1/1 (0.70 g/0.49 g, HCl salt) and KI (0.11 g) were stirred ON in DMF/DIPEA (8.3:1, 11.2 mL) at 0° C. to RT. The OL (EtOAc/water) was washed with brine, dried, concentrated, and purified by FC (EtOAc/hexane 7:3) to give Int. 1AE9 4-(1-((4-bromo-1-methyl-1H-pyrrolo[2,3-b]pyridin-2-yl)methyl)-1,2,3,6-tetrahydropyridin-4-yl)-3-fluoro-5-methyl-N,N-bis(methyl-d₃) benzamide (0.45 g).

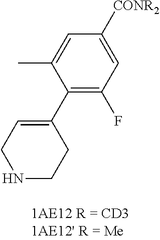

Methyl 4-bromo-3-fluoro-5-methyl-benzoate (20 g), tert-butyl 4-(4,4,5,5-tetramethyl-1,3,2-dioxaborolan-2-yl)-3,6-dihydropyridine-1(2H)-carboxylate (27.5 g), K₂CO₃ (44.8 g), and PdDPPFCl₂-DCM (3.31 g) were degassed in dioxane/water (4:1, 0.5 L) and stirred at 100° C. ON. The OL (water/EtOAc) was washed with brine, dried, concentrated, and purified by FC (heptane to EtOAc) to give tert-butyl 4-(2-fluoro-4-methoxy-carbonyl-6-methyl-phenyl)-3,6-dihydro-2H-pyridine-1-carboxylate (15.5 g). This material and LiOH—H₂O (3.72 g) were stirred ON in MeOH/water (1:1, 0.3 L) and acidified to precipitate 4-(1-tert-butoxycarbonyl-3,6-dihydro-2H-pyridin-4-yl)-3-fluoro-5-methyl-benzoic acid (11.8 g). 5.0 g of this material, HN(CD₃)₂ (1.44 g, HCl salt), and HATU (6.8 g) were stirred ON in DMF/DIPEA (6.4:1, 58 mL), concentrated, and purified by FC (hexane to EtOAc) to give tert-butyl 4-[4-[bis(trideuterio-methyl)carbamoyl]-2-fluoro-6-methyl-phenyl]-3,6-dihydro-2H-pyridine-1-carboxylate (5.1 g). This material was stirred 1 h in HFIP/10 M aq. HCl (31:1, 52 mL) and diluted with MTBE to precipitate Int. 1AE12 3-Fluoro-5-methyl-N,N-bis(methyl-d₃)-4-(1,2,3,6-tetrahydropyridin-4-yl)benzamide (3.45 g, HCl salt). Int. 1AE12′ 3-fluoro-5-methyl-N,N-bis(methyl)-4-(1,2,3,6-tetrahydropyridin-4-yl)benzamide was prepared in a similar manner from HN(CH₃)₂.

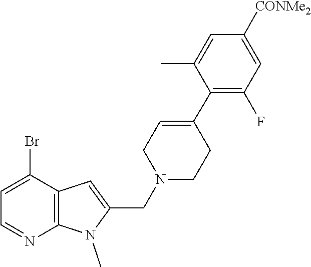

Int. 1J3 (0.50 g) was stirred 1 h in DCM (20 mL) and MsCl (0.8 mL) at 0° C. The OL (water/DCM) was washed with sat. aq. NaHCO₃ and concentrated to give (4-bromo-1-methyl-pyrrolo[2,3-b]pyridin-2-yl)methyl methanesulfonate (0.52 g). 0.5 g of this material, KI (0.13 g), and Int. 1AE12′ (0.51 g) were stirred ON in DMF/DIPEA (14:1, 21.5 mL) at 0° C. to RT. The OL (water/EtOAc) was dried, concentrated, and purified by FC (hexane to EtOAc) to give Int. 1AE13 4-(1-((4-Bromo-1-methyl-1H-pyrrolo[2,3-b]pyridin-2-yl)methyl)-1,2,3,6-tetrahydropyridin-4-yl)-3-fluoro-N,N,5-trimethyl-benzamide (0.52 g).

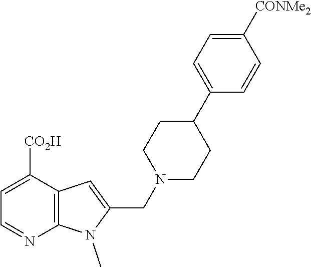

Int. 1AB1 (0.15 g), Mo(CO)₆ (87 mg), Pd₂dba₃ (8 mg), and XantPhos (10 mg) were degassed in EtOH (5 mL) and stirred 0.5 h at 120° C. under MW conditions. The mixture was filtered and HPLC-purified give ethyl 2-((4-(4-(dimethylcarbamoyl)phenyl) piperidin-1-yl)methyl)-1-methyl-1H-pyrrolo[2,3-b]pyridine-4-carboxylate (0.25 g). This material and LiOH (69 mg) were stirred in water/EtOH (2:1, 1.5 mL), concentrated, and HPLC-purified to give Int. 1AE15 2-((4-(4-(Dimethylcarbamoyl)phenyl) piperidin-1-yl)methyl)-1-methyl-1H-pyrrolo[2,3-b]pyridine-4-carboxylic acid (0.15 g).

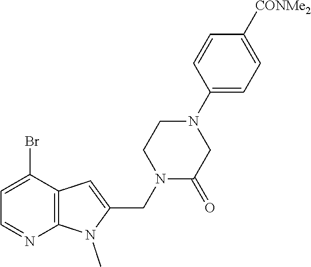

Piperazin-2-one (1.0 g), 4-bromo-N,N-dimethylbenzamide (1.14 g), K₃PO₄ (6.4 g), Pd (OAc) 2 (0.22 g), and RuPhos (0.93 g) were degassed in tert-butyl alcohol (15 mL) and stirred ON at 80-90° C. The mixture was filtered, concentrated, and purified by FC (DCM/MeOH 9:1) to give N,N-dimethyl-4-(3-oxopiperazin-1-yl)benzamide (0.70 g). 0.25 g of this material and NaH (81 mg) were stirred 1 h in THF/DMF (10:1, 5.5 mL) at 0° C. to RT. Int. 1J1 (0.25 g) was added and stirring continued ON at 0° C. to RT. The OL (10% MeOH in DCM/water) was dried, concentrated, and purified by FC (DCM/MeOH 9:1) to give Int. 1AE16 4-(4-((4-Bromo-1-methyl-1H-pyrrolo[2,3-b]pyridin-2-yl)methyl)-3-oxopiperazin-1-yl)-N,N-dimethylbenzamide (0.20 g).

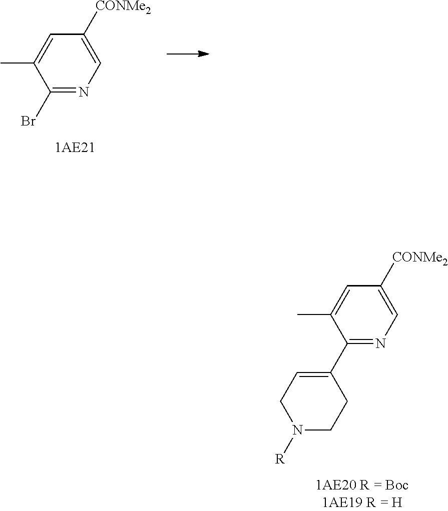

6-Bromo-5-methyl-pyridine-3-carboxylic acid (4.5 g), HATU (9.5 g), and HN(CH₃)₂ (8.4 g, HCl salt) were stirred ON in DMF/DIPEA (2.5:1, 45 mL) at 0° C. to RT. The OL (water/EtOAc) was dried, concentrated and purified by FC (hexane/EtOAc 3:7) to give Int. 1AE21 6-bromo-N,N,5-trimethylnicotinamide (4.2 g). 3.2 g of this material, NaHCO₃ (3.9 g), tert-butyl 4-(4,4,5,5-tetramethyl-1,3,2-dioxaborolan-2-yl)-3,6-dihydro-pyridine-1(2H)-carboxylate (6.1 g), and PdDPPFCl₂-DCM (0.27 g) were degassed in dioxane/water (3.7:1, 14 mL) and stirred ON at 110° C. The mixture was filtered, concentrated, and purified by FC (hexane/EtOAc 1:4) to give Int. 1AE20 tert-butyl 5-(dimethylcarbamoyl)-3-methyl-3′, 6′-dihydro-[2,4′-bipyridine]-1′ (2′H)-carboxylate (4.0 g). 0.25 g of this material was stirred ON in DCM/TFA (8.4:1, 5.6 mL) at 0° C. to RT, concentrated, and triturated in Et₂O/pentane to give Int. 1AE19 N,N,5-trimethyl-6-(1,2,3,6-tetrahydro-pyridin-4-yl)pyridine-3-carboxamide (0.28 g, TFA salt).

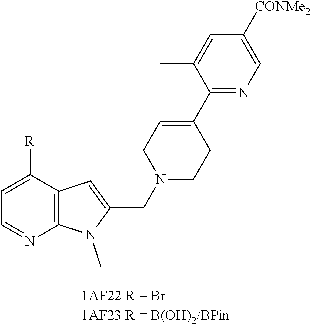

Ints. 1AE19/1J1 (0.5 g/0.5 g) were stirred ON ACN/Et₃N (18:1, 26 mL), concentrated, and HPLC-purified to give Int. 1AE22 6-[1-[(4-bromo-1-methyl-pyrrolo[2,3-b]pyridin-2-yl)methyl]-3,6-dihydro-2H-pyridin-4-yl]-N,N,5-trimethyl-pyridine-3-carboxamide (0.3 g). Int. 1AE22 (3 g), B₂Pin₂ (5.0 g), KOAc (5.0 g), and PdDPPFCl₂-DCM (0.05 g) were degassed in dioxane (25 mL) and stirred ON at 80° C. The OL (water/EtOAc) was dried and concentrated to give Int. 1AE23 (2- ((5-(dimethylcarbamoyl)-3-methyl-3′, 6′-dihydro-[2,4′-bipyridin]-1′ (2′H)-yl)methyl)-1-methyl-1H-pyrrolo[2,3-b]pyridin-4-yl) boronic acid/N,N,3-trimethyl-1′-((1-methyl-4-(4,4,5,5-tetramethyl-1,3,2-dioxaborolan-2-yl)-1H-pyrrolo[2,3-b]pyridin-2-yl)methyl)-1′,2′,3′,6′-tetrahydro-[2,4′-bipyridine]-5-carboxamide (1.0 g).

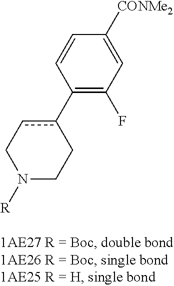

4-Bromo-3-fluoro-N,N-dimethyl-benzamide (1.0 g), tert-butyl 4-(4,4,5,5-tetramethyl-1,3,2-dioxaborolan-2-yl)-3,6-dihydro-2H-pyridine-1-carboxylate (1.51 g), Na₂CO₃ (1.72 g), and PdDPPFCl₂-DCM (0.17 g) were degassed in dioxane/water (4:1, 10 mL) and stirred at 100° C. ON. The OL (water/EtOAc) was washed with brine, dried, concentrated, and purified by FC (hexane to EtOAc) to give Int. 1AE27 tert-butyl 4-[4-(dimethyl-carbamoyl)-2-fluoro-phenyl]-3,6-dihydro-2H-pyridine-1-carboxylate (1.2 g). This material was hydrogenated ON using 10% Pd/C (40 mg) in MeOH (10 mL) under an atmosphere of H2 in a sealed tube, filtered, and concentrated to give Int. 1AE26 tert-butyl 4-[4-(dimethylcarba-moyl)-2-fluoro-phenyl]piperidine-1-carboxylate (0.60 g) that was stirred in 4M HCl in dioxane (10 mL) ON, concentrated, and HPLC-purified to give Int. 1AE25 3-fluoro-N,N-dimethyl-4-(piperidin-4-yl)benzamide (63 mg).

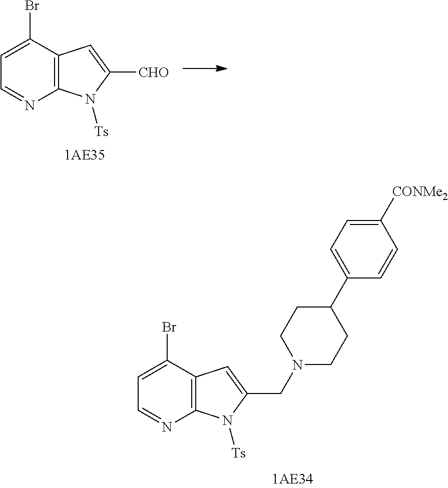

4-Bromo-1-tosyl-1H-pyrrolo[2,3-b]pyridine (1.0 g) was stirred 1 h in 0.6 M LDA in THF (14 mL) at −78° C. DMF (1.3 mL) was added and stirring continued 2 h at −78° C. The OL (sat. aq. NH₄Cl/EtOAc) was dried, concentrated, and purified by FC (hexane/EtOAc 9:1) to give Int. 1AE35 4-bromo-1-(p-tolyl-sulfonyl) pyrrolo[2,3-b]pyridine-2-carbaldehyde (0.6 g). Int. 1A35 (1.5 g) and N,N-dimethyl-4-(piperidin-4-yl)benzamide (1.6 g, HCl salt) were stirred ON in DCM/DIPEA (5:1, 24 mL). STAB (2.5 g) was added and stirring continued 2 h at 0° C. to RT. The OL (DCM/water) was dried, concentrated, and purified by FC (DCM/MeOH 9:1) to give Int. 1AE34 4-[1-[4-bromo-1-(p-tolylsulfonyl)-pyrrolo[2,3-b]pyridin-2-yl]methyl]-4-piperidyl]-N,N-dimethyl-benzamide (1.0 g).

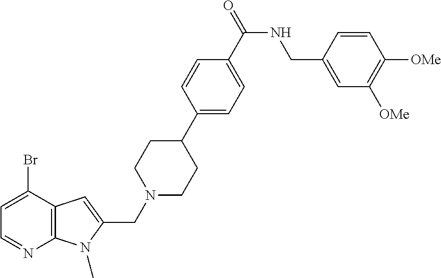 [SMILES: COc1ccc(CNC(=O)c2ccc(C3CCN(Cc4cc5c(Br)ccnc5n4C)CC3)cc2)cc1OC]

Int. 1AC4 (0.10 g), HATU (164 mg), and (3,4-dimethoxybenzyl) (methyl)-amine (58 mg) were stirred 3 h in DMF/DIPEA (25:1, 3.1 mL). The OL (sat. aq. NaHCO₃/DCM) was dried, concentrated, and purified by FC (DCM to DCM/MeOH 93:7) to give Int. 1AE36 4-(1-((4-bromo-1-methyl-1H-pyrrolo-[2,3-b]pyridin-2-yl)methyl) piperidin-4-yl)-N-(3,4-dimethoxybenzyl)-N-methylbenzamide (0.12 g).

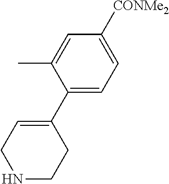

tert-Butyl 4-[4-(dimethylcarbamoyl)-2-methyl-phenyl]-3,6-dihydro-2H-pyridine-1-carboxylate (4.0 g) was stirred 0.5 h in 4M HCl in dioxane (40 mL), concentrated, and triturated in Et₂O to give Int. 1AE38 N,N,3-trimethyl-4-(1,2,3,6-tetrahydropyridin-4-yl)benzamide (1.06 g, HCl salt).

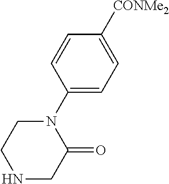

tert-Butyl 3-oxopiperazine-1-carboxylate (0.50 g), 4-bromo-N,N-dimethylbenzamide (0.68 g), K₃PO₄ (1.06 g), CuI (71 mg), DMDCH (36 mg), were degassed in dioxane (10 mL) and was stirred at 100° C. ON. The OL (water/EtOAc) was dried, concentrated, and purified by FC (EtOAc/MeOH 1:0 to 9:1) to give tert-butyl 4-(4-(dimethylcarbamoyl)-phenyl)-3-oxopiperazine-1-carboxylate (0.80 g). 0.23 g of this material was stirred 1 h in DCM/TFA (1:1, 6 mL) and concentrated to give Int. 1AE39 N,N-Dimethyl-4-(2-oxo-piperazin-1-yl)benzamide (0.24 g, TFA salt).

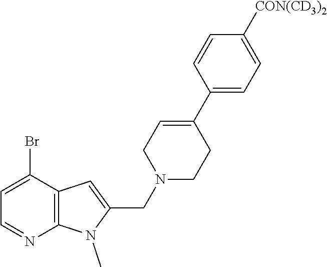

Int. 1J1/1AE7′ (0.33 g/0.30 g), and KI (21 mg) were stirred ON in DMF/DIPEA (7.3:1, 9 mL) at 0° C. to RT. The OL (water/EtOAc) was dried, concentrated, and purified by FC (hexane to EtOAc) to give Int. 1AF1 4-(1-((4-Bromo-1-methyl-1H-pyrrolo[2,3-b]pyridin-2-yl)methyl)-1,2,3,6-tetrahydropyridin-4-yl)-N,N-bis(methyl-d₃) benzamide (0.20 g).

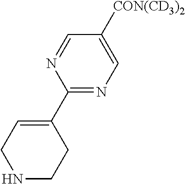

Methyl 2-bromopyrimidine-5-carboxylate (10.0 g), tert-butyl 4-(4,4,5,5-tetramethyl-1,3,2-dioxaborolan-2-yl)-3,6-dihydropyridine-1(2H)-carboxylate (15.6 g), K₂CO₃ (25.5 g), and PdDPPFCl₂-DCM (1.88 g) were degassed in dioxane/water (4:1, 0.25 L) and stirred ON at 100° C. The OL (water/EtOAc) was washed with brine, dried, concentrated, and purified by FC (hexane to EtOAc) to give methyl 2-(1-tert-butoxycarbonyl-3,6-dihydro-2H-pyridin-4-yl)pyrimidine-5-carboxylate (14.7 g). This material and LiOH—H₂O (2.15 g) were stirred ON in MeOH/water (1:1, 200 mL) and acidified to precipitate 2-(1-tert-butoxy-carbonyl-3,6-dihydro-2H-pyridin-4-yl)pyrimidine-5-carboxylic acid (8.0 g). 4.0 g of this material, HN(CD₃)₂ (1.21 g, HCl salt), and HATU (5.27 g) were stirred in DMF/DIPEA (3:1, 27 mL), concentrated, and purified by FC (hexane to EtOAc) to give tert-butyl 4-[5-[bis(tri-deuteriomethyl)carbamoyl] pyrimidin-2-yl]-3,6-dihydro-2H-pyridine-1-carboxylate (2.20 g). This material was stirred in 1 h HFIP/10M aq. HCl (30:1, 23 mL), concentrated, and triturated in MTBE to give Int. 1AF2 N,N-bis(methyl-d₃)-2-(1,2,3,6-tetrahydropyridin-4-yl)pyrimidine-5-carboxamide (1.40 g, HCl salt).

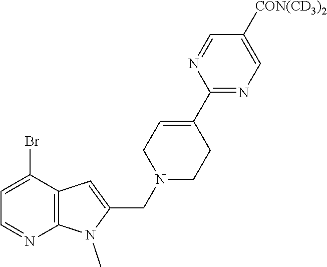

Ints. 1AF2/1J1 (0.5 g/0.5 g) were stirred in ACN/Et₃N (19:1, 26 mL), concentrated, and HPLC-purified to give Int. 1AF3 2-[1-[(4-Bromo-1-methyl-pyrrolo[2,3-b]pyridin-2-yl)methyl]-3,6-dihydro-2H-pyridin-4-yl]-N,N-bis(trideuteriomethyl)pyrimidine-5-carboxamide (0.3 g).

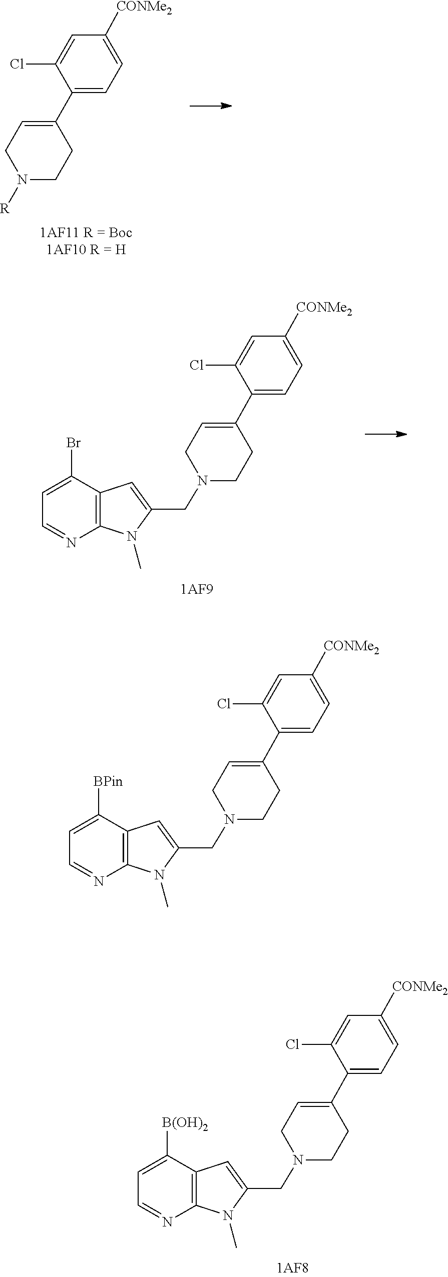

4-Bromo-3-fluoro-N,N-dimethyl-benzamide (1 g), tert-butyl 4-(4,4,5,5-tetramethyl-1,3,2-dioxaborolan-2-yl)-3,6-dihydropyridine-1(2H)-carboxylate (1.5 g), Na₂CO₃ (1.7 g), PdPDDFCl₂-DCM (0.17 g) were deassed in dioxane/water (4:1, 10 mL) and stirred ON at 100° C. The OL (water/EtOAc) was washed with brine, dried, concentrated, and purified by FC (hexane to EtOAc) to give Int. 1AF11 tert-butyl 4-[4-(dimethylcarbamoyl)-2-fluoro-phenyl]-3,6-dihydro-2H-pyridine-1-carboxylate (1.2 g). Int. 1AF11 (0.6 g) was stirred ON in 4M HCl in dioxane (10 mL). The residue after concentration was HPLC-purified to give Int. 1AF10 3-fluoro-N,N-dimethyl-4-(1,2,3,6-tetrahydropyridin-4-yl)benzamide (0.22 g). Ints. 1AF10/1J1 (0.5 g/0.5 g) were stirred ON in ACN/Et₃N (19:1, 26 mL), concentrated, and HPLC-purified to give Int. 1AF9 4-[1-[(4-bromo-1-methyl-pyrrolo[2,3-b]pyridin-2-yl)-methyl]-3,6-dihydro-2H-pyridin-4-yl]-N,N,3,5-tetramethyl-benzamide (0.3 g). Int. 1AF9 (3 g), B₂pin₂ (5 g), KOAc (5 g), and PdDPPFCl₂-DCM (50 mg) were degassed in dioxane and stirred ON at 80° C. The OL (EtOAc/water) was dried and concentrated to give Int. 1AF8 3-chloro-N,N-dimethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide/(2-((4-(2-chloro-4-(dimethylcarbamoyl)phenyl)-3,6-dihydropyridin-1(2H)-yl)methyl)-1-methyl-1H-pyrrolo[2,3-b]pyridin-4-yl) boronic acid (1 g).

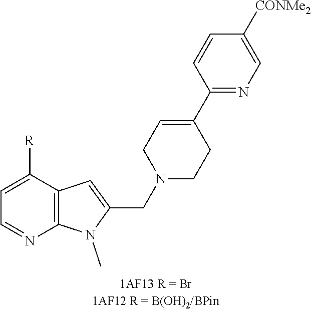

Ints. 1S5/1J1 (0.5 g/0.5 g, as the HCl salt) were stirred ON in ACN/Et₃N (19:1, 26 mL), concentrated, and HPLC-purified to give Int. 1AF13 6-[1-[(4-bromo-1-methyl-pyrrolo-[2,3-b]pyridin-2-yl)methyl]-3,6-dihydro-2H-pyridin-4-yl]-N,N-dimethyl-pyridine-3-carboxamide (0.3 g). Int. 1AF13 (3.0 g), KOAc (5.0 g), B₂Pin₂ (5.0 g), and PdDPPFCl₂-DCM (50 mg) were degassed in dioxane (25 mL) and stirred at 80° C. ON. The OL (water/EtOAc) was concentrated to give Example 1AF12 N,N-Dimethyl-1′-((1-methyl-4-(4,4,5,5-tetramethyl-1,3,2-dioxaborolan-2-yl)-1H-pyrrolo[2,3-b]pyridin-2-yl)methyl)-1′,2′,3′,6′-tetrahydro-[2,4′-bipyridine]-5-carboxamide/(2-((5-(dimethylcarbamoyl)-3′, 6′-dihydro-[2,4′-bipyridin]-1′ (2′H)-yl)methyl)-1-methyl-1H-pyrrolo[2,3-b]pyridin-4-yl) boronic acid (1.0 g).

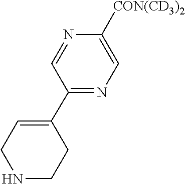

Methyl 5-bromopyrazine-2-carboxylate (11.0 g), tert-butyl 4-(4,4,5,5-tetramethyl-1,3,2-dioxaborolan-2-yl)-3,6-dihydropyridine-1(2H)-carboxylate (17.2 g), K₂CO₃ (28.0 g), PdPDDFCl₂-DCM (2.07 g) were deassed in dioxane/water (5:1, 250 mL) and stirred ON at 100° C. The aq. layer (water/EtOAc) was acidified to precipitate 5-(1-tert-butoxy-carbonyl-3,6-dihydro-2H-pyridin-4-yl) pyrazine-2-carboxylic acid (12.0 g). 4.5 g of this material, HN(CD₃)₂ (1.40 g, HCl salt), and HATU (6.20 g) were stirred ON in DMF/DIPEA (2.6:1, 28 mL), concentrated, and purified by FC (hexane to EtOAc) to give tert-butyl 4-[5-[bis(trideuteriomethyl)-carbamoyl] pyrazin-2-yl]-3,6-dihydro-2H-pyridine-1-carboxylate (2.0 g). This material was stirred in 1 h HFIP/10M aq. HCl (29:1, 21 mL), concentrated, and triturated in MTBE to give Int. 1AF14 5-(1,2,3,6-Tetrahydropyridin-4-yl)-N,N-bis(trideuteriomethyl) pyrazine-2-carboxamide (0.88 g, HCl salt).

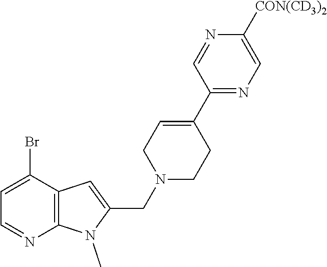

Ints. 1AF14/1J1 (0.5 g/0.5 g), and Et₃N (1.2 mL) were stirred ON in ACN (25 mL), concentrated, and HPLC-purified to give Int. 1AF15 5-(1-((4-Bromo-1-methyl-1H-pyrrolo[2,3-b]pyridin-2-yl)methyl)-1,2,3,6-tetrahydropyridin-4-yl)-N,N-bis(methyl-d₃) pyrazine-2-carboxamide (0.3 g).

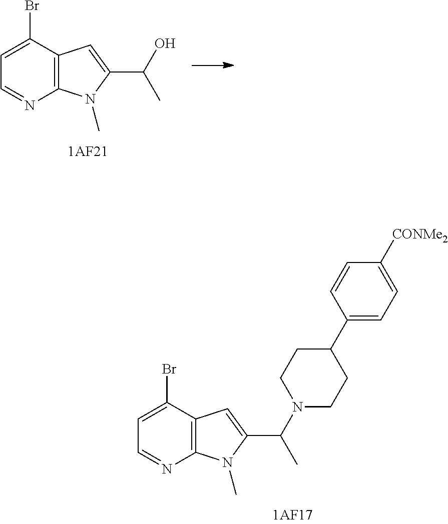

1.0M MeMgBr in THF (185 mL) was added to a solution of Int. 1AB8 (29.5 g) in THF (1.2 L) at 0° C. and stirring continued 3 h. The OL (sat. aq. NH₄Cl/EtOAc) was washed with brine, dried, concentrated and purified by FC (pentane/EtOAc 3:1) to give Int. 1AF21 1-(4-bromo-1-methyl-1H-pyrrolo[2,3-b]pyridin-2-yl) ethan-1-ol (25.5 g). 0.30 g of this material was stirred in toluene/SOCl₂ (12.5:1, 10.8 mL) at 0° C. before stirring 0.5 h at 110° C. The mixture was concentrated. The residue, Cs₂CO₃ (1.15 g), KI (0.98 g), and N,N-dimethyl-4-(4-piperidyl)-benzamide (0.41 g) were stirred ON in ACN (10 mL) at 0° C. to RT. The OL (water/EtOAc) was washed with brine, dried, concentrated, and purified by FC (DCM/MeOH 95:5) to give Int. 1AF17 4-(1-(1-(4-bromo-1-methyl-1H-pyrrolo[2,3-b]pyridin-2-yl)ethyl)-piperidin-4-yl)-N,N-dimethylbenzamide (0.28 g).

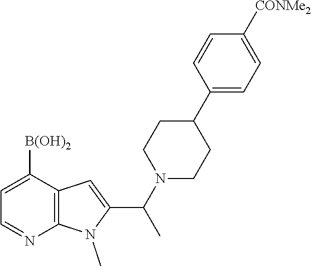

Int. 1AF17 (0.70 g), B₂Pin₂ (0.51 g), KOAc (0.4 g), and Pd(PPh₃)₂Cl₂ (0.1 g) were degassed in THF (50 mL) and stirred ON at 100° C., filtered, concentrated, and triturated in pentane to give Int. 1AF24 (2-(1-(4-(4-(dimethyl-carbamoyl)phenyl) piperidin-1-yl)ethyl)-1-methyl-1H-pyrrolo[2,3-b]pyridin-4-yl) boronic acid (0.85 g).

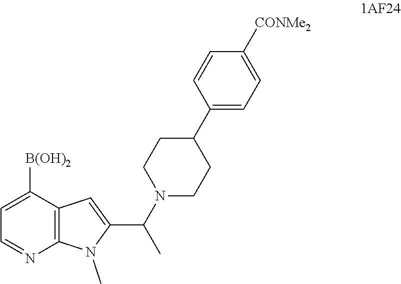

Int. 1AF17 (0.70 g), B₂Pin₂ (0.51 g), KOAc (0.4 g), and Pd(PPh₃)₂Cl₂ (0.1 g) were degassed in THF (50 mL) and stirred ON at 100° C., filtered, concentrated, and triturated in pentane to give Int. 1AF24 (2-(1-(4-(4-(dimethyl-carbamoyl)phenyl) piperidin-1-yl)ethyl)-1-methyl-1H-pyrrolo[2,3-b]pyridin-4-yl) boronic acid (0.85 g).

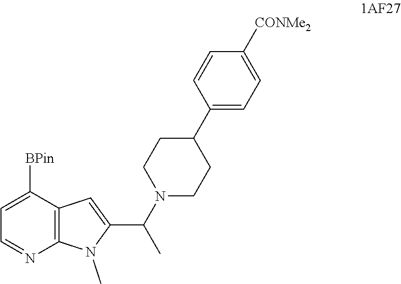

Int. 1AF17 (0.70 g), B₂Pin₂ (38 mg), KOAc (45 mg), and PdDPPFCl₂-DCM (25 mg) were degassed in dioxane (10 mL) and stirred ON at 110° C. The mixture was filtered, concentrated, and purified by FC (pentane/EtOAc 95:5) to give Int. 1AF27 N,N-dimethyl-4-(1-(1-(1-methyl-4-(4,4,5,5-tetramethyl-1,3,2-dioxaborolan-2-yl)-1H-pyrrolo-[2,3-b]pyridin-2-yl)ethyl) piperidin-4-yl)benzamide (0.85 g).

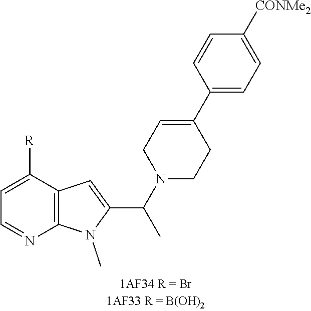

1-(4-Bromo-1-methyl-1H-pyrrolo[2,3-b]pyridin-2-yl) ethan-1-ol (0.20 g) was stirred 1.5 h in toluene/SOCl₂ (29:1, 5.2 mL) at 0° C. to 110° C. and concentrated. The residue, KI (10 mg), and Int. 1AF5 (0.23 g) were stirred ON in DMF/DIPEA (2.5:1, 2.8 mL) at 0° C. to RT. The OL (water/EtOAc) was washed with brine, dried, concentrated, and purified by FC (hexane/EtOAc 9:1 to 85:15) to give Int. 1AF34 4-[1-[1-(4-bromo-1-methyl-pyrrolo[2,3-b]pyridin-2-yl)ethyl]-3,6-dihydro-2H-pyridin-4-yl]-N,N-dimethyl-benzamide (80 mg). Int. 1AF34 (0.32 g), B₂Pin₂ (24 mg), KOAc (25 mg), and PdCl₂ (PPh₃)₂ (48 mg) were degassed in THF (5 mL) and stirred ON at 90° C. The mixture was filtered, concentrated, and purified by FC (pentane/EtOAc 95:5) to give Int. 1AF33 (2-(1-(4-(4-(dimethylcarbamoyl)-phenyl)-3,6-dihydropyridin-1(2H)-yl)ethyl)-1-methyl-1H-pyrrolo[2,3-b]pyridin-4-yl)-boronic acid (0.50 g).

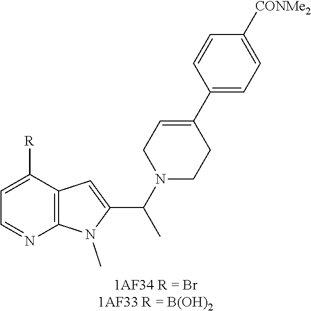

1-(4-Bromo-1-methyl-1H-pyrrolo[2,3-b]pyridin-2-yl) ethan-1-ol (0.20 g) was stirred 1.5 h in toluene/SOCl₂ (29:1, 5.2 mL) at 0° C. to 110° C. and concentrated. The residue, KI (10 mg), and Int. 1AF5 (0.23 g) were stirred ON in DMF/DIPEA (2.5:1, 2.8 mL) at 0° C. to RT. The OL (water/EtOAc) was washed with brine, dried, concentrated, and purified by FC (hexane/EtOAc 9:1 to 85:15) to give Int. 1AF34 4-[1-[1-(4-bromo-1-methyl-pyrrolo-[2,3-b]pyridin-2-yl)ethyl]-3,6-dihydro-2H-pyridin-4-yl]-N,N-dimethyl-benzamide (80 mg). Int. 1AF34 (0.32 g), B₂Pin₂ (24 mg), KOAc (25 mg), and PdCl₂ (PPh₃)₂ (48 mg) were degassed in THF (5 mL) and stirred ON at 90° C. The mixture was filtered, concentrated, and purified by FC (pentane/EtOAc 95:5) to give Int. 1AF33 (2-(1-(4-(4-(dimethyl-carbamoyl)-phenyl)-3,6-dihydropyridin-1(2H)-yl)ethyl)-1-methyl-1H-pyrrolo[2,3-b]pyridin-4-yl)-boronic acid (0.50 g).

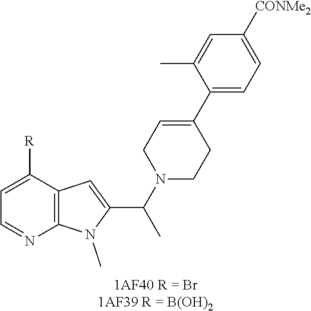

Int. 1AF40 4-[1-[1-(4-bromo-1-methyl-pyrrolo[2,3-b]pyridin-2-yl)ethyl]-3,6-dihydro-2H-pyridin-4-yl]-N,N,3-trimethyl-benzamide (0.15 g) was prepared similarly to Int. 1AF17 from Int. 1AF21 (0.20 g) and Int. 1AE38 (0.37 g, TFA salt). Int. 1AF39 (2-(1-(4-(4-(dimethylcarbamoyl)-2-methylphenyl)-3,6-dihydropyridin-1(2H)-yl)-ethyl)-1-methyl-1H-pyrrolo[2,3-b]pyridin-4-yl) boronic acid (40 mg) was prepared similarly to Int. 1AF33 from Int. 1AF40 (50 mg).

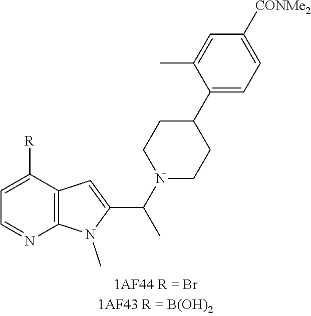

Int. 1AF44 4-[1-[1-(4-bromo-1-methyl-pyrrolo[2,3-b]pyridin-2-yl)ethyl]-4-piperidyl]-N,N,3-trimethyl-benzamide (0.15 g) was prepared similarly to Int. 1AF17 from Ints. 1AF24/1AE38 (0.20 g/0.42 g, TFA salt). Int. 1AF43 (2-(1-(4-(4-(dimethylcarbamoyl)-2-methylphenyl)-3,6-dihydropyridin-1(2H)-yl)ethyl)-1-methyl-1H-pyrrolo[2,3-b]-pyridin-4-yl) boronic acid (0.60 g) was prepared similarly to Int. 1AF39 from Int. 1AF44 (0.50 g).

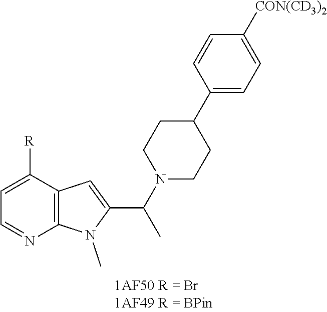

Int. 1AF50 4-[1-[1-(4-bromo-1-methyl-pyrrolo[2,3-b]pyridin-2-yl)ethyl]-4-piperidyl]-N,N-bis(trideuterio-methyl)benzamid (0.65 g) was prepared similarly to Int. 1AF17 from Ints. 1AF21/1D7 (0.35 g/0.53 g, TFA salt). Int. 1AF49 [2-[1-[4-[4-[bis(trideuterio-methyl)carbamoyl]phenyl]-1-piperidyl]ethyl]-1-methyl-pyrrolo[2,3-b]-pyridin-4-yl]-boronic acid (0.41 g) was prepared similarly to Int. 1AF25 from Int. 1AF50 (0.32 g) and B₂Pin₂ (0.34 g).

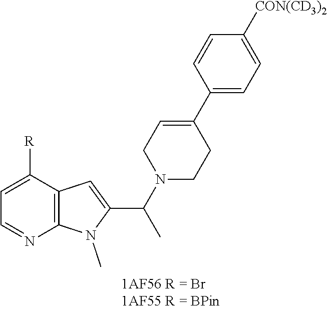

Int. 1AF56 4-(1-(1-(4-bromo-1-methyl-1H-pyrrolo[2,3-b]pyridin-2-yl)ethyl)-1,2,3,6-tetrahydropyridin-4-yl)-N,N-bis(methyl-d₃) benzamide (0.60 g) was prepared similarly to Int. 1AF34 from Int. 1AF21 (0.50 g) and Int. 1AE7′ (0.69 g). Int. 1AF55 [2-[1-[4-[4-[bis(trideuteriomethyl)carbamoyl]phenyl]-3,6-dihydro-2H-pyridin-1-ylethyl]-1-methyl-pyrrolo[2,3-b]pyridin-4-yl]boronic acid (0.55 g) was prepared similarly to Int. 1AF39 from Int. 1AF56 (0.50 g).

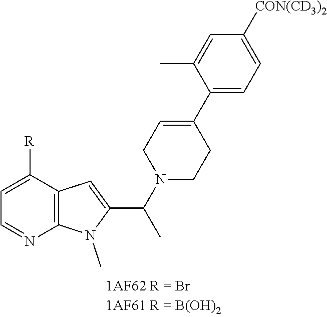

Int. 1AF62 4-(1-(1-(4-bromo-1-methyl-1H-pyrrolo[2,3-b]pyridin-2-yl)ethyl)-1,2,3,6-tetrahydro-pyridin-4-yl)-3-methyl-N,N-bis(methyl-d₃) benzamide (0.46 g) was prepared similarly to Int. 1AF17 from Ints. 1AF24/1S20 (0.50 g/0.70 g). Int. 1AF61 (2-(1-(4-(4-(bis(methyl-d₃)carbamoyl)-2-methylphenyl)-3,6-dihydropyridin-1(2H)-yl)ethyl)-1-methyl-1H-pyrrolo[2,3-b]pyridin-4-yl) boronic acid (0.75 g) was prepared similarly to Int. 1AF55 from B₂Pin₂ (0.52 g) and Int. 1AF62 (0.5 g).

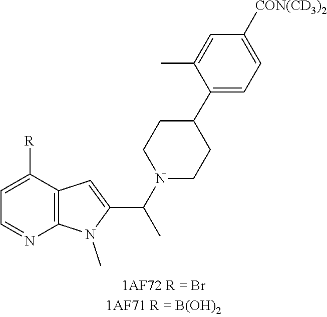

Int. 1AF72 4-(1-(1-(4-bromo-1-methyl-1H-pyrrolo[2,3-b]pyridin-2-yl)ethyl) piperidin-4-yl)-3-methyl-N,N-bis-(methyl-d₃) benzamide (90 mg) was prepared similarly to Int. 1AF17 from Ints. 1AF24/1S22 (0.10 g/0.19 g, TFA salt). Int. 1AF71 (2-(1-(4-(4-(bis(methyl-d₃)carbamoyl)-2-methylphenyl) piperidin-1-yl)ethyl)-1-methyl-1H-pyrrolo[2,3-b]pyridin-4-yl) boronic acid (0.10 g) was prepared similarly to Int. 1AF55 from Int. 1AF72 (90 mg) and B₂Pin₂ (0.12 g).

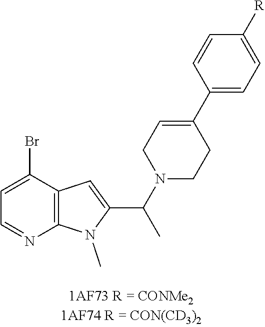

Int. 1AF21 (1.20 g) was stirred 1 h in toluene/SOCl₂ (5.9:1, 23.5 mL) at 0° C. to 110° C. and concentrated. The residue, Cs₂CO₃ (4.60 g), KI (0.39 g), and Int. 1AE7 (1.30 g) were stirred ON in ACN (30 mL) at 0° C. to RT. The OL (water/EtOAc) was washed with brine, dried, concentrated, and purified by FC (hexane/EtOAc 9:1 to 85:15) to give Int. 1AF73 4-[1-[1-(4-bromo-1-methyl-pyrrolo[2,3-b]pyridin-2-yl)ethyl]-3,6-dihydro-2H-pyridin-4-yl]-N,N-dimethyl-benzamide (1.0 g). Int. 1AF74 4-(1-(1-(4-bromo-1-methyl-1H-pyrrolo[2,3-b]pyridin-2-yl)ethyl)-1,2,3,6-tetrahydropyridin-4-yl)-N,N-bis(methyl-d₃) benzamide (0.60 g) was prepared similarly from Ints. 1AE7′/ 1AF21 (0.69 g/0.50 g).

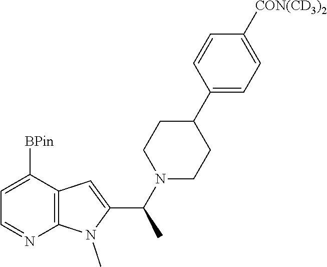

Int. 1AF76 (1.50 g), B₂Pin₂ (1.60 g), KOAc (0.93 g), and PdPDDFCl₂-DCM (0.26 g) were degassed in dioxane (30 mL) and stirred 6 h at 90° C., filtered, concentrated, and triturated in Et₂O to give Int. 1AF75 (S)—N,N-bis(methyl-d₃)-4-(1-(1-(1-methyl-4-(4,4,5,5-tetramethyl-1,3,2-dioxaborolan-2-yl)-1H-pyrrolo[2,3-b]pyridin-2-yl)ethyl)-piperidin-4-yl)benzamide (1.30 g).

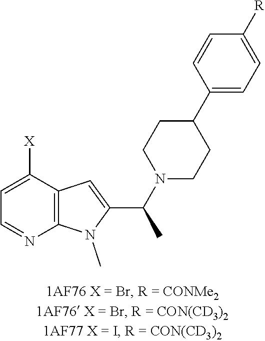

Int. 2C3-6 (0.35 g), HATU (0.3 g), and HN(CD₃)₂ (77 mg, HCl salt) were stirred ON in DMF/DIPEA (14.7:1, 10.7 mL) at 0° C. to RT and diluted with water to precipitate Int. 1AF76 (0.30 g). Int. 1AF76′ (3.5 g) was prepared similarly from Int. 2C3-6 (4.5 g). Int. 1AF76′ (0.10 g), CuI (20 mg), NaI (63 mg), and DMDCH (30 mg) were degassed in dioxane (5 mL) and stirred at 110° C. ON and filtered. The OL (DCM/aq. NH₃) was dried, concentrated, and purified by FC (DCM/MeOH 97:3) to give Int. 1AF77 (S)-4-(1-(1-(4-iodo-1-methyl-1H-pyrrolo[2,3-b]pyridin-2-yl)ethyl) piperidin-4-yl)-N,N-bis(methyl-d₃)-benzamide (50 mg). The absolute configuration of 1AF76 (S)-4-(1-(1-(4-bromo-1-methyl-1H-pyrrolo-[2,3-b]pyridin-2-yl)-ethyl) piperidin-4-yl)-N,N-dimethylbenzamide and 1AF76′ (S)-4-(1-(1-(4-bromo-1-methyl-1H-pyrrolo-[2,3-b]pyridin-2-yl)ethyl)-piperidin-4-yl)-N,N-bis(methyl-d₃) benzamide was determined to S by VCD.

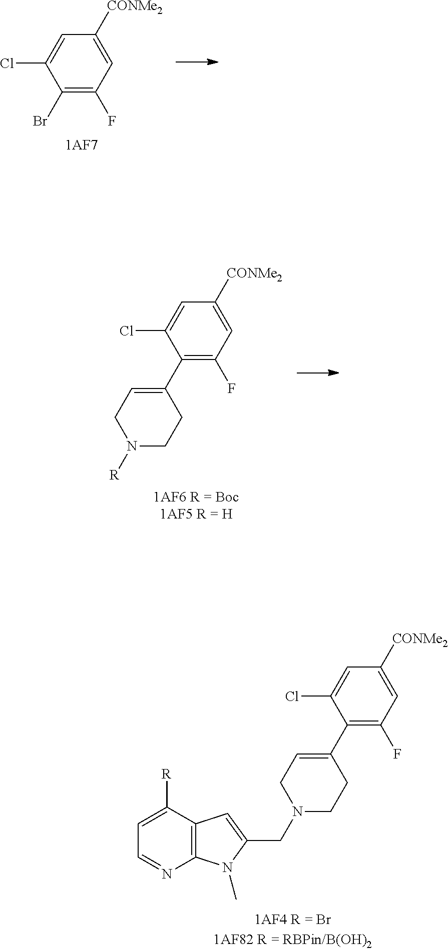

CDI (7.04 g) was added to a solution of 4-bromo-3-fluoro-5-methyl-benzoyl chloride (11.0 g) in DCM (50 mL) at 0° C. and stirring continued 0.5 h. HN(CH₃)₂ (7.1 g, HCl salt) was added before refluxing 5 h. The mixture was washed with water, dried, and concentrated to give Int. 1AF7 4-bromo-3-fluoro-N,N,5-trimethyl-benzamide (12.4 g). This material, tert-butyl 4-(4,4,5,5-tetramethyl-1,3,2-dioxa-borolan-2-yl)-3,6-dihydro-2H-pyridine-1-carboxylate (15.0 g), K₂CO₃ (18.3 g), and PdDPPFCl₂-DCM (0.73 g) were degassed in dioxane (100 mL) and stirred 48 h at 90° C. The OL (water/MTBE) was dried and concentrated to give Int. 1AF6 tert-butyl 4-(2-chloro-4-(dimethylcarbamoyl)-6-fluorophenyl)-3,6-dihydropyridine-1(2H)-carboxylate (11.0 g). Int. 1AF6 (0.6 g) was stirred 0.5 h in THF/4M HCl in dioxane (1:1, 2 mL). The OL (water/EtOAc) was dried and concentrated to give Int. 1AF5 3-chloro-5-fluoro-N,N-dimethyl-4-(1,2,3,6-tetrahydro-pyridin-4-yl)benzamide (0.25 g, HCl salt). This material, K₂CO₃ (0.37 g), and Int. 1J1 (0.23 g) were stirred in DMF (5 mL) ON. The OL (water/MTBE) was layer was dried and concentrated to give Int. 1AF4 4-[1-[(4-bromo-1-methyl-pyrrolo[2,3-b]pyridin-2-yl)-methyl]-3,6-dihydro-2H-pyridin-4-yl]-3-chloro-5-fluoro-N,N-dimethyl-benzamide (0.17 g). This material, B₂Pin₂ (0.17 g), KOAc (0.10 g), and PdDPPFCl₂-DCM (6 mg) were degassed in dioxane (5 mL) and stirred 48 h at 100° C. The OL (water/MTBE) was layer was dried and concentrated to give Int. 1AF82 3-Chloro-5-fluoro-N,N-dimethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide/(2-((4-(2-chloro-4-(dimethyl-carbamoyl)-6-fluorophenyl)-3,6-dihydropyridin-1(2H)-yl)methyl)-1-methyl-1H-pyrrolo[2,3-b]pyridin-4-yl)-boronic acid (0.19 g).

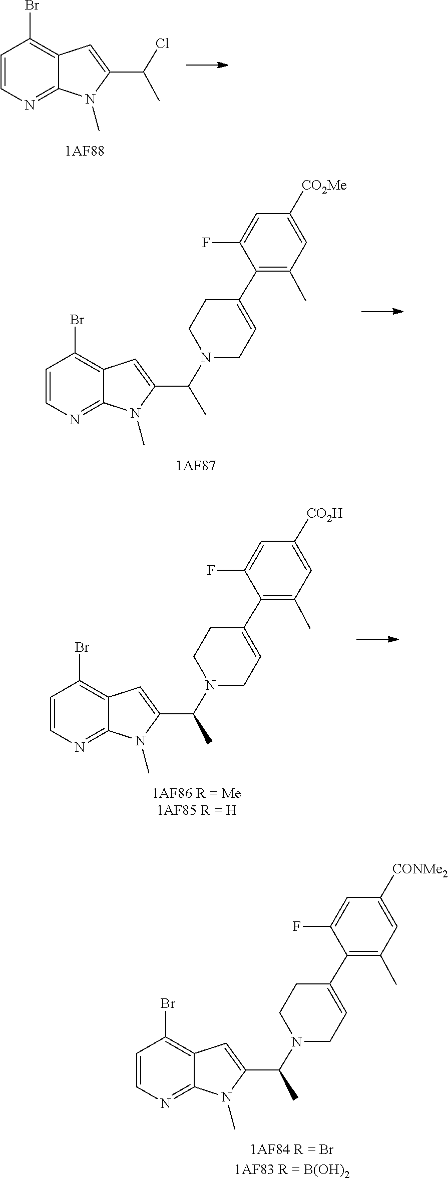

Int. 1AF21 (8 g) was stirred 0.5 h in toluene/SOCl₂ (4:1, 123 mL) at 0° C. to 80° C. and concentrated to give Int. 1AF88 (7.5 g). Ints. 1AF88/1AF90 (7.5 g, 7.8 g, HCl salt), Cs₂CO₃ (31 g), and KI (23 g) were stirred ON in ACN (100 mL) at 0° C. to RT. The mixture was filtered, concentrated, and purified by FC (hexane/EtOAc 41:9) to give Int. 1AF87 methyl 4-[1-[1-(4-bromo-1-methyl-pyrrolo[2,3-b]pyridin-2-yl)ethyl]-3,6-dihydro-2H-pyridin-4-yl]-3-fluoro-5-methyl-benzoate (3.9 g). Int. 1AF87 (0.2 g) was resolved by SFC using a Chiralpak AD-H 250×21 mm 5 μm column operated at 30° C. and an eluent of 70% CO₂ and 30% MeOH (70 g/min) and a back pressure of 100 bar to give Int. 1AF86 methyl(S)-4-(1-(1-(4-bromo-1-methyl-1H-pyrrolo[2,3-b]pyridin-2-yl)ethyl)-1,2,3,6-tetrahydropyridin-4-yl)-3-fluoro-5-methylbenzoate (70 mg, second peak). The absolute configuration was determined by VCD for Int. 1AF86. Int. 1AF86 (2.5 g) and LiOH (1.1 g) were stirred 1 h in THF/MeOH/water (2:1:1, 20 mL) at 0° C. to RT. The mixture was concentrated and triturated in aq. citric acid to precipitate Int. 1AF85 4-[1-[(1S)-1-(4-bromo-1-methyl-pyrrolo[2,3-b]pyridin-2-yl)ethyl]-3,6-dihydro-2H-pyridin-4-yl]-3-fluoro-5-methyl-benzoic acid (2.3 g). 4-[1-[(1S)-1-(4-bromo-1-methyl-pyrrolo[2,3-b]pyridin-2-yl)ethyl]-3,6-dihydro-2H-pyridin-4-yl]-3-fluoro-5-methyl-benzoic acid (1.8 g), HATU (2.2 g), and HNMe₂ (0.93 g, HCl salt) were stirred ON in DMF/DIPEA (6.1:1, 23.3 mL) at 0° C. to RT. The mixture was diluted with water to precipitate Int. 1AF84 4-[1-[(1S)-1-(4-bromo-1-methyl-pyrrolo[2,3-b]pyridin-2-yl)ethyl]-3,6-dihydro-2H-pyridin-4-yl]-3-fluoro-N,N,5-trimethyl-benzamide (1.5 g). Int. 1AF84′ (S)-4-(1-(1-(4-bromo-1-methyl-1H-pyrrolo[2,3-b]pyridin-2-yl)ethyl)-1,2,3,6-tetrahydropyridin-4-yl)-3-fluoro-5-methyl-N,N-bis(methyl-d₃) benzamide was prepared similarly using the HCl salt of HN(CD₃)₂. Int. 1AF84 (1.1 g), KOAc (0.86 g), B₂Pin₂ (1.1 g), and PdDPPFCl₂-DCM (0.18 g) stirred 1 h in dioxane (10 mL) at 110° C. under MW conditions. The mixture was filtered, concentrated, and triturated in pentane to give Int. 1AF83 [2—[(1S)-1-[4-[4-(dimethylcarbamoyl)-2-fluoro-6-methyl-phenyl]-3,6-dihydro-2H-pyridin-1-yl] ethyl]-1-methyl-pyrrolo[2,3-b]pyridin-4-yl]boronic acid (1.3 g).

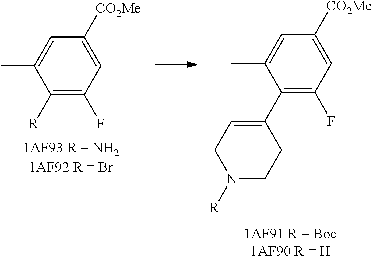

2-Fluoro-4-iodo-6-methyl-aniline (20 g) and PdDPPFCl₂-DCM were stirred ON in MeOH/Et₃N (23:1, 522 mL) under an atmosphere of CO (200 psi) at 80° C. The mixture was filtered. The OL (EtOAc/10% aq. EDTA) was dried and concentrated to afford Int. 1AF93 methyl 4-amino-3-fluoro-5-methyl-benzoate (14 g). Int. 1AF93 (25 g), tert-butyl nitrite (21.1 g), and CuBr₂ (152 g) were stirred ON in ACN (500 mL) at 0° C. to 80° C. The mixture was cooled to 0° C., diluted with sat. aq. NaHCO₃, and filtered. The OL (EtOAc/10% aq. NH₃) was concentrated and purified by FC (hexane/EtOAc 9:1) to give Int. 1AF92 methyl 4-bromo-3-fluoro-5-methyl-benzoate (21 g). Int. 1AF92 (1.0 g), tert-butyl 4-(4,4,5,5-tetramethyl-1,3,2-dioxaborolan-2-yl)-3,6-dihydropyridine-1(2H)-carboxylate (1.9 g), NaHCO₃ (1.29 g), and PdDPPFCl₂-DCM (0.33 g) were degassed in dioxane/water (5:1, 18 mL) and stirred ON at 110° C. The mixture was filtered. The OL (EtOAc/water) was dried, concentrated, and purified by FC (hexane/EtOAc 7:3) to give Int. 1AF91 tert-butyl 4-(2-fluoro-4-(methoxycarbonyl)-6-methyl-phenyl)-3,6-dihydro-pyridine-1(2H)-carboxylate (0.65 g). Int. 1AF91 (10 g) was stirred in 1.5 M HCl in dioxane (160 mL) at 0° C. to RT. The residue after concentration was triturated in Et₂O to give Int. 1AF90 methyl 3-fluoro-5-methyl-4-(1,2,3,6-tetrahydropyridin-4-yl)benzoate (13 g, HCl salt).

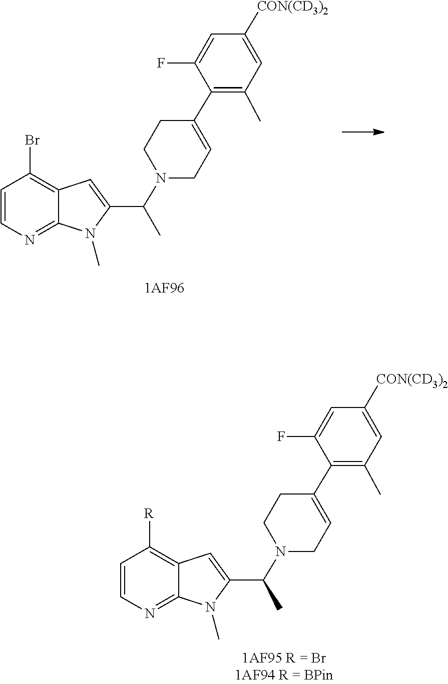

Ints. 1AF88/1AE12 (4.5 g, 5.5 g, HCl salt), Cs₂CO₃ (27 g), and KI (1.4 g) were stirred ON in ACN (50 mL) at 0° C. to RT. The mixture was diluted with water, filtered, and concentrated. The OL (EtOAc/water) was dried and concentrated. The residue was mixed with another batch prepared similarly on 1.5 g scale and purified by FC (pentane/EtOAc 3:7) to give Int. 1AF96 (4.8 g). Int. 1AF96 (5.0 g) was resolved by SFC using a Lux Cellulose.2 250×30 mm 5 μm column operated at 30° C. and an eluent of 70% CO₂ and 30% IPA (90 g/min) and a back pressure of 120 bar to give Int. 1AF95 (1.5 g, peak 2). Int. 1AF95 (0.5 g), B₂Pin₂(0.38 g), KOAc (0.29 g), and PdDPPFCl₂-DCM (81 mg) were degassed in dioxane (5 mL) and stirred 1 h at 120° C. under MW conditions. The mixture was concentrated and triturated in pentane/Et₂O to give Int. 1AF94 3-fluoro-5-methyl-4-[1-[(1S)-1-[1-methyl-4-(4,4,5,5-tetramethyl-1,3,2-dioxaborolan-2-yl) pyrrolo[2,3-b]-pyridin-2-yl]ethyl]-3,6-dihydro-2H-pyridin-4-yl]-N,N-bis(trideuteriomethyl)benzamide (0.6 g).

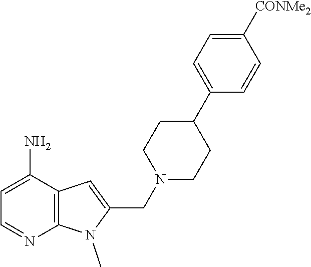

Int. 1AB1 (0.29 g), CuI (0.12 g), and NaN₃ (83 mg) were degassed in DMSO/DMEDA (14.4:1, 1.4 mL) and stirred at 100° C. The OL (sat. aq. NH₄Cl/EtOAc) was washed with water, brine, dried, concentrated, HPLC-purified, and triturated in MTBE to give Int. 1C₁₋₄-[1-[(4-Amino-1-methyl-pyrrolo[2,3-b]pyridin-2-yl)methyl]-4-piperidyl]-N,N-dimethyl-benzamide (0.22 g).

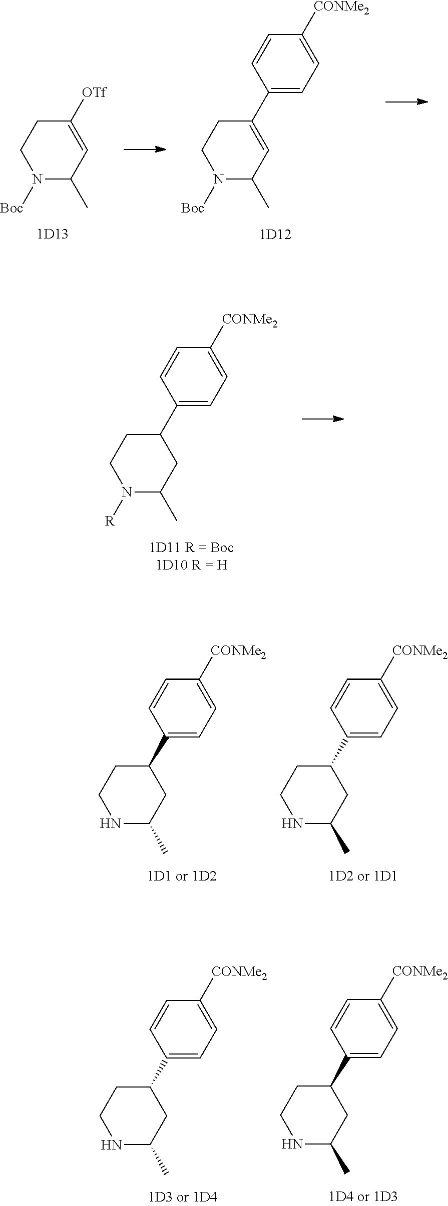

2.0M LDA in THF (28.1 mL) was added to a solution of tert-butyl 2-methyl-4-oxo-piperidine-1-carboxylate (10.0 g) in THF (200 mL)−78° C. The mixture was stirred for 0.3 h at −78° C. 1,1,1-Trifluoro-N-phenyl-N-((trifluoro-methyl) sulfonyl) methane-sulfonamide (20 g in THF (10 mL)) was added and stirring continued 3 h at −78° C. to RT. The OL (aq. NH₄Cl/EtOAc) was washed with water and brine, dried, and concentrated to give Int. 1D13 tert-butyl 6-methyl-4-(trifluoromethyl-sulfonyloxy)-3,6-dihydro-2H-pyridine-1-carboxylate (18.0 g). Int. 1D13 (10 g), Na₂CO₃(7.67 g), (4-(dimethyl-carbamoyl)phenyl) boronic acid (8.38 g), and PdDPPFCl₂-DCM (1.20 g) were degassed in toluene/water (5:1, 120 mL) and refluxed ON. The OL (water/EtOAc) was dried, concentrated, and purified by FC (hexane/EtOAc 7:3) to give Int. 1D12 tert-butyl 4-[4-(dimethyl-carbamoyl)phenyl]-6-methyl-3,6-dihydro-2H-pyridine-1-carboxylate (5.2 g). 5.0 g of this material was hydrogenated ON using 10% Pd/C (2.50 g) and H2 (50-60 psi) in EtOH (75 mL), filtered, concentrated, and purified by FC (hexane/EtOAc 35:65) to give Int. 1D11 tert-butyl 4-[4-(dimethylcarbamoyl)phenyl]-2-methyl-piperidine-1-carboxylate (4.0 g). 5.0 g of this material was stirred ON in DCM/TFA (3.6:1, 26 mL) at 0° C. to RT, concentrated, and triturated in Et₂O/pentane to give Int. 1D10 N,N-dimethyl-4-(2-methyl-4-piperidyl)benzamide (4.0 g, TFA salt). Int. 1D10 (10.8 g) was separated into the racemic cis and trans diastereomers by SFC using a Chiral Art Cellulose SC 250×30 5 μm column operated at 30° C. and an eluent of 60% CO₂ and 40% MeOH containing 0.5% HNEt₂ (90 g/min) and a back pressure of 120 bar to give the cis racemate (first two peaks; 3.0 g) and the trans racemate (last two peaks; 7.0 g). The trans racemate (7.0 g) was resolved by SFC on a Sepiatec instrument fitted with a Chiralpak IK 250×30 5 μm column operated at 30° C. and an eluent of 70% CO₂ and 30% MeOH containing 0.5% NH₃ at a (90 g/min) and a back pressure of 100 bar to give Int. 1D1 N,N-dimethyl-4-((2S,4R)-2-methylpiperidin-4-yl)benzamide (0.95 g, first peak) and Int. 1D2 N,N-dimethyl-4-((2R,4S)-2-methylpiperidin-4-yl)benzamide (1.2 g, second peak). The absolute configuration was determined by VCD for Int. 1D1 and 1D2. The cis racemate (3.0 g) was resolved by SFC using a Chiralpak IGK 250×30 5 μm column operated at 30° C. and an eluent MeOH containing 0.5% NH₃ (30 mL/min) and a back pressure of 100 bar to give Int. 1D3 N,N-dimethyl-4-((2R,4R)-2-methylpiperidin-4-yl)benzamide (0.70 g, first peak) and Int. 1D4 N,N-dimethyl-4-((2S,4S)-2-methyl-piperidin-4-yl)benzamide (0.90 g, second peak). The absolute configuration was determined by VCD for Int. 1D3 and 1D4.

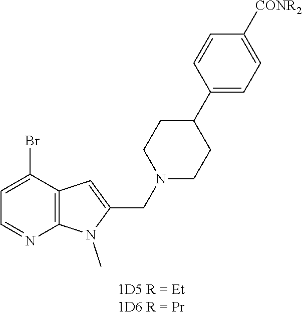

Int. 1CA4 (20 mg), HNEt₂ (24 uL), and HATU (27 mg) were stirred 0.25 h in DMF (1 mL). The OL (sat. aq. NaHCO/EtOAc) was washed with brine, dried, and concentrated to give Int. 1D5 4-(1-((4-Bromo-1-methyl-1H-pyrrolo[2,3-b]pyridin-2-yl)methyl) piperidin-4-yl)-N,N-diethylbenzamide (30 mg). Int. 1D6 4-(1-((4-bromo-1-methyl-1H-pyrrolo[2,3-b]pyridin-2-yl)methyl) piperidin-4-yl)-N,N-dipropyl-benzamide (37 mg, was prepared similarly from HNPr₂.

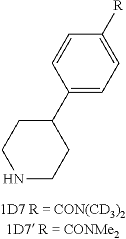

1-(1,1-Dimethylethyl) 4-(4-carboxyphenyl)-1-piperidinecarboxylate (5.1 g), HATU (13 g), and HN(CD3)₂ (2.5 g, HCl salt) were stirred ON in DMF/DIPEA (1.8:1, 47 mL). Water was added to precipitate solid that was dissolved in DCM/MeOH (9:1), dried, concentrated, and purified by FC (hexane/EtOAc 2:3) to give tert-butyl 4-[4-[bis-(trideuteriomethyl)carbamoyl]phenyl]piperidine-1-carboxylate (4.8 g). 1.1 g of this material was stirred ON in DCM/TFA (3.6:1, 26 mL) at 0° C. to RT, concentrated, and triturated in Et₂O to give Int. 1D7 N,N-Bis (methyl-d₃)-4-(112-piperidin-4-yl)-benzamide (0.81 g, TFA salt). Int. 1D7′ N,N-dimethyl-4-(4-piperidyl)benzamide (9 g, HCl salt) was prepared similarly from 1-(1,1-dimethylethyl) 4-(4-carboxyphenyl)-1-piperidine-carboxylate (17 g) and HN(CH₃)₂ (13.6 g, HCl salt).

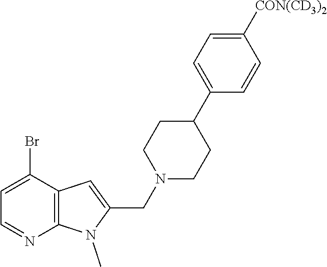

Int. 1AC4 (4.0 g), EDC (4.7 g, HCl salt), HOBt (2.5 g), and HN(CD₃)₂ (1.4 g, HCl salt) were stirred ON in DMF/DIPEA (8.2:1, 89 mL). The residue after concentration was purified by FC (EtOAc/hexane) to give Int. 1D8 4-(1-((4-bromo-1-methyl-1H-pyrrolo[2,3-b]pyridin-2-yl)methyl) piperidin-4-yl)-N,N-bis(methyl-d₃) benzamide (2.7 g).

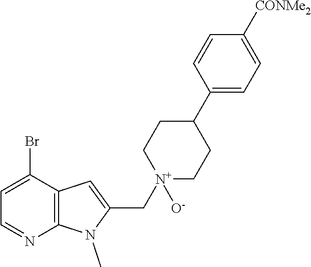

Int. 1AB1 (1 g) mCPBA (0.59 g) were stirred 2 h in DCM (10 mL) at 0° C. The OL (sat. aq. NaHCO₃/DCM) was dried and concentrated. The residue was stirred 1 h in TFA, concentrated, and purified by FC (DCM/MeOH 1:0 to 4:1) to give Int. 1D9 4-[1-[(4-bromo-1-methyl-pyrrolo[2,3-b]pyridin-2-yl)methyl]-1-oxido-piperidin-1-ium-4-yl]-N,N-dimethyl-benzamide (0.77 g).

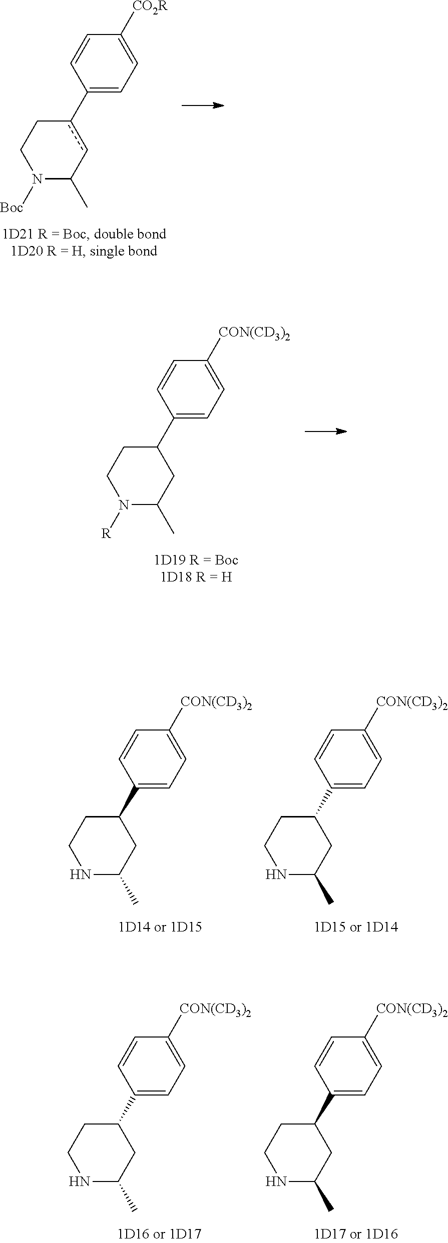

Int. 1D21 tert-butyl 4-(4-(methoxycarbonyl)phenyl)-6-methyl-3,6-dihydropyridine-1(2H)-carboxylate (5.20 g) was prepared similarly to Int. 1D12 from Int. 1D13 (10.0 g) and (4-(methoxycarbonyl)phenyl) boronic acid (7.82 g). Int. 1D20 tert-butyl 4-(4-(methoxycarbonyl)phenyl)-2-methylpiperidine-1-carboxylate (3.0 g) was prepared similarly to Int. 1D11 from Int. 1D21 (3.5 g). Int. 1D20 (3.0 g) and LiOH—H₂O (1.50 g) were stirred 4 h in THF/MeOH/water (1.3:1:1, 50 mL) at 0° C. to RT and concentrated. The OL (aq. citric acid/EtOAc) was dried and concentrated to give 4-(1-tert-butoxy-carbonyl-2-methyl-4-piperidyl)benzoic acid (1.50 g) 6.0 g of this material, HN(CD)₃)₂ (2.47 g, HCl salt), HOBt (1.90 g), and EDC-HCl (2.70 g) were stirred ON in DMF/DIPEA (1.9:1, 46 mL) at 0° C. to RT. The OL (water/EtOAc) was dried and concentrated to give Int. 1D19 tert-butyl 4-(4-(bis(methyl-ds)carbamoyl)-phenyl)-2-methylpiperidine-1-carboxylate (4.50 g). 3.5 g of this material was stirred 4 h in DCM/TFA (2.5:1, 70 mL) at 0° C. to RT, concentrated, and triturated in Et₂O/pentane to give Int. 1D18 4-(2-methyl-4-piperidyl)-N,N-bis(trideuteriomethyl)benzamide (3.50 g, TFA salt). Int. 1D18 was separated into Ints. 1D14 N,N-bis(methyl-d₃)-4-((2S,4R)-2-methylpiperidin-4-yl)benzamide, Int. 1D15 N,N-bis-(methyl-d₃)-4-((2R,4S)-2-methylpiperidin-4-yl)benzamide, Int. 1D16 N,N-bis(methyl-d₃)-4-((2R,4R)-2-methylpiperidin-4-yl)benzamide, and Int. 1D17 N,N-bis(methyl-3)-4-((2S,4S)-2-methyl-piperidin-4-yl)benzamide as described for Ints. 1D1-1D4. The absolute configurations of these compounds were not determined. The absolute configurations of these compounds were determined by comparison to RT on chiral chromatography vs. Int. 1D1-1D4.

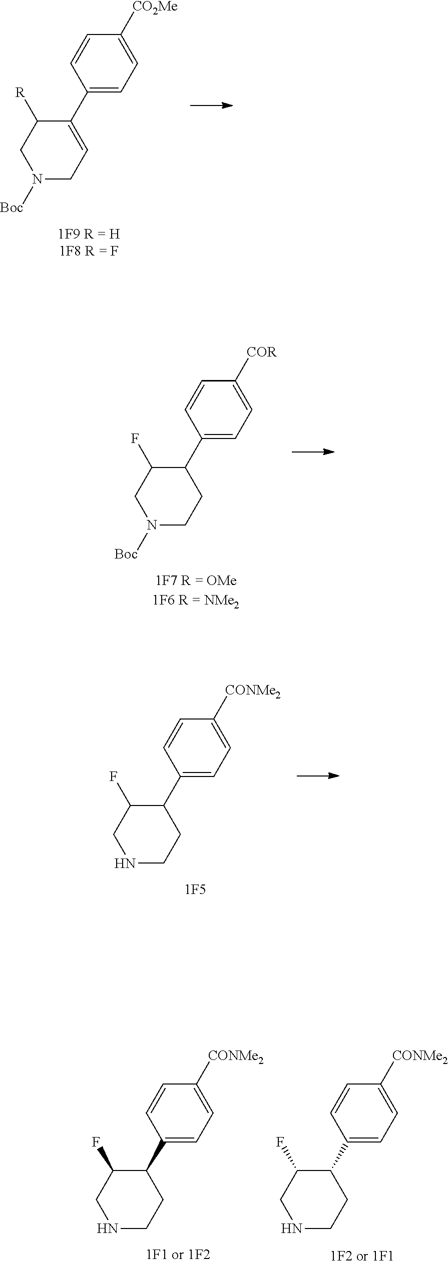

Methyl 4-bromobenzoate (25 g), PdDPPFCl₂-DCM (4.7 g), NaHCO₃ (49 g), and tert-butyl 4-(4,4,5,5-tetramethyl-1,3,2-dioxaborolan-2-yl)-3,6-dihydropyridine-1(2H)-carboxylate (39 g) were degassed in 1,4-dioxane/water (5:1, 0.6 L) and stirred ON at 110° C. The OL (water/EtOAc) was washed with brine, dried, concentrated, and purified by FC (pentane/EtOAc 99:1) to give Int. 1F9 tert-butyl 4-(4-(methoxycarbonyl)-phenyl)-3,6-dihydro-pyridine-1(2H)-carboxylate (18 g). Int. 1F9 (38 g) and SelectFluor (7.2 g) were stirred 1 h in ACN/water (3:1, 0.64 L). SelectFluor (2.1 g) was added and stirring continued 1 h. The OL (EtOAc/sat. aq. NaHCO₃) was dried, concentrated, and purified by FC (hexane/EtOAc 85:15) to give Int. 1F8 tert-butyl-3-fluoro-4-(4-methoxy-carbonylphenyl)-3,6-dihydro-2H-pyridine-1-carboxylate (6.0 g). Int. 1F8 (4.8 g) was hydrogenated ON using 10% Pd/C (0.48 g) in EtOAc (200 mL), filtered, concentrated, and purified by FC (hexane/EtOAc 9:1) to give Int. 1F7 tert-butyl-3-fluoro-4-(4-methoxycarbonylphenyl) piperidine-1-carboxylate (3.2 g). Int. 1F7 (3.5 g) and LiOH—H₂O (1.4 g) were stirred ON in MeOH/THF/H₂O (3:2:1; 25 mL) at 0° C. to RT and concentrated. The OL (10% MeOH in DCM/aq. citric acid) was dried and concentrated to give cis-4-(1-(tert-butoxycarbonyl)-3-fluoro-piperidin-4-yl)benzoic acid (2.1 g). 2.5 of this compound, HATU (4.4 g), and HN(CH₃)₂ (1.9 g, HCl salt) were stirred ON in DMF/DIPEA (2.4:1, 35 mL) at 0° C. to RT. The OL (water/5% MeOH in DCM) was dried, concentrated, and purified by FC (DCM/MeOH 95:5) to give Int. 1F6 cis-tert-butyl-4-[4-(dimethylcarbamoyl)-phenyl]-3-fluoro-piperidine-1-carboxylate (2.4 g). Int. 1F6 (1.5 g) was stirred ON in DCM/TFA (3,8:1, 13 mL) at 0° C. to RT and concentrated. The OL (aq. NaHCO₃/Et₂O and 10% MeOH in DCM) was dried and concentrated to give Int. 1F5 N,N-dimethyl-4-[cis-3-fluoro-4-piperidyl]-benzamide (1.19 g). This material was resolved by SFC using a Lux Cellulose-4 250×30 5 μm column operated at 30° C. and an eluent of 70% CO₂ and 30% 0.5% iso-propyl amine in IPA (100 g/min) and a back pressure of 100 bar to give Int. 1F1 (0.47 g) and Int. 1F2 (0.50 g) 4-((3S,4R)-3-Fluoropiperidin-4-yl)-N,N-dimethylbenzamide and 4-((3R,4S)-3-fluoropiperidin-4-yl)-N,N-dimethyl-benzamide. The absolute configurations of these compounds were not determined.

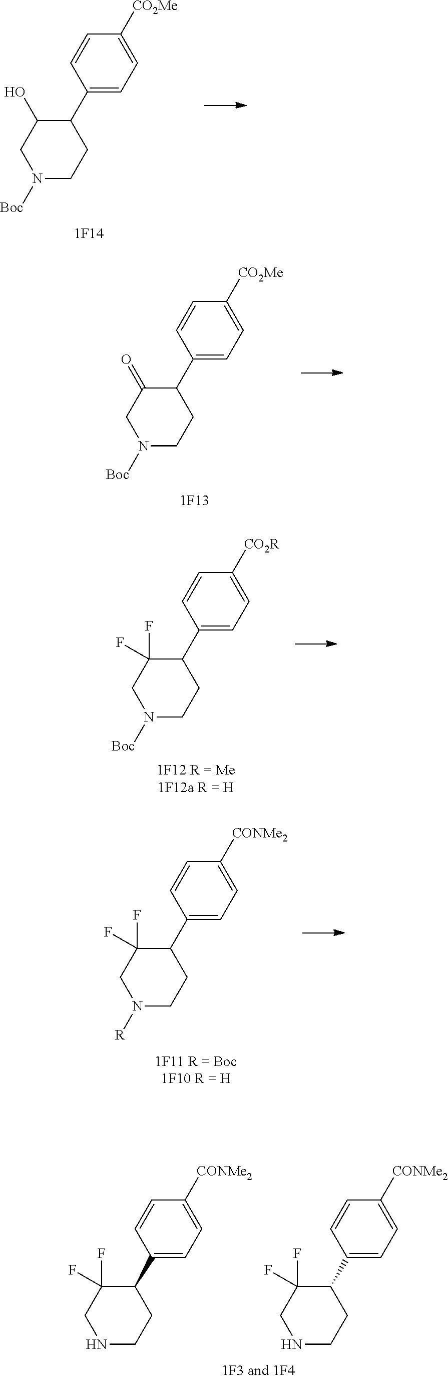

Int. 1F9 (10 g) was stirred ON in THF/2M BH₃-DMS in THF (11.6:1, 217 mL) at 0° C. to RT. 2M aq. NaOH (38 mL) and 30% aq. H₂O₂ (5.9 mL) were added and stirring was continued at 0° C. to RT over 1 h. The OL (sat. aq. NaS₂O₃/EtOAc) was washed with water and brine, dried, concentrated, and purified by FC (hexane/EtOAc 7:3) to give Int. 1F14 tert-butyl 3-hydroxy-4-(4-(methoxy-carbonyl)phenyl) piperidine-1-carboxylate (7.4 g). Int. 1F14 (8.0 g) and DMP (5.1 g) were stirred ON in DCM (200 mL) at 0° C. to RT. The OL (water/DCM) was dried, concentrated, and purified by FC (hexane/EtOAc 9:1) to give Int. 1F13 tert-butyl 4-(4-methoxy-carbonyl-phenyl)-3-oxo-piperidine-1-carboxylate (5.2 g). Int. 1F13 (5.0 g) was stirred ON in DCM/50% Deoxo-Fluor in THF (12.2:1, 108 mL) at −78° C. to RT ON. The OL (aq. NaHCO₃/DCM) was washed with sat. aq. citric acid, dried, concentrated, and purified by FC (hexane) to give Int. 1F12 tert-butyl 3,3-difluoro-4-(4-methoxycarbonyl-phenyl) piperidine-1-carboxylate (2.5 g). This material and LiOH—H₂O (1.5 g) were stirred in MeOH/THF/H₂O (3:3:1; 60 mL) at 0° C. to RT over 4 h, concentrated, and triturated in dilute aq. citric acid to give Int. 1F12a 4-(1-tert-butoxycarbonyl-3,3-difluoro-4-piperidyl)benzoic acid (2.3 g). Int. 1F12a (4.8 g), HATU (6.4 g), and HN(CH₃)₂ (2.3 g, HCl salt) were stirred ON in DMF/DIPEA (13.2:1, 108 mL) at 0° C. to RT. The OL (water/EtOAc) was dried, concentrated, and purified by FC (hexane/EtOAc 1:4) to give Int. 1F11 tert-butyl 4-(4-(dimethylcarbamoyl)-phenyl)-3,3-difluoro-piperidine-1-carboxylate (4.5 g). Int. 1F11 (4.2 g) was stirred ON in DCM/TFA (27:1, 83 mL) at 0° C. to RT and concentrated. The OL (5% MeOH in DCM/sat. aq. NaHCO₃) was dried and concentrated to give Int. 1F10 4-(3,3-difluoro-4-piperidyl)-N,N-dimethyl-benzamide (3 g). This material was resolved by SFC using a Chiralpak IG 250×30 5 μm column operated at 30° C. and an eluent of 60% CO₂ and 40% MeOH (80 g/min) and a back pressure of 60 bar to give Int. 1F3 (1.1 g) and Int. 1F4 (1.0 g) (R)-4-(3,3-Difluoropiperidin-4-yl)-N,N-dimethylbenzamide and(S)-4-(3,3-difluoropiperidin-4-yl)-N,N-dimethylbenzamide. The absolute configurations of these compounds were not determined.

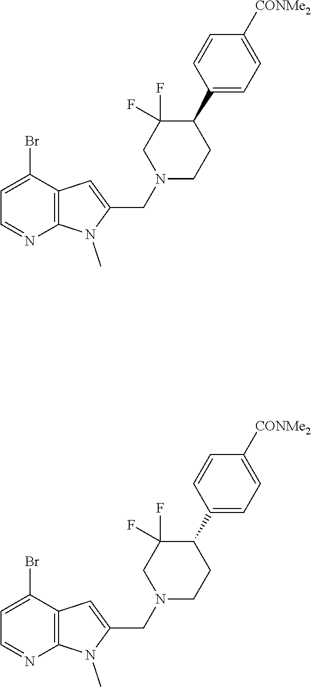

Ints. 1J1/1F3 (45 mg/60 mg), Cs₂CO₃(169 mg), NaI (26 mg) were stirred in DMF (1 mL) for 0.5 h and HPLC-purified to give Int. 1F15(48 mg). Int. 1F16 was prepared similarly from Int. 1F4. Ints. 1F15 and 1F16 (R)-4-(1-((4-bromo-1-methyl-1H-pyrrolo[2,3-b]pyridin-2-yl)methyl)-3,3-difluoropiperidin-4-yl)-N,N-dimethylbenzamide and(S)-4-(1-((4-bromo-1-methyl-1H-pyrrolo[2,3-b]pyridin-2-yl)methyl)-3,3-difluoropiperidin-4-yl)-N,N-dimethylbenzamide. The absolute configurations were not determined for these compounds.

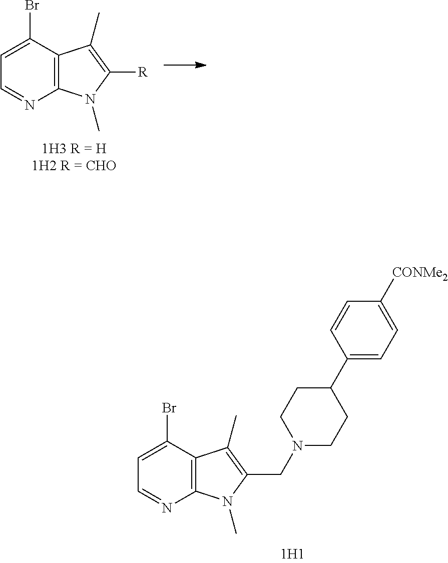

4-Bromo-3-methyl-1H-pyrrolo[2,3-b]pyridine (0.51 g) and NaH (0.12 g) were stirred 1 h in DMF (5 mL) at 0° C. before MeI (0.3 mL) was added and stirring continued for 0.3 h. The OL (water/EtOAc/Et₂O) was washed with brine, dried, concentrated to give Int. 1H3 4-bromo-1,3-dimethyl-pyrrolo[2,3-b]pyridine (0.53 g). This material was stirred 2 h in THF/1.0 M LDA in THF (2.9:1, 13.5 mL) −78° C. before DMF (1 mL) was added and stirring continued 2 h at −78° C. to RT. The OL (sat. aq. NH₄Cl/EtOAc) was washed with brine, dried, concentrated, and purified by FC (heptane/EtOAc 1:0 to 1:1) to give Int. 1H2 4-bromo-1,3-dimethyl-pyrrolo[2,3-b]pyridine-2-carbaldehyde (0.17 g). Int. 1H2 (30 mg) and N,N-dimethyl-4-(piperidin-4-yl)benzamide (42 mg) were stirred 0.5 h in DCE (2 mL). STAB (57 mg) was added and stirring continued ON. The OL (sat. aq. NaHCO/DCM.) was washed with brine, dried, concentrated, and purified by FC (DCM/MeOH 1:0 to 9:1) to give Int. 1H1 4-[1-[(4-bromo-1,3-dimethyl-pyrrolo[2,3-b]pyridin-2-yl)methyl]-4-piperidyl]-N,N-dimethyl-benzamide (30 mg).

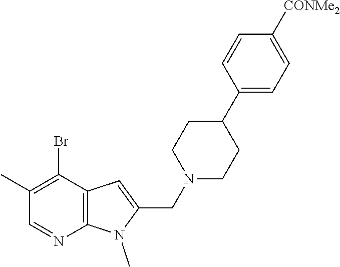

Int. 111 4-(1-((4-bromo-1,5-dimethyl-1H-pyrrolo[2,3-b]pyridin-2-yl)methyl) piperidin-4-yl)-N,N-dimethylbenzamide (0.16 g) as prepared similarly to Int. 1H1 from 4-bromo-5-methyl-1H-pyrrolo[2,3-b]pyridine (0.51 g) and N,N-dimethyl-4-(piperidin-4-yl)benzamide (0.18 g).

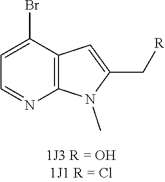

Int. 1AB8 (30 g) and NaBH₄ (19 g) were stirred 2 h in MeOH (300 mL) at 0° C. to RT. The OL (aq. Na₂S₂O₃/EtOAc) was dried, concentrated, and purified by FC (pentane/EtOAc 1:1 to 2:3) to give Int. 1J3 (4-bromo-1-methyl-pyrrolo[2,3-b]pyridin-2-yl) methanol. Int. 1J3 (2.5 g) was stirred ON in DCM/Et₃N/MsCl (21.4:5.2:1, 39 mL) at 0° C. to RT and filtered. The OL layer (EtOAc/water) was washed with brine, dried, and concentrated to give Int. 1J1 4-bromo-2-(chloromethyl)-1-methyl-1H-pyrrolo[2,3-b]pyridine (2.0 g).

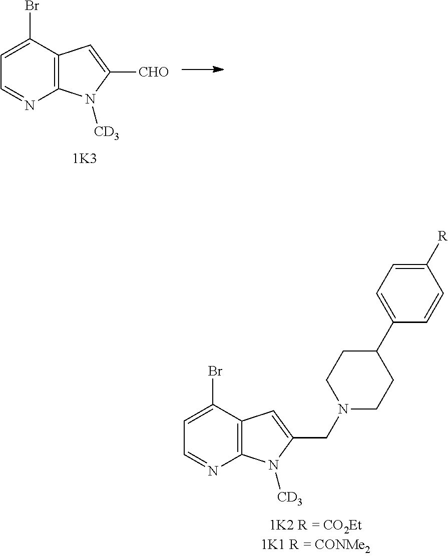

4-Bromo-1-(trideuteriomethyl) pyrrolo[2,3-b]pyridine (0.40 g) was stirred 2 h in THF/2M LDA in THF (7.1:1, 11.4 mL) at −78° C. DMF (0.7 mL) was added and stirring continued 2 h at −78° C. to RT and diluted with water to precipitate Int. 1K3 4-bromo-1-(trideuterio-methyl) pyrrolo[2,3-b]pyridine-2-carbaldehyde (0.35 g). Int. 1K3 (0.17 g) and methyl 4-(piperidin-4-yl)benzoate (0.16 g) were stirred 3 h in DCE/DIPEA (28.3:1, 15.5 mL). STAB (0.26 g) was added and stirring continued ON. The OL (water/DCM) was washed brine, dried, concentrated, and purified by FC (pentane/EtOAc 4:1) to give Int. 1K2 methyl 4-[1-[4-bromo-1-(trideuteriomethyl) pyrrolo-[2,3-b]pyridin-2-yl]methyl]-4-piperidyl]-benzoate (0.14 g). Int. 1K2 (50 mg) was stirred 0.25 h in THF/MeOH/2M aq. NaOH (7.1:3.6:1, 3.3 mL). pH was adjusted to 5 with 1M aq. HCl. The mixture was concentrated. The residue and HATU (85 mg) were dissolved in DMF/DIPEA (25.6:1, 2.1 mL) before HN(CH₃)₂ (14 mg; HCl salt) was added and stirring continued 72 h. The OL (water/DCM) was dried, concentrated, and purified by FC (DCM/MeOH 1: to 9:1) to give Int. 1K1 4-[1-[4-bromo-1-(trideuteriomethyl) pyrrolo[2,3-b]pyridin-2-yl]-methyl]-4-piperidyl]-N,N-dimethyl-benzamide (39 mg).

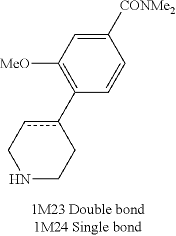

4-Bromo-3-methoxybenzoic acid (3.0 g), HN(CH₃)₂ (1.6 g, HCl salt), and HATU (5.92 g) were stirred ON in DCM/DIPEA (7.4:1, 57 mL), concentrated, and HPLC-purified to give 4-bromo-3-methoxy-N,N-dimethyl-benzamide (2.5 g). 1.0 g of this material, tert-butyl 4-(4,4,5,5-tetramethyl-1,3,2-dioxaborolan-2-yl)-3,6-dihydropyridine-1(2H)-carboxylate (1.44 g), K₂CO₃(2.14 g), and PdDPPFCl₂-DCM (0.16 g) were degassed in dioxane/water (4:1, 10 mL) and stirred at 100° C. ON. The OL (water/EtOAc) was washed with brine, dried, concentrated, and purified by FC (hexane to EtOAc) to give tert-butyl 4-(4-(dimethylcarbamoyl)-2-methoxy-phenyl)-3,6-dihydropyridine-1(2H)-carboxylate (1.10 g). 0.6 g of this material was hydrogenated ON using 10% Pd/C (0.2 g) in MeOH (10 mL), filtered, concentrated, stirred in 4M HCl in dioxane (10 mL) ON, concentrated, and HPLC-purified to give Int. 1M24 3-methoxy-N,N-dimethyl-4-(piperidin-4-yl)benzamide (0.23 g). tert-Butyl 4-(4-(dimethylcarbamoyl)-2-methoxyphenyl)-3,6-dihydropyridine-1(2H)-carboxylate (0.50 g) was stirred ON in 4M HCl in dioxane (10 mL), concentrated, and HPLC-purified to give Int. 1M23 3-methoxy-N,N-dimethyl-4-(1,2,3,6-tetrahydropyridin-4-yl)benzamide (0.17 g).

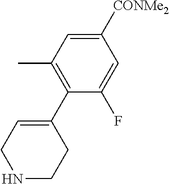

NaNO₂ (6.1 g) and 4-amino-3-fluoro-5-methyl-benzoic acid (15.0 g) were stirred 0.3 h in 48% aq. HBr (150 mL) at 0° C. CuBr (13.0 g) was added and stirring continued 4 h. The OL (aq. NaHCO₃/MTBE) was washed with water and concentrated to give 4-bromo-3-fluoro-5-methyl-benzoic acid (8.50 g). 4-Bromo-3-fluoro-5-methyl-benzoic acid (15.0 g) was stirred 5 h in DCM/SOCl₂(83:1, 150 mL), and concentrated. The residue and HNMe₂ (3.1 g, HCl salt) were stirred 5 h DCM/Et₃N (13.6:1, 160 mL) The OL (water/DCM) was concentrated. The residue, K₂CO₃(7.3 g), PdPDPPFCl₂-DCM (0.15 g), and tert-butyl 4-(4,4,5,5-tetramethyl-1,3,2-dioxaborolan-2-yl)-3,6-dihydro-pyridine-1(2H)-carboxylate (6.0 g) were degassed dioxane (150 mL) and stirred 48 h at 90° C. The OL (water/dioxane) was concentrated, stirred 0.5 h in THF/4M HCl in dioxane (1:1, 100 mL), concentrated, and HPLC-purified to give Int. 1M25 3-fluoro-N,N,5-trimethyl-4-(1,2,3,6-tetrahydropyridin-4-yl)benzamide (0.22 g).

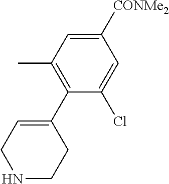

Int. 1M26 3-chloro-N,N,5-trimethyl-4-(1,2,3,6-tetrahydropyridin-4-yl)benzamide (0.11 g) was prepared similarly to Int. 1M25 from 4-bromo-3-chloro-5-methyl-benzoic acid (15 g).

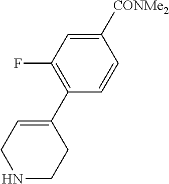

4-Bromo-3-fluoro-N,N-dimethyl-benzamide (1.0 g), tert-butyl 4-(4,4,5,5-tetramethyl-1,3,2-dioxa-borolan-2-yl)-3,6-dihydro-2H-pyridine-1-carboxylate (1.41 g), Na₂CO₃(1.61 g), and PdDPPFCl₂-DCM (0.16 g) were degassed in dioxane/water (4:1, 10 mL) and stirred ON at 100° C. The OL (water/EtOAc) was washed with brine, dried, concentrated, and purified by FC (hexane to EtOAc) to give tert-butyl 4-[4-(dimethylcarbamo-yl)-2-fluoro-phenyl]-3,6-dihydro-2H-pyridine-1-carboxylate (1.20 g). 0.6 g of this material was stirred ON in 4M HCl in dioxane (10 mL). The mixture was concentrated and HPLC-purified to give Int. 1M29 3-fluoro-N,N-dimethyl-4-(1,2,3,6-tetrahydropyridin-4-yl)-benzamide (0.22 g).

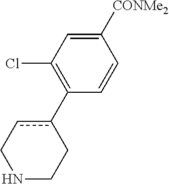

4-Bromo-3-chloro-N,N-dimethyl-benzamide (5.0 g), tert-butyl 4-(4,4,5,5-tetramethyl-1,3,2-dioxaborolan-2-yl)-3,6-dihydropyridine-1(2H)-carboxylate (0.71 g), Na₂CO₃(8.1 g), and PdDPPFCl₂-DCM (0.78 g) were degassed in dioxane/water (4:1, 10 mL) and stirred at 100° C. ON. The OL (water/EtOAc) was dried, concentrated, and purified by FC (hexane to EtOAc) to give tert-butyl 4-[2-chloro-4-(dimethylcarbamoyl)phenyl]-3,6-dihydro-2H-pyridine-1-carboxylate (1.10 g). This material was hydrogenated ON using 10% Pt/C (1.1 g) in MeOH (10 mL), filtered, and concentrated to give tert-butyl 4-[2-chloro-4-(dimethylcarbamoyl)phenyl]-piperidine-1-carboxylate (0.60 g). This material was stirred ON in 4M HCl in dioxane (10 mL), concentrated, and HPLC-purified to give Int. 1M44 3-chloro-N,N-dimethyl-4-(piperidin-4-yl)benzamide (0.15 g). tert-Butyl 4-[2-chloro-4-(dimethyl-carbamoyl)phenyl]-3,6-dihydro-2H-pyridine-1-carboxylate (0.60 g) was stirred ON in 4M HCl in dioxane (10 mL), concentrated, and HPLC-purified to give Int. 1M43 3-chloro-N,N-dimethyl-4-(1,2,3,6-tetrahydropyridin-4-yl)benzamide (0.22 g).

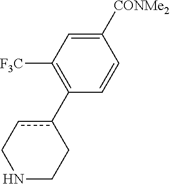

4-Bromo-3-(trifluoromethyl)benzoic acid (3.0 g), HN(CH₃)₂ (1.36 g, HCl salt) and HATU (5.1 g) stirred ON in DCM/DIPEA (8.6:1, 56 mL), concentrated, and HPLC-purified to give 4-bromo-N,N-dimethyl-3-(trifluoro-methyl)benzamide (2.40 g). 1.0 g of this material, tert-butyl 4-(4,4,5,5-tetramethyl-1,3,2-dioxaborolan-2-yl)-3,6-dihydro-pyridine-1(2H)-carboxylate (1.25 g), K₂CO₃(1.87 g), and PdDPPFCl₂-DCM (0.14 g) were degassed in dioxane/water (4:1, 10 mL) and stirred ON at 100° C. The OL (water/EtOAc) was washed with brine, dried, concentrated, and purified by FC (hexane to EtOAc) to obtain tert-butyl 4-[4-(dimethyl-carbamoyl)-2-(trifluoromethyl)phenyl]-3,6-dihydro-2H-pyridine-1-carboxylate (1.04 g). 0.50 g of this material was stirred ON in 4M HCl in dioxane (10 mL), filtered, and HPLC-purified to give Int. 1M45 N,N-dimethyl-4-(1,2,3,6-tetrahydropyridin-4-yl)-3-(trifluoro-methyl)benzamide (0.14 g). tert-Butyl 4-[4-(dimethyl-carbamoyl)-2-(trifluoromethyl)phenyl]-3,6-dihydro-2H-pyridine-1-carboxylate (0.50 g) was hydrogenated ON using 10% Pd/C (0.5 g) in MeOH (10 mL). The mixture was filtered. The residue after concentration was stirred ON in 4M HCl in dioxane (10 mL), concentrated, and HPLC-purified to give Int. 1M46 N,N-dimethyl-4-(piperidin-4-yl)-3-(trifluoromethyl)benzamide (0.23 g).

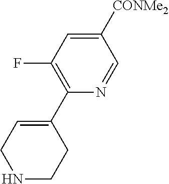

6-Chloro-5-fluoro-pyridine-3-carboxylic acid (3.0 g), HN(CH₃)₂ (1.53 g, HCl salt), and HATU (7.15 g) were stirred ON in DCM/DIPEA (5.6:1, 59 mL), concentrated, and purified by FC (hexane to EtOAc) to give 6-chloro-5-fluoro-N,N-dimethyl-pyridine-3-carboxamide (3.1 g). This material, tert-butyl 4-(4,4,5,5-tetramethyl-1,3,2-dioxa-borolan-2-yl)-3,6-dihydropyridine-1(2H)-carboxylate (5.20 g), K3CO₃ (8.50 g), and PdDPPFCl₂-DCM (0.62 g) were degassed in dioxane/water (4:1, 50 mL) and stirred ON at 100° C. The OL (EtOAc/water) was washed with brine, dried, concentrated, and purified by FC (hexane to EtOAc) to give tert-Butyl 4-[5-(dimethylcarbamoyl)-3-fluoro-2-pyridyl]-3,6-dihydro-2H-pyridine-1-carboxylate (3.10 g). 0.13 g of this material was stirred 1.5 h in DCM/4M HCl in dioxane (3.6:1, 6.4 mL) and concentrated. The OL (DCM/aq. NaHCO₃) was dried, concentrated, and HPLC-purified to give Int. 1M47 5-fluoro-N,N-dimethyl-6-(1,2,3,6-tetrahydropyridin-4-yl)pyridine-3-carboxamide (20 mg).

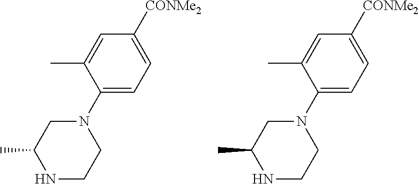

tert-Butyl(S)-2-methylpiperazine-1-carboxylate (1.0 g), ethyl 4-bromo-3-methyl-benzoate (1.46 g), Cs₂CO₃(4.88 g), Pd₂dba₃ (0.23 g), and XPhos (0.24 g) were degassed in dioxane (20 mL) and stirred at 110° C. ON. The OL (EtOAc/water) was concentrated and purified by FC (hexane to EtOAc) to give tert-butyl (2R)-4-(4-ethoxy-carbonyl-2-methyl-phenyl)-2-methyl-piperazine-1-carboxylate (0.85 g). 0.70 g of this material and LiOH—H₂O (0.22 g) were stirred 6 h in THF/water/MeOH (10 mL), concentrated, and triturated in dilute aq. citric acid to give 4-[(3R)-4-tert-butoxy-carbonyl-3-methyl-piperazin-1-yl]-3-methyl-benzoic acid (0.45 g). 4-[(3R)-4-tert-butoxycarbonyl-3-methyl-piperazin-1-yl]-3-methyl-benzoic acid (0.70 g), HNMe₂ (0.26 g, HCl salt), HOBt (0.42 g), and EDC (0.60 g, HCl salt) were stirred ON in DCM/DIPEA (10:1, 11 mL) at 0° C. to RT. The OL (water/DCM) was washed with brine, dried, concentrated, and purified by FC (hexane/EtOAc 3:2) to give tert-butyl (2R)-4-[4-(dimethylcarbamoyl)-2-methyl-phenyl]-2-methyl-piperazine-1-carboxylate (0.45 g). This material was stirred 6 h in DCM/TFA (6:1, 7 mL) at 0° C. to RT, concentrated, and triturated in Et₂O to give Int. 1M49 (R)—N,N,3-trimethyl-4-(3-methylpiperazin-1-yl)-benzamide (0.20 g). Int. 1M59 (S)—N,N,3-trimethyl-4-(3-methylpiperazin-1-yl)-benzamide was prepared similarly from tert-butyl (R)-2-methylpiperazine-1-carboxylate.

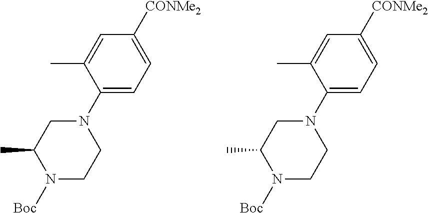

tert-Butyl(S)-2-methylpiperazine-1-carboxylate (2.5 g), NaOtBu (2.4 g), Pd₂dba₃ (0.57 g), Xphos (0.59 g), and 4-bromo-N,N-dimethylbenzamide (3.1 g) were degassed in dioxane (30 mL) and stirred ON at 100° C. The OL (Et₂O/water) was washed with brine, dried, concentrated, and purified by FC (hexane/EtOAc 1:4) to give Int. 1M50 tert-butyl(S)-4-(4-(dimethylcarbamoyl)phenyl)-2-methyl-piperazine-1-carboxylate and tert-butyl (2.4 g). Int. 1M52 (R)-4-(4-(dimethylcarbamoyl)phenyl)-2-methyl-piperazine-1-carboxylate (0.46 g) was prepared similarly from tert-butyl (R)-2-methylpiperazine-1-carboxylate (0.50 g).

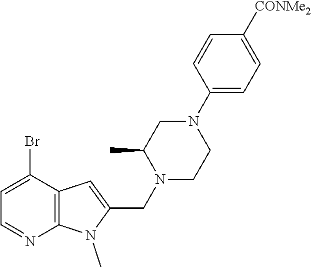

Int. 1M50 (46 mg) was stirred 1 h in DCM/TFA (1:1, 1 mL) and concentrated. The residue and 4 Å MS were stirred ON in DCM/DIPEA (19.5:1, 1.6 mL). Int. 1J1 (23 mg) was added and stirring was continued ON. The OL (DCM/sat. aq. NaHCO₃) was dried, concentrated, and purified by FC (DCM/MeOH 1:0 to 95:5) to give Int. 1M51 (S)-4-(4-((4-bromo-1-methyl-1H-pyrrolo[2,3-b]pyridin-2-yl)methyl)-3-methylpiperazin-1-yl)-N,N-dimethylbenzamide (40 mg).

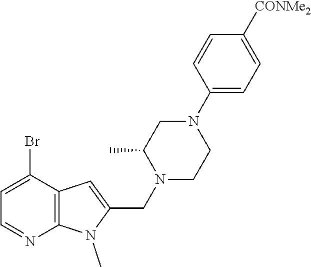

Int. 1M52 (46 mg) was stirred 1 h in DCM/TFA (1:1, 1 mL) and concentrated. The residue, Int. 1AB8 (21 mg), and 4 Å MS were stirred 2 h in DCM/DIPEA (19.5:1, 1.6 mL). STAB (56 mg) was added and stirring continued ON. The OL (water/DCM) was dried, concentrated, and purified by FC (DCM/MeOH 1:0 to 95:5) to give Int. 1M53 (R)-4-(4-((4-bromo-1-methyl-1H-pyrrolo[2,3-b]pyridin-2-yl)methyl)-3-methylpiperazin-1-yl)-N,N-dimethylbenzamide (30 mg).

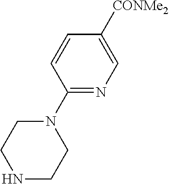

Boc-piperazine (0.40 g), 6-bromo-N,N-dimethylnicotinamide (0.59 g), NaOtBu (0.41 g), Xphos (0.10 g), and Pd₂dba₃ (0.1 g) were degassed in dioxane (4 mL) and stirred at 110° C. ON. The OL (water/EtOAc) was washed with brine, dried, concentrated, and purified by FC (pentane/EtOAc 1:1) to give 0.30 g of tert-butyl 4-(5-(dimethylcarbamoyl)pyridin-2-yl)-piperazine-1-carboxylate. 0.26 g of this material was stirred 1 h in 3M HCl in dioxane (3 mL) at 0° C. to RT and concentrated to give Int. 1M55 N,N-dimethyl-6-(piperazin-1-yl) nicotinamide (0.14 g, HCl salt).

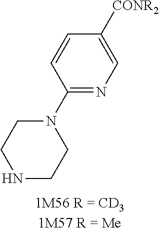

4-(4-tert-Butoxycarbonylpiperazin-1-yl)benzoic acid (1.0 g), HN(CD₃)₂ (0.43 g, HCl salt), HOBt (0.66 g), and EDC (0.94 g, HCl salt) were stirred ON in DCM/DIPEA (15.4:1, 21 mL). The OL (water/DCM) was washed with brine, dried, concentrated, and purified by FC (pentane/EtOAc 1:1) to give tert-butyl 4-(5-(bis(methyl-ds)carbamoyl)-pyridin-2-yl) piperazine-1-carboxylate (0.95 g). This material was stirred in DCM/TFA (6.7:1, 35 mL), concentrated, and triturated in Et₂O to give Int. 1M56 N,N-bis(methyl-d₃)-6-(piperazin-1-yl) nicotinamide (0.85 g). Int. 1AE20 (14 g) was hydrogenated ON using 10% Pd/C (3.5 g) in MeOH (200 mL), filtered, and concentrated to give tert-butyl 4-[5-(dimethyl-carbamoyl)-2-pyridyl] piperidine-1-carboxylate (11 g). 0.9 g of this material was stirred in DCM/TFA (10:1, 11 mL) at 0° C. to RT, concentrated, and triturated in Et₂O to give Int. 1M57 N,N-dimethyl-6-(piperidin-4-yl) nicotinamide (0.50 g, TFA salt).

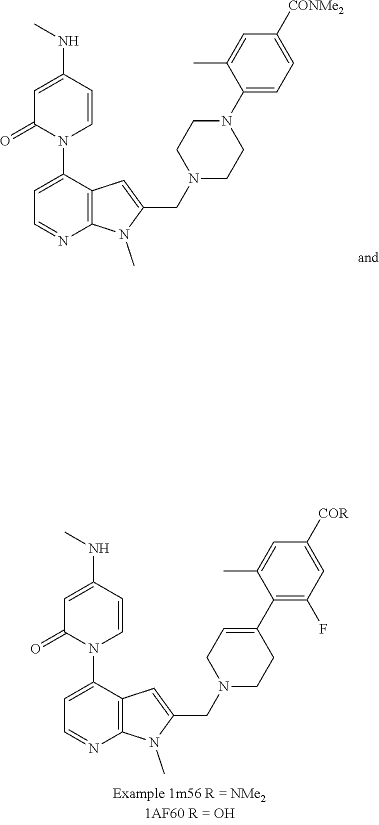

Ints. 1M1/1S7 (15 mg/22 mg, HCl salt) were stirred 0.5 h in DCE (0.5 mL). STAB (25 mg) was added and stirring was continued ON. The mixture was filtered. The filtrate was stirred 0.5 h in TFA (0.5 mL), concentrated, and purified by HPLC to give Example 1m6 N,N,3-Trimethyl-4-(4-((1-methyl-4-(4-(methylamino)-2-oxopyridin-1(2H)-yl)-1H-pyrrolo[2,3-b]pyridin-2-yl)methyl) piperazin-1-yl)benzamide (6.6 mM in DMSO (0.70 mL). Example 1m56 3-Fluoro-N,N,5-trimethyl-4-(1-((1-methyl-4-(4-(methylamino)-2-oxopyridin-1(2H)-yl)-1H-pyrrolo[2,3-b]pyridin-2-yl)methyl)-1,2,3,6-tetrahydropyridin-4-yl)benzamide (10 mM in DMSO (1.2 mL)) was prepared similarly to Example 1m6 from Int. 1M25 (17 mg). Example 1m56 (2.4 g) and LiOH—H₂O (1.1 g) were stirred 3 h in THF/water (2.5:1, 28 mL) at 0° C. to 60° C. The residue after concentration was triturated in aq. HCl to give Int. 1M60 3-fluoro-5-methyl-4-(1-((1-methyl-4-(4-(methylamino)-2-oxopyridin-1(2H)-yl)-1H-pyrrolo[2,3-b]pyridin-2-yl)methyl)-1,2,3,6-tetrahydropyridin-4-yl)benzoic acid (2.4 g).

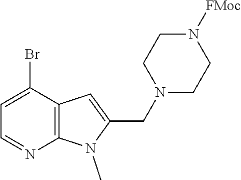

Int. 1AB8 (0.25 g) and 9H-fluoren-9-ylmethyl piperazine-1-carboxylate (0.48 g) were stirred 2 h in DCE/AcOH (100:1, 10 mL). STAB (0.44 g) was added and stirring continued 2 h at 0° C. to RT. The OL (sat. aq. NaHCO₃/DCM) was washed with brine, dried, and concentrated to give Int. 1N2 9H-fluoren-9-ylmethyl 4-[(4-bromo-1-methyl-pyrrolo[2,3-b]pyridin-2-yl)methyl]piperazine-1-carboxylate (0.20 g).

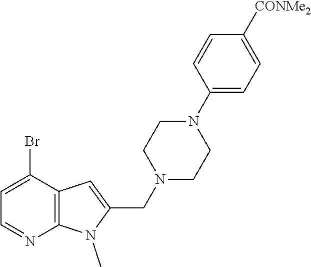

Int. 1AB8 (0.35 g) and N,N-dimethyl-4-(piperazin-1-yl)benzamide (0.40 g, HCl salt) were stirred 4 h in DCE/DIPEA (6.5:1, 11.6 mL). STAB (0.94 g) was added and stirring continued ON. The OL (water/DCM) was washed with brine, dried, concentrated, and purified by FC (hexane/EtOAc 1:4) to give Int. 1N4 4-(4-((4-bromo-1-methyl-1H-pyrrolo[2,3-b]pyridin-2-yl)methyl) piperazin-1-yl)-N,N-dimethyl-benzamide (0.44 g).

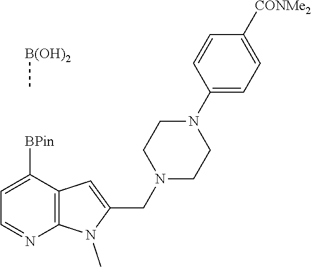

Int. 1N4 (25 mg), KOAc (11 mg), PdDPPFCl₂-DCM (4 mg), and B₂Pin₂(15 mg) were degassed in dioxane (3 mL) and stirred 4 h at 70° ch. KOAc (11 mg), and B₂Pin₂(15 mg), and PdDPPFCl₂-DCM (4 mg) were added and stirring continued 4 h at 70° C. and ON at RT. KOAc (11 mg), B₂Pin₂(15 mg), and PdDPPFCl₂-DCM (4 mg) were added and stirring continued 6 h at 90° C. The mixture was filtered and HPLC-purified to give Int. 1N5 N,N-dimethyl-4-[4-[[1-methyl-4-(4,4,5,5-tetramethyl-1,3,2-dioxa-borolan-2-yl) pyrrolo[2,3-b]pyridin-2-yl]methyl]piperazin-1-yl]benzamide and (2-((4-(4-(dimethyl-carbamoyl)-phenyl) piperazin-1-yl)methyl)-1-methyl-1H-pyrrolo[2,3-b]pyridin-4-yl) boronic acid.

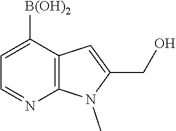

Int. 1J3 (1.0 g), B₂Pin₂(1.58 g), Pd(PPh₃)₂Cl₂ (0.29 g), and KOAc (1.02 g) were degassed dioxane (10 mL) and stirred 3 h at 100° C. and concentrated. The residue decanted with pentane and concentrated to give Int. 1O3 [2-(hydroxymethyl)-1-methyl-pyrrolo[2,3-b]pyridin-4-yl]boronic acid (0.8 g).

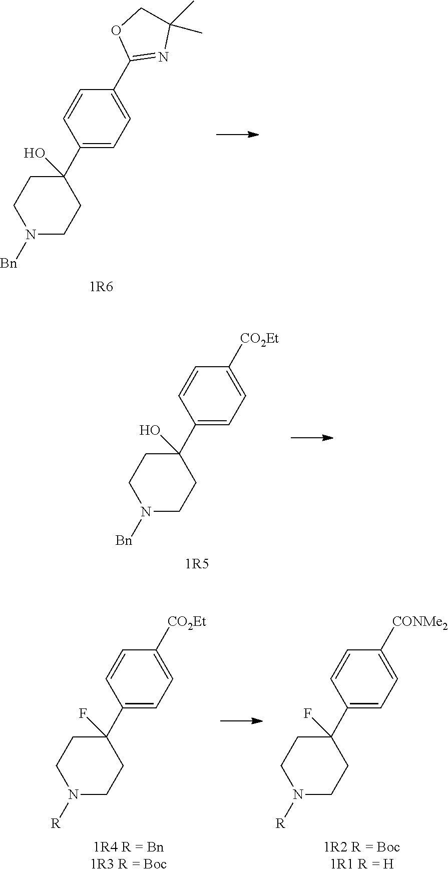

2-(4-Bromophenyl)-4,4-dimethyl-4,5-dihydrooxazole (10 g) and Mg turnings (1.1 g) and I₂ (50 mg) were refluxed 1 h in THF (50 mL). 1-Benzylpiperidin-4-one (8.0 mL) was added before refluxing 3 h. The OL (sat. aq. NH₄Cl/MTBE) was washed with water, brine, dried, and concentrated to give Int. 1R6 1-benzyl-4-(4-(4,4-dimethyl-4,5-dihydro-oxazol-2-yl)phenyl) piperidin-4-ol. Int. 1R6 (7.0 g) was stirred ON in EtOH/96% H₂SO₄ (10:1, 400 mL) at 90° C. and concentrated. The OL (sat. aq. NaHCO₃/DCM) was dried, concentrated, and purified by FC (DCM/2M NH₃ in MeOH 1:0 to 4:1) to give Int. 1R5 ethyl 4-(1-benzyl-4-hydroxypiperidin-4-yl)-benzoate. Int. 1R5 (0.06 g) was dissolved in DCM (10 mL) at −78° C. DAST (0.28 mL) was added and stirring continued 2 h at −78° C. to RT over 2 h. The OL (DCM/water) was washed with brine, dried, concentrated, and HPLC-purified to give Int. 1R4 ethyl 4-(1-benzyl-4-fluoro-piperidin-4-yl)benzoate. Int. 1R4 (2.0 g) was stirred 3 h in DCM (40 mL) and 1-chloroethyl carbono-chloridate (0.76 mL) at 0° C. to RT and concentrated. The residue was refluxed 0.5 h in MeOH (20 mL) and concentrated. The residue and Boc₂O (1.56 g) were stirred ON in DCM/Et₃N (14.7:1, 27 mL). The OL (EtOAc/water) was washed with brine, dried, concentrated, and purified by FC (heptane/EtOAc 1:0 to 1:1) to give 4-(1-(tert-butoxycarbonyl)-4-fluoropiperidin-4-yl)benzoic acid. 0.77 g of this material, HATU (1.18 g), and HN(CH₃)₂ (0.29 g, HCl salt) were stirred 1 h in DMF/DIPEA (5:1, mL). The OL (EtOAc/water) was dried, concentrated, and purified by FC (heptane to EtOAc/MeOH 95:5) to give Int. 1R2 tert-butyl 4-(4-(dimethylcarbamoyl)-phenyl)-4-fluoropiperidine-1-carboxylate. Int. 1R2 (0.55 g) was stirred 1 h in MeOH/4M HCl in dioxane (1:1, 20 mL), concentrated, and purified by SCX and HPLC to give Int. 1R1 4-(4-fluoropiperidin-4-yl)-N,N-dimethylbenzamide (62 mg).

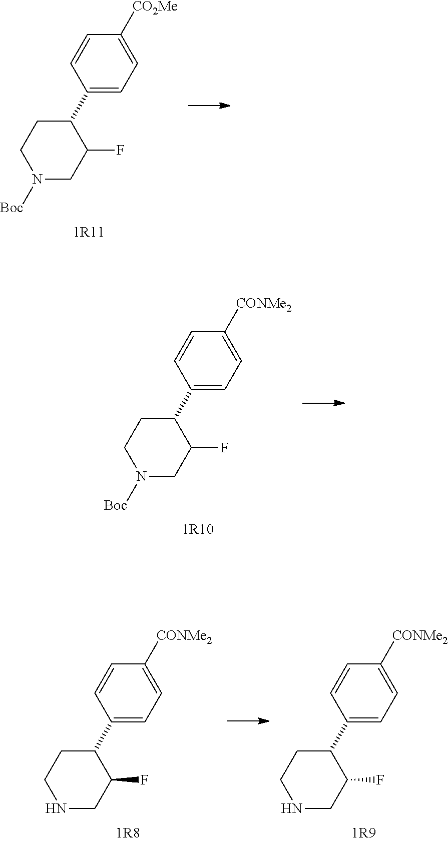

Int. 1AE14 (10.0 g) was stirred ON in THF/2.0 M BH₃-Me₂S in THF (11:6:1, 218 mL) at 0° C. to RT. 2.0 M aq. NaOH (38 mL) and 30% aq. H₂O₂ (5.9 mL) were added and stirring continued 1 h. The OL (sat. aq. Na₂S₂Oy EtOAc) was washed with water and brine, dried, concentrated, and purified by FC (hexane/EtOAc 7:3) to give tert-butyl 3-hydroxy-4-(4-(methoxycarbonyl)-phenyl) piperidine-1-carboxylate (7.4 g). 7.0 g of this material was dissolved in DCM (140 mL) at −78° C. 50% Deoxo-Fluor in THF (4.5 mL) was added and stirring continued 6 h at −78° C. to RT. The OL (sat. aq. NaHCO₃/DCM) were washed with sat. aq. citric acid, dried, concentrated, and purified by FC (hexane/EtOAc 85:15) to give Int. 1R11 tert-butyl 3-fluoro-4-(4-(methoxycarbonyl)phenyl)-piperidine-1-carboxylate (4.8 g). Int. 1R11 (10.0 g) and LiOH—H₂O (6.1 g) were stirred ON in MeOH/THF/water (2:4:1, 230 mL) at 0° C. to RT and concentrated. The OL (aq. citric acid/10% MeOH in DCM) was dried and concentrated to give 4-(1-(tert-butoxy-carbonyl)-3-fluoropiperidin-4-yl)benzoic acid (8.0 g). 22 g of this material, HATU (39 g), and HN(CH₃)₂ (16.5 g, HCl salt) were stirred ON in DMF/DIPEA (6:7:1, 0.46 L) at 0° C. to RT. The OL (water/MeOH/DCM (5:95)) was dried, concentrated, and purified by FC (hexane/EtOAc 2:3) to give tert-butyl 4-(4-(dimethylcarbamoyl)phenyl)-3-fluoropiperidine-1-carboxylate (20 g). This material was stirred ON in DCM/TFA (37:1, 513 mL) at 0° C. to RT ON and concentrated. The aq. layer (water/Et₂O) was basified with sat. aq. NaHCO₃ and extracted with MeOH/DCM (5:95). The OLs were dried and concentrated to give 4-(3-fluoropiperidin-4-yl)-N,N-dimethyl-benzamide (14.5 g). This material was resolved by SFC using a Chiralpak IG 250×25 5 μm column operated at 30° C. using an eluent of 70% CO₂ and 30% MeOH containing 0.5% HNEt₂ (100 g/min) and a back pressure of 100 bar to give Int. 1R8 (5.3 g, first peak) and Int. 1R9 (5.1 g, second peak) 4-((3R,4R)-3-fluoropiperidin-4-yl)-N,N-dimethylbenzamide and 4-((3S,4S)-3-fluoropiperidin-4-yl)-N,N-dimethylbenzamide. The absolute configurations of these compounds were not determined.

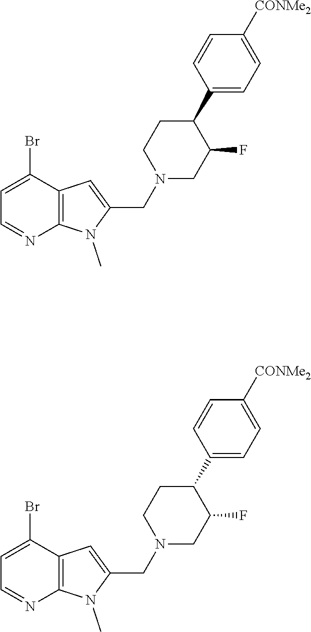

Int. 1F1 (0.10 g), DIPEA (0.21 mL), KI (20 mg), and Int. 1J1 were stirred ON in DMF (5 mL). The OL (EtOAc/water) was dried, concentrated, and purified by FC (hexane/EtOAc 3:7) to give Int. 1R12 N,N-Dimethyl-4-[(3R,4S)-1-[(4-bromo-1-methyl-pyrrolo[2,3-b]-pyridin-2-yl)methyl]-3-fluoro-4-piperidyl] benzamide (0.12 g). Int. 1R13 N,N-dimethyl-4-[(3S,4R)-1-[(4-bromo-1-methyl-pyrrolo[2,3-b]pyridin-2-yl)methyl]-3-fluoro-4-piperidyl] benzamide (0.12 g) was prepared similarly from Ints. 1F2/1J1 (0.10 g/0.16 g).

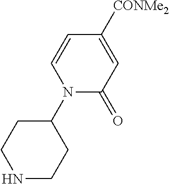

KOtBu (74 mg), K₂CO₃(0.17 g), N,N-dimethyl-2-oxo-1H-pyridine-4-carboxamide (0.10 g), and tert-butyl 4-(p-tolylsulfonyloxy) piperidine-1-carboxylate (0.24 g) were stirred ON in DME (5 mL) at 110° C. ON. The OL (EtOAc/water) was dried, concentrated, and purified by FC (hexane to EtOAc) to give tert-butyl 4-[4-(dimethyl-carbamoyl)-2-oxo-1-pyridyl] piperidine-1-carboxylate (70 mg). tert-Butyl 4-[4-(dimethylcarbamoyl)-2-oxo-1-pyridyl] piperidine-1-carboxylate (0.20 g) was stirred in DCM/TFA (10:1, 5.5 mL) and concentrated to give Int. 1S2 N,N-Dimethyl-2-oxo-1-(4-piperidyl) pyridine-4-carbox-amide and Int. 1S2′ N,N-dimethyl-2-(4-piperidyloxy) pyridine-4-carboxamide (0.15 g, TFA salt).

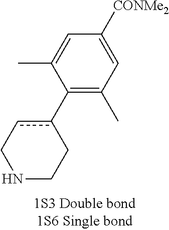

4-Bromo-N,N,3,5-tetramethylbenzamide (5.5 g), tert-butyl 4-(4,4,5,5-tetramethyl-1,3,2-dioxaborolan-2-yl)-3,6-dihydropyridine-1(2H)-carboxylate (7.3 g), PdDPPFCl₂-DCM (0.88 g), and NaHCO₃ (5.4 g) were degassed in dioxane/water (3:1, 47 mL) and stirred ON at 100° C. The OL (EtOAc/water) was dried, concentrated, and purified by FC (pentane/EtOAc 1:1) to give tert-butyl 4-[4-(dimethylcarbamoyl)-2,6-dimethyl-phenyl]-3,6-dihydro-2H-pyridine-1-carboxylate (4.5 g). 0.3 g of this material was stirred ON in 2.9M HCl in dioxane (7 mL) at 0° C. to R, concentrated, and triturated in Et₂O. The solid was treated with sat. aq. NaHCO₃ and concentrated. The residue was dissolved in DCM, dried, and concentrated to give Int. 1S3 N,N,3,5-tetramethyl-4-(1,2,3,6-tetrahydro-pyridin-4-yl)benzamide (0.15 g). tert-Butyl 4-[4-(dimethylcarbamoyl)-2,6-dimethyl-phenyl]-3,6-dihydro-2H-pyridine-1-carboxylate (50 mg) was hydrogenated 48 h using PtO₂ (15 mg) in AcOH (3 mL), filtered, and concentrated to give Int. 1S6 N,N,3,5-tetramethyl-4-(piperidin-4-yl)benzamide (35 mg).

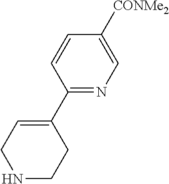

6-Bromo-N,N-dimethylnicotinamide 20.0 g), tert-butyl 4-(4,4,5,5-tetramethyl-1,3,2-dioxaborolan-2-yl)-3,6-dihydropyridine-1(2H)-carboxylate (27.0 g), NaHCO₃ (25.7 g), and PdDPPFCl₂-DCM (0.71 g) were degassed in dioxane/water (4:1, 0.5 L), refluxed ON, and filtered. The OL (water/EtOAc) was dried, concentrated, and purified by FC (pentane/EtOAc 45:55) to give tert-butyl 4-[5-(dimethyl-carbamoyl)-2-pyridyl]-3,6-dihydro-2H-pyridine-1-carboxylate (17 g). 1.0 g of this material was stirred ON in DCM/4M HCl in dioxane (2.6:1, 14 mL) at 0° C. to RT. The mixture was combined with another two batches prepared on 0.1 g scale and concentrated. The residue was stirred in water at pH 8 (adjusted with NaHCO₃). The mixture was purified by FC (reverse-phase C₁₈ column; 8% ACN in 0.001% aq. formic acid) to give Int. 1S4 N,N-dimethyl-1′,2′,3′,6′-tetrahydro-[2,4′-bipyridine]-5-carboxamide (0.60 g).

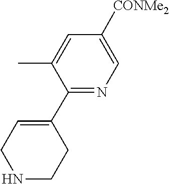

6-Bromo-N,N,5-trimethyl-pyridine-3-carboxamide (3.2 g), NaHCO₃ (3.9 g), PdDPPFCl₂-DCM (0.27 g), and tert-butyl 4-(4,4,5,5-tetramethyl-1,3,2-dioxaborolan-2-yl)-3,6-dihydropyridine-1(2H)-carboxylate (6.1 g) were degassed in dioxane/water (4:1, 13 mL) and stirred ON at 110° C. The mixture was filtered, concentrated, and purified by FC (hexane/EtOAc 1:4) to give tert-butyl 4-[5-(dimethyl-carba-moyl)-3-methyl-2-pyridyl]-3,6-dihydro-2H-pyridine-1-carboxylate (4.0 g). 0.35 g of this material was stirred ON in DCM/TFA (10:1, 9 mL) at 0° C. to RT, concentrated, and triturated in Et₂O/pentane to give Int. 1S5 N,N,3-trimethyl-1′,2′,3′,6′-tetrahydro-[2,4′-bipyridine]-5-carboxamide (0.35 g, TFA salt).

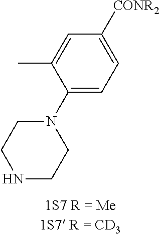

tert-Butyl piperazine-1-carboxylate (5.0 g), 4-bromo-N,N,3-trimethylbenzamide (7.1 g), Pd₂dba₃ (1.19 g), XPhos (1.29 g), and NaOtBu (5.19 g) were degassed in dioxane (50 mL) and stirred ON at 100° C. and filtered. The OL (water/EtOAc) was dried, concentrated, and purified by FC (pentane/EtOAc 3:7) to give tert-butyl 4-[4-(dimethyl-carbamoyl)-2-methyl-phenyl]piperazine-1-carboxylate (6.1 g). 7.2 g of this material was stirred ON in 4M HCl in dioxane/DCM (1.8:1, 55 mL) at 0° C. to RT, concentrated, and triturated in Et₂O/pentane to give Int. 1S7 N,N,3-trimethyl-4-(piperazin-1-yl)benzamide (5.0 g, HCl salt).

Int. 1S7′ 3-methyl-N,N-bis(methyl-d₃)-4-(piperazin-1-yl)benzamide was prepared similarly from 4-bromo-3-methyl-N,N-bis(methyl-d₃) benzamide.

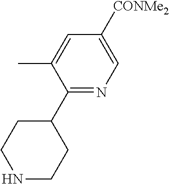

6-Bromo-N,N,5-trimethyl-pyridine-3-carboxamide (3.2 g), NaHCO₃ (3.9 g), and PdDPPFCl₂-DCM (0.27 g), tert-butyl 4-(4,4,5,5-tetramethyl-1,3,2-dioxaborolan-2-yl)-3,6-dihydropyridine-1(2H)-carboxylate (6.1 g) were degassed in dioxane/water (4:1, 14 mL) and stirred ON at 110° C. The mixture was filtered, concentrated, and purified by FC (hexane/EtOAc 1:4) to give tert-butyl 4-[5-(dimethylcarba-moyl)-3-methyl-2-pyridyl]-3,6-dihydro-2H-pyridine-1-carboxylate (4.0 g). 4.2 g of this material was hydrogenated ON using 10% Pd/C (2.0 g) in MeOH (100 mL), filtered, concentrated, and purified by FC (hexane/EtOAc 1:9) to give tert-butyl 4-[5-(dimethyl-carbamoyl)-3-methyl-2-pyridyl] piperidine-1-carboxylate (3.5 g). 4.3 g of this material was stirred ON in DCM/TFA (1.5:1, 25 mL) at 0° C. to RT, concentrated, and triturated in pentane/Et₂O to give Int. 1S8 N,N,5-Trimethyl-6-(piperidin-4-yl) nicotinamide (5.0 g, TFA salt).

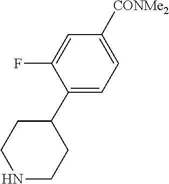

4-Bromo-3-fluoro-N,N-dimethyl-benzamide (1.0 g), tert-butyl 4-(4,4,5,5-tetramethyl-1,3,2-dioxaborolan-2-yl)-3,6-dihydropyridine-1(2H)-carboxylate (1.51 g), Na₂CO₃(1.72 g), and PdDPPFCl₂-DCM (0.17 g) were degassed in dioxane/water (4:1, 10 mL) and stirred ON at 100° C. The OL (water/EtOAc) was washed with brine, dried, concentrated, and purified by FC (hexane to EtOAc) to give tert-butyl 4-[4-(dimethyl-carbamoyl)-2-fluoro-phenyl]-3,6-dihydro-2H-pyridine-1-carboxylate (1.20 g). This material was hydrogenated ON using 10% Pd/C (45 mg) in MeOH (2 mL), filtered, concentrated, stirred ON in 4M HCl in dioxane (10 mL), concentrated, and HPLC-purified to give Int. 1S18 3-fluoro-N,N-dimethyl-4-(piperidin-4-yl)benzamide (63 mg).

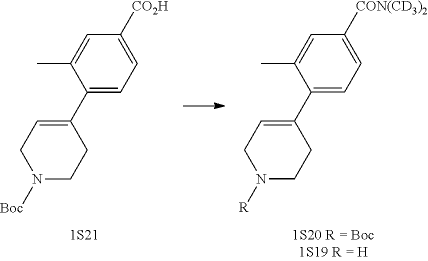

96% aq. H₂SO₄ (2.5 mL) was added to a solution of 4-bromo-3-methyl-benzoic acid (20 g) in MeOH (300 mL). The mixture was stirred at 90° C. ON and concentrated. The organic layer (sat. aq. NaHCO/EtOAc) was dried and concentrated to give methyl 4-bromo-3-methyl-benzoate (20 g). 10 g of this material, tert-butyl 4-(4,4,5,5-tetramethyl-1,3,2-dioxaborolan-2-yl)-3,6-dihydropyridine-1(2H)-carboxylate (14.8 g), Na₂CO₃(13.8 g), and PdDPPFCl₂-DCM (0.84 g) were degassed in dioxane/water (2.3:1, 100 mL), stirred ON at 90° C., filtered, concentrated, and purified by FC (hexane/EtOAc 95:5) to give tert-butyl 4-(4-methoxycarbonyl-2-methyl-phenyl)-3,6-dihydro-2H-pyridine-1-carboxylate (7.0 g). 2.5 g of this material and LiOH—H₂O (2.0 g) were stirred ON in EtOH/THF/water (2:1:1, 24 mL) at 0° C. to RT, concentrated, and triturated in dilute aq. citric acid to give Int. 1S21 4-(1-tert-butoxycarbonyl-3,6-dihydro-2H-pyridin-4-yl)-3-methyl-benzoic acid (2.0 g). Int. 1S20 (0.70 g) was prepared similarly to Int. 1D8 from Int. 1S21 (2.0 g). Int. 1S19 3-methyl-N,N-bis(methyl-d₃)-4-(1,2,3,6-tetrahydropyridin-4-yl)benzamide (0.31 g) was prepared similarly to Int. 1S7 from Int. 1S20 (0.80 g).

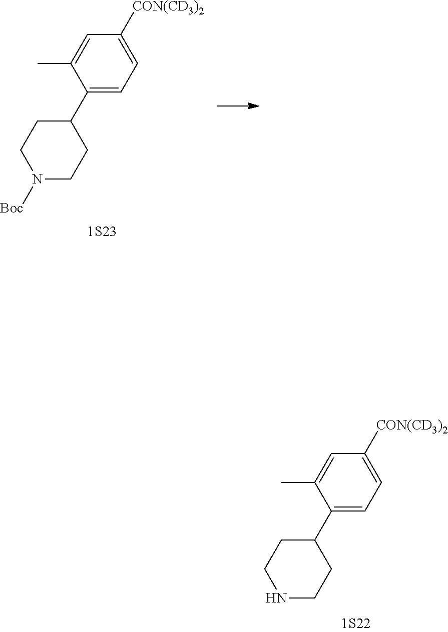

Int. 1S20 (0.35 g) was hydrogenated ON using 10% Pd/C (50 mg) in MeOH (10 mL), filtered, and concentrated to give Int. 1S23 (0.21 g). Int. 1S22 3-methyl-N,N-bis-(methyl-d₃)-4-(piperidin-4-yl)benzamide (0.45 g) was prepared similarly to Int. 1S7 from Int. 1S23 (0.90 g).

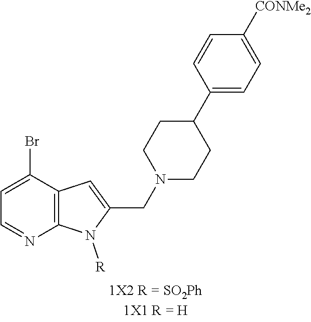

Int. 1A6 (0.30 g), N,N-dimethyl-4-(4-piperidyl)benzamide (0.34 g), and 4 Å MS were stirred 0.3 h in DCM (5 mL). STAB (0.52 g) was added and stirring was continued ON. The mixture was filtered, concentrated, and purified by FC (heptane to EtOAc/MeOH 1:4) to give Int. 1×2 4-[1-[[1-(benzenesulfonyl)-4-bromo-pyrrolo-[2,3-b]-pyridin-2-yl]-methyl]-4-piperidyl]-N,N-dimethyl-benzamide (0.22 g). Int. 1×2 (0.12 g) was stirred 2.5 h in EtOH/4M aq. NaOH (2.5:1, 2.8 mL) at 80° C. and concentrated. The OL (water/DCM) was concentrated to give Int. 1×1 4-[1-[(4-bromo-1H-pyrrolo[2,3-b]pyridin-2-yl)methyl]-4-piperidyl]-N,N-dimethyl-benzamide (94 mg).

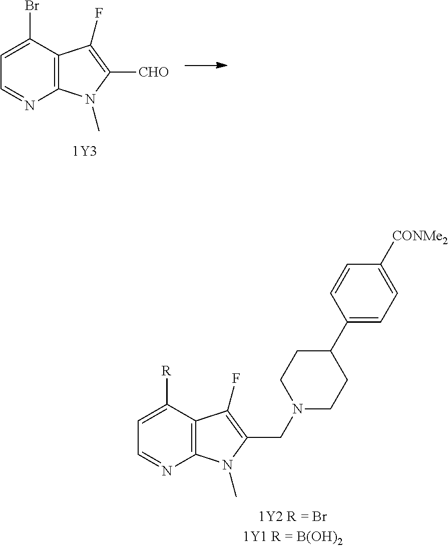

4-Bromo-3-fluoro-1-methyl-pyrrolo[2,3-b]pyridine (1.9 g) was stirred 2 h in THF/2.0 M LDA in THF (5:1, 24 mL) at −78° C. DMF (1.6 mL in THF (5 mL)) was added and stirring continued 2 h at −78° C. to RT. The OL (water/DCM) was washed with brine, dried, concentrated, and purified by FC (pentane/EtOAc 1:4) to give Int. 1Y^{3 4}-bromo-3-fluoro-1-methyl-pyrrolo[2,3-b]pyridine-2-carbaldehyde (0.4 g). Int. 1Y³ (0.39 g) and N,N-dimethyl-4-(4-piperidyl)benzamide trifluoro acetate (0.38 g) were stirred ON in DCE/DIPEA (3.8:1, 6.3 mL) at 0° C. to RT. STAB (0.64 g) was added and stirring continued ON at 0° C. to RT. The OL (EtOAc/water) was washed brine, dried, concentrated, and purified by FC (pentane to EtOAc) to give Int. 1Y2 4-[1-[(4-bromo-3-fluoro-1-methyl-pyrrolo[2,3-b]pyridin-2-yl)methyl]-4-piperidyl]-N,N-dimethyl-benzamide (0.38 g). Int. 1Y² (0.25 g), B₂Pin₂(0.17 g), KOAc (0.16 g), and PdDPPFCl₂-DCM (37 mg) were degassed in THF (20 mL) and ON at 100° C., concentrated, and triturated in pentane to give Int. 1Y¹ (2-((4-(4-(dimethylcarbamoyl)phenyl)-piperidin-1-yl)methyl)-3-fluoro-1-methyl-1H-pyrrolo[2,3-b]pyridin-4-yl) boronic acid (0.2 g).

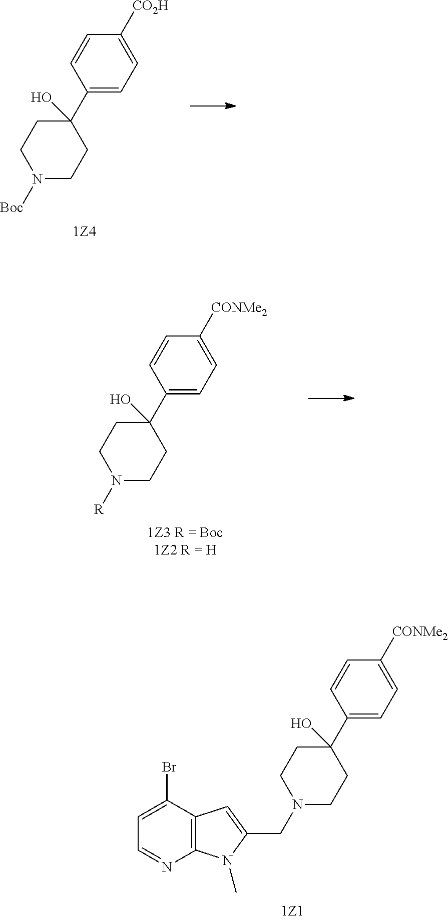

Int. 1R5 (0.5 g) and LiOH (181 mg) were stirred ON in MeOH/THF/water (3:5:1, 9 mL) and concentrated to give Int. 1Z4 4-(1-benzyl-4-hydroxypiperidin-4-yl)benzoic acid (0.40 g). Int. 1Z4 (0.40 g), HN(CH₃)₂ (0.31 g, HCl salt), and HATU (636 mg) were stirred ON in DMF/DIPEA (7:3:1, 9.1 mL). The OL (EtOAc/water) was concentrated, and purified by FC (heptane/EtOAc 5:3 to 3:7) to give Int. 1Z3 4-(1-benzyl-4-hydroxy-piperidin-4-yl)-N,N-dimethyl-benzamide. Int. 1Z3 (1.0 g) was hydrogenated 32 h using 10% Pd/C (0.5 g) in MeOH (20 mL), filtered, and concentrated to give Int. 1Z2 4-(4-hydroxypiperidin-4-yl)-N,N-dimethylbenzamide. Int. 1Z2 (0.20 g) and 4-bromo-1H-pyrrolo[2,3-b]pyridine-2-carboxaldehyde (0.19 g) were stirred 4 h in DCE/DIPEA (10:1, 3.3 mL). STAB (0.51 g) was added and stirring continued 32 h. The OL (water/DCM) was concentrated and purified by FC (hexane/EtOAc 1:0 to 4:1) to give Int. 1Z1 4-[1-[(4-Bromo-1-methyl-pyrrolo[2,3-b]pyridin-2-yl)methyl]-4-hydroxy-4-piperidyl]-N,N-dimethyl-benzamide (0.15 g).

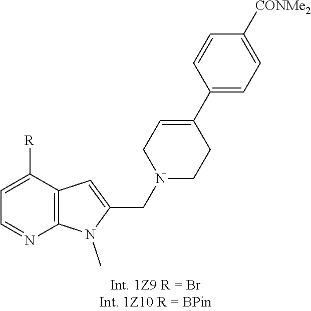

Int. 1J1 (0.97 g), KI (12 mg), Int. 1AE7 (1 g), and K₂CO₃(1.6 g) were stirred 11 h in DMF (10 mL). The OL (water/MTBE) was concentrated to give Int. 1Z9 4-(1-((4-bromo-1-methyl-1H-pyrrolo[2,3-b]pyridin-2-yl)methyl)-1,2,3,6-tetrahydropyridin-4-yl)-N,N-dimethylbenzamide (0.67 g). Int. 1Z9 (0.67 g), KOAc (0.44 g), PdDPPFCl₂-DCM (24 mg), and bis (pinacolato)diboron (0.75 g) were stirred 48 h in dioxane (10 mL) at 90° C. under an inert atmosphere. The OL (water/MTBE) was concentrated to give Int. 1Z10 N,N-dimethyl-4-(1-((1-methyl-4-(4,4,5,5-tetramethyl-1,3,2-dioxaborolan-2-yl)-1H-pyrrolo-[2,3-b]pyridin-2-yl)methyl)-1,2,3,6-tetrahydropyridin-4-yl) benzamide (0.72 g). 6-bromopyridazin-3-amine (0.25 g) and di-tert-butyl dicarbonate (0.38 g) were stirred ON in 1M LiHMDs in THF (1.9 mL) and THF (5 mL) at 0° C. to RT under an inert atmosphere. The OL (sat. aq. NH₄Cl/EtOAc) was dried, and concentrated to give Int. 1Z11 tert-butyl (6-bromopyridazin-3-yl)carbamate (0.15 g).

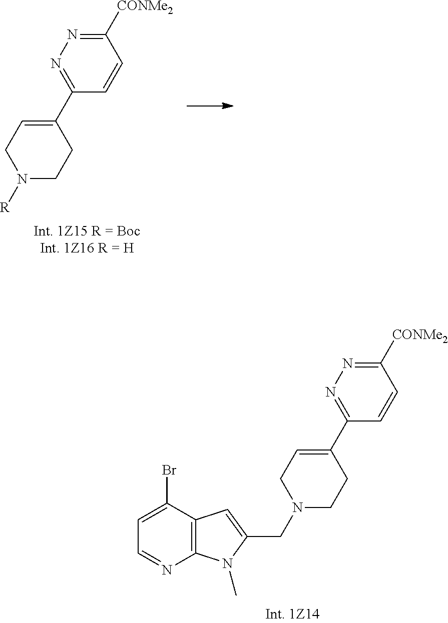

6-Chloro-N,N-dimethylpyridazine-3-carboxamide (0.85 g), tert-butyl 4-(4,4,5,5-tetramethyl-1,3,2-dioxaborolan-2-yl)-3,6-dihydropyridine-1(2H)-carboxylate (1.6 g), K₂CO₃(2.5 g), and PdDPPFCl₂-DCM (0.19 g) were stirred ON in dioxane/H₂O (4:1, 25 mL) at 100° C. under an inert atmosphere. The OL (water/EtOAc) was dried, concentrated, and purified by FC (CHCl₃//MTBE 1:0 to 0:1) to give Int. 1Z15 tert-butyl 4-(6-(dimethylcarbamoyl) pyridazin-3-yl)-3,6-dihydropyridine-1(2H)-carboxylate (1 g). Int. 1Z15 (1 g) was stirred 1 h in 1,1,1,3,3,3-hexafluoro-2-propanol/10M HCl (19:1, 10.5 mL). The mixture was diluted with MTBE to precipitate Int. 1Z16 N,N-dimethyl-6-(1,2,3,6-tetrahydro-pyridin-4-yl) pyridazine-3-carboxamide (0.8 g, HCl salt). Ints. 1J1/1Z16 (0.2 g/0.3 g) were stirred 12 h in ACN/Et₃N (48:1, 20.4 mL). The mixture was concentrated and HPLC-purified to give Int. 1Z14 6-(1-((4-bromo-1-methyl-1H-pyrrolo[2,3-b]pyridin-2-yl)methyl)-1,2,3,6-tetrahydropyridin-4-yl)-N,N-dimethylpyridazine-3-carboxamide (70 mg).

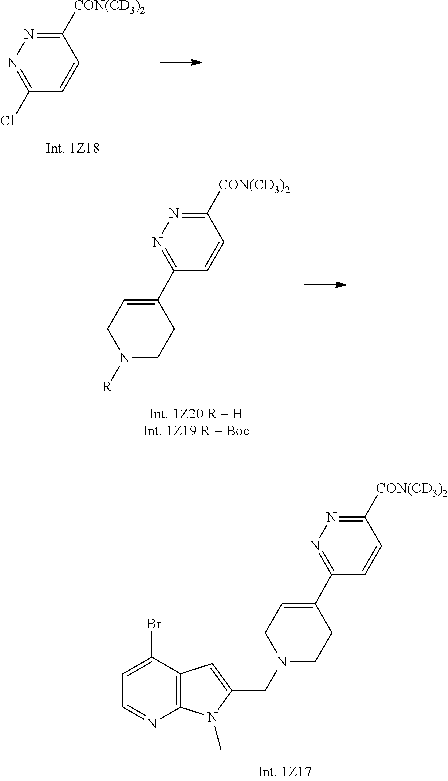

6-Chloropyridazine-3-carboxylic acid (1 g) was stirred in DCM/SOCl₂(40:1, 20.5 mL) for 2 h at 40° C. and concentrated. Bis (methyl-d₃) amine hydrochloride (0.61 g) in DCM (20 mL) and Et₃N (2.6 mL) were added and the mixture was stirred ON. The mixture was concentrated and purified by FC (CHCl₃/ACN 1:0 to 1:1) to give Int. 1Z18 6-chloro-N,N-bis(methyl-d₃) pyridazine-3-carboxamide (1.2 g). Int. 1Z18 (1.2 g), tert-butyl 4-(4,4,5,5-tetramethyl-1,3,2-dioxaborolan-2-yl)-3,6-dihydropyridine-1(2H)-carboxylate (2.1 g), K₂CO₃(3.5 g), and PdDPPFCl₂-DCM (0.26 g) were stirred ON in dioxane/H₂O (4:1, 25 mL) at 100° C. under an inert atmosphere. The OL (water/EtOAc) was dried, concentrated, and purified by FC (CHCl₃/MTBE 1:0 to 0:1) to give Int. 1Z19 tert-butyl 4-(6-(bis-(methyl-d₃)carbamoyl)-pyridazin-3-yl)-3,6-dihydropyridine-1(2H)-carboxylate (0.55 g). Int. 1Z19 (0.55 g) was stirred 1 h in 1,1,1,3,3,3-hexafluoro-2-propanol/10M HCl (26:1, 5.2 mL). The mixture was diluted with MTBE to precipitate Int. 1Z20 N,N-bis(methyl-d₃)-6-(1,2,3,6-tetrahydropyridin-4-yl)-pyridazine-3-carboxamide (0.2 g, HCl salt). Ints. 1J1/1Z20 (0.2 g/0.2 g) were stirred 12 h in ACN/Et₃N (48:1, 20.4 mL). The mixture was concentrated and HPLC-purified to give Int. 1Z17 6-(1-((4-bromo-1-methyl-1H-pyrrolo[2,3-b]-pyridin-2-yl)methyl)-1,2,3,6-tetrahydropyridin-4-yl)-N,N-bis(methyl-d₃) pyridazine-3-carboxamide (50 mg).

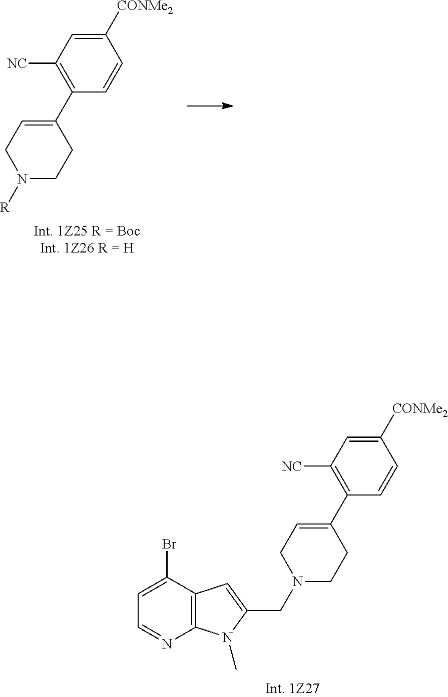

4-Chloro-3-cyanobenzoic acid (3.4 g) and di (1H-imidazol-1-yl) methanone (3.0 g) were refluxed 0.3 h in dioxane (50 mL). Dimethylamine hydrochloride (1.5 g) was added and stirring continued 10 h. The mixture was concentrated. The OL (aq. NaHSO₄/CHCl₃) was concentrated to give Int. 1Z24 4-chloro-3-cyano-N,N-dimethyl-benzamide (0.87 g). Int. 1Z24 (1 g), tert-butyl 4-(4,4,5,5-tetramethyl-1,3,2-dioxaborolan-2-yl)-3,6-dihydropyridine-1(2H)-carboxylate (1.6 g), K₂CO₃(2.0 g), and PdDPPFCl₂-DCM (0.20 g) were stirred in dioxane/H₂O (4:1, 20 mL) ON at 100° C. under an inert atmosphere. The mixture was filtered, concentrated, and purified by FC (CHCl₃/ACN 1:0 to 4:1) to give Int. 1Z25 tert-butyl 4-(2-cyano-4-(dimethyl-carbamoyl)phenyl)-3,6-dihydropyridine-1(2H)-carboxylate (0.44 g). Int. 1Z25 (0.44 g) was stirred ON in 2.8M HCl in dioxane (10 mL). The mixture was concentrated to give Int. 1Z26 3-cyano-N,N-dimethyl-4-(1,2,3,6-tetrahydropyridin-4-yl)benzamide (0.3 g, HCl salt). Ints. 1J1/1Z26 (0.3 g/0.3 g) and K₂CO₃(0.5 g) were stirred 11 h in DMF (10 mL). The mixture was diluted with water to precipitate Int. 1Z27 4-(1-((4-bromo-1-methyl-1H-pyrrolo[2,3-b]pyridin-2-yl)methyl)-1,2,3,6-tetrahydropyridin-4-yl)-3-cyano-N,N-dimethylbenzamide (0.12 g).

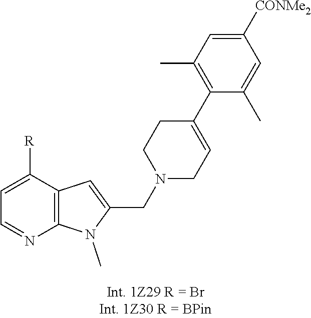

Ints. 1J1/RR44 (0.3 g/0.3 g) were stirred 12 h in ACN/Et₃N (29:2, 10.7 mL). The mixture was concentrated and HPLC-purified to give Int. 1Z29 4-(1-((4-bromo-1-methyl-1H-pyrrolo[2,3-b]pyridin-2-yl)methyl)-1,2,3,6-tetrahydropyridin-4-yl)-N,N,3,5-tetramethylbenzamide (0.3 g). Int. 1Z29 (0.3 g), bis (pinacolato)diboron (0.4 g), KOAc (0.5 g), and PdDPPFCl₂-DCM (50 mg) were stirred 12 h in dioxane (10 mL) at 80° C. under an inert atmosphere. The OL (water/EtOAc) was concentrated to give Int. 1Z30 N,N,3,5-tetramethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.3 g).

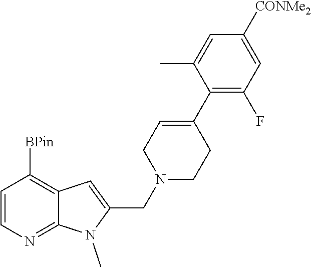

Int. 1Z50 (0.75 g), bis (pinacolato)diboron (0.78 g), PdDPPFCl₂-DCM (0.13 g), and KOAc (0.45 g) were stirred 1.3 h in dioxane (10 mL) at 110° C. under an inert atmosphere under MW conditions. The mixture was filtered through celite and concentrated to give Int. 1Z31 3-fluoro-N,N,5-trimethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.85 g).

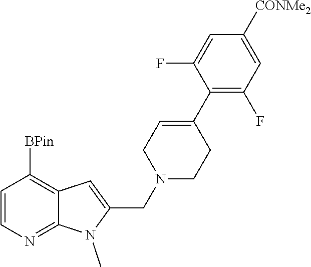

Int. 1Z65 (0.50 g), bis (pinacolato)diboron (0.52 g), PdDPPFCl₂-DCM (83 mg), and KOAc (0.30 g) were stirred 1 h in dioxane (10 mL) at 110° C. under an inert atmosphere under MW conditions. The mixture was filtered through celite, concentrated, and washed with pentane to give Int. 1Z33 3,5-difluoro-N,N-dimethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.50 g).

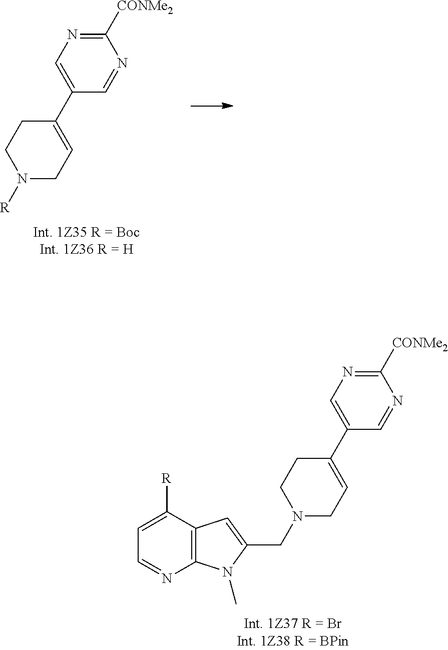

5-Bromo-N,N-dimethylpyrimidine-2-carboxamide (1.3 g), tert-butyl 4-(4,4,5,5-tetramethyl-1,3,2-dioxaborolan-2-yl)-3,6-dihydropyridine-1(2H)-carboxylate (1.9 g), K₂CO₃ (2.3 g), and PdDPPFCl₂-DCM (0.22 g) were stirred ON in dioxane/H₂O (4:1, 20 mL) at 100° C. under an inert atmosphere. The mixture was concentrated and purified by FC (MTBE/MeOH) to give Int. 1Z35 tert-butyl 4-(2-(dimethylcarbamoyl) pyrimidin-5-yl)-3,6-dihydropyridine-1(2H)-carboxylate (0.9 g). Int. 1Z35 (0.9 g) was stirred ON in 2.8M HCl in dioxane (10 mL). The mixture was concentrated to give Int. 1Z36 N,N-dimethyl-5-(1,2,3,6-tetrahydropyridin-4-yl)pyrimidine-2-carboxamide (0.65 g, HCl salt). Ints. 1J1/1Z36 (0.3 g/0.3 g) and K₂CO₃ (0.5 g) were stirred 11 h in DMF (10 mL). The mixture was diluted with water to precipitate Int. 1Z37 5-(1-((4-bromo-1-methyl-1H-pyrrolo[2,3-b]pyridin-2-yl)methyl)-1,2,3,6-tetrahydropyridin-4-yl)-N,N-dimethylpyrimidine-2-carboxamide (0.35 g). Int. 1Z37 (0.35 g), bis (pinacolato)diboron (0.70 g), PdDPPFCl₂-DCM (10 mg), and KOAc (0.40 g) were stirred 14 h in dioxane (10 mL) at 90° C. under an inert atmosphere. The OL (water/chloroform) was concentrated and HPLC-purified to give Int. 1Z38 N,N-dimethyl-5-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)pyrimidine-2-carboxamide (90 mg).

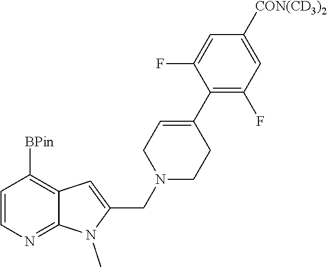

Int. 1Z65 (0.50 g), bis (pinacolato)diboron (0.52 g), PdDPPFCl₂-DCM (82 mg), and KOAc (0.30 g) were stirred 1 h in dioxane (6 mL) at 120° C. under MW conditions. The mixture was filtered through celite and concentrated to give Int. 1Z40 3,5-difluoro-N,N-bis(methyl-d₃)-4-(1-((1-methyl-4-(4,4,5,5-tetra-methyl-1,3,2-dioxaborolan-2-yl)-1H-pyrrolo[2,3-b]pyridin-2-yl)methyl)-1,2,3,6-tetrahydropyridin-4-yl)benzamide (0.60 g).

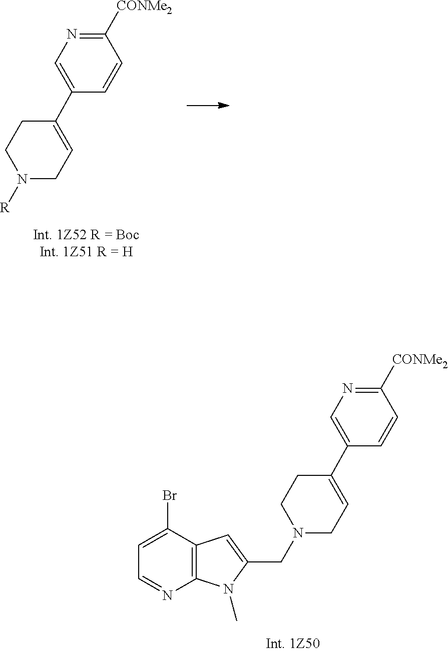

5-Bromo-N,N-dimethylpicolinamide (1.5 g), tert-butyl 4-(4,4,5,5-tetramethyl-1,3,2-dioxaborolan-2-yl)-3,6-dihydropyridine-1(2H)-carboxylate (2.2 g), K₂CO₃ (2.7 g), and PdDPPFCl₂-DCM (0.27 g) were stirred ON in dioxane/H₂O (4:1, 20 mL) ON 100° C. under an inert atmosphere. The mixture was concentrated and purified by FC (hexane/MTBE 1:0 to 0:1) to give Int. 1Z52 tert-butyl 6-(dimethyl-carbamoyl)-3′, 6′-dihydro-[3,4′-bipyridine]-1′ (2′H)-carboxylate (1 g). Int. 1Z51 (1 g) was stirred ON in 2.8M HCl in dioxane (10 mL). The mixture was concentrated to give Int. 1Z52 N,N-dimethyl-1′,2′,3′,6′-tetrahydro-[3,4′-bipyridine]-6-carboxamide (0.5 g, HCl salt). Ints. 1J1/1Z52 (0.6 g/0.5 g) and K₂CO₃ (0.9 g) were stirred 11 h in DMF (10 mL). The mixture was diluted with water to precipitate Int. 1Z50 1′-((4-bromo-1-methyl-1H-pyrrolo[2,3-b]pyridin-2-yl)methyl)-N,N-dimethyl-1′,2′,3′,6′-tetrahydro-[3,4′-bipyridine]-6-carboxamide (0.4 g).

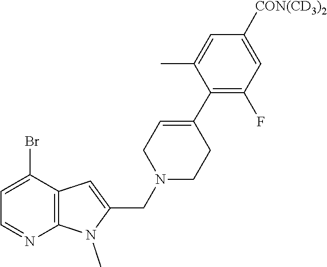

Int. 1AE9 (0.45 g), bis (pinacolato)diboron (0.35 g), PdDPPFCl₂-DCM (75 mg), and KOAc (0.27 g) were stirred 1 h in dioxane (5 mL) for 1 h at 110° C. under MW conditions. The mixture was filtered through celite and concentrated to give Int. 1Z53 3-fluoro-5-methyl-N,N-bis(methyl-d₃)-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.40 g).

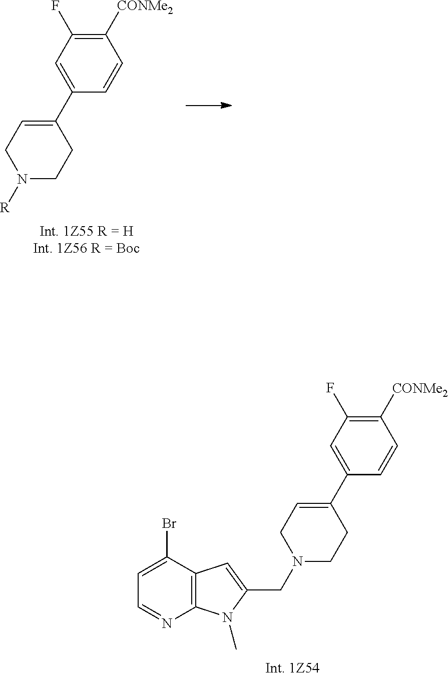

PdDPPFCl₂-DCM (0.25 g), 4-bromo-2-fluoro-N,N-dimethylbenzamide (1.5 g), tert-butyl 4-(4,4,5,5-tetramethyl-1,3,2-dioxaborolan-2-yl)-3,6-dihydropyridine-1(2H)-carboxylate (2.1 g), and K₂CO₃ (2.5 g) were stirred ON in dioxane/H₂O (4:1, 20 mL) at 100° C. under an inert atmosphere. The mixture was concentrated and purified by FC (hexane/MTBE 3:2 to 0:1) to give Int. 1Z55 tert-butyl 4-(4-(dimethyl-carbamoyl)-3-fluorophenyl)-3,6-dihydropyridine-1(2H)-carboxylate (1 g). Int. 1Z55 (1 g) was stirred ON in 2.8M HCl in dioxane (10 mL). The mixture was concentrated to give Int. 1Z56 2-fluoro-N,N-dimethyl-4-(1,2,3,6-tetrahydropyridin-4-yl)benzamide (0.5 g, HCl salt). Ints. 1J1/PP60 (0.5 g/0.5 g) and K₂CO₃ (0.8 g) were stirred 11 h in DMF (10 mL). The mixture was diluted with water to precipitate Int. 1Z54 4-(1-((4-bromo-1-methyl-1H-pyrrolo[2,3-b]pyridin-2-yl)methyl)-1,2,3,6-tetra-hydropyridin-4-yl)-2-fluoro-N,N-dimethylbenzamide (0.4 g).

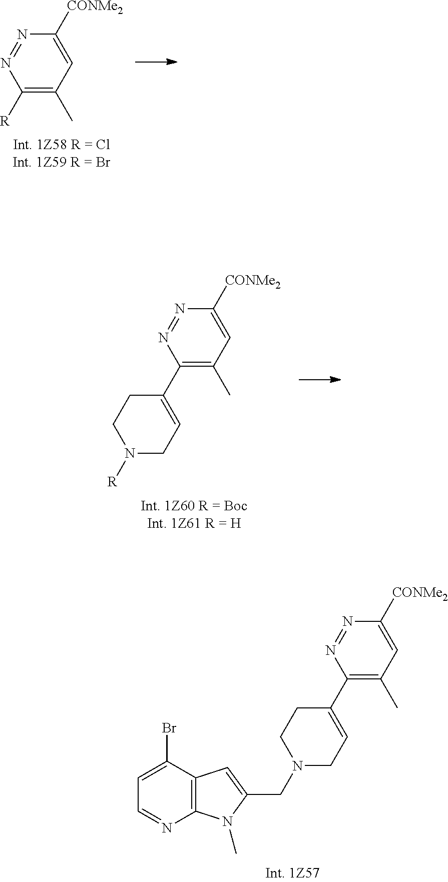

CDI (2.6 g) in THF (175 mL) was added dropwise to a mixture of 6-chloro-5-methylpyridazine-3-carboxylic acid (2 g) in THF (175 mL). The mixture was refluxed 3.5 h. Dimethylamine hydrochloride (2.8 g) and Et N (5.2 mL) were added and stirring continued 16 h at RT. The OL (brine/EtOAc) was concentrated to give Int. 1Z58 6-chloro-N,N,5-trimethylpyridazine-3-carboxamide (0.8 g). Int. 1Z58 was refluxed ON in bromotrimethylsilane (10 mL). The mixture was concentrated and purified by preparative TLC (hexane/EtOAc, 3:1) to give Int. 1Z59 6-bromo-N,N,5-trimethylpyridazine-3-carbox-amide (0.9 g). PdDPPFCl₂-DCM (0.3 g), Int. 1Z59 (0.9 g), tert-butyl 4-(4,4,5,5-tetramethyl-1,3,2-dioxa-borolan-2-yl)-3,6-dihydropyridine-1(2H)-carboxylate (1.0 g), and K₂CO₃ (2.0 g) were refluxed ON in dioxane/H₂O (15:4, 190 mL) under an inert atmosphere. The OL (aq. NaHCO₃/EtOAc) was dried, and concentrated to give Int. 1Z60 tert-butyl 4-(6-(dimethylcarbamoyl)-4-methylpyridazin-3-yl)-3,6-dihydropyridine-1(2H)-carboxylate (1.3 g). Int. 1Z60 (1.3 g) was stirred ON in 2.8M HCl in dioxane (10 mL). The residue after concentration was triturated in EtOAc to give Int. 1Z61 N,N,5-trimethyl-6-(1,2,3,6-tetrahydropyridin-4-yl) pyridazine-3-carboxamide (0.9, HCl salt). STAB (0.30 g) and Ints. 1AB8/1Z61 (0.1 g/0.1 g) were stirred ON in DCE/Et₃N (73:1, 10.1 mL) at 0° C. to RT. The residue after concentration was dissolved in THF, filtered through silica, and concentrated to give Int. 1Z57 6-(1-((4-bromo-1-methyl-1H-pyrrolo[2,3-b]pyridin-2-yl)methyl)-1,2,3,6-tetrahydropyridin-4-yl)-N,N,5-trimethylpyridazine-3-carboxamide (0.19 g).

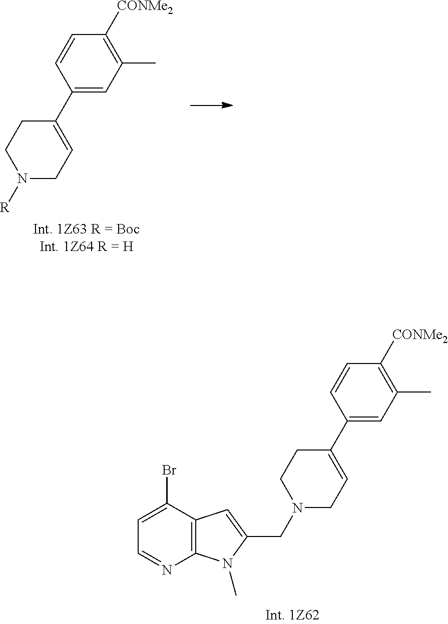

PdDPPFCl₂-DCM (1.1 g), 4-bromo-N,N,2-trimethylbenzamide (3.3 g), K₂CO₃ (5.7 g), and tert-butyl 4-(4,4,5,5-tetramethyl-1,3,2-dioxaborolan-2-yl)-3,6-dihydropyridine-1 (2H)-carboxylate (5.9 g) were refluxed ON in dioxane/H₂O (15:4, 190 mL) under an inert atmosphere. The OL (aq. NaHCO/EtOAc) was dried and concentrated to give Int. 1Z63 tert-butyl 4-(4-(dimethylcarbamoyl)-3-methyl-phenyl)-3,6-dihydropyridine-1(2H)-carboxylate (4.1 g). Int. 1Z63 (4.1 g) was stirred ON in 2.8M HCl in dioxane (30 mL). The residue after concentration was triturated in EtOAc to give Int. 1Z64 N,N,2-trimethyl-4-(1,2,3,6-tetrahydropyridin-4-yl)benzamide (2.3 g, HCl salt). STAB (0.30 g) and Ints. 1AB8/1Z64 (0.1 g/0.1 g) were stirred ON in DCE/Et₃N (73:1, 10.1 mL) at 0° C. to RT. The residue after concentration was dissolved in THF, filtered through silica, and concentrated to give Int. 1Z62 4-(1-((4-bromo-1-methyl-1H-pyrrolo[2,3-b]pyridin-2-yl)methyl)-1,2,3,6-tetrahydropyridin-4-yl)-N,N,2-trimethylbenzamide (0.19 g).

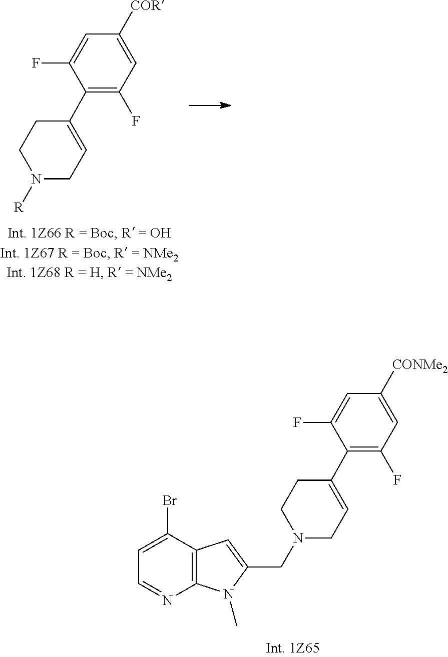

LiOH H₂O (9.5 g) and Int. 3C₁₈ (16 g) were stirred 16 h in THF/MeOH/H₂O (2:1:1, 300 mL) at 0° C. to RT. The residue after concentration was stirred in aq. citric acid to precipitate Int. 1Z66 4-(1-(tert-butoxy-carbonyl)-1,2,3,6-tetrahydropyridin-4-yl)-3,5-difluorobenzoic acid (10 g). Int. 1Z66 (5 g), HATU (8.4 g), and dimethylamine hydrochloride (1.8 g) were stirred 16 h in DMF (40 mL) and DIPEA (13 mL) at 0° C. to RT. The OL (water/EtOAc) was dried, concentrated, and purified by FC (PE/EtOAc 2:1) to give Int. 1Z67 tert-butyl 4-(4-(dimethylcarbamoyl)-2,6-difluorophenyl)-3,6-dihydropyridine-1(2H)-carboxylate (3.7 g). Int. 1Z67 (3.7 g) was stirred 16 h in 2M HCl in dioxane (60 mL) at 0° C. to RT. The mixture was concentrated to give Int. 1Z68 3,5-difluoro-N,N-dimethyl-4-(1,2,3,6-tetrahydropyridin-4-yl)benzamide (2.9 g, HCl salt). Ints. 1J1/1Z68 (0.35 g/0.49 g), KI (22 mg), and K₂CO₃ (0.56 g) were stirred 16 h in ACN (20 mL) at 0° C. to RT. The OL (water/EtOAc) was dried, concentrated, and purified by FC (PE/EtOAc 3:7) to give Int. 1Z65 4-(1-((4-bromo-1-methyl-1H-pyrrolo[2,3-b]pyridin-2-yl)methyl)-1,2,3,6-tetrahydropyridin-4-yl)-3,5-difluoro-N,N-dimethylbenzamide (0.35 g).

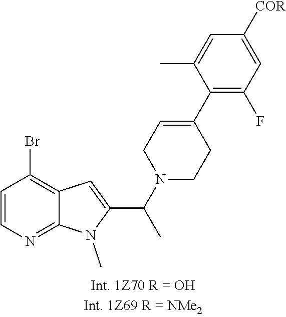

LiOH H₂O (33 g) and Int. 1AF87 (76 g) were stirred 16 h in THE/MeOH/H₂O (2:1:1, 1.5 L) at 0° C. to RT. The residue after concentration was stirred in aq. citric acid at 0° C. to precipitate Int. 1Z70 4-(1-(1-(4-bromo-1-methyl-1H-pyrrolo[2,3-b]pyridin-2-yl)ethyl)-1,2,3,6-tetrahydropyridin-4-yl)-3-fluoro-5-methylbenzoic acid (68 g). Int. 1Z70 (70 g), dimethylamine hydrochloride (36 g), and HATU (84 g) were stirred 12 h in DMF (300 mL) and DIPEA (180 mL) at 0° C. to RT. The mixture was diluted with water to precipitate a solid that was dried to give Int. 1Z69 4-(1-(1-(4-bromo-1-methyl-1H-pyrrolo-[2,3-b]pyridin-2-yl)ethyl)-1,2,3,6-tetrahydropyridin-4-yl)-3-fluoro-N,N,5-trimethylbenzamide (63 g).

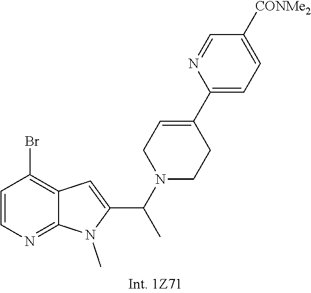

Ints. 1AF88/1S4 (1.5 g/1.5 g), KI (91 mg), and Cs₂CO₃ (8.9 g) were stirred 16 h in ACN (15 mL) at 0° C. to RT. The OL (water/EtOAc) was dried, concentrated, and purified by FC (DCM/MeOH 19:1) to give Int. 1Z71 1′-(1-(4-bromo-1-methyl-1H-pyrrolo[2,3-b]pyridin-2-yl)ethyl)-N,N-dimethyl-1′,2′,3′,6′-tetrahydro-[2,4′-bipyridine]-5-carboxamide (0.50 g).

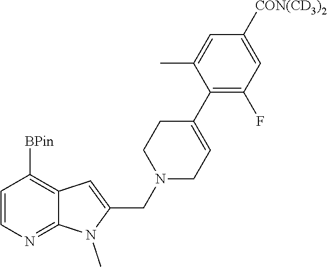

3-fluoro-5-methyl-N,N-bis(methyl-d₃)-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide Int. 1AE9 (0.45 g), bis (pinacolato)diboron (0.35 g), PdDPPFCl₂-DCM (75 mg), and KOAc (0.27 g) were stirred 1 h in dioxane (5 mL) at 110° C. under MW conditions. The mixture was filtered through celite and concentrated to give Int. 1Z73 3-fluoro-5-methyl-N,N-bis(methyl-d₃)-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.40 g).

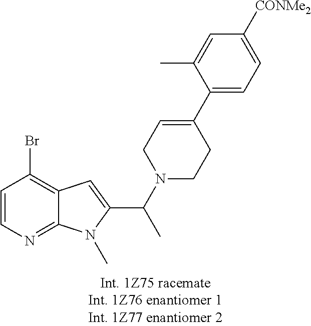

Int. 1AE38 (3.2 g) and Cs₂CO₃ (18 g) were stirred 0.3 h in ACN (20 mL). Int. 1AF88 (3 g) and KI (0.91 g) were added and stirring continued 12 h at 0° C. to RT. The OL (water/EtOAc) was dried, concentrated, and purified by FC (hexane/EtOAc 1:9) to give Int. 1Z75 4-(1-(1-(4-bromo-1-methyl-1H-pyrrolo[2,3-b]pyridin-2-yl)ethyl)-1,2,3,6-tetrahydropyridin-4-yl)-N,N,3-trimethylbenzamide (0.90 g). Int. 1Z75 (0.90 g) was resolved by SFC on a SFC-150-022 instrument fitted with a Chiral ART Amylose-C NEO 250×30 mm 5 μm column operated at 30° C. and an eluent of 80% CO₂ and 20% MeOH at a (100 g/min) and a back pressure of 100 bar to give Int. 1Z76 (0.15 g, first peak) and Int. 1Z77 (0.15 g, second peak) (S)-4-(1-(1-(4-bromo-1-methyl-1H-pyrrolo[2,3-b]pyridin-2-yl)ethyl)-1,2,3,6-tetrahydropyridin-4-yl)-N,N,3-trimethylbenzamide and (R)-4-(1-(1-(4-bromo-1-methyl-1H-pyrrolo[2,3-b]pyridin-2-yl)ethyl)-1,2,3,6-tetrahydropyridin-4-yl)-N,N,3-trimethylbenzamide. The absolute configurations of these compounds were not determined.

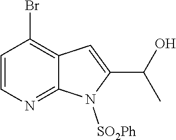

3M CH₃MgCl in THF (32 mL) was added slowly to a solution of Int. 1A6 (11.5 g) in THF (310 mL) at −78° C. The mixture was stirred 0.5 h at −78° C. to RT. The OL (aq. NH₄Cl/EtOAc) was dried, and concentrated to give Int. 1Z79 1-(4-bromo-1-(phenylsulfonyl)-1H-pyrrolo[2,3-b]pyridin-2-yl) ethan-1-ol (12 g).

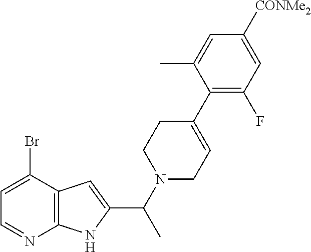

SOCl₂ (0.18 mL) was added to a solution of Int. 1Z79 (0.1 g) in toluene (3 mL). The mixture was stirred 0.5 h and concentrated. Int. 1AE12′ (90 mg), KI (20 mg), and Cs₂CO₃ (0.40 g) were stirred ON in a solution of the residue in ACN (5 mL). The mixture was HPLC-purified to give Int. 1Z80 4-(1-(1-(4-bromo-1H-pyrrolo[2,3-b]pyridin-2-yl)ethyl)-1,2,3,6-tetrahydropyridin-4-yl)-3-fluoro-N,N,5-trimethylbenzamide (13 mg).

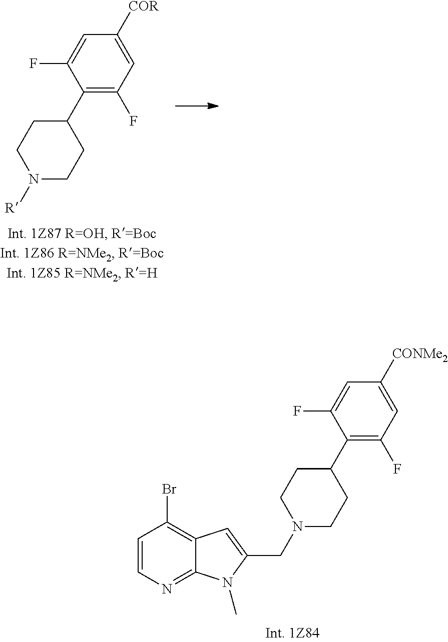

LiOH H₂O (9.8 g) and Int. 3C₁₇ (16.5 g) were stirred 16 h in THF/MeOH/H₂O (2:1:1, 600 mL) at 0° C. to RT. The residue after concentration was stirred in aq. citric acid, filtered, and dried to give Int. 1Z87 4-(1-(tert-butoxycarbonyl) piperidin-4-yl)-3,5-difluorobenzoic acid (11.5 g). HATU (8.3 g), Int. 1Z87 (5 g), and dimethylamine hydrochloride (1.8 g) were stirred 16 h in DMF (30 mL) and DIPEA (13 mL) 0° C. to RT. The OL (water/EtOAc) was dried, concentrated, and purified by FC (PE/EtOAc 2:1) to give Int. 1Z86 tert-butyl 4-(4-(dimethylcarbamoyl)-2,6-difluorophenyl) piperidine-1-carboxylate (3.2 g). Int. 1Z86 (3.2 g) was stirred 16 h in 2M HCl in dioxane (60 mL) at 0° C. to RT. The mixture was concentrated to give Int. 1Z85 3,5-difluoro-N,N-dimethyl-4-(piperidin-4-yl)benzamide (2.3 g, HCl salt). Ints. 1J1/1Z85 (0.35 g/0.36 g) and K₂CO₃ (0.93 g) were stirred 16 h in ACN (10 mL) at 0° C. to RT. The mixture was filtered, concentrated, and purified by FC (PE/EtOAc 1:4) to give Int. 1Z84 4-(1-((4-bromo-1-methyl-1H-pyrrolo[2,3-b]pyridin-2-yl)methyl) piperidin-4-yl)-3,5-difluoro-N,N-dimethyl-benzamide (0.40 g).

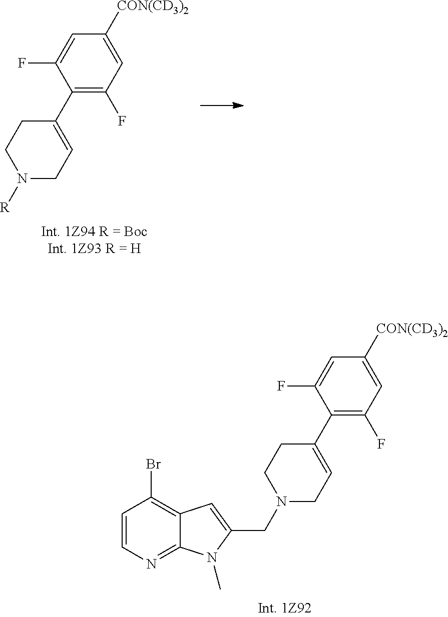

Int. 1Z66 (5 g), bis(methyl-d₃) amine hydrochloride (1.5 g), and HATU (8.4 g) were stirred 16 h in DMF (40 mL) and DIPEA (13 mL) at 0° C. to RT. The OL (water/EtOAc) was dried, concentrated, and purified by FC (PE/EtOAc 2:1) to give Int. 1Z94 tert-butyl 4-(4-(bis(methyl-d₃)carbamoyl)-2,6-difluoro-phenyl)-3,6-dihydropyridine-1(2H)-carboxylate (3.5 g). Int. 1Z94 (3.6 g) was stirred 16 h in 2M HCl in dioxane (60 mL) at 0° C. to RT. The mixture was concentrated to give Int. 1Z93 3,5-difluoro-N,N-bis-(methyl-d₃)-4-(1,2,3,6-tetrahydropyridin-4-yl)benzamide (2.7 g, HCl salt). Ints. 1J1/1Z93 (0.40 g/0.57 g), KI (77 mg), and K₂CO₃ (0.64 g) were stirred 16 h in ACN (20 mL) at 0° C. to RT. The OL (water/EtOAc) was dried, concentrated and, purified by FC (PE/EtOAc 3:7) to give Int. 1Z92 4-(1-((4-bromo-1-methyl-1H-pyrrolo[2,3-b]pyridin-2-yl)methyl)-1,2,3,6-tetrahydropyridin-4-yl)-3,5-difluoro-N,N-bis(methyl-d₃) benzamide (0.41 g).

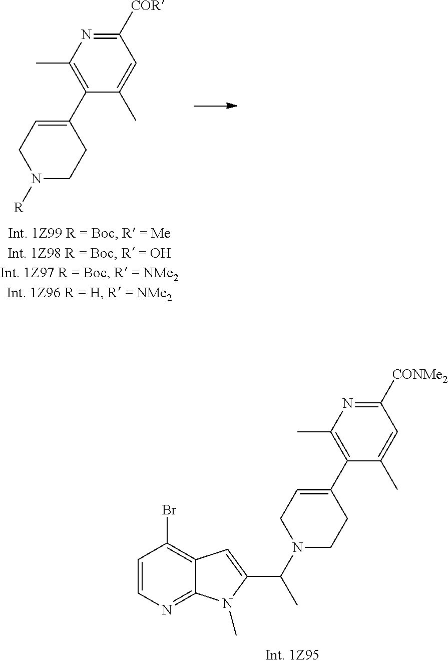

PdDPPFCl₂-DCM (0.2 g), methyl 5-bromo-4,6-dimethylpicolinate (1.1 g), K₂CO₃ (2 g), and tert-butyl 4-(4,4,5,5-tetramethyl-1,3,2-dioxaborolan-2-yl)-3,6-dihydropyridine-1(2H)-carboxylate (1.5 g) were stirred 12 h in dioxane (8:1, 45 mL) at 80° C. under an inert atmosphere. The mixture was filtered and concentrated to give Int. 1Z99 1′-(tert-butoxycarbonyl)-2,4-dimethyl-1′,2′,3′,6′-tetrahydro-[3,4′-bipyridine]-6-carboxylic acid (1.4 g). LiOH H₂O (0.3 g) and Int. 1Z99 (1.4 g) were stirred 12 h in MeOH/H₂O (1:1, 60 mL). The mixture was concentrated to give Int. 1Z98 1′-(tert-butoxycarbonyl)-2,4-dimethyl-1′,2′,3′,6′-tetrahydro-[3,4′-bipyridine]-6-carboxylic acid (1.3 g, Li salt). Int. 1Z98 (1.4 g), dimethylamine hydrochloride (0.4 g) and HATU (1.7 g) were stirred 12 h in DMF (50 mL) and Et₃N (2.8 mL). The mixture was concentrated. The OL (water/EtOAc) was concentrated to give Int. 1Z97 tert-butyl 6-(dimethylcarbamoyl)-2,4-dimethyl-3′, 6′-dihydro-[3,4′-bipyridine]-1′ (2′H)-carboxylate (0.95 g). Int. 1Z97 (0.95 g) was stirred ON in MeOH/2.8M HCl in dioxane (1:1, 40 mL). The mixture was concentrated to give Int. 1Z96 N,N,2,4-tetramethyl-1′,2′,3′,6′-tetrahydro-[3,4′-bipyridine]-6-carboxamide (0.7 g, HCl salt). Int. 1AF89 (0.3 g) was stirred 0.5 h in toluene (20 mL) and SOCl₂ (0.61 mL) at 80° C. The mixture was concentrated. The residue, Int. 1Z96 (0.5 g), and Cs₂CO₃ (0.3 g) were stirred 12 h in DMF (10 mL). The OL (water/EtOAc) was concentrated and HPLC-purified to give Int. 1Z95 1′-(1-(4-bromo-1-methyl-1H-pyrrolo[2,3-b]pyridin-2-yl)ethyl)-N,N,2,4-tetramethyl-1′,2′,3′,6′-tetrahydro-[3,4′-bipyridine]-6-carboxamide (0.5 g).

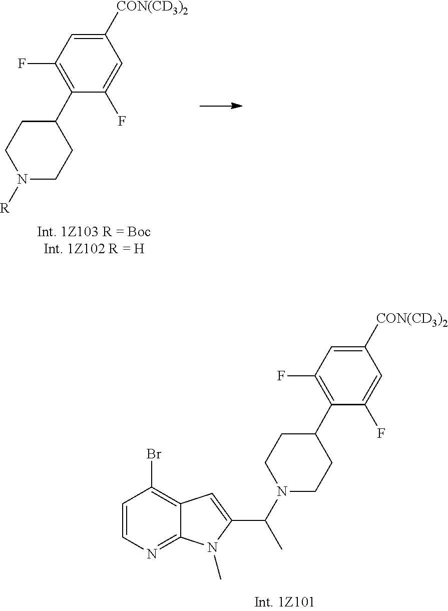

Int. 1Z87 (2 g), bis(methyl-d₃) amine hydrochloride (0.62 g), and HATU (3.3 g) were stirred 16 h in DMF (30 mL) and DIPEA (5.1 mL) at 0° C. to RT. The OL (water/EtOAc) was dried, concentrated, and purified by FC (PE/EtOAc 2:1) to give Int. 1Z103 tert-butyl 4-(4-(bis(methyl-d₃)carbamoyl)-2,6-difluorophenyl)-piperidine-1-carboxylate (1.4 g). Int. 1Z103 (1.4 g) was stirred 16 h in 2.2M HCl in dioxane (18 mL) at 0° C. to RT. The mixture was concentrated to give Int. 1Z102 3,5-difluoro-N,N-bis(methyl-d₃)-4-(piperidin-4-yl)benzamide (1.1 g, HCl salt). Ints. 1J1/1Z102 (0.50 g/0.60 g), KI (0.16 g), and K₂CO₃ (1.3 g) were stirred 16 h in ACN (20 mL) at 0° C. to RT. The OL (water/EtOAc) was concentrated and purified by FC (hexane/EtOAc 1:4) to give Int. 1Z101 4-(1-((4-bromo-1-methyl-1H-pyrrolo[2,3-b]pyridin-2-yl)methyl) piperidin-4-yl)-3,5-difluoro-N,N-bis(methyl-d₃) benzamide (0.45 g).

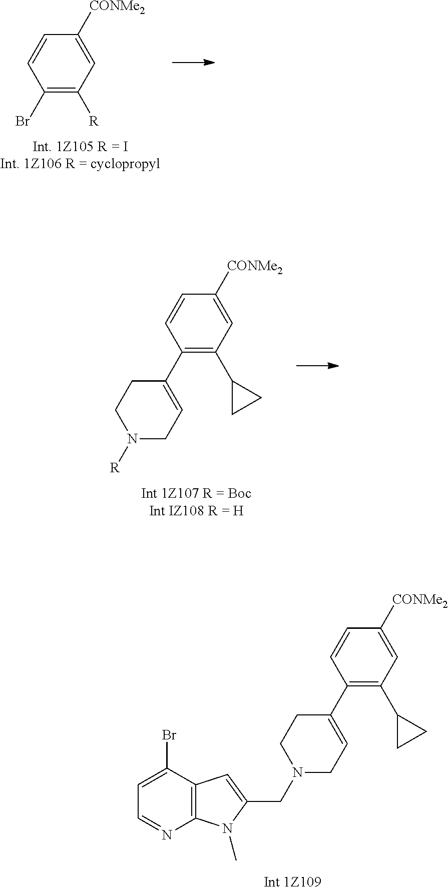

NaIO₄ (2.6 g) was added portion-wise to a mixture of I₂ (8.9 g) in sulfuric acid (100 mL). The mixture was stirred 0.5 h before 4-bromo-N,N-dimethylbenzamide (17 g) was added and stirring continued for 18 h. The mixture was diluted with water to precipitate a solid that was dried and crystallized from CCI to give Int. 1Z105 4-bromo-3-iodo-N,N-dimethylbenzamide (17 g). PdDPPFCl₂-DCM (2.0 g), Int. 1Z105 (17 g), cyclopropylboronic acid (5.0 g), and K₂CO₃ (20 g) were refluxed ON in dioxane/H₂O (3:1, 200 mL) under an inert atmosphere. The mixture was concentrated and purified by FC (CHCl₃ to CHCl₃/ACN 1:1) to give Int. 1Z106 4-bromo-3-cyclopropyl-N,N-dimethylbenzamide (12 g). PdDPPFCl₂-DCM (1.9 g), Int. 1Z106 (12 g), tert-butyl 4-(4,4,5,5-tetramethyl-1,3,2-dioxaborolan-2-yl)-3,6-dihydropyridine-1(2H)-carboxylate (21 g), and K₂CO₃ (19 g) were refluxed ON in dioxane/H₂O (6:1, 140 mL) under an inert atmosphere. The mixture was concentrated and purified by FC (CHCl₃ to CHCl₃/ACN 1:1) to give Int. 1Z107 tert-butyl 4-(2-cyclopropyl-4-(dimethylcarbamoyl)phenyl)-3,6-dihydropyridine-1(2H)-carboxylate (4.1 g). Int. 1Z107 (4.1 g) was stirred in ON 4M HCl in dioxane (50 mL). The mixture was concentrated and triturated in EtOAc to give Int. 1Z108 3-cyclopropyl-N,N-dimethyl-4-(1,2,3,6-tetrahydropyridin-4-yl)benzamide (0.10 g, HCl salt). STAB (0.30 g) and Ints. 1AB8/1Z108 (0.1 g/0.1 g) were stirred ON in DCE/Et₃N (73:1, 10.1 mL) at 0° C. to RT. The mixture was concentrated, dissolved in THF, filtered through silica, and concentrated to give Int. 1Z109 4-(1-((4-bromo-1-methyl-1H-pyrrolo[2,3-b]pyridin-2-yl)methyl)-1,2,3,6-tetrahydropyridin-4-yl)-3-cyclopropyl-N,N-dimethylbenzamide (0.19 g).

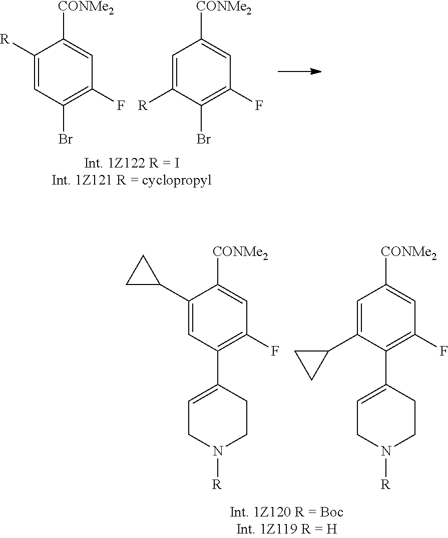

NaIO₄ (2.3 g) was added to a mixture of I₂ (8.0 g) in sulfuric acid (100 mL) portion-wise and stirred 0.5 h. 4-Bromo-3-fluoro-N,N-dimethylbenzamide (17 g) was added and stirring continued 18 h. The reaction mixture was diluted with water to precipitate a solid that was dried and precipitated from CCl₄ to give Int. 1Z122, a mixture of 4-bromo-5-fluoro-2-iodo-N,N-dimethylbenzamide and 4-bromo-3-fluoro-5-iodo-N,N-dimethylbenzamide mixture (13 g). PdDPPFCl₂-DCM (1.4 g), Int. 1Z122 (13 g), cyclopropylboronic acid (3.6 g), and K₂CO₃ (14 g) were refluxed ON in dioxane/H₂O (3:1, 200 mL) under an inert atmosphere. The mixture was concentrated and purified by FC (CHCl₃ to CHCl₃/ACN 1:1) to give Int. 1Z121, a mixture of 4-bromo-2-cyclopropyl-5-fluoro-N,N-dimethylbenzamide and 4-bromo-3-cyclopropyl-5-fluoro-N,N-dimethylbenzamide mixture (7 g). PdDPPFCl₂-DCM (1.0 g), Int. 1Z121 (7 g), tert-butyl 4-(4,4,5,5-tetramethyl-1,3,2-dioxaborolan-2-yl)-3,6-dihydropyridine-1(2H)-carboxylate (11 g), and K₂CO₃ (10 g) were refluxed ON in dioxane/H₂O (6:1, 140 mL) under an inert atmosphere. The mixture was concentrated and purified by FC (CHCl₃ to CHCl₃/ACN 1:1) to give Int. 1Z120, a mixture of tert-butyl 4-(5-cyclopropyl-4-(dimethylcarbamoyl)-2-fluorophenyl)-3,6-dihydropyridine-1(2H)-carboxylate and tert-butyl 4-(2-cyclopropyl-4-(dimethylcarbamoyl)-6-fluoro-phenyl)-3,6-dihydropyridine-1(2H)-carboxylate mixture (2.6 g). Int. 1Z120 (2.6 g) was stirred in ON 4M HCl in dioxane (50 mL). The mixture was concentrated and triturated in EtOAc to give Int. 1Z119 2-cyclopropyl-5-fluoro-N,N-dimethyl-4-(1,2,3,6-tetrahydropyridin-4-yl)benzamide and 3-cyclopropyl-5-fluoro-N,N-dimethyl-4-(1,2,3,6-tetrahydropyridin-4-yl)benzamide mixture (2.0 g, HCl salt).

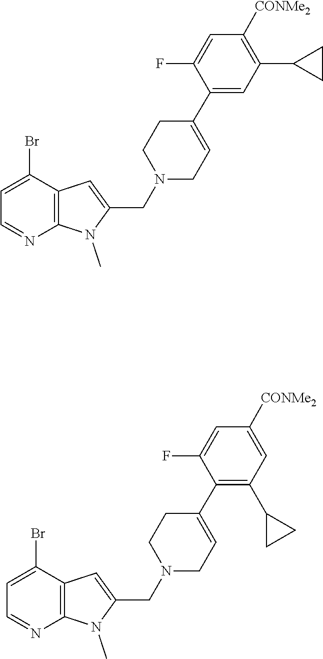

STAB (0.30 g) and Ints. 1AB8/1Z119 (0.1 g/0.1 g) were stirred ON in DCE/Et₃N (73:1, 10.1 mL) at 0° C. to RT. The residue after concentration dissolved in THF, filtered through silica, and concentrated to give a mixture of Ints. 1Z123/1Z124 4-(1-((4-bromo-1-methyl-1H-pyrrolo[2,3-b]pyridin-2-yl)methyl)-1,2,3,6-tetrahydropyridin-4-yl)-2-cyclopropyl-5-fluoro-N,N-dimethylbenzamide and 4-(1-((4-bromo-1-methyl-1H-pyrrolo[2,3-b]pyridin-2-yl)methyl)-1,2,3,6-tetrahydropyridin-4-yl)-3-cyclopropyl-5-fluoro-N,N-dimethylbenzamide (0.17 g).

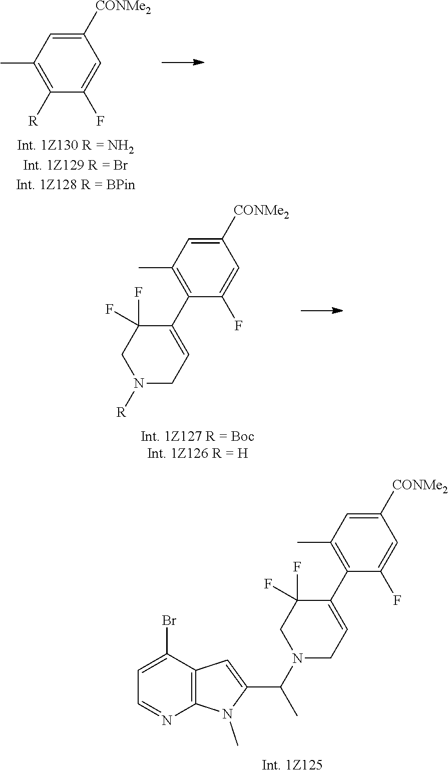

N-Iodosuccinimide (196 g) and 2-fluoro-6-methylaniline (100 g) were stirred 2 h in ACN (2.0 L) at 0° C. to RT. The OL (aq. Na₂S₂O₃/EtOAc) was dried, and concentrated to give Int. 1Z131 2-fluoro-4-iodo-6-methylaniline (180 g). Int. 1Z131 (30 g), dimethylamine hydrochloride (24 g), and PdDPPFCl₂-DCM (4.9 g) were stirred 16 h in dioxane/Et₃N (14:3, 364 mL) at 85° C. under 200 psi of CO. The mixture was filtered and concentrated. The OL (1M HCl/EtOAc) was neutralized to PH ˜8 with Na₂CO₃. The OL (water/EtOAc) was dried, and concentrated to give Int. 1Z130 4-amino-3-fluoro-N,N,5-trimethylbenzamide (17 g). CuBr₂ (85 g) and 1Z130 (15 g) were stirred 16 h in ACN (300 mL) and ^{t}BuONO (14 mL) at 0-85° C. The OL (aq. NH₃/PE) was dried, filtered and concentrated to give Int. 1Z129 4-bromo-3-fluoro-N,N,5-trimethylbenzamide (14 g). Int. 1Z129 (1 g), PdDPPFCl₂-DCM (0.31 g), bis (pinacolato)diboron (2.0 g), and KOAc (1.1 g) were stirred 1 h in dioxane (10 mL) at 110° C. under MW conditions. The mixture was filtered through celite, concentrated, and washed with pentane to give Int. 1Z128 3-fluoro-N,N,5-trimethyl-4-(4,4,5,5-tetramethyl-1,3,2-dioxaborolan-2-yl)benzamide (0.80 g). PdDPPFCl₂-DCM (0.11 g), Ints. 3E284/1Z128 (0.50 g/0.77 g), and Na₂CO₃ (0.43 g) were stirred 1.5 h in dioxane/H₂O (4:1, 10 mL) at 110° C. under MW conditions. The mixture was filtered. The OL (water/EtOAc) was concentrated and purified by FC (hexane/EtOAc 3:7) to give Int. 1Z127 tert-butyl 4-(4-(dimethylcarbamoyl)-2-fluoro-6-methylphenyl)-3,3-difluoro-3,6-dihydro-pyridine-1(2H)-carboxylate (65 mg). Int. 1Z127 (0.50 g) was stirred 16 h in DCM/4M HCl in dioxane (2:1, 6 mL) at 0° C. to RT. The mixture was concentrated to give Int. 1Z126 4-(3,3-difluoro-1,2,3,6-tetrahydro-pyridin-4-yl)-3-fluoro-N,N,5-trimethylbenzamide (0.40 g, HCl salt). Ints. 1AF88/1Z126 (0.40 g/0.59 g), KI (0.17 g), and Cs₂CO₃ (3.3 g) were stirred 12 h in ACN (10 mL) at 0° C. to RT. The mixture was filtered, concentrated, and purified by FC (hexane/EtOAc 4:1) to give Int. 1Z125 4-(1-(1-(4-bromo-1-methyl-1H-pyrrolo[2,3-b]-pyridin-2-yl)ethyl)-3,3-difluoro-1,2,3,6-tetrahydropyridin-4-yl)-3-fluoro-N,N,5-trimethylbenzamide (0.13 g).

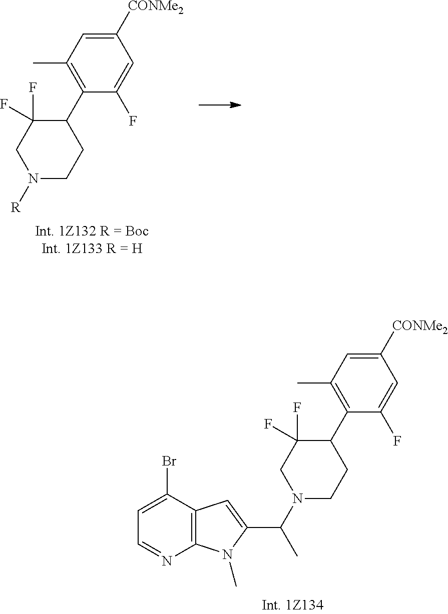

Int. 1Z127 (0.40 g) and PtO₂ (0.80 g) were hydrogenated 75 h at 70 psi in THF/EtOH/AcOH (8:8:1, 17 mL). The mixture was filtered and concentrated to give Int. 1Z132 tert-butyl 4-(4-(dimethyl-carbamoyl)-2-fluoro-6-methylphenyl)-3,3-difluoropiperidine-1-carboxylate (0.40 g). Int. 1Z133 4-(3,3-difluoropiperidin-4-yl)-3-fluoro-N,N,5-trimethylbenzamide (0.20 g, HCl salt) was prepared similarly to Int. 1Z126 from Int. 1Z132 (0.25 g). Ints. 1AF88/1Z133 (0.15 g/0.18 g), KI (64 mg), and Cs₂CO₃ (0.12 g) were stirred 16 h in ACN (10 mL) at 0° C. to RT. The OL (water/EtOAc) was dried, concentrated, and purified by FC (PE/EtOAc 1:1 to 1:2) to give Int. 1Z134 4-(1-(1-(4-bromo-1-methyl-1H-pyrrolo[2,3-b]pyridin-2-yl)ethyl)-3,3-difluoropiperidin-4-yl)-3-fluoro-N,N,5-trimethyl-benzamide (0.13 g).

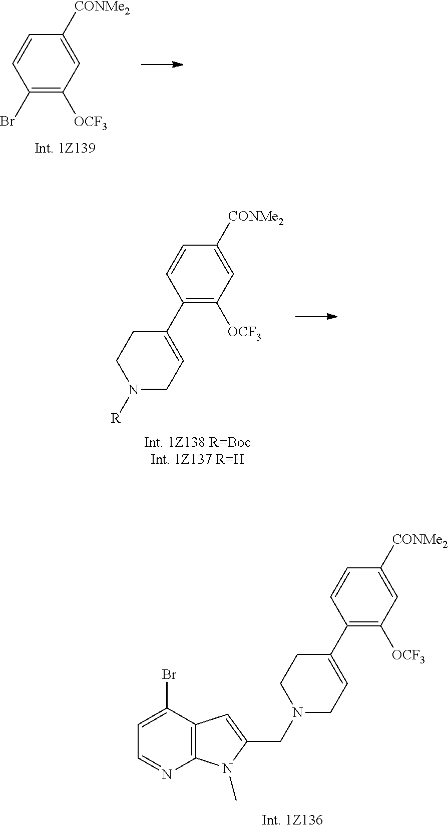

Di(1H-imidazol-1-yl) methanone (1.0 g) and 4-bromo-3-(trifluoromethoxy)benzoic acid (1.8 g) were refluxed 0.3 h in dioxane (50 mL). Dimethylamine hydrochloride (0.51 g) and Et₃N (5.2 mL) was added and stirring continued 10 h. The mixture was concentrated. The OL (aq. NaHSO₄/CHCl₃) was concentrated to give Int. 1Z139 4-bromo-N,N-dimethyl-3-(trifluoro (oxo)-26-methyl)benzamide (1.5 g). Int. 1Z139 (1.5 g), tert-butyl 4-(4,4,5,5-tetramethyl-1,3,2-dioxaborolan-2-yl)-3,6-dihydropyridine-1(2H)-carboxylate (1.6 g), K₂CO₃ (2.0 g), and PdDPPFCl₂-DCM (0.20 g) were stirred ON in dioxane/H₂O (4:1, 20 mL) at 100° C. under an inert atmosphere. The mixture was concentrated and purified by FC to give Int. 1Z138 tert-butyl 4-(4-(dimethylcarbamoyl)-2-(trifluoromethoxy)phenyl)-3,6-dihydro-pyridine-1(2H)-carboxylate (0.78 g). Int. 1Z138 (0.78 g) was stirred ON in 2.8M HCl in dioxane (10 mL). The mixture was concentrated to give Int. 1Z137 N,N-dimethyl-4-(1,2,3,6-tetrahydropyridin-4-yl)-3-(trifluoromethoxy)benzamide (0.43 g, HCl salt). Ints. 1J1/1Z137 (0.36 g/0.43 g) and K₂CO₃ (0.57 g) were stirred 11 h in DMF (10 mL). The mixture was diluted with water to precipitate Int. 1Z136 4-(1-((4-bromo-1-methyl-1H-pyrrolo[2,3-b]pyridin-2-yl)methyl)-1,2,3,6-tetrahydropyridin-4-yl)-N,N-dimethyl-3-(trifluoromethoxy)benzamide (0.14 g).

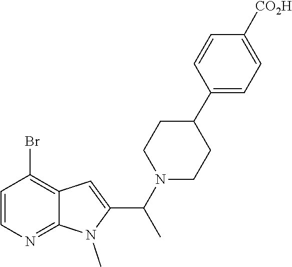 [SMILES: CC(c1cc2c(Br)ccnc2n1C)N1CCC(c2ccc(C(=O)O)cc2)CC1]

Int. C3-4-(1-(1-(4-bromo-1-methyl-1H-pyrrolo[2,3-b]pyridin-2-yl)ethyl) piperidin-4-yl)benzoic acid (0.11 g) was prepared similarly to Int. 2C9 from Int. 3E37 (0.12 g).

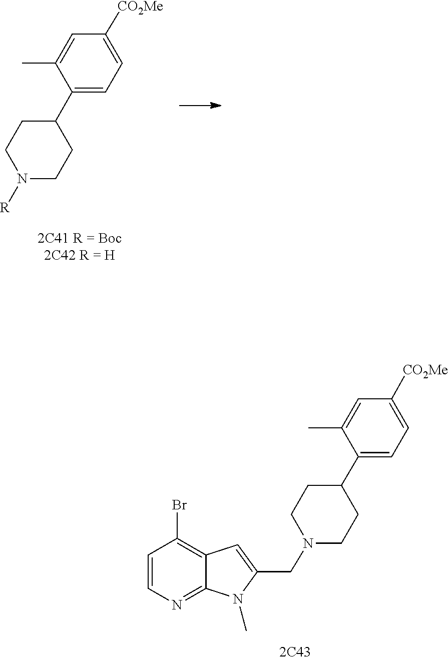

Methyl 4-bromo-3-methylbenzoate (2.5 g), tert-butyl 4-(4,4,5,5-tetramethyl-1,3,2-dioxaborolan-2-yl)-3,6-dihydropyridine-1(2H)-carboxylate (4.0 g), NaHCO₃ (2.8 g), and PdDPPFCl₂-DCM (0.45 g) were degassed in dioxane/water (10:1, 30 mL) and stirred ON at 100° C. The mixture was diluted with EtOAc (100 mL), filtered, concentrated, and purified by FC (hexane/EtOAc 9:1 to 85:15) to give Int. 2C41 tert-butyl 4-(4-methoxy-carbonyl-2-methyl-phenyl)-3,6-dihydro-2H-pyridine-1-carboxylate (3.2 g). Int. 2C41 (0.25 g) was stirred ON in DCM/4M HCl in dioxane (₄:1, 10 mL) and concentrated. The residue was triturated in pentane to give Int. 2C42 methyl 3-methyl-4-(1,2,3,6-tetra-hydropyridin-4-yl)benzoate (0.19 g, HCl salt). Ints. 1AB8/2C42 (1.2 g/1.2 g, HCl salt) were stirred 4 h in DCE/DIPEA (2:2:1, 7.3 mL) at 0° C. to RT. STAB (1.9 g) was added and stirring continued ON. The OL (EtOAc/water) was dried, concentrated, and purified by FC (hexane/EtOAc 7:3) to give Int. 2C43 methyl 4-(1-((4-bromo-1-methyl-1H-pyrrolo[2,3-b]pyridin-2-yl)methyl)-1,2,3,6-tetrahydropyridin-4-yl)-3-methyl-benzoate (1.0 g).

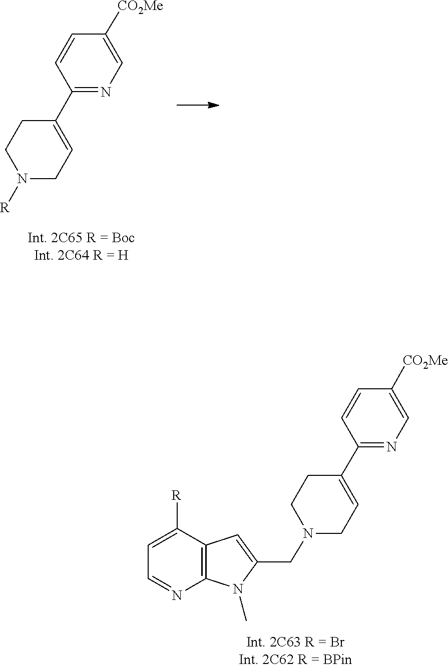

Methyl 6-bromonicotinate (5.0 g), tert-butyl 4-(4,4,5,5-tetramethyl-1,3,2-dioxaborolan-2-yl)-3,6-dihydropyridine-1(2H)-carboxylate (7.8 g), K₂CO₃ (3.0 g) and PdDPPFCl₂-DCM (1.9 g) were stirred in dioxane/H₂O (4:1, 100 mL) ON at 100° C. under an inert atmosphere. The OL (water/EtOAc) was dried, concentrated and purified by FC (hexane/MTBE 1:0 to 0:1) to give Int. 2C65 1′-(tert-butyl) 5-methyl 3′, 6′-dihydro-[2,4′-bipyridine]-1′, 5 (2′H)-dicarboxylate (5.9 g). Int. 2C65 (2.0 g) was stirred 16 h in 2M HCl in dioxane (40 mL), concentrated, and washed with Et₂O to give Int. 2C64 methyl 1′,2′,3′,6′-tetrahydro-[2,4′-bipyridine]-5-carboxylate (1.8 g, HCl salt). Ints. 1AB8/2C64 (616 mg/750 mg) and 4 Å MS were stirred ON in DCE/Et₃N (9:1, 11.1 mL). (AcO)₃BHNa (1.6 g) was added and stirring continued for 3 h before it was concentrated and HPLC-purified to give Int. 2C63 methyl 1′-((4-bromo-1-methyl-1H-pyrrolo[2,3-b]pyridin-2-yl)methyl)-1′,2′,3′,6′-tetrahydro-[2,4′-bipyridine]-5-carboxylate (0.57 g). Int. 2C63 (0.29 g), bis (pinacolato)diboron (0.18 g), KOAc (0.19 g), and PdDPPFCl₂-DCM (53 mg) were stirred ON in dioxane (10 mL) at 80° C. under an inert atmosphere. The residue after concentration was dissolved in THF, filtered through silica, and concentrated to give Int. 2C62 methyl 1′-((1-methyl-4-(4,4,5,5-tetramethyl-1,3,2-dioxaborolan-2-yl)-1H-pyrrolo[2,3-b]pyridin-2-yl)-methyl)-1′,2′,3′,6′-tetrahydro-[2,4′-bipyridine]-5-carboxylate (0.30 g).

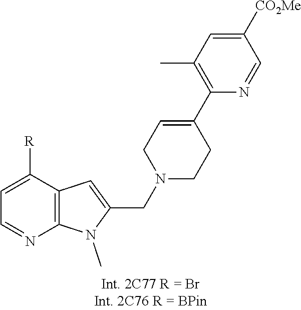

Ints. 1AB8/2C78 (0.50 g/1.0 g) and 4 Å MS were stirred ON in DCE/Et₃N (15:1, 15.9 mL). (AcO)₃BHNa (1.0 g) was added and stirring continued 3 h. The mixture was concentrated and HPLC-purified to give Int. 2C77 methyl 1′-((4-bromo-1-methyl-1H-pyrrolo[2,3-b]pyridin-2-yl)methyl)-3-methyl-1′,2′,3′,6′-tetrahydro-[2,4′-bipyridine]-5-carboxylate (0.61 g). Int. 2C77 (0.30 g), bis (pinacolato)diboron (0.30 mg), KOAc (0.30 mg) and PdDPPFCl₂-DCM (30 mg) were stirred ON in dioxane (3 mL) at 80° C. under an inert atmosphere. The residue after concentration was dissolved in THF, filtered through silica and concentrated to give Int. 2C76 methyl 3-methyl-1′-((1-methyl-4-(4,4,5,5-tetramethyl-1,3,2-dioxaborolan-2-yl)-1H-pyrrolo[2,3-b]pyridin-2-yl)methyl)-1′,2′,3′,6′-tetrahydro-[2,4′-bipyridine]-5-carboxylate (0.30 g).

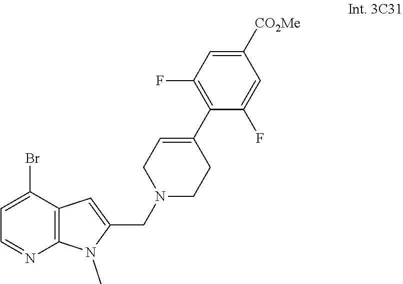 [SMILES: COC(=O)c1cc(F)c(C2=CCN(Cc3cc4c(Br)ccnc4n3C)CC2)c(F)c1]

Ints. 1AB8/3C₂₅ (8.0 g/12.6 g) were stirred 16 h in DCE (80 mL) and DIPEA (35 mL) at 0° C. to RT under an inert atmosphere. STAB (28 g) was added in portions at 0° C. and stirring was continued for 12 h. The OL (water/DCM) was dried, filtered, concentrated, and purified by FC (PE/EtOAc 3:2) to give Int. 3C₃₁ methyl 4-(1-((4-bromo-1-methyl-1H-pyrrolo[2,3-b]pyridin-2-yl)methyl)-1,2,3,6-tetrahydropyridin-4-yl)-3,5-difluorobenzoate (7.3 g).

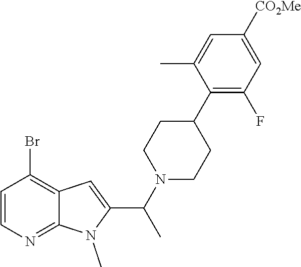 [SMILES: COC(=O)c1cc(C)c(C2CCN(C(C)c3cc4c(Br)ccnc4n3C)CC2)c(F)c1]

Ints. 1AF88/2G97 (2.0 g/2.3 g), KI (0.85 g) and Cs₂CO₃ (9.5 g) were mixed in ACN (20 mL) at 0° C. and stirred for 16 h at RT filtered and concentrated. The residue was purified by FC (hexane/EtOAc 4:1) to give Int. 2G85 methyl 4-(1-(1-(4-bromo-1-methyl-1H-pyrrolo[2,3-b]pyridin-2-yl)ethyl) piperidin-4-yl)-3-fluoro-5-methylbenzoate (2.0 g).

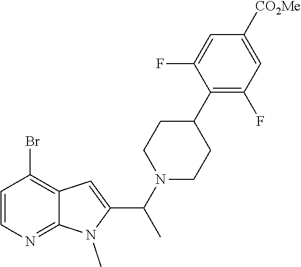 [SMILES: COC(=O)c1cc(F)c(C2CCN(C(C)c3cc4c(Br)ccnc4n3C)CC2)c(F)c1]

Int. 1AF89 (1.5 g) was stirred 0.3 h in toluene/SOCl₂ (7:3, 14.3 mL) at 90° C. The residue after concentration, Cs₂CO₃ (7.7 g), KI (0.49 g), and Int. 3C₁₆ (2.1 g) were stirred 16 h in ACN (10 mL) at 0° C. to RT under an inert atmosphere. The reaction mixture was concentrated and triturated in water to precipitate a solid that was washed with water, dried, and purified by FC (PE/EtOAc 9:1 to 17:3) to give Int. 2G96 methyl 4-(1-(1-(4-bromo-1-methyl-1H-pyrrolo[2,3-b]pyridin-2-yl)ethyl) piperidin-4-yl)-3,5-difluorobenzoate (1.5 g).

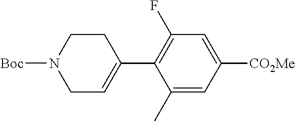 [SMILES: COC(=O)c1cc(C)c(C2=CCN(C(=O)OC(C)(C)C)CC2)c(F)c1]

Int. 1AF90 (15 g), Boc₂O (17 g) and DMAP (0.64 g) were mixed in DCM/Et₃N (4:1, 187 mL) at 0° C. and stirred 16 h and filtered. The OL (water/DCM) was dried, concentrated, and purified by FC (hexane/EtOAc 2:1) to give Int. 2G105 tert-butyl 4-(2-fluoro-4-(methoxycarbonyl)-6-methylphenyl)-3,6-dihydropyridine-1(2H)-carboxylate (14 g).

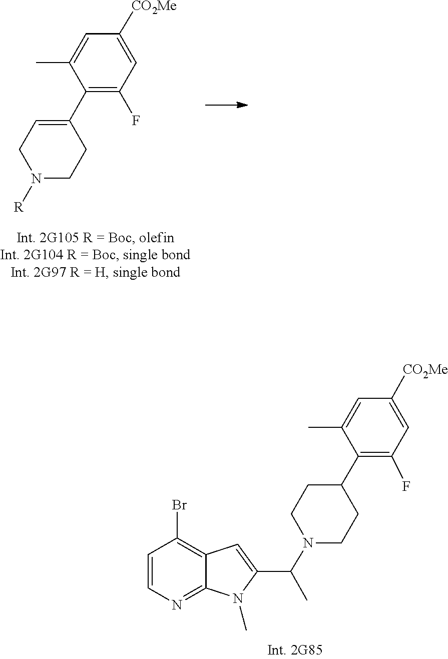

Int. 2G105 (14 g) and Pd/C (7 g) was hydrogenated 16 h in MeOH (140 mL), filtered, concentrated to give Int. 2G104 tert-butyl 4-(2-fluoro-4-(methoxycarbonyl)-6-methylphenyl) piperidine-1-carboxylate (13.2 g). Int. 2G104 (13.2 g) was stirred 16 h in DCM/4M HCl in dioxane (2:5, 105 mL) at 0° C. to RT. The mixture was concentrated and washed with pentane to give Int. 2G97 methyl 3-fluoro-5-methyl-4-(piperidin-4-yl)benzoate (9.0 g, HCl salt). Ints. 1AF88/2G97 (2.0 g/2.3 g), KI (0.85 g), and Cs₂CO₃ (9.5 g) were stirred 16 h in ACN (20 mL) at 0° C. to RT. The mixture was filtered and concentrated. The residue was purified by FC (hexane/EtOAc 4:1) to give Int. 2G85 methyl 4-(1-(1-(4-bromo-1-methyl-1H-pyrrolo[2,3-b]pyridin-2-yl)ethyl) piperidin-4-yl)-3-fluoro-5-methylbenzoate (2.0 g).

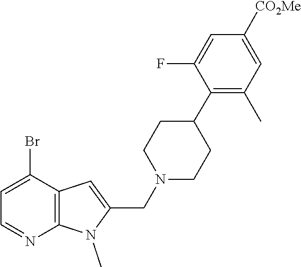 [SMILES: COC(=O)c1cc(C)c(C2CCN(Cc3cc4c(Br)ccnc4n3C)CC2)c(F)c1]

DIPEA (15 mL) was added to a solution of Ints. 1AB8/2G97 (8.0 g/12.5 g) in DCE/DMSO (5:1, 600 mL) at 0° C. and stirred for 16 h at RT. STAB (28 g) was added and stirring continued for 16 h. The OL (water/DCM/MeOH) was dried, filtered, concentrated and purified by FC (hexane/MeOH 4:1 to 2:1) to give Int. 2G115 methyl 4-(1-((4-bromo-1-methyl-1H-pyrrolo[2,3-b]pyridin-2-yl)methyl) piperidin-4-yl)-3-fluoro-5-methylbenzoate (8.0 g).

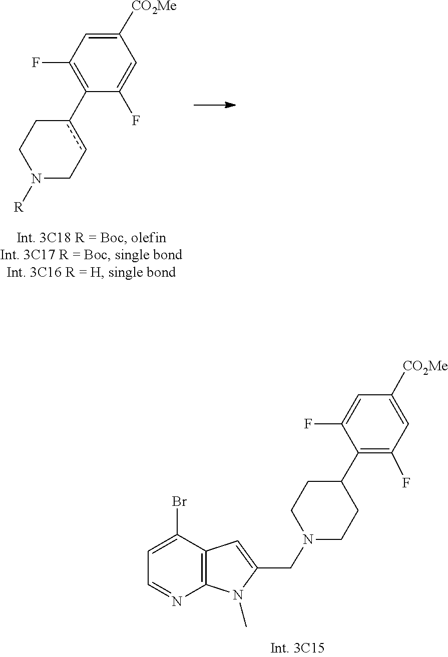

Methyl 4-bromo-3,5-difluorobenzoate (100 g), tert-butyl 4-(4,4,5,5-tetramethyl-1,3,2-dioxaborolan-2-yl)-3,6-dihydropyridine-1(2H)-carboxylate (160 g), PdDPPFCl₂-DCM (16 g), and NaHCO₃ (100 g) were stirred 16 h in dioxane/H₂O (10:3, 1.3 L) at 100° C. under an inert atmosphere. The mixture was filtered. The OL (brine/EtOAc) was dried, concentrated, and purified by FC (PE/EtOAc 1:0 to 9:1) to give Int. 3C₁₈ tert-butyl 4-(2,6-difluoro-4-(methoxycarbonyl)phenyl)-3,6-dihydropyridine-1(2H)-carboxylate (45 g). Int. 3C₁₈ (100 g) and 5% wet Pd/C (20 g) were stirred 16 h in MeOH (0.7 L) under an atmosphere of hydrogen. The mixture was filtered and concentrated to give Int. 3C₁₇ tert-butyl 4-(2,6-difluoro-4-(methoxycarbonyl)phenyl) piperidine-1-carboxylate (90 g). Int. 3C₁₇ (20 g) was stirred 16H in DCM/4M HCl in dioxane (2:1, 600 mL) at 0° C. to RT. The mixture was concentrated and washed with Et₂O to give Int. 3C₁₆ methyl 3,5-difluoro-4-(piperidin-4-yl)benzoate (12 g, HCl salt). Ints. 1AB8/3C₁₆ (3.5 g/3.6 g) were stirred 16 h in DCE/DMSO/DIPEA (7:3:1, 112 mL). STAB (12 g) was added at 0° C. and the mixture was stirred 16 h at 0° C. to RT. The OL (water/DCM) was dried, concentrated, and purified by FC (hexane/EtOAc 9:1) to give Int. 3C₁₅ methyl 4-(1-((4-bromo-1-methyl-1H-pyrrolo[2,3-b]pyridin-2-yl)methyl) piperidin-4-yl)-3,5-difluorobenzoate (4.0 g).

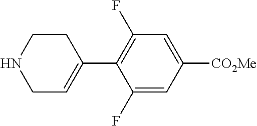 [SMILES: COC(=O)c1cc(F)c(C2=CCNCC2)c(F)c1]

Int. 3C₂₅. methyl 3,5-difluoro-4-(1,2,3,6-tetrahydropyridin-4-yl)benzoate (16 g, HCl salt) was prepared similarly to Int. 3C₁₆ from Int. 3C₁₈ (20 g).

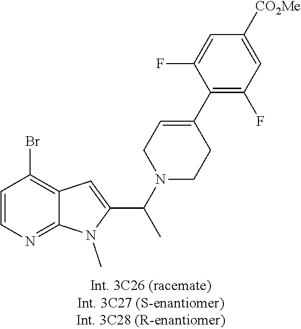 [SMILES: COC(=O)c1cc(F)c(C2=CCN(C(C)c3cc4c(Br)ccnc4n3C)CC2)c(F)c1]

Int. 1AF89 (9.0 g) was stirred 0.5 h in toluene/SOCl₂ (6:1, 174 mL) at 90° C. The residue after concentration, Cs₂CO₃ (43 g), KI (2.7 g), and Int. 3C₂₅ (10 g) were stirred 16 h in ACN (250 mL) at 0° C. to RT under an inert atmosphere. The mixture was concentrated. The OL (water/EtOAc) was dried, concentrated, and purified by FC (PE/EtOAc 4:1) to give Int. 3C₂₆ methyl 4-(1-(1-(4-bromo-1-methyl-1H-pyrrolo[2,3-b]pyridin-2-yl)ethyl)-1,2,3,6-tetrahydropyridin-4-yl)-3,5-difluorobenzoate (7.1 g). Int. 3C₂₆ (0.85 g) was resolved by SFC on a SFC-150-008 instrument fitted with a Chiralpak-AD-H 150×25 mm 5 μm column operated at 30° C. and an eluent of 65% CO₂ and 35% MeOH (80 g/min) and a back pressure of 100 bar to give Int. 3C₂₇ (0.30 g, first peak) and Int. 3C₂₈ (0.30 g, second peak) methyl(S)-4-(1-(1-(4-bromo-1-methyl-1H-pyrrolo[2,3-b]pyridin-2-yl)ethyl)-1,2,3,6-tetrahydropyridin-4-yl)-3,5-difluorobenzoate and methyl (R)-4-(1-(1-(4-bromo-1-methyl-1H-pyrrolo[2,3-b]pyridin-2-yl)ethyl)-1,2,3,6-tetrahydropyridin-4-yl)-3,5-difluorobenzoate. The absolute configurations of these compounds were not determined.

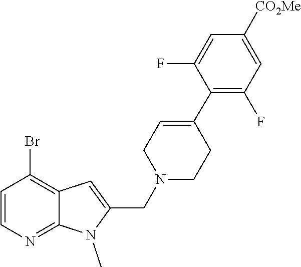 [SMILES: COC(=O)c1cc(F)c(C2=CCN(Cc3cc4c(Br)ccnc4n3C)CC2)c(F)c1]

Ints. 1AB8/3C₂₅ (8.0 g/12.6 g) were stirred 16 h in DCE (80 mL) and DIPEA (35 mL) at 0° C. to RT under an inert atmosphere. STAB (28 g) was added in portions at 0° C. and stirring was continued for 12 h. The OL (water/DCM) was dried, concentrated, and purified by FC (PE/EtOAc 3:2) to give Int. 3C₃₁ methyl 4-(1-((4-bromo-1-methyl-1H-pyrrolo[2,3-b]pyridin-2-yl)methyl)-1,2,3,6-tetrahydropyridin-4-yl)-3,5-difluorobenzoate (7.3 g).

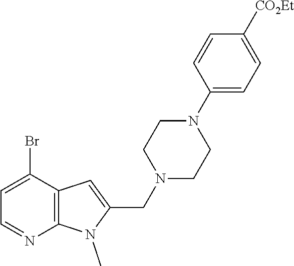

ZnCl₂ (1M in Et₂O, 31 mL) was added to a solution of Int. 1AB8 (2.7 g) and ethyl 4-(piperazin-1-yl)-benzoate (2.4 g) in THF (30 mL) at 0° C. and stirred 16 h at RT. NaCNBH₃ (3.4 g) was added and stirring continued for 16 h. The OL (water/EtOAc) was dried, concentrated, and purified by FC (PE/EtOAc 1:1) to give Int. 3C₅₉ ethyl 4-(4-((4-bromo-1-methyl-1H-pyrrolo[2,3-b]pyridin-2-yl)methyl)-piperazin-1-yl)benzoate (2.2 g).

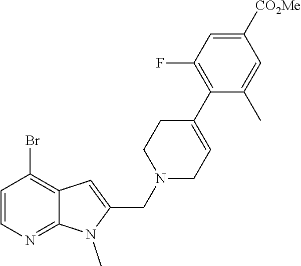 [SMILES: COC(=O)c1cc(C)c(C2=CCN(Cc3cc4c(Br)ccnc4n3C)CC2)c(F)c1]

DIPEA (7.3 mL) was added to a solution of Ints. 1AB8/1AF90 (2.0 g/2.9 g) at 0° C. and stirred in DCE (10 mL) for 16 h at RT under an inert atmosphere. STAB (5.2 g) was added at 0° C. and stirring continued for 2 h. The OL (water/EtOAc) was dried, concentrated, and purified by FC (hexane/EtOAc 3:2) to give Int. 3C₇₃ methyl 4-(1-((4-bromo-1-methyl-1H-pyrrolo[2,3-b]pyridin-2-yl)methyl)-1,2,3,6-tetrahydropyridin-4-yl)-3-fluoro-5-methylbenzoate (1.3 g).

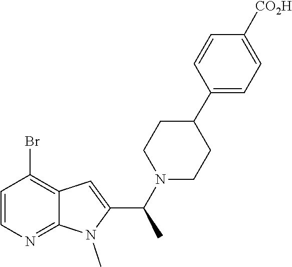 [SMILES: C[C@@H](c1cc2c(Br)ccnc2n1C)N1CCC(c2ccc(C(=O)O)cc2)CC1]

Int. 3E35 (1.7 g) and LiOH—H₂O (1.1 g) were stirred 3 h in THF/MeOH/H₂O (4:2:1, 25 mL) at 0° C. to RT and concentrated. The AL (water/Et₂O) was acidified with KHSO₄ to precipitate Int. 2C3-6 (S)-4-(1-(1-(4-bromo-1-methyl-1H-pyrrolo[2,3-b]pyridin-2-yl)ethyl) piperidin-4-yl)benzoic acid (1.5 g). The absolute configuration was determined for derivatives 1AF76 and 1AF76′.

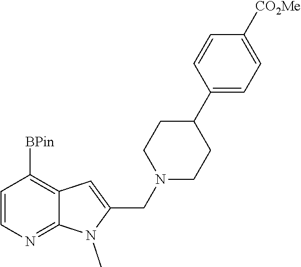

Int. 1AC5 (3.0 g), B₂Pin₂ (1.1 g), KOAc (2.0 g), and PdDPPFCl₂-DCM (50 mg) were degassed in dioxane (30 mL) and stirred ON at 90° C. The filtrate after filtration was concentrated and purified by FC (pentane/EtOAc 1:4) to give Int. 2H14 methyl 4-[1-[1-methyl-4-(4,4,5,5-tetramethyl-1,3,2-dioxaborolan-2-yl) pyrrolo[2,3-b]pyridin-2-yl]methyl]-4-piperidyl]benzoate (1.7 g).

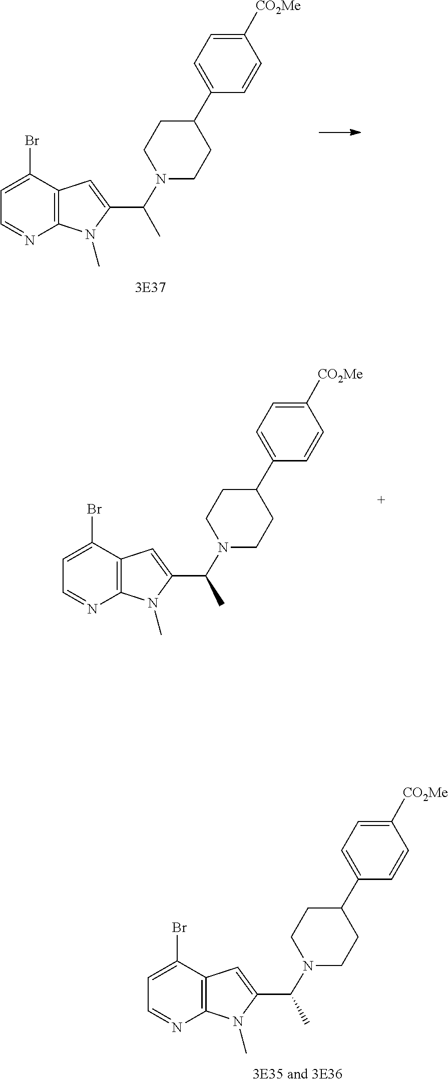 [SMILES: COC(=O)c1ccc(C2CCN(C(C)c3cc4c(Br)ccnc4n3C)CC2)cc1 ; COC(=O)c1ccc(C2CCN([C@@H](C)c3cc4c(Br)ccnc4n3C)CC2)cc1 ; COC(=O)c1ccc(C2CCN([C@H](C)c3cc4c(Br)ccnc4n3C)CC2)cc1]

Int. 1AF21 (1.0 g) was stirred 0.3 h in toluene/SOCl₂ (7.1:1, 22.8 mL) at 85-90° C. The residue after concentration, Cs₂CO₃ (3.8 g), KI (0.33 g), and methyl 4-(piperidin-4-yl)-benzoate (1.3 g, HCl salt) were stirred ON in ACN (25 mL) at 0° C. to RT. The mixture was concentrated to half volume and diluted with water to precipitate Int. 3E37 methyl 4-(1-(1-(4-bromo-1-methyl-1H-pyrrolo[2,3-b]pyridin-2-yl)ethyl)-piperidin-4-yl)benzoate (1.4 g). Int. 3E37 (7.4 g) was resolved by SFC using a Chiralpak IA 250×25 5 μm column operated at 30° C. using an eluent of 60% CO₂ and 40% MeOH at a flow rate of 90 g/min and a back pressure of 100 bar to give Int. 3E35 methyl(S)-4-(1-(1-(4-bromo-1-methyl-1H-pyrrolo[2,3-b]pyridin-2-yl)ethyl) piperidin-4-yl)benzoate (3.0 g, first eluting isomer) and Int. 3E36 methyl (R)-4-(1-(1-(4-bromo-1-methyl-1H-pyrrolo[2,3-b]pyridin-2-yl)ethyl) piperidin-4-yl)benzoate (3.0 g, second eluting isomer). The absolute configuration was determined for derivatives 1AF76 and 1AF76′.

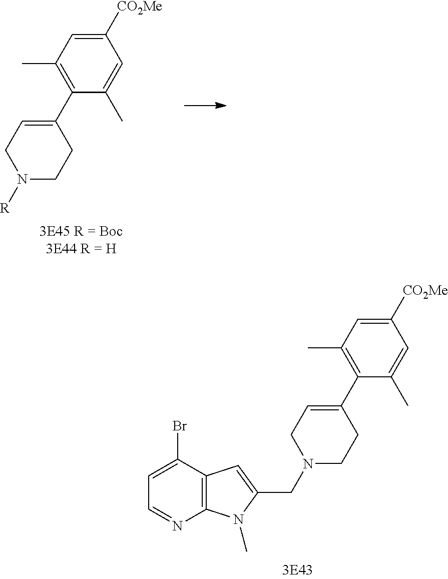

Methyl 4-bromo-3,5-dimethyl-benzoate (0.50 g), tert-butyl 4-(4,4,5,5-tetramethyl-1,3,2-dioxaborolan-2-yl)-3,6-dihydropyridine-1(2H)-carboxylate (0.70 g), Na₂CO₃ (0.26 g), and PdDPPFCl₂-DCM (84 mg) were degassed in dioxane/water (9:1; 8 mL) and stirred ON at 90° C. The OL (water/EtOAc) was concentrated and purified by FC (hexane/EtOAc 7:3) to give Int. 3E45 tert-butyl 4-(4-methoxycarbonyl-2,6-dimethyl-phenyl)-3,6-dihydro-2H-pyridine-1-carboxylate (0.55 g). This material was stirred 3 h in DCM/4M HCl in dioxane (5:4; 9 mL) at 0° C. to RT and concentrated to give Int. 3E44 methyl 3,5-dimethyl-4-(1,2,3,6-tetrahydro-pyridin-4-yl)benzoate (0.44 g, HCl salt). Ints. 1AB8/3E44 (0.23 g/0.30 g) were stirred 4 h in DCE/DIPEA (12.5:1, 5.4 mL). STAB (0.41 g) was added and stirring continued ON. The OL (water/EtOAc) was dried, concentrated, and purified by FC (hexane/EtOAc 7:3) to give Int. 3E43 methyl 4-[1-[(4-bromo-1-methyl-pyrrolo[2,3-b]pyridin-2-yl)methyl]-3,6-dihydro-2H-pyridin-4-yl]-3,5-dimethyl-benzoate (0.33 g).

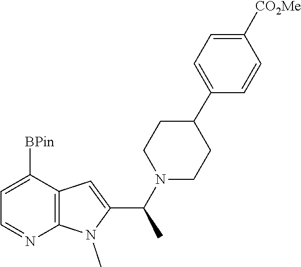

Int. 3E35 (2.0 g), B₂Pin₂ (1.7 g), PdDPPFCl₂-DCM (0.18 g), KOAc (0.9 g) were degassed in dioxane (15 mL) and stirred 5 h at 95-100° C., filtered, concentrated, and triturated in Et₂O to give Int. 3E170 methyl 4-[1-[(1S)-1-[1-methyl-4-(4,4,5,5-tetramethyl-1,3,2-dioxaborolan-2-yl)-pyrrolo[2,3-b]pyridin-2-yl]-ethyl]-4-piperidyl]benzoate (2.3 g).

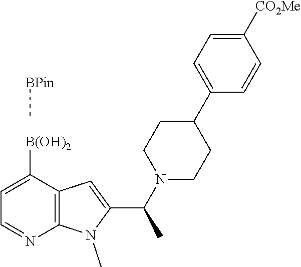

Int. 3E35 (0.50 g), B₂Pin₂ (0.41 g), PdDPPFCl₂-DCM (44 mg), KOAc (0.21 g) were degassed in dioxane (10 mL) and stirred 5 h at 95-100° C., filtered, concentrated, and triturated in Et₂O to give a mixture of Int. 3E170 and 3E177 (S)-(2-(1-(4-(4-(methoxycarbonyl)phenyl) piperidin-1-yl)-ethyl)-1-methyl-1H-pyrrolo[2,3-b]pyridin-4-yl) boronic acid and methyl(S)-4-(1-(1-(1-methyl-4-(4,4,5,5-tetramethyl-1,3,2-dioxaborolan-2-yl)-1H-pyrrolo[2,3-b]pyridin-2-yl)ethyl) piperidin-4-yl)benzoate (0.50 g).

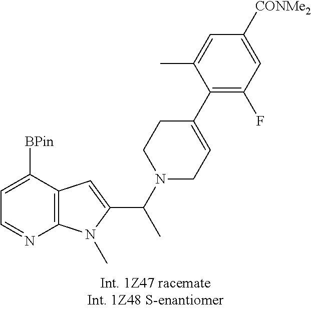

PdDPPFCl₂-DCM (40 mg), Int. 1Z69 (0.24 g), bis (pinacolato)diboron (0.15 g), and KOAc (0.14 g) were stirred ON in dioxane (5 mL) at 100° C. under an inert atmosphere. The mixture was concentrated and purified by FC (THF) to give Int. 1Z47 3-fluoro-N,N,5-trimethyl-4-(1-(1-(1-methyl-4-(4,4,5,5-tetramethyl-1,3,2-dioxaborolan-2-yl)-1H-pyrrolo[2,3-b]pyridin-2-yl)ethyl)-1,2,3,6-tetrahydropyridin-4-yl)benzamide (0.27 g). Int. 1AF84′ (0.20 g), bis (pinacolato)diboron (0.20 g), PdDPPFCl₂-DCM (32 mg), and KOAc (0.12 g) were stirred 6 h in dioxane (10 mL) at 90° C. under an inert atmosphere. The mixture was filtered through celite, concentrated, dissolved in Et₂O, filtered, concentrated, washed with pentane, and dried to give Int. 1Z48 (S)-3-fluoro-5-methyl-N,N-bis(methyl-d3)-4-(1-(1-(1-methyl-4-(4,4,5,5-tetramethyl-1,3,2-dioxaborolan-2-yl)-1H-pyrrolo[2,3-b]pyridin-2-yl)ethyl)-1,2,3,6-tetrahydropyridin-4-yl)benzamide (0.14 g).

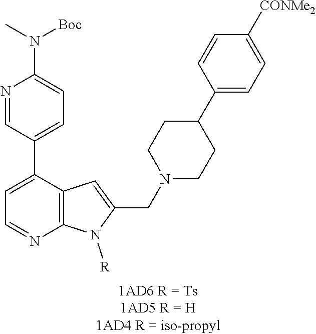

Int. 1AD7 (1.0 g), tert-butyl N-methyl-N-[5-(4,4,5,5-tetramethyl-1,3,2-dioxaborolan-2-y])-2-pyridyl] (0.62 g), PdDPPFCl₂-DCM (0.21 g), and Na₂CO₃ (0.53 g) were degassed in DMF/water (10:1, 11 mL) and stirred at 100° C. ON and filtered. The OL (EtOAc/water) was washed with brine, dried, concentrated, and purified by FC (pentane/EtOAc 1:4) to give Int. 1AD6 tert-butyl N-[5-[2-[4-[4-(dimethylcarbamoyl)-phenyl]-1-piperidyl]-methyl]-1-(p-tolylsulfonyl) pyrrolo[2,3-b]pyridin-4-yl]-2-pyridyl]-N-methyl-carbamate (0.5 g). Int. 1AD6 (1.0 g) was stirred ON in 0.1M TBAF in THE (23 mL) at 70° C. The OL (EtOAc/sat. aq. NaHCO₃) was washed with brine, dried, concentrated, and purified by FC (DCM/MeOH 9:1) to give Int. 1AD5 tert-butyl N-[5-[2-[[4-[4-(dimethyl-carbamoyl)phenyl]-1-piperidyl]methyl]-1H-pyrrolo[2,3-b]pyridin-4-yl]-2-pyridyl]-N-methyl-carbamate (0.4 g). Int. 1AD5 (0.10 g) and NaH (14 mg) were stirred 0.25 h in DMF (5 mL) at 0° C. 2-Bromopropane (0.17 mL) was added and stirring continued ON at 0° C. to RT. The OL (EtOAc/water) was washed with brine, dried, concentrated, and purified by PC (pentane/EtOAc 1:4) to give Int. 1AD4 (60 mg).

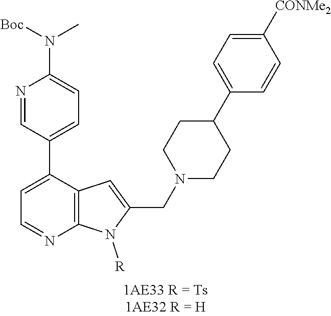

Int. 1AE34 (1.0 g), tert-butyl N-methyl-N-[5-(4,4,5,5-tetramethyl-1,3,2-dioxaborolan-2-yl)-2-pyridyl]-carbamate (0.62 g), PdDPPFCl₂-DCM (0.21 g), and Na₂CO₃ (0.53 g) were degassed in DMF/water (9:1, 20 mL) and stirred ON at 100° C. and filtered. The OL (water/EtOAc) was washed with brine, dried, concentrated, and purified by FC (EtOAc/hexane 4:1) to give Int. 1AE33 tert-butyl N-[5-[2-[4-[4-(dimethylcarbamoyl)-phenyl]-1-piperidyl]-methyl]-1-(p-tolylsulfonyl)-pyrrolo[2,3-b]pyridin-4-yl]-2-pyridyl]-N-methyl-carbamate (0.50 g). Int. 1AE33 (0.45 g) was stirred 3 h in 0.15 M TBAF in THF (16.3 mL) at 70° C. The OL (EtOAc/sat. aq. NaHCO₃.) was dried, concentrated, and purified by PC (DCM/MeOH 9:1) to give Int. 1AE32 tert-butyl N-[5-[2-[[4-[4-(dimethylcarbamoyl)-phenyl]-1-piperidyl]methyl]-1H-pyrrolo[2,3-b]pyridin-4-yl]-2-pyridyl]-N-methyl-carbamate (0.25 g).

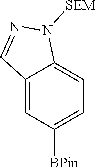

### Int. 1AF16. 5-(4,4,5,5-Tetramethyl-1,3,2-dioxaborolan-2-yl)-1-((2-(trimethylsilyl) ethoxy)methyl)-1H-indazole

SEM-Cl (26.9 mL) was added slowly to a solution of 5-bromo-1H-indazole (20 g) in DCM (0.5 L) and 50% aq. KOH (34.2 g) at 0° C. and stirring continued 5 h at 0° C. to RT. The OL (water/DCM) was dried, concentrated, and purified by FC (pentane/EtOAc 95:5) to give 2-[(5-bromoindazol-1-yl) methoxy]ethyl-trimethyl-silane (17 g). This material, B₂Pin₂ (1.58 g), KOAc (1.52 g), and PdDPPFCl₂-DCM (0.84 g) were degassed in dioxane (300 mL) and stirred ON at 100° C. The mixture was filtered, concentrated, and purified by FC (pentane/EtOAc 95:5) to give Int. 1AF16 (16 g).

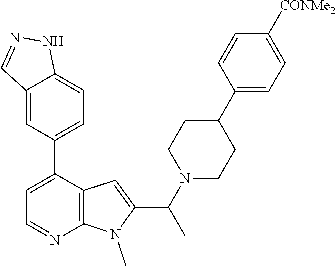

Int. 1AF17 (0.10 g), 1-(tetrahydro-2H-pyran-2-yl)-5-(4,4,5,5-tetramethyl-1,3,2-dioxaborolan-2-yl)-1H-indazole (91 mg), and PdDPPFCl₂-DCM (17 mg) were degassed in DMF/10% aq. Na₂CO₃ (6.7:1, 3.5 mL) and stirred ON at 90° C. The mixture was filtered, concentrated, and purified by FC (hexane/EtOAc 1:4) give Int. 1AF18 4-[1-[1-[4-(6-Amino-3-pyridyl)-1-methyl-pyrrolo[2,3-b]pyridin-2-yl] ethyl]-4-piperidyl]-N,N-dimethyl-benzamide (0.15 g).

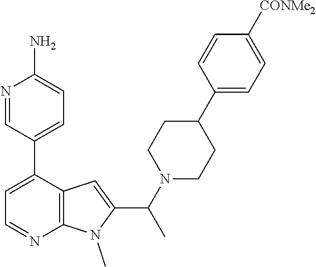

Int. 1AF17 (75 mg), 5-(4,4,5,5-tetramethyl-1,3,2-dioxaborolan-2-yl) pyridin-2-amine (39 mg), Na₂CO₃ (39 mg), and PdCPPFCl₂-DCM (7 mg) were degassed in DMF/water (4:1, 2.5 mL) and stirred ON at 90° C. and filtered. The OL (EtOAc/water) was concentrated and purified by FC (hexane/EtOAc 1:4) to give Int. 1AF19 4-[1-[1-[4-(6-Amino-3-pyridyl)-1-methyl-pyrrolo[2,3-b]pyridin-2-yl]ethyl]-4-piperidyl]-N,N-dimethyl-benzamide (40 mg).

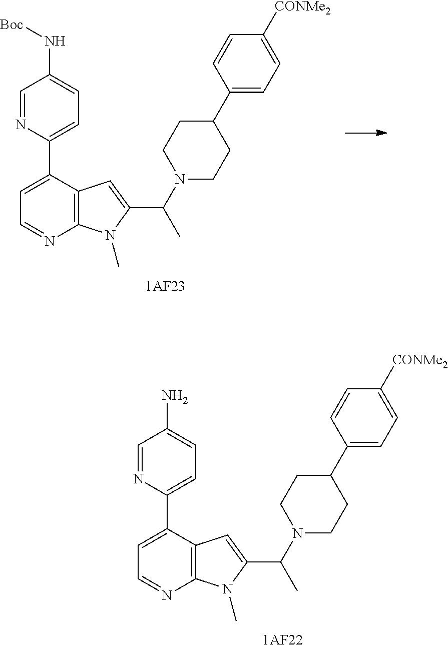

Int. 1AF24 (0.40 g), tert-butyl N-(6-bromo-3-pyridyl)carbamate (0.12 g), PdDPPFCl₂-DCM (38 mg), and K₂CO₃ (0.19 g) were degassed in dioxane/water (4:1, 20 mL) and stirred ON at 100° C. and filtered. The OL (water/EtOAc) was dried, concentrated, and purified by FC (hexane/EtOAc 1:4) to give Int. 1AF23 (0.20 g). Int. 1AF23 (0.50 g) was stirred ON in DCM/TFA (20:1, 21 mL) at 0° C. to RT, concentrated, and HPLC-purified to give Int. 1AF22 4-(1-(1-(4-(5-Aminopyridin-2-yl)-1-methyl-1H-pyrrolo[2,3-b]pyridin-2-yl)ethyl) piperidin-4-yl)-N,N-dimethylbenzamide (80 mg).

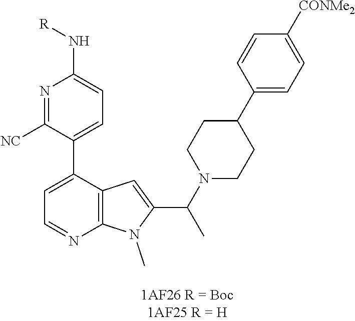

Int. 1AF27 (0.12 g), tert-butyl N-(5-bromo-6-cyano-2-pyridyl)carbamate (69 mg), Na₂CO₃ (39 mg), and PdCPPFCl₂-DCM (7 mg) were degassed in DMF/water (4:1, 2.5 mL) and stirred at 90° C. ON and filtered. The OL (EtOAc/water) was concentrated and purified by FC (hexane/EtOAc 1:4) to give Int. 1AF26 tert-butyl (6-cyano-5-(2-(1-(4-(4-(dimethylcarbamoyl)-phenyl) piperidin-1-yl)ethyl)-1-methyl-1H-pyrrolo[2,3-b]pyridin-4-yl) pyridin-2-yl)carbamate (0.12 g). This material was stirred ON in DCM/TFA (10:1, 11 mL) at 0° C. to RT, concentrated, and HPLC-purified to give Int. 1AF25 4-(1-(1-(4-(6-Amino-2-cyanopyridin-3-yl)-1-methyl-1H-pyrrolo[2,3-b]pyridin-2-yl)ethyl) piperidin-4-yl)-N,N-dimethylbenzamide (23 mg).

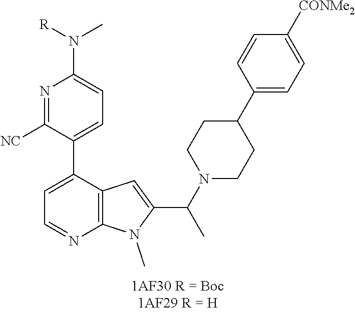

Int. 1AF30 tert-butyl (6-cyano-5-(2-(1-(4-(4-(dimethylcarbamoyl)phenyl) piperidin-1-yl)ethyl)-1-methyl-1H-pyrrolo[2,3-b]pyridin-4-yl) pyridin-2-yl) (methyl)carbamat (0.12 g) was prepared from Int. 1AF27 (0.20 g) and tert-butyl N-(5-bromo-6-cyano-2-pyridyl)-N-methyl-carbamate (0.22 g) similarly to Int. 1AF26. Int. 1AF29 4-(1-(1-(4-(2-Cyano-6-(methylamino)pyridin-3-yl)-1-methyl-1H-pyrrolo[2,3-b]pyridin-2-yl)ethyl) piperidin-4-yl)-N,N-dimethylbenzamide (30 mg) was prepared similarly to Int. 1AF25 from Int. 1AF30 (0.10 g).

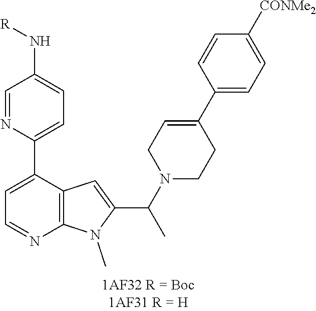

Int. 1AF32 tert-butyl (6-(2-(1-(4-(4-(dimethylcarbamoyl)phenyl)-3,6-dihydropyridin-1(2H)-yl)ethyl)-1-methyl-1H-pyrrolo[2,3-b]pyridin-4-yl) pyridin-3-yl)carbamate (0.28 g) was prepared similarly to Int. 1AF30 from Int. 1AF33 (0.60 g) and tert-butyl N-(6-bromo-3-pyridyl)-carbamate (0.23 g). Int. 1AF31 4-(1-(1-(4-(5-aminopyridin-2-yl)-1-methyl-1H-pyrrolo[2,3-b]pyridin-2-yl)ethyl)-1,2,3,6-tetrahydropyridin-4-yl)-N,N-dimethylbenzamide (60 mg) was prepared similarly to Int. 1AF25 from Int. 1AF32 (0.18 g).

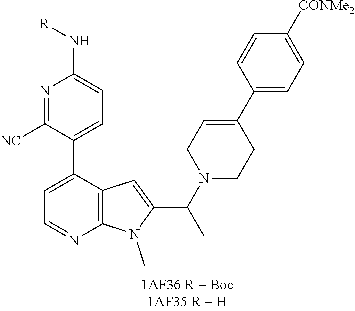

Int. 1AF35. 4-(1-(1-(4-(6-Amino-2-cyanopyridin-3-yl)-1-methyl-1H-pyrrolo[2,3-b]pyridin-2-yl)ethyl)-1,2,3,6-tetrahydropyridin-4-yl)-N,N-dimethylbenzamide (40 mg) was prepared similarly to Int. 1AF25 from Int. 1AF36 (0.12 g). 1AF36 tert-butyl (6-cyano-5-(2-(1-(4-(4-(dimethylcarbamoyl)phenyl)-3,6-dihydropyridin-1(2H)-yl)ethyl)-1-methyl-1H-pyrrolo[2,3-b]pyridin-4-yl) pyridin-2-yl)carbamate (0.14 g) was prepared similarly to Int. 1AF32 from Int. 1AF33 (0.25 g) and tert-butyl N-(5-bromo-6-cyano-2-pyridyl)carbamate (0.13 g).

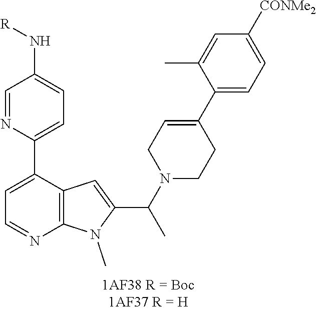

1AF38 tert-butyl N-[6-[2-[1-[4-[4-(dimethylcarbamoyl)-2-methyl-phenyl]-3,6-dihydro-2H-pyridin-1-yl]ethyl]-1-methyl-pyrrolo[2,3-b]pyridin-4-yl]-3-pyridyl]carbamate (50 mg) was prepared similarly to Int. 1AF25 from Int. 1AF39 (0.10 g). Int. 1AF37 4-(1-(1-(4-(5-Aminopyridin-2-yl)-1-methyl-1H-pyrrolo[2,3-b]pyridin-2-yl)ethyl)-1,2,3,6-tetrahydropyridin-4-yl)-N,N,3-trimethylbenzamide (48 mg) was prepared similarly to Int. 1AF25 from Int. 1AF38 (0.15 g).

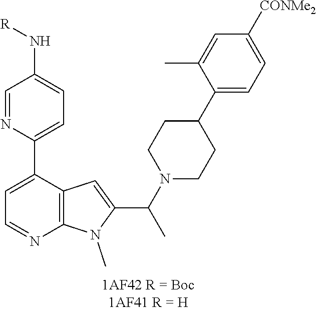

Int. 1AF42 tert-butyl N-[6-[2-[1-[4-[4-(dimethyl-carbamoyl)phenyl]-1-piperidyl]ethyl]-1-methyl-pyrrolo[2,3-b]pyridin-4-yl]-3-pyridyl]carbamate (0.20 g) was prepared similarly to Int. 1AF23 from Int. 1AF43 (0.40 g) and tert-butyl N-(6-bromo-3-pyridyl)carbamate (0.15 g). Int. 1AF41 4-(1-(1-(4-(5-Aminopyridin-2-yl)-1-methyl-1H-pyrrolo[2,3-b]pyridin-2-yl)ethyl) piperidin-4-yl)-N,N,3-trimethylbenzamide (63 mg) was prepared similarly to Int. 1AF25 from Int. 1AF42 (0.20 g).

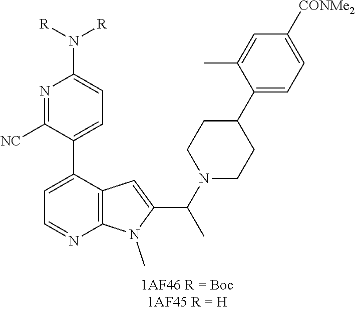

Int. 1AF46 tert-butyl (tert-butoxycarbonyl) (6-cyano-5-(2-(1-(4-(4-(dimethylcarbamoyl)-2-methyl-phenyl)-piperidin-1-yl)ethyl)-1-methyl-1H-pyrrolo[2,3-b]pyridin-4-yl) pyridin-2-yl)carbamate (50 mg) was prepared similarly to Int. 1AF42 from Int. 1AF43 (0.35 g) and di tert-butyl N-(5-bromo-6-cyano-2-pyridyl)carbamate (0.47 g). Int. 1AF45 4-(1-(1-(4-(6-Amino-2-cyanopyridin-3-yl)-1-methyl-1H-pyrrolo[2,3-b]pyridin-2-yl)ethyl) piperidin-4-yl)-N,N,3-trimethylbenzamide (41 mg) was prepared similarly to Int. 1AF25 from Int. 1AF46 (0.14 g).

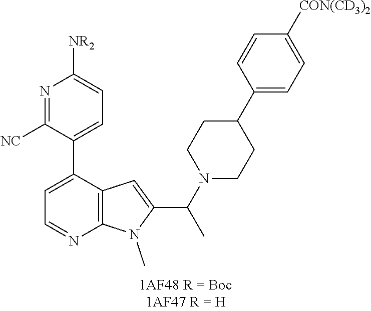

Int. 1AF48 tert-butyl N-[5-[2-[1-[4-[4-[bis(trideuteriomethyl)-carbamoyl]-phenyl]-1-piperidyl]ethyl]-1-methyl-pyrrolo[2,3-b]pyridin-4-yl]-6-cyano-2-pyridyl]-N-tert-butoxycarbonyl-carbamate (0.41 g) was prepared similarly to Int. 1AF26 from Int. 1AF49 (0.35 g) and tert-butyl N-(5-bromo-6-cyano-2-pyridyl)-N-tert-butoxy-carbonyl-carbamate (0.41 g). Int. 1AF47 4-(1-(1-(4-(6-Amino-2-cyanopyridin-3-yl)-1-methyl-1H-pyrrolo[2,3-b]pyridin-2-yl)ethyl) piperidin-4-yl)-N,N-bis(methyl-d₃) benzamide (95 mg) was prepared similarly to Int. 1AF25 from Int. 1AF48 (0.35 g).

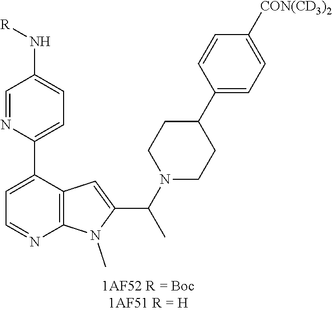

Int. 1AF52 tert-butyl N-[6-[2-[1-[4-[4-[bis(trideuteriomethyl)carbamoyl]phenyl]-1-piperidyl]ethyl]-1-methyl-pyrrolo[2,3-b]pyridin-4-yl]-3-pyridyl]carbamate (0.38 g) was prepared similarly to Int. 1AF42 from Int. 1AF50 (0.35 g) and tert-butyl N-(6-bromo-3-pyridyl)carbamate (0.30 g). Int. 1AF51 4-(1-(1-(4-(5-Aminopyridin-2-yl)-1-methyl-1H-pyrrolo[2,3-b]pyridin-2-yl)ethyl) piperidin-4-yl)-N,N-bis(methyl-d₃) benzamide (92 mg) was prepared similarly to Int. 1AF25 from Int. 1AF52 (0.38 g).

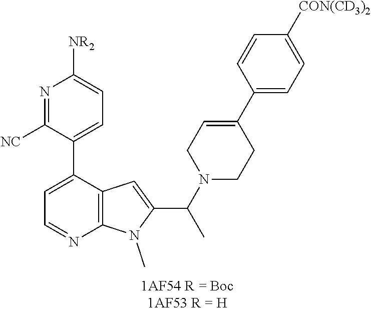

Int. 1AF54 tert-butyl N-[5-[2-[1-[4-[4-[bis(trideuteriomethyl)carbamoyl]phenyl]-3,6-dihydro-2H-pyridin-1-yl]ethyl]-1-methyl-pyrrolo[2,3-b]pyridin-4-yl]-6-cyano-2-pyridyl]-N-tert-butoxy-carbonyl-carbamate (0.45 g) was prepared similarly to from Int. 1AF48 from Int. 1AF55 (0.35 g) and tert-butyl N-(5-bromo-6-cyano-2-pyridyl)-N-tert-butoxycarbonyl-carbamate (0.35 g). Int. 1AF53 4-(1-(1-(4-(6-Amino-2-cyanopyridin-3-yl)-1-methyl-1H-pyrrolo[2,3-b]pyridin-2-yl)ethyl)-1,2,3,6-tetrahydropyridin-4-yl)-N,N-bis(methyl-d₃)-benzamide (60 mg) was prepared similarly to Int. 1AF25 from Int. 1AF54 (0.20 g).

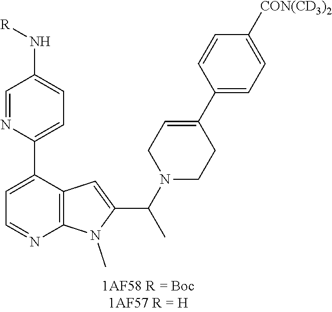

Int. 1AF58 tert-butyl (6-(2-(1-(4-(4-(bis(methyl-d₃)carbamoyl)phenyl)-3,6-dihydropyridin-1(2H)-yl)ethyl)-1-methyl-1H-pyrrolo[2,3-b]pyridin-4-yl) pyridin-3-yl)carbamate (0.20 g) was prepared similarly to like Int. 1AF42 from Int. 1AF55 (0.15 g) and tert-butyl N-(6-bromo-3-pyridyl)carbamate (0.22 g). Int. 1AF57 4-(1-(1-(4-(5-Aminopyridin-2-yl)-1-methyl-1H-pyrrolo[2,3-b]pyridin-2-yl)ethyl)-1,2,3,6-tetrahydropyridin-4-yl)-N,N-bis(methyl-d₃) benzamide (80 mg) was prepared similarly to Int. 1AF25 obtained from Int. 1AF58 (0.15 g).

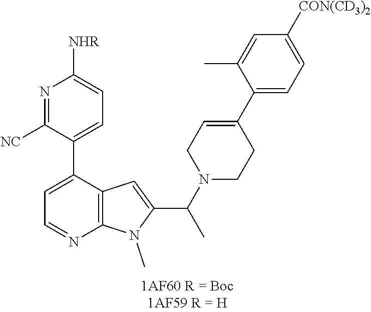

Int. 1AF60 tert-butyl (5-(2-(1-(4-(4-(bis(methyl-d₃)carbamoyl)-2-methyl-phenyl)-3,6-dihydropyridin-1(2H)-yl)ethyl)-1-methyl-1H-pyrrolo[2,3-b]pyridin-4-yl)-6-cyanopyridin-2-yl)-carbamate (0.25 g) was prepared similarly to Int. 1AF32 from Int. 1AF61 (0.40 g) and tert-butyl N-(5-bromo-6-cyano-2-pyridyl)carbamate (0.34 g). Int. 1AF59 4-(1-(1-(4-(6-Amino-2-cyanopyridin-3-yl)-1-methyl-1H-pyrrolo[2,3-b]pyridin-2-yl)ethyl)-1,2,3,6-tetrahydropyridin-4-yl)-3-methyl-N,N-bis(methyl-d₃) benzamide (48 mg) was prepared similarly to Int. 1AF25 from Int. 1AF60 (0.20 g).

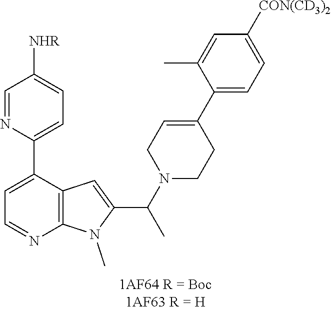

Int. 1AF64 tert-butyl (6-(2-(1-(4-(4-(bis(methyl-d₃)carbamoyl)-2-methylphenyl)-3,6-dihydropyridin-1(2H)-yl)-ethyl)-1-methyl-1H-pyrrolo[2,3-b]pyridin-4-yl) pyridin-3-yl)carbamate (0.22 g) was prepared similarly to Int. 1AF42 from Int. 1AF61 (0.30 g) and tert-butyl N-(6-bromo-3-pyridyl)carbamate (0.16 g). Int. 1AF63 4-(1-(1-(4-(5-Aminopyridin-2-yl)-1-methyl-1H-pyrrolo[2,3-b]pyridin-2-yl)ethyl)-1,2,3,6-tetrahydropyridin-4-yl)-3-methyl-N,N-bis(methyl-d₃) benzamide (38 mg) was prepared similarly to Int. 1AF25 from Int. 1AF64 (0.24 g).

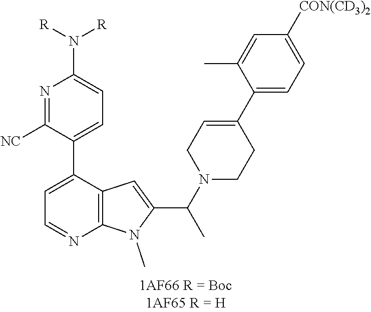

Int. 1AF66 tert-butyl (tert-butoxycarbonyl) (6-cyano-5-(2-(1-(4-(4-(dimethylcarbamoyl)-2-methylphenyl)-3,6-dihydropyridin-1(2H)-yl)ethyl)-1-methyl-1H-pyrrolo[2,3-b]pyridin-4-yl) pyridin-2-yl)carbamate (0.17 g) was prepared similarly to Int. 1AF42 from Int. 1AF39 (0.20 g) and tert-butyl N-(5-bromo-6-cyano-2-pyridyl)-N-tert-butoxycarbonyl-carbamate (0.21 g). Int. 1AF65 4-(1-(1-(4-(6-Amino-2-cyanopyridin-3-yl)-1-methyl-1H-pyrrolo[2,3-b]pyridin-2-yl)ethyl)-1,2,3,6-tetrahydropyridin-4-yl)-N,N,3-trimethylbenzamide (24 mg) was prepared similarly to Int. 1AF25 from Int. 1AF66 (0.17 g).

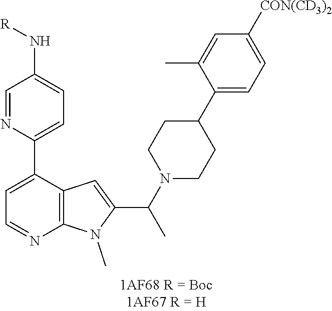

Int. 1AF68 tert-butyl (6-(2-(1-(4-(4-(bis(methyl-d₃)carbamoyl)-2-methylphenyl) piperidin-1-yl)ethyl)-1-methyl-1H-pyrrolo[2,3-b]pyridin-4-yl) pyridin-3-yl)carbamate (0.25 g) was prepared similarly to Int. 1AF42 from Int. 1AF49 (0.30 g) and tert-butyl N-(6-bromo-3-pyridyl)carbamate (0.37 g). Int. 1AF67 4-(1-(1-(4-(5-Aminopyridin-2-yl)-1-methyl-1H-pyrrolo[2,3-b]pyridin-2-yl)ethyl) piperidin-4-yl)-3-methyl-N,N-bis(methyl-ds) benzamide (55 mg) was prepared similarly to Int. 1AF25 from Int. 1AF68 (0.25 g).

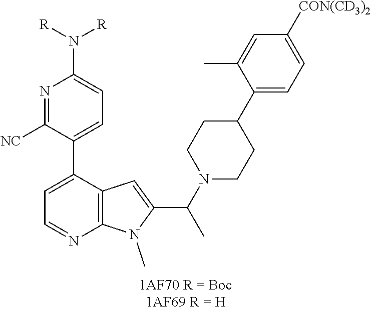

Int. 1AF70 tert-butyl (5-(2-(1-(4-(4-(bis(methyl-d₃)carbamoyl)-2-methylphenyl)-piperidin-1-yl)ethyl)-1-methyl-1H-pyrrolo[2,3-b]pyridin-4-yl)-6-cyanopyridin-2-yl) (tert-butoxy-carbonyl)-carbamate (0.28 g) was prepared similarly to Int. 1AF42 from Int. 1AF71 (0.35 g) and tert-butyl N-(5-bromo-6-cyano-2-pyridyl)-N-tert-butoxycarbonyl-carbamate (0.46 g). Int. 1AF69 4-(1-(1-(4-(6-Amino-2-cyanopyridin-3-yl)-1-methyl-1H-pyrrolo[2,3-b]-pyridin-2-yl)ethyl) piperidin-4-yl)-3-methyl-N,N-bis(methyl-d₃) benzamide (57 mg) was prepared similarly to Int. 1AF25 from Int. 1AF70 (0.23 g).

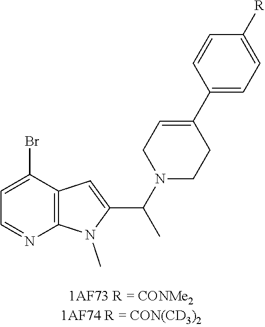

Int. 1AF21 (1.20 g) was stirred 1 h in toluene/SOCl₂ (5.9:1, 23.5 mL) at 0° C. to 110° C. and concentrated. The residue, Cs₂CO₃ (4.60 g), KI (0.39 g), and Int. 1AE7 (1.30 g) were stirred ON in ACN (30 mL) at 0° C. to RT. The OL (water/EtOAc) was washed with brine, dried, concentrated, and purified by FC (hexane/EtOAc 9:1 to 85:15) to give Int. 1AF73 4-[1-[1-(4-bromo-1-methyl-pyrrolo[2,3-b]pyridin-2-yl)ethyl]-3,6-dihydro-2H-pyridin-4-yl]-N,N-dimethyl-benzamide (1.0 g). Int. 1AF74 4-(1-(1-(4-bromo-1-methyl-1H-pyrrolo[2,3-b]pyridin-2-yl)ethyl)-1,2,3,6-tetrahydropyridin-4-yl)-N,N-bis(methyl-ds) benzamide (0.60 g) was prepared similarly from Ints. 1AE7′/1AF21 (0.69 g/0.50 g).

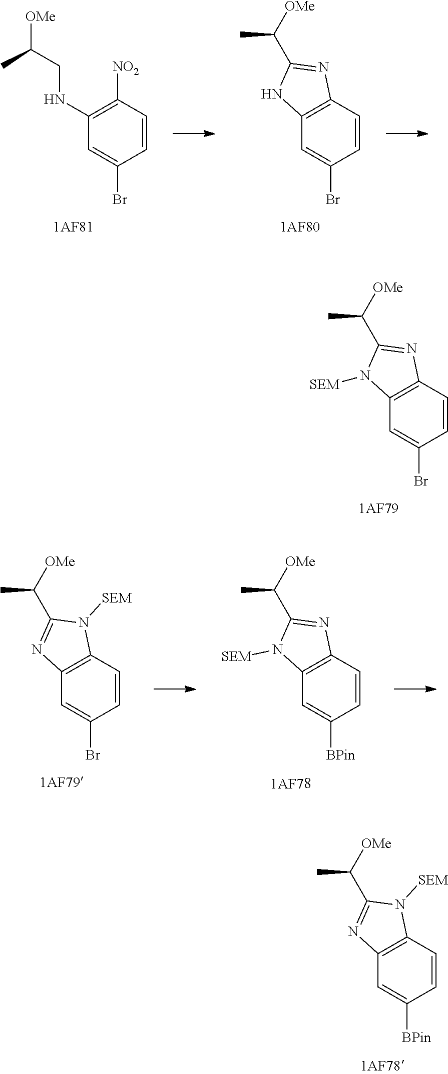

(R)-2-methoxypropanoic acid (2.11 g) was stirred 1 h in pyridine/POCl₃ (17.4:1, 42.6 mL) at 0° C. 4-Bromo-2-nitroaniline (4.0 g) was added and stirring continued ON at 0° C. to RT. The mixture was diluted with aq. HCl to precipitate Int. 1AF81 (R)—N-(4-bromo-2-nitro-phenyl)-2-methoxy-propan-amide (4.0 g). This material and iron powder (2.21 g) were stirred 4 h in AcOH/EtOH (1.8:1, 55 mL) at 65° C. and filtered. The OL (EtOAc/water) was dried, concentrated, and triturated in Et₂O to give Int. 1AF80 5-bromo-2-[(R)-1-methoxyethyl]-1H-benz-imidazole (2.50 g). This material and NaH (0.35 g) were stirred 0.5 h in DMF (5 mL) at 0° C. SEM-Cl (2.0 mL) was added and stirring continued 3 h at 0° C. to RT. The OL (water/EtOAc) was dried, concentrated, and purified by FC (hexane/EtOAc 4:1) to give Ints. 1AF79/1AF79′ (R)-6-bromo-2-(1-methoxy-ethyl)-1-((2-(trimethylsilyl)-ethoxy)methyl)-1H-benzo[d] imidazole and (R)-5-bromo-2-(1-methoxyethyl)-1-((2-(trimethylsilyl) ethoxy)-methyl)-1H-benzo[d]-imidazole (2.0 g). 1.5 g of this mixture, KOAc (1.1 g), 4,4,4′,4′,5,5,5′,5′-octamethyl-2,2′-bi (1,3,2-dioxa-borolane) (2.0 g), and PdDPPFCl₂-DCM (0.32 g) were degassed in dioxane (5 mL) and stirred at 100° C. ON. The OL (water/EtOAc) was dried, concentrated, and purified by FC (hexane/EtOAc 55:45) to give a mixture of Ints. 1AF78 and 1AF78′ (R)-2-(1-methoxyethyl)-6-(4,4,5,5-tetramethyl-1,3,2-dioxaborolan-2-yl)-1-((2-(trimethylsilyl) ethoxy)methyl)-1H-benzo[d]imidazole and (R)-2-(1-methoxyethyl)-5-(4,4,5,5-tetramethyl-1,3,2-dioxaborolan-2-yl)-1-((2-(trimethylsilyl) ethoxy)methyl)-1H-benzo[d]imidazole (1.4 g).

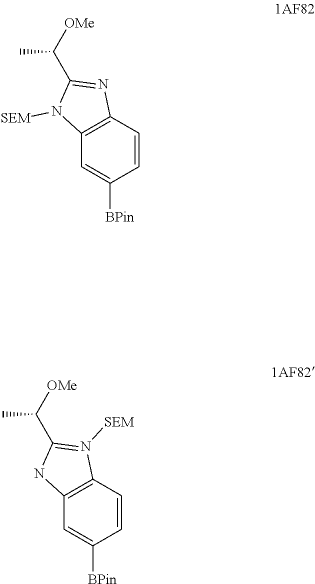

Ints. 1AF82/1AF82′ 2-((S)-1-methoxyethyl)-6-(4,4,5-trimethyl-1,3,2-dioxaborolan-2-yl)-1-((2-(trimethylsilyl) ethoxy)methyl)-1H-benzo[d]imidazole and(S)-2-(1-methoxyethyl)-5-(4,4,5,5-tetramethyl-1,3,2-dioxaborolan-2-yl)-1-((2-(trimethylsilyl) ethoxy)methyl)-1H-benzo[d]imidazole were prepared similarly to Ints. 1AF78/1AF78′ from (R)-2-methoxypropanoic acid.

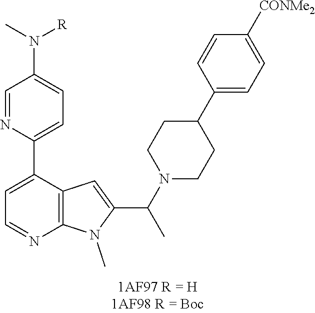

Int. 1AF24 (0.50 g), tert-butyl (6-bromopyridin-3-yl) (methyl)carbamate (0.26 g), PdDPPFCl₂-DCM (56 mg), and K₂CO₃ (0.29 g) were degassed in dioxane/water (4:1, 15 mL) and stirred ON at 100° C. The mixture was filtered. The filtrate was diluted with EtOAc and washed with water. The OL was dried, concentrated, and purified by FC (hexane/EtOAc 1:4) to give Int. 1AF98 tert-butyl N-[6-[2-[1-[4-[4-(dimethylcarbamoyl)phenyl]-1-piperidyl]ethyl]-1-methyl-pyrrolo[2,3-b]pyridin-4-yl]-3-pyridyl]-N-methyl-carbamate (0.16 g). Int. 1AF98 (0.15 g) was stirred ON in DCM/TFA (8:1, 9 mL) at 0° C. to RT. The mixture was concentrated and triturated with pentane to give a solid. This material was HPLC-purified to give Int. 1AF97 N,N-dimethyl-4-(1-(1-(1-methyl-4-(5-(methylamino)pyridin-2-yl)-1H-pyrrolo[2,3-b]pyridin-2-yl)ethyl) piperidin-4-yl)benzamide (22 mg).

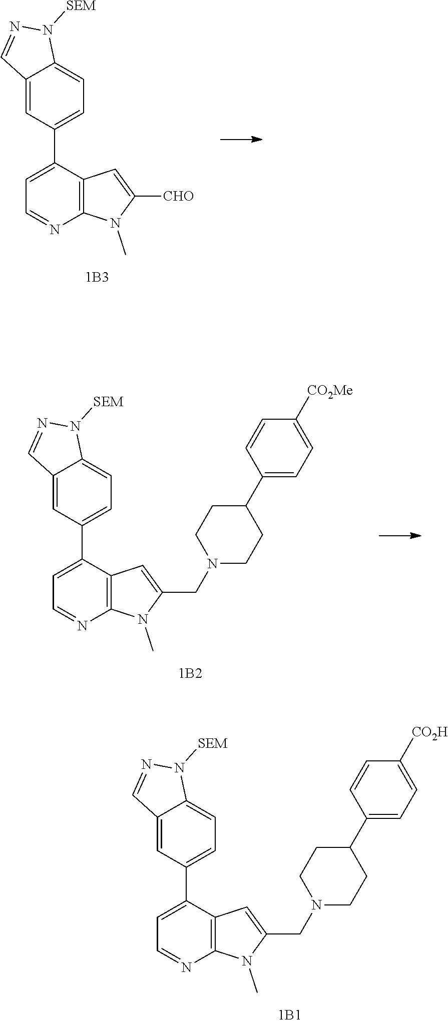 [SMILES: Cn1c(C=O)cc2c(-c3ccc4c(cnn4COCC[Si](C)(C)C)c3)ccnc21 ; COC(=O)c1ccc(C2CCN(Cc3cc4c(-c5ccc6c(cnn6COCC[Si](C)(C)C)c5)ccnc4n3C)CC2)cc1 ; Cn1c(CN2CCC(c3ccc(C(=O)O)cc3)CC2)cc2c(-c3ccc4c(cnn4COCC[Si](C)(C)C)c3)ccnc21]

PdDPPFCl₂-DCM (0.34 g), Int. 1AB8 (2.0 g), trimethyl-[2-[[5-(4,4,5,5-tetramethyl-1,3,2-dioxaborolan-2-yl)-indazol-1-yl] methoxy]ethyl] silane (4.7 g), and NaHCO₃ (2.2 g) were degassed in dioxane/water (5:1, 24 mL) and stirred ON at 110° C. The OL (water/EtOAc) was washed with brine, dried, concentrated, and purified by FC (hexane/EtOAc 1:1) to give Int. 1B3 1-methyl-4-(1-((2-(trimethylsilyl) ethoxy)-methyl)-1H-indazol-5-yl)-1H-pyrrolo[2,3-b]pyridine-2-carbaldehyde (2.2 g). N,N-Dimethyl-4-(4-piperidinyl)-benzamide (0.58 g, HCl salt), Int. 1B3 (0.62 g), and 4 Å MS were stirred ON in DCM/AcOH (5:1, 6 mL). STAB (0.65 g) was added and stirring continued 2 h. The mixture was filtered, concentrated, and purified by FC (heptane to EtOAc) to give Int. 1B2 methyl 4-(1-((1-methyl-4-(1-((2-(tri-methylsilyl) ethoxy)methyl)-1H-indazol-5-yl)-1H-pyrrolo[2,3-b]pyridin-2-yl)methyl)-piperidin-4-yl)-benzoate (0.31 g). Int. 1B2 (88 mg) and LiOH (10 mg) were stirred ON in MeOH/water (2:1, 1 mL) at 50° C. The mixture was partially concentrated. The OL (aq. citric acid/EtOAc) was washed with brine, dried, and concentrated to give Int. 1B1 4-(1-((1-Methyl-4-(1-((2-(trimethylsilyl) ethoxy)methyl)-1H-indazol-5-yl)-1H-pyrrolo[2,3-b]pyridin-2-yl)methyl) piperidin-4-yl)benzoic acid (83 mg).

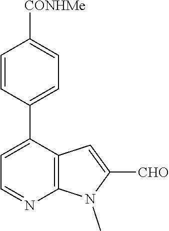

Int. 1AB8 (0.15 g), (4-(methylcarbamoyl)phenyl) boronic acid (0.17 g), PdDPPFCl₂-DCM (51 mg), and NaHCO₃ (0.16 g) were degassed in dioxane/water (2.4:1, 4.4 mL) and stirred 0.5 h at 100° C. The OL (water/EtOAc) was washed with brine, dried, concentrated, and purified by FC (DCM/MeOH 1:0 to 95:5) to give Int. 1J2 4-(2-formyl-1-methyl-1H-pyrrolo[2,3-b]pyridin-4-yl)-N-methylbenzamide (64 mg).

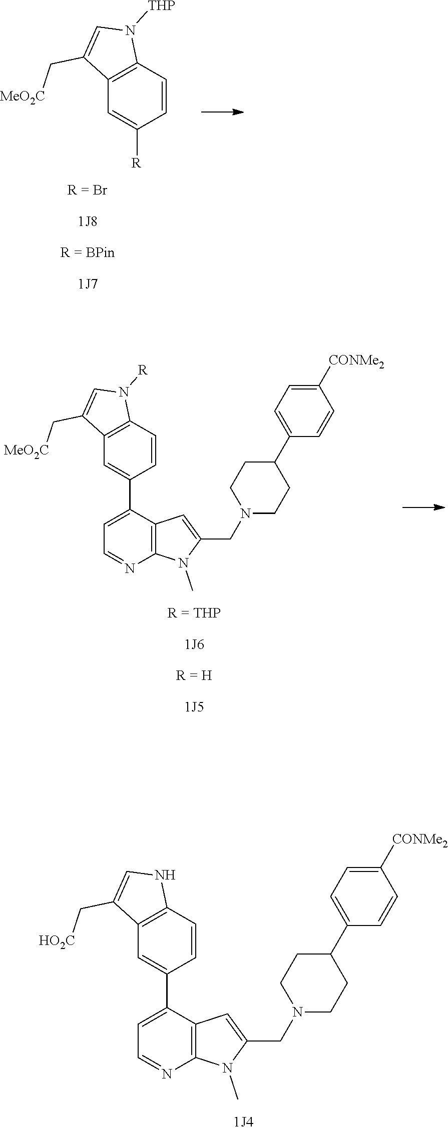

Methyl 2-(5-bromo-1H-indol-3-yl) acetate (30 g), 3,4-dihydro-2H-pyran (14.1 g), and TsOH-H₂O (4.26 g) were stirred in DCM (0.3 L) ON. The OL (DCM/water) was dried, concentrated, and purified by FC (pentane/EtOAc 1:0 to 85:15) to give Int. 1J8 methyl 2-(5-bromo-1-tetrahydropyran-2-yl-indol-3-yl) acetate (18 g). 5.0 g of this material, B₂Pin₂ (4.3 g), KOAc (2.8 g), and PdDPPFCl₂-DCM (0.52 g) were degassed in dioxane (50 mL) and stirred ON at 110° C. The mixture was filtered, concentrated, and purified by FC (pentane/EtOAc 1:0 to 7:3) to give Int. 1J7 methyl 2-(1-(tetrahydro-2H-pyran-2-yl)-5-(4,4,5,5-tetramethyl-1,3,2-dioxaborolan-2-yl)-1H-indol-3-yl) acetate (4.8 g). Ints. 1J7/1AB1 (4.5 g/3.0 g), NaHCO₃ (1.66 g), and PdDPPFCl₂-DCM (0.24 g) were degassed in dioxane/water (6.7:1, 23 mL) and stirred ON at 100° C. The mixture was filtered, concentrated, and purified by FC (DCM/MeOH 97:3 to 95:5) to give Int. 1J6 methyl 2-(5-(2-((4-(4-(dimethyl-carbamoyl)phenyl)-piperidin-1-yl)methyl)-1-methyl-1H-pyrrolo[2,3-b]pyridin-4-yl)-1-(tetrahydro-2H-pyran-2-yl)-1H-indol-3-yl)acetate (3.0 g). 15 mg of this material was stirred 3 h in DCM/TFA (1:1, 2 mL), concentrated, and HPLC-purified to give Int. 1J5 methyl 2-(5-(2-((4-(4-(dimethylcarbamoyl)-phenyl) piperidin-1-yl)methyl)-1-methyl-1H-pyrrolo[2,3-b]pyridin-4-yl)-1H-indol-3-yl)acetate (5 mg). This material was stirred 1 h in MeOH/1M aq. LiOH (1:1, 2 mL), neutralized, and HPLC-purified to give Int. 1J4 2-(5-(2-((4-(4-(dimethylcarbamoyl)phenyl) piperidin-1-yl)methyl)-1-methyl-1H-pyrrolo[2,3-b]pyridin-4-yl)-1H-indol-3-yl) acetic acid (3.9 mg).

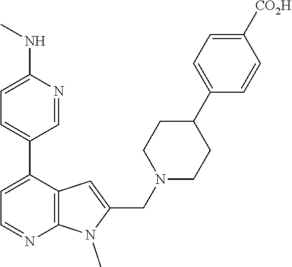 [SMILES: CNc1ccc(-c2ccnc3c2cc(CN2CCC(c4ccc(C(=O)O)cc4)CC2)n3C)cn1]

Int. 1AC4 (0.30 g, HCl salt), N-methyl-5-(4,4,5,5-tetramethyl-1,3,2-dioxaborolan-2-yl) pyridin-2-amine (0.17 g), PdDPPFCl₂-DCM (53 mg), K₂CO₃ (0.47 g) were degassed in DMF/water (8:1, 4.2 mL) and stirred at 100° C. ON. The reaction was repeated at the same scale. The combined residues after concentration of the filtrates were purified by HPLC to give Int. 1L1 4-[1-[1-Methyl-4-[6-(methylamino)-3-pyridyl] pyrrolo[2,3-b]pyridin-2-yl]methyl]-4-piperidyl]benzoic acid (0.29 g).

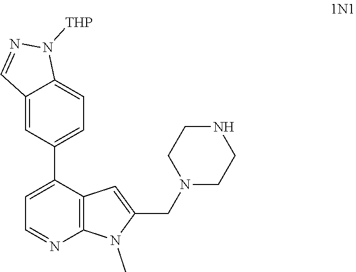

Int. 1N2 (0.15 g), K₂CO₃ (34 mg), Int. 1AF16 (0.3 g), and PdDPPFCl₂-DCM (7 mg) were degassed in DMF/water (10:1, 2.2 mL) and stirred 4 h at 90° C. and filtered. The OL (water/EtOAc) was concentrated, and purified by FC (heptane/EtOAc 7:3) to give 9H-fluoren-9-ylmethyl 4-[1-methyl-4-(1-tetrahydropyran-2-ylindazol-5-yl) pyrrolo[2,3-b]pyridin-2-yl]methyl]-piperazine-1-carboxylate (0.18 g). This material was combined with another batch prepared on 8 g scale and stirred 2 h in DMF/piperidine (1.3:1, 40 mL) at 0° C. to RT, concentrated, and purified by FC (DCM/MeOH 9:1) to give Int. 1N1 5-(1-Methyl-2-(piperazin-1-ylmethyl)-1H-pyrrolo[2,3-b]pyridin-4-yl)-1-(tetrahydro-2H-pyran-2-yl)-1H-indazole (2.5 g).

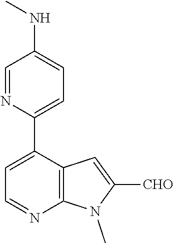 [SMILES: CNc1ccc(-c2ccnc3c2cc(C=O)n3C)nc1]

4,4,5,5-Tetramethyl-2-(4,4,5,5-tetramethyl-1,3,2-dioxaborolan-2-yl)-1,3,2-dioxaborolane (0.27 g), Int. 1AB8 (0.1 g), KOAc (82 mg), and PdDPPFCl₂-DCM (15 mg) were degassed in dioxane (2.5 mL) and stirred at 100° C. ON. The mixture was filtered and HPLC-purified to give a mixture of 1-methyl-4-(4,4,5,5-tetramethyl-1,3,2-dioxaborolan-2-yl) pyrrolo[2,3-b]pyridine-2-carbaldehyde and (2-formyl-1-methyl-pyrrolo[2,3-b]pyridin-4-yl) boronic acid (43 mg). 0.35 g of this mixture, 6-bromo-N-methylpyridin-3-amine (0.39 g), PdDPPFCl₂-DCM (0.14 g), and K₂CO₃ (0.71 g) were degassed in DMF/water (5:1, 0.7 mL) and stirred 1 h at 100° C. The OL (EtOAc/water) was washed with brine, dried, concentrated, and purified by FC (heptane/EtOAc 1:0 to 7:3) to give Int. 1N6/1O4 1-methyl-4-[5-(methylamino)-2-pyridyl] pyrrolo[2,3-b]pyridine-2-carbaldehyde (0.21 g).

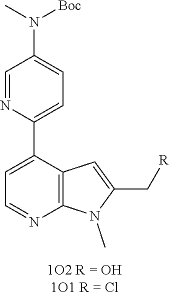

Int. 1O3 (0.38 g), tert-butyl (6-bromopyridin-3-yl) (methyl)carbamate (0.35 g), PdDPPFCl₂-DCM (0.10 g), and K₂CO₃ (0.50 g) were degassed in dioxane/water (5:1, 4.8 mL) and stirred at 110° C. ON. The OL (water/EtOAc) was washed with brine, concentrated, purified by FC (EtOAc) to give Int. 102 tert-butyl N-[6-[2-(hydroxymethyl)-1-methyl-pyrrolo[2,3-b]-pyridin-4-yl]-3-pyridyl]-N-methyl-carbamate (0.15 g). Int. 1O2 (90 mg) was stirred 2 h in DCM/Et₃N (80:1, 8 mL) and MsCl (28 uL) at 0° C. to RT. The OL (water/EtOAc) was washed with sat. aq. NaHCO₃, dried, and concentrated to give Int. 101 tert-butyl (6-(2-(chloromethyl)-1-methyl-1H-pyrrolo[2,3-b]pyridin-4-yl) pyridin-3-yl) (methyl)-carbamate (90 mg).

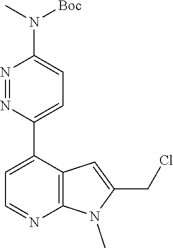 [SMILES: CN(C(=O)OC(C)(C)C)c1ccc(-c2ccnc3c2cc(CCl)n3C)nn1]

Int. 1O₃ (0.54 g), PdDPPFCl₂-DCM (0.14 g), and tert-butyl N-(6-bromopyridazin-3-yl)-N-methyl-carbamate (0.5 g) were degassed in dioxane/water (4.3:1, 8 mL) and stirred at 110° C. ON. The mixture was filtered, concentrated, and purified by FC (hexane/EtOAc 3:7) to give tert-butyl N-[6-[2-(hydroxymethyl)-1-methyl-pyrrolo[2,3-b]pyridin-4-yl]pyridazin-3-yl]-N-methyl-carbamate (0.5 g). 0.2 g of this material was stirred in DCM/Et₃N (11.4:1, 5.5 mL) and MsCl (0.13 mL) at 0° C. to RT and concentrated to give Int. 1P1 tert-butyl N-[6-[2-(chloromethyl)-1-methyl-pyrrolo[2,3-b]pyridin-4-yl] pyridazin-3-yl]-N-methyl-carbamate (0.18 g).

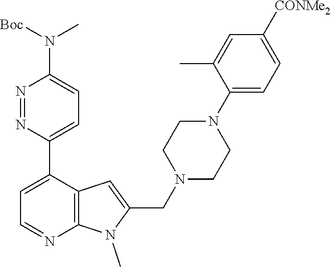

Ints. 1S7/1P1 (0.26 g/0.20 g, TFA salt) and KI (9 mg) were stirred ON in DMF/DIPEA (6.9:1, 2.3 mL) at 0° C. to RT. The OL (EtOAc/water) was dried, concentrated, and purified by FC (DCM/MeOH 9:1) to give Int. 1P2 tert-butyl (6-(2-((4-(4-(dimethylcarbamoyl)-2-methylphenyl) piperazin-1-yl)methyl)-1-methyl-1H-pyrrolo[2,3-b]pyridin-4-yl)pyridazin-3-yl) (methyl)carbamate (0.2 g)

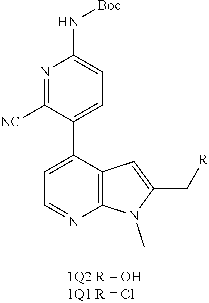

Int. 1O₃ (0.69 g), tert-butyl (5-bromo-6-cyanopyridin-2-yl)carbamate (0.50 g), PdDPPFCl₂-DCM (0.14 g), and K₂CO₃ (0.69 g) were degassed in dioxane/water (5:1, 12 mL) and stirred ON at 110° C. The OL (EtOAc/water) was washed with brine, dried, concentrated, and purified by FC (EtOAc) to give Int. 1Q2 tert-butyl N-[6-cyano-5-[2-(hydroxymethyl)-1-methyl-pyrrolo[2,3-b]pyridin-4-yl]-2-pyridyl]carbamate (0.40 g). Int. 1Q2 (0.10 g) was stirred 2 h in DCM/Et₃N (53:1, 8.2 mL) and MsCl (0.03 mL). The OL (EtOAc/water) was washed with sat. aq. NaHCO₃ and water, dried, and concentrated to give Int. 1Q1 tert-butyl N-[5-[2-(chloromethyl)-1-methyl-pyrrolo[2,3-b]pyridin-4-yl]-6-cyano-2-pyridyl]carbamate (0.10 g).

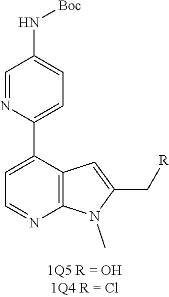

Int. 1O₃ (0.40 g), tert-butyl N-(6-bromo-3-pyridyl)carbamate (0.80 g), PdDPPFCl₂-DCM (0.16 g), and K₂CO₃ (0.8 g) were degassed in dioxane/water (5:1, 6 mL) and stirred ON at 110° C. The OL (EtOAc/water) was washed with brine, dried, concentrated, and purified by FC (EtOAc) to give Int. 1Q5 tert-butyl N-[6-[2-(hydroxymethyl)-1-methyl-pyrrolo[2,3-b]pyridin-4-yl]-3-pyridyl]-carbamate (0.35 g). Int. 1Q5 (0.10 g) was stirred 2 h in DCM/Et₃N (50:1, 5.1 mL) and MsCl (34 uL). The OL (EtOAc/water) was washed with sat. aq. NaHCO₃, dried, and concentrated to give Int. 1Q4 tert-butyl N-[6-[2-(chloromethyl)-1-methyl-pyrrolo[2,3-b]pyridin-4-yl]-3-pyridyl]carbamate (0.10 g).

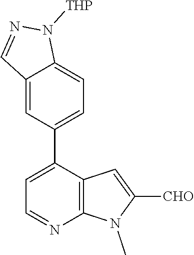

PdDPPFCl₂-DCM (1.37 g), 4-bromo-1-methyl-1H-pyrrolo[2,3-b]pyridine-2-carbaldehyde (8 g), and 1-(tetra-hydro-2H-pyran-2-yl)-5-(4,4,5,5-tetramethyl-1,3,2-dioxaborolan-2-yl)-1H-indazole (13.2 g) were stirred ON in 2M aq. NaHCO₃/dioxane/water (2.3:5:3:1, 130 mL) at 110° C. ON, filtered, and diluted with EtOAc. The OL was dried, concentrated, and purified by FC (hexane/EtOAc 1:3) to give Int. 1R7 1-methyl-4-(1-(tetrahydro-2H-pyran-2-yl)-1H-indazol-5-yl)-1H-pyrrolo[2,3-b]pyridine-2-carbaldehyde (3.8).

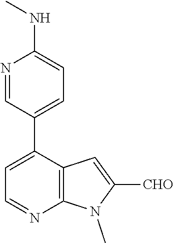 [SMILES: CNc1ccc(-c2ccnc3c2cc(C=O)n3C)cn1]

Int. 1AB8 (0.40 g), NaHCO₃ (0.28 g), N-methyl-5-(4,4,5,5-tetramethyl-1,3,2-dioxaborolan-2-yl) pyridin-2-amine (0.47 g), and PdDPPFCl₂-DCM (0.14 g) were degassed in dioxane/water (9:1, 10 mL) and stirred 2 h at 110° C. under MW conditions, concentrated, and purified by FC (hexane/EtOAc 1:1) to give Int. 1S1 1-methyl-4-[6-(methylamino)-3-pyridyl] pyrrolo[2,3-b]pyridine-2-carbaldehyde (0.32 g).

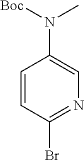 [SMILES: CN(C(=O)OC(C)(C)C)c1ccc(Br)nc1]

tert-Butyl N-(6-bromo-3-pyridyl)carbamate (5.0 g), Cs₂CO₃ (18.0 g) were stirred 2 h in DMF/CH₃I (6.2:1, 17.3 mL) at 0° C. to 50° C. The OL (EtOAc/water) was washed with water, brine, dried, concentrated, and purified by FC (pentane/EtOAc 95:5) to give Int. 1Y⁴ tert-butyl (6-bromopyridin-3-yl) (methyl)carbamate (4.8 g).

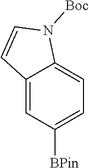

tert-Butyl 5-bromo-1H-indole-1-carboxylate (7.0 g), B₂Pin₂ (7.2 g), PdDPPFCl₂-DCM (0.97 g), and KOAc (7.0 g) were degassed in dioxane/water (11.7:1, 76 mL) and stirred at 100° C. ON. The mixture was filtered, concentrated, and purified by FC (hexane/EtOAc 1:0 to 95:5) to give Int. 175 tert-butyl 5-(4,4,5,5-tetramethyl-1,3,2-dioxaborolan-2-yl)-1H-indole-1-carboxylate (3.7 g).

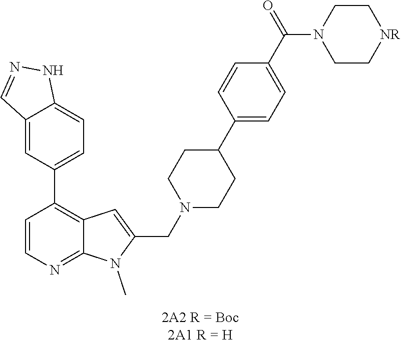

Int. 3A2 (0.20 g, HCl salt), tert-butyl piperazine-1-carboxylate (64 mg), and HATU (0.16 g) were stirred 2 h in DMF/DIPEA (5.7:1, 4 mL). The OL (EtOAc/water) was washed with brine, dried, concentrated, and HPLC-purified to give Int. 2A2 tert-butyl 4-[4-[1-[[1-methyl-4-(1-tetrahydro-pyran-2-ylindazol-5-yl) pyrrolo[2,3-b]-pyridin-2-yl]methyl]-4-piperidyl]benzoyl]-piperazine-1-carboxylate (0.21 g). This material was stirred 1 h in DCM/TFA (2:1, 6 mL). The mixture purified by SCX to give Int. 2A1 (4-(1-((4-(1H-indazol-5-yl)-1-methyl-1H-pyrrolo[2,3-b]pyridin-2-yl)methyl) piperidin-4-yl)phenyl) (piperazin-1-yl) methanone (0.15 g).

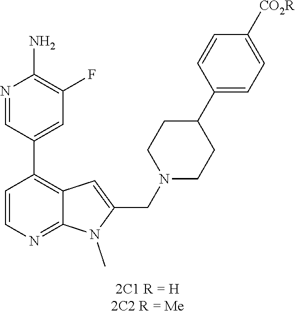

Int. 1AC5 (0.20 g), 3-fluoro-5-(4,4,5,5-tetramethyl-1,3,2-dioxaborolan-2-yl) pyridin-2-amine (0.34 g), PdDPPFCl₂-DCM (55 mg), and NaHCO₃ (0.19 g) were deassed in dioxane/water (10:1, 2.7 mL) and stirred ON at 90° C. The residue after filtration and concentration was purified by FC (heptane/EtOAc/MeOH 1:0:0 to 0:95:5) to give Int. 2C2 methyl 4-[1-[4-(6-amino-5-fluoro-3-pyridyl)-1-methyl-pyrrolo[2,3-b]pyridin-2-yl]methyl]-4-piperidyl]-benzoate (0.19 g). This material was stirred 1 h in THF/MeOH/2M aq. NaOH (4:2:1, 7 mL). 2M aq. NaOH (1 mL) was added and stirring continued ON and concentrated. The AL (EtOAc/water) was acidified with aq. HCl to precipitate Int. 2C1-4-(1-((4-(6-amino-5-fluoropyridin-3-yl)-1-methyl-1H-pyrrolo[2,3-b]pyridin-2-yl)methyl)-piperidin-4-yl)benzoic acid (0.15 g) after partial concentration.

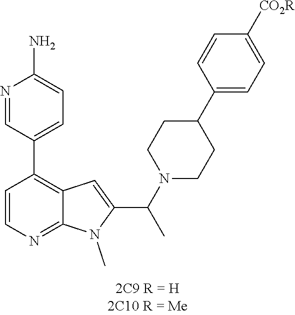

Int. 3E37 (0.15 g), 5-(4,4,5,5-tetramethyl-1,3,2-dioxaborolan-2-yl) pyridin-2-amine (0.11 g), PdDPPFCl₂-DCM (27 mg), and Na₂CO₃ (0.14 g) were degassed in dioxane/water (7:1, 3.6 mL and stirred overnight at 90° C. The OL (EtOAc/brine) was dried, concentrated, and purified by FC (heptane to EtOAc to EtOAc: MeOH (3:2)) to give Int. 2C10 methyl 4-[1-[1-[4-(6-amino-3-pyridyl)-1-methyl-pyrrolo[2,3-b]pyridin-2-yl]ethyl]-4-piperidyl]benzoate (153 mg). This material was stirred ON in THF/MeOH/2M aq. NaOH (1.8:0.9:1, 3.7 mL), neutralized, and HPLC-purified to give Int. 2C9 4-(1-(1-(4-(6-aminopyridin-3-yl)-1-methyl-1H-pyrrolo[2,3-b]pyridin-2-yl)ethyl) piperidin-4-yl)benzoic acid (95 mg).

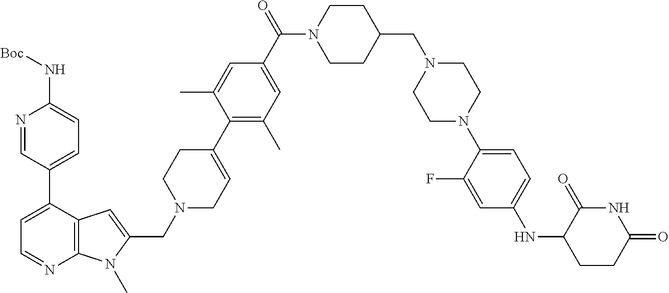 [SMILES: Cc1cc(C(=O)N2CCC(CN3CCN(c4ccc(NC5CCC(=O)NC5=O)cc4F)CC3)CC2)cc(C)c1C1=CCN(Cc2cc3c(-c4ccc(NC(=O)OC(C)(C)C)nc4)ccnc3n2C)CC1]

Ints. 3E41/2C15 (20/16 mg) and HATU (16 mg) were stirred 2 h in DCM/DIPEA/DMF (6:1:1.2, 0.41 mL). The OL (sat. aq. NaHCO₃/DCM) was concentrated and purified by FC (DCM/MeOH 1:0 to 96:4) to give Int. 2C18 tert-butyl (5-(2-((4-(4-(4-((4-(4-((2,6-dioxopiperidin-3-yl) amino)-2-fluorophenyl) piperazin-1-yl)methyl) piperidine-1-carbonyl)-2,6-dimethylphenyl)-3,6-dihydropyridin-1(2H)-yl)methyl)-1-methyl-1H-pyrrolo[2,3-b]pyridin-4-yl) pyridin-2-yl)carbamate (22 mg).

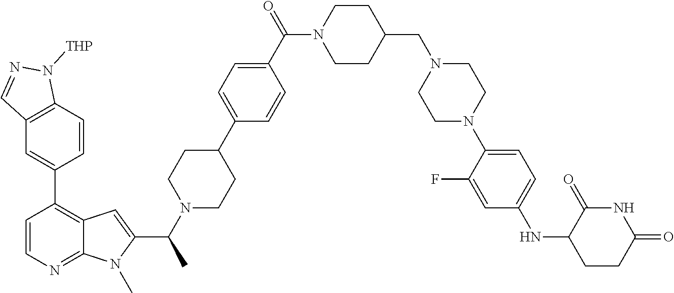

Int. 2C19 3-((3-fluoro-4-(4-((1-(4-(1-((1S)-1-(1-methyl-4-(1-(tetrahydro-2H-pyran-2-yl)-1H-indazol-5-yl)-1H-pyrrolo[2,3-b]pyridin-2-yl)ethyl) piperidin-4-yl)benzoyl) piperidin-4-yl)methyl) piperazin-1-yl)phenyl) amino)-piperidine-2,6-dione (0.19 g) prepared similarly to Int. 2C18 from Ints. 2C54/2C15 (0.12 g/0.12 g, TFA salt).

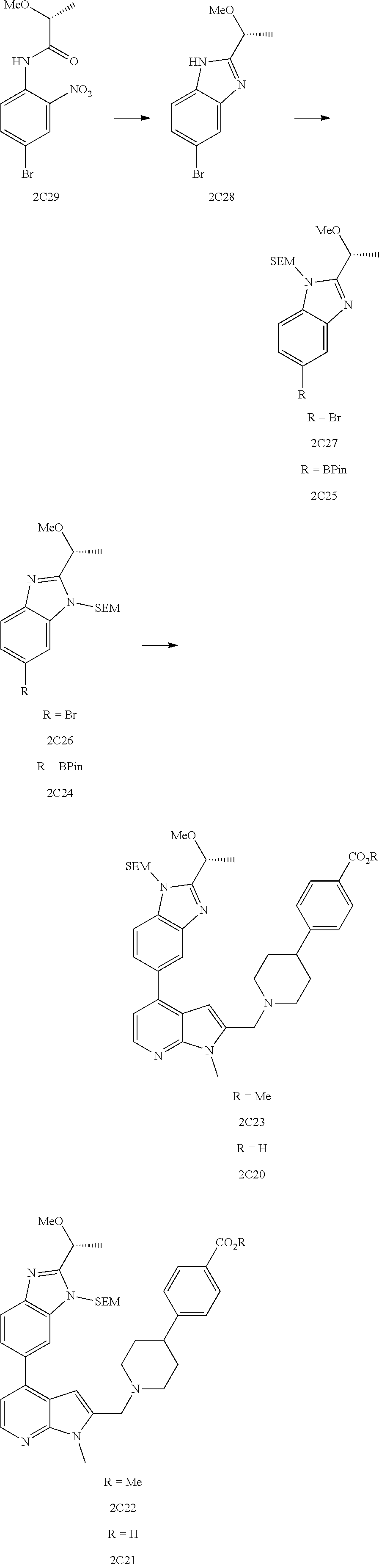

(R)-2-Methoxypropanoic acid (2.1 g) was stirred 1 h in pyridine/POCl₃ (15.4:1, 42.6 mL) at 0° C. 4-Bromo-2-nitroaniline (4.0 g) was added and stirring continued at 0° C. to RT ON. The mixture was diluted with aq. HCl to precipitate Int. 2C29 (R)—N-(4-bromo-2-nitro-phenyl)-2-methoxy-propanamide (4.0 g g). This material and iron powder (2.2 g) were stirred 4 h at 65° C. in EtOH: AcOH (1:1.75, 55 mL) and filtered. The OL (EtOAc/water) was dried, concentrated, and was washed with Et₂O and dried to give Int. 2C28 5-bromo-2-[(R)-1-methoxyethyl]-1H-benzimidazole (2.5 g). This material and NaH (60% oil dispersion; 0.35 g) were stirred 0.5 h in DMF (5 mL) at 0° C. SEM-Cl (2.0 g) was added and stirring continued 3 h at 0° C. to RT. The OL (EtOAc/water) was dried, concentrated, and purified by FC (hexane/EtOAc 4:1) to give Ints. 2C26/2C27 (R)-6-bromo-2-(1-methoxyethyl)-1-((2-(trimethylsilyl)-ethoxy)methyl)-1H-benzo[d]-imidazole and (R)-5-bromo-2-(1-methoxy-ethyl)-1-((2-(trimethyl-silyl) ethoxy)-methyl)-1H-benzo[d]-imidazole (2.0 g). 1.5 g of this mixture, PdDPPFCl₂-DCM (0.32 g), B₂Pin₂ (2.0 g), and KOAc (1.1 g) were degassed in dioxane (5 mL) and stirred ON at 100° C. The OL (EtOAc/water) was dried, concentrated, and purified by FC (hexane/EtOAc 55:45) to give Ints. 2C25/2C24 (R)-2-(1-methoxyethyl)-5-(4,4,5,5-tetramethyl-1,3,2-dioxa-borolan-2-yl)-1-((2-(trimethylsilyl)-ethoxy)methyl)-1H-benzo[d]imidazole and (R)-2-(1-methoxy-ethyl)-6-(4,4,5,5-tetramethyl-1,3,2-dioxaborolan-2-yl)-1-((2-(trimethylsilyl) ethoxy)-methyl)-1H-benzo[d]-imidazole (1.4 g). 0.24 g of this mixture, Int. 1AC5 (0.25 g), PdDPPFCl₂-DCM (45 mg), and NaHCO₃ (92 mg) were degassed in dioxane/water (6:1, 3.5 mL) and stirred ON at 100° C. The OL (EtOAc/water) was dried, concentrated, and purified by FC (pentane/EtOAc 4:1 to 7:3) to give Ints. 2C22/2C23 methyl (R)-4-(1-((4-(2-(1-methoxy-ethyl)-1-((2-(trimethylsilyl) ethoxy)methyl)-1H-benzo[d]-imidazol-5-yl)-1-methyl-1H-pyrrolo[2,3-b]pyridin-2-yl)methyl) piperidin-4-yl)benzoate and methyl (R)-4-(1-((4-(2-(1-methoxyethyl)-1-((2-(trimethylsilyl)-ethoxy)-methyl)-1H-benzo[d]imidazol-6-yl)-1-methyl-1H-pyrrolo[2,3-b]pyridin-2-yl)methyl)-piperidin-4-yl)-benzoate (0.2 g). This mixture and LiOH—H₂O (37 mg) were stirred ON in THF/MeOH/water (2:2:1, 2.5 mL) and concentrated. The residue was triturated in aq. citric acid to give Ints. 2C20/2C21 (R)-4-(1-((4-(2-(1-methoxyethyl)-1-((2-(trimethylsilyl) ethoxy)methyl)-1H-benzo[d]-imidazol-5-yl)-1-methyl-1H-pyrrolo[2,3-b]pyridin-2-yl)methyl) piperidin-4-yl)benzoic acid and (R)-4-(1-((4-(2-(1-methoxy-ethyl)-1-((2-(trimethylsilyl) ethoxy)methyl)-1H-benzo[d]imidazol-6-yl)-1-methyl-1H-pyrrolo[2,3-b]pyridin-2-yl)-methyl)piperidin-4-yl)benzoic acid (0.19 g).

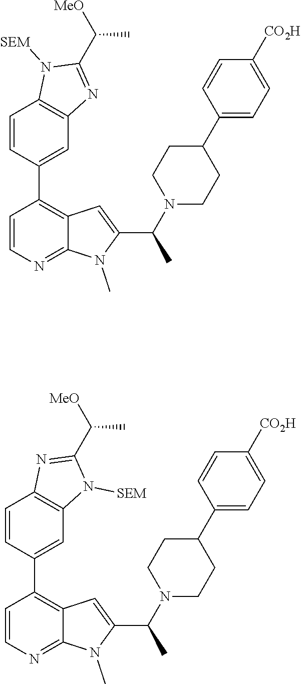 [SMILES: CO[C@H](C)c1nc2cc(-c3ccnc4c3cc([C@H](C)N3CCC(c5ccc(C(=O)O)cc5)CC3)n4C)ccc2n1COCC[Si](C)(C)C ; CO[C@H](C)c1nc2ccc(-c3ccnc4c3cc([C@H](C)N3CCC(c5ccc(C(=O)O)cc5)CC3)n4C)cc2n1COCC[Si](C)(C)C]

Int. 2C34/2C35 4-(1-((S)-1-(4-(2-((R)-1-methoxyethyl)-1-((2-(trimethylsilyl) ethoxy)methyl)-1H-benzo[d]imidazol-5-yl)-1-methyl-1H-pyrrolo[2,3-b]pyridin-2-yl)ethyl)piperidin-4-yl)benzoic acid and 4-(1-((S)-1-(4-(2-((R)-1-methoxyethyl)-1-((2-(trimethylsilyl) ethoxy)methyl)-1H-benzo[d]imidazol-6-yl)-1-methyl-1H-pyrrolo[2,3-b]pyridin-2-yl)ethyl)piperidin-4-yl)benzoic acid were prepared similarly to Ints. 2C20/2C21 from(S)-2-methoxypropanoic acid.

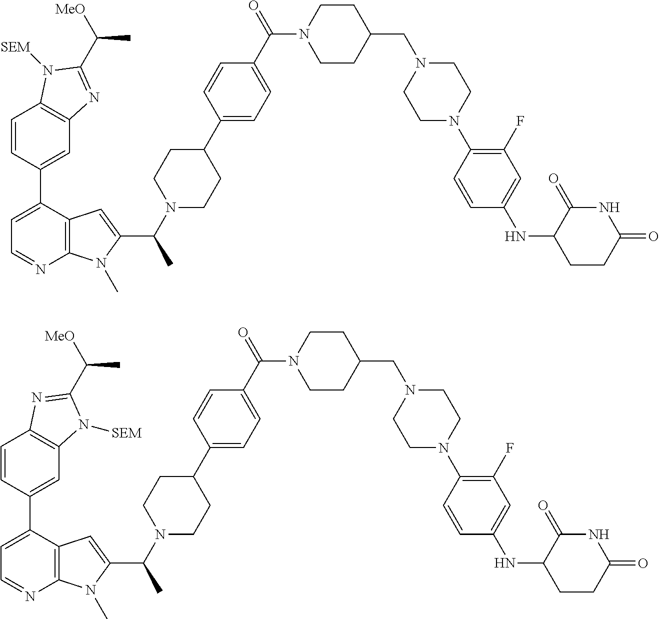 [SMILES: CO[C@@H](C)c1nc2cc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)ccc2n1COCC[Si](C)(C)C ; CO[C@@H](C)c1nc2ccc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)cc2n1COCC[Si](C)(C)C]

Int. 2C30 and 2C31. 3-((3-fluoro-4-(4-((1-(4-(1-((S)-1-(4-(2-((S)-1-methoxyethyl)-1-((2-(trimethylsilyl)-ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperazin-1-yl)phenyl)amino)piperidine-2,6-dione and 3-((3-fluoro-4-(4-((1-(4-(1-((S)-1-(4-(2-((S)-1-methoxyethyl)-1-((2-(trimethylsilyl) ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)-piperidine-2,6-dione (0.30 g) were prepared similarly to Int. 2C18 from Ints. 2C20/2C21/2C15 (0.20 g/0.29 g, HCl salt).

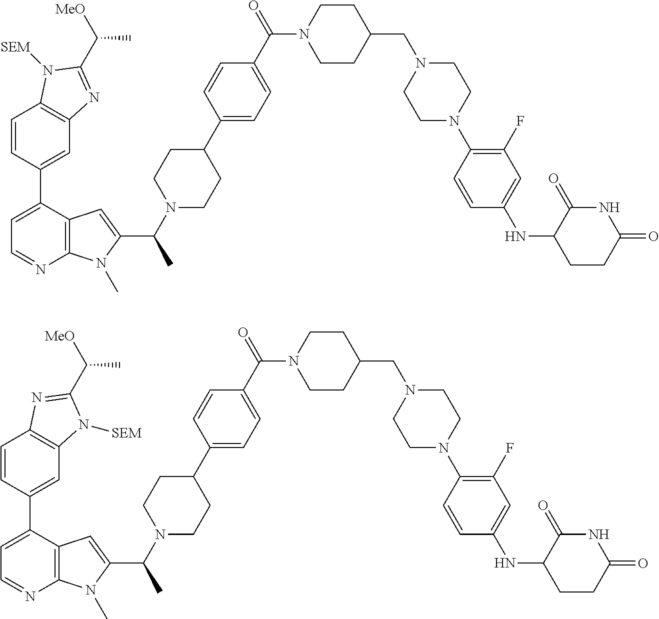 [SMILES: CO[C@H](C)c1nc2cc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)ccc2n1COCC[Si](C)(C)C ; CO[C@H](C)c1nc2ccc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)cc2n1COCC[Si](C)(C)C]

Int. 2C32 and 2C33. 3-((3-fluoro-4-(4-((1-(4-(1-((S)-1-(4-(2-((R)-1-methoxyethyl)-1-((2-(trimethylsilyl)-ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperazin-1-yl)phenyl)amino)piperidine-2,6-dione and 3-((3-fluoro-4-(4-((1-(4-(1-((S)-1-(4-(2-((R)-1-methoxyethyl)-1-((2-(trimethylsilyl) ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)-piperidine-2,6-dione (0.15 g) were prepared similarly to Int. 2C18 from Int. 2C34/2C35/2C15 (0.19 g/0.22 g, HCl salt).

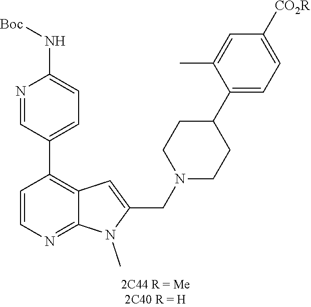

Methyl 4-bromo-3-methylbenzoate (2.5 g), tert-butyl 4-(4,4,5,5-tetramethyl-1,3,2-dioxaborolan-2-yl)-3,6-dihydropyridine-1(2H)-carboxylate (4.0 g), NaHCO₃ (2.8 g), and PdDPPFCl₂-DCM (0.45 g) were degassed in dioxane/water (10:1, 30 mL) and stirred ON at 100° C. The mixture was diluted with EtOAc (100 mL), filtered, concentrated, and purified by FC (hexane/EtOAc 9:1 to 85:15) to give Int. 2C41 tert-butyl 4-(4-methoxy-carbonyl-2-methyl-phenyl)-3,6-dihydro-2H-pyridine-1-carboxylate (3.2 g). Int. 2C41 (0.25 g) was stirred ON in DCM/4M HCl in dioxane (4:1, 10 mL) and concentrated. The residue was triturated in pentane to give Int. 2C42 methyl 3-methyl-4-(1,2,3,6-tetrahydropyridin-4-yl)benzoate (0.19 g, HCl salt). Ints. 1AB8/2C42 (1.2 g/1.2 g, HCl salt) were stirred 4 h in DCE/DIPEA (2:2:1, 7.3 mL) at 0° C. to RT. STAB (1.9 g) was added and stirring continued ON. The OL (EtOAc/water) was dried, concentrated, and purified by FC (hexane/EtOAc 7:3) to give Int. 2C43 methyl 4-(1-((4-bromo-1-methyl-1H-pyrrolo[2,3-b]pyridin-2-yl)methyl)-1,2,3,6-tetrahydropyridin-4-yl)-3-methylbenzoate (1.0 g). This material, tert-butyl (5-(4,4,5,5-tetramethyl-1,3,2-dioxaborolan-2-yl)pyridin-2-yl) carbamate (0.78 g), NaHCO₃ (0.28 g), and PdDPPFCl₂-DCM (90 mg) were degassed in dioxane/water (9:1, 8 mL) and stirred ON at 90° C. The OL (EtOAc/water) was concentrated and purified by FC (pentane/EtOAc 7:3) to give Int. 2C44 methyl 4-[1-[[4-[6-(tert-butoxycarbonylamino)-3-pyridyl]-1-methyl-pyrrolo[2,3-b]pyridin-2-yl]-methyl]-3,6-dihydro-2H-pyridin-4-yl]-3-methyl-benzoate (0.46 g). This material and LiOH—H2O (0.17 g) were stirred ON in THF/MeOH/water (6:2:1, 7 mL). The mixture was neutralized with 0.5M aq. HCl to precipitate Int. 2C40 4-(1-((4-(6-((tert-butoxycarbonyl)amino)pyridin-3-yl)-1-methyl-1H-pyrrolo[2,3-b]pyridin-2-yl)methyl)piperidin-4-yl)-3-methylbenzoic acid (0.18 g).

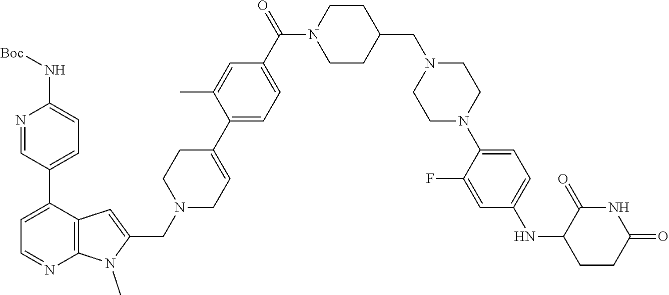 [SMILES: Cc1cc(C(=O)N2CCC(CN3CCN(c4ccc(NC5CCC(=O)NC5=O)cc4F)CC3)CC2)ccc1C1=CCN(Cc2cc3c(-c4ccc(NC(=O)OC(C)(C)C)nc4)ccnc3n2C)CC1]

Int. 2C45. tert-butyl (5-(2-((4-(4-(4-((4-(4-((2,6-dioxopiperidin-3-yl)amino)-2-fluorophenyl)-piperazin-1-yl)methyl)piperidine-1-carbonyl)-2-methylphenyl)-3,6-dihydropyridin-1(2H)-yl)methyl)-1-methyl-1H-pyrrolo[2,3-b]pyridin-4-yl)pyridin-2-yl) carbamate (27 mg) was prepared similarly to Int. 2C18 from Ints. 2C40/2C15 (25 mg/20 mg, HCl salt).

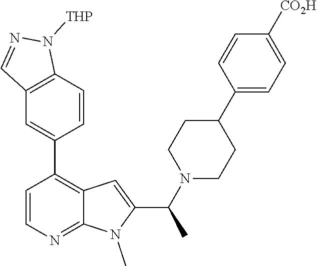

Int. 3E39 (59 mg) was stirred ON in MeOH/THF/1M aq. NaOH (2.6:1:3:1, 2 mL) at 0° C. to RT. The mixture was partially concentrated. The OL (brine and 1M aq. HCl/DCM/MeOH (9:1)) was dried, concentrated, and purified by FC (DCM/MeOH 95:5) to give Int. 2C54 4-(1-((1S)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoic acid (47 mg).

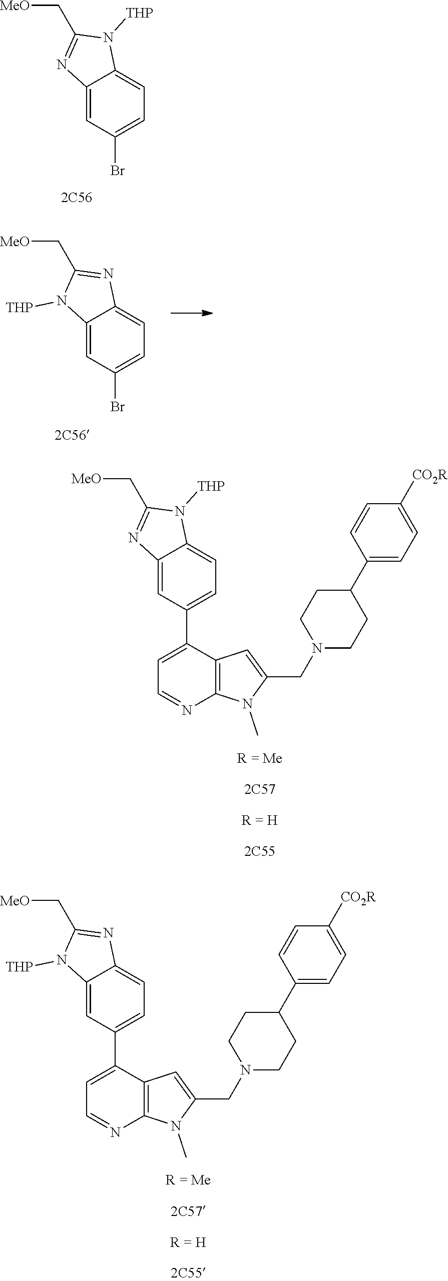

5-Bromo-2-(methoxymethyl)-1H-benzo[d]imidazole (1.0 g) and TsOH-H₂O (0.16 g) were stirred ON in THF/3,4-dihydropyran (4:1, 12.3 mL) at 80° C. The OL (EtOAc/water) was washed with brine, dried, concentrated, and purified by FC (pentane/EtOAc 5:1 to 4:1) to give Ints. 2C56/2C56′ 5-bromo-2-(methoxymethyl)-1-(tetrahydro-2H-pyran-2-yl)-1H-benzo[d]imidazole/6-bromo-2-(methoxymethyl)-1-(tetrahydro-2H-pyran-2-yl)-1H-benzo[d]imidazole (0.50 g). Ints. 2C56/2C56′/ 3E170 (0.40 g/0.72 g), NaHCO₃ (0.31 g), and Pd(PPh₃)₄ (50 mg) were degassed in dioxane/water (4:1, 5 mL) and stirred ON at 90° C. Filtration, concentration, and purification by FC (hexane/EtOAc 1:1) gave Ints. 2C57/2C57′ methyl 4-(1-((4-(2-(methoxymethyl)-1-(tetrahydro-2H-pyran-2-yl)-1H-benzo[d]imidazol-5-yl)-1-methyl-1H-pyrrolo[2,3-b]pyridin-2-yl)methyl)piperidin-4-yl)benzoate/methyl 4-(1-((4-(2-(methoxymethyl)-1-(tetrahydro-2H-pyran-2-yl)-1H-benzo[d]imidazol-6-yl)-1-methyl-1H-pyrrolo[2,3-b]pyridin-2-yl)methyl)piperidin-4-yl)benzoate (70 mg). Int. 2C57 (1.0 g) and LiOH—H₂O (0.35 g) were stirred ON in THF/MeOH/water (3:3:1, 5 mL) at 0° C. to RT and concentrated. The residue was triturated with dilute aq. HCl to give Ints. 2C55/2C55′ 4-(1-((4-(2-(methoxymethyl)-1-(tetrahydro-2H-pyran-2-yl)-1H-benzo[d] imidazol-5-yl)-1-methyl-1H-pyrrolo[2,3-b]pyridin-2-yl)methyl)piperidin-4-yl)benzoic acid/4-(1-((4-(2-(methoxymethyl)-1-(tetrahydro-2H-pyran-2-yl)-1H-benzo[d]imidazol-6-yl)-1-methyl-1H-pyrrolo[2,3-b]pyridin-2-yl)methyl)piperidin-4-yl)benzoic acid (0.58 g, HCl salt).

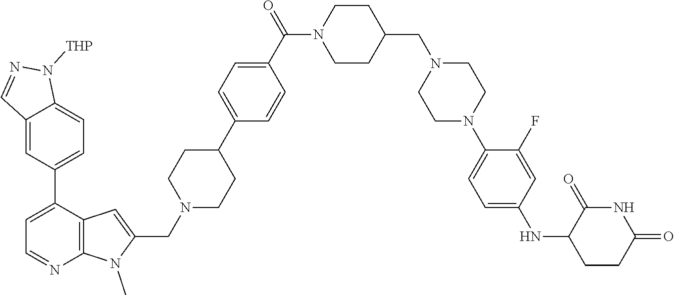

HCl salts of Ints. 3A2/2C60 (0.31 g/0.25 g) and HATU (0.22 g) were stirred ON in DMF/DIPEA (15:1, 6.4 mL) at 0° C. to RT. The mixture was diluted with water to precipitate a solid that was purified by FC (DCM/MeOH 9:1) to give Int. 2C64 3-((3-fluoro-4-(4-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)piperazin-1-yl)phenyl)amino)piperidine-2,6-dione (0.35 g).

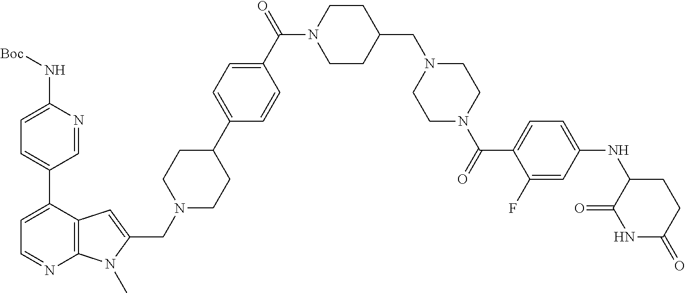 [SMILES: Cn1c(CN2CCC(c3ccc(C(=O)N4CCC(CN5CCN(C(=O)c6ccc(NC7CCC(=O)NC7=O)cc6F)CC5)CC4)cc3)CC2)cc2c(-c3ccc(NC(=O)OC(C)(C)C)nc3)ccnc21]

Int. 2E1. tert-butyl (5-(2-((4-(4-(4-((4-(4-((2,6-dioxopiperidin-3-yl)amino)-2-fluorobenzoyl) piperazin-1-yl)methyl)piperidine-1-carbonyl)phenyl) piperidin-1-yl)methyl)-1-methyl-1H-pyrrolo[2,3-b]pyridin-4-yl)pyridin-2-yl) carbamate (33 mg) was prepared similarly to Int. 2C18 from Ints. 2E6/2E2 (25 mg/32 mg, TFA salt).

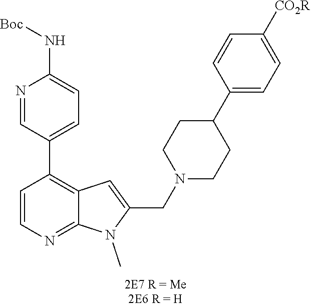

PdDPPFCl₂-DCM (0.37 g), Int. 1AC5 (2.0 g), tert-butyl (5-(4,4,5,5-tetramethyl-1,3,2-dioxaborolan-2-yl)pyridin-2-yl) carbamate (1.6 g), and NaHCO₃ (0.76 g), were degassed in dioxane/water (10:2, 22 mL) and stirred ON at 80° C. The OL (water/EtOAc) was dried, concentrated, and purified by FC (hexane/EtOAc 3:7) to give Int. 2E7 methyl 4-(1-((4-(6-((tert-butoxycarbonyl)amino)pyridin-3-yl)-1-methyl-1H-pyrrolo[2,3-b]pyridin-2-yl)-methyl)-piperidin-4-yl)benzoate (2.1 g). This material and LiOH—H₂O (0.93 g) were stirred 5 h in water/MeOH/THF (1:2:2, 10 mL) at 0° C. and concentrated. The residue was triturated in dilute aq. HCl to give Int. 2E6 4-(1-((4-(6-((tert-butoxycarbonyl)amino)-pyridin-3-yl)-1-methyl-1H-pyrrolo[2,3-b]pyridin-2-yl)methyl)piperidin-4-yl)benzoic acid (1.7 g).

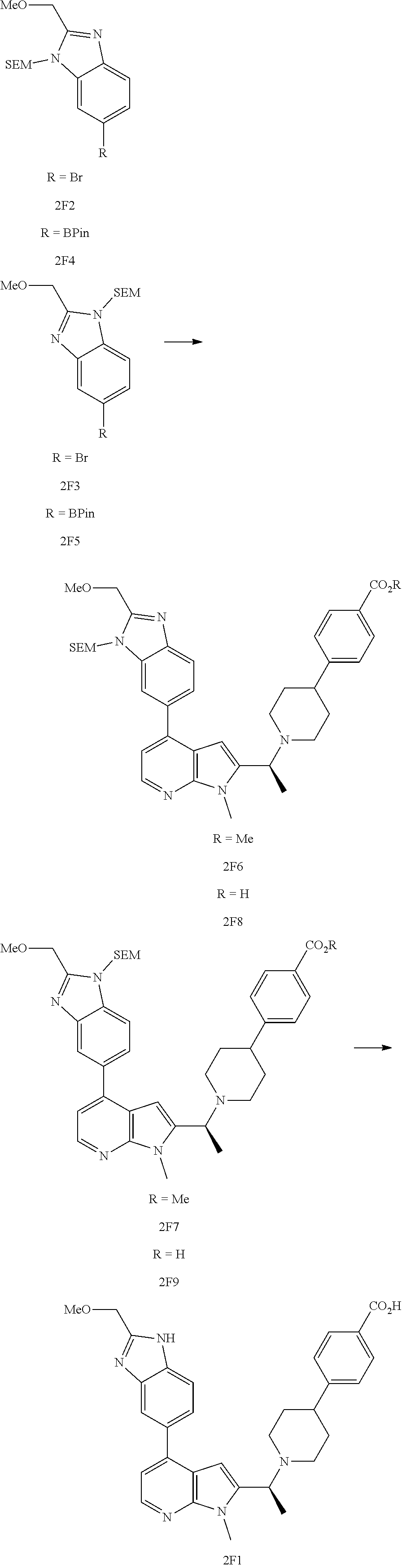

5-Bromo-2-(methoxymethyl)-1H-benzo[d]imidazole (25 g) was added to a mixture of NaH (60% oil dispersion, 6.22 g) in DMF (150 mL) at 0° C. SEM-Cl (20.8 g) was added after 0.25 h at 0° C. The OL (water/EtOAc) was dried and concentrated. This material was mixed with another batch prepared similarly on 10 g scale and purified by FC (pentane/EtOAc 3:7) to give Ints. 2F2/2F3 6-bromo-2-(methoxy-methyl)-1-((2-(trimethylsilyl) ethoxy)methyl)-1H-benzo[d]-imidazole and 5-bromo-2-(methoxy-methyl)-1-((2-(trimethylsilyl)-ethoxy)methyl)-1H-benzo[d]-imidazole (30 g). 3.0 g of this mixture, B₂Pin₂ (2.25 g), PdDPPFCl₂-DCM (0.33 g), and KOAc (2.38 g) degassed in dioxane (25 mL) and stirred ON at 90° C. The filtrate after filtration was concentrated and purified by FC (pentane/EtOAc 1:4) to give Ints. 2F4/2F5 2-(methoxy-methyl)-6-(4,4,5,5-tetramethyl-1,3,2-dioxaborolan-2-yl)-1-((2-(trimethylsilyl) ethoxy)methyl)-1H-benzo[d]-imidazole and 2-(methoxymethyl)-5-(4,4,5,5-tetramethyl-1,3,2-dioxaborolan-2-yl)-1-((2-(trimethyl-silyl) ethoxy)-methyl)-1H-benzo[d]imidazole (2.1 g). 0.50 g of this mixture, Int. 3E35 (0.50 g), PdDPPFCl₂-DCM (89 mg), and NaHCO₃were degassed in dioxane/water (3:1, 20 mL) and stirred ON at 80° C. and filtered. The OL (EtOAc/water) was dried and concentrated. This material was mixed with another batch prepared similarly from Int. 3E35 (2.5 g) and purified by FC (heptane/EtOAc 7:3 to 2:3) to give Ints. 2F6/2F7 methyl(S)-4-(1-(1-(4-(2-(methoxy-methyl)-1-((2-(trimethylsilyl)-ethoxy)-methyl)-1H-benzo[d]-imidazol-6-yl)-1-methyl-1H-pyrrolo [2,3-b] pyridin-2-yl)ethyl)-piperidin-4-yl)-benzoate and methyl(S)-4-(1-(1-(4-(2-(methoxymethyl)-1-((2-(trimethyl-silyl) ethoxy)-methyl)-1H-benzo[d]-imidazol-5-yl)-1-methyl-1H-pyrrolo[2,3-b]pyridin-2-yl)ethyl)piperidin-4-yl)-benzoate (3.2 g). 3.0 g of this mixture and LiOH—H₂O (0.55 g) were stirred ON at 0° C. to RT in THF/MeOH/water (2:2:1, 25 mL). The mixture was partially concentrated and triturated in dilute aq. HCl to give Ints. 2F8/2F9 (S)-4-(1-(1-(4-(2-(methoxy-methyl)-1-((2-(trimethyl-silyl)-ethoxy)methyl)-1H-benzo[d]imidazol-6-yl)-1-methyl-1H-pyrrolo[2,3-b]pyridin-2-yl)ethyl)piperidin-4-yl)benzoic acid and(S)-4-(1-(1-(4-(2-(methoxy-methyl)-1-((2-(trimethylsilyl)-ethoxy)-methyl)-1H-benzo[d]-imidazol-5-yl)-1-methyl-1H-pyrrolo[2,3-b]pyridin-2-yl)ethyl)-piperidin-4-yl)benzoic acid (2.5 g). This material was stirred ON in DCM/4M HCl in dioxane (0.6:1, 40 mL) at 0° C. to RT, concentrated, and triturated in Et₂O to give Int. 2F1 4-[1-[(1S)-1-[4-[2-(methoxymethyl)-1H-benzimidazol-5-yl]-1-methyl-pyrrolo[2,3-b]pyridin-2-yl]ethyl]-4-piperidyl]-benzoic acid (2.0 g, HCl salt).

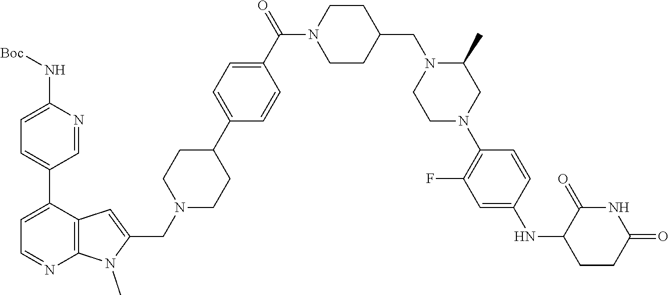 [SMILES: C[C@H]1CN(c2ccc(NC3CCC(=O)NC3=O)cc2F)CCN1CC1CCN(C(=O)c2ccc(C3CCN(Cc4cc5c(-c6ccc(NC(=O)OC(C)(C)C)nc6)ccnc5n4C)CC3)cc2)CC1]

Int. 2F20. tert-butyl (5-(2-((4-(4-(4-(((2S)-4-(4-((2,6-dioxopiperidin-3-yl)amino)-2-fluorophenyl)-2-methylpiperazin-1-yl)methyl)piperidine-1-carbonyl)phenyl) piperidin-1-yl)methyl)-1-methyl-1H-pyrrolo[2,3-b]pyridin-4-yl)pyridin-2-yl) carbamate (50 mg) was prepared similarly to Int. 2C18 from HCl salts of Ints. 2E6/2F21 (48 mg/34 mg).

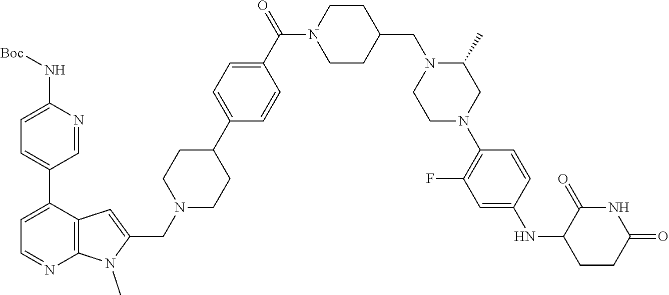 [SMILES: C[C@@H]1CN(c2ccc(NC3CCC(=O)NC3=O)cc2F)CCN1CC1CCN(C(=O)c2ccc(C3CCN(Cc4cc5c(-c6ccc(NC(=O)OC(C)(C)C)nc6)ccnc5n4C)CC3)cc2)CC1]

Int. 2F27. tert-butyl (5-(2-((4-(4-(4-(((2R)-4-(4-((2,6-dioxopiperidin-3-yl)amino)-2-fluorophenyl)-2-methylpiperazin-1-yl)methyl)piperidine-1-carbonyl)phenyl) piperidin-1-yl)methyl)-1-methyl-1H-pyrrolo[2,3-b]pyridin-4-yl)pyridin-2-yl) carbamate (39 mg) was prepared similarly to Int. 2C18 from Ints. 2E6/2F21 (34 mg/HCl salt).

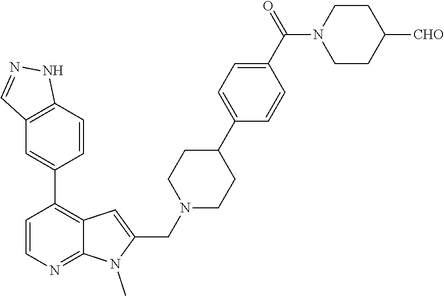 [SMILES: Cn1c(CN2CCC(c3ccc(C(=O)N4CCC(C=O)CC4)cc3)CC2)cc2c(-c3ccc4[nH]ncc4c3)ccnc21]

A solution of Int. 3D4 (0.10 g) in DCM/TFA (2:1, 7.5 mL) was stirred 1 h and concentrated to give Int. 2G1 1-(4-(1-((4-(1H-indazol-5-yl)-1-methyl-1H-pyrrolo[2,3-b]pyridin-2-yl)methyl)piperidin-4-yl)benzoyl)piperidine-4-carbaldehyde (97 mg, TFA salt).

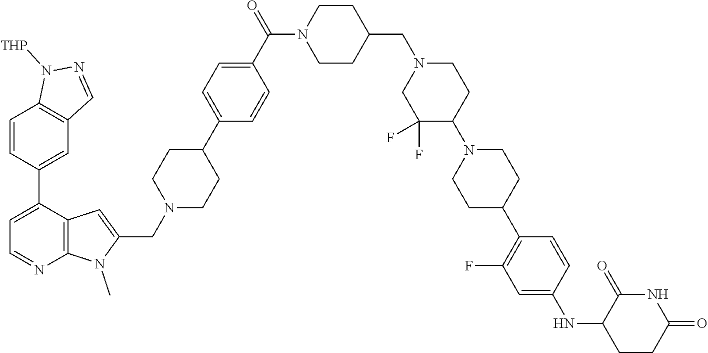

Int. 2H1. 3-((4-(3′, 3′-difluoro-1′-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)-[1,4′-bipiperidin]-4-yl)-3-fluorophenyl)amino)piperidine-2,6-dione (22 mg) was prepared similarly to Int. 2C18 from HCl salts of Ints. 3A2/2H2 (24 mg/22 mg).

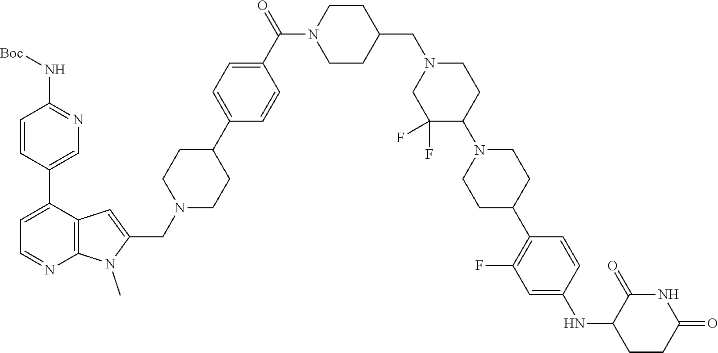 [SMILES: Cn1c(CN2CCC(c3ccc(C(=O)N4CCC(CN5CCC(N6CCC(c7ccc(NC8CCC(=O)NC8=O)cc7F)CC6)C(F)(F)C5)CC4)cc3)CC2)cc2c(-c3ccc(NC(=O)OC(C)(C)C)nc3)ccnc21]

Int. 2H11. tert-butyl (5-(2-((4-(4-(4-((4-(4-((2,6-dioxopiperidin-3-yl)amino)-2-fluorophenyl)-3′, 3′-difluoro-[1,4′-bipiperidin]-1′-yl)methyl)piperidine-1-carbonyl)phenyl) piperidin-1-yl)methyl)-1-methyl-1H-pyrrolo[2,3-b]pyridin-4-yl)pyridin-2-yl) carbamate (33 mg) was prepared similarly to Int. 2C18 from HCl salts of Ints. 2E6/2H2 (20 mg/24 mg).

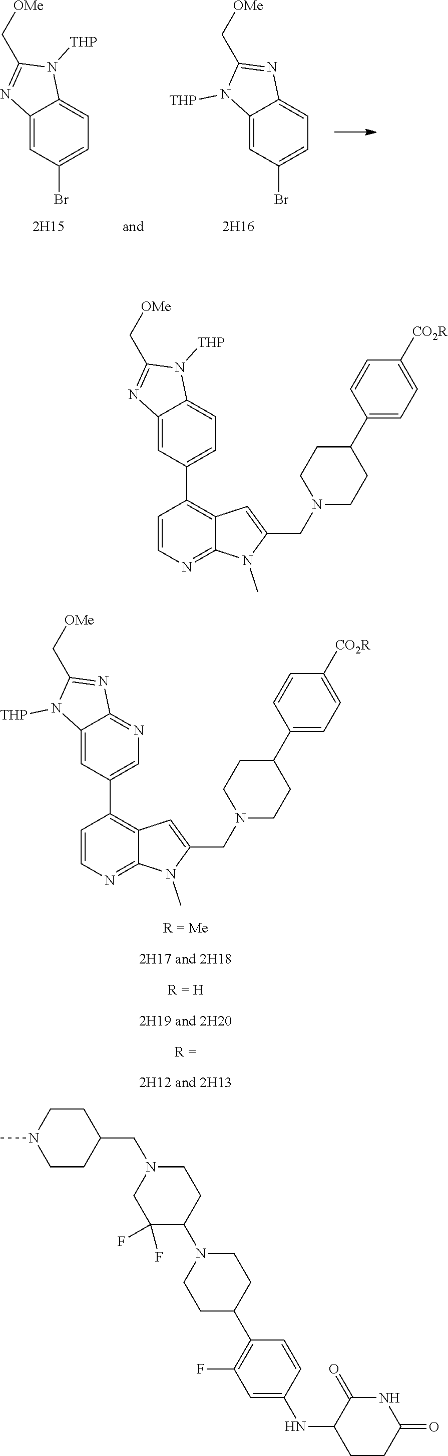

Int. 1AC5 (3.0 g), B₂Pin₂ (1.1 g), KOAc (2.0 g), and PdDPPFCl₂-DCM (50 mg) were degassed in dioxane (30 mL) and stirred ON at 90° C. The filtrate after filtration was concentrated and purified by FC (pentane/EtOAc 1:4) to give Int. 2H14 methyl 4-[1-[1-methyl-4-(4,4,5,5-tetramethyl-1,3,2-dioxaborolan-2-yl)-pyrrolo[2,3-b]pyridin-2-yl]methyl]-4-piperidyl]benzoate (1.7 g). 5-Bromo-2-(methoxymethyl)-1H-benzo[d]imidazole (10 g), and TsOH-H₂O (1.6 g) were stirred ON at 80° C. in THF/3,4-dihydropyran (4:1, 125 mL). The OL (EtOAc/water) was washed with brine, dried, concentrated, and purified by FC (pentane/EtOAc 4:1 to 3:1) to give Ints. 2H15/2H16 5-bromo-2-(methoxymethyl)-1-(tetrahydro-2H-pyran-2-yl)-1H-benzo[d]imidazole and 6-bromo-2-(methoxy-methyl)-1-(tetrahydro-2H-pyran-2-yl)-1H-benzo[d]-imidazole (5.0 g). Ints. 2H14/2H15/2H16 (4.52 g/2.0 g), NaHCO₃ (1.55 g), and Pd(PPh₃)₄ (0.36 g) were degassed in dioxane/water (5:1, 24 mL) and stirred ON at 90° C., filtered, concentrated, and purified by FC (hexane/EtOAc 1:1) to give Ints. 2H17/2H18 methyl 4-(1-((4-(2-(methoxy-methyl)-1-(tetrahydro-2H-pyran-2-yl)-1H-benzo[d]imidazol-6-yl)-1-methyl-1H-pyrrolo[2,3-b]-pyridin-2-yl)-methyl)piperidin-4-yl)benzoate and methyl 4-(1-((4-(2-(methoxymethyl)-1-(tetra-hydro-2H-pyran-2-yl)-1H-benzo[d]imidazol-5-yl)-1-methyl-1H-pyrrolo [2,3-b] pyridin-2-yl)methyl)-piperidin-4-yl)benzoate (1.1 g). Ints. 2H17/2H18 (1.0 g) and LiOH—H₂O (0.35 g) were stirred 8 h in THF/MeOH/water (3:3:1; 5 mL) at 0° C. to RT, concentrated, and triturated in dilute aq. HCl to give Ints. 2H19/2H₂O 4-(1-((4-(2-(methoxy-methyl)-1-(tetra-hydro-2H-pyran-2-yl)-1H-benzo[d] imidazol-5-yl)-1-methyl-1H-pyrrolo[2,3-b]pyridin-2-yl)-methyl)-piperidin-4-yl)benzoic acid and 4-(1-((4-(2-(methoxy-methyl)-1-(tetra-hydro-2H-pyran-2-yl)-1H-benzo[d]-imidazol-6-yl)-1-methyl-1H-pyrrolo[2,3-b]pyridin-2-yl)methyl)-piperidin-4-yl)benzoic acid (0.58 g, HCl salt). 18 mg of this mixture and Int. 2H2 (21 mg, HCl salt) were converted to Ints. 2H12/2H13 3-((4-(3′, 3′-difluoro-1′-((1-(4-(1-((4-(2-(methoxymethyl)-1-(tetrahydro-2H-pyran-2-yl)-1H-benzo[d]imidazol-5-yl)-1-methyl-1H-pyrrolo[2,3-b]pyridin-2-yl)methyl)piperidin-4-yl)benzoyl)-piperidin-4-yl)-methyl)-[1,4′-bipiperidin]-4-yl)-3-fluorophenyl)amino)piperidine-2,6-dione and 3-((4-(3′, 3′-difluoro-1′-((1-(4-(1-((4-(2-(methoxymethyl)-1-(tetrahydro-2H-pyran-2-yl)-1H-benzo[d]imidazol-6-yl)-1-methyl-1H-pyrrolo[2,3-b]-pyridin-2-yl)methyl)piperidin-4-yl)benzoyl)piperidin-4-yl)methyl)-[1,4′-bi-piperidin]-4-yl)-3-fluorophenyl)-amino)piperidine-2,6-dione (19 mg) similarly to Int. 2C18.

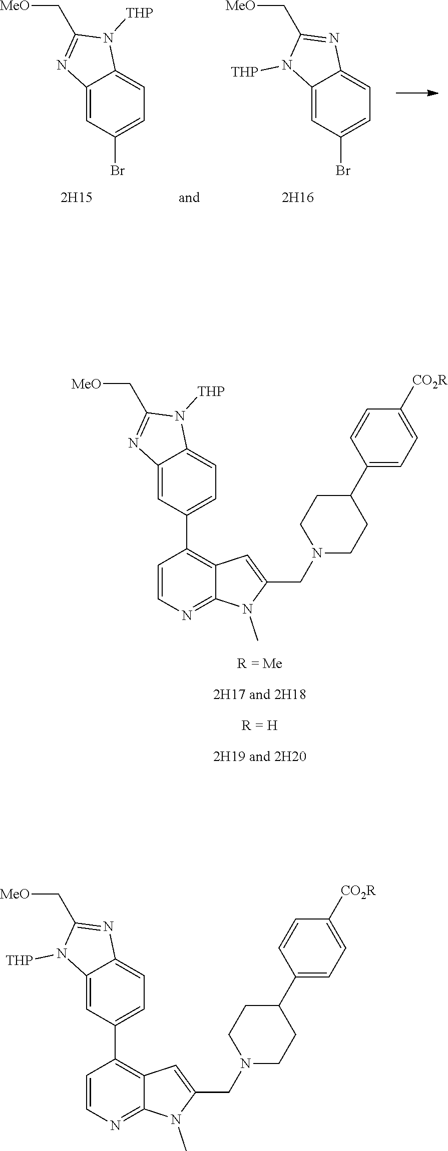

5-Bromo-2-(methoxymethyl)-1H-benzo[d]imidazole (10 g), and TsOH-H₂O (1.6 g) were stirred ON at 80° C. in THF/3,4-dihydropyran (4:1, 125 mL). The OL (EtOAc/water) was washed with brine, dried, concentrated, and purified by FC (pentane/EtOAc 4:1 to 3:1) to give Ints. 2H15/2H16 5-bromo-2-(methoxymethyl)-1-(tetrahydro-2H-pyran-2-yl)-1H-benzo[d]imidazole and 6-bromo-2-(methoxy-methyl)-1-(tetrahydro-2H-pyran-2-yl)-1H-benzo[d]-imidazole (5.0 g). Ints. 2H14/2H15/2H16 (4.52 g/2.0 g), NaHCO₃ (1.55 g), and Pd(PPh₃)₄ (0.36 g) were degassed in dioxane/water (5:1, 24 mL) and stirred ON at 90° C., filtered, concentrated, and purified by FC (hexane/EtOAc 1:1) to give Ints. 2H17/2H18 methyl 4-(1-((4-(2-(methoxy-methyl)-1-(tetrahydro-2H-pyran-2-yl)-1H-benzo[d]imidazol-6-yl)-1-methyl-1H-pyrrolo[2,3-b]-pyridin-2-yl)-methyl)piperidin-4-yl)benzoate and methyl 4-(1-((4-(2-(methoxymethyl)-1-(tetra-hydro-2H-pyran-2-yl)-1H-benzo[d]imidazol-5-yl)-1-methyl-1H-pyrrolo [2,3-b] pyridin-2-yl)methyl)-piperidin-4-yl)benzoate (1.1 g). Ints. 2H17/2H18 (1.0 g) and LiOH—H₂O (0.35 g) were stirred 8 h in THF/MeOH/water (3:3:1; 5 mL) at 0° C. to RT, concentrated, and triturated in dilute aq. HCl to give Ints. 2H19/2H₂O 4-(1-((4-(2-(methoxy-methyl)-1-(tetra-hydro-2H-pyran-2-yl)-1H-benzo[d] imidazol-5-yl)-1-methyl-1H-pyrrolo[2,3-b]pyridin-2-yl)-methyl)-piperidin-4-yl)benzoic acid and 4-(1-((4-(2-(methoxy-methyl)-1-(tetra-hydro-2H-pyran-2-yl)-1H-benzo[d]imidazol-6-yl)-1-methyl-1H-pyrrolo[2,3-b]pyridin-2-yl)methyl)-piperidin-4-yl)benzoic acid (0.58 g, HCl salt).

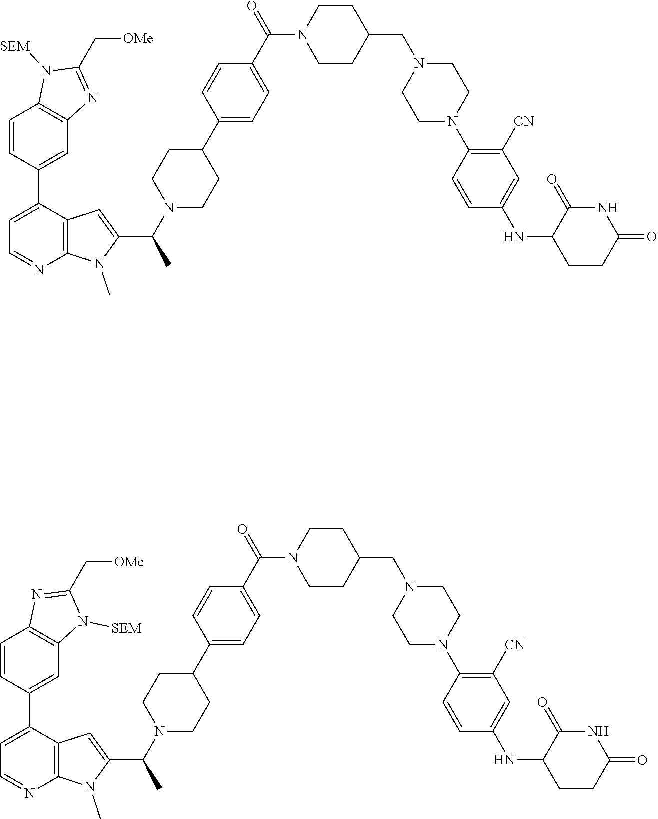 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8C#N)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ccc(NC9CCC(=O)NC9=O)cc8C#N)CC7)CC6)cc5)CC3)n4C)cc2n1COCC[Si](C)(C)C]

Int. 215 and 216. 5-((2,6-dioxopiperidin-3-yl)amino)-2-(4-((1-(4-(1-((S)-1-(4-(2-(methoxymethyl)-1-((2-(trimethyl-silyl) ethoxy)methyl)-1H-benzo[d]imidazol-5-yl)-1-methyl-1H-pyrrolo[2,3-b]pyridin-2-yl)ethyl)-piperidin-4-yl)-benzoyl)piperidin-4-yl)methyl)piperazin-1-yl)benzonitrile and 5-((2,6-dioxopiperidin-3-yl)amino)-2-(4-((1-(4-(1-((S)-1-(4-(2-(methoxymethyl)-1-((2-(trimethylsilyl) ethoxy)methyl)-1H-benzo[d]-imidazol-6-yl)-1-methyl-1H-pyrrolo[2,3-b]pyridin-2-yl)ethyl)piperidin-4-yl)benzoyl)piperidin-4-yl)-methyl)piperazin-1-yl)-benzonitrile (0.12 g) was prepared similarly to Int. 2C18 from Ints. 2F8/2F9/2I13 (0.10 g/96 mg, HCl salt).

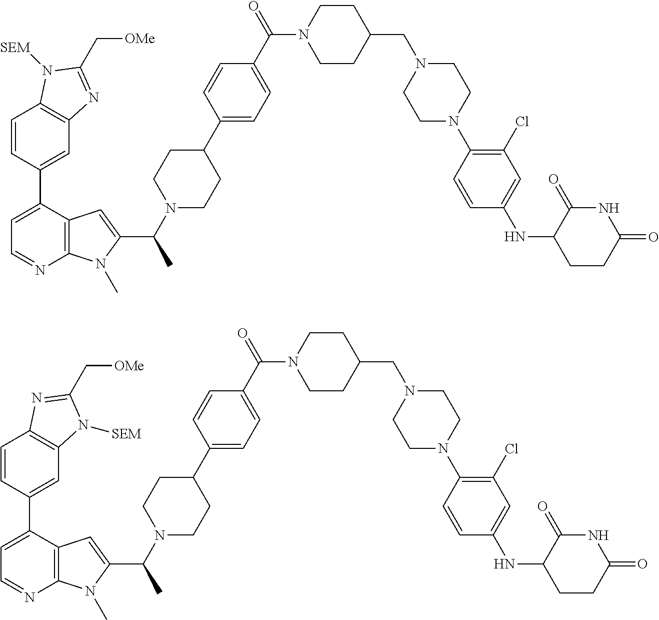 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8Cl)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ccc(NC9CCC(=O)NC9=O)cc8Cl)CC7)CC6)cc5)CC3)n4C)cc2n1COCC[Si](C)(C)C]

Int. 2I7 and 2I8. 3-((3-chloro-4-(4-((1-(4-(1-((S)-1-(4-(2-(methoxymethyl)-1-((2-(trimethylsilyl) ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)-methyl)piperazin-1-yl)phenyl)amino)piperidine-2,6-dione and 3-((3-chloro-4-(4-((1-(4-(1-((S)-1-(4-(2-(methoxy-methyl)-1-((2-(trimethylsilyl) ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione (0.17 g) was prepared similarly to Int. 2C18 from Ints. 2F8/2F9/2I9 (0.20 g/0.21 g, HCl salt).

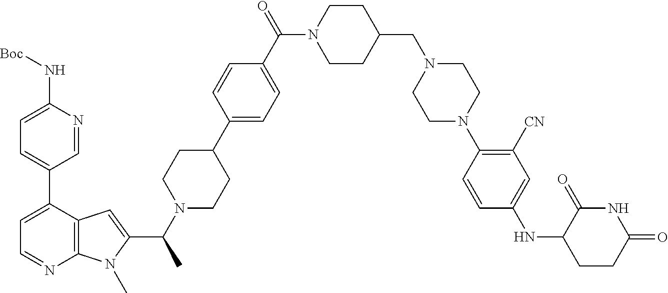 [SMILES: C[C@@H](c1cc2c(-c3ccc(NC(=O)OC(C)(C)C)nc3)ccnc2n1C)N1CCC(c2ccc(C(=O)N3CCC(CN4CCN(c5ccc(NC6CCC(=O)NC6=O)cc5C#N)CC4)CC3)cc2)CC1]

Int. 2I17. tert-butyl (5-(2-((1S)-1-(4-(4-(4-((4-(2-cyano-4-((2,6-dioxopiperidin-3-yl)amino)phenyl)-piperazin-1-yl)methyl)piperidine-1-carbonyl)phenyl) piperidin-1-yl)ethyl)-1-methyl-1H-pyrrolo[2,3-b]pyridin-4-yl)pyridin-2-yl) carbamate (0.15 g) was prepared similarly to Int. 2C18 from Ints. 3E76/2I13 (0.10 g/81 mg, HCl salt).

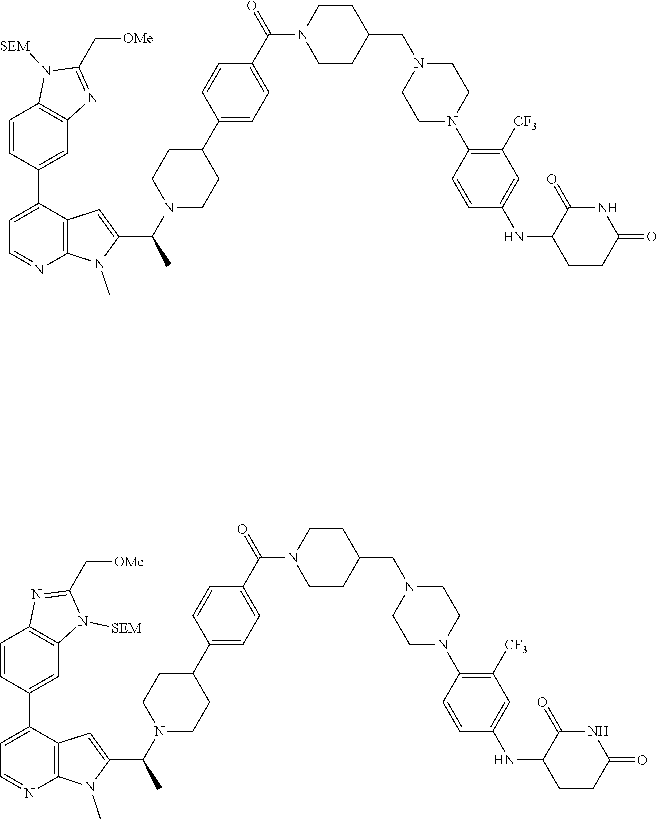 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8C(F)(F)F)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ccc(NC9CCC(=O)NC9=O)cc8C(F)(F)F)CC7)CC6)cc5)CC3)n4C)cc2n1COCC[Si](C)(C)C]

Int. 2I18 and 2I19. 3-((4-(4-((1-(4-(1-((S)-1-(4-(2-(methoxymethyl)-1-((2-(trimethylsilyl) ethoxy)methyl)-1H-benzo[d]imidazol-5-yl)-1-methyl-1H-pyrrolo[2,3-b]pyridin-2-yl)ethyl)piperidin-4-yl)benzoyl)piperidin-4-yl)-methyl)piperazin-1-yl)-3-(trifluoromethyl)phenyl)amino)piperidine-2,6-dione and 3-((4-(4-((1-(4-(1-((S)-1-(4-(2-(methoxymethyl)-1-((2-(trimethylsilyl) ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)-3-(trifluoromethyl)phenyl)-amino)piperidine-2,6-dione (0.50 g) was prepared similarly to Int. 2C18 from Ints. 2F8/2F9/2I20 (0.20 g/0.58 g, HCl salt).

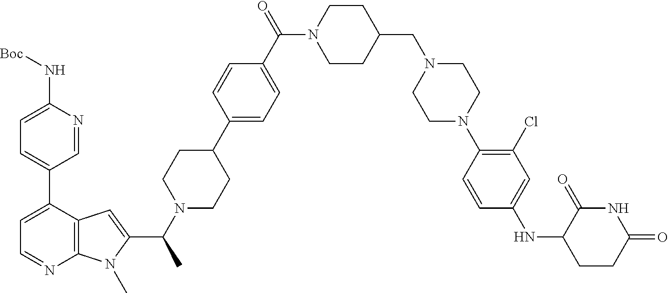 [SMILES: C[C@@H](c1cc2c(-c3ccc(NC(=O)OC(C)(C)C)nc3)ccnc2n1C)N1CCC(c2ccc(C(=O)N3CCC(CN4CCN(c5ccc(NC6CCC(=O)NC6=O)cc5Cl)CC4)CC3)cc2)CC1]

Int. 2I28. tert-butyl (5-(2-((1S)-1-(4-(4-(4-((4-(2-chloro-4-((2,6-dioxopiperidin-3-yl)amino)-phenyl) piperazin-1-yl)methyl)piperidine-1-carbonyl)phenyl) piperidin-1-yl)ethyl)-1-methyl-1H-pyrrolo[2,3-b]pyridin-4-yl)pyridin-2-yl) carbamate (60 mg) was prepared similarly to Int. 2C18 from Ints. 3E76/2I9 (0.10 g/0.16 g, HCl salt).

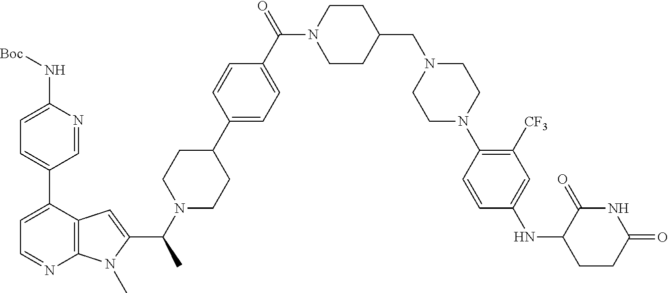 [SMILES: C[C@@H](c1cc2c(-c3ccc(NC(=O)OC(C)(C)C)nc3)ccnc2n1C)N1CCC(c2ccc(C(=O)N3CCC(CN4CCN(c5ccc(NC6CCC(=O)NC6=O)cc5C(F)(F)F)CC4)CC3)cc2)CC1]

Int. 2I29. tert-butyl (5-(2-((1S)-1-(4-(4-(4-((4-(4-((2,6-dioxopiperidin-3-yl)amino)-2-(trifluoromethyl)phenyl)-piperazin-1-yl)methyl)piperidine-1-carbonyl)phenyl) piperidin-1-yl)ethyl)-1-methyl-1H-pyrrolo[2,3-b]pyridin-4-yl)pyridin-2-yl) carbamate (0.25 g) was prepared similarly to Int. 2C18 from Ints. 3E76/2I20 (0.15 g/0.51 g, HCl salt).

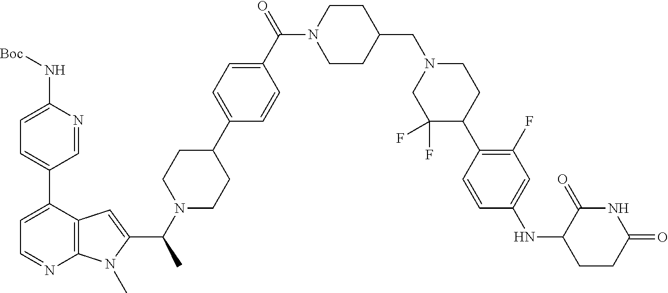 [SMILES: C[C@@H](c1cc2c(-c3ccc(NC(=O)OC(C)(C)C)nc3)ccnc2n1C)N1CCC(c2ccc(C(=O)N3CCC(CN4CCC(c5ccc(NC6CCC(=O)NC6=O)cc5F)C(F)(F)C4)CC3)cc2)CC1]

Int. 2M6. tert-butyl (5-(2-((1S)-1-(4-(4-(4-((4-(4-((2,6-dioxopiperidin-3-yl)amino)-2-fluorophenyl)-3,3-difluoropiperidin-1-yl)methyl)piperidine-1-carbonyl)phenyl) piperidin-1-yl)ethyl)-1-methyl-1H-pyrrolo[2,3-b]pyridin-4-yl)pyridin-2-yl) carbamate (0.11 g) was prepared similarly to Int. 2C18 from Ints. 3E76/2M7 (85 mg/0.12 g, HCl salt).

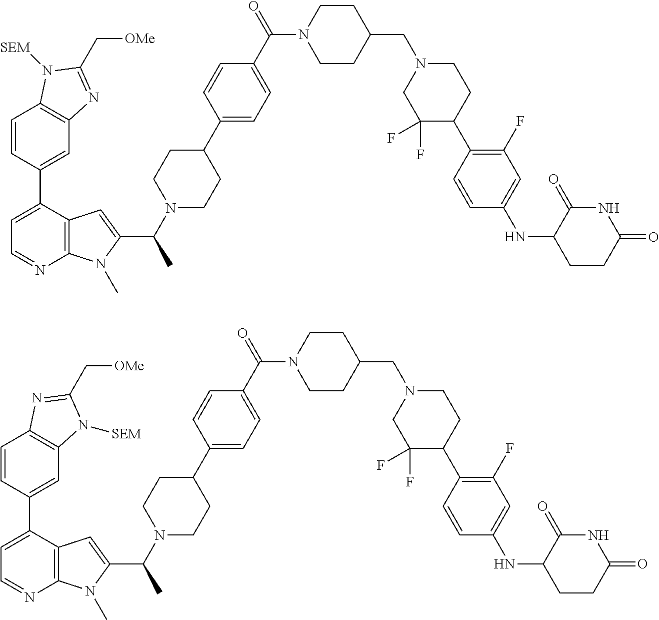 [SMILES: COCc1nc2cc(-c3ccnc4c3cc([C@H](C)N3CCC(c5ccc(C(=O)N6CCC(CN7CCC(c8ccc(NC9CCC(=O)NC9=O)cc8F)C(F)(F)C7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCC(c8ccc(NC9CCC(=O)NC9=O)cc8F)C(F)(F)C7)CC6)cc5)CC3)n4C)cc2n1COCC[Si](C)(C)C]

Int. 2M13 and 2M14. 3-((4-(3,3-difluoro-1-((1-(4-(1-((S)-1-(4-(2-(methoxymethyl)-1-((2-(trimethylsilyl) ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperidin-4-yl)-3-fluorophenyl)amino)piperidine-2,6-dione and 3-((4-(3,3-difluoro-1-((1-(4-(1-((S)-1-(4-(2-(methoxymethyl)-1-((2-(trimethylsilyl) ethoxy)-methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperidin-4-yl)-3-fluorophenyl)-amino)piperidine-2,6-dione (0.20 g) was prepared similarly to Int. 2C18 from Ints. 2F8/2F9/2M7 (0.20 g/0.24 g, HCl salt).

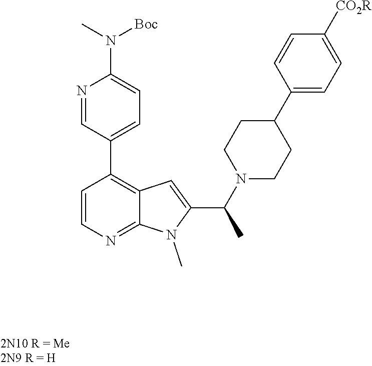

Int. 3E35 (50 mg), tert-butyl methyl (5-(4,4,5,5-tetramethyl-1,3,2-dioxaborolan-2-yl)pyridin-2-yl) carbamate (37 mg), NaHCO₃ (18 mg), and PdDPPFCl₂-DCM (9 mg) were degassed in dioxane/water (9:1, 2 mL) and stirred ON at 120° C. The OL (EtOAc/water) was dried, concentrated, and purified by FC (pentane/EtOAc 9:1 to 7:3) to give Int. 2N10 methyl 4-[1-[(1S)-1-[4-[6-[tert-butoxycarbonyl (methyl)-amino]-3-pyridyl]-1-methyl-pyrrolo[2,3-b]pyridin-2-yl]ethyl]-4-piperidyl]-benzoate (30 mg). LiOH—H₂O (76 mg) and Int. 2N10 (0.35 g) were stirred 1 h at 0° C. to RT in THF/MeOH/water (3:3:1, 5 mL) and neutralized with 1M aq. HCl to precipitate Int. 2N9 (S)-4-(1-(1-(4-(6-((tert-butoxycarbonyl)(methyl)amino)pyridin-3-yl)-1-methyl-1H-pyrrolo[2,3-b]pyridin-2-yl)ethyl)piperidin-4-yl)benzoic acid (0.21 g).

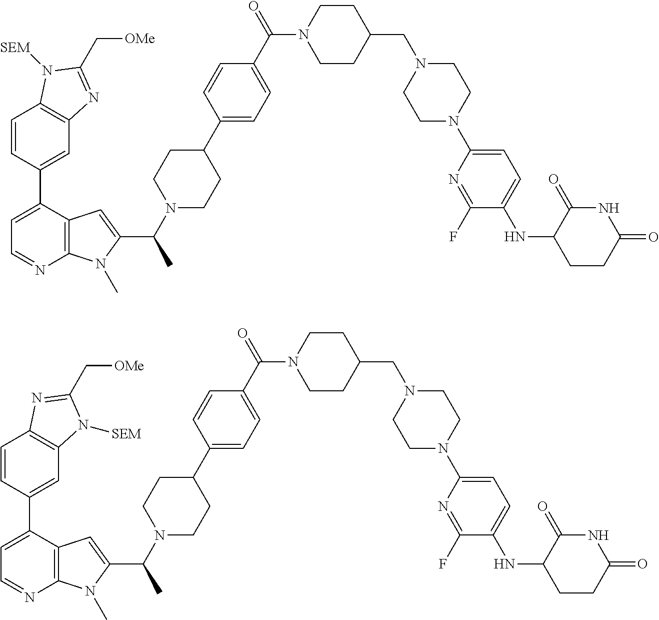 [SMILES: COCc1nc2cc(-c3ccnc4c3cc([C@H](C)N3CCC(c5ccc(C(=O)N6CCC(CN7CCN(c8ccc(NC9CCC(=O)NC9=O)c(F)n8)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ccc(NC9CCC(=O)NC9=O)c(F)n8)CC7)CC6)cc5)CC3)n4C)cc2n1COCC[Si](C)(C)C]

Int. 2N11 and 2N12. 3-((2-fluoro-6-(4-((1-(4-(1-((S)-1-(4-(2-(methoxymethyl)-1-((2-(trimethylsilyl)-ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperazin-1-yl)pyridin-3-yl)amino)piperidine-2,6-dione and 3-((2-fluoro-6-(4-((1-(4-(1-((S)-1-(4-(2-(methoxymethyl)-1-((2-(trimethylsilyl) ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)pyridin-3-yl)-amino)piperidine-2,6-dione (80 mg) was prepared similarly to Int. 2C18 from Ints. 2F8/2F9/2N14 (0.10 g/88 mg, HCl salt).

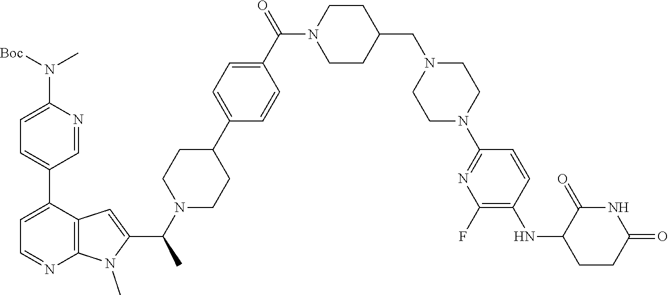 [SMILES: C[C@@H](c1cc2c(-c3ccc(N(C)C(=O)OC(C)(C)C)nc3)ccnc2n1C)N1CCC(c2ccc(C(=O)N3CCC(CN4CCN(c5ccc(NC6CCC(=O)NC6=O)c(F)n5)CC4)CC3)cc2)CC1]

Int. 2N13. tert-butyl (5-(2-((1S)-1-(4-(4-(4-((4-(5-((2,6-dioxopiperidin-3-yl)amino)-6-fluoropyridin-2-yl) piperazin-1-yl)methyl)piperidine-1-carbonyl)phenyl) piperidin-1-yl)ethyl)-1-methyl-1H-pyrrolo[2,3-b]pyridin-4-yl)pyridin-2-yl) (methyl) carbamate (0.20 g) was prepared similarly to Int. 2C18 from Int. 2N9/2N14 (0.20 g/0.22 g, HCl salt).

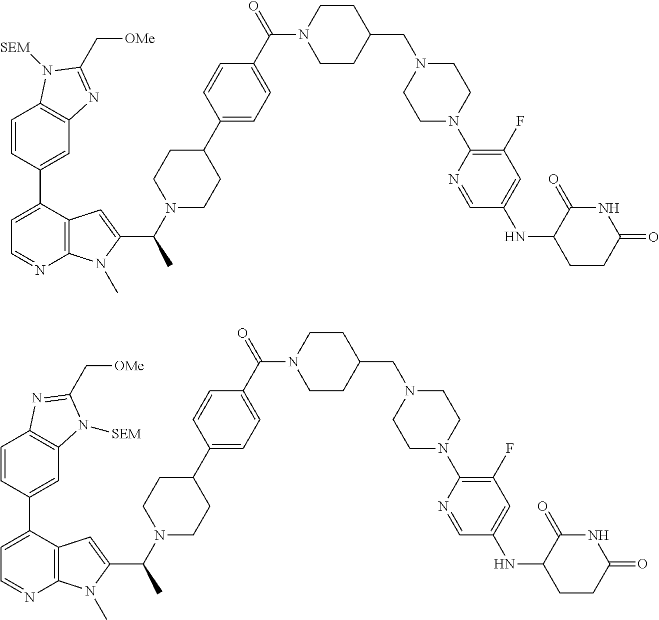 [SMILES: COCc1nc2cc(-c3ccnc4c3cc([C@H](C)N3CCC(c5ccc(C(=O)N6CCC(CN7CCN(c8ncc(NC9CCC(=O)NC9=O)cc8F)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ncc(NC9CCC(=O)NC9=O)cc8F)CC7)CC6)cc5)CC3)n4C)cc2n1COCC[Si](C)(C)C]

Int. 2N18 and 2N19. 3-((5-fluoro-6-(4-((1-(4-(1-((S)-1-(4-(2-(methoxymethyl)-1-((2-(trimethylsilyl)-ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperazin-1-yl)pyridin-3-yl)amino)piperidine-2,6-dione and 3-((5-fluoro-6-(4-((1-(4-(1-((S)-1-(4-(2-(methoxymethyl)-1-((2-(trimethylsilyl) ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)pyridin-3-yl)amino)-piperidine-2,6-dione (0.10 g) was prepared similarly to Int. 2C18 from Ints. 2F8/2F9/2N5 (80 mg/76 mg, HCl salt).

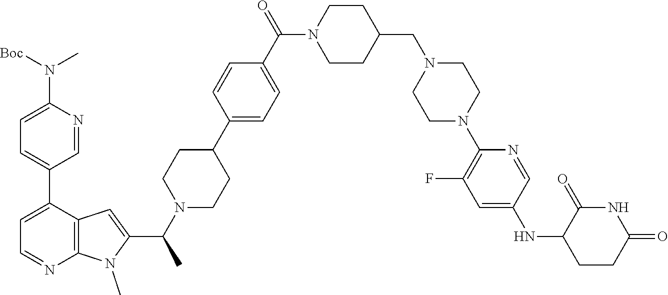 [SMILES: C[C@@H](c1cc2c(-c3ccc(N(C)C(=O)OC(C)(C)C)nc3)ccnc2n1C)N1CCC(c2ccc(C(=O)N3CCC(CN4CCN(c5ncc(NC6CCC(=O)NC6=O)cc5F)CC4)CC3)cc2)CC1]

Int. 2N20. tert-butyl (5-(2-((1S)-1-(4-(4-(4-((4-(5-((2,6-dioxopiperidin-3-yl)amino)-3-fluoropyridin-2-yl) piperazin-1-yl)methyl)piperidine-1-carbonyl)phenyl) piperidin-1-yl)ethyl)-1-methyl-1H-pyrrolo[2,3-b]pyridin-4-yl)pyridin-2-yl) (methyl) carbamate (0.20 g) was prepared similarly to Int. 2C18 from Ints. 2N9/2N5 (0.15 g/0.16 g, HCl salt).

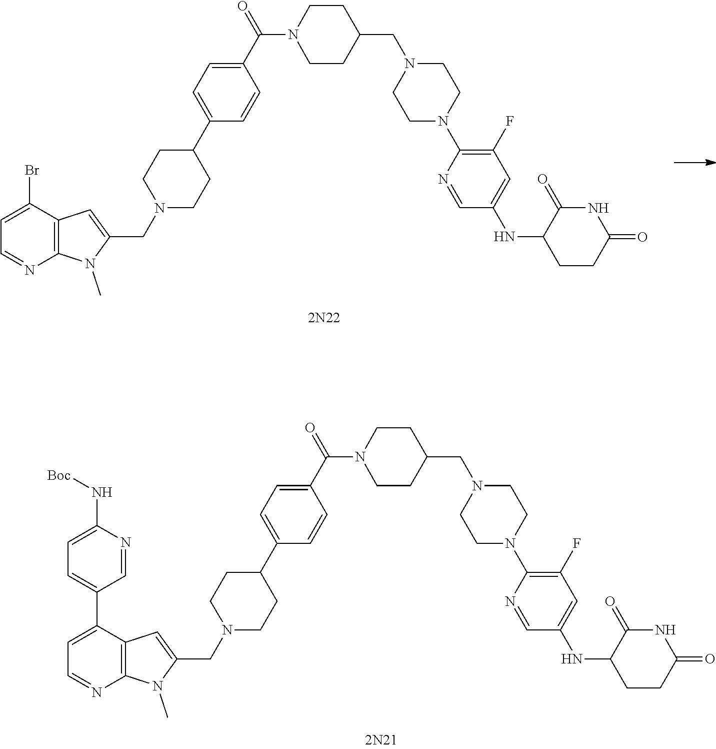 [SMILES: Cn1c(CN2CCC(c3ccc(C(=O)N4CCC(CN5CCN(c6ncc(NC7CCC(=O)NC7=O)cc6F)CC5)CC4)cc3)CC2)cc2c(Br)ccnc21 ; Cn1c(CN2CCC(c3ccc(C(=O)N4CCC(CN5CCN(c6ncc(NC7CCC(=O)NC7=O)cc6F)CC5)CC4)cc3)CC2)cc2c(-c3ccc(NC(=O)OC(C)(C)C)nc3)ccnc21]

Int. 2N22 3-((6-(4-((1-(4-(1-((4-bromo-1-methyl-1H-pyrrolo[2,3-b]pyridin-2-yl)methyl)-piperidin-4-yl)benzoyl)-piperidin-4-yl)methyl)piperazin-1-yl)-5-fluoropyridin-3-yl)amino)piperidine-2,6-dione (57 mg) was prepared similarly to Int. 2C18 from Ints. 1AC4/2N5 (70 mg/86 mg, HCl salt). tert-Butyl (5-(4,4,5,5-tetramethyl-1,3,2-dioxaborolan-2-yl)pyridin-2-yl)-carbamate (29 mg), Int. 2N22 (53 mg), and PdDPPFCl₂-DCM (5 mg) were degassed in DMF/10% aq. Na₂CO₃ (5:1, 0.84 mL) and stirred 0.5 h at 100° C. The OL (EtOAc/water) was washed with brine, dried, concentrated, and purified by FC (DCM/MeOH 1:0 to 9:1) to give Int. 2N21 tert-butyl (5-(2-((4-(4-(4-((4-(5-((2,6-dioxopiperidin-3-yl)amino)-3-fluoropyridin-2-yl) piperazin-1-yl)-methyl)piperidine-1-carbonyl)phenyl) piperidin-1-yl)methyl)-1-methyl-1H-pyrrolo[2,3-b]pyridin-4-yl)pyridin-2-yl) carbamate (20 mg).

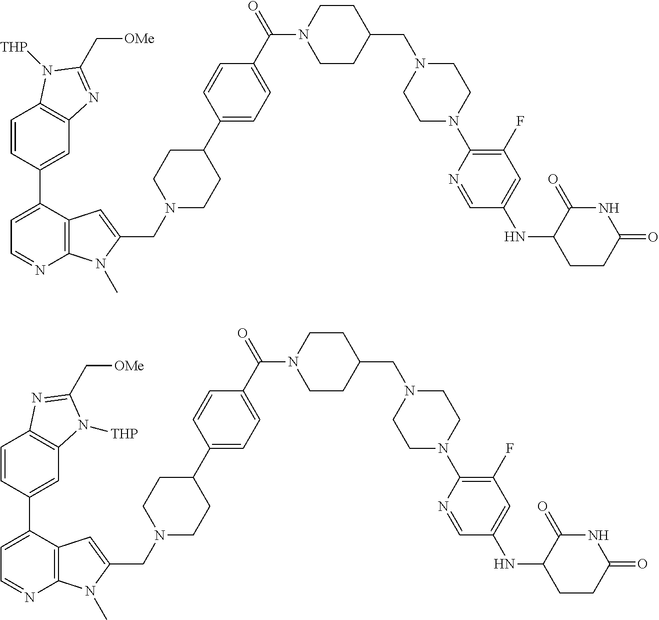

Int. 2N23 and 2N24. 3-((5-fluoro-6-(4-((1-(4-(1-((4-(2-(methoxymethyl)-1-(tetrahydro-2H-pyran-2-yl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperazin-1-yl)pyridin-3-yl)amino)piperidine-2,6-dione and 3-((5-fluoro-6-(4-((1-(4-(1-((4-(2-(methoxymethyl)-1-(tetrahydro-2H-pyran-2-yl)-1H-benzo[d]imidazol-6-yl)-1-methyl-1H-pyrrolo[2,3-b]pyridin-2-yl)methyl)piperidin-4-yl)benzoyl)piperidin-4-yl)methyl)piperazin-1-yl)pyridin-3-yl)amino)piperidine-2,6-dione (15 mg) was prepared similarly to Int. 2C18 from Ints. 2C55/2N5 (29 mg/21 mg, HCl salt).

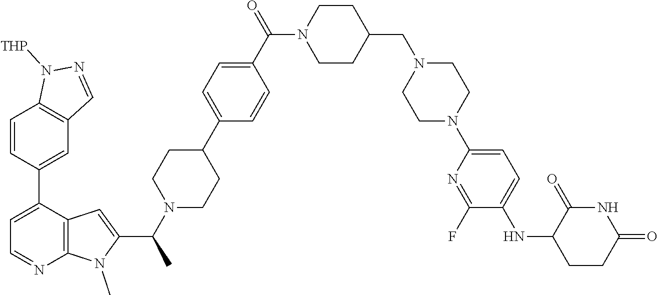

Int. 2N25. 3-((2-fluoro-6-(4-((1-(4-(1-((1S)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoyl)piperidin-4-yl)methyl)piperazin-1-yl)pyridin-3-yl)amino)piperidine-2,6-dione and 3-((2-fluoro-6-(4-((1-(4-(1-((1S)-1-(4-(2-(methoxymethyl)-1-(tetrahydro-2H-pyran-2-yl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)pyridin-3-yl)amino)piperidine-2,6-dione (106 mg) was prepared similarly to Int. 2C18 from Int. 2C54 (92 mg) and 2N14 (90 mg, HCl salt).

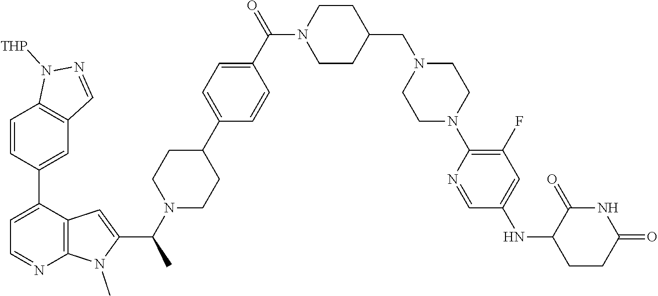

Int. 2N26. 3-((5-fluoro-6-(4-((1-(4-(1-((1S)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoyl)piperidin-4-yl)methyl)piperazin-1-yl)pyridin-3-yl)amino)piperidine-2,6-dione (63 mg) was prepared similarly to Int. 2C18 from Ints. 2C54/2N5 (53 mg/52 mg, HCl salt).

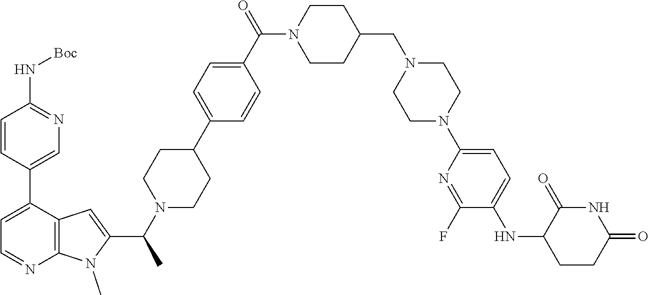 [SMILES: C[C@@H](c1cc2c(-c3ccc(NC(=O)OC(C)(C)C)nc3)ccnc2n1C)N1CCC(c2ccc(C(=O)N3CCC(CN4CCN(c5ccc(NC6CCC(=O)NC6=O)c(F)n5)CC4)CC3)cc2)CC1]

Int. 2N27. tert-butyl (5-(2-((1S)-1-(4-(4-(4-((4-(5-((2,6-dioxopiperidin-3-yl)amino)-6-fluoropyridin-2-yl) piperazin-1-yl)methyl)piperidine-1-carbonyl)phenyl) piperidin-1-yl)ethyl)-1-methyl-1H-pyrrolo[2,3-b]pyridin-4-yl)pyridin-2-yl) carbamate (104 mg) was prepared similarly to Int. 2C18 from Ints. 3E76/2N14 (90 mg/89 mg, HCl salt).

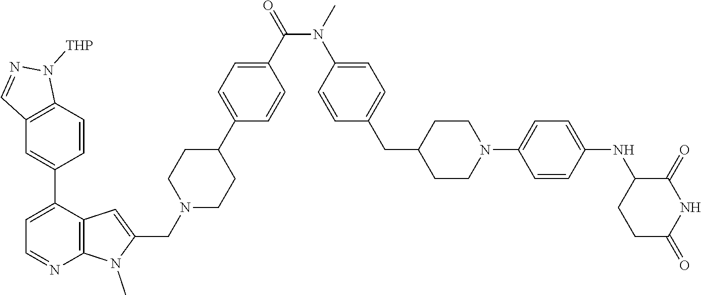

Int. 2O1 (50 mg), 1-(tetrahydro-2H-pyran-2-yl)-5-(4,4,5,5-tetramethyl-1,3,2-dioxaborolan-2-yl)-1H-indazole (19 mg), and PdDPPFCl₂-DCM (4 mg) were degassed in DMF/10% aq. Na₂CO₃ (15:1, 1.1 mL) and stirred 1.5 h at 100° C. The filtrate after filtration was purified by FC (DCM/MeOH 1:0 to 9:1) to give Int. 2O6 N-(4-((1-(4-((2,6-dioxopiperidin-3-yl)amino)phenyl) piperidin-4-yl)methyl) phenyl)-N-methyl-4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzamide (46 mg).

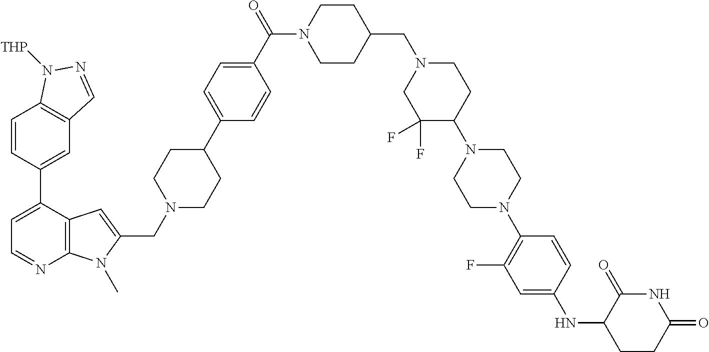

Int. 2P1. 3-((4-(4-(3,3-difluoro-1-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)piperidin-4-yl) piperazin-1-yl)-3-fluorophenyl)amino)piperidine-2,6-dione (25 mg) was prepared similarly to Int. 2C18 from HCl salts of Ints. 3A2/2P2 (25 mg) and 2P2 (22 mg).

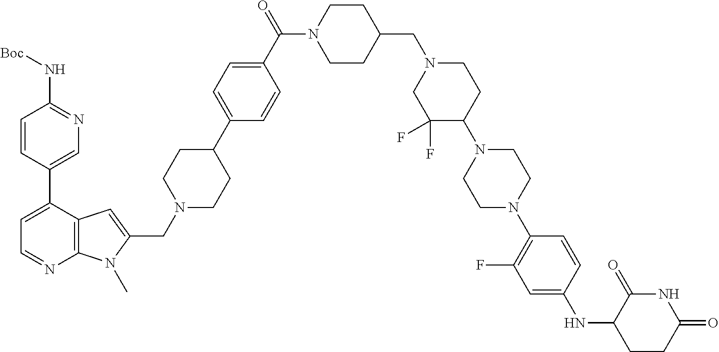 [SMILES: Cn1c(CN2CCC(c3ccc(C(=O)N4CCC(CN5CCC(N6CCN(c7ccc(NC8CCC(=O)NC8=O)cc7F)CC6)C(F)(F)C5)CC4)cc3)CC2)cc2c(-c3ccc(NC(=O)OC(C)(C)C)nc3)ccnc21]

Int. 2P9. tert-butyl (5-(2-((4-(4-(4-((4-(4-(4-((2,6-dioxopiperidin-3-yl)amino)-2-fluorophenyl) piperazin-1-yl)-3,3-difluoropiperidin-1-yl)methyl)piperidine-1-carbonyl)phenyl) piperidin-1-yl)methyl)-1-methyl-1H-pyrrolo[2,3-b]pyridin-4-yl)pyridin-2-yl) carbamate (27 mg) was prepared similarly to Int. 2C18 from Int. 2E6 (20 mg, HCl salt) and 2P2 (22 mg, HCl salt).

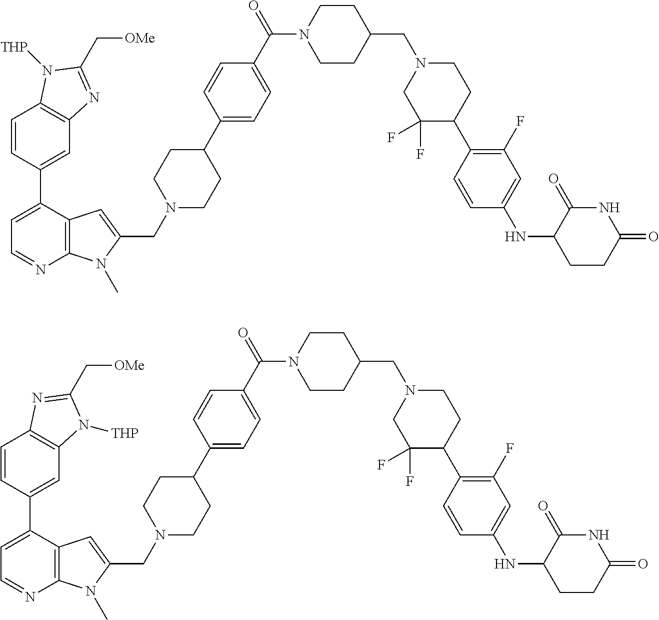

Int. 2P10 and 2P11. 3-((4-(4-(3,3-difluoro-1-((1-(4-(1-((4-(2-(methoxymethyl)-1-(tetrahydro-2H-pyran-2-yl)-1H-benzo[d]imidazol-5-yl)-1-methyl-1H-pyrrolo[2,3-b]pyridin-2-yl)methyl)piperidin-4-yl)benzoyl)-piperidin-4-yl)-methyl)piperidin-4-yl) piperazin-1-yl)-3-fluorophenyl)amino)piperidine-2,6-dione and 3-((4-(3,3-difluoro-1-((1-(4-(1-((4-(2-(methoxymethyl)-1-(tetrahydro-2H-pyran-2-yl)-1H-benzo[d] imidazol-6-yl)-1-methyl-1H-pyrrolo[2,3-b]pyridin-2-yl)methyl)piperidin-4-yl)benzoyl)piperidin-4-yl)methyl)piperidin-4-yl)-3-fluorophenyl)amino)-piperidine-2,6-dione (20 mg) was prepared similarly to Int. 2C18 from Ints. 2C55/2P2 (18 mg/19 mg, HCl salt).

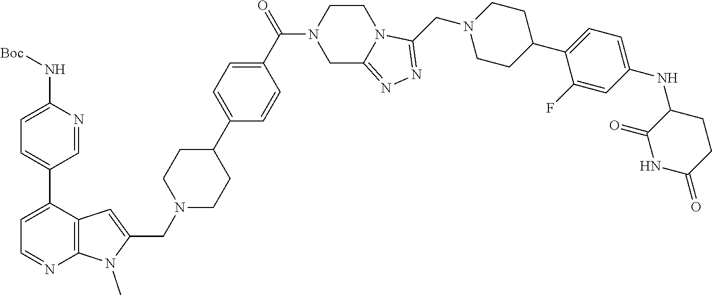 [SMILES: Cn1c(CN2CCC(c3ccc(C(=O)N4CCn5c(CN6CCC(c7ccc(NC8CCC(=O)NC8=O)cc7F)CC6)nnc5C4)cc3)CC2)cc2c(-c3ccc(NC(=O)OC(C)(C)C)nc3)ccnc21]

Int. 2Q1. tert-butyl (5-(2-((4-(4-(3-((4-(4-((2,6-dioxopiperidin-3-yl)amino)-2-fluorophenyl)-piperidin-1-yl)methyl)-5,6,7,8-tetrahydro-[1,2,4]triazolo [4,3-a]pyrazine-7-carbonyl)phenyl) piperidin-1-yl)methyl)-1-methyl-1H-pyrrolo[2,3-b]pyridin-4-yl)pyridin-2-yl) carbamate (80 mg) was prepared similarly to Int. 2C18 from Ints. 2E6/2Q2 (44 mg/55 mg, HCl salt).

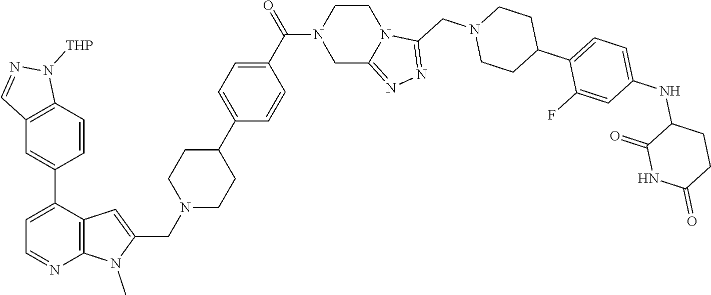

Int. 2Q9. 3-((3-fluoro-4-(1-((7-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)-5,6,7,8-tetrahydro-[1,2,4]triazolo [4,3-a]pyrazin-3-yl)methyl)-piperidin-4-yl)phenyl)amino)-piperidine-2,6-dione (0.12 g) was prepared similarly to Int. 2C18 from Ints. 3A2 (44 mg) and 2Q2 (55 mg, HCl salt).

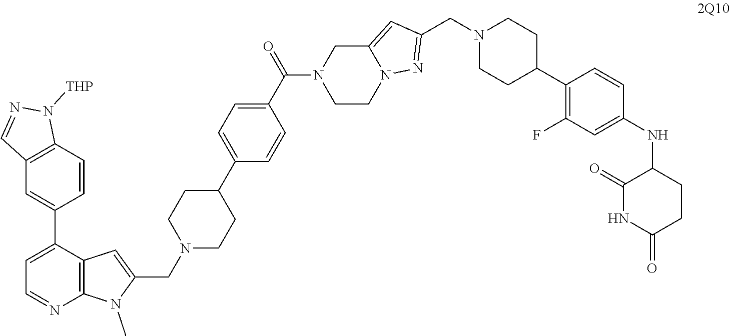

Int. 2Q10 3-((3-fluoro-4-(1-((5-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)-4,5,6,7-tetrahydropyrazolo [1,5-a]pyrazin-2-yl)methyl)piperidin-4-yl)phenyl)amino)piperidine-2,6-dione (60 mg) was prepared similarly to Int. 2C18 from Ints. 3A2/2Q15(46 mg/40 mg, HCl salt).

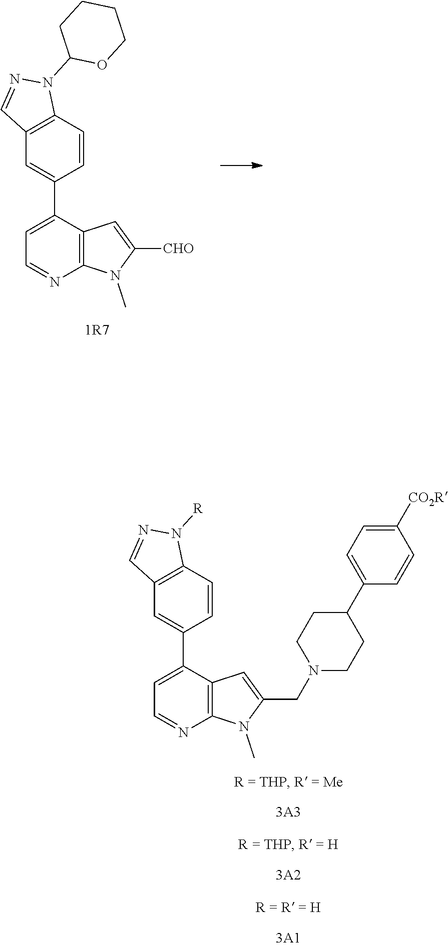

Methyl 4-(4-piperidyl)benzoate (0.13 g) and Int. 1R7 (0.11 g) were stirred 0.75 h in DCE (4 mL). STAB (0.19 g) was added and stirring continued 4 h. The OL (DCM/sat. aq. NaHCO₃) was concentrated and HPLC-purified to give Int. 3A3 methyl 4-(1-((1-methyl-4-(1-(tetrahydro-2H-pyran-2-yl)-1H-indazol-5-yl)-1H-pyrrolo[2,3-b]-pyridin-2-yl)-methyl)-piperidin-4-yl)benzoate (0.12 g). Int. 3A3 (2.0 g) was stirred ON in THF/MeOH/2M aq. NaOH (2:1:1; 22 mL). 2M aq. NaOH (2 mL) was added and stirring continued 0.5 h. The mixture was neutralized with aq. HCl and concentrated to give Int. 3A2 4-(1-((1-methyl-4-(1-(tetrahydro-2H-pyran-2-yl)-1H-indazol-5-yl)-1H-pyrrolo[2,3-b]pyridin-2-yl)methyl)-piperidin-4-yl)benzoic acid (1.95 g, HCl salt). 1.0 g of this material was stirred 0.75 h in DCM/TFA (2:1; 3 mL). The residue after concentration was triturated in MeOH to give Int. 3A1 4-[1-[[4-(1H-indazol-5-yl)-1-methyl-pyrrolo[2,3-b]pyridin-2-yl]methyl]-4-piperidyl]benzoic acid (0.62 g).

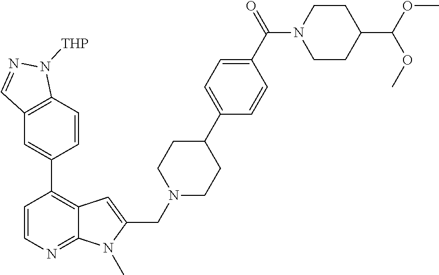

Int. 3A2 (0.50 g, HCl salt) and HATU (0.49 g) were stirred 0.3 h in DMF/DIPEA (20:1, 10.5 mL). 4-(Dimethoxy-methyl)piperidine (0.16 g) was added and stirring continued ON. The OL (water/EtOAc) was washed with brine, dried, concentrated, and purified by FC (DCM/MeOH 1:0 to 95:5) to give Int. 3D4 (4-(dimethoxy-methyl)-piperidin-1-yl) (4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)phenyl) methanone (0.20 g).

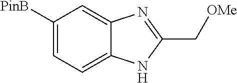

5-Bromo-2-(methoxymethyl)-1H-benzo[d]imidazole (2.0 g), B₂Pin₂ (5.3 g), KOAc (2.4 g), and PdDPPFCl₂-DCM (0.34 g) were degassed in dioxane (20 mL) and stirred ON at 100° C., concentrated, and purified by FC (hexane/EtOAc 1:4) to give Int. 3E8 2-(methoxymethyl)-5-(4,4,5,5-tetramethyl-1,3,2-dioxaborolan-2-yl)-1H-benzo[d]-imidazole (2.1 g).

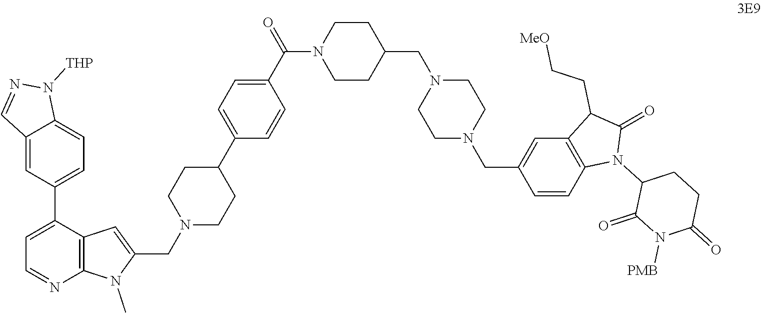

Int. 3E12 (30 mg), Int. 3A2 (34 mg), and HATU (24 mg) were stirred 2 h in DMF/DIPEA (23.8:1, 2.1 mL), filtered, and HPLC-purified to give Int. 3E9 1-(4-methoxybenzyl)-3-(3-(2-methoxyethyl)-5-((4-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)piperazin-1-yl)methyl)-2-oxo-2,3-dihydro-1H-benzo[d]imidazol-1-yl) piperidine-2,6-dione (51 mg).

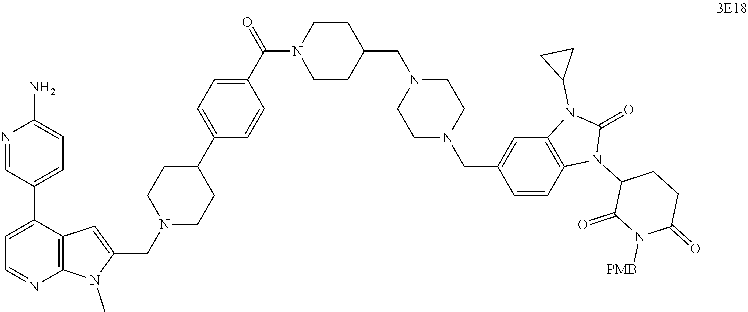

Int. 3E18 3-(5-((4-((1-(4-(1-((4-(6-aminopyridin-3-yl)-1-methyl-1H-pyrrolo[2,3-b]pyridin-2-yl)methyl)piperidin-4-yl)benzoyl)piperidin-4-yl)methyl)piperazin-1-yl)methyl)-3-cyclo-propyl-2-oxo-2,3-dihydro-1H-benzo[d]imidazol-1-yl)-1-(4-methoxybenzyl) piperidine-2,6-dione (33 mg) was prepared similarly to Int. 3E9 from Ints. 3E23/3E19 (29 mg/36 mg).

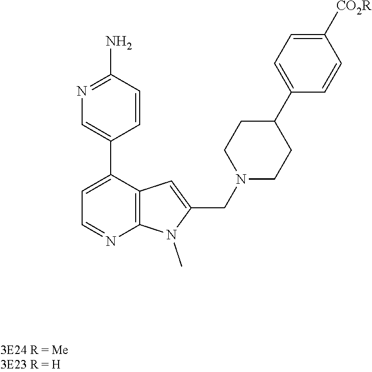

Int. 1AC5 (0.50 g), 5-(4,4,5,5-tetramethyl-1,3,2-dioxaborolan-2-yl)pyridin-2-amine (0.37 g), Na₂CO₃ (2.4 g), and PdDPPFCl₂-DCM (92 mg) were degassed in DMF/water (5.7:1, 6.7 mL) and stirred 1 h at 100° C. Filtration, concentration, and purification by FC (heptane/EtOAc 1:2 to 0:1) gave to give Int. 3E24 methyl 4-(1-((4-(6-aminopyridin-3-yl)-1-methyl-1H-pyrrolo[2,3-b]pyridin-2-yl)methyl)piperidin-4-yl)benzoate (0.39 g). Int. 3E24 (1.0 g) was stirred 5 h in THF/MeOH (2:1; 39 mL) and 2M aq. NaOH (5.7 mL). The mixture was acidified with 5M aq. HCl (pH 1-2), neutralized with 1M aq. LiOH, concentrated, and HPLC-purified to give Int. 3E23 4-(1-((4-(6-aminopyridin-3-yl)-1-methyl-1H-pyrrolo[2,3-b]pyridin-2-yl)methyl)piperidin-4-yl)benzoic acid (0.42 g).

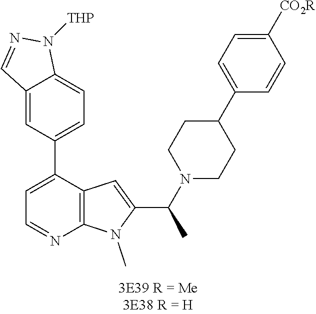

Int. 3E35 (60 mg), 1-(tetrahydro-2H-pyran-2-yl)-5-(4,4,5,5-tetramethyl-1,3,2-dioxaborolan-2-yl)-1H-indazole (60 mg), Na₂CO₃ (0.28 mL of a 10% aq. solution), and PdDPPFCl₂-DCM (11 mg) were degassed in DMF (0.8 mL) and stirred 1 h at 100° C. The OL (aq. NaHCO₃/EtOAc) was washed with brine, dried, concentrated, and purified by FC (heptane/EtOAc 1:2) to give Int. 3E39 methyl 4-(1-((1S)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoate or methyl 4-(1-((1R)-1-(1-methyl-4-(1-(tetra-hydro-2H-pyran-2-yl)-1H-indazol-5-yl)-1H-pyrrolo[2,3-b]pyridin-2-yl)-ethyl)piperidin-4-yl)benzoate (63 mg). Int. 3E39 (59 mg) was stirred in THF/MeOH/1M aq. NaOH (1.3:2.8:1, 2 mL) at 0° C. to RT ON. The mixture was partially concentrated. The OL (brine/2M aq. HCl/DCM: MeOH (9:1)) was dried, concentrated, and purified by FC (DCM/MeOH 95:5) to give 4-(1-((1S)-1-(1-methyl-4-(1-(tetra-hydro-2H-pyran-2-yl)-1H-indazol-5-yl)-1H-pyrrolo-[2,3-b]pyridin-2-yl)ethyl)piperidin-4-yl)benzoic acid or 4-(1-((1R)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoic acid (47 mg). 43 mg of this material was stirred 1.5 h in DCM/TFA (4:1; 1 mL) and concentrated to give Int. 3E38 (S)-4-(1-(1-(4-(1H-indazol-5-yl)-1-methyl-1H-pyrrolo[2,3-b]pyridin-2-yl)ethyl)piperidin-4-yl)benzoic acid or (R)-4-(1-(1-(4-(1H-indazol-5-yl)-1-methyl-1H-pyrrolo[2,3-b]pyridin-2-yl)ethyl)piperidin-4-yl)benzoic acid (64 mg).

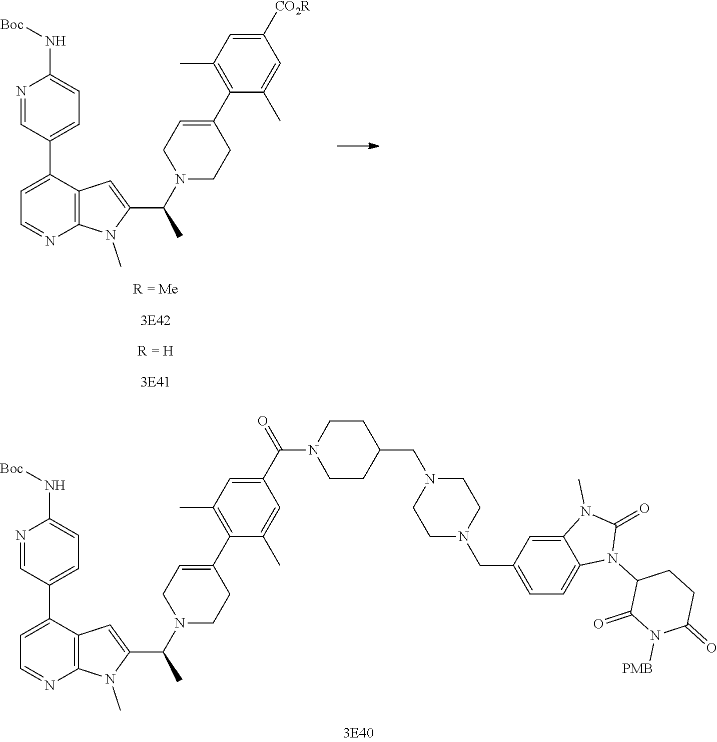

Int. 3E43 (0.32 g), tert-butyl (5-(4,4,5,5-tetramethyl-1,3,2-dioxaborolan-2-yl)pyridin-2-yl) carbamate (0.24 g), NaHCO₃ (86 mg), and PdDPPFCl₂-DCM (28 mg) were degassed in dioxane/water (9:1; 8 mL) and stirred ON at 90° C. The OL (water/EtOAc) was concentrated and purified by FC (hexane/EtOAc 7:3) to give Int. 3E42 methyl 4-[1-[4-[6-(tert-butoxycarbonylamino)-3-pyridyl]-1-methyl-pyrrolo[2,3-b]pyridin-2-yl]methyl]-3,6-dihydro-2H-pyridin-4-yl]-3,5-dimethyl-benzoate (0.14 g). Int. 3E42 (0.82 g) and LiOH—H₂O (0.30 g) were stirred in THF/MeOH/water (6:2:1; 7 mL) ON. The mixture was neutralized with 0.5M aq. HCl to precipitate Int. 3E41 4-(1-((4-(6-((tert-butoxy-carbonyl)amino)-pyridin-3-yl)-1-methyl-1H-pyrrolo[2,3-b]pyridin-2-yl)methyl)-1,2,3,6-tetrahydropyridin-4-yl)-3,5-dimethylbenzoic acid (0.45 g). Int. 3E2 (30 mg) was stirred 0.5 h in DCM/TFA (4:1; 1 mL) and concentrated. The residue, Int. 3E41 (22 mg), and HATU (18 mg) were stirred 0.75 h in DCM/DIPEA (8.6:1, 0.67 mL). The OL (sat. aq. NaHCO₃/DCM) was washed with brine, dried, concentrated, and purified by FC (DCM/MeOH 96:4) to give Int. 3E40 tert-butyl (5-(2-((4-(4-(4-((4-((1-(1-(4-methoxybenzyl)-2,6-dioxopiperidin-3-yl)-3-methyl-2-oxo-2,3-dihydro-1H-benzo[d] imidazol-5-yl)methyl)piperazin-1-yl)methyl)piperidine-1-carbonyl)-2,6-dimethylphenyl)-3,6-dihydropyridin-1(2H)-yl)methyl)-1-methyl-1H-pyrrolo[2,3-b]pyridin-4-yl)pyridin-2-yl) carbamate (38 mg).

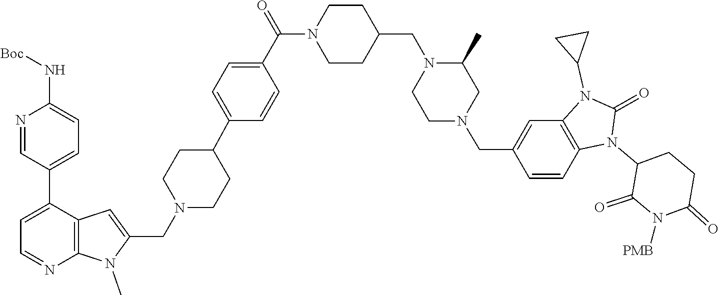

Int. 3E32 (0.22 g), PdDPPFCl₂-DCM (18 mg), Na₂CO₃ (0.46 mL of a 10% aq. solution), and tert-butyl (5-(4,4,5,5-tetramethyl-1,3,2-dioxaborolan-2-yl)pyridin-2-yl) carbamate (0.11 g) were degassed in DMF (2.2 mL) and stirred 2 h at 100° C. Filtration, concentration, and purification by FC (DCM/MeOH 95:5 t 9:1) gave Int. 3E46 tert-butyl (5-(2-((4-(4-(4-(((2S)-4-((3-cyclo-propyl-1-(1-(4-methoxybenzyl)-2,6-dioxopiperidin-3-yl)-2-oxo-2,3-dihydro-1H-benzo[d]imidazol-5-yl)methyl)-2-methylpiperazin-1-yl)methyl)piperidine-1-carbonyl)phenyl) piperidin-1-yl)methyl)-1-methyl-1H-pyrrolo[2,3-b]pyridin-4-yl)pyridin-2-yl) carbamate (87 mg).

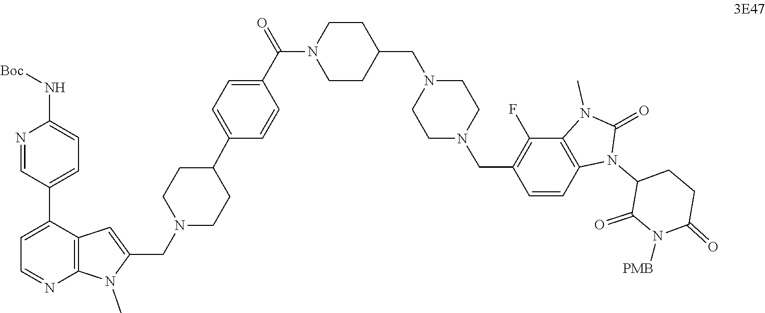

Int. 3E48 (0.10 g, TFA salt), Int. 3E56 (66 mg), and HATU (52 mg) were stirred ON in DMF/DIPEA (10:1, 2.2 mL). The OL (water/EtOAc) was washed with brine, dried, concentrated, and purified by FC (DCM/MeOH 1:0 to 9:1) to give Int. 3E47 tert-butyl (5-(2-((4-(4-(4-((4-((4-fluoro-1-(1-(4-methoxybenzyl)-2,6-dioxopiperidin-3-yl)-3-methyl-2-oxo-2,3-dihydro-1H-benzo[d]imidazol-5-yl)methyl)piperazin-1-yl)methyl)piperidine-1-carbonyl)phenyl) piperidin-1-yl)methyl)-1-methyl-1H-pyrrolo[2,3-b]pyridin-4-yl)pyridin-2-yl) carbamate (95 mg).

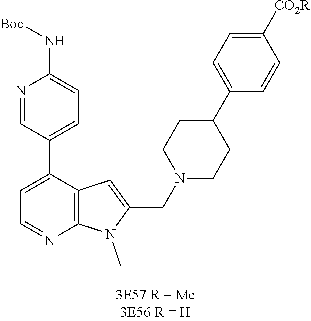

Int. 1AC5 (1.0 g), tert-butyl (5-(4,4,5,5-tetramethyl-1,3,2-dioxaborolan-2-yl)pyridin-2-yl) carbamate (0.8 g), NaHCO₃ (0.38 g), and PdDPPFCl₂-DCM (0.18 g) were degassed in dioxane/water (5:1; 12 mL) and stirred ON at 100° C., filtered, and concentrated. The residue was combined with three other batches prepared similarly and purified by FC (pentane/EtOAc 1:0 to 3:2) to give Int. 3E57 methyl 4-(1-((4-(6-((tert-butoxy-carbonyl)amino)-pyridin-3-yl)-1-methyl-1H-pyrrolo[2,3-b]pyridin-2-yl)methyl)piperidin-4-yl)benzoate (3.5 g). 1.8 g of this material and LiOH—H₂O (0.65 g) were stirred ON in MeOH/THF/water (2:2:1; 25 mL) at 0° C. to RT, concentrated, and triturated in dilute aq. HCl to give Int. 3E56 4-(1-((4-(6-((tert-butoxycarbonyl)amino)pyridin-3-yl)-1-methyl-1H-pyrrolo[2,3-b]pyridin-2-yl)methyl)piperidin-4-yl)benzoic acid (1.2 g, HCl salt).

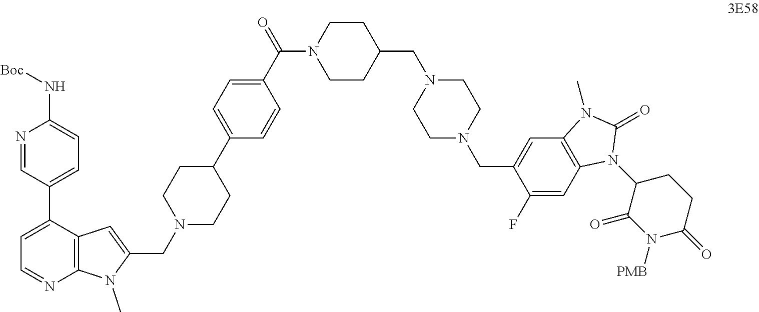

Int. 3E60 (41 mg) was stirred 1 h in DCM/TFA (1:1; 2 mL) and concentrated. The residue, Int. 3E56 (39 mg) and HATU (28 mg) were stirred ON in DMF/DIPEA (20:1, 2.1 mL), filtered, and HPLC-purified to give Int. 3E58 tert-butyl (5-(2-((4-(4-(4-((4-((6-fluoro-1-(1-(4-methoxybenzyl)-2,6-dioxopiperidin-3-yl)-3-methyl-2-oxo-2,3-dihydro-1H-benzo[d]-imidazol-5-yl)methyl)piperazin-1-yl)methyl)piperidine-1-carbonyl)phenyl) piperidin-1-yl)methyl)-1-methyl-1H-pyrrolo[2,3-b]pyridin-4-yl)pyridin-2-yl) carbamate (51 mg).

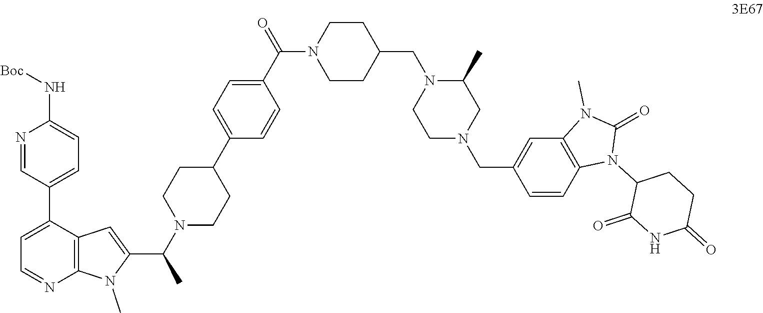 [SMILES: C[C@H]1CN(Cc2ccc3c(c2)n(C)c(=O)n3C2CCC(=O)NC2=O)CCN1CC1CCN(C(=O)c2ccc(C3CCN([C@@H](C)c4cc5c(-c6ccc(NC(=O)OC(C)(C)C)nc6)ccnc5n4C)CC3)cc2)CC1]

Ints. 3E76/3E68 (0.50 g/0.61 g) and HATU (0.44 g) were stirred ON in DMF/DIPEA (6.4:1, 5.8 mL) and diluted with water to precipitate Int. 3E67 tert-Butyl (5-(2-((1S)-1-(4-(4-(4-(((2S)-4-((1-(2,6-dioxopiperidin-3-yl)-3-methyl-2-oxo-2,3-dihydro-1H-benzo[d]imidazol-5-yl)methyl)-2-methylpiperazin-1-yl)methyl)piperidine-1-carbonyl)phenyl) piperidin-1-yl)ethyl)-1-methyl-1H-pyrrolo[2,3-b]pyridin-4-yl)pyridin-2-yl) carbamate (0.52 g).

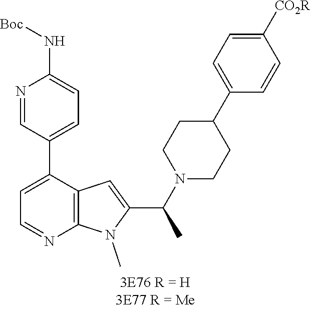

PdDPPFCl₂-DCM (0.8 g), tert-butyl (5-(4,4,5,5-tetramethyl-1,3,2-dioxaborolan-2-yl)pyridin-2-yl) carbamate (4.1 g), NaHCO₃ (1.7 g), and Int. 3E35(4.5 g) were degassed in dioxane/water (5:2; 12 mL) and stirred ON at 80° C. The OL (water/EtOAc) was washed with brine, dried, concentrated, and purified by FC (pentane/EtOAc 7:3 to 3:2) to give Int. 3E77 methyl(S)-4-(1-(1-(4-(6-((tert-butoxycarbonyl)amino)pyridin-3-yl)-1-methyl-1H-pyrrolo-[2,3-b]-pyridin-2-yl)ethyl)piperidin-4-yl)benzoate (3.5 g). This material and LiOH—H₂O (1.3 g) were stirred ON in THF/MeOH/water (2:2:1; 37 mL), concentrated, and triturated in dilute aq. citric acid (pH 5) to give Int. 3E76 (S)-4-(1-(1-(4-(6-((tert-butoxycarbonyl)amino)pyridin-3-yl)-1-methyl-1H-pyrrolo[2,3-b]pyridin-2-yl)ethyl)piperidin-4-yl)benzoic acid (3.1 g).

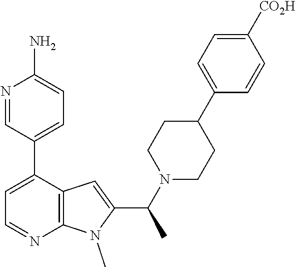 [SMILES: C[C@@H](c1cc2c(-c3ccc(N)nc3)ccnc2n1C)N1CCC(c2ccc(C(=O)O)cc2)CC1]

Int. 3E76 (9.5 g) was stirred ON in dioxane/4M HCl in dioxane (5:4; 90 mL) at 0° C. to RT, concentrated to give Int. 3E83 (S)-4-(1-(1-(4-(6-aminopyridin-3-yl)-1-methyl-1H-pyrrolo[2,3-b]pyridin-2-yl)ethyl)piperidin-4-yl)benzoic acid (8.4 g, HCl salt). Alternatively, Example 1af11 (50 mg) and NaOH (41 mg) were stirred ON at 100° C. in dioxane/water (8:1, 4.5 mL). The mixture was concentrated and triturated in DCM to give a solid. This material was taken up in aq. citric acid to precipitate Int. 3E83 (20 mg).

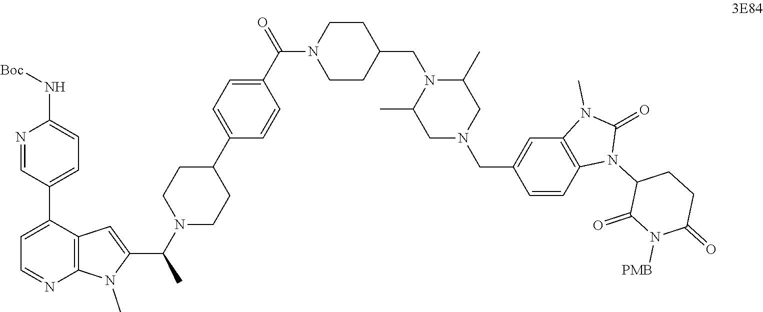

Ints. 3E85/3E76 (0.15 g, HCl salt/0.10 g) and HATU (68 mg) were stirred ON in DMF/DIPEA (24:1, 3.2 mL) and diluted with water to precipitate Int. 3E84 tert-butyl (5-(2-((1S)-1-(4-(4-(4-((4-((1-(1-(4-methoxybenzyl)-2,6-dioxopiperidin-3-yl)-3-methyl-2-oxo-2,3-dihydro-1H-benzo[d]imidazol-5-yl)methyl)-2,6-cis-dimethylpiperazin-1-yl)methyl)piperidine-1-carbonyl)phenyl) piperidin-1-yl)ethyl)-1-methyl-1H-pyrrolo[2,3-b]pyridin-4-yl)pyridin-2-yl) carbamate (0.14 g).

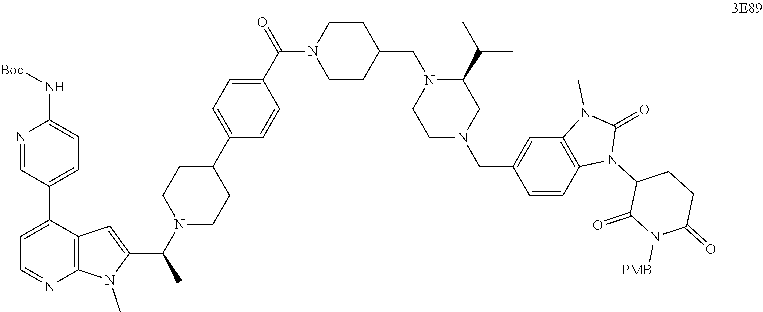

Ints. 3E90/3E76 (0.16 g/0.13 g) and HATU (0.14 g) were stirred ON in DMF/DIPEA (8:1, 2.25 mL, and diluted with water to precipitate Int. 3E89 tert-butyl (5-(2-((1S)-1-(4-(4-(4-(((2S)-2-iso-propyl-4-((1-(1-(4-methoxybenzyl)-2,6-dioxo-piperidin-3-yl)-3-methyl-2-oxo-2,3-dihydro-1H-benzo[d] imidazol-5-yl)methyl)piperazin-1-yl)methyl)-piperidine-1-carbonyl)phenyl) piperidin-1-yl)ethyl)-1-methyl-1H-pyrrolo[2,3-b]pyridin-4-yl)pyridin-2-yl) carbamate (0.19 g).

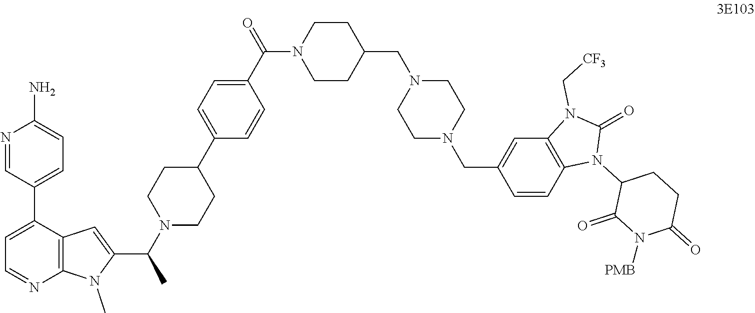

Ints. 3E83/3E104 (84 mg/0.16 g) and HATU (0.11 g) were stirred ON in DMF/DIPEA (26.3:1, 5.2 mL) and diluted with water to precipitate Int. 3E103 3-(5-((4-((1-(4-(1-((S)-1-(4-(6-aminopyridin-3-yl)-1-methyl-1H-pyrrolo[2,3-b]pyridin-2-yl)ethyl)piperidin-4-yl)benzoyl)piperidin-4-yl)methyl)piperazin-1-yl)methyl)-2-oxo-3-(2,2,2-trifluoroethyl)-2,3-dihydro-1H-benzo[d]imidazol-1-yl)-1-(4-methoxybenzyl) piperidine-2,6-dione (0.16 g).

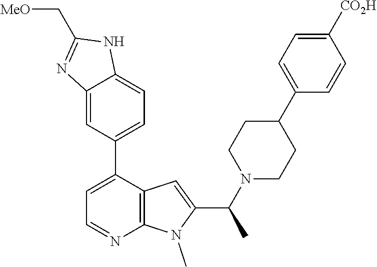 [SMILES: COCc1nc2cc(-c3ccnc4c3cc([C@H](C)N3CCC(c5ccc(C(=O)O)cc5)CC3)n4C)ccc2[nH]1]

A solution of Int. 3E308/3E309 (0.6 g) in DCM/4M HCl in dioxane (1:1; 12 mL) was stirred 2 h at 0° C. to RT and concentrated to give Int. 3E120 (S)-4-(1-(1-(4-(2-(methoxymethyl)-1H-benzo[d] imidazol-5-yl)-1-methyl-1H-(0.4 g, HCl salt).

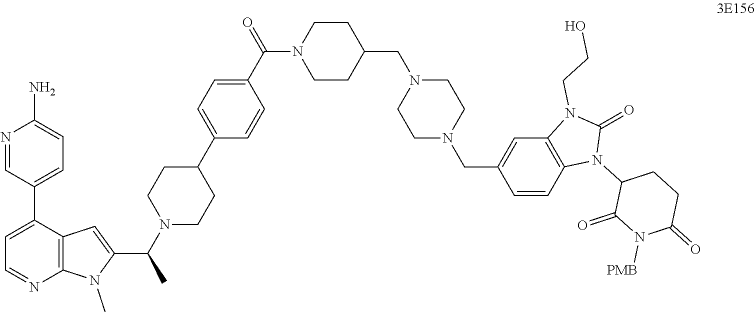

Ints. 3E157/3E83 (0.1 g/54 mg) and HATU (73 mg) were stirred ON in DMF/DIPEA (23:1, 3.1 mL) and diluted with water to precipitate Int. 3E156 3-(5-((4-((1-(4-(1-((S)-1-(4-(6-aminopyridin-3-yl)-1-methyl-1H-pyrrolo[2,3-b]pyridin-2-yl)ethyl)piperidin-4-yl)-benzoyl)piperidin-4-yl)methyl)piperazin-1-yl)methyl)-3-(2-hydroxyethyl)-2-oxo-2,3-dihydro-1H-benzo[d]imidazol-1-yl)-1-(4-methoxybenzyl) piperidine-2,6-dione (90 mg).

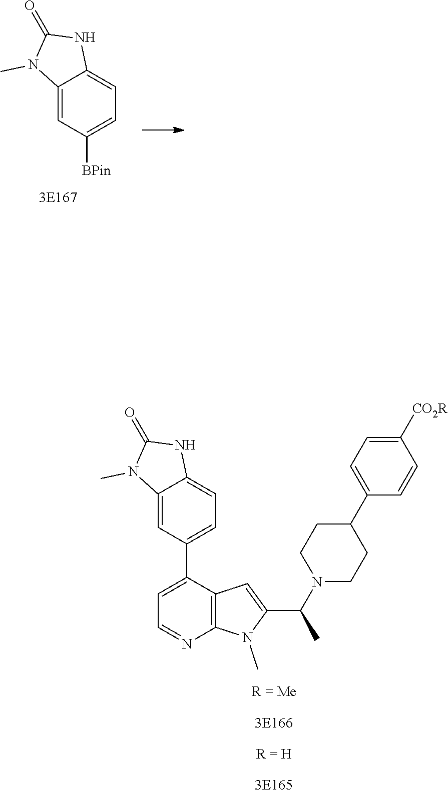

6-Bromo-1-methyl-1,3-dihydro-2H-benzo[d]imidazol-2-one (0.50 g), B₂Pin₂ (0.84 g), PdDPPFCl₂-DCM (0.18 g), and KOAc (0.43 g) were degassed in dioxane (10 mL) and stirred ON at 80° C. The OL (water/EtOAc) was dried, concentrated, and purified by FC (pentane/EtOAc 1:0 to 2:3) to give Int. 3E167 1-methyl-6-(4,4,5,5-tetramethyl-1,3,2-dioxaborolan-2-yl)-1,3-dihydro-2H-benzo[d] imidazol-2-one (0.60 g). Ints. 3E167/3E35 (0.36 g/0.40 g), PdDPPFCl₂-DCM (72 mg), and Na₂CO₃ (0.19 g) were degassed in dioxane/water (10:1; 5.5 mL) and stirred ON at 110° C., diluted with EtOAc, filtered, and concentrated. The residue was purified by FC (DCM/MeOH 1:0 to 73:7) to give Int. 3E166 methyl(S)-4-(1-(1-(1-methyl-4-(3-methyl-2-oxo-2,3-dihydro-1H-benzo[d]imidazol-5-yl)-1H-pyrrolo[2,3-b]pyridin-2-yl)ethyl)-piperidin-4-yl)benzoate (0.32 g). This material (0.3 g) and LiOH—H₂O (0.1 g) were stirred ON in MeOH/THF/water (2:2:1; 15 mL), concentrated, and triturated in dilute aq. HCl to give Int. 3E165 (S)-4-(1-(1-(1-methyl-4-(3-methyl-2-oxo-2,3-dihydro-1H-benzo[d]imidazol-5-yl)-1H-pyrrolo[2,3-b]pyridin-2-yl)ethyl)piperidin-4-yl)benzoic acid (0.22 g).

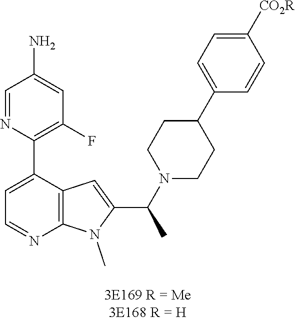

Int. 3E170 (0.5 g), 6-bromo-5-fluoro-pyridin-3-amine (0.19 g), K₃PO₄ (0.42 g), and PdDPPFCl₂-DCM (41 mg) were degassed in dioxane/water (10:1; 5.5 mL) and stirred 1 h at 100° C. under MW conditions. The OL (EtOAc/water) was dried, concentrated, and purified by FC (EtOAc) to give Int. 3E169 (0.3 g). 0.18 g of this material and LiOH—H₂O (45 mg) were stirred ON in MeOH/THF/water (10:5:3; 9 mL) at 0° C. to RT, concentrated, and triturated in dilute aq. citric acid to give Int. 3E168 4-[1-[(1S)-1-[4-(5-amino-3-fluoro-2-pyridyl)-1-methyl-pyrrolo[2,3-b]pyridin-2-yl]ethyl]-4-piperidyl]benzoic acid (0.16 g).

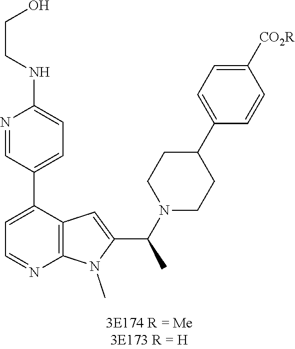

Int. 3E35 (0.30 g), 2-[5-(4,4,5,5-tetramethyl-1,3,2-dioxaborolan-2-yl)-2-pyridyl]-amino]ethanol (0.26 g), K₃PO₄ (0.28 g), and RuPhos Pd G2 (26 mg) were degassed in dioxane/water (9:1; 10 mL) and stirred ON at 80-85° C., diluted with EtOAc and filtered. The OL was dried, concentrated, and purified by FC (pentane/EtOAc 7:3 to 1:1) to give Int. 3E174 methyl(S)-4-(1-(1-(4-(6-((2-hydroxyethyl)-amino)-pyridin-3-yl)-1-methyl-1H-pyrrolo[2,3-b]-pyridin-2-yl)ethyl)piperidin-4-yl)benzoate (0.35 g). 0.31 g of this material and LiOH—H₂O (74 mg) were stirred ON in MeOH/THF/water (10:5:3; 9 mL), concentrated, and triturated in dilute aq. citric acid to give Int. 3E173 (S)-4-(1-(1-(4-(6-((2-hydroxyethyl)amino)pyridin-3-yl)-1-methyl-1H-pyrrolo[2,3-b]pyridin-2-yl)ethyl)piperidin-4-yl)benzoic acid (0.25 g).

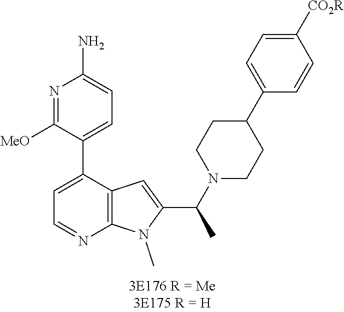

Int. 3E170 (0.50 g), 5-bromo-6-methoxy-pyridin-2-amine (0.24 g), K₃PO₄ (0.50 g), and PdDPPFCl₂-DCM (48 mg) were degassed in dioxane/water (9:1; 10 mL) and stirred 1 h at 100° C. The filtrate after filtration was diluted with EtOAc. The OL was dried, concentrated, and purified by FC (pentane/EtOAc 7:3 to 1:1) to give Int. 3E176 methyl 4-[1-[(1S)-1-[4-(6-amino-2-methoxy-3-pyridyl)-1-methyl-pyrrolo[2,3-b]pyridin-2-yl]ethyl]-4-piperidyl]benzoate (0.48 g). 0.40 g of this material and LiOH—H₂O (98 mg) were stirred ON in MeOH/THF/water (10; 5:3; 9 mL) at 0° C. to RT, concentrated, and triturated in dilute aq. citric acid to give Int. 3E175 (S)-4-(1-(1-(4-(6-amino-2-methoxypyridin-3-yl)-1-methyl-1H-pyrrolo[2,3-b]pyridin-2-yl)ethyl)piperidin-4-yl)benzoic acid (0.20 g).

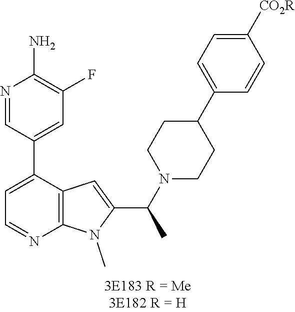

Int. 3E170 (0.30 g), 5-bromo-3-fluoropyridin-2-amine (0.11 mg), K₃PO₄ (0.25 g), and PdDPPFCl₂-DCM (24 mg) were degassed in dioxane/water (10:1; 3.3 mL) and stirred 1 h at 100° C. under MW conditions. The OL (EtOAc/water) was dried, concentrated, and purified by FC (EtOAc) to give Int. 3E183 methyl(S)-4-(1-(1-(4-(6-amino-5-fluoropyridin-3-yl)-1-methyl-1H-pyrrolo[2,3-b]pyridin-2-yl)ethyl)piperidin-4-yl)benzoate (0.25 g). Int. 3E182 (S)-4-(1-(1-(4-(6-amino-5-fluoropyridin-3-yl)-1-methyl-1H-pyrrolo[2,3-b]pyridin-2-yl)ethyl)piperidin-4-yl)benzoic acid (0.20 g) was prepared similarly to 3E179 from Int. 3E183 (0.42 g).

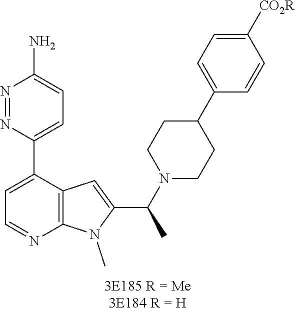

Int. 3E177 (0.44 g), 6-bromopyridazin-3-amine (0.18 g), Na₂CO₃ (0.33 g), and PdDPPFCl₂-DCM (84 mg) were degassed in dioxane/water (4:1; 8 mL) and stirred ON at 110° C., diluted with EtOAc and filtered. The OL was dried, concentrated, and purified by FC (pentane/EtOAc 1:0 to 2:3) to give Int. 3E185 methyl(S)-4-(1-(1-(4-(6-aminopyridazin-3-yl)-1-methyl-1H-pyrrolo[2,3-b]pyridin-2-yl)ethyl)piperidin-4-yl)benzoate (0.20 g). A solution of Int. 3E185 (0.20 g) in dioxane/6M aq. HCl (2:0.8, 2.8 mL) was stirred ON at 80° C. and concentrated to give Int. 3E184 (S)-4-(1-(1-(4-(6-aminopyridazin-3-yl)-1-methyl-1H-pyrrolo[2,3-b]pyridin-2-yl)ethyl)piperidin-4-yl)benzoic acid (0.20 g, HCl salt).

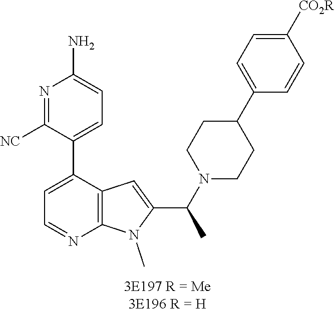

Int. 3E35 (0.25 g), 6-amino-3-(4,4,5,5-tetramethyl-1,3,2-dioxaborolan-2-yl) picolinonitrile (0.19 g), K₃PO₄ (0.23 g), and PdDPPFCl₂-DCM (40 mg) were degassed in dioxane/water (10:1; 3.3 mL) and stirred 2.5 h at 120° C. under MW conditions. The OL (water/EtOAc) was washed with brine, dried, concentrated, and purified by FC (EtOAc/pentane 3:7 to 3:2) to give Int. 3E197 methyl 4-[1-[(1S)-1-[4-(6-amino-2-cyano-3-pyridyl)-1-methyl-pyrrolo-[2,3-b]pyridin-2-yl]ethyl]-4-piperidyl]benzoate (0.2 g). Int. 3E197 (0.25 g) and LiOH—H₂O (64 mg) were stirred ON in MeOH/THF/water (5:5:2; 12 mL), concentrated, and triturated in dilute aq. citric acid to give Int. 3E196 (S)-4-(1-(1-(4-(6-amino-2-cyanopyridin-3-yl)-1-methyl-1H-pyrrolo[2,3-b]pyridin-2-yl)ethyl)piperidin-4-yl)benzoic acid (0.20 g).

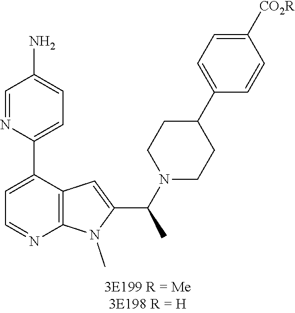

Int. 3E170 (0.50 g), 6-bromopyridin-3-amine (0.17 g), K₃PO₄ (0.42 g), and PdDPPFCl₂-DCM (77 mg) were degassed in dioxane/water (10:1; 5.5 mL) and stirred 2 h at 130° C. under MW conditions and filtered. The OL (water/EtOAc) was dried, concentrated, and purified by FC (pentane/EtOAc 7:3 to 1:1) to give Int. 3E199 methyl 4-[1-[(1S)-1-[4-(5-amino-2-pyridyl)-1-methyl-pyrrolo[2,3-b]pyridin-2-yl] ethyl]-4-piperidyl]benzoate (0.15 g). A mixture of Int. 3E199 (0.20 g) and LiOH—H₂O (87 mg) in MeOH/THF/water (10:5:2.5; 17.5 mL) were stirred ON, concentrated, and triturated in dilute aq. citric acid to precipitate Int. 3E198 (S)-4-(1-(1-(4-(5-aminopyridin-2-yl)-1-methyl-1H-pyrrolo[2,3-b]pyridin-2-yl)ethyl)piperidin-4-yl)benzoic acid (0.15 g).

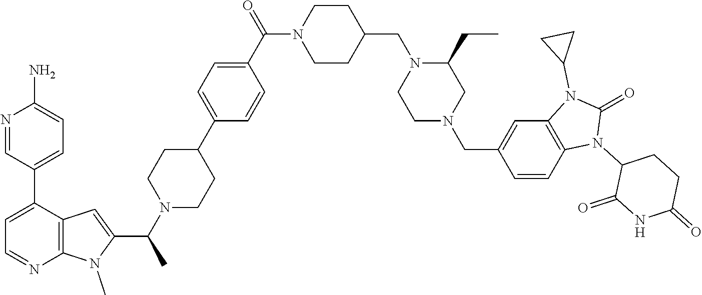

Ints. 3E209/3E68 (0.55 g/0.50 g) and HATU (0.13 g) were stirred ON in DMF/DIPEA (7.1:1, 5.7 mL) and diluted with water to precipitate Int. 3E208 3-(5-(((S)-4-((1-(4-(1-((S)-1-(4-(6-aminopyridin-3-yl)-1-methyl-1H-pyrrolo[2,3-b]pyridin-2-yl)ethyl)piperidin-4-yl)benzoyl)piperidin-4-yl)methyl)-3-ethylpiperazin-1-yl)methyl)-3-cyclopropyl-2-oxo-2,3-dihydro-1H-benzo[d]imidazol-1-yl) piperidine-2,6-dione (0.22 mg, after HPLC-purification).

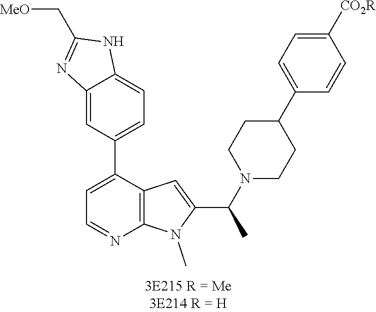

3E310/3E311 (0.77 g) was stirred ON in DCM/TFA (1.3:1, 19.3 mL), concentrated, and purified by FC (DCM/MeOH 1:0 to 3:2) to give Int. 3E215 methyl(S)-4-(1-(1-(4-(2-(methoxymethyl)-1H-benzo[d]-imidazol-5-yl)-1-methyl-1H-pyrrolo[2,3-b]pyridin-2-yl)ethyl)piperidin-4-yl)benzoate (0.60 g). This material was stirred in THF/MeOH/2M aq. NaOH (2:1:1; 12 mL) and HPLC-purified to give Int. 3E214 (S)-4-(1-(1-(4-(2-(methoxymethyl)-1H-benzo[d]imidazol-5-yl)-1-methyl-1H-pyrrolo [2,3-b] pyridin-2-yl)ethyl)piperidin-4-yl)benzoic acid (0.58 g).

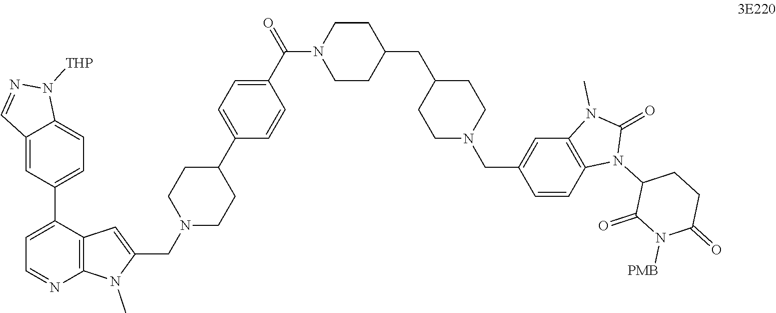

Int. 3A2 (20 mg) and HATU (18 mg) were stirred 0.3 h in DMF/DIPEA (67:1, 2 mL). Int. 3E221 (26 mg) was added and stirring continued ON. The OL (water/2MeTHF) was washed with sat. aq. NaHCO₃ and brine, dried, concentrated, and purified by FC (DCM/MeOH 1:0 to 9:1) to give Int. 3E220 1-(4-methoxybenzyl)-3-(3-methyl-5-((4-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)-piperidin-1-yl)methyl)-2-oxo-2,3-dihydro-1H-benzo[d]imidazol-1-yl) piperidine-2,6-dione (18 mg).

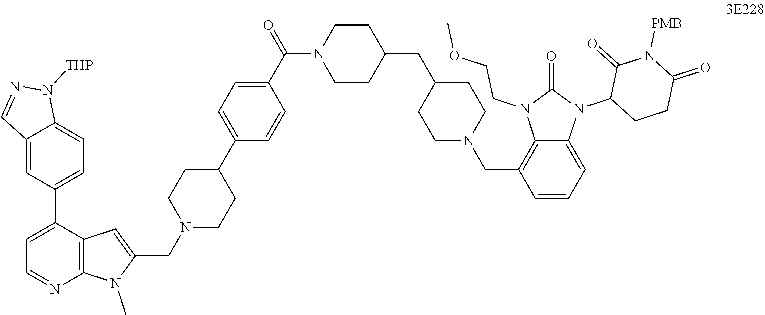

Ints. 3A2/3E229 (34 mg/30 mg) and HATU (24 mg) were stirred 2 h in DMF/DIPEA (24:1, 2.08 mL), filtered, and HPLC-purified to give Int. 3E228 1-(4-methoxybenzyl)-3-(3-(2-methoxyethyl)-4-((4-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)-piperidin-1-yl)methyl)-2-oxo-2,3-dihydro-1H-benzo[d] imidazol-1-yl) piperidine-2,6-dione (47 mg).

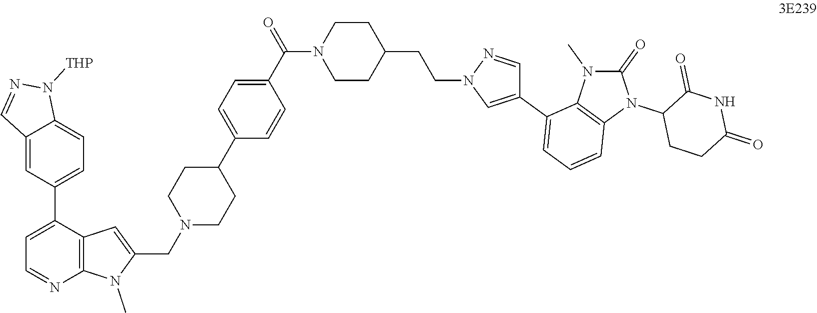

Int. 3E240 (17 mg, HCl salt), Int. 3A2 (20 mg) and HATU (14 mg) were stirred ON in ACN/DIPEA/DMF (10:1:1, 0.7 mL). The OL (aq. NaHCO₃/DCM) was washed with brine, dried, concentrated, and purified by FC (DCM/MeOH 97:3) to give Int. 3E239 3-[3-methyl-4-[1-[2-[1-[4-[1-[[1-methyl-4-(1-tetrahydropyran-2-ylindazol-5-yl) pyrrolo[2,3-b]pyridin-2-yl]methyl]-4-piperidyl]benzoyl]-4-piperidyl]ethyl]pyrazol-4-yl]-2-oxo-benzimidazol-1-yl]piperidine-2,6-dione (13 mg).

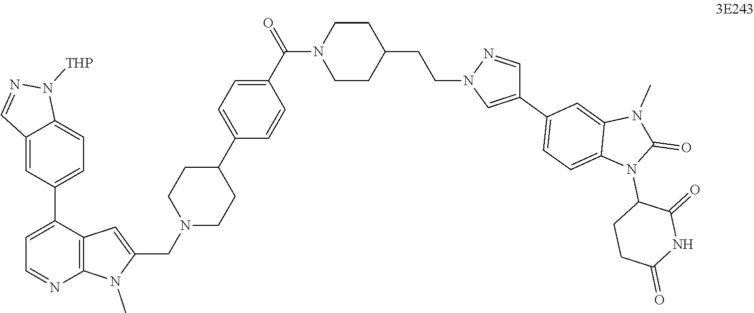

Int. 3E244 (26 mg, HCl salt), Int. 3A2 (31 mg) and HATU (22 mg) were stirred 1 h in DMF/DIPEA (2.5:1, 0.28 mL). The OL (aq. NaHCO₃/DCM) was washed with brine, dried, concentrated, and purified by FC (DCM/MeOH 97:3 to 96:4) to give Int. 3E243 3-(3-methyl-5-(1-(2-(1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)ethyl)-1H-pyrazol-4-yl)-2-oxo-2,3-dihydro-1H-benzo[d] imidazol-1-yl) piperidine-2,6-dione (29 mg).

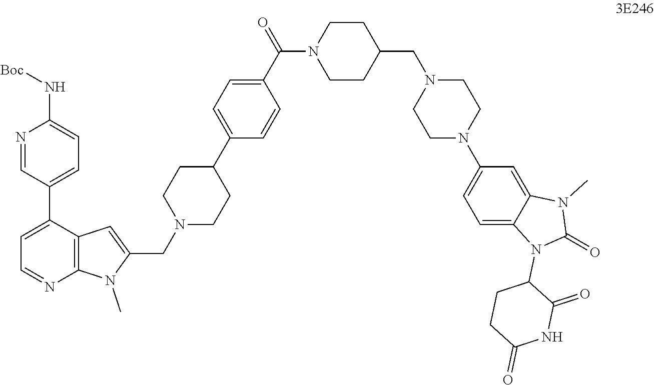 [SMILES: Cn1c(CN2CCC(c3ccc(C(=O)N4CCC(CN5CCN(c6ccc7c(c6)n(C)c(=O)n7C6CCC(=O)NC6=O)CC5)CC4)cc3)CC2)cc2c(-c3ccc(NC(=O)OC(C)(C)C)nc3)ccnc21]

Ints. 3E56/3E247 (35 mg/47 mg) and HATU (28 mg) were stirred ON in DMF/DIPEA (6.3:1, 1.2 mL). The OL (water/DCM) was washed with brine, dried, concentrated, and purified by FC (DCM/Et₃N/MeOH 95:5:0 to 85:5:10) to give Int. 3E246 tert-butyl (5-(2-((4-(4-(4-((4-(1-(2,6-dioxopiperidin-3-yl)-3-methyl-2-oxo-2,3-dihydro-1H-benzo[d]-imidazol-5-yl) piperazin-1-yl)methyl)piperidine-1-carbonyl)phenyl) piperidin-1-yl)methyl)-1-methyl-1H-pyrrolo[2,3-b]pyridin-4-yl)pyridin-2-yl) carbamate (53 mg).

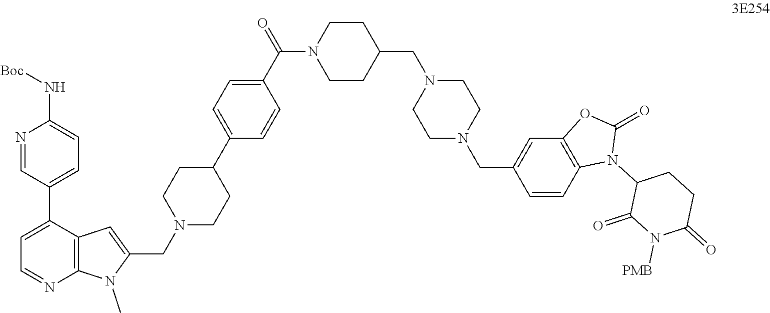

Ints. 3E56/3E255(43 mg/40 mg) and HATU (31 mg) were stirred ON in DMF/DIPEA (8.3:1, 1.1 mL. The OL (water/EtOAc) was dried, concentrated, and purified by FC (DCM/MeOH 1:0 to 95:5) to give Int. 3E254 tert-butyl (5-(2-((4-(4-(4-((4-((3-(1-(4-methoxybenzyl)-2,6-dioxopiperidin-3-yl)-2-oxo-2,3-dihydrobenzo[d]oxazol-6-yl)-methyl)piperazin-1-yl)methyl)piperidine-1-carbonyl)phenyl) piperidin-1-yl)methyl)-1-methyl-1H-pyrrolo[2,3-b]pyridin-4-yl)pyridin-2-yl) carbamate (48 mg).

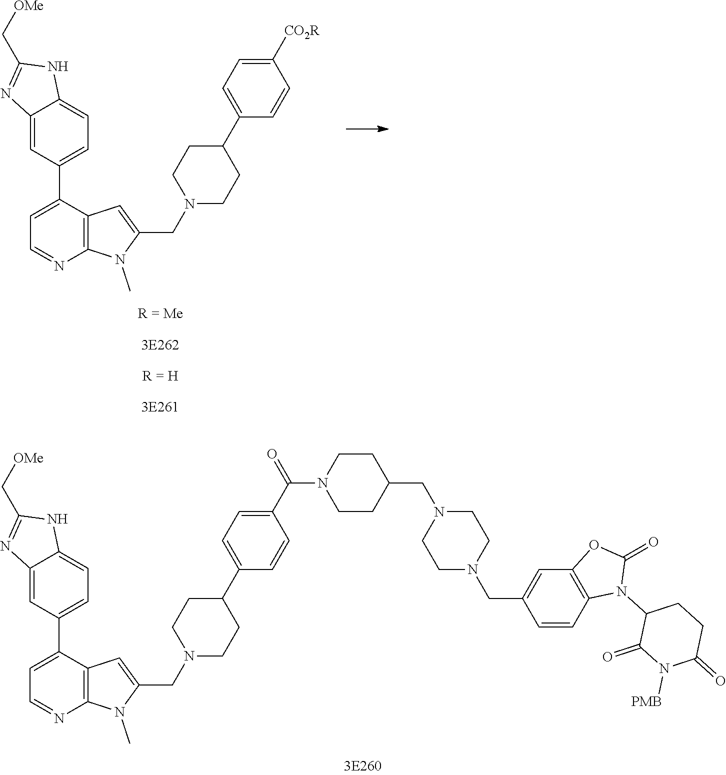

Ints. 1AC5/3E8 (1.2 g/1.1 g), PdDPPFCl₂-DCM (0.33 g), and NaHCO₃ (1.1 g) were degassed in dioxane/water (10:1; 22 mL) and stirred ON at 90° C., filtered, concentrated, and purified by FC (DCM/MeOH 9:1) and by HPLC to give Int. 3E262 methyl 4-(1-((4-(2-(methoxy-methyl)-1H-benzo[d] imidazol-5-yl)-1-methyl-1H-pyrrolo-[2,3-b]pyridin-2-yl)methyl)piperidin-4-yl)benzoate (0.58 g). A solution of Int. 3E262 (0.12 g) in THF/MeOH/2M aq. NaOH (2:1:0.04, 5 mL) was stirred ON and then acidified with aq. HCl to precipitate Int. 3E261 4-[1-[4-[2-(methoxymethyl)-1H-benzimidazol-5-yl]-1-methyl-pyrrolo[2,3-b]pyridin-2-yl]methyl]-4-piperidyl]benzoic acid (0.66 g). Ints. 3E255/3E261 (40 mg/42 mg) and HATU (31 mg) were stirred ON in DMF/DIPEA (1:0.06, 2.1 mL). The OL (water/EtOAc) was concentrated and purified by FC (DCM/MeOH 1:0 to 3:1) to give Int. 3E260 (48 mg).

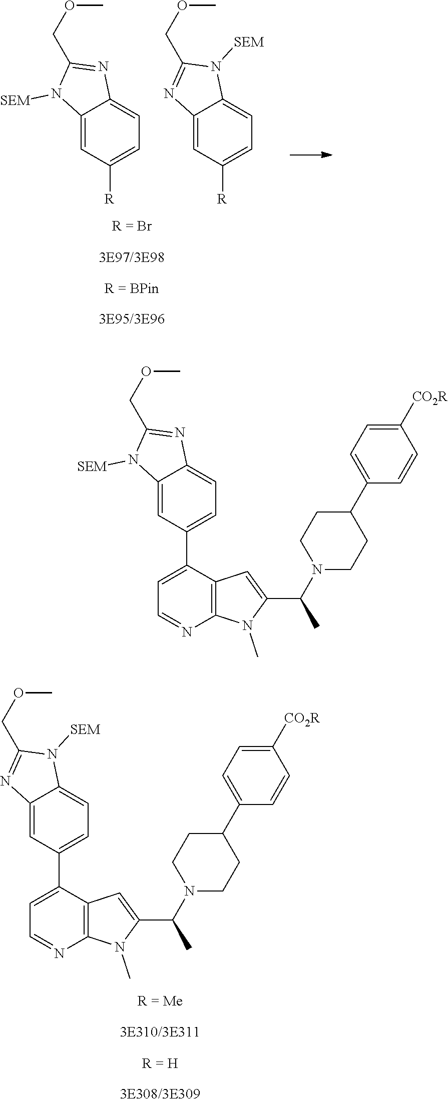

5-Bromo-2-(methoxymethyl)-1H-benzo[d]imidazole (25 g) was dissolved in DMF (150 mL). NaH (6.2 g) was added at 0° C. The mixture was stirred 0.25 h at 0° C. SEM-C1 (20.8 g) was added and stirring continued 0.25 h at 0° C. The OL (water/EtOAc) was dried and concentrated. The residue was combined with another batch prepared similarly and purified by FC (pentane/EtOAc 3:7) to give Int. 3E97/3E98 5-bromo-2-(methoxymethyl)-1-((2-(trimethylsilyl)-ethoxy)methyl)-1H-benzo[d]imidazole and 6-bromo-2-(methoxymethyl)-1-((2-(trimethyl-silyl) ethoxy)methyl)-1H-benzo[d]imidazole (30 g). 20 g of Int. 3E97/3E98, B₂Pin₂ (15 g), KOAc (15.8 g), and PdDPPFCl₂-DCM (2.2 g) were degassed in dioxane (100 mL) and stirred ON at 90° C., filtered, and concentrated. The residue was combined with another batch prepared in similarly on 10 g scale and purified by FC (pentane/EtOAc 1:4) to give Int. 3E95/3E96 2-(methoxymethyl)-6-(4,4,5,5-tetramethyl-1,3,2-dioxaborolan-2-yl)-1-((2-(trimethylsilyl) ethoxy)methyl)-1H-benzo[d]imidazole and 2-(methoxymethyl)-5-(4,4,5,5-tetramethyl-1,3,2-dioxaborolan-2-yl)-1-((2-(trimethylsilyl) ethoxy)methyl)-1H-benzo[d]imidazole (20 g). Ints. 3E35 (2.0 g) and 3E95/3E96 (2.0 g), NaHCO₃ (0.74 g), and PdDPPFCl₂-DCM (0.36 g) were degassed in dioxane/water (4.5:1, 22 mL) and stirred ON at 80° C. The OL (water/EtOAc) was washed with brine, dried, concentrated, and purified by FC (hexane/EtOAc 4:1 to 7:3) to give Int. 3E310/3E311 methyl(S)-4-(1-(1-(4-(2-(methoxymethyl)-1-((2-(trimethyl-silyl) ethoxy)methyl)-1H-benzo[d]imidazol-5-yl)-1-methyl-1H-pyrrolo[2,3-b]pyridin-2-yl)ethyl)piperidin-4-yl)benzoate and methyl(S)-4-(1-(1-(4-(2-(methoxymethyl)-1-((2-(trimethylsilyl)-ethoxy)methyl)-1H-benzo[d]-imidazol-6-yl)-1-methyl-1H-pyrrolo[2,3-b]pyridin-2-yl)ethyl)piperidin-4-yl)benzoate (2.7 g). This material and LiOH—H₂O (0.51 g) were stirred ON in THF/MeOH/water (2:2:1; 50 mL), concentrated, and triturated in dilute aq. citric acid to precipitate Int. 3E308/3E309 (S)-4-(1-(1-(4-(2-(methoxy-methyl)-1-((2-(trimethylsilyl) ethoxy)-methyl)-1H-benzo[d]imidazol-5-yl)-1-methyl-1H-pyrrolo[2,3-b]pyridin-2-yl)ethyl)piperidin-4-yl)benzoic acid and(S)-4-(1-(1-(4-(2-(methoxymethyl)-1-((2-(trimethylsilyl) ethoxy)methyl)-1H-benzo[d] imidazol-6-yl)-1-methyl-1H-pyrrolo[2,3-b]pyridin-2-yl)ethyl)piperidin-4-yl)benzoic acid (1.56 g).

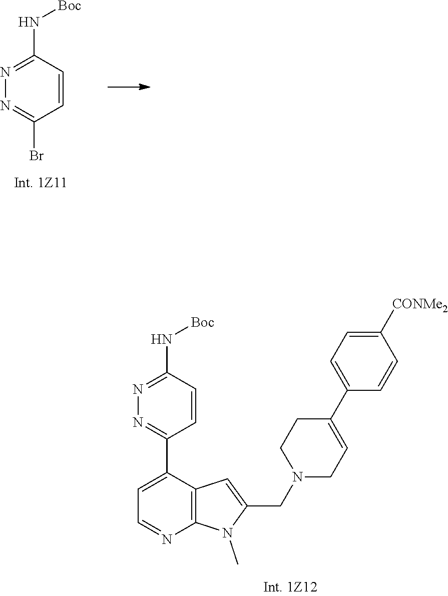

Ints. 1Z11/1Z10 (0.43 g/0.72 g), K₂CO₃ (0.60 g), and [2-(di-1-adamantyl-phosphino)-2′, 4′, 6′-triisopropyl-3,6-dimethoxybiphenyl] [2-(2′-amino-1,1′-biphenyl)]palladium(II) methanesulfonate (29 mg) were stirred 48 h in dioxane (20 mL) at 90° C. under an inert atmosphere. The OL (water/MTBE) was concentrated to give Int. 1Z12 tert-butyl (6-(2-((4-(4-(dimethylcarbamoyl)phenyl)-3,6-dihydropyridin-1(2H)-yl)methyl)-1-methyl-1H-pyrrolo[2,3-b]pyridin-4-yl)pyridazin-3-yl) carbamate (0.75 g).

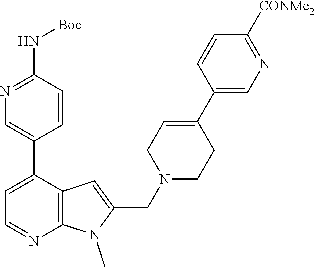

PdDPPFCl₂-DCM (16 mg), Int. 1Z50 (0.4 g), tert-butyl (5-(4,4,5,5-tetramethyl-1,3,2-dioxaborolan-2-yl)pyridin-2-yl) carbamate (0.3 g), and K₂CO₃ (0.4 g) were stirred ON in dioxane/H₂O (5:1, 6 mL) at 100° C. under an inert atmosphere. The mixture was concentrated to give Int. 1Z13 tert-butyl (5-(2-((6-(dimethylcarbamoyl)-3′, 6′-dihydro-[3,4′-bipyridin]-1′ (2′H)-yl)methyl)-1-methyl-1H-pyrrolo[2,3-b]pyridin-4-yl)pyridin-2-yl) carbamate (0.35 g).

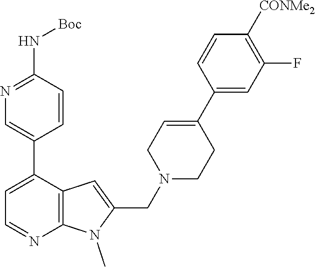

PdDPPFCl₂-DCM (4 mg), Int. 1Z54 (0.25 g), tert-butyl (5-(4,4,5,5-tetramethyl-1,3,2-dioxaborolan-2-yl)pyridin-2-yl) carbamate (0.19 g), and K₂CO₃ (0.22 g) were stirred ON in dioxane/H₂O (5:1, 6 mL) at 100° C. under an inert atmosphere. The mixture was filtered and concentrated to give Int. 1Z21 tert-butyl (5-(2-((4-(4-(dimethylcarbamoyl)-3-fluorophenyl)-3,6-dihydropyridin-1(2H)-yl)methyl)-1-methyl-1H-pyrrolo[2,3-b]pyridin-4-yl)pyridin-2-yl) carbamate (0.27 g).

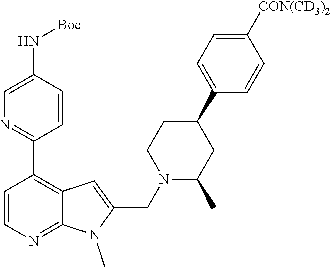

Ints. 1Q4/1D17 (0.20 g/0.14 g) and KI (9 mg) were stirred 16 h in DMF/DIPEA (4:1, 2.5 mL). The OL (water/EtOAc) was dried, filtered, concentrated, and purified by FC (DCM/MeOH 19:1) to give Int. 1Z22 tert-butyl (6-(2-(((2R,4R)-4-(4-(bis (methyl-d3) carbamoyl)phenyl)-2-methylpiperidin-1-yl)methyl)-1-methyl-1H-pyrrolo[2,3-b]pyridin-4-yl)pyridin-3-yl) carbamate (0.12 g).

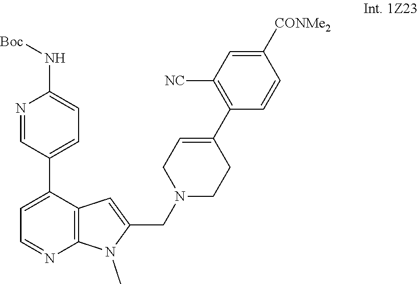

PdDPPFCl₂-DCM (1 mg), tert-butyl (5-(4,4,5,5-tetra-methyl-1,3,2-dioxaborolan-2-yl)pyridin-2-yl) carbamate (44 mg), Int. 1Z27 (60 mg), and K₂CO₃ (52 mg) were stirred ON in dioxane/H₂O (5:1, 6 mL) at 100° C. under an inert atmosphere. The mixture was concentrated to give Int. 1Z23 tert-butyl (5-(2-((4-(2-cyano-4-(dimethylcarbamoyl)-phenyl)-3,6-dihydropyridin-1(2H)-yl)methyl)-1-methyl-1H-pyrrolo[2,3-b]pyridin-4-yl)pyridin-2-yl) carbamate (60 mg).

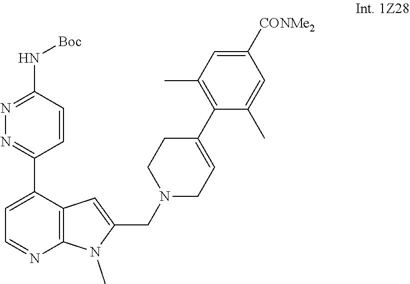

PdDPPFCl₂-DCM (50 mg), Ints. 1Z11/1Z30 (0.2 g/0.3 g), and K₂CO₃ (0.5 g) were stirred 12 h in dioxane (5:1, 18 mL) at 80° C. under an inert atmosphere. The OL (water/EtOAc) was concentrated to give Int. 1Z28 tert-butyl (6-(2-((4-(4-(dimethylcarbamoyl)-2,6-dimethylphenyl)-3,6-dihydropyridin-1(2H)-yl)methyl)-1-methyl-1H-pyrrolo[2,3-b]pyridin-4-yl)pyridazin-3-yl) carbamate (0.3 g).

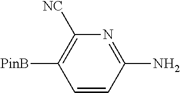

6-Amino-3-bromopicolinonitrile (10 g), bis (pinacolato)diboron (26 g), PdDPPFCl₂-DCM (4.1 g), and KOAc (15 g) were stirred 0.5 h in THF (200 mL) at 90° C. The mixture was filtered through celite. The OL (water/EtOAc) was dried, filtered, concentrated, and washed with Et₂O. The residue was purified by FC (hexane/EtOAc 5:4), concentrated, and washed with hexane to give Int. 1Z32 6-amino-3-(4,4,5,5-tetramethyl-1,3,2-dioxaborolan-2-yl) picolinonitrile (5.2 g).

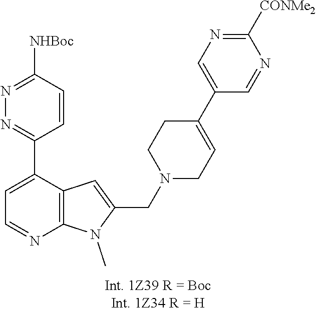

Int. 1Z38 (90 mg), 6-bromo-N,N-dimethylpyridazin-3-amine (40 mg), K₂CO₃ (75 mg), and PdDPPFCl₂-DCM (1 mg) were stirred ON in dioxane/H₂O (20:1, 21 mL) at 100° C. under an inert atmosphere. The mixture was filtrated, and concentrated to give Int. 1Z39 5-(1-((4-(6-(dimethylamino)pyridazin-3-yl)-1-methyl-1H-pyrrolo[2,3-b]pyridin-2-yl)methyl)-1,2,3,6-tetrahydropyridin-4-yl)-N,N-dimethylpyrimidine-2-carboxamide (75 mg). Ints. 1Z36/1Z37 (0.72 g/1.2 g), K₂CO₃ (0.99 g), and [2-(di-1-adamantylphosphino)-2′, 4′, 6′-triisopropyl-3,6-dimethoxybiphenyl] [2-(2′-amino-1,1′-biphenyl)]palladium(II) methanesulfonate (48 mg) were stirred 48 h in dioxane (20 mL) at 90° C. The OL (water/MTBE) was concentrated to give Int. 1Z34 tert-butyl (6-(2-((4-(4-(dimethylcarbamoyl)phenyl) piperidin-1-yl)methyl)-1-methyl-1H-pyrrolo[2,3-b]pyridin-4-yl)pyridazin-3-yl) carbamate (0.5 g).

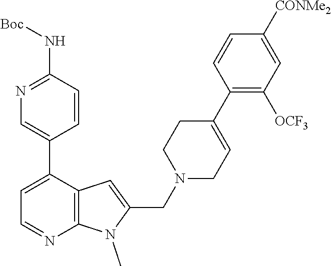

PdDPPFCl₂-DCM (1 mg), Int. 1Z136 (60 mg), tert-butyl 4-(4,4,5,5-tetramethyl-1,3,2-dioxaborolan-2-yl)-3,6-dihydropyridine-1(2H)-carboxylate (39 mg), and K₂CO₃ (46 mg) were stirred ON in dioxane/H₂O (5:1, 6 mL) at 100° C. under an inert atmosphere. The mixture was concentrated to give Int. 1Z49 tert-butyl (5-(2-((4-(4-(dimethylcarbamoyl)-2-(trifluoromethoxy)phenyl)-3,6-dihydropyridin-1(2H)-yl)methyl)-1-methyl-1H-pyrrolo[2,3-b]pyridin-4-yl)pyridin-2-yl) carbamate (60 mg).

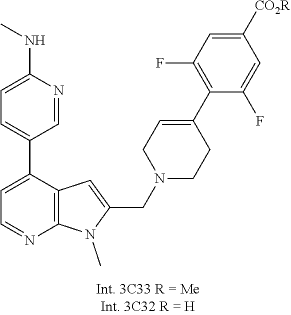

PdDPPFCl₂-DCM (0.17 g), Int. 3C31 (1 g), N-methyl-5-(4,4,5,5-tetramethyl-1,3,2-dioxaborolan-2-yl)-pyridin-2-amine (0.59 g), and NaHCO₃ (0.53 g) were stirred 3 h in dioxane/H₂O (5:1, 24 mL) at 75° C. under an inert atmosphere. The OL (water/EtOAc) was dried, filtered, concentrated, and purified by FC (PE/EtOAc 2:1 to 3:2) to give Int. 3C33 methyl 3,5-difluoro-4-(1-((1-methyl-4-(6-(methylamino)-pyridin-3-yl)-1H-pyrrolo[2,3-b]pyridin-2-yl)methyl)-1,2,3,6-tetrahydropyridin-4-yl)benzoate (0.80 g). LiOH·H₂O (0.13 g) and Int. 3C33 (0.80 g) were stirred 12 h in THF/H₂O (5:1, 22 mL). The residue after concentration was diluted with aq. citric acid to precipitate Int. 3C32 3,5-difluoro-4-(1-((1-methyl-4-(6-(methylamino)-pyridin-3-yl)-1H-pyrrolo[2,3-b]pyridin-2-yl)methyl)-1,2,3,6-tetrahydropyridin-4-yl)benzoic acid (0.60 g).

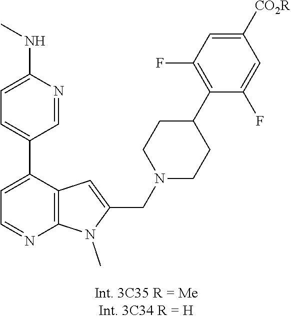

PdDPPFCl₂-DCM (0.17 g), Int. 3C15 (1.1 g), N-methyl-5-(4,4,5,5-tetramethyl-1,3,2-dioxaborolan-2-yl)pyridin-2-amine (0.70 g), and NaHCO₃ (0.58 g) were stirred 16 h in dioxane/H₂O (3:1, 20 mL) at 90° C. under an inert atmosphere. The mixture was filtered. The OL (water/EtOAc) was dried, filtered, concentrated, and purified by FC (PE/EtOAc 2:1 to 3:2) to give Int. 3C35 methyl 3,5-difluoro-4-(1-((1-methyl-4-(6-(methylamino)pyridin-3-yl)-1H-pyrrolo[2,3-b]pyridin-2-yl)methyl)piperidin-4-yl)benzoate (0.90 g). LiOH H₂O (0.49 g) and Int. 3C35 (1.2 g) were stirred 4 h in THF/MeOH/H₂O (10:3:2, 15 mL) at 0° C. to RT. The residue after concentration was diluted with aq. citric acid to precipitate Int. 3C34 3,5-difluoro-4-(1-((1-methyl-4-(6-(methylamino)pyridin-3-yl)-1H-pyrrolo [2,3-b] pyridin-2-yl)methyl)piperidin-4-yl)benzoic acid (1.0 g).

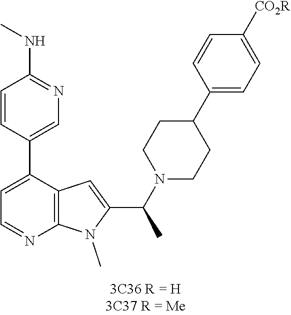

Int. 3E35 (3. 0 g), N-methyl-5-(4,4,5,5-tetramethyl-1,3,2-dioxaborolan-2-yl)pyridin-2-amine (1.8 g), NaHCO₃ (1.1 g) and PdDPPFCl₂-DCM (0.27 g) were stirred 16 h in dioxane/H₂O (4:1, 30 mL) at 85° C. under an inert atmosphere. The OL (water/EtOAc) was dried, filtered, concentrated, and purified by FC (PE/EtOAc 1:1 to 2:3) to give Int. 3C37 methyl(S)-4-(1-(1-(1-methyl-4-(6-(methylamino)-pyridin-3-yl)-1H-pyrrolo[2,3-b]pyridin-2-yl)ethyl)piperidin-4-yl)benzoate (3.0 g). LiOH H₂O (0.89 g) and Int. 3C37 (2.1 g) were stirred 16 h in THF/MeOH/H₂O (2:2:1, 10 mL) at 0° C. The residue after concentration was stirred in aq. citric acid to precipitate Int. 3C36 (S)-4-(1-(1-(1-methyl-4-(6-(methylamino)pyridin-3-yl)-1H-pyrrolo[2,3-b]pyridin-2-yl)ethyl)piperidin-4-yl)benzoic acid (1.9 g).

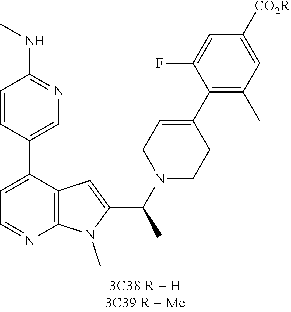

PdDPPFCl₂-DCM (84 mg), Int. 1AF86 (1.0 g), N-methyl-5-(4,4,5,5-tetra-methyl-1,3,2-dioxaborolan-2-yl)-pyridin-2-amine (0.63 g), and NaHCO₃ (0.60 g) were stirred 16 h in dioxane/H₂O (4:1, 10 mL) at 80° C. under an inert atmosphere. The mixture was filtered. The OL (water/EtOAc) was dried, filtered, and concentrated to give Int. 3C39 methyl(S)-3-fluoro-5-methyl-4-(1-(1-(1-methyl-4-(6-(methyl-amino)-pyridin-3-yl)-1H-pyrrolo[2,3-b]pyridin-2-yl)ethyl)-1,2,3,6-tetrahydro-pyridin-4-yl)-benzoate (1.2 g). LiOH H₂O (0.38 g) and Int. 3C39 (0.95 g) were stirred 6 h in THF/MeOH/H₂O (2:2:1, 25 mL) at 0° C. to 60° C. The residue after concentration was stirred in aq. HCl to precipitate Int. 3C38 (S)-3-fluoro-5-methyl-4-(1-(1-(1-methyl-4-(6-(methylamino)pyridin-3-yl)-1H-pyrrolo[2,3-b]pyridin-2-yl)ethyl)-1,2,3,6-tetrahydro-pyridin-4-yl)benzoic acid (0.80 g).

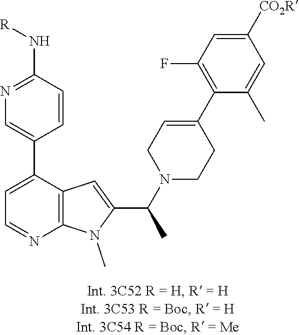

Int. 1AF86 (1.0 g), tert-butyl (5-(4,4,5,5-tetramethyl-1,3,2-dioxaborolan-2-yl)pyridin-2-yl) carbamate (0.72 g), K₃PO₄ (1.7 g), and PdDPPFCl₂-DCM (0.17 g) were stirred 16 h in dioxane/H₂O (5:1, 12 mL) at 90° C. under an inert atmosphere. The mixture was filtered. The OL (water/EtOAc) was dried, filtered, concentrated, and purified by FC (hexane/EtOAc 1:1) to give Int. 3C54 methyl(S)-4-(1-(1-(4-(6-((tert-butoxycarbonyl)amino)pyridin-3-yl)-1-methyl-1H-pyrrolo[2,3-b]pyridin-2-yl)ethyl)-1,2,3,6-tetrahydro-pyridin-4-yl)-3-fluoro-5-methylbenzoate (0.90 g). LiOH (0.32 g) was added to a solution of Int. 3C54 (0.90 g) in THF/H₂O (3:2, 9.5 mL) at 0° C. and stirred 16 h at 60° C. The residue after concentration was stirred in aq. citric acid to precipitate Int. 3C53 (S)-4-(1-(1-(4-(6-((tert-butoxycarbonyl)amino)-pyridin-3-yl)-1-methyl-1H-pyrrolo[2,3-b]pyridin-2-yl)ethyl)-1,2,3,6-tetrahydropyridin-4-yl)-3-fluoro-5-methylbenzoic acid (0.85 g). Int. 3C53 (0.90 g) was stirred 16 h in DCM/4M HCl in dioxane (2:1, 17 mL), neutralized, and concentrated to give Int. 3C52 (S)-4-(1-(1-(4-(6-aminopyridin-3-yl)-1-methyl-1H-pyrrolo[2,3-b]pyridin-2-yl)ethyl)-1,2,3,6-tetrahydropyridin-4-yl)-3-fluoro-5-methylbenzoic acid (0.25 g)

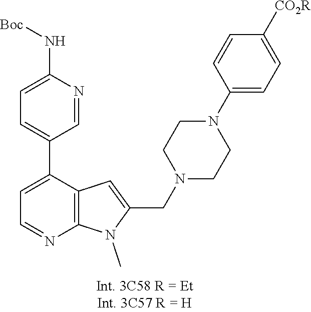

PdDPPFCl₂-DCM (0.20 g) was added to a mixture of Int. 3C59 (2.2 g), tert-butyl (5-(4,4,5,5-tetramethyl-1,3,2-dioxaborolan-2-yl)pyridin-2-yl) carbamate (2.0 g) and NaHCO₃ (0.81 g) in dioxane/H₂O (3:1, 24 mL) and stirred for 16 h at 85° C. under an inert atmosphere and filtered. The OL (water/EtOAc) was dried, filtered, concentrated and purified by FC (PE/EtOAc 2:1 to 1:1) to give Int. 3C58 ethyl 4-(4-((4-(6-((tert-butoxycarbonyl)amino)pyridin-3-yl)-1-methyl-1H-pyrrolo[2,3-b]pyridin-2-yl)methyl)piperazin-1-yl)-benzoate (2 g). 6M HCl (20 mL) was added drop wise at 0° C. to a solution of Int. 3C58 (2.1 g) in dioxane (15 mL) and stirred for 6 h at 85° C. The mixture was concentrated and triturated in Et₂O to give Int. 3C57 4-(4-((4-(6-aminopyridin-3-yl)-1-methyl-1H-pyrrolo[2,3-b]pyridin-2-yl)methyl)piperazin-1-yl)benzoic acid (1.5 g).

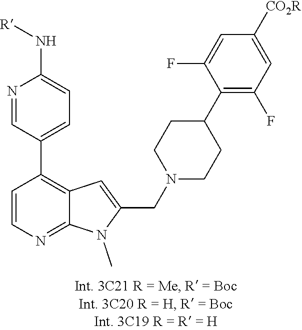

PdDPPFCl₂-DCM (0.15 g), NaHCO₃ (0.47 g), Int. 3C15 (0.90 g), tert-butyl (5-(4,4,5,5-tetramethyl-1,3,2-dioxaborolan-2-yl)pyridin-2-yl) carbamate (0.78 g) were stirred 16 h in dioxane/H₂O (5:1, 24 mL) at 90° C. under an inert atmosphere. The mixture was filtered. The OL (water/EtOAc) was dried, filtered, concentrated, and purified by FC (PE/EtOAc 1:0 to 2:1) to give Int. 3C21 methyl 4-(1-((4-(6-((tert-butoxycarbonyl)amino)pyridin-3-yl)-1-methyl-1H-pyrrolo[2,3-b]pyridin-2-yl)methyl)piperidin-4-yl)-3,5-difluorobenzoate (0.75 g). LiOH H₂O (0.21 g) and Int. 3C21 (0.75 g) were stirred 16 h in THF/H₂O (5:1, 24 mL) at 60° C. The mixture was diluted with aq. citric acid to precipitate Int. 3C20 4-(1-((4-(6-((tert-butoxycarbonyl)amino)pyridin-3-yl)-1-methyl-1H-pyrrolo[2,3-b]pyridin-2-yl)methyl)piperidin-4-yl)-3,5-difluorobenzoic acid (0.67 g). Int. 3C20 (0.65 g) was stirred 6 h in DCM/4M HCl in dioxane (3:2, 17 mL) at 0° C. to RT, concentrated and washed with Et₂O to give Int. 3C19 4-(1-((4-(6-aminopyridin-3-yl)-1-methyl-1H-pyrrolo[2,3-b]pyridin-2-yl)methyl)piperidin-4-yl)-3,5-difluorobenzoic acid (0.50 g, HCl salt).

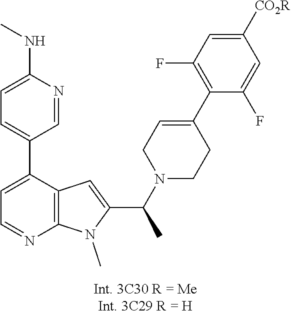

PdDPPFCl₂-DCM (83 mg), Int. 3C28 (0.50 g), N-methyl-5-(4,4,5,5-tetramethyl-1,3,2-dioxaborolan-2-yl)pyridin-2-amine (0.31 g), and NaHCO₃ (0.26 g) were stirred 16 h in dioxane/H₂O (5:1, 6 mL) at 80° C. under an inert atmosphere. The OL (water/EtOAc) was dried, filtered, concentrated, and purified by FC (PE/EtOAc 2:1 to 3:2) to give Int. 3C30 methyl(S)-3,5-difluoro-4-(1-(1-(1-methyl-4-(6-(methyl-amino)pyridin-3-yl)-1H-pyrrolo[2,3-b]pyridin-2-yl)ethyl)-1,2,3,6-tetrahydropyridin-4-yl)-benzoate (0.30 g). LiOH H₂O (73 mg) and Int. 3C30 (0.30 g) were stirred 6 h in THF/H₂O (3:1, 4 mL) at 60° C. The residue after concentration was diluted with aq. citric acid to precipitate Int. 3C29 (S)-3,5-difluoro-4-(1-(1-(1-methyl-4-(6-(methylamino)pyridin-3-yl)-1H-pyrrolo[2,3-b]pyridin-2-yl)ethyl)-1,2,3,6-tetrahydropyridin-4-yl)benzoic acid (0.20 g).

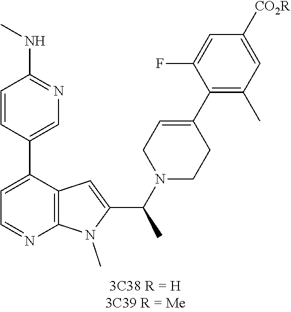

### Int. 3C38. (S)-3-fluoro-5-methyl-4-(1-(1-(1-methyl-4-(6-(methylamino)pyridin-3-yl)-1H-pyrrolo[2,3-b]pyridin-2-yl)ethyl)-1,2,3,6-tetrahydropyridin-4-yl)benzoic acid

PdDPPFCl₂-DCM (84 mg), Int. 1AF86 (1.0 g), N-methyl-5-(4,4,5,5-tetra-methyl-1,3,2-dioxaborolan-2-yl)pyridin-2-amine (0.63 g), and NaHCO₃ (0.60 g) were stirred 16 h in dioxane/H₂O (4:1, 10 mL) at 80° C. under an inert atmosphere. The mixture was filtered. The OL (water/EtOAc) was dried, filtered, and concentrated to give Int. 3C39 methyl(S)-3-fluoro-5-methyl-4-(1-(1-(1-methyl-4-(6-(methyl-amino)pyridin-3-yl)-1H-pyrrolo[2,3-b]pyridin-2-yl)ethyl)-1,2,3,6-tetrahydro-pyridin-4-yl)-benzoate (1.2 g). LiOH H₂O (0.38 g) and Int. 3C39 (0.95 g) were stirred 6 h in THF/MeOH/H₂O (2:2:1, 25 mL) at 0° C. to 60° C. The residue after concentration was stirred in aq. HCl to precipitate Int. 3C38 (0.80 g).

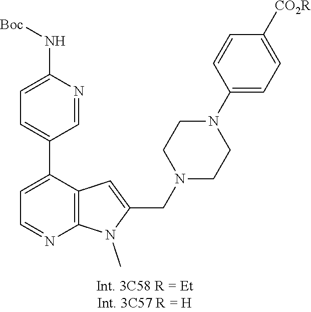

PdDPPFCl₂-DCM (0.20 g) was added to a mixture of Int. 3C59 (2.2 g), tert-butyl (5-(4,4,5,5-tetramethyl-1,3,2-dioxaborolan-2-yl)pyridin-2-yl) carbamate (2.0 g) and NaHCO₃ (0.81 g) in dioxane/H₂O (3:1, 24 mL) and stirred for 16 h at 85° C. under an inert atmosphere and filtered. The OL (water/EtOAc) was dried, filtered, concentrated and purified by FC (PE/EtOAc 2:1 to 1:1) to give Int. 3C58 ethyl 4-(4-((4-(6-((tert-butoxycarbonyl)amino)pyridin-3-yl)-1-methyl-1H-pyrrolo[2,3-b]pyridin-2-yl)methyl)piperazin-1-yl)benzoate (2 g). 6M HCl (20 mL) was added drop-wise at 0° C. to a solution of Int. 3C58 (2.1 g) in dioxane (15 mL) and stirred for 6 h at 85° C. The mixture was concentrated and triturated in Et₂O to give Int. 3C57 (1.5 g).

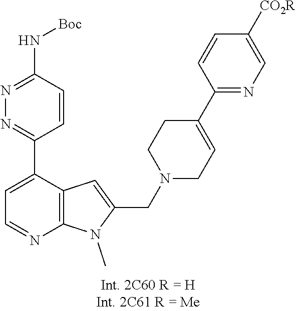

Int. 2C62 (0.30 g), tert-butyl (6-bromopyridazin-3-yl) carbamate (0.14 g), K₂CO₃ (0.19 g) and PdDPPFCl₂-DCM (37 mg) were stirred ON in dioxane/water (4:1, 10 mL) at 80° C. under an inert atmosphere. The OL (water/EtOAc) was dried, filtered, concentrated, and HPLC-purified to give Int. 2C61 methyl 1′-((4-(6-((tert-butoxycarbonyl)-amino)-pyridazin-3-yl)-1-methyl-1H-pyrrolo [2,3-b] pyridin-2-yl)methyl)-1′, 2′, 3′, 6′-tetrahydro-[2,4′-bipyridine]-5-carboxylate (0.20 g). LiOH H₂O (19 mg) and Int. 2C61 (25 mg) were stirred ON in THF/H₂O (1:1, 4 mL). Et₃N·HCl (92 mg) was added. The mixture was concentrated to give Int. 2C60 1′-((4-(6-((tert-butoxycarbonyl)amino)pyridazin-3-yl)-1. methyl-1H-pyrrolo[2,3-b]pyridin-2-yl)methyl)-1′, 2′, 3′, 6′-tetrahydro-[2,4′-bipyridine]-5-carboxylic acid (24 mg).

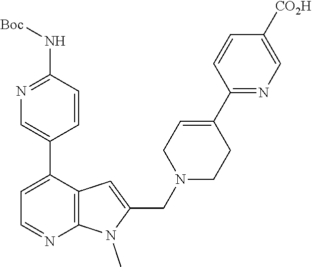 [SMILES: Cn1c(CN2CC=C(c3ccc(C(=O)O)cn3)CC2)cc2c(-c3ccc(NC(=O)OC(C)(C)C)nc3)ccnc21]

Int. 2C68 (285 mg), tert-butyl (5-(4,4,5,5-tetramethyl-1,3,2-dioxaborolan-2-yl)pyridin-2-yl) carbamate (227 mg), K₂CO₃ (268 mg) and PdDPPFCl₂-DCM (53 mg) were stirred ON in dioxane/H₂O (4:1, 10 mL) at 80° C. under an inert atmosphere. The OL (water/EtOAc) was dried, filtered, concentrated, and HPLC-purified to give Int. 2C67 methyl 1′-((4-(6-((tert-butoxycarbonyl)amino)pyridin-3-yl)-1-methyl-1H-pyrrolo[2,3-b]pyridin-2-yl)methyl)-1′, 2′, 3′, 6′-tetrahydro-[2,4′-bipyridine]-5-carboxylate (57 mg). Int. 2C66 1′-((4-(6-((tert-butoxycarbonyl)amino)pyridin-3-yl)-1-methyl-1H-pyrrolo[2,3-b]pyridin-2-yl)methyl)-1′, 2′, 3′, 6′-tetrahydro-[2,4′-bipyridine]-5-carboxylic acid (19 mg) was prepared similarly to Int. 2C60 from Int. 2C67 (20 mg).

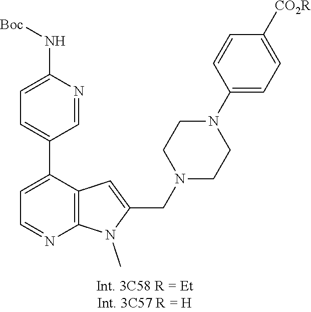

PdDPPFCl₂-DCM (0.20 g) was added to a mixture of Int. 3C59 (2.2 g), tert-butyl (5-(4,4,5,5-tetramethyl-1,3,2-dioxaborolan-2-yl)pyridin-2-yl) carbamate (2.0 g) and NaHCO₃ (0.81 g) in dioxane/H₂O (3:1, 24 mL) and stirred for 16 h at 85° C. under an inert atmosphere and filtered. The OL (water/EtOAc) was dried, filtered, concentrated and purified by FC (PE/EtOAc 2:1 to 1:1) to give Int. 3C58 ethyl 4-(4-((4-(6-((tert-butoxycarbonyl)amino)pyridin-3-yl)-1-methyl-1H-pyrrolo[2,3-b]pyridin-2-yl)methyl)piperazin-1-yl)-benzoate (2 g). 6M HCl (20 mL) was added drop wise at 0° C. to a solution of Int. 3C58 (2.1 g) in dioxane (15 mL) and stirred for 6 h at 85° C. The mixture was concentrated and triturated in Et₂O to give Int. 3C57 4-(4-((4-(6-aminopyridin-3-yl)-1-methyl-1H-pyrrolo[2,3-b]pyridin-2-yl)methyl)piperazin-1-yl)benzoic acid (1.5 g).

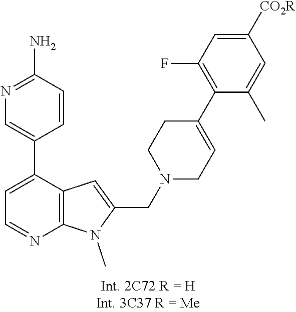

Int. 3C73 (150 mg), N-2-pyridine-5-boronic acid pinacol ester (85 mg), K₂CO₃ (121 mg) and PdDPPFCl₂-DCM (26 mg) were stirred in dioxane/H₂O (4:1, 2.5 mL) ON at 100° C. under an inert atmosphere. The OL (water/EtOAc) was dried, filtered, concentrated and HPLC-purified to give Int. 3C37 methyl 4-(1-((4-(6-aminopyridin-3-yl)-1-methyl-1H-pyrrolo[2,3-b]pyridin-2-yl)methyl)-1,2,3,6-tetrahydropyridin-4-yl)-3-fluoro-5-methylbenzoate (47 mg). LiOH H₂O (9 mg) and Int. 3C37 (10 mg) were stirred ON in THF/H₂O (1:1, 4 mL). Et₃N·HCl (43 mg) was added and the mixture was concentrated to give Int. 2C72 4-(1-((4-(6-aminopyridin-3-yl)-1-methyl-1H-pyrrolo[2,3-b]pyridin-2-yl)methyl)-1,2,3,6-tetrahydropyridin-4-yl)-3-fluoro-5-methylbenzoic acid (10 mg).

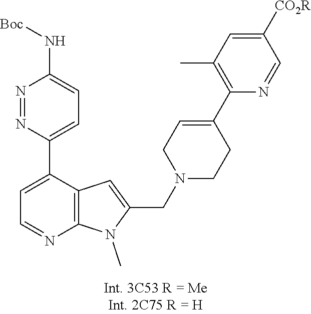

Int. 2C76 (0.30 g), tert-butyl (6-bromopyridazin-3-yl) carbamate (0.10 g), K₂CO₃ (0.30 mg) and PdDPPFCl₂-DCM (20 mg) were stirred in dioxane/water (4:1, 5 mL) ON at 80° C. under an inert atmosphere. The OL (water/EtOAc) was dried, filtered, concentrated, and HPLC-purified to give Int. 3C53 methyl 1′-((4-(6-((tert-butoxycarbonyl)amino)pyridazin-3-yl)-1-methyl-1H-pyrrolo[2,3-b]pyridin-2-yl)methyl)-3-methyl-1′, 2′, 3′, 6′-tetrahydro-[2,4′-bipyridine]-5-carboxylate (0.14 g). Int. 2C75 1′-((4-(6-((tert-butoxycarbonyl)-amino)pyridazin-3-yl)-1-methyl-1H-pyrrolo[2,3-b]pyridin-2-yl)-methyl)-3-methyl-1′, 2′, 3′, 6′-tetrahydro-[2,4′-bipyridine]-5-carboxylic acid (20 mg) was prepared similarly to Int. 2C72 from Int. 3C53 (20 mg).

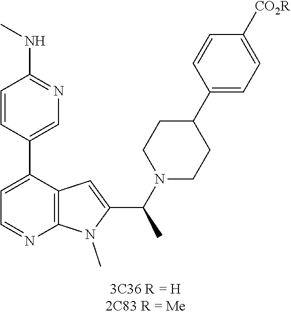

Int. 3E35 (3. 0 g), N-methyl-5-(4,4,5,5-tetramethyl-1,3,2-dioxaborolan-2-yl)pyridin-2-amine (1.8 g), NaHCO₃ (1.1 g), and PdDPPFCl₂-DCM (0.27 g) were stirred 16 h in dioxane/H₂O (4:1, 30 mL) at 85° C. under an inert atmosphere. The OL (water/EtOAc) was dried, filtered, concentrated, and purified by FC (PE/EtOAc 1:1 to 2:3) to give Int. 2C83 methyl(S)-4-(1-(1-(1-methyl-4-(6-(methylamino)-pyridin-3-yl)-1H-pyrrolo[2,3-b]pyridin-2-yl)ethyl)piperidin-4-yl)benzoate (3.0 g). LiOH H₂O (0.89 g) and Int. 2C83 (2.1 g) were stirred 16 h in THF/MeOH/H₂O (2:2:1, 10 mL) at 0° C. to RT. The residue after concentration was stirred in water (pH adjusted aq. citric acid), filtered, and dried to give Int. 3C36 (S)-4-(1-(1-(1-methyl-4-(6-(methylamino)pyridin-3-yl)-1H-pyrrolo[2,3-b]pyridin-2-yl)ethyl)piperidin-4-yl)benzoic acid (1.9 g).

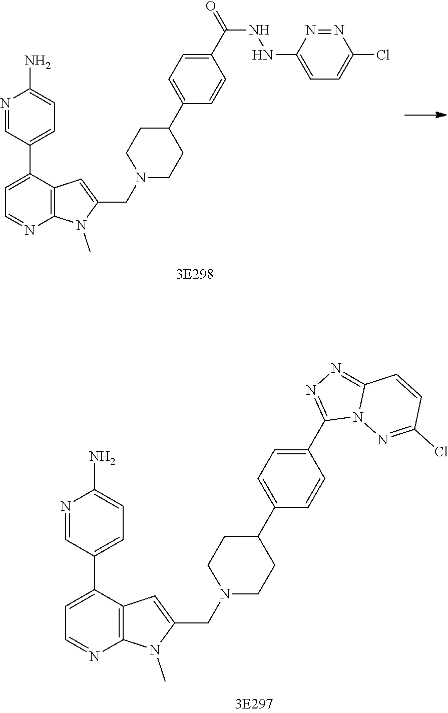 [SMILES: Cn1c(CN2CCC(c3ccc(C(=O)NNc4ccc(Cl)nn4)cc3)CC2)cc2c(-c3ccc(N)nc3)ccnc21 ; Cn1c(CN2CCC(c3ccc(-c4nnc5ccc(Cl)nn45)cc3)CC2)cc2c(-c3ccc(N)nc3)ccnc21]

Int. 3E23 (0.17 g) and HATU (0.2 g) were stirred 4 h in DMF/DIPEA (5.6:1, 1.8 mL). 3-Chloro-6-hydrazineyl-pyridazine (67 mg) was added and stirring continued ON. The OL (2MeTHF/water) was washed with brine, dried, concentrated, and purified by FC (DCM/MeOH/Et₃N 1:0:0 to 90:8.5:0.5) to give Int. 3E298 4-[1-[4-(6-amino-3-pyridyl)-1-methyl-pyrrolo[2,3-b]-pyridin-2-yl]methyl]-4-piperidyl]-N′-(6-chloropyridazin-3-yl)benzohydrazide (0.12 g). This material was stirred 3 h in AcOH (4 mL) at 90° C., concentrated, and purified by FC (DCM/MeOH/Et₃N 95:4.5:0.5) to give Int. 1O1/3E297

5-[2-[[4-[4-(6-chloro-[1,2,4]triazolo[4,3-b]pyridazin-3-yl)phenyl]-1-piperidyl]-methyl]-1-methyl-pyrrolo[2,3-b]pyridin-4-yl]pyridin-2-amine (77 mg).

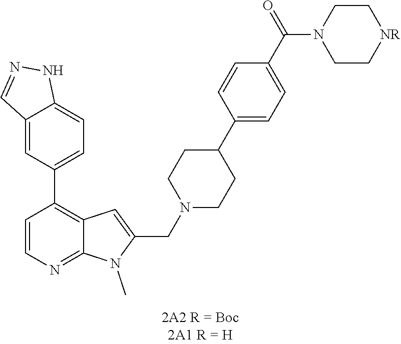

Int. 3A2 (0.20 g, HCl salt), tert-butyl piperazine-1-carboxylate (64 mg), and HATU (0.16 g) were stirred 2 h in DMF/DIPEA (5.7:1, 4 mL). The OL (EtOAc/water) was washed with brine, dried, concentrated, and HPLC-purified to give Int. 2A2 tert-butyl 4-[4-[1-[[1-methyl-4-(1-tetrahydro-pyran-2-ylindazol-5-yl) pyrrolo[2,3-b]-pyridin-2-yl]methyl]-4-piperidyl]benzoyl]-piperazine-1-carboxylate (0.21 g). This material was stirred 1 h in DCM/TFA (2:1, 6 mL). The mixture purified by SCX to give Int. 2A1 (4-(1-((4-(1H-indazol-5-yl)-1-methyl-1H-pyrrolo[2,3-b]pyridin-2-yl)methyl)piperidin-4-yl)phenyl) (piperazin-1-yl) methanone (0.15 g).

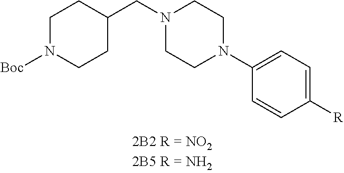

Int. 2B2 tert-butyl 4-((4-(4-nitrophenyl) piperazin-1-yl)methyl)piperidine-1-carboxylate (0.45 g) was prepared from 1-fluoro-4-nitrobenzene similarly to Int. 2I4.

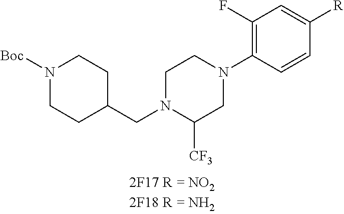

Int. 3E139 (0.36 g), 1,2-difluoro-4-nitrobenzene (162 mg), and K₂CO₃ (0.42 g) were stirred 48 h at 80° C. in ACN (5 mL). The OL (EtOAc/water) was concentrated and purified by FC (hexane/EtOAc 7:3) to give Int. 2F17 tert-butyl 4-((4-(2-fluoro-4-nitrophenyl)-2-(trifluoro-methyl)piperazin-1-yl)methyl)piperidine-1-carboxylate (0.30 g). Int. 2F17 (0.1 g) was hydrogenated ON using 10% Pd/C (20 mg) in THF/dioxane (3:1, 8 mL), filtered, and concentrated to give Int. 2F18 tert-butyl 4-((4-(4-amino-2-fluorophenyl)-2-(trifluoromethyl)piperazin-1-yl)methyl)-piperidine-1-carboxylate (50 mg).

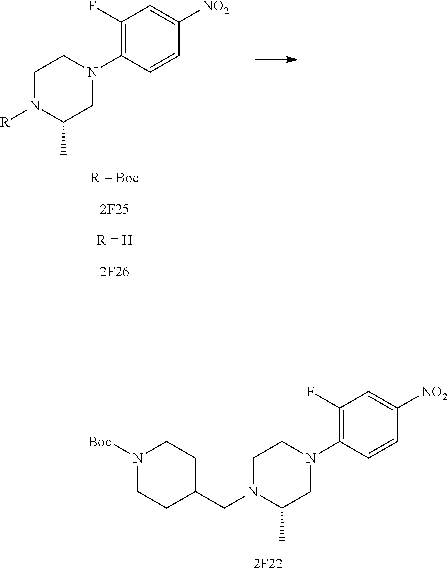

K₂CO₃ (1.36 g), 1,2-difluoro-4-nitro-benzene (0.52 g), and tert-butyl(S)-2-methyl-piperazine-1-carboxylate (0.65 g) were stirred ON in DMF (3 mL) at 70° C. The OL (EtOAc/water) was washed with 3M aq. CaCl₂) and brine, concentrated, and purified by FC (heptane/EtOAc 1:0 to 3:1) to give Int. 2F25 tert-butyl(S)-4-(2-fluoro-4-nitro-phenyl)-2-methylpiperazine-1-carboxylate (0.95 g). This material was stirred 1.5 h in 2.7M HCl in dioxane (15 mL) and concentrated to give Int. 2F26 (S)-1-(2-fluoro-4-nitro-phenyl)-3-methylpiperazine (0.77 g, HCl salt). Int. 2F26 (0.68 g) tert-butyl 4-formyl-piperidine-1-carboxylate (0.96 g) were stirred 0.25 h in DMF/DIPEA (5.3:1, 12 mL). STAB (2.4 g) was added and stirring continued ON. The OL (EtOAc/water) was washed with 3M aq. CaCl₂) and brine, concentrated, and purified by FC (heptane/EtOAc 1:0 to 3:2) to give Int. 2F22 tert-butyl(S)-4-((4-(2-fluoro-4-nitrophenyl)-2-methylpiperazin-1-yl)methyl)-piperidine-1-carboxylate (0.74 g).

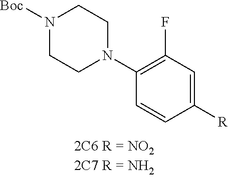

1,2-Difluoro-4-nitrobenzene (0.50 g), tert-butyl piperazine-1-carboxylate (0.70 g), and K₂CO₃ (0.65 g) were stirred ON in DMF (5 mL) at 80° C. The OL (water/EtOAc) was dried, concentrated, and purified by FC (EtOAc/pentane 0:1 to 1:4) to give Int. 2C6 tert-butyl 4-(2-fluoro-4-nitro-phenyl) piperazine-1-carboxylate (0.53 mg). Int. 2C6 (0.5 g), iron powder (0.43 g), and NH₄Cl (0.41 g) were stirred 2 h at 80° C. in THF/EtOH/water (4:4:3, 11 mL). The filtrate after filtration was concentrated and purified by FC (hexane/EtOAc 4:1 to 1:1) to give Int. 2C7 tert-butyl 4-(4-amino-2-fluorophenyl)-piperazine-1-carboxylate (0.40 g).

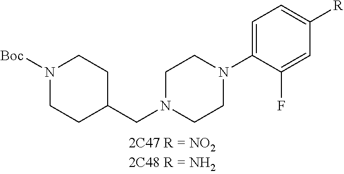

Int. 3E5 (1.07 g), 3,4-difluoronitrobenzene (0.50 g), and K₂CO₃ (0.65 g) were stirred ON in DMF (5 mL) at 80° C. The OL (EtOAc/water) was dried, concentrated, and purified by FC (pentane/EtOAc 1:0 to 1:4) to give Int. 2C47 tert-butyl 4-[[4-(2-fluoro-4-nitro-phenyl)-piperazin-1-yl] methyl]-piperidine-1-carboxylate (0.47 g). Int. 2C47 (0.44 g), NH₄Cl (0.28 g), and iron powder (0.29 g) were stirred 2 h in THF/EtOH/water (4:4:3, 11 mL) at 80° C., filtered, concentrated, and purified by FC (hexane/EtOAc 1:0 to 1:1) to give Int. 2C48 tert-butyl 4-[[4-[4-[(2,6-dioxo-3-piperidyl)-amino]-2-fluoro-phenyl]-piperazin-1-yl]-methyl]piperidine-1-carboxylate (0.30 g).

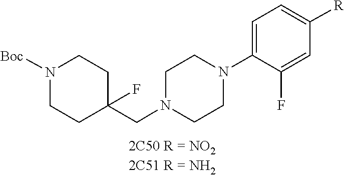

Int. 2C50 (0.63 g) was prepared from 1,2-difluoro-4-nitrobenzene and Int. 3E143 similarly to Int. 2I4. Int. 2C49 3-((3-fluoro-4-(4-((4-fluoropiperidin-4-yl)methyl)-piperazin-1-yl)phenyl)amino)-piperidine-2,6-dione (0.17 g) was prepared from Int. 2C50 similarly to Int. 2N5. Int. 2C51 was prepared from 1,2-difluoro-4-nitrobenzene and Int. 3E143 similarly to Int. 2C48.

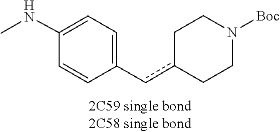

PdDPPFCl₂-DCM (0.22 g), tert-butyl 4-((4,4,5,5-tetramethyl-1,3,2-dioxaborolan-2-yl)methylene) piperidine-1-carboxylate (0.87 g), and 4-bromo-N-methylaniline (0.50 g) were degassed in dioxane/10% aq. Na₂CO₃ (1:1.2, 12.4 mL) and stirred 2 h at 100° C. The OL (EtOAc/brine) was dried, concentrated, and purified by FC (heptane/EtOAc 1:0 to 0:1) to give Int. 2C59 tert-butyl 4-(4-(methylamino)-benzylidene) piperidine-1-carboxylate (0.65 g). Int. 2C59 (0.53 g) was hydrogenated 2 h using 10% Pd/C (0.1 g) in MeOH (8.7 mL), filtered, concentrated, and purified by FC (heptane/EtOAc 1:0 to 0:1) to give Int. 2C58 tert-butyl 4-(4-(methylamino) benzyl) piperidine-1-carboxylate (1.0 g).

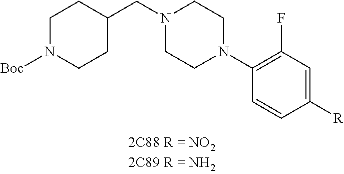

3,4-Difluoro-nitrobenzene (4.0 g), Int. 3E5 (8.6 g), and K₂CO₃ (5.2 g) were stirred ON in DMF (5 mL) at 80° C. The OL (water/EtOAc) was washed with brine, dried, concentrated, and purified by FC (hexane/EtOAc 1:0 to 4:1) to give Ind 2C88 tert-butyl 4-((4-(2-fluoro-4-nitrophenyl) piperazin-1-yl)methyl)piperidine-1-carboxylate (6.4 g). Int. 2C63 (8.0 g), iron powder (5.3 g), and NH₄Cl (5.1 g) were stirred 2 h in THF/EtOH/water (1.6:1.6:1, 104 mL) at 80° C. The mixture was filtered, concentrated, and purified by FC (hexane/EtOAc 4:1 to 1:1) to give Int. 2C89 tert-butyl 4-[[4-(4-amino-2-fluoro-phenyl)-piperazin-1-yl]methyl]piperidine-1-carboxylate (6.0 g).

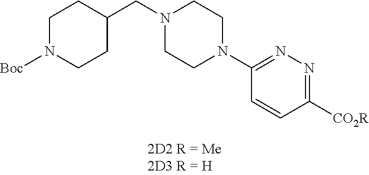

Methyl 6-chloropyridazine-3-carboxylate (0.5 g) was stirred 0.25 h in DMSO/DIPEA (2:1, 4.5 mL). Int. 3E5 (821 g) was added and stirring continued ON at 120° C. The OL (EtOAc/water) was concentrated and purified by FC (pentane/EtOAc 4:1) to give Int. 2D2 methyl 6-(4-((1-(tert-butoxy-carbonyl) piperidin-4-yl)-methyl)-piperazin-1-yl) pyridazine-3-carboxylate (0.7 g). Int. 2D2 (0.8 g) and LiOH—H₂O (0.4 g) were stirred 6 h in THF/MeOH/water (2:2:1, 8 mL) and concentrated. The OL (DCM/MeOH (9:1)/water) was concentrated and purified by FC (hexane/EtOAc 1:1) to give Int. 2D3 6-(4-((1-(tert-butoxycarbonyl)-piperidin-4-yl)-methyl)-piperazin-1-yl)-pyridazine-3-carboxylic acid (0.65 g).

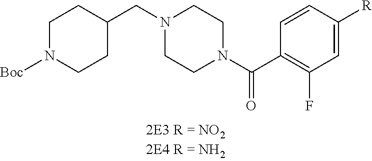

2-Fluoro-4-nitro-benzoic acid (0.16 g), Int. 3E5 (0.20 g), and HATU (322 mg) were stirred ON in DMF/DIPEA (6:1, 3.6 mL). The OL (EtOAc/water) was concentrated. The residue was purified by FC (EtOAc/MeOH 1:0 to 95:5) to give Int. 2E3 tert-butyl 4-((4-(2-fluoro-4-nitrobenzoyl) piperazin-1-yl)methyl)piperidine-1-carboxylate (0.19 g). This material and iron powder (0.2 g) were stirred 0.75 h in EtOH/water (1:1, 3 mL) and AcOH (0.05 mL) at 80° C. and filtered and concentrated. The OL (DCM/water) was washed with sat. aq. NaHCO₃ and brine, concentrated, and purified by SCX give Int. 2E4 tert-butyl 4-((4-(4-amino-2-fluorobenzoyl)-piperazin-1-yl)-methyl)piperidine-1-carboxylate (0.18 g).

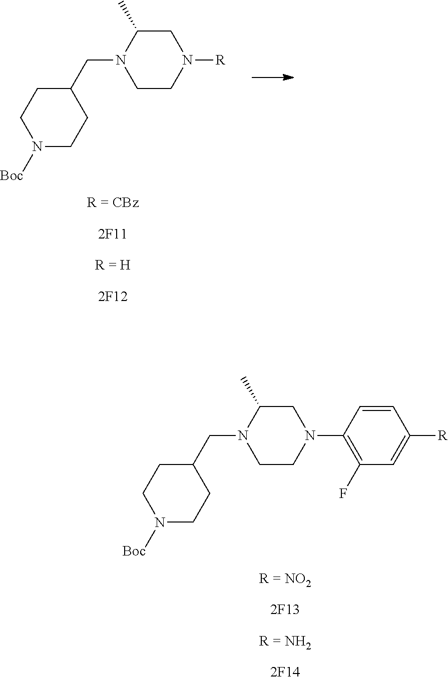

Benzyl (R)-3-methylpiperazine-1-carboxylate (12 g, HCl salt) and tert-butyl 4-formyl-piperidine-1-carbo-xylate (11 g) were stirred 2 h in DCE/DIPEA (5:1, 240 mL) at 0° C. to RT. STAB (19 g) was added and stirring continued ON before concentration. The OL (water/DCM) was concentrated and purified by FC (hexane/EtOAc 4:1 to 3:2) to give Int. 2F11 benzyl (R)-4-((1-(tert-butoxycarbonyl) piperidin-4-yl)methyl)-3-methyl-piperazine-1-carboxylate (12 g). Int. 2F11 (5 g) and 10% Pd/C (1.5 g) were stirred ON in EtOAc/THF (2:1, 75 mL) under a H₂ atmosphere (70 psi). The filtrate after concentration was dried to give Int. 2F12 tert-butyl (R)-4-((2-methylpiperazin-1-yl)-methyl)-piperidine-1-carboxylate (3.1 g). Int. 2F12 (9.0 g), K₂CO₃ (12 g) and 1,2-difluoro-4-nitrobenzene (6 g) were stirred 4 h in DMF (50 mL) at 0-80° C. and filtered. The OL (EtOAc/water) was concentrated and purified by FC (pentane/EtOAc 7:3 to 3:2) to give Int. 2F13 tert-butyl (R)-4-((4-(2-fluoro-4-nitrophenyl)-2-methyl-piperazin-1-yl)methyl)-piperidine-1-carboxylate (4.5 g). Int. 2F13 (4.2 g), NH₄Cl (2.5 g), and iron powder (2.7 g) were stirred 6 h in THF/EtOH/water (2:2:1, 62 mL) at 80° C. and concentrated. The OL (water/DCM) was concentrated and purified by FC (hexane/EtOAc 3:1 to 3:2) to give Int. 2F14 tert-butyl (R)-4-((4-(4-amino-2-fluoro-phenyl)-2-methylpiperazin-1-yl)methyl)-piperidine-1-carboxylate (3.8 g).

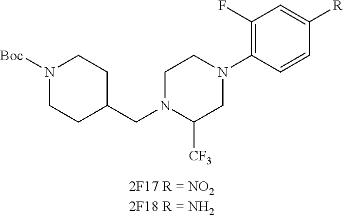

Int. 3E139 (0.36 g), 1,2-difluoro-4-nitrobenzene (162 mg), and K₂CO₃ (0.42 g) were stirred 48 h at 80° C. in ACN (5 mL). The OL (EtOAc/water) was concentrated and purified by FC (hexane/EtOAc 7:3) to give Int. 2F17 tert-butyl 4-((4-(2-fluoro-4-nitrophenyl)-2-(trifluoro-methyl)piperazin-1-yl)methyl)piperidine-1-carboxylate (0.30 g). Int. 2F17 (0.1 g) was hydrogenated ON using 10% Pd/C (20 mg) in THF/dioxane (3:1, 8 mL), filtered, and concentrated to give Int. 2F18 tert-butyl 4-((4-(4-amino-2-fluorophenyl)-2-(trifluoromethyl)piperazin-1-yl)methyl)-piperidine-1-carboxylate (50 mg).

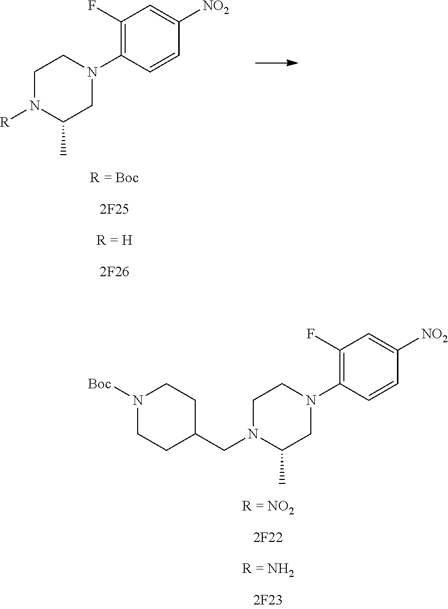

K₂CO₃ (1.36 g), 1,2-difluoro-4-nitro-benzene (0.52 g), and tert-butyl(S)-2-methyl-piperazine-1-carboxylate (0.65 g) were stirred ON in DMF (3 mL) at 70° C. The OL (EtOAc/water) was washed with 3M aq. CaCl₂) and brine, concentrated, and purified by FC (heptane/EtOAc 1:0 to 3:1) to give Int. 2F25 tert-butyl(S)-4-(2-fluoro-4-nitro-phenyl)-2-methylpiperazine-1-carboxylate (0.95 g). This material was stirred 1.5 h in 2.7M HCl in dioxane (15 mL) and concentrated to give Int. 2F26 (S)-1-(2-fluoro-4-nitro-phenyl)-3-methylpiperazine (0.77 g, HCl salt). Int. 2F26 (0.68 g) tert-butyl 4-formylpiperidine-1-carboxylate (0.96 g) were stirred 0.25 h in DMF/DIPEA (5.3:1, 12 mL). STAB (2.4 g) was added and stirring continued ON. The OL (EtOAc/water) was washed with 3M aq. CaCl₂) and brine, concentrated, and purified by FC (heptane/EtOAc 1:0 to 3:2) to give Int. 2F22 tert-butyl(S)-4-((4-(2-fluoro-4-nitrophenyl)-2-methyl-piperazin-1-yl)methyl)-piperidine-1-carboxylate (0.74 g).

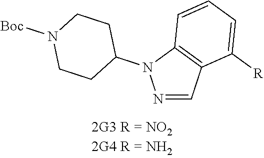

tert-Butyl 4-((methylsulfonyl)oxy) piperidine-1-carboxylate (0.38 g) was added over 5 h to a mixture of 4-nitro-1H-indazole (0.20 g) and K₂CO₃ (0.34 g) in DMF (6 mL) at 100° C. and stirring was continued ON. The OL (EtOAc/water) was concentrated and purified by FC (heptane/EtOAc 1:0 to 0:1) to give Int. 2G3 tert-butyl 4-(4-nitro-indazol-1-yl)-piperidine-1-carboxylate (0.42 g). Int. 2G3 (0.42 g) was hydrogenated 4 h using 10% Pd/C (50 mg) in EtOH (4 mL), filtered, concentrated, and HPLC-purified to give Int. 2G4 tert-butyl 4-(4-aminoindazol-1-yl)-piperidine-1-carboxylat (63 mg).

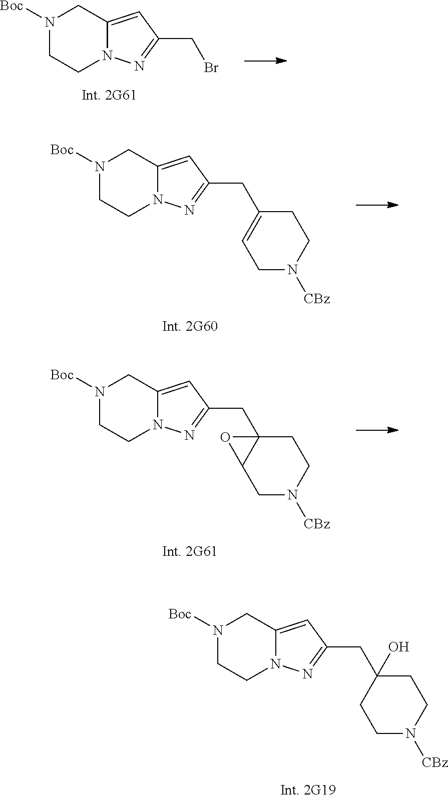

CBr₄ (15.7 g), PPh₃ (15.5 g), and tert-butyl 2-(hydroxymethyl)-6,7-dihydropyrazolo[1,5-a]pyrazine-5(4H)-carboxylate (10.0 g) were stirred 16 h in DCM (80 mL). The OL (water/DCM) was dried, filtered, concentrated and purified by FC (hexane/EtOAc 2:1) to give Int. 2G61 tert-butyl 2-(bromomethyl)-6,7-dihydropyrazolo [1,5-a]pyrazine-5(4H)-carboxylate (10.0 g). Int. 2G61 (10.0 g), 1-phenyl-2-(4-(4,4,5,5-tetramethyl-1,3,2-dioxaborolan-2-yl)-3,6-dihydropyridin-1(2H)-yl)-212-ethan-1-one (10.9 g), K₂CO₃ (13.1 g) and PdDPPFCl₂-DCM (2.6 g) were stirred 16 h in dioxane/H₂O (7:3, 100 mL) under an inert atmosphere. The mixture was filtered. The OL (water/EtOAc) was dried, filtered, concentrated, and purified by FC (hexane/EtOAc 2:1) to give Int. 2G60 tert-butyl 2-((1-((benzyloxy) carbonyl)-1,2,3,6-tetrahydropyridin-4-yl)methyl)-6,7-dihydropyrazolo [1,5-a]pyrazine-5(4H)-carboxylate (7.0 g). m-CPBA (23 g) and Int. 2G60 (30 g) were stirred 4 h in DCM (200 mL) at 0° C. to RT. The OL (aq. NaHCO₃/DCM) was dried, filtered and concentrated to give Int. 2G59 benzyl 6-((5-(tert-butoxycarbonyl)-4,5,6,7-tetrahydropyrazolo [1,5-a]pyrazin-2-yl)methyl)-7-oxa-3-azabicyclo [4.1.0] heptane-3-carboxylate (28 g). Int. 2G59 (17 g) and Pd/C 10% wt. (7 g) were hydrogenated at 60 psi in dioxane (170 mL), filtered, concentrated and HPLC-purified to give Int. 2G19 tert-butyl 2-((4-hydroxypiperidin-4-yl)methyl)-6,7-dihydropyrazolo [1,5-a]pyrazine-5(4H)-carboxylate (11 g).

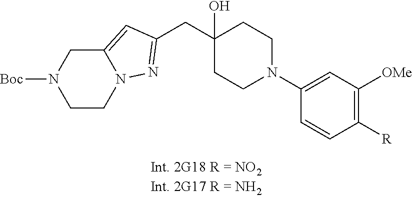

K₂CO₃ (7.3 g), Int. 2G19 (6.5 g), and 4-fluoro-2-methoxy-1-nitrobenzene (3 g) were stirred 15 h in DMF (30 mL) at 70° C. The OL (water/DCM/MeOH) was dried, filtered, concentrated, and purified by FC (EtOAc) to give Int. 2G18 tert-butyl 2-((4-hydroxy-1-(3-methoxy-4-nitrophenyl) piperidin-4-yl)methyl)-6,7-dihydropyrazolo [1,5-a]pyrazine-5(4H)-carboxylate (3.1 g). Int. 2G18 (2.5 g) was hydrogenated for 16 h using 10% Pd/C (1.2 g) in MeOH (30 mL), filtered, and concentrated to give Int. 2G17 tert-butyl 2-((1-(4-amino-3-methoxyphenyl)-4-hydroxy-piperidin-4-yl)methyl)-6,7-dihydropyrazolo[1,5-a]-pyrazine-5(4H)-carboxylate (1.7 g).

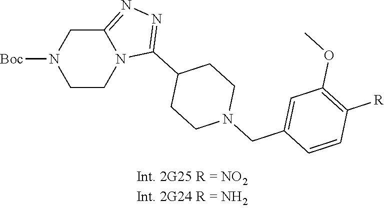

3-Methoxy-4-nitrobenzaldehyde (0.32 g), tert-butyl 3-(piperidin-4-yl)-5,6-dihydro-[1,2,4]triazolo [4,3-a]pyrazine-7 (8H)-carboxylate (0.50 g), and NaCNBH₃ (0.31 g) were stirred ON in DCE/Et₃N (15:1, 10.7 mL). The mixture was filtered, concentrated, and purified by FC (MTBE/MeOH) to give Int. 2G25 tert-butyl 3-(1-(3-methoxy-4-nitrobenzyl) piperidin-4-yl)-5,6-dihydro-[1,2,4]triazolo [4,3-a]pyrazine-7 (8H)-carboxylate (0.30 g). Int. 2G25 (0.30 g) was hydrogenated ON using Pt/C 10% wt. (50 mg) in MeOH (10 mL), filtered, and concentrated to give Int. 2G24 tert-butyl 3-(1-(4-amino-3-methoxybenzyl)-piperidin-4-yl)-5,6-dihydro-[1,2,4]triazolo [4,3-a]pyrazine-7 (8H)-carboxylate (0.15 g).

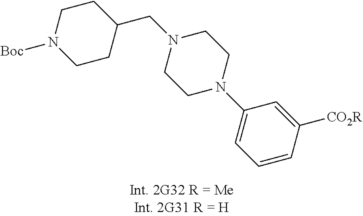

Methyl 3-bromobenzoate (3 g), Cs₂CO₃ (9.1 g), BINAP (1.7 g), Pd (OAc) 2 (0.31 g), and tert-butyl 4-(piperazin-1-ylmethyl)piperidine-1-carboxylate were stirred 16 h in dioxane (20 mL) at 100° C. under an inert atmosphere. The mixture was filtered, concentrated, and purified by FC (hexane/EtOAc 3:2) to give Int. 2G32 tert-butyl 4-((4-(3-(methoxycarbonyl)phenyl) piperazin-1-yl)methyl)piperidine-1-carboxylate (3.3 g). LiOH H₂O (2.4 g) and Int. 2G32 (4 g) were stirred 16 h in THF/MeOH/H₂O (2:1:1, 40 mL) at 0° C. to RT. The mixture was concentrated. The OL (aq. 10% citric acid/EtOAc) was concentrated to give Int. 2G31 3-(4-((1-(tert-butoxycarbonyl) piperidin-4-yl)-methyl)-piperazin-1-yl)benzoic acid (3.3 g).

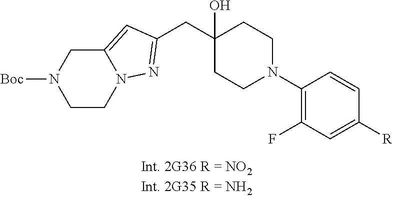

K₂CO₃ (0.22 g), Int. 2G19 (0.20 g), and 1,2-difluoro-4-nitrobenzene (91 mg) were stirred ON in ACN (2 mL) at 80° C. The mixture was filtered, concentrated, and HPLC-purified to give Int. 2G36 tert-butyl 2-((1-(2-fluoro-4-nitrophenyl)-4-hydroxypiperidin-4-yl)methyl)-6,7-dihydropyrazolo-[1,5-a]pyrazine-5(4H)-carboxylate (69 mg). Int. 2G36 (69 mg) and Pd/C (50 mg) were hydrogenated ON in MeOH (5 mL), filtered, and concentrated to give Int. 2G35 tert-butyl 2-((1-(4-amino-2-fluoro-phenyl)-4-hydroxypiperidin-4-yl)methyl)-6,7-dihydropyrazolo [1,5-a]pyrazine-5(4H)-carboxylate (65 mg).

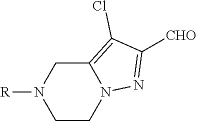

NCS (0.53 g) and tert-butyl 2-formyl-6,7-dihydropyrazolo [1,5-a]pyrazine-5(4H)-carboxylate (0.50 g) were stirred 16 h in DMF (10 mL). The OL (brine/EtOAc) was dried, filtered and concentrated to give Int. 2G39 tert-butyl 3-chloro-2-formyl-6,7-dihydropyrazolo [1,5-a]pyrazine-5(4H)-carboxylate (0.54 g).

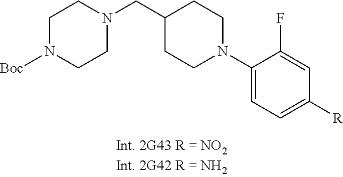

1,2-Difluoro-4-nitrobenzene (1.1 g), tert-butyl 4-(piperidin-4-ylmethyl)piperazine-1-carboxylate (2.0 g), and K₂CO₃ (2.9 g) were stirred ON in DMF (50 mL) at 100° C. under an inert atmosphere. The OL (water/EtOAc) dried, filtered, and concentrated to give Int. 2G43 tert-butyl 4-((1-(2-fluoro-4-nitro-phenyl) piperidin-4-yl)methyl)piperazine-1-carboxylate (2.2 g). Int. 2G43 (2.2 g) was hydrogenated ON using Pd/C (55 mg) in MeOH (50 mL), filtered, and concentrated to give Int. 2G42 tert-butyl 4-((1-(4-amino-2-fluorophenyl) piperidin-4-yl)methyl)piperazine-1-carboxylate (1.5 g).

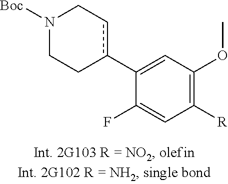

1-Bromo-2-fluoro-5-methoxy-4-nitrobenzene (5 g), N-Boc-DHP-4-boronic acid pinacol ester (6.5 g) and K₂CO₃ (9 g) were mixed in dioxane/H₂O (40:1, 10 mL). PdDPPFCl₂-DCM (1.5 g) was added and the mixture stirred for 12 h at 80° C. under an atmosphere of argon. The mixture was filtered and concentrated to give crude Int. 2G103 tert-butyl 4-(2-fluoro-5-methoxy-4-nitrophenyl)-3,6-dihydropyridine-1(2H)-carboxylate (7.4 g). Int. 2G103 (7.2 g) was hydrogenated for 12 h using Pd/C (0.5 g) in MeOH (100 mL), filtered, and concentrated to give Int. 2G102 tert-butyl 4-(4-amino-2-fluoro-5-methoxyphenyl) piperidine-1-carboxylate (6.0 g).

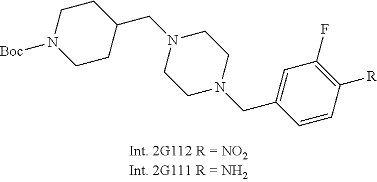

STAB (10 g), 3-fluoro-4-nitrobenzaldehyde (2 g), and tert-butyl 4-(piperazin-1-ylmethyl)piperidine-1-carboxylate (3.3 g) were stirred 12 h in DCE (60 mL). The OL (water/DCE) was concentrated to give Int. 2G112 tert-butyl 4-((4-(3-fluoro-4-nitrobenzyl) piperazin-1-yl)methyl)-piperidine-1-carboxylate (1.9 g). Int. 2G112 (1.9 g) and Pd/C (0.30 g) were hydrogenated in MeOH (30 mL), filtered, and concentrated to give Int. 2G111 tert-butyl 4-((4-(4-amino-3-fluorobenzyl)-piperazin-1-yl)methyl)-piperidine-1-carboxylate (1.5 g).

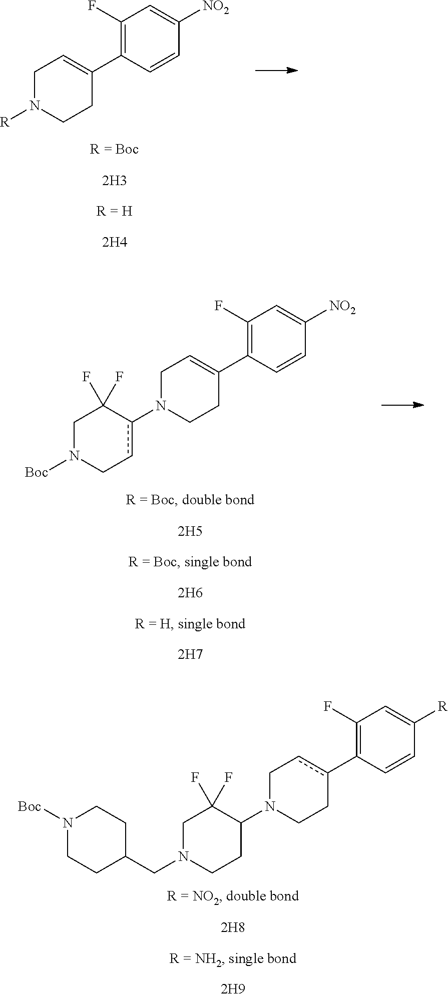

1-Bromo-2-fluoro-4-nitrobenzene (6 g), PdCl₂ (PPh₃) 2 (1.0 g), and tert-butyl 4-(4,4,5,5-tetramethyl-1,3,2-dioxa-borolan-2-yl)-3,6-dihydropyridine-1(2H)-carboxylate (10 g) were degassed in dioxane/water (12:1, 65 mL) and stirred ON at 90° C. The OL (EtOAc/water) was concentrated and purified by FC (pentane/EtOAc 7:3 to 65:35) to give Int. 2H3 tert-butyl 4-(2-fluoro-4-nitrophenyl)-3,6-dihydro-pyridine-1(2H)-carboxylate (8 g). This material was stirred 2 h in 2M HCl in 1,4-dioxane (160 mL) at 0° C. to RT, concentrated, and triturated in pentane to give Int. 2H4 4-(2-fluoro-4-nitro-phenyl)-1,2,3,6-tetrahydropyridine (5 g, HCl salt). Int. 2H4 (6.0 g) and NaOAc (4.8 g) were stirred 0.5 h in ACN/toluene (1:2, 150 mL). tert-Butyl 3,3-difluoro-4-oxo-piperidine-1-carboxylate (8.7 g) and AcOH (5 mL) were added and stirring continued ON at 100° C. The filtrate after filtration was concentrated to give Int. 2H5 tert-butyl 3′, 3′-difluoro-4-(2-fluoro-4-nitro-phenyl)-3,3′, 6,6′-tetrahydro-2H-[1,4′-bipyridine]-1′ (2′H)-carboxylate (7 g). This material and NaCNBH₃ (5.0 g) were stirred ON in MeOH/DCE/AcOH (6.7:6.7:1, 43 mL). The residue after concentration was diluted with EtOAc and filtered. The residue after concentration of the filtrate was purified by FC (pentane/EtOAc 1:0 to 7:3) to give Int. 2H6 tert-butyl 3,3-difluoro-4-(4-(2-fluoro-4-nitro-phenyl)-3,6-dihydropyridin-1(2H)-yl) piperidine-1-carboxylate (3 g). Int. 2H6 (1.5 g) was stirred 2 h in 2M HCl in dioxane (20 mL) at 0° C. to RT. The mixture was concentrated to give Int. 2H7 1-(3,3-difluoro-piperidin-4-yl)-4-(2-fluoro-4-nitrophenyl)-1,2,3,6-tetrahydropyridine (1.2 g, HCl salt). This material and tert-butyl 4-formyl-piperidine-1-carboxylate (0.81 g) were stirred 4 h in DCE/DIPEA (10:1, 17 mL). STAB (1.4 g) was added and stirring was continued ON at 0° C. to RT. The OL (water/DCM) was concentrated and purified by FC (pentane/EtOAc 1:1 to 2:3) to give Int. 2H8 tert-butyl 4-((3,3-difluoro-4-(4-(2-fluoro-4-nitro-phenyl)-3,6-dihydropyridin-1(2H)-yl) piperidin-1-yl)methyl)-piperidine-1-carboxylate (1.1 g). This material was hydrogenated ON using 50% Pd/C (0.5 g) in EtOAc (6 mL), filtered, and concentrated to give Int. 2H9 tert-butyl 4-((4-(4-amino-2-fluoro-phenyl)-3′, 3′-difluoro-[1,4′-bipiperidin]-1′-yl)-methyl)piperidine-1-carboxylate (1.0 g).

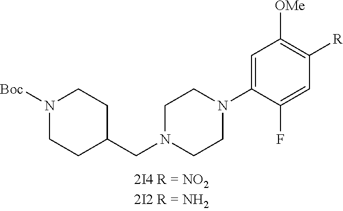

1,2-Difluoro-4-methoxy-5-nitro-benzene (2.7 g), tert-butyl 4-(piperazin-1-ylmethyl)-piperidine-1-carboxylate (4.9 g), and K₂CO₃ (2.9 g) were stirred ON in DMF (10 mL) at 80° C. The OL (EtOAc/water) was washed with brine, dried, concentrated, and purified by FC (pentane/EtOAc 4:1 to 7:3) to give Int. 2I4 tert-butyl 4-((4-(2-fluoro-5-methoxy-4-nitrophenyl) piperazin-1-yl)methyl)piperidine-1-carboxylate (4 g).

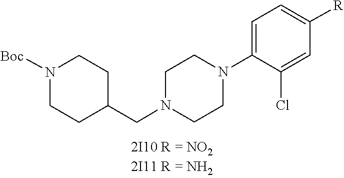

Int. 2I10 tert-butyl 4-((4-(2-fluoro-5-methoxy-4-nitrophenyl) piperazin-1-yl)methyl)-piperidine-1-carboxylate (1.2 g) prepared similarly to Int. 2I4 from 2-chloro-1-fluoro-4-nitrobenzene (1.8 g).

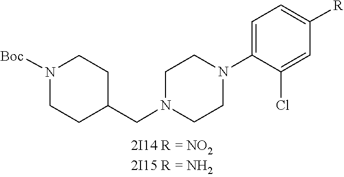

Int. 2I14 tert-butyl 4-((4-(2-fluoro-5-methoxy-4-nitrophenyl) piperazin-1-yl)methyl)piperidine-1-carboxylate (2 g) prepared similarly to Int. 2I4 from 2-fluoro-5-nitrobenzonitrile (1.0 g).

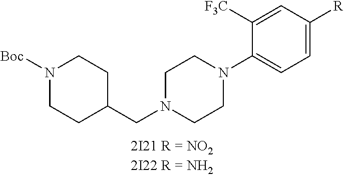

Int. 2I21 tert-butyl 4-((4-(4-nitro-2-(trifluoromethyl)phenyl) piperazin-1-yl)methyl)-piperidine-1-carboxylate (4 g) was prepared from 1-fluoro-4-nitro-2-(trifluoromethyl)-benzene (2.5 g) similarly to Int. 2I4.

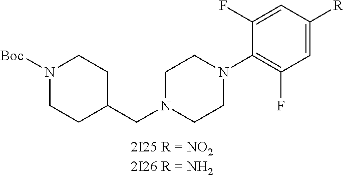

Int. 2I25 tert-butyl 4-((4-(2,6-difluoro-4-nitrophenyl) piperazin-1-yl)methyl)piperidine-1-carboxylate (0.45 g) prepared similarly to Int. 2I4 from 1,2,3-trifluoro-5-nitro-benzene.

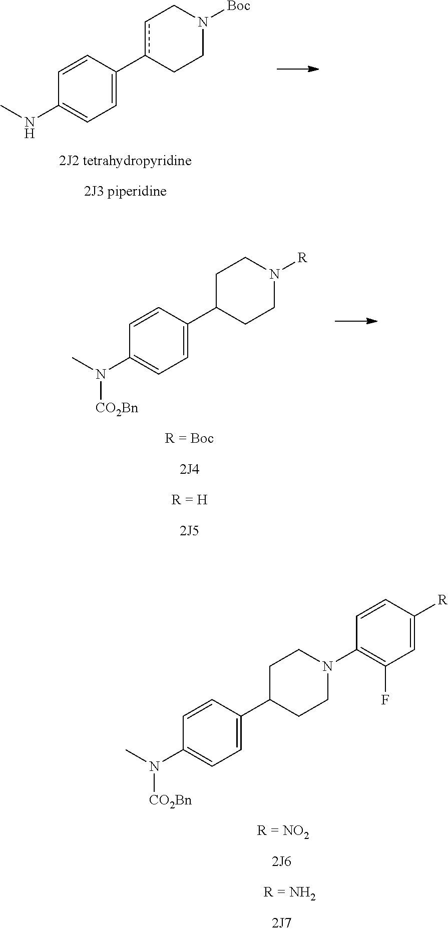

4-Bromo-N-methylaniline (2.4 g), Cs₂CO₃ (13 g), tert-butyl 4-(4,4,5,5-tetramethyl-1,3,2-dioxaborolan-2-yl)-3,6-dihydro-pyridine-1(2H)-carboxylate (4.8 g), and PdDPPFCl₂-DCM (0.5 g) were degassed in dioxane/water (10:1, 44 mL) and stirred ON at 110° C., filtered, concentration, and purified by FC (heptane/EtOAc 1:0 to 4:1) to give Int. 2J2 tert-butyl 4-(4-(methylamino)phenyl)-3,6-dihydropyridine-1(2H)-carboxylate (2.7 g). This material was hydrogenated ON using 10% Pd/C (0.5 g) in MeOH (37 mL), filtered, and concentrated to give Int. 2J3 tert-butyl 4-(4-(methyl-amino)phenyl)-piperidine-1-carboxylate (2.6 g). Int. 2J3 (0.83 g) was stirred in DCM/DIPEA (4:1, 7.5 mL) at 0° C. CB_{z}—C1 (0.55 g) was added and stirring was continued ON at 0° C. to RT. The OL (DCM/water) was washed with brine, concentrated, and purified by FC (hexane/EtOAc 1:0 to 3:2) to give Int. 2J4 tert-butyl 4-(4-(((benzyloxy)-carbonyl) (methyl)-amino)phenyl)-piperidine-1-carboxylate (1.1 g). This material was stirred 1 h in 2.7M HCl in dioxane (15 mL) and concentrated to give Int. 2J5 benzyl methyl (4-(piperidin-4-yl)phenyl)-carbamate (0.79 g, HCl salt). Int. 2J5 (0.25 g), K₂CO₃ (0.29 g), and 1,2-difluoro-4-nitrobenzene (0.11 g) were stirred ON at 70° C. and 48 h at RT. The OL (EtOAc/water) was washed with brine, concentrated, and HPLC-purified to give Int. 2J6 benzyl (4-(1-(2-fluoro-4-nitrophenyl)-piperidin-4-yl)phenyl) (methyl)-carbamate (0.27 g). This material and iron powder (0.33 g) were stirred ON in THF/AcOH (2:1, 9 mL). Iron powder (0.33 g) was added and stirring continued 4 h. The filtrate was SCX-purified to give Int. 2J7 benzyl (4-(1-(4-amino-2-fluoro-phenyl)-piperidin-4-yl)phenyl)-(methyl)-carbamate (0.25 g).

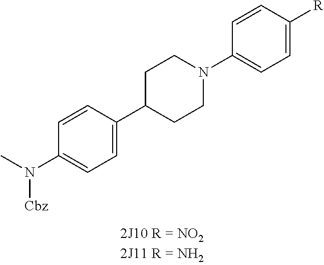

Int. 2J11 was prepared similarly to Int. 2J7 from 1-fluoro-4-nitrobenzene.

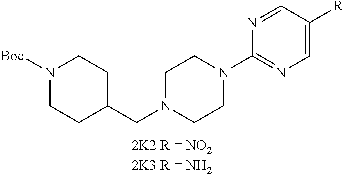

Int. 2K2 tert-butyl 4-((4-(5-nitropyrimidin-2-yl) piperazin-1-yl)methyl)piperidine-1-carboxylate (0.2 g) was prepared from 2-chloro-5-nitropyrimidine (0.10 g) similarly to Int. 2I4.

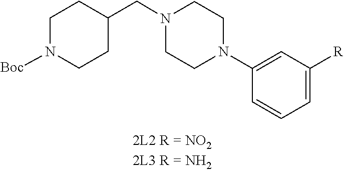

1-Bromo-3-nitrobenzene (0.30 g), tert-butyl 4-(piperazin-1-ylmethyl)piperidine-1-carboxylate (0.51 g), NaOtBu (214 mg), XantPhos (86 mg), and Pd₂dba₃ (136 mg) were degassed in toluene (12 mL) and stirred 48 h at 130° C. Filtration, concentration, and HPLC-purification gave Int. 2L2 tert-butyl 4-((4-(3-nitro-phenyl)-piperazin-1-yl)-methyl)piperidine-1-carboxylate (0.24 g). This material was hydrogenated ON using 10% Pd/C (0.1 g) in EtOH (10 mL), filtered, and concentrated to give Int. 2L3 tert-butyl 4-((4-(3-amino-phenyl)-piperazin-1-yl)methyl)-piperidine-1-carboxylate (0.20 g).

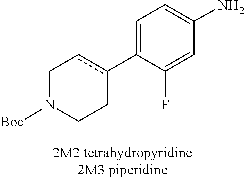

4-Bromo-3-fluoro-aniline (0.22 g), tert-butyl 4-(4,4,5,5-tetramethyl-1,3,2-dioxaborolan-2-yl)-3,6-dihydro-2H-pyridine-1-carboxylate (0.43 g), K₂CO₃ (0.48 g), and PdPDDFCl₂-DCM (42 mg) were degassed in dioxane/water (6:1, 7 mL) and stirred ON at 90° C., filtered, concentrated and purified by FC (heptane/EtOAc 1:0 to 65:35) to give Int. 2M2 tert-butyl 4-(4-amino-2-fluorophenyl)-3,6-dihydropyridine-1(2H)-carboxylate (0.28 g). This material was hydrogenated ON using 10% Pd/C (50 mg) in EtOAc (6 mL), filtered, and concentrated to give Int. 2M3 tert-butyl 4-(4-amino-2-fluorophenyl)-piperidine-1-carboxylate (0.27 g).

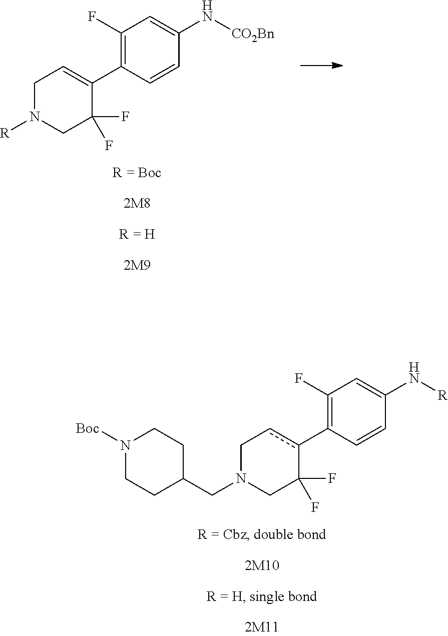

Na₂CO₃ (0.96 g), benzyl (3-fluoro-4-(4,4,5,5-tetramethyl-1,3,2-dioxaborolan-2-yl)phenyl) carbamate (1.4 g), Int. 3E284 (1.7 g), and PdPDDFCl₂-DCM (0.28 g) were degassed in dioxane/water (5:1, 24 mL) and stirred ON at 60° C. The OL (EtOAc/water) was washed with brine, dried, concentrated, and purified by FC (hexane/EtOAc 4:1 to 7:3) to give Int. 2M8 tert-butyl 4-(4-(((benzyloxy) carbonyl)amino)-2-fluorophenyl)-3,3-difluoro-3,6-dihydro-pyridine-1(2H)-carboxylate (1.5 g). This material (1.1 g) was stirred ON in DCM/4M HCl in dioxane (1:1, 22 mL) at 0° C. to RT, concentrated, and triturated in Et₂O/pentane to give Int. 2M9 benzyl (4-(3,3-difluoro-1,2,3,6-tetrahydropyridin-4-yl)-3-fluoro-phenyl)-carbamate (0.76 g, HCl salt).

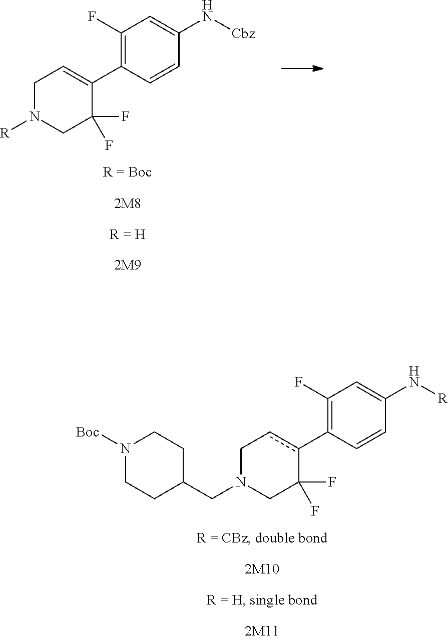

Na₂CO₃ (0.96 g), benzyl (3-fluoro-4-(4,4,5,5-tetramethyl-1,3,2-dioxaborolan-2-yl)-phenyl) carbamate (1.4 g), Int. 3E284 (1.7 g), and PdPDDFCl₂-DCM (0.28 g) were degassed in dioxane/water (5:1, 24 mL) and stirred ON at 60° C. The OL (EtOAc/water) was washed with brine, dried, concentrated, and purified by FC (hexane/EtOAc 4:1 to 7:3) to give Int. 2M8 tert-butyl 4-(4-(((benzyloxy) carbonyl)amino)-2-fluorophenyl)-3,3-difluoro-3,6-dihydro-pyridine-1(2H)-carboxylate (1.5 g). This material (1.1 g) was stirred ON in DCM/4M HCl in dioxane (1:1, 22 mL) at 0° C. to RT, concentrated, and triturated in Et₂O/pentane to give Int. 2M9 benzyl (4-(3,3-difluoro-1,2,3,6-tetrahydro-pyridin-4-yl)-3-fluoro-phenyl)-carbamate (0.76 g, HCl salt). This material (0.73 g) and tert-butyl 4-formylpiperidine-1-carboxylate (0.39 g) were stirred 3 h in DCE/DIPEA (10:1, 19.6 mL) at 0° C. to RT.

STAB (0.78 g) was added and stirring continued ON before concentration. The OL (water/MeOH/DCM (1:9)) was washed with brine, dried, concentrated, and purified by FC (hexane/EtOAc 3:2 to 1:1) to give Int. 2M10 tert-butyl 4-((4-(4-(((benzyloxy)-carbonyl)amino)-2-fluorophenyl)-3,3-difluoro-3,6-dihydropyridin-1(2H)-yl)methyl)-piperidine-1-carboxylate (0.70 g). This material was hydrogenated 48 h using 10% Pd/C (0.25 g) in EtOAc (15 mL), filtered, concentrated, and purified by FC (hexane/EtAOC 1:4 to 0:1) to give Int. 2M11 tert-butyl 4-((4-(4-amino-2-fluorophenyl)-3,3-difluoropiperidin-1-yl)methyl)piperidine-1-carboxylate (0.21 g).

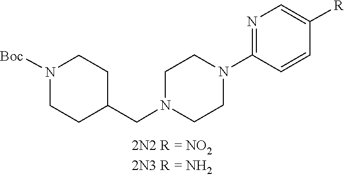

Int. 2N2 tert-butyl 4-((4-(5-nitropyridin-2-yl) piperazin-1-yl)methyl)piperidine-1-carboxylate (0.76 g) was prepared from 2-chloro-5-nitropyridine (0.90 g) similarly to Int. 2I4.

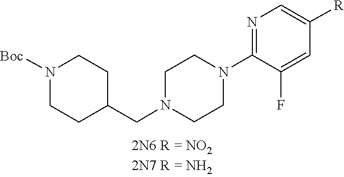

Int. 2N6 tert-butyl 4-((4-(3-fluoro-5-nitropyridin-2-yl) piperazin-1-yl)methyl)piperidine-1-carboxylate (4.5 g) was prepared from 2-chloro-3-fluoro-5-nitropyridine (2.5 g) similarly to Int. 2I4.

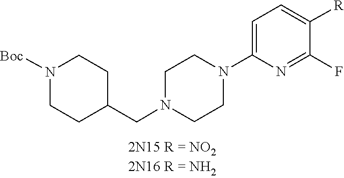

Int. 2N15 tert-butyl 4-((4-(6-fluoro-5-nitropyridin-2-yl) piperazin-1-yl)methyl)piperidine-1-carboxylate (1.0 g) was prepared from 2,6-difluoro-3-nitropyridine similarly to Int. 2I4 using DIPEA instead of K₂CO₃.

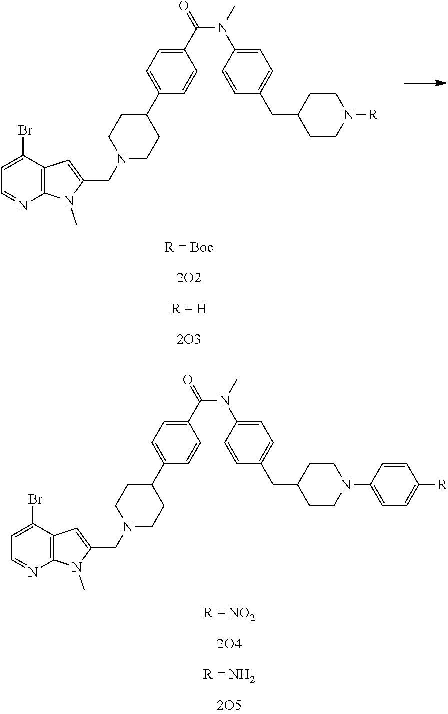

Int. 2O2 tert-butyl 4-(4-(4-(1-((4-bromo-1-methyl-1H-pyrrolo[2,3-b]pyridin-2-yl)methyl)-piperidin-4-yl)-N-methylbenzamido) benzyl) piperidine-1-carboxylate (357 mg) was prepared similarly to Int. 2C18 from Ints. 1AC4/2C58 (0.21 g/0.18 g). This material was stirred 0.5 h in DCM/TFA (4:1, 2.5 mL), concentrated, and purified by SCX to give Int. 2O3 4-(1-((4-bromo-1-methyl-1H-pyrrolo [2,3-b] pyridin-2-yl)-methyl)piperidin-4-yl)-N-methyl-N-(4-(piperidin-4-ylmethyl)phenyl)-benzamide (0.20 g). This material (0.10 g), K₂CO₃ (90 mg), and 1-fluoro-4-nitrobenzene (22 mg) were stirred ON in DMF (2 mL) at 100° C. The OL (EtOAc/water) was washed with brine, dried, concentrated, and purified by FC (heptane/EtOAc 1:1 to 0:1) to give Int. 2O4 4-(1-((4-bromo-1-methyl-1H-pyrrolo[2,3-b]pyridin-2-yl)methyl)-piperidin-4-yl)-N-methyl-N-(4-((1-(4-nitrophenyl)-piperidin-4-yl)methyl) phenyl)benzamide (98 mg).

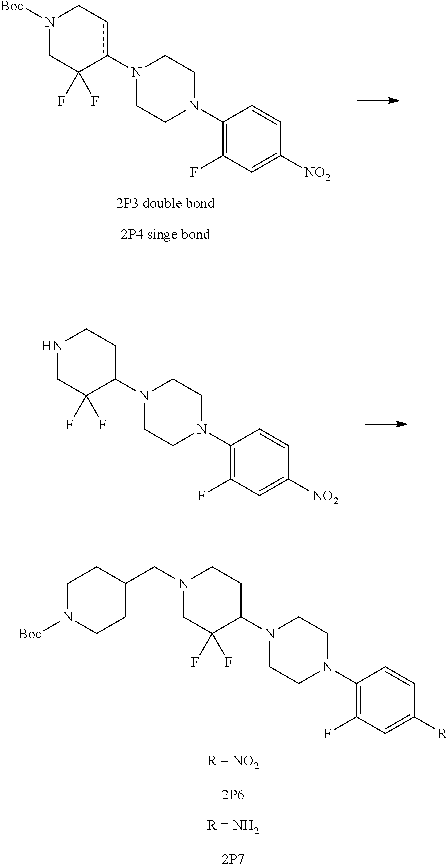

NaOAc (2.7 g) and 1-(2-fluoro-4-nitrophenyl) piperazine (3.5 g, HCl salt) were stirred 0.5 h in toluene/CAN/AcOH (8.8:3.8:1, 54 mL). tert-Butyl 3,3-difluoro-4-oxopiperidine-1-carboxylate (4.4 g) was added and stirring continued ON at 100° C. The filtrate after filtration was concentrated to give Int. 2P3 tert-butyl 3,3-difluoro-4-(4-(2-fluoro-4-nitro-phenyl) piperazin-1-yl)-3,6-dihydropyridine-1(2H)-carboxylate (7 g).

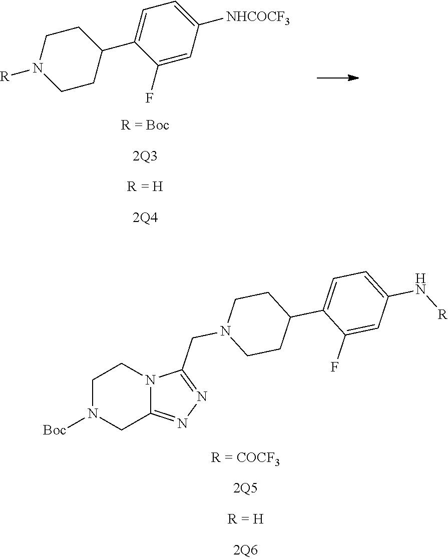

TFAA (2.5 mL) was added to a solution of tert-butyl 4-(4-amino-2-fluorophenyl)-piperidine-1-carboxylate (3.5 g) in toluene/Et₃N (6:1, 35 mL) at 0° C. before stirring ON at 0° C. to RT. The OL (EtOAc/water) was dried and concentrated to give Int. 2Q3 tert-butyl 4-(2-fluoro-4-(2,2,2-trifluoro-acetamido) phenyl) piperidine-1-carboxylate (4.5 g). This material was stirred 2 h in 2.4M HCl in dioxane (50 mL) at 0° C. to RT and concentrated to give Int. 2Q4 2,2,2-trifluoro-N-(3-fluoro-4-(piperidin-4-yl)-phenyl)-acetamide (3.3 g, HCl salt). Ints. 2Q4/2Q8 (0.65 g, HCl salt/0.79 g) were stirred ON in DMF/DIPEA (3:1, 8 mL). The OL (EtOAc/water) was washed with brine, dried, concentrated, and purified by FC (DCM/MeOH 95:5 to 9:1) to give Int. 2Q5 tert-butyl 3-((4-(2-fluoro-4-(2,2,2-trifluoro-acetamido) phenyl) piperidin-1-yl)methyl)-5,6-dihydro-[1,2,4]triazolo [4,3-a]pyrazine-7 (8H)-carboxylate (0.45 g). This material and K₂CO₃ (1.2 g) were stirred ON in MeOH/water (1:1, 8 mL). The OL (EtOAc/water) was washed with brine, dried, and concentrated to give Int. 2Q6 tert-butyl 3-((4-(4-amino-2-fluoro-phenyl) piperidin-1-yl)-methyl)-5,6-dihydro-[1,2,4]triazolo [4,3-a]pyrazine-7 (8H)-carboxylate (0.29 g).

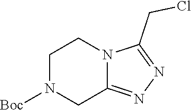 [SMILES: CC(C)(C)OC(=O)N1CCn2c(CCl)nnc2C1]

tert-Butyl 3-(hydroxymethyl)-5,6-dihydro-[1,2,4]-triazolo [4,3-a]pyrazine-7 (8H)-carboxylate (0.70 g) was stirred ON in DCM (10 mL), MsCl (0.4 mL), and Et₃N (1.5 mL) at 0° C. to RT. The OL (DCM/water) was dried and concentrated to give Int. 2Q8 tert-butyl 3-(chloromethyl)-5,6-dihydro-[1,2,4]triazolo [4,3-a]pyrazine-7 (8H)-carboxylate (0.81 g).

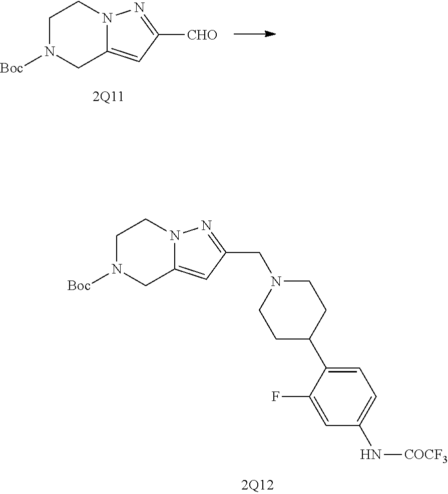

DMP (1.5 g) and tert-butyl 2-(hydroxymethyl)-6,7-dihydropyrazolo [1,5-a]pyrazine-5(4H)-carboxylate (0.45 g) were stirred 3 h in DCM (4 mL) at 0° C. to RT. The OL (DCM/aq. NaHCO₃) was washed with brine, dried, concentrated, and purified by FC (pentane/EtOAc 3:2 to 1:1) to give Int. 2Q11 tert-butyl 2-formyl-6,7-dihydro-pyrazolo-[1,5-a]pyrazine-5(4H)-carboxylate (0.22 g). Ints. 2Q11/2Q4 (0.2 g/0.26 g, HCl salt) were stirred 4 h in DCE/DIPEA (10:1, 2.2 mL). STAB (0.34 g) was added before stirring ON at 0° C. to RT. The OL (DCM/water) was washed with brine, dried, concentrated, and purified by FC (DCM/MeOH 98:2 to 95:5) to give Int. 2Q12 tert-butyl 2-((4-(2-fluoro-4-(2,2,2-trifluoro-acetamido) phenyl)-piperidin-1-yl)methyl)-6,7-dihydro-pyrazolo [1,5-a]pyrazine-5(4H)-carboxylate (0.17 g).

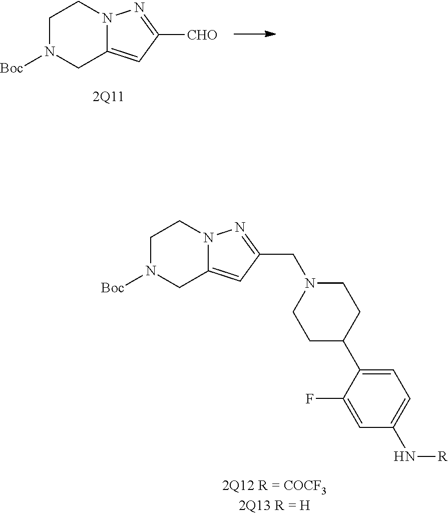

DMP (1.5 g) and tert-butyl 2-(hydroxymethyl)-6,7-dihydropyrazolo [1,5-a]pyrazine-5(4H)-carboxylate (0.45 g) were stirred 3 h in DCM (4 mL) at 0° C. to RT. The OL (DCM/aq. NaHCO₃) was washed with brine, dried, concentrated, and purified by FC (pentane/EtOAc 3:2 to 1:1) to give Int. 2Q11 tert-butyl 2-formyl-6,7-dihydro-pyrazolo-[1,5-a]pyrazine-5(4H)-carboxylate (0.22 g). Ints. 2Q11/2Q4 (0.2 g/0.26 g, HCl salt) were stirred 4 h in DCE/DIPEA (10:1, 2.2 mL). STAB (0.34 g) was added before stirring ON at 0° C. to RT. The OL (DCM/water) was washed with brine, dried, concentrated, and purified by FC (DCM/MeOH 98:2 to 95:5) to give Int. 2Q12 tert-butyl 2-((4-(2-fluoro-4-(2,2,2-trifluoro-acetamido) phenyl)-piperidin-1-yl)methyl)-6,7-dihydro-pyrazolo [1,5-a]pyrazine-5(4H)-carboxylate (0.17 g).

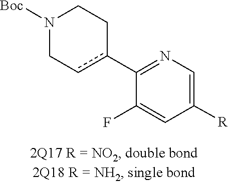

NaHCO₃ (1.4 g), 2-chloro-3-fluoro-5-nitro-pyridine (1.0 g), tert-butyl 4-(4,4,5,5-tetramethyl-1,3,2-dioxaborolan-2-yl)-3,6-dihydropyridine-1(2H)-carboxylate (1.93 g), and PdDPPFCl₂-DCM (0.37 g) were degassed in dioxane/water (5:1, 12 mL) before stirring ON at 100° C. and filtered. The OL (EtOAc/water) was dried, concentrated, and purified by FC (pentane/EtOAc 1:0 to 7:3) to give Int. 2Q17 tert-butyl 3-fluoro-5-nitro-3′, 6′-dihydro-[2,4′-bipyridine]-1′ (2′H)-carboxylate (0.6 g). Int. 2Q17 (7.0 g) and 10% Pd/C (2 g) were stirred 8 h in THF/EtOAc (1:1, 80 mL) under a H₂ atmosphere (60 psi), filtered, and concentrated to give Int. 2Q18 tert-butyl 4-(5-amino-3-fluoropyridin-2-yl) piperidine-1-carboxylate (6 g).

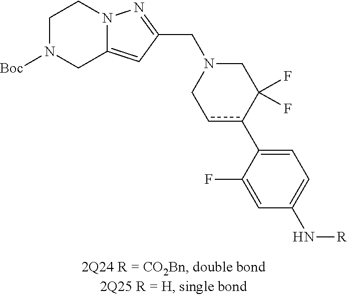

Ints. 2Q11/2M9 (1.2 g/2.0 g) were stirred 3 h in DCE/DIPEA (5:1, 24 mL) at 0° C. to RT. STAB (3.1 g) was added at 0° C. before stirring ON at 0° C. to RT. The OL (DCM/MeOH (9:1)/water) was washed with brine, dried, concentrated, and purified by FC (pentane/EtOAc 9:1 to 2:3) to give Int. 2Q24 tert-butyl 2-((4-(4-(((benzyloxy) carbo-nyl)amino)-2-fluorophenyl)-3,3-difluoro-3,6-dihydro-pyridin-1(2H)-yl)methyl)-6,7-dihydropyrazolo[1,5-a]-pyrazine-5(4H)-carboxylate (1.5 g).

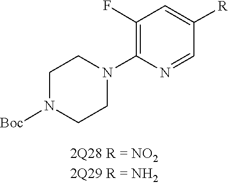

2-Chloro-3-fluoro-5-nitropyridine (4.0 g), tert-butyl piperazine-1-carboxylate (4.2 g), and K₂CO₃ (9.4 g) were stirred ON in DMF (7 mL) at 85° C. The OL (EtOAc/water) was washed with brine, dried, concentrated, and triturated in pentane and dried to give Int. 2Q28 tert-butyl 4-(3-fluoro-5-nitropyridin-2-yl)-piperazine-1-carboxylate (7 g). Int. 2Q28 (1.7 g) was hydrogenated ON using 10% Pd/C (0.5 g) in dioxane/THF (8 mL), filtered, concentrated, and triturated in pentane to give Int. 2Q29 tert-butyl 4-(5-amino-3-fluoropyridin-2-yl) piperazine-1-carboxylate (1.4 g).

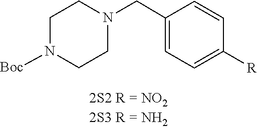

1-(Chloromethyl)-4-nitrobenzene (0.56 g), tert-butyl piperazine-1-carboxylate (0.50 g), and K₂CO₃ (0.75 g) were stirred 2 h in DMF (5 mL) at 70° C. The OL (EtOAc/water) was washed with brine, dried, and concentrated. The residue was purified by FC (heptane/EtOAc 9:1 to 0:1) to give Int. 2S2 tert-butyl 4-(4-nitro-benzyl) piperazine-1-carboxylate (0.83 g). Int. 2S2 (0.2 g), zinc powder (0.19 g), and NH₄Cl (0.15 g) were stirred 1 h in THF/water (1:1, 12 mL) and filtered. The OL (EtOAc/water) was washed with brine, dried, concentrated, and purified by FC (heptane/EtOAc 4:1 to 0:1) to give Int. 2S3 tert-butyl 4-(4-aminobenzyl)-piperazine-1-carboxylate (70 mg).

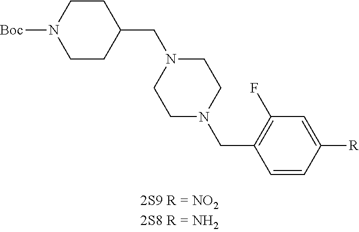

2-Fluoro-4-nitrobenzaldehyde (0.10 g), Int. 3E5 (0.25 g), and STAB (0.38 g) were stirred ON in DCE/DIPEA (19:1, 4.2 mL). STAB (0.10 g) and Int. 3E5(40 mg) were added and stirring continued 2 h. The mixture was concentrated and purified by FC (heptane/EtOAc 1:1 to 0:1) to give Int. 2S9 tert-butyl 4-[[4-[(2-fluoro-4-nitro-phenyl)methyl]piperazin-1-yl]methyl]piperidine-1-carboxylate (0.18 g). This material, NH₄Cl (45 mg), iron powder (0.16 g) were stirred 1.5 h in EtOH/water (4:1, 10 mL) at 80° C. The OL (EtOAc/water) was washed with brine, dried, concentrated, and purified by FC to give Int. 2S8 tert-butyl 4-[4-[(4-amino-2-fluoro-phenyl)methyl]piperazin-1-yl]methyl]piperidine-1-carboxylate (0.15 g).

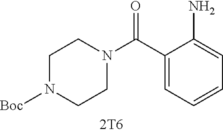 [SMILES: CC(C)(C)OC(=O)N1CCN(C(=O)c2ccccc2N)CC1]

2H-Benzo [d] [1,3]oxazine-2,4 (1H)-dione (1.0 g) and tert-butyl piperazine-1-carboxylate (1.1 g) were stirred 2 h in dioxane (9 mL) at 90° C. The OL (EtOAc/water) was washed with sat. aq. NH₄Cl and brine, dried, and concentrated, and purified by FC to give Int. 2T6 tert-butyl 4-(2-amino-benzoyl)-piperazine-1-carboxylate (1.8 g).

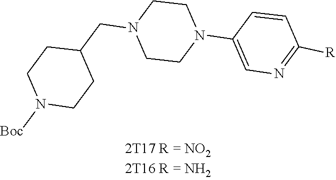

5-Fluoro-2-nitropyridine (0.13 g), Int. 3E5 (0.27 g), K₂CO₃ (0.30 g) were stirred ON in DMF (4 mL) at 60° C. The OL (water/EtOAc) was washed with brine, dried, concentrated, and purified by FC (heptane to EtOAc) to give Int. 2T17 tert-butyl 4-((4-(6-nitropyridin-3-yl) piperazin-1-yl)methyl)piperidine-1-carboxylate (0.26 g). Int. 2T17 (0.25 g) was hydrogenated ON using 10% Pd/C (80 mg) in MeOH/EtOAc (2:1, 15 mL), filtered, concentrated, and purified by FC (heptane/EtOAc/DCM/MeOH 1:0:0:0 to 0:9:0.5:0.5) to give Int. 2T16 tert-butyl 4-((4-(6-aminopyridin-3-yl) piperazin-1-yl)methyl)piperidine-1-carboxylate (0.22 g).

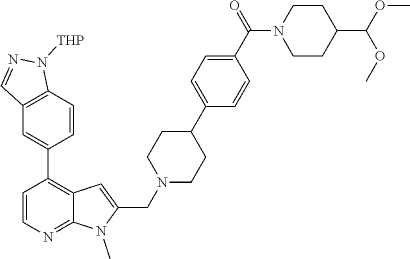

Int. 3A2 (0.50 g, HCl salt) and HATU (0.49 g) were stirred 0.3 h in DMF/DIPEA (20:1, 10.5 mL). 4-(Dimethoxy-methyl)piperidine (0.16 g) was added and stirring continued ON. The OL (water/EtOAc) was washed with brine, dried, concentrated, and purified by FC (DCM/MeOH 1:0 to 95:5) to give Int. 3D4 (4-(dimethoxy-methyl)-piperidin-1-yl) (4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)phenyl) methanone (0.20 g).

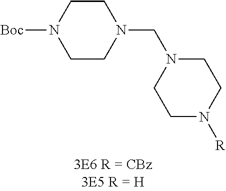

tert-Butyl 4-formylpiperidine-1-carboxylate (25 g) and benzyl piperazine-1-carboxylate (28.4 g) were stirred 4 h in DCE/DIPEA (4.1:1, 311 mL). STAB (49.5 g) was added and stirring continued ON at 0° C. to RT ON. The OL (water/DCM) was washed with brine, dried, concentrated, and purified by FC (pentane/EtOAc 1:1) to give Int. 3E6 benzyl piperazine-1-carboxylate (70 g). This material was hydrogenated ON using 10% Pd/C (13 g) in EtOH (1 L), filtered, and concentrated to give Int. 3E5 tert-butyl 4-(piperazin-1-ylmethyl)piperidine-1-carboxylate (40 g).

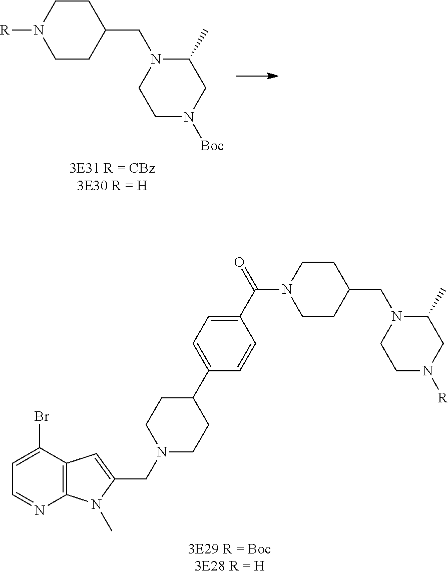

Benzyl 4-formylpiperidine-1-carboxylate (0.50 g) and tert-butyl (R)-3-methylpiperazine-1-carboxylate (0.40 g) were stirred 3 h in DCE/DIPEA (7:1, 5.7 mL). STAB (0.85 g) was added and stirring continued 3 h at 0° C. to RT. The OL (water/DCM) was dried, concentrated, and purified by FC (hexane/EtOAc 1:4) to give Int. 3E31 tert-butyl (R)-4-[(1-benzyloxycarbonyl-4-piperidyl)-methyl]-3-methyl-piperazine-1-carboxylate (0.65 g). This material was hydrogenated 8 h using and 5% Pd/C (0.1 g) in MeOH (5 mL). Filtration and concentration gave Int. 3E30 tert-butyl (3R)-3-methyl-4-(4-piperidylmethyl)-piperazine-1-carboxylate (0.35 g). Ints. 3E30/1AC4 (58 mg/70 mg) and HATU (87 mg) were stirred 3 h in DMF/DIPEA (28.1:1, 1.66 mL). The OL (EtOAc/water) was washed with brine, dried, concentrated, and HPLC-purified to give Int. 3E29 tert-butyl (R)-4-((1-(4-(1-((4-bromo-1-methyl-1H-pyrrolo[2,3-b]pyridin-2-yl)-methyl)piperidin-4-yl)benzoyl)piperidin-4-yl)methyl)-3-methyl-piperazine-1-carboxylate (60 mg). This material was stirred 0.5 h in DCM/TFA (2:1; 3 mL). The residue after concentration was SCX-purified to give Int. 3E28 (R)-(4-(1-((4-bromo-1-methyl-1H-pyrrolo [2,3-b] pyridin-2-yl)methyl)piperidin-4-yl)phenyl) (4-((2-methyl-piperazin-1-yl)methyl)-piperidin-1-yl) methanone (40 mg).

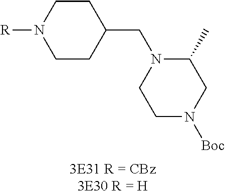

Benzyl 4-formylpiperidine-1-carboxylate (0.50 g) and tert-butyl (R)-3-methylpiperazine-1-carboxylate (0.40 g) were stirred 3 h in DCE/DIPEA (7:1, 5.7 mL). STAB (0.85 g) was added and stirring continued 3 h at 0° C. to RT. The OL (water/DCM) was dried, concentrated, and purified by FC (hexane/EtOAc 1:4) to give Int. 3E31 tert-butyl (R)-4-[(1-benzyloxycarbonyl-4-piperidyl)-methyl]-3-methyl-piperazine-1-carboxylate (0.65 g). This material was hydrogenated 8 h using and 5% Pd/C (0.1 g) in MeOH (5 mL). Filtration and concentration gave Int. 3E30 tert-butyl (3R)-3-methyl-4-(4-piperidylmethyl)-piperazine-1-carboxylate (0.35 g).

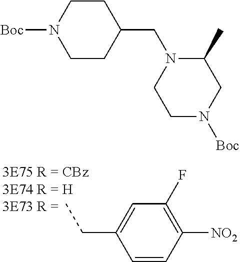

Benzyl(S)-3-methylpiperazine-1-carboxylate (2.0 g) and tert-butyl 4-formylpiperidine-1-carboxylate (1.8 g) were stirred 3 h in DCE (30 mL). STAB (3.6 g) was added and stirring continued ON. The OL (water/DCM) was dried, concentrated, and purified by FC (pentane/EtOAc 1:0 to 4:1) to give Int. 3E75 benzyl (3S)-4-[(1-tert-butoxy-carbonyl-4-piperidyl)methyl]-3-methyl-piperazine-1-carboxylate (3.0 g). This material was hydrogenated ON using 10% Pd/C (0.45 g) in MeOH (40 mL), filtered, and concentrated to give Int. 3E74 tert-butyl 4-[(2S)-2-methylpiperazin-1-yl]methyl]piperidine-1-carboxylate (1.9 g). Int. 3E74 (5.0 g) and 3-fluoro-4-nitrobenzaldehyde (3.0 g) were stirred 3 h in DCE/DIPEA (5.8:1, 59 mL). STAB (7.1 g) was added at 0° C. and stirring continued ON at 0° C. to RT. The OL (water/DCM) was dried, concentrated, and purified by FC (pentane/EtOAc 1:0 to 3:2) to give Int. 3E73 tert-butyl(S)-4-((4-(3-fluoro-4-nitrobenzyl)-2-methylpiperazin-1-yl)methyl)piperidine-1-carboxylate (1.8 g).

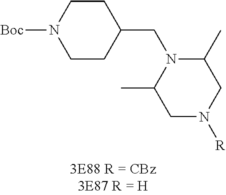

Benzyl 3,5-cis-dimethylpiperazine-1-carboxylate (1.5 g) and tert-butyl 4-formylpiperidine-1-carboxylate (1.7 g) were stirred ON in DCE/DIPEA (8.6:1, 6.7 mL) at 0° C. to RT. STAB (4.5 g) was added and stirring continued ON at 0° C. to RT. The OL (water/DCM) was washed with brine, dried and, concentrated. The residue was purified by FC (pentane/EtOAc 7:3 to 2:3) to give Int. 3E88 benzyl 4-((1-(tert-butoxycarbonyl)-piperidin-4-yl)-methyl)-3,5-cis-dimethylpiperazine-1-carboxylate (1.75 g). 1.7 g of this material was hydrogenated ON using 10% Pd/C (0.5 g) in EtOAc (10 mL), filtered, and concentrated to give Int. 3E87 tert-butyl 4-[[2,6-cis-dimethyl-piperazin-1-y]] methyl]-piperidine-1-carboxylate (1.1 g).

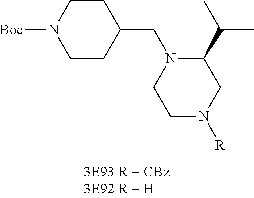

Benzyl(S)-3-iso-propylpiperazine-1-carboxylate (2.0 g, HCl salt) and tert-butyl 4-formylpiperidine-1-carboxylate (1.6 g) were stirred 4 h in DCE/DIPEA (4.5:1, 24.4 mL). STAB (2.8 g) was added and stirring continued 4 h. The OL (water/DCM) was washed with brine, dried, concentrated, and purified by FC (pentane/EtOAc 1:0 to 4:1) to give Int. 3E93 benzyl (3S)-4-[(1-tert-butoxycarbonyl-4-piperidyl)methyl]-3-iso-propyl-piperazine-1-carboxylate (1.9 g). 1.0 g of this material was hydrogenated ON using 10% Pd/C (0.2 g) in THF/EtOAc (1:1; 20 mL), filtered, and concentrated to give Int. 3E92 tert-butyl 4-[[(2S)-2-iso-propylpiperazin-1-yl]methyl]piperidine-1-carboxylate (0.6 g).

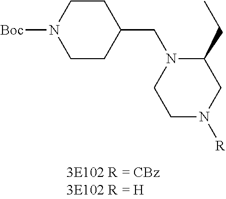

Benzyl (3S)-3-ethylpiperazine-1-carboxylate (2.66 g) and Int. and tert-butyl 4-formylpiperidine-1-carboxylate (2.51 g) were stirred 4 h in DCE/DIPEA (2.2:1, 29.3 mL). STAB (4.5 g) was added and stirring continued ON. The OL (water/DCM) was washed with brine, dried and, concentrated to give Int. 3E102 benzyl (3S)-4-[(1-tert-butoxy-carbonyl-4-piperidyl)methyl]-3-ethyl-piperazine-1-carboxylate (3.4 g). 0.73 g of this material and 10% Pd/C (0.18 g) were hydrogenated 4 h in THF/EtOAc (1:2.3, 20 mL), filtered, and concentrated to give Int. 3E101 tert-butyl 4-[(2S)-2-ethylpiperazin-1-yl]methyl]piperidine-1-carboxylate (0.5 g).

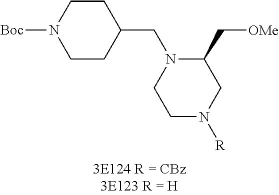

Benzyl (3R)-3-(methoxymethyl)piperazine-1-carboxylate (90 mg, HCl salt) and tert-butyl 4-formyl-piperidine-1-carboxylate (70 mg) were stirred 4 h in DCE/DIPEA (11.5:1, 3.3 mL). STAB (127 mg) was added and stirring continued ON. The OL (water/DCM) was washed with brine, dried and, concentrated. The residue was purified by FC (DCM/MeOH 95:5 to 9:1) to give Int. 3E124 benzyl (3R)-4-[(1-tert-butoxy-carbonyl-4-piperidyl)-methyl]-3-(methoxymethyl)piperazine-1-carboxylate (50 mg). Int. 3E124 (0.6 g) was hydrogenated 4 h using 10% Pd/C (0.35 g) in THF/EtOAc (1:1; 40 mL), filtered, concentrated, and triturated in pentane to give Int. 3E123 tert-butyl 4-[(2R)-2-(methoxymethyl)piperazin-1-yl]methyl]piperidine-1-carboxylate (0.5 g).

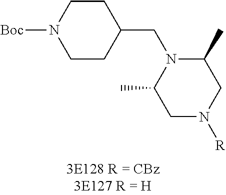

Benzyl (3S,5S)-3,5-dimethylpiperazine-1-carboxylate (1.2 g, HCl salt) and tert-butyl 4-formyl-piperidine-1-carboxylate (1.1 g) were stirred 4 h in DCE/DIPEA (1:1.9, 5.7 mL). STAB (1.8 g) was added at 0° C. and stirring continued ON at 0° C. to RT. The OL (water/DCM) was washed with brine, dried and, concentrated. The residue was purified by FC (pentane/EtOAc 7:3) to give Int. 3E128 benzyl (3S,5S)-4-[(1-tert-butoxy-carbonyl-4-piperidyl)-methyl]-3,5-dimethyl-piperazine-1-carboxylate (1.1 g). This material was hydrogenated 4 h using 10% Pd/C (0.3 g) in MeOH (35 mL), filtered, concentrated, and triturated in MeOH to give Int. 3E127 tert-butyl 4-[[(2S,6S)-2,6-dimethylpiperazin-1-yl]-methyl]piperidine-1-carboxylate (0.51 g).

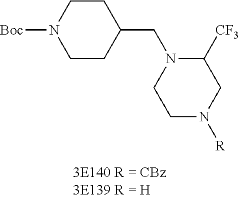

Benzyl 3-(trifluoromethyl)piperazine-1-carboxylate hydrochloride (2.4 g) and tert-butyl 4-formyl-piperidine-1-carboxylate (1.4 g) were stirred 4 h in DCE/DIPEA (6.4:1, 29 mL). STAB (3.1 g) was added at 0° C. and stirring continued ON at 0° C. to RT. The OL (water/DCM) was washed with brine, dried and, concentrated to give Int. 3E140 benzyl 4-((1-(tert-butoxycarbonyl)-piperidin-4-yl)methyl)-3-(trifluoro-methyl)piperazine-1-carboxylate (1.6 g). This material was hydrogenated ON using 10% Pd/C (0.3 g) in EtOAc/water (7:3; 10 mL), filtered, and concentrated to give Int. 3E139 tert-butyl 4-((2-(trifluoromethyl)-piperazin-1-yl)methyl)-piperidine-1-carboxylate (1.1 g).

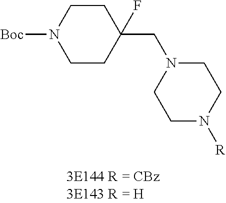

Benzyl piperazine-1-carboxylate (4.3 g) and tert-butyl 4-fluoro-4-formyl-piperidine-1-carboxylate (3.5 g) were stirred 3 h in DCE/DIPEA (3.6:1, 51 mL)). STAB (6.4 g) was added and stirring continued ON at 0° C. to RT. The OL (water/DCM) was washed with brine, dried, concentrated, and purified by FC (pentane/EtOAc 1:0 to 4:1) to give Int. 3E144 benzyl 4-((1-(tert-butoxy-carbonyl)-4-fluoropiperidin-4-yl)methyl)piperazine-1-carboxylate (4.0 g). This material was hydrogenated ON using 10% Pd/C (1.5 g) in THF/EtOAc (1:1; 30 mL), filtered, a concentrated to give Int. 3E143 tert-butyl 4-fluoro-4-(piperazin-1-ylmethyl)piperidine-1-carboxylate (2.3 g).

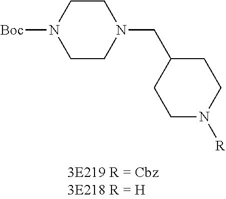

Benzyl 4-formylpiperidine-1-carboxylate (0.10 g) and tert-butyl piperazine-1-carboxylate (90 mg) were stirred 3 h in DCE/DIPEA (17.9:1, 5.3 mL) at 0° C. to RT. STAB (0.17 g) was added and stirring continued ON at 0° C. to RT. The OL (water/EtOAc) was dried and concentrated to give Int. 3E219 tert-butyl 4-[(1-benzyloxycarbonyl-4-piperidyl)-methyl]piperazine-1-carboxylate (0.10 g). Int.

3E219 (5.4 g) was hydrogenated ON using 10% Pd/C in EtOH (60 mL), filtered, and concentrated to give Int. 3E218 tert-butyl 4-(piperidin-4-ylmethyl)piperazine-1-carboxylate (3.4 g).

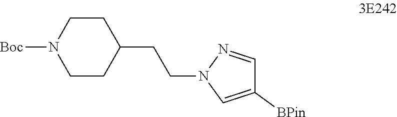

tert-Butyl 4-(2-hydroxyethyl)piperidine-1-carboxylate (0.26 g), 4-(4,4,5,5-tetramethyl-1,3,2-dioxaborolan-2-yl)-1H-pyrazole (0.20 g), and Ph₃P (0.3 g) were dissolved in THF (5 mL) at 0° C. DIAD (0.22 mL) was added at 3-6° C. and stirring was continued 2 h at RT. The residue after concentration was purified by FC (heptane/EtOAc 1:1 and DCM/EtOAc 0:1 to 2:3) to give Int. 3E242 tert-butyl 4-(2-(4-(4,4,5,5-tetramethyl-1,3,2-dioxaborolan-2-yl)-1H-pyrazol-1-yl)ethyl)piperidine-1-carboxylate (0.11 g).

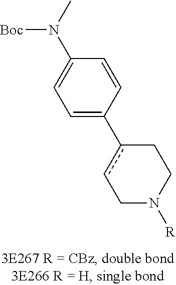

Benzyl 4-(4,4,5,5-tetramethyl-1,3,2-dioxaborolan-2-yl)-3,6-dihydropyridine-1(2H)-carboxylate (7.1 g), tert-butyl (4-bromophenyl) (methyl) carbamate (5.0 g), NaHCO₃ (4.3 g), and PdDPPFCl₂-DCM (0.7 g) were degassed in dioxane/water (9:1; 50 mL) and stirred ON at 100° C. and filtered. The OL (water/EtOAc) was washed with brine, dried, concentrated, and purified by FC (pentane/EtOAc 9:1 to 88:12) to give Int. 3E267 benzyl 4-(4-((tert-butoxy-carbonyl) (methyl)amino)phenyl)-3,6-dihydropyridine-1(2H)-carboxylate (4.5 g). 1.0 g of this material was hydrogenated ON using 10% Pd/C (0.5 g) in MeOH (12 mL), filtered, and concentrated to give Int. 3E266 tert-butyl methyl (4-(piperidin-4-yl)phenyl) carbamate (0.6 g).

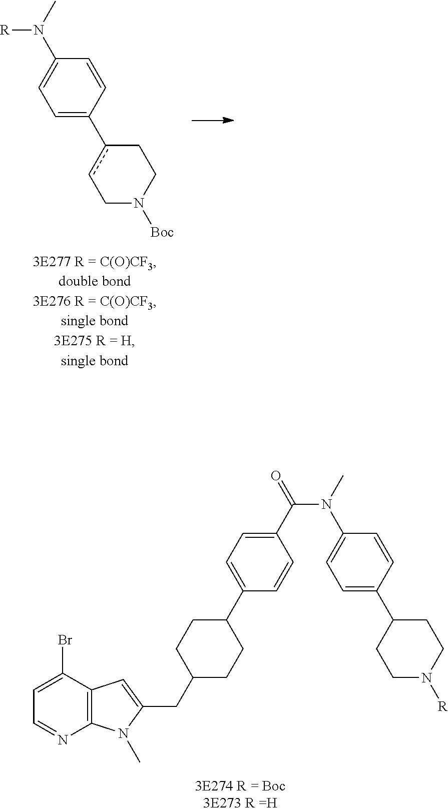

N-(4-bromophenyl)-2,2,2-trifluoro-N-methylacetamide (3.5 g), PdDPPFCl₂-DCM (0.44 g), tert-butyl 4-(4,4,5,5-tetramethyl-1,3,2-dioxaborolan-2-yl)-3,6-dihydropyridine-1(2H)-carboxylate (3.3 g), and NaHCO₃ (1.8 g) were degassed in dioxane/water (10:1, 34 mL) and stirred ON at 100° C. and filtered. The OL (EtOAc/water) was washed with brine, dried, and concentrated. The residue was mixed with two additional batches prepared similarly and purified by FC (pentane/EtOAc 9:1 to 85:15) to give Int. 3E277 tert-butyl 4-(4-(2,2,2-trifluoro-N-methylacetamido) phenyl)-3,6-dihydropyridine-1(2H)-carboxylate (8.3 g). 3.0 g of this material was hydrogenated ON using 10% Pd/C in MeOH (30 mL), filtered, concentrated, and purified by FC (pentane/EtOAc 9:1 to 85:15) to give Int. 3E276 tert-butyl 4-(4-(2,2,2-trifluoro-N-methylacetamido) phenyl) piperidine-1-carbo-xylate (2.5 g). 0.8 g of this material and K₂CO₃ (0.7 g) were stirred 3 h in MeOH (7 mL) and concentrated. The OL (water/DCM) was dried and concentrated to give Int. 3E275 tert-butyl 4-(4-(methylamino)phenyl)-piperidine-1-carboxylate (0.5 g). Ints. 1AC4/3E275 (0.5 g/0.58 g) and HATU (0.67 g) were stirred ON in DMF/DIPEA (24.5:1, 15.6 mL) and diluted with water to precipitate Int. 3E274 tert-butyl 4-(4-(4-(1-((4-bromo-1-methyl-1H-pyrrolo-[2,3-b]pyridin-2-yl)methyl)piperidin-4-yl)-N-methyl-benzamido)-phenyl) piperidine-1-carboxylate (0.6 g). 0.18 g of this material was stirred 0.5 h in DCM/TFA (2:1; 1.5 mL), concentrated, and SCX-purified to give Int. 3E273 4-[1-[(4-bromo-1-methyl-pyrrolo[2,3-b]pyridin-2-yl)methyl]-4-piperidyl]-N-methyl-N-[4-(4-piperidyl)phenyl]-benzamide (0.15 g).

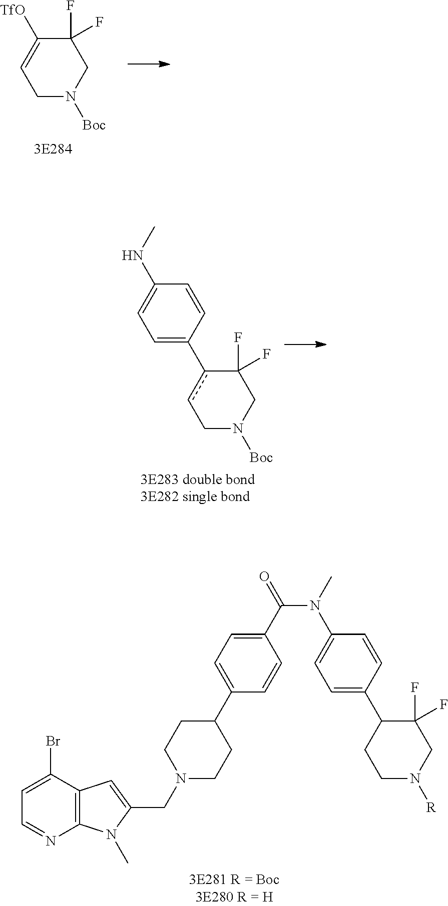

Tf₂O (1.6 g) was added to solution of tert-butyl 3,3-difluoro-4-oxopiperidine-1-carboxylate (0.88 g) in DCM/Et₃N (5:1, 9.6 mL) at 0° C. The mixture was stirred 0.5 h at 0° C., quenched with water, dried, and concentrated. The residue was combined with another batch prepared similarly on 4 g scale and purified by FC (pentane/EtOAc 9:1 to 4:1) to give Int. 3E284 tert-butyl 3,3-difluoro-4-(trifluoromethyl-sulfonyloxy)-2,6-dihydro-pyridine-1-carbo-xylate (1.5 g). 0.74 g of this material, N-methyl-4-(4,4,5,5-tetramethyl-1,3,2-dioxaborolan-2-yl) aniline (0.59 g), Na₂CO₃ (0.64 g), and PdDPPFCl₂-DCM (0.19 g) were degassed in dioxane/water (4:1, 15 mL) and stirred ON at 55° C. and filtered. The OL (EtOAc/water) dried and concentrated. The residue was purified by FC (pentane/EtOAc 7:3 to 3:2) and by HPLC to give Int. 3E283 tert-butyl 3,3-difluoro-4-[4-(methylamino)phenyl]-2,6-dihydro-pyridine-1-carboxylate (0.4 g). This material was hydrogenated ON using 10% Pd/C (0.15 g) in EtOAc/THF (1:1, 6 mL), filtered, and concentrated to give Int. 3E282 tert-butyl 3,3-difluoro-4-[4-(methylamino)-phenyl]-piperidine-1-carboxylate (0.34 g). Ints. 1AC4/3E282 (87 mg/60 mg) and HATU (84 mg) were stirred ON in DMF/DIPEA (6.3:1, 2.3 mL). HATU (70 mg) and DIPEA (0.16 mL) added and stirring continued ON at 50° C. HATU (70 mg) and DIPEA (0.16 mL) were added and stirring continued ON at 50° C. The OL (EtOAc/water) was concentrated and purified by FC (heptane/EtOAc 1:0 to 0:1) and by HPLC to give Int. 3E281 tert-butyl 4-[4-[4-[1-[(4-bromo-1-methyl-pyrrolo[2,3-b]pyridin-2-yl)methyl]-4-piperidyl]benzoyl]-methyl-amino]phenyl]-3,3-difluoro-piperidine-1-carboxylate (82 mg). A solution of this material in DCM/TFA (1:1; 2 mL) was stirred 0.5 h, concentrated and SCX-purified to give Int. 3E280 4-[1-[(4-bromo-1-methyl-pyrrolo-[2,3-b]pyridin-2-yl)methyl]-4-piperidyl]-N-[4-(3,3-difluoro-4-piperidyl)phenyl]-N-methyl-benzamide (52 mg).

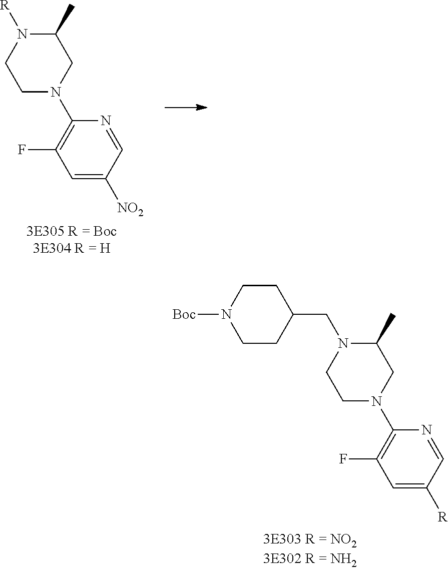

2-Chloro-3-fluoro-5-nitro-pyridine (0.50 g), tert-butyl (R)-2-methylpiperazine-1-carboxylate (0.62 g), and K₂CO₃ (1.6 g) were stirred ON in ACN (20 mL) at 85° C., concentrated, and triturated in water to give Int. 3E305 tert-butyl (2R)-4-(3-fluoro-5-nitro-2-pyridyl)-2-methyl-piperazine-1-carboxylate (0.84 g). This material was stirred 6 h in DCM/4M HCl in dioxane (1:2; 15 mL) at 0° C. and concentrated. The residue was triturated in Et₂O/pentane to give Int. 3E304 (3R)-1-(3-fluoro-5-nitro-2-pyridyl)-3-methyl-piperazine (0.65 g, HCl salt). This material and tert-butyl 4-formylpiperidine-1-carboxylate (0.42 g) were stirred ON in DCE/DIPEA (6.5:1, 17.3 mL). STAB (1.9 g) was added and stirring was continued 6 h at 0° C. to RT. The OL (water/DCM) was dried, concentrated, and triturated with Et₂₀ and dried to give Int. 3E303 tert-butyl 4-[(2R)-4-(3-fluoro-5-nitro-2-pyridyl)-2-methyl-piperazin-1-yl]methyl]piperidine-1-carboxylate (0.85 g). This material was hydrogenated ON using 10% Pd/C (0.4 g) in THF/EtOAc (1:2; 15 mL), filtered, and concentrated to give Int. 3E302 tert-butyl 4-[(2R)-4-(5-amino-3-fluoro-2-pyridyl)-2-methyl-piperazin-1-yl]methyl]piperidine-1-carboxylate (0.65 g).

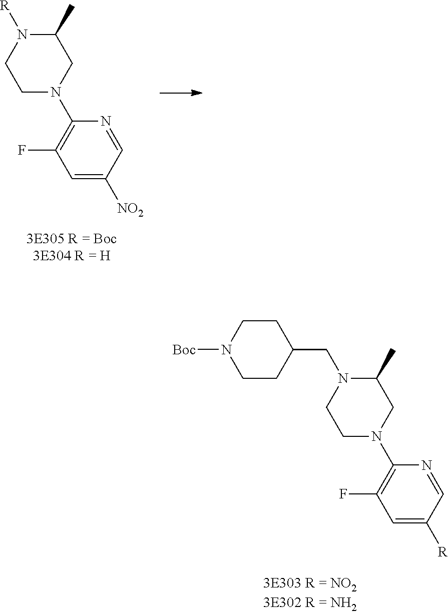

2-Chloro-3-fluoro-5-nitro-pyridine (0.50 g), tert-butyl (R)-2-methylpiperazine-1-carboxylate (0.62 g), and K₂CO₃ (1.6 g) were stirred ON in ACN (20 mL) at 85° C., concentrated, and triturated in water to give Int. 3E305 tert-butyl (2R)-4-(3-fluoro-5-nitro-2-pyridyl)-2-methyl-piperazine-1-carboxylate (0.84 g). This material was stirred 6 h in DCM/4M HCl in dioxane (1:2; 15 mL) at 0° C. and concentrated. The residue was triturated in Et₂O/pentane to give Int. 3E304 (3R)-1-(3-fluoro-5-nitro-2-pyridyl)-3-methyl-piperazine (0.65 g, HCl salt). This material and tert-butyl 4-formylpiperidine-1-carboxylate (0.42 g) were stirred ON in DCE/DIPEA (6.5:1, 17.3 mL). STAB (1.9 g) was added and stirring was continued 6 h at 0° C. to RT. The OL (water/DCM) was dried, concentrated, and triturated with Et₂O and dried to give Int. 3E303 tert-butyl 4-[[(2R)-4-(3-fluoro-5-nitro-2-pyridyl)-2-methyl-piperazin-1-yl]methyl]piperidine-1-carboxylate (0.85 g). This material was hydrogenated ON using 10% Pd/C (0.4 g) in THF/EtOAc (1:2; 15 mL), filtered, and concentrated to give Int. 3E302 tert-butyl 4-[(2R)-4-(5-amino-3-fluoro-2-pyridyl)-2-methyl-piperazin-1-yl]methyl]piperidine-1-carboxylate (0.65 g).

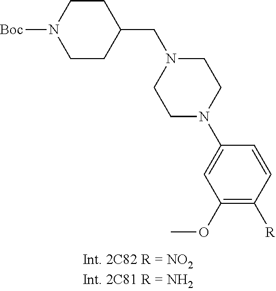

4-Fluoro-2-methoxy-1-nitro-benzene (12.5 g), K₂CO₃ (24 g), and tert-butyl 4-(piperazin-1-ylmethyl)-piperidine-1-carboxylate (10 g) were stirred 12 h in DMF (100 mL) at 80° C. The OL (water/EtOAc) was dried, concentrated, and purified by FC (DCM/MeOH 1:0 to 9:1) to give Int. 2C82 tert-butyl 4-((4-(3-methoxy-4-nitrophenyl) piperazin-1-yl)methyl)piperidine-1-carboxylate (20 g). Int. 2C82 (20 g) and 10% Pd/C (5 g) were stirred ON in MeOH (200 mL) at 70° C. under an atmosphere of hydrogen. The mixture was filtered and concentrated to give crude Int. 2C81 tert-butyl 4-((4-(4-amino-3-methoxyphenyl) piperazin-1-yl)methyl)-piperidine-1-carboxylate (15 g).

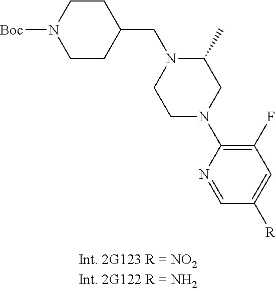

Int. 2F12 (18 g), 2-chloro-3-fluoro-5-nitropyridine (8 g), and K₂CO₃ (13 g) were stirred 16 h in ACN (60 mL) at 100° C. under MW conditions. The mixture was concentrated. The OL (water/EtOAc) was dried, concentrated, and purified by FC (PE/EtOAc 2:1) to give Int. 2G123 tert-butyl (R)-4-((4-(3-fluoro-5-nitro-pyridin-2-yl)-2-methylpiperazin-1-yl)methyl)piperidine-1-carboxylate (16 g). Int. 2G123 (16 g) and Pd/C 10% (5 g) were hydrogenated 16 h at 60 psi in MeOH (250 mL). The mixture was filtered and concentrated to give Int. 2G122 tert-butyl (R)-4-((4-(5-amino-3-fluoropyridin-2-yl)-2-methylpiperazin-1-yl)methyl)piperidine-1-carboxylate (13 g).

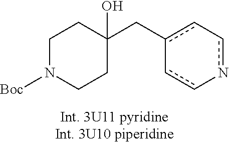

1.6M n-BuLi in hexane (200 mL) was added to a solution of 4-methylpyridine (26 mL) in THF (200 mL) at −78° C. and the mixture stirred for 30 min. tert-Butyl 4-oxopiperidine-1-carboxylate (59 g) in THF (100 mL) was added dropwise and stirring continued 0.5 h at −78° C. to RT. The OL (aq. NH₄Cl/EtOAc) was dried, concentrated, and purified by FC (PE/EtOAc 1:4 to 1:9) to give Int. 3U11 tert-butyl 4-hydroxy-4-(pyridin-4-ylmethyl)piperidine-1-carboxylate (30 g). Int. 3U11 (12 g) and PtO₂ (5.6 g) were hydrogenated at 100 psi for 16 h at 60° C. in EtOH (200 mL). The mixture was filtered and concentrated. The residue was stirred in aq. Na₂CO₃ and freeze-dried. The residue was dissolved in DCM, filtered, and concentrated to give Int. 3U10 tert-butyl 4-hydroxy-4-(piperidin-4-ylmethyl)-piperidine-1-carboxylate (10 g).

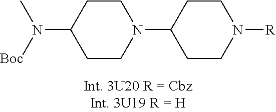

NaCNBH₃ (0.75 g), benzyl 4-oxopiperidine-1-carboxylate (2.2 g), and tert-butyl methyl (piperidin-4-yl) carbamate (2.0 g) were stirred 18 h in MeOH (30 mL). The mixture was concentrated. The OL (sat. aq. NaHCO/DCM) was dried, and concentrated to give Int. 3U20 benzyl 4-((tert-butoxy-carbonyl) (methyl)-amino)-[1,4′-bipiperidine]-1′-carboxylate (2.5 g). Int. 3U20 (2.5 g) and Pd/C (0.25 g) were hydrogenated 72 h at 735 psi in MeOH (30 mL) at 50° C. The mixture was filtered and concentrated to give Int. 3U19 tert-butyl [1,4′-bipiperidin]-4-yl (methyl) carbamate (1.8 g).

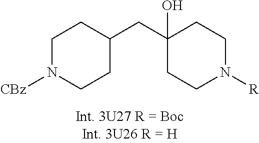

NaHCO₃ (19 g) and Int. 3U10 (10 g) were stirred 16 h in Et₂O/H₂O (1:1, 100 mL) and 50% benzyl chloro-formate in toluene (11 mL, added drop-wise) at 0° C. to RT. The OL (water/EtOAc) was dried, concentrated, and purified by FC (PE/EtOAc 4:1 to 1:1) to give Int. 3U27 tert-butyl 4-((1-((benzyloxy)-carbonyl) piperidin-4-yl)methyl)-4-hydroxypiperidine-1-carboxylate (8 g). Int. 3U27 (1 g) in HFP (15 mL) was heated under MW conditions for 16 h at 140° C. The mixture was combined with two other batches prepared similarly and concentrated to give Int. 3U26 benzyl 4-((4-hydroxypiperidin-4-yl)-methyl)piperidine-1-carboxylate (2.1 g).

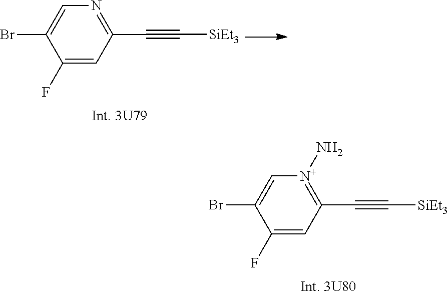

5-bromo-4-fluoro-2-((triethylsilyl) ethynyl)pyridine (11.6 g), Pd(PPh₃) 2Cl2 (1.35 g), and CuI (0.1 g) were stirred ON in Et₃N (ACN (2.1/70 mL) triethyl (ethynyl) silane (7.4 mL) at 0° C. to RT under an inert atmosphere. The mixture was concentrated. The OL (water/EtOAc) was dried, concentrated, and purified by FC (hexane/DCM 1:0 to 0:1) to give Int. 3U79 5-bromo-4-fluoro-2-((triethylsilyl)-ethynyl)-pyridine (5.0 g). tert-butyl ((mesitylsulfonyl)oxy) carbamate (10.0 g) was added in portions to TFA (18 mL) at 0° C. and stirred for 1.5 h. The mixture was poured onto ice to precipitate a solid which was dissolved in DCM (80 mL). The OL (water/DCM) was dried and filtered and added slowly to a solution of Int. 3U79 (5.0 g) in DCM (80 mL) at 0° C. and stirred ON at RT and concentrated to give Int. 3U80 1-amino-5-bromo-4-fluoro-2-((triethylsilyl) ethynyl)pyridin-1-ium (8.0 g, 2,4,6-trimethylbenzenesulfonate salt).

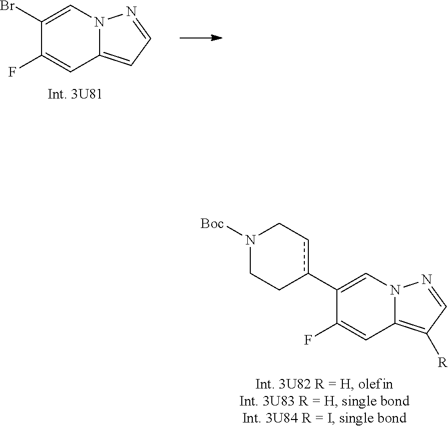

Ag₂CO₃ (5.0 g) and Int. 3U80 (8.0 g) were stirred 12 h in DMF (20 mL) at 0° C. to RT. The mixture was filtered. The OL (water/EtOAc) was dried, concentrated, and purified by FC (hexane/MTBE 1:0 to 0:1) to give Int. 3U81 6-bromo-5-fluoropyrazolo[1,5-a]pyridine (0.33 g). Int. 3U81 (0.33 g), tert-butyl 4-(4,4,5,5-tetramethyl-1,3,2-dioxaborolan-2-yl)-3,6-dihydropyridine-1(2H)-carboxylate (0.57 g), Cs₂CO₃ (0.64 g), and PdDPPFCl₂-DCM (50 mg) were stirred ON in dioxane/H₂O (4:1, 5 mL) at 90° C. under an inert atmosphere. The mixture was concentrated. The OL (water/EtOAc) was dried, concentrated, and HPLC-purified to give Int. 3U82 tert-butyl 4-(5-fluoropyrazolo[1,5-a]pyridin-6-yl)-3,6-dihydro-pyridine-1(2H)-carboxylate (0.31 g). Int. 3U82 (0.31 g) and Pd/C 5% wt. (0.1 g) were hydrogenated ON in MeOH (5 mL). The mixture was filtered and concentrated to give Int. 3U83 tert-butyl 4-(5-fluoropyrazolo[1,5-a]pyridin-6-yl) piperidine-1-carboxylate (0.27 g). NIS (0.19 g) and Int. 3U83 (0.27 g) were stirred 12 h in ACN (3 mL) at 0° C. to RT. The mixture was HPLC-purified to give Int. 3U84 tert-butyl 4-(5-fluoro-3-iodopyrazolo[1,5-a]pyridin-6-yl) piperidine-1-carboxylate (125 mg).

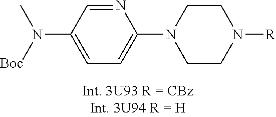

Pd₂ (dba) 3 (2.3 g), Xantphos (2.8 g), Int. 1Y⁴ (23 g), benzyl piperazine-1-carboxylate (23 g), and NaO^{t}Bu (12 g) were stirred 16 h in toluene (200 mL) at 100° C. under an inert atmosphere. The mixture was filtered. The OL (water/EtOAc) was dried, concentrated, and purified by FC (PE/EtOAc 1:1 to 2:5) to give Int. 3U93 benzyl 4-(5-((tert-butoxycarbonyl)(methyl)amino)pyridin-2-yl) piperazine-1-carboxylate. Int. 3U93 (13 g) and Pd/C 10% wet (4 g) were hydrogenated 5 h at 60 psi in MeOH (150 mL). The mixture was filtered and concentrated to give Int. 3U94 tert-butyl methyl (6-(piperazin-1-yl)pyridin-3-yl) carbamate (8 g).

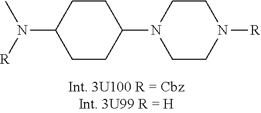

NaCNBH₃ (1.8 g), tert-butyl methyl (4-oxocyclohexyl) carbamate (5.0 g), benzyl piperazine-1-carboxylate (4.9 g) were stirred 18 h in MeOH (50 mL). The mixture was concentrated. The OL (sat. aq. NaHCO₃/DCM) was dried, and concentrated to give Int. 3U100 benzyl 4-(4-((tert-butoxy-carbonyl) (methyl)amino)cyclohexyl)-piperazine-1-carboxylate (6.1 g). Int. 3U100 (6.1 g) an Pd/C (0.61 g) were hydrogenated 72 h at 735 psi at 50° C. in MeOH (75 mL). The mixture was filtered and concentrated to give Int. 3U99 tert-butyl methyl (4-(piperazin-1-yl)cyclohexyl) carbamate (1.8 g).

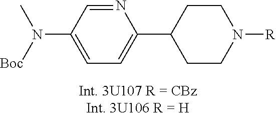

Int. 1Y⁴ (8.0 g), benzyl 4-(4,4,5,5-tetramethyl-1,3,2-dioxaborolan-2-yl)-3,6-dihydropyridine-1(2H)-carboxylate (14 g), PdDPPFCl₂-DCM (1.1 g), and NaHCO₃ (7.0 g) were stirred 16 h in THF/H₂O (8:1, 90 mL) at 100° C. under an inert atmosphere. The mixture was filtered. The OL (water/EtOAc) was dried, concentrated, and purified by FC (PE/EtOAc 4:1) to give Int. 3U107 benzyl 5-((tert-butoxy-carbonyl)(methyl)amino)-3′, 6′-dihydro-[2,4′-bipyridine]-1′ (2′H)-carboxylate (8.5). Int. 3U107 (2.5 g) and Pd/C 10% (0.10 g) were hydrogenated 16 h at 60 psi in MeOH (30 mL). The mixture was filtered and concentrated to give Int. 3U106 tert-butyl methyl (6-(piperidin-4-yl)pyridin-3-yl)-carbamate (2.0 g).

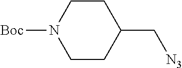 [SMILES: CC(C)(C)OC(=O)N1CCC(CN=[N+]=[N-])CC1]

NaN₃ (0.11 g) and tert-butyl 4-(bromomethyl)piperidine-1-carboxylate (0.40 g) were stirred ON in DMF (5 mL). The OL (water/EtOAc) was washed with brine, dried, and concentrated to give Int. 3T8 tert-butyl 4-(azidomethyl)piperidine-1-carboxylate (0.32 g).

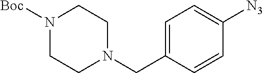 [SMILES: CC(C)(C)OC(=O)N1CCN(Cc2ccc(N=[N+]=[N-])cc2)CC1]

tert-Butyl 4-(4-bromobenzyl) piperazine-1-carboxylate (0.42 g), sodium azide (0.38 g), sodium ascorbate (40 mg), CuI (80 mg) and trans-N,N′-dimethylcyclohexane-1,2-diamine (0.12 mL) in EtOH/H₂O (7:3, 12 mL) were refluxed for 6 h under an inert atmosphere and concentrated. The OL (water/DCM) was concentrated and purified by FC (heptane/EtOAc 1:0 to 1:1) to give Int. TT23 tert-butyl 4-(4-azidobenzyl) piperazine-1-carboxylate (0.28 g).

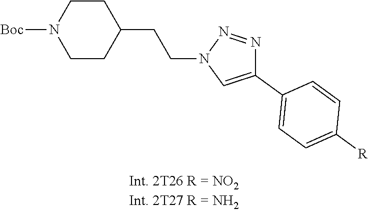

Sodium ascorbate (12 g) was added to a mixture of 1-ethynyl-4-nitrobenzene (3.0 g), tert-butyl 4-(2-azidoethyl)piperidine-1-carboxylate (5.2 g) and CuSO₄·H₂O (6.5 g) in ‘BuOH/H₂O (1:1, 100 mL), stirred for 16 h, filtered and concentrated to give Int. 2T26 tert-butyl 4-(2-(4-(4-nitrophenyl)-1H-1,2,3-triazol-1-yl)-ethyl)piperidine-1-carboxylate (4.5 g). Fe powder (2.7 g) and NH₄Cl (5.3 g) were added to a solution of Int. 2T26 (4.0 g) in EtOH/H₂O (5:1. 60 mL) and stirred for 3 h at 90° C. and filtered. The OL (brine/EtOAc) was dried, filtered and concentrated to give Int. 2T27 tert-butyl 4-(2-(4-(4-aminophenyl)-1H-1,2,3-triazol-1-yl)-ethyl)piperidine-1-carboxylate (3.2 g).

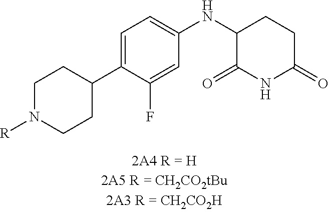

Int. 2A4 (0.22 g), tert-butyl 2-bromoacetate (0.16 g), and Cs₂CO₃ (0.71 g) were stirred ON in ACN (5 mL) and concentrated. The OL (water/DCM) was washed with brine, dried, concentrated, and was purified by FC (heptane/EtOAc 1:0 to 0:1) to give Int. 2A5 tert-butyl 2-(4-(4-((2,6-dioxopiperidin-3-yl)amino)-2-fluoro-phenyl) piperidin-1-yl)acetate (80 mg). This material was stirred 2 h in DCM/TFA (1:1, 8 mL) and concentrated to give Int. 2A3 2-(4-(4-((2,6-dioxopiperidin-3-yl)amino)-2-fluorophenyl) piperidin-1-yl) acetic acid (88 mg, TFA salt).

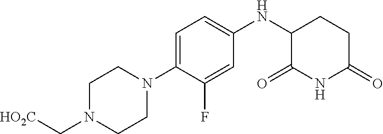 [SMILES: O=C(O)CN1CCN(c2ccc(NC3CCC(=O)NC3=O)cc2F)CC1]

Int. 2A6. 2-(4-(4-((2,6-dioxopiperidin-3-yl)amino)-2-fluorophenyl) piperazin-1-yl) acetic acid (121 mg, TFA salt) prepared similarly to Int. 2A3 from Int. 2A7.

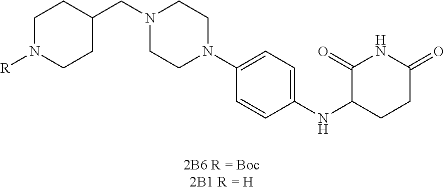

Int. 2B2 tert-butyl 4-((4-(4-nitrophenyl) piperazin-1-yl)methyl)piperidine-1-carboxylate (0.45 g) was prepared from 1-fluoro-4-nitrobenzene similarly to Int. 2I4. Int. 2B1 (20 mg, TFA salt) prepared similarly to Int. 2F10 from Int. 2B2.

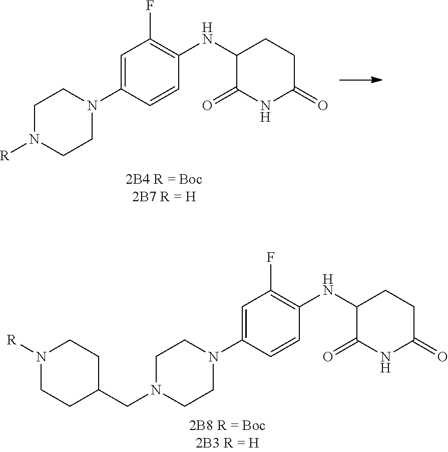

Int. 2B4 tert-butyl 4-(4-((2,6-dioxopiperidin-3-yl)amino)-3-fluorophenyl) piperazine-1-carboxylate (1.3 g) prepared similarly to Int. 2I4 from 2,4-difluoro-1-nitrobenzene. Int. 2B3 3-((2-fluoro-4-(4-(piperidin-4-ylmethyl)piperazin-1-yl)phenyl)amino)piperidine-2,6-dione (0.13 g, HCl salt) prepared similarly to Int. 2C11 from Int. 2B4.

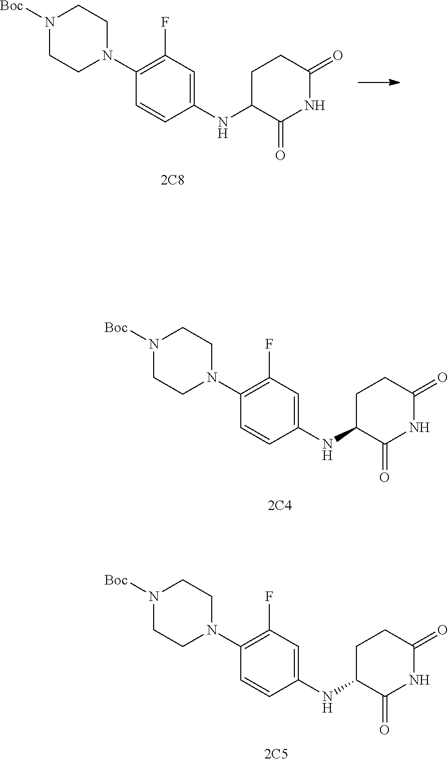 [SMILES: CC(C)(C)OC(=O)N1CCN(c2ccc(NC3CCC(=O)NC3=O)cc2F)CC1 ; CC(C)(C)OC(=O)N1CCN(c2ccc(N[C@H]3CCC(=O)NC3=O)cc2F)CC1 ; CC(C)(C)OC(=O)N1CCN(c2ccc(N[C@@H]3CCC(=O)NC3=O)cc2F)CC1]

Int. 2C7 (2 g), 3-bromo-piperidine-2,6-dione (2.6 g), and NaHCO₃ (2.3 g) were stirred ON at 80° C. in DMF (8 mL). The OL (water/EtOAc) was dried, concentrated, and purified by FC (pentane/EtOAc 1:0 to 1:4) to give Int. 2C8 tert-butyl 4-(4-((2,6-dioxo-piperidin-3-yl)-amino)-2-fluorophenyl)-piperazine-1-carboxylate (1.6 g). Int. 2C8 (1.4 g) was resolved by SFC using a Chiralpak IG 250×30 5 μm column using an eluent of 60% CO₂ and 40% 0.1% isopropyl amine in IPA/ACN (1:1) at a flow rate of 90 g/min and a back pressure of 100 bar at 30° C. to give Ints. 2C4 (0.45 g, first eluting isomer) and 2C5 (0.46 g, second eluting isomer) tert-butyl(S)-4-(4-((2,6-dioxopiperidin-3-yl)amino)-2-fluoro-phenyl) piperazine-1-carboxylate and tert-butyl (R)-4-(4-((2,6-dioxopiperidin-3-yl)amino)-2-fluoro-phenyl) piperazine-1-carboxylate. The absolute configurations of these compounds were not determined.

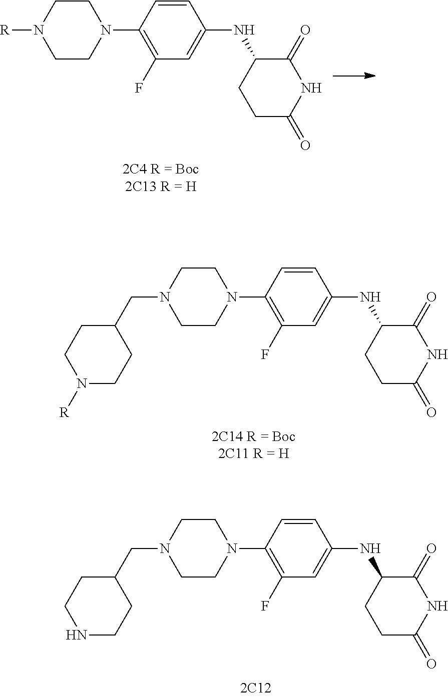

Int. 2C4 (0.10 g) was stirred 1 h in DCM/4M HCl in dioxane (2:1, 6 mL) at 0° C. to RT and concentrated. The residue was triturated in Et₂O to give Int. 2C13 (S)-3-((3-fluoro-4-(piperazin-1-yl)phenyl)amino)-piperidine-2,6-dione (80 mg). Int. 2C13 (35 mg) and tert-butyl 4-formylpiperidine-1-carboxylate (22 mg) were stirred 4 h in DCE/DIPEA (44:1, 4.1 mL). STAB (43 mg) was added and stirring continued ON before concentration. The OL (sat. aq. NaHCO/DCM) was concentrated and purified by FC (DCM/MeOH 1:1 to 9:1) to give Int. 2C14 tert-butyl(S)-4-((4-(4-((2,6-dioxopiperidin-3-yl)-amino)-2-fluoro-phenyl) piperazin-1-yl)methyl)-piperidine-1-carboxylate (35 mg). This material was stirred 1 h in DCM/4M HCl in dioxane (1:1, 2 mL) at 0° C. to RT and concentrated. The residue was triturated in Et₂O to give Int. 2C11 (15 mg, HCl salt) and Int. 2C12 was prepared similarly to from Int. 2C5. Ints. 2C11 and 2C12 (S)-3-((3-fluoro-4-(4-(piperidin-4-ylmethyl)-piperazin-1-yl)phenyl)amino)piperidine-2,6-dione and (R)-3-((3-fluoro-4-(4-(piperidin-4-ylmethyl)piperazin-1-yl)phenyl)amino)piperidine-2,6-dione for which the absolute configurations were not determined.

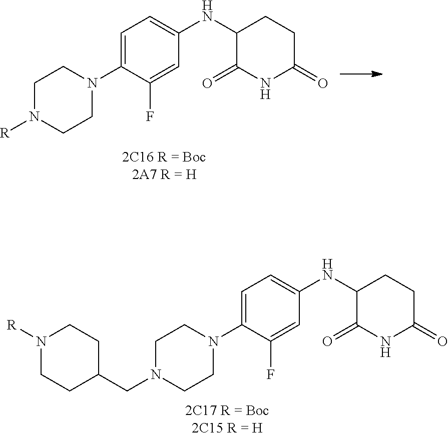

K₂CO₃ (1.34 g), tert-butyl piperazine-1-carboxylate (0.60 g), and 1,2-difluoro-4-nitro-benzene (0.52 g) were stirred ON in DMF (3 mL) at 70° C. The OL (water/EtOAc) was washed with 3M aq. CaCl₂), brine, concentrated, and purified by FC (heptane/EtOAc 1:0 to 7:3) to give Int. 2C16 tert-butyl 4-(4-((2,6-dioxo-piperidin-3-yl)amino)-2-fluorophenyl) piperazine-1-carboxylate (0.94 g). Int. 2C16 (0.60 g) was stirred 0.75 h in DCM/TFA (1:1, 12 mL). The mixture was concentrated to give Int. 2A7 3-((3-fluoro-4-(piperazin-1-yl)phenyl)-amino)-piperidine-2,6-dione (0.89 g, TFA salt). Int. 2A7 (0.12 g) and tert-butyl 4-formylpiperidine-1-carboxylate (43 mg) were stirred 2 h in ACN/DIPEA (17.6:1, 5.3 mL) at 66° C. The residue after concentration was HPLC-purified to give Int. 2C17 tert-butyl 4-((4-(4-((2,6-dioxo-piperidin-3-yl)-amino)-2-fluorophenyl)-piperazin-1-yl)-methyl)piperidine-1-carboxylate (77 mg). This material was stirred 5 h in DCM/TFA (2:1. 6 mL) and concentrated to give Int. 2C15 3-((3-fluoro-4-(4-(piperidin-4-ylmethyl)piperazin-1-yl)phenyl)amino)piperidine-2,6-dione (121 mg, TFA salt).

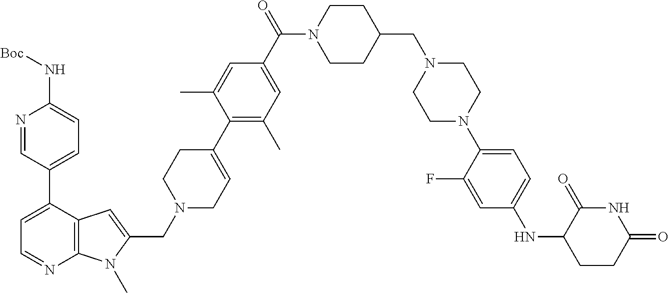 [SMILES: Cc1cc(C(=O)N2CCC(CN3CCN(c4ccc(NC5CCC(=O)NC5=O)cc4F)CC3)CC2)cc(C)c1C1=CCN(Cc2cc3c(-c4ccc(NC(=O)OC(C)(C)C)nc4)ccnc3n2C)CC1]

Ints. 3E41/2C15 (20/16 mg) and HATU (16 mg) were stirred 2 h in DCM/DIPEA/DMF (6:1:1.2, 0.41 mL). The OL (sat. aq. NaHCO₃/DCM) was concentrated and purified by FC (DCM/MeOH 1:0 to 96:4) to give Int. 2C18 tert-butyl (5-(2-((4-(4-(4-((4-(4-((2,6-dioxopiperidin-3-yl)amino)-2-fluorophenyl) piperazin-1-yl)methyl)piperidine-1-carbonyl)-2,6-dimethylphenyl)-3,6-dihydropyridin-1(2H)-yl)methyl)-1-methyl-1H-pyrrolo[2,3-b]pyridin-4-yl)pyridin-2-yl) carbamate (22 mg).

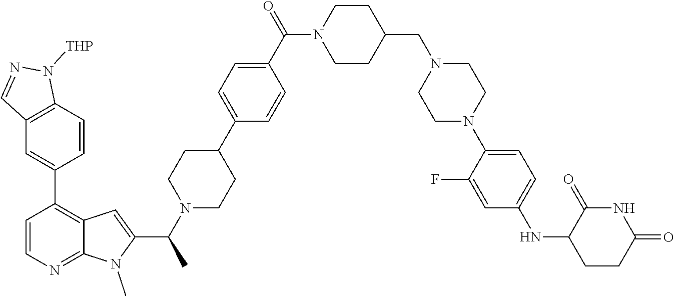

Int. 2C19 3-((3-fluoro-4-(4-((1-(4-(1-((1S)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoyl)piperidin-4-yl)methyl)piperazin-1-yl)phenyl)amino)-piperidine-2,6-dione (0.19 g) prepared similarly to Int. 2C18 from Ints. 2C54/2C15 (0.12 g/0.12 g, TFA salt).

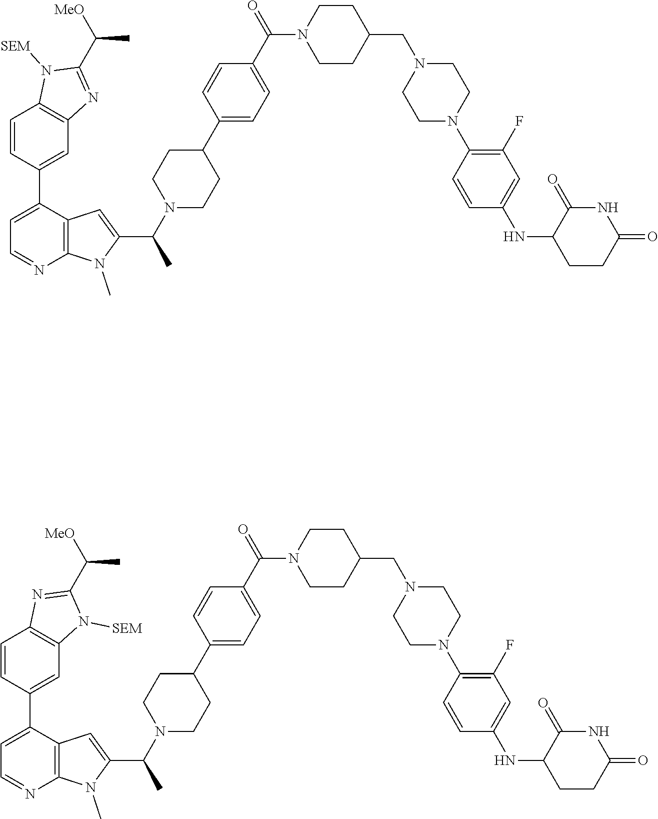 [SMILES: CO[C@@H](C)c1nc2cc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)ccc2n1COCC[Si](C)(C)C ; CO[C@@H](C)c1nc2ccc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)cc2n1COCC[Si](C)(C)C]

Int. 2C30/2C31 3-((3-fluoro-4-(4-((1-(4-(1-((S)-1-(4-(2-((S)-1-methoxyethyl)-1-((2-(trimethylsilyl)-ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperazin-1-yl)phenyl)amino)piperidine-2,6-dione and 3-((3-fluoro-4-(4-((1-(4-(1-((S)-1-(4-(2-((S)-1-methoxyethyl)-1-((2-(trimethylsilyl) ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)-piperidine-2,6-dione (0.30 g) prepared similarly to Int. 2C18 from Ints. 2C20/2C21/2C15 (0.20 g/0.29 g, HCl salt).

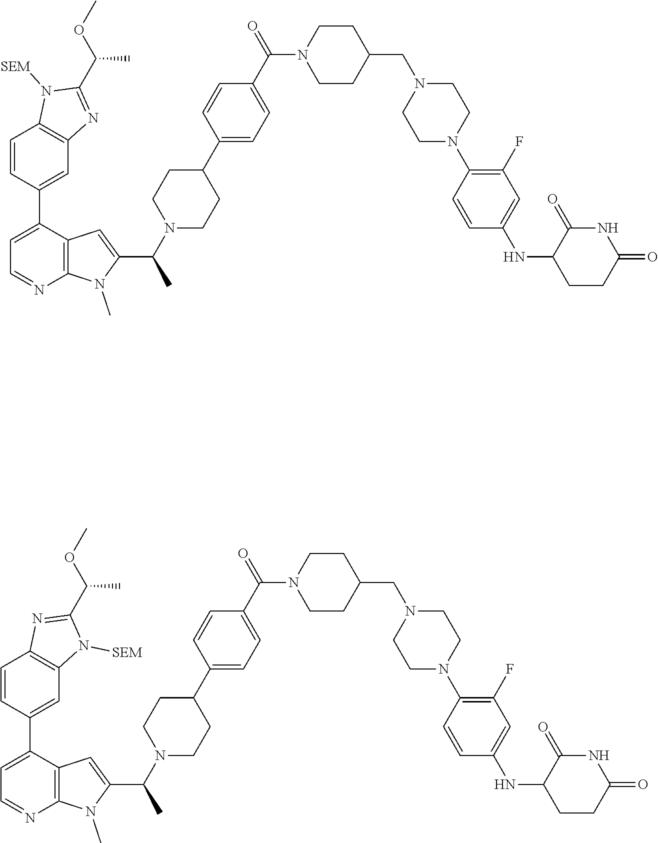 [SMILES: CO[C@H](C)c1nc2cc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)ccc2n1COCC[Si](C)(C)C ; CO[C@H](C)c1nc2ccc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)cc2n1COCC[Si](C)(C)C]

Int. 2C32/2C33 3-((3-fluoro-4-(4-((1-(4-(1-((S)-1-(4-(2-((R)-1-methoxyethyl)-1-((2-(trimethylsilyl)-ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperazin-1-yl)phenyl)-amino)piperidine-2,6-dione and 3-((3-fluoro-4-(4-((1-(4-(1-((S)-1-(4-(2-((R)-1-methoxy-ethyl)-1-((2-(trimethylsilyl) ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)-piperidine-2,6-dione (0.15 g) prepared similarly to Int. 2C18 from Int. 2C34/2C35/2C15 (0.19 g/0.22 g, HCl salt).

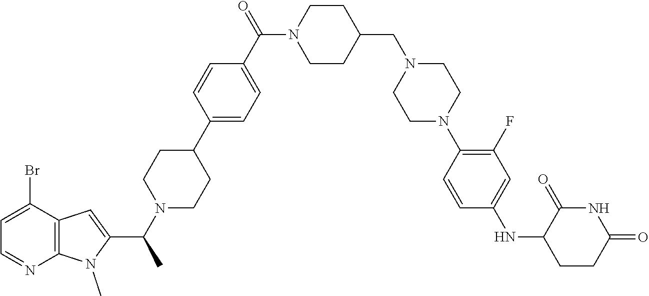 [SMILES: C[C@@H](c1cc2c(Br)ccnc2n1C)N1CCC(c2ccc(C(=O)N3CCC(CN4CCN(c5ccc(NC6CCC(=O)NC6=O)cc5F)CC4)CC3)cc2)CC1]

Int. 2C37. 3-((4-(4-((1-(4-(1-((S)-1-(4-bromo-1-methyl-1H-pyrrolo[2,3-b]pyridin-2-yl)ethyl)piperidin-4-yl)benzoyl)piperidin-4-yl)methyl)piperazin-1-yl)-3-fluorophenyl)-amino)piperidine-2,6-dione (2.5 g) was prepared similarly to Int. 2C18 from Ints. 2C36/2C15 (1.5 g/1.4 g, HCl salt).

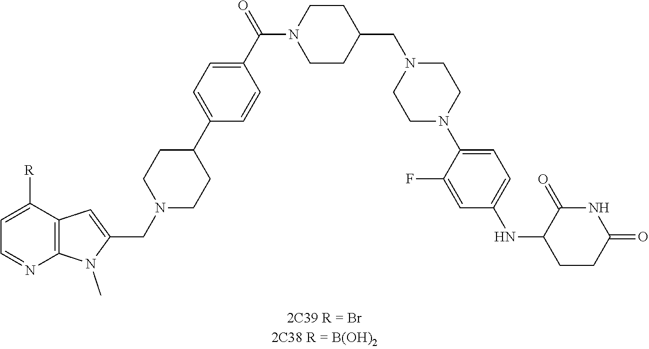

Int. 2C39 3-((4-(4-((1-(4-(1-((4-bromo-1-methyl-1H-pyrrolo[2,3-b]pyridin-2-yl)-methyl)piperidin-4-yl)benzoyl)-piperidin-4-yl)methyl)piperazin-1-yl)-3-fluorophenyl)-amino)piperidine-2,6-dione (0.17 g) was prepared similarly to Int. 2C18 using Ints. 1AC4/2C15 (0.15 g/0.17 g, HCl salt). Int. 2C39 (0.40 g), 4,4,4′, 4′, 5,5,5′, 5′-octamethyl-2,2′-bi (1,3,2-dioxaborolane) (0.23 g), KOAc (0.12 g), and PdDPPFCl₂-DCM (49 mg) were degassed in dioxane (5 mL) and stirred ON at 95-100° C. The residue after filtration and concentration was purified by FC (hexane/EtOAc 1:1) to give Int. 2C38 (2-((4-(4-(4-((4-(4-((2,6-dioxopiperidin-3-yl)amino)-2-fluorophenyl)-piperazin-1-yl)methyl)-piperidine-1-carbonyl)phenyl) piperidin-1-yl)methyl)-1-methyl-1H-pyrrolo[2,3-b]pyridin-4-yl) boronic acid (0.32 g).

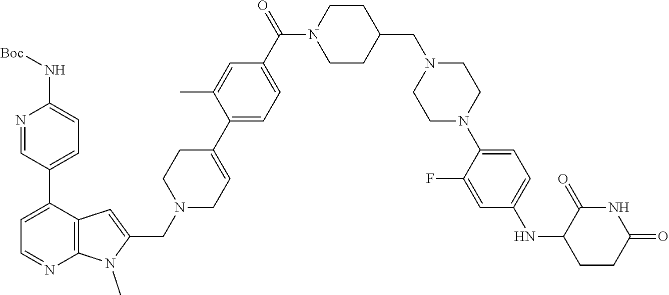 [SMILES: Cc1cc(C(=O)N2CCC(CN3CCN(c4ccc(NC5CCC(=O)NC5=O)cc4F)CC3)CC2)ccc1C1=CCN(Cc2cc3c(-c4ccc(NC(=O)OC(C)(C)C)nc4)ccnc3n2C)CC1]

Int. 2C45 tert-butyl (5-(2-((4-(4-(4-((4-(4-((2,6-dioxopiperidin-3-yl)amino)-2-fluorophenyl)-piperazin-1-yl)methyl)piperidine-1-carbonyl)-2-methylphenyl)-3,6-dihydropyridin-1(2H)-yl)methyl)-1-methyl-1H-pyrrolo[2,3-b]pyridin-4-yl)pyridin-2-yl) carbamate (27 mg) prepared similarly to Int. 2C18 from Ints. 2C40/2C15 (25 mg/20 mg, HCl salt).

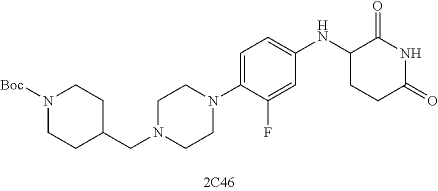 [SMILES: CC(C)(C)OC(=O)N1CCC(CN2CCN(c3ccc(NC4CCC(=O)NC4=O)cc3F)CC2)CC1]

Int. 2C48 (0.30 g), NaHCO₃ (0.27 g), and 3-bromo-piperidine-2,6-dione (0.29 g) were stirred ON in DMF (5 mL) at 80° C. The OL (EtOAc/water) was dried, concentrated, and purified by FC (pentane/EtOAc 1:0 to 1:1) to give Int. 2C46 tert-butyl 4-((4-(4-((2,6-dioxopiperidin-3-yl)amino)-2-fluorophenyl) piperazin-1-yl)-methyl)piperidine-1-carboxylate (0.19 g).

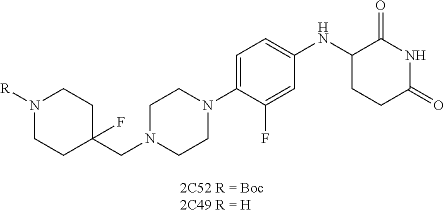

Int. 2C49 3-((3-fluoro-4-(4-((4-fluoropiperidin-4-yl)methyl)piperazin-1-yl)phenyl)amino)-piperidine-2,6-dione (0.17 g) was prepared from Int. 2C50 similarly to Int. 2N5.

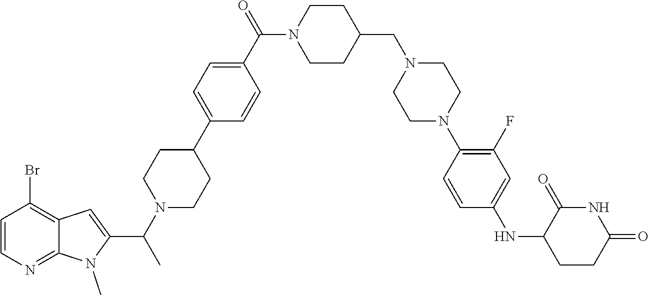 [SMILES: CC(c1cc2c(Br)ccnc2n1C)N1CCC(c2ccc(C(=O)N3CCC(CN4CCN(c5ccc(NC6CCC(=O)NC6=O)cc5F)CC4)CC3)cc2)CC1]

Int. 2C53. 3-((4-(4-((1-(4-(1-(1-(4-bromo-1-methyl-1H-pyrrolo[2,3-b]pyridin-2-yl)ethyl)piperidin-4-yl)-benzoyl)piperidin-4-yl)methyl)piperazin-1-yl)-3-fluorophenyl)amino)piperidine-2,6-dione (0.17 g) was prepared similarly to Int. 2C18 from Ints. 2C3/2C15 (0.35 g/0.27 g, HCl salt).

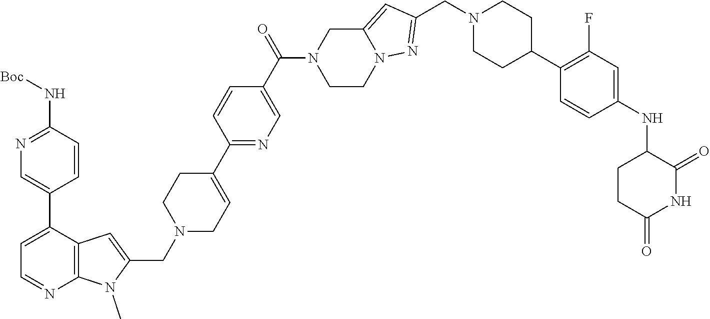 [SMILES: Cn1c(CN2CC=C(c3ccc(C(=O)N4CCn5nc(CN6CCC(c7ccc(NC8CCC(=O)NC8=O)cc7F)CC6)cc5C4)cn3)CC2)cc2c(-c3ccc(NC(=O)OC(C)(C)C)nc3)ccnc21]

Et₃N (50 uL) was added to a mixture of Ints. 2C66/2Q15 (20 mg/19 mg) and HATU (16 mg) in DMF (3 mL) and stirred ON and concentrated to give Int. 2C70 tert-butyl (5-(2-((5-(2-((4-(4-((2,6-dioxopiperidin-3-yl)amino)-2-fluorophenyl) piperidin-1-yl)methyl)-4,5,6,7-tetrahydropyrazolo [1,5-a]pyrazine-5-carbonyl)-3′, 6′-dihydro-[2,4′-bipyridin]-1′ (2′H)-yl)methyl)-1-methyl-1H-pyrrolo [2,3-b] pyridin-4-yl)pyridin-2-yl)-carbamate (34 mg).

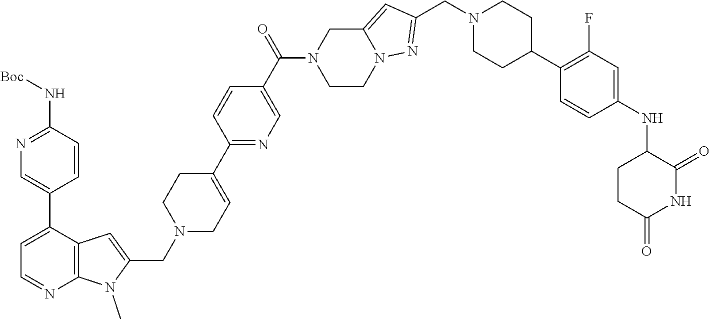 [SMILES: Cn1c(CN2CC=C(c3ccc(C(=O)N4CCn5nc(CN6CCC(c7ccc(NC8CCC(=O)NC8=O)cc7F)CC6)cc5C4)cn3)CC2)cc2c(-c3ccc(NC(=O)OC(C)(C)C)nc3)ccnc21]

Et₃N (15 uL) was added to a mixture of Ints. 2C60/2Q15 (19 mg/20 mg) and HATU (16 mg) in DMF (2 mL), stirred ON, and concentrated to give Int. 2C71 tert-butyl (6-(2-((5-(2-((4-(4-((2,6-dioxopiperidin-3-yl)amino)-2-fluorophenyl) piperidin-1-yl)methyl)-4,5,6,7-tetrahydropyrazolo[1,5-a]pyrazine-5-carbonyl)-3′, 6′-dihydro-[2,4′-bipyridin]-1′ (2′H)-yl)methyl)-1-methyl-1H-pyrrolo [2,3-b] pyridin-4-yl)pyridazin-3-yl) carbamate (34 mg).

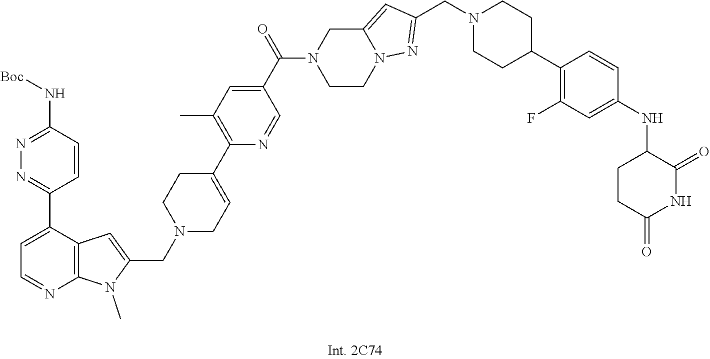 [SMILES: Cc1cc(C(=O)N2CCn3nc(CN4CCC(c5ccc(NC6CCC(=O)NC6=O)cc5F)CC4)cc3C2)cnc1C1=CCN(Cc2cc3c(-c4ccc(NC(=O)OC(C)(C)C)nn4)ccnc3n2C)CC1]

Et₃N (14 uL) was added to a mixture of Ints. 2C75/2Q15 (19 mg/19 mg) and HATU (16 mg) in DMF (2 mL) and stirred ON at RT and concentrated to give Int. 2C74 (34 mg).

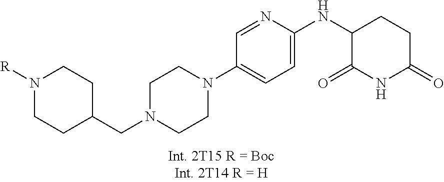

Int. 2T16 (40 mg), 3-bromo-piperidine-2,6-dione (41 mg), and NaHCO₃ (36 mg) were stirred 40 h in DMF (2 mL) at 80° C. These two reactions were combined and HPLC-purified to give Int. 2T15 tert-butyl 4-((4-(6-((2,6-dioxopiperidin-3-yl)amino)pyridin-3-yl) piperazin-1-yl)methyl)-piperidine-1-carboxylate (26 mg). Int. 2T15 (24 mg) was stirred 1 h in DCM/TFA (1:0.7, 1.7 mL) and concentrated to give Int. 2T14 3-((5-(4-(piperidin-4-yl-methyl)piperazin-1-yl)pyridin-2-yl)amino)piperidine-2,6-dion (19 mg, TFA salt).

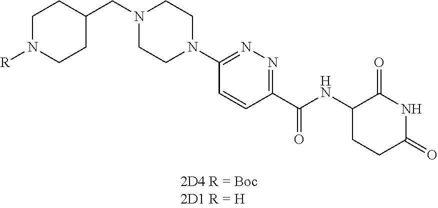

Int. 2D3 (0.6 g), 3-aminopiperidine-2,6-dione (0.19 g), and HATU (0.84 g) were stirred 3 h in DMF/DIPEA (7.7:1, 11.3 mL). The OL (EtOAc/water) was washed with brine, dried, concentrated, and triturated in pentane to give Int. 2D4 tert-butyl 4-((4-(6-((2,6-dioxopiperidin-3-yl) carbamoyl)-pyridazin-3-yl) piperazin-1-yl)methyl)piperidine-1-carboxylate (0.6 g). This material was stirred ON in 2M HCl in 1,4-dioxane (20 mL) at 10° C. to RT and concentrated. The was triturated in Et₂O to give Int. 2D1 N-(2,6-dioxopiperidin-3-yl)-6-(4-(piperidin-4-ylmethyl)piperazin-1-yl) pyridazine-3-carboxamide (0.35 g, HCl salt).

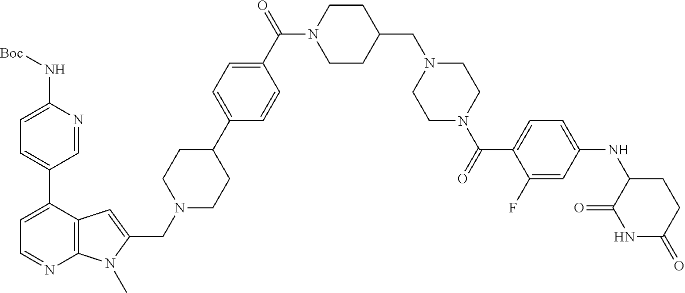 [SMILES: Cn1c(CN2CCC(c3ccc(C(=O)N4CCC(CN5CCN(C(=O)c6ccc(NC7CCC(=O)NC7=O)cc6F)CC5)CC4)cc3)CC2)cc2c(-c3ccc(NC(=O)OC(C)(C)C)nc3)ccnc21]

Int. 2E1 tert-butyl (5-(2-((4-(4-(4-((4-(4-((2,6-dioxopiperidin-3-yl)amino)-2-fluorobenzoyl) piperazin-1-yl)methyl)piperidine-1-carbonyl)phenyl) piperidin-1-yl)methyl)-1-methyl-1H-pyrrolo[2,3-b]pyridin-4-yl)pyridin-2-yl) carbamate (33 mg) prepared similarly to Int. 2C18 from Ints. 2E6/2E2 (25 mg/32 mg, TFA salt).

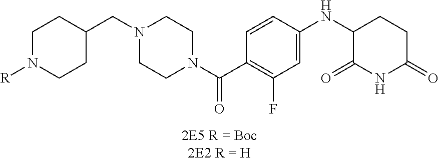

Int. 2E4 (0.18 g), 3-bromo-piperidine-2,6-dione (0.12 g), and NaHCO₃ (0.14 g) were stirred ON in DMF (5 mL) at 70° C. 3-Bromo-piperidine-2,6-dione (0.12 g) and NaHCO₃ (0.14 g) were added and stirring continued 6 h at 70° C. 3-Bromo-piperidine-2,6-dione (0.24 g) was added and stirring continued 48 h at 70° C. The OL (EtOAc/water) was concentrated and purified by FC (heptane/EtOAc/MeOH 1:0:0 to 0:95:5) to give Int. 2E5 tert-butyl 4-((4-(4-((2,6-dioxo-piperidin-3-yl)-amino)-2-fluorobenzoyl)-piperazin-1-yl)methyl)piperidine-1-carboxylate (41 mg). This material was stirred 0.75 h in DCM/TFA (1:1, 30 mL) and concentrated to give Int. 2E2 3-((3-fluoro-4-(4-(piperidin-4-ylmethyl)piperazine-1-carbonyl)phenyl)amino)-piperidine-2,6-dione (60 mg, TFA salt).

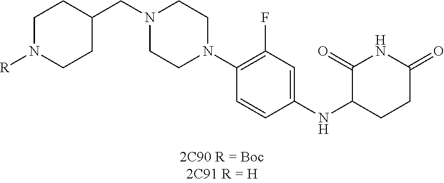

Int. 2C89 (3.0 g), 3-bromo-piperidine-2,6-dione (4.4 g), and NaHCO₃ (3.0 g) in DMF (60 mL) were stirred 48 h. The OL (EtOAc/water) was dried, concentrated, and purified by FC (hexane/EtOAc 3:7) to give Int. 2C90 tert-butyl 4-((4-(4-((2,6-dioxopiperidin-3-yl)amino)-2-fluoro-phenyl)-piperazin-1-yl)methyl)piperidine-1-carboxylate (2.1 g), which was stirred 3 h in DCM/4M HCl in dioxane (2:1, 30 mL). The mixture was concentrated and triturated in Et₂O/MTBE/MeOH to give Int. 2C91 3-((3-fluoro-4-(4-(piperidin-4-ylmethyl)-piperazin-1-yl)phenyl)amino)piperidine-2,6-dione (1.4 g, HCl salt).

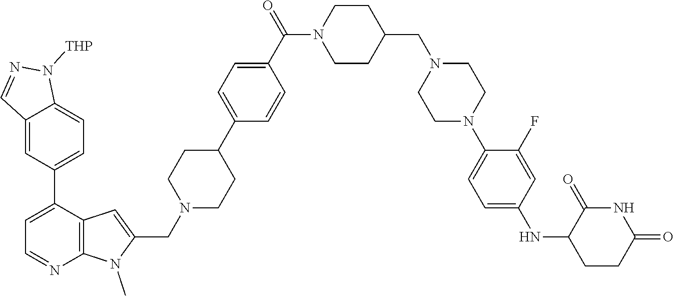

HCl salts of Ints. 3A2/2C91 (0.31 g/0.25 g) and HATU (0.22 g) were stirred ON in DMF/DIPEA (15:1, 6.4 mL) at 0° C. to RT. The mixture was diluted with water to precipitate a solid that was purified by FC (DCM/MeOH 9:1) to give Int. 2C92 3-((3-fluoro-4-(4-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)piperazin-1-yl)phenyl)amino)piperidine-2,6-dione (0.35 g).

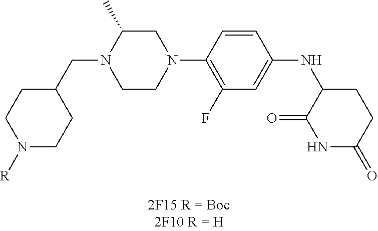

Int. 2F14 (1.0 g), NaHCO₃ (0.3 g), and 3-bromo-piperidine-2,6-dione (0.7 g) were stirred ON in DMF (20 mL) at 0-80° C. The OL (EtOAc/water) was concentrated and purified by FC (pentane/EtOAc 1:0 to 9:1) to give Int. 2F15 tert-butyl 4-(((2R)-4-(4-((2,6-dioxopiperidin-3-yl)amino)-2-fluorophenyl)-2-methylpiperazin-1-yl)-methyl)piperidine-1-carboxylate (0.8 g). Int. 2F15 (0.25 g) was stirred 6 h in 0.7 M HCl in dioxane (12 mL) and concentrated to give Int. 2F10 3-((3-fluoro-4-((R)-3-methyl-4-(piperidin-4-ylmethyl)piperazin-1-yl)phenyl)amino)-piperidine-2,6-dione (0.20 g, HCl salt).

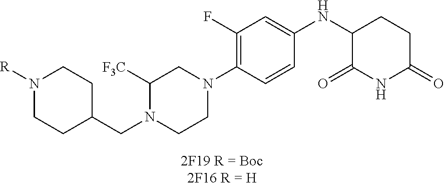

Int. 2F18 (0.55 g), NaHCO₃ (0.27 g), and 3-bromopiperidine-2,6-dione (0.21 g) were stirred ON in DMF (5 mL) at 80° C. The OL (EtOAc/water) was concentrated and purified by FC (EtOAc/pentane 7:3 to 4:1) to give Int. 2F19 tert-butyl 4-((4-(4-((2,6-dioxopiperidin-3-yl)amino)-2-fluorophenyl)-2-(trifluoromethyl)-piperazin-1-yl)methyl)piperidine-1-carboxylate (0.33 g). This material was stirred 2 h in DCM/4M HCl in dioxane (2.7:1, 11 mL) and concentrated to give Int. 2F16 3-((3-fluoro-4-(4-(piperidin-4-ylmethyl)-3-(trifluoromethyl)piperazin-1-yl)phenyl)amino)piperidine-2,6-dione (0.25 g, HCl salt).

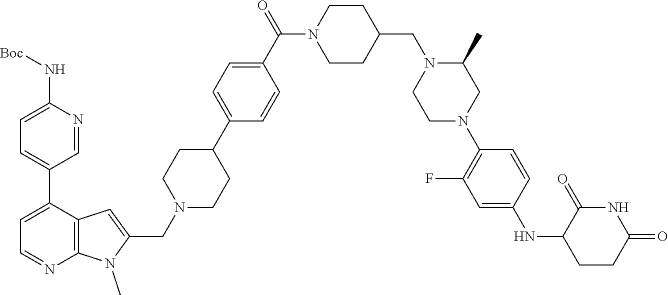 [SMILES: C[C@H]1CN(c2ccc(NC3CCC(=O)NC3=O)cc2F)CCN1CC1CCN(C(=O)c2ccc(C3CCN(Cc4cc5c(-c6ccc(NC(=O)OC(C)(C)C)nc6)ccnc5n4C)CC3)cc2)CC1]

Int. 2F20 tert-butyl (5-(2-((4-(4-(4-(((2S)-4-(4-((2,6-dioxopiperidin-3-yl)amino)-2-fluorophenyl)-2-methylpiperazin-1-yl)methyl)piperidine-1-carbonyl)phenyl) piperidin-1-yl)methyl)-1-methyl-1H-pyrrolo[2,3-b]pyridin-4-yl)pyridin-2-yl) carbamate (50 mg) prepared similarly to Int. 2C18 from HCl salts of Ints. 2E6/2F21 (48 mg/34 mg).

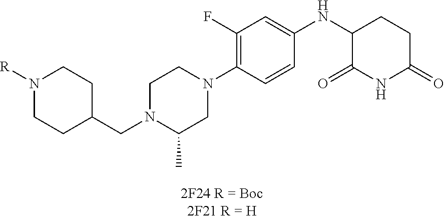

Int. 2F21 3-((3-fluoro-4-((S)-3-methyl-4-(piperidin-4-ylmethyl)piperazin-1-yl)phenyl)amino)piperidine-2,6-dione (82 mg, HCl salt) prepared similarly to Int. 2F10 from Int. 2F22 (0.74 g).

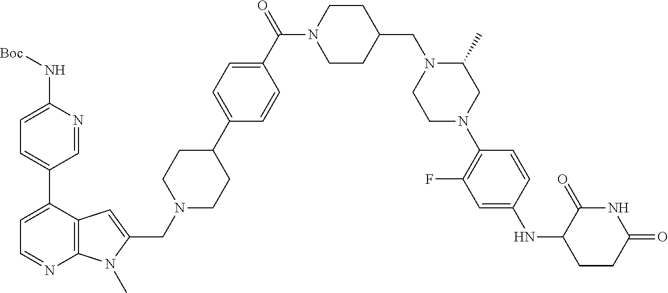 [SMILES: C[C@@H]1CN(c2ccc(NC3CCC(=O)NC3=O)cc2F)CCN1CC1CCN(C(=O)c2ccc(C3CCN(Cc4cc5c(-c6ccc(NC(=O)OC(C)(C)C)nc6)ccnc5n4C)CC3)cc2)CC1]

Int. 2F27 tert-butyl (5-(2-((4-(4-(4-(((2R)-4-(4-((2,6-dioxopiperidin-3-yl)amino)-2-fluorophenyl)-2-methylpiperazin-1-yl)methyl)piperidine-1-carbonyl)phenyl) piperidin-1-yl)methyl)-1-methyl-1H-pyrrolo[2,3-b]pyridin-4-yl)pyridin-2-yl) carbamate (39 mg) prepared similarly to Int. 2C18 from Ints. 2E6/2F21 (34 mg/HCl salt).

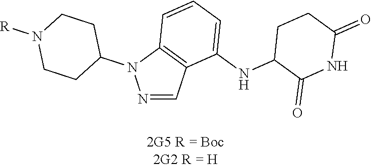

Int. 2G4 (63 mg), NaHCO₃ (67 mg), and 3-bromo-piperidine-2,6-dione (57 mg) were stirred ON in DMF (2 mL) at 70° C. The OL (EtOAc/water) was concentrated and purified by FC (heptane/EtOAc 4:1 to 0:1) to give Int. 2G5 tert-butyl 4-[4-[(2,6-dioxo-3-piperidyl)amino]-indazol-1-yl]piperidine-1-carboxylate (62 mg). This material was stirred 0.5 h in DCM/TFA (10:1, 2.2 mL) and concentrated to give Int. 2G2 3-((1-(piperidin-4-yl)-1H-indazol-4-yl)amino)piperidine-2,6-dione (64 mg, TFA salt).

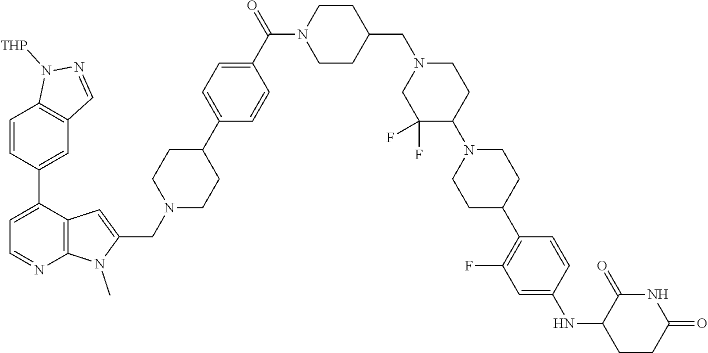

Int. 2H1 3-((4-(3′, 3′-difluoro-1′-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)-[1,4′-bipiperidin]-4-yl)-3-fluorophenyl)amino)piperidine-2,6-dione (22 mg) prepared similarly to Int. 2C18 from HCl salts of Ints. 3A2/2H2 (24 mg/22 mg).

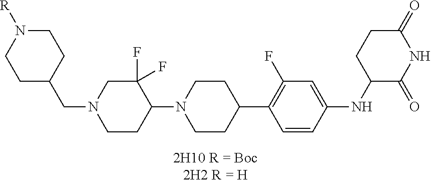

Int. 2H9 (0.8 g), 3-bromo-piperidine-2,6-dione (0.36 g), and NaHCO₃ (0.4 g) were stirred ON in DMF (6 mL) at 80° C. 3-Bromo-piperidine-2,6-dione (0.30 g) and NaHCO₃ (0.2 g) were added and stirring continued ON at 80° C. The OL (EtOAc/water) was concentrated and purified by FC (EtOAc/pentane 7:3 to 4:1) to give Int. 2H10 tert-butyl 4-((4-(4-((2,6-dioxopiperidin-3-yl)amino)-2-fluoro-phenyl)-3′, 3′-difluoro-[1,4′-bipiperidin]-1′-yl)-methyl)-piperidine-1-carboxylate (0.25 g). A solution of Int. 2H10 (50 mg) in 1.3M HCl in dioxane (1.5 mL) was stirred 2 h at 0° C. to RT, concentrated, and triturated in Et₂O Int. 2H2 3-((4-(3′, 3′-difluoro-1′-(piperidin-4-ylmethyl)-[1,4′-bipiperidin]-4-yl)-3-fluorophenyl)-amino)piperidine-2,6-dione (30 mg, HCl salt).

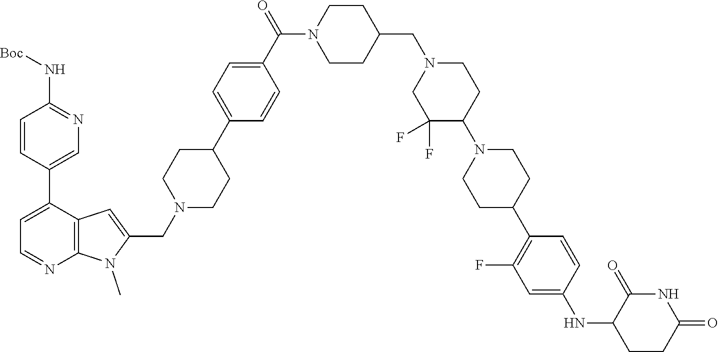 [SMILES: Cn1c(CN2CCC(c3ccc(C(=O)N4CCC(CN5CCC(N6CCC(c7ccc(NC8CCC(=O)NC8=O)cc7F)CC6)C(F)(F)C5)CC4)cc3)CC2)cc2c(-c3ccc(NC(=O)OC(C)(C)C)nc3)ccnc21]

Int. 2H11 tert-butyl (5-(2-((4-(4-(4-((4-(4-((2,6-dioxopiperidin-3-yl)amino)-2-fluorophenyl)-3′, 3′-difluoro-[1,4′-bipiperidin]-1′-yl)methyl)piperidine-1-carbonyl)phenyl) piperidin-1-yl)methyl)-1-methyl-1H-pyrrolo[2,3-b]pyridin-4-yl)pyridin-2-yl) carbamate (33 mg) prepared similarly to Int. 2C18 from HCl salts of Ints. 2E6/2H2 (20 mg/24 mg).

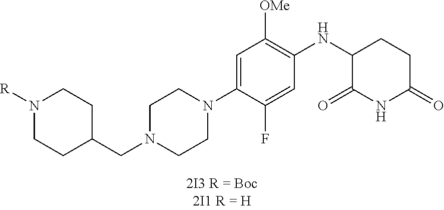

Int. 2I1 3-((5-fluoro-2-methoxy-4-(4-(piperidin-4-ylmethyl)piperazin-1-yl)phenyl)amino)piperidine-2,6-dione (0.90 g, HCl salt) prepared similarly to Int. 2E2 from Int. 2I4 (4 g).

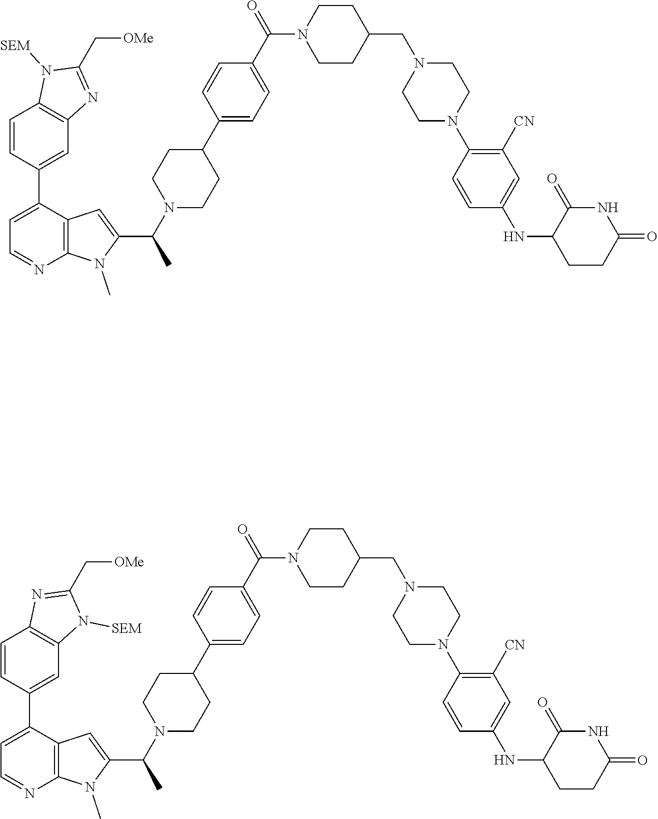 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8C#N)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ccc(NC9CCC(=O)NC9=O)cc8C#N)CC7)CC6)cc5)CC3)n4C)cc2n1COCC[Si](C)(C)C]

Int. 215/216 5-((2,6-dioxopiperidin-3-yl)amino)-2-(4-((1-(4-(1-((S)-1-(4-(2-(methoxy-methyl)-1-((2-(trimethyl-silyl) ethoxy)methyl)-1H-benzo[d]imidazol-5-yl)-1-methyl-1H-pyrrolo[2,3-b]pyridin-2-yl)ethyl)-piperidin-4-yl)-benzoyl)piperidin-4-yl)methyl)piperazin-1-yl)benzonitrile and 5-((2,6-dioxopiperidin-3-yl)amino)-2-(4-((1-(4-(1-((S)-1-(4-(2-(methoxymethyl)-1-((2-(trimethylsilyl) ethoxy)methyl)-1H-benzo[d]-imidazol-6-yl)-1-methyl-1H-pyrrolo[2,3-b]pyridin-2-yl)ethyl)piperidin-4-yl)benzoyl)piperidin-4-yl)-methyl)piperazin-1-yl)-benzonitrile (0.12 g) prepared similarly to Int. 2C18 from Ints. 2F8/2F9/2I13 (0.10 g/96 mg, HCl salt).

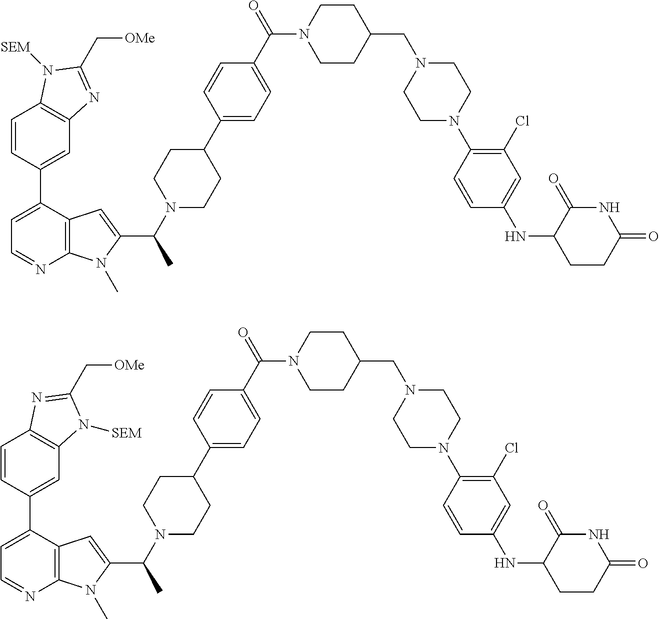 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8Cl)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ccc(NC9CCC(=O)NC9=O)cc8Cl)CC7)CC6)cc5)CC3)n4C)cc2n1COCC[Si](C)(C)C]

Int. 2I7/2I8 3-((3-chloro-4-(4-((1-(4-(1-((S)-1-(4-(2-(methoxymethyl)-1-((2-(trimethylsilyl)-ethoxy)-methyl)-1H-benzo[d]imidazol-5-yl)-1-methyl-1H-pyrrolo[2,3-b]pyridin-2-yl)ethyl)-piperidin-4-yl)benzoyl)-piperidin-4-yl)-methyl)piperazin-1-yl)phenyl)amino)piperidine-2,6-dione and 3-((3-chloro-4-(4-((1-(4-(1-((S)-1-(4-(2-(methoxy-methyl)-1-((2-(trimethylsilyl)-ethoxy)methyl)-1H-benzo[d] imidazol-6-yl)-1-methyl-1H-pyrrolo[2,3-b]pyridin-2-yl)ethyl)-piperidin-4-yl)benzoyl)piperidin-4-yl)methyl)piperazin-1-yl)phenyl)amino)piperidine-2,6-dione (0.17 g) prepared similarly to Int. 2C18 from Ints. 2F8/2F9/2I9 (0.20 g/0.21 g, HCl salt).

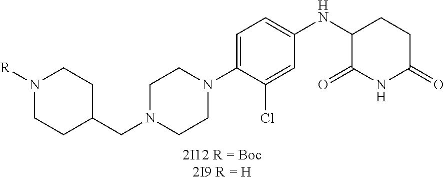

Int. 2I9 3-((3-chloro-4-(4-(piperidin-4-ylmethyl)piperazin-1-yl)-phenyl)amino)-piperidine-2,6-dione (0.60 g, HCl salt) prepared similarly to Int. 2E2 from Int. 2I10 (2 g).

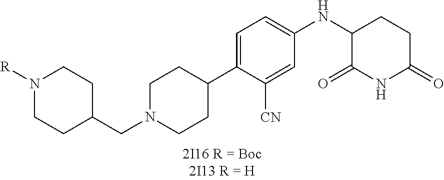

Int. 2I13 5-((2,6-dioxopiperidin-3-yl)amino)-2-(4-(piperidin-4-ylmethyl)piperazin-1-yl)-benzonitrile (0.23 g, HCl salt) prepared similarly to Int. 2E2 from Int. 2I4 (2 g).

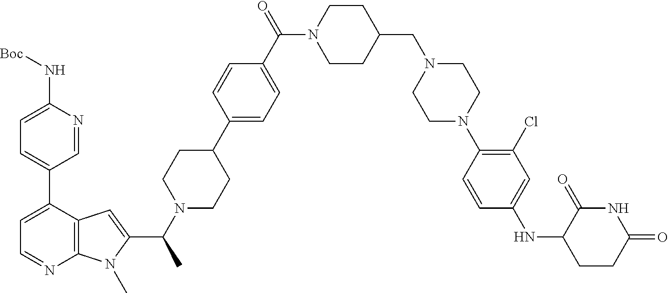 [SMILES: C[C@@H](c1cc2c(-c3ccc(NC(=O)OC(C)(C)C)nc3)ccnc2n1C)N1CCC(c2ccc(C(=O)N3CCC(CN4CCN(c5ccc(NC6CCC(=O)NC6=O)cc5Cl)CC4)CC3)cc2)CC1]

Int. 2I17 tert-butyl (5-(2-((1S)-1-(4-(4-(4-((4-(2-cyano-4-((2,6-dioxopiperidin-3-yl)amino)-phenyl)-piperazin-1-yl)methyl)piperidine-1-carbonyl)phenyl) piperidin-1-yl)ethyl)-1-methyl-1H-pyrrolo[2,3-b]pyridin-4-yl)pyridin-2-yl) carbamate (0.15 g) prepared similarly to Int. 2C18 from Ints. 3E76/2I13 (0.10 g/81 mg, HCl salt).

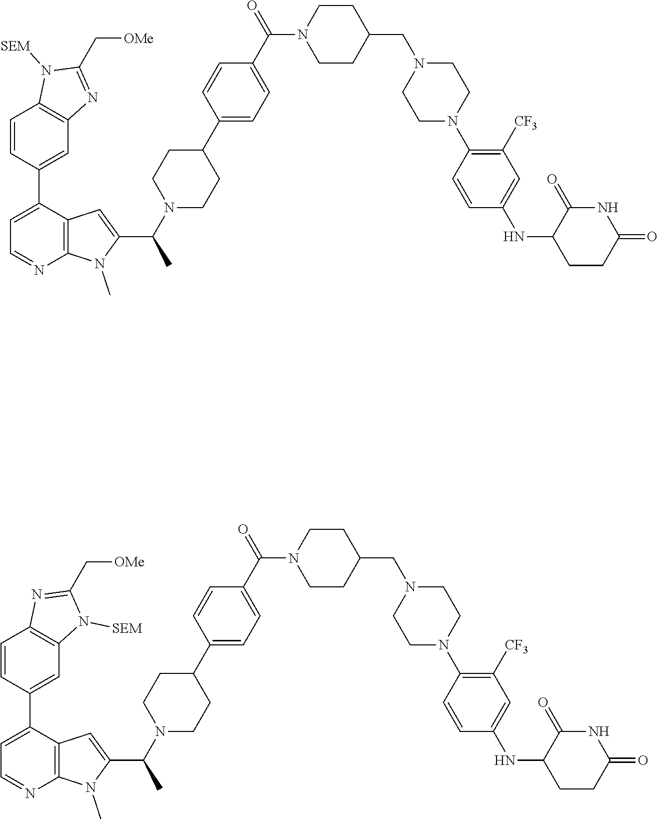 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8C(F)(F)F)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ccc(NC9CCC(=O)NC9=O)cc8C(F)(F)F)CC7)CC6)cc5)CC3)n4C)cc2n1COCC[Si](C)(C)C]

Int. 2I8/2I19 3-((4-(4-((1-(4-(1-((S)-1-(4-(2-(methoxymethyl)-1-((2-(trimethylsilyl)-ethoxy)methyl)-1H-benzo[d]imidazol-5-yl)-1-methyl-1H-pyrrolo[2,3-b]pyridin-2-yl)ethyl)-piperidin-4-yl)benzoyl)piperidin-4-yl)-methyl)piperazin-1-yl)-3-(trifluoromethyl)phenyl)-amino)piperidine-2,6-dione and 3-((4-(4-((1-(4-(1-((S)-1-(4-(2-(methoxymethyl)-1-((2-(trimethylsilyl) ethoxy)methyl)-1H-benzo[d] imidazol-6-yl)-1-methyl-1H-pyrrolo[2,3-b]pyridin-2-yl)ethyl)piperidin-4-yl)benzoyl)piperidin-4-yl)methyl)piperazin-1-yl)-3-(trifluoromethyl)-phenyl)-amino)piperidine-2,6-dione (0.50 g) prepared similarly to Int. 2C18 from Ints. 2F8/2F9/2I2-0 (0.20 g/0.58 g, HCl salt).

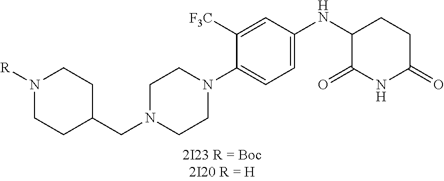

Int. 2I20 3-((4-(4-(piperidin-4-ylmethyl)-piperazin-1-yl)-3-(trifluoromethyl)phenyl)amino)-piperidine-2,6-dione (1.0 g, HCl salt) was prepared from Int. 2I21 (4 g) similarly to Int. 2E2.

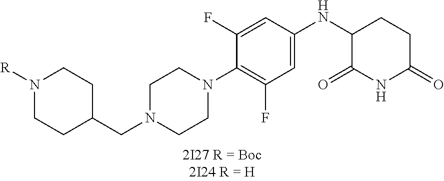

Int. 2I24 3-((3,5-difluoro-4-(4-(piperidin-4-ylmethyl)piperazin-1-yl)phenyl)-amino)piperidine-2,6-dione (0.14 g, HCl salt) prepared similarly to Int. 2E2 from Int. 2I25. Int. 2I25 (0.45 g) prepared similarly to Int. 2I4 from 1,2,3-trifluoro-5-nitrobenzene.

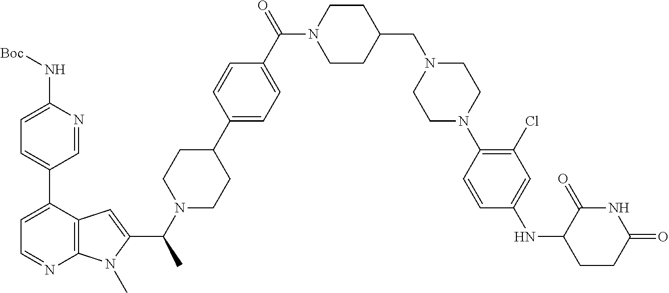 [SMILES: C[C@@H](c1cc2c(-c3ccc(NC(=O)OC(C)(C)C)nc3)ccnc2n1C)N1CCC(c2ccc(C(=O)N3CCC(CN4CCN(c5ccc(NC6CCC(=O)NC6=O)cc5Cl)CC4)CC3)cc2)CC1]

Int. 2I28 tert-butyl (5-(2-((1S)-1-(4-(4-(4-((4-(2-chloro-4-((2,6-dioxopiperidin-3-yl)amino)-phenyl) piperazin-1-yl)methyl)piperidine-1-carbonyl)phenyl) piperidin-1-yl)ethyl)-1-methyl-1H-pyrrolo[2,3-b]pyridin-4-yl)pyridin-2-yl) carbamate (60 mg) prepared similarly to Int. 2C18 from Ints. 3E76/2I9 (0.10 g/0.16 g, HCl salt).

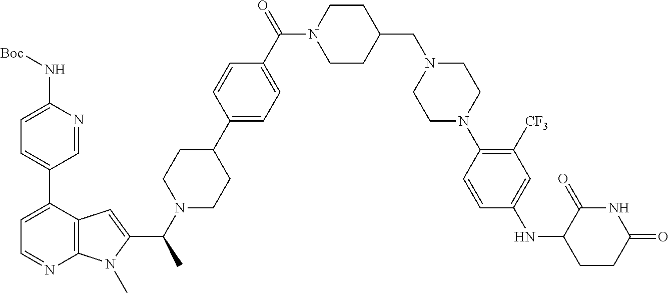 [SMILES: C[C@@H](c1cc2c(-c3ccc(NC(=O)OC(C)(C)C)nc3)ccnc2n1C)N1CCC(c2ccc(C(=O)N3CCC(CN4CCN(c5ccc(NC6CCC(=O)NC6=O)cc5C(F)(F)F)CC4)CC3)cc2)CC1]

Int. 2I29 tert-butyl (5-(2-((1S)-1-(4-(4-(4-((4-(4-((2,6-dioxopiperidin-3-yl)amino)-2-(trifluoromethyl)phenyl)-piperazin-1-yl)methyl)piperidine-1-carbonyl)phenyl) piperidin-1-yl)ethyl)-1-methyl-1H-pyrrolo[2,3-b]pyridin-4-yl)pyridin-2-yl) carbamate (0.25 g) prepared similarly to Int. 2C18 from Ints. 3E76/2I20 (0.15 g/0.51 g, HCl salt).

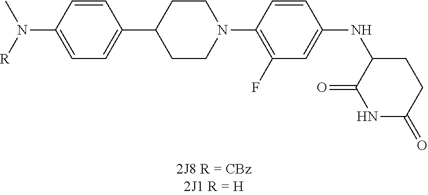

Int. 2J7 (0.25 g), NaHCO₃ (0.19 g), and 3-bromo-piperidine-2,6-dione (0.17 g) were stirred 2 h in DMF (5 mL) at 70° C. NaHCO₃ (0.19 g) and 3-bromo-piperidine-2,6-dione (0.11 g) were added and stirring continued ON at 70° C. and concentrated. The OL (EtOAc/water) was washed with brine, dried, concentrated, and purified by FC (heptane/EtOAc 1:0 to 2:3) to give Int. 2J8 benzyl (4-(1-(4-((2,6-dioxopiperidin-3-yl)amino)-2-fluorophenyl)-piperidin-4-yl)phenyl) (methyl) carbamate (0.17 g). This material was hydrogenated 4 h using 10% Pd/C (25 mg) in THF/MeOH (3:1, 20 mL), filtered, and concentrated to give Int. 2J1 3-((3-fluoro-4-(4-(4-(methylamino)phenyl) piperidin-1-yl)phenyl)amino)piperidine-2,6-dione (0.13 g).

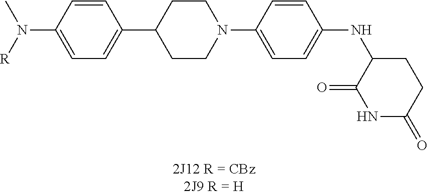

Int. 2J9. 3-((4-(4-(4-(methylamino)phenyl) piperidin-1-yl)phenyl)amino)piperidine-2,6-dione (0.11 g) was prepared similarly to Int. 2J11 from Int. 2J9.

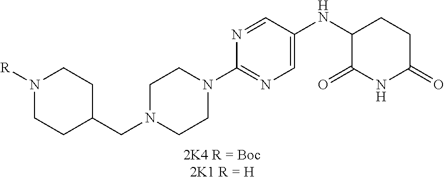

Int. 2K1 3-((2-(4-(piperidin-4-ylmethyl)piperazin-1-yl)pyrimidin-5-yl)amino)-piperidine-2,6-dione (32 mg, HCl salt) prepared similarly to Int. 2E2 from Int. 2K2 (0.19 g).

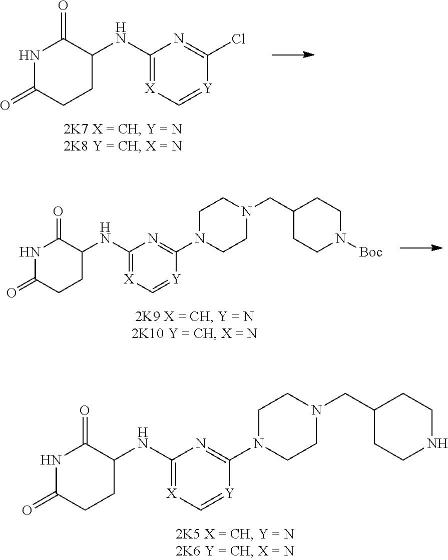

2,4-Dichloropyrimidine (91 mg) and 3-aminopiperidine-2,6-dione (0.10 g) were stirred 3 h at 120° C. and 48 h at RT in IPA/DIPEA (7.5:1, 3.4 mL). Another reaction was carried out in a similarly using NMP instead of IPA. 2/3 of the combined mixtures were reacted with tert-butyl 4-(piperazin-1-ylmethyl)-piperidine-1-carboxylate (80 mg) in the presence of DIPEA (0.1 mL) for 48 h at 100° C. tert-Butyl 4-(piperazin-1-ylmethyl)-piperidine-1-carboxylate (0.10 g) was added and stirring continued ON at 115° C. The mixture was HPLC-purified to give Int. 2K9 tert-butyl 4-((4-(4-((2,6-dioxo-piperidin-3-yl)amino)-pyrimidin-2-yl)-piperazin-1-yl)-methyl)piperidine-1-carboxylate (31 mg) and Int. 2K10 tert-butyl 4-((4-(2-((2,6-dioxopiperidin-3-yl)amino)-pyrimidin-4-yl) piperazin-1-yl)-methyl)-piperidine-1-carboxylate (15 mg). Int. 2K9 (25 mg) was stirred 1 h in DCM/TFA (20:1, 21 mL) and concentrated to give Int. 2K5 3-((2-(4-(piperidin-4-ylmethyl)-piperazin-1-yl)-pyrimidin-4-yl)amino)-piperidine-2,6-dione (19 mg, TFA salt). Int. 2K6 3-((2-(4-(piperidin-4-ylmethyl)-piperazin-1-yl)pyrimidin-4-yl)amino)-piperidine-2,6-dione was prepared similarly from Int. 2K10.

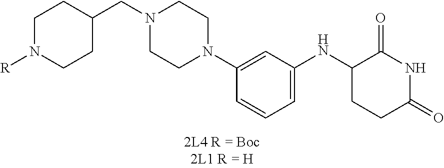

Int. 2L3 (0.19 g), 3-bromo-piperidine-2,6-dione (0.20 g), and NaHCO₃ (0.11 g) were stirred 2 h in DMF (5 mL) at 90° C. and HPLC-purified to give Int. 2L4 tert-butyl 4-((4-(3-((2,6-dioxo-piperidin-3-yl)-amino)-phenyl)-piperazin-1-yl)methyl)piperidine-1-carboxylate (92 mg). Int. 2L4 (50 mg) was stirred 1 h in DCM/TFA (1:1, 2 mL) and concentrated to give Int. 2L1 3-((3-(4-(piperidin-4-ylmethyl)piperazin-1-yl)phenyl)amino)-piperidine-2,6-dione (40 mg, TFA salt).

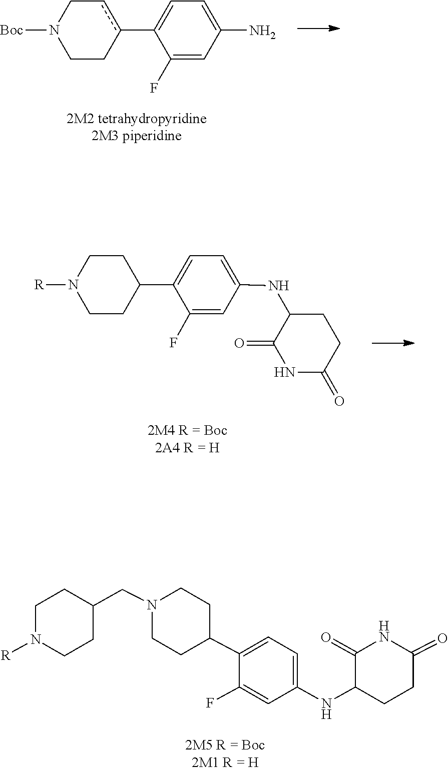

3-Bromopiperidine-2,6-dione (0.27 g), Int. 2M3 (0.27 g), and NaHCO; (0.39 g) were stirred ON in DMF (4 mL) at 80° C. 3-Bromopiperidine-2,6-dione (0.10 g) and NaHCO₃ (0.12 g) were added and stirring continued ON at 71° C. The OL (EtOAc/water) was washed with brine, dried, concentrated, and purified by FC (heptane/EtOAc 1:0 to 2:3) to give Int. 2M4 tert-butyl 4-(4-((2,6-dioxopiperidin-3-yl)amino)-2-fluorophenyl)-piperidine-1-carboxylate (0.15 g). This material was stirred 2 h in DCM/TFA (1:1, 12 mL) at 0° C. and concentrated to give Int. 2A4 3-((3-fluoro-4-(piperidin-4-yl)-phenyl)amino)-piperidine-2,6-dione (0.17 g, TFA salt). This material, tert-butyl 4-formyl-piperidine-1-carbo-xylate (0.15 g), and STAB (0.34 g) were stirred 1.5 h in DMF (3 mL) and HPLC-purified to give Int. 2M5 tert-butyl 4-((4-(4-((2,6-dioxo-piperidin-3-yl)amino)-2-fluorophenyl) piperidin-1-yl)methyl)-piperidine-1-carboxylate (0.13 g). This material was stirred 1 h in DCM/TFA (1:1, 6 mL) and concentrated to give Int. 2M1 3-((3-fluoro-4-(1-(piperidin-4-ylmethyl)piperidin-4-yl)phenyl)amino)piperidine-2,6-dione (0.19 g, TFA salt).

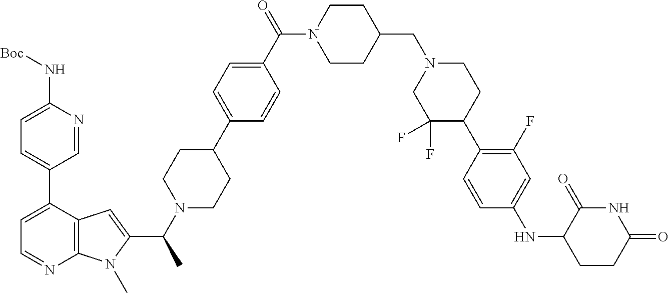 [SMILES: C[C@@H](c1cc2c(-c3ccc(NC(=O)OC(C)(C)C)nc3)ccnc2n1C)N1CCC(c2ccc(C(=O)N3CCC(CN4CCC(c5ccc(NC6CCC(=O)NC6=O)cc5F)C(F)(F)C4)CC3)cc2)CC1]

Int. 2M6 tert-butyl (5-(2-((1S)-1-(4-(4-(4-((4-(4-((2,6-dioxopiperidin-3-yl)amino)-2-fluorophenyl)-3,3-difluoropiperidin-1-yl)methyl)piperidine-1-carbonyl)phenyl) piperidin-1-yl)ethyl)-1-methyl-1H-pyrrolo[2,3-b]pyridin-4-yl)pyridin-2-yl) carbamate (0.11 g) prepared similarly to Int. 2C18 from Ints. 3E76/2M7 (85 mg/0.12 g, HCl salt).

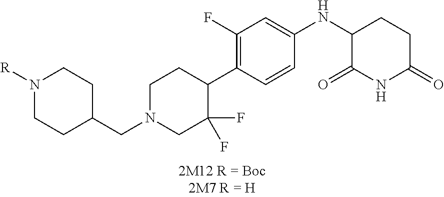

Int. 2M9 (0.73 g) and tert-butyl 4-formylpiperidine-1-carboxylate (0.39 g) were stirred 3 h in DCE/DIPEA (10:1, 19.6 mL) at 0° C. to RT. STAB (0.78 g) was added and stirring continued ON before concentration. The OL (water/MeOH/DCM (1:9)) was washed with brine, dried, concentrated, and purified by FC (hexane/EtOAc 3:2 to 1:1) to give Int. 2M10 tert-butyl 4-((4-(4-(((benzyloxy)-carbonyl)amino)-2-fluorophenyl)-3,3-difluoro-3,6-dihydropyridin-1(2H)-yl)methyl)-piperidine-1-carboxylate (0.70 g). This material was hydrogenated 48 h using 10% Pd/C (0.25 g) in EtOAc (15 mL), filtered, concentrated, and purified by FC (hexane/EtAOC 1:4 to 0:1) to give Int. 2M11 tert-butyl 4-((4-(4-amino-2-fluorophenyl)-3,3-difluoropiperidin-1-yl)methyl)piperidine-1-carboxylate (0.21 g). Int. 2M11 (0.30 g), 3-bromo-piperidine-2,6-dione (0.54 g), and NaHCO₃ (0.18 g) were stirred ON in DMF (3 mL) at 60° C. 3-Bromopiperidine-2,6-dione (0.54 g) and NaHCO₃ (0.18 g) were added and stirring continued ON at 60° C. The OL (EtOAc/water) was washed with brine, dried, concentrated, and purified by FC (pentane/EtOAc 1:1 to 0:1) to give Int. 2M12 tert-butyl 4-((4-(4-((2,6-dioxopiperidin-3-yl)amino)-2-fluoro-phenyl)-3,3-difluoropiperidin-1-yl)methyl)-piperidine-1-carboxylate (0.14 g). This material was stirred 2 h in DCM/4M HCl in dioxane (1:1, 4 mL) at 0° C. to RT, concentrated, and triturated in pentane/Et₂O to give Int. 2M7 3-((4-(3,3-difluoro-1-(piperidin-4-ylmethyl)-piperidin-4-yl)-3-fluorophenyl)amino)-piperidine-2,6-dione (0.11 g, HCl salt).

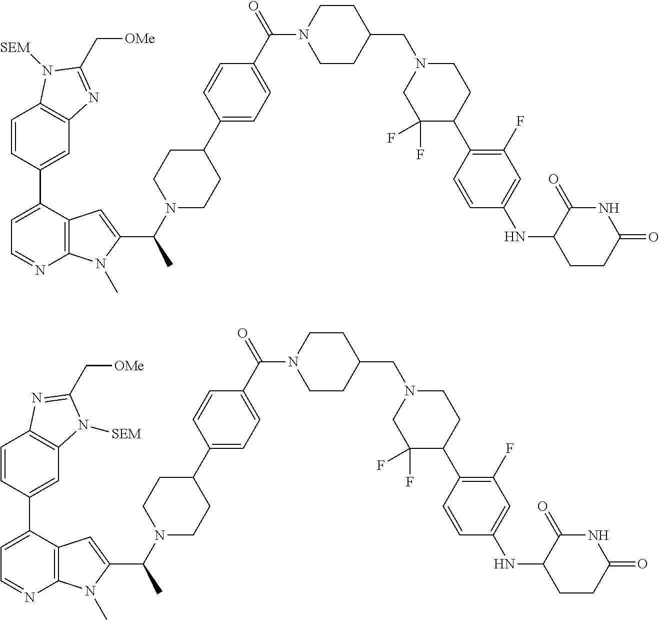 [SMILES: COCc1nc2cc(-c3ccnc4c3cc([C@H](C)N3CCC(c5ccc(C(=O)N6CCC(CN7CCC(c8ccc(NC9CCC(=O)NC9=O)cc8F)C(F)(F)C7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCC(c8ccc(NC9CCC(=O)NC9=O)cc8F)C(F)(F)C7)CC6)cc5)CC3)n4C)cc2n1COCC[Si](C)(C)C]

Int. 2M13/2M14 3-((4-(3,3-difluoro-1-((1-(4-(1-((S)-1-(4-(2-(methoxymethyl)-1-((2-(trimethylsilyl) ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperidin-4-yl)-3-fluorophenyl)amino)-piperidine-2,6-dione and 3-((4-(3,3-difluoro-1-((1-(4-(1-((S)-1-(4-(2-(methoxymethyl)-1-((2-(trimethylsilyl) ethoxy)-methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperidin-4-yl)-3-fluorophenyl)-amino)-piperidine-2,6-dione (0.20 g) prepared similarly to Int. 2C18 from Ints. 2F8/2F9/2M7 (0.20 g/0.24 g, HCl salt).

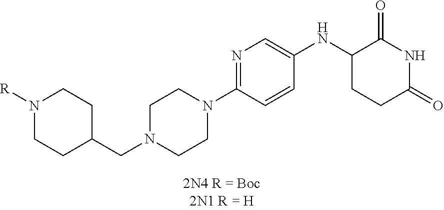

Int. 2N1 3-((6-(4-(piperidin-4-ylmethyl)piperazin-1-yl)pyridin-3-yl)amino)piperidine-2,6-dione (20 mg, TFA salt) prepared similarly to Int. 2L1 from Int. 2N2 (0.40 g).

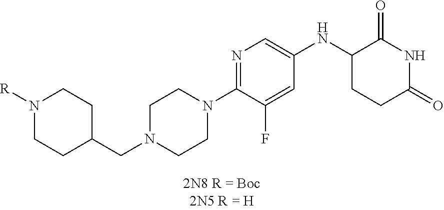

Int. 2N5 3-((5-fluoro-6-(4-(piperidin-4-ylmethyl)piperazin-1-yl)-pyridin-3-yl)amino)-piperidine-2,6-dione (7.5 g, HCl salt) was prepared from Int. 2N6 similarly to Int. 2L1 using 4M HCl in dioxane instead of TFA.

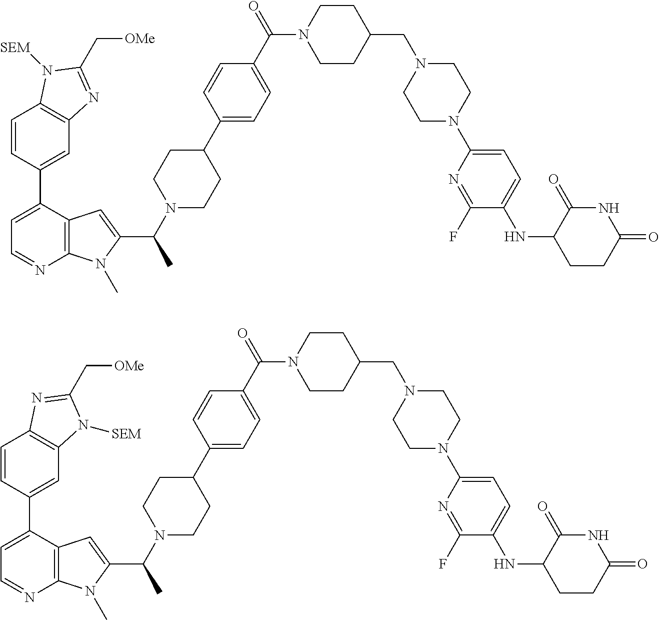 [SMILES: COCc1nc2cc(-c3ccnc4c3cc([C@H](C)N3CCC(c5ccc(C(=O)N6CCC(CN7CCN(c8ccc(NC9CCC(=O)NC9=O)c(F)n8)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ccc(NC9CCC(=O)NC9=O)c(F)n8)CC7)CC6)cc5)CC3)n4C)cc2n1COCC[Si](C)(C)C]

Int. 2N11/2N12 3-((2-fluoro-6-(4-((1-(4-(1-((S)-1-(4-(2-(methoxymethyl)-1-((2-(trimethylsilyl)-ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperazin-1-yl)pyridin-3-yl)amino)piperidine-2,6-dione and 3-((2-fluoro-6-(4-((1-(4-(1-((S)-1-(4-(2-(methoxymethyl)-1-((2-(trimethylsilyl) ethoxy)methyl)-1H-benzo[d] imidazol-6-yl)-1-methyl-1H-pyrrolo[2,3-b]pyridin-2-yl)ethyl)piperidin-4-yl)benzoyl)piperidin-4-yl)methyl)piperazin-1-yl)pyridin-3-yl)-amino)piperidine-2,6-dione (80 mg) prepared similarly to Int. 2C18 from Ints. 2F8/2F9/2N14 (0.10 g/88 mg, HCl salt).

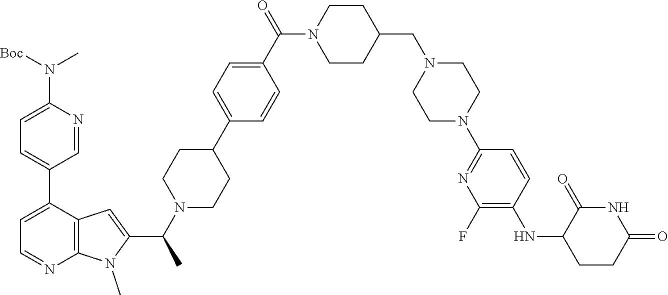 [SMILES: C[C@@H](c1cc2c(-c3ccc(N(C)C(=O)OC(C)(C)C)nc3)ccnc2n1C)N1CCC(c2ccc(C(=O)N3CCC(CN4CCN(c5ccc(NC6CCC(=O)NC6=O)c(F)n5)CC4)CC3)cc2)CC1]

nt. 2N13 tert-butyl (5-(2-((1S)-1-(4-(4-(4-((4-(5-((2,6-dioxopiperidin-3-yl)amino)-6-fluoropyridin-2-yl) piperazin-1-yl)methyl)piperidine-1-carbonyl)phenyl) piperidin-1-yl)ethyl)-1-methyl-1H-pyrrolo[2,3-b]pyridin-4-yl)pyridin-2-yl) (methyl) carbamate (0.20 g) prepared similarly to Int. 2C18 from Int. 2N9/2N14 (0.20 g/0.22 g, HCl salt).

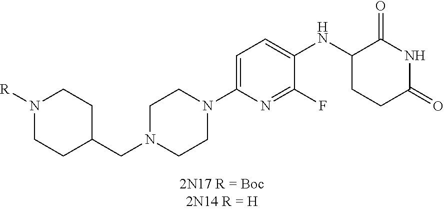

Int. 2N14 3-((2-fluoro-6-(4-(piperidin-4-ylmethyl)-piperazin-1-yl)pyridin-3-yl)amino)piperidine-2,6-dione (2.5 g, HCl salt) was prepared from Int. 2N15 similarly to Int. 2N5.

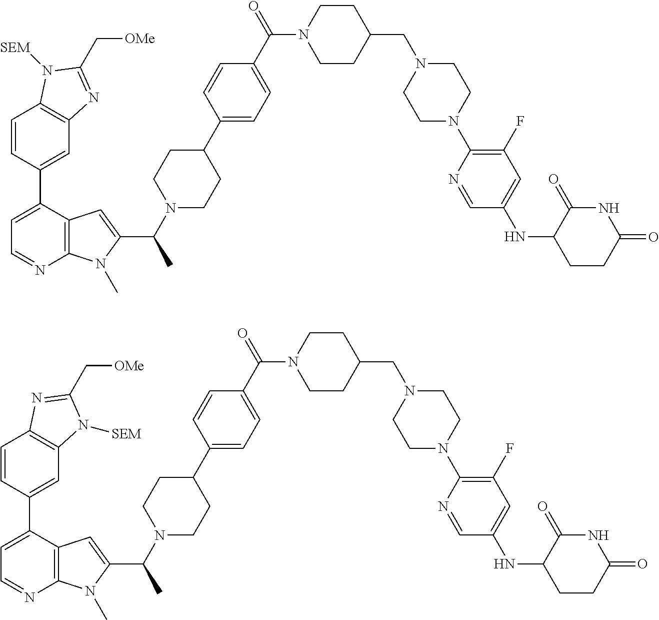 [SMILES: COCc1nc2cc(-c3ccnc4c3cc([C@H](C)N3CCC(c5ccc(C(=O)N6CCC(CN7CCN(c8ncc(NC9CCC(=O)NC9=O)cc8F)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ncc(NC9CCC(=O)NC9=O)cc8F)CC7)CC6)cc5)CC3)n4C)cc2n1COCC[Si](C)(C)C]

Int. 2N18/2N19 3-((5-fluoro-6-(4-((1-(4-(1-((S)-1-(4-(2-(methoxymethyl)-1-((2-(trimethylsilyl)-ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperazin-1-yl)pyridin-3-yl)amino)piperidine-2,6-dione and 3-((5-fluoro-6-(4-((1-(4-(1-((S)-1-(4-(2-(methoxymethyl)-1-((2-(trimethylsilyl) ethoxy)methyl)-1H-benzo[d] imidazol-6-yl)-1-methyl-1H-pyrrolo[2,3-b]pyridin-2-yl)ethyl)piperidin-4-yl)benzoyl)piperidin-4-yl)methyl)piperazin-1-yl)pyridin-3-yl)amino)-piperidine-2,6-dione (0.10 g) prepared similarly to Int. 2C18 from Ints. 2F8/2F9/2N5 (80 mg/76 mg, HCl salt).

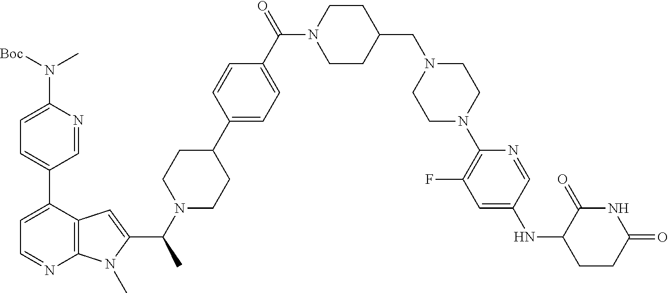 [SMILES: C[C@@H](c1cc2c(-c3ccc(N(C)C(=O)OC(C)(C)C)nc3)ccnc2n1C)N1CCC(c2ccc(C(=O)N3CCC(CN4CCN(c5ncc(NC6CCC(=O)NC6=O)cc5F)CC4)CC3)cc2)CC1]

Int. 2N20 tert-butyl (5-(2-((1S)-1-(4-(4-(4-((4-(5-((2,6-dioxopiperidin-3-yl)amino)-3-fluoropyridin-2-yl) piperazin-1-yl)methyl)piperidine-1-carbonyl)phenyl) piperidin-1-yl)ethyl)-1-methyl-1H-pyrrolo[2,3-b]pyridin-4-yl)pyridin-2-yl) (methyl) carbamate (0.20 g) prepared similarly to Int. 2C18 from Ints. 2N9/2N5 (0.15 g/0.16 g, HCl salt).

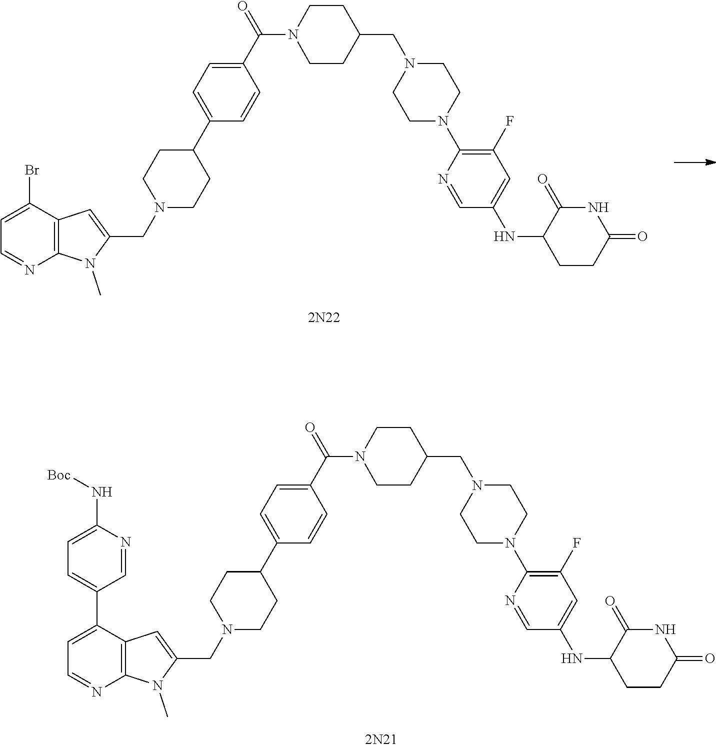 [SMILES: Cn1c(CN2CCC(c3ccc(C(=O)N4CCC(CN5CCN(c6ncc(NC7CCC(=O)NC7=O)cc6F)CC5)CC4)cc3)CC2)cc2c(Br)ccnc21 ; Cn1c(CN2CCC(c3ccc(C(=O)N4CCC(CN5CCN(c6ncc(NC7CCC(=O)NC7=O)cc6F)CC5)CC4)cc3)CC2)cc2c(-c3ccc(NC(=O)OC(C)(C)C)nc3)ccnc21]

Int. 2N22 3-((6-(4-((1-(4-(1-((4-bromo-1-methyl-1H-pyrrolo[2,3-b]pyridin-2-yl)methyl)-piperidin-4-yl)benzoyl)-piperidin-4-yl)methyl)piperazin-1-yl)-5-fluoropyridin-3-yl)amino)piperidine-2,6-dione (57 mg) was prepared similarly to Int. 2C18 from Ints. 1AC4/2N5 (70 mg/86 mg, HCl salt). tert-Butyl (5-(4,4,5,5-tetramethyl-1,3,2-dioxaborolan-2-yl)pyridin-2-yl)-carbamate (29 mg), Int. 2N22 (53 mg), and PdDPPFCl₂-DCM (5 mg) were degassed in DMF/10% aq. Na₂CO₃ (5:1, 0.84 mL) and stirred 0.5 h at 100° C. The OL (EtOAc/water) was washed with brine, dried, concentrated, and purified by FC (DCM/MeOH 1:0 to 9:1) to give Int. 2N21 tert-butyl (5-(2-((4-(4-(4-((4-(5-((2,6-dioxopiperidin-3-yl)amino)-3-fluoropyridin-2-yl) piperazin-1-yl)-methyl)piperidine-1-carbonyl)phenyl) piperidin-1-yl)methyl)-1-methyl-1H-pyrrolo[2,3-b]pyridin-4-yl)pyridin-2-yl) carbamate (20 mg).

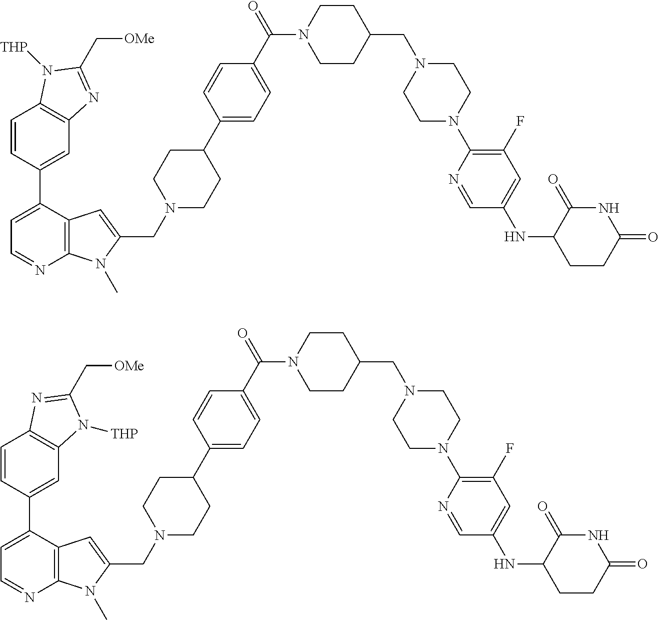

Int. 2N23/2N24 3-((5-fluoro-6-(4-((1-(4-(1-((4-(2-(methoxymethyl)-1-(tetrahydro-2H-pyran-2-yl)-1H-benzo[d]imidazol-5-yl)-1-methyl-1H-pyrrolo[2,3-b]pyridin-2-yl)methyl)--piperidin-4-yl)benzoyl)piperidin-4-yl)methyl)piperazin-1-yl)pyridin-3-yl)amino)piperidine-2,6-dione and 3-((5-fluoro-6-(4-((1-(4-(1-((4-(2-(methoxymethyl)-1-(tetrahydro-2H-pyran-2-yl)-1H-benzo[d]imidazol-6-yl)-1-methyl-1H-pyrrolo[2,3-b]pyridin-2-yl)methyl)piperidin-4-yl)benzoyl)piperidin-4-yl)methyl)piperazin-1-yl)pyridin-3-yl)amino)piperidine-2,6-dione (15 mg) prepared similarly to Int. 2C18 from Ints. 2C55/2N5 (29 mg/21 mg, HCl salt).

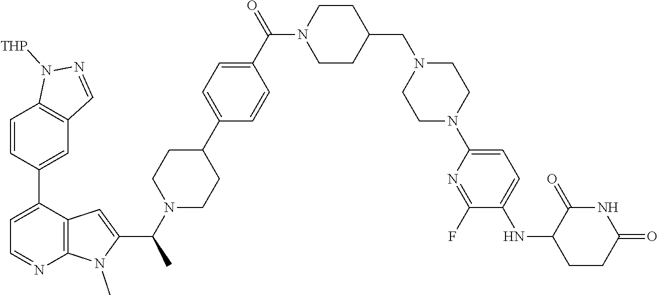

Int. 2N25 3-((2-fluoro-6-(4-((1-(4-(1-((1S)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoyl)piperidin-4-yl)methyl)piperazin-1-yl)pyridin-3-yl)amino)piperidine-2,6-dione and 3-((2-fluoro-6-(4-((1-(4-(1-((1S)-1-(4-(2-(methoxymethyl)-1-(tetrahydro-2H-pyran-2-yl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)pyridin-3-yl)amino)piperidine-2,6-dione (106 mg) prepared similarly to Int. 2C18 from Int. 2C54 (92 mg) and 2N14 (90 mg, HCl salt).

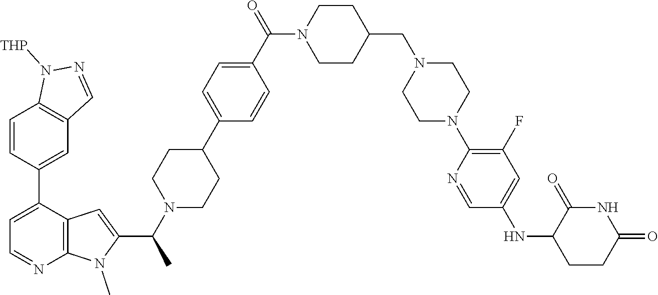

Int. 2N26 3-((5-fluoro-6-(4-((1-(4-(1-((1S)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoyl)piperidin-4-yl)methyl)piperazin-1-yl)pyridin-3-yl)amino)piperidine-2,6-dione (63 mg) prepared similarly to Int. 2C18 from Ints. 2C54/2N5 (53 mg/52 mg, HCl salt).

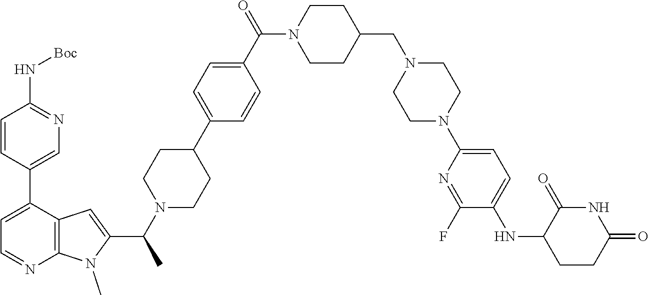 [SMILES: C[C@@H](c1cc2c(-c3ccc(NC(=O)OC(C)(C)C)nc3)ccnc2n1C)N1CCC(c2ccc(C(=O)N3CCC(CN4CCN(c5ccc(NC6CCC(=O)NC6=O)c(F)n5)CC4)CC3)cc2)CC1]

Int. 2N27 tert-butyl (5-(2-((1S)-1-(4-(4-(4-((4-(5-((2,6-dioxopiperidin-3-yl)amino)-6-fluoropyridin-2-yl) piperazin-1-yl)methyl)piperidine-1-carbonyl)phenyl) piperidin-1-yl)ethyl)-1-methyl-1H-pyrrolo[2,3-b]pyridin-4-yl)pyridin-2-yl) carbamate (104 mg) prepared similarly to Int. 2C18 from Ints. 3E76/2N14 (90 mg/89 mg, HCl salt).

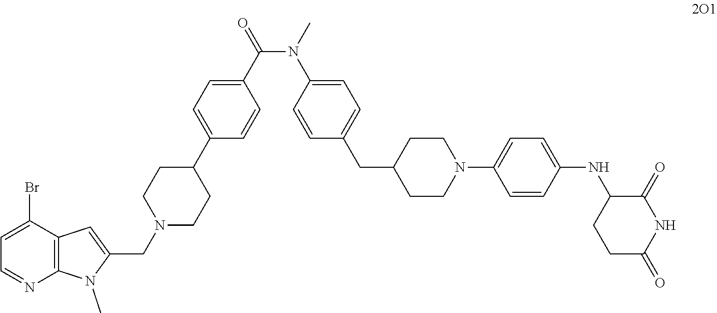 [SMILES: CN(C(=O)c1ccc(C2CCN(Cc3cc4c(Br)ccnc4n3C)CC2)cc1)c1ccc(CC2CCN(c3ccc(NC4CCC(=O)NC4=O)cc3)CC2)cc1]

Int. 2O5 (98 mg), Na₂S₂O₄ (0.12 g) were stirred 3 h in MeOH (3 mL) at 70° C. The OL (EtOAc/water) was washed with brine, dried, and concentrated to give Int. 2O5 N-(4-((1-(4-aminophenyl) piperidin-4-yl)-methyl)phenyl)-4-(1-((4-bromo-1-methyl-1H-pyrrolo[2,3-b]pyridin-2-yl)methyl)-piperidin-4-yl)-N-methyl-benzamide (88 mg). This material, 3-bromo-piperidine-2,6-dione (36 mg), and NaHCO₃ (42 mg) were stirred ON in DMF (2 mL) at 70° C. 3-Bromo-piperidine-2,6-dione (10 mg) was added stirring continued 2 h at 70° C. The OL (2MeTHF/water) was washed with brine, dried, concentrated, and purified by FC (DCM/MeOH 1:0 to 9:1) to give Int. 2O1 4-(1-((4-bromo-1-methyl-1H-pyrrolo[2,3-b]pyridin-2-yl)methyl)-piperidin-4-yl)-N-(4-((1-(4-((2,6-dioxopiperidin-3-yl)amino)phenyl) piperidin-4-yl)methyl) phenyl)-N-methylbenzamide (98 mg).

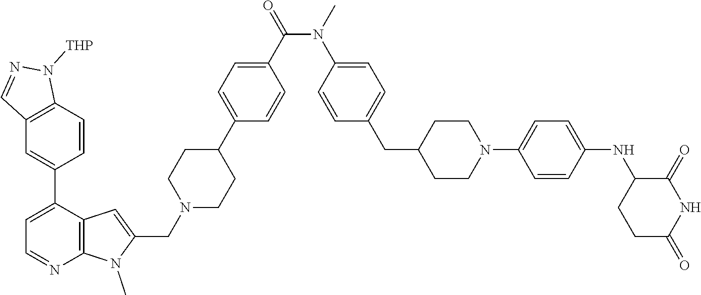

Int. 2O1 (50 mg), 1-(tetrahydro-2H-pyran-2-yl)-5-(4,4,5,5-tetramethyl-1,3,2-dioxaborolan-2-yl)-1H-indazole (19 mg), and PdDPPFCl₂-DCM (4 mg) were degassed in DMF/10% aq. Na₂CO₃ (15:1, 1.1 mL) and stirred 1.5 h at 100° C. The filtrate after filtration was purified by FC (DCM/MeOH 1:0 to 9:1) to give Int. 2O6 N-(4-((1-(4-((2,6-dioxopiperidin-3-yl)amino)phenyl) piperidin-4-yl)methyl) phenyl)-N-methyl-4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzamide (46 mg).

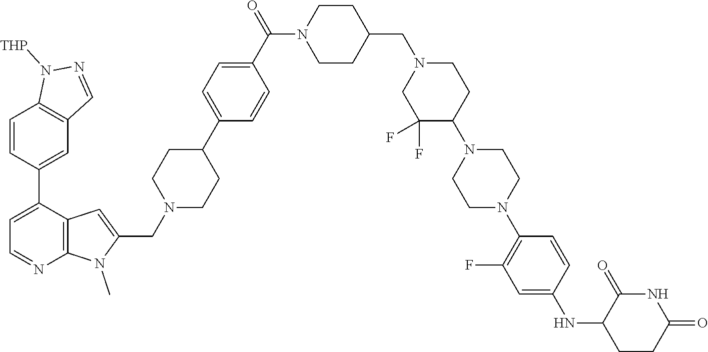

Int. 2P1 3-((4-(4-(3,3-difluoro-1-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)piperidin-4-yl) piperazin-1-yl)-3-fluorophenyl)amino)piperidine-2,6-dione (25 mg) prepared similarly to Int. 2C18 from HCl salts of Ints. 3A2/2P2 (25 mg) and 2P2 (22 mg).

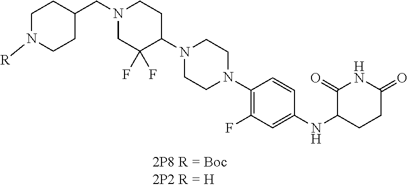

Int. 2P2 3-((4-(4-(3,3-difluoro-1-(piperidin-4-ylmethyl)piperidin-4-yl) piperazin-1-yl)-3-fluorophenyl)-amino)piperidine-2,6-dione (0.32 g, HCl salt) was prepared similarly to Int. 2H2 from Int. 2P3.

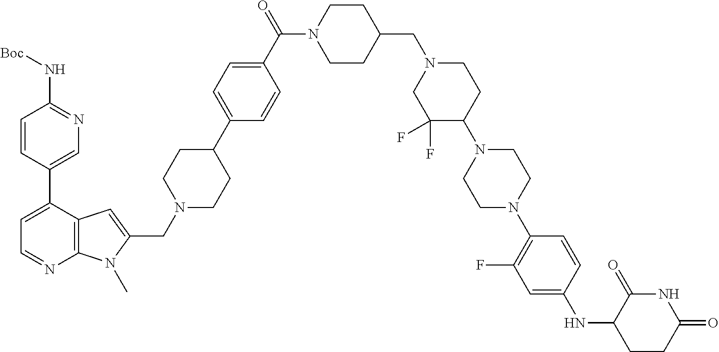 [SMILES: Cn1c(CN2CCC(c3ccc(C(=O)N4CCC(CN5CCC(N6CCN(c7ccc(NC8CCC(=O)NC8=O)cc7F)CC6)C(F)(F)C5)CC4)cc3)CC2)cc2c(-c3ccc(NC(=O)OC(C)(C)C)nc3)ccnc21]

Int. 2P9 tert-butyl (5-(2-((4-(4-(4-((4-(4-(4-((2,6-dioxopiperidin-3-yl)amino)-2-fluorophenyl) piperazin-1-yl)-3,3-difluoropiperidin-1-yl)methyl)piperidine-1-carbonyl)phenyl) piperidin-1-yl)methyl)-1-methyl-1H-pyrrolo[2,3-b]pyridin-4-yl)pyridin-2-yl) carbamate (27 mg) prepared similarly to Int. 2C18 from Int. 2E6 (20 mg, HCl salt) and 2P2 (22 mg, HCl salt).

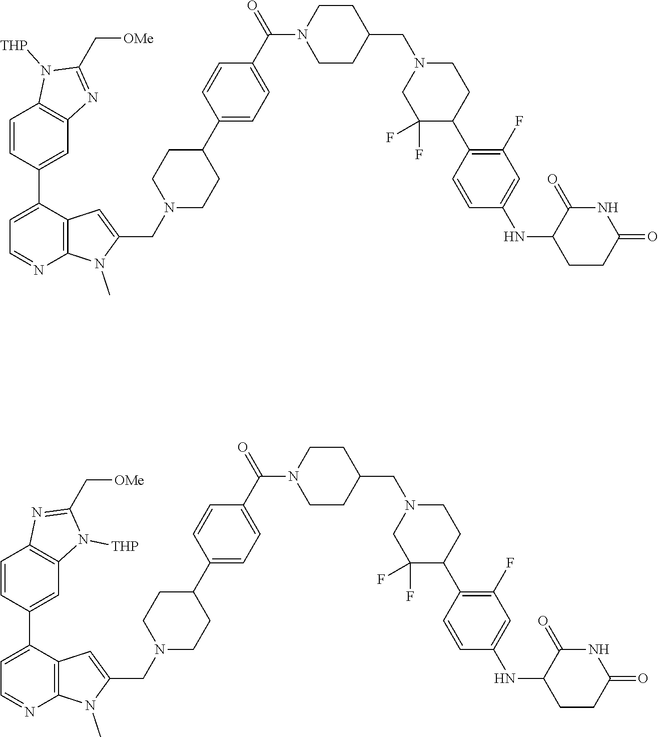

Int. 2P10/2P11 3-((4-(4-(3,3-difluoro-1-((1-(4-(1-((4-(2-(methoxymethyl)-1-(tetrahydro-2H-pyran-2-yl)-1H-benzo[d]imidazol-5-yl)-1-methyl-1H-pyrrolo[2,3-b]pyridin-2-yl)methyl)piperidin-4-yl)benzoyl)-piperidin-4-yl)-methyl)piperidin-4-yl) piperazin-1-yl)-3-fluorophenyl)amino)piperidine-2,6-dione and 3-((4-(3,3-difluoro-1-((1-(4-(1-((4-(2-(methoxymethyl)-1-(tetrahydro-2H-pyran-2-yl)-1H-benzo[d] imidazol-6-yl)-1-methyl-1H-pyrrolo[2,3-b]pyridin-2-yl)methyl)piperidin-4-yl)benzoyl)piperidin-4-yl)methyl)piperidin-4-yl)-3-fluorophenyl)amino)-piperidine-2,6-dione (20 mg) prepared similarly to Int. 2C18 from Ints. 2C55/2P2 (18 mg/19 mg, HCl salt).

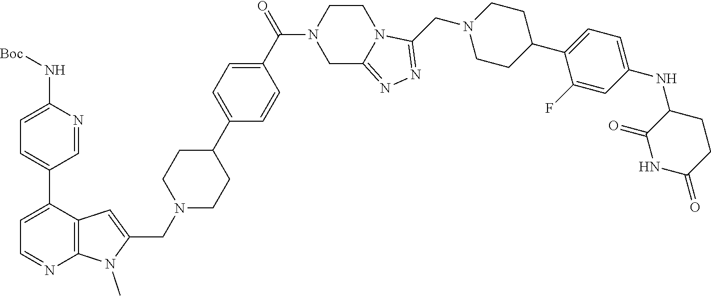 [SMILES: Cn1c(CN2CCC(c3ccc(C(=O)N4CCn5c(CN6CCC(c7ccc(NC8CCC(=O)NC8=O)cc7F)CC6)nnc5C4)cc3)CC2)cc2c(-c3ccc(NC(=O)OC(C)(C)C)nc3)ccnc21]

Int. 2Q1 tert-butyl (5-(2-((4-(4-(3-((4-(4-((2,6-dioxopiperidin-3-yl)amino)-2-fluorophenyl)-piperidin-1-yl)methyl)-5,6,7,8-tetrahydro-[1,2,4]triazolo [4,3-a]pyrazine-7-carbonyl)phenyl) piperidin-1-yl)methyl)-1-methyl-1H-pyrrolo[2,3-b]pyridin-4-yl)pyridin-2-yl) carbamate (80 mg) prepared similarly to Int. 2C18 from Ints. 2E6/2Q2 (44 mg/55 mg, HCl salt).

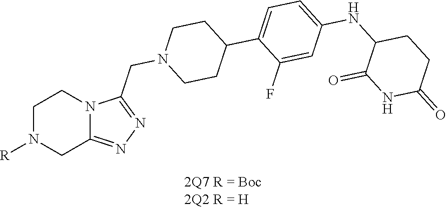

Int. 2Q6 (0.39 g), NaHCO₃ (0.23 g), and 3-bromopiperidine-2,6-dione (0.70 g) were stirred ON in DMF (4 mL) at 60° C. 3-Bromopiperidine-2,6-dione (0.39 g) was added and stirring continued ON at 60° C. The OL (EtOAc/water) was washed with brine, dried, concentrated, and purified by FC (DCM/MeOH 95:5 to 9:1) to give Int. 2Q7 tert-butyl 3-((4-(4-((2,6-dioxopiperidin-3-yl)amino)-2-fluorophenyl)-piperidin-1-yl)methyl)-5,6-dihydro-[1,2,4]triazolo [4,3-a]pyrazine-7 (8H)-carboxylate (0.13 g). This material was stirred 2 h in DCM/4M HCl in dioxane (3.8:1, 6.3 mL) at 0° C. to RT and concentrated to give Int. 2Q2 3-((3-fluoro-4-(1-((5,6,7,8-tetrahydro-[1,2,4]-triazolo [4,3-a]pyrazin-3-yl)methyl)-piperidin-4-yl)phenyl)amino)piperidine-2,6-dione (0.11 g, HCl salt).

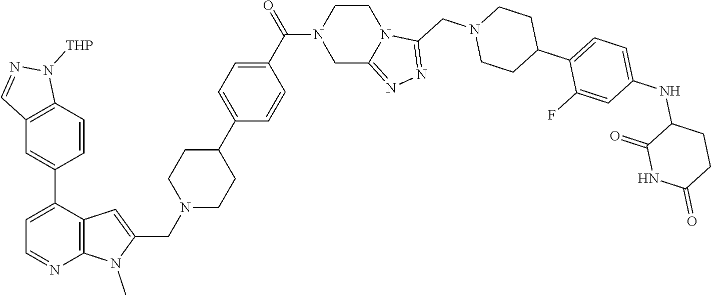

Int. 2Q9 3-((3-fluoro-4-(1-((7-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)-5,6,7,8-tetrahydro-[1,2,4]triazolo [4,3-a]pyrazin-3-yl)methyl)-piperidin-4-yl)phenyl)amino)piperidine-2,6-dione (0.12 g) prepared similarly to Int. 2C18 from Ints. 3A2 (44 mg) and 2Q2 (55 mg, HCl salt).

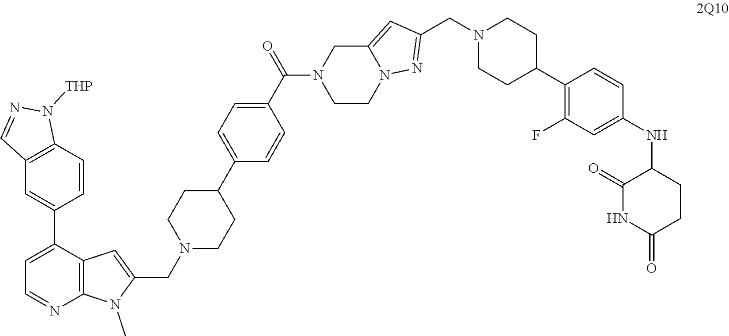

Int. 2Q10 3-((3-fluoro-4-(1-((5-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)-4,5,6,7-tetra-hydropyrazolo [1,5-a]pyrazin-2-yl)methyl)piperidin-4-yl)phenyl)amino)piperidine-2,6-dione (60 mg) prepared similarly to Int. 2C18 from Ints. 3A2/2Q15(46 mg/40 mg, HCl salt).

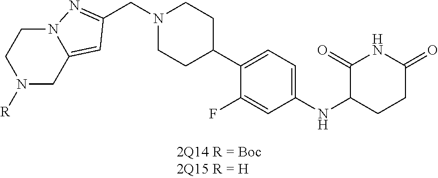

Int. 2Q15 3-((3-fluoro-4-(1-((4,5,6,7-tetra-hydropyrazolo-[1,5-a]pyrazin-2-yl)methyl)-piperidin-4-yl)phenyl)-amino)piperidine-2,6-dione (80 mg, HCl salt) was prepared from Int. 2Q12 similarly to Int. 2Q2.

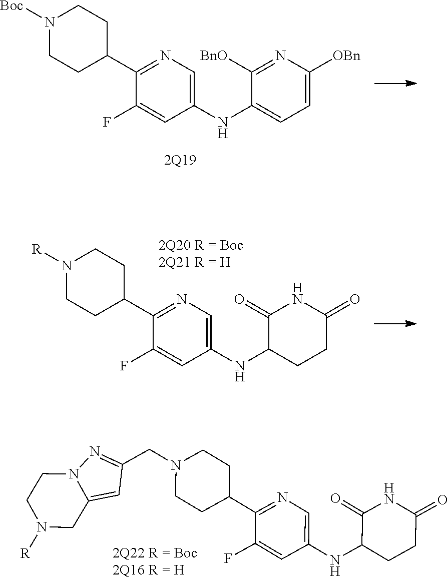

Int. 2Q18 (1.0 g), 2,6-bis(benzyloxy)-3-bromo-pyridine (1.6 g), Cs₂CO₃ (1.8 g), and Brettphos PdG₃ (0.18 g) were degassed in toluene (8 mL) and stirred ON at 100° C. and filtered. The OL (EtOAc/water) was dried, concentrated, and purified by FC (pentane/EtOAc 1:0 to 3:1) to give Int. 2Q19 tert-butyl 4-(5-((2,6-bis-(benzyloxy)-pyridin-3-yl)amino)-3-fluoropyridin-2-yl)-piperidine-1-carboxylate (1.4 g). Int. 2Q19 (1.3 g) was hydrogenated ON using 10% Pd/C (1.0 g) in EtOAc (20 mL), filtered, concentrated, and purified by FC (pentane/EtOAc 1:0 to 2:3) to give Int. 2Q20 tert-butyl 4-(5-((2,6-dioxopiperidin-3-yl)amino)-3-fluoro-pyridin-2-yl) piperidine-1-carboxylate (0.65 g). 0.55 g of this was stirred 8 h in DCM/TFA (2:1, 15 mL) and concentrated to give Int. 2Q21 3-((5-fluoro-6-(piperidin-4-yl)pyridin-3-yl)amino)-piperidine-2,6-dione (0.5 g, TFA salt). Int. 2Q21 (0.45 g) and Int. 2Q11 (0.3 g) were stirred 6 h in DCE/THF/DIPEA (10:10:1, 21 mL). STAB (0.45 g) was added at 0° C. before stirring ON at 0° C. to RT. Th OL (EtOAc/water) was dried, concentrated, and purified by FC (DCM/MeOH 1:0 to 9:1) to give Int. 2Q22 tert-butyl 2-((4-(5-((2,6-dioxopiperidin-3-yl)-amino)-3-fluoropyridin-2-yl) piperidin-1-yl)methyl)-6,7-dihydropyrazolo[1,5-a]-pyrazine-5(4H)-carboxylate (0.28 g). This material was stirred 6 h in 2M HCl dioxane (12 mL) and concentrated to give Int. 2Q16 3-((5-fluoro-6-(1-((4,5,6,7-tetrahydropyrazolo [1,5-a]pyrazin-2-yl)methyl)piperidin-4-yl)pyridin-3-yl)amino)-piperidine-2,6-dione (0.20 g, HCl salt).

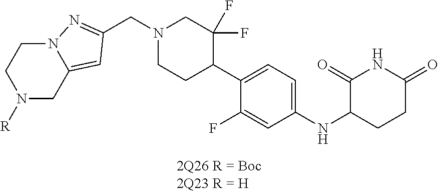

Int. 2Q23 3-((4-(3,3-difluoro-1-((4,5,6,7-tetrahydropyrazolo [1,5-a]pyrazin-2-yl)methyl)piperidin-4-yl)-3-fluorophenyl)amino)piperidine-2,6-dione (80 mg, HCl salt) was prepared from Int. 2Q24 similarly to Int. 2M7.

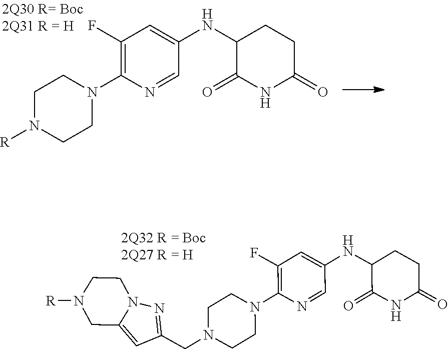

Int. 2Q29 (1.4 g), 3-bromopiperidine-2,6-dione (2.7 g), and NaHCO₃ (0.6 g) were stirred ON in DMF (5 mL) at 50° C. 3-Bromo-piperidine-2,6-dione (2.7 g) and NaHCO₃ (0.4 g) were added and stirring continued ON at 50° C., filtered, concentrated, and purified by FC (pentane/EtOAc 1:1 to 2:3) to give Int. 2Q30 tert-butyl 4-(5-((2,6-dioxopiperidin-3-yl)amino)-3-fluoropyridin-2-yl) piperazine-1-carboxylate (1.2 g). Int. 2Q30 (1.1 g) was stirred 3 h in 2.8M HCl in dioxane (16 mL) at 0° C. to RT, concentrated, and triturated in Et₂O/pentane to give Int. 2Q31 3-((5-fluoro-6-(piperazin-1-yl)pyridin-3-yl)amino)piperidine-2,6-dione (0.8 g, HCl salt). Ints. 2Q31/Int. 2Q11 (0.8 g/0.5 g) were stirred ON in DCE/DIPEA (1:2, 3 mL). STAB (1.3 g) was added and stirring continued ON at 0° to RT. The OL (water/DCM) was concentrated and purified by FC (DCM/MeOH 9:1 to 4:1) to give Int. 2Q32 tert-butyl 2-((4-(5-((2,6-dioxopiperidin-3-yl)amino)-3-fluoro-pyridin-2-yl) piperazin-1-yl)-methyl)-6,7-dihydropyrazolo [1,5-a]pyrazine-5(4H)-carboxylate (0.7 g). This material was stirred 3 h in 2.8M HCl in dioxane (10 mL) at 0° C. to RT, concentrated, and triturated in Et₂O/pentane to give Int. 2Q27 3-((5-fluoro-6-(4-((4,5,6,7-tetrahydro-pyrazolo [1,5-a]pyrazin-2-yl)methyl)piperazin-1-yl)pyridin-3-yl)amino)-piperidine-2,6-dione (0.4 g, HCl salt).

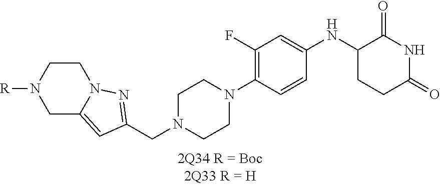

Int. 2Q33. 3-((3-fluoro-4-(4-((4,5,6,7-tetrahydropyrazolo [1,5-a]pyrazin-2-yl)methyl)-piperazin-1-yl)phenyl)-amino)piperidine-2,6-dione (0.5 g, HCl salt) was prepared similarly to Int. 2Q27 from Int. 2A7.

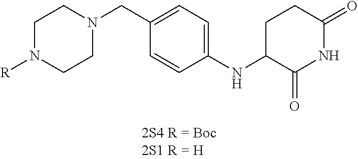

Int. 2S3 (70 mg), 3-bromopiperidine-2,6-dione (69 mg), and NaHCO₃ (81 mg) in DMF (1 mL) were stirred ON at 70° C. The OL (2MeTHF/water) was washed with brine, dried, concentrated, and purified by FC (DCM/MeOH 1:0 to 9:1) to give Int. 2S4 tert-butyl 4-(4-((2,6-dioxo-piperidin-3-yl)amino) benzyl) piperazine-1-carboxylate (96 mg), which was stirred 0.5 h in DCM/TFA (4:1, 2.5 mL) and concentrated to give Int. 2S1 3-((4-(piperazin-1-ylmethyl)-phenyl)amino)piperidine-2,6-dione (96 mg, TFA salt).

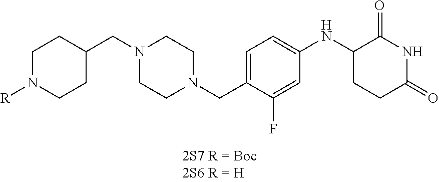

Int. 2S8 (75 mg), NaHCO₃ (62 mg), and 3-bromopiperidine-2,6-dione (53 mg) were stirred ON in DMF (2 mL) at 60° C. 3-Bromopiperidine-2,6-dione (50 mg) was added and stirring continued 4 days at 80° C. The OL (water/EtOAc) was washed with brine, dried, concentrated, and purified by FC (DCM/MeOH/Et₃N 95:4.5:0.5) to give Int. 2S7 tert-butyl 4-[[4-[[2-fluoro-4-[1-[(4-methoxyphenyl)methyl]-2,6-dioxo-3-piperidyl]amino]phenyl]methyl]piperazin-1-yl]-methyl]piperidine-1-carboxylate (34 mg). This material was stirred 0.5 h in DCM/TFA (4:1, 2.5 mL) and concentrated to give Int. 2S6 3-((3-fluoro-4-((4-(piperidin-4-ylmethyl)piperazin-1-yl)methyl) phenyl)amino)piperidine-2,6-dione (33 mg, TFA salt).

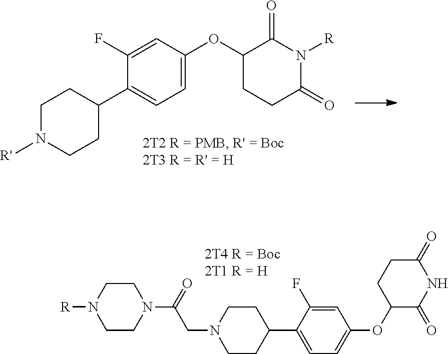

3-Bromo-1-(4-methoxybenzyl) piperidine-2,6-dione (198 mg), Cs₂CO₃ (0.38 g), and tert-butyl 4-(2-fluoro-4-hydroxyphenyl) piperidine-1-carboxylate (0.17 g) were stirred ON in DMF (3 mL). The OL (EtOAc/water) was washed with brine, concentrated, and HPLC-purified to give Int. 2T2 tert-butyl 4-(2-fluoro-4-((1-(4-methoxy-benzyl)-2,6-dioxo-piperidin-3-yl)oxy) phenyl)-piperidine-1-carboxylate (53 mg). This material was stirred 0.5 h in TFA/TfOH (5:1, 12 mL) at 80° C. and concentrated. The OL (EtOAc/sat. aq. NaHCO₃) was washed with water and brine, dried, concentrated, and HPLC-purified to give Int. 2T3 3-(3-fluoro-4-(piperidin-4-yl)-phenoxy) piperidine-2,6-dione (15 mg). This material and tert-butyl 4-(2-bromoacetyl)-piperazine-1-carboxylate (17 mg) were stirred 2 h in ACN/DIPEA (10:1, 1.1 mL) at 66° C. The residue after concentration was stirred 1 h in DCM/TFA (2:1, 6 mL) and concentrated to give Int. 2T1 3-(3-fluoro-4-(1-(2-oxo-2-(piperazin-1-yl)ethyl)piperidin-4-yl) phenoxy) piperidine-2,6-dione (15 mg, TFA salt).

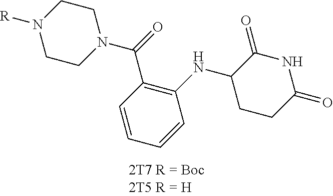

Int. 2T6 (0.31 g), 3-bromopiperidine-2,6-dione (0.29 g), and NaHCO₃ (0.34 g) were stirred 0.5 h in DMF (2 mL) at 70° C. 3-Bromopiperidine-2,6-dione (97 mg) was added and stirring was continued ON at 70° C. and concentrated. The OL (EtOAc/water) was washed with brine, dried, concentrated, and purified by FC to give Int. 2T7 tert-butyl 4-(2-((2,6-dioxo-piperidin-3-yl)amino) benzoyl) piperazine-1-carboxylate (52 mg). This material was stirred 0.5 h in DCM/TFA (1:1, 2 mL) and concentrated to give Int. 2T5 3-((2-(piperazine-1-carbonyl)phenyl)amino)piperidine-2,6-dione (56 mg, TFA salt).

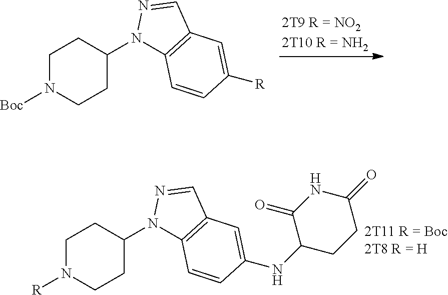

Int. 2T8 3-((1-(piperidin-4-yl)-1H-indazol-5-yl)amino)piperidine-2,6-dione (0.4 g, TFA salt) was prepared similarly to Int. 2G2 from 5-nitro-1H-indazole.

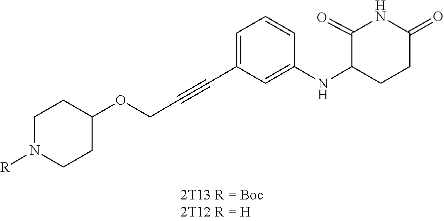

Cs₂CO₃ (133 mg), CuI (4 mg), PdCl₂ (PPh₃) 2 (14 mg), 3-((3-iodophenyl)amino)piperidine-2,6-dione (45 mg), tert-butyl 4-(prop-2-yn-1-yloxy) piperidine-1-carboxylate (49 mg), and 4 Å MS were degassed in DMF and stirred 1.5 h at 80° C. The OL (EtOAc/water) was washed brine, dried, concentrated, and HPLC-purified to give Int. 2T13 tert-butyl 4-((3-(3-((2,6-dioxopiperidin-3-yl)amino)phenyl)-prop-2-yn-1-yl)oxy) piperidine-1-carboxylate (26 mg). This material was stirred 0.5 h in DCM/TFA (1:1, 2 mL) and concentrated to give Int. 2T12 3-((3-(3-(piperidin-4-yloxy) prop-1-yn-1-yl)phenyl)amino)piperidine-2,6-dione (25 mg, TFA salt).

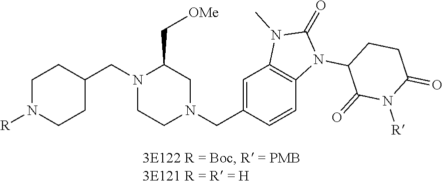

Ints. 3E3/3E123 (31 mg/50 mg) were stirred 6 h in DCE/DIPEA (12.5:1, 1.1 mL). STAB (64 mg) was added and stirring continued ON. The OL (water/DCM) was dried and concentrated. The residue was combined with another batch prepared similarly and purified by FC (hexane/EtOAc 1:4) to give Int. 3E122 tert-butyl 4-[[(2R)-2-(methoxy-methyl)-4-[1-[1-[(4-methoxy-phenyl)methyl]-2,6-dioxo-3-piperidyl]-3-methyl-2-oxo-benz-imidazol-5-yl]methyl]piperazin-1-yl]methyl]-piperidine-1-carboxylate (30 mg). This material (20 mg) was stirred 1 h in TFA/TfOH (10:1; 0.22 mL) at 80° C. and concentrated to give Int. 3E121 3-(5-(((R)-3-(methoxymethyl)-4-(piperidin-4-ylmethyl)piperazin-1-yl)methyl)-3-methyl-2-oxo-2,3-dihydro-1H-benzo[d]-imidazol-1-yl) piperidine-2,6-dione (15 mg, TfOH salt).

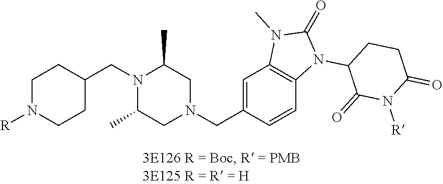

Ints. 3E3/3E127 (0.13 g/0.1 g) were stirred ON in DCE/DIPEA (14.7:1, 5.3 mL). STAB (0.27 g) was added and stirring continued ON. The OL (water/DCM/MeOH (9:1)) was washed with brine, dried, and concentrated to give Int. 3E126 tert-butyl 4-[(2S,6S)-4-[1-[1-[(4-methoxyphenyl)methyl]-2,6-dioxo-3-piperidyl]-3-methyl-2-oxo-benzimidazol-5-yl]-methyl]-2,6-dimethyl-piperazin-1-yl]methyl]-piperidine-1-carboxylate (0.15 g). This material was stirred 4 h in TFA/TfOH (5:2; 1.2 mL) at 80° C., concentrated, and triturated in Et₂O to give Int. 3E125 3-(5-(((3S,5S)-3,5-dimethyl-4-(piperidin-4-ylmethyl)-piperazin-1-yl)methyl)-3-methyl-2-oxo-2,3-dihydro-1H-benzo[d]imidazol-1-yl)-piperidine-2,6-dione (0.1 g, TfOH salt).

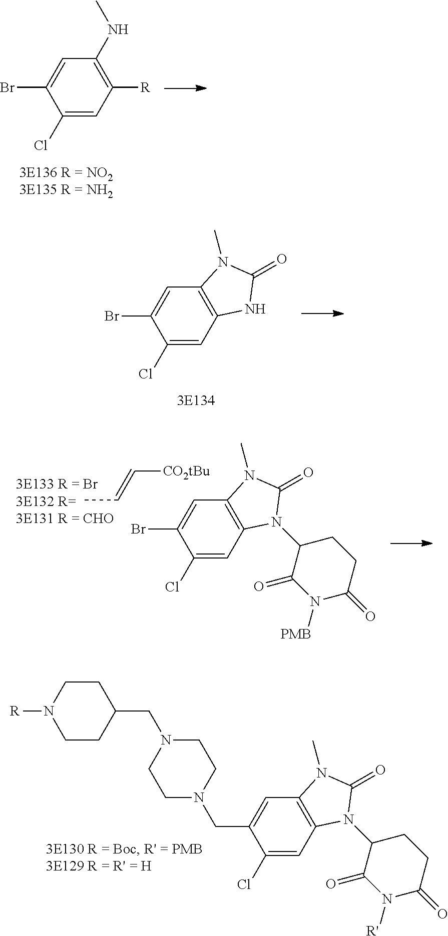

1-Bromo-2-chloro-5-fluoro-4-nitro-benzene (5.0 g) was stirred 4 h in 2M H2NMe in THF (19.6 mL) at 0° C. to RT, concentrated, and triturated in pentane to give Int. 3E136 5-bromo-4-chloro-N-methyl-2-nitro-aniline (4.5 g). This material, NH₄Cl (9.1 g), and Iron powder (5.5 g) were stirred 3 h in EtOH/water (3.5:1, 45 mL) at 70-80° C. and filtered. The OL (water/EtOAc) was dried and concentrated to afford Int. 3E135 4-bromo-5-chloro-N2-methyl-benzene-1,2-diamine (4.0 g). 1.5 g of this material and CDI (2.1 g) were stirred ON in ACN (15 mL) at 80° C., and diluted with water to precipitate Int. 3E134 5-bromo-6-chloro-3-methyl-1H-benzimidazol-2-one (1.4 g). This material and KOtBu (1.2 g) were stirred 1 h in THF (30 mL) at 0° C. Int. 3E3 (3.1 g) was added and stirring continued 6 h at 0° C. The OL (water/EtOAc) was dried, concentrated, and purified by FC (pentane/EtOAc 1:1 to 2:3) to give Int. 3E133 3-(5-bromo-6-chloro-3-methyl-2-oxo-benz-imidazol-1-yl)-1-[(4-methoxyphenyl)methyl]-piperidine-2,6-dione (1.3 g). Int. 3E133 (2.0 g), tert-butyl acrylate (1.6 g), Pd (OAc) 2 (0.22 g), and P (o-tol)₃ (0.25 g) were degassed in DMF/Et₃N (11.8:1, 22 mL) and stirred ON at 110° C. The OL (water/EtOAc) was washed with brine, dried, concentrated, and purified by FC (pentane/EtOAc 7:3) to give Int. 3E132 tert-butyl (E)-3-(6-chloro-1-(1-(4-methoxy-benzyl)-2,6-dioxo-piperidin-3-yl)-3-methyl-2-oxo-2,3-dihydro-1H-benzo[d]imidazol-5-yl) acrylate (1.6 g). This material, NaIO₄ (1.9 g), and OsO₄ (0.15 g) were stirred ON in THF/water (1:1; 20 mL). The OL (sat. aq. Na₂S₂O₃/sat. aq. NaHCO₃/EtOAc) was washed with brine, dried, concentrated, and purified by FC (pentane/EtOAc 3:7) to give Int. 3E131 6-chloro-1-(1-(4-methoxybenzyl)-2,6-dioxopiperidin-3-yl)-3-methyl-2-oxo-2,3-dihydro-1H-benzo[d] imidazole-5-carbaldehyde (1.1 g). Ints. 3E5/3E131 (0.1 g/0.1 g) were stirred ON in DCE/DIPEA (16.7:1, 2.1 mL). STAB (0.1 g) was added and stirring continued ON at 0° C. to RT. The OL (water/DCM) was dried, concentrated, and purified by FC (DCM/MeOH 95:5) to give Int. 3E130 tert-butyl 4-[4-[6-chloro-1-[1-[(4-methoxyphenyl)-methyl]-2,6-dioxo-3-piperidyl]-3-methyl-2-oxo-benzimidazol-5-yl]-methyl]piperazin-1-yl]-methyl]piperidine-1-carboxylate (trideuterio-methyl)-benzimidazol-5-yl]methyl]piperazin-1-yl]-methyl]piperidine-1-carboxylate (0.1 g). Int. 3E130 (0.26 g) was stirred in TFA/TfOH (1:0.13; 2.26 mL) at 80° C. and concentrated to give Int. 3E129 3-(6-chloro-3-methyl-2-oxo-5-((4-(piperidin-4-ylmethyl)piperazin-1-yl)-methyl)-2,3-dihydro-1H-benzo[d]imidazol-1-yl) piperidine-2,6-dione (0.3 g, TfOH salt).

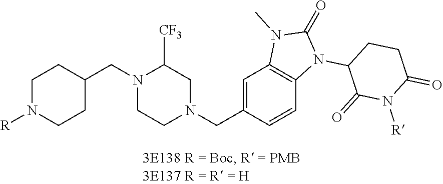

Ints. 3E3/3E139 (0.30 g/0.28 g) were stirred ON in MeOH (5 mL) and 1M ZnCl₂ in THF (1.5 mL). STAB was added and stirring continued ON. The OL (DCM/MeOH (9:1)/water) was dried, concentrated, and purified by FC (DCM/MeOH 9:1 to give Int. 3E138 tert-butyl 4-[[4-[1-[1-[(4-methoxyphenyl)-methyl]-2,6-dioxo-3-piperidyl]-3-methyl-2-oxo-benz-imidazol-5-yl]methyl]-2-(trifluoromethyl)piperazin-1-yl]methyl]piperidine-1-carboxylate (0.3 g). A solution of this material (0.2 g) in TFA/TfOH (10:1; 3.3 mL) was stirred 5 h at 80° C. and concentrated to give Int. 3E137 3-(3-methyl-2-oxo-5-((4-(piperidin-4-yl-methyl)-3-(trifluoromethyl)-piperazin-1-yl)methyl)-2,3-dihydro-1H-benzo[d] imidazol-1-yl) piperidine-2,6-dione (0.1 g, TfOH salt).

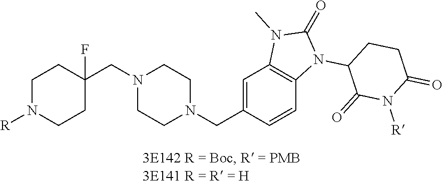

Ints. 3E3/3E143 (1.0 g/0.96 g) were stirred 6 h in DCE/DIPEA (8.8:1, 16.7 mL). STAB (1.0 g) was added and stirring continued ON at 0° C. to RT. The OL (DCM/water) was dried and concentrated. The residue was combined with another batch prepared similarly and purified by FC (pentane/EtOAc 1:4 to 1:9) to give Int. 3E142 tert-butyl 4-fluoro-4-[[4-[1-[1-[(4-methoxy-phenyl)methyl]-2,6-dioxo-3-piperidyl]-3-methyl-2-oxo-benzimidazol-5-yl]methyl]piperazin-1-yl]methyl]-piperidine-1-carboxylate (1.12 g). 0.3 g of this material was stirred 4 h in TFA/TfOH (10:1; 5.5 mL) at 60° C. and concentrated to give Int. 3E141 3-(5-((4-((4-fluoro-piperidin-4-yl)methyl)piperazin-1-yl)methyl)-3-methyl-2-oxo-2,3-dihydro-1H-benzo[d]imidazol-1-yl)-piperidine-2,6-dione (0.20 g, TfOH salt).

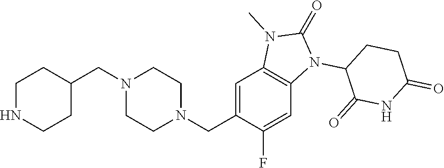 [SMILES: Cn1c(=O)n(C2CCC(=O)NC2=O)c2cc(F)c(CN3CCN(CC4CCNCC4)CC3)cc21]

Int. 3E60 (0.25 g) was stirred 3 h in TFA/TfOH (4:0.3; 4.3 mL) at 80° C. and concentrated to give Int. 3E145 3-(6-fluoro-3-methyl-2-oxo-5-((4-(piperidin-4-yl-methyl)piperazin-1-yl)methyl)-2,3-dihydro-1H-benzo[d]-imidazol-1-yl) piperidine-2,6-dione (0.20 g, TfOH salt).

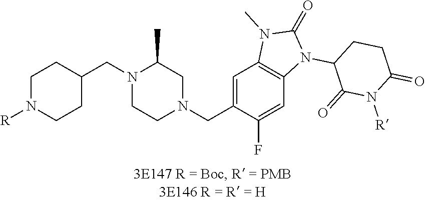

Ints. 3E61/3E74 (0.70 g/0.73 g) were stirred in DCE/DMSO/DIPEA (5:1:0.75; 13.5 mL). STAB (0.7 g) was added and stirring continued 48 h at 0° C. to RT. The OL (water/DCM) was dried, concentrated, and purified by FC (DCM/MeOH 1:0 to 95:5) to give Int. 3E147 tert-butyl 4-(((2S)-4-((6-fluoro-1-(1-(4-methoxy-benzyl)-2,6-dioxo-piperidin-3-yl)-3-methyl-2-oxo-2,3-dihydro-1H-benzo[d] imidazol-5-yl)methyl)-2-methyl-piperazin-1-yl)methyl)piperidine-1-carboxylate (0.75 g). This material was stirred 3 h in TFA/TfOH (10:1; 11 mL) at 80° C. and concentrated to give Int. 3E146 3-(6-fluoro-3-methyl-5-(((S)-3-methyl-4-(piperidin-4-ylmethyl)-piperazin-1-yl)methyl)-2-oxo-2,3-dihydro-1H-benzo[d]imidazol-1-yl) piperidine-2,6-dione (0.7 g, TfOH salt).

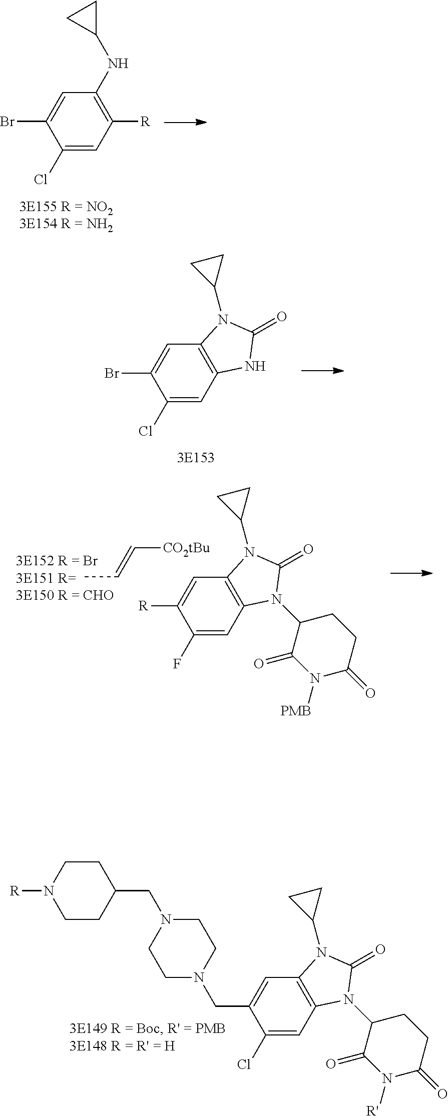

1-Bromo-2,5-difluoro-4-nitrobenzene (7.0 g) and cyclo-propyl amine (3.4 g) were stirred ON in THF (70 mL). The OL (water/EtOAc) was dried and concentrated to give Int. 3E155 5-bromo-N-cyclo-propyl-4-fluoro-2-nitro-aniline (8.0 g). This material, NH₄Cl (13.5 g), and Iron powder (7.1 g) were stirred 3 h in EtOH/water (1:1; 70 mL) at 70-80° C. and filtered. The OL (water/EtOAc) was dried and concentrated to give Int. 3E154 4-bromo-N2-cyclo-propyl-5-fluoro-benzene-1,2-diamine (5.0 g). Int. 3E154 (6.0 g) and CDI (6.0 g) were stirred ON in ACN (60 mL) at 80° C., and diluted with water to precipitate Int. 3E153 5-bromo-3-cyclo-propyl-6-fluoro-1H-benzimidazol-2-one (4.2 g). This material and KOtBu (2.8 g) were stirred 0.5 h in THF (42 mL) at 0° C. Int. 3E3 (9.5 g in 10 mL THF) was added and stirring continued 3 h at 0° C. The OL (EtOAc/water) was washed with brine, dried, and concentrated to give Int. 3E152 3-(5-bromo-3-cyclo-propyl-6-fluoro-2-oxo-benzimidazol-1-yl)-1-[(4-methoxyphenyl)methyl]-piperidine-2,6-dione (4.2 g). This material, tert-butyl acrylate (3.1 mL), Pd (OAc) 2 (0.45 g), and P (o-tol)₃ (0.51 g) were degassed in DMF/Et₃N (5.9:1, 23.4 mL) and stirred ON at 110° C. The OL (water/EtOAc) was washed with brine, dried, concentrated, and purified by FC (hexane/EtOAc 3:2) to give Int. 3E151 tert-butyl (E)-3-[3-cyclo-propyl-6-fluoro-1-[1-[(4-methoxyphenyl)-methyl]-2,6-dioxo-3-piperidyl]-2-oxo-benzimidazol-5-yl]prop-2-enoate (4.3 g). This material, NaIO₄ (4.2 g), and OsO₄ (0.4 g) were stirred ON in THF/water (3:1; 44 mL). The OL (sat. aq.

Na₂S₂O₃ and sat. aq. NaHCO₃/EtOAc) was washed with brine, dried, and concentrated to give Int. 3E150 3-cyclo-propyl-6-fluoro-1-[1-[(4-methoxyphenyl)methyl]-2,6-dioxo-3-piperidyl]-2-oxo-benzimidazole-5-carbaldehyde (2.4 g). Ints. 3E5/3E150 (0.59 g/0.47 g) were stirred ON in DCE/DIPEA (9.3:1, 5.5 mL). STAB (0.44 g) was added and stirring continued ON at 0° C. to RT. The OL (10% aq. NH₄Cl/DCM/MeOH (9:1) was washed with brine, dried, concentrated, and purified by FC (hexane/EtOAc 1:4) to give Int. 3E149 tert-butyl 4-[[4-[3-cyclo-propyl-6-fluoro-1-[1-[(4-methoxyphenyl)methyl]-2,6-dioxo-3-piperidyl]-2-oxo-benzimidazol-5-yl]methyl]-piperazin-1-yl] methyl]-piperidine-1-carboxylate (0.23 g). 0.18 g of this material was stirred 1 h in TFA/TfOH (10:1; 3.3 mL) at 0° C. and 1 h at 70° C. and concentrated to give Int. 3E148 3-(3-cyclo-propyl-6-fluoro-2-oxo-5-((4-(piperidin-4-ylmethyl)piperazin-1-yl)methyl)-2,3-dihydro-1H-benzo[d]imidazol-1-yl) piperidine-2,6-dione (0.27 g, TfOH salt).

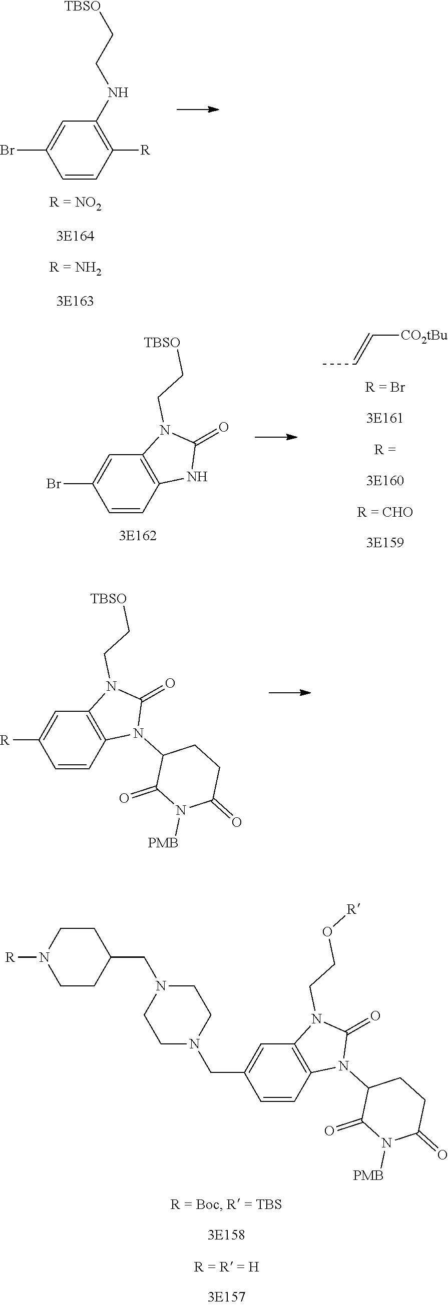

1-Bromo-3-fluoro-4-nitrobenzene (1.0 g), 2-[tert-butyl(dimethyl) silyl] oxyethanamine (0.96 g), and K₂CO₃ (1.3 g) were stirred ON in THF (5 mL) at 80° C. The OL (EtOAc/water) was dried and concentrated and combined with another batch prepared similarly from 1-bromo-3-fluoro-4-nitrobenzene (4.8 g) to give Int. 3E164 5-bromo-N-[2-[tert-butyl(dimethyl) silyl] oxyethyl]-2-nitro-aniline (8.0 g). This material, NH₄Cl (11.3 g), and iron powder (5.8 g) were stirred ON in EtOH/water (1:1; 60 mL) at 70° C. and filtered. The OL (water/EtOAc) was dried and concentrated to give Int. 3E163 4-bromo-N2-[2-[tert-butyl(dimethyl) silyl]-oxyethyl]-benzene-1,2-diamine (6.0 g). Int. 3E163 (5.5 g) and CDI (5.2 g) were stirred ON in ACN (30 mL) at 80° C., concentrated, and triturated in water to give Int. 3E162 5-bromo-3-[2-[tert-butyl-(dimethyl)-silyl]-oxyethyl]-1H-benz-imidazol-2-one (5.0 g). Int. 3E162 (4.1 g) and KOtBu (1.9 g) were stirred 0.5 h in THF (10 mL) at 0° C. Int. 3E3 (8.4 g) was added and stirring continued 4 h at 0° C. The OL (water/EtOAc) was washed with brine, dried, and concentrated to give Int.

3E161 3-[5-bromo-3-[2-[tert-butyl-(dimethyl) silyl] oxy-ethyl]-2-oxo-benzimidazol-1-yl]-1-[(4-methoxyphenyl)-methyl]piperidine-2,6-dione (2.0 g). This material, tert-butyl acrylate (1.7 g), Pd (OAc) 2 (0.23 g), and P (o-tol); (0.21 g) were degassed in DMF/Et₃N (5.9:1, 5.4 mL) and stirred ON at 110° C. The OL (water/EtOAc) was washed with brine, dried, concentrated, and purified by FC (hexane/EtOAc 7:3 to 3:2) to give Int. 3E160 tert-butyl (E)-3-[3-[2-[tert-butyl(dimethyl) silyl]-oxyethyl]-1-[1-[(4-methoxyphenyl)methyl]-2,6-dioxo-3-piperidyl]-2-oxo-benzimidazol-5-yl]prop-2-enoate (1.5 g). This material, NaIO₄ (1.5 g), and OsO₄ (6 mg) were stirred ON in THF/water (2:1; 15 mL) at 0° C. to RT. The OL (sat. aq. Na₂S₂O₃ and sat. aq. NaHCO/EtOAc) was washed with brine, dried, concentrated, and purified by FC (pentane/EtOAc 3:7 to 1:4) to give Int. 3E159 3-[2-[tert-butyl(dimethyl) silyl] oxyethyl]-1-[1-[(4-methoxy-phenyl)methyl]-2,6-dioxo-3-piperidyl]-2-oxo-benzimidazole-5-carbaldehyde (0.8 g). Ints. 3E5/3E159 (0.53 g/0.8 g) were stirred ON in DCE/DIPEA (19:1, 26 mL). STAB (0.77 g) was added and stirring continued ON at 0° C. to RT. The OL (water/DCM) was concentrated and purified by FC (DCM/MeOH 95:5 to 9:1) to give Int. 3E158 tert-butyl 4-[[4-[3-[2-[tert-butyl-(dimethyl) silyl] oxyethyl]-1-[1-[(4-methoxyphenyl)-methyl]-2,6-dioxo-3-piperidyl]-2-oxo-benzimidazol-5-yl]methyl]piperazin-1-yl]methyl]-piperidine-1-carboxylate (0.24 g). This material (0.16 g) was stirred 4 h in 1.8M HCl in dioxane (9 mL) at 0° C. to RT, concentrated, and triturated in Et₂O to give Int. 3E157 3-[3-(2-hydroxyethyl)-2-oxo-5-[4-(4-piperidylmethyl)-piperazin-1-yl]methyl] benzimidazol-1-yl]-1-[(4-methoxy-phenyl)methyl]-piperidine-2,6-dione (0.13 g, HCl salt).

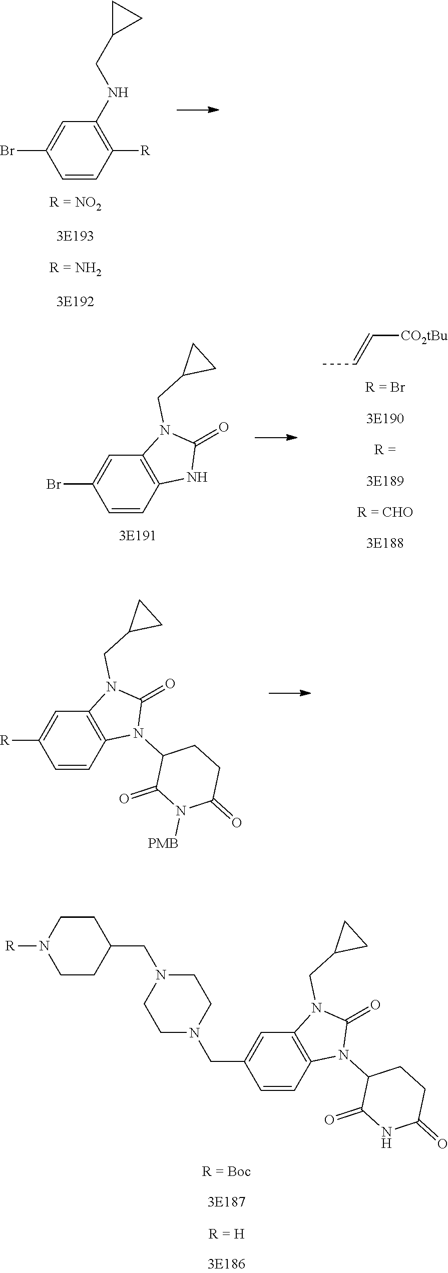

cyclo-Propylmethanamine (4.5 g) and 4-bromo-2-fluoro-1-nitrobenzene (7.0 g) were stirred in THF (50 mL) at 0° C. The OL (water/EtOAc) was washed with brine, dried, and concentrated to give Int. 3E193 5-bromo-N-(cyclo-propylmethyl)-2-nitro-aniline (8.0 g). This material, NH₄Cl (15.0 g), and iron powder (7.8 g) were stirred 3 h in EtOH/water (2:1; 60 mL) at 80° C. and filtered. The OL (water/EtOAc) was dried and concentrated to give Int. 3E194 4-bromo-N2-(cyclo-propylmethyl)benzene-1,2-diamine (6.5 g). Int. 3E194 (5.0 g) and CDI (4.3 g) were stirred 3 h in ACN (30 mL) at 80° C. The OL (water/EtOAc) was washed with brine, dried, concentrated, and triturated in Et₂O to give Int. 3E191 5-bromo-3-(cyclo-propylmethyl)-1H-benzimidazol-2-one (4.7 g). Int. 3E191 (2.5 g) and NaH (60% oil disp., 2.6 g) were stirred 0.5 h in THF (25 mL). 3-Bromo-piperidine-2,6-dione (6.3 g) was added and stirring continued ON at 65° C. The OL (water/EtOAc) was washed with brine, dried, concentrated, and purified by FC (pentane/EtOAc 7:3 to 1:1) to Int. 3E190 3-[5-bromo-3-(cyclo-propylmethyl)-2-oxo-benzimidazol-1-yl]piperidine-2,6-dione (1.5 g). This material, tert-butyl acrylate (2.0 g), Pd (OAc) 2 (0.27 g), and P (o-tol)₃ (0.24 g) were degassed in DMF/Et₃N (4.5:1, 12.2 mL) and stirred ON at 120° C. The OL (water/EtOAc) was washed with brine, dried, concentrated, and purified by FC (pentane/EtOAc 7:3 to 3:2) to give Int. 3E189 tert-butyl (E)-3-[3-(cyclo-propylmethyl)-1-(2,6-dioxo-3-piperidyl)-2-oxo-benzimidazol-5-yl]prop-2-enoate (1.0 g). Int. 3E189 (1.4 g), NaIO₄ (1.8 g), and OsO₄ (17 mg) were stirred ON in THF/water (5:1; 18 mL) at 0° C. to RT. The OL (sat. aq. Na₂S₂O₃ and sat. aq. NaHCO₃/EtOAc) was washed with brine, dried, concentrated, and purified by FC (pentane/EtOAc 4:1) to give Int. 3E188 3-(cyclo-propylmethyl)-1-(2,6-dioxo-3-piperidyl)-2-oxo-benzimidazole-5-carbaldehyde (0.7 g). Ints. 3E5/3E188 (0.35 g/0.25 g) were stirred ON in DCE/DIPEA (10:1, 5.5 mL). STAB (0.23 g) was added and stirring continued ON at 0° C. to RT. The OL (water/DCM) was concentrated and purified by FC (DCM/MeOH 95:5 to 9:1) to give Int. 3E187 tert-butyl 4-[[4-[3-(cyclo-propylmethyl)-1-(2,6-dioxo-3-piperidyl)-2-oxo-benzimidazol-5-yl]methyl]piperazin-1-yl]methyl]-piperidine-1-carboxylate (0.14 g). Int. 3E187 (0.13 g) was stirred 5 h in DCM/4M HCl in dioxane (5:1.3; 6.3 mL) at 0° C. to RT and concentrated to give Int. 3E186 3-(3-(cyclo-propylmethyl)-2-oxo-5-((4-(piperidin-4-ylmethyl)-piperazin-1-yl)methyl)-2,3-dihydro-1H-benzo[d]imidazol-1-yl) piperidine-2,6-dione (0.12 g, HCl salt).

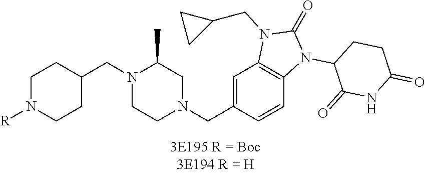

Ints. 3E74/3E188 (0.27 g/0.20 g) were stirred ON in DCE/DIPEA (9.4:1, 2.5 mL). STAB (0.39 g) was added and stirring continued 36 h at 0° C. to RT. The OL (water/DCM) was washed with brine, dried, concentrated, and purified by FC (DCM/MeOH 95:5 to 9:1) to give Int. 3E195 tert-butyl 4-(((2S)-4-((3-(cyclo-propylmethyl)-1-(2,6-dioxopiperidin-3-yl)-2-oxo-2,3-dihydro-1H-benzo[d]-imidazol-5-yl)methyl)-2-methyl-piperazin-1-yl)-methyl)-piperidine-1-carboxylate (0.20 g). This material was stirred 3 h in 0.5M HCl in dioxane (2.3 mL) at 0° C. to RT and concentrated to give Int. 3E194 3-(3-(cyclo-propylmethyl)-5-(((S)-3-methyl-4-(piperidin-4-ylmethyl)piperazin-1-yl)methyl)-2-oxo-2,3-dihydro-1H-benzo[d] imidazol-1-yl) piperidine-2,6-dione (0.15 g, HCl salt).

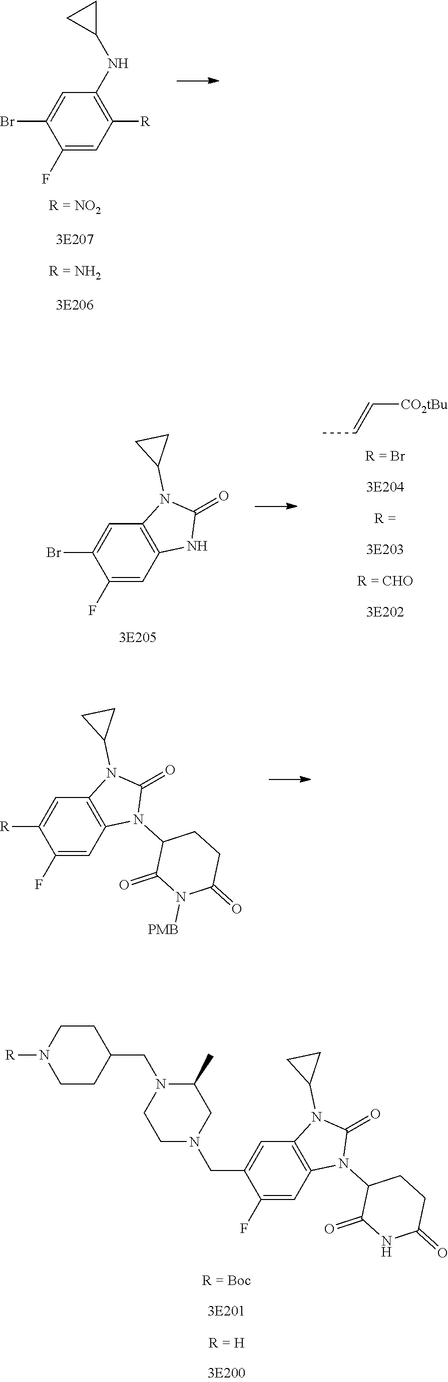

cyclo-Propylmethanamine (3.4 g) and 1-bromo-2,5-difluoro-4-nitrobenzene (7.0 g) were stirred ON in THF (70 mL). The OL (water/EtOAc) was dried and concentrated to give Int. 3E207 5-bromo-N-cyclo-propyl-4-fluoro-2-nitro-aniline (8.0 g). 7.0 g of this material, NH₄Cl (13.6 g), and iron powder (7.1 g) were stirred 3 h in EtOH/water (1:1; 70 mL) at 70-80° C. and filtered. The OL (water/EtOAc) was dried and concentrated to give Int. 3E206 4-bromo-N2-cyclo-propyl-5-fluoro-benzene-1,2-diamine (5.0 g). Int. 3E206 (6.0 g) and CDI (5.9 g) were stirred ON in ACN (60 mL) at 80° C., concentrated, and triturated in water to give Int. 3E205 5-bromo-3-cyclo-propyl-6-fluoro-1H-benzimidazol-2-one (4.2 g). Int. 3E205 (6.0 g) and KOtBu (4.0 g) were stirred 0.5 h in THF (60 mL). Int. 3A8 (13.5 g) was added and stirring continued 3 h at 0° C. The OL (aq. NH₄Cl water/DCM/MeOH (9:1)) was washed with brine, dried, and concentrated to give Int. 3E204 3-(5-bromo-3-cyclo-propyl-6-fluoro-2-oxo-benzimidazol-1-yl)-1-[(4-methoxy-phenyl)methyl]-piperidine-2,6-dione (7.1 g). 6.1 g of this material, tert-butyl acrylate (5.3 mL), Pd (OAc) 2 (0.82 g), and P (o-tol)₃ (0.74 g) were degassed in DMF/Et₃N (10:1, 55 mL) and stirred ON at 110° C. . . . The OL (water/EtOAc) was washed with brine, dried, concentrated, and purified by FC (pentane/EtOAc 3:2) to give Int. 3E203 tert-butyl (E)-3-[3-cyclo-propyl-6-fluoro-1-[1-[(4-methoxyphenyl)methyl]-2,6-dioxo-3-piperidyl]-2-oxo-benzimidazol-5-yl]prop-2-enoate (6.0 g). This material, NaIO₄ (5.8 g), and OsO₄ (0.55 mg) were stirred ON in THF/water (3:1; 60 mL). The OL (water/EtOAc) was washed with brine, dried, concentrated, and purified by FC (hexane/EtOAc 3:2) to give Int. 3E202 3-cyclo-propyl-6-fluoro-1-[1-[(4-methoxyphenyl)-methyl]-2,6-dioxo-3-piperidyl]-2-oxo-benzimidazole-5-carbaldehyde (3.1 g). Ints. 3E5/3E202 (0.99 g/1.5 g) were stirred ON in DCE/DIPEA (5.6:1, 11.8 mL). STAB (1.4 g in DMSO (5 mL)) was added at 0° C. and stirring continued ON at 0° C. to 60° C. The OL (aq. NH₄Cl/EtOAc) was concentrated and purified by FC (hexane/EtOAc 1:4) to give Int. 3E201 tert-butyl 4-[[(2S)-4-[3-cyclo-propyl-6-fluoro-1-[1-[(4-methoxyphenyl)-methyl]-2,6-dioxo-3-piperidyl]-2-oxo-benz-imidazol-5-yl]methyl]-2-methyl-piperazin-1-yl]methyl]piperidine-1-carboxylate (1.2 g). This material was stirred 2 h in TFA/TfOH (9:1; 10 mL) at 70° C. and concentrated to give Int. 3E200 3-(3-cyclo-propyl-6-fluoro-5-(((S)-3-methyl-4-(piperidin-4-ylmethyl)piperazin-1-yl)methyl)-2-oxo-2,3-dihydro-1H-benzo[d]imidazol-1-yl) piperidine-2,6-dione (1.5 g, TfOH salt).

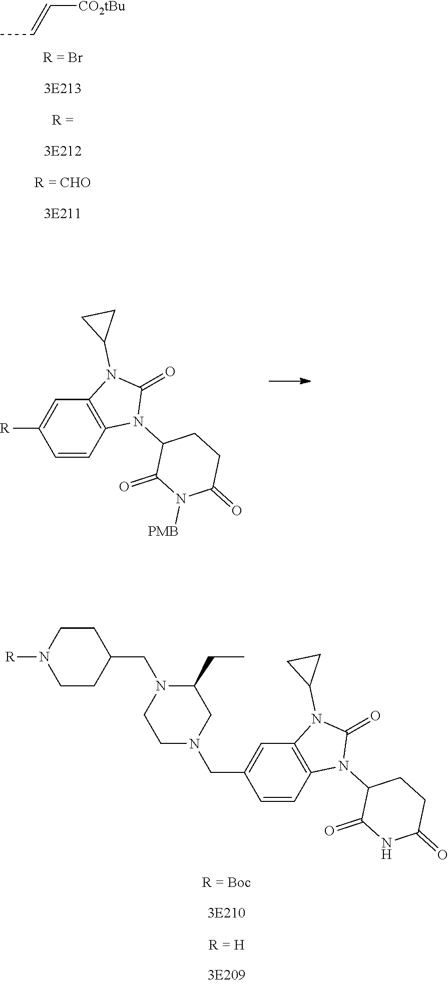

5-Bromo-3-cyclo-propyl-1H-benzimidazol-2-one (3.0 g) and NaH (60% oil disp. 7.9 g) were stirred 0.3 h in THF (70 mL) at 0° C. 3-Bromopiperidine-2,6-dione (19.0 g) was added and stirring continued ON at 0-60° C. The OL (water/EtOAc) was washed with brine, dried, concentrated, and triturated in hexane to give Int. 3E213 3-(5-bromo-3-cyclo-propyl-2-oxo-benzimidazol-1-yl) piperidine-2,6-dione (5.2 g). Int. 3E213 (6.1 g), tert-butyl acrylate (2.6 g), Pd (OAc) 2 (0.44 g), and P (o-tol)₃ (0.50 g) were degassed in DMF/Et₃N (17.6:1, 63.4 mL) and stirred ON at 110° C. and filtered. The OL (water/EtOAc). was washed with brine, dried, concentrated, and triturated in pentane to give Int. 3E212 tert-butyl (E)-3-[3-cyclo-propyl-1-(2,6-dioxo-3-piperidyl)-2-oxo-benz-imidazol-5-yl]prop-2-enoate (2.9 g). 1.4 g of this material, NaIO₄ (1.8 g), and OsO₄ (0.43 g) were stirred ON in THF/water (4.7:1, 17 mL). The OL (water/EtOAc) was washed with brine, dried, concentrated, and purified by FC (pentane/EtOAc 1:1 to 2:3) to give Int. 3E211 3-cyclo-propyl-1-(2,6-dioxo-3-piperidyl)-2-oxo-benzimidazole-5-carbaldehyde (0.80 g). Ints. 3E101/3E211 (1.4 g/0.55 g) and ZnCl₂ were stirred ON in DCE (6 mL). NaCNBH₃ (0.22 g) was added and stirring continued ON at 0° C. to RT. The OL (water/EtOAc) was washed with brine, dried, concentrated, and purified by FC (DCM/MeOH 1:0 to 4:1) to give Int. 3E210 tert-butyl 4-[[(2S)-4-[3-cyclo-propyl-1-(2,6-dioxo-3-piperidyl)-2-oxo-benzimidazol-5-yl]methyl]-2-ethyl-piperazin-1-yl]methyl]-piperidine-1-carboxylate (0.80 g). A solution of 0.75 g of this material in DCM/4M HCl in dioxane (5:1, 6 mL) was stirred ON at 0° C. to RT and concentrated to give Int. 3E209 3-[3-cyclo-propyl-5-[[(3S)-3-ethyl-4-(4-piperidylmethyl)piperazin-1-yl]methyl]-2-oxo-benzimidazol-1-yl]-piperidine-2,6-dione (0.65 g, HCl salt).

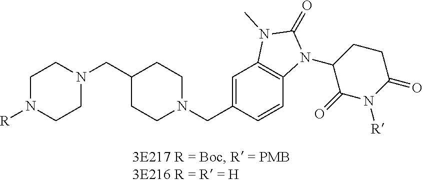

Ints. 3E3/3E218 (50 mg/42 mg) were stirred 0.25 h in DCE (2 mL) and a few drops of DMF. STAB (36 mg) was added and stirring continued 120 h. STAB (77 mg) and DIPEA (86 microL) were added and stirring continued 2 h at 70° C. The OL (water/DCM) was washed with brine, dried, concentrated, and purified by FC (DCM/MeOH 1:0 to 4:1) to give Int. 3E217 tert-butyl 4-[[1-[1-[1-[(4-methoxyphenyl)methyl]-2,6-dioxo-3-piperidyl]-3-methyl-2-oxo-benzimidazol-4-yl]methyl]-4-piperidyl]-methyl]piperazine-1-carboxylate (33 mg). This material was stirred 4 h in TFA/TfOH (2:0.05; 2.05 mL) and ON at 50° C. and concentrated to give Int. 3E216 3-(3-methyl-2-oxo-5-((4-(piperazin-1-ylmethyl)-piperidin-1-yl)methyl)-2,3-dihydro-1H-benzo[d]-imidazol-1-yl) piperidine-2,6-dione (22 mg, TfOH salt).

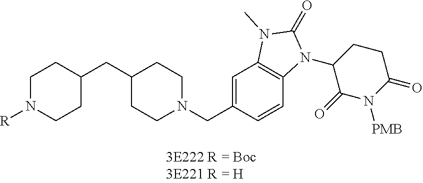

Int. 3E3 (50 mg) tert-butyl 4-(piperidin-4-ylmethyl)piperidine-1-carboxylate (47 mg, HCl salt) were stirred 0.3 h in DCE/AcOH/DIPEA (28.6:1:6.1, 2.5 mL) at 70° C. STAB (0.13 g) was added and stirring continued 3 h. The OL (water/DCM) was washed with brine, dried, concentrated, and purified by FC (DCM/MeOH 1:0 to 4:1) to give Int. 3E222 tert-butyl 4-[1-[1-[1-[(4-methoxyphenyl)methyl]-2,6-dioxo-3-piperidyl]-3-methyl-2-oxo-benz-imidazol-4-yl]methyl]-4-piperidyl]-methyl]piperazine-1-carboxylate (57 mg). A solution of this material in DCM/TFA (4:1; 2.5 mL) was stirred 0.5 h and concentrated to give Int. 3E221 3-[3-methyl-2-oxo-4-[[4-(4-piperidylmethyl)piperazin-1-yl]-methyl] benzimidazol-1-yl]piperidine-2,6-dione (58 mg, TFA salt).

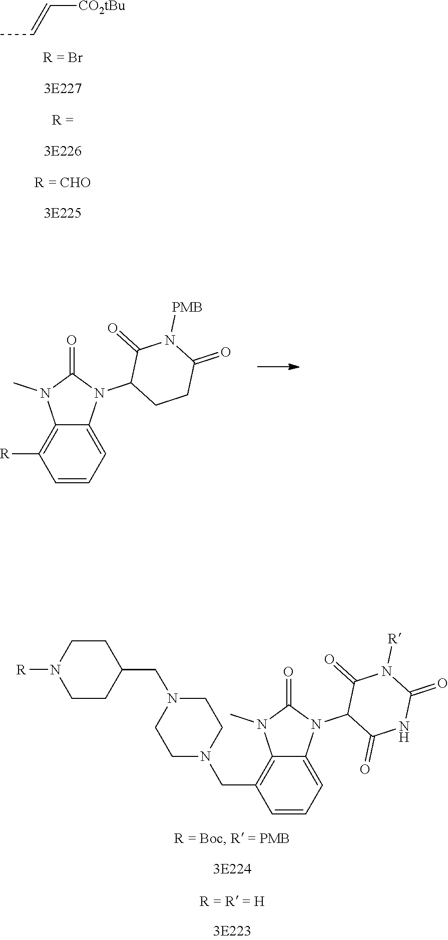

7-Bromo-1-methyl-1,3-dihydro-2H-benzo[d]imidazol-2-one (0.25 g) and KOtBu (0.16 g) were stirred 0.25 h in THF (3 mL) at 0° C. Int. 3A8 (0.67 g) was added and stirring continued 1 h. The residue after concentration was purified by FC (pentane/EtOAc 1:0 to 7:3) to give Int. 3E227 3-(4-bromo-3-methyl-2-oxo-benzimidazol-1-yl)-1-[(4-methoxy-phenyl)methyl]-piperidine-2,6-dione (0.15 g). Int. 3E227 (0.4 g), tert-butyl acrylate (0.32 mL), Pd (OAc) 2 (20 mg), and P (o-tol); (53 mg) were degassed in DMF/Et₃N (16.6:1, 5.4 mL) and stirred ON at 120° C. The OL (water/EtOAc) was washed with brine, dried, concentrated, and purified by FC (heptane/EtOAc 1:1 to 0:1) to give Int. 3E226 tert-butyl (E)-3-[1-[1-[(4-methoxyphenyl)-methyl]-2,6-dioxo-3-piperidyl]-3-methyl-2-oxo-benzimidazol-4-yl]prop-2-enoate (0.44 g). This material and NaIO₄ (0.47 g) were stirred ON in THF/water (2:1; 3 mL) and 2.5% OsO₄ in tBuOH (0.11 mL). The OL (2MeTHF/sat. aq. NaHCO₃) was washed with 5% aq. Na₂S₂O₃ and brine, dried, concentrated, and triturated in DCM/heptane to give Int. 3E225 1-(1-(4-methoxy-benzyl)-2,6-dioxopiperidin-3-yl)-3-methyl-2-oxo-2,3-dihydro-1H-benzo[d]imidazole-4-carbaldehyde (0.34 g). Ints. 3E5/3E225(42 mg/50 mg) were stirred 0.3 h in DCE/AcOH/DIPEA (28.6:1:6.1, 2.1 mL) at 70° C. STAB (0.13 g) was added and stirring continued 1 h at 70° C. The OL (water/DCM) was washed with brine, dried, concentrated, and purified by FC (DCM/MeOH 1:0 to 4:1) to give Int. 3E224 tert-butyl 4-[1-[1-[1-[(4-methoxyphenyl)-methyl]-2,6-dioxo-3-piperidyl]-3-methyl-2-oxo-benzimidazol-4-yl] methyl]-4-piperidyl]-methyl]piperazine-1-carboxylate (74 mg). A solution of this material in TFA/TfOH (2:0.1; 2.1 mL) was stirred 4 h at 70° C. and ON at 50° C. and concentrated to give Int. 3E223 3-(3-methyl-2-oxo-4-((4-(piperidin-4-ylmethyl)-piperazin-1-yl)methyl)-2,3-dihydro-1H-benzo[d] imidazol-1-yl) piperidine-2,6-dione (50 mg, TfOH salt).

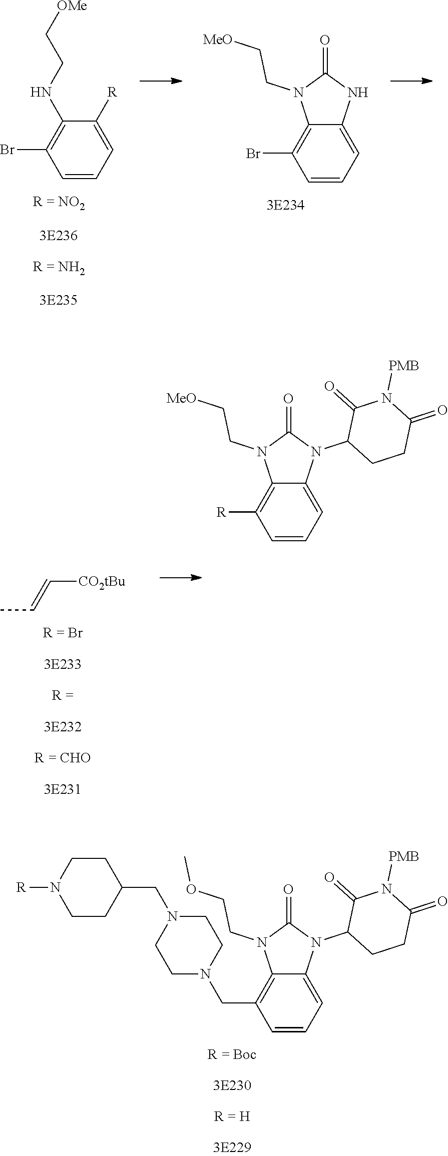

1-Bromo-2-fluoro-3-nitro-benzene (5.0 g) and 2-methoxyethan-1-amine (1.9 g) were stirred ON in MeOH (50 mL) at 0° C. to RT. The OL (water/EtOAc) was washed with brine, dried, and concentrated to give Int. 3E236 2-bromo-N-(2-methoxyethyl)-6-nitro-aniline (6.0 g). 3.8 g of this material and iron powder (3.9 g) were stirred 2 h in AcOH (38 mL) at 40° C. and filtered. The OL (aq. NaHCO₃/EtOAc) was washed with brine, dried, concentrated, and purified by FC (pentane/EtOAc 9:1 to 4:1) to give Int. 3E235 3-bromo-N2-(2-methoxyethyl)benzene-1,2-diamine (2.5 g). This material and CDI (2.5 g) were stirred ON in ACN (25 mL) at 80° C. and concentrated. The residue was triturated in water to give Int. 3E234 4-bromo-3-(2-methoxy-ethyl)-1H-benzimidazol-2-one (2.5 g). 0.7 g of this material and KOtBu (0.32 g) were stirred 0.5 h in THF (14 mL) at −10° C. to 0° C. Int. 3A8 (1.48 g) was added as a solution in THF (20 mL) at −10° C. and stirring was continued 1 h at 0° C. The OL (sat. aq. NH₄Cl/EtOAc) was washed with brine, dried, concentrated, and purified by FC (hexane/EtOAc 9:1 to 4:1) to give Int. 3E233 3-[4-bromo-3-(2-methoxyethyl)-2-oxo-benzimidazol-1-yl]-1-[(4-methoxy-phenyl)-methyl]-piperidine-2,6-dione (0.61 g). 0.4 g of this material, tert-butyl acrylate (0.29 mL), Pd (OAc) 2 (18 mg), and P (o-tol)₃ (48 mg) were degassed in DMF/Et₃N (16.7:1, 5.3 mL) and stirred ON at 120° C. The OL (water/EtOAc) was washed with brine, dried, concentrated, and purified by FC (heptane/EtOAc 1:0 to 0:1) to give Int.

3E232 tert-butyl (E)-3-(1-(1-(4-methoxybenzyl)-2,6-dioxo-piperidin-3-yl)-3-(2-methoxyethyl)-2-oxo-2,3-dihydro-1H-benzo[d]imidazol-4-yl)-acrylate (0.41 g). 0.39 g of this material and NaIO₄ (0.38 g), and 2.5% OsO₄ in tBuOH (89 microL) were stirred ON in THF/water (2:1; 9 mL). The OL (sat. aq. Na₂S₂O₃ and sat. aq. NaHCO/EtOAc) was washed with brine, dried, concentrated, and purified by FC (EtOAc/heptane 0:1 to 1:0) to give Int. 3E231 3-(2-methoxy-ethyl)-1-[1-[(4-methoxyphenyl)-methyl]-2,6-dioxo-3-piperidyl]-2-oxo-benz-imidazole-4-carbaldehyde (0.26 g). Ints. 3E5/3E231 (0.11 g/0.12 g) were stirred 0.5 h in DCE/DIPEA (87:1, 4.05) at 70° C. STAB (0.28 g) was added and stirring continued 4 h at 70° C. The OL (water/DCM) was washed with brine, dried, concentrated, and purified by FC (DCM/MeOH 1:0 to 4:1) to give Int. 3E230 tert-butyl 4-((4-((1-(1-(4-methoxybenzyl)-2,6-dioxopiperidin-3-yl)-3-(2-methoxyethyl)-2-oxo-2,3-dihydro-1H-benzo[d]-imidazol-4-yl)methyl)piperazin-1-yl)methyl)piperidine-1-carboxylate (0.15 g). This material was stirred 1 h in DCM/TFA (3:2; 5 mL) and concentrated to give Int. 3E229 1-(4-methoxybenzyl)-3-(3-(2-methoxy-ethyl)-2-oxo-4-((4-(piperidin-4-ylmethyl)piperazin-1-yl)methyl)-2,3-dihydro-1H-benzo[d]-imidazol-1-yl)-piperidine-2,6-dione (0.11 g, TFA salt).

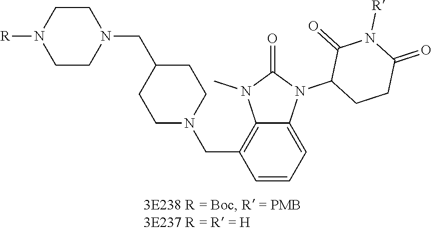

Ints. 3E225/3E218 (50 mg/42 mg) were stirred 0.3 h in DCE/AcOH/DIPEA (28.6:1:6.4, 2.0.5 mL) at 70° C. STAB (0.13 g) was added and stirring continued 1 h at 70° C. The OL (water/DCM) was washed with brine, dried, concentrated, and purified by FC (DCM/MeOH 1:0 to 4:1) to give Int. 3E238 tert-butyl 4-[1-[1-[1-[(4-methoxyphenyl)methyl]-2,6-dioxo-3-piperidyl]-3-methyl-2-oxo-benzimidazol-4-yl]methyl]-4-piperidyl]-methyl]piperazine-1-carboxylate (77 mg). This material was stirred 4 h in TFA/TfOH (2:0.1; 2.1 mL) at 70° C. and concentrated to give Int. 3E237 3-[3-methyl-2-oxo-4-[[4-(piperazin-1-ylmethyl)-1-piperidyl]methyl]-benzimidazol-1-yl]piperidine-2,6-dione (52 mg, TfOH salt).

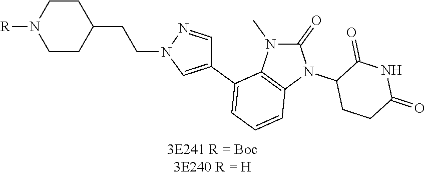

Ints. 3E242/3A6 (55 mg/35 mg), and PdDPPFCl₂-DCM (9 mg) were degassed in DMF/10% aq. Na₂CO₃ (5.6:1, 1.5 mL) and stirred 1.25 h at 100° C. The OL (water/EtOAc) was washed with brine, dried, concentrated, and purified by FC (heptane/EtOAc 3:2 to 0:1) to give Int. 3E241 tert-butyl 4-(2-(4-(1-(2,6-dioxopiperidin-3-yl)-3-methyl-2-oxo-2,3-dihydro-1H-benzo[d]imidazol-4-yl)-1H-pyrazol-1-yl)-ethyl)-piperidine-1-carboxylate (18 mg). A solution of Int. 3E241 (16 mg) in MeOH/4M HCl in dioxane (1:1; 0.4 mL) was stirred 0.75 h and concentrated to give Int. 3E240 3-(3-methyl-2-oxo-4-(1-(2-(piperidin-4-yl)-ethyl)-1H-pyrazol-4-yl)-2,3-dihydro-1H-benzo[d]imidazol-1-yl)-piperidine-2,6-dione (17 mg, HCl salt).

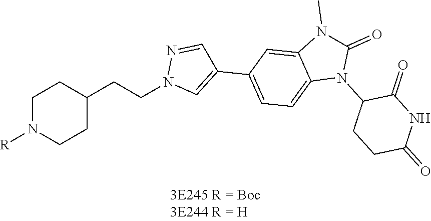

Ints. 3E242/3B3 (55 mg/35 mg), and PdDPPFCl₂-DCM (9 mg) were degassed in DMF/10% aq. Na₂CO₃ (5.7:1, 1.5 mL) and stirred 1.25 h at 100° C. The OL (water/EtOAc) was washed with brine, dried, concentrated, and purified by FC (heptane/EtOAc 3:2 to 0:1) to give Int. 3E245 tert-butyl 4-(2-(4-(1-(2,6-dioxopiperidin-3-yl)-3-methyl-2-oxo-2,3-dihydro-1H-benzo[d]imidazol-5-yl)-1H-pyrazol-1-yl)ethyl)-piperidine-1-carboxylate (27 mg). 25 mg of this material was stirred 0.75 h in MeOH/4M HCl in dioxane (1:1; 0.6 mL) and concentrated to give Int. 3E244 3-[3-methyl-2-oxo-5-[1-[2-(4-piperidyl)ethyl]pyrazol-4-yl] benzimidazol-1-yl]-piperidine-2,6-dione (26 mg, HCl salt).

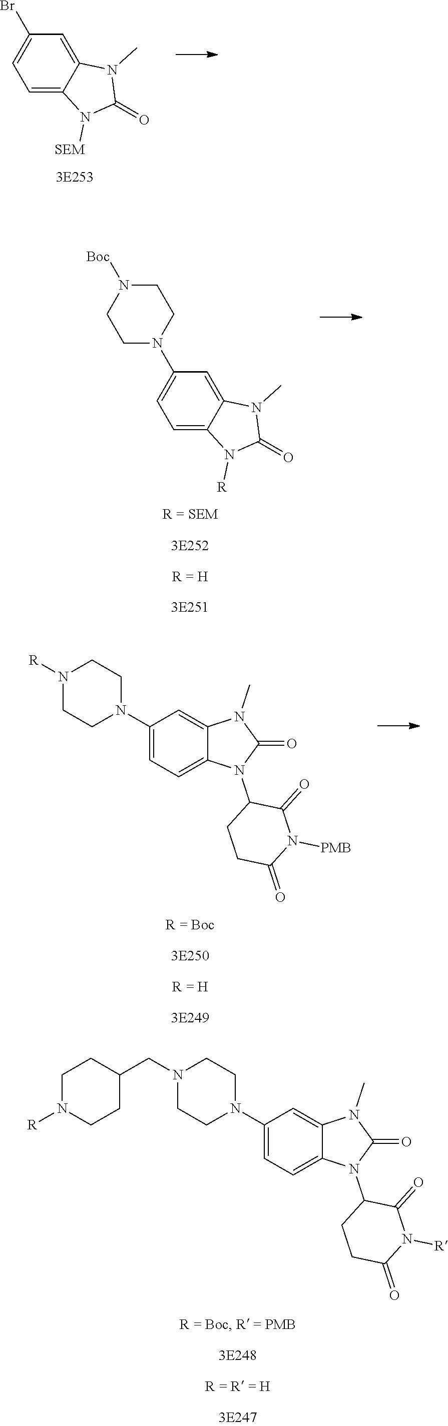

6-Bromo-1-methyl-1,3-dihydro-2H-benzo[d]imidazol-2-one (5.0 g) and NaH (1.32 g) were stirred 0.5 h in DMF (50 mL) at 0° C. SEM-Cl (4.0 g) was added and stirring continued ON at 0° C. to RT. The OL (water/EtOAc) was washed with brine, dried, concentrated, and purified by FC (hexane/EtOAc 3:2) to give Int. 3E253 5-bromo-3-methyl-1-((2-(trimethylsilyl) ethoxy)methyl)-1,3-dihydro-2H-benzo[d]imidazol-2-one (6.5 g). This material, tert-butyl piperazine-1-carboxylate (5.1 g), Pd₂dba₃ (1.7 g), RuPhos (0.85 g), and NaOtBu (3.5 g) were degassed in dioxane (65 mL) and stirred ON at 90° C. Filtration, concentration, and purification by FC (EtOAc/hexane 1:1) to give Int. 3E252 tert-butyl 4-(3-methyl-2-oxo-1-((2-(trimethylsilyl) ethoxy)-methyl)-2,3-dihydro-1H-benzo[d]-imidazol-5-yl) piperazine-1-carboxylate (8.0 g). 2.0 g of this material and TBAF (9.6 g) were stirred ON in THF (20 mL) at 80° C. The OL (water/EtOAc) was dried, concentrated, and purified by FC (hexane/EtOAc 3:7) to give Int. 3E251 tert-butyl 4-(3-methyl-2-oxo-2,3-dihydro-1H-benzo[d]-imidazol-5-yl) piperazine-1-carboxylate (1.1 g). This material and KOtBu (0.48 g) were stirred 0.5 h in THF (20 mL) at 0° C. Int. 3A8 (2.5 g) was added and stirring continued 2 h at 0° C. to RT. The OL (sat. aq. NH₄Cl/EtOAc) was dried, concentrated, and purified by FC (pentane/EtOAc 1:0 to 3:7) to give Int. 3E250 tert-butyl 4-[1-[1-[(4-methoxy-phenyl)methyl]-2,6-dioxo-3-piperidyl]-3-methyl-2-oxo-benzimidazol-5-yl]piperazine-1-carboxylate (0.9 g). Int. 3E250 (2.7 g) was stirred 2 h in 1.5M HCl in dioxane (40 mL) at 0° C. and concentrated to give Int. 3E249 1-[(4-methoxy-phenyl)-methyl]-3-(3-methyl-2-oxo-5-piperazin-1-yl-benzimidazol-1-yl) piperidine-2,6-dione (1.8 g, HCl salt). 0.40 g of this material and tert-butyl 4-formyl-piperidine-1-carboxylate (0.22 g) were stirred 3 h in DMSO/DIPEA (13.3:1, 8.6 mL). STAB (0.55 g) was added at 0° C. and stirring continued 72 h at 0° C. to RT. The mixture was combined with another batch prepared from 1.0 g Int. 3E249. The OL (water/EtOAc) was washed with brine, dried, concentrated, and purified by FC (DCM/MeOH 95:5) to give on Int. 3E248 tert-butyl 4-[[4-[1-[1-[(4-methoxyphenyl)-methyl]-2,6-dioxo-3-piperidyl]-3-methyl-2-oxo-benzi-midazol-5-yl]-piperazin-1-yl] methyl]-piperidine-1-carboxylate (1.1 g). 0.8 g of this material was stirred 2 h in TFA/TfOH (2:1, 15 mL) at 0° C. to 80° C. and concentrated. The residue was triturated in Et₂O and HPLC-purified to give Int. 3E247 3-[3-methyl-2-oxo-5-[4-(4-piperidylmethyl)-piperazin-1-yl] benzimidazol-1-yl]-piperidine-2,6-dione (0.21 g, TfOH salt).

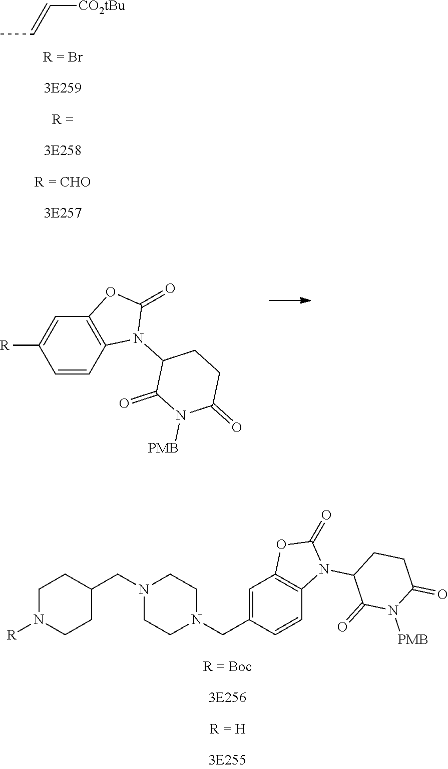

6-Bromo-3H-1,3-benzoxazol-2-one (2.0 g) and KOtBu (1.57 g) were stirred 0.5 h in THF (35 mL) at 0° C. Int. 3A8 (6.7 g) was added and stirring continued 3 h at 0° C. The OL (water/EtOAc) was washed with brine, dried, concentrated, and purified by FC (pentane/EtOAc 5:1 to 4:1) to give Int. 3E259 3-(6-bromo-2-oxo-1,3-benzoxazol-3-yl)-1-[(4-methoxyphenyl)methyl]piperidine-2,6-dione (4.1 g). 2.0 g of this material, tert-butyl acrylate (1.6 mL), Pd (OAc) 2 (0.15 g), and P (o-tol)₃ (0.27 g) were degassed in DMF/Et₃N (10:1, 21.9 mL) and stirred ON at 110° C. The OL (water/EtOAc) was washed with brine, dried, concentrated, and purified by FC (heptane/EtOAc 5:1 to 4:1) to give Int. 3E58 tert-butyl (E)-3-[3-[1-[(4-methoxyphenyl)methyl]-2,6-dioxo-3-piperidyl]-2-oxo-1,3-benzoxazol-6-yl] prop-2-enoate (1.5 g). This material, OsO₄ (0.27 g), and NaIO₄ (1.1 g) were stirred ON in THF/water (2:1; 18 mL). The OL (water/EtOAc) was washed with sat. aq. NaHCO₃ and brine, dried, concentrated, and purified by FC (pentane/EtOAc 1:1 to 2:3) to give Int. 3E257 3-[1-[(4-methoxyphenyl)methyl]-2,6-dioxo-3-piperidyl]-2-oxo-1,3-benzoxazole-6-carbaldehyde (0.6 g). Ints. 3E5/3E257 (0.41 g/0.30 g) were stirred 2 h in DCE/DIPEA (40:1, 15.4 mL) at 0° C. STAB (0.3 g) was added and stirring continued ON at 0° C. to RT. Int. 3E5 (0.41 g) and DIPEA (0.3 mL) were added and stirring continued at 2 h. STAB (0.30 g) was added and stirring continued ON. The OL (water/EtOAc) was washed with brine, dried, concentrated, and purified by FC (pentane/EtOAc 1:1 and 2:3) to give 3E256 tert-butyl 4-((4-((3-(1-(4-methoxy-benzyl)-2,6-dioxopiperidin-3-yl)-2-oxo-2,3-dihydro-benzo[d]oxazol-6-yl)methyl)-piperazin-1-yl)methyl)piperidine-1-carboxylate (0.6 g). A solution of this material in 1.5M HCl in dioxane (16 mL) was stirred 3 h at 0° C. to RT and concentrated to give Int. 3E255 1-(4-methoxybenzyl)-3-(2-oxo-6-((4-(piperidin-4-yl-methyl)-piperazin-1-yl)-methyl)benzo[d] oxazol-3 (2H)-yl)-piperidine-2,6-dione (0.48 g, HCl salt).

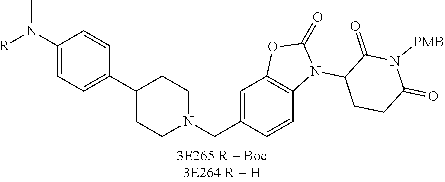

Ints. 3E257/3E266 (0.32 g/0.42 g) were stirred 2 h in DCE/DIPEA (25:1, 10.4 mL) at 0° C. to RT. STAB (0.33 g) was added and stirring continued ON at 0° C. to RT. The OL (water/EtOAc) was washed with brine, dried, concentrated, and purified by FC (pentane/EtOAc 1:1 to 2:3) to give Int. 3E265 tert-butyl N-[4-[1-[[3-[1-[(4-methoxyphenyl)methyl]-2,6-dioxo-3-piperidyl]-2-oxo-1,3-benzoxazol-6-yl] methyl]-4-piperidyl]-phenyl]-N-methyl-carbamate (0.43 g). A solution of 0.13 g of this material in 1.3M HCl in dioxane (4.5 mL) was stirred 3 h at 0° C. to RT and concentrated to give Int. 3E264 1-[(4-methoxy-phenyl)methyl]-3-[6-[[4-[4-(methyl-amino)phenyl]-1-piperidyl]-methyl]-2-oxo-1,3-benzoxazol-3-yl]-piperidine-2,6-dione (80 mg, HCl salt).

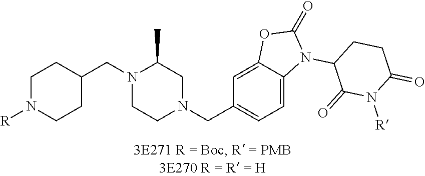

Ints. 3E74/3E257 (0.28 g/0.25 g) were stirred ON in DCE/DIPEA (24:1, 8.3 mL). STAB (0.40 g) was added and stirring continued ON. The OL (water/DCM) was dried, concentrated, and purified by FC (pentane/EtOAc 1:1) to give Int. 3E271 tert-butyl 4-[[(2S)-4-[[3-[1-[(4-methoxyphenyl)methyl]-2,6-dioxo-3-piperidyl]-2-oxo-1,3-benz-oxazol-6-yl]methyl]-2-methyl-piperazin-1-yl]methyl]piperidine-1-carboxylate (0.30 g). A solution of this material in TFA/TfOH (10:1; 3.3 mL) was stirred 1 h at 80° C. and concentrated to give Int. 3E270 3-[6-[[(3S)-3-methyl-4-(4-piperidylmethyl)piperazin-1-yl]methyl]-2-oxo-1,3-benzoxazol-3-yl]piperidine-2,6-dione (0.25 g, TfOH salt).

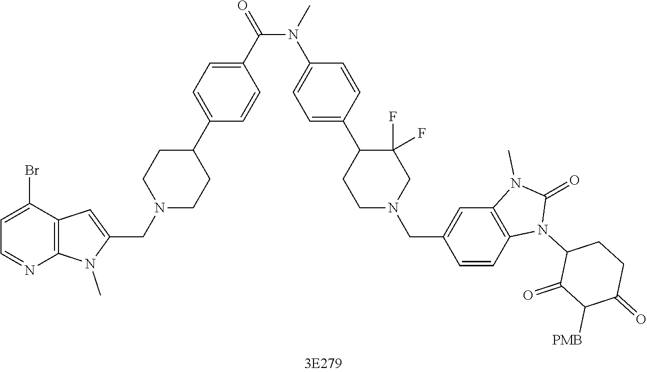

Ints. 3E3/3E280 (30 mg/52 mg) and 4 Å MS were stirred 0.5 h in DCE/DIPEA (100:1, 1.4 mL) at 70° C. STAB (78 mg) was added and stirring continued 3 h. The OL (water/DCM) was washed with brine, dried, concentrated, and purified by FC (DCM/MeOH 1:0 to 9:1) to give Int. 3E279 4-[1-[(4-bromo-1-methyl-pyrrolo-[2,3-b]pyridin-2-yl)methyl]-4-piperidyl]-N-[4-[3,3-difluoro-1-[1-[1-[(4-methoxyphenyl)-methyl]-2,6-dioxo-3-piperidyl]-3-methyl-2-oxo-benzimidazol-5-yl]methyl]-4-piperidyl]-phenyl]-N-methyl-benzamide (49 mg).

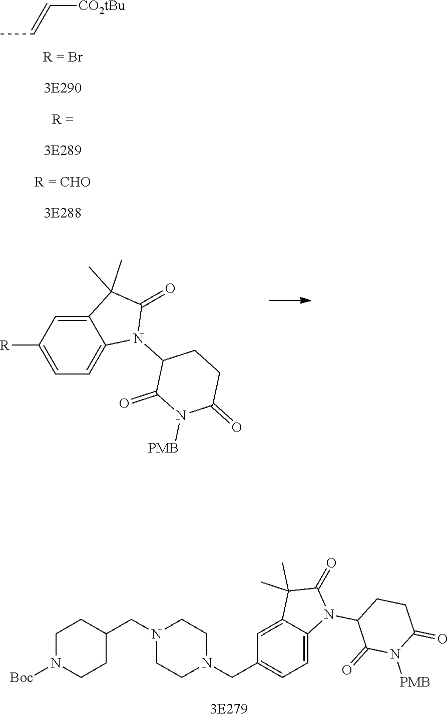

KOtBu (0.68 g) was added to a solution of 5-bromo-3,3-dimethyl-indolin-2-one (1.0 g) in THF (15 mL) at 0° C. Int. 3A8 (2.3 g) was added and stirring continued 2 h at 0° C. The OL (sat. aq. NH₄Cl/EtOAc) was dried, concentrated, and purified by FC (pentane/EtOAc 4:1) to give Int. 3E290 3-(5-bromo-3,3-dimethyl-2-oxo-indolin-1-yl)-1-[(4-methoxyphenyl)methyl]piperidine-2,6-dione (1.0 g). 0.4 g of this material, tert-butyl acrylate (0.33 g), Pd (OAc) 2 (19 mg), and P (o-tol); (52 mg) were degassed in DMF/Et₃N (173:1, 50 mL) and stirred ON at 120° C. The OL (water/EtOAc) was washed with brine, dried, concentrated, and purified by FC (pentane/EtOAc 1:0 to 3:2) to give Int. 3E289 tert-butyl (E)-3-[1-[1-[(4-methoxyphenyl)methyl]-2,6-dioxo-3-piperidyl]-3,3-dimethyl-2-oxo-indolin-5-yl] prop-2-enoate (0.35 g). 0.3 g of this material, NaIO₄ (0.12 g), and OsO₄ (14 mg) were stirred ON in THF/water (2:1; 9 mL). The OL (EtOAc/sat. aq. Na₂S₂O₃) was concentrated and purified by FC (pentane/EtOAc 1:0 to 7:3) to give Int. 3E288 1-[1-[(4-methoxyphenyl)-methyl]-2,6-dioxo-3-piperidyl]-3,3-dimethyl-2-oxo-indoline-5-carbaldehyde (70 mg). Ints. 3E5/3E288 (40 mg/50 mg) were stirred 3 h in DCE/DIPEA (43:1, 3 mL). STAB (46 mg) was added and stirring continued ON at 0° C. to RT. The OL (water/DCM) was concentrated and purified by FC (DCM/MeOH 92:5 to 9:1) to give 3E287 tert-butyl 4-[4-[1-[1-[(4-methoxyphenyl)-methyl]-2,6-dioxo-3-piperidyl]-3,3-dimethyl-2-oxo-indolin-5-yl] methyl]-piperazin-1-yl]methyl]piperidine-1-carboxylate (40 mg).

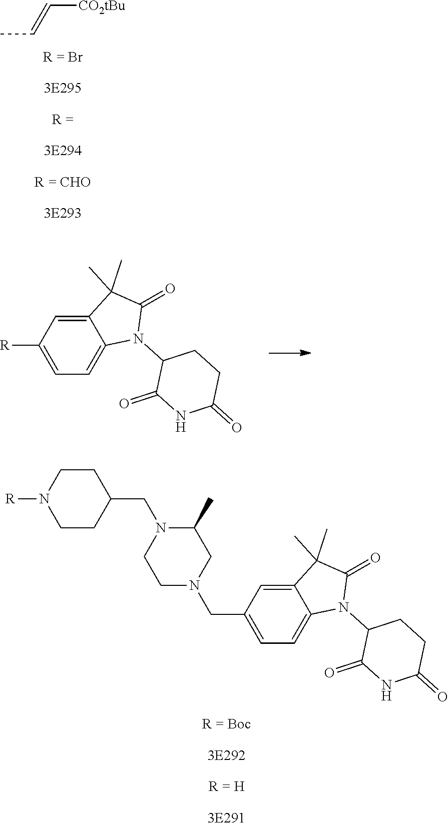

NaH (60% oil dispersion, 1.7 g) was added to a solution of 5-bromo-3,3-dimethyl-indolin-2-one (2.5 g) in THF (10 mL) at 0° C. 3-Bromopiperidine-2,6-dione (8.0 g) was added and stirring continued ON at 60° C. The OL (water/EtOAc) was dried, concentrated, and purified by FC (hexane/EtOAc 7:3 to 1:1) to give Int. 3E295 3-(5-bromo-3,3-dimethyl-2-oxo-indolin-1-yl) piperidine-2,6-dione (1.5 g). Int. 3E295 (1.8 g), tert-butyl acrylate (3.0 mL), Pd (OAc) 2 (92 mg), and P (o-tol)₃ (0.31 g) were degassed in DMF/Et₃N (4.8:1, 12 mL) and stirred ON at 120° C. The OL was (water/EtOAc) washed with brine, dried, and concentrated to give Int. 3E294 tert-butyl (E)-3-[1-(2,6-dioxo-3-piperidyl)-3,3-dimethyl-2-oxo-indolin-5-yl]prop-2-enoate (1.5 g). 0.5 g of this material, NaIO₄ (1.1 g), and OsO₄ (0.1 g) were stirred ON in THF/water (3:1; 20 mL). The OL (EtOAc/sat. aq. Na₂S₂O₃) was washed with sat. aq. NaHCO₃, dried, and concentrated to give Int. 3E293 1-(2,6-dioxo-3-piperidyl)-3,3-dimethyl-2-oxo-indoline-5-carbaldehyde (0.18 g). Ints. 3E74/3E293 (0.25 g/0.10 g) were stirred 5 h in THF/1M ZnCl₂ in THF (1:1, 2 mL). NaCNBH₃ (41 mg) was added and stirring continued ON. The OL (water/EtOAc) was dried, concentrated, and purified by FC (DCM/MeOH 9:1) to give 3E292 tert-butyl 4-[(2S)-4-[1-(2,6-dioxo-3-piperidyl)-3,3-dimethyl-2-oxo-indolin-5-yl]-methyl]-2-methyl-piperazin-1-yl]methyl]piperidine-1-carboxylate (0.11 g). Int. 3E292 (0.12 g) was stirred 3 h in DCM/4M HCl in dioxane (6:1; 6.2 mL) at 0° C. to RT and concentrated to give Int. 3E291 3-(3,3-dimethyl-5-(((S)-3-methyl-4-(piperidin-4-yl-methyl)piperazin-1-yl)methyl)-2-oxoindolin-1-yl) piperidine-2,6-dione (0.11 g, HCl salt).

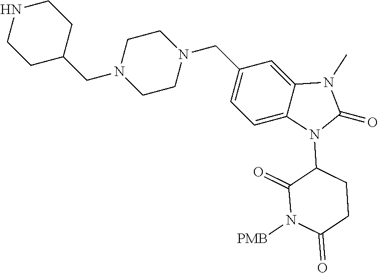

Int. 3E2 (80 mg) was stirred 0.3 h in DCM/TFA (4:1, 2.5 mL) and concentrated to give Int. 3E299 3-[3-methyl-2-oxo-4-[4-(4-piperidylmethyl)piperazin-1-yl]methyl]-benzimidazol-1-yl] piperidine-2,6-dione (85 mg, TFA salt).

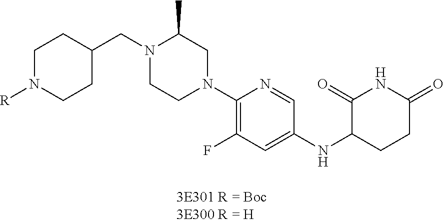

Int. 3E302 (0.65 g) and NaHCO₃ (0.4 g) were stirred in DMF (15 mL) at 0° C. 3-Bromopiperidine-2,6-dione (0.88 g) was added and stirring continued ON at 0° C. to 60° C. The OL (EtOAc/brine and water) was dried, concentrated, and purified by FC (EtOAc) to give Int. 3E301 tert-butyl 4-[[(2R)-4-[5-[(2,6-dioxo-3-piperidyl)-amino]-3-fluoro-2-pyridyl]-2-methyl-piperazin-1-yl] methyl]-piperidine-1-carboxylate (0.6 g). This material was stirred ON in DCM/4M HCl in dioxane (5:4; 9 mL) at 0° C. to RT and concentrated to give Int. 3E300 3-((5-fluoro-6-((S)-3-methyl-4-(piperidin-4-ylmethyl)piperazin-1-yl)pyridin-3-yl)amino)piperidine-2,6-dione (0.45 g, HCl salt).

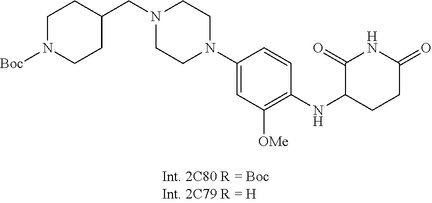 [SMILES: COc1cc(N2CCN(CC3CCN(C(=O)OC(C)(C)C)CC3)CC2)ccc1NC1CCC(=O)NC1=O]

NaHCO₃ (6.2 g), Int. 2C81 (10 g), and 3-bromopiperidine-2,6-dione (10 g) stirred 16 h in ACN (0.5 L) at 90° C. The OL (water/EtOAc) was dried, concentrated, and purified by FC (PE/EtOAc 1:0 to 0:1) to give Int. 2C80 tert-butyl 4-((4-(4-((2,6-dioxopiperidin-3-yl)amino)-3-methoxyphenyl)-piperazin-1-yl)methyl)-piperidine-1-carboxylate (5.5 g). Int. 2C80 (2 g) was stirred 12 h in DCM/4M HCl in dioxane (10:3, 26 mL). The mixture was concentrated and triturated in Et₂O/EtOAc (1:1) to give Int. 2C79 3-((2-methoxy-4-(4-(piperidin-4-ylmethyl)piperazin-1-yl)phenyl)amino)piperidine-2,6-dione (1.3 g, HCl salt).

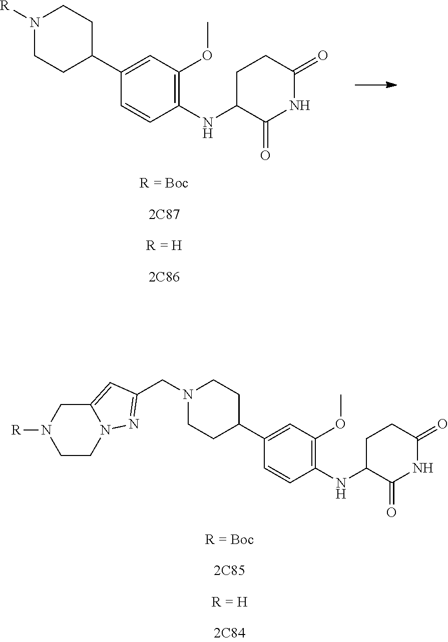

tert-Butyl 4-(4-amino-3-methoxyphenyl) piperidine-1-carboxylate (10 g), 3-bromopiperidine-2,6-dione (12.5 g), and NaHCO₃ (8.2 g) were stirred 18 h in DMF (130 mL) at 80° C. 3-Bromopiperidine-2,6-dione (8.2 g) and NaHCO₃ (5.5 g) were added and stirring continued for 16 h. The mixture was filtered. The OL (water/EtOAc) was dried, concentrated, and purified by FC (PE/EtOAc 1:3) to give Int. 2C87 tert-butyl 4-(4-((2,6-dioxo-piperidin-3-yl)amino)-3-methoxyphenyl) piperidine-1-carboxylate (7 g). Int. 2C87 (7 g) was stirred 5 h in DCM/4M HCl in dioxane (4:3, 140 mL), concentrated, and washed with Et₂O to give Int. 2C86 3-((2-methoxy-4-(piperidin-4-yl)phenyl)amino)piperidine-2,6-dione (5.4 g, HCl salt). Ints. B2Q11/2C86 (2.0 g/4.2 g) were stirred 24 h in DIPEA (8.1 mL) and DMSO (25 mL). STAB (6.7 g) was added at 0° C. and stirring continued for 12 h at 0° C. to RT. The OL (water/EtOAc) was dried, concentrated, and purified by FC (DCM/MeOH 1:0 to 19:1) to give Int. 2C85 tert-butyl 2-((4-(4-((2,6-dioxopiperidin-3-yl)amino)-3-methoxy-phenyl) piperidin-1-yl)methyl)-6,7-dihydropyrazolo[1,5-a]pyrazine-5(4H)-carboxylate (1.7 g). Int. 2C85 (1.8 g) was stirred in DCM/4M HCl in dioxane (1:1, 40 mL) for 5 h at RT, concentrated and washed with Et₂O to give Int. 2C84 3-((2-methoxy-4-(1-((4,5,6,7-tetrahydropyrazolo[1,5-a]pyrazin-2-yl)methyl)piperidin-4-yl)-phenyl)amino)piperidine-2,6-dione (1.3 g, HCl salt).

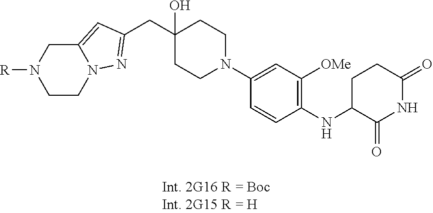

NaHCO₃ (1.1 g), Int. 2G17 (2 g), 3-bromopiperidine-2,6-dione (4.2 g) were stirred 24 h in DMF (3 mL) at 70° C. The OL (water/DCM/MeOH) was dried, filtered, concentrated, and purified by FC (DCM/MeOH 19:1) to give Int. 2G16 tert-butyl 2-((1-(4-((2,6-dioxopiperidin-3-yl)amino)-3-methoxyphenyl)-4-hydroxypiperidin-4-yl)methyl)-6,7-dihydropyrazolo-[1,5-a]pyrazine-5(4H)-carboxylate (1 g). Int. 2G16 (0.90 g) was stirred 5 h in DCM/4M HCl in dioxane (2:1, 12 mL) at 0° C. to RT. The residue after concentration was washed with Et₂O to give Int. 2G15 3-((4-(4-hydroxy-4-((4,5,6,7-tetrahydropyrazolo[1,5-a]pyrazin-2-yl)methyl)piperidin-1-yl)-2-methoxyphenyl)amino)piperidine-2,6-dione (0.80 g, HCl salt).

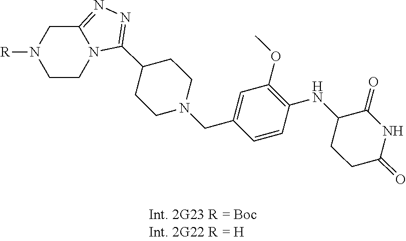

Int. 2G24 (0.15 g), 3-bromopiperidine-2,6-dione (0.25 g) and NaHCO₃ (0.22 g) were stirred ON in DMF (3 mL) at 90° C. The mixture was concentrated and HPLC-purified to give Int. 2G23 tert-butyl 3-(1-(4-((2,6-dioxo-piperidin-3-yl)amino)-3-methoxybenzyl) piperidin-4-yl)-5,6-dihydro-[1,2,4]triazolo[4,3-a]pyrazine-7 (8H)-carboxylate (71 mg). Int. 2G23 (71 mg) was stirred in DCM/TFA (2:1, 1.5 mL) ON at RT and concentrated to give Int. 2G22 3-((2-methoxy-4-((4-(5,6,7,8-tetrahydro-[1,2,4]triazolo-[4,3-a]pyrazin-3-yl) piperidin-1-yl)methyl) phenyl)amino)piperidine-2,6-dione (80 mg, TFA salt).

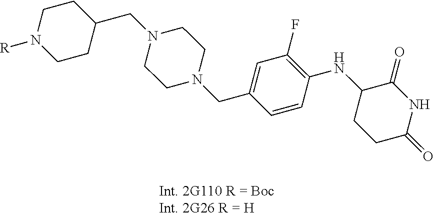

NaHCO₃ (3.0 g), Int. 2G111 (1.5 g), and 3-bromopiperidine-2,6-dione (2.0 g) were stirred 48 h in DMF (40 mL) at 80° C. The mixture was filtered and concentrated to give Int. 2G110 tert-butyl 4-((4-(4-((2,6-dioxopiperidin-3-yl)amino)-3-fluorobenzyl) piperazin-1-yl)methyl)-piperidine-1-carboxylate (1.7 g). Int. 2G110 (1.7 g) was stirred ON in DCM/TFA (6:1, 35 mL). The mixture was concentrated and HPLC-purified to give Int. 2G26 3-((2-fluoro-4-((4-(piperidin-4-ylmethyl)piperazin-1-yl)methyl) phenyl)amino)piperidine-2,6-dione (0.12 g, TFA salt).

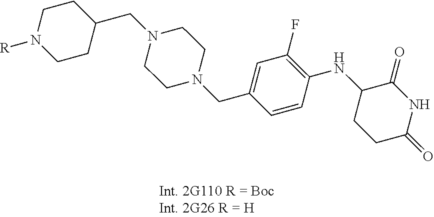

NaHCO₃ (3.0 g), Int. 2G111 (1.5 g), and 3-bromopiperidine-2,6-dione (2.0 g) were stirred 48 h in DMF (40 mL) at 80° C. The mixture was filtered and concentrated to give Int. 2G110 tert-butyl 4-((4-(4-((2,6-dioxopiperidin-3-yl)amino)-3-fluorobenzyl) piperazin-1-yl)methyl)-piperidine-1-carboxylate (1.7 g). Int. 2G110 (1.7 g) was stirred ON in DCM/TFA (6:1, 35 mL). The mixture was concentrated and HPLC-purified to give Int. 2G26 3-((2-fluoro-4-((4-(piperidin-4-ylmethyl)piperazin-1-yl)methyl) phenyl)amino)piperidine-2,6-dione (0.12 g, TFA salt).

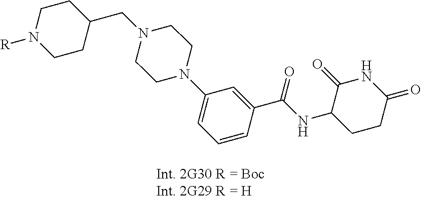

Int. 2G31 (3.2 g) and 3-aminopiperidine-2,6-dione (1.6 g) were mixed in DMF/Et₃N (1:1, 21 mL). T3P (50% in EtOAc, 23 mL) was added and the mixture was stirred 16 h at 0° C. to RT. The OL (water/EtOAc) was dried, filtered, concentrated and purified by FC (hexane/EtOAc 2:3) to give Int. 2G30 tert-butyl 4-((4-(3-((2,6-dioxopiperidin-3-yl) carbamoyl)phenyl)-piperazin-1-yl)methyl)piperidine-1-carboxylate (2.3 g). Int. 2G30 (2.2 g) was stirred 16 h in DCM/4M HCl in dioxane (5:3, 48 mL) at 0° C. to RT. The mixture was concentrated and washed with Et₂O to give Int. 2G29 N-(2,6-dioxopiperidin-3-yl)-3-(4-(piperidin-4-ylmethyl)piperazin-1-yl)benzamide (1.8 g, HCl salt).

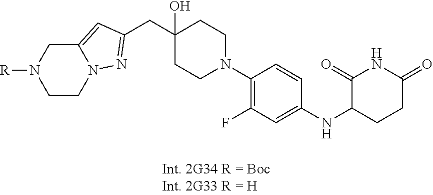

NaHCO₃ (0.12 g), Int. 2G35 (65 mg), and 3-bromopiperidine-2,6-dione (0.14 g) were stirred ON in DMF (3 mL) at 90° C. The mixture was concentrated and HPLC-purified to give Int. 2G34 tert-butyl 2-((1-(4-((2,6-dioxopiperidin-3-yl)amino)-2-fluorophenyl)-4-hydroxypiperidin-4-yl)methyl)-6,7-dihydropyrazolo[1,5-a]pyrazine-5(4H)-carboxylate (24 mg). Int. 2G34 (24 mg) was stirred 2 h in DCM/TFA (4:1, 2.5 mL) and concentrated to give Int. 2G33 3-((3-fluoro-4-(4-hydroxy-4-((4,5,6,7-tetrahydropyrazolo[1,5-a]pyrazin-2-yl)methyl)piperidin-1-yl)phenyl)amino)piperidine-2,6-dione (25 mg, TFA salt).

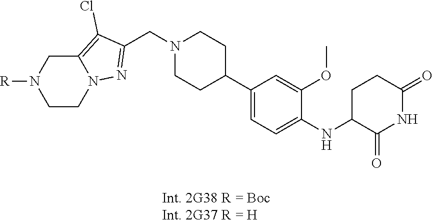

Ints. 2G39/2C86 (0.67 g/0.75 g) and 4 Å MS were stirred ON in DCE/Et₃N (17:1, 10.6 mL). NaCNBH₃ (0.17 g) was added and stirring continued for 3 h. The mixture was filtered, concentrated, and HPLC-purified to give Int. 2G38 tert-butyl 3-chloro-2-((4-(4-((2,6-dioxopiperidin-3-yl)amino)-3-methoxy-phenyl) piperidin-1-yl)-methyl)-6,7-dihydropyrazolo[1,5-a]pyrazine-5(4H)-carboxylate (90 mg). Int. 2G38 (90 mg) was stirred 20 h in DCM/TFA (34:1, 2.06 mL) and concentrated to give Int. 2G37 3-((4-(1-((3-chloro-4,5,6,7-tetrahydropyrazolo-[1,5-a]pyrazin-2-yl)methyl)piperidin-4-yl)-2-methoxyphenyl)amino)piperidine-2,6-dione (90 mg, TFA salt).

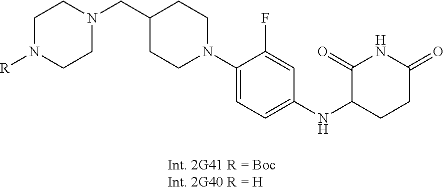

Int. 2G42 (1.5 g), 3-bromopiperidine-2,6-dione (3.7 g), and NaHCO₃ (1.6 g) were stirred ON in DMF (50 mL) at 80° C. The mixture was concentrated. The OL (water/EtOAc) was concentrated and HPLC-purified to give Int. 2G41 tert-butyl 4-((1-(4-((2,6-dioxopiperidin-3-yl)amino)-2-fluorophenyl) piperidin-4-yl)methyl)-piperazine-1-carboxylate (40 mg). Int. 2G41 (40 mg) was stirred 3 h in MeOH/4M HCl in dioxane (5:1, 12 mL) and concentrated to give Int. 2G40 3-((3-fluoro-4-(4-(piperazin-1-ylmethyl)piperidin-1-yl)phenyl)amino)piperidine-2,6-dione (32 mg, HCl salt).

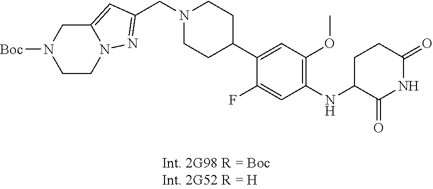 [SMILES: COc1cc(C2CCN(Cc3cc4n(n3)CCN(C(=O)OC(C)(C)C)C4)CC2)c(F)cc1NC1CCC(=O)NC1=O]

Ints. 2Q11/2G100 (0.1 g/0.1 g) and STAB (0.3 g) were stirred 48 h in DCE/DIPEA (150:1, 15.1 mL). The OL (water/DCE) was dried, filtered, and concentrated to give Int. 2G98 tert-butyl 2-((4-(4-((2,6-dioxopiperidin-3-yl)amino)-2-fluoro-5-methoxyphenyl) piperidin-1-yl)methyl)-6,7-dihydropyrazolo[1,5-a]pyrazine-5(4H)-carboxylate (0.2 g). Int. 2G98 (0.2 g) was stirred ON in DCM/TFA (5:1, 18 mL), concentrated, and HPLC-purified to give Int. 2G52 3-((5-fluoro-2-methoxy-4-(1-((4,5,6,7-tetrahydropyrazolo[1,5-a]pyrazin-2-yl)methyl)piperidin-4-yl)phenyl)amino)piperidine-2,6-dione (50 mg).

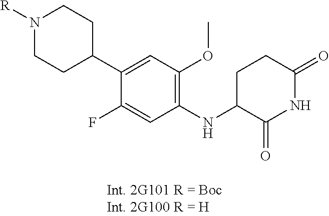

Int. 2G102 (1.5 g), 3-bromopiperidine-2,6-dione (2.2 g), and NaHCO₃ (2 g) were stirred 48 h in DMF (50 mL) at 80° C. The mixture was filtered, concentrated, and purified by FC (hexane/THF 1:0 to 0:1) to give Int. 2G101 tert-butyl 4-(4-((2,6-dioxopiperidin-3-yl)amino)-2-fluoro-5-methoxyphenyl) piperidine-1-carboxylate (0.15 g). Int. 2G101 (0.15 g) was stirred ON in DCM/TFA (10:1, 11 mL) and concentrated. The OL (aq. NaHCO)/EtOAc) was concentrated to give Int. 2G100 3-((5-fluoro-2-methoxy-4-(piperidin-4-yl)phenyl)amino)piperidine-2,6-dione (0.1 g).

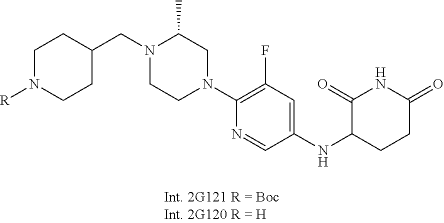

Int. 2G122 (10 g), 3-bromopiperidine-2,6-dione (31 g), and NaHCO₃ (7.3 g) were stirred 72 h in ACN (180 mL) at 80° C. The OL (water/EtOAc) was dried, concentrated, and purified by FC (PE/EtOAc 1:4 to 6:7) to give Int. 2G121 tert-butyl 4-(((2R)-4-(5-((2,6-dioxopiperidin-3-yl)amino)-3-fluoropyridin-2-yl)-2-methylpiperazin-1-yl)methyl)piperidine-1-carboxylate (6 g). Int. 2G121 (1.0 g) was stirred 6 h in 2M HCl in dioxane (20 mL) at 0° C. to RT. The mixture was concentrated and washed with Et₂O to give Int. 2G120 3-((5-fluoro-6-((R)-3-methyl-4-(piperidin-4-ylmethyl)piperazin-1-yl)pyridin-3-yl)amino)piperidine-2,6-dione (0.75 g, HCl salt).

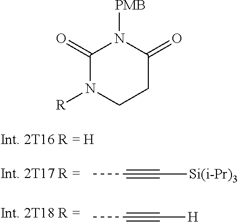

1-(Chloromethyl)-4-methoxybenzene (31 mL) was added dropwise to a solution of dihydropyrimidine-2,4 (1H,3H)-dione (20 g) and Cs₂CO₃ (68 g) in DMF (40 mL) at 0° C. and stirred for 16 h. The reaction mixture was diluted with water to precipitate a solid that was purified by FC (PE/EtOAc 1:1 to 2:3) to give Int. 2T16 3-(4-methoxybenzyl) dihydropyrimidine-2,4 (1H,3H)-dione (16 g). Int. 2T16 (0.50 g), (bromoethynyl) triisopropylsilane (1.1 g), CuSO₄ (34 mg), 1,10-phenanthroline (77 mg) and K₃PO₄ (1.4 g) were stirred ON in toluene (4 mL) at 80° C. The OL (water/EtOAc) was concentrated and purified by FC (heptane/EtOAc 1:0 to 1:1) to give Int. 2T17 3-(4-methoxybenzyl)-1-((triisopropylsilyl) ethynyl)-dihydropyrimidine-2,4 (1H,3H)-dione (0.69 g). IM TBAF in THF (3.1 mL) was added to a solution of Int. 2T17 (0.69 g) in THF (8 mL) at −10° C. and stirred for 30 min at RT. The OL (water/EtOAc) was concentrated and purified by FC (heptane/EtOAc 4:1 to 2:3) to give Int. 2T18 1-ethynyl-3-(4-methoxybenzyl) dihydropyrimidine-2,4 (1H,3H)-dione (0.26 g).

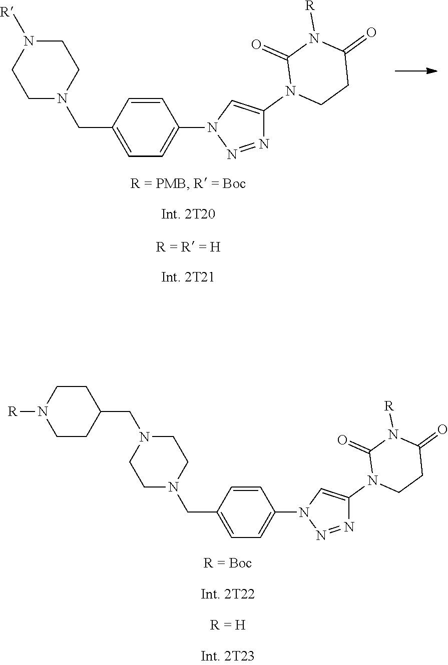

A mixture of CuSO₄ (92 mg) and sodium ascorbate (0.23 g) in H₂O (3 mL) was added to Ints. 2T19/2T18 (0.23 g/0.21 g) in THF (4 mL) and stirred for 48 h. The mixture was concentrated and purified by FC (heptane/EtOAc 1:0 to 0:1) to give Int. 2T20 tert-butyl 4-(4-(4-(3-(4-methoxybenzyl)-2,4-dioxotetra-hydropyrimidin-1(2H)-yl)-1H-1,2,3-triazol-1-yl)benzyl) piperazine-1-carboxylate (0.31 g). Int. 2T20 (0.31 g) and TfOH (0.57 mL) were stirred in TFA (5 mL) for 40 min at 80° C. and concentrated. The residue was dissolved in ACN/sat. aq. NaHCO₃, concentrated and purified by FC (DCM/MeOH 1:0 to 3:1) to give Int. 2T21 1-(1-(4-(piperazin-1-ylmethyl)phenyl)-1H-1,2,3-triazol-4-yl) dihydropyrimidine-2,4 (1H,3H)-dione (0.50 g). Int. 2T21 (0.50 g) and tert-butyl 4-formylpiperidine-1-carboxylate (0.60 g) were stirred in DMF/DIPEA (13:2, 3.5 mL) for 15 min under an inert atmosphere. STAB (0.45 g) was added and stirring continued ON. The OL (water/EtOAc) was dried, filtered, concentrated and purified by FC (EtOAc/5% Et₃N in MeOH 1:0 to 7:1) to give Int. 2T22 tert-butyl 4-((4-(4-(4-(2,4-dioxotetrahydropyrimidin-1(2H)-yl)-1H-1,2,3-triazol-1-yl)benzyl) piperazin-1-yl)methyl)piperidine-1-carboxylate (0.17 g). Int. 2T22 (0.17 g) was stirred in DCM/TFA (1:1, 6 mL) for 30 min and concentrated to give Int. 2T23 1-(1-(4-((4-(piperidin-4-ylmethyl)piperazin-1-yl)methyl)phenyl)-1H-1,2,3-triazol-4-yl) dihydropyrimidine-2,4 (1H,3H)-dione (0.21 g, TFA salt).

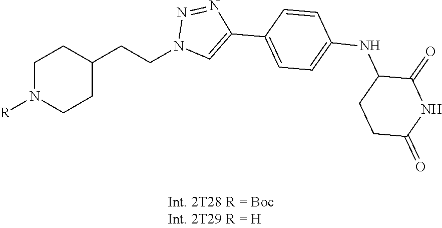

Int. 2T27 (2.0 g), 3-bromopiperidine-2,6-dione (2.4 g) and 1M NaHCO; (22 mL) were stirred in ACN (25 mL) for 12 h at 100° C. and concentrated. The OL (water/EtOAc) was concentrated, combined with two smaller batches prepared similarly and purified by FC (hexane/EtOAc) to give Int. 2T28 tert-butyl 4-(2-(4-(4-((2,6-dioxopiperidin-3-yl)amino)phenyl)-1H-1,2,3-triazol-1-yl)ethyl)piperidine-1-carboxylate (0.45 g). Int. 2T28 (0.45 g) was mixed in DCM/4M HCl in dioxane (2:1, 15 mL) at 0° C. and stirred for 6 h at RT. The mixture was concentrated and washed with Et₂O to give Int. 2T29 3-((4-(1-(2-(piperidin-4-yl)ethyl)-1H-1,2,3-triazol-4-yl)phenyl)amino)piperidine-2,6-dione (0.38 g, HCl salt).

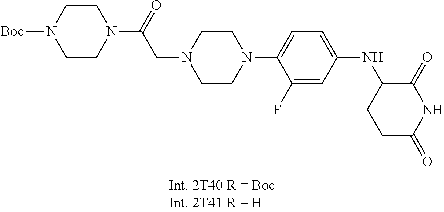 [SMILES: CC(C)(C)OC(=O)N1CCN(C(=O)CN2CCN(c3ccc(NC4CCC(=O)NC4=O)cc3F)CC2)CC1]

Int. 2T40 tert-butyl 4-(2-(4-(4-((2,6-dioxopiperidin-3-yl)amino)-2-fluorophenyl) piperazin-1-yl) acetyl)-piperazine-1-carboxylate (0.70 g) was prepared similarly to Int. 2G30 from tert-butyl piperazine-1-carboxylate (0.84 g) and Int. 2A6 (1.1 g). Int. 2T409 (0.70 g) was mixed in DCM/4M HCl in dioxane (7:4, 11 mL) at 0° C. and stirred for 12 h at RT. The mixture was concentrated and washed with Et₂O to give Int. 2T41 3-((3-fluoro-4-(4-(2-oxo-2-(piperazin-1-yl)ethyl) piperazin-1-yl)phenyl)amino)piperidine-2,6-dione (0.60 g, HCl salt).

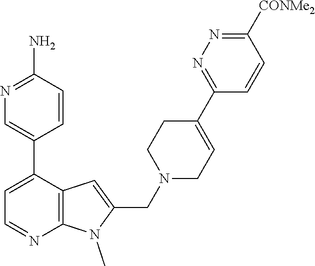

### Example 1z3. 6-(1-((4-(6-aminopyridin-3-yl)-1-methyl-1H-pyrrolo[2,3-b]pyridin-2-yl)methyl)-1,2,3,6-tetrahydropyridin-4-yl)-N,N-dimethylpyridazine-3-carboxamide

PdDPPFCl₂-DCM (10 mg), Int. 1Z14 (70 mg), (6-aminopyridin-3-yl) boronic acid (50 mg), and K₂CO₃ (70 mg) were stirred 12 h in dioxane/H2O (5:1, 6 mL) at 80° C. under an inert atmosphere. The OL (water/EtOAc) was concentrated and HPLC-purified to give Example 1z3 (30 mg).

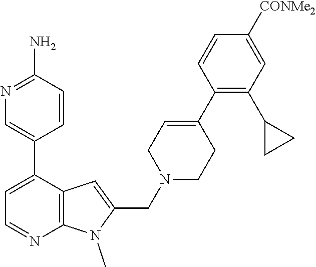

### Example 1z23. 4-(1-((4-(6-aminopyridin-3-yl)-1-methyl-1H-pyrrolo[2,3-b]pyridin-2-yl)methyl)-1,2,3,6-tetrahydropyridin-4-yl)-3-cyclopropyl-N,N-dimethylbenzamide

Example 1z23 (0.13 g) was prepared similarly to Example 1z3 from Int. 1Z109 (0.20 g) and S-(4,4,5,5-tetramethyl-1,3,2-dioxaborolan-2-yl)pyridin-2-amine (0.10 g).

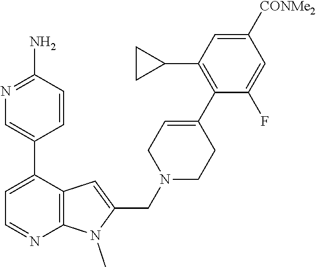

### Example 1z35. 4-(1-((4-(6-aminopyridin-3-yl)-1-methyl-1H-pyrrolo[2,3-b]pyridin-2-yl)methyl)-1,2,3,6-tetrahydropyridin-4-yl)-3-cyclopropyl-5-fluoro-N,N-dimethylbenzamide

Example 1z35 (22 mg) was prepared similarly to Example 1z3 from Int. 1Z124 (0.20 g) and 5-(4,4,5,5-tetramethyl-1,3,2-dioxaborolan-2-yl)pyridin-2-amine (0.10 g).

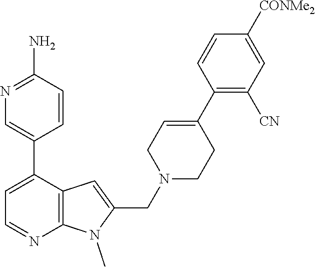

### Example 1z10. 4-(1-((4-(6-aminopyridin-3-yl)-1-methyl-1H-pyrrolo[2,3-b]pyridin-2-yl)methyl)-1,2,3,6-tetrahydropyridin-4-yl)-3-cyano-N,N-dimethylbenzamide

Int. 1Z23 (60 mg) was stirred ON in 2.8M HCl in dioxane (10 mL). The mixture was concentrated and HPLC-purified to give Example 1z10 (9 mg).

### Example 2. Preparation of Compounds

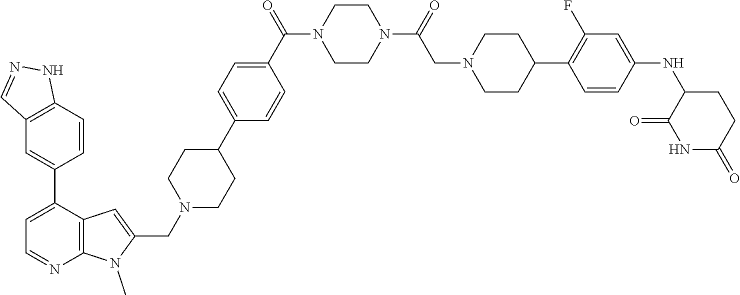 [SMILES: Cn1c(CN2CCC(c3ccc(C(=O)N4CCN(C(=O)CN5CCC(c6ccc(NC7CCC(=O)NC7=O)cc6F)CC5)CC4)cc3)CC2)cc2c(-c3ccc4[nH]ncc4c3)ccnc21]

### Example 2a1. 3-((4-(1-(2-(4-(4-(1-((4-(1H-indazol-5-yl)-1-methyl-1H-pyrrolo[2,3-b]pyridin-2-yl)methyl)-piperidin-4-yl)benzoyl) piperazin-1-yl)-2-oxoethyl)piperidin-4-yl)-3-fluorophenyl)amino)piperidine-2,6-dione

Ints. 2A1/2A3 (27 mg/28 mg, TFA salt), and HATU (23 mg) were stirred ON in DMSO/DIPEA (11:1, 1.1 mL). The mixture was HPLC-purified to give Example 2a1 (20 mg).

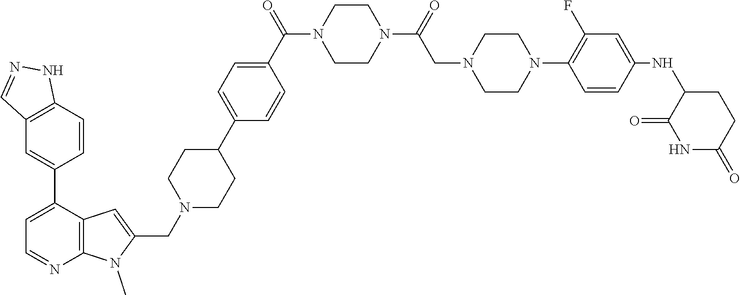 [SMILES: Cn1c(CN2CCC(c3ccc(C(=O)N4CCN(C(=O)CN5CCN(c6ccc(NC7CCC(=O)NC7=O)cc6F)CC5)CC4)cc3)CC2)cc2c(-c3ccc4[nH]ncc4c3)ccnc21]

### Example 2a2. 3-((4-(4-(2-(4-(4-(1-((4-(1H-indazol-5-yl)-1-methyl-1H-pyrrolo[2,3-b]pyridin-2-yl)methyl)-piperidin-4-yl)benzoyl) piperazin-1-yl)-2-oxoethyl) piperazin-1-yl)-3-fluorophenyl)amino)piperidine-2,6-dione

Example 2a2 (22 mg) prepared similarly to Example 2b1 from Ints. 2A1/2A6 (27 mg/40 mg, TFA salt).

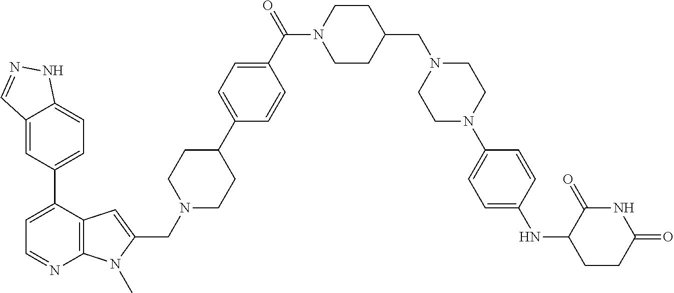 [SMILES: Cn1c(CN2CCC(c3ccc(C(=O)N4CCC(CN5CCN(c6ccc(NC7CCC(=O)NC7=O)cc6)CC5)CC4)cc3)CC2)cc2c(-c3ccc4[nH]ncc4c3)ccnc21]

### Example 2b1. 3-((4-(4-((1-(4-(1-((4-(1H-indazol-5-yl)-1-methyl-1H-pyrrolo[2,3-b]pyridin-2-yl)methyl)piperidin-4-yl)benzoyl)piperidin-4-yl)methyl)piperazin-1-yl)phenyl)amino)piperidine-2,6-dione

Example 2b1 (16 mg) prepared similarly to Example 2a1 from HCl salts of Ints. 3A1/2B1 (27 mg/19 mg) using DMF instead of DMSO.

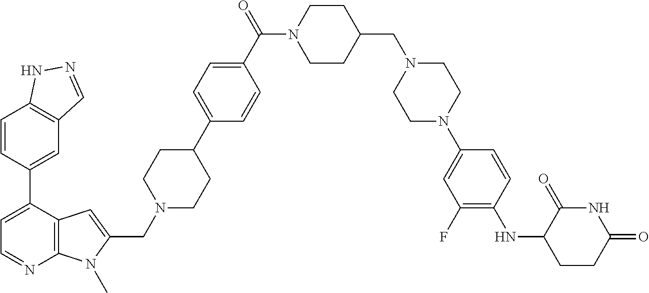 [SMILES: Cn1c(CN2CCC(c3ccc(C(=O)N4CCC(CN5CCN(c6ccc(NC7CCC(=O)NC7=O)c(F)c6)CC5)CC4)cc3)CC2)cc2c(-c3ccc4[nH]ncc4c3)ccnc21]

### Example 2b2. 3-((4-(4-((1-(4-(1-((4-(1H-indazol-5-yl)-1-methyl-1H-pyrrolo[2,3-b]pyridin-2-yl)methyl)piperidin-4-yl)benzoyl)piperidin-4-yl)methyl)piperazin-1-yl)-2-fluorophenyl)amino)piperidine-2,6-dione

Example 2b2 (7 mg) prepared similarly to Example 2b1 from Ints. 3A1/2B3 (23 mg, TFA salt/10 mg, HCl salt).

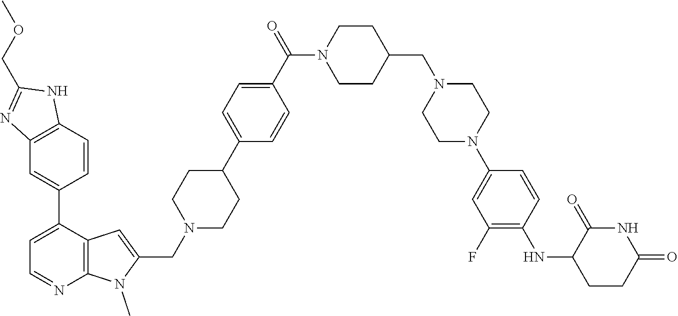 [SMILES: COCc1nc2cc(-c3ccnc4c3cc(CN3CCC(c5ccc(C(=O)N6CCC(CN7CCN(c8ccc(NC9CCC(=O)NC9=O)c(F)c8)CC7)CC6)cc5)CC3)n4C)ccc2[nH]1]

### Example 2b3. 3-((2-fluoro-4-(4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperazin-1-yl)phenyl)amino)piperidine-2,6-dione

Example 2b3 (5 mg) prepared similarly to Example 2b1 from HCl salts of Ints. 3E261/2B3 (7 mg/5 mg).

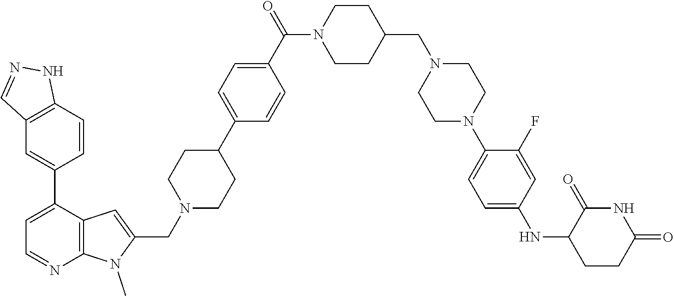 [SMILES: Cn1c(CN2CCC(c3ccc(C(=O)N4CCC(CN5CCN(c6ccc(NC7CCC(=O)NC7=O)cc6F)CC5)CC4)cc3)CC2)cc2c(-c3ccc4[nH]ncc4c3)ccnc21]

### Example 2c1. 3-((4-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)-3-fluorophenyl)amino)piperidine-2,6-dione

Example 2c1 (21 mg) prepared similarly to Example 2b1 from TFA salts of Ints. 3A1/2C15 (25 mg/31 mg).

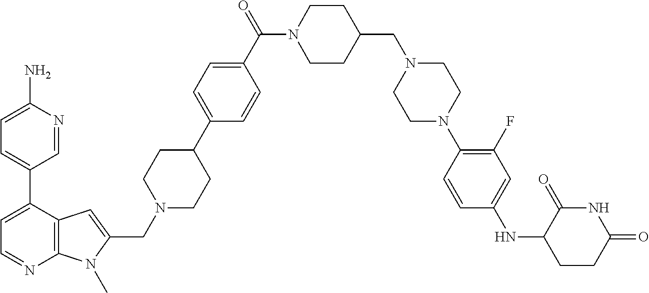 [SMILES: Cn1c(CN2CCC(c3ccc(C(=O)N4CCC(CN5CCN(c6ccc(NC7CCC(=O)NC7=O)cc6F)CC5)CC4)cc3)CC2)cc2c(-c3ccc(N)nc3)ccnc21]

### Example 2c2. 3-((4-(4-((1-(4-(1-((4-(6-aminopyridin-3-yl)-1-methyl-1H-pyrrolo[2,3-b]pyridin-2-yl)methyl)-piperidin-4-yl)benzoyl)piperidin-4-yl)methyl)piperazin-1-yl)-3-fluorophenyl)amino)piperidine-2,6-dione

Example 2c2 (22 mg) prepared similarly to Example 2b1 from Ints. 3E23/2C15 (30 mg/43 mg, TFA salt).

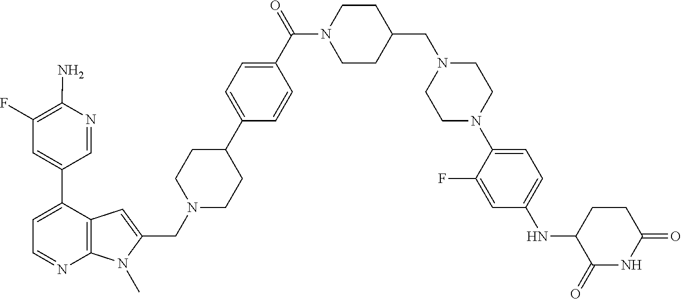 [SMILES: Cn1c(CN2CCC(c3ccc(C(=O)N4CCC(CN5CCN(c6ccc(NC7CCC(=O)NC7=O)cc6F)CC5)CC4)cc3)CC2)cc2c(-c3cnc(N)c(F)c3)ccnc21]

### Example 2c3. 3-((4-(4-((1-(4-(1-((4-(6-amino-5-fluoropyridin-3-yl)-1-methyl-1H-pyrrolo[2,3-b] pyridin-2-yl)-methyl)piperidin-4-yl)benzoyl)piperidin-4-yl)methyl)piperazin-1-yl)-3-fluorophenyl)amino)piperidine-2,6-dione

Example 2c3 (19 mg) prepared similarly to Example 2b1 from Ints. 2C1/2C15 (30 mg/46 mg, TFA salt).

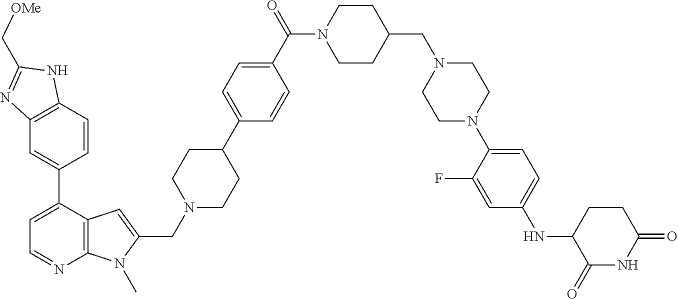 [SMILES: COCc1nc2cc(-c3ccnc4c3cc(CN3CCC(c5ccc(C(=O)N6CCC(CN7CCN(c8ccc(NC9CCC(=O)NC9=O)cc8F)CC7)CC6)cc5)CC3)n4C)ccc2[nH]1]

### Example 2c4. 3-((3-fluoro-4-(4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperazin-1-yl)phenyl)-amino)piperidine-2,6-dione

Example 2c4 (5 mg) prepared similarly to Example 2b1 from Ints. 3E261/2C15 (14 mg/18 mg, TFA salt).

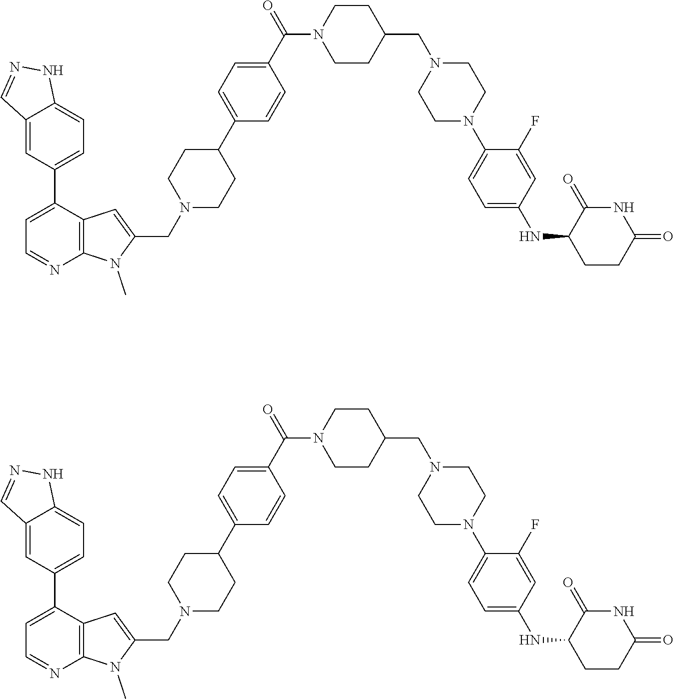 [SMILES: Cn1c(CN2CCC(c3ccc(C(=O)N4CCC(CN5CCN(c6ccc(N[C@@H]7CCC(=O)NC7=O)cc6F)CC5)CC4)cc3)CC2)cc2c(-c3ccc4[nH]ncc4c3)ccnc21 ; Cn1c(CN2CCC(c3ccc(C(=O)N4CCC(CN5CCN(c6ccc(N[C@H]7CCC(=O)NC7=O)cc6F)CC5)CC4)cc3)CC2)cc2c(-c3ccc4[nH]ncc4c3)ccnc21]

### Examples 2c5 and 2c6. (R)-3-((4-(4-((1-(4-(1-((4-(1H-indazol-5-yl)-1-methyl-1H-pyrrolo[2,3-b] pyridin-2-yl)methyl)-piperidin-4-yl)benzoyl)piperidin-4-yl)methyl)piperazin-1-yl)-3-fluorophenyl)amino)piperidine-2,6-dione and(S)-3-((4-(4-((1-(4-(1-((4-(1H-indazol-5-yl)-1-methyl-1H-pyrrolo[2,3-b]pyridin-2-yl)methyl)piperidin-4-yl)benzoyl)piperidin-4-yl)-methyl)piperazin-1-yl)-3-fluorophenyl)amino)piperidine-2,6-dione

Example 2c1 (0.25 g) was resolved by HPLC using a Chiralpak IA 250×30 5 μm column operated at RT and an eluent of 70% 0.2% aq. TFA and 30% ACN (1 mL/min) to give Examples 2c5 (30 mg, first peak) and 2c6 (18 mg, second peak). The absolute configurations of these compounds at the glutarimide chiral centers were not determined.

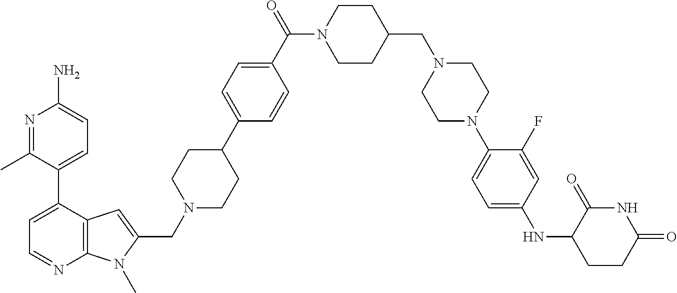 [SMILES: Cc1nc(N)ccc1-c1ccnc2c1cc(CN1CCC(c3ccc(C(=O)N4CCC(CN5CCN(c6ccc(NC7CCC(=O)NC7=O)cc6F)CC5)CC4)cc3)CC1)n2C]

### Example 2c7. 3-((4-(4-((1-(4-(1-((4-(6-amino-2-methylpyridin-3-yl)-1-methyl-1H-pyrrolo[2,3-b] pyridin-2-yl)methyl)piperidin-4-yl)benzoyl)piperidin-4-yl)methyl)piperazin-1-yl)-3-fluorophenyl)amino)piperidine-2,6-dione

Int. 2C38 (17 mg), 5-bromo-6-methyl-pyridin-2-amine (5 mg), PdDPPFCl₂-DCM (2 mg), and NaHCO₃ (6 mg) were degassed in DMF/DMSO/water (5:1:1, 1.4 mL) and stirred 5 h at 90° C. The mixture was HPLC-purified to give Example 2c7 (5.7 mM in DMSO (0.2 mL)).

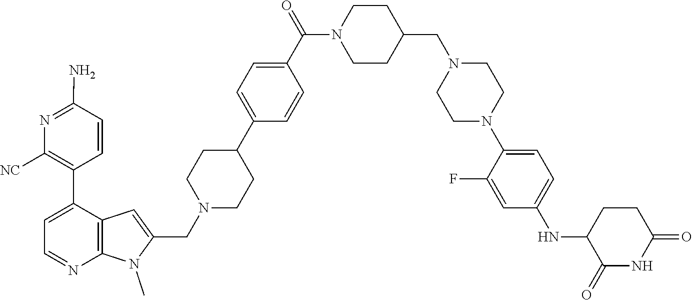 [SMILES: Cn1c(CN2CCC(c3ccc(C(=O)N4CCC(CN5CCN(c6ccc(NC7CCC(=O)NC7=O)cc6F)CC5)CC4)cc3)CC2)cc2c(-c3ccc(N)nc3C#N)ccnc21]

### Example 2c8. 6-amino-3-(2-((4-(4-(4-((4-(4-((2,6-dioxopiperidin-3-yl)amino)-2-fluorophenyl) piperazin-1-yl)-methyl)piperidine-1-carbonyl)phenyl) piperidin-1-yl)methyl)-1-methyl-1H-pyrrolo[2,3-b]pyridin-4-yl)-picolinonitrile

Int. 2C38 (18 mg), NaHCO₃ (6 mg), 6-amino-3-bromopicolinonitrile (171 mg), and PdDPPFCl₂-DCM (2 mg) were degassed in dioxane/water/DMF (1:1:2.5, 1.4 mL) before stirring 5 h at 90° C. The residue after concentration was HPLC-purified to give Example 2c8 (10 mM in DMSO (0.3 mL)).

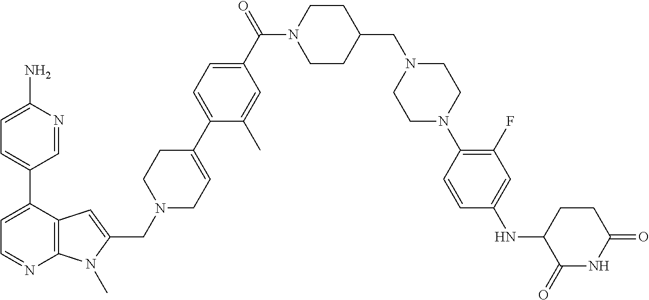 [SMILES: Cc1cc(C(=O)N2CCC(CN3CCN(c4ccc(NC5CCC(=O)NC5=O)cc4F)CC3)CC2)ccc1C1=CCN(Cc2cc3c(-c4ccc(N)nc4)ccnc3n2C)CC1]

### Example 2c9. 3-((4-(4-((1-(4-(1-((4-(6-aminopyridin-3-yl)-1-methyl-1H-pyrrolo[2,3-b]pyridin-2-yl)methyl)-1,2,3,6-tetrahydropyridin-4-yl)-3-methylbenzoyl)piperidin-4-yl)methyl)piperazin-1-yl)-3-fluorophenyl)-amino)piperidine-2,6-dione

Example 2c9 (15 mg) prepared similarly to Example 2c15 from Int. 2C45 (27 mg).

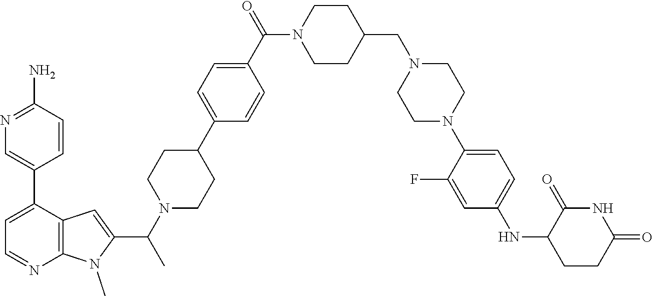 [SMILES: CC(c1cc2c(-c3ccc(N)nc3)ccnc2n1C)N1CCC(c2ccc(C(=O)N3CCC(CN4CCN(c5ccc(NC6CCC(=O)NC6=O)cc5F)CC4)CC3)cc2)CC1]

### Example 2c10. 3-((4-(4-((1-(4-(1-(1-(4-(6-aminopyridin-3-yl)-1-methyl-1H-pyrrolo[2,3-b] pyridin-2-yl)-ethyl)-piperidin-4-yl)benzoyl)piperidin-4-yl)methyl)piperazin-1-yl)-3-fluorophenyl)amino)piperidine-2,6-dione

5-(4,4,5,5-tetramethyl-1,3,2-dioxaborolan-2-yl)pyridin-2-amine (91 mg), PdDPPFCl₂-DCM (17 mg), NaHCO₃ (87 mg), and Int. 2C53 (171 mg) were degassed in dioxane/water (6.7:1, 2.3 mL) and stirred ON at 90° C. The OL (DCM/MeOH (9:1)/water) was dried, concentrated, and purified by FC (DCM/MeOH/Et₃N 1:0:0 to 40:10:1) and by HPLC to give Example 2c10 (10 mM in DMSO (1.09 mL)).

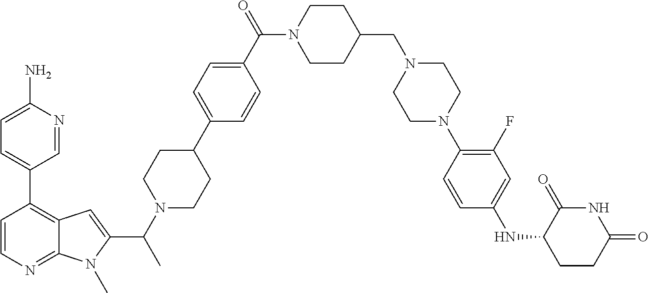 [SMILES: CC(c1cc2c(-c3ccc(N)nc3)ccnc2n1C)N1CCC(c2ccc(C(=O)N3CCC(CN4CCN(c5ccc(N[C@H]6CCC(=O)NC6=O)cc5F)CC4)CC3)cc2)CC1]

### Example 2c11. (3R)-3-((4-(4-((1-(4-(1-(1-(4-(6-aminopyridin-3-yl)-1-methyl-1H-pyrrolo[2,3-b] pyridin-2-yl)ethyl)piperidin-4-yl)benzoyl)piperidin-4-yl)methyl)piperazin-1-yl)-3-fluorophenyl)amino)piperidine-2,6-dione

Example 2c11 (56 mg) prepared similarly to Example 2b1 from Ints. 2C9/2C12 (48 mg/55 mg, HCl salt). The absolute configuration at the glutarimide stereocenter was not determined.

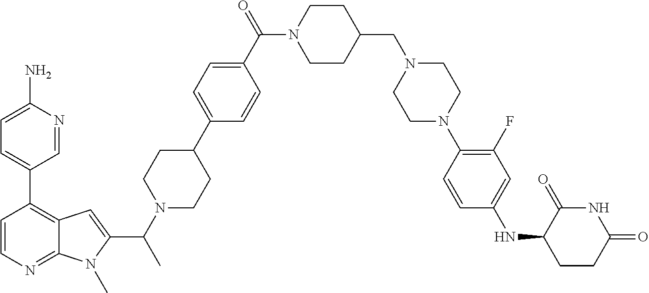 [SMILES: CC(c1cc2c(-c3ccc(N)nc3)ccnc2n1C)N1CCC(c2ccc(C(=O)N3CCC(CN4CCN(c5ccc(N[C@@H]6CCC(=O)NC6=O)cc5F)CC4)CC3)cc2)CC1]

### Example 2c12. (3S)-3-((4-(4-((1-(4-(1-(1-(4-(6-aminopyridin-3-yl)-1-methyl-1H-pyrrolo[2,3-b] pyridin-2-yl)-ethyl)piperidin-4-yl)benzoyl)piperidin-4-yl)methyl)piperazin-1-yl)-3-fluorophenyl)amino)piperidine-2,6-dione

Example 2c12 (37 mg) prepared similarly to Example 2b1 from Ints. 2C9/2C11 (48 mg/60 mg, HCl salt). The absolute configuration at the glutarimide stereocenter was not determined.

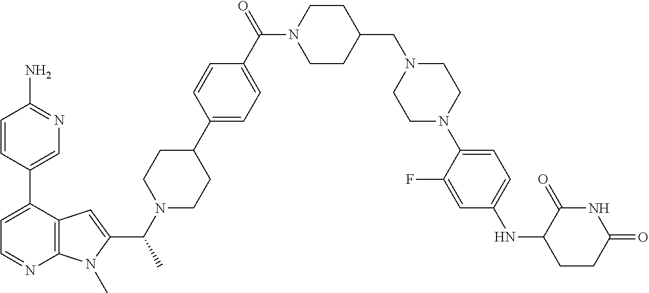 [SMILES: C[C@H](c1cc2c(-c3ccc(N)nc3)ccnc2n1C)N1CCC(c2ccc(C(=O)N3CCC(CN4CCN(c5ccc(NC6CCC(=O)NC6=O)cc5F)CC4)CC3)cc2)CC1]

### Example 2c13. 3-((4-(4-((1-(4-(1-((R)-1-(4-(6-aminopyridin-3-yl)-1-methyl-1H-pyrrolo[2,3-b] pyridin-2-yl)ethyl)-piperidin-4-yl)benzoyl)piperidin-4-yl)methyl)piperazin-1-yl)-3-fluorophenyl)amino)piperidine-2,6-dione

Example 2c13 (25 mg) was prepared similarly to Example 2c14 from Ints. 2C91/3E83′ (39 mg, 60 mg).

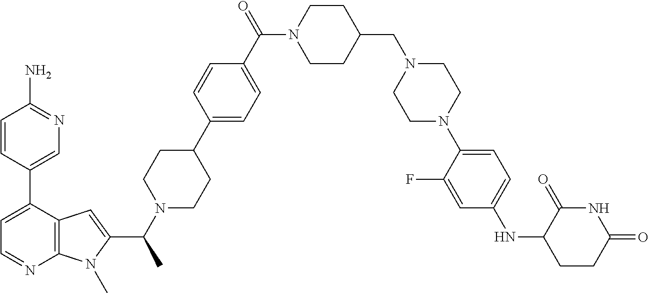 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5F)CC4)CC3)cc2)CC1]

### Example 2c14. 3-((4-(4-((1-(4-(1-((S)-1-(4-(6-aminopyridin-3-yl)-1-methyl-1H-pyrrolo[2,3-b] pyridin-2-yl)ethyl)-piperidin-4-yl)benzoyl)piperidin-4-yl)methyl)piperazin-1-yl)-3-fluorophenyl)amino)piperidine-2,6-dione

Ints. 3E83/2C91 (2.5 g/3.1 g, HCl salts) and HATU (2.5 g) were stirred ON in DMF/DIPEA (3.2:1, 26 mL) and diluted with water to precipitate a solid. This material was purified by FC (DCM/MeOH 93:7 to 9:1) to afford Example 2c14 (0.80 g).

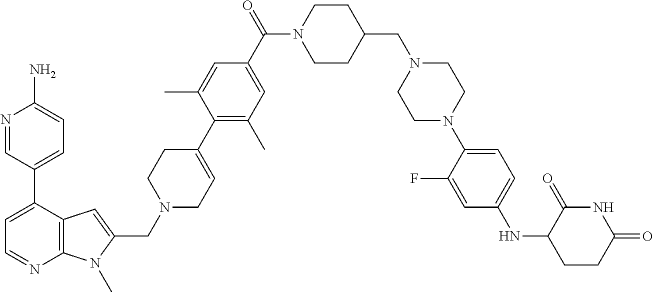 [SMILES: Cc1cc(C(=O)N2CCC(CN3CCN(c4ccc(NC5CCC(=O)NC5=O)cc4F)CC3)CC2)cc(C)c1C1=CCN(Cc2cc3c(-c4ccc(N)nc4)ccnc3n2C)CC1]

### Example 2c15. 3-((4-(4-((1-(4-(1-((4-(6-aminopyridin-3-yl)-1-methyl-1H-pyrrolo[2,3-b]pyridin-2-yl)methyl)-1,2,3,6-tetrahydropyridin-4-yl)-3,5-dimethylbenzoyl)piperidin-4-yl)methyl)piperazin-1-yl)-3-fluorophenyl)amino)piperidine-2,6-dione

Int. 2C18 (21 mg) was stirred 1 h in DCM/TFA (3:2, 0.5 mL) and concentrated. The OL (DCM/sat. aq. NaHCO₃) was concentrated and was purified by FC (DCM/MeOH 1:0 to 96:4) to give Example 2c15 (16 mg).

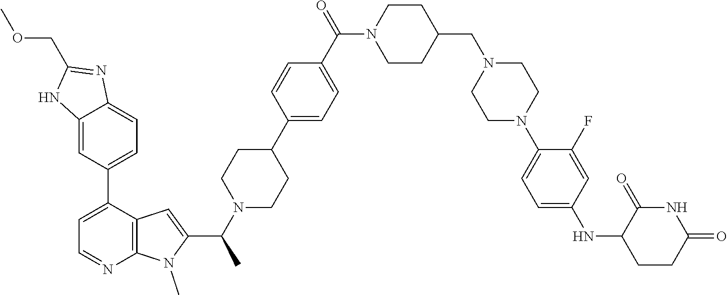 [SMILES: COCc1nc2ccc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)cc2[nH]1]

### Example 2c16. 3-((3-fluoro-4-(4-((1-(4-(1-((S)-1-(4-(2-(methoxymethyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione

Example 2c16 (48 mg) prepared similarly to Example 2b1 from Ints. 3E120/2C15 (0.30 g/0.38 g, HCl salt) after HPLC purification.

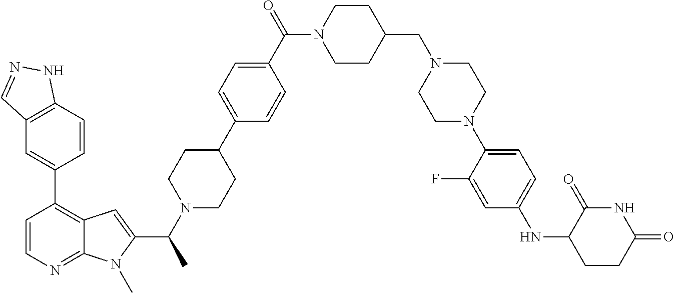 [SMILES: C[C@@H](c1cc2c(-c3ccc4[nH]ncc4c3)ccnc2n1C)N1CCC(c2ccc(C(=O)N3CCC(CN4CCN(c5ccc(NC6CCC(=O)NC6=O)cc5F)CC4)CC3)cc2)CC1]

### Example 2c17. 3-((4-(4-((1-(4-(1-((S)-1-(4-(1H-indazol-5-yl)-1-methyl-1H-pyrrolo[2,3-b] pyridin-2-yl)-ethyl)piperidin-4-yl)benzoyl)piperidin-4-yl)methyl)piperazin-1-yl)-3-fluorophenyl)amino)piperidine-2,6-dione

Example 2c17 (89 mg) prepared similarly to Example 2c15 from Int. 2C19 (0.19 g).

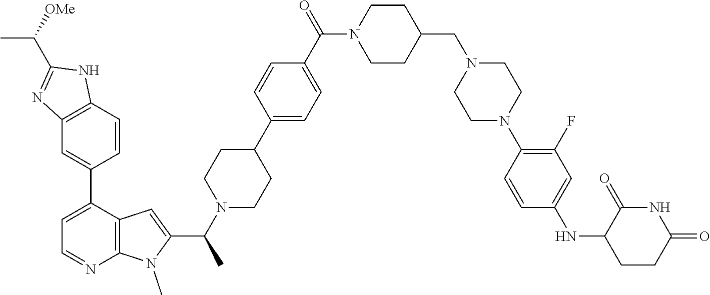 [SMILES: CO[C@@H](C)c1nc2cc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)ccc2[nH]1]

### Example 2c18. 3-((3-fluoro-4-(4-((1-(4-(1-((S)-1-(4-(2-((S)-1-methoxyethyl)-1H-benzo[d] imidazol-5-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione

Int. 2C30/2C31 (0.30 g) was stirred 3 h in DCM/4M HCl in dioxane (3:2, 5 mL) at 0° C. to RT. The residue after concentration was HPLC-purified to give Example 2c18 (32 mg).

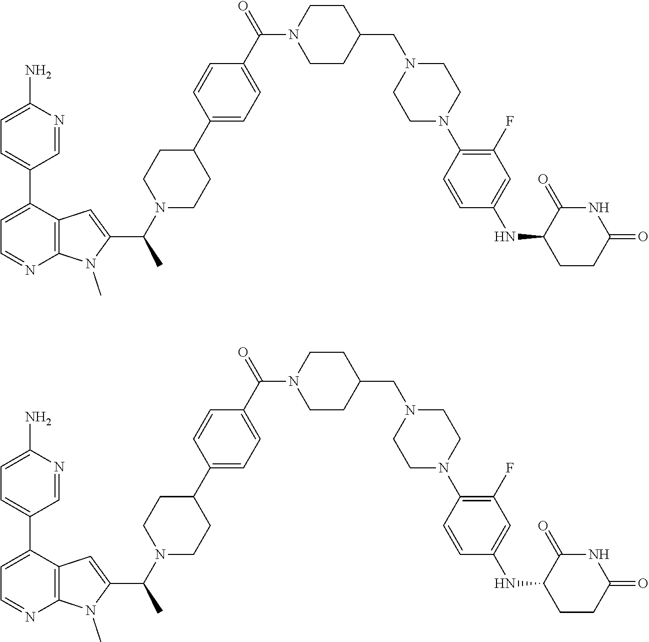 [SMILES: C[C@@H](c1cc2c(-c3ccc(N)nc3)ccnc2n1C)N1CCC(c2ccc(C(=O)N3CCC(CN4CCN(c5ccc(N[C@@H]6CCC(=O)NC6=O)cc5F)CC4)CC3)cc2)CC1 ; C[C@@H](c1cc2c(-c3ccc(N)nc3)ccnc2n1C)N1CCC(c2ccc(C(=O)N3CCC(CN4CCN(c5ccc(N[C@H]6CCC(=O)NC6=O)cc5F)CC4)CC3)cc2)CC1]

### Example 2c19 and 2c20. (S)-3-((4-(4-((1-(4-(1-((S)-1-(4-(6-aminopyridin-3-yl)-1-methyl-1H-pyrrolo[2,3-b]pyridin-2-yl)ethyl)piperidin-4-yl)benzoyl)piperidin-4-yl)methyl)piperazin-1-yl)-3-fluorophenyl)-amino)-piperidine-2,6-dione and (R)-3-((4-(4-((1-(4-(1-((S)-1-(4-(6-aminopyridin-3-yl)-1-methyl-1H-pyrrolo[2,3-b]pyridin-2-yl)ethyl)-piperidin-4-yl)benzoyl)piperidin-4-yl)methyl)piperazin-1-yl)-3-fluorophenyl)amino)-piperidine-2,6-dione

Example 2c14 (0.10 g) was separated by HPLC using a Chiralpak IE 4.6×250 mm, 5 micron column with an eluent of MeOH/DCM (1:1) to give Examples 2c19 (12 mg, first eluting peak) and 2c20 (10 mg, second eluting peak). The absolute configurations at the glutarimide stereocenters were not determined.

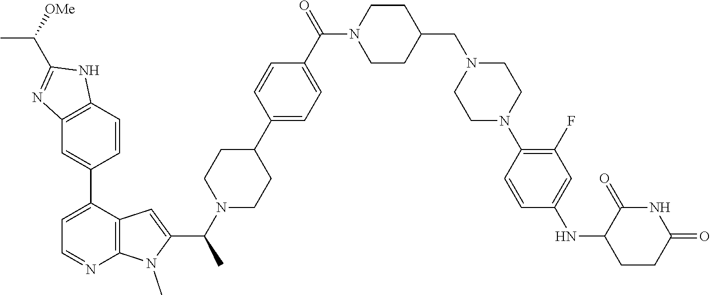 [SMILES: CO[C@@H](C)c1nc2cc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)ccc2[nH]1]

### Example 2c21. 3-((3-fluoro-4-(4-((1-(4-(1-((S)-1-(4-(2-((R)-1-methoxyethyl)-1H-benzo[d] imidazol-5-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione

Example 2c21 (15 mg) was prepared similarly to Example 2c18 from Int. 2C32/2C33 (0.15 g).

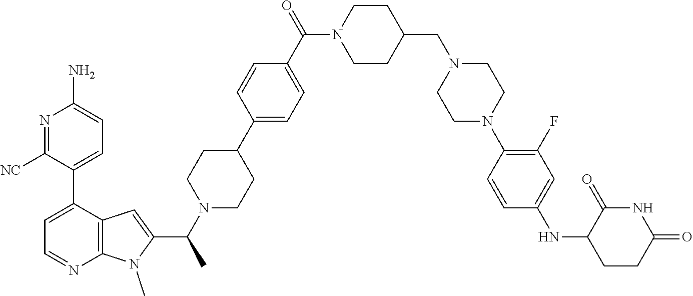 [SMILES: C[C@@H](c1cc2c(-c3ccc(N)nc3C#N)ccnc2n1C)N1CCC(c2ccc(C(=O)N3CCC(CN4CCN(c5ccc(NC6CCC(=O)NC6=O)cc5F)CC4)CC3)cc2)CC1]

### Example 2c22. 6-amino-3-(2-((1S)-1-(4-(4-(4-((4-(4-((2,6-dioxopiperidin-3-yl)amino)-2-fluorophenyl)-piperazin-1-yl)methyl)piperidine-1-carbonyl)phenyl) piperidin-1-yl)ethyl)-1-methyl-1H-pyrrolo[2,3-b]pyridin-4-yl) picolinonitrile

Int. 2C37 (0.10 g), 6-amino-3-(4,4,5,5-tetramethyl-1,3,2-dioxaborolan-2-yl) picolinonitrile (36 mg), K₃PO₄ (64 mg), and Ruphos Pd-G2 (9 mg) were degassed in dioxane/water (100:1, 20.2 mL) and stirred 1 h at 100° C. under MW conditions. Filtration, concentration, and HPLC-purified gave Example 2c22 (20 mg).

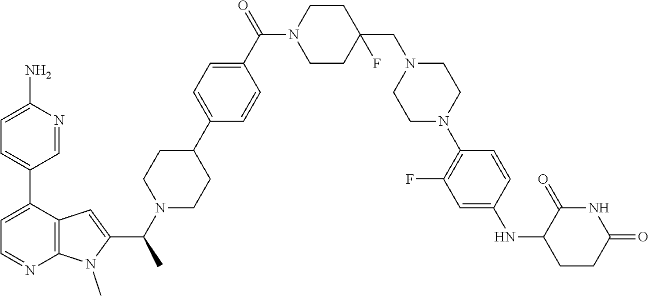 [SMILES: C[C@@H](c1cc2c(-c3ccc(N)nc3)ccnc2n1C)N1CCC(c2ccc(C(=O)N3CCC(F)(CN4CCN(c5ccc(NC6CCC(=O)NC6=O)cc5F)CC4)CC3)cc2)CC1]

### Example 2c23. 3-((4-(4-((1-(4-(1-((S)-1-(4-(6-aminopyridin-3-yl)-1-methyl-1H-pyrrolo[2,3-b] pyridin-2-yl)ethyl)-piperidin-4-yl)benzoyl)-4-fluoropiperidin-4-yl)methyl)piperazin-1-yl)-3-fluorophenyl)amino)-piperidine-2,6-dione

Example 2c23 (28 mg) prepared similarly to Example 2b1 from Ints. 3E83/2C49 (0.16 g/0.21 g, HCl salt).

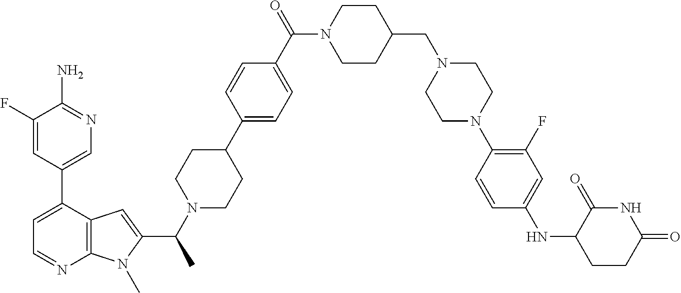 [SMILES: C[C@@H](c1cc2c(-c3cnc(N)c(F)c3)ccnc2n1C)N1CCC(c2ccc(C(=O)N3CCC(CN4CCN(c5ccc(NC6CCC(=O)NC6=O)cc5F)CC4)CC3)cc2)CC1]

### Example 2c24. 3-((4-(4-((1-(4-(1-((S)-1-(4-(6-amino-5-fluoropyridin-3-yl)-1-methyl-1H-pyrrolo[2,3-b]pyridin-2-yl)ethyl)piperidin-4-yl)benzoyl)piperidin-4-yl)methyl)piperazin-1-yl)-3-fluorophenyl)-amino)piperidine-2,6-dione

Example 2c24 (12 mg) prepared similarly to Example 2c22 from Int. 2C37 (0.15 g) and 3-fluoro-5-(4,4,5,5-tetramethyl-1,3,2-dioxaborolan-2-yl)pyridin-2-amine (52 mg).

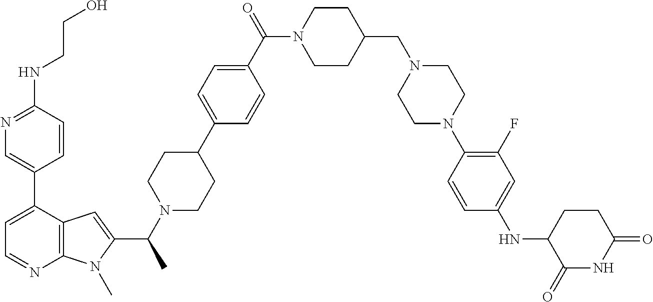 [SMILES: C[C@@H](c1cc2c(-c3ccc(NCCO)nc3)ccnc2n1C)N1CCC(c2ccc(C(=O)N3CCC(CN4CCN(c5ccc(NC6CCC(=O)NC6=O)cc5F)CC4)CC3)cc2)CC1]

### Example 2c26. 3-((3-fluoro-4-(4-((1-(4-(1-((S)-1-(4-(6-((2-hydroxyethyl)amino)pyridin-3-yl)-1-methyl-1H-pyrrolo[2,3-b]pyridin-2-yl)ethyl)piperidin-4-yl)benzoyl)piperidin-4-yl)methyl)piperazin-1-yl)phenyl)-amino)piperidine-2,6-dione

Example 2c26 (14 mg) prepared similarly to Example 2c22 from Int. 2C37 (0.18 g) and 2-((5-(4,4,5,5-tetramethyl-1,3,2-dioxaborolan-2-yl)pyridin-2-yl)amino) ethan-1-ol (0.12 g) using dioxane/water (9:1, 2 mL), K₃PO₄ (69 mg), and Ruphos Pd-G2 (17 mg).

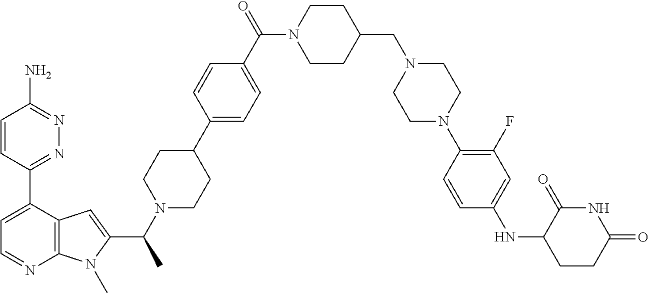 [SMILES: C[C@@H](c1cc2c(-c3ccc(N)nn3)ccnc2n1C)N1CCC(c2ccc(C(=O)N3CCC(CN4CCN(c5ccc(NC6CCC(=O)NC6=O)cc5F)CC4)CC3)cc2)CC1]

### Example 2c27. 3-((4-(4-((1-(4-(1-((S)-1-(4-(6-aminopyridazin-3-yl)-1-methyl-1H-pyrrolo[2,3-b] pyridin-2-yl)ethyl)piperidin-4-yl)benzoyl)piperidin-4-yl)methyl)piperazin-1-yl)-3-fluorophenyl)amino)piperidine-2,6-dione

Example 2c27 (26 mg) prepared similarly to Example 2b1 from Ints. 3E184/2C15 (0.10 g, HCl salt/82 mg, TFA salt) using DMF (5 mL), DIPEA (0.2 mL), and HATU (0.12 g).

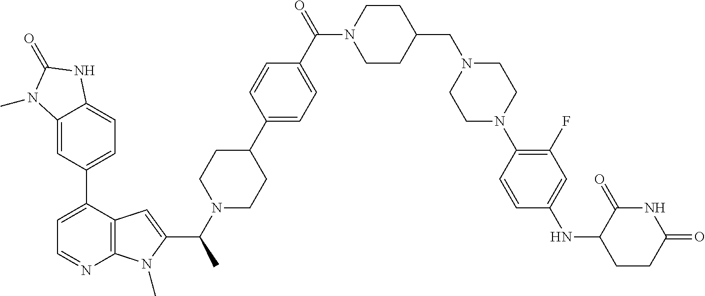 [SMILES: C[C@@H](c1cc2c(-c3ccc4[nH]c(=O)n(C)c4c3)ccnc2n1C)N1CCC(c2ccc(C(=O)N3CCC(CN4CCN(c5ccc(NC6CCC(=O)NC6=O)cc5F)CC4)CC3)cc2)CC1]

### Example 2c28. 3-((3-fluoro-4-(4-((1-(4-(1-((S)-1-(1-methyl-4-(3-methyl-2-oxo-2,3-dihydro-1H-benzo[d] imidazol-5-yl)-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione

Example 2c28 (19 mg) prepared similarly to Example 2b1 from Ints. 3E165/2C15 (0.10 g/0.13 g, HCl salt) using DMF (3 mL), DIPEA (0.4 mL), and HATU (0.11 g).

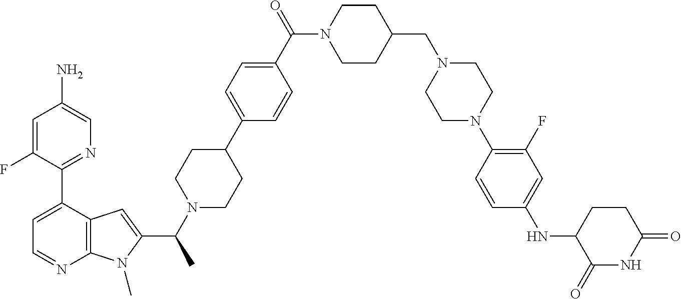 [SMILES: C[C@@H](c1cc2c(-c3ncc(N)cc3F)ccnc2n1C)N1CCC(c2ccc(C(=O)N3CCC(CN4CCN(c5ccc(NC6CCC(=O)NC6=O)cc5F)CC4)CC3)cc2)CC1]

### Example 2c30. 3-((4-(4-((1-(4-(1-((S)-1-(4-(5-amino-3-fluoropyridin-2-yl)-1-methyl-1H-pyrrolo[2,3-b]pyridin-2-yl)ethyl)piperidin-4-yl)benzoyl)piperidin-4-yl)methyl)piperazin-1-yl)-3-fluorophenyl)-amino)piperidine-2,6-dione

Example 2c30 (61 mg) prepared similarly to Example 2b1 from Ints. 3E168/2C15 (80 mg/0.14 g, HCl salt) using DMF (2 mL), DIPEA (0.3 mL), and HATU (96 mg).

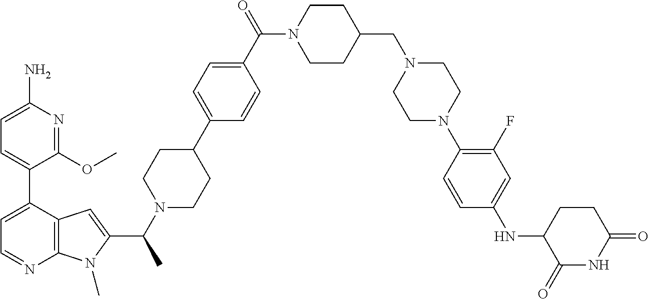 [SMILES: COc1nc(N)ccc1-c1ccnc2c1cc([C@H](C)N1CCC(c3ccc(C(=O)N4CCC(CN5CCN(c6ccc(NC7CCC(=O)NC7=O)cc6F)CC5)CC4)cc3)CC1)n2C]

### Example 2c32. 3-((4-(4-((1-(4-(1-((S)-1-(4-(6-amino-2-methoxypyridin-3-yl)-1-methyl-1H-pyrrolo[2,3-b]pyridin-2-yl)ethyl)piperidin-4-yl)benzoyl)piperidin-4-yl)methyl)piperazin-1-yl)-3-fluorophenyl)amino)-piperidine-2,6-dione

Example 2c32 (29 mg) prepared similarly to Example 2b1 from Ints. 3E175/2C15 (80 mg/0.17 g, HCl salt) using DMF (5 mL), DIPEA (0.2 mL), and HATU (94 mg).

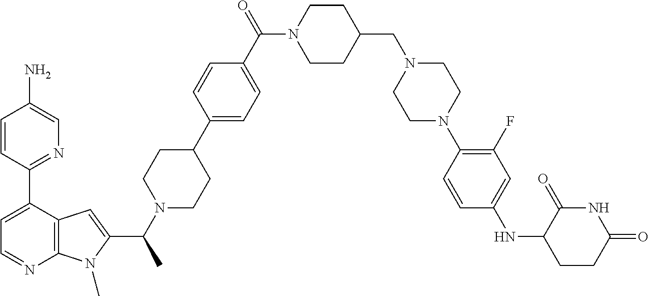 [SMILES: C[C@@H](c1cc2c(-c3ccc(N)cn3)ccnc2n1C)N1CCC(c2ccc(C(=O)N3CCC(CN4CCN(c5ccc(NC6CCC(=O)NC6=O)cc5F)CC4)CC3)cc2)CC1]

### Example 2c33. 3-((4-(4-((1-(4-(1-((S)-1-(4-(5-aminopyridin-2-yl)-1-methyl-1H-pyrrolo[2,3-b] pyridin-2-yl)ethyl)-piperidin-4-yl)benzoyl)piperidin-4-yl)methyl)piperazin-1-yl)-3-fluorophenyl)amino)piperidine-2,6-dione

Example 2c33 (19 mg, TFA salt) prepared similarly to Example 2c32 from Ints. 3E198/2C15 (80 mg/0.12 g, HCl salt).

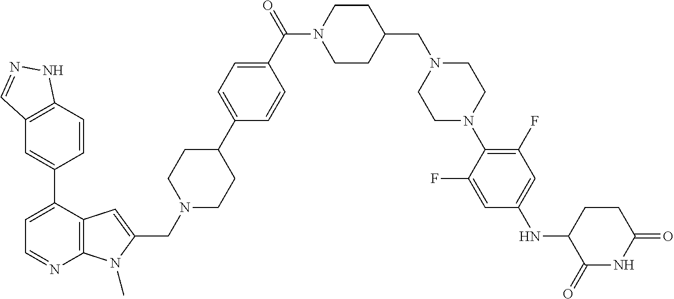 [SMILES: Cn1c(CN2CCC(c3ccc(C(=O)N4CCC(CN5CCN(c6c(F)cc(NC7CCC(=O)NC7=O)cc6F)CC5)CC4)cc3)CC2)cc2c(-c3ccc4[nH]ncc4c3)ccnc21]

### Example 2c34. 3-((4-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)-3,5-difluorophenyl)amino)piperidine-2,6-dione

Example 2c34 (15 mg) prepared similarly to Example 2b1 from Ints. 3A1/2I24 (35 mg, TFA salt/23 mg, HCl salt).

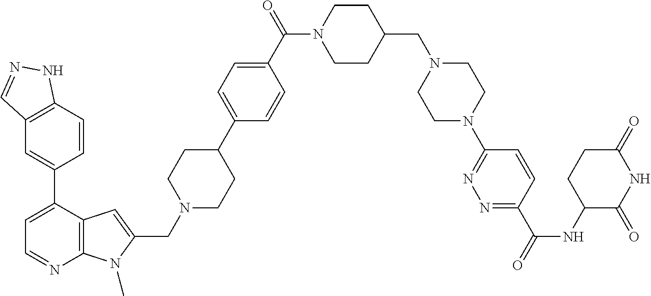 [SMILES: Cn1c(CN2CCC(c3ccc(C(=O)N4CCC(CN5CCN(c6ccc(C(=O)NC7CCC(=O)NC7=O)nn6)CC5)CC4)cc3)CC2)cc2c(-c3ccc4[nH]ncc4c3)ccnc21]

### Example 2d1. 6-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)-N-(2,6-dioxopiperidin-3-yl) pyridazine-3-carboxamide

Example 2d1 (14 mg) prepared similarly to Example 2b1 from Ints. 3A1/2D1 (25 mg/26 mg, HCl salt).

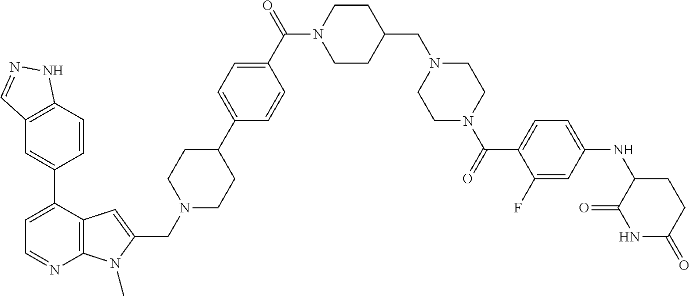 [SMILES: Cn1c(CN2CCC(c3ccc(C(=O)N4CCC(CN5CCN(C(=O)c6ccc(NC7CCC(=O)NC7=O)cc6F)CC5)CC4)cc3)CC2)cc2c(-c3ccc4[nH]ncc4c3)ccnc21]

### Example 2e1. 3-((4-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazine-1-carbonyl)-3-fluorophenyl)amino)piperidine-2,6-dione

Example 2e1 (14 mg) prepared similarly to Example 2b1 from Ints. 3A1/2E2 (33 mg/30 mg, TFA salt).

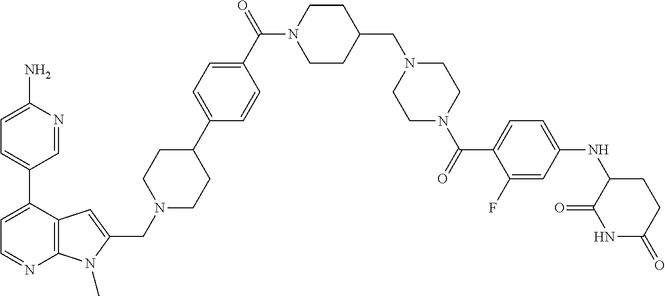 [SMILES: Cn1c(CN2CCC(c3ccc(C(=O)N4CCC(CN5CCN(C(=O)c6ccc(NC7CCC(=O)NC7=O)cc6F)CC5)CC4)cc3)CC2)cc2c(-c3ccc(N)nc3)ccnc21]

### Example 2e2. 3-((4-(4-((1-(4-(1-((4-(6-aminopyridin-3-yl)-1-methyl-1H-pyrrolo[2,3-b]pyridin-2-yl)methyl)-piperidin-4-yl)benzoyl)piperidin-4-yl)methyl)piperazine-1-carbonyl)-3-fluorophenyl)amino)piperidine-2,6-dione

Example 2e2 (22 mg) prepared similarly to Example 2c15 from Int. 2E1 (33 mg).

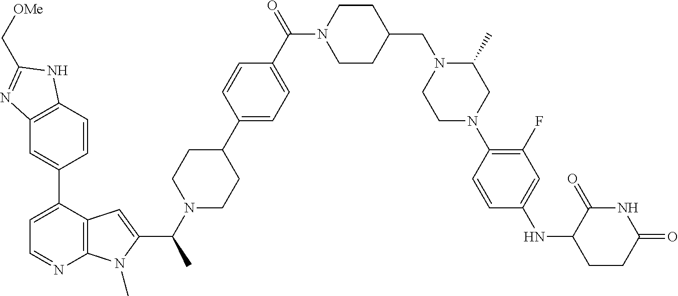 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8F)C[C@H]7C)CC6)cc5)CC3)n4C)ccc2[nH]1]

### Example 2f1. 3-((3-fluoro-4-((R)-4-((1-(4-(1-((S)-1-(4-(2-(methoxymethyl)-1H-benzo[d] imidazol-5-yl)-1-methyl-1H-pyrrolo[2,3-b]pyridin-2-yl)ethyl)piperidin-4-yl)benzoyl)piperidin-4-yl)methyl)-3-methylpiperazin-1-yl)phenyl)amino)piperidine-2,6-dione

Example 2f1 (14 mg) prepared similarly to Example 2b1 from HCl salts of Ints. 2F1/2F10 (0.10 g/81 mg).

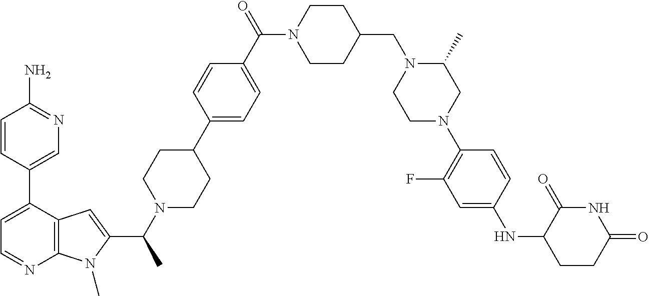 [SMILES: C[C@@H]1CN(c2ccc(NC3CCC(=O)NC3=O)cc2F)CCN1CC1CCN(C(=O)c2ccc(C3CCN([C@@H](C)c4cc5c(-c6ccc(N)nc6)ccnc5n4C)CC3)cc2)CC1]

### Example 2f2. 3-((4-((R)-4-((1-(4-(1-((S)-1-(4-(6-aminopyridin-3-yl)-1-methyl-1H-pyrrolo[2,3-b] pyridin-2-yl)ethyl)piperidin-4-yl)benzoyl)piperidin-4-yl)methyl)-3-methylpiperazin-1-yl)-3-fluorophenyl)amino)-piperidine-2,6-dione

Example 2f2 (29 mg) prepared similarly to Example 2b1 from HCl salts of Ints. 3E83/2F10 (0.10 g/82 mg).

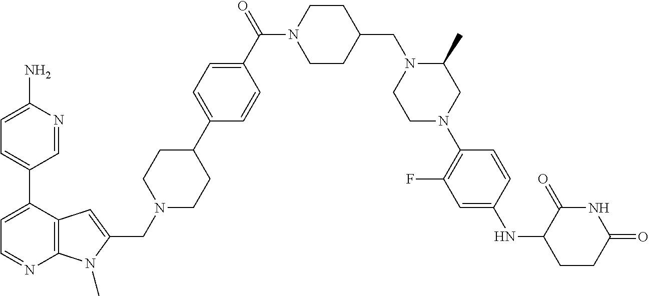 [SMILES: C[C@H]1CN(c2ccc(NC3CCC(=O)NC3=O)cc2F)CCN1CC1CCN(C(=O)c2ccc(C3CCN(Cc4cc5c(-c6ccc(N)nc6)ccnc5n4C)CC3)cc2)CC1]

### Example 2f3. 3-((4-((S)-4-((1-(4-(1-((4-(6-aminopyridin-3-yl)-1-methyl-1H-pyrrolo[2,3-b] pyridin-2-yl)methyl)piperidin-4-yl)benzoyl)piperidin-4-yl)methyl)-3-methylpiperazin-1-yl)-3-fluorophenyl)amino)-piperidine-2,6-dione

Int. 2F20 (39 mg) was stirred 2.5 h in DCM/TFA (1 mL/1 mL). The residue after concentration was HPLC-purified to give Example 2f3 (16 mg).

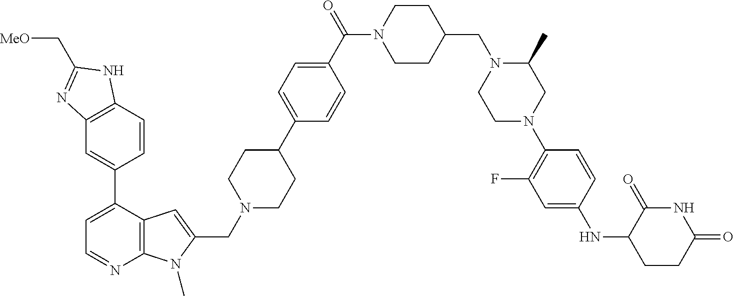 [SMILES: COCc1nc2cc(-c3ccnc4c3cc(CN3CCC(c5ccc(C(=O)N6CCC(CN7CCN(c8ccc(NC9CCC(=O)NC9=O)cc8F)C[C@@H]7C)CC6)cc5)CC3)n4C)ccc2[nH]1]

### Example 2f4. 3-((3-fluoro-4-((S)-4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)-3-methylpiperazin-1-yl)phenyl)-amino)piperidine-2,6-dione

Example 2f4 (17 mg) prepared similarly to Example 2b1 from HCl salts of Ints. 3E261/2F21 (32 mg/23 mg).

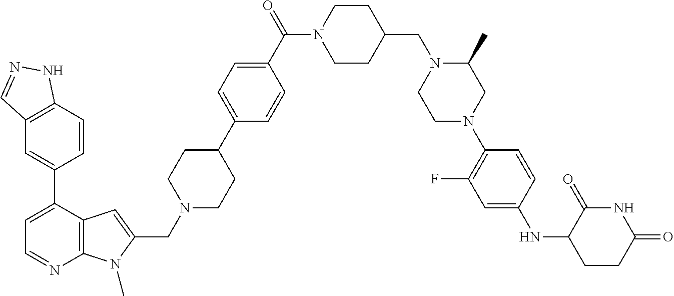 [SMILES: C[C@H]1CN(c2ccc(NC3CCC(=O)NC3=O)cc2F)CCN1CC1CCN(C(=O)c2ccc(C3CCN(Cc4cc5c(-c6ccc7[nH]ncc7c6)ccnc5n4C)CC3)cc2)CC1]

### Example 2f5. 3-((4-((S)-4-((1-(4-(1-((4-(1H-indazol-5-yl)-1-methyl-1H-pyrrolo[2,3-b]pyridin-2-yl)methyl)piperidin-4-yl)benzoyl)piperidin-4-yl)methyl)-3-methylpiperazin-1-yl)-3-fluorophenyl)amino)piperidine-2,6-dione

Example 2f5 (24 mg) prepared similarly to Example 2b1 from Ints. 3A1/2F21 (26 mg/23 mg, HCl salt).

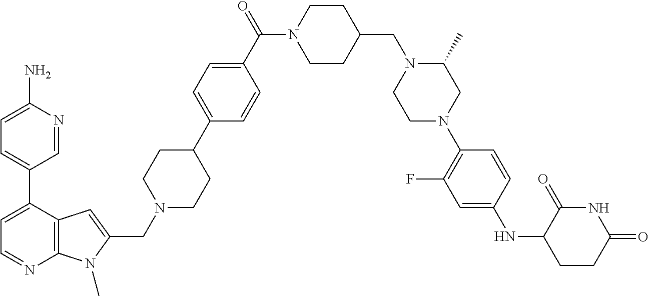 [SMILES: C[C@@H]1CN(c2ccc(NC3CCC(=O)NC3=O)cc2F)CCN1CC1CCN(C(=O)c2ccc(C3CCN(Cc4cc5c(-c6ccc(N)nc6)ccnc5n4C)CC3)cc2)CC1]

### Example 2f6. 3-((4-((R)-4-((1-(4-(1-((4-(6-aminopyridin-3-yl)-1-methyl-1H-pyrrolo[2,3-b] pyridin-2-yl)-methyl)-piperidin-4-yl)benzoyl)piperidin-4-yl)methyl)-3-methylpiperazin-1-yl)-3-fluorophenyl)amino)-piperidine-2,6-dione

Example 2f6 (5 mg) prepared similarly to Example 2f3 from Int. 2F27 (39 mg).

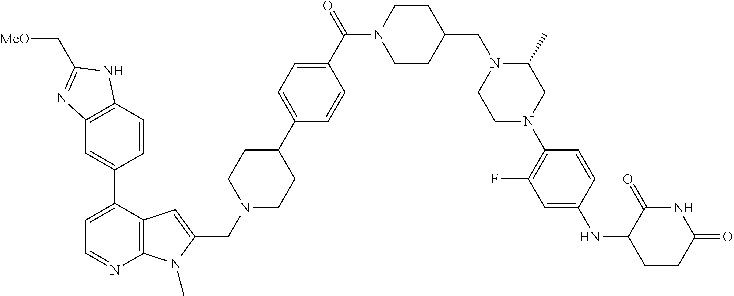 [SMILES: COCc1nc2cc(-c3ccnc4c3cc(CN3CCC(c5ccc(C(=O)N6CCC(CN7CCN(c8ccc(NC9CCC(=O)NC9=O)cc8F)C[C@H]7C)CC6)cc5)CC3)n4C)ccc2[nH]1]

### Example 2f7. 3-((3-fluoro-4-((R)-4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)-3-methylpiperazin-1-yl)phenyl)-amino)piperidine-2,6-dione

Example 2f7 (27 mg) prepared similarly to Example 2b1 from HCl salts of Ints. 3E261/2F10 (42 mg/30 mg).

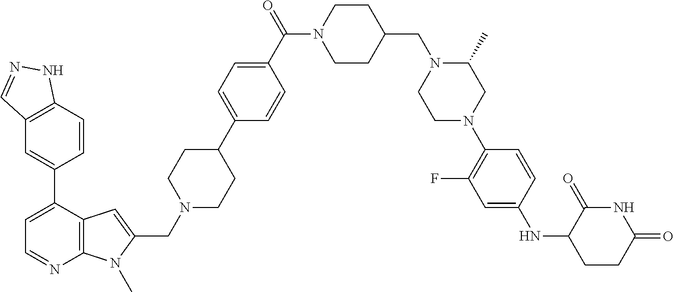 [SMILES: C[C@@H]1CN(c2ccc(NC3CCC(=O)NC3=O)cc2F)CCN1CC1CCN(C(=O)c2ccc(C3CCN(Cc4cc5c(-c6ccc7[nH]ncc7c6)ccnc5n4C)CC3)cc2)CC1]

### Example 2f8. 3-((4-((R)-4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)-3-methylpiperazin-1-yl)-3-fluorophenyl)amino)piperidine-2,6-dione

Example 2f8 (50 mg) prepared similarly to Example 2b1 from Ints. 3A1/2F10 (45 mg/42 mg, HCl salt).

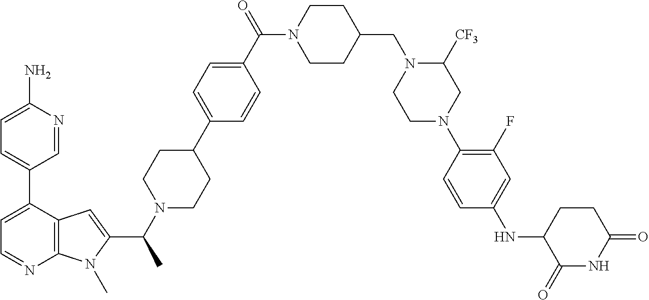 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5F)CC4C(F)(F)F)CC3)cc2)CC1]

### Example 2f9. 3-((4-(4-((1-(4-(1-((S)-1-(4-(6-aminopyridin-3-yl)-1-methyl-1H-pyrrolo[2,3-b] pyridin-2-yl)-ethyl)piperidin-4-yl)benzoyl)piperidin-4-yl)methyl)-3-(trifluoromethyl)-piperazin-1-yl)-3-fluorophenyl)-amino)piperidine-2,6-dione

Example 2f9 (45 mg) prepared similarly to Example 2b1 from HCl salts of Ints. 3E83/2F16 (0.17 g/0.26 g).

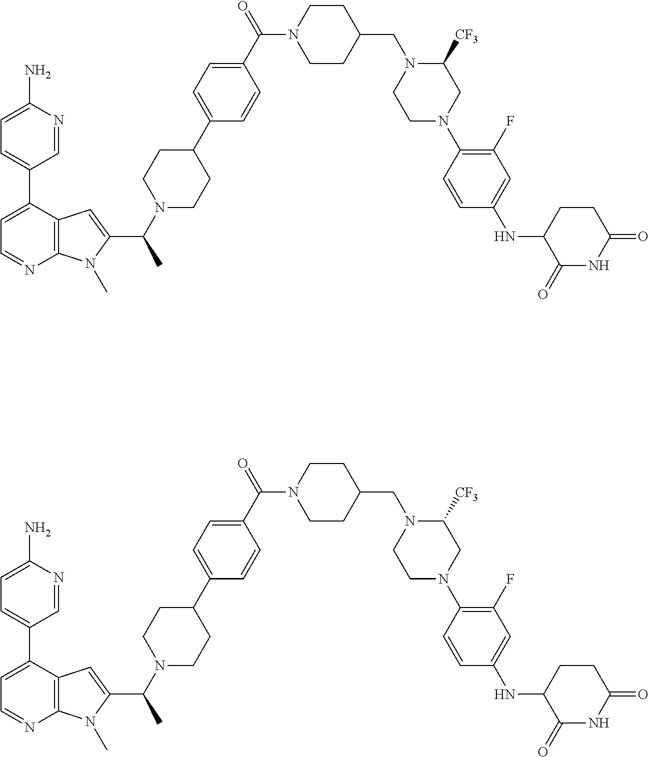 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5F)C[C@@H]4C(F)(F)F)CC3)cc2)CC1 ; C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5F)C[C@H]4C(F)(F)F)CC3)cc2)CC1]

### Examples 2f10 and 2f11. 3-((4-((S)-4-((1-(4-(1-((S)-1-(4-(6-aminopyridin-3-yl)-1-methyl-1H-pyrrolo[2,3-b]pyridin-2-yl)ethyl)piperidin-4-yl)benzoyl)piperidin-4-yl)methyl)-3-(trifluoromethyl)piperazin-1-yl)-3-fluorophenyl)-amino)piperidine-2,6-dione and 3-((4-((R)-4-((1-(4-(1-((S)-1-(4-(6-aminopyridin-3-yl)-1-methyl-1H-pyrrolo[2,3-b]pyridin-2-yl)ethyl)piperidin-4-yl)benzoyl)piperidin-4-yl)methyl)-3-(trifluoromethyl)piperazin-1-yl)-3-fluoro-phenyl)amino)piperidine-2,6-dione

Example 2f9 (33 mg) was separated by HPLC (Chiralpak IF 10×250 mm, 5 μm column, ACN: IPA: DCM (50:25:25). Flow rate: 3 mL/min) to give Examples 2f10 (9 mg, first eluting peak) and 2f11 (10 mg, second eluting peak). The absolute configurations at the piperazine stereocenters were not determined.

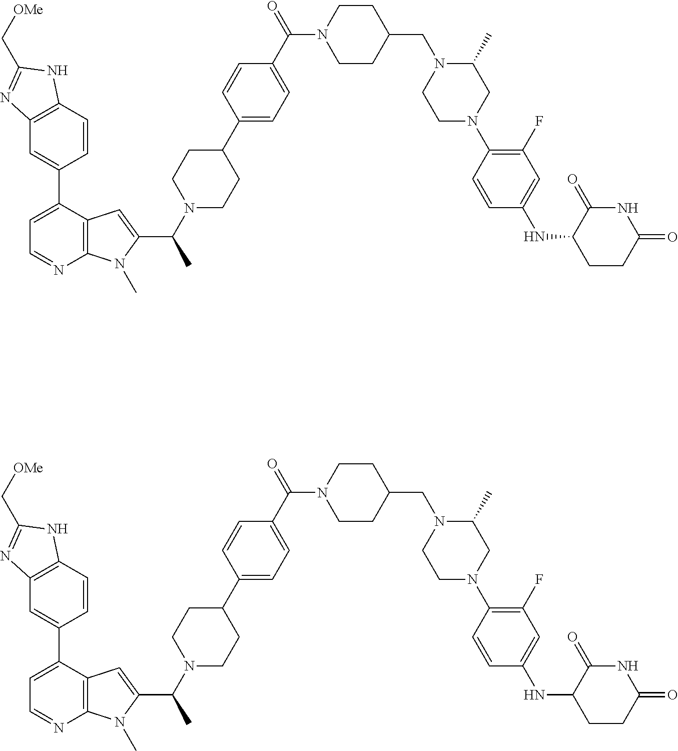 [SMILES: COCc1nc2cc(-c3ccnc4c3cc([C@H](C)N3CCC(c5ccc(C(=O)N6CCC(CN7CCN(c8ccc(N[C@H]9CCC(=O)NC9=O)cc8F)C[C@H]7C)CC6)cc5)CC3)n4C)ccc2[nH]1 ; COCc1nc2cc(-c3ccnc4c3cc([C@H](C)N3CCC(c5ccc(C(=O)N6CCC(CN7CCN(c8ccc(NC9CCC(=O)NC9=O)cc8F)C[C@H]7C)CC6)cc5)CC3)n4C)ccc2[nH]1]

### Examples 2f12 and 2f13. (S)-3-((3-fluoro-4-((R)-4-((1-(4-(1-((S)-1-(4-(2-(methoxymethyl)-1H-benzo[d]-imidazol-5-yl)-1-methyl-1H-pyrrolo[2,3-b]pyridin-2-yl)ethyl)piperidin-4-yl)benzoyl)piperidin-4-yl)-methyl)-3-methylpiperazin-1-yl)phenyl)amino)piperidine-2,6-dione and (R)-3-((3-fluoro-4-((R)-4-((1-(4-(1-((S)-1-(4-(2-(methoxymethyl)-1H-benzo[d]imidazol-5-yl)-1-methyl-1H-pyrrolo[2,3-b]pyridin-2-yl)-ethyl)piperidin-4-yl)benzoyl)piperidin-4-yl)methyl)-3-methylpiperazin-1-yl)phenyl)amino)piperidine-2,6-dione

Example 2f1 (0.40 g) was separated by HPLC using a Chiralpak IE 250×30 mm, 5 μm column and an eluent of ACN: IPA: DCM (35:35:30), a flow rate of 28 mL/min. at 40° C.) to give Example 2f12 (0.15 g, first eluting peak) and Example 2f13 (0.14 g, second eluting peak). The configurations of the chiral centers in the glutarimide rings were not determined.

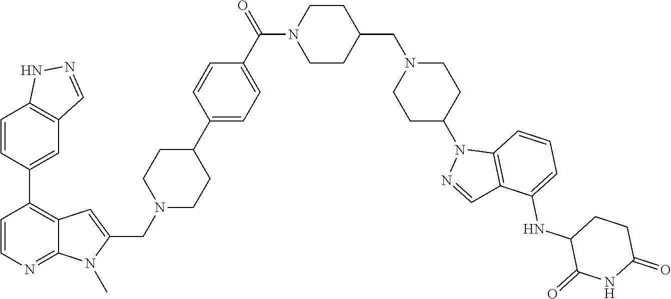 [SMILES: Cn1c(CN2CCC(c3ccc(C(=O)N4CCC(CN5CCC(n6ncc7c(NC8CCC(=O)NC8=O)cccc76)CC5)CC4)cc3)CC2)cc2c(-c3ccc4[nH]ncc4c3)ccnc21]

### Example 2g1. 3-((1-(1-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperidin-4-yl)-1H-indazol-4-yl)amino)piperidine-2,6-dione

TFA salts of Ints. 2G1 (32 mg) and 2G2 (26 mg) were stirred 0.5 h in DCM/DMF/DIPEA (4:1:0.06, 2.53 mL). STAB (31 mg) was added and stirring continued ON. The mixture was HPLC-purified to give Example 2g1 (2 mg).

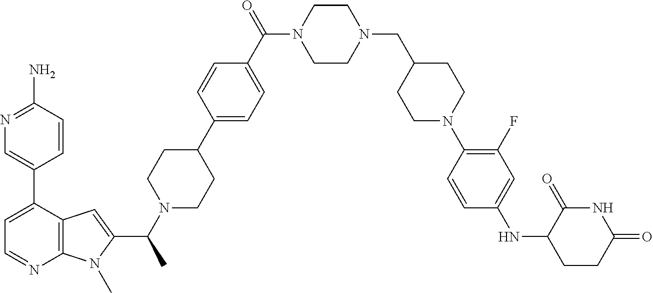 [SMILES: C[C@@H](c1cc2c(-c3ccc(N)nc3)ccnc2n1C)N1CCC(c2ccc(C(=O)N3CCN(CC4CCN(c5ccc(NC6CCC(=O)NC6=O)cc5F)CC4)CC3)cc2)CC1]

### Example 2g63. 3-((4-(4-((4-(4-(1-((S)-1-(4-(6-aminopyridin-3-yl)-1-methyl-1H-pyrrolo[2,3-b] pyridin-2-yl)ethyl)piperidin-4-yl)benzoyl) piperazin-1-yl)methyl)piperidin-1-yl)-3-fluorophenyl)amino)piperidine-2,6-dione

Example 2g63 (3 mg) was prepared similarly to Example 2c54 from Ints. 3E83/2G40 (23 mg/20 mg) using HATU (23 mg) instead of T3P.

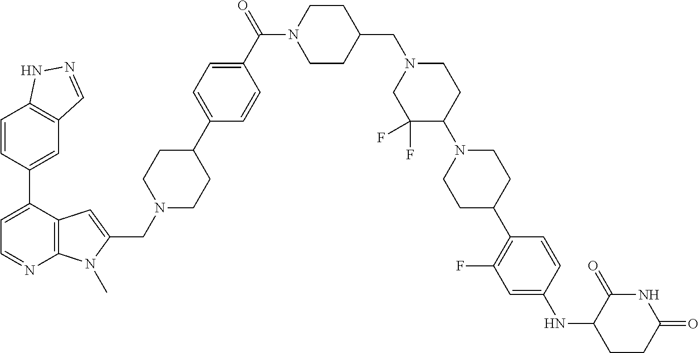 [SMILES: Cn1c(CN2CCC(c3ccc(C(=O)N4CCC(CN5CCC(N6CCC(c7ccc(NC8CCC(=O)NC8=O)cc7F)CC6)C(F)(F)C5)CC4)cc3)CC2)cc2c(-c3ccc4[nH]ncc4c3)ccnc21]

### Example 2h1. 3-((4-(1′-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)-3′, 3′-difluoro-[1,4′-bipiperidin]-4-yl)-3-fluorophenyl)-amino)piperidine-2,6-dione

Example 2h1 (14 mg) prepared similarly to Example 2f3 from Int. 2H1 (22 mg).

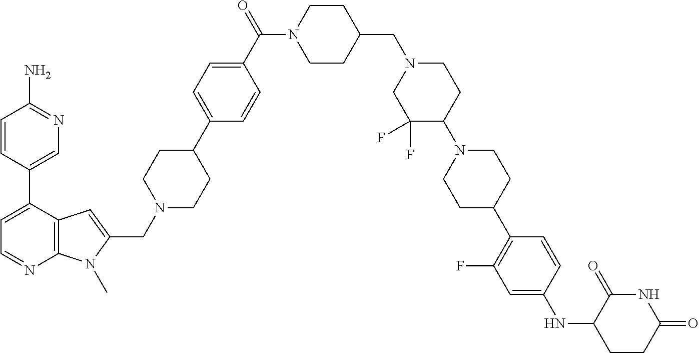 [SMILES: Cn1c(CN2CCC(c3ccc(C(=O)N4CCC(CN5CCC(N6CCC(c7ccc(NC8CCC(=O)NC8=O)cc7F)CC6)C(F)(F)C5)CC4)cc3)CC2)cc2c(-c3ccc(N)nc3)ccnc21]

### Example 2h2. 3-((4-(1′-((1-(4-(1-((4-(6-aminopyridin-3-yl)-1-methyl-1H-pyrrolo[2,3-b]pyridin-2-yl)-methyl)piperidin-4-yl)benzoyl)piperidin-4-yl)methyl)-3′, 3′-difluoro-[1,4′-bipiperidin]-4-yl)-3-fluoro-phenyl)amino)piperidine-2,6-dione

Example 2h2 (13 mg) prepared similarly to Example 2f3 from Int. 2H11 (33 mg).

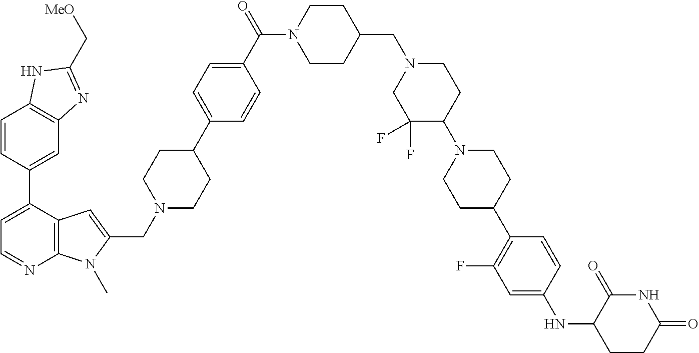 [SMILES: COCc1nc2cc(-c3ccnc4c3cc(CN3CCC(c5ccc(C(=O)N6CCC(CN7CCC(N8CCC(c9ccc(NC%10CCC(=O)NC%10=O)cc9F)CC8)C(F)(F)C7)CC6)cc5)CC3)n4C)ccc2[nH]1]

### Example 2h3. 3-((4-(3′, 3′-difluoro-1′-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)-[1,4′-bipiperidin]-4-yl)-3-fluorophenyl)amino)piperidine-2,6-dione

Example 2h3 (11 mg) prepared similarly to Example 2f3 from Int. 2H12/2H13 (19 mg).

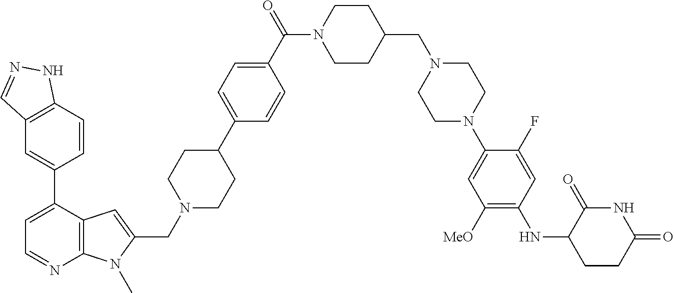 [SMILES: COc1cc(N2CCN(CC3CCN(C(=O)c4ccc(C5CCN(Cc6cc7c(-c8ccc9[nH]ncc9c8)ccnc7n6C)CC5)cc4)CC3)CC2)c(F)cc1NC1CCC(=O)NC1=O]

### Example 2i1. 3-((4-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)-5-fluoro-2-methoxyphenyl)amino)-piperidine-2,6-dione

Example 2i1 (16 mg) prepared similarly to Example 2b1 from Ints. 3A1/2I1 (34 mg, TFA salt/23 mg, HCl salt).

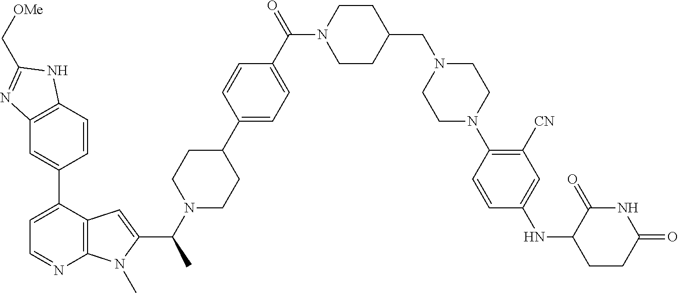 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8C#N)CC7)CC6)cc5)CC3)n4C)ccc2[nH]1]

### Example 2i2. 5-((2,6-dioxopiperidin-3-yl)amino)-2-(4-((1-(4-(1-((S)-1-(4-(2-(methoxymethyl)-1H-benzo[d]-imidazol-5-yl)-1-methyl-1H-pyrrolo[2,3-b]pyridin-2-yl)ethyl)piperidin-4-yl)benzoyl)piperidin-4-yl)methyl)-piperazin-1-yl)benzonitrile

Example 2i2 (23 mg) prepared similarly to Example 2c18 from Int. 215/216 (0.12 g).

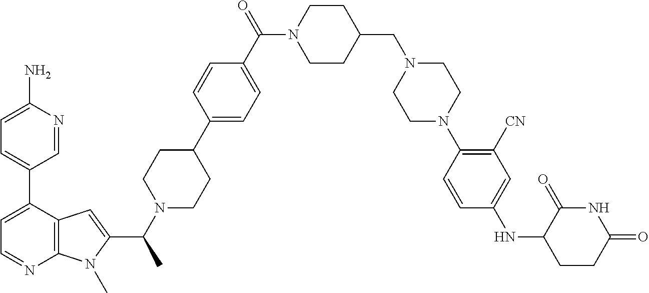 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5C#N)CC4)CC3)cc2)CC1]

### Example 2i3. 2-(4-((1-(4-(1-((S)-1-(4-(6-aminopyridin-3-yl)-1-methyl-1H-pyrrolo[2,3-b]pyridin-2-yl)-ethyl)-piperidin-4-yl)benzoyl)piperidin-4-yl)methyl)piperazin-1-yl)-5-((2,6-dioxopiperidin-3-yl)amino)-benzonitrile

Example 2i3 (22 mg) prepared similarly to Example 2c18 from Int. 2I17 (0.10 g).

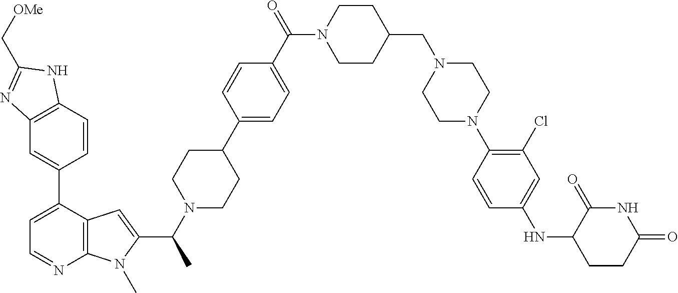 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8Cl)CC7)CC6)cc5)CC3)n4C)ccc2[nH]1]

### Example 2i4. 3-((3-chloro-4-(4-((1-(4-(1-((S)-1-(4-(2-(methoxymethyl)-1H-benzo[d]imidazol-5-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione

Example 2i4 (12 mg) prepared similarly to Example 2c18 from Int. 2I7/2I8 (0.17 g).

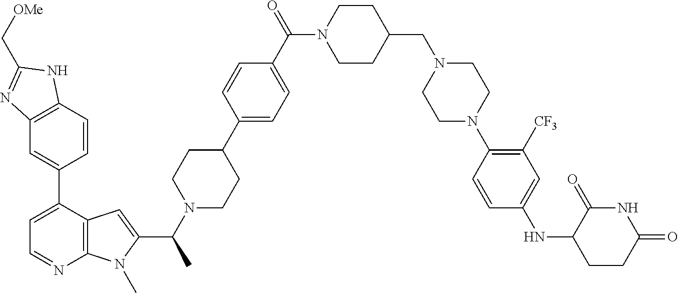 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8C(F)(F)F)CC7)CC6)cc5)CC3)n4C)ccc2[nH]1]

### Example 2i5. 3-((4-(4-((1-(4-(1-((S)-1-(4-(2-(methoxymethyl)-1H-benzo[d]imidazol-5-yl)-1-methyl-1H-pyrrolo[2,3-b]pyridin-2-yl)ethyl)piperidin-4-yl)benzoyl)piperidin-4-yl)methyl)piperazin-1-yl)-3-(trifluoromethyl)phenyl)amino)piperidine-2,6-dione

Example 2i5 (9 mg) prepared similarly to Example 2c18 from Int. 2I18/2I19 (0.55 g).

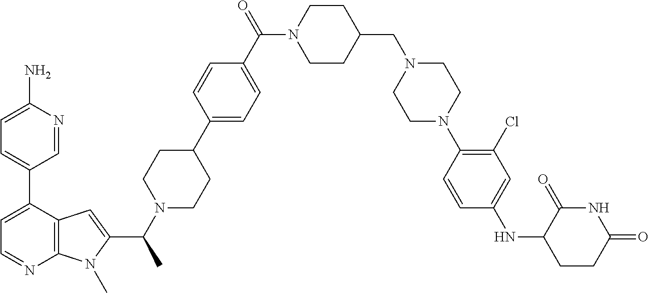 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5Cl)CC4)CC3)cc2)CC1]

### Example 2i6. 3-((4-(4-((1-(4-(1-((S)-1-(4-(6-aminopyridin-3-yl)-1-methyl-1H-pyrrolo[2,3-b] pyridin-2-yl)-ethyl)piperidin-4-yl)benzoyl)piperidin-4-yl)methyl)piperazin-1-yl)-3-chlorophenyl)amino)piperidine-2,6-dione

Example 2i6 (13 mg) prepared similarly to Example 2c18 from Int. 2I28 (0.13 g).

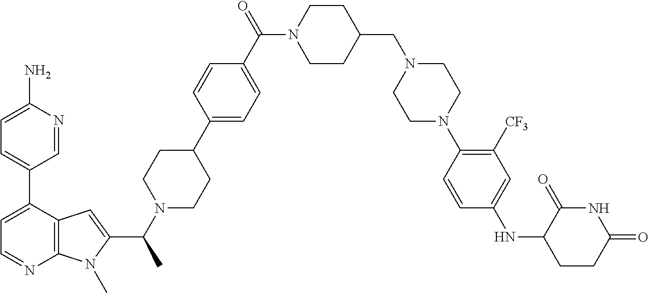 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5C(F)(F)F)CC4)CC3)cc2)CC1]

### Example 2i7. 3-((4-(4-((1-(4-(1-((S)-1-(4-(6-aminopyridin-3-yl)-1-methyl-1H-pyrrolo[2,3-b] pyridin-2-yl)-ethyl)-piperidin-4-yl)benzoyl)piperidin-4-yl)methyl)piperazin-1-yl)-3-(trifluoromethyl)phenyl)amino)-piperidine-2,6-dione

Example 2i7 (5 mg) prepared similarly to Example 2c18 from Int. 2I29 (0.31 g).

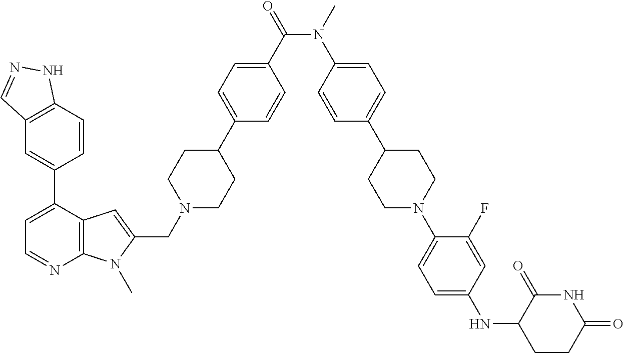 [SMILES: CN(C(=O)c1ccc(C2CCN(Cc3cc4c(-c5ccc6[nH]ncc6c5)ccnc4n3C)CC2)cc1)c1ccc(C2CCN(c3ccc(NC4CCC(=O)NC4=O)cc3F)CC2)cc1]

### Example 2j1. 4-(1-((4-(1H-indazol-5-yl)-1-methyl-1H-pyrrolo[2,3-b]pyridin-2-yl)methyl)piperidin-4-yl)-N-(4-(1-(4-((2,6-dioxopiperidin-3-yl)amino)-2-fluorophenyl) piperidin-4-yl)phenyl)-N-methylbenzamide

Example 2j1 (6 mg) prepared similarly to Example 2b1 from Ints. 3A1/2J1 (40 mg, TFA salt/22 mg).

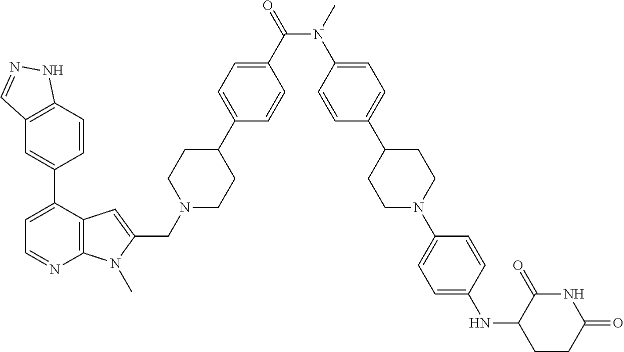 [SMILES: CN(C(=O)c1ccc(C2CCN(Cc3cc4c(-c5ccc6[nH]ncc6c5)ccnc4n3C)CC2)cc1)c1ccc(C2CCN(c3ccc(NC4CCC(=O)NC4=O)cc3)CC2)cc1]

### Example 2j2. 4-(1-((4-(1H-indazol-5-yl)-1-methyl-1H-pyrrolo[2,3-b]pyridin-2-yl)methyl)piperidin-4-yl)-N-(4-(1-(4-((2,6-dioxopiperidin-3-yl)amino)phenyl) piperidin-4-yl)phenyl)-N-methylbenzamide

Example 2j2 (9 mg) prepared similarly to Example 2b1 from Ints. 3A1/2J9 (35 mg, TFA salt/28 mg).

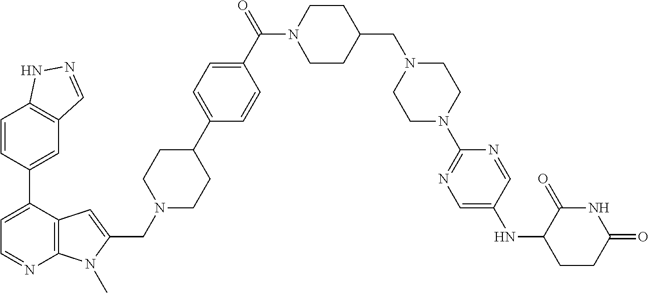 [SMILES: Cn1c(CN2CCC(c3ccc(C(=O)N4CCC(CN5CCN(c6ncc(NC7CCC(=O)NC7=O)cn6)CC5)CC4)cc3)CC2)cc2c(-c3ccc4[nH]ncc4c3)ccnc21]

### Example 2k1. 3-((2-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)pyrimidin-5-yl)amino)piperidine-2,6-dione

Example 2k1 (50 mg) prepared similarly to Example 2b1 from HCl salts of Ints. 3A1/2K1 (30 mg/30 mg).

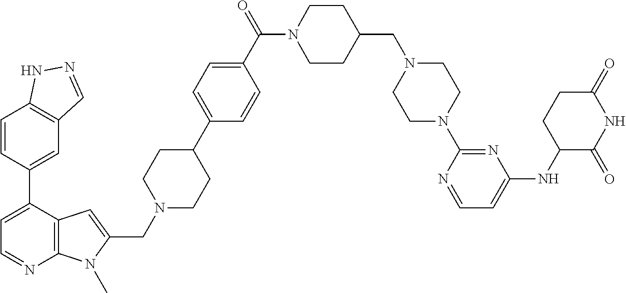 [SMILES: Cn1c(CN2CCC(c3ccc(C(=O)N4CCC(CN5CCN(c6nccc(NC7CCC(=O)NC7=O)n6)CC5)CC4)cc3)CC2)cc2c(-c3ccc4[nH]ncc4c3)ccnc21]

### Example 2k2. 3-((2-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)pyrimidin-4-yl)amino)piperidine-2,6-dione

Example 2k2 (15 mg) prepared similarly to Example 2b1 from Ints. 3A1/2K5 (25 mg, HCl salt/19 mg, TFA salt).

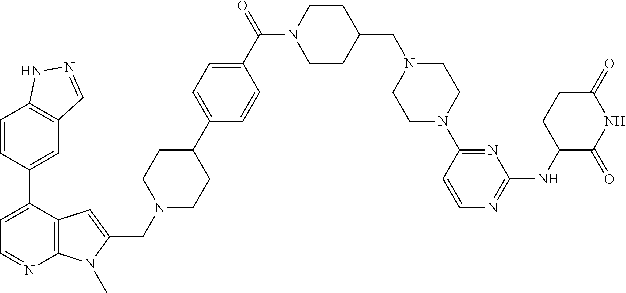 [SMILES: Cn1c(CN2CCC(c3ccc(C(=O)N4CCC(CN5CCN(c6ccnc(NC7CCC(=O)NC7=O)n6)CC5)CC4)cc3)CC2)cc2c(-c3ccc4[nH]ncc4c3)ccnc21]

### Example 2k3. 3-((4-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)pyrimidin-2-yl)amino)piperidine-2,6-dione

Example 2k3 (7 mg) prepared similarly to Example 2b1 from Ints. 3A1/2K6 (9 mg, HCl salt/7 mg, TFA salt).

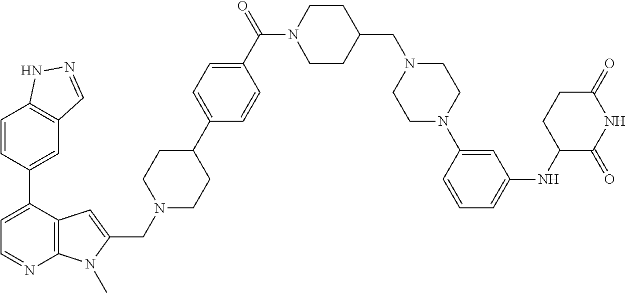 [SMILES: Cn1c(CN2CCC(c3ccc(C(=O)N4CCC(CN5CCN(c6cccc(NC7CCC(=O)NC7=O)c6)CC5)CC4)cc3)CC2)cc2c(-c3ccc4[nH]ncc4c3)ccnc21]

### Example 2l1. 3-((3-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)phenyl)amino)piperidine-2,6-dione

Example 2l1 (28 mg) prepared similarly to Example 2b1 from Ints. 3A1/2L1 (40 mg, HCl salt/38 mg, TFA salt).

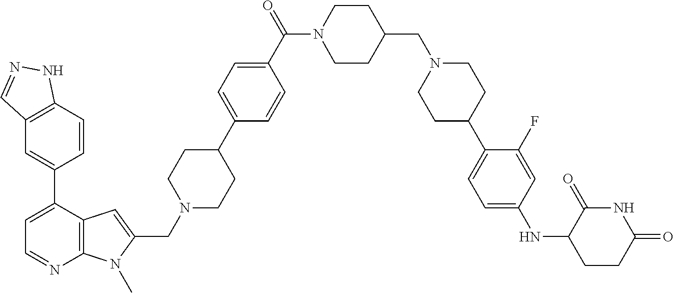 [SMILES: Cn1c(CN2CCC(c3ccc(C(=O)N4CCC(CN5CCC(c6ccc(NC7CCC(=O)NC7=O)cc6F)CC5)CC4)cc3)CC2)cc2c(-c3ccc4[nH]ncc4c3)ccnc21]

### Example 2m1. 3-((4-(1-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperidin-4-yl)-3-fluorophenyl)amino)piperidine-2,6-dione

Example 2m1 (21 mg) prepared similarly to Example 2b1 from Ints. 3A1/2M1 (25 mg, TFA salt/31 mg).

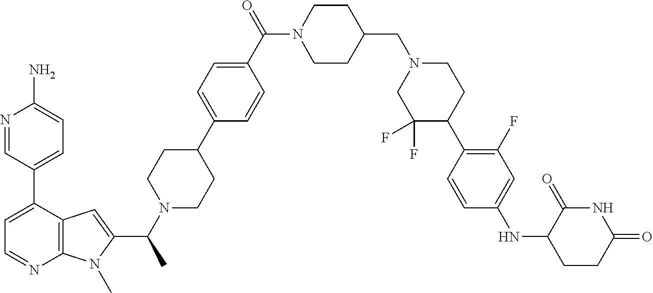 [SMILES: C[C@@H](c1cc2c(-c3ccc(N)nc3)ccnc2n1C)N1CCC(c2ccc(C(=O)N3CCC(CN4CCC(c5ccc(NC6CCC(=O)NC6=O)cc5F)C(F)(F)C4)CC3)cc2)CC1]

### Example 2m2. 3-((4-(1-((1-(4-(1-((S)-1-(4-(6-aminopyridin-3-yl)-1-methyl-1H-pyrrolo[2,3-b] pyridin-2-yl)-ethyl)piperidin-4-yl)benzoyl)piperidin-4-yl)methyl)-3,3-difluoropiperidin-4-yl)-3-fluorophenyl)amino)-piperidine-2,6-dione

Example 2m2 (40 mg) prepared similarly to Example 2c18 from Int. 2M6 (0.11 g).

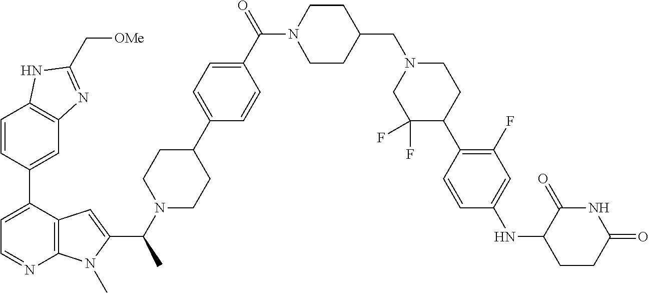 [SMILES: COCc1nc2cc(-c3ccnc4c3cc([C@H](C)N3CCC(c5ccc(C(=O)N6CCC(CN7CCC(c8ccc(NC9CCC(=O)NC9=O)cc8F)C(F)(F)C7)CC6)cc5)CC3)n4C)ccc2[nH]1]

### Example 2m3. 3-((4-(3,3-difluoro-1-((1-(4-(1-((S)-1-(4-(2-(methoxymethyl)-1H-benzo[d] imidazol-5-yl)-1-methyl-1H-pyrrolo[2,3-b]pyridin-2-yl)ethyl)piperidin-4-yl)benzoyl)piperidin-4-yl)methyl)piperidin-4-yl)-3-fluorophenyl)amino)piperidine-2,6-dione

Example 2m3 (17 mg) prepared similarly to Example 2c18 from Int. 2M13/2M14 (0.20 g).

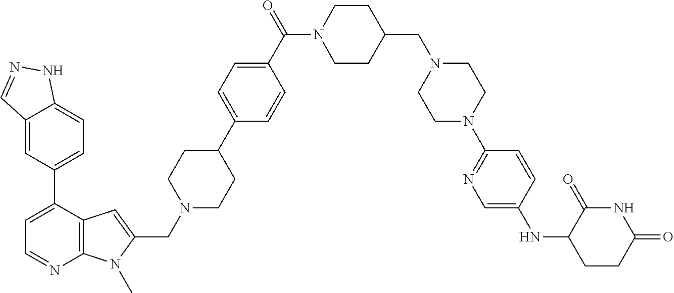 [SMILES: Cn1c(CN2CCC(c3ccc(C(=O)N4CCC(CN5CCN(c6ccc(NC7CCC(=O)NC7=O)cn6)CC5)CC4)cc3)CC2)cc2c(-c3ccc4[nH]ncc4c3)ccnc21]

### Example 2n1. 3-((6-(4-((1-(4-(1-((4-(1H-indazol-5-yl)-1-methyl-1H-pyrrolo[2,3-b]pyridin-2-yl)methyl)piperidin-4-yl)benzoyl)piperidin-4-yl)methyl)piperazin-1-yl)pyridin-3-yl)amino)piperidine-2,6-dione

Example 2n1 (17 mg) prepared similarly to Example 2b1 from Ints. 3A1/2N1 (28 mg, HCl salt/19 mg).

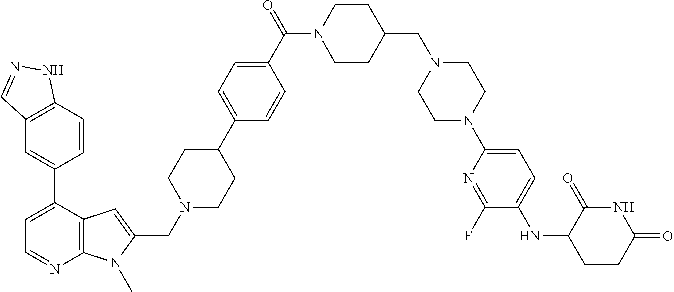 [SMILES: Cn1c(CN2CCC(c3ccc(C(=O)N4CCC(CN5CCN(c6ccc(NC7CCC(=O)NC7=O)c(F)n6)CC5)CC4)cc3)CC2)cc2c(-c3ccc4[nH]ncc4c3)ccnc21]

### Example 2n2. 3-((6-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)-2-fluoropyridin-3-yl)amino)piperidine-2,6-dione

Example 2n2 (15 mg) prepared similarly to Example 2b1 from Ints. 3A1/2N14 (35 mg, TFA salt/21 mg, HCl salt).

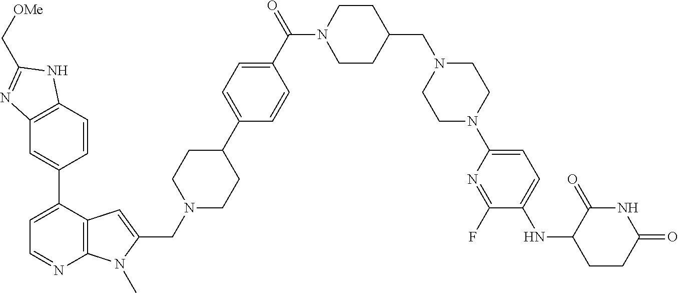 [SMILES: COCc1nc2cc(-c3ccnc4c3cc(CN3CCC(c5ccc(C(=O)N6CCC(CN7CCN(c8ccc(NC9CCC(=O)NC9=O)c(F)n8)CC7)CC6)cc5)CC3)n4C)ccc2[nH]1]

### Example 2n3. 3-((2-fluoro-6-(4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperazin-1-yl)pyridin-3-yl)amino)piperidine-2,6-dione

Example 2n3 (20 mg) prepared similarly to Example 2b1 from Ints. 3E261/2N14 (26 mg/26 mg, HCl salt).

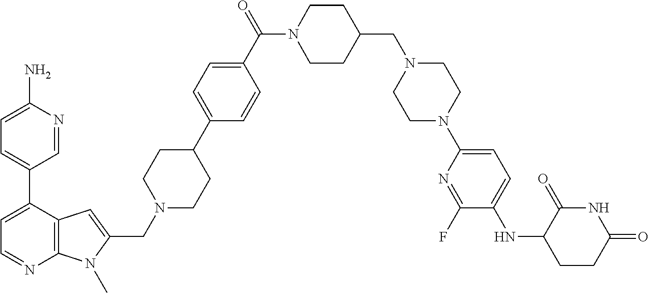 [SMILES: Cn1c(CN2CCC(c3ccc(C(=O)N4CCC(CN5CCN(c6ccc(NC7CCC(=O)NC7=O)c(F)n6)CC5)CC4)cc3)CC2)cc2c(-c3ccc(N)nc3)ccnc21]

### Example 2n4. 3-((6-(4-((1-(4-(1-((4-(6-aminopyridin-3-yl)-1-methyl-1H-pyrrolo[2,3-b]pyridin-2-yl)methyl)-piperidin-4-yl)benzoyl)piperidin-4-yl)methyl)piperazin-1-yl)-2-fluoropyridin-3-yl)amino)piperidine-2,6-dione

Example 2n4 (11 mg) prepared similarly to Example 2b1 from Ints. 3E23/2N14 (16 mg/18 mg, HCl salt).

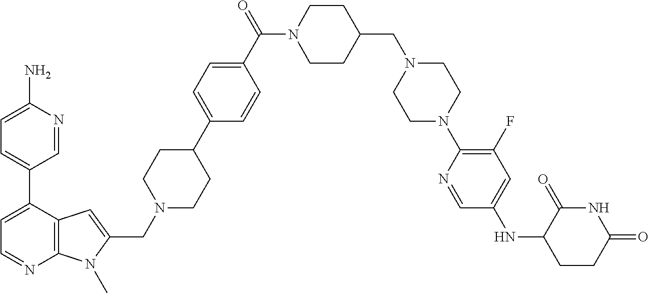 [SMILES: Cn1c(CN2CCC(c3ccc(C(=O)N4CCC(CN5CCN(c6ncc(NC7CCC(=O)NC7=O)cc6F)CC5)CC4)cc3)CC2)cc2c(-c3ccc(N)nc3)ccnc21]

### Example 2n5. 3-((6-(4-((1-(4-(1-((4-(6-aminopyridin-3-yl)-1-methyl-1H-pyrrolo[2,3-b]pyridin-2-yl)methyl)-piperidin-4-yl)benzoyl)piperidin-4-yl)methyl)piperazin-1-yl)-5-fluoropyridin-3-yl)amino)piperidine-2,6-dione

Example 2n5 (15 mg) prepared similarly to Example 2c18 from Int. 2N21 (20 mg).

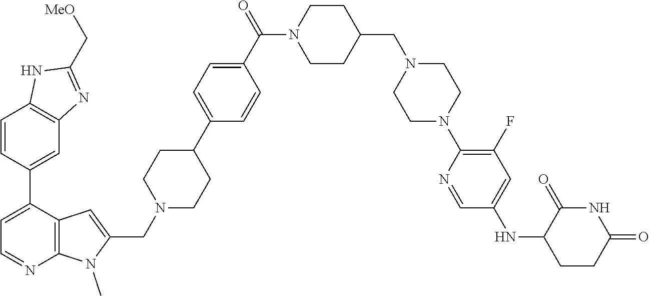 [SMILES: COCc1nc2cc(-c3ccnc4c3cc(CN3CCC(c5ccc(C(=O)N6CCC(CN7CCN(c8ncc(NC9CCC(=O)NC9=O)cc8F)CC7)CC6)cc5)CC3)n4C)ccc2[nH]1]

### Example 2n6. 3-((5-fluoro-6-(4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperazin-1-yl)pyridin-3-yl)amino)piperidine-2,6-dione

Example 2n6 (9 mg) prepared similarly to Example 2f3 from Int. 2N23/2N24 (15 mg).

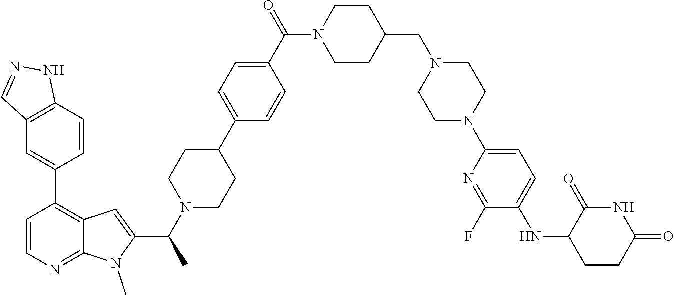 [SMILES: C[C@@H](c1cc2c(-c3ccc4[nH]ncc4c3)ccnc2n1C)N1CCC(c2ccc(C(=O)N3CCC(CN4CCN(c5ccc(NC6CCC(=O)NC6=O)c(F)n5)CC4)CC3)cc2)CC1]

### Example 2n7. 3-((6-(4-((1-(4-(1-((S)-1-(4-(1H-indazol-5-yl)-1-methyl-1H-pyrrolo[2,3-b]pyridin-2-yl)ethyl)-piperidin-4-yl)benzoyl)piperidin-4-yl)methyl)piperazin-1-yl)-2-fluoropyridin-3-yl)amino)piperidine-2,6-dione

Example 2n7 (56 mg) prepared similarly to Example 2f3 from Int. 2N25 (104 mg).

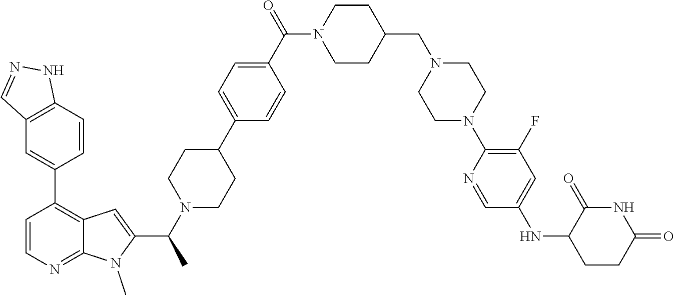 [SMILES: C[C@@H](c1cc2c(-c3ccc4[nH]ncc4c3)ccnc2n1C)N1CCC(c2ccc(C(=O)N3CCC(CN4CCN(c5ncc(NC6CCC(=O)NC6=O)cc5F)CC4)CC3)cc2)CC1]

### Example 2n8. 3-((6-(4-((1-(4-(1-((S)-1-(4-(1H-indazol-5-yl)-1-methyl-1H-pyrrolo[2,3-b]pyridin-2-yl)ethyl)-piperidin-4-yl)benzoyl)piperidin-4-yl)methyl)piperazin-1-yl)-5-fluoropyridin-3-yl)amino)piperidine-2,6-dione

Example 2n8 (38 mg) prepared similarly to Example 2f3 from Int. 2N26 (61 mg).

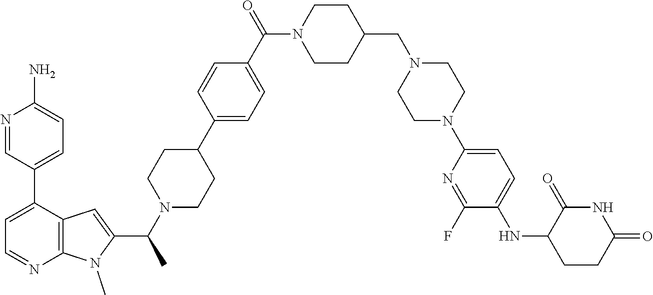 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)c(F)n5)CC4)CC3)cc2)CC1]

### Example 2n9. 3-((6-(4-((1-(4-(1-((S)-1-(4-(6-aminopyridin-3-yl)-1-methyl-1H-pyrrolo[2,3-b] pyridin-2-yl)-ethyl)-piperidin-4-yl)benzoyl)piperidin-4-yl)methyl)piperazin-1-yl)-2-fluoropyridin-3-yl)amino)piperidine-2,6-dione

Example 2n9 (68 mg) prepared similarly to Example 2f3 from Int. 2N27 (0.10 g).

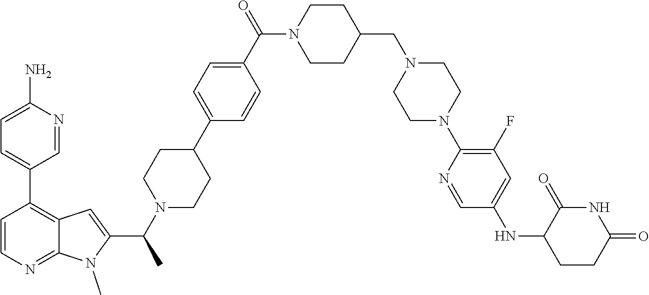 [SMILES: C[C@@H](c1cc2c(-c3ccc(N)nc3)ccnc2n1C)N1CCC(c2ccc(C(=O)N3CCC(CN4CCN(c5ncc(NC6CCC(=O)NC6=O)cc5F)CC4)CC3)cc2)CC1]

### Example 2n10. 3-((6-(4-((1-(4-(1-((S)-1-(4-(6-aminopyridin-3-yl)-1-methyl-1H-pyrrolo[2,3-b] pyridin-2-yl)ethyl)-piperidin-4-yl)benzoyl)piperidin-4-yl)methyl)piperazin-1-yl)-5-fluoropyridin-3-yl)amino)-piperidine-2,6-dione

Example 2n10 (65 mg) prepared similarly to Example 2b1 from HCl salts of Ints. 3E83/2N5 (0.30 g/0.35 g).

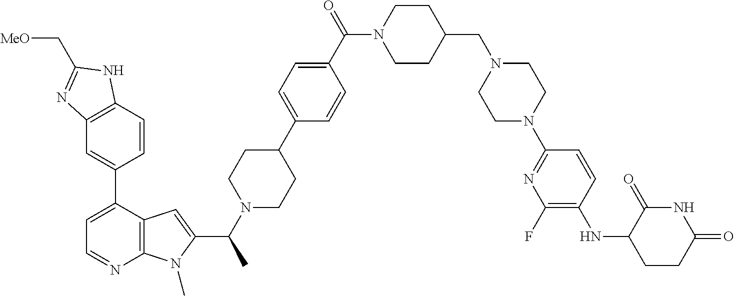 [SMILES: COCc1nc2cc(-c3ccnc4c3cc([C@H](C)N3CCC(c5ccc(C(=O)N6CCC(CN7CCN(c8ccc(NC9CCC(=O)NC9=O)c(F)n8)CC7)CC6)cc5)CC3)n4C)ccc2[nH]1]

### Example 2n11. 3-((2-fluoro-6-(4-((1-(4-(1-((S)-1-(4-(2-(methoxymethyl)-1H-benzo[d]-imidazol-5-yl)-1-methyl-1H-pyrrolo[2,3-b]pyridin-2-yl)ethyl)piperidin-4-yl)benzoyl)-piperidin-4-yl)methyl)piperazin-1-yl)pyridin-3-yl)amino)piperidine-2,6-dione

Example 2n11 (12 mg) prepared similarly to Example 2c18 from Int. 2N11/2N12 (70 mg).

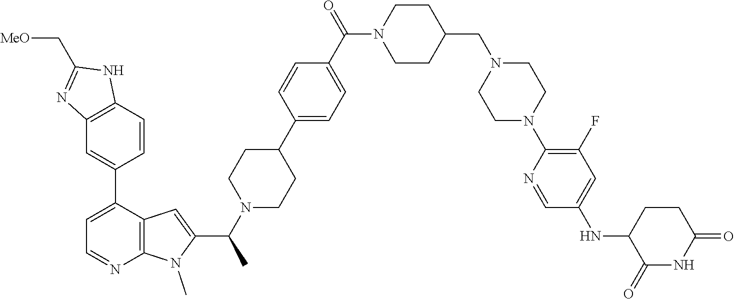 [SMILES: COCc1nc2cc(-c3ccnc4c3cc([C@H](C)N3CCC(c5ccc(C(=O)N6CCC(CN7CCN(c8ncc(NC9CCC(=O)NC9=O)cc8F)CC7)CC6)cc5)CC3)n4C)ccc2[nH]1]

### Example 2n12. 3-((5-fluoro-6-(4-((1-(4-(1-((S)-1-(4-(2-(methoxymethyl)-1H-benzo[d]imidazol-5-yl)-1-methyl-1H-pyrrolo[2,3-b]pyridin-2-yl)ethyl)piperidin-4-yl)benzoyl)piperidin-4-yl)methyl)piperazin-1-yl)pyridin-3-yl)amino)piperidine-2,6-dione

Example 2n12 (25 mg) prepared similarly to Example 2c18 from Int. 2N18/2N19 (0.10 g).

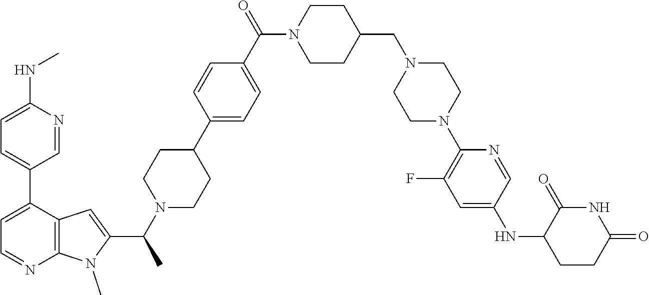 [SMILES: CNc1ccc(-c2ccnc3c2cc([C@H](C)N2CCC(c4ccc(C(=O)N5CCC(CN6CCN(c7ncc(NC8CCC(=O)NC8=O)cc7F)CC6)CC5)cc4)CC2)n3C)cn1]

### Example 2n13. 3-((5-fluoro-6-(4-((1-(4-(1-((S)-1-(1-methyl-4-(6-(methylamino)pyridin-3-yl)-1H-pyrrolo[2,3-b]pyridin-2-yl)ethyl)piperidin-4-yl)benzoyl)piperidin-4-yl)methyl)-piperazin-1-yl)pyridin-3-yl)amino)piperidine-2,6-dione

Example 2n13 (13 mg) prepared similarly to Example 2c18 from Int. 2N20 (0.15 g).

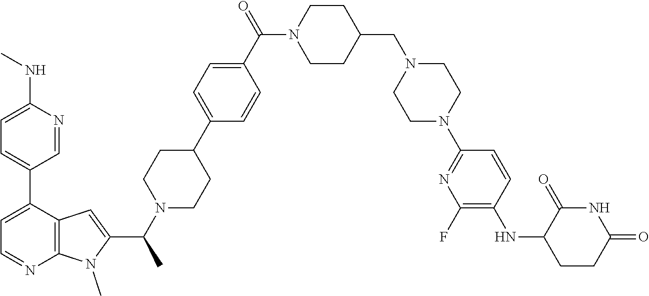 [SMILES: CNc1ccc(-c2ccnc3c2cc([C@H](C)N2CCC(c4ccc(C(=O)N5CCC(CN6CCN(c7ccc(NC8CCC(=O)NC8=O)c(F)n7)CC6)CC5)cc4)CC2)n3C)cn1]

### Example 2n14. 3-((2-fluoro-6-(4-((1-(4-(1-((S)-1-(1-methyl-4-(6-(methylamino)pyridin-3-yl)-1H-pyrrolo[2,3-b]pyridin-2-yl)ethyl)piperidin-4-yl)benzoyl)piperidin-4-yl)methyl)piperazin-1-yl)pyridin-3-yl)amino)piperidine-2,6-dione

Example 2n14 (63 mg) prepared similarly to Example 2c18 from Int. 2N13 (0.18 g).

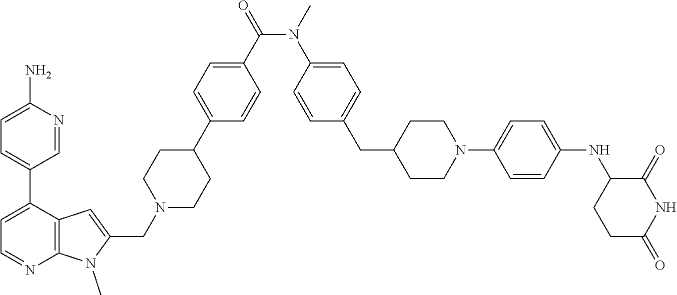 [SMILES: CN(C(=O)c1ccc(C2CCN(Cc3cc4c(-c5ccc(N)nc5)ccnc4n3C)CC2)cc1)c1ccc(CC2CCN(c3ccc(NC4CCC(=O)NC4=O)cc3)CC2)cc1]

### Example 2o1. 4-(1-((4-(6-aminopyridin-3-yl)-1-methyl-1H-pyrrolo[2,3-b]pyridin-2-yl)-methyl)piperidin-4-yl)-N-(4-((1-(4-((2,6-dioxopiperidin-3-yl)amino)phenyl) piperidin-4-yl)methyl) phenyl)-N-methylbenzamide

Int. 2o1 (50 mg), 5-(4,4,5,5-tetramethyl-1,3,2-dioxaborolan-2-yl)pyridin-2-amine (13 mg), and PdDPPFCl₂-DCM (4 mg) were degassed in DMF/10% aq. Na₂CO₃ (15:1, 1.6 mL) and stirred 1.5 h at 100° C. Filtration, concentration, and HPLC-purification gave Example 2o1 (9 mg).

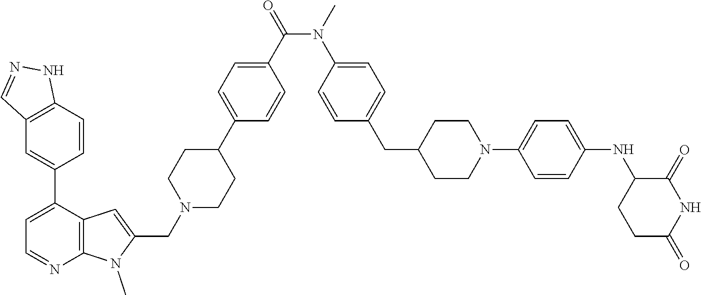 [SMILES: CN(C(=O)c1ccc(C2CCN(Cc3cc4c(-c5ccc6[nH]ncc6c5)ccnc4n3C)CC2)cc1)c1ccc(CC2CCN(c3ccc(NC4CCC(=O)NC4=O)cc3)CC2)cc1]

### Example 2o2. 4-(1-((4-(1H-indazol-5-yl)-1-methyl-1H-pyrrolo[2,3-b]pyridin-2-yl)methyl)-piperidin-4-yl)-N-(4-((1-(4-((2,6-dioxopiperidin-3-yl)amino)phenyl) piperidin-4-yl)methyl)-phenyl)-N-methylbenzamide

Example 2o2 (7 mg) prepared similarly to Example 2f3 from Int. 2O6 (46 mg).

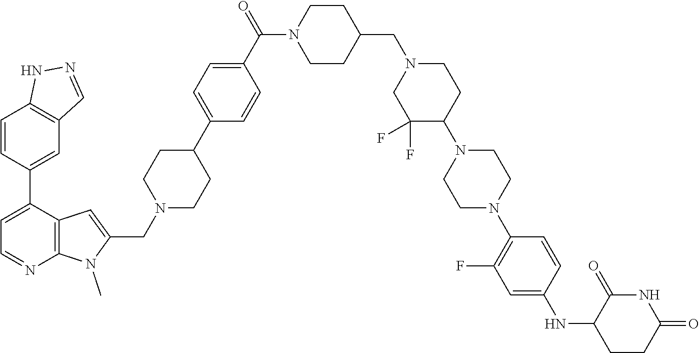 [SMILES: Cn1c(CN2CCC(c3ccc(C(=O)N4CCC(CN5CCC(N6CCN(c7ccc(NC8CCC(=O)NC8=O)cc7F)CC6)C(F)(F)C5)CC4)cc3)CC2)cc2c(-c3ccc4[nH]ncc4c3)ccnc21]

### Example 2p1. 3-((4-(4-(1-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)-3,3-difluoropiperidin-4-yl) piperazin-1-yl)-3-fluorophenyl)-amino)piperidine-2,6-dione

Example 2p1 (15 mg) prepared similarly to Example 2f3 from Int. 2P1 (25 mg).

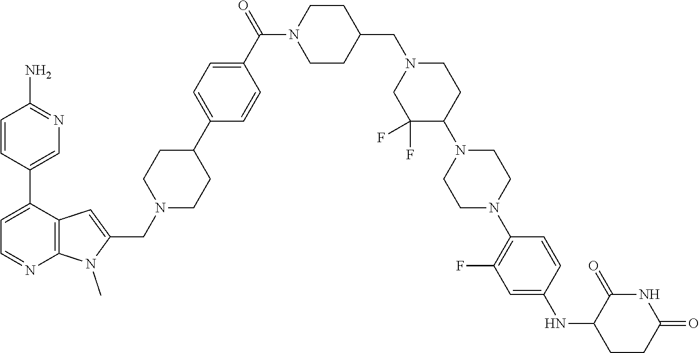 [SMILES: Cn1c(CN2CCC(c3ccc(C(=O)N4CCC(CN5CCC(N6CCN(c7ccc(NC8CCC(=O)NC8=O)cc7F)CC6)C(F)(F)C5)CC4)cc3)CC2)cc2c(-c3ccc(N)nc3)ccnc21]

### Example 2p2. 3-((4-(4-(1-((1-(4-(1-((4-(6-aminopyridin-3-yl)-1-methyl-1H-pyrrolo[2,3-b] pyridin-2-yl)-methyl)piperidin-4-yl)benzoyl)piperidin-4-yl)methyl)-3,3-difluoropiperidin-4-yl) piperazin-1-yl)-3-fluorophenyl)amino)piperidine-2,6-dione

Example 2p2 (17 mg) prepared similarly to Example 2f3 from Int. 2P9 (27 mg).

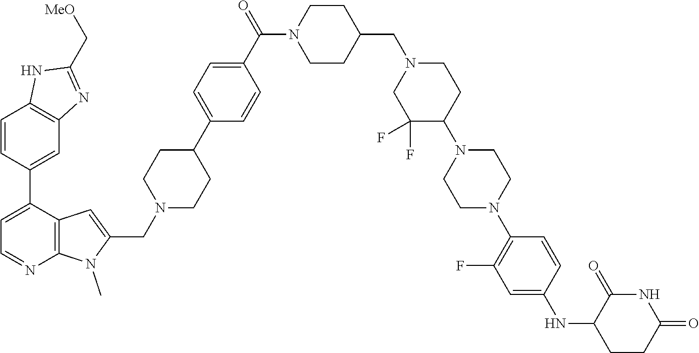 [SMILES: COCc1nc2cc(-c3ccnc4c3cc(CN3CCC(c5ccc(C(=O)N6CCC(CN7CCC(N8CCN(c9ccc(NC%10CCC(=O)NC%10=O)cc9F)CC8)C(F)(F)C7)CC6)cc5)CC3)n4C)ccc2[nH]1]

### Example 2p3. 3-((4-(4-(3,3-difluoro-1-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperidin-4-yl) piperazin-1-yl)-3-fluorophenyl)amino)piperidine-2,6-dione

Example 2p3 (12 mg) prepared similarly to Example 2f3 from Ints. 2P10/2P11 (20 mg).

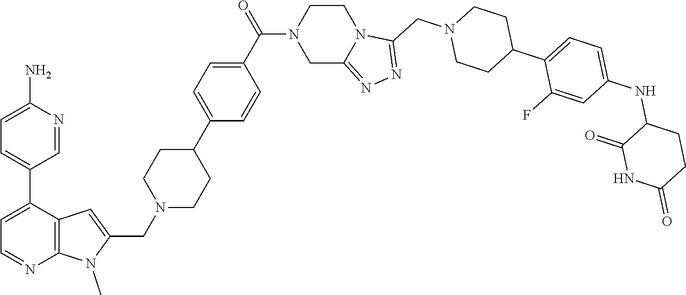 [SMILES: Cn1c(CN2CCC(c3ccc(C(=O)N4CCn5c(CN6CCC(c7ccc(NC8CCC(=O)NC8=O)cc7F)CC6)nnc5C4)cc3)CC2)cc2c(-c3ccc(N)nc3)ccnc21]

### Example 2q1. 3-((4-(1-((7-(4-(1-((4-(6-aminopyridin-3-yl)-1-methyl-1H-pyrrolo[2,3-b]pyridin-2-yl)methyl)piperidin-4-yl)benzoyl)-5,6,7,8-tetrahydro-[1,2,4]triazolo[4,3-a]pyrazin-3-yl)methyl)piperidin-4-yl)-3-fluorophenyl)amino)piperidine-2,6-dione

Example 2q1 (4 mg) prepared similarly to Example 2c18 from Int. 2Q1 (80 mg).

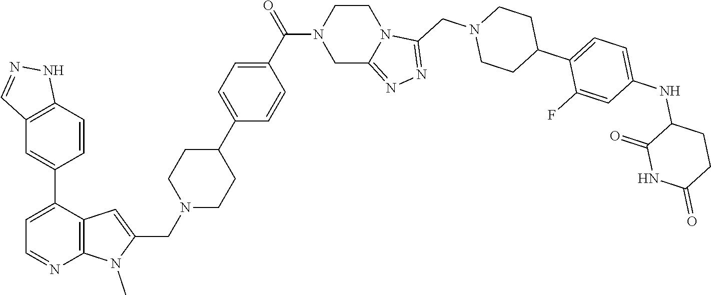 [SMILES: Cn1c(CN2CCC(c3ccc(C(=O)N4CCn5c(CN6CCC(c7ccc(NC8CCC(=O)NC8=O)cc7F)CC6)nnc5C4)cc3)CC2)cc2c(-c3ccc4[nH]ncc4c3)ccnc21]

### Example 2q2. 3-((4-(1-((7-(4-(1-((4-(1H-indazol-5-yl)-1-methyl-1H-pyrrolo[2,3-b]pyridin-2-yl)methyl)-piperidin-4-yl)benzoyl)-5,6,7,8-tetrahydro-[1,2,4]triazolo[4,3-a]pyrazin-3-yl)methyl)piperidin-4-yl)-3-fluorophenyl)amino)piperidine-2,6-dione

Example 2q2 (9 mg) prepared similarly to Example 2c18 from Int. 2Q9 (0.11 g).

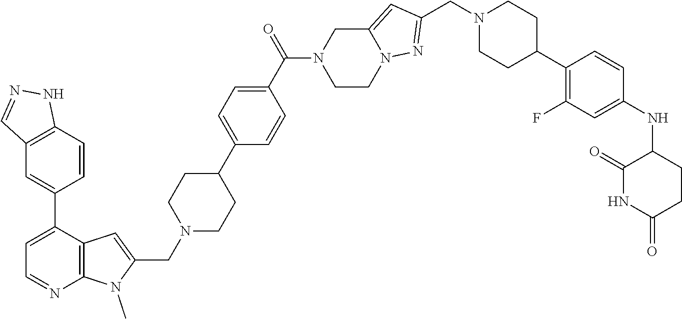 [SMILES: Cn1c(CN2CCC(c3ccc(C(=O)N4CCn5nc(CN6CCC(c7ccc(NC8CCC(=O)NC8=O)cc7F)CC6)cc5C4)cc3)CC2)cc2c(-c3ccc4[nH]ncc4c3)ccnc21]

### Example 2q3. 3-((4-(1-((5-(4-(1-((4-(1H-indazol-5-yl)-1-methyl-1H-pyrrolo[2,3-b]pyridin-2-yl)methyl)-piperidin-4-yl)benzoyl)-4,5,6,7-tetrahydropyrazolo[1,5-a]pyrazin-2-yl)methyl)piperidin-4-yl)-3-fluorophenyl)amino)piperidine-2,6-dione

Example 2q3 (19 mg) prepared similarly to Example 2c18 from Int. 2Q10 (60 mg).

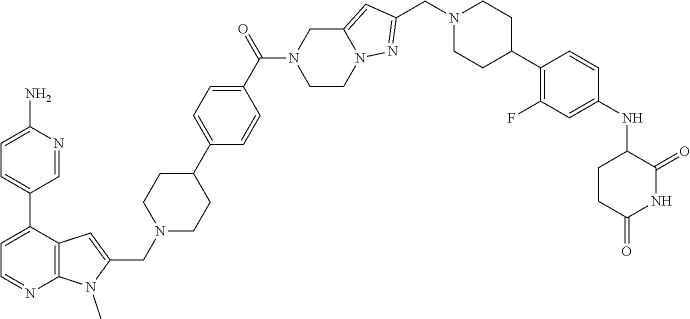 [SMILES: Cn1c(CN2CCC(c3ccc(C(=O)N4CCn5nc(CN6CCC(c7ccc(NC8CCC(=O)NC8=O)cc7F)CC6)cc5C4)cc3)CC2)cc2c(-c3ccc(N)nc3)ccnc21]

### Example 2q4. 3-((4-(1-((5-(4-(1-((4-(6-aminopyridin-3-yl)-1-methyl-1H-pyrrolo[2,3-b]pyridin-2-yl)methyl)-piperidin-4-yl)benzoyl)-4,5,6,7-tetrahydropyrazolo[1,5-a]pyrazin-2-yl)methyl)piperidin-4-yl)-3-fluoro-phenyl)amino)piperidine-2,6-dione

Example 2q4 (13 mg) prepared similarly to Example 2b1 from HCl salts of Ints. 3E23/2Q15 (0.15 g/0.22 g).

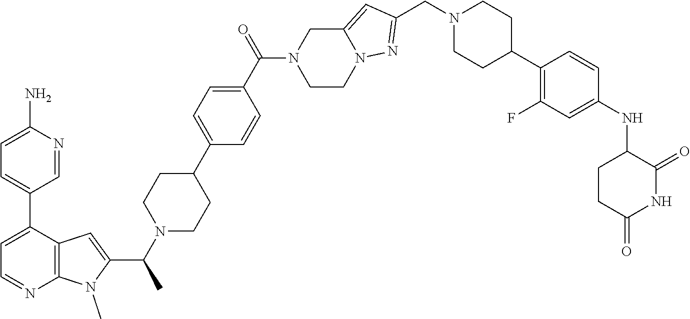 [SMILES: C[C@@H](c1cc2c(-c3ccc(N)nc3)ccnc2n1C)N1CCC(c2ccc(C(=O)N3CCn4nc(CN5CCC(c6ccc(NC7CCC(=O)NC7=O)cc6F)CC5)cc4C3)cc2)CC1]

### Example 2q5. 3-((4-(1-((5-(4-(1-((S)-1-(4-(6-aminopyridin-3-yl)-1-methyl-1H-pyrrolo[2,3-b] pyridin-2-yl)ethyl)-piperidin-4-yl)benzoyl)-4,5,6,7-tetrahydropyrazolo[1,5-a]pyrazin-2-yl)methyl)piperidin-4-yl)-3-fluorophenyl)-amino)piperidine-2,6-dione

Example 2q5 (0.5 g) prepared similarly to Example 2b1 from HCl salts of Ints. 3E83/2Q15 (1.0 g/0.8 g).

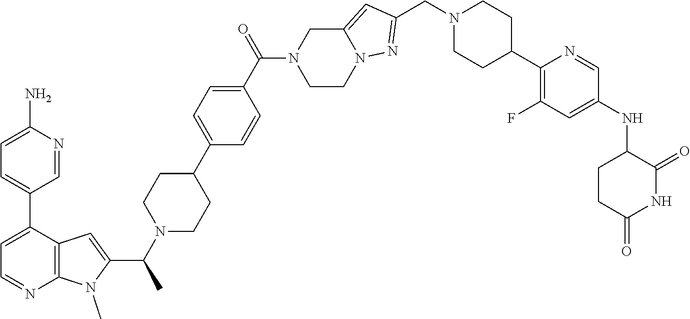 [SMILES: C[C@@H](c1cc2c(-c3ccc(N)nc3)ccnc2n1C)N1CCC(c2ccc(C(=O)N3CCn4nc(CN5CCC(c6ncc(NC7CCC(=O)NC7=O)cc6F)CC5)cc4C3)cc2)CC1]

### Example 2q6. 3-((6-(1-((5-(4-(1-((S)-1-(4-(6-aminopyridin-3-yl)-1-methyl-1H-pyrrolo[2,3-b] pyridin-2-yl)ethyl)-piperidin-4-yl)benzoyl)-4,5,6,7-tetrahydropyrazolo[1,5-a]pyrazin-2-yl)methyl)piperidin-4-yl)-5-fluoropyridin-3-yl)amino)piperidine-2,6-dione

Example 2q6 (41 mg) prepared similarly to Example 2b1 from HCl salts of Ints. 3E83/2Q16 (0.18 g/0.20 g).

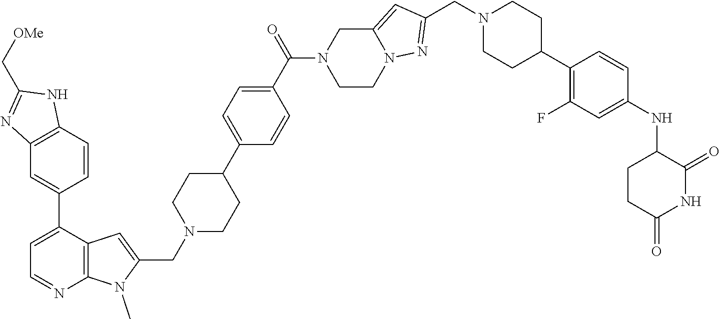 [SMILES: COCc1nc2cc(-c3ccnc4c3cc(CN3CCC(c5ccc(C(=O)N6CCn7nc(CN8CCC(c9ccc(NC%10CCC(=O)NC%10=O)cc9F)CC8)cc7C6)cc5)CC3)n4C)ccc2[nH]1]

### Example 2q7. 3-((3-fluoro-4-(1-((5-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)-4,5,6,7-tetrahydropyrazolo[1,5-a]pyrazin-2-yl)-methyl)piperidin-4-yl)phenyl)amino)piperidine-2,6-dione

Example 2q7 (35 mg) prepared similarly to Example 2b1 from HCl salts of Ints. 3E261/2Q15 (0.15 g/0.2 g).

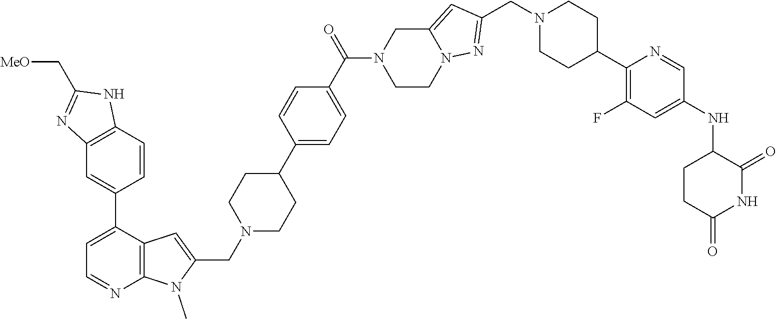 [SMILES: COCc1nc2cc(-c3ccnc4c3cc(CN3CCC(c5ccc(C(=O)N6CCn7nc(CN8CCC(c9ncc(NC%10CCC(=O)NC%10=O)cc9F)CC8)cc7C6)cc5)CC3)n4C)ccc2[nH]1]

### Example 2q8. 3-((5-fluoro-6-(1-((5-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)-4,5,6,7-tetrahydropyrazolo[1,5-a]pyrazin-2-yl)methyl)piperidin-4-yl)pyridin-3-yl)amino)piperidine-2,6-dione

Example 2q8 (25 mg) prepared similarly to Example 2b1 from Ints. 3E261/2Q16 (0.12 g/0.12 g, HCl salt).

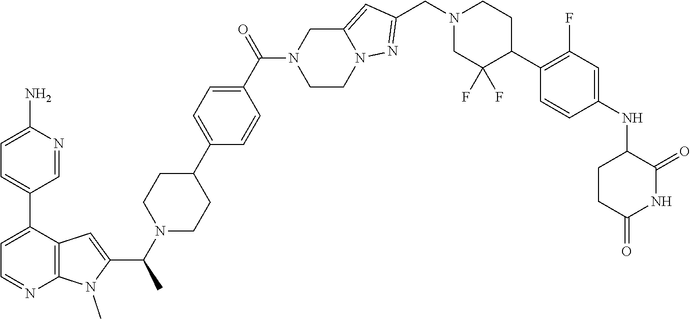 [SMILES: C[C@@H](c1cc2c(-c3ccc(N)nc3)ccnc2n1C)N1CCC(c2ccc(C(=O)N3CCn4nc(CN5CCC(c6ccc(NC7CCC(=O)NC7=O)cc6F)C(F)(F)C5)cc4C3)cc2)CC1]

### Example 2q9. 3-((4-(1-((5-(4-(1-((S)-1-(4-(6-aminopyridin-3-yl)-1-methyl-1H-pyrrolo[2,3-b] pyridin-2-yl)-ethyl)piperidin-4-yl)benzoyl)-4,5,6,7-tetrahydropyrazolo[1,5-a]pyrazin-2-yl)methyl)-3,3-difluoro-piperidin-4-yl)-3-fluorophenyl)amino)piperidine-2,6-dione

Example 2q9 (9 mg) prepared similarly to Example 2b1 from HCl salts of Ints. 3E83/2Q23 (80 mg/83 mg).

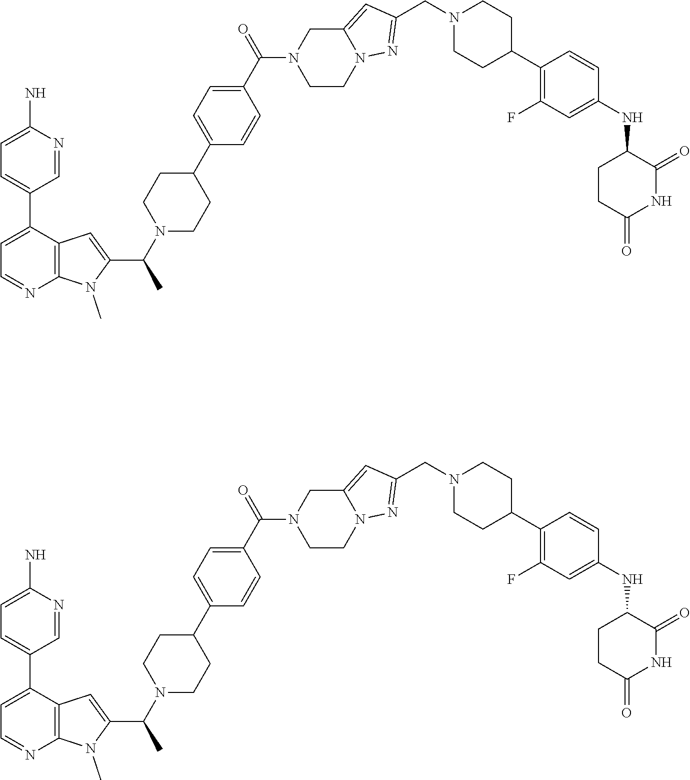 [SMILES: C[C@@H](c1cc2c(-c3ccc(N)nc3)ccnc2n1C)N1CCC(c2ccc(C(=O)N3CCn4nc(CN5CCC(c6ccc(N[C@@H]7CCC(=O)NC7=O)cc6F)CC5)cc4C3)cc2)CC1 ; C[C@@H](c1cc2c(-c3ccc(N)nc3)ccnc2n1C)N1CCC(c2ccc(C(=O)N3CCn4nc(CN5CCC(c6ccc(N[C@H]7CCC(=O)NC7=O)cc6F)CC5)cc4C3)cc2)CC1]

### Example 2q10 and Example 2q11. (R)-3-((4-(1-((5-(4-(1-((S)-1-(4-(6-aminopyridin-3-yl)-1-methyl-1H-pyrrolo[2,3-b]pyridin-2-yl)ethyl)piperidin-4-yl)benzoyl)-4,5,6,7-tetrahydropyrazolo[1,5-a]pyrazin-2-yl)methyl)piperidin-4-yl)-3-fluorophenyl)amino)piperidine-2,6-dione and(S)-3-((4-(1-((5-(4-(1-((S)-1-(4-(6-aminopyridin-3-yl)-1-methyl-1H-pyrrolo[2,3-b]pyridin-2-yl)ethyl)piperidin-4-yl)benzoyl)-4,5,6,7-tetrahydropyrazolo[1,5-a]pyrazin-2-yl)methyl)piperidin-4-yl)-3-fluorophenyl)amino)piperidine-2,6-dione

Example 2q5 (0.23 g) was separated by SFC using a Chiralpak IF 250×30 mm 5 μm column, an eluent of ACN: IPA: DCM (35:35:30), a flow rate of 42 mL/min. at RT to give Example 2q10 (46 mg, first eluting peak) and Example 2q11 (40 mg, second eluting peak). The configurations of the chiral centers in the glutarimide rings were not determined.

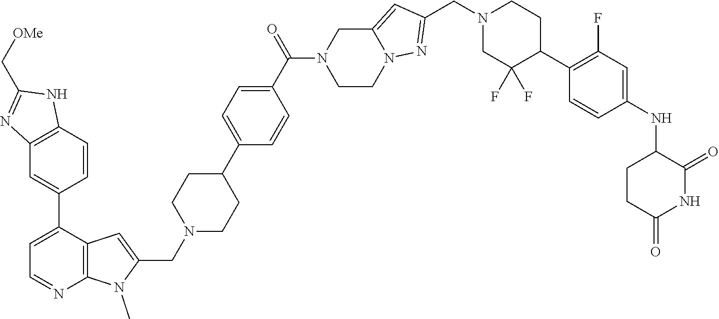 [SMILES: COCc1nc2cc(-c3ccnc4c3cc(CN3CCC(c5ccc(C(=O)N6CCn7nc(CN8CCC(c9ccc(NC%10CCC(=O)NC%10=O)cc9F)C(F)(F)C8)cc7C6)cc5)CC3)n4C)ccc2[nH]1]

### Example 2q12. 3-((4-(3,3-difluoro-1-((5-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)-4,5,6,7-tetrahydropyrazolo[1,5-a]pyrazin-2-yl)methyl)piperidin-4-yl)-3-fluorophenyl)amino)piperidine-2,6-dione

Example 2q12 (31 mg) prepared similarly to Example 2b1 from HCl salts of Ints. 3E261/2Q23 (0.20 g/0.34 g).

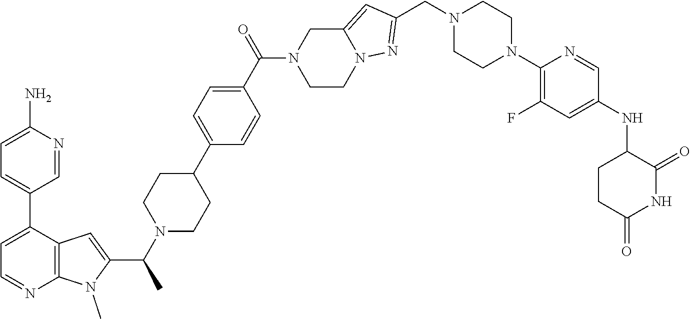 [SMILES: C[C@@H](c1cc2c(-c3ccc(N)nc3)ccnc2n1C)N1CCC(c2ccc(C(=O)N3CCn4nc(CN5CCN(c6ncc(NC7CCC(=O)NC7=O)cc6F)CC5)cc4C3)cc2)CC1]

### Example 2q14. 3-((6-(4-((5-(4-(1-((S)-1-(4-(6-aminopyridin-3-yl)-1-methyl-1H-pyrrolo[2,3-b] pyridin-2-yl)ethyl)-piperidin-4-yl)benzoyl)-4,5,6,7-tetrahydropyrazolo[1,5-a]pyrazin-2-yl)methyl)piperazin-1-yl)-5-fluoropyridin-3-yl)amino)piperidine-2,6-dione

Example 2q14 (28 mg) prepared similarly to Example 2b1 from Ints. 3E83/2Q27 (0.19 g/0.20 g).

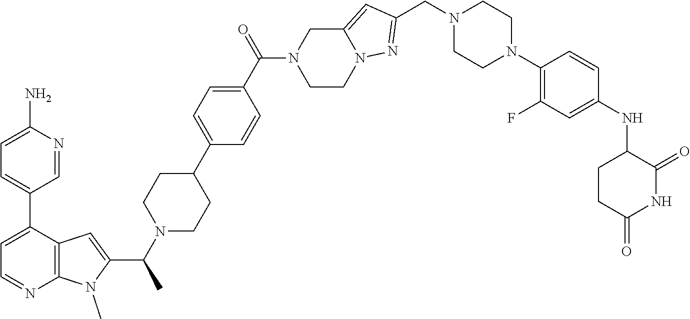 [SMILES: C[C@@H](c1cc2c(-c3ccc(N)nc3)ccnc2n1C)N1CCC(c2ccc(C(=O)N3CCn4nc(CN5CCN(c6ccc(NC7CCC(=O)NC7=O)cc6F)CC5)cc4C3)cc2)CC1]

### Example 2q15. 3-((4-(4-((5-(4-(1-((S)-1-(4-(6-aminopyridin-3-yl)-1-methyl-1H-pyrrolo[2,3-b] pyridin-2-yl)-ethyl)piperidin-4-yl)benzoyl)-4,5,6,7-tetrahydropyrazolo[1,5-a]pyrazin-2-yl)methyl)piperazin-1-yl)-3-fluoro-phenyl)amino)piperidine-2,6-dione

Example 2q15 (28 mg) prepared similarly to Example 2b1 from Ints. 3E83/2Q33 (0.19 g/0.20 g, HCl salt).

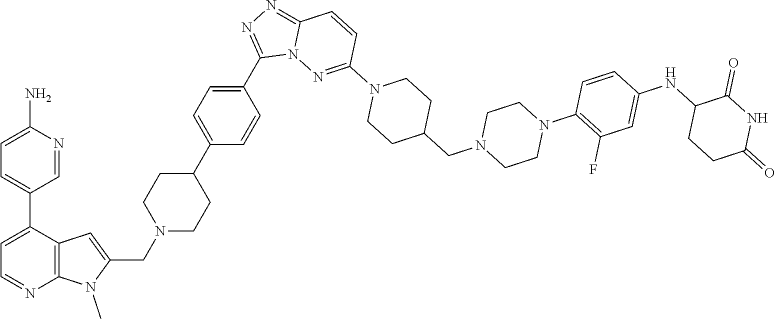 [SMILES: Cn1c(CN2CCC(c3ccc(-c4nnc5ccc(N6CCC(CN7CCN(c8ccc(NC9CCC(=O)NC9=O)cc8F)CC7)CC6)nn45)cc3)CC2)cc2c(-c3ccc(N)nc3)ccnc21]

### Example 2r1. 3-((4-(4-((1-(3-(4-(1-((4-(6-aminopyridin-3-yl)-1-methyl-1H-pyrrolo[2,3-b] pyridin-2-yl)methyl)-piperidin-4-yl)phenyl)-[1,2,4]triazolo[4,3-b]pyridazin-6-yl) piperidin-4-yl)methyl)piperazin-1-yl)-3-fluorophenyl)-amino)piperidine-2,6-dione

A solution of Ints. 3E297/2C15 (17 mg/14 mg) in DIPEA (0.02 mL) and NMP (1 mL) was stirred at 70° C. ON and 5 h at 100° C. The mixture was diluted with 2:1 ACN/water, filtered, and HPLC-purified, and purified by FC (DCM/MeOH/Et₃N 94.5:5:0.5) to give Int. 2r1 (2.2 mM in DMSO (0.20 mL)).

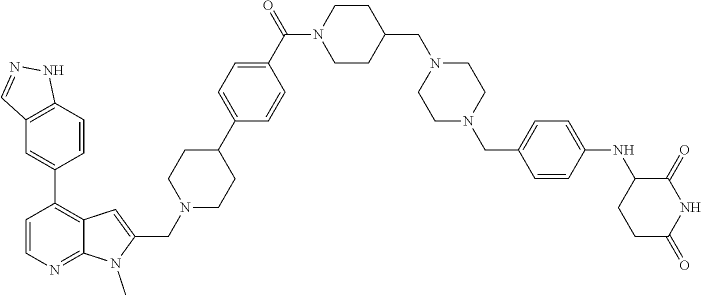 [SMILES: Cn1c(CN2CCC(c3ccc(C(=O)N4CCC(CN5CCN(Cc6ccc(NC7CCC(=O)NC7=O)cc6)CC5)CC4)cc3)CC2)cc2c(-c3ccc4[nH]ncc4c3)ccnc21]

### Example 2s1. 3-((4-((4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)methyl) phenyl)amino)piperidine-2,6-dione

Example 2s1 (3 mg) prepared similarly to Example 2g1 from Ints. 2G1/2S1 (37 mg/32 mg, TFA salt).

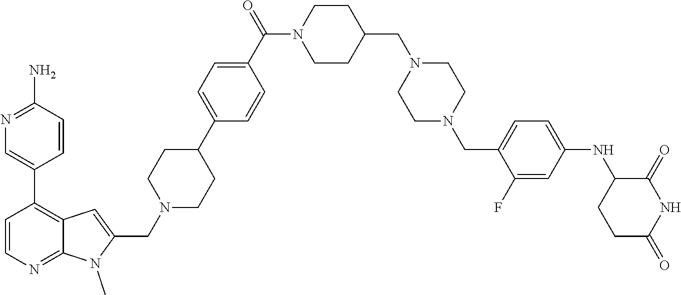 [SMILES: Cn1c(CN2CCC(c3ccc(C(=O)N4CCC(CN5CCN(Cc6ccc(NC7CCC(=O)NC7=O)cc6F)CC5)CC4)cc3)CC2)cc2c(-c3ccc(N)nc3)ccnc21]

### Example 2s2. 3-((4-((4-((1-(4-(1-((4-(6-aminopyridin-3-yl)-1-methyl-1H-pyrrolo[2,3-b]pyridin-2-yl)methyl)piperidin-4-yl)benzoyl)piperidin-4-yl)methyl)piperazin-1-yl)methyl)-3-fluorophenyl)amino)piperidine-2,6-dione

Int. 3A1 (34 mg, TFA salt) and HATU (31 mg) were stirred 0.5 h in DMF/DIPEA (53:1, 2 mL). Int 2S6 (33 mg, TFA salt) was added and stirring continued 2 h. The mixture was concentrated. The OL (2MeTHF/water) was washed with brine, dried, and HPLC-purified to give Example 2s2 (11 mg).

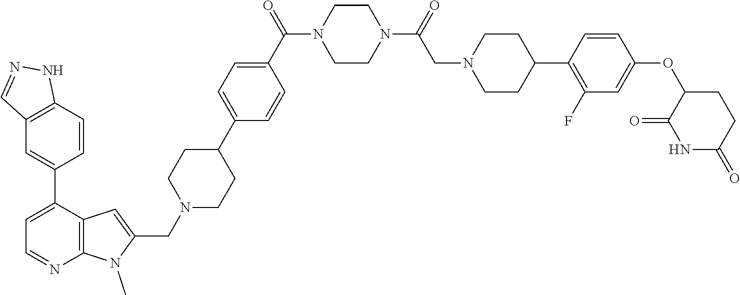 [SMILES: Cn1c(CN2CCC(c3ccc(C(=O)N4CCN(C(=O)CN5CCC(c6ccc(OC7CCC(=O)NC7=O)cc6F)CC5)CC4)cc3)CC2)cc2c(-c3ccc4[nH]ncc4c3)ccnc21]

### Example 2t1. 3-(4-(1-(2-(4-(4-(1-((4-(1H-indazol-5-yl)-1-methyl-1H-pyrrolo[2,3-b]pyridin-2-yl)methyl)-piperidin-4-yl)benzoyl) piperazin-1-yl)-2-oxoethyl)piperidin-4-yl)-3-fluorophenoxy) piperidine-2,6-dione

Example 2t1 (7 mg) prepared similarly to Example 2b1 from TFA salts of Ints. 3A1/2T1 (21 mg/15 mg).

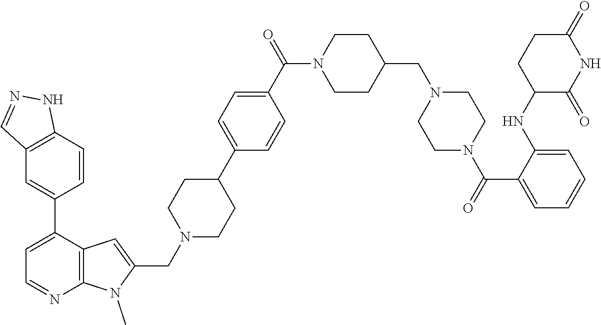 [SMILES: Cn1c(CN2CCC(c3ccc(C(=O)N4CCC(CN5CCN(C(=O)c6ccccc6NC6CCC(=O)NC6=O)CC5)CC4)cc3)CC2)cc2c(-c3ccc4[nH]ncc4c3)ccnc21]

### Example 2t2. 3-((2-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazine-1-carbonyl)phenyl)amino)piperidine-2,6-dione

Example 2t2 (18 mg) prepared similarly to Example 2g1 from TFA salts of Ints. 2G1/2T5 (25 mg/16 mg).

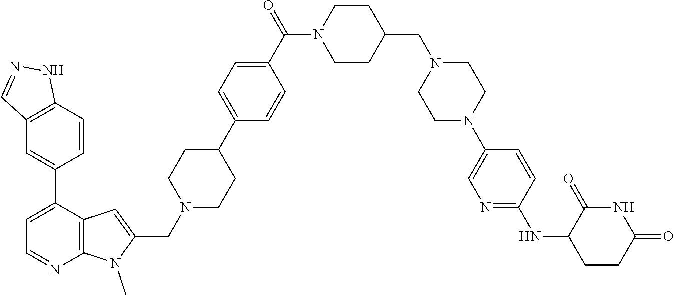 [SMILES: Cn1c(CN2CCC(c3ccc(C(=O)N4CCC(CN5CCN(c6ccc(NC7CCC(=O)NC7=O)nc6)CC5)CC4)cc3)CC2)cc2c(-c3ccc4[nH]ncc4c3)ccnc21]

### Example 2t3. 3-((5-(4-((1-(4-(1-((4-(1H-indazol-5-yl)-1-methyl-1H-pyrrolo[2,3-b]pyridin-2-yl)methyl)piperidin-4-yl)benzoyl)piperidin-4-yl)methyl)piperazin-1-yl)pyridin-2-yl)amino)piperidine-2,6-dione

Example 2t3 (17 mg) was prepared similarly Example 2a1 from Ints. 3A1/2T14 (27 mg/10 mg, TFA salt).

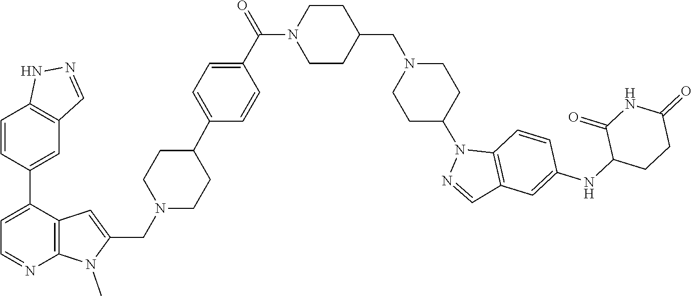 [SMILES: Cn1c(CN2CCC(c3ccc(C(=O)N4CCC(CN5CCC(n6ncc7cc(NC8CCC(=O)NC8=O)ccc76)CC5)CC4)cc3)CC2)cc2c(-c3ccc4[nH]ncc4c3)ccnc21]

### Example 2t4. 3-((1-(1-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperidin-4-yl)-1H-indazol-5-yl)amino)piperidine-2,6-dione

Example 2t4 (3 mg) prepared similarly to Example 2g1 from TFA salts of Ints. 2G1/2T8 (25 mg/25 mg).

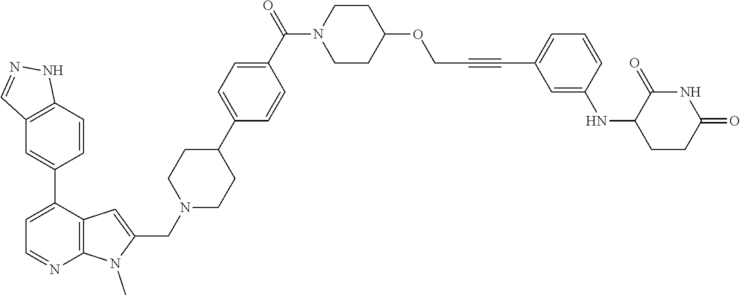 [SMILES: Cn1c(CN2CCC(c3ccc(C(=O)N4CCC(OCC#Cc5cccc(NC6CCC(=O)NC6=O)c5)CC4)cc3)CC2)cc2c(-c3ccc4[nH]ncc4c3)ccnc21]

### Example 2t5. 3-((3-(3-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)oxy) prop-1-yn-1-yl)phenyl)amino)-piperidine-2,6-dione

Example 2t5 (18 mg) prepared similarly to Example 2b1 from TFA salts of Ints. 3A1/2T12 (30 mg/25 mg).

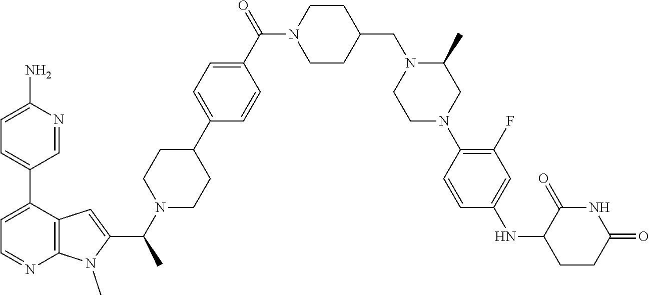 [SMILES: C[C@H]1CN(c2ccc(NC3CCC(=O)NC3=O)cc2F)CCN1CC1CCN(C(=O)c2ccc(C3CCN([C@@H](C)c4cc5c(-c6ccc(N)nc6)ccnc5n4C)CC3)cc2)CC1]

### Example 2c54. 3-((4-((S)-4-((1-(4-(1-((S)-1-(4-(6-aminopyridin-3-yl)-1-methyl-1H-pyrrolo[2,3-b] pyridin-2-yl)ethyl)piperidin-4-yl)benzoyl)piperidin-4-yl)methyl)-3-methylpiperazin-1-yl)-3-fluorophenyl)amino)piperidine-2,6-dione

Ints. 3E83/2F21 (0.10 g/0.14 g) and T3P (50% in EtOAc, 0.17 mL) were stirred 16 h in DMF/Et₃N (20:1, 3.2 mL). The mixture was diluted with water to precipitate a solid that was washed with Et₂O and HPLC-purified to give Example 2c54 (30 mg).

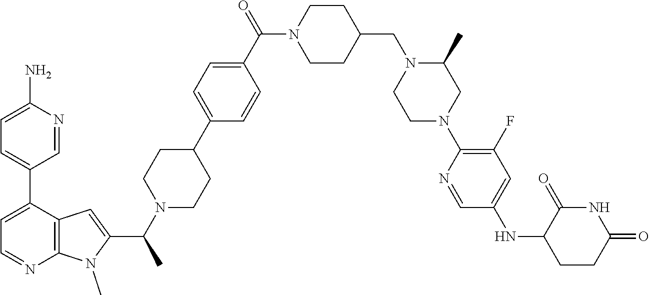 [SMILES: C[C@H]1CN(c2ncc(NC3CCC(=O)NC3=O)cc2F)CCN1CC1CCN(C(=O)c2ccc(C3CCN([C@@H](C)c4cc5c(-c6ccc(N)nc6)ccnc5n4C)CC3)cc2)CC1]

### Example 2c55. 3-((6-((S)-4-((1-(4-(1-((S)-1-(4-(6-aminopyridin-3-yl)-1-methyl-1H-pyrrolo[2,3-b] pyridin-2-yl)ethyl)piperidin-4-yl)benzoyl)piperidin-4-yl)methyl)-3-methylpiperazin-1-yl)-5-fluoropyridin-3-yl)amino)piperidine-2,6-dione

Example 2c55 (25 mg) was prepared similarly to Example 2c35 from Ints. 3E83/3E300 (0.10 g/0.16 g).

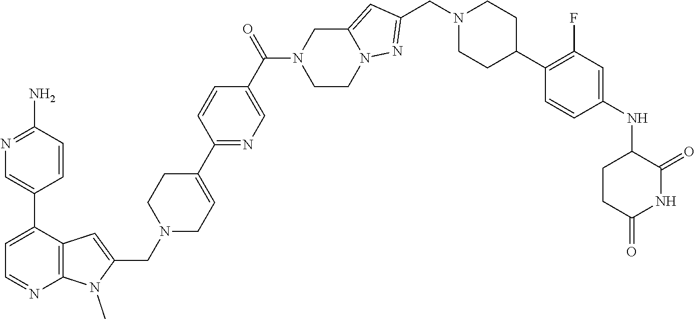 [SMILES: Cn1c(CN2CC=C(c3ccc(C(=O)N4CCn5nc(CN6CCC(c7ccc(NC8CCC(=O)NC8=O)cc7F)CC6)cc5C4)cn3)CC2)cc2c(-c3ccc(N)nc3)ccnc21]

### Example 2c37. 3-((4-(1-((5-(1′-((4-(6-aminopyridin-3-yl)-1-methyl-1H-pyrrolo[2,3-b]pyridin-2-yl)methyl)-1′, 2′, 3′, 6′-tetrahydro-[2,4′-bipyridine]-5-carbonyl)-4,5,6,7-tetrahydropyrazolo[1,5-a]pyrazin-2-yl)methyl)piperidin-4-yl)-3-fluorophenyl)amino)piperidine-2,6-dione

Int. 2C70 (34 mg) was stirred ON in DCM/TFA (2:1, 3 mL) and HPLC-purified to give Example 2c37 (3 mg).

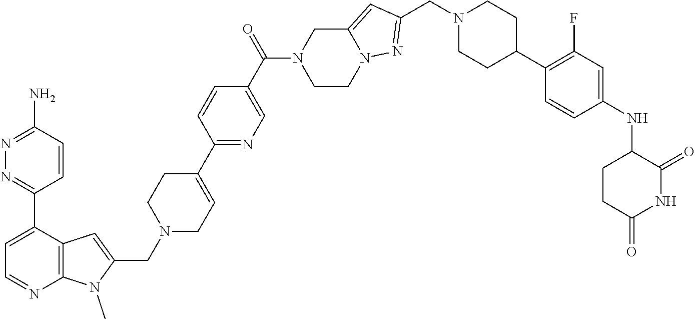 [SMILES: Cn1c(CN2CC=C(c3ccc(C(=O)N4CCn5nc(CN6CCC(c7ccc(NC8CCC(=O)NC8=O)cc7F)CC6)cc5C4)cn3)CC2)cc2c(-c3ccc(N)nn3)ccnc21]

### Example 2c38. 3-((4-(1-((5-(1′-((4-(6-aminopyridazin-3-yl)-1-methyl-1H-pyrrolo[2,3-b]pyridin-2-yl)methyl)-1′, 2′, 3′, 6′-tetrahydro-[2,4′-bipyridine]-5-carbonyl)-4,5,6,7-tetrahydropyrazolo[1,5-a]pyrazin-2-yl)methyl)piperidin-4-yl)-3-fluorophenyl)amino)piperidine-2,6-dione

Example 2c38 (5 mg) was prepared similarly to Example 2c37 from Int. 2C71 (34 mg).

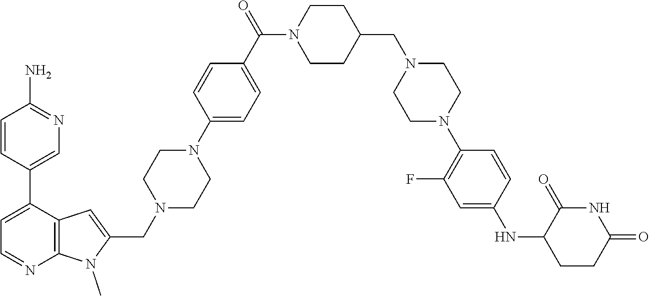 [SMILES: Cn1c(CN2CCN(c3ccc(C(=O)N4CCC(CN5CCN(c6ccc(NC7CCC(=O)NC7=O)cc6F)CC5)CC4)cc3)CC2)cc2c(-c3ccc(N)nc3)ccnc21]

### Example 2c39. 3-((4-(4-((1-(4-(4-((4-(6-aminopyridin-3-yl)-1-methyl-1H-pyrrolo[2,3-b]pyridin-2-yl)methyl)piperazin-1-yl)benzoyl)piperidin-4-yl)methyl)piperazin-1-yl)-3-fluorophenyl)amino)piperidine-2,6-dione

Example 2c39 (32 mg) was prepared similarly to Example 2c35 from Ints. 3C57/2C15 (0.20 g/0.20 g).

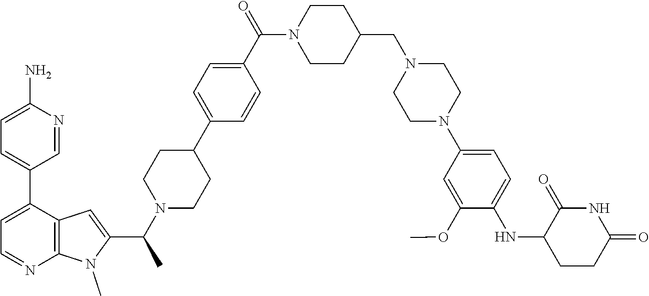 [SMILES: COc1cc(N2CCN(CC3CCN(C(=O)c4ccc(C5CCN([C@@H](C)c6cc7c(-c8ccc(N)nc8)ccnc7n6C)CC5)cc4)CC3)CC2)ccc1NC1CCC(=O)NC1=O]

### Example 2c40. 3-((4-(4-((1-(4-(1-((S)-1-(4-(6-aminopyridin-3-yl)-1-methyl-1H-pyrrolo[2,3-b] pyridin-2-yl)ethyl)piperidin-4-yl)benzoyl)piperidin-4-yl)methyl)piperazin-1-yl)-2-methoxyphenyl)amino)piperidine-2,6-dione

Example 2c40 (30 mg) was prepared similarly to Example 2c35 from Ints. 3E83/2C79 (0.10 g/0.15 g).

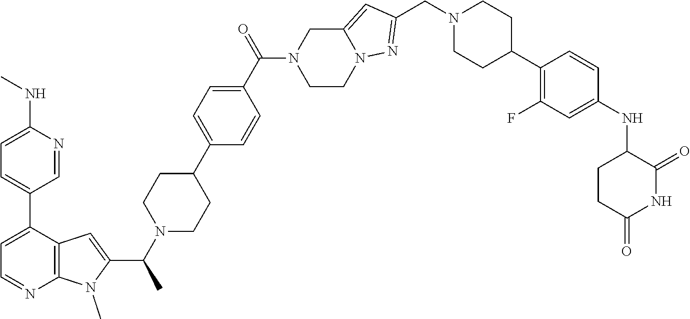 [SMILES: CNc1ccc(-c2ccnc3c2cc([C@H](C)N2CCC(c4ccc(C(=O)N5CCn6nc(CN7CCC(c8ccc(NC9CCC(=O)NC9=O)cc8F)CC7)cc6C5)cc4)CC2)n3C)cn1]

### Example 2c41. 3-((3-fluoro-4-(1-((5-(4-(1-((S)-1-(1-methyl-4-(6-(methylamino)pyridin-3-yl)-1H-pyrrolo[2,3-b]pyridin-2-yl)ethyl)piperidin-4-yl)benzoyl)-4,5,6,7-tetrahydropyrazolo[1,5-a]pyrazin-2-yl)methyl)piperidin-4-yl)phenyl)amino)piperidine-2,6-dione

Example 2c41 (0.15 g) was prepared similarly to Example 2c35 from Ints. 3C36/2Q15 (0.30 g/0.40 g).

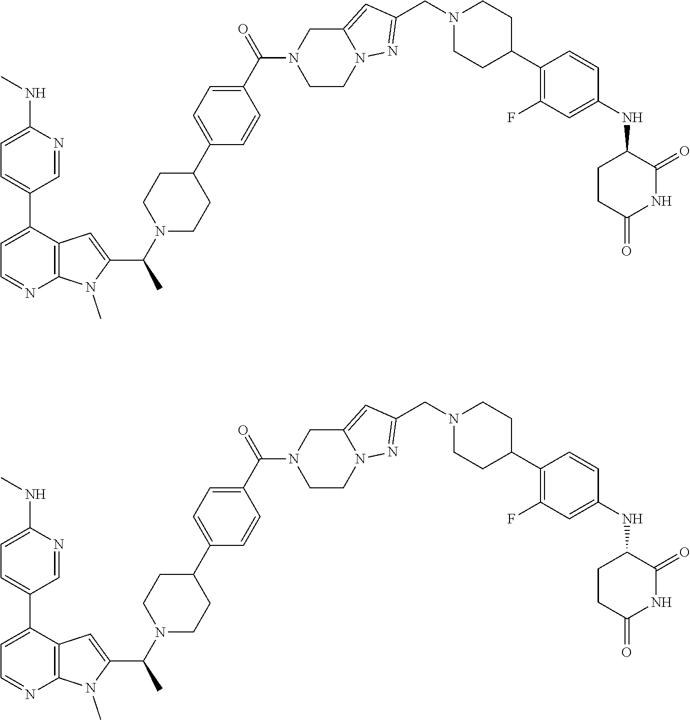 [SMILES: CNc1ccc(-c2ccnc3c2cc([C@H](C)N2CCC(c4ccc(C(=O)N5CCn6nc(CN7CCC(c8ccc(N[C@@H]9CCC(=O)NC9=O)cc8F)CC7)cc6C5)cc4)CC2)n3C)cn1 ; CNc1ccc(-c2ccnc3c2cc([C@H](C)N2CCC(c4ccc(C(=O)N5CCn6nc(CN7CCC(c8ccc(N[C@H]9CCC(=O)NC9=O)cc8F)CC7)cc6C5)cc4)CC2)n3C)cn1]

### Examples 2c42 and 2c43. (R)-3-((3-fluoro-4-(1-((5-(4-(1-((S)-1-(1-methyl-4-(6-(methylamino)pyridin-3-yl)-1H-pyrrolo[2,3-b]pyridin-2-yl)ethyl)piperidin-4-yl)benzoyl)-4,5,6,7-tetrahydropyrazolo[1,5-a]pyrazin-2-yl)methyl)piperidin-4-yl)phenyl)amino)piperidine-2,6-dione and(S)-3-((3-fluoro-4-(1-((5-(4-(1-((S)-1-(1-methyl-4-(6-(methylamino)pyridin-3-yl)-1H-pyrrolo[2,3-b]pyridin-2-yl)ethyl)piperidin-4-yl)benzoyl)-4,5,6,7-tetrahydropyrazolo[1,5-a]pyrazin-2-yl)methyl)piperidin-4-yl)phenyl)amino)piperidine-2,6-dione

Example 2c41 (0.10 g) was resolved by HPLC using a Chiralpak IK 250×10 mm 5 μm column with an eluent of 40% ACN, 40% IPA and 20% DCM (4 mL/min) to give Example 2c42 (24 mg, first peak) and Example 2c43 (35 mg, second peak). The absolute configurations of these compounds at the glutarimide chiral centers were not determined

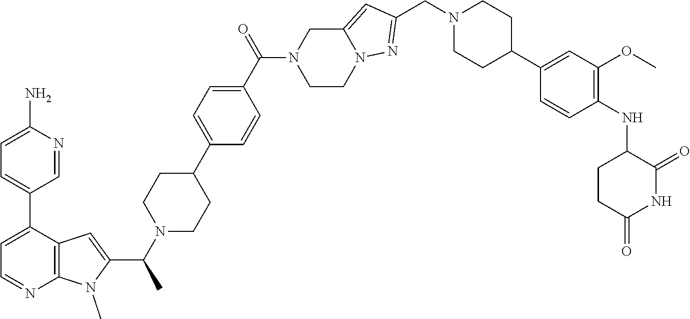 [SMILES: COc1cc(C2CCN(Cc3cc4n(n3)CCN(C(=O)c3ccc(C5CCN([C@@H](C)c6cc7c(-c8ccc(N)nc8)ccnc7n6C)CC5)cc3)C4)CC2)ccc1NC1CCC(=O)NC1=O]

### Example 2c44. 3-((4-(1-((5-(4-(1-((S)-1-(4-(6-aminopyridin-3-yl)-1-methyl-1H-pyrrolo[2,3-b] pyridin-2-yl)ethyl)piperidin-4-yl)benzoyl)-4,5,6,7-tetrahydropyrazolo[1,5-a]pyrazin-2-yl)methyl)piperidin-4-yl)-2-methoxyphenyl)amino)piperidine-2,6-dione

Int. 2C84 (40 mg) was added to a mixture of Int. 3E83 (30 mg) and HATU (50 mg) in DMF/Et₃N (89:3, 2.07 mL) and stirred for 12 h at RT. The mixture was concentrated and HPLC-purified to give Example 2c44 (5 mg).

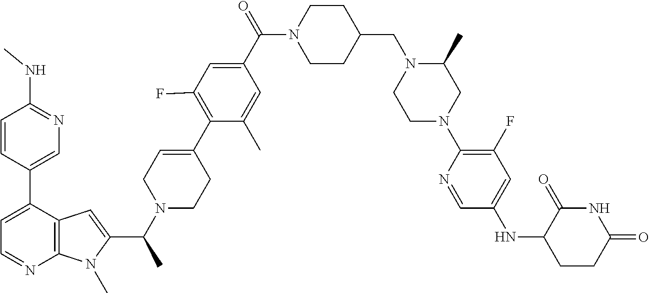 [SMILES: CNc1ccc(-c2ccnc3c2cc([C@H](C)N2CC=C(c4c(C)cc(C(=O)N5CCC(CN6CCN(c7ncc(NC8CCC(=O)NC8=O)cc7F)C[C@@H]6C)CC5)cc4F)CC2)n3C)cn1]

### Example 2c45. 3-((5-fluoro-6-((S)-4-((1-(3-fluoro-5-methyl-4-(1-((S)-1-(1-methyl-4-(6-(methyl-amino)pyridin-3-yl)-1H-pyrrolo[2,3-b]pyridin-2-yl)ethyl)-1,2,3,6-tetrahydropyridin-4-yl)benzoyl)-piperidin-4-yl)methyl)-3-methylpiperazin-1-yl)pyridin-3-yl)amino)piperidine-2,6-dione

Example 2c45 (35 mg) was prepared similarly to Example XXX258 from Ints. 3C38/3E300 (0.10 g/0.12 g).

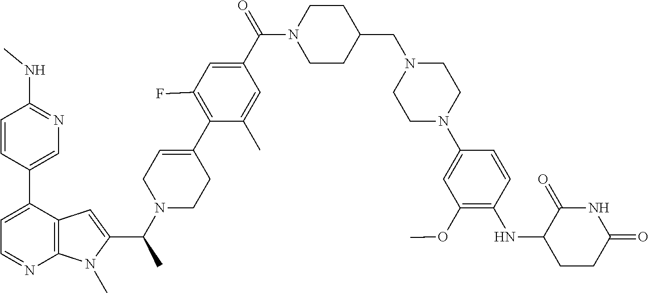 [SMILES: CNc1ccc(-c2ccnc3c2cc([C@H](C)N2CC=C(c4c(C)cc(C(=O)N5CCC(CN6CCN(c7ccc(NC8CCC(=O)NC8=O)c(OC)c7)CC6)CC5)cc4F)CC2)n3C)cn1]

### Example 2c46. 3-((4-(4-((1-(3-fluoro-5-methyl-4-(1-((S)-1-(1-methyl-4-(6-(methylamino)pyridin-3-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)-2-methoxyphenyl)amino)piperidine-2,6-dione

Example 2c46 (29 mg) was prepared similarly to Example 2c35 from Ints. 3C38/2C79 (0.10 g/0.12 g).

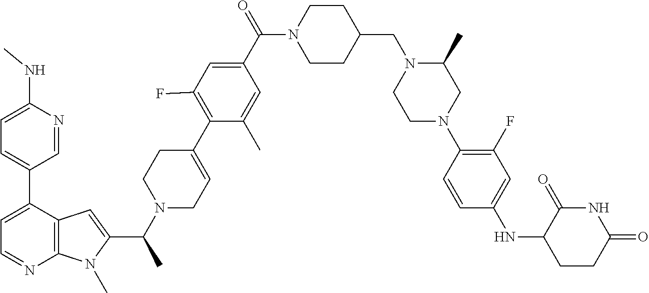 [SMILES: CNc1ccc(-c2ccnc3c2cc([C@H](C)N2CC=C(c4c(C)cc(C(=O)N5CCC(CN6CCN(c7ccc(NC8CCC(=O)NC8=O)cc7F)C[C@@H]6C)CC5)cc4F)CC2)n3C)cn1]

### Example 2c47. 3-((3-fluoro-4-((S)-4-((1-(3-fluoro-5-methyl-4-(1-((S)-1-(1-methyl-4-(6-(methylamino)pyridin-3-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)phenyl)amino)piperidine-2,6-dione

Example 2c47 (45 mg) was prepared similarly to Example 2c35 from Ints. 3C38/2F21 (0.12 g/0.14 g).

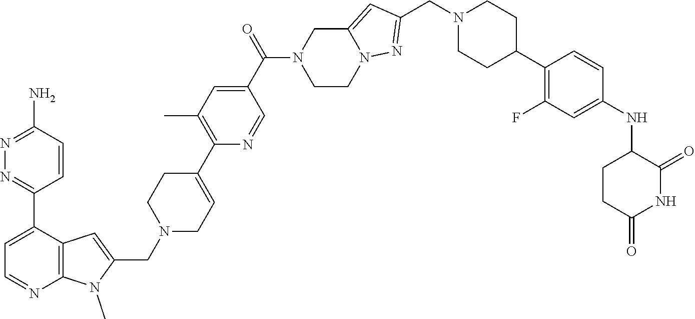 [SMILES: Cc1cc(C(=O)N2CCn3nc(CN4CCC(c5ccc(NC6CCC(=O)NC6=O)cc5F)CC4)cc3C2)cnc1C1=CCN(Cc2cc3c(-c4ccc(N)nn4)ccnc3n2C)CC1]

### Example 2c48. 3-((4-(1-((5-(1′-((4-(6-aminopyridazin-3-yl)-1-methyl-1H-pyrrolo[2,3-b]pyridin-2-yl)methyl)-3-methyl-1′, 2′, 3′, 6′-tetrahydro-[2,4′-bipyridine]-5-carbonyl)-4,5,6,7-tetrahydropyrazolo[1,5-a]pyrazin-2-yl)methyl)piperidin-4-yl)-3-fluorophenyl)amino)piperidine-2,6-dione

Example 2c48 (7 mg) was prepared similarly to Example 2c37 (3 mg) from Int. 2C74 (34 mg).

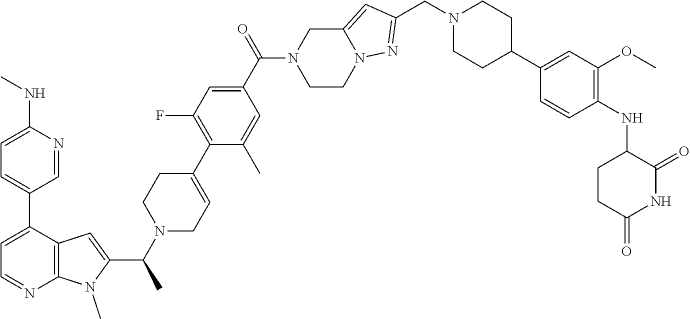 [SMILES: CNc1ccc(-c2ccnc3c2cc([C@H](C)N2CC=C(c4c(C)cc(C(=O)N5CCn6nc(CN7CCC(c8ccc(NC9CCC(=O)NC9=O)c(OC)c8)CC7)cc6C5)cc4F)CC2)n3C)cn1]

### Example 2c49. 3-((4-(1-((5-(3-fluoro-5-methyl-4-(1-((S)-1-(1-methyl-4-(6-(methylamino)pyridin-3-yl)-1H-pyrrolo[2,3-b]pyridin-2-yl)ethyl)-1,2,3,6-tetrahydropyridin-4-yl)benzoyl)-4,5,6,7-tetrahydropyrazolo[1,5-a]pyrazin-2-yl)methyl)piperidin-4-yl)-2-methoxyphenyl)amino)piperidine-2,6-dione

Example 2c49 (20 mg) was prepared similarly to Example 2c35 from Ints. 3C38/2C84 (0.10 g/0.13 g).

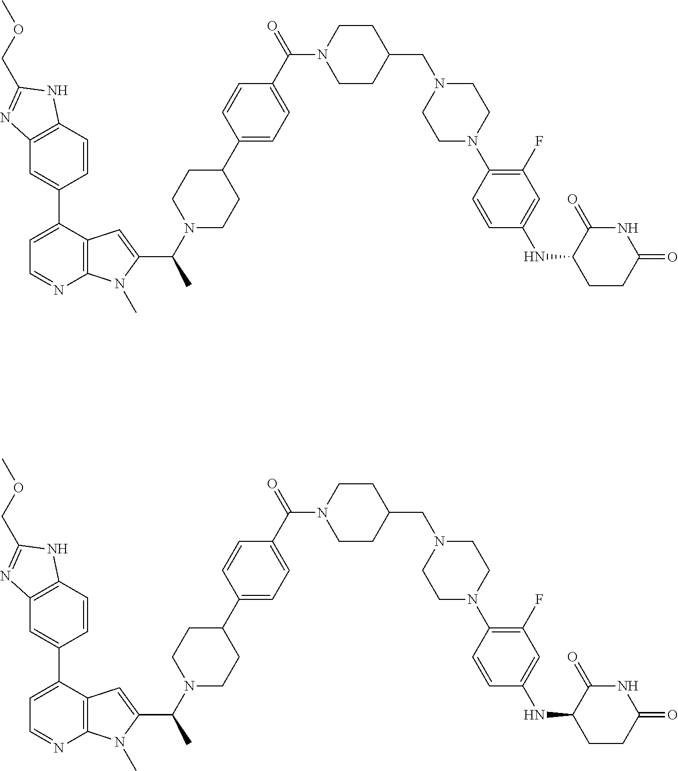 [SMILES: COCc1nc2cc(-c3ccnc4c3cc([C@H](C)N3CCC(c5ccc(C(=O)N6CCC(CN7CCN(c8ccc(N[C@H]9CCC(=O)NC9=O)cc8F)CC7)CC6)cc5)CC3)n4C)ccc2[nH]1 ; COCc1nc2cc(-c3ccnc4c3cc([C@H](C)N3CCC(c5ccc(C(=O)N6CCC(CN7CCN(c8ccc(N[C@@H]9CCC(=O)NC9=O)cc8F)CC7)CC6)cc5)CC3)n4C)ccc2[nH]1]

### Example 2c52 and 2c53. (R)-3-((3-fluoro-4-(4-((1-(4-(1-((S)-1-(4-(2-(methoxymethyl)-1H-benzo[d] imidazol-5-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione and(S)-3-((3-fluoro-4-(4-((1-(4-(1-((S)-1-(4-(2-(methoxymethyl)-1H-benzo[d]imidazol-5-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione

Example 2c16 (0.80 g) was resolved by HPLC using a Chiralpak IE 250×30 mm 5 μm column with an eluent of 35% ACN, 35% IPA and 30% DCM (28 mL/min) to give Example 2c52 (170 mg, first peak) and Example 2c53 (97 mg, second peak). The absolute configurations of these compounds at the glutarimide chiral centers were not determined.

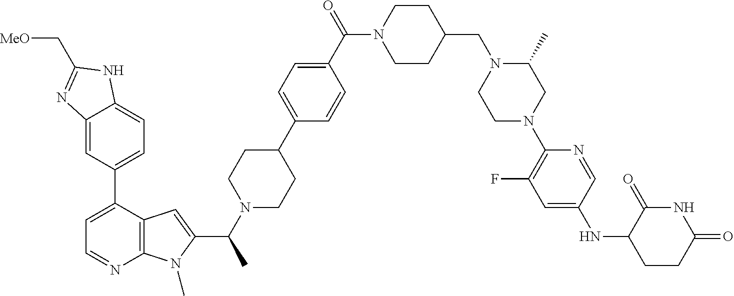 [SMILES: COCc1nc2cc(-c3ccnc4c3cc([C@H](C)N3CCC(c5ccc(C(=O)N6CCC(CN7CCN(c8ncc(NC9CCC(=O)NC9=O)cc8F)C[C@H]7C)CC6)cc5)CC3)n4C)ccc2[nH]1]

### Example 3t5. 3-((5-fluoro-6-((R)-4-((1-(4-(1-((S)-1-(4-(2-(methoxymethyl)-1H-benzo[d] imidazol-5-yl)-1-methyl-1H-3-((5-fluoro-6-((S)-4-((1-(4-(1-((S)-1-(4-(2-(methoxymethyl)-1H-benzo[d] imidazol-5-yl)-1-methyl-1H-pyrrolo[2,3-b]pyridin-2-yl)ethyl)piperidin-4-yl)benzoyl)piperidin-4-yl)methyl)-3-methylpiperazin-1-yl)pyridin-3-yl)amino)piperidine-2,6-dione

Ints. 3E300/3E120 (0.2 g/0.24 g) and PyBrop (0.31 g) were stirred ON in THF/DIPEA (18.5:1, 10.5 mL) at 0° C. to RT, concentrated, triturated in water, and HPLC-purified to give Example 3t5(42 mg).

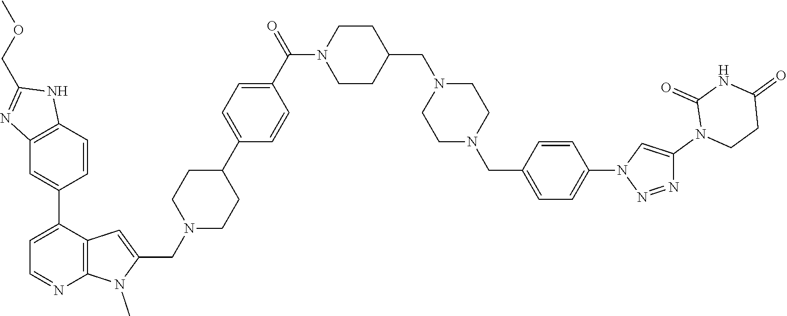 [SMILES: COCc1nc2cc(-c3ccnc4c3cc(CN3CCC(c5ccc(C(=O)N6CCC(CN7CCN(Cc8ccc(-n9cc(N%10CCC(=O)NC%10=O)nn9)cc8)CC7)CC6)cc5)CC3)n4C)ccc2[nH]1]

### Example 2c56. 1-(1-(4-((4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperazin-1-yl)methyl)-phenyl)-1H-1,2,3-triazol-4-yl) dihydropyrimidine-2,4 (1H,3H)-dione

Example 2c56 (10 mg) was prepared similarly to Example 2c44 from Ints. 3E261/2T23 (22 mg/26 mg).

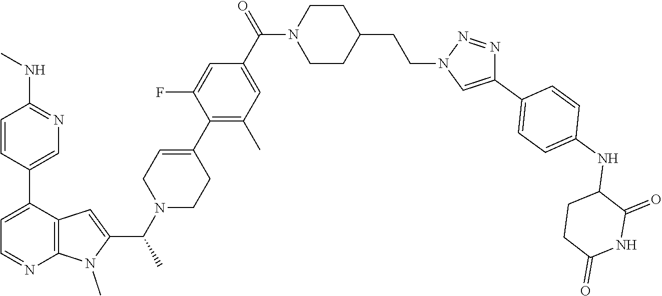 [SMILES: CNc1ccc(-c2ccnc3c2cc([C@@H](C)N2CC=C(c4c(C)cc(C(=O)N5CCC(CCn6cc(-c7ccc(NC8CCC(=O)NC8=O)cc7)nn6)CC5)cc4F)CC2)n3C)cn1]

### Example 2c57. 3-((4-(1-(2-(1-(3-fluoro-5-methyl-4-(1-((R)-1-(1-methyl-4-(6-(methylamino)pyridin-3-yl)-1H-pyrrolo[2,3-b]pyridin-2-yl)ethyl)-1,2,3,6-tetrahydropyridin-4-yl)benzoyl)piperidin-4-yl)ethyl)-1H-1,2,3-triazol-4-yl)phenyl)amino)piperidine-2,6-dione

Example 2c57 (70 mg) was prepared similarly to Example 2c45 from Ints. 2T25/2T29 (0.12 g/0.19 g).

Characterization data

| Example | Method | RT | Purity | MassCalcd | MassObs |
|---|---|---|---|---|---|
| 3t5 | 8 | 3.99 | 97.6 | 923.4 | 924.6 |
| 2t5 | 11 or 12 | 2.07 | 96 | 788.3 | 789.3 |
| 2t4 | 11 or 12 | 1.81 | 95* | 871.4 | 872.4 |
| 2t3 | 11 or 12 | 1.71 | 85 | 833.4 | 834.4 |
| 2t2 | 11 or 12 | 1.82 | 95* | 860.4 | 861.4 |
| 2t1 | 11 or 12 | 1.82 | 94 | 879.4 | 880.4 |
| 2s2 | 11 or 12 | 1.64 | 95* | 840.4 | 841.4 |
| 2s1 | 11 or 12 | 1.77 | 90* | 846.4 | 847.4 |
| 2r1 | 11 or 12 | 1.72 | 95* | 916.4 | 917.4 |
| 2q9 | 8 | 3.91 | 97.6 | 913.4 | 914.5 |
| 2q8 | 10 | 12.5 | 96.5 | 932.4 | 933.6 |
| 2q7 | 9 | 9.51 | 82.8 | 931.4 | 932.6 |
| 2q6 | 8 | 3.69 | 95.2 | 878.4 | 879.5 |
| 2q5 | 9 | 9.74 | 97.1 | 877.4 | 878.6 |
| 2q4 | 8 | 3.72 | 98.4 | 863.4 | 864.5 |
| 2q3 | 2 | 1.26 | 97.1 | 887.4 | 888.5 |
| 2q2 | 9 | 9.41 | 90.7 | 888.4 | 889.6 |
| 2q15 | 8 | 3.65 | 96.5 | 878.4 | 879.5 |
| 2q14 | 8 | 3.61 | 95.4 | 879.4 | 880.5 |
| 2q12 | 8 | 3.84 | 99.2 | 967.4 | 968.5 |
| 2q11 | 8 | 3.85 | 99.2 | 877.4 | 878.5 |
| 2q10 | 8 | 3.85 | 99.6 | 877.4 | 878.5 |
| 2q1 | 9 | 8.94 | 95.0 | 864.4 | 865.6 |
| 2p3 | 11 or 12 | 2.37 | 90* | 1013. | 1014. |
| 2p2 | 11 or 12 | 1.78 | 95* | 945.5 | 946.5 |
| 2p1 | 11 or 12 | 1.93 | 95* | 969.5 | 970.5 |
| 2o2 | 11 or 12 | 1.87 | 95* | 853.4 | 854.4 |
| 2o1 | 11 or 12 | 1.72 | 95* | 829.4 | 830.4 |
| 2n9 | 11 or 12 | 0.70 | 95* | 841.5 | 842.6 |
| 2n8 | 11 or 12 | 1.84 | 98* | 865.4 | 866.4 |
| 2n7 | 11 or 12 | 1.85 | 98* | 865.4 | 866.4 |
| 2n6 | 11 or 12 | 1.75 | 95* | 895.4 | 896.4 |
| 2n5 | 11 or 12 | 2.2 | 95* | 827.4 | 828.4 |
| 2n4 | 11 or 12 | 1.64 | 95 | 827.4 | 828.4 |
| 2n3 | 11 or 12 | 1.75 | 90 | 895.4 | 896.4 |
| 2n2 | 11 or 12 | 1.81 | 95 | 851.4 | 852.4 |
| 2n14 | 2 | 1.07 | 96.1 | 855.4 | 856.5 |
| 2n13 | 8 | 4.1 | 98.1 | 855.4 | 856.5 |
| 2n12 | 3 | 3.82 | 97.8 | 909.4 | 910.6 |
| 2n11 | 2 | 1.19 | 91.3 | 909.4 | 910.4 |
| 2n10 | 8 | 3.9 | 98.5 | 841.4 | 842.5 |
| 2n1 | 11 or 12 | 0.70 | 95* | 833.4 | 834.5 |
| 2m3 | 8 | 4.3 | 98.6 | 943.4 | 944.5 |
| 2m2 | 4 | 2.25 | 97.2 | 875.4 | 876.6 |
| 2m1 | 11 or 12 | 1.82 | 97 | 849.4 | 850.4 |
| 2l1 | 11 or 12 | 1.81 | 90 | 832.4 | 833.4 |
| 2k3 | 11 or 12 | 1.69 | 85 | 834.4 | 835.4 |
| 2k2 | 11 or 12 | 1.69 | 90 | 834.4 | 835.4 |
| 2k1 | Purity by | 1.76 | 95 | 834.4 | 835.4 |
| | MS | | | | |
| 2j2 | 11 or 12 | 1.87 | 95 | 839.4 | 840.4 |
| 2j1 | 11 or 12 | 2.05 | 93 | 857.4 | 858.4 |
| 2i7 | 8 | 4.41 | 98.8 | 890.4 | 891.6 |
| 2i6 | 3 | 3.63 | 96.9 | 856.4 | 857.5 |
| 2i5 | 8 | 4.33 | 99.2 | 958.4 | 959.5 |
| 2i4 | 4 | 2.35 | 91.0 | 924.4 | 925.6 |
| 2i3 | 2 | 1.11 | 95.0 | 847.4 | 848.4 |
| 2i2 | 3 | 3.84 | 95.3 | 915.4 | 916.7 |
| 2i1 | 11 or 12 | 1.83 | 95 | 880.4 | 881.4 |
| 2h3 | 11 or 12 | 2.47 | 95* | 1012. | 1013. |
| 2h2 | 11 or 12 | 1.79 | 95* | 944.5 | 945.5 |
| 2h1 | 11 or 12 | 1.93 | 95* | 968.5 | 969.5 |
| 2g63 | 41 | 0.735 | 100 | 840.5 | 839.4 |
| 2g1 | 11 or 12 | 1.82 | 95* | 871.4 | 71.46 |
| 2f9 | 2 | 1.5 | 98.1 | 908.4 | 909.5 |
| 2f8 | 11 or 12 | 1.84 | 97 | 864.4 | 865.4 |
| 2f7 | 11 or 12 | 1.78 | 95 | 908.4 | 909.4 |
| 2f6 | 11 or 12 | 1.67 | 95 | 840.4 | 841.4 |
| 2f5 | 11 or 12 | 1.84 | 97 | 864.4 | 865.4 |
| 2f4 | 11 or 12 | 1.78 | 95 | 908.4 | 909.4 |
| 2f3 | 11 or 12 | 1.68 | 98 | 840.4 | 841.4 |
| 2f2 | 11 or 12 | 1.7 | 98 | 854.4 | 855.4 |
| 2f13 | 9 | 10.3 | 95.9 | 922.5 | 923.5 |
| 2f12 | 9 | 10.3 | 97.6 | 922.5 | 923.5 |
| 2f11 | 8 | 4.41 | 92.6 | 908.4 | 909.5 |
| 2f10 | 8 | 4.4 | 96.9 | 908.4 | 909.6 |
| 2f1 | 8 | 4.07 | 99.4 | 922.5 | 923.8 |
| 2e2 | 11 or 12 | 1.61 | 95 | 854.4 | 855.4 |
| 2e1 | 11 or 12 | 1.79 | 95 | 878.4 | 879.4 |
| 2d1 | 11 or 12 | 1.74 | 98 | 862.4 | 863.4 |
| 2c9 | 11 or 12 | 1.68 | 97 | 838.4 | 839.4 |
| 2c8 | 11 or 12 | 1.8 | 90* | 851.4 | 852.4 |
| 2c7 | 11 or 12 | 1.63 | 90* | 840.4 | 841.4 |
| 2c6 | 2 | 1.31 | 98.9 | 850.4 | 851.4 |
| 2c57 | 34 | 4.05 | 98.08 | 863.4 | 864.48 |
| 2c56 | 11 or 12 | | 95 | 943.5 | 944.5 |
| 2c55 | 21 | 3 | 98.37 | 855.5 | 856.54 |
| 2c54 | 26 | 3.77 | 97.59 | 854.5 | 855.44 |
| 2c53 | 10 | 10.03 | 98.68 | 908.5 | 909.65 |
| 2c52 | 10 | 10.01 | 98.89 | 908.5 | 909.65 |
| 2c5 | 2 | 1.32 | 96.5 | 850.4 | 851.5 |
| 2c49 | 34 | 4.12 | 95.05 | 933.5 | 934.56 |
| 2c48 | 41 | 0.65 | 93.76 | 877.4 | 876 |
| 2c47 | 17 | 5.48 | 94.35 | 898.5 | 899.45 |
| 2c46 | 21 | 3.05 | 97.68 | 896.5 | 897.49 |
| 2c45 | 21 | 3.1 | 99.11 | 899.5 | 900.52 |
| 2c44 | 41 | 0.795 | 96.94 | 889.5 | 891.4 |
| 2c43 | 10 | 10.09 | 96.67 | 891.5 | 892.53 |
| 2c42 | 10 | 10.1 | 96.58 | 891.5 | 892.53 |
| 2c41 | 10 | 10.1 | 97.49 | 891.5 | 892.59 |
| 2c40 | 21 | 4.15 | 95.71 | 852.5 | 853.51 |
| 2c4 | 11 or 12 | 1.75 | 95* | 894.4 | 895.4 |
| 2c39 | 29 | 1.33 | 97.65 | 827.4 | 828.62 |
| 2c38 | 41 | 0.556 | 94.47 | 863.4 | 862 |
| 2c37 | 41 | 0.68 | 94.38 | 862.4 | 861.2 |
| 2c34 | 11 or 12 | 1.83 | 97 | 868.4 | 869.4 |
| 2c33 | 4 | 2.12 | 95.3 | 840.4 | 841.5 |
| 2c32 | 2 | 1.35 | 98.0 | 870.4 | 871.5 |
| 2c30 | 8 | 4.04 | 97.4 | 858.4 | 859.5 |
| 2c3 | 11 or 12 | 1.77 | 97* | 844.4 | 845.4 |
| 2c28 | 4 | 2.59 | 92.3 | 894.4 | 895.6 |
| 2c27 | 3 | 3.67 | 96.3 | 841.4 | 842.5 |
| 2c26 | 2 | 1.13 | 95.1 | 884.4 | 885.5 |
| 2c24 | 4 | 2.56 | 93.6 | 858.4 | 859.5 |
| 2c23 | 8 | 4.14 | 97.4 | 858.4 | 859.5 |
| 2c22 | 3 | 3.96 | 95.4 | 865.4 | 866.6 |
| 2c21 | 4 | 2.29 | 96.7 | 922.5 | 923.7 |
| 2c20 | 8 | 4.07 | 99.6 | 840.4 | 841.5 |
| 2c2 | 11 or 12 | 1.64 | 97* | 826.4 | 827.4 |
| 2c19 | 8 | 4.02 | 97.4 | 840.4 | 841.5 |
| 2c18 | 8 | 4.06 | 98.3 | 922.5 | 923.5 |
| 2c17 | 11 or 12 | 1.86 | 95 | 864.4 | 865.4 |
| 2c16 | 4 | 2.16 | 99.8 | 908.4 | 909.5 |
| 2c15 | 11 or 12 | 2.32 | 95* | 852.4 | 853.4 |
| 2c14 | 9 | 10.1 | 96.9 | 840.4 | 841.5 |
| 2c13 | 11 or 12 | 2.3 | 95* | 840.4 | 841.4 |
| 2c12 | 11 or 12 | 1.67 | 95* | 840.4 | 841.4 |
| 2c11 | 11 or 12 | 1.67 | 95* | 840.4 | 841.4 |
| 2c10 | 11 or 12 | 1.67 | 95* | 840.4 | 841.4 |
| 2c1 | 11 or 12 | 1.79 | 97 | 850.4 | 851.4 |
| 2b3 | 11 or 12 | 1.74 | 90* | 894.4 | 895.4 |
| 2b2 | 11 or 12 | 1.81 | 90* | 850.4 | 851.4 |
| 2b1 | 11 or 12 | 1.79 | 95 | 832.4 | 833.4 |
| 2a2 | 11 or 12 | 1.77 | 88 | 879.4 | 880.4 |
| 2a1 | 11 or 12 | 1.79 | 98 | 878.4 | 879.4 |

Method refers to one of LC/MS methods 1-41
RT = retention time in min
Purity is based on LC/MS (UV) or by ¹H NMR (if indicated with *)

### Example 3. Pharmacology Data for the Compounds of the Disclosure

| | | | STAT6 | STAT6 |
|---|---|---|---|---|
| | Eotaxin-3 | STAT6 SPR | degradation | degradation |
| Example | EC₅₀ (nM) | K_{d} (nM) | DC₅₀ (nM) | Eₘₐₓ (%) |
| 2a1 | | 224 | 4.9 | |
| 2a2 | | 61.2 | | 60-70 |
| 2b1 | | | 19.2 | |
| 2b2 | | | | 70-80 |
| 2b3 | | | | 70-80 |
| 2c1 | 556 | 193 | | 70-80 |
| 2c10 | | | 14.1 | |
| 2c11 | | | 11.2 | |
| 2c12 | | | 17.7 | |
| 2c13 | | | | <20 |
| 2c14 | 65 | | 6.6 | |
| 2c15 | | | | 80-90 |
| 2c16 | 22.6 | | 1.2 | |
| 2c17 | | | 2.7 | |
| 2c18 | 39 | | 4.8 | |
| 2c19 | 109 | | 10.5 | |
| 2c2 | | | | 50-60 |
| 2c20 | 82.8 | | 9.6 | |
| 2c21 | | | 4.4 | |
| 2c22 | 153 | | 28.2 | |
| 2c23 | 49.2 | | 9.6 | |
| 2c24 | 792 | | 44.7 | |
| 2c26 | 188 | | | 60-70 |
| 2c27 | 169 | | | 70-80 |
| 2c28 | 95.3 | | | 70-80 |
| 2c3 | | <20 | | <20 |
| 2c30 | 507 | | | 30-40 |
| 2c32 | 67.9 | | 16.5 | |
| 2c33 | 136 | | 22.5 | |
| 2c34 | | | | 70-80 |
| 2c37 | | | | <20 |
| 2c38 | | | | <20 |
| 2c39 | | | | <20 |
| 2c4 | | | | 70-80 |
| 2c40 | 112 | | | |
| 2c41 | | | 3.3 | |
| 2c42 | | | 5.4 | |
| 2c43 | | | 1.6 | |
| 2c44 | | | 0.7 | |
| 2c45 | 104 | | 21.3 | |
| 2c46 | 58.4 | | 21.4 | |
| 2c47 | 197 | | | |
| 2c48 | | | | <20 |
| 2c49 | 3.0 | | | |
| 2c5 | | | | 70-80 |
| 2c52 | 30.7 | | 3.9 | |
| 2c53 | 30.9 | | 4.7 | |
| 2c54 | 265 | | | |
| 2c55 | 247 | | | |
| 2c56 | | | | <20 |
| 2c57 | | | | <20 |
| 2c6 | | | 22.5 | |
| 2c7 | | | | 30-40 |
| 2c8 | | 40-50 | | <20 |
| 2c9 | | | | 50-60 |
| 2d1 | | | | 50-60 |
| 2e1 | | <20 | | <20 |
| 2e2 | | | | <20 |
| 2f1 | 25 | | 3.4 | |
| 2f10 | 385 | | | |
| 2f11 | 406 | | | |
| 2f12 | 19.7 | | 4.1 | |
| 2f13 | 15.6 | | 2.5 | |
| 2f2 | 37 | | 6.8 | |
| 2f3 | | | | 20-30 |
| 2f4 | | | | 50-60 |
| 2f5 | | | | 50-60 |
| 2f6 | | | 38.8 | |
| 2f7 | | | 6.6 | |
| 2f8 | | | 19.1 | |
| 2f9 | 249 | | 69.2 | |
| 2g1 | | | | <20 |
| 2g63 | | | 48.3 | |
| 2h1 | | | 66.3 | |
| 2h2 | | | 133 | |
| 2h3 | | | 54.8 | |
| 2i1 | | | | 40-50 |
| 2i2 | | | | 30-40 |
| 2i3 | | | | 30-40 |
| 2i4 | | | 7.6 | |
| 2i5 | | | 34 | |
| 2i6 | | | | 70-80 |
| 2i7 | | | | 40-50 |
| 2j1 | | | | 70-80 |
| 2j2 | | | | 40-50 |
| 2k1 | | | 28.8 | |
| 2k2 | | | | 30-40 |
| 2k3 | | | | 30-40 |
| 2l1 | | | | 60-70 |
| 2m1 | | 466 | | 60-70 |
| 2m2 | 118 | | | 60-70 |
| 2m3 | | | 23.5 | |
| 2n1 | | | 15.2 | |
| 2n10 | 131 | | | 70-80 |
| 2n11 | | | | 70-80 |
| 2n12 | 40.9 | | | 70-80 |
| 2n13 | | | 30 | 79.3 |
| 2n14 | | | 49 | |
| 2n2 | | | 28.6 | |
| 2n3 | | | 19.8 | |
| 2n4 | | | | 70-80 |
| 2n5 | | | | 60-70 |
| 2n6 | | | 11.7 | |
| 2n7 | | | 18 | |
| 2n8 | | | 6.5 | |
| 2n9 | 141 | | | 70-80 |
| 2o1 | | | | 50-60 |
| 2o2 | | | | 60-70 |
| 2p1 | | | 36.9 | |
| 2p2 | | | 61 | |
| 2p3 | | | 20.6 | |
| 2q1 | | | | 40-50 |
| 2q10 | 3.2 | | 1.0 | |
| 2q11 | 5.9 | | 0.8 | |
| 2q12 | | | 12.9 | |
| 2q14 | 86.7 | | 24.7 | |
| 2g15 | 37.8 | | | |
| 2q2 | | | | 70-80 |
| 2q3 | 11.3 | | 2.7 | |
| 2q4 | 73.5 | | | 70-80 |
| 2q5 | 2.3 | | 1.0 | |
| 2q6 | 19.9 | | 5.6 | |
| 2q7 | 2.4 | | 2.6 | |
| 2q8 | 25.1 | | | |
| 2q9 | 26.7 | | 2.9 | |
| 2r1 | | | 0.35 | |
| 2s1 | | | | 60-70 |
| 2s2 | | | | 40-50 |
| 2t1 | | 105 | 8.1 | |
| 2t2 | | | | <20 |
| 2t3 | | | | 40-50 |
| 2t4 | | <20 | | <20 |
| 2t5 | | | | 30-40 |
| 3t5 | 59.5 | | 6.0 | |

Eotaxin-3 EC₅₀ values provided for test compounds with >80% Eₘₐₓ
STAT6 degradation DC₅₀ values provided for test compounds with >80% Eₘₐₓ except 2c15 for which Eₘₐₓ is provided
STAT6 degradation Eₘₐₓ values provided for test compounds with <80% Eₘₐₓ
Data is provided as geometric mean values across multiple assay runs and compound batches to the extent possible

### Surface Plasmon Resonance Assay

SPR was used to confirm and quantify binding of compounds to human STAT6. Biacore instruments from the 8K-series (Cytiva) were used, and measurements were performed at 25° C. Human STAT6 (truncated, aa122-658, with N-terminal His-tag, expressed in insect cells) was immobilized in the active flow cells on a CM5 chip using amine-coupling (default settings) at 10 μg/ml in 10 mM acetate buffer pH 5.5, at a contact time of 120-420 sec. and a flow rate of 10 pl/min. Reference flow cells were deactivated using blank immobilization. PBS-P+ (Cytiva) supplemented with 2% DMSO was used as running buffer, and solvent correction was applied. Compounds were tested by multi-cycle kinetics injections using a contact time of 60 sec., dissociation time of up to 300 sec., and a flow rate of 30 pl/min.

Solvent-corrected, reference-subtracted and blank-corrected data was processed and binding isotherms were fitted according to a single report point from each sensorgram (Standard: Late binding (5 sec. before injection end); alternative: Earlier report point according to binding profile). Data fitting was done both with unfixed Rmax and with Rmax fixed to the theoretical value* assuming 1:1 binding to allow processing of data from both weak and strong binders as well as to evaluate binding stoichiometry.

Positive controls were run at the beginning, during, and at the end of the screen to evaluate surface activity. Generally surface activity was only affected to a minor degree, and no corrections were applied.

*Equation for calculation of the theoretical Rmax (Theoretical Rmax=(MW Analyte/MW Ligand) x immobilization level).

The sensorgrams for Examples 2a1, 2t1, 2m1, and 2a2 are shown below (where RU is plotted vs. time in seconds; dotted lines/points were excluded from the analysis due to effects from non-specific binding).

### STAT6 Degrader Assay

This was a HiBit based assay. A HeLa cell line stably transfected with a small protein, LgBit (Large Bit) was obtained from Promega. These cells express endogenous STAT6. A small 11 amino acid gene sequence (HiBiT) was inserted in the STAT6 gene using CRISPR technology. Once STAT6 protein is expressed in the cells, the HiBiT sequence will bind the LgBit in the cells resulting in a functional NanoBiT protein, giving a luminescent tag to STAT6.

Single cell clones were prepared and the clone 4.14 was selected for the assay based on that clone showing high luminescence, high STAT6 phosphorylation upon stimulation with IL-4, and inhibition of luminescence when cells were treated with STAT6 siRNA.

HeLa LgBiT clone 4.14 cells were suspended in DMEM with 10% FCS, 1% Pen/Strep, Na-pyrovate and Hygromycin B, at a concentration of 1.25 * 105 cells pr mL. 5000 cells were added pr well of a 384 well plate and 20 nL of compound was added by Echo® liquid handler. NN6394A (terfenadine, a toxic compound) was added to control wells at 50 M concentration to establish the assay window. In these wells the cells are dead which equals 100% degradation of STAT6.

After 4-24 h of incubation at 37° C. under 5% CO₂/95% air, 5 L substrate (Nano-GLO®) was added to the wells and the plates were centrifuged and incubated for 0.5 h.

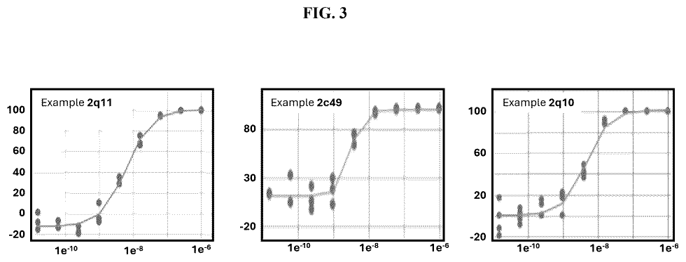

The luminescence in the wells as a measure of non-degraded STAT6 was measured in a plate reader (BMG Pherastar FSX). The measured DC₅₀ (in nM) was determined and reported in FIG. 3.

The dose-response curves for Examples 2a2, 2t1, 2 h3, and 2r1 are shown below (where percent degradation is plotted vs. test compound concentration in M on a log-scale).

### Human Whole Blood Eotaxin-3 Assay

This was a human whole blood assay using recombinant human Interleukin 4 to stimulate the blood and measuring the ability of the test compounds to inhibit eotaxin-3 release. The biological activities of the test compounds have been tested in human whole blood stimulated with IL-4 measuring Eotaxin-3 release. The test compounds were added to 384 well clear flat-bottom plate in a 4-fold serial dilution in triplicates using a liquid handler. Human whole blood stabilized in lithium-heparin tubes was added in a volume of 65 uL per well, resulting in a final DMSO concentration of 0.5%. The plate is incubated for 2 h at 37° C. under 5% CO₂/95% air. Subsequently, 5 μL of recombinant human IL-4 (R&D Systems, cat #204-ILB) was added to the wells to a final concentration of 400 pM. The plate was incubated for 48 h at 37° C. under 5% CO₂/95% air. The plate was spun down for 10 minutes at 500×g and 20 uL supernatant was harvested and transferred to a 384 well v-bottom plate using a liquid handler and stored at −20° C. until further analysis. 10 uL supernatant was used to measure the level of Eotaxin-3 (CCL26) using Human Eotaxin-3 MSD kit from Mesocsale (Cat #K251UEK-4). The assay was performed according to the manufacture instructions.

The data from the human whole blood eotaxin-3 assay is shown below for Examples 2q11, 2110, and 2c49 (where the effect in % is plotted against the test concentration in M on a log-scale).

Unless otherwise defined, all technical and scientific terms used herein have the same meaning as commonly understood by one of ordinary skill in the art to which this disclosure belongs.

The disclosure illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by certain embodiments and optional features, modification, improvement and variation of the disclosure embodied therein herein disclosed may be resorted to by those skilled in the art, and that such modifications, improvements and variations are considered to be within the scope of this disclosure. The materials, methods, and examples provided here are representative of certain embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

### EMBODIMENTS

Embodiment 1. A compound according to formula (I):

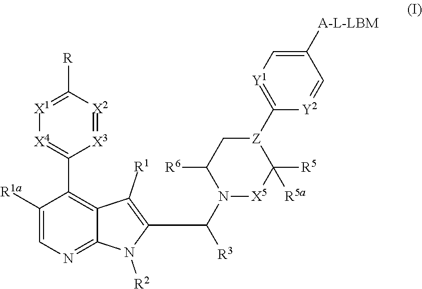

X¹ is selected from CR¹¹ and N; X² is selected from CR¹² and N; X³ is selected from CR¹³ and N, and X⁴ is selected from CR¹⁴ and N, and wherein when X⁴ is N the N may be oxidized to a N-oxide;
X⁵ is CO or CR⁴;
Y¹ is selected from N and CR⁷;
Y² is selected from N and CR⁸;
Z is selected from N and CR¹⁰;
R is NHR⁰;
R⁰ is selected from hydrogen, C₁₋₄alkyl, C₃₋₄cycloalkyl, C₃₋₄cycloalkyl-C₁₋₄alkyl, phenyl-C₁₋₄alkyl, halo-C₁₋₄alkyl, —(CH₂)ₙ—O—R′, —(CH₂)ₙ—CO—C₁₋₄alkyl and —(CH₂)ₙ—CO₂R′, wherein said phenyl is optionally substituted one or more times with substituents independently selected from halogen, C₁₋₄alkyl, halo-C₁₋₄alkyl, C₃₋₄ cycloalkyl, hydroxy, C₁₋₄alkoxy, cyano,—NR′R″, —CON R′R″ and —CO₂R′, wherein R′ and R″ are each independently selected from hydrogen and C₁₋₄alkyl and n is 0, 1 or 2;
R¹ and R^{1a} are independently selected from hydrogen, fluoro and C₁₋₄alkyl;
R² is selected from hydrogen, C₁₋₅alkyl and deuterated C₁₋₄alkyl, wherein said C₁₋₅alkyl may optionally be substituted one or more times with fluoro;
R³ is selected from hydrogen, C₁₋₄alkyl and deuterated C₁₋₄alkyl;
R⁴ and R⁶ are independently selected from hydrogen and C₁₋₄alkyl and C₁₋₄alkoxy, wherein said C₁₋₄alkyl and C₁₋₄alkoxy may optionally be substituted with one or more halogens;
R⁵ is selected from hydrogen, fluoro and C₁₋₄alkyl; wherein said C₁₋₄alkyl may optionally be substituted with one or more halogens, and R^{5a} and R¹⁰ is hydrogen;
R⁵ and R^{5a} are both selected from fluoro and R¹⁰ is hydrogen;
R⁵ and R¹⁰ together form a bond, and R^{5a} is hydrogen;
R⁷ and R⁸ are independently selected from hydrogen, halogen, C₁₋₄alkyl and C₁₋₄alkoxy, wherein said C₁₋₄alkyl and C₁₋₄alkoxy may optionally be substituted with one or more halogen;
R¹, R¹², R¹³ and R¹⁴ are independently selected from hydrogen, halogen, C₁₋₄alkyl, C₃₋₄ cycloalkyl, C₁₋₄alkoxy, hydroxy, cyano, —NR′R″, —CON R′R″, —CO₂R′, —CO—CO₂R′, —CO—(CH₂)ₘOH, tetrazolyl and —SO₂—C₁₋₄alkyl, wherein said C₁₋₄alkyl and C₁₋₄alkoxy, is optionally substituted one or more times with a substituent independently selected from halogen, hydroxy and C₁₋₄alkoxy, wherein m is 1, 2 or 3, and wherein R′ and R″ are each independently selected from hydrogen and C₁₋₄alkyl;
R¹¹ and R⁰ may together form a 5-6 membered heteroaromatic ring containing one or two N atoms, wherein said 5-6 membered heteroaromatic ring containing one or two N atoms is optionally be substituted one or more times with a substituent independently selected from halogen, C₁₋₄alkyl, halo-C₁₋₄alkyl, C₃₋₄ cycloalkyl, hydroxy, C₁₋₄alkoxy, cyano, —NR′R″, —CON R′R″ and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl;
A is selected from CO
L is selected from

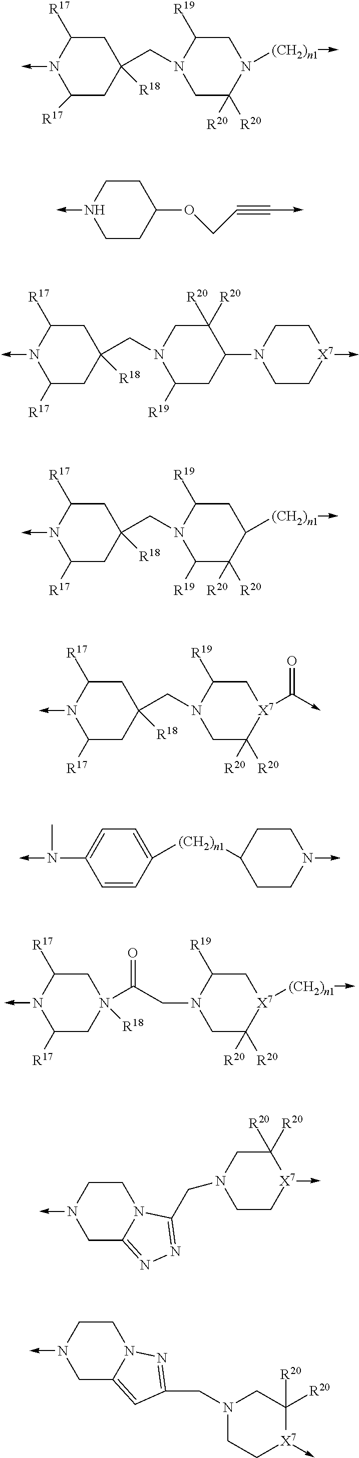

wherein
R¹⁷ is selected from hydrogen and fluoro;
R¹⁸ is selected from hydrogen and fluoro;
R¹⁹ is selected from hydrogen and C₁₋₄alkyl, wherein said C₁₋₄alkyl may optionally be substituted one or more times with fluoro;
R²⁰ is independently selected from hydrogen and fluoro;
n1 is selected from 0 and 1;
X⁷ is selected from N and CH;
and
LBM is selected from

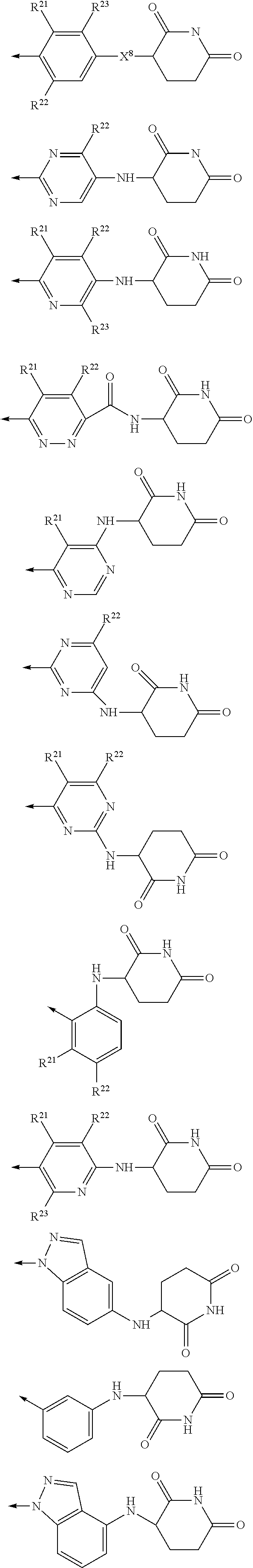

wherein
X⁸ is selected from O and NH;
R²¹ is selected from hydrogen, fluoro, chloro, cyano, C₁₋₄alkyl and trifluoromethyl;
R²² is selected form hydrogen and fluoro,
R²³ is selected from hydrogen, fluoro and C₁₋₄alkoxy;
or pharmaceutically acceptable salts thereof.
Embodiment 2. The compound according to embodiments 1 having the formula (II)

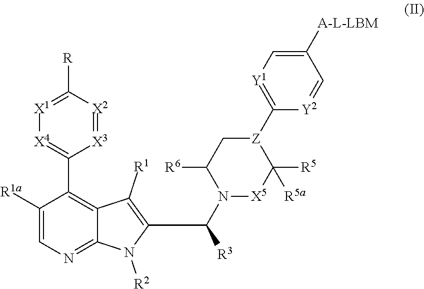

wherein X¹, X², X³, X⁴, X³, Y¹, Y², Z, R, R¹, R^{1a}, R², R⁵, R^{5a}, R⁶, A, L and LBM is as defined in claims 1 and R³ is C₁₋₄alkyl, or pharmaceutically acceptable salts thereof.
Embodiment 3. A compound according to embodiment 1 having the formula (III)

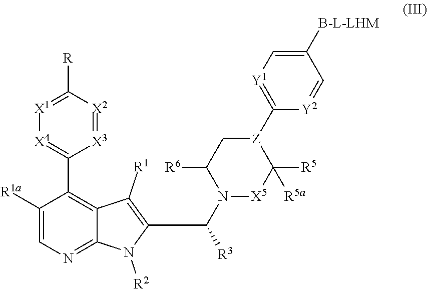

wherein X¹, X², X³, X⁴, X³, Y¹, Y², Z, R, R¹, R^{1a}, R², R⁵, R^{5a}, R⁶, B, L and LBM is as defined in claim 1 and R³ is C₁₋₄alkyl, or pharmaceutically acceptable salts thereof.
Embodiment 4. The compound according to any one of embodiments 1-3, wherein A-L-LBM is selected from

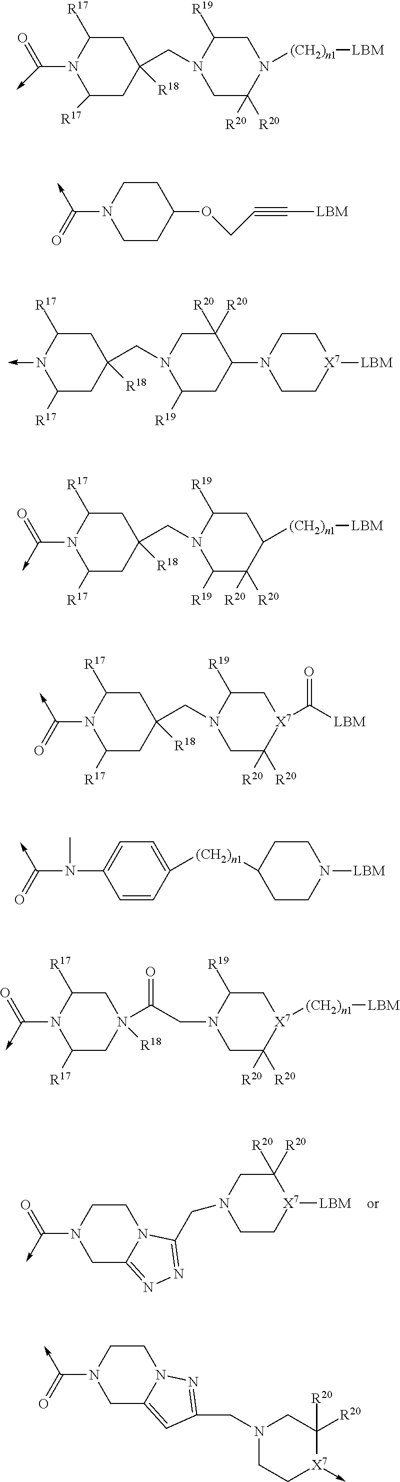

wherein R¹⁷, R¹⁸, R¹⁹, R²⁰, X⁷, n1 and LBM are as defined in claim 1.
Embodiment 5. The compound according to anyone of embodiments 1-3, wherein L is

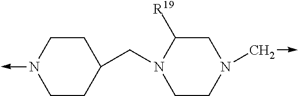

wherein R¹⁹ is as defined in claim 1.
Embodiment 6. The compound according to any one of Embodiments 1-3, wherein L is

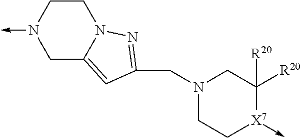

wherein R²⁰ and X⁷ is as defined in claim 1.
Embodiment 7. The compound according to any one of embodiments 1-6 wherein LBM is

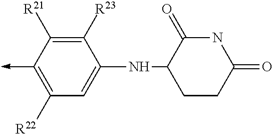

wherein R²¹, R²² and R²³ are as defined in claim 1.
Embodiment 8. The compound according to any one of embodiments 1-3 wherein A-L-LBM is

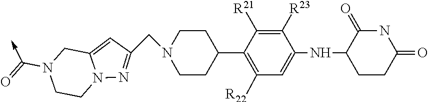

wherein R²¹, R²² and R²³ are as defined in claim 1.
Embodiment 9. The compound according to any one of embodiments 1-3 wherein A-L-LBM is

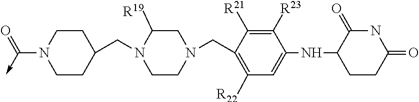

wherein R¹⁹, R²¹, R²² and R²³ are as defined in claim 1.
Embodiment 10. A compound according to any one of embodiment 1-9, wherein X¹ is CR¹¹ and X³ is CR¹³, X⁴ is CR¹⁴ and X² is N.
Embodiment 11. A compound according to embodiment 10 wherein R¹¹, R¹³ and R¹⁴ are independently selected from hydrogen, halogen, C₁₋₄alkyl, C₁₋₄alkoxy, hydroxy, cyano, —NR′R″,—CO₂R′, tetrazole and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.
Embodiment 12. A compound according to embodiment 11, wherein R¹¹, R¹³ and R¹⁴ are independently selected from hydrogen, methyl, methoxy, hydroxy, hydroxymethyl, fluoro, chloro, di- or trifluoromethyl, cyano, methoxycarbonyl, ethoxycarbonyl, —COOH, methylsulfonyl, tetrazolyl and (CH₃)₂N—.
Embodiment 13. The compound according to any one of embodiments 1-9, wherein X¹ is CR¹¹, X² is CR¹², X⁴ is CR¹⁴ and X³ is N.
Embodiment 14. A compound according to embodiments 13, wherein R¹¹, R¹² and R¹⁴ are independently selected from hydrogen, halogen, C₁₋₄alkyl, C₁₋₄alkoxy, hydroxy, cyano, —NR′R″,—CO₂R′, tetrazolyl and —SO₂—C₁₋₄alkyl, wherein said C₁₋₄alkyl is optionally substituted one or more times with a substituent independently selected from halogen, hydroxy and C1alkoxy and wherein R′ and R″ are each independently selected from hydrogen and C₁₋₄alkyl.
Embodiment 15. The compound according to embodiment 14, wherein R¹¹, R¹² and R¹⁴ is selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.
Embodiment 16. The compound according to anyone of embodiments 1-9, wherein X¹ is N; X² is selected from CR¹², X³ is N and X⁴ is selected from CR¹⁴.
Embodiment 17. A compound according to embodiment 16, wherein R¹² and R¹⁴ are independently selected from hydrogen, halogen, C₁₋₄alkyl, C₁₋₄alkoxy, hydroxy, cyano, —NR′R″, —CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.
Embodiment 18. The compound according to embodiment 17, wherein R¹² and R¹⁴ are independently selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.
Embodiment 19. The compound according to anyone of embodiments 1-9, wherein X¹ is N, X² is N; X³ is CR¹³ and X⁴ is CR¹⁴.
Embodiment 20. A compound according to embodiment 19, wherein R¹³ and R¹⁴ are independently selected from hydrogen, halogen, C₁₋₄alkyl, C₁₋₄alkoxy, hydroxy, cyano, —NR′R″, —CO₂R′, tetrazolyl and —SO₂—C-14alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.
Embodiment 21. The compound according to embodiment 20, wherein R¹³ and R¹⁴ are independently selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.
Embodiment 22. The compound according to any one of embodiments 1-9, wherein X¹ is CR¹¹, X² is N; X³ is N and X⁴ is CR¹⁴.
Embodiment 23. A compound according to embodiment 22, wherein R¹¹ and R¹⁴ are independently selected from hydrogen, halogen, C₁₋₄alkyl, C₁₋₄alkoxy, hydroxy, cyano, —NR′R″, —CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.
Embodiment 24. The compound according to embodiment 23, wherein R¹¹ and R¹⁴ are independently selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.
Embodiment 25. The compound according to any one of embodiments 1-9, wherein X¹, X⁴, X³ is N and X² is CR¹².
Embodiment 26. A compound according to embodiment 25, wherein R¹² is selected from hydrogen, halogen, C₁₋₄alkyl, C₁₋₄alkoxy, hydroxy, cyano, —NR′R″, —CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.
Embodiment 27. The compound according to embodiment 26, wherein R¹² is selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.
Embodiment 28. The compound according to anyone of embodiments 1-9, wherein X¹, X² is N, X³ is CR¹³ and X⁴ is CR¹⁴.
Embodiment 29. A compound according to embodiment 28, wherein R¹³ and R¹⁴ are independently selected from hydrogen, halogen, C₁₋₄alkyl, C₁₋₄alkoxy, hydroxy, cyano, —NR′R″, —CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.
Embodiment 30. The compound according to embodiment 29, wherein R¹³ and R¹⁴ are independently selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.
Embodiment 31. The compound according to any one of embodiments 1-9, wherein X³ and X⁴ is N and X¹ is CR¹¹ and X⁴ is CR¹⁴.
Embodiment 32. A compound according to embodiment 31, wherein R¹¹ and R¹⁴ are independently selected from hydrogen, halogen, C₁₋₄alkyl, C₁₋₄alkoxy, hydroxy, cyano, —NR′R″, —CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C1alkyl.
Embodiment 33. The compound according to embodiment 32, wherein R¹¹ and R¹⁴ are independently selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.
Embodiment 34. The compound according to any one of embodiments 1-9, wherein X¹ is CR¹¹, X² is CR¹², X³ is CR¹³, X⁴ is N⁺—O⁻.
Embodiment 35. A compound according to embodiment 34, wherein R¹¹, R¹² and R¹³ are independently selected from hydrogen, halogen, C₁₋₄alkyl, C₁₋₄alkoxy, hydroxy, cyano, —NR′R″,—CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.
Embodiment 36. The compound according to embodiment 35, wherein R¹¹, R¹² and R¹³ are independently selected from hydrogen, methyl, methoxy, hydroxy, hydroxymethyl, fluoro, chloro, di-or trifluoromethyl, cyano, methoxycarbonyl, —COOH, ethoxycarbonyl, methylsulfonyl, tetrazolyl and (CH₃)₂N—.
Embodiment 37. The compound according to any one of embodiments 1-36, wherein Z is N.
Embodiment 38. The compound according to any one of embodiments 1-36, wherein Z is CR¹⁰, and R⁵ and R¹⁰ together form a bond.
Embodiment 39. The compound according to any one of embodiments 1-36, wherein Z is CR¹⁰ and R¹⁰ is hydrogen.
Embodiment 40. The compound according to any one of embodiments 1-39, wherein R^{5a} is hydrogen.
Embodiment 41. The compound according to any one of embodiments 1-40, wherein Y¹ is CR⁷ and Y² is CR⁸.
Embodiment 42. The compound according to any one of embodiments 1-40, wherein Y¹ is CR⁷ and Y² is N.
Embodiment 43. The compound according to any one of embodiments 1-40, wherein Y¹ and Y² is N.
Embodiment 44. The compound according to embodiment 41, wherein
(a) both of R⁷ and R⁸ is C₁₋₄alkyl;
(b) both of R⁷ and R⁸ is halogen;
(c) one of R⁷ and R⁸ is C₁₋₄alkyl and the other is halogen;
(d) one of R⁷ and R⁸ is C₁₋₄alkyl and the other is hydrogen; or
(e) one of R⁷ and R⁸ is halogen and the other is hydrogen.
Embodiment 45. A compound according to embodiment 44, wherein one or both of R⁷ and R⁸ is hydrogen.
Embodiment 46. A compound according to embodiment 44, wherein one of R⁷ and R⁸ is hydrogen and the other is fluoro.
Embodiment 47. The compound according to any one of embodiments 1-46, wherein R is selected from-NH₂ and —NHCH₃
Embodiment 48. A compound according to any one of embodiments 1-47 wherein R¹ is hydrogen, R² and R³ is methyl.
Embodiment 48a. A compound selected from 3-((4-(1-(2-(4-(4-(1-((4-(1H-indazol-5-yl)-1-methyl-1H-pyrrolo[2,3-b]pyridin-2-yl)methyl)piperidin-4-yl)benzoyl) piperazin-1-yl)-2-oxoethyl)piperidin-4-yl)-3-fluorophenyl)amino)piperidine-2,6-dione;
3-((4-(4-(2-(4-(4-(1-((4-(1H-indazol-5-yl)-1-methyl-1H-pyrrolo[2,3-b]pyridin-2-yl)methyl)-piperidin-4-yl)benzoyl) piperazin-1-yl)-2-oxoethyl) piperazin-1-yl)-3-fluorophenyl)amino)piperidine-2,6-dione;
3-((4-(4-((1-(4-(1-((4-(1H-indazol-5-yl)-1-methyl-1H-pyrrolo[2,3-b]pyridin-2-yl)methyl)piperidin-4-yl)benzoyl)piperidin-4-yl)methyl)piperazin-1-yl)phenyl)amino)piperidine-2,6-dione; 3-((4-(4-((1-(4-(1-((4-(1H-indazol-5-yl)-1-methyl-1H-pyrrolo[2,3-b]pyridin-2-yl)methyl)piperidin-4-yl)benzoyl)piperidin-4-yl)methyl)piperazin-1-yl)-2-fluorophenyl)amino)piperidine-2,6-dione;
3-((2-fluoro-4-(4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperazin-1-yl)phenyl)amino)piperidine-2,6-dione;
3-((4-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)-3-fluorophenyl)amino)piperidine-2,6-dione;
3-((4-(4-((1-(4-(1-((4-(6-aminopyridin-3-yl)-1-methyl-1H-pyrrolo[2,3-b]pyridin-2-yl)methyl)-piperidin-4-yl)benzoyl)piperidin-4-yl)methyl)piperazin-1-yl)-3-fluorophenyl)amino)piperidine-2,6-dione;
3-((4-(4-((1-(4-(1-((4-(6-amino-5-fluoropyridin-3-yl)-1-methyl-1H-pyrrolo[2,3-b]pyridin-2-yl)-methyl)piperidin-4-yl)benzoyl)piperidin-4-yl)methyl)piperazin-1-yl)-3-fluorophenyl)amino)piperidine-2,6-dione;
3-((3-fluoro-4-(4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperazin-1-yl)phenyl)-amino)piperidine-2,6-dione;
(R)-3-((4-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)-3-fluorophenyl)amino)piperidine-2,6-dione;
(S)-3-((4-(4-((1-(4-(1-((4-(1H-indazol-5-yl)-1-methyl-1H-pyrrolo[2,3-b]pyridin-2-yl)methyl)piperidin-4-yl)benzoyl)piperidin-4-yl)-methyl)piperazin-1-yl)-3-fluorophenyl)amino)piperidine-2,6-dione;
3-((4-(4-((1-(4-(1-((4-(6-amino-2-methylpyridin-3-yl)-1-methyl-1H-pyrrolo[2,3-b]pyridin-2-yl)methyl)piperidin-4-yl)benzoyl)piperidin-4-yl)methyl)piperazin-1-yl)-3-fluorophenyl)amino)piperidine-2,6-dione;
6-amino-3-(2-((4-(4-(4-((4-(4-((2,6-dioxopiperidin-3-yl)amino)-2-fluorophenyl) piperazin-1-yl)-methyl)piperidine-1-carbonyl)phenyl) piperidin-1-yl)methyl)-1-methyl-1H-pyrrolo[2,3-b]pyridin-4-yl)-picolinonitrile;
3-((4-(4-((1-(4-(1-((4-(6-aminopyridin-3-yl)-1-methyl-1H-pyrrolo[2,3-b]pyridin-2-yl)methyl)-1,2,3,6-tetrahydropyridin-4-yl)-3-methylbenzoyl)piperidin-4-yl)methyl)piperazin-1-yl)-3-fluorophenyl)-amino)piperidine-2,6-dione;
3-((4-(4-((1-(4-(1-(1-(4-(6-aminopyridin-3-yl)-1-methyl-1H-pyrrolo[2,3-b]pyridin-2-yl)-ethyl)-piperidin-4-yl)benzoyl)piperidin-4-yl)methyl)piperazin-1-yl)-3-fluorophenyl)amino)piperidine-2,6-dione;
(3R)-3-((4-(4-((1-(4-(1-(1-(4-(6-aminopyridin-3-yl)-1-methyl-1H-pyrrolo[2,3-b]pyridin-2-yl)ethyl)piperidin-4-yl)benzoyl)piperidin-4-yl)methyl)piperazin-1-yl)-3-fluorophenyl)amino)piperidine-2,6-dione;
(3S)-3-((4-(4-((1-(4-(1-(1-(4-(6-aminopyridin-3-yl)-1-methyl-1H-pyrrolo[2,3-b]pyridin-2-yl)-ethyl)piperidin-4-yl)benzoyl)piperidin-4-yl)methyl)piperazin-1-yl)-3-fluorophenyl)amino)piperidine-2,6-dione;
3-((4-(4-((1-(4-(1-((R)-1-(4-(6-aminopyridin-3-yl)-1-methyl-1H-pyrrolo[2,3-b]pyridin-2-yl)ethyl)-piperidin-4-yl)benzoyl)piperidin-4-yl)methyl)piperazin-1-yl)-3-fluorophenyl)amino)piperidine-2,6-dione;
3-((4-(4-((1-(4-(1-((S)-1-(4-(6-aminopyridin-3-yl)-1-methyl-1H-pyrrolo[2,3-b]pyridin-2-yl)ethyl)-piperidin-4-yl)benzoyl)piperidin-4-yl)methyl)piperazin-1-yl)-3-fluorophenyl)amino)piperidine-2,6-dione;
3-((4-(4-((1-(4-(1-((4-(6-aminopyridin-3-yl)-1-methyl-1H-pyrrolo[2,3-b]pyridin-2-yl)methyl)-1,2,3,6-tetrahydropyridin-4-yl)-3,5-dimethylbenzoyl)piperidin-4-yl)methyl)piperazin-1-yl)-3-fluorophenyl)amino)piperidine-2,6-dione;
3-((3-fluoro-4-(4-((1-(4-(1-((S)-1-(4-(2-(methoxymethyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione;
3-((4-(4-((1-(4-(1-((S)-1-(4-(1H-indazol-5-yl)-1-methyl-1H-pyrrolo[2,3-b]pyridin-2-yl)-ethyl)piperidin-4-yl)benzoyl)piperidin-4-yl)methyl)piperazin-1-yl)-3-fluorophenyl)amino)piperidine-2,6-dione;
3-((3-fluoro-4-(4-((1-(4-(1-((S)-1-(4-(2-((S)-1-methoxyethyl)-1H-benzo[d]imidazol-5-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione;
(S)-3-((4-(4-((1-(4-(1-((S)-1-(4-(6-aminopyridin-3-yl)-1-methyl-1H-pyrrolo[2,3-b]pyridin-2-yl)ethyl)piperidin-4-yl)benzoyl)piperidin-4-yl)methyl)piperazin-1-yl)-3-fluorophenyl)-amino)-piperidine-2,6-dione;
(R)-3-((4-(4-((1-(4-(1-((S)-1-(4-(6-aminopyridin-3-yl)-1-methyl-1H-pyrrolo[2,3-b]pyridin-2-yl)ethyl)-piperidin-4-yl)benzoyl)piperidin-4-yl)methyl)piperazin-1-yl)-3-fluorophenyl)amino)-piperidine-2,6-dione;
3-((3-fluoro-4-(4-((1-(4-(1-((S)-1-(4-(2-((R)-1-methoxyethyl)-1H-benzo[d]imidazol-5-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione;
6-amino-3-(2-((1S)-1-(4-(4-(4-((4-(4-((2,6-dioxopiperidin-3-yl)amino)-2-fluorophenyl)-piperazin-1-yl)methyl)piperidine-1-carbonyl)phenyl) piperidin-1-yl)ethyl)-1-methyl-1H-pyrrolo[2,3-b]pyridin-4-yl) picolinonitrile;
3-((4-(4-((1-(4-(1-((S)-1-(4-(6-aminopyridin-3-yl)-1-methyl-1H-pyrrolo[2,3-b]pyridin-2-yl)ethyl)-piperidin-4-yl)benzoyl)-4-fluoropiperidin-4-yl)methyl)piperazin-1-yl)-3-fluorophenyl)amino)-piperidine-2,6-dione;
3-((4-(4-((1-(4-(1-((S)-1-(4-(6-amino-5-fluoropyridin-3-yl)-1-methyl-1H-pyrrolo[2,3-b]pyridin-2-yl)ethyl)piperidin-4-yl)benzoyl)piperidin-4-yl)methyl)piperazin-1-yl)-3-fluorophenyl)-amino)piperidine-2,6-dione;
3-((3-fluoro-4-(4-((1-(4-(1-((S)-1-(4-(6-((2-hydroxyethyl)amino)pyridin-3-yl)-1-methyl-1H-pyrrolo[2,3-b]pyridin-2-yl)ethyl)piperidin-4-yl)benzoyl)piperidin-4-yl)methyl)piperazin-1-yl)phenyl)-amino)piperidine-2,6-dione;
3-((4-(4-((1-(4-(1-((S)-1-(4-(6-aminopyridazin-3-yl)-1-methyl-1H-pyrrolo[2,3-b]pyridin-2-yl)ethyl)piperidin-4-yl)benzoyl)piperidin-4-yl)methyl)piperazin-1-yl)-3-fluorophenyl)amino)piperidine-2,6-dione;
3-((3-fluoro-4-(4-((1-(4-(1-((S)-1-(1-methyl-4-(3-methyl-2-oxo-2,3-dihydro-1H-benzo[d]imidazol-5-yl)-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione;
3-((4-(4-((1-(4-(1-((S)-1-(4-(5-amino-3-fluoropyridin-2-yl)-1-methyl-1H-pyrrolo[2,3-b]pyridin-2-yl)ethyl)piperidin-4-yl)benzoyl)piperidin-4-yl)methyl)piperazin-1-yl)-3-fluorophenyl)-amino)piperidine-2,6-dione;
3-((4-(4-((1-(4-(1-((S)-1-(4-(6-amino-2-methoxypyridin-3-yl)-1-methyl-1H-pyrrolo[2,3-b]pyridin-2-yl)ethyl)piperidin-4-yl)benzoyl)piperidin-4-yl)methyl)piperazin-1-yl)-3-fluorophenyl)amino)-piperidine-2,6-dione;
3-((4-(4-((1-(4-(1-((S)-1-(4-(5-aminopyridin-2-yl)-1-methyl-1H-pyrrolo[2,3-b]pyridin-2-yl)ethyl)-piperidin-4-yl)benzoyl)piperidin-4-yl)methyl)piperazin-1-yl)-3-fluorophenyl)amino)piperidine-2,6-dione;
3-((4-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)-3,5-difluorophenyl)amino)piperidine-2,6-dione;
6-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)-N-(2,6-dioxopiperidin-3-yl) pyridazine-3-carboxamide;
3-((4-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazine-1-carbonyl)-3-fluorophenyl)amino)piperidine-2,6-dione;
3-((4-(4-((1-(4-(1-((4-(6-aminopyridin-3-yl)-1-methyl-1H-pyrrolo[2,3-b]pyridin-2-yl)methyl)-piperidin-4-yl)benzoyl)piperidin-4-yl)methyl)piperazine-1-carbonyl)-3-fluorophenyl)amino)piperidine-2,6-dione;
3-((3-fluoro-4-((R)-4-((1-(4-(1-((S)-1-(4-(2-(methoxymethyl)-1H-benzo[d]imidazol-5-yl)-1-methyl-1H-pyrrolo[2,3-b]pyridin-2-yl)ethyl)piperidin-4-yl)benzoyl)piperidin-4-yl)methyl)-3-methylpiperazin-1-yl)phenyl)amino)piperidine-2,6-dione;
3-((4-((R)-4-((1-(4-(1-((S)-1-(4-(6-aminopyridin-3-yl)-1-methyl-1H-pyrrolo[2,3-b]pyridin-2-yl)ethyl)piperidin-4-yl)benzoyl)piperidin-4-yl)methyl)-3-methylpiperazin-1-yl)-3-fluorophenyl)amino)-piperidine-2,6-dione;
3-((4-((S)-4-((1-(4-(1-((4-(6-aminopyridin-3-yl)-1-methyl-1H-pyrrolo[2,3-b]pyridin-2-yl)methyl)piperidin-4-yl)benzoyl)piperidin-4-yl)methyl)-3-methylpiperazin-1-yl)-3-fluorophenyl)amino)-piperidine-2,6-dione;
3-((3-fluoro-4-((S)-4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)-3-methylpiperazin-1-yl)phenyl)-amino)piperidine-2,6-dione:
3-((4-((S)-4-((1-(4-(1-((4-(1H-indazol-5-yl)-1-methyl-1H-pyrrolo[2,3-b]pyridin-2-yl)methyl)piperidin-4-yl)benzoyl)piperidin-4-yl)methyl)-3-methylpiperazin-1-yl)-3-fluorophenyl)amino)piperidine-2,6-dione;
3-((4-((R)-4-((1-(4-(1-((4-(6-aminopyridin-3-yl)-1-methyl-1H-pyrrolo[2,3-b]pyridin-2-yl)-methyl)-piperidin-4-yl)benzoyl)piperidin-4-yl)methyl)-3-methylpiperazin-1-yl)-3-fluorophenyl)amino)-piperidine-2,6-dione;
3-((3-fluoro-4-((R)-4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)-3-methylpiperazin-1-yl)phenyl)-amino)piperidine-2,6-dione;
3-((4-((R)-4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)-3-methylpiperazin-1-yl)-3-fluorophenyl)amino)piperidine-2,6-dione;
3-((4-(4-((1-(4-(1-((S)-1-(4-(6-aminopyridin-3-yl)-1-methyl-1H-pyrrolo[2,3-b]pyridin-2-yl)-ethyl)piperidin-4-yl)benzoyl)piperidin-4-yl)methyl)-3-(trifluoromethyl)piperazin-1-yl)-3-fluorophenyl)-amino)piperidine-2,6-dione;
3-((4-((S)-4-((1-(4-(1-((S)-1-(4-(6-aminopyridin-3-yl)-1-methyl-1H-pyrrolo[2,3-b]pyridin-2-yl)ethyl)piperidin-4-yl)benzoyl)piperidin-4-yl)methyl)-3-(trifluoromethyl)piperazin-1-yl)-3-fluorophenyl)-amino)piperidine-2,6-dione;
3-((4-((R)-4-((1-(4-(1-((S)-1-(4-(6-aminopyridin-3-yl)-1-methyl-1H-pyrrolo[2,3-b]pyridin-2-yl)ethyl)piperidin-4-yl)benzoyl)piperidin-4-yl)methyl)-3-(trifluoromethyl)piperazin-1-yl)-3-fluoro-phenyl)amino)piperidine-2,6-dione;
(S)-3-((3-fluoro-4-((R)-4-((1-(4-(1-((S)-1-(4-(2-(methoxymethyl)-1H-benzo[d]-imidazol-5-yl)-1-methyl-1H-pyrrolo[2,3-b]pyridin-2-yl)ethyl)piperidin-4-yl)benzoyl)piperidin-4-yl)-methyl)-3-methylpiperazin-1-yl)phenyl)amino)piperidine-2,6-dione;
(R)-3-((3-fluoro-4-((R)-4-((1-(4-(1-((S)-1-(4-(2-(methoxymethyl)-1H-benzo[d]imidazol-5-yl)-1-methyl-1H-pyrrolo[2,3-b]pyridin-2-yl)-ethyl)piperidin-4-yl)benzoyl)piperidin-4-yl)methyl)-3-methylpiperazin-1-yl)phenyl)amino)piperidine-2,6-dione;
3-((1-(1-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperidin-4-yl)-1H-indazol-4-yl)amino)piperidine-2,6-dione;
3-((4-(1′-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)-3′, 3′-difluoro-[1,4′-bipiperidin]-4-yl)-3-fluorophenyl)-amino)piperidine-2,6-dione;
3-((4-(1′-((1-(4-(1-((4-(6-aminopyridin-3-yl)-1-methyl-1H-pyrrolo[2,3-b]pyridin-2-yl)-methyl)piperidin-4-yl)benzoyl)piperidin-4-yl)methyl)-3′, 3′-difluoro-[1,4′-bipiperidin]-4-yl)-3-fluoro-phenyl)amino)piperidine-2,6-dione
3-((4-(3′, 3′-difluoro-1′-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)-[1,4′-bipiperidin]-4-yl)-3-fluorophenyl)amino)piperidine-2,6-dione;
3-((4-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)-5-fluoro-2-methoxyphenyl)amino)-piperidine-2,6-dione;
5-((2,6-dioxopiperidin-3-yl)amino)-2-(4-((1-(4-(1-((S)-1-(4-(2-(methoxymethyl)-1H-benzo[d]-imidazol-5-yl)-1-methyl-1H-pyrrolo[2,3-b]pyridin-2-yl)ethyl)piperidin-4-yl)benzoyl)piperidin-4-yl)methyl)-piperazin-1-yl)benzonitrile;
2-(4-((1-(4-(1-((S)-1-(4-(6-aminopyridin-3-yl)-1-methyl-1H-pyrrolo[2,3-b]pyridin-2-yl)-ethyl)-piperidin-4-yl)benzoyl)piperidin-4-yl)methyl)piperazin-1-yl)-5-((2,6-dioxopiperidin-3-yl)amino)-benzonitrile;
3-((3-chloro-4-(4-((1-(4-(1-((S)-1-(4-(2-(methoxymethyl)-1H-benzo[d]imidazol-5-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione;
3-((4-(4-((1-(4-(1-((S)-1-(4-(6-aminopyridin-3-yl)-1-methyl-1H-pyrrolo[2,3-b]pyridin-2-yl)-ethyl)piperidin-4-yl)benzoyl)piperidin-4-yl)methyl)piperazin-1-yl)-3-chlorophenyl)amino)piperidine-2,6-dione;
3-((4-(4-((1-(4-(1-((S)-1-(4-(6-aminopyridin-3-yl)-1-methyl-1H-pyrrolo[2,3-b]pyridin-2-yl)-ethyl)-piperidin-4-yl)benzoyl)piperidin-4-yl)methyl)piperazin-1-yl)-3-(trifluoromethyl)phenyl)amino)-piperidine-2,6-dione;
4-(1-((4-(1H-indazol-5-yl)-1-methyl-1H-pyrrolo[2,3-b]pyridin-2-yl)methyl)piperidin-4-yl)-N-(4-(1-(4-((2,6-dioxopiperidin-3-yl)amino)-2-fluorophenyl) piperidin-4-yl)phenyl)-N-methylbenzamide;
4-(1-((4-(1H-indazol-5-yl)-1-methyl-1H-pyrrolo[2,3-b]pyridin-2-yl)methyl)piperidin-4-yl)-N-(4-(1-(4-((2,6-dioxopiperidin-3-yl)amino)phenyl) piperidin-4-yl)phenyl)-N-methylbenzamide; 3-((2-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)pyrimidin-5-yl)amino)piperidine-2,6-dione;
3-((2-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)pyrimidin-4-yl)amino)piperidine-2,6-dione;
3-((4-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)pyrimidin-2-yl)amino)piperidine-2,6-dione;
3-((3-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)phenyl)amino)piperidine-2,6-dione;
3-((4-(1-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperidin-4-yl)-3-fluorophenyl)amino)piperidine-2,6-dione;
3-((4-(1-((1-(4-(1-((S)-1-(4-(6-aminopyridin-3-yl)-1-methyl-1H-pyrrolo[2,3-b]pyridin-2-yl)-ethyl)piperidin-4-yl)benzoyl)piperidin-4-yl)methyl)-3,3-difluoropiperidin-4-yl)-3-fluorophenyl)amino)-piperidine-2,6-dione;
3-((4-(3,3-difluoro-1-((1-(4-(1-((S)-1-(4-(2-(methoxymethyl)-1H-benzo[d]imidazol-5-yl)-1-methyl-1H-pyrrolo[2,3-b]pyridin-2-yl)ethyl)piperidin-4-yl)benzoyl)piperidin-4-yl)methyl)piperidin-4-yl)-3-fluorophenyl)amino)piperidine-2,6-dione;
3-((6-(4-((1-(4-(1-((4-(1H-indazol-5-yl)-1-methyl-1H-pyrrolo[2,3-b]pyridin-2-yl)methyl)piperidin-4-yl)benzoyl)piperidin-4-yl)methyl)piperazin-1-yl)pyridin-3-yl)amino)piperidine-2,6-dione;
3-((6-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)-2-fluoropyridin-3-yl)amino)piperidine-2,6-dione;
3-((2-fluoro-6-(4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperazin-1-yl)pyridin-3-yl)amino)piperidine-2,6-dione;
3-((6-(4-((1-(4-(1-((4-(6-aminopyridin-3-yl)-1-methyl-1H-pyrrolo[2,3-b]pyridin-2-yl)methyl)-piperidin-4-yl)benzoyl)piperidin-4-yl)methyl)piperazin-1-yl)-2-fluoropyridin-3-yl)amino)piperidine-2,6-dione;
3-((6-(4-((1-(4-(1-((4-(6-aminopyridin-3-yl)-1-methyl-1H-pyrrolo[2,3-b]pyridin-2-yl)methyl)-piperidin-4-yl)benzoyl)piperidin-4-yl)methyl)piperazin-1-yl)-5-fluoropyridin-3-yl)amino)piperidine-2,6-dione;
3-((5-fluoro-6-(4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperazin-1-yl)pyridin-3-yl)amino)piperidine-2,6-dione;
3-((6-(4-((1-(4-(1-((S)-1-(4-(1H-indazol-5-yl)-1-methyl-1H-pyrrolo[2,3-b]pyridin-2-yl)ethyl)-piperidin-4-yl)benzoyl)piperidin-4-yl)methyl)piperazin-1-yl)-2-fluoropyridin-3-yl)amino)piperidine-2,6-dione;
3-((6-(4-((1-(4-(1-((S)-1-(4-(1H-indazol-5-yl)-1-methyl-1H-pyrrolo[2,3-b]pyridin-2-yl)ethyl)-piperidin-4-yl)benzoyl)piperidin-4-yl)methyl)piperazin-1-yl)-5-fluoropyridin-3-yl)amino)piperidine-2,6-dione;
3-((6-(4-((1-(4-(1-((S)-1-(4-(6-aminopyridin-3-yl)-1-methyl-1H-pyrrolo[2,3-b]pyridin-2-yl)-ethyl)-piperidin-4-yl)benzoyl)piperidin-4-yl)methyl)piperazin-1-yl)-2-fluoropyridin-3-yl)amino)piperidine-2,6-dione;
3-((6-(4-((1-(4-(1-((S)-1-(4-(6-aminopyridin-3-yl)-1-methyl-1H-pyrrolo[2,3-b]pyridin-2-yl)ethyl)-piperidin-4-yl)benzoyl)piperidin-4-yl)methyl)piperazin-1-yl)-5-fluoropyridin-3-yl)amino)-piperidine-2,6-dione;
3-((2-fluoro-6-(4-((1-(4-(1-((S)-1-(4-(2-(methoxymethyl)-1H-benzo[d]imidazol-5-yl)-1-methyl-1H-pyrrolo[2,3-b]pyridin-2-yl)ethyl)piperidin-4-yl)benzoyl)piperidin-4-yl)methyl)piperazin-1-yl)pyridin-3-yl)amino)piperidine-2,6-dione;
3-((5-fluoro-6-(4-((1-(4-(1-((S)-1-(4-(2-(methoxymethyl)-1H-benzo[d]imidazol-5-yl)-1-methyl-1H-pyrrolo[2,3-b]pyridin-2-yl)ethyl)piperidin-4-yl)benzoyl)piperidin-4-yl)methyl)piperazin-1-yl)pyridin-3-yl)amino)piperidine-2,6-dione;
3-((5-fluoro-6-(4-((1-(4-(1-((S)-1-(1-methyl-4-(6-(methylamino)pyridin-3-yl)-1H-pyrrolo[2,3-b] pyridin-2-yl)ethyl)piperidin-4-yl)benzoyl)piperidin-4-yl)methyl)piperazin-1-yl)pyridin-3-yl)amino)piperidine-2,6-dione;
3-((2-fluoro-6-(4-((1-(4-(1-((S)-1-(1-methyl-4-(6-(methylamino)pyridin-3-yl)-1H-pyrrolo[2,3-b] pyridin-2-yl)ethyl)piperidin-4-yl)benzoyl)piperidin-4-yl)methyl)piperazin-1-yl)pyridin-3-yl)amino)piperidine-2,6-dione;
4-(1-((4-(6-aminopyridin-3-yl)-1-methyl-1H-pyrrolo[2,3-b]pyridin-2-yl)methyl)piperidin-4-yl)-N-(4-((1-(4-((2,6-dioxopiperidin-3-yl)amino)phenyl) piperidin-4-yl)methyl) phenyl)-N-methylbenzamide;
4-(1-((4-(1H-indazol-5-yl)-1-methyl-1H-pyrrolo[2,3-b]pyridin-2-yl)methyl)piperidin-4-yl)-N-(4-((1-(4-((2,6-dioxopiperidin-3-yl)amino)phenyl) piperidin-4-yl)methyl) phenyl)-N-methylbenzamide;
3-((4-(4-(1-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)-3,3-difluoropiperidin-4-yl) piperazin-1-yl)-3-fluorophenyl)-amino)piperidine-2,6-dione;
3-((4-(4-(1-((1-(4-(1-((4-(6-aminopyridin-3-yl)-1-methyl-1H-pyrrolo[2,3-b]pyridin-2-yl)-methyl)piperidin-4-yl)benzoyl)piperidin-4-yl)methyl)-3,3-difluoropiperidin-4-yl) piperazin-1-yl)-3-fluorophenyl)amino)piperidine-2,6-dione;
3-((4-(4-(3,3-difluoro-1-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperidin-4-yl) piperazin-1-yl)-3-fluorophenyl)amino)piperidine-2,6-dione;
3-((4-(1-((7-(4-(1-((4-(6-aminopyridin-3-yl)-1-methyl-1H-pyrrolo[2,3-b]pyridin-2-yl)methyl)piperidin-4-yl)benzoyl)-5,6,7,8-tetrahydro-[1,2,4]triazolo[4,3-a]pyrazin-3-yl)methyl)piperidin-4-yl)-3-fluorophenyl)amino)piperidine-2,6-dione;
3-((4-(1-((7-(4-(1-((4-(1H-indazol-5-yl)-1-methyl-1H-pyrrolo[2,3-b]pyridin-2-yl)methyl)-piperidin-4-yl)benzoyl)-5,6,7,8-tetrahydro-[1,2,4]triazolo[4,3-a]pyrazin-3-yl)methyl)piperidin-4-yl)-3-fluorophenyl)amino)piperidine-2,6-dione;
3-((4-(1-((5-(4-(1-((4-(1H-indazol-5-yl)-1-methyl-1H-pyrrolo[2,3-b]pyridin-2-yl)methyl)-piperidin-4-yl)benzoyl)-4,5,6,7-tetrahydropyrazolo[1,5-a]pyrazin-2-yl)methyl)piperidin-4-yl)-3-fluorophenyl)amino)piperidine-2,6-dione;
3-((4-(1-((5-(4-(1-((4-(6-aminopyridin-3-yl)-1-methyl-1H-pyrrolo[2,3-b]pyridin-2-yl)methyl)-piperidin-4-yl)benzoyl)-4,5,6,7-tetrahydropyrazolo[1,5-a]pyrazin-2-yl)methyl)piperidin-4-yl)-3-fluoro-phenyl)amino)piperidine-2,6-dione;
3-((4-(1-((5-(4-(1-((S)-1-(4-(6-aminopyridin-3-yl)-1-methyl-1H-pyrrolo[2,3-b]pyridin-2-yl)ethyl)-piperidin-4-yl)benzoyl)-4,5,6,7-tetrahydropyrazolo[1,5-a]pyrazin-2-yl)methyl)piperidin-4-yl)-3-fluorophenyl)-amino)piperidine-2,6-dione;
3-((6-(1-((5-(4-(1-((S)-1-(4-(6-aminopyridin-3-yl)-1-methyl-1H-pyrrolo[2,3-b]pyridin-2-yl)ethyl)-piperidin-4-yl)benzoyl)-4,5,6,7-tetrahydropyrazolo[1,5-a]pyrazin-2-yl)methyl)piperidin-4-yl)-5-fluoropyridin-3-yl)amino)piperidine-2,6-dione;
3-((3-fluoro-4-(1-((5-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)-4,5,6,7-tetrahydropyrazolo[1,5-a]pyrazin-2-yl)-methyl)piperidin-4-yl)phenyl)amino)piperidine-2,6-dione;
3-((5-fluoro-6-(1-((5-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)-4,5,6,7-tetrahydropyrazolo[1,5-a]pyrazin-2-yl)methyl)piperidin-4-yl)pyridin-3-yl)amino)piperidine-2,6-dione;
3-((4-(1-((5-(4-(1-((S)-1-(4-(6-aminopyridin-3-yl)-1-methyl-1H-pyrrolo[2,3-b]pyridin-2-yl)-ethyl)piperidin-4-yl)benzoyl)-4,5,6,7-tetrahydropyrazolo[1,5-a]pyrazin-2-yl)methyl)-3,3-difluoro-piperidin-4-yl)-3-fluorophenyl)amino)piperidine-2,6-dione;
(R)-3-((4-(1-((5-(4-(1-((S)-1-(4-(6-aminopyridin-3-yl)-1-methyl-1H-pyrrolo[2,3-b]pyridin-2-yl)ethyl)piperidin-4-yl)benzoyl)-4,5,6,7-tetrahydropyrazolo[1,5-a]pyrazin-2-yl)methyl)piperidin-4-yl)-3-fluorophenyl)amino)piperidine-2,6-dione;
(S)-3-((4-(1-((5-(4-(1-((S)-1-(4-(6-aminopyridin-3-yl)-1-methyl-1H-pyrrolo[2,3-b]pyridin-2-yl)ethyl)piperidin-4-yl)benzoyl)-4,5,6,7-tetrahydropyrazolo[1,5-a]pyrazin-2-yl)methyl)piperidin-4-yl)-3-fluorophenyl)amino)piperidine-2,6-dione; 3-((4-(3,3-difluoro-1-((5-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)-4,5,6,7-tetrahydropyrazolo[1,5-a]pyrazin-2-yl)methyl)piperidin-4-yl)-3-fluorophenyl)amino)piperidine-2,6-dione; 3-((6-(4-((5-(4-(1-((S)-1-(4-(6-aminopyridin-3-yl)-1-methyl-1H-pyrrolo[2,3-b]pyridin-2-yl)ethyl)-piperidin-4-yl)benzoyl)-4,5,6,7-tetrahydropyrazolo[1,5-a]pyrazin-2-yl)methyl)piperazin-1-yl)-5-fluoropyridin-3-yl)amino)piperidine-2,6-dione;
3-((4-(4-((5-(4-(1-((S)-1-(4-(6-aminopyridin-3-yl)-1-methyl-1H-pyrrolo[2,3-b]pyridin-2-yl)-ethyl)piperidin-4-yl)benzoyl)-4,5,6,7-tetrahydropyrazolo[1,5-a]pyrazin-2-yl)methyl)piperazin-1-yl)-3-fluoro-phenyl)amino)piperidine-2,6-dione;
3-((4-(4-((1-(3-(4-(1-((4-(6-aminopyridin-3-yl)-1-methyl-1H-pyrrolo[2,3-b]pyridin-2-yl)methyl)-piperidin-4-yl)phenyl)-[1,2,4]triazolo[4,3-b]pyridazin-6-yl) piperidin-4-yl)methyl)piperazin-1-yl)-3-fluorophenyl)-amino)piperidine-2,6-dione;
3-((4-((4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazin-1-yl)methyl) phenyl)amino)piperidine-2,6-dione;
3-((4-((4-((1-(4-(1-((4-(6-aminopyridin-3-yl)-1-methyl-1H-pyrrolo[2,3-b]pyridin-2-yl)methyl)piperidin-4-yl)benzoyl)piperidin-4-yl)methyl)piperazin-1-yl)methyl)-3-fluorophenyl)amino)piperidine-2,6-dione;
3-(4-(1-(2-(4-(4-(1-((4-(1H-indazol-5-yl)-1-methyl-1H-pyrrolo[2,3-b]pyridin-2-yl)methyl)-piperidin-4-yl)benzoyl) piperazin-1-yl)-2-oxoethyl)piperidin-4-yl)-3-fluorophenoxy) piperidine-2,6-dione;
3-((2-(4-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperazine-1-carbonyl)phenyl)amino)piperidine-2,6-dione;
3-((5-(4-((1-(4-(1-((4-(1H-indazol-5-yl)-1-methyl-1H-pyrrolo[2,3-b]pyridin-2-yl)methyl)piperidin-4-yl)benzoyl)piperidin-4-yl)methyl)piperazin-1-yl)pyridin-2-yl)amino)piperidine-2,6-dione;
3-((1-(1-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)methyl)piperidin-4-yl)-1H-indazol-5-yl)amino)piperidine-2,6-dione 3-((3-(3-((1-(4-(1-((4-(1H-indazol-5-yl)-1-methyl-1H-pyrrolo[2,3-b]pyridin-2-yl)methyl)-piperidin-4-yl)benzoyl)piperidin-4-yl)oxy) prop-1-yn-1-yl)phenyl)amino)piperidine-2,6-dione or pharmaceutically acceptable salts thereof.
   Embodiment 49. A compound according to any one of embodiments 1-48 for use in therapy.
   Embodiment 50. A compound according to embodiment 49 for use in the treatment of a disease, disorder or condition, which disease, disorder or condition is responsive of modulation of STAT-6.
   Embodiment 51. A compound according to embodiment 49 for use in the treatment of autoimmune diseases.
   Embodiment 52. A compound according to embodiment 49 for use in the treatment diseases characterized by Th2-mediated inflammation such as atopic dermatitis, asthma, chronic rhinosinusitis with nasal polyposis, urticaria, rhinitis, eosinophilic esophagitis, food allergy diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors.
   Embodiment 53. A pharmaceutical composition comprising a compound according to any one of embodiments 1-48 together with a pharmaceutically acceptable vehicle or excipient or pharmaceutically acceptable carrier(s).

1. A compound according to formula (I): 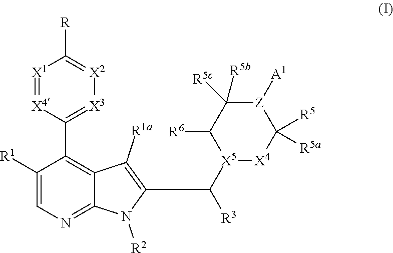
A′ is selected from 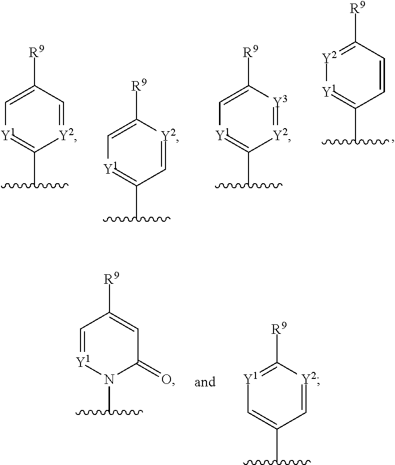
X¹ is selected from CR¹¹ and N; X² is selected from CR¹² and N; X³ is selected from CR¹³ and N, and X⁴ is selected from CR¹⁴ and N, and wherein when X⁴ is N the N may be an N-oxide, or X¹ is CR¹¹ and taken together with R forms a 5-membered heteroaryl ring which is optionally substituted with C (O) CF₃;
X⁵ is selected from CR⁴R⁴ and CO;
X^{a} is selected from N and oxidized N;
Y¹ is selected from N and CR⁷;
Y² is selected from N and CR⁸;
Y³ is selected from N and CR¹⁷;
Z is selected from N and CR¹⁰;
R is selected from NHR⁰ and —CONHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—R′, —(CH₂)ₙ—CO—C₁₋₄alkyl and —(CH₂)ₙ—CO₂R′, wherein said phenyl is optionally substituted one or more times with substituents independently selected from halogen, C₁₋₄alkyl, halo-C₁₋₄alkyl, C₃₋₄ cycloalkyl, hydroxy, C₁₋₄alkoxy, cyano, —NR′R″, —CONR′R″ and —CO₂R′, wherein R′ and R″ are each independently selected from hydrogen and C₁₋₄alkyl and n is 0, 1 or 2;
R¹ and R^{1a} are independently selected from hydrogen, fluoro and C₁₋₄alkyl;
R² is selected from hydrogen, C₁₋₅alkyl,—SO₂—C₁₋₄alkyl and deuterated C₁₋₄alkyl, wherein said C₁₋₅alkyl may optionally be substituted one or more times with fluoro;
R³ is independently selected from hydrogen, C₁₋₄alkyl, and deuterated C₁₋₄alkyl;
each R⁴ and R⁶ are independently selected from hydrogen, C₁₋₄alkyl, and C₁₋₄alkoxy, wherein said C₁₋₄alkyl and C₁₋₄alkoxy may optionally be substituted with one or more halogen;
R⁵ is selected from hydrogen, halogen, C₁₋₄alkyl and C₁₋₄alkoxy, wherein said C₁₋₄alkyl and C₁₋₄alkoxy may optionally be substituted with one or more halogen;
R¹⁰ is selected from hydrogen and fluoro; or
R⁵ and R¹⁰ together form a bond between the two carbon atoms to which they are attached;
R^{5a} is hydrogen;
or R⁵ and R^{5a} are both fluoro or R⁵ and R^{5a} together with the atom attached thereto form a CO group;
R^{5b} and R^{5c} are each independently selected from hydrogen and fluoro;
R⁷, R⁸, and R¹⁷ are each independently selected from hydrogen, halogen, cyano, C₁₋₄alkyl and C₁₋₄alkoxy, and C₃₋₄cycloalkyl, wherein said C₁₋₄alkyl and C₁₋₄alkoxy may optionally be substituted with one or more halogen;
R⁹ is-A-L-LBM;
R¹¹, R¹², R¹³ and R¹⁴ are each independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′, —CO—(CH₂)ₘOH, tetrazolyl and —SO₂—C₁₋₄alkyl, wherein said C₁₋₄alkyl and C₁₋₄alkoxy are optionally substituted one or more times with a substituent independently selected from halogen, hydroxy and C₁₋₄alkoxy, wherein m is 1, 2 or 3; or
R¹¹ and R⁰ together with the atoms attached thereto form a 5-6 membered heteroaromatic or heterocyclic ring containing one or two N atoms, wherein said 5-6 membered heteroaromatic ring or heterocyclic ring containing one or two N atoms is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —COF₃, —NR′R″, —CONR′R″ and —CO₂R′, wherein said C₁₋₄alkyl may optionally be substituted one or more times with a substituent independently selected from halogen and C₁₋₄alkoxy;
A is CO or 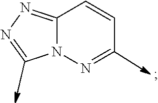
L is selected from: 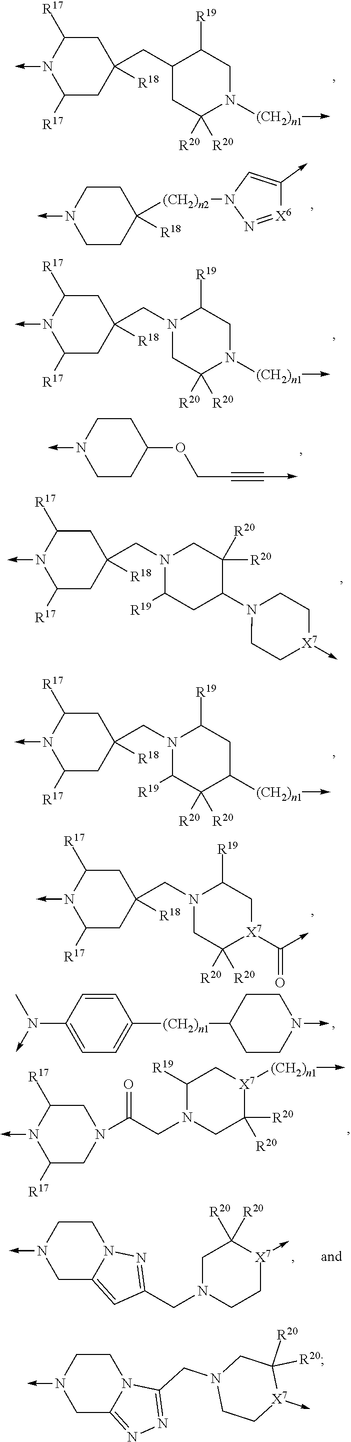
wherein
R¹⁷ is selected from hydrogen and fluoro;
R¹⁸ is selected from hydrogen and fluoro;
R¹⁹ is selected from hydrogen and C₁₋₄alkyl, wherein said C₁₋₄alkyl may optionally be substituted one or more times with fluoro;
R²⁰ is independently selected from hydrogen and fluoro;
n1 is selected from 0 and 1;
n2 is selected from 1 and 2;
X⁶ is CH or N;
X⁷ is selected from N and CH;
and
LBM is selected from 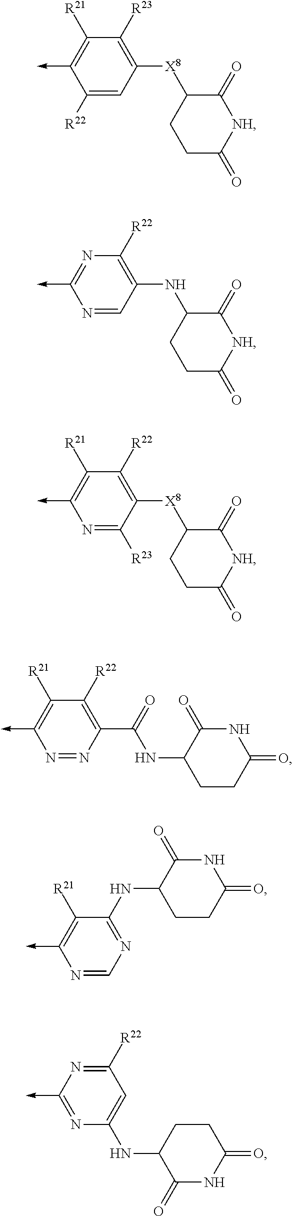 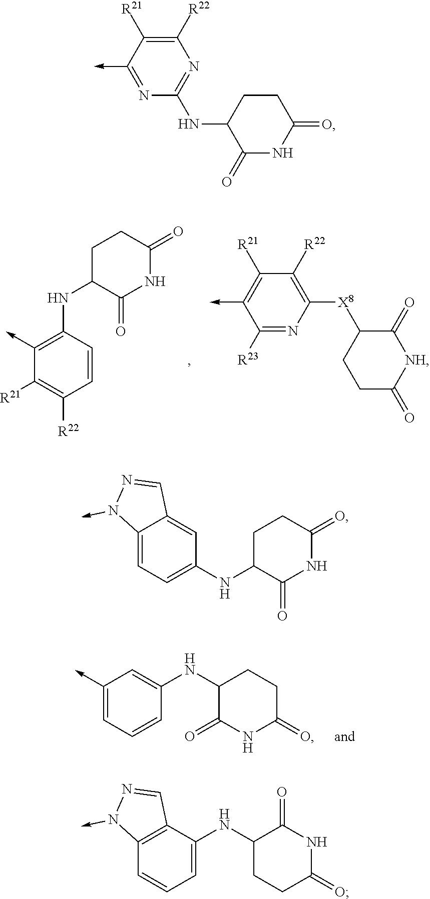
wherein
X⁸ is selected from O, NH, and triazolyl;
R²¹ is selected from hydrogen, fluoro, chloro, cyano, C₁₋₄alkyl and trifluoromethyl;
R²² is selected from hydrogen and fluoro;
R²³ is selected from hydrogen, fluoro and C₁₋₄alkoxy;
or a pharmaceutically acceptable salt or stereoisomer thereof.

2. The compound of claim 1, wherein the compound is according to formula (I′): 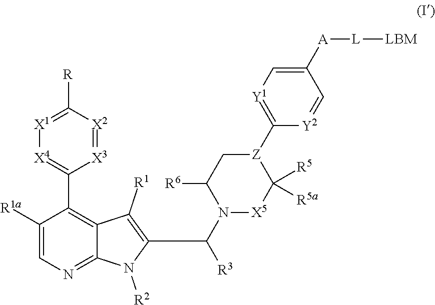
wherein:
X¹ is selected from CR¹¹ and N; X² is selected from CR¹² and N; X³ is selected from CR¹³ and N, and X⁴ is selected from CR¹⁴ and N, and wherein when X⁴ is N the N may be oxidized to a N-oxide;
X⁵ is CO or CR⁴R⁴;
Y¹ is selected from N and CR⁷;
Y² is selected from N and CR⁸;
Z is selected from N and CR¹⁰;
R is NHR⁰;
R⁰ is selected from hydrogen, C₁₋₄alkyl, C₃₋₄cycloalkyl, C₃₋₄cycloalkyl-C₁₋₄alkyl, phenyl-C₁₋₄alkyl, halo-C₁₋₄alkyl, —(CH₂)ₙ—O—R′, —(CH₂)ₙ—CO—C₁₋₄alkyl and —(CH₂)ₙ—CO₂R′,
wherein said phenyl is optionally substituted one or more times with substituents independently selected from halogen, C₁₋₄alkyl, halo-C₁₋₄alkyl, C₃₋₄ cycloalkyl, hydroxy, C₁₋₄alkoxy, cyano, —NR′R″, —CONR′R″, and —CO₂R′, wherein R′ and R″ are each independently selected from hydrogen and C₁₋₄alkyl and n is 0, 1, or 2;
R¹ and R^{1a} are independently selected from hydrogen, fluoro, and C₁₋₄alkyl;
R² is selected from hydrogen, C₁₋₅alkyl, and deuterated C₁₋₄alkyl, wherein said C₁₋₅alkyl may optionally be substituted one or more times with fluoro;
R³ is selected from hydrogen, C₁₋₄alkyl and deuterated C₁₋₄alkyl;
each of R⁴ and R⁶ are independently selected from hydrogen, C₁₋₄alkyl, and C₁₋₄alkoxy, wherein said C₁₋₄alkyl and C₁₋₄alkoxy may optionally be substituted with one or more halogen;
R⁵ is selected from hydrogen, fluoro, and C₁₋₄alkyl; wherein said C₁₋₄alkyl may optionally be substituted with one or more halogen, and R^{5a} and R¹⁰ is hydrogen; or
R⁵ and R^{5a} are both selected from fluoro and R¹⁰ is hydrogen; or
R⁵ and R¹⁰ together form a bond between the two carbon atoms to which they are attached, and R^{5a} is hydrogen;
R⁷ and R⁸ are independently selected from hydrogen, halogen, C₁₋₄alkyl, and C₁₋₄alkoxy, wherein said C₁₋₄alkyl and C₁₋₄alkoxy may optionally be substituted with one or more halogen;
R¹¹, R¹², R¹³ and R¹⁴ are independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′, —CO—(CH₂)ₘOH, tetrazolyl and —SO₂—C₁₋₄alkyl, wherein said C₁₋₄alkyl and C₁₋₄alkoxy is optionally substituted one or more times with a substituent independently selected from halogen, hydroxy, and C₁₋₄alkoxy, wherein m is 1, 2 or 3;
R¹¹ and R⁰ may together form a 5-6 membered heteroaromatic ring or 5-6 membered heterocyclic ring containing one or two N atoms, wherein said 5-6 membered heteroaromatic ring or 5-6 membered heterocyclic containing one or two N atoms is optionally be substituted one or more times with a substituent independently selected from halogen, C₁₋₄alkyl, halo-C₁₋₄alkyl, C34 cycloalkyl, hydroxy, C₁₋₄alkoxy, cyano, —COF₃, —NR′R″, —CONR′R″, and —CO₂R′, wherein said C₁₋₄alkyl may optionally be substituted one or more times with a substituent independently selected from halogen and C₁₋₄alkoxy;
A is CO or 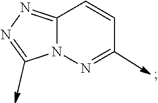
L is selected from: 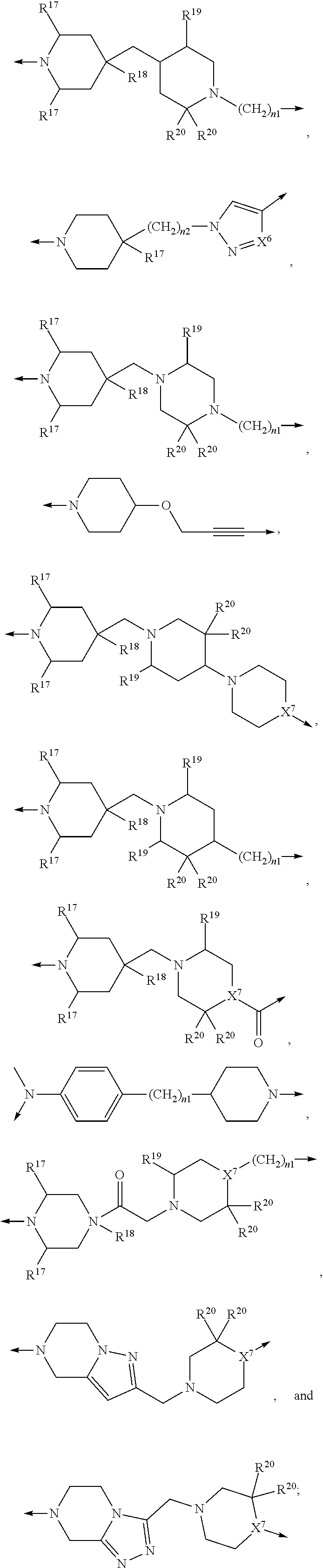
wherein
R¹⁷ is selected from hydrogen and fluoro;
R¹⁸ is selected from hydrogen and fluoro;
R¹⁹ is selected from hydrogen and C₁₋₄alkyl, wherein said C₁₋₄alkyl may optionally be substituted one or more times with fluoro;
R²⁰ is independently selected from hydrogen and fluoro;
n1 is selected from 0 and 1;
n2 is selected from 1 and 2;
X⁶ is CH or N;
X⁷ is selected from N and CH;
and
LBM is selected from 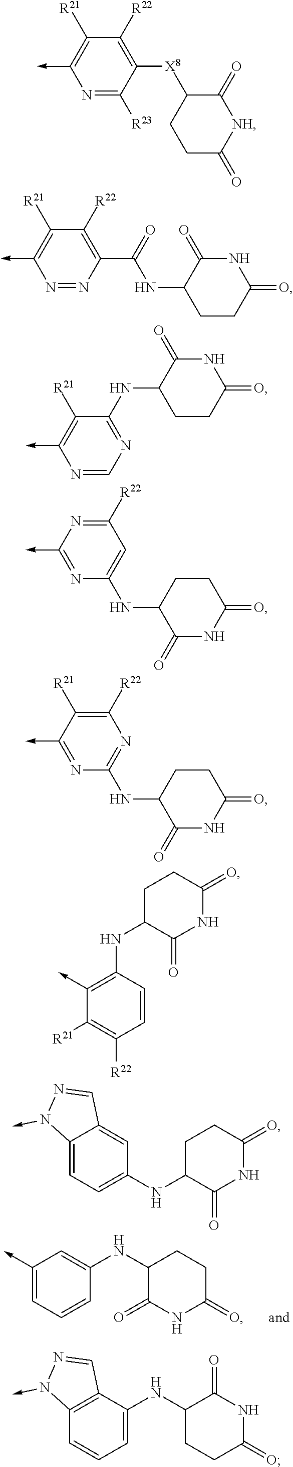
wherein
X⁸ is selected from O, NH, and triazolyl;
R²¹ is selected from hydrogen, fluoro, chloro, cyano, C₁₋₄alkyl and trifluoromethyl;
R²² is selected from hydrogen and fluoro;
R²³ is selected from hydrogen, fluoro and C₁₋₄alkoxy;
or a pharmaceutically acceptable salt or stereoisomer thereof.

3. The compound according to claim 2, having the formula (II): 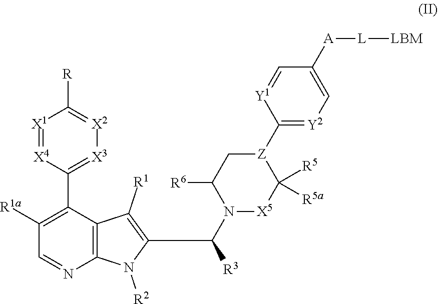
wherein R³ is C₁₋₄alkyl, or a pharmaceutically acceptable salt or stereoisomer thereof.

4. The compound according to claim 1, wherein the compound is of formula (I-b), (II-b), or (III-b): 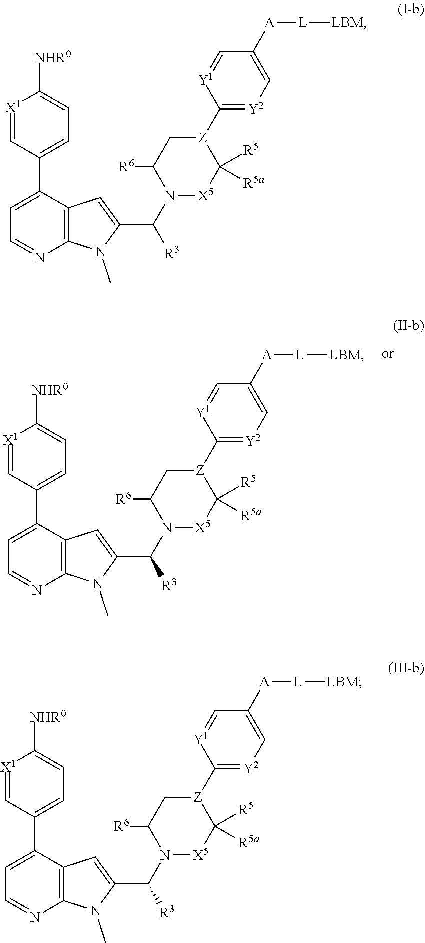
wherein R³ is C₁₋₄alkyl;
or a pharmaceutically acceptable salt or stereoisomer thereof.

5. The compound according to claim 1, wherein L is

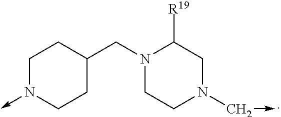

6. The compound according to claim 1, wherein L

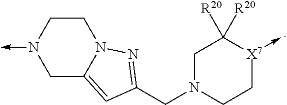

7. The compound according to claim 1, wherein LBM is

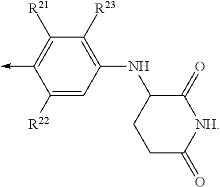

8. The compound according to claim 1, wherein A-L-LBM is:

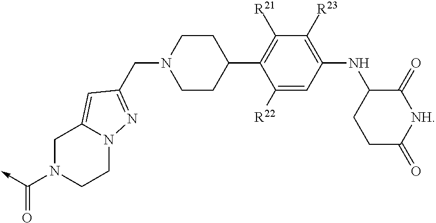

9. The compound according to claim 1, wherein A-L-LBM is

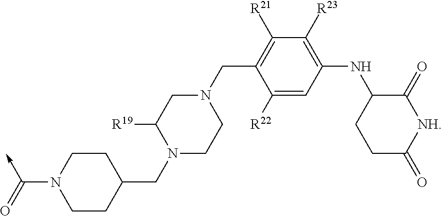

10. The compound according to claim 8, wherein R²¹ is fluoro and R²² and R²³ are hydrogen.

11. The compound according to claim 1, wherein X¹ is N, X² is CR¹², X³ is CR¹³ and X⁴ is CR 14.

12. The compound according to claim 1, wherein R is NHR⁰ and R⁰ is selected from hydrogen and C₁₋₄alkyl.

13. The compound according to claim 1, wherein R¹¹ and R⁰ together with the atoms attached thereto form a 5-6 membered heteroaromatic ring containing one or two N atoms, wherein said 5-6 membered heteroaromatic ring containing one or two N atoms is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —COF₃, —NR′R″, —CONR′R″ and —CO₂R′, wherein said C₁₋₄alkyl may optionally be substituted one or more times with a substituent independently selected from halogen and C₁₋₄alkoxy.

14. The compound according to claim 1, wherein X¹ is CH, X² is CH, X³ is CH and X⁴ is CH and R⁰ and R¹¹ together form a pyrazole ring or imidazole ring; where said pyrazole or imidazole ring may optionally be substituted by a substituent independently selected from halogen, C₁₋₄alkyl, halo-C₁₋₄alkyl, C₃₋₄ cycloalkyl, hydroxy, C₁₋₄alkoxy, cyano, —NR′R″, —CONR′R″ and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl.

15. The compound according to claim 14, wherein said pyrazole or imidazole is either unsubstituted or substituted by C₁₋₄alkoxy-C₁₋₄alkyl.

16. The compound according to claim 1, wherein Z is CR¹⁰ and R¹⁰ is hydrogen.

17. The compound according to claim 1, wherein Y¹ is CR⁷ and Y² is CR⁸.

18. The compound according to claim 17, wherein one or both of R⁷ and R⁸ is hydrogen.

19. The compound according to claim 1, wherein R¹ is hydrogen and R² and R³ is methyl.

20. The compound according to claim 1, which is selected from:

| Example | Structure |
|---|---|
| 2a1 | 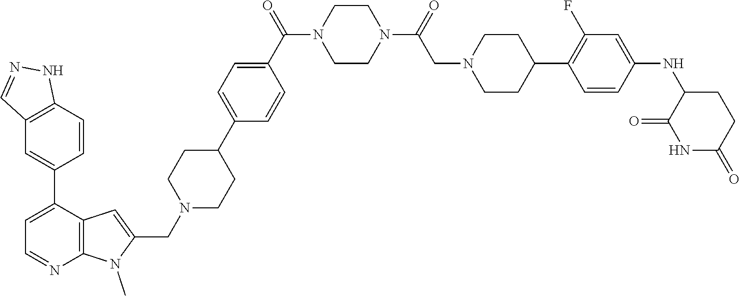 [SMILES: Cn1c(CN2CCC(c3ccc(C(=O)N4CCN(C(=O)CN5CCC(c6ccc(NC7CCC(=O)NC7=O)cc6F)CC5)CC4)cc3)CC2)cc2c(-c3ccc4[nH]ncc4c3)ccnc21] |
| 2a2 | 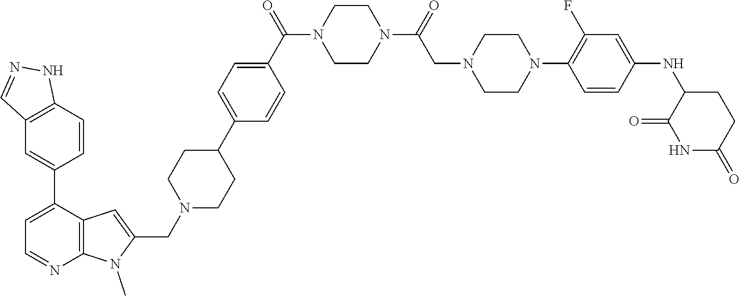 [SMILES: Cn1c(CN2CCC(c3ccc(C(=O)N4CCN(C(=O)CN5CCN(c6ccc(NC7CCC(=O)NC7=O)cc6F)CC5)CC4)cc3)CC2)cc2c(-c3ccc4[nH]ncc4c3)ccnc21] |
| 2b1 | 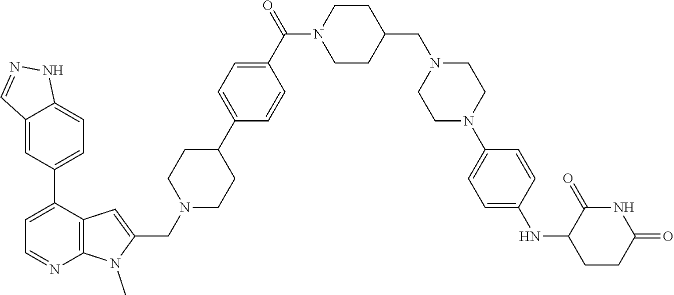 [SMILES: Cn1c(CN2CCC(c3ccc(C(=O)N4CCC(CN5CCN(c6ccc(NC7CCC(=O)NC7=O)cc6)CC5)CC4)cc3)CC2)cc2c(-c3ccc4[nH]ncc4c3)ccnc21] |
| 2b2 | 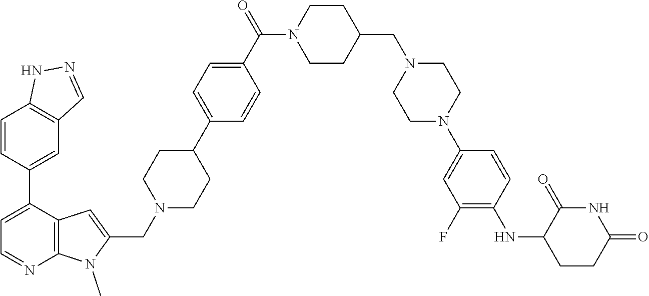 [SMILES: Cn1c(CN2CCC(c3ccc(C(=O)N4CCC(CN5CCN(c6ccc(NC7CCC(=O)NC7=O)c(F)c6)CC5)CC4)cc3)CC2)cc2c(-c3ccc4[nH]ncc4c3)ccnc21] |
| 2b3 | 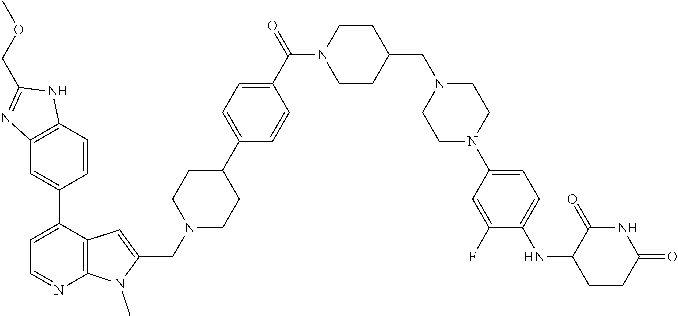 [SMILES: COCc1nc2cc(-c3ccnc4c3cc(CN3CCC(c5ccc(C(=O)N6CCC(CN7CCN(c8ccc(NC9CCC(=O)NC9=O)c(F)c8)CC7)CC6)cc5)CC3)n4C)ccc2[nH]1] |
| 2c1 | 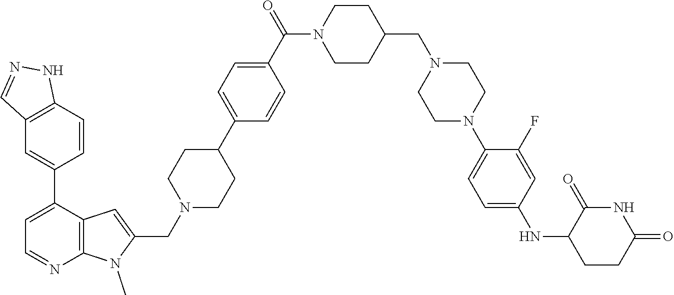 [SMILES: Cn1c(CN2CCC(c3ccc(C(=O)N4CCC(CN5CCN(c6ccc(NC7CCC(=O)NC7=O)cc6F)CC5)CC4)cc3)CC2)cc2c(-c3ccc4[nH]ncc4c3)ccnc21] |
| 2c2 | 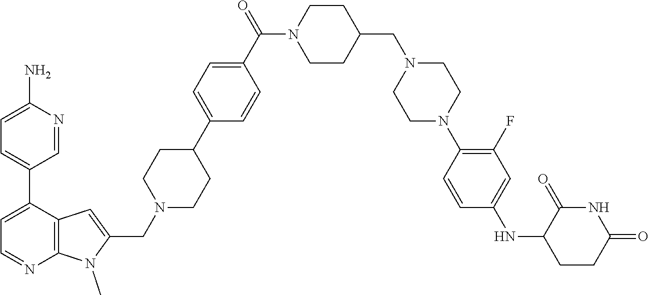 [SMILES: Cn1c(CN2CCC(c3ccc(C(=O)N4CCC(CN5CCN(c6ccc(NC7CCC(=O)NC7=O)cc6F)CC5)CC4)cc3)CC2)cc2c(-c3ccc(N)nc3)ccnc21] |
| 2c3 | 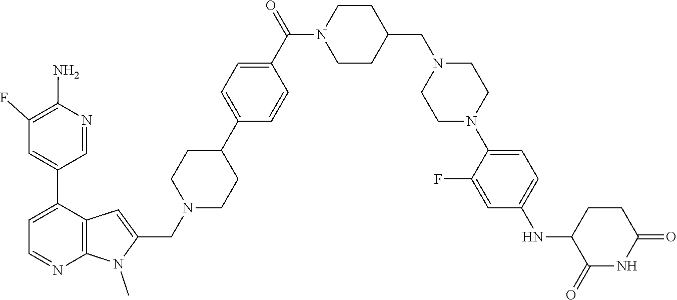 [SMILES: Cn1c(CN2CCC(c3ccc(C(=O)N4CCC(CN5CCN(c6ccc(NC7CCC(=O)NC7=O)cc6F)CC5)CC4)cc3)CC2)cc2c(-c3cnc(N)c(F)c3)ccnc21] |
| 2c4 | 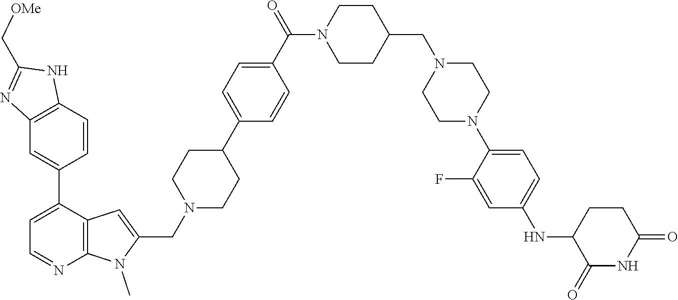 [SMILES: COCc1nc2cc(-c3ccnc4c3cc(CN3CCC(c5ccc(C(=O)N6CCC(CN7CCN(c8ccc(NC9CCC(=O)NC9=O)cc8F)CC7)CC6)cc5)CC3)n4C)ccc2[nH]1] |
| 2c5 | 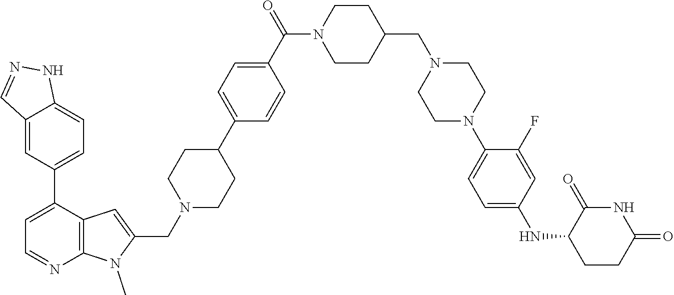 [SMILES: Cn1c(CN2CCC(c3ccc(C(=O)N4CCC(CN5CCN(c6ccc(N[C@H]7CCC(=O)NC7=O)cc6F)CC5)CC4)cc3)CC2)cc2c(-c3ccc4[nH]ncc4c3)ccnc21] |
| 2c6 | 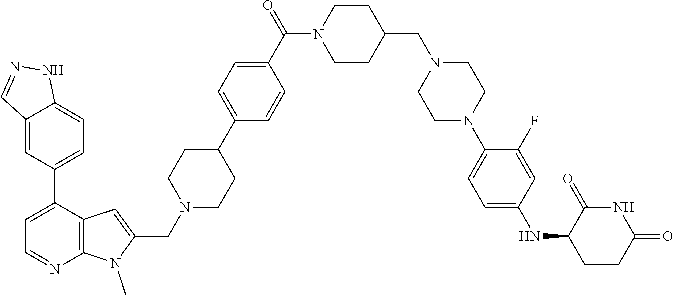 [SMILES: Cn1c(CN2CCC(c3ccc(C(=O)N4CCC(CN5CCN(c6ccc(N[C@@H]7CCC(=O)NC7=O)cc6F)CC5)CC4)cc3)CC2)cc2c(-c3ccc4[nH]ncc4c3)ccnc21] |
| 2c7 | 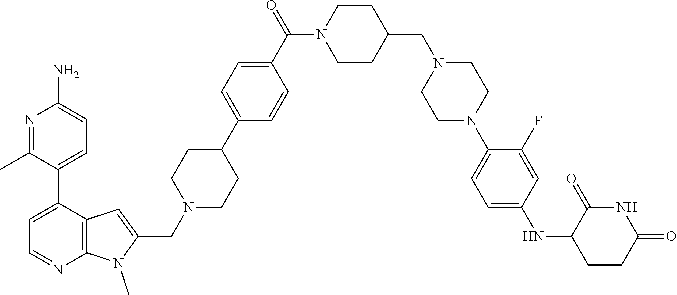 [SMILES: Cc1nc(N)ccc1-c1ccnc2c1cc(CN1CCC(c3ccc(C(=O)N4CCC(CN5CCN(c6ccc(NC7CCC(=O)NC7=O)cc6F)CC5)CC4)cc3)CC1)n2C] |
| 2c8 | 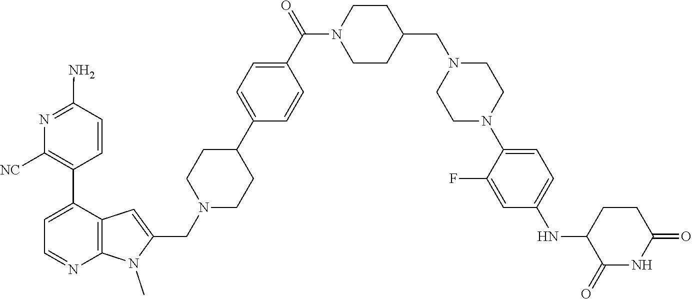 [SMILES: Cn1c(CN2CCC(c3ccc(C(=O)N4CCC(CN5CCN(c6ccc(NC7CCC(=O)NC7=O)cc6F)CC5)CC4)cc3)CC2)cc2c(-c3ccc(N)nc3C#N)ccnc21] |
| 2c9 | 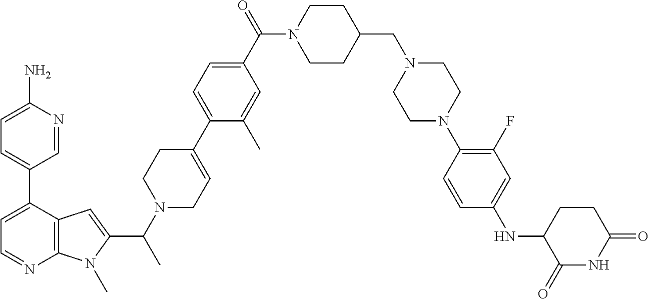 [SMILES: Cc1cc(C(=O)N2CCC(CN3CCN(c4ccc(NC5CCC(=O)NC5=O)cc4F)CC3)CC2)ccc1C1=CCN(C(C)c2cc3c(-c4ccc(N)nc4)ccnc3n2C)CC1] |
| 2c10 | 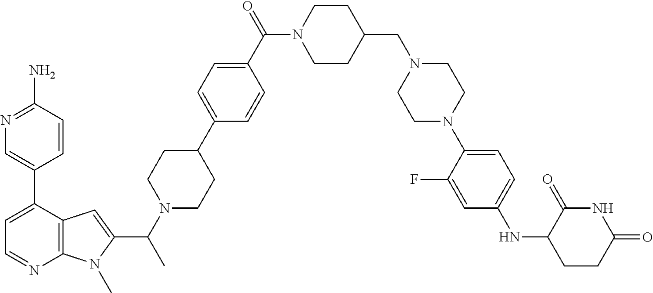 [SMILES: CC(c1cc2c(-c3ccc(N)nc3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c11 | 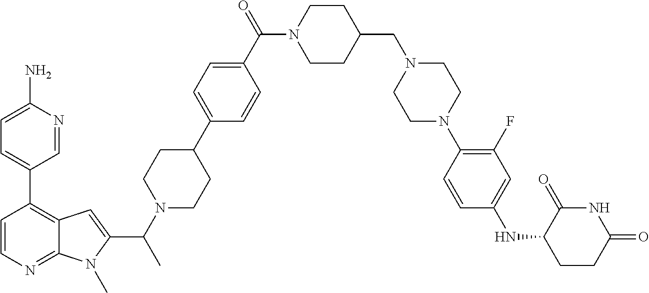 [SMILES: CC(c1cc2c(-c3ccc(N)nc3)ccnc2n1C)N1CCC(c2ccc(C(=O)N3CCC(CN4CCN(c5ccc(N[C@H]6CCC(=O)NC6=O)cc5F)CC4)CC3)cc2)CC1] |
| 2c12 | 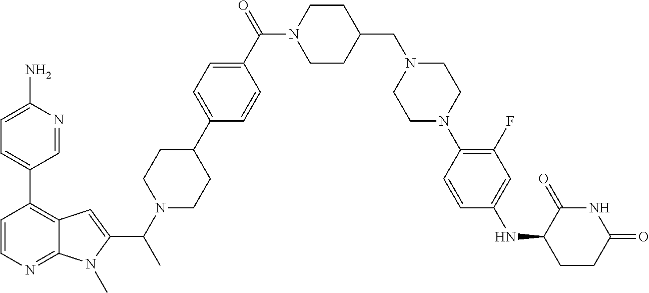 [SMILES: CC(c1cc2c(-c3ccc(N)nc3)ccnc2n1C)N1CCC(c2ccc(C(=O)N3CCC(CN4CCN(c5ccc(N[C@@H]6CCC(=O)NC6=O)cc5F)CC4)CC3)cc2)CC1] |
| 2c13 | 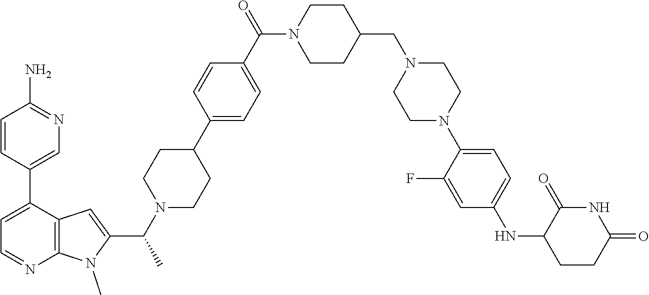 [SMILES: C[C@H](c1cc2c(-c3ccc(N)nc3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c14 | 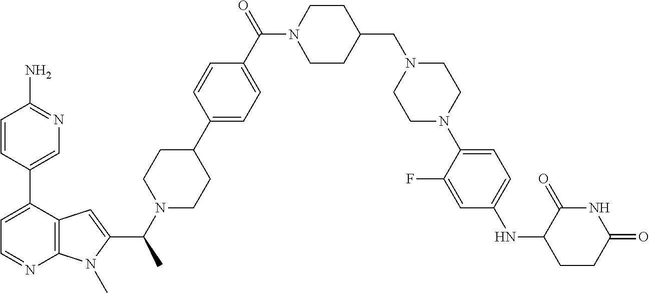 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c15 | 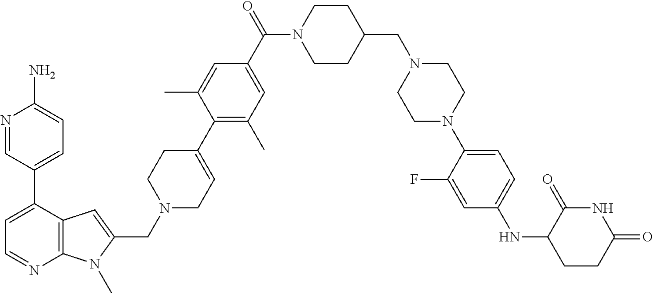 [SMILES: Cc1cc(C(=O)N2CCC(CN3CCN(c4ccc(NC5CCC(=O)NC5=O)cc4F)CC3)CC2)cc(C)c1C1=CCN(Cc2cc3c(-c4ccc(N)nc4)ccnc3n2C)CC1] |
| 2c16 | 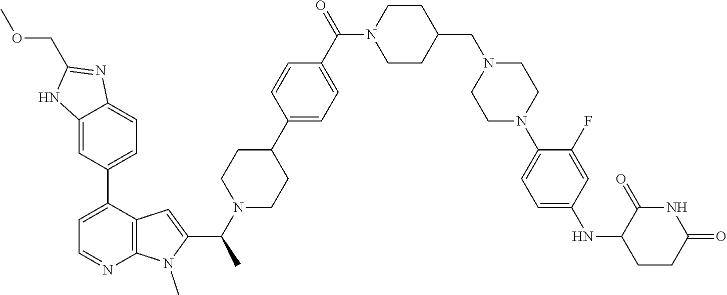 [SMILES: COCc1nc2ccc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)cc2[nH]1] |
| 2c17 | 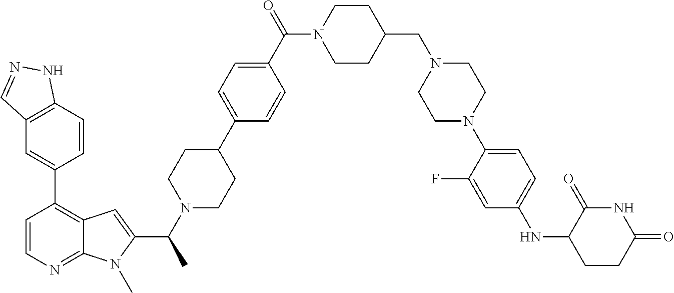 [SMILES: C[C@@H](c1cc2c(-c3ccc4[nH]ncc4c3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c18 | 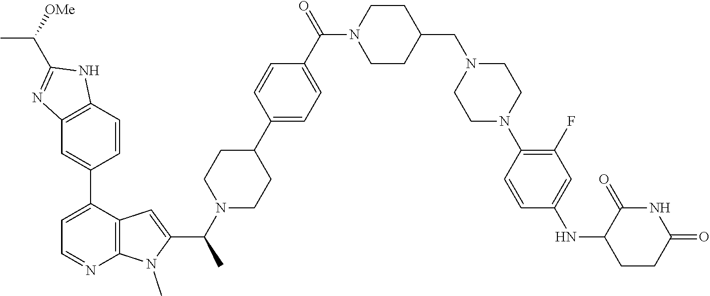 [SMILES: CO[C@@H](C)c1nc2cc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)ccc2[nH]1] |
| 2c19 | 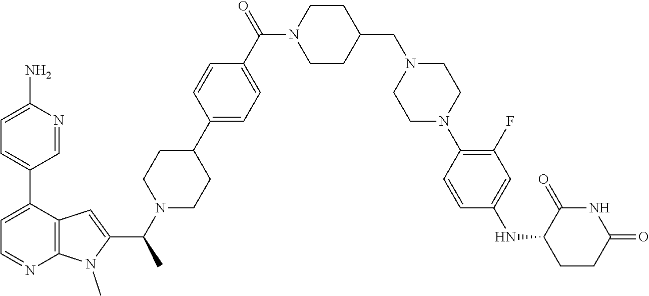 [SMILES: C[C@@H](c1cc2c(-c3ccc(N)nc3)ccnc2n1C)N1CCC(c2ccc(C(=O)N3CCC(CN4CCN(c5ccc(N[C@H]6CCC(=O)NC6=O)cc5F)CC4)CC3)cc2)CC1] |
| 2c20 | 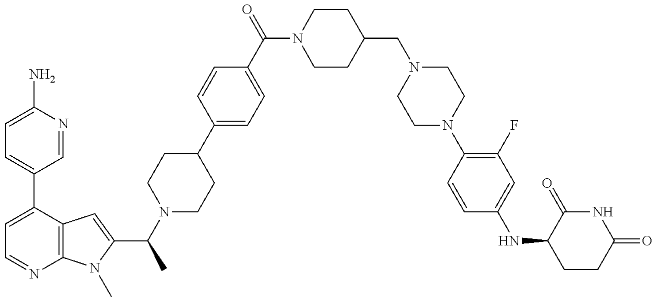 [SMILES: C[C@@H](c1cc2c(-c3ccc(N)nc3)ccnc2n1C)N1CCC(c2ccc(C(=O)N3CCC(CN4CCN(c5ccc(N[C@@H]6CCC(=O)NC6=O)cc5F)CC4)CC3)cc2)CC1] |
| 2c21 | 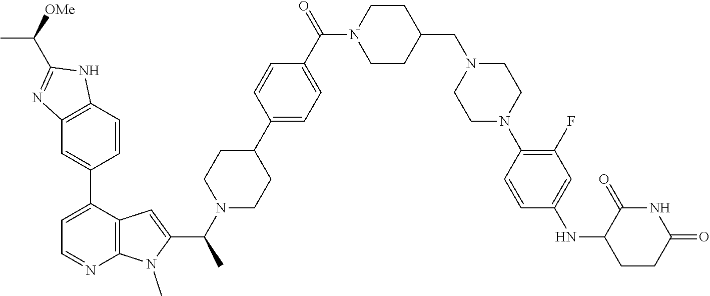 [SMILES: CO[C@H](C)c1nc2cc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)ccc2[nH]1] |
| 2c22 | 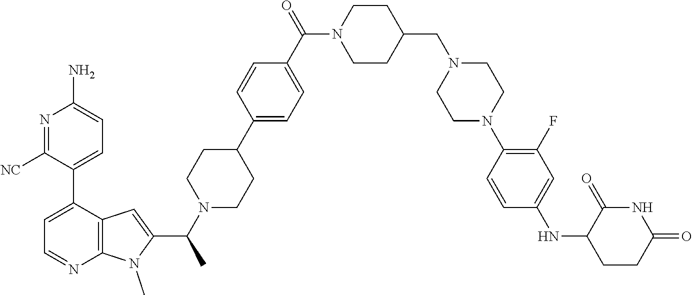 [SMILES: C[C@@H](c1cc2c(-c3ccc(N)nc3C#N)ccnc2n1C)N1CCC(c2ccc(C(=O)N3CCC(CN4CCN(c5ccc(NC6CCC(=O)NC6=O)cc5F)CC4)CC3)cc2)CC1] |
| 2c23 | 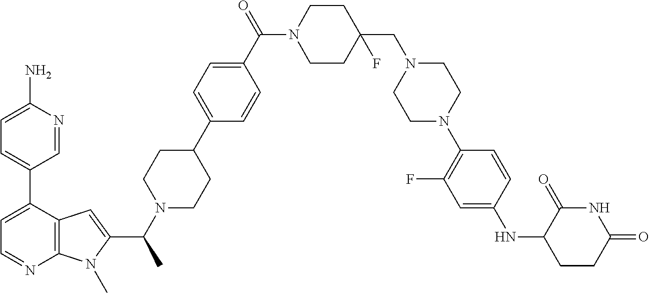 [SMILES: C[C@@H](c1cc2c(-c3ccc(N)nc3)ccnc2n1C)N1CCC(c2ccc(C(=O)N3CCC(F)(CN4CCN(c5ccc(NC6CCC(=O)NC6=O)cc5F)CC4)CC3)cc2)CC1] |
| 2c24 | 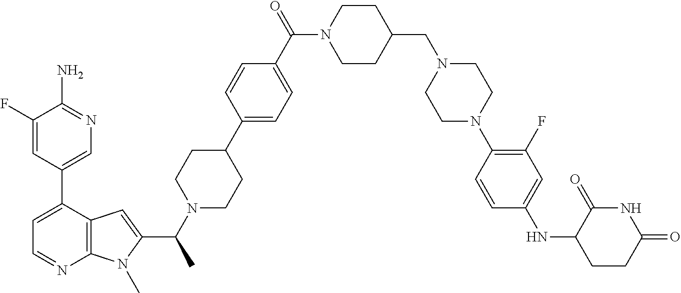 [SMILES: C[C@@H](c1cc2c(-c3cnc(N)c(F)c3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c26 | 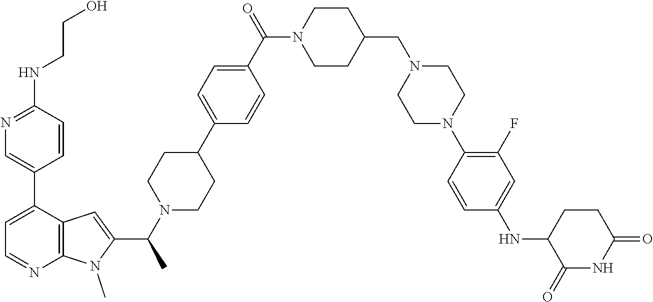 [SMILES: C[C@@H](c1cc2c(-c3ccc(NCCO)nc3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c27 | 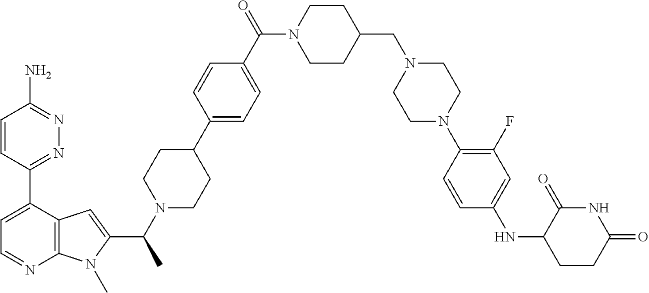 [SMILES: C[C@@H](c1cc2c(-c3ccc(N)nn3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c28 | 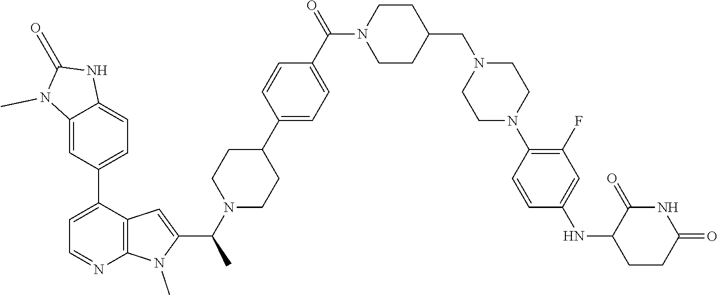 [SMILES: C[C@@H](c1cc2c(-c3ccc4[nH]c(=O)n(C)c4c3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c30 | 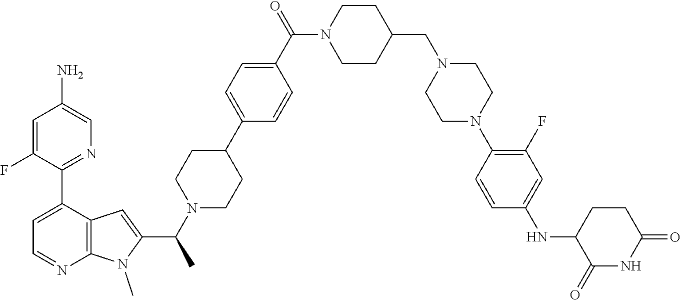 [SMILES: C[C@@H](c1cc2c(-c3ncc(N)cc3F)ccnc2n1C)N1CCC(c2ccc(C(=O)N3CCC(CN4CCN(c5ccc(NC6CCC(=O)NC6=O)cc5F)CC4)CC3)cc2)CC1] |
| 2c32 | 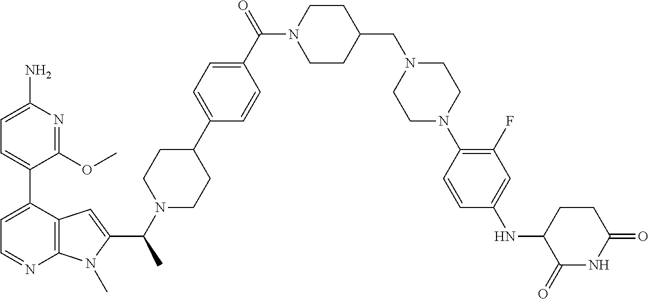 [SMILES: COc1nc(N)ccc1-c1ccnc2c1cc([C@H](C)N1CCC(c3ccc(C(=O)N4CCC(CN5CCN(c6ccc(NC7CCC(=O)NC7=O)cc6F)CC5)CC4)cc3)CC1)n2C] |
| 2c33 | 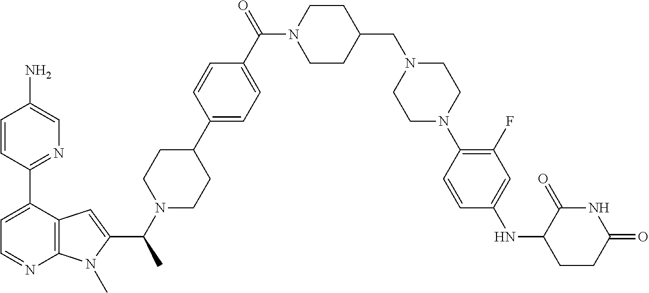 [SMILES: C[C@@H](c1cc2c(-c3ccc(N)cn3)ccnc2n1C)N1CCC(c2ccc(C(=O)N3CCC(CN4CCN(c5ccc(NC6CCC(=O)NC6=O)cc5F)CC4)CC3)cc2)CC1] |
| 2c34 | 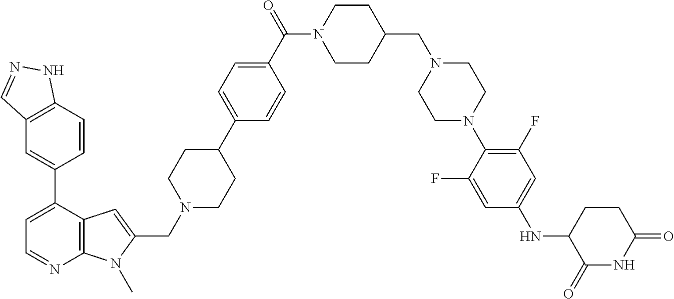 [SMILES: Cn1c(CN2CCC(c3ccc(C(=O)N4CCC(CN5CCN(c6c(F)cc(NC7CCC(=O)NC7=O)cc6F)CC5)CC4)cc3)CC2)cc2c(-c3ccc4[nH]ncc4c3)ccnc21] |
| 2c37 | 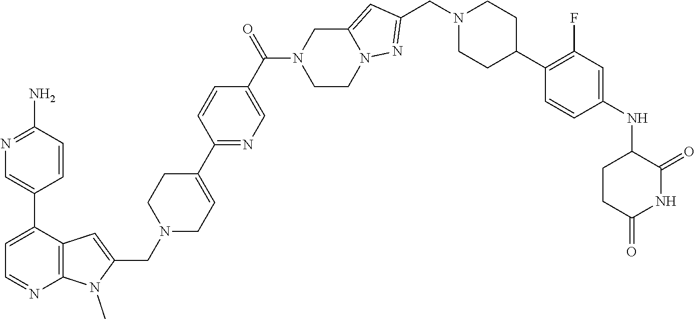 [SMILES: Cn1c(CN2CC=C(c3ccc(C(=O)N4CCn5nc(CN6CCC(c7ccc(NC8CCC(=O)NC8=O)cc7F)CC6)cc5C4)cn3)CC2)cc2c(-c3ccc(N)nc3)ccnc21] |
| 2c38 | 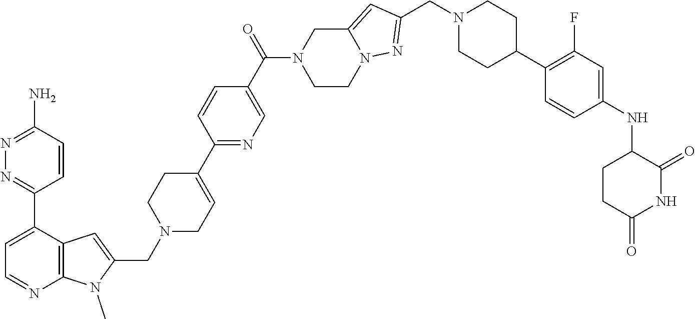 [SMILES: Cn1c(CN2CC=C(c3ccc(C(=O)N4CCn5nc(CN6CCC(c7ccc(NC8CCC(=O)NC8=O)cc7F)CC6)cc5C4)cn3)CC2)cc2c(-c3ccc(N)nn3)ccnc21] |
| 2c39 | 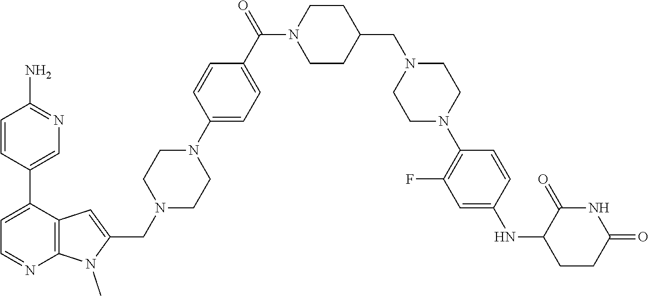 [SMILES: Cn1c(CN2CCN(c3ccc(C(=O)N4CCC(CN5CCN(c6ccc(NC7CCC(=O)NC7=O)cc6F)CC5)CC4)cc3)CC2)cc2c(-c3ccc(N)nc3)ccnc21] |
| 2c40 | 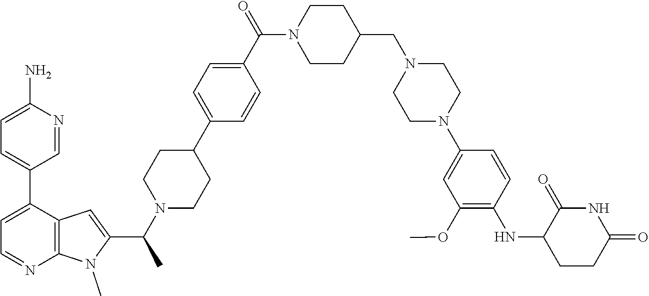 [SMILES: COc1cc(N2CCN(CC3CCN(C(=O)c4ccc(C5CCN([C@@H](C)c6cc7c(-c8ccc(N)nc8)ccnc7n6C)CC5)cc4)CC3)CC2)ccc1NC1CCC(=O)NC1=O] |
| 2c41 | 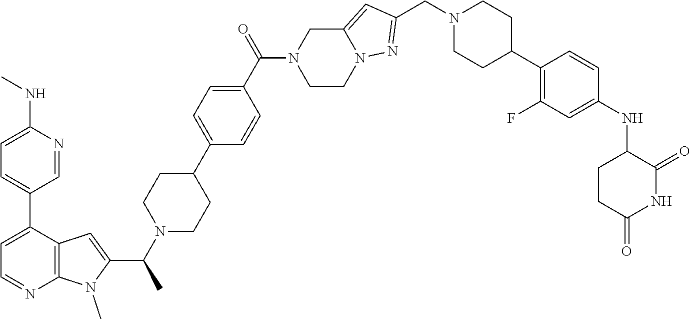 [SMILES: CNc1ccc(-c2ccnc3c2cc([C@H](C)N2CCC(c4ccc(C(=O)N5CCn6nc(CN7CCC(c8ccc(NC9CCC(=O)NC9=O)cc8F)CC7)cc6C5)cc4)CC2)n3C)cn1] |
| 2c42 | 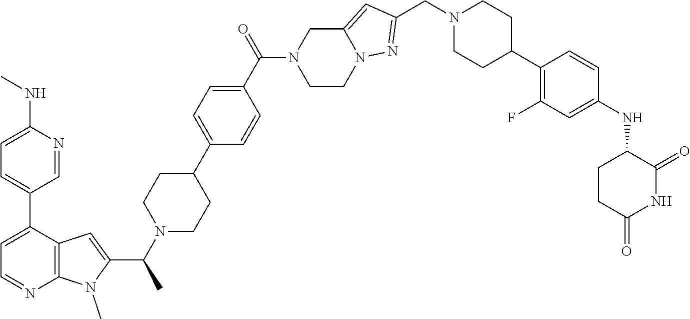 [SMILES: CNc1ccc(-c2ccnc3c2cc([C@H](C)N2CCC(c4ccc(C(=O)N5CCn6nc(CN7CCC(c8ccc(N[C@H]9CCC(=O)NC9=O)cc8F)CC7)cc6C5)cc4)CC2)n3C)cn1] |
| 2c43 | 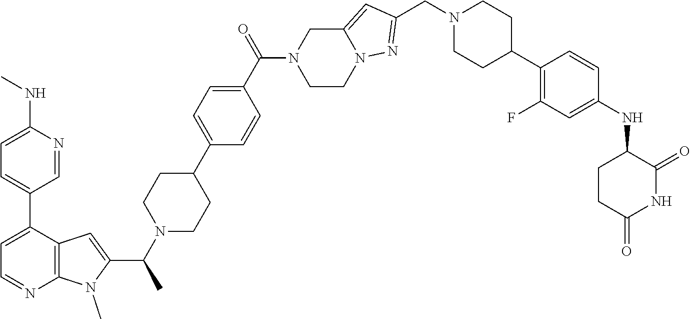 [SMILES: CNc1ccc(-c2ccnc3c2cc([C@H](C)N2CCC(c4ccc(C(=O)N5CCn6nc(CN7CCC(c8ccc(N[C@@H]9CCC(=O)NC9=O)cc8F)CC7)cc6C5)cc4)CC2)n3C)cn1] |
| 2c44 | 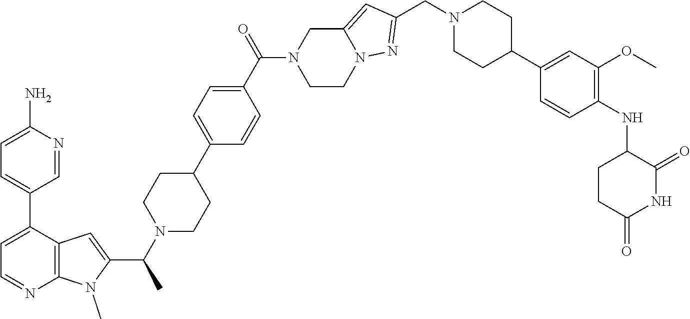 [SMILES: COc1cc(C2CCN(Cc3cc4n(n3)CCN(C(=O)c3ccc(C5CCN([C@@H](C)c6cc7c(-c8ccc(N)nc8)ccnc7n6C)CC5)cc3)C4)CC2)ccc1NC1CCC(=O)NC1=O] |
| 2c45 | 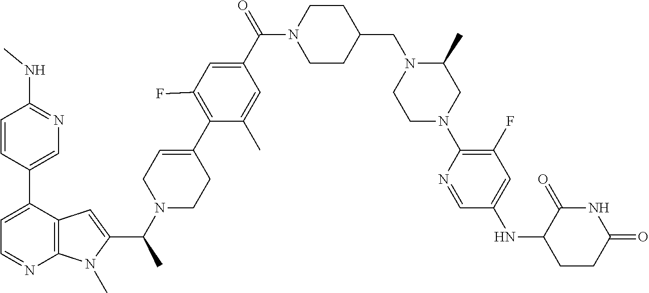 [SMILES: CNc1ccc(-c2ccnc3c2cc([C@H](C)N2CC=C(c4c(C)cc(C(=O)N5CCC(CN6CCN(c7ncc(NC8CCC(=O)NC8=O)cc7F)C[C@@H]6C)CC5)cc4F)CC2)n3C)cn1] |
| 2c46 | 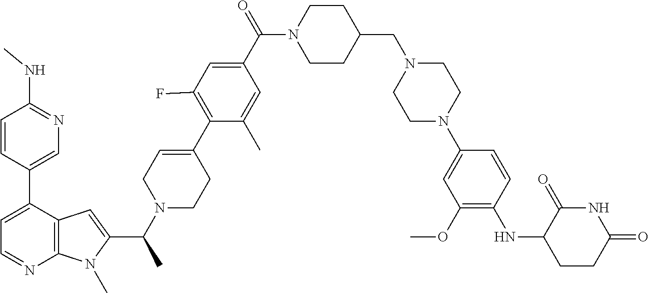 [SMILES: CNc1ccc(-c2ccnc3c2cc([C@H](C)N2CC=C(c4c(C)cc(C(=O)N5CCC(CN6CCN(c7ccc(NC8CCC(=O)NC8=O)c(OC)c7)CC6)CC5)cc4F)CC2)n3C)cn1] |
| 2c47 | 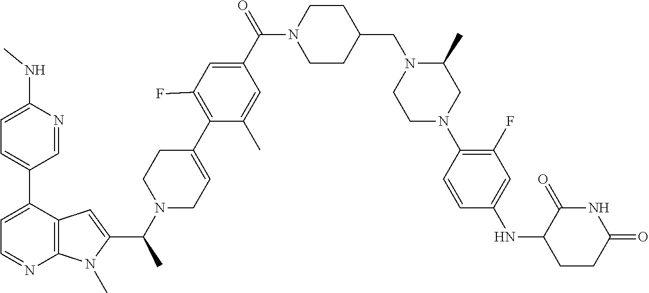 [SMILES: CNc1ccc(-c2ccnc3c2cc([C@H](C)N2CC=C(c4c(C)cc(C(=O)N5CCC(CN6CCN(c7ccc(NC8CCC(=O)NC8=O)cc7F)C[C@@H]6C)CC5)cc4F)CC2)n3C)cn1] |
| 2c48 | 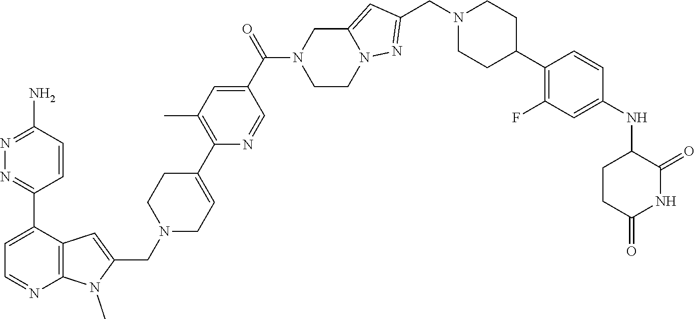 [SMILES: Cc1cc(C(=O)N2CCn3nc(CN4CCC(c5ccc(NC6CCC(=O)NC6=O)cc5F)CC4)cc3C2)cnc1C1=CCN(Cc2cc3c(-c4ccc(N)nn4)ccnc3n2C)CC1] |
| 2c49 | 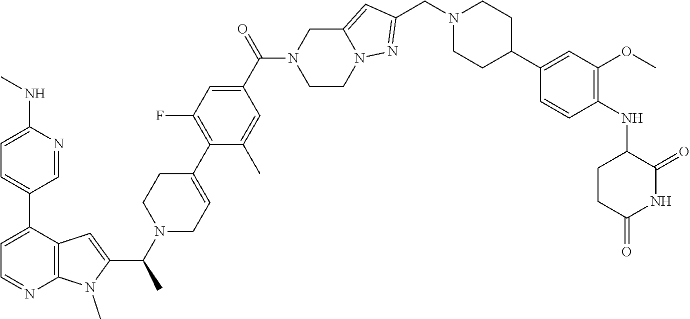 [SMILES: CNc1ccc(-c2ccnc3c2cc([C@H](C)N2CC=C(c4c(C)cc(C(=O)N5CCn6nc(CN7CCC(c8ccc(NC9CCC(=O)NC9=O)c(OC)c8)CC7)cc6C5)cc4F)CC2)n3C)cn1] |
| 2c52 | 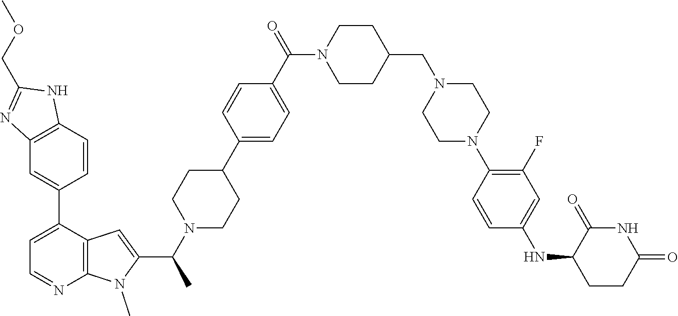 [SMILES: COCc1nc2cc(-c3ccnc4c3cc([C@H](C)N3CCC(c5ccc(C(=O)N6CCC(CN7CCN(c8ccc(N[C@@H]9CCC(=O)NC9=O)cc8F)CC7)CC6)cc5)CC3)n4C)ccc2[nH]1] |
| 2c53 | 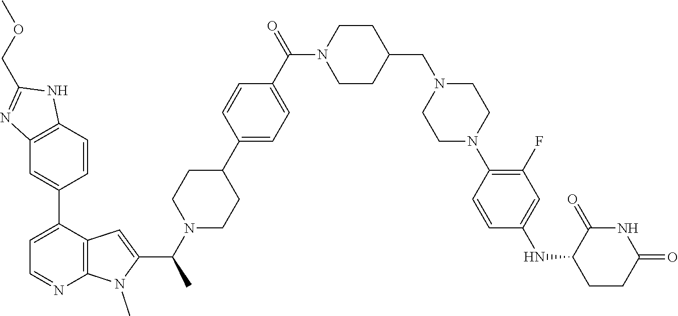 [SMILES: COCc1nc2cc(-c3ccnc4c3cc([C@H](C)N3CCC(c5ccc(C(=O)N6CCC(CN7CCN(c8ccc(N[C@H]9CCC(=O)NC9=O)cc8F)CC7)CC6)cc5)CC3)n4C)ccc2[nH]1] |
| 2c54 | 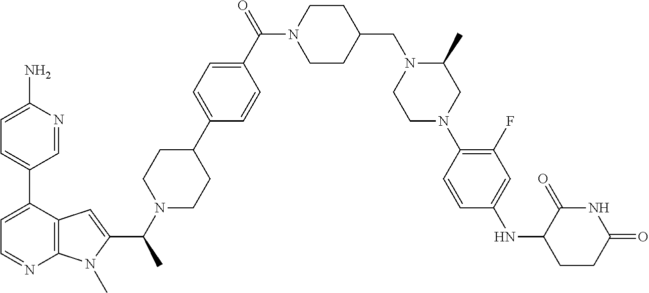 [SMILES: C[C@H]1CN(c2ccc(NC3CCC(=O)NC3=O)cc2F)CCN1CC1CCN(C(=O)c2ccc(C3CCN([C@@H](C)c4cc5c(-c6ccc(N)nc6)ccnc5n4C)CC3)cc2)CC1] |
| 2c55 | 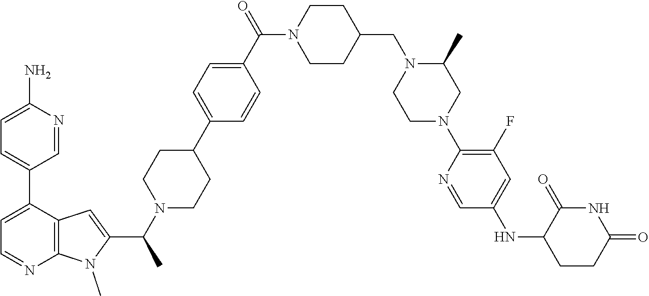 [SMILES: C[C@H]1CN(c2ncc(NC3CCC(=O)NC3=O)cc2F)CCN1CC1CCN(C(=O)c2ccc(C3CCN([C@@H](C)c4cc5c(-c6ccc(N)nc6)ccnc5n4C)CC3)cc2)CC1] |
| 2c56 | 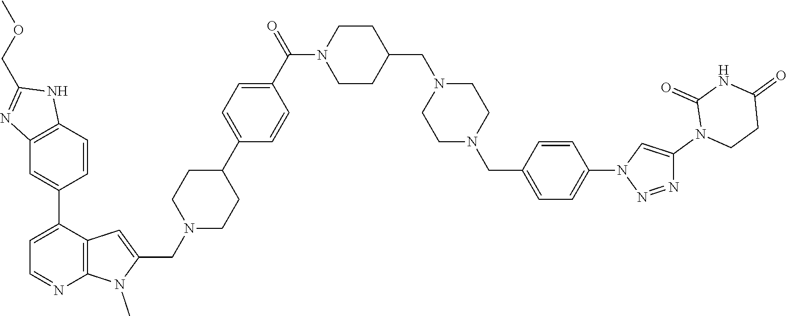 [SMILES: COCc1nc2cc(-c3ccnc4c3cc(CN3CCC(c5ccc(C(=O)N6CCC(CN7CCN(Cc8ccc(-n9cc(N%10CCC(=O)NC%10=O)nn9)cc8)CC7)CC6)cc5)CC3)n4C)ccc2[nH]1] |
| 2c57 | 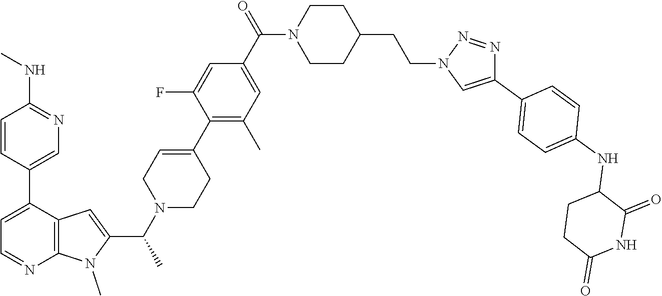 [SMILES: CNc1ccc(-c2ccnc3c2cc([C@@H](C)N2CC=C(c4c(C)cc(C(=O)N5CCC(CCn6cc(-c7ccc(NC8CCC(=O)NC8=O)cc7)nn6)CC5)cc4F)CC2)n3C)cn1] |
| 2d1 | 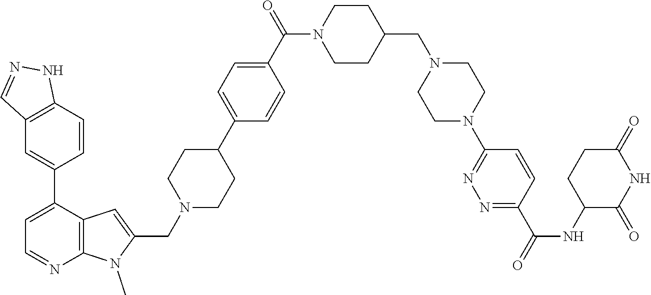 [SMILES: Cn1c(CN2CCC(c3ccc(C(=O)N4CCC(CN5CCN(c6ccc(C(=O)NC7CCC(=O)NC7=O)nn6)CC5)CC4)cc3)CC2)cc2c(-c3ccc4[nH]ncc4c3)ccnc21] |
| 2e1 | 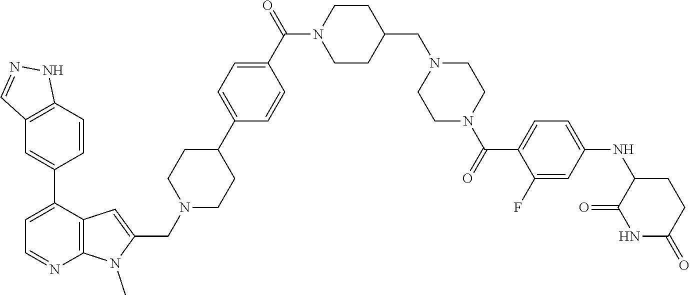 [SMILES: Cn1c(CN2CCC(c3ccc(C(=O)N4CCC(CN5CCN(C(=O)c6ccc(NC7CCC(=O)NC7=O)cc6F)CC5)CC4)cc3)CC2)cc2c(-c3ccc4[nH]ncc4c3)ccnc21] |
| 2e2 | 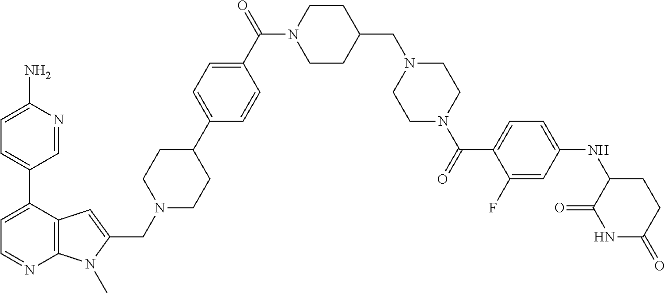 [SMILES: Cn1c(CN2CCC(c3ccc(C(=O)N4CCC(CN5CCN(C(=O)c6ccc(NC7CCC(=O)NC7=O)cc6F)CC5)CC4)cc3)CC2)cc2c(-c3ccc(N)nc3)ccnc21] |
| 2f1 | 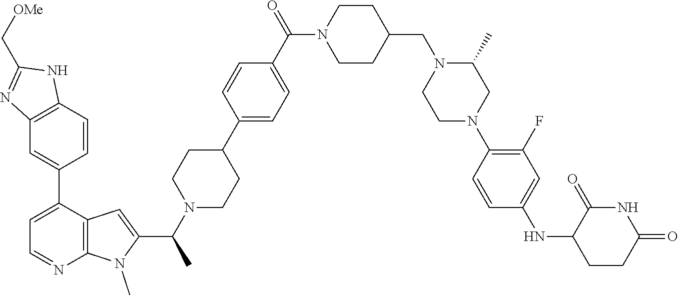 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8F)C[C@H]7C)CC6)cc5)CC3)n4C)ccc2[nH]1] |
| 2f2 | 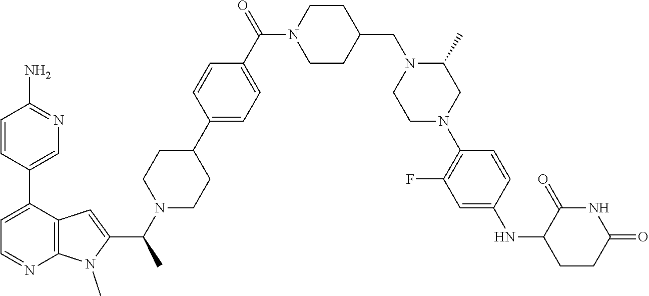 [SMILES: C[C@@H]1CN(c2ccc(NC3CCC(=O)NC3=O)cc2F)CCN1CC1CCN(C(=O)c2ccc(C3CCN([C@@H](C)c4cc5c(-c6ccc(N)nc6)ccnc5n4C)CC3)cc2)CC1] |
| 2f3 | 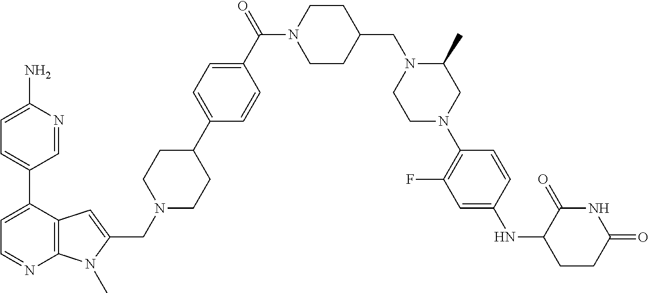 [SMILES: C[C@H]1CN(c2ccc(NC3CCC(=O)NC3=O)cc2F)CCN1CC1CCN(C(=O)c2ccc(C3CCN(Cc4cc5c(-c6ccc(N)nc6)ccnc5n4C)CC3)cc2)CC1] |
| 2f4 | 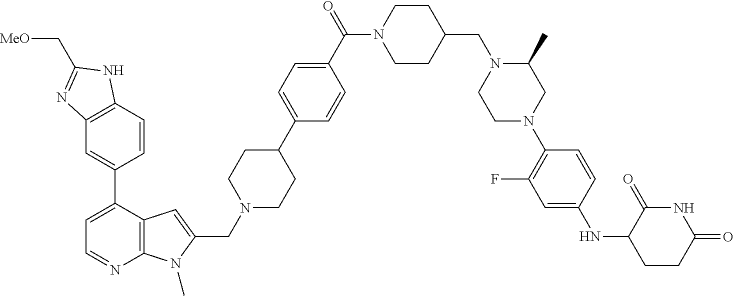 [SMILES: COCc1nc2cc(-c3ccnc4c3cc(CN3CCC(c5ccc(C(=O)N6CCC(CN7CCN(c8ccc(NC9CCC(=O)NC9=O)cc8F)C[C@@H]7C)CC6)cc5)CC3)n4C)ccc2[nH]1] |
| 2f5 | 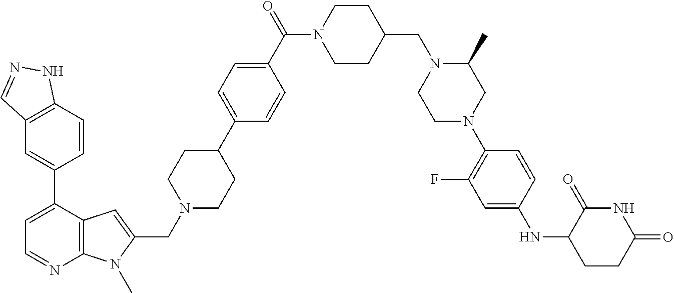 [SMILES: C[C@H]1CN(c2ccc(NC3CCC(=O)NC3=O)cc2F)CCN1CC1CCN(C(=O)c2ccc(C3CCN(Cc4cc5c(-c6ccc7[nH]ncc7c6)ccnc5n4C)CC3)cc2)CC1] |
| 2f6 | 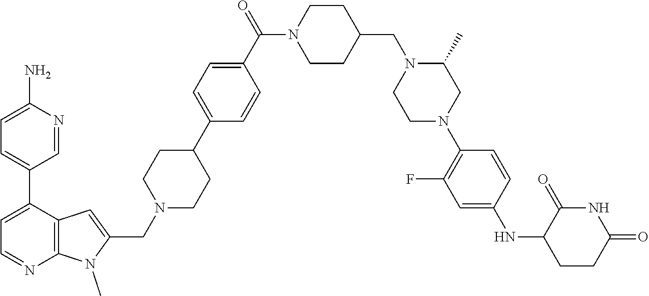 [SMILES: C[C@@H]1CN(c2ccc(NC3CCC(=O)NC3=O)cc2F)CCN1CC1CCN(C(=O)c2ccc(C3CCN(Cc4cc5c(-c6ccc(N)nc6)ccnc5n4C)CC3)cc2)CC1] |
| 2f7 | 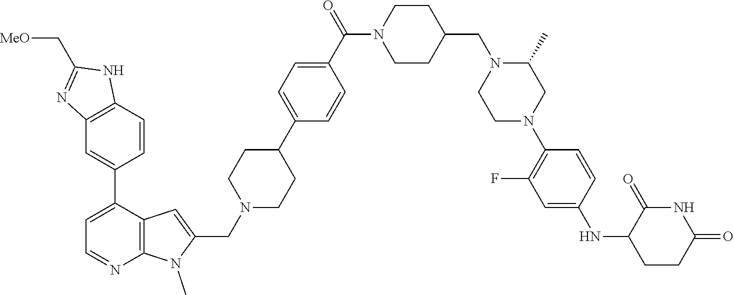 [SMILES: COCc1nc2cc(-c3ccnc4c3cc(CN3CCC(c5ccc(C(=O)N6CCC(CN7CCN(c8ccc(NC9CCC(=O)NC9=O)cc8F)C[C@H]7C)CC6)cc5)CC3)n4C)ccc2[nH]1] |
| 2f8 | 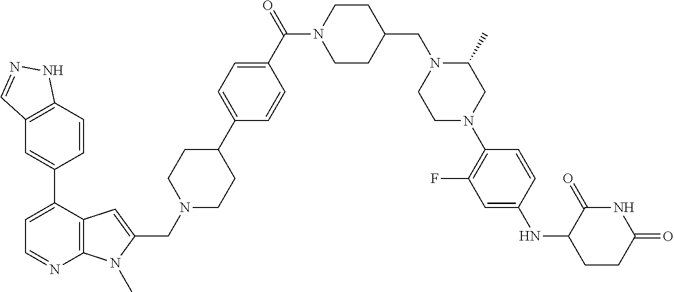 [SMILES: C[C@@H]1CN(c2ccc(NC3CCC(=O)NC3=O)cc2F)CCN1CC1CCN(C(=O)c2ccc(C3CCN(Cc4cc5c(-c6ccc7[nH]ncc7c6)ccnc5n4C)CC3)cc2)CC1] |
| 2f9 | 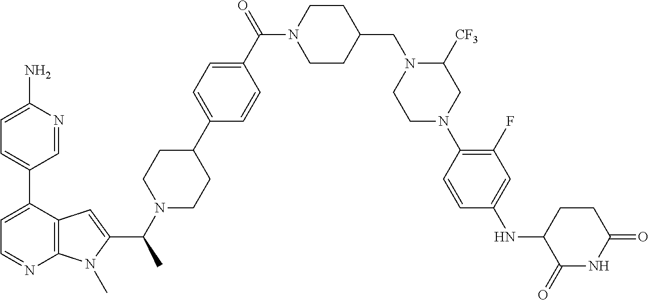 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5F)CC4C(F)(F)F)CC3)cc2)CC1] |
| 2f10 | 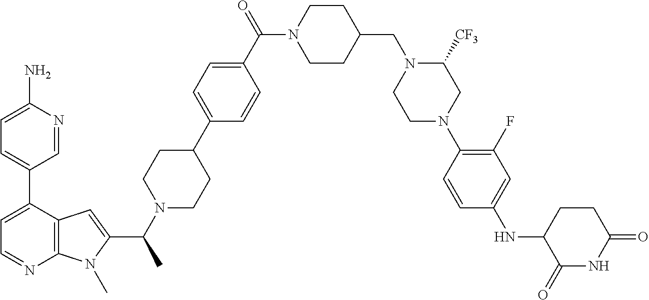 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5F)C[C@H]4C(F)(F)F)CC3)cc2)CC1] |
| 2f11 | 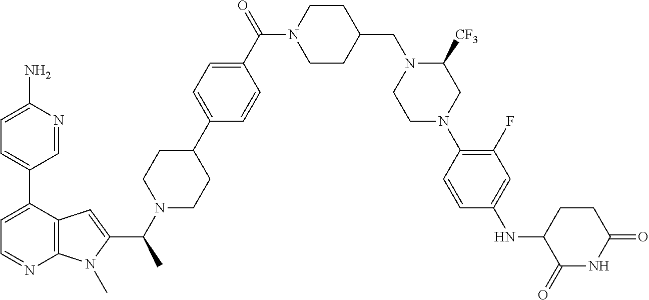 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5F)C[C@@H]4C(F)(F)F)CC3)cc2)CC1] |
| 2f12 | 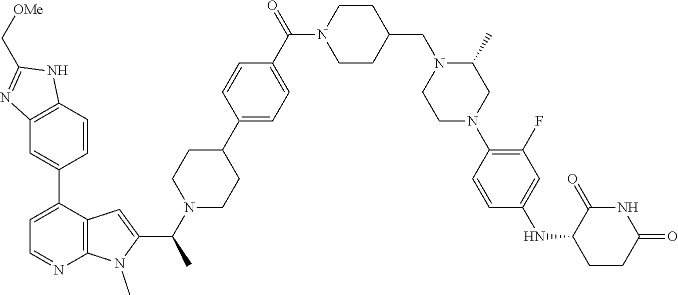 [SMILES: COCc1nc2cc(-c3ccnc4c3cc([C@H](C)N3CCC(c5ccc(C(=O)N6CCC(CN7CCN(c8ccc(N[C@H]9CCC(=O)NC9=O)cc8F)C[C@H]7C)CC6)cc5)CC3)n4C)ccc2[nH]1] |
| 2f13 | 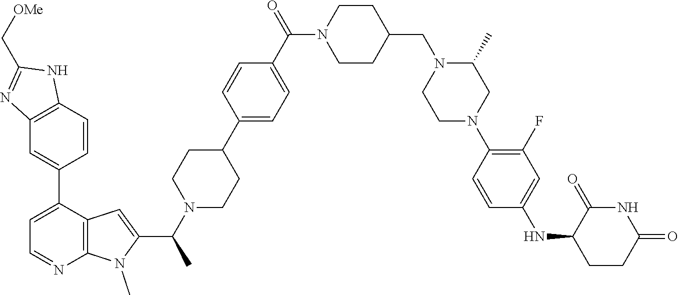 [SMILES: COCc1nc2cc(-c3ccnc4c3cc([C@H](C)N3CCC(c5ccc(C(=O)N6CCC(CN7CCN(c8ccc(N[C@@H]9CCC(=O)NC9=O)cc8F)C[C@H]7C)CC6)cc5)CC3)n4C)ccc2[nH]1] |
| 2g1 | 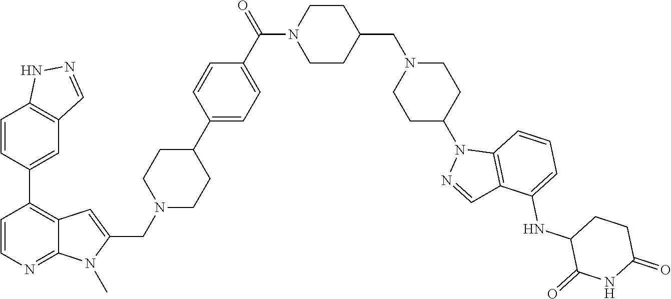 [SMILES: Cn1c(CN2CCC(c3ccc(C(=O)N4CCC(CN5CCC(n6ncc7c(NC8CCC(=O)NC8=O)cccc76)CC5)CC4)cc3)CC2)cc2c(-c3ccc4[nH]ncc4c3)ccnc21] |
| 2g63 | 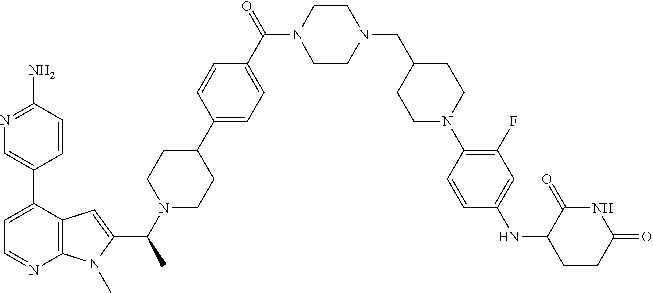 [SMILES: C[C@@H](c1cc2c(-c3ccc(N)nc3)ccnc2n1C)N1CCC(c2ccc(C(=O)N3CCN(CC4CCN(c5ccc(NC6CCC(=O)NC6=O)cc5F)CC4)CC3)cc2)CC1] |
| 2h1 | 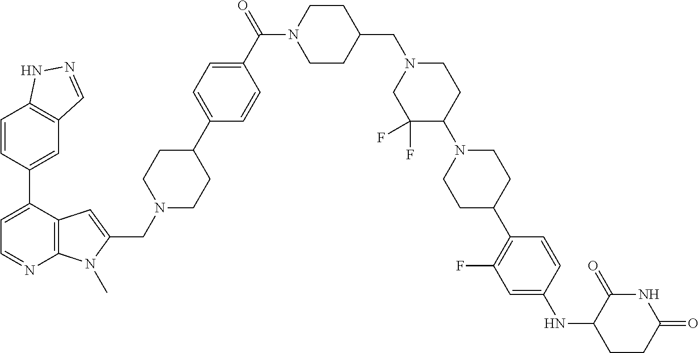 [SMILES: Cn1c(CN2CCC(c3ccc(C(=O)N4CCC(CN5CCC(N6CCC(c7ccc(NC8CCC(=O)NC8=O)cc7F)CC6)C(F)(F)C5)CC4)cc3)CC2)cc2c(-c3ccc4[nH]ncc4c3)ccnc21] |
| 2h2 | 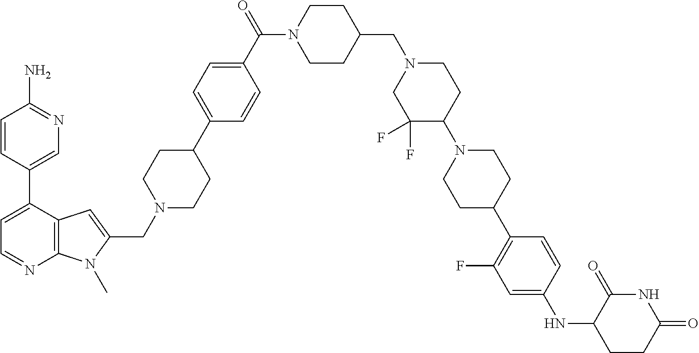 [SMILES: Cn1c(CN2CCC(c3ccc(C(=O)N4CCC(CN5CCC(N6CCC(c7ccc(NC8CCC(=O)NC8=O)cc7F)CC6)C(F)(F)C5)CC4)cc3)CC2)cc2c(-c3ccc(N)nc3)ccnc21] |
| 2h3 | 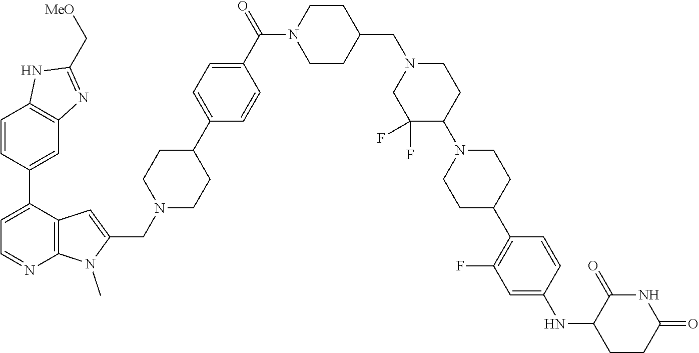 [SMILES: COCc1nc2cc(-c3ccnc4c3cc(CN3CCC(c5ccc(C(=O)N6CCC(CN7CCC(N8CCC(c9ccc(NC%10CCC(=O)NC%10=O)cc9F)CC8)C(F)(F)C7)CC6)cc5)CC3)n4C)ccc2[nH]1] |
| 2i1 | 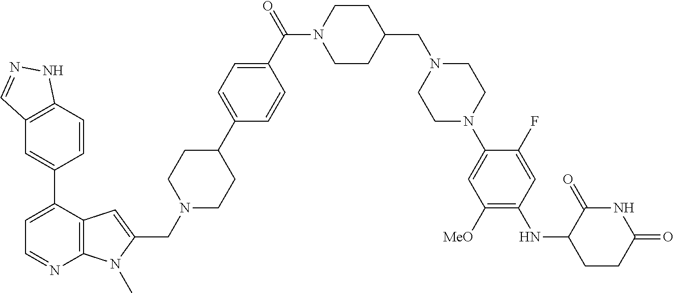 [SMILES: COc1cc(N2CCN(CC3CCN(C(=O)c4ccc(C5CCN(Cc6cc7c(-c8ccc9[nH]ncc9c8)ccnc7n6C)CC5)cc4)CC3)CC2)c(F)cc1NC1CCC(=O)NC1=O] |
| 2i2 | 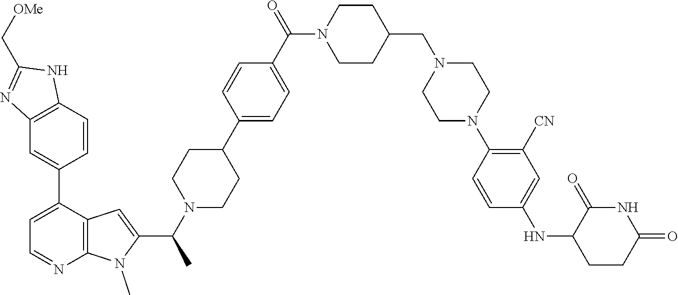 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8C#N)CC7)CC6)cc5)CC3)n4C)ccc2[nH]1] |
| 2i3 | 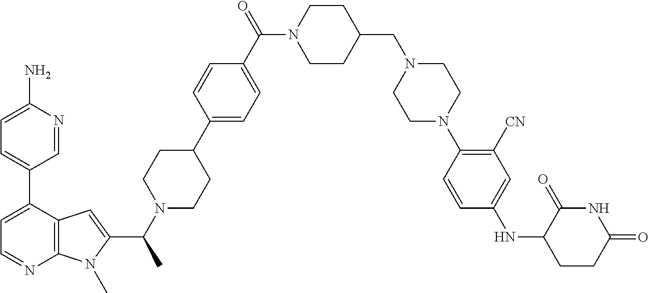 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5C#N)CC4)CC3)cc2)CC1] |
| 2i4 | 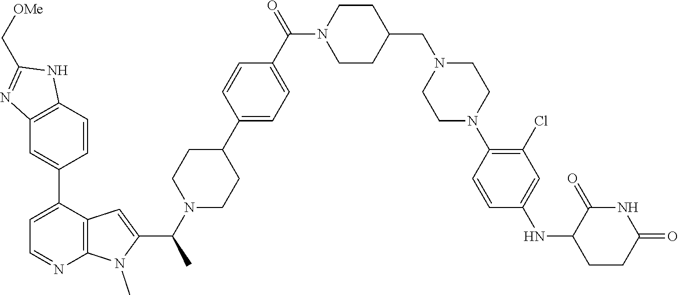 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8Cl)CC7)CC6)cc5)CC3)n4C)ccc2[nH]1] |
| 2i5 | 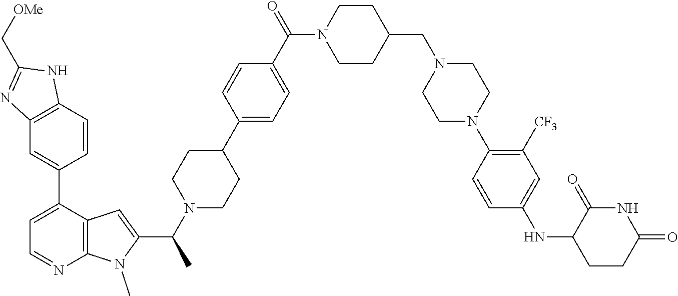 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8C(F)(F)F)CC7)CC6)cc5)CC3)n4C)ccc2[nH]1] |
| 2i6 | 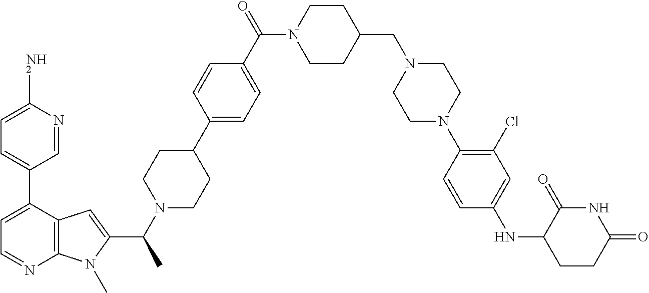 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5Cl)CC4)CC3)cc2)CC1] |
| 2i7 | 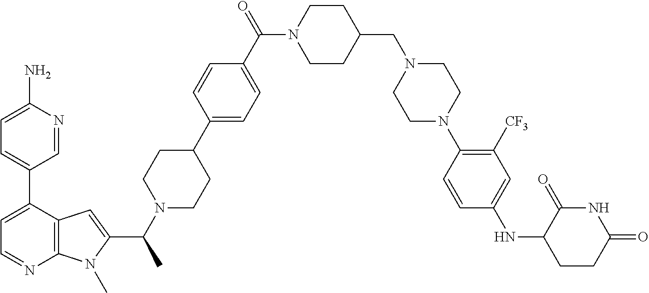 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)cc5C(F)(F)F)CC4)CC3)cc2)CC1] |
| 2j1 | 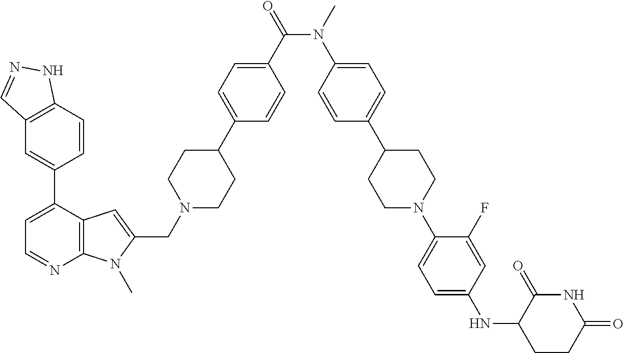 [SMILES: CN(C(=O)c1ccc(C2CCN(Cc3cc4c(-c5ccc6[nH]ncc6c5)ccnc4n3C)CC2)cc1)c1ccc(C2CCN(c3ccc(NC4CCC(=O)NC4=O)cc3F)CC2)cc1] |
| 2j2 | 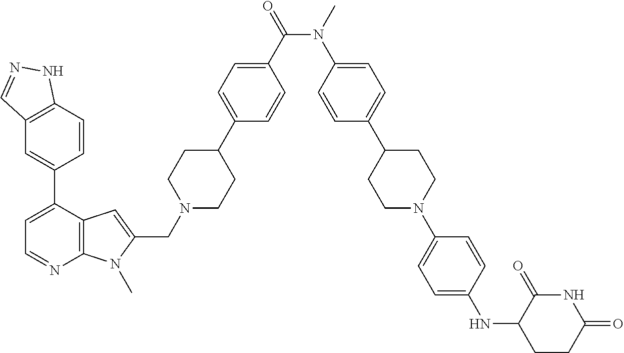 [SMILES: CN(C(=O)c1ccc(C2CCN(Cc3cc4c(-c5ccc6[nH]ncc6c5)ccnc4n3C)CC2)cc1)c1ccc(C2CCN(c3ccc(NC4CCC(=O)NC4=O)cc3)CC2)cc1] |
| 2k1 | 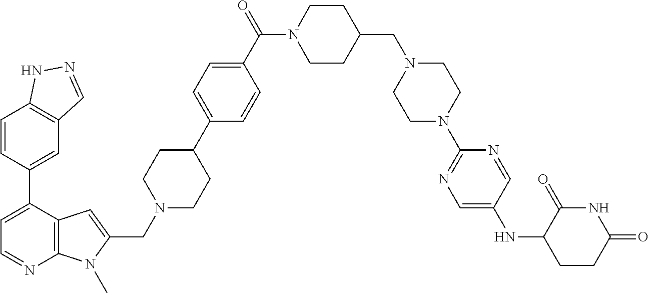 [SMILES: Cn1c(CN2CCC(c3ccc(C(=O)N4CCC(CN5CCN(c6ncc(NC7CCC(=O)NC7=O)cn6)CC5)CC4)cc3)CC2)cc2c(-c3ccc4[nH]ncc4c3)ccnc21] |
| 2k2 | 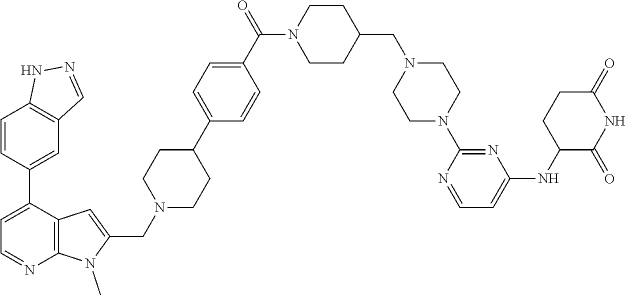 [SMILES: Cn1c(CN2CCC(c3ccc(C(=O)N4CCC(CN5CCN(c6nccc(NC7CCC(=O)NC7=O)n6)CC5)CC4)cc3)CC2)cc2c(-c3ccc4[nH]ncc4c3)ccnc21] |
| 2k3 | 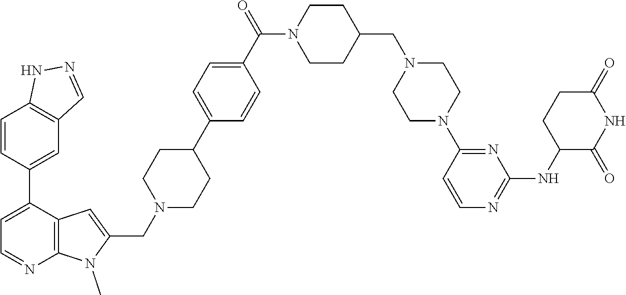 [SMILES: Cn1c(CN2CCC(c3ccc(C(=O)N4CCC(CN5CCN(c6ccnc(NC7CCC(=O)NC7=O)n6)CC5)CC4)cc3)CC2)cc2c(-c3ccc4[nH]ncc4c3)ccnc21] |
| 2l1 | 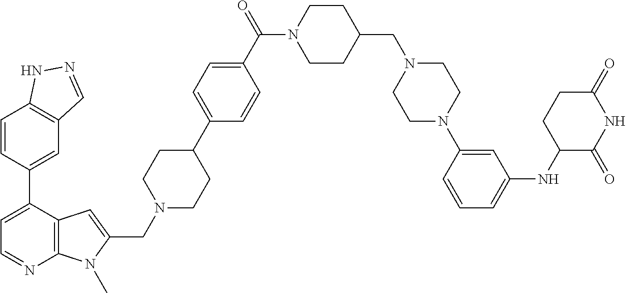 [SMILES: Cn1c(CN2CCC(c3ccc(C(=O)N4CCC(CN5CCN(c6cccc(NC7CCC(=O)NC7=O)c6)CC5)CC4)cc3)CC2)cc2c(-c3ccc4[nH]ncc4c3)ccnc21] |
| 2m1 | 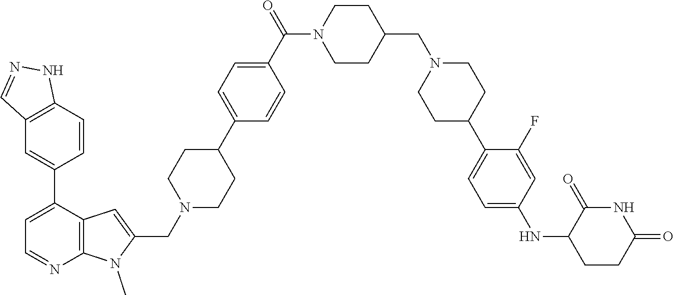 [SMILES: Cn1c(CN2CCC(c3ccc(C(=O)N4CCC(CN5CCC(c6ccc(NC7CCC(=O)NC7=O)cc6F)CC5)CC4)cc3)CC2)cc2c(-c3ccc4[nH]ncc4c3)ccnc21] |
| 2m2 | 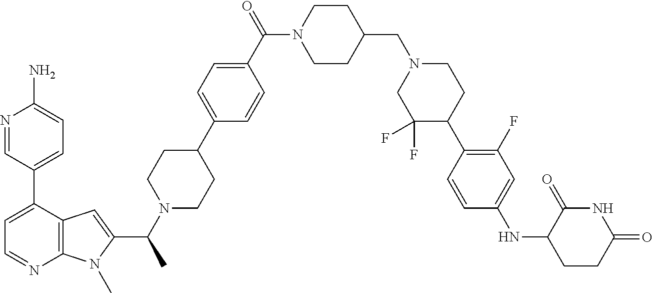 [SMILES: C[C@@H](c1cc2c(-c3ccc(N)nc3)ccnc2n1C)N1CCC(c2ccc(C(=O)N3CCC(CN4CCC(c5ccc(NC6CCC(=O)NC6=O)cc5F)C(F)(F)C4)CC3)cc2)CC1] |
| 2m3 | 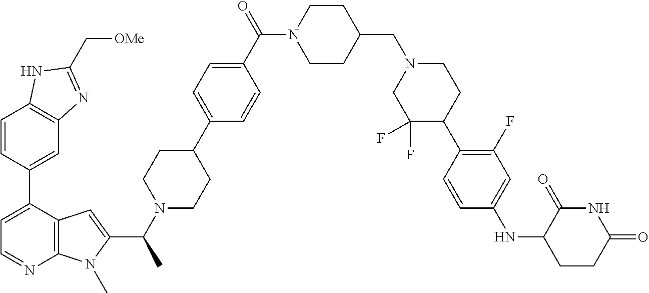 [SMILES: COCc1nc2cc(-c3ccnc4c3cc([C@H](C)N3CCC(c5ccc(C(=O)N6CCC(CN7CCC(c8ccc(NC9CCC(=O)NC9=O)cc8F)C(F)(F)C7)CC6)cc5)CC3)n4C)ccc2[nH]1] |
| 2n1 | 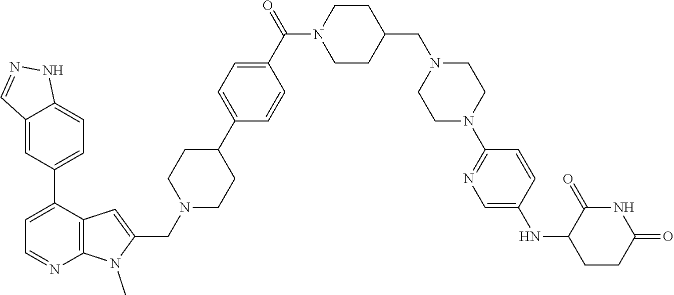 [SMILES: Cn1c(CN2CCC(c3ccc(C(=O)N4CCC(CN5CCN(c6ccc(NC7CCC(=O)NC7=O)cn6)CC5)CC4)cc3)CC2)cc2c(-c3ccc4[nH]ncc4c3)ccnc21] |
| 2n10 | 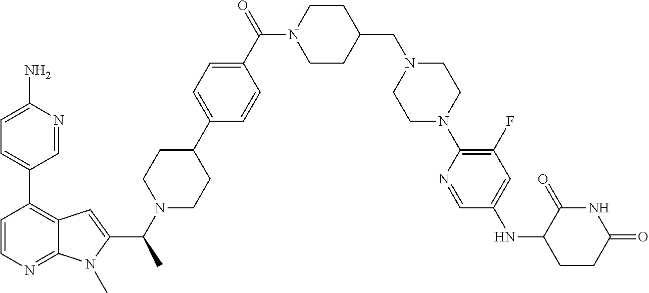 [SMILES: C[C@@H](c1cc2c(-c3ccc(N)nc3)ccnc2n1C)N1CCC(c2ccc(C(=O)N3CCC(CN4CCN(c5ncc(NC6CCC(=O)NC6=O)cc5F)CC4)CC3)cc2)CC1] |
| 2n11 | 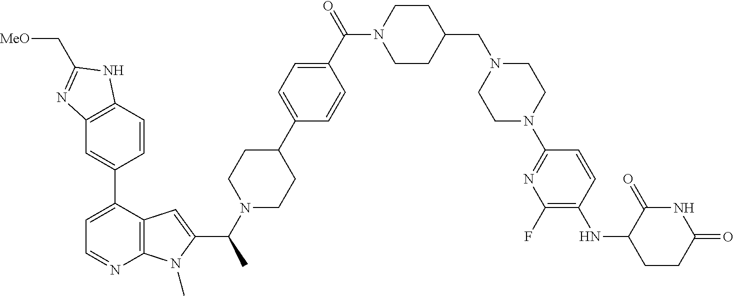 [SMILES: COCc1nc2cc(-c3ccnc4c3cc([C@H](C)N3CCC(c5ccc(C(=O)N6CCC(CN7CCN(c8ccc(NC9CCC(=O)NC9=O)c(F)n8)CC7)CC6)cc5)CC3)n4C)ccc2[nH]1] |
| 2n12 | 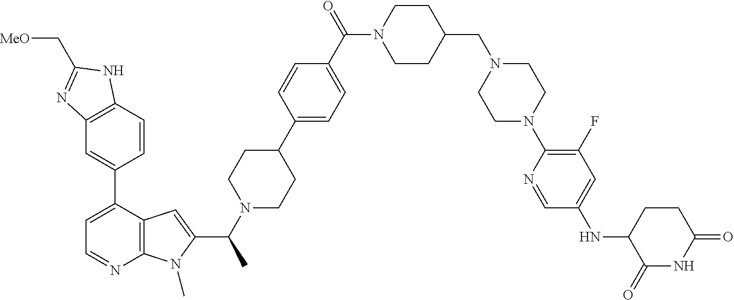 [SMILES: COCc1nc2cc(-c3ccnc4c3cc([C@H](C)N3CCC(c5ccc(C(=O)N6CCC(CN7CCN(c8ncc(NC9CCC(=O)NC9=O)cc8F)CC7)CC6)cc5)CC3)n4C)ccc2[nH]1] |
| 2n13 | 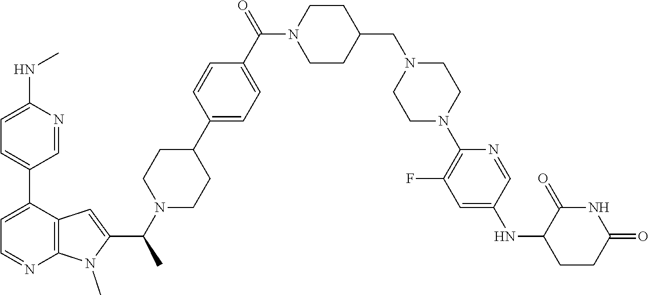 [SMILES: CNc1ccc(-c2ccnc3c2cc([C@H](C)N2CCC(c4ccc(C(=O)N5CCC(CN6CCN(c7ncc(NC8CCC(=O)NC8=O)cc7F)CC6)CC5)cc4)CC2)n3C)cn1] |
| 2n14 | 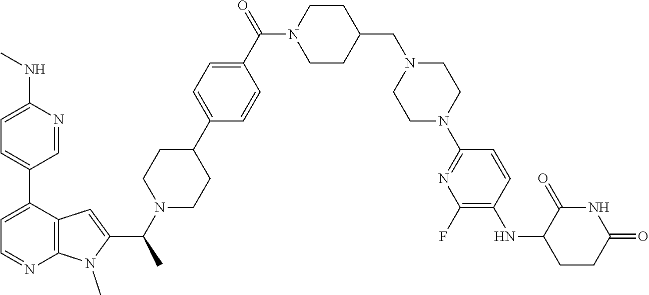 [SMILES: CNc1ccc(-c2ccnc3c2cc([C@H](C)N2CCC(c4ccc(C(=O)N5CCC(CN6CCN(c7ccc(NC8CCC(=O)NC8=O)c(F)n7)CC6)CC5)cc4)CC2)n3C)cn1] |
| 2n2 | 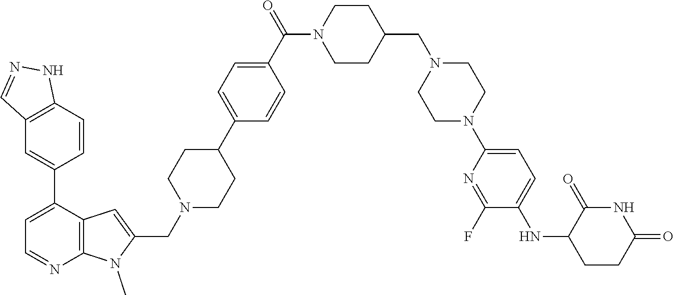 [SMILES: Cn1c(CN2CCC(c3ccc(C(=O)N4CCC(CN5CCN(c6ccc(NC7CCC(=O)NC7=O)c(F)n6)CC5)CC4)cc3)CC2)cc2c(-c3ccc4[nH]ncc4c3)ccnc21] |
| 2n3 | 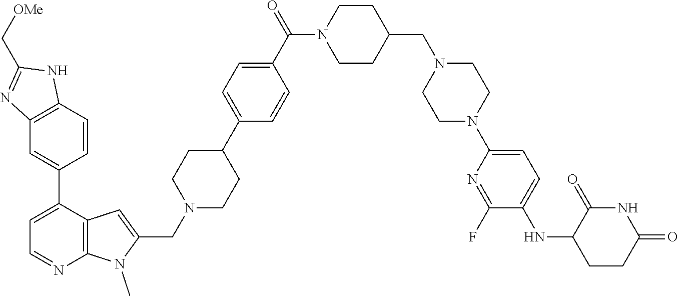 [SMILES: COCc1nc2cc(-c3ccnc4c3cc(CN3CCC(c5ccc(C(=O)N6CCC(CN7CCN(c8ccc(NC9CCC(=O)NC9=O)c(F)n8)CC7)CC6)cc5)CC3)n4C)ccc2[nH]1] |
| 2n4 | 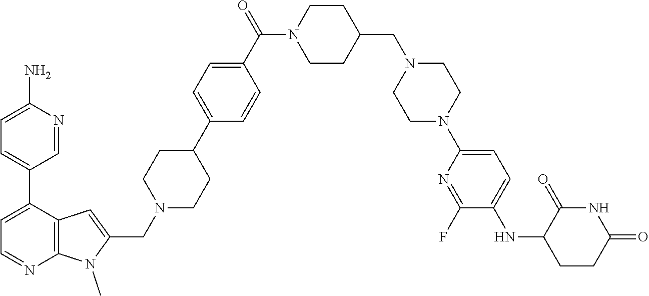 [SMILES: Cn1c(CN2CCC(c3ccc(C(=O)N4CCC(CN5CCN(c6ccc(NC7CCC(=O)NC7=O)c(F)n6)CC5)CC4)cc3)CC2)cc2c(-c3ccc(N)nc3)ccnc21] |
| 2n5 | 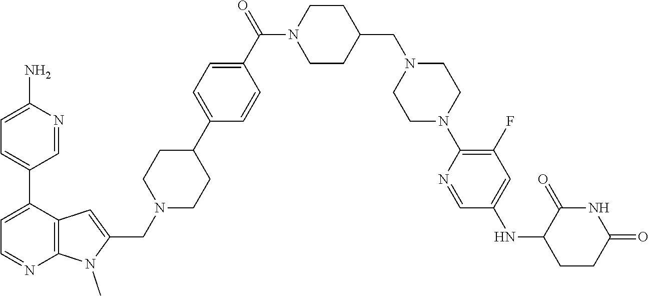 [SMILES: Cn1c(CN2CCC(c3ccc(C(=O)N4CCC(CN5CCN(c6ncc(NC7CCC(=O)NC7=O)cc6F)CC5)CC4)cc3)CC2)cc2c(-c3ccc(N)nc3)ccnc21] |
| 2n6 | 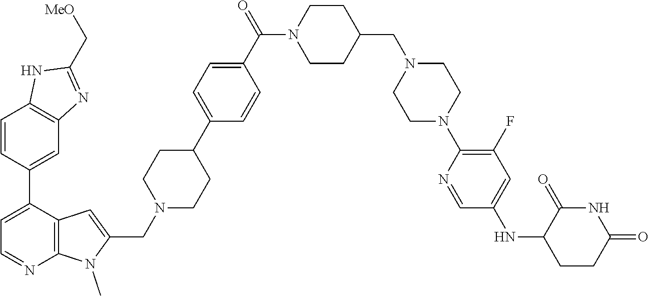 [SMILES: COCc1nc2cc(-c3ccnc4c3cc(CN3CCC(c5ccc(C(=O)N6CCC(CN7CCN(c8ncc(NC9CCC(=O)NC9=O)cc8F)CC7)CC6)cc5)CC3)n4C)ccc2[nH]1] |
| 2n7 | 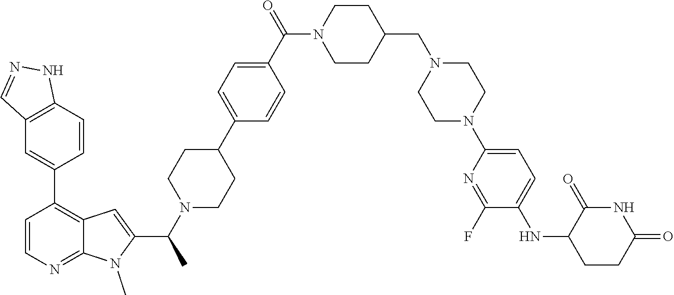 [SMILES: C[C@@H](c1cc2c(-c3ccc4[nH]ncc4c3)ccnc2n1C)N1CCC(c2ccc(C(=O)N3CCC(CN4CCN(c5ccc(NC6CCC(=O)NC6=O)c(F)n5)CC4)CC3)cc2)CC1] |
| 2n8 | 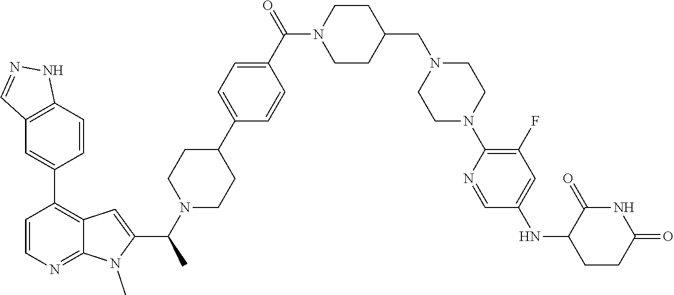 [SMILES: C[C@@H](c1cc2c(-c3ccc4[nH]ncc4c3)ccnc2n1C)N1CCC(c2ccc(C(=O)N3CCC(CN4CCN(c5ncc(NC6CCC(=O)NC6=O)cc5F)CC4)CC3)cc2)CC1] |
| 2n9 | 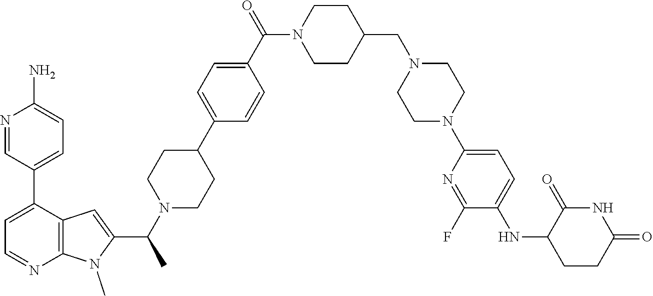 [SMILES: C[C@@H](c1cc2c(-c3ccc(N)nc3)ccnc2n1C)N1CCC(c2ccc(C(=O)N3CCC(CN4CCN(c5ccc(NC6CCC(=O)NC6=O)c(F)n5)CC4)CC3)cc2)CC1] |
| 2o1 | 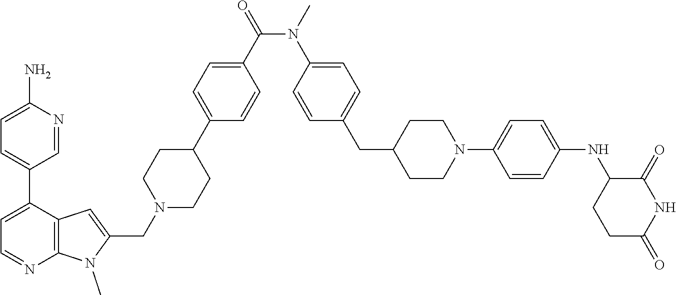 [SMILES: CN(C(=O)c1ccc(C2CCN(Cc3cc4c(-c5ccc(N)nc5)ccnc4n3C)CC2)cc1)c1ccc(CC2CCN(c3ccc(NC4CCC(=O)NC4=O)cc3)CC2)cc1] |
| 2o2 | 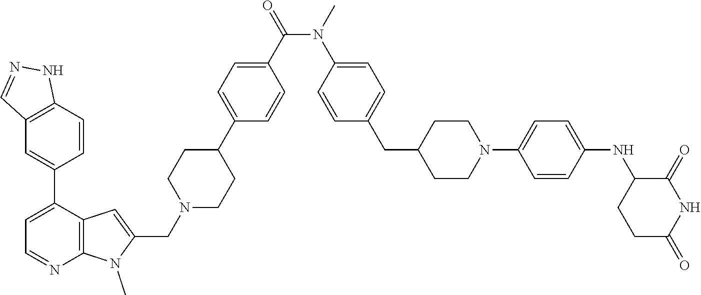 [SMILES: CN(C(=O)c1ccc(C2CCN(Cc3cc4c(-c5ccc6[nH]ncc6c5)ccnc4n3C)CC2)cc1)c1ccc(CC2CCN(c3ccc(NC4CCC(=O)NC4=O)cc3)CC2)cc1] |
| 2p1 | 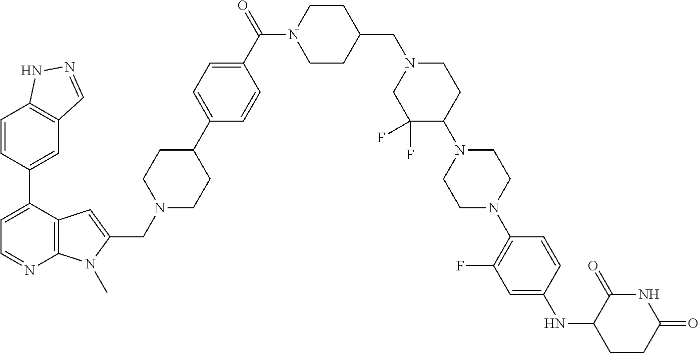 [SMILES: Cn1c(CN2CCC(c3ccc(C(=O)N4CCC(CN5CCC(N6CCN(c7ccc(NC8CCC(=O)NC8=O)cc7F)CC6)C(F)(F)C5)CC4)cc3)CC2)cc2c(-c3ccc4[nH]ncc4c3)ccnc21] |
| 2p2 | 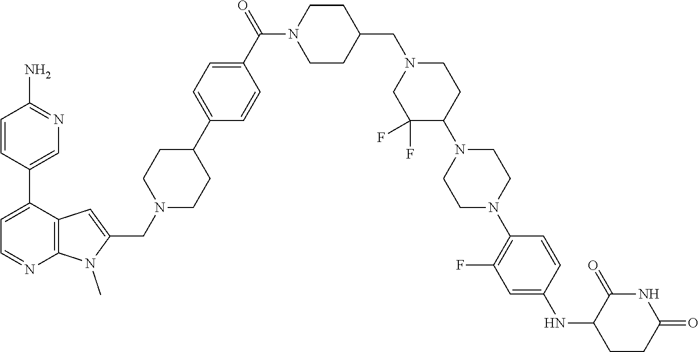 [SMILES: Cn1c(CN2CCC(c3ccc(C(=O)N4CCC(CN5CCC(N6CCN(c7ccc(NC8CCC(=O)NC8=O)cc7F)CC6)C(F)(F)C5)CC4)cc3)CC2)cc2c(-c3ccc(N)nc3)ccnc21] |
| 2p3 | 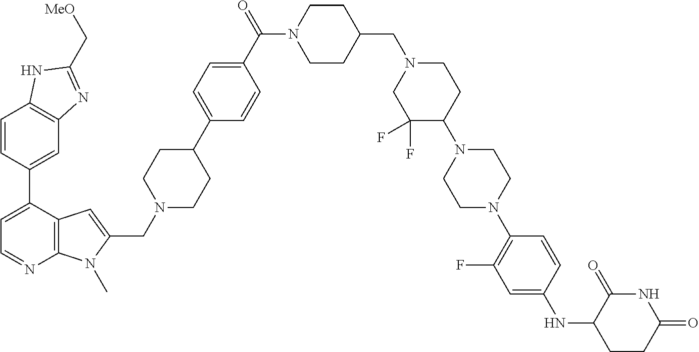 [SMILES: COCc1nc2cc(-c3ccnc4c3cc(CN3CCC(c5ccc(C(=O)N6CCC(CN7CCC(N8CCN(c9ccc(NC%10CCC(=O)NC%10=O)cc9F)CC8)C(F)(F)C7)CC6)cc5)CC3)n4C)ccc2[nH]1] |
| 2q1 | 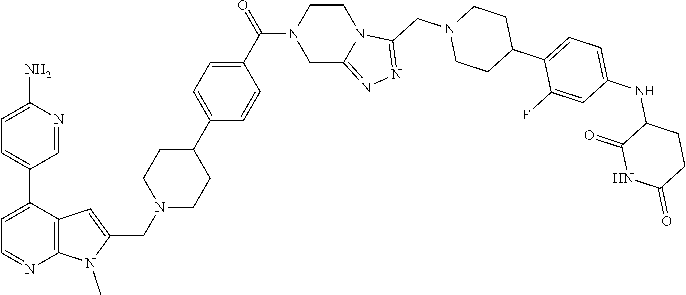 [SMILES: Cn1c(CN2CCC(c3ccc(C(=O)N4CCn5c(CN6CCC(c7ccc(NC8CCC(=O)NC8=O)cc7F)CC6)nnc5C4)cc3)CC2)cc2c(-c3ccc(N)nc3)ccnc21] |
| 2q2 | 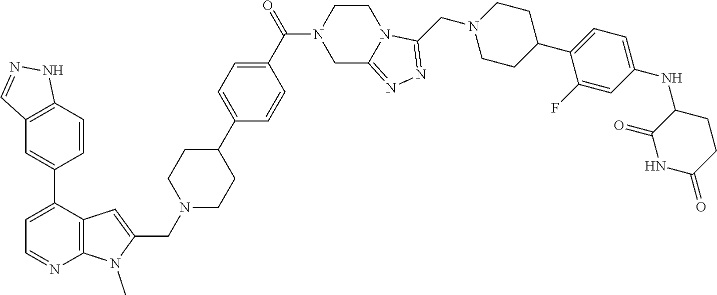 [SMILES: Cn1c(CN2CCC(c3ccc(C(=O)N4CCn5c(CN6CCC(c7ccc(NC8CCC(=O)NC8=O)cc7F)CC6)nnc5C4)cc3)CC2)cc2c(-c3ccc4[nH]ncc4c3)ccnc21] |
| 2q3 | 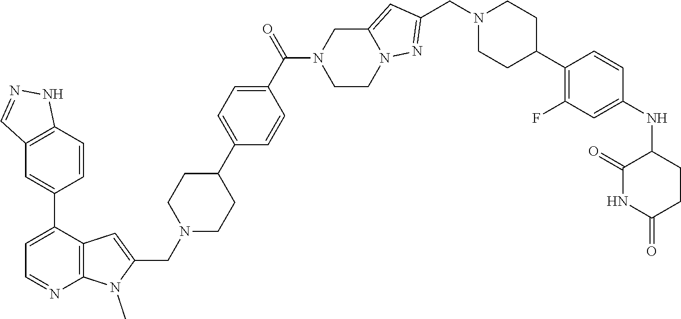 [SMILES: Cn1c(CN2CCC(c3ccc(C(=O)N4CCn5nc(CN6CCC(c7ccc(NC8CCC(=O)NC8=O)cc7F)CC6)cc5C4)cc3)CC2)cc2c(-c3ccc4[nH]ncc4c3)ccnc21] |
| 2q4 | 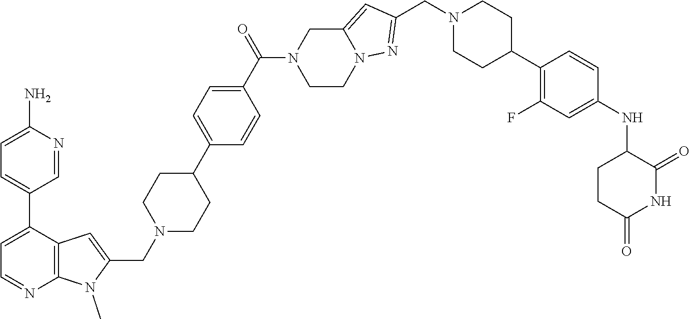 [SMILES: Cn1c(CN2CCC(c3ccc(C(=O)N4CCn5nc(CN6CCC(c7ccc(NC8CCC(=O)NC8=O)cc7F)CC6)cc5C4)cc3)CC2)cc2c(-c3ccc(N)nc3)ccnc21] |
| 2q5 | 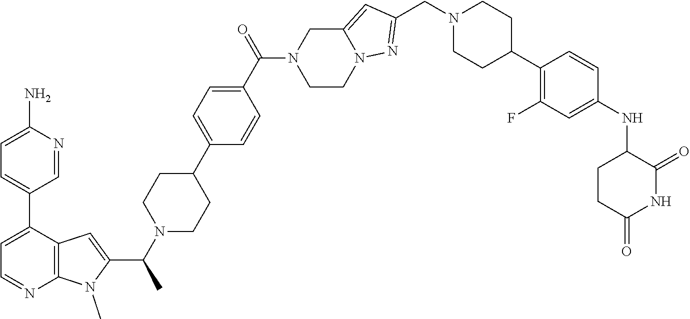 [SMILES: C[C@@H](c1cc2c(-c3ccc(N)nc3)ccnc2n1C)N1CCC(c2ccc(C(=O)N3CCn4nc(CN5CCC(c6ccc(NC7CCC(=O)NC7=O)cc6F)CC5)cc4C3)cc2)CC1] |
| 2q6 | 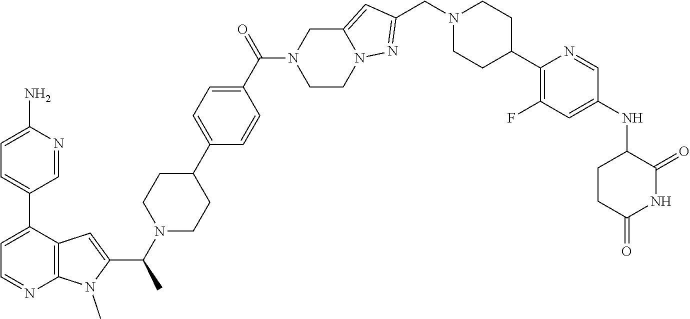 [SMILES: C[C@@H](c1cc2c(-c3ccc(N)nc3)ccnc2n1C)N1CCC(c2ccc(C(=O)N3CCn4nc(CN5CCC(c6ncc(NC7CCC(=O)NC7=O)cc6F)CC5)cc4C3)cc2)CC1] |
| 2q7 | 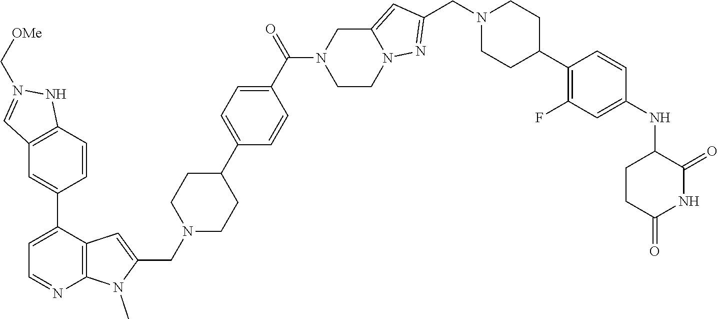 |
| 2q8 | 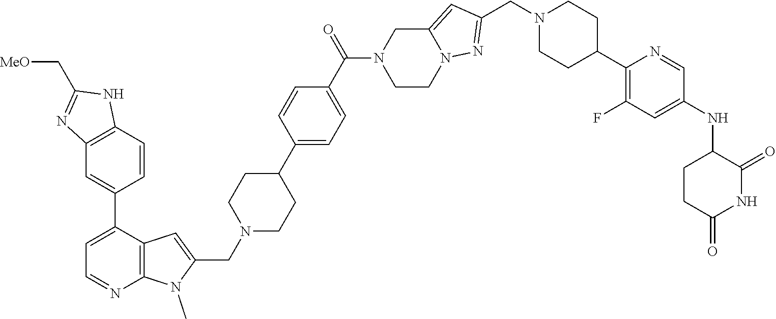 [SMILES: COCc1nc2cc(-c3ccnc4c3cc(CN3CCC(c5ccc(C(=O)N6CCn7nc(CN8CCC(c9ncc(NC%10CCC(=O)NC%10=O)cc9F)CC8)cc7C6)cc5)CC3)n4C)ccc2[nH]1] |
| 2q9 | 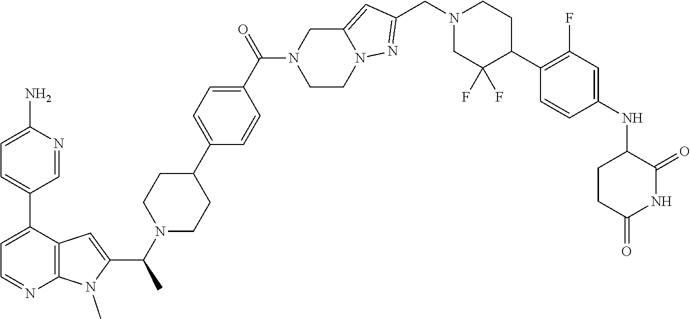 [SMILES: C[C@@H](c1cc2c(-c3ccc(N)nc3)ccnc2n1C)N1CCC(c2ccc(C(=O)N3CCn4nc(CN5CCC(c6ccc(NC7CCC(=O)NC7=O)cc6F)C(F)(F)C5)cc4C3)cc2)CC1] |
| 2q10 | 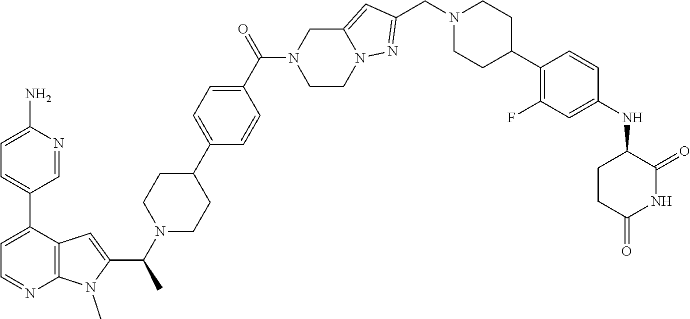 [SMILES: C[C@@H](c1cc2c(-c3ccc(N)nc3)ccnc2n1C)N1CCC(c2ccc(C(=O)N3CCn4nc(CN5CCC(c6ccc(N[C@@H]7CCC(=O)NC7=O)cc6F)CC5)cc4C3)cc2)CC1] |
| 2q11 | 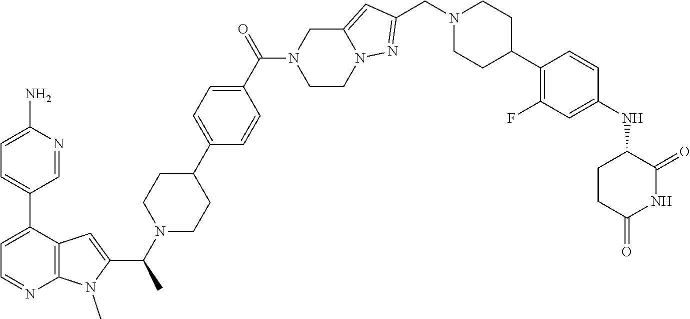 [SMILES: C[C@@H](c1cc2c(-c3ccc(N)nc3)ccnc2n1C)N1CCC(c2ccc(C(=O)N3CCn4nc(CN5CCC(c6ccc(N[C@H]7CCC(=O)NC7=O)cc6F)CC5)cc4C3)cc2)CC1] |
| 2q12 | 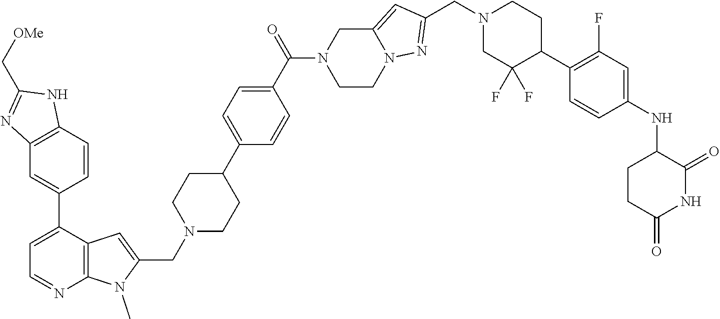 [SMILES: COCc1nc2cc(-c3ccnc4c3cc(CN3CCC(c5ccc(C(=O)N6CCn7nc(CN8CCC(c9ccc(NC%10CCC(=O)NC%10=O)cc9F)C(F)(F)C8)cc7C6)cc5)CC3)n4C)ccc2[nH]1] |
| 2q14 | 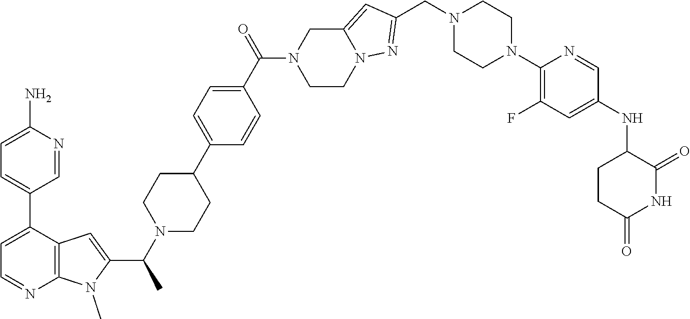 [SMILES: C[C@@H](c1cc2c(-c3ccc(N)nc3)ccnc2n1C)N1CCC(c2ccc(C(=O)N3CCn4nc(CN5CCN(c6ncc(NC7CCC(=O)NC7=O)cc6F)CC5)cc4C3)cc2)CC1] |
| 2q15 | 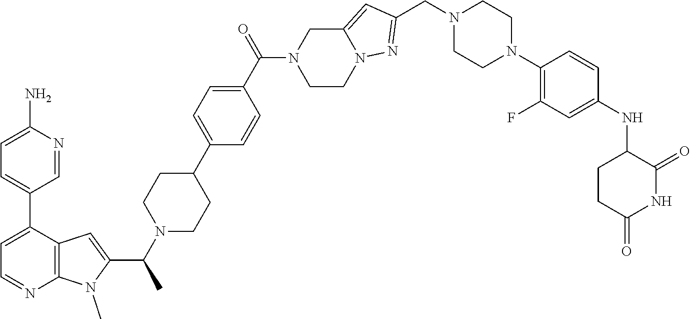 [SMILES: C[C@@H](c1cc2c(-c3ccc(N)nc3)ccnc2n1C)N1CCC(c2ccc(C(=O)N3CCn4nc(CN5CCN(c6ccc(NC7CCC(=O)NC7=O)cc6F)CC5)cc4C3)cc2)CC1] |
| 2r1 | 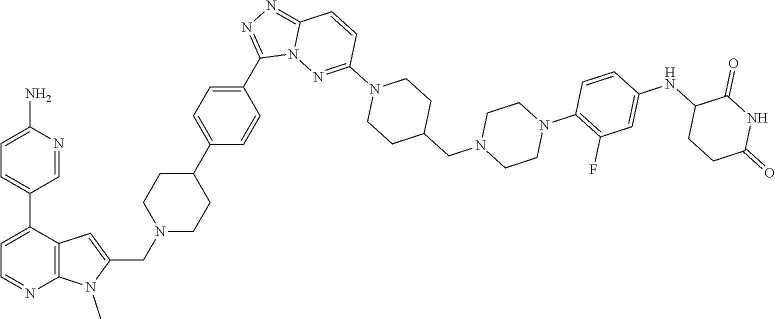 [SMILES: Cn1c(CN2CCC(c3ccc(-c4nnc5ccc(N6CCC(CN7CCN(c8ccc(NC9CCC(=O)NC9=O)cc8F)CC7)CC6)nn45)cc3)CC2)cc2c(-c3ccc(N)nc3)ccnc21] |
| 2s1 | 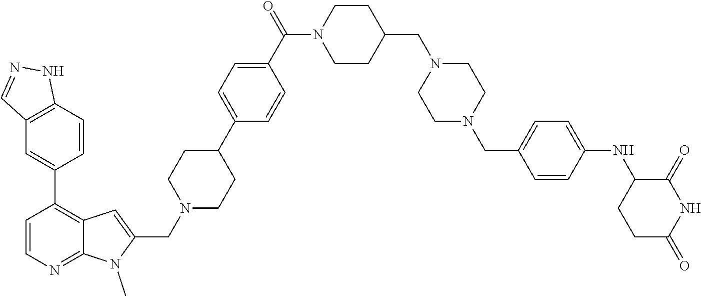 [SMILES: Cn1c(CN2CCC(c3ccc(C(=O)N4CCC(CN5CCN(Cc6ccc(NC7CCC(=O)NC7=O)cc6)CC5)CC4)cc3)CC2)cc2c(-c3ccc4[nH]ncc4c3)ccnc21] |
| 2s2 | 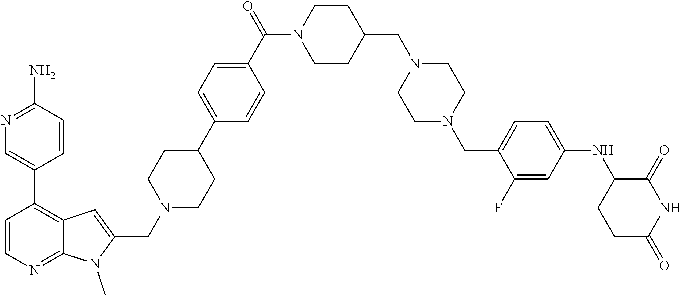 [SMILES: Cn1c(CN2CCC(c3ccc(C(=O)N4CCC(CN5CCN(Cc6ccc(NC7CCC(=O)NC7=O)cc6F)CC5)CC4)cc3)CC2)cc2c(-c3ccc(N)nc3)ccnc21] |
| 2t1 | 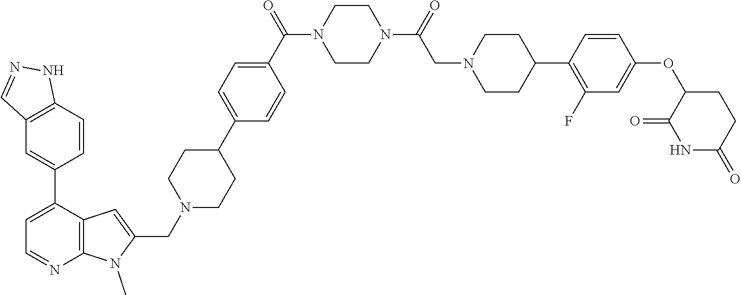 [SMILES: Cn1c(CN2CCC(c3ccc(C(=O)N4CCN(C(=O)CN5CCC(c6ccc(OC7CCC(=O)NC7=O)cc6F)CC5)CC4)cc3)CC2)cc2c(-c3ccc4[nH]ncc4c3)ccnc21] |
| 2t2 | 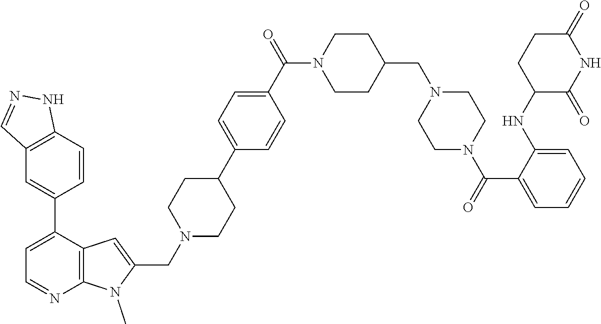 [SMILES: Cn1c(CN2CCC(c3ccc(C(=O)N4CCC(CN5CCN(C(=O)c6ccccc6NC6CCC(=O)NC6=O)CC5)CC4)cc3)CC2)cc2c(-c3ccc4[nH]ncc4c3)ccnc21] |
| 2t3 | 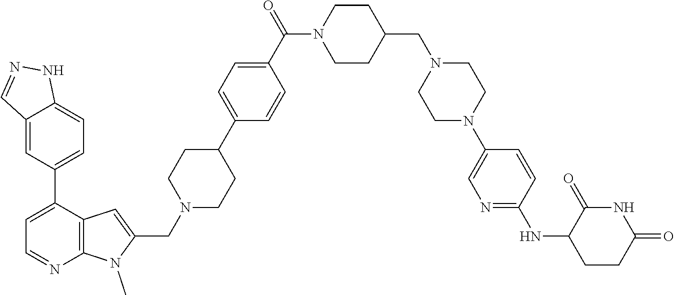 [SMILES: Cn1c(CN2CCC(c3ccc(C(=O)N4CCC(CN5CCN(c6ccc(NC7CCC(=O)NC7=O)nc6)CC5)CC4)cc3)CC2)cc2c(-c3ccc4[nH]ncc4c3)ccnc21] |
| 2t4 | 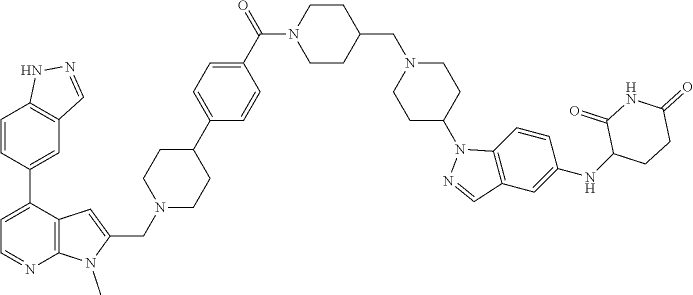 [SMILES: Cn1c(CN2CCC(c3ccc(C(=O)N4CCC(CN5CCC(n6ncc7cc(NC8CCC(=O)NC8=O)ccc76)CC5)CC4)cc3)CC2)cc2c(-c3ccc4[nH]ncc4c3)ccnc21] |
| 2t5 | 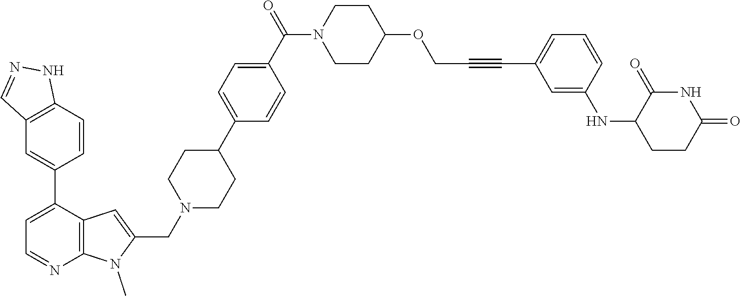 [SMILES: Cn1c(CN2CCC(c3ccc(C(=O)N4CCC(OCC#Cc5cccc(NC6CCC(=O)NC6=O)c5)CC4)cc3)CC2)cc2c(-c3ccc4[nH]ncc4c3)ccnc21] |
| | and |
| 3t5 | 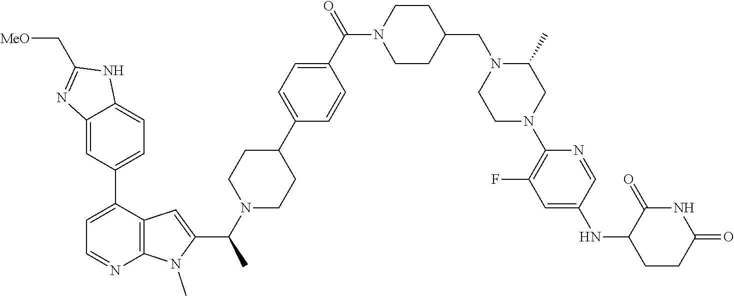 [SMILES: COCc1nc2cc(-c3ccnc4c3cc([C@H](C)N3CCC(c5ccc(C(=O)N6CCC(CN7CCN(c8ncc(NC9CCC(=O)NC9=O)cc8F)C[C@H]7C)CC6)cc5)CC3)n4C)ccc2[nH]1] |

or a pharmaceutically acceptable salt or stereoisomer thereof.

21. A method of treating a disease, disorder or condition in a patient in need thereof, the method comprising administering to the patient a therapeutically effective amount of a compound of claim 1, or a pharmaceutically acceptable salt or stereoisomer thereof, which disease, disorder or condition is responsive of modulation of STAT-6.

22. The method of claim 21, wherein the disease is an autoimmune disease.

23. A method of treating or ameliorating a disease in a patient in need thereof, the method comprising administering to the patient a therapeutically effective amount of a compound of claim 1, or a pharmaceutically acceptable salt or stereoisomer thereof, wherein said disease is characterized by Th2-mediated inflammation such as atopic dermatitis, asthma, chronic rhinosinusitis with nasal polyposis, urticaria, rhinitis, eosinophilic esophagitis, food allergy, diffuse cutaneous systemic sclerosis, alopecia areata and/or COPD (chronic obstructive pulmonary disease) and different cancers such as lymphomas, triple negative breast and solid fibrous cancers either as a stand-alone treatment or in combination with other anticancer drugs such as check point inhibitors.

24.-27. (canceled)

28. A pharmaceutical composition comprising a compound according to claim 1, together with a pharmaceutically acceptable vehicle or excipient or pharmaceutically acceptable carrier(s).

29. A method of treating a disease or condition mediated by interleukin 4 (IL-4) or interleukin 3 (IL-3) in a patient in need thereof, the method comprising administering to the patient a therapeutically effective amount of a compound of claim 1 or a pharmaceutically acceptable salt or stereoisomer thereof.
